(19) United States
(12) Patent Application Publication
(10) Pub. No.: US 2025/0188069 A1
(43) Pub. Date: Jun. 12, 2025

(54) COMPOUNDS AND METHODS FOR DEGRADING CASEIN KINASE 1 ALPHA

(71) Applicant: PIN THERAPEUTICS, INC., Seongnam-si, Gyeonggi-do (KR)

(72) Inventors: Hyunsun JO, Seongnam-si (KR); Nicholas Kenneth TERRETT, Lambertville, NJ (US); Daniel Emil LEVY, San Mateo, CA (US); Michael Paul DENINNO, Gales Ferry, CT (US); John KING-UNDERWOOD, Pendock (GB); Jun-Jae CHUNG, South San Francisco, CA (US); Sarina MOHANTY, Pasadena, CA (US); Jisu PARK, Seongnam-si (KR); Purum KIM, Seongnam-si (KR); yuna PARK, Seongnam-si (KR); Firoz Ali JAIPURI, Fremont, CA (US)

(73) Assignee: PIN THERAPEUTICS, INC., Seongnam-si, Gyeonggi-do (KR)

(21) Appl. No.: 18/876,352

(22) PCT Filed: Jun. 27, 2023
(86) PCT No.: PCT/IB2023/056639

§ 371 Date: Dec. 18, 2024

Related U.S. Application Data
(60) Provisional application No. 63/355,840, filed on Jun. 27, 2022.
(60) Provisional application No. 63/447,528, filed on Feb. 22, 2023.
(60) Provisional application No. 63/471,723, filed on Jun. 7, 2023.

(51) Int. Cl. C07D 413/14; A61K 31/454; A61K 31/4545; A61K 31/496; A61K 31/5377; C07D 401/04; C07D 401/14; C07D 405/14; C07D 493/04

(52) U.S. Cl. CPC C07D 413/14; A61K 31/454; A61K 31/4545; A61K 31/496; A61K 31/5377; C07D 401/04; C07D 401/14; C07D 405/14; C07D 493/04

(57) ABSTRACT

Provided are novel compounds represented by Formula (I) which degrade CK1α (casein kinase 1 alpha), and methods of degrading CK1α, or preventing or treating blood cancer by administering the compounds to a subject in need thereof.

### BACKGROUND OF THE INVENTION

Casein kinase 1 alpha (CK1α) is a serine/threonine protein kinase involved in various cellular pathways and functions, including Wnt signaling, NF-κB signaling, p53 pathway, autophagy, and cell cycle. Inhibition of CK1a or reduction of its expression level has been demonstrated to induce cell death in several types of cancer, such as acute myeloid leukemia (AML) and diffuse large B cell lymphoma (DLBCL).

For instance, it has been reported that CK1α inhibition causes reduced Rps6 phosphorylation and activation of p53, resulting in selective elimination of leukemia cells, revealing CK1α as a therapeutic target for the treatment of AML (Jaras et al., J. Exp. Med. 2014 Vol. 211 No. 4 605-612). In addition, it was reported that in AML and multiple myeloma (MM), inhibition of CK1α, acting via activation of p53 pathway, showed promising preclinical activities (Janovska et al., Int. J. Mol. Sci. 2020, 21, 9026). In particular, it was previously found that CK1α controls signaling pathways involved in proliferation, survival and stress in MM (Manni et al., Journal of Hematology & Oncology (2017) 10:157). Furthermore, it has been reported that CK1α is overexpressed in AML patients and acts as a negative element in the prognosis of AML patients, and thus CK1α inhibits p53 downstream of MDM2-mediated autophagy and apoptosis, and the targeting of CK1α and autophagy may offer a therapeutic opportunity to treat AML (Xu et al., ONCOLOGY REPORTS 44:1895-1904, 2020). It has been also previously found that CK1α governs antigen-receptor-induced NF-κB activation and human lymphoma cell survival.

Therefore, CK1α has been known in the art as a target for preventing or treating blood cancer such as leukemia, lymphoma, and myeloma. Therefore, there has been a demand for developing CK1α-degraders or inhibitors which may be used for preventing or treating blood cancer such as leukemia, lymphoma, and myeloma.

### SUMMARY OF THE INVENTION

The present disclosure provides novel compounds which are represented by Formula (I) as follows, or a pharmaceutically acceptable salt, enantiomer, stereoisomer or prodrug thereof.

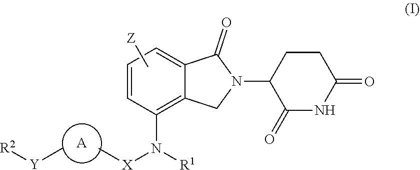

Such a compound of Formula (I) or a pharmaceutically acceptable salt, enantiomer, stereoisomer, or prodrug thereof is useful for reducing CK1α protein levels and for treating or preventing blood cancer.

In Formula (I), in some embodiment, R¹ is C₁-C₈alkyl, C₃-C₈ alkenyl, C₃-C₈ alkynyl, C₃-C₈ cycloalkyl or C₅-C₁₀ bicycloalkyl each optionally substituted with one or more halogen, OH, O—C₁-C₃ alkyl, NH₂, C₁-C₃ haloalkyl, optionally substituted C₆-C₁₀ aryl, C₁-C₃ alkyl, optionally substituted C₃-C₈ cycloalkyl, optionally substituted C₅-C₁₀ bicycloalkyl or optionally substituted C₃-C₆ heterocycloalkyl.

In some embodiment, X is a bond or —(CH₂)ₙ— optionally substituted with one or more halogen, OH, O—C₁-C₃ alkyl, C₁-C₃ haloalkyl, C₃-C₆ cycloalkyl or C₁-C₆ alkyl, wherein the C₁-C₆ alkyl group may be taken together with the atom to which it is attached to form a C₃-C₆ spiro alkyl ring, and wherein the —(CH₂)ₙ— group may contain 0-1 double bond or triple bond.

In some embodiment, n is an integer from 1 to 6.

In some embodiment, A is a bond, C₄-C₈ cycloalkyl, C₅-C₁₀ bicycloalkyl, C₅-C₁₀ heterobicycloalkyl or C₃-C₈ heterocycloalkyl each optionally substituted with one of more halogen, OH, O—C₁-C₃ alkyl, CN, C₁-C₃ haloalkyl, C₁-C₃ alkyl or C₃-C₅ cycloalkyl.

In some embodiment, Y is a bond or a —(CH₂)ₘ—.

In some embodiment, m is an integer of 1 or 2.

In some embodiment, Z is H or halogen.

In some embodiment, R² is —NR¹²R⁵, —NHC(O)R⁶, —NHC(O)NR⁸(R⁹), —C(O)NHR⁵, five or six-membered heteroaryl, or C₃-C₅ heterocycloalkyl or C₅-C₁₀ heterobicycloalkyl ring optionally substituted with one of more halogen, OH, O—C₁-C₃ alkyl, CN, C₁-C₃ haloalkyl, C₁-C₃ alkyl or C₃-C₅ cycloalkyl.

In some embodiment, R⁵ is H, C₁-C₅ alkyl, C₃-C₇ cycloalkyl, five or six-membered heteroaryl, C₅-C₁₀ bicycloalkyl, (C₃-C₆ cycloalkyl)-C₁-C₃ alkyl, (C₃-C₆ heterocycloalkyl)-C₁-C₃ alkyl, (C₆-C₁₀ aryl)-C₁-C₃ alkyl, (C₁-C₅-heteroaryl)-C₁-C₃ alkyl or C₃-C₇ heterocycloalkyl each optionally substituted with one or more halogen, CN, OH, O—(C₁-C₃ haloalkyl), O—(C₁-C₃ alkyl), C₁-C₃ haloalkyl, CH₃SO₂—, optionally substituted C₃-C₆ cycloalkyl, optionally substituted C₃-C₇ heterocycloalkyl or C(O)NR⁸(R⁹).

In some embodiment, R⁶ is C₁-C₆ alkyl, C₃-C₆ cycloalkyl or C₃-C₆ heterocycloalkyl each optionally substituted with one or more halogen, C₁-C₃ haloalkyl, optionally substituted C₃-C₆ cycloalkyl, or NR⁸(R⁹).

In some embodiment, R⁸ and R⁹ are each independently H, substituted C₁-C₃ alkyl or taken together with the nitrogen to which they are attached to form a 4-6 membered heterocyclic ring optionally substituted with one or more halogen or C₁-C₃ haloalkyl.

In some embodiment, R¹² is H, C₁-C₅ alkyl, or taken together with R⁵ and the nitrogen to which it is attached to form (i) C₃-C₅ cycloalkyl or heterocycloalkyl ring or (ii) C₅-C₁₀ bicycloalkyl or heterobicycloalkyl ring, each optionally substituted with one or more halogen, C₁-C₃ haloalkyl, OH, O—(C₁-C₃ alkyl), O—(C₁-C₃ haloalkyl) or CN.

In some embodiment, each stereocenter in the compound of Formula (I) is independently the R-enantiomer, the S-enantiomer or a mixture of R- and S-enantiomers.

In some embodiment, each double bond in the compound of Formula (I) is independently cis or trans.

In some embodiment, the compound is represented by Formula (I-a),

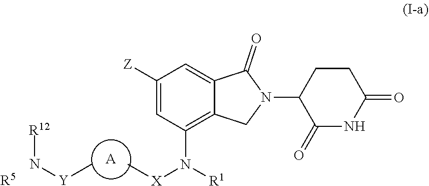

or a pharmaceutically acceptable salt, enantiomer, stereoisomer or prodrug thereof.

In Formula (I-a), in some embodiment, X is a bond or —(CH₂)ₙ—.

In some embodiment, R¹ is C₁-C₅ alkyl or C₃-C₅ cycloalkyl each optionally substituted with one or more halogen, OH, NH₂, C₁-C₃ alkyl, C₁-C₃ haloalkyl, O—(C₁-C₃ alkyl), optionally substituted C₆ aryl, optionally substituted C₃-C₅ cycloalkyl, optionally substituted C₃-C₆ heterocycloalkyl or optionally substituted C₆-C₅ bicycloalkyl.

In some embodiment, A is C₄-C₆ cycloalkyl, C₄-C₆ heterocycloalkyl or C₅-C₇ bicycloalkyl each optionally substituted with one or more halogen, OH, C₁-C₃ alkyl or C₁-C₃ haloalkyl.

In some embodiment, Y is a bond or —CH₂—.

In some embodiment, R⁵ is H, C₁-C₅ alkyl, C₃-C₆ cycloalkyl, 6-membered heteroaryl, C₅ bicycloalkyl, (C₃-C₄ cycloalkyl)-C₁-C₂ alkyl, (C₃-C₄ heterocycloalkyl)-C₁-C₂ alkyl, (C₆ aryl)-C₁-C₂ alkyl, (C₅ heteroaryl)-C₁-C₂ alkyl or C₃-C₄ heterocycloalkyl each optionally substituted with one or more halogen, CN, OH, O—(C₁-C₃ haloalkyl), O—(C₁-C₃ alkyl), C₁-C₃ haloalkyl, CH₃SO₂— or C(O)NR⁸(R⁹).

In some embodiment, R¹² is H, C₁-C₅ alkyl, or taken together with R⁵ and the nitrogen to which it is attached to form a C₃-C₅ heterocycloalkyl ring optionally substituted with one or more halogen or C₁-C₃ haloalkyl.

In some embodiment, the compound is represented by Formula (I-b),

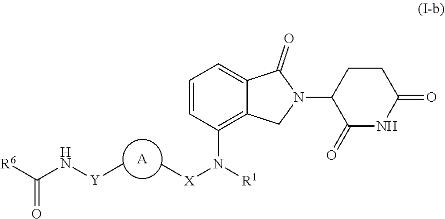

or a pharmaceutically acceptable salt, enantiomer, stereoisomer or prodrug thereof.

In Formula (I-b), in some embodiment, X is a bond or —(CH₂)ₙ—.

In some embodiment, R¹ is C₃-C₆ alkyl optionally substituted with one or more halogen, OH, O—C₁-C₃ alkyl, NH₂, C₁-C₃ haloalkyl.

In some embodiment, A is bond or C₄-C₆ cycloalkyl optionally substituted with one or more halogen, OH, O—C₁-C₃ alkyl, NH₂, C₁-C₃ haloalkyl.

In some embodiment, Y is a bond.

In some embodiment, R⁶ is C₁-C₆ alkyl, optionally substituted with NR⁸(R⁹).

In some embodiment, the compound is represented by Formula (I-c),

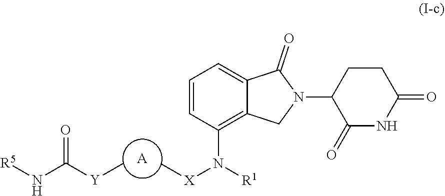

or a pharmaceutically acceptable salt, enantiomer, stereoisomer or prodrug thereof.

In Formula (I-c), in some embodiment, X is a bond or —(CH₂)ₙ—.

In some embodiment, R¹ is C₁-C₆ alkyl optionally substituted with optionally substituted C₃-C₅ cycloalkyl or optionally substituted C₃-C₆ heterocycloalkyl.

In some embodiment, A is bond, C₄-C₆ cycloalkyl, C₄-C₆ heterocycloalkyl or C₅-C₇ bicycloalkyl each optionally substituted with one or more halogen, OH, O—C₁-C₃ alkyl, NH₂, C₁-C₃ haloalkyl.

In some embodiment, Y is a bond.

In some embodiment, R⁵ is H or C₁-C₃ alkyl.

In some embodiment, the compound is represented by Formula (I-d),

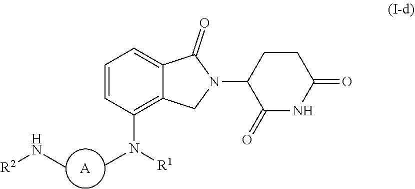

or a pharmaceutically acceptable salt, enantiomer, stereoisomer or prodrug thereof.

In Formula (I-d), in some embodiment, R¹ is C₁-C₃ alkyl optionally substituted with optionally substituted C₃-C₆ cycloalkyl.

In some embodiment, A is optionally substituted C₄-C₆ cycloalkyl.

In some embodiment, R² is C₁-C₄ alkyl or (C₃-C₄ cycloalkyl)-C₁-C₂ alkyl, each optionally substituted with one or more halogen, CN, OH, O—(C₁-C₃ haloalkyl), O—(C₁-C₃ alkyl), C₁-C₃ haloalkyl or CH₃SO₂—.

In Formula (I), In some embodiment, each X and Y is a bond; A is C₄-C₆ cycloalkyl; R¹ is C₁-C₃ alkyl optionally substituted with optionally substituted C₃-C₆ cycloalkyl; and R² is 5-membered heteroaryl.

In some embodiment, the compound is selected from the following Table A.

TABLE A

| Compounds | | |
|---|---|---|
| No. | Structure | Name |
| 2 | 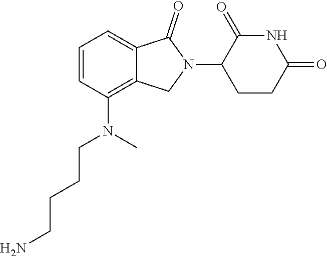 [SMILES: CN(CCCCN)c1cccc2c1CN(C1CCC(=O)NC1=O)C2=O] | 3-(4-((4-aminobutyl)(methyl)amino)-1- oxoisoindolin-2-yl)piperidine-2,6- dione |
| 3 | 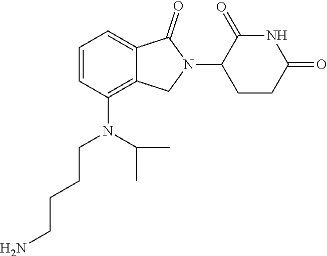 [SMILES: CC(C)N(CCCCN)c1cccc2c1CN(C1CCC(=O)NC1=O)C2=O] | 3-(4-((4- aminobutyl)(isopropyl)amino)-1- oxoisoindolin-2-yl)piperidine-2,6- dione |
| 4 | 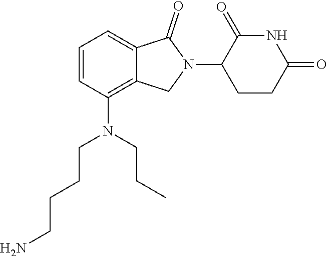 [SMILES: CCCN(CCCCN)c1cccc2c1CN(C1CCC(=O)NC1=O)C2=O] | 3-(4-((4-aminobutyl)(propyl)amino)-1- oxoisoindolin-2-yl)piperidine-2,6- dione |
| 46 | 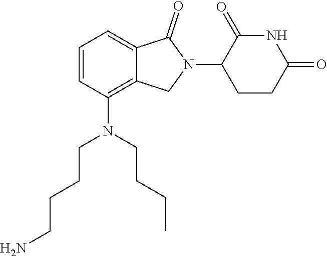 [SMILES: CCCCN(CCCCN)c1cccc2c1CN(C1CCC(=O)NC1=O)C2=O] | 3-(4-((4-aminobutyl)(butyl)amino)-1- oxoisoindolin-2-yl)piperidine-2,6- dione |
| 47 | 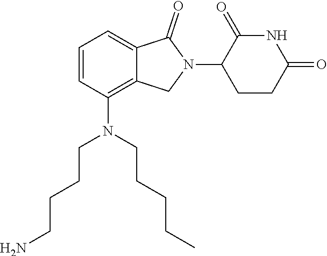 [SMILES: CCCCCN(CCCCN)c1cccc2c1CN(C1CCC(=O)NC1=O)C2=O] | 3-(4-((4-aminobutyl)(pentyl)amino)-1- oxoisoindolin-2-yl)piperidine-2,6- dione |
| 48 | 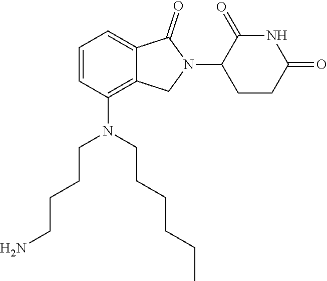 [SMILES: CCCCCCN(CCCCN)c1cccc2c1CN(C1CCC(=O)NC1=O)C2=O] | 3-(4-((4-aminobutyl)(hexyl)amino)-1- oxoisoindolin-2-yl)piperidine-2,6- dione |
| 49 | 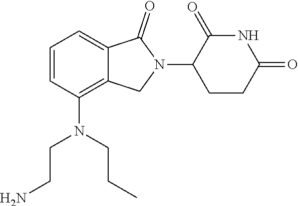 [SMILES: CCCN(CCN)c1cccc2c1CN(C1CCC(=O)NC1=O)C2=O] | 3-(4-((2-aminoethyl)(propyl)amino)-1- oxoisoindolin-2-yl)piperidine-2,6- dione |
| 50 | 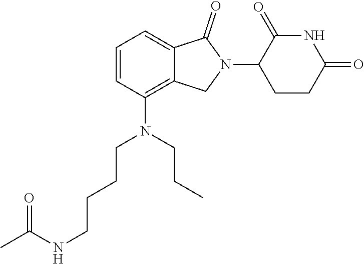 [SMILES: CCCN(CCCCNC(C)=O)c1cccc2c1CN(C1CCC(=O)NC1=O)C2=O] | N-(4-((2-(2,6-dioxopiperidin-3-yl)-1- oxoisoindolin-4- yl)(propyl)amino)butyl)acetamide |
| 51 | 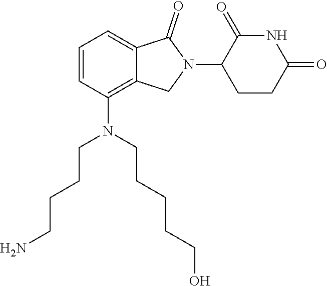 [SMILES: NCCCCN(CCCCCO)c1cccc2c1CN(C1CCC(=O)NC1=O)C2=O] | 3-(4-((4-aminobutyl)(5- hydroxypentyl)amino)-1- oxoisoindolin-2-yl)piperidine-2,6- dione |
| 52 | 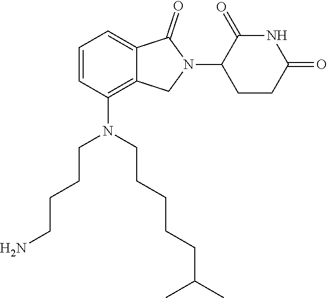 [SMILES: CC(C)CCCCCN(CCCCN)c1cccc2c1CN(C1CCC(=O)NC1=O)C2=O] | 3-(4-((4-aminobutyl)(6- methylheptyl)amino)-1-oxoisoindolin- 2-yl)piperidine-2,6-dione |
| 53 | 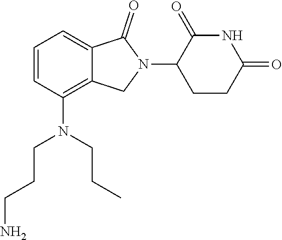 [SMILES: CCCN(CCCN)c1cccc2c1CN(C1CCC(=O)NC1=O)C2=O] | 3-(4-((3-aminopropyl)(propyl)amino)- 1-oxoisoindolin-2-yl)piperidine-2,6- dione |
| 55 | 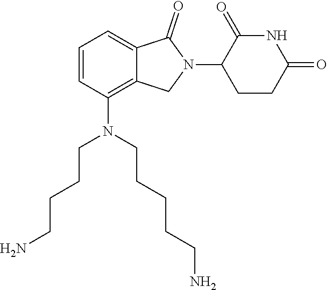 [SMILES: NCCCCCN(CCCCN)c1cccc2c1CN(C1CCC(=O)NC1=O)C2=O] | 3-(4-((4-aminobutyl)(5- aminopentyl)amino)-1-oxoisoindolin- 2-yl)piperidine-2,6-dione |
| 56 | 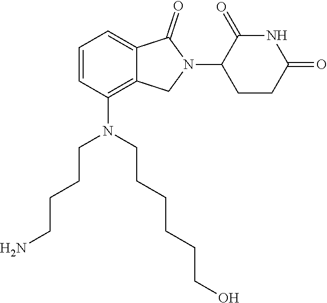 [SMILES: NCCCCN(CCCCCCO)c1cccc2c1CN(C1CCC(=O)NC1=O)C2=O] | 3-(4-((4-aminobutyl)(6- hydroxyhexyl)amino)-1-oxoisoindolin- 2-yl)piperidine-2,6-dione |
| 57 | 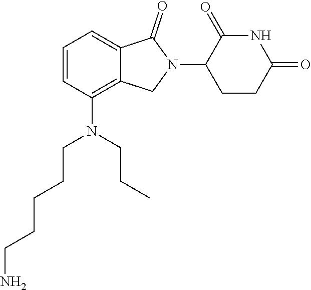 [SMILES: CCCN(CCCCCN)c1cccc2c1CN(C1CCC(=O)NC1=O)C2=O] | 3-(4-((5-aminopentyl)(propyl)amino)- 1-oxoisoindolin-2-yl)piperidine-2,6- dione |
| 60 | 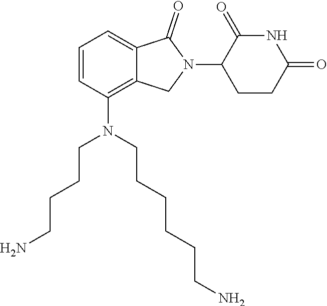 [SMILES: NCCCCCCN(CCCCN)c1cccc2c1CN(C1CCC(=O)NC1=O)C2=O] | 3-(4-((4-aminobutyl)(6- aminohexyl)amino)-1-oxoisoindolin-2- yl)piperidine-2,6-dione |
| 63 | 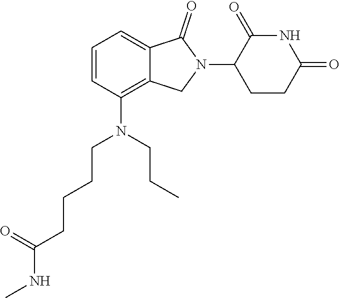 [SMILES: CCCN(CCCCC(=O)NC)c1cccc2c1CN(C1CCC(=O)NC1=O)C2=O] | 5-((2-(2,6-dioxopiperidin-3-yl)-1- oxoisoindolin-4-yl)(propyl)amino)-N- methylpentanamide |
| 66 | 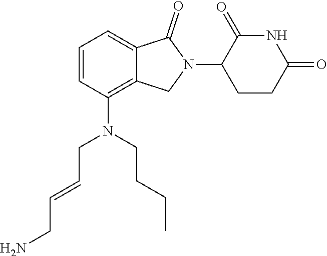 [SMILES: CCCCN(C/C=C/CN)c1cccc2c1CN(C1CCC(=O)NC1=O)C2=O] | (E)-3-(4-((4-aminobut-2-en-1- yl)(butyl)amino)-1-oxoisoindolin-2- yl)piperidine-2,6-dione |
| 67 | 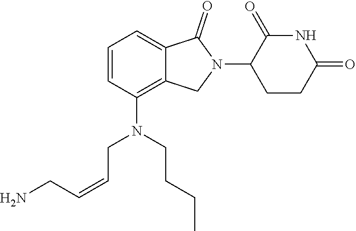 [SMILES: CCCCN(C/C=C\CN)c1cccc2c1CN(C1CCC(=O)NC1=O)C2=O] | (Z)-3-(4-((4-aminobut-2-en-1- yl)(butyl)amino)-1-oxoisoindolin-2- yl)piperidine-2,6-dione |
| 68 | 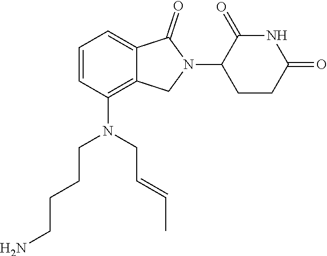 [SMILES: C/C=C/CN(CCCCN)c1cccc2c1CN(C1CCC(=O)NC1=O)C2=O] | (E)-3-(4-((4-aminobutyl)(but-2-en-1- yl)amino)-1-oxoisoindolin-2- yl)piperidine-2,6-dione |
| 69 | 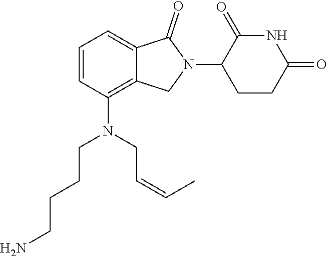 [SMILES: C/C=C\CN(CCCCN)c1cccc2c1CN(C1CCC(=O)NC1=O)C2=O] | (Z)-3-(4-((4-aminobutyl)(but-2-en-1- yl)amino)-1-oxoisoindolin-2- yl)piperidine-2,6-dione |
| 80 | 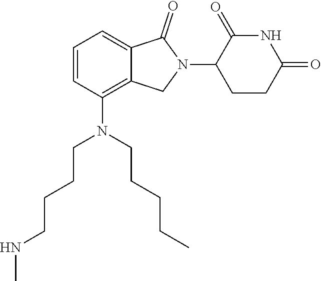 [SMILES: CCCCCN(CCCCNC)c1cccc2c1CN(C1CCC(=O)NC1=O)C2=O] | 3-(4-((4- (methylamino)butyl)(pentyl)amino)-1- oxoisoindolin-2-yl)piperidine-2,6- dione |
| 81 | 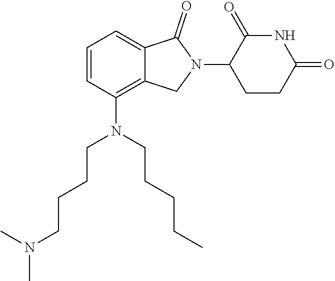 [SMILES: CCCCCN(CCCCN(C)C)c1cccc2c1CN(C1CCC(=O)NC1=O)C2=O] | 3-(4-((4- (dimethylamino)butyl)(pentyl)amino)- 1-oxoisoindolin-2-yl)piperidine-2,6- dione |
| 82 | 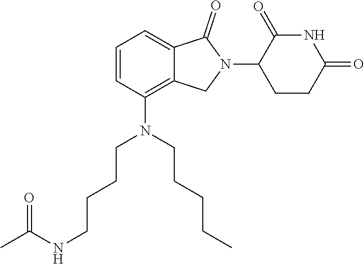 [SMILES: CCCCCN(CCCCNC(C)=O)c1cccc2c1CN(C1CCC(=O)NC1=O)C2=O] | N-(4-((2-(2,6-dioxopiperidin-3-yl)-1- oxoisoindolin-4- yl)(pentyl)amino)butyl)acetamide |
| 87 | 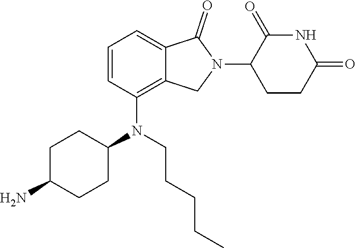 [SMILES: CCCCCN(c1cccc2c1CN(C1CCC(=O)NC1=O)C2=O)[C@H]1CC[C@@H](N)CC1] | 3-(4-(((1s,4s)-4- aminocyclohexyl)(pentyl)amino)-1- oxoisoindolin-2-yl)piperidine-2,6- dione |
| 88 | 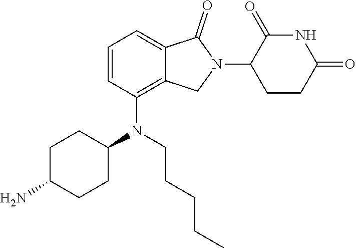 [SMILES: CCCCCN(c1cccc2c1CN(C1CCC(=O)NC1=O)C2=O)[C@H]1CC[C@H](N)CC1] | 3-(4-(((1r,4r)-4- aminocyclohexyl)(pentyl)amino)-1- oxoisoindolin-2-yl)piperidine-2,6- dione |
| 89 | 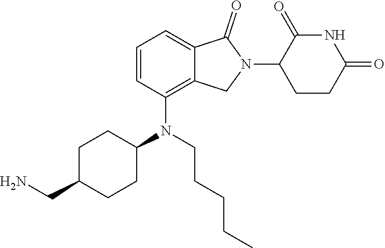 [SMILES: CCCCCN(c1cccc2c1CN(C1CCC(=O)NC1=O)C2=O)[C@H]1CC[C@@H](CN)CC1] | 3-(4-(((1s,4s)-4- (aminomethyl)cyclohexyl)(pentyl)amino)- 1-oxoisoindolin-2-yl)piperidine-2,6- dione |
| 90 | 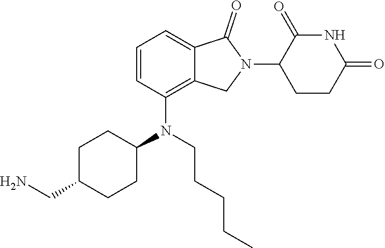 [SMILES: CCCCCN(c1cccc2c1CN(C1CCC(=O)NC1=O)C2=O)[C@H]1CC[C@H](CN)CC1] | 3-(4-(((1r,4r)-4- (aminomethyl)cyclohexyl)(pentyl)amino)- 1-oxoisoindolin-2-yl)piperidine-2,6- dione |
| 91 | 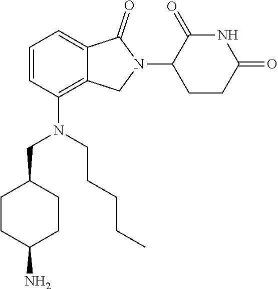 [SMILES: CCCCCN(C[C@H]1CC[C@@H](N)CC1)c1cccc2c1CN(C1CCC(=O)NC1=O)C2=O] | 3-(4-((((1s,4s)-4- aminocyclohexyl)methyl)(pentyl)amino)- 1-oxoisoindolin-2-yl)piperidine-2,6- dione |
| 92 | 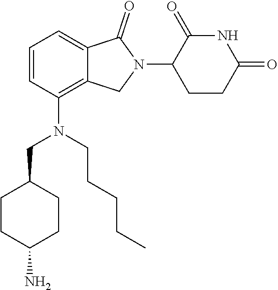 [SMILES: CCCCCN(C[C@H]1CC[C@H](N)CC1)c1cccc2c1CN(C1CCC(=O)NC1=O)C2=O] | 3-(4-((((1r,4r)-4- aminocyclohexyl)methyl)(pentyl)amino)- 1-oxoisoindolin-2-yl)piperidine-2,6- dione |
| 93 | 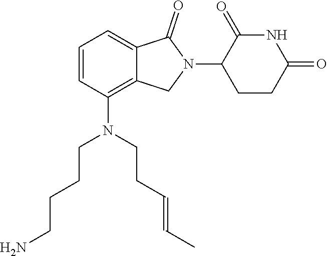 [SMILES: C/C=C/CCN(CCCCN)c1cccc2c1CN(C1CCC(=O)NC1=O)C2=O] | (E)-3-(4-((4-aminobutyl)(pent-3-en-1- yl)amino)-1-oxoisoindolin-2- yl)piperidine-2,6-dione |
| 94 | 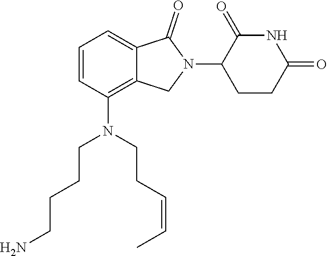 [SMILES: C/C=C\CCN(CCCCN)c1cccc2c1CN(C1CCC(=O)NC1=O)C2=O] | (Z)-3-(4-((4-aminobutyl)(pent-3-en-1- yl)amino)-1-oxoisoindolin-2- yl)piperidine-2,6-dione |
| 95 | 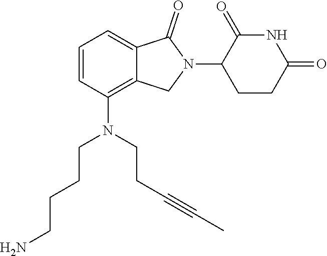 [SMILES: CC#CCCN(CCCCN)c1cccc2c1CN(C1CCC(=O)NC1=O)C2=O] | 3-(4-((4-aminobutyl)(pent-3-yn-1- yl)amino)-1-oxoisoindolin-2- yl)piperidine-2,6-dione |
| 96 | 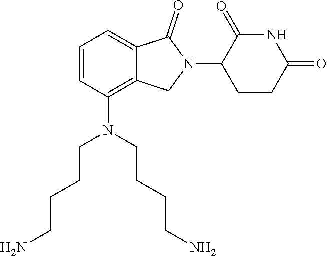 [SMILES: NCCCCN(CCCCN)c1cccc2c1CN(C1CCC(=O)NC1=O)C2=O] | 3-(4-(bis(4-aminobutyl)amino)-1- oxoisoindolin-2-yl)piperidine-2,6- dione |
| 97 | 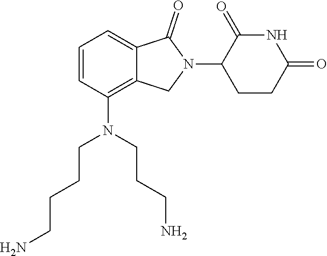 [SMILES: NCCCCN(CCCN)c1cccc2c1CN(C1CCC(=O)NC1=O)C2=O] | 3-(4-((4-aminobutyl)(3- aminopropyl)amino)-1-oxoisoindolin- 2-yl)piperidine-2,6-dione |
| 102 | 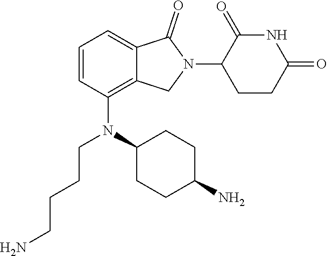 [SMILES: NCCCCN(c1cccc2c1CN(C1CCC(=O)NC1=O)C2=O)[C@H]1CC[C@@H](N)CC1] | 3-(4-((4-aminobutyl)((1s,4s)-4- aminocyclohexyl)amino)-1- oxoisoindolin-2-yl)piperidine-2,6- dione |
| 103 | 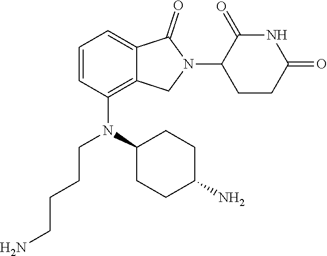 [SMILES: NCCCCN(c1cccc2c1CN(C1CCC(=O)NC1=O)C2=O)[C@H]1CC[C@H](N)CC1] | 3-(4-((4-aminobutyl)((1r,4r)-4- aminocyclohexyl)amino)-1- oxoisoindolin-2-yl)piperidine-2,6- dione |
| 104 | 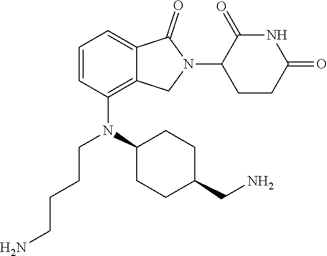 [SMILES: NCCCCN(c1cccc2c1CN(C1CCC(=O)NC1=O)C2=O)[C@H]1CC[C@@H](CN)CC1] | 3-(4-((4-aminobutyl)((1s,4s)-4- (aminomethyl)cyclohexyl)amino)-1- oxoisoindolin-2-yl)piperidine-2,6- dione |
| 105 | 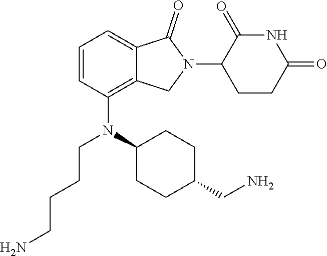 [SMILES: NCCCCN(c1cccc2c1CN(C1CCC(=O)NC1=O)C2=O)[C@H]1CC[C@H](CN)CC1] | 3-(4-((4-aminobutyl)((lr,4r)-4- (aminomethyl)cyclohexyl)amino)-1- oxoisoindolin-2-yl)piperidine-2,6- dione |
| 106 | 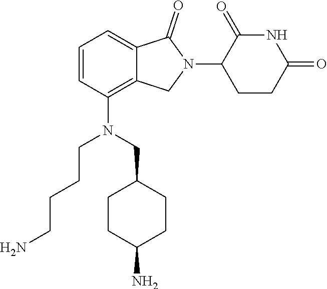 [SMILES: NCCCCN(C[C@H]1CC[C@@H](N)CC1)c1cccc2c1CN(C1CCC(=O)NC1=O)C2=O] | 3-(4-((4-aminobutyl)(((1s,4s)-4- aminocyclohexyl)methyl)amino)-1- oxoisoindolin-2-yl)piperidine-2,6- dione |
| 107 | 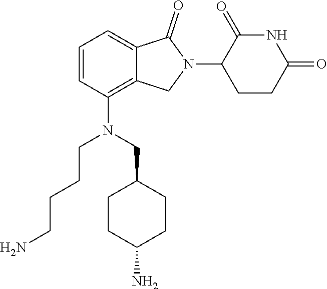 [SMILES: NCCCCN(C[C@H]1CC[C@H](N)CC1)c1cccc2c1CN(C1CCC(=O)NC1=O)C2=O] | 3-(4-((4-aminobutyl)(((1r,4r)-4- aminocyclohexyl)methyl)amino)-1- oxoisoindolin-2-yl)piperidine-2,6- dione |
| 108 | 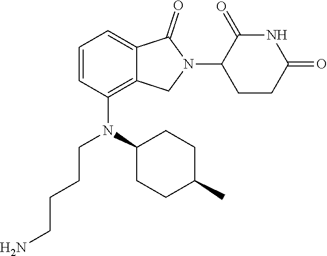 [SMILES: C[C@H]1CC[C@@H](N(CCCCN)c2cccc3c2CN(C2CCC(=O)NC2=O)C3=O)CC1] | 3-(4-((4-aminobutyl)((1s,4s)-4- methylcyclohexyl)amino)-1- oxoisoindolin-2-yl)piperidine-2,6- dione |
| 109 | 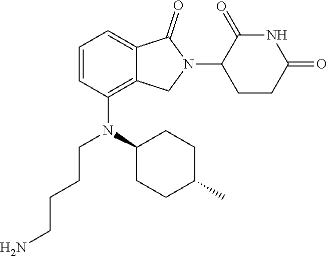 [SMILES: C[C@H]1CC[C@H](N(CCCCN)c2cccc3c2CN(C2CCC(=O)NC2=O)C3=O)CC1] | 3-(4-((4-aminobutyl)((lr,4r)-4- methylcyclohexyl)amino)-1- oxoisoindolin-2-yl)piperidine-2,6- dione |
| 115 | 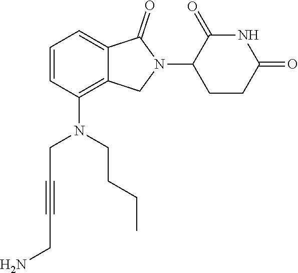 [SMILES: CCCCN(CC#CCN)c1cccc2c1CN(C1CCC(=O)NC1=O)C2=O] | 3-(4-((4-aminobut-2-yn-1- yl)(butyl)amino)-1-oxoisoindolin-2- yl)piperidine-2,6-dione |
| 116 | 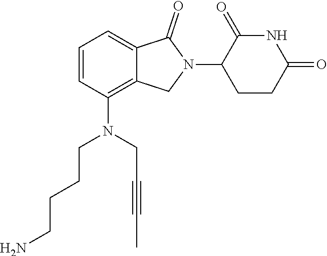 [SMILES: CC#CCN(CCCCN)c1cccc2c1CN(C1CCC(=O)NC1=O)C2=O] | 3-(4-((4-aminobutyl)(but-2-yn-1- yl)amino)-1-oxoisoindolin-2- yl)piperidine-2,6-dione |
| 123 | 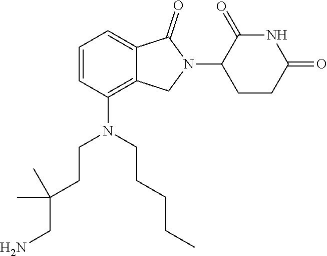 [SMILES: CCCCCN(CCC(C)(C)CN)c1cccc2c1CN(C1CCC(=O)NC1=O)C2=O] | 3-(4-((4-amino-3,3- dimethylbutyl)(pentyl)amino)-1- oxoisoindolin-2-yl)piperidine-2,6- dione |
| 124 | 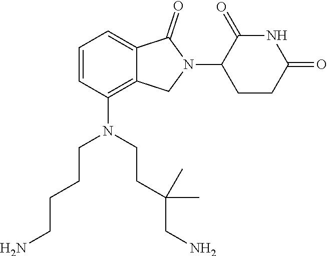 [SMILES: CC(C)(CN)CCN(CCCCN)c1cccc2c1CN(C1CCC(=O)NC1=O)C2=O] | 3-(4-((4-amino-3,3-dimethylbutyl)(4- aminobutyl)amino)-1-oxoisoindolin-2- yl)piperidine-2,6-dione |
| 125 | 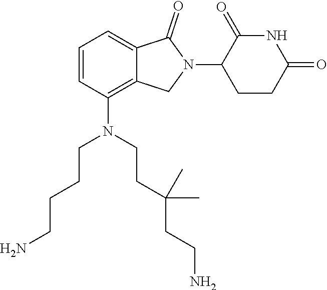 [SMILES: CC(C)(CCN)CCN(CCCCN)c1cccc2c1CN(C1CCC(=O)NC1=O)C2=O] | 3-(4-((5-amino-3,3-dimethylpentyl)(4- aminobutyl)amino)-1-oxoisoindolin-2- yl)piperidine-2,6-dione |
| 126 | 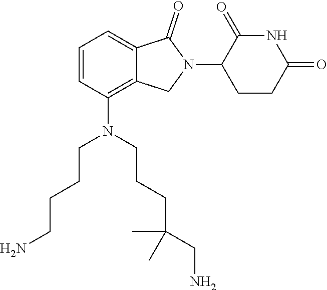 [SMILES: CC(C)(CN)CCCN(CCCCN)c1cccc2c1CN(C1CCC(=O)NC1=O)C2=O] | 3-(4-((5-amino-4,4-dimethylpentyl)(4- aminobutyl)amino)-1-oxoisoindolin-2- yl)piperidine-2,6-dione |
| 143 | 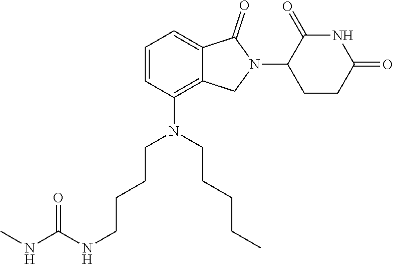 [SMILES: CCCCCN(CCCCNC(=O)NC)c1cccc2c1CN(C1CCC(=O)NC1=O)C2=O] | 1-(4-((2-(2,6-dioxopiperidin-3-yl)-1- oxoisoindolin-4- yl)(pentyl)amino)butyl)-3-methylurea |
| 148 | 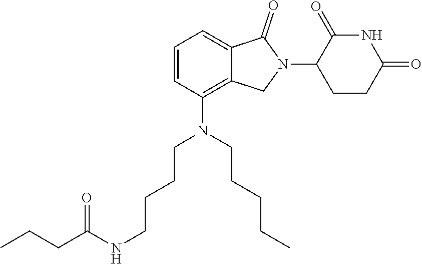 [SMILES: CCCCCN(CCCCNC(=O)CCC)c1cccc2c1CN(C1CCC(=O)NC1=O)C2=O] | N-(4-((2-(2,6-dioxopiperidin-3-yl)-1- oxoisoindolin-4- yl)(pentyl)amino)butyl)butyramide |
| 149 | 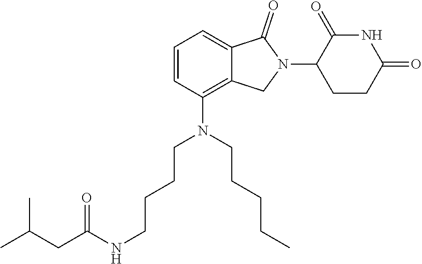 [SMILES: CCCCCN(CCCCNC(=O)CC(C)C)c1cccc2c1CN(C1CCC(=O)NC1=O)C2=O] | N-(4-((2-(2,6-dioxopiperidin-3-yl)-1- oxoisoindolin-4- yl)(pentyl)amino)butyl)-3- methylbutanamide |
| 150 | 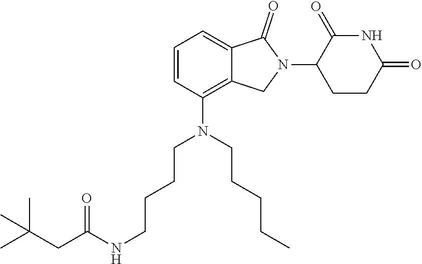 [SMILES: CCCCCN(CCCCNC(=O)CC(C)(C)C)c1cccc2c1CN(C1CCC(=O)NC1=O)C2=O] | N-(4-((2-(2,6-dioxopiperidin-3-yl)-1- oxoisoindolin-4- yl)(pentyl)amino)butyl)-3,3- dimethylbutanamide |
| 151 | 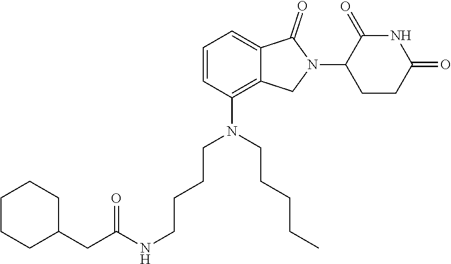 [SMILES: CCCCCN(CCCCNC(=O)CC1CCCCC1)c1cccc2c1CN(C1CCC(=O)NC1=O)C2=O] | 2-cyclohexyl-N-(4-((2-(2,6- dioxopiperidin-3-yl)-1-oxoisoindolin- 4-yl)(pentyl)amino)butyl)acetamide |
| 163 | 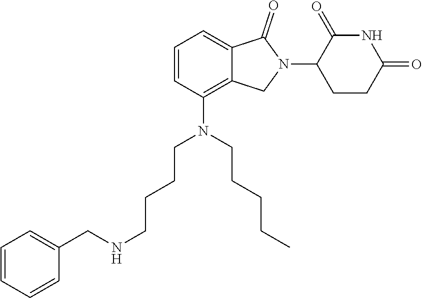 [SMILES: CCCCCN(CCCCNCc1ccccc1)c1cccc2c1CN(C1CCC(=O)NC1=O)C2=O] | 3-(4-((4- (benzylamino)butyl)(pentyl)amino)-1- oxoisoindolin-2-yl)piperidine-2,6- dione |
| 164 | 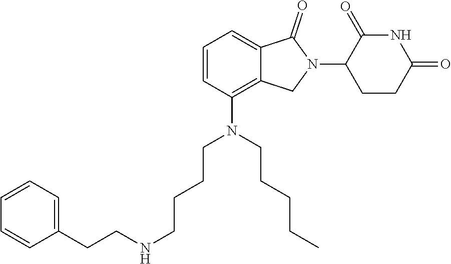 [SMILES: CCCCCN(CCCCNCCc1ccccc1)c1cccc2c1CN(C1CCC(=O)NC1=O)C2=O] | 3-(1-oxo-4-(pentyl(4- (phenethylamino)butyl)amino)isoindolin- 2-yl)piperidine-2,6-dione |
| 165 | 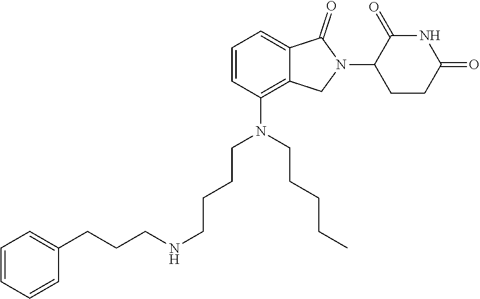 [SMILES: CCCCCN(CCCCNCCCc1ccccc1)c1cccc2c1CN(C1CCC(=O)NC1=O)C2=O] | 3-(1-oxo-4-(pentyl(4-((3- phenylpropyl)amino)butyl)amino) isoindolin-2-yl)piperidine-2,6-dione |
| 166 | 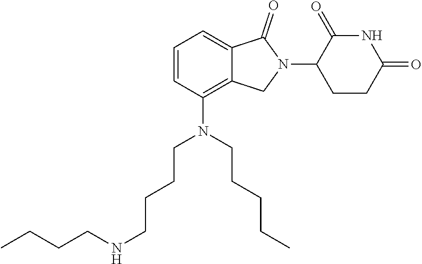 [SMILES: CCCCCN(CCCCNCCCC)c1cccc2c1CN(C1CCC(=O)NC1=O)C2=O] | 3-(4-((4- (butylamino)butyl)(pentyl)amino)-1- oxoisoindolin-2-yl)piperidine-2,6- dione |
| 167 | 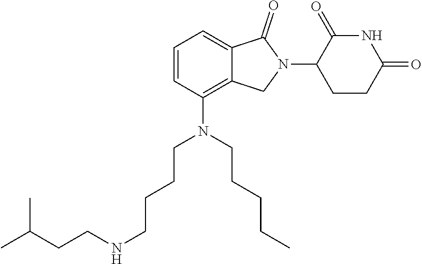 [SMILES: CCCCCN(CCCCNCCC(C)C)c1cccc2c1CN(C1CCC(=O)NC1=O)C2=O] | 3-(4-((4- (isopentylamino)butyl)(pentyl)amino)- 1-oxoisoindolin-2-yl)piperidine-2,6- dione |
| 168 | 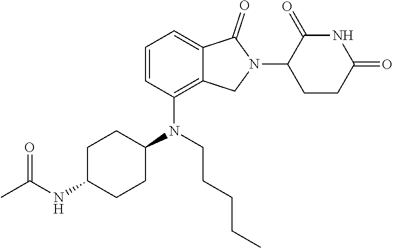 [SMILES: CCCCCN(c1cccc2c1CN(C1CCC(=O)NC1=O)C2=O)[C@H]1CC[C@H](NC(C)=O)CC1] | N-((lr,4r)-4-((2-(2,6-dioxopiperidin-3- yl)-1-oxoisoindolin-4- yl)(pentyl)amino)cyclohexyl) acetamide |
| 169 | 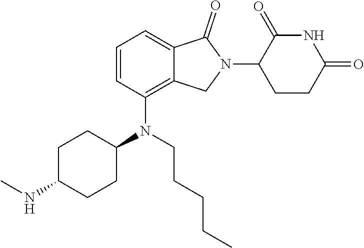 [SMILES: CCCCCN(c1cccc2c1CN(C1CCC(=O)NC1=O)C2=O)[C@H]1CC[C@H](NC)CC1] | 3-(4-(((lr,4r)-4- (methylamino)cyclohexyl)(pentyl)amino)- 1-oxoisoindolin-2-yl)piperidine-2,6- dione |
| 181 | 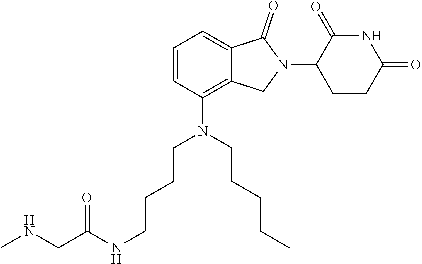 [SMILES: CCCCCN(CCCCNC(=O)CNC)c1cccc2c1CN(C1CCC(=O)NC1=O)C2=O] | N-(4-((2-(2,6-dioxopiperidin-3-yl)-1- oxoisoindolin-4- yl)(pentyl)amino)butyl)-2- (methylamino)acetamide |
| 223 | 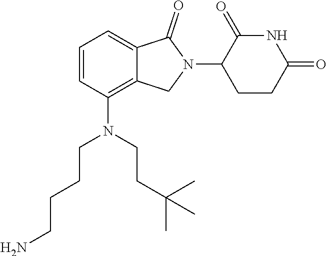 [SMILES: CC(C)(C)CCN(CCCCN)c1cccc2c1CN(C1CCC(=O)NC1=O)C2=O] | 3-(4-((4-aminobutyl)(3,3- dimethylbutyl)amino)-1-oxoisoindolin- 2-yl)piperidine-2,6-dione |
| 235 | 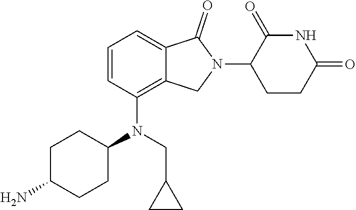 [SMILES: N[C@H]1CC[C@H](N(CC2CC2)c2cccc3c2CN(C2CCC(=O)NC2=O)C3=O)CC1] | 3-(4-(((1r,4r)-4- aminocyclohexyl)(cyclopropylmethyl) amino)-1-oxoisoindolin-2- yl)piperidine-2,6-dione |
| 236 | 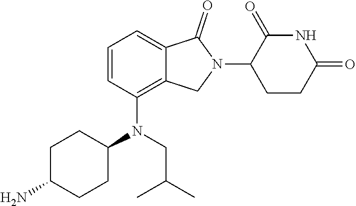 [SMILES: CC(C)CN(c1cccc2c1CN(C1CCC(=O)NC1=O)C2=O)[C@H]1CC[C@H](N)CC1] | 3-(4-(((1r,4r)-4- aminocyclohexyl)(isobutyl)amino)-1- oxoisoindolin-2-yl)piperidine-2,6- dione |
| 237 | 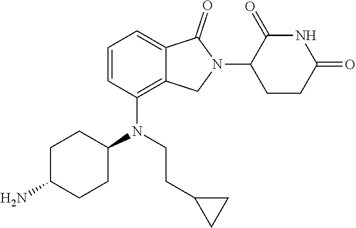 [SMILES: N[C@H]1CC[C@H](N(CCC2CC2)c2cccc3c2CN(C2CCC(=O)NC2=O)C3=O)CC1] | 3-(4-(((1r,4r)-4-aminocyclohexyl)(2- cyclopropylethyl)amino)-1- oxoisoindolin-2-yl)piperidine-2,6- dione |
| 238 | 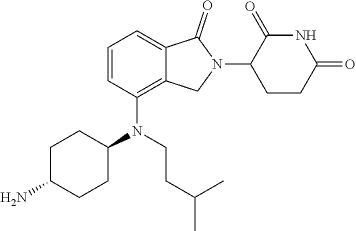 [SMILES: CC(C)CCN(c1cccc2c1CN(C1CCC(=O)NC1=O)C2=O)[C@H]1CC[C@H](N)CC1] | 3-(4-(((lr,4r)-4- aminocyclohexyl)(isopentyl)amino)-1- oxoisoindolin-2-yl)piperidine-2,6- dione |
| 239 | 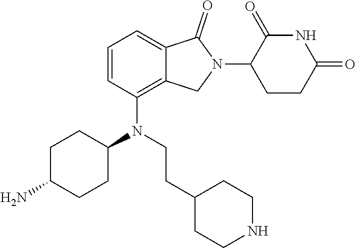 [SMILES: N[C@H]1CC[C@H](N(CCC2CCNCC2)c2cccc3c2CN(C2CCC(=O)NC2=O)C3=O)CC1] | 3-(4-(((1r,4r)-4-aminocyclohexyl)(2- (piperidin-4-yl)ethyl)amino)-1- oxoisoindolin-2-yl)piperidine-2,6- dione |
| 240 | 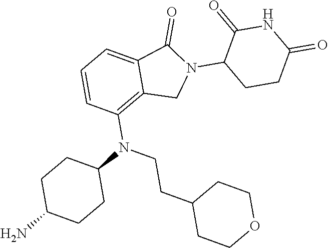 [SMILES: N[C@H]1CC[C@H](N(CCC2CCOCC2)c2cccc3c2CN(C2CCC(=O)NC2=O)C3=O)CC1] | 3-(4-(((1r,4r)-4-aminocyclohexyl)(2- (tetrahydro-2H-pyran-4- yl)ethyl)amino)-1-oxoisoindolin-2- yl)piperidine-2,6-dione |
| 241 | 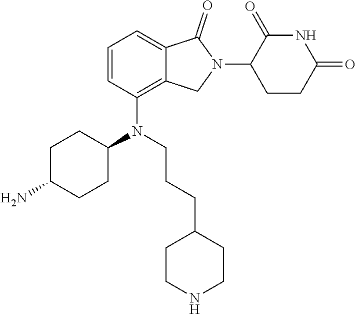 [SMILES: N[C@H]1CC[C@H](N(CCCC2CCNCC2)c2cccc3c2CN(C2CCC(=O)NC2=O)C3=O)CC1] | 3-(4-(((lr,4r)-4-aminocyclohexyl)(3- (piperidin-4-yl)propyl)amino)-1- oxoisoindolin-2-yl)piperidine-2,6- dione |
| 242 | 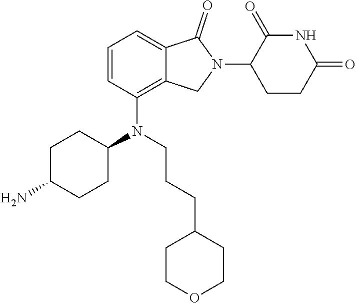 [SMILES: N[C@H]1CC[C@H](N(CCCC2CCOCC2)c2cccc3c2CN(C2CCC(=O)NC2=O)C3=O)CC1] | 3-(4-(((1r,4r)-4-aminocyclohexyl)(3- (tetrahydro-2H-pyran-4- yl)propyl)amino)-1-oxoisoindolin-2- yl)piperidine-2,6-dione |
| 243 | 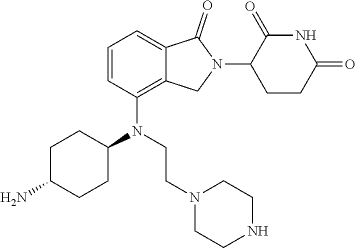 [SMILES: N[C@H]1CC[C@H](N(CCN2CCNCC2)c2cccc3c2CN(C2CCC(=O)NC2=O)C3=O)CC1] | 3-(4-(((1r,4r)-4-aminocyclohexyl)(2- (piperazin-1-yl)ethyl)amino)-1- oxoisoindolin-2-yl)piperidine-2,6- dione |
| 244 | 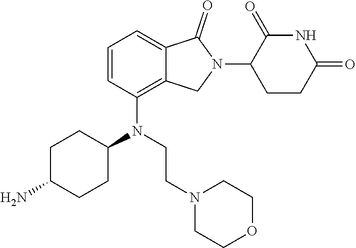 [SMILES: N[C@H]1CC[C@H](N(CCN2CCOCC2)c2cccc3c2CN(C2CCC(=O)NC2=O)C3=O)CC1] | 3-(4-(((1r,4r)-4-aminocyclohexyl)(2- morpholinoethyl)amino)-1- oxoisoindolin-2-yl)piperidine-2,6- dione |
| 245 | 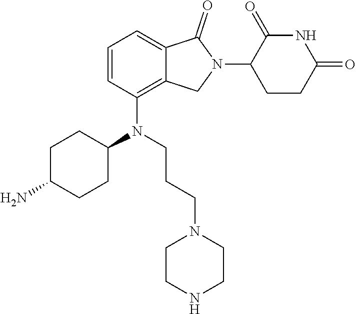 [SMILES: N[C@H]1CC[C@H](N(CCCN2CCNCC2)c2cccc3c2CN(C2CCC(=O)NC2=O)C3=O)CC1] | 3-(4-(((1r,4r)-4-aminocyclohexyl)(3- (piperazin-1-yl)propyl)amino)-1- oxoisoindolin-2-yl)piperidine-2,6- dione |
| 246 | 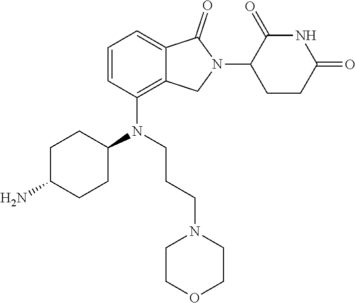 [SMILES: N[C@H]1CC[C@H](N(CCCN2CCOCC2)c2cccc3c2CN(C2CCC(=O)NC2=O)C3=O)CC1] | 3-(4-(((1r,4r)-4-aminocyclohexyl)(3- morpholinopropyl)amino)-1- oxoisoindolin-2-yl)piperidine-2,6- dione |
| 247 | 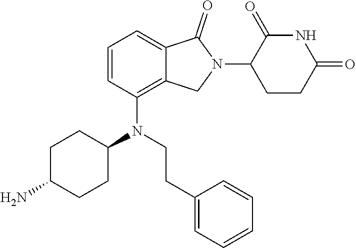 [SMILES: N[C@H]1CC[C@H](N(CCc2ccccc2)c2cccc3c2CN(C2CCC(=O)NC2=O)C3=O)CC1] | 3-(4-(((1r,4r)-4- aminocyclohexyl)(phenethyl)amino)-1- oxoisoindolin-2-yl)piperidine-2,6- dione |
| 251 | 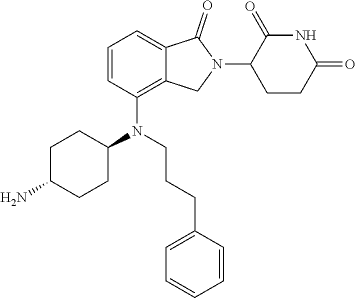 [SMILES: N[C@H]1CC[C@H](N(CCCc2ccccc2)c2cccc3c2CN(C2CCC(=O)NC2=O)C3=O)CC1] | 3-(4-(((1r,4r)-4-aminocyclohexyl)(3- phenylpropyl)amino)-1-oxoisoindolin- 2-yl)piperidine-2,6-dione |
| 255 | 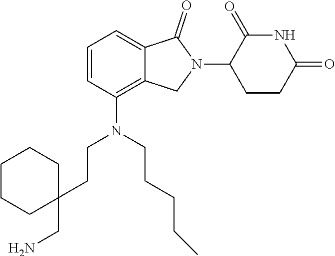 [SMILES: CCCCCN(CCC1(CN)CCCCC1)c1cccc2c1CN(C1CCC(=O)NC1=O)C2=O] | 3-(4-((2-(1- (aminomethyl)cyclohexyl)ethyl)(pentyl) amino)-1-oxoisoindolin-2- yl)piperidine-2,6-dione |
| 267 | 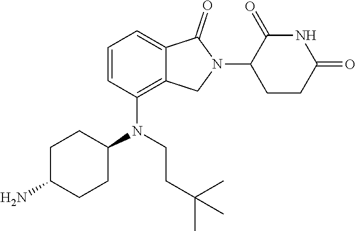 [SMILES: CC(C)(C)CCN(c1cccc2c1CN(C1CCC(=O)NC1=O)C2=O)[C@H]1CC[C@H](N)CC1] | 3-(4-(((1r,4r)-4-aminocyclohexyl)(3,3- dimethylbutyl)amino)-1-oxoisoindolin- 2-yl)piperidine-2,6-dione |
| 268 | 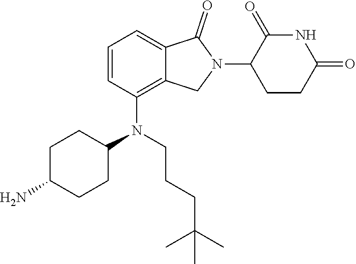 | 3-(4-(((1r,4r)-4-aminocyclohexyl)(4,4- dimethylpentyl)amino)-1- oxoisoindolin-2-yl)piperidine-2,6- dione |
| 273 | 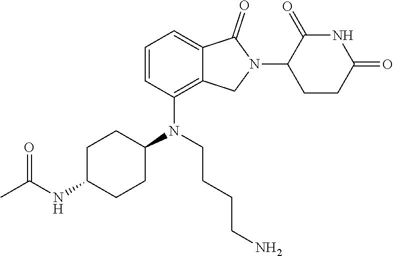 [SMILES: CC(=O)N[C@H]1CC[C@H](N(CCCCN)c2cccc3c2CN(C2CCC(=O)NC2=O)C3=O)CC1] | N-((1r,4r)-4-((4-aminobutyl)(2-(2,6- dioxopiperidin-3-yl)-1-oxoisoindolin- 4-yl)amino)cyclohexyl)acetamide |
| 305 | 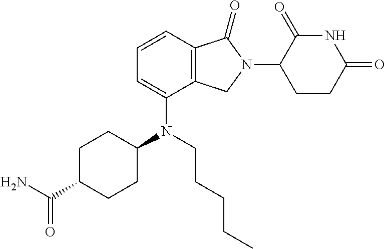 [SMILES: CCCCCN(c1cccc2c1CN(C1CCC(=O)NC1=O)C2=O)[C@H]1CC[C@H](C(N)=O)CC1] | (lr,4r)-4-((2-(2,6-dioxopiperidin-3-yl)- 1-oxoisoindolin-4- yl)(pentyl)amino) cyclohexanecarboxamide |
| 306 | 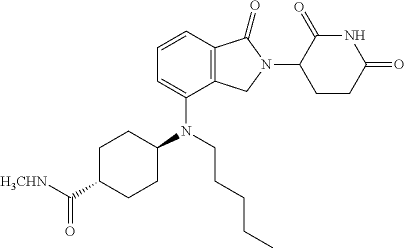 | (lr,4r)-4-((2-(2,6-dioxopiperidin-3-yl)- 1-oxoisoindolin-4-yl)(pentyl)amino)- N-methylcyclohexanecarboxamide |
| 307 | 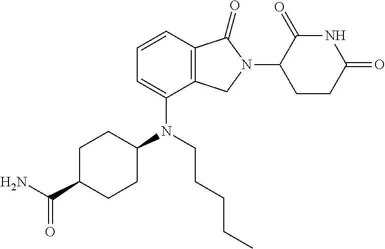 [SMILES: CCCCCN(c1cccc2c1CN(C1CCC(=O)NC1=O)C2=O)[C@H]1CC[C@@H](C(N)=O)CC1] | (1s,4s)-4-((2-(2,6-dioxopiperidin-3-yl)- 1-oxoisoindolin-4- yl)(pentyl)amino) cyclohexanecarboxamide |
| 308 | 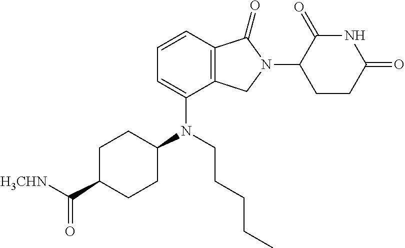 | (1s,4s)-4-((2-(2,6-dioxopiperidin-3-yl)- 1-oxoisoindolin-4-yl)(pentyl)amino)- N-methylcyclohexanecarboxamide |
| 309 | 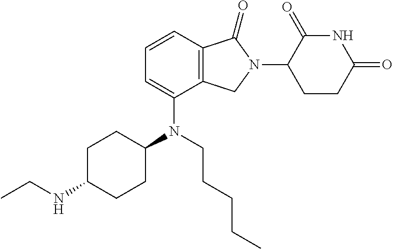 [SMILES: CCCCCN(c1cccc2c1CN(C1CCC(=O)NC1=O)C2=O)[C@H]1CC[C@H](NCC)CC1] | 3-(4-(((1r,4r)-4- (ethylamino)cyclohexyl)(pentyl)amino)- 1-oxoisoindolin-2-yl)piperidine-2,6- dione |
| 310 | 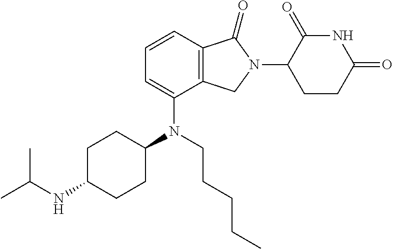 [SMILES: CCCCCN(c1cccc2c1CN(C1CCC(=O)NC1=O)C2=O)[C@H]1CC[C@H](NC(C)C)CC1] | 3-(4-(((lr,4r)-4- (isopropylamino)cyclohexyl)(pentyl) amino)-1-oxoisoindolin-2-yl)piperidine- 2,6-dione |
| 311 | 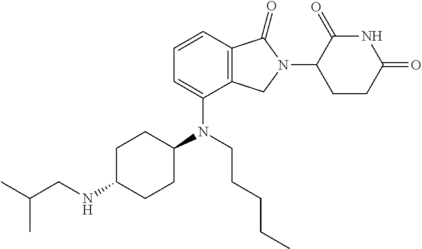 [SMILES: CCCCCN(c1cccc2c1CN(C1CCC(=O)NC1=O)C2=O)[C@H]1CC[C@H](NCC(C)C)CC1] | 3-(4-(((1r,4r)-4- (isobutylamino)cyclohexyl)(pentyl) amino)-1-oxoisoindolin-2-yl)piperidine- 2,6-dione |
| 312 | 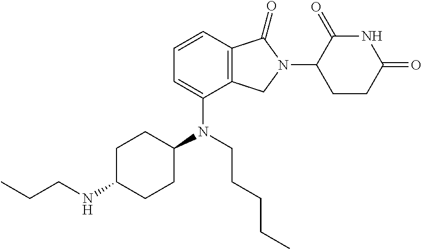 [SMILES: CCCCCN(c1cccc2c1CN(C1CCC(=O)NC1=O)C2=O)[C@H]1CC[C@H](NCCC)CC1] | 3-(1-oxo-4-(pentyl((1r,4r)-4- (propylamino)cyclohexyl)amino) isoindolin-2-yl)piperidine-2,6-dione |
| 313 | 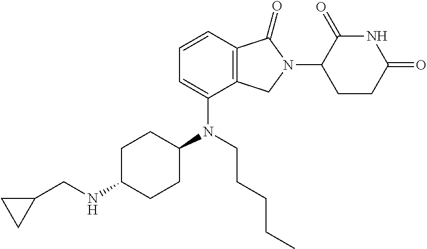 [SMILES: CCCCCN(c1cccc2c1CN(C1CCC(=O)NC1=O)C2=O)[C@H]1CC[C@H](NCC2CC2)CC1] | 3-(4-(((1r,4r)-4- ((cyclopropylmethyl)amino)cyclohexyl) (pentyl)amino)-1-oxoisoindolin-2- yl)piperidine-2,6-dione |
| 314 | 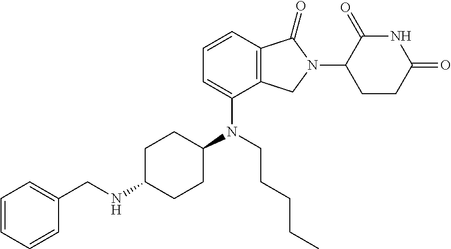 [SMILES: CCCCCN(c1cccc2c1CN(C1CCC(=O)NC1=O)C2=O)[C@H]1CC[C@H](NCc2ccccc2)CC1] | 3-(4-(((1r,4r)-4- (benzylamino)cyclohexyl)(pentyl) amino)-1-oxoisoindolin-2-yl) piperidine-2,6-dione |
| 323 | 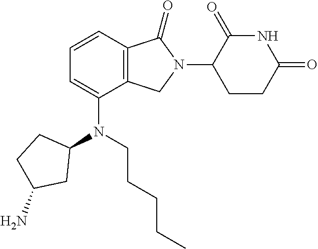 [SMILES: CCCCCN(c1cccc2c1CN(C1CCC(=O)NC1=O)C2=O)[C@@H]1CC[C@@H](N)C1] | 3-(4-(((1R,3R)-3- aminocyclopentyl)(pentyl)amino)-1- oxoisoindolin-2-yl)piperidine-2,6- dione |
| 324 | 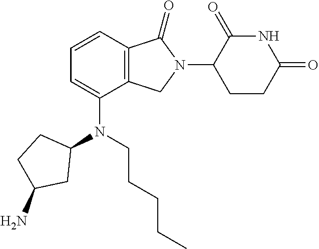 [SMILES: CCCCCN(c1cccc2c1CN(C1CCC(=O)NC1=O)C2=O)[C@@H]1CC[C@H](N)C1] | 3-(4-(((1R,3S)-3- aminocyclopentyl)(pentyl)amino)-1- oxoisoindolin-2-yl)piperidine-2,6- dione |
| 325 | 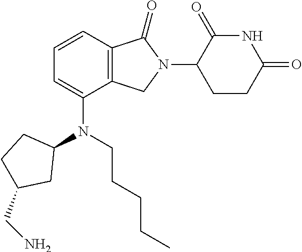 [SMILES: CCCCCN(c1cccc2c1CN(C1CCC(=O)NC1=O)C2=O)[C@@H]1CC[C@@H](CN)C1] | 3-(4-(((1R,3R)-3- (aminomethyl)cyclopentyl)(pentyl) amino)-1-oxoisoindolin-2- yl)piperidine-2,6-dione |
| 325A | 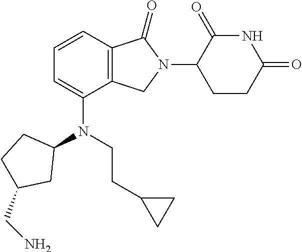 [SMILES: NC[C@@H]1CC[C@@H](N(CCC2CC2)c2cccc3c2CN(C2CCC(=O)NC2=O)C3=O)C1] | 3-(4-(((1R,3R)-3- (aminomethyl)cyclopentyl)(2- cyclopropylethyl)amino)-1- oxoisoindolin-2-yl)piperidine-2,6- dione |
| 326 | 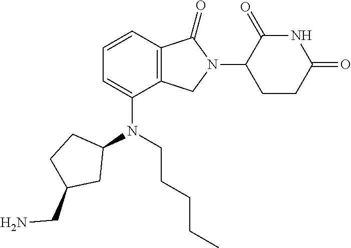 [SMILES: CCCCCN(c1cccc2c1CN(C1CCC(=O)NC1=O)C2=O)[C@@H]1CC[C@H](CN)C1] | 3-(4-(((1R,3S)-3- (aminomethyl)cyclopentyl)(pentyl) amino)-1-oxoisoindolin-2- yl)piperidine-2,6-dione |
| 326A | 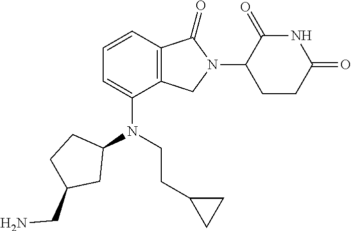 [SMILES: NC[C@H]1CC[C@@H](N(CCC2CC2)c2cccc3c2CN(C2CCC(=O)NC2=O)C3=O)C1] | 3-(4-(((1R,3S)-3- (aminomethyl)cyclopentyl)(2- cyclopropylethyl)amino)-1- oxoisoindolin-2-yl)piperidine-2,6- dione |
| 327 | 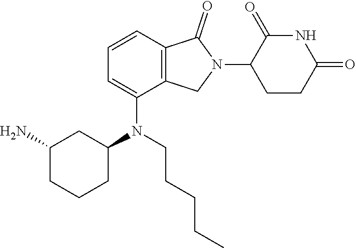 [SMILES: CCCCCN(c1cccc2c1CN(C1CCC(=O)NC1=O)C2=O)[C@H]1CCC[C@H](N)C1] | 3-(4-(((1S,3S)-3- aminocyclohexyl)(pentyl)amino)-1- oxoisoindolin-2-yl)piperidine-2,6- dione |
| 328 | 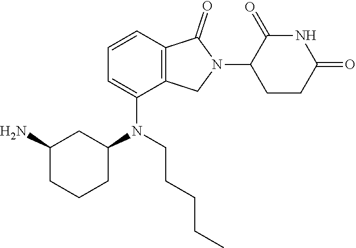 [SMILES: CCCCCN(c1cccc2c1CN(C1CCC(=O)NC1=O)C2=O)[C@H]1CCC[C@@H](N)C1] | 3-(4-(((1S,3R)-3- aminocyclohexyl)(pentyl)amino)-1- oxoisoindolin-2-yl)piperidine-2,6- dione |
| 329 | 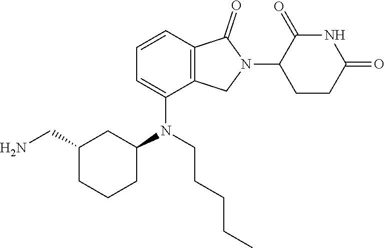 [SMILES: CCCCCN(c1cccc2c1CN(C1CCC(=O)NC1=O)C2=O)[C@H]1CCC[C@H](CN)C1] | 3-(4-(((1S,3S)-3- (aminomethyl)cyclohexyl)(pentyl) amino)-1-oxoisoindolin-2-yl) piperidine-2,6-dione |
| 330 | 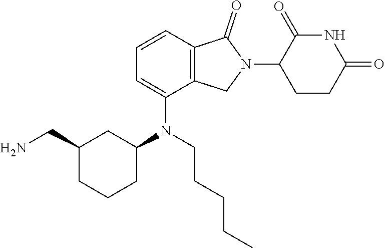 [SMILES: CCCCCN(c1cccc2c1CN(C1CCC(=O)NC1=O)C2=O)[C@H]1CCC[C@@H](CN)C1] | 3-(4-(((1S,3R)-3- (aminomethyl)cyclohexyl)(pentyl) amino)-1-oxoisoindolin-2- yl)piperidine-2,6-dione |
| 331 | 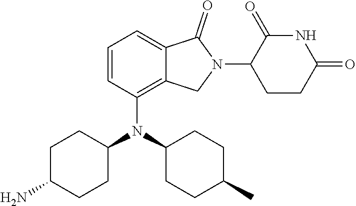 [SMILES: C[C@H]1CC[C@@H](N(c2cccc3c2CN(C2CCC(=O)NC2=O)C3=O)[C@H]2CC[C@H](N)CC2)CC1] | 3-(4-(((1r,4R)-4- aminocyclohexyl)((1s,4S)-4- methylcyclohexyl)amino)-1- oxoisoindolin-2-yl)piperidine-2,6- dione |
| 332 | 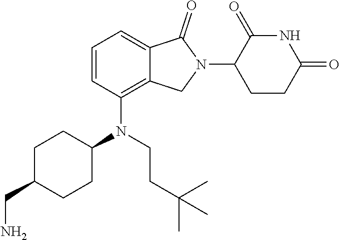 [SMILES: CC(C)(C)CCN(c1cccc2c1CN(C1CCC(=O)NC1=O)C2=O)[C@H]1CC[C@@H](CN)CC1] | 3-(4-(((1s,4s)-4- (aminomethyl)cyclohexyl)(3,3- dimethylbutyl)amino)-1-oxoisoindolin- 2-yl)piperidine-2,6-dione |
| 333 | 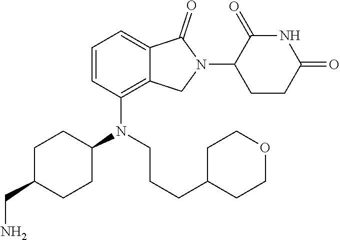 [SMILES: NC[C@H]1CC[C@@H](N(CCCC2CCOCC2)c2cccc3c2CN(C2CCC(=O)NC2=O)C3=O)CC1] | 3-(4-(((1s,4s)-4- (aminomethyl)cyclohexyl)(3- (tetrahydro-2H-pyran-4- yl)propyl)amino)-1-oxoisoindolin-2- yl)piperidine-2,6-dione |
| 334 | 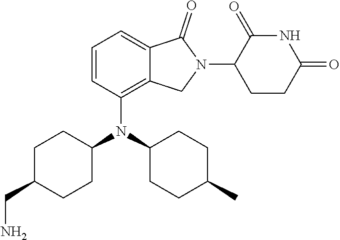 [SMILES: C[C@H]1CC[C@@H](N(c2cccc3c2CN(C2CCC(=O)NC2=O)C3=O)[C@H]2CC[C@@H](CN)CC2)CC1] | 3-(4-(((1s,4S)-4- (aminomethyl)cyclohexyl)((1s,4S)-4- methylcyclohexyl)amino)-1- oxoisoindolin-2-yl)piperidine-2,6- dione |
| 340 | 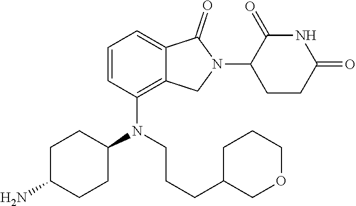 [SMILES: N[C@H]1CC[C@H](N(CCCC2CCCOC2)c2cccc3c2CN(C2CCC(=O)NC2=O)C3=O)CC1] | 3-(4-(((1r,4r)-4-aminocyclohexyl)(3- (tetrahydro-2H-pyran-3- yl)propyl)amino)-1-oxoisoindolin-2- yl)piperidine-2,6-dione |
| 341 | 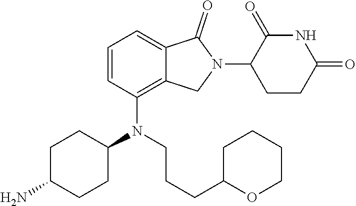 [SMILES: N[C@H]1CC[C@H](N(CCCC2CCCCO2)c2cccc3c2CN(C2CCC(=O)NC2=O)C3=O)CC1] | 3-(4-(((1r,4r)-4-aminocyclohexyl)(3- (tetrahydro-2H-pyran-2- yl)propyl)amino)-1-oxoisoindolin-2- yl)piperidine-2,6-dione |
| 344 | 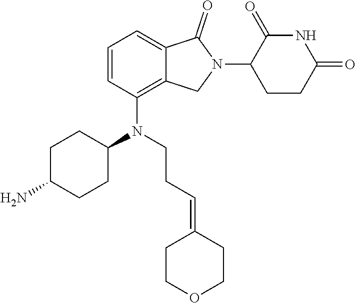 [SMILES: N[C@H]1CC[C@H](N(CCC=C2CCOCC2)c2cccc3c2CN(C2CCC(=O)NC2=O)C3=O)CC1] | 3-(4-(((1r,4r)-4-aminocyclohexyl)(3- (dihydro-2H-pyran-4(3H)- ylidene)propyl)amino)-1- oxoisoindolin-2-yl)piperidine-2,6- dione |
| 345 | 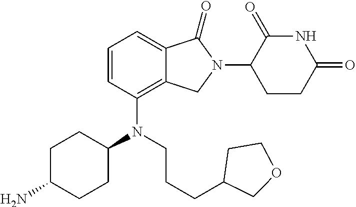 [SMILES: N[C@H]1CC[C@H](N(CCCC2CCOC2)c2cccc3c2CN(C2CCC(=O)NC2=O)C3=O)CC1] | 3-(4-(((1r,4r)-4-aminocyclohexyl)(3- (tetrahydrofuran-3-yl)propyl)amino)-1- oxoisoindolin-2-yl)piperidine-2,6- dione |
| 346 | 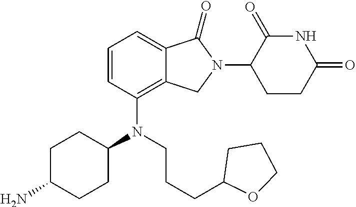 [SMILES: N[C@H]1CC[C@H](N(CCCC2CCCO2)c2cccc3c2CN(C2CCC(=O)NC2=O)C3=O)CC1] | 3-(4-(((1r,4r)-4-aminocyclohexyl)(3- (tetrahydrofuran-2-yl)propyl)amino)-1- oxoisoindolin-2-yl)piperidine-2,6- dione |
| 347 | 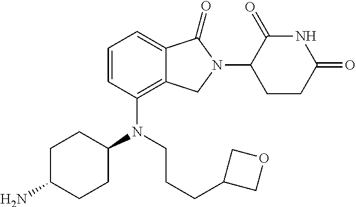 [SMILES: N[C@H]1CC[C@H](N(CCCC2COC2)c2cccc3c2CN(C2CCC(=O)NC2=O)C3=O)CC1] | 3-(4-(((1r,4r)-4-aminocyclohexyl)(3- (oxetan-3-yl)propyl)amino)-1- oxoisoindolin-2-yl)piperidine-2,6- dione |
| 349 | 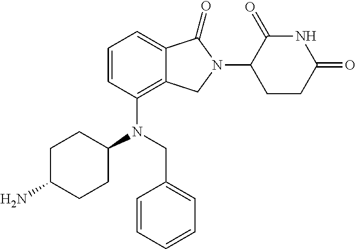 [SMILES: N[C@H]1CC[C@H](N(Cc2ccccc2)c2cccc3c2CN(C2CCC(=O)NC2=O)C3=O)CC1] | 3-(4-(((1r,4r)-4- aminocyclohexyl)(benzyl)amino)-1- oxoisoindolin-2-yl)piperidine-2,6- dione |
| 352 | 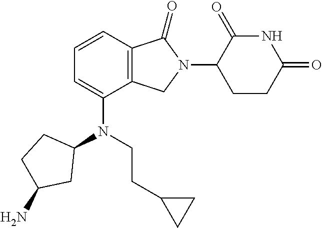 [SMILES: N[C@H]1CC[C@@H](N(CCC2CC2)c2cccc3c2CN(C2CCC(=O)NC2=O)C3=O)C1] | 3-(4-(((1R,3S)-3-aminocyclopentyl)(2- cyclopropylethyl)amino)-1- oxoisoindolin-2-yl)piperidine-2,6- dione |
| 353 | 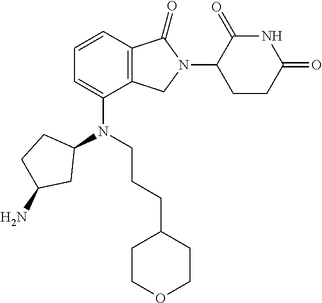 [SMILES: N[C@H]1CC[C@@H](N(CCCC2CCOCC2)c2cccc3c2CN(C2CCC(=O)NC2=O)C3=O)C1] | 3-(4-(((1R,3S)-3-aminocyclopentyl)(3- (tetrahydro-2H-pyran-4- yl)propyl)amino)-1-oxoisoindolin-2- yl)piperidine-2,6-dione |
| 354 | 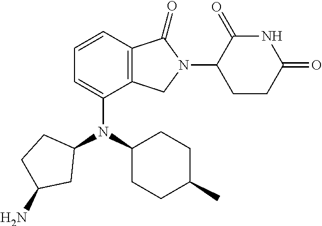 [SMILES: C[C@H]1CC[C@@H](N(c2cccc3c2CN(C2CCC(=O)NC2=O)C3=O)[C@@H]2CC[C@H](N)C2)CC1] | 3-(4-(((1R,3S)-3- aminocyclopentyl)((1s,4S)-4- methylcyclohexyl)amino)-1- oxoisoindolin-2-yl)piperidine-2,6- dione |
| 355 | 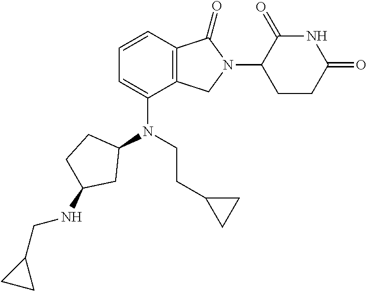 [SMILES: O=C1CCC(N2Cc3c(cccc3N(CCC3CC3)[C@@H]3CC[C@H](NCC4CC4)C3)C2=O)C(=O)N1] | 3-(4-((2-cyclopropylethyl)((1R,3S)-3- ((cyclopropylmethyl)amino)cyclopenty 1)amino)-1-oxoisoindolin-2- yl)piperidine-2,6-dione |
| 356 | 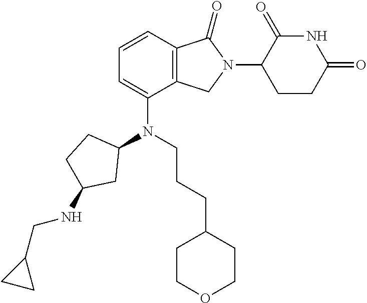 [SMILES: O=C1CCC(N2Cc3c(cccc3N(CCCC3CCOCC3)[C@@H]3CC[C@H](NCC4CC4)C3)C2=O)C(=O)N1] | 3-(4-(((1R,3S)-3- ((cyclopropylmethyl)amino)cyclopenty 1)(3-(tetrahydro-2H-pyran-4- yl)propyl)amino)-1-oxoisoindolin-2- yl)piperidine-2,6-dione |
| 358 | 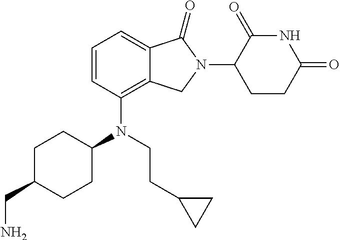 [SMILES: NC[C@H]1CC[C@@H](N(CCC2CC2)c2cccc3c2CN(C2CCC(=O)NC2=O)C3=O)CC1] | 3-(4-(((1s,4s)-4- (aminomethyl)cyclohexyl)(2- cyclopropylethyl)amino)-1- oxoisoindolin-2-yl)piperidine-2,6- dione |
| 359 | 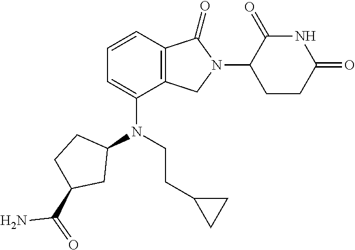 [SMILES: NC(=O)[C@H]1CC[C@@H](N(CCC2CC2)c2cccc3c2CN(C2CCC(=O)NC2=O)C3=O)C1] | (1S,3R)-3-((2-cyclopropylethyl)(2- (2,6-dioxopiperidin-3-yl)-1- oxoisoindolin-4- yl)amino)cyclopentanecarboxamide |
| 360 | 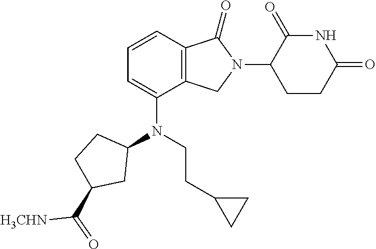 | (1S,3R)-3-((2-cyclopropylethyl)(2- (2,6-dioxopiperidin-3-yl)-1- oxoisoindolin-4-yl)amino)-N- methylcyclopentanecarboxamide |
| 361 | 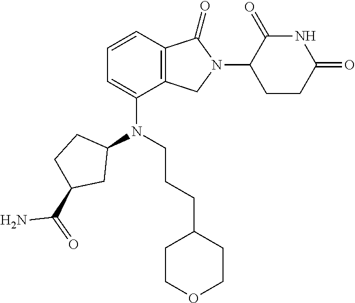 [SMILES: NC(=O)[C@H]1CC[C@@H](N(CCCC2CCOCC2)c2cccc3c2CN(C2CCC(=O)NC2=O)C3=O)C1] | (1S,3R)-3-((2-(2,6-dioxopiperidin-3- yl)-1-oxoisoindolin-4-yl)(3- (tetrahydro-2H-pyran-4- yl)propyl)amino) cyclopentanecarboxamide |
| 362 | 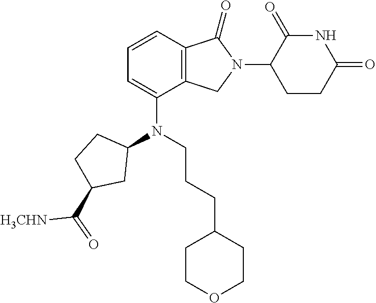 | (1S,3R)-3-((2-(2,6-dioxopiperidin-3- yl)-1-oxoisoindolin-4-yl)(3- (tetrahydro-2H-pyran-4- yl)propyl)amino)-N- methylcyclopentanecarboxamide |
| 512 | 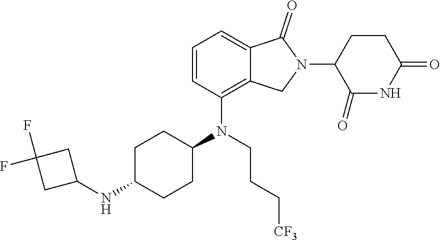 [SMILES: O=C1CCC(N2Cc3c(cccc3N(CCCC(F)(F)F)[C@H]3CC[C@H](NC4CC(F)(F)C4)CC3)C2=O)C(=O)N1] | 3-(4-(((1r,4r)-4-((3,3- difluorocyclobutyl)amino)cyclohexyl) (4,4,4-trifluorobutyl)amino)-1- oxoisoindolin-2-yl)piperidine-2,6- dione |
| 513 | 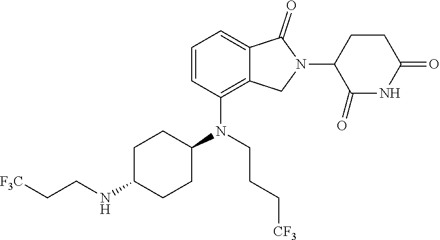 [SMILES: O=C1CCC(N2Cc3c(cccc3N(CCCC(F)(F)F)[C@H]3CC[C@H](NCCC(F)(F)F)CC3)C2=O)C(=O)N1] | 3-(1-oxo-4-((4,4,4- trifluorobutyl)((lr,4r)-4-((3,3,3- trifluoropropyl)amino)cyclohexyl)amin o)isoindolin-2-yl)piperidine-2,6-dione |
| 514 | 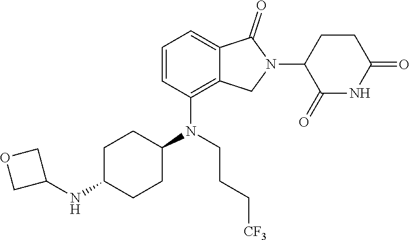 [SMILES: O=C1CCC(N2Cc3c(cccc3N(CCCC(F)(F)F)[C@H]3CC[C@H](NC4COC4)CC3)C2=O)C(=O)N1] | 3-(4-(((1r,4r)-4-(oxetan-3- ylamino)cyclohexyl)(4,4,4- trifluorobutyl)amino)-1-oxoisoindolin- 2-yl)piperidine-2,6-dione |
| 515 | 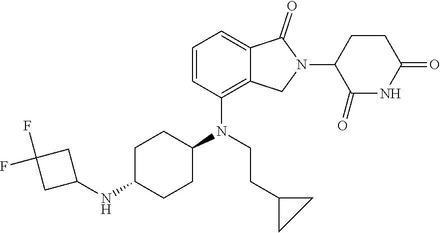 [SMILES: O=C1CCC(N2Cc3c(cccc3N(CCC3CC3)[C@H]3CC[C@H](NC4CC(F)(F)C4)CC3)C2=O)C(=O)N1] | 3-(4-((2-cyclopropylethyl)((1r,4r)-4- ((3,3- difluorocyclobutyl)amino)cyclohexyl) amino)-1-oxoisoindolin-2-yl)piperidine- 2,6-dione |
| 515A | 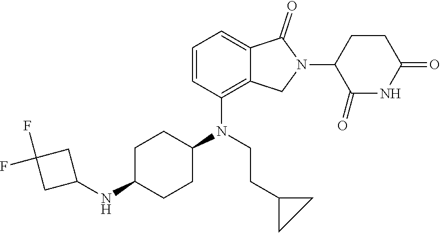 [SMILES: O=C1CCC(N2Cc3c(cccc3N(CCC3CC3)[C@H]3CC[C@@H](NC4CC(F)(F)C4)CC3)C2=O)C(=O)N1] | 3-(4-((2-cyclopropylethyl)((1s,4s)-4- ((3,3- difluorocyclobutyl)amino)cyclohexyl) amino)-1-oxoisoindolin-2- yl)piperidine-2,6-dione |
| 516 | 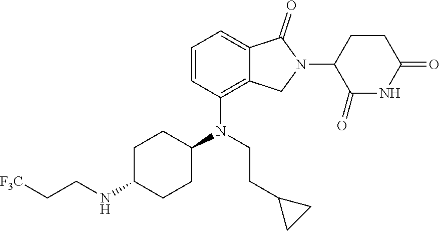 [SMILES: O=C1CCC(N2Cc3c(cccc3N(CCC3CC3)[C@H]3CC[C@H](NCCC(F)(F)F)CC3)C2=O)C(=O)N1] | 3-(4-((2-cyclopropylethyl)((lr,4r)-4- ((3,3,3- trifluoropropyl)amino)cyclohexyl) amino)-1-oxoisoindolin-2-yl) piperidine-2,6-dione |
| 517 | 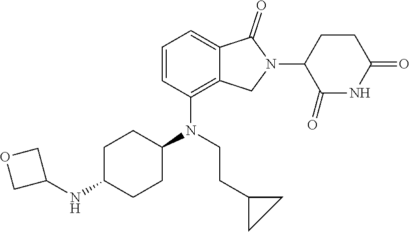 [SMILES: O=C1CCC(N2Cc3c(cccc3N(CCC3CC3)[C@H]3CC[C@H](NC4COC4)CC3)C2=O)C(=O)N1] | 3-(4-((2-cyclopropylethyl)((lr,4r)-4- (oxetan-3-ylamino)cyclohexyl)amino)- 1-oxoisoindolin-2-yl)piperidine-2,6- dione |
| 518 | 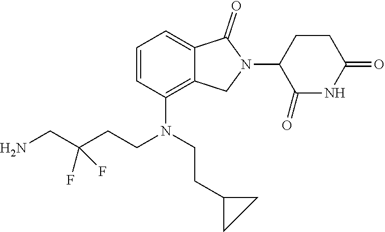 [SMILES: NCC(F)(F)CCN(CCC1CC1)c1cccc2c1CN(C1CCC(=O)NC1=O)C2=O] | 3-(4-((4-amino-3,3-difluorobutyl)(2- cyclopropylethyl)amino)-1- oxoisoindolin-2-yl)piperidine-2,6- dione |
| 519 | 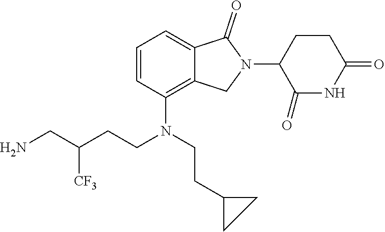 [SMILES: NCC(CCN(CCC1CC1)c1cccc2c1CN(C1CCC(=O)NC1=O)C2=O)C(F)(F)F] | 3-(4-((3-(aminomethyl)-4,4,4- trifluorobutyl)(2- cyclopropylethyl)amino)-1- oxoisoindolin-2-yl)piperidine-2,6- dione |
| 521 | 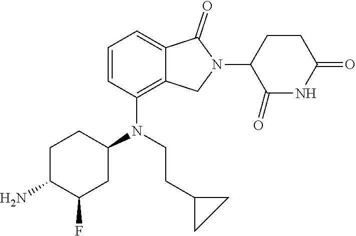 [SMILES: N[C@@H]1CC[C@@H](N(CCC2CC2)c2cccc3c2CN(C2CCC(=O)NC2=O)C3=O)C[C@H]1F] | 3-(4-(((1R,3R,4R)-4-amino-3- fluorocyclohexyl)(2- cyclopropylethyl)amino)-1- oxoisoindolin-2-yl)piperidine-2,6- dione |
| 522 | 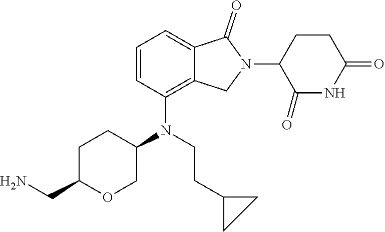 [SMILES: NC[C@H]1CC[C@@H](N(CCC2CC2)c2cccc3c2CN(C2CCC(=O)NC2=O)C3=O)CO1] | 3-(4-(((3R,6R)-6- (aminomethyl)tetrahydro-2H-pyran-3- yl)(2-cyclopropylethyl)amino)-1- oxoisoindolin-2-yl)piperidine-2,6- dione |
| 523 | 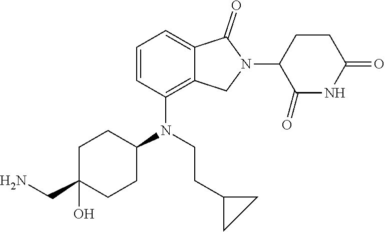 [SMILES: NC[C@]1(O)CC[C@H](N(CCC2CC2)c2cccc3c2CN(C2CCC(=O)NC2=O)C3=O)CC1] | 3-(4-(((1r,4r)-4-(aminomethyl)-4- hydroxycyclohexyl)(2- cyclopropylethyl)amino)-1- oxoisoindolin-2-yl)piperidine-2,6- dione |
| 530 | 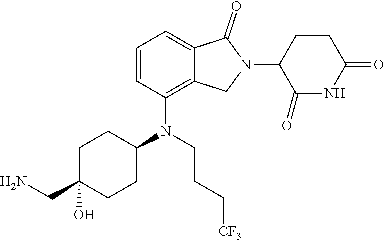 [SMILES: NC[C@]1(O)CC[C@H](N(CCCC(F)(F)F)c2cccc3c2CN(C2CCC(=O)NC2=O)C3=O)CC1] | 3-(4-(((1r,4r)-4-(aminomethyl)-4- hydroxycyclohexyl)(4,4,4- trifluorobutyl)amino)-1-oxoisoindolin- 2-yl)piperidine-2,6-dione |
| 532 | 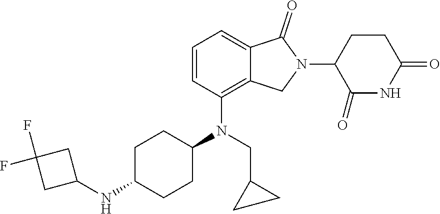 [SMILES: O=C1CCC(N2Cc3c(cccc3N(CC3CC3)[C@H]3CC[C@H](NC4CC(F)(F)C4)CC3)C2=O)C(=O)N1] | 3-(4-((cyclopropylmethyl)((lr,4r)-4- ((3,3- difluorocyclobutyl)amino)cyclohexyl) amino)-1-oxoisoindolin-2- yl)piperidine-2,6-dione |
| 533 | 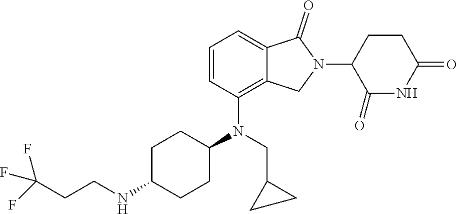 [SMILES: O=C1CCC(N2Cc3c(cccc3N(CC3CC3)[C@H]3CC[C@H](NCCC(F)(F)F)CC3)C2=O)C(=O)N1] | 3-(4-((cyclopropylmethyl)((lr,4r)-4- ((3,3,3- trifluoropropyl)amino)cyclohexyl) amino)-1-oxoisoindolin-2- yl)piperidine-2,6-dione |
| 534 | 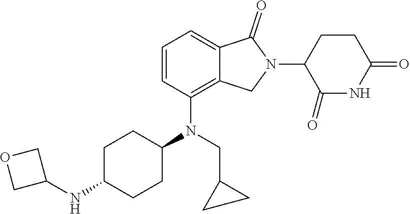 [SMILES: O=C1CCC(N2Cc3c(cccc3N(CC3CC3)[C@H]3CC[C@H](NC4COC4)CC3)C2=O)C(=O)N1] | 3-(4-((cyclopropylmethyl)((1r,4r)-4- (oxetan-3-ylamino)cyclohexyl)amino)- 1-oxoisoindolin-2-yl)piperidine-2,6- dione |
| 535 | 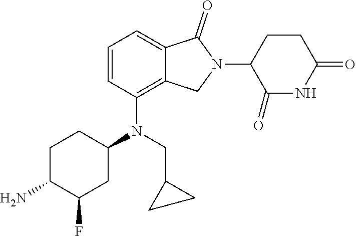 [SMILES: N[C@@H]1CC[C@@H](N(CC2CC2)c2cccc3c2CN(C2CCC(=O)NC2=O)C3=O)C[C@H]1F] | 3-(4-(((1R,3R,4R)-4-amino-3- fluorocyclohexyl)(cyclopropylmethyl) amino)-1-oxoisoindolin-2-yl)piperidine- 2,6-dione |
| 536 | 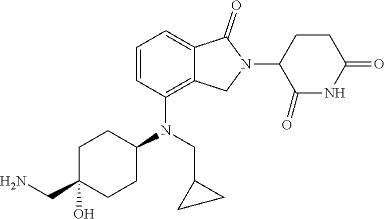 [SMILES: NC[C@]1(O)CC[C@H](N(CC2CC2)c2cccc3c2CN(C2CCC(=O)NC2=O)C3=O)CC1] | 3-(4-(((1r,4r)-4-(aminomethyl)-4- hydroxycyclohexyl)(cyclopropyl methyl)amino)-1-oxoisoindolin-2- yl)piperidine-2,6-dione |
| 539 | 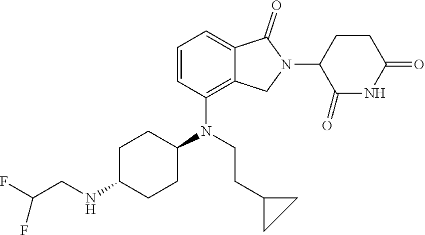 [SMILES: O=C1CCC(N2Cc3c(cccc3N(CCC3CC3)[C@H]3CC[C@H](NCC(F)F)CC3)C2=O)C(=O)N1] | 3-(4-((2-cyclopropylethyl)((lr,4r)-4- ((2,2- difluoroethyl)amino)cyclohexyl) amino)-1-oxoisoindolin-2- yl)piperidine-2,6-dione |
| 539A | 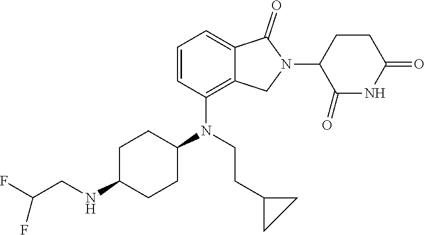 [SMILES: O=C1CCC(N2Cc3c(cccc3N(CCC3CC3)[C@H]3CC[C@@H](NCC(F)F)CC3)C2=O)C(=O)N1] | 3-(4-((2-cyclopropylethyl)((1s,4s)-4- ((2,2- difluoroethyl)amino)cyclohexyl)amino)- 1-oxoisoindolin-2-yl)piperidine-2,6- dione |
| 540 | 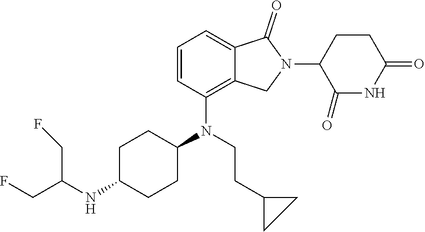 [SMILES: O=C1CCC(N2Cc3c(cccc3N(CCC3CC3)[C@H]3CC[C@H](NC(CF)CF)CC3)C2=O)C(=O)N1] | 3-(4-((2-cyclopropylethyl)((lr,4r)-4- ((1,3-difluoropropan-2- yl)amino)cyclohexyl)amino)-1- oxoisoindolin-2-yl)piperidine-2,6- dione |
| 541 | 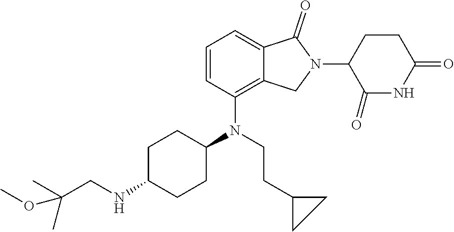 [SMILES: COC(C)(C)CN[C@H]1CC[C@H](N(CCC2CC2)c2cccc3c2CN(C2CCC(=O)NC2=O)C3=O)CC1] | 3-(4-((2-cyclopropylethyl)((lr,4r)-4- ((2-methoxy-2- methylpropyl)amino)cyclohexyl) amino)-1-oxoisoindolin-2- yl)piperidine-2,6-dione |
| 541A | 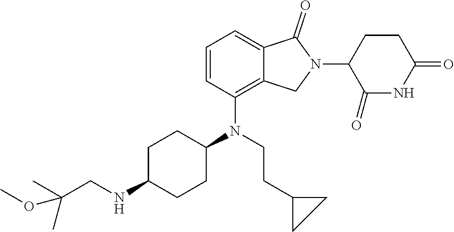 [SMILES: COC(C)(C)CN[C@H]1CC[C@@H](N(CCC2CC2)c2cccc3c2CN(C2CCC(=O)NC2=O)C3=O)CC1] | 3-(4-((2-cyclopropylethyl)((1s,4s)-4- ((2-methoxy-2- methylpropyl)amino)cyclohexyl) amino)-1-oxoisoindolin-2- yl)piperidine-2,6-dione |
| 542 | 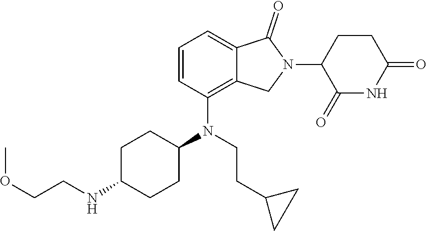 [SMILES: COCCN[C@H]1CC[C@H](N(CCC2CC2)c2cccc3c2CN(C2CCC(=O)NC2=O)C3=O)CC1] | 3-(4-((2-cyclopropylethyl)((lr,4r)-4- ((2- methoxyethyl)amino)cyclohexyl) amino)-1-oxoisoindolin-2- yl)piperidine-2,6-dione |
| 543 | 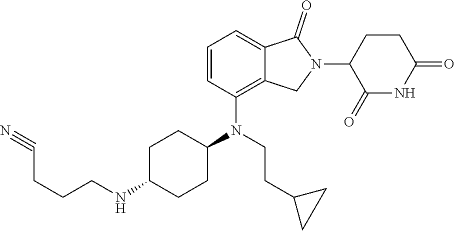 [SMILES: N#CCCCN[C@H]1CC[C@H](N(CCC2CC2)c2cccc3c2CN(C2CCC(=O)NC2=O)C3=O)CC1] | 4-(((1r,4r)-4-((2-cyclopropylethyl)(2- (2,6-dioxopiperidin-3-yl)-1- oxoisoindolin-4- yl)amino)cyclohexyl)amino) butanenitrile |
| 546 | 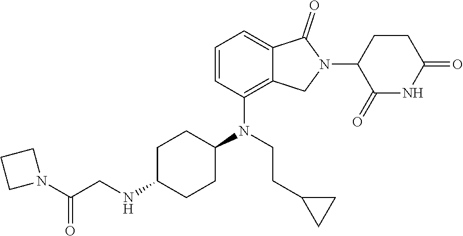 [SMILES: O=C1CCC(N2Cc3c(cccc3N(CCC3CC3)[C@H]3CC[C@H](NCC(=O)N4CCC4)CC3)C2=O)C(=O)N1] | 3-(4-(((1r,4r)-4-((2-(azetidin-1-yl)-2- oxoethyl)amino)cyclohexyl)(2- cyclopropylethyl)amino)-1- oxoisoindolin-2-yl)piperidine-2,6- dione |
| 547 | 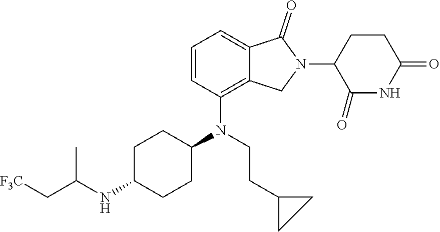 [SMILES: CC(CC(F)(F)F)N[C@H]1CC[C@H](N(CCC2CC2)c2cccc3c2CN(C2CCC(=O)NC2=O)C3=O)CC1] | 3-(4-((2-cyclopropylethyl)((lr,4r)-4- ((4,4,4-trifluorobutan-2- yl)amino)cyclohexyl)amino)-1- oxoisoindolin-2-yl)piperidine-2,6- dione |
| 548 | 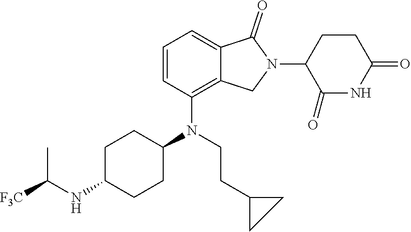 [SMILES: C[C@@H](N[C@H]1CC[C@H](N(CCC2CC2)c2cccc3c2CN(C2CCC(=O)NC2=O)C3=O)CC1)C(F)(F)F] | 3-(4-((2-cyclopropylethyl)((1R,4r)-4- (((R)-1,1,1-trifluoropropan-2- yl)amino)cyclohexyl)amino)-1- oxoisoindolin-2-yl)piperidine-2,6- dione |
| 548A | 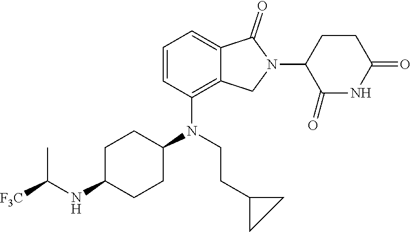 [SMILES: C[C@@H](N[C@H]1CC[C@@H](N(CCC2CC2)c2cccc3c2CN(C2CCC(=O)NC2=O)C3=O)CC1)C(F)(F)F] | 3-(4-((2-cyclopropylethyl)((1S,4s)-4- (((R)-1,1,1-trifluoropropan-2- yl)amino)cyclohexyl)amino)-1- oxoisoindolin-2-yl)piperidine-2,6- dione |
| 549 | 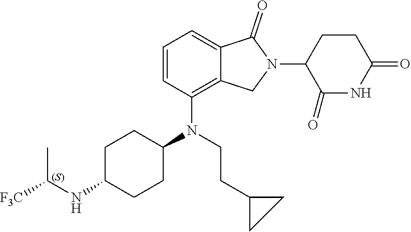 | 3-(4-((2-cyclopropylethyl)((1S,4r)-4- (((S)-1,1,1-trifluoropropan-2- yl)amino)cyclohexyl)amino)-1- oxoisoindolin-2-yl)piperidine-2,6- dione |
| 550 | 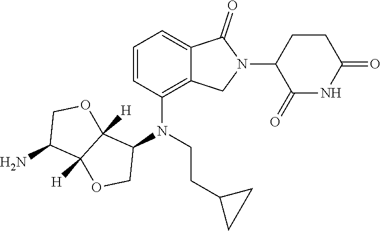 [SMILES: N[C@H]1CO[C@H]2[C@@H]1OC[C@@H]2N(CCC1CC1)c1cccc2c1CN(C1CCC(=O)NC1=O)C2=O] | 3-(4-(((3S,3aR,6S,6aR)-6- aminohexahydrofuro[3,2-b]furan-3- yl)(2-cyclopropylethyl)amino)-1- oxoisoindolin-2-yl)piperidine-2,6- dione |
| 552 | 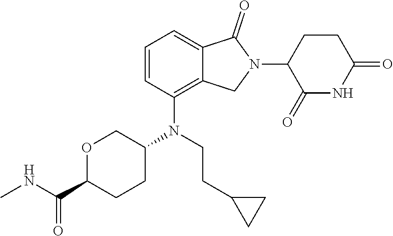 [SMILES: CNC(=O)[C@@H]1CC[C@@H](N(CCC2CC2)c2cccc3c2CN(C2CCC(=O)NC2=O)C3=O)CO1] | (2S,5R)-5-((2-cyclopropylethyl)(2- (2,6-dioxopiperidin-3-yl)-1- oxoisoindolin-4-yl)amino)-N- methyltetrahydro-2H-pyran-2- carboxamide |
| 554 | 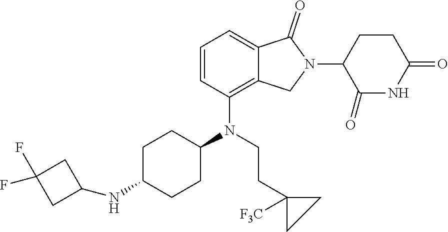 [SMILES: O=C1CCC(N2Cc3c(cccc3N(CCC3(C(F)(F)F)CC3)[C@H]3CC[C@H](NC4CC(F)(F)C4)CC3)C2=O)C(=O)N1] | 3-(4-(((1r,4r)-4-((3,3- difluorocyclobutyl)amino)cyclohexyl) (2-(1- (trifluoromethyl)cyclopropyl)ethyl) amino)-1-oxoisoindolin-2- yl)piperidine-2,6-dione |
| 554A | 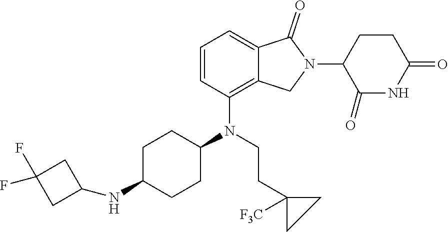 [SMILES: O=C1CCC(N2Cc3c(cccc3N(CCC3(C(F)(F)F)CC3)[C@H]3CC[C@@H](NC4CC(F)(F)C4)CC3)C2=O)C(=O)N1] | 3-(4-(((1s,4s)-4-((3,3- difluorocyclobutyl)amino)cyclohexyl) (2-(1- (trifluoromethyl)cyclopropyl)ethyl) amino)-1-oxoisoindolin-2- yl)piperidine-2,6-dione |
| 557 | 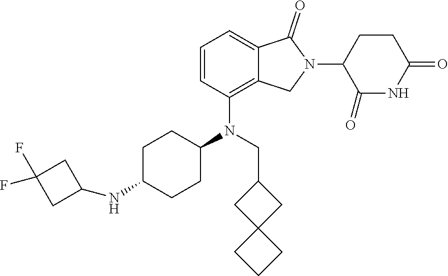 [SMILES: O=C1CCC(N2Cc3c(cccc3N(CC3CC4(CCC4)C3)[C@H]3CC[C@H](NC4CC(F)(F)C4)CC3)C2=O)C(=O)N1] | 3-(4-(((1r,4r)-4-((3,3- difluorocyclobutyl)amino)cyclohexyl) (spiro[3.3]heptan-2-ylmethyl)amino)-1- oxoisoindolin-2-yl)piperidine-2,6- dione |
| 558-2 | 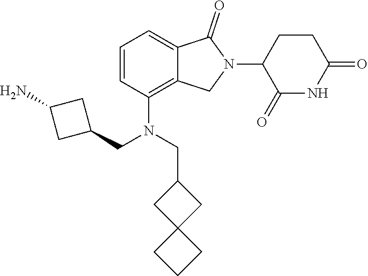 [SMILES: N[C@H]1C[C@H](CN(CC2CC3(CCC3)C2)c2cccc3c2CN(C2CCC(=O)NC2=O)C3=O)C1] | 3-(4-((((1r,3r)-3- aminocyclobutyl)methyl)(spiro[3.3] heptan-2-ylmethyl)amino)-1- oxoisoindolin-2-yl)piperidine-2,6- dione |
| 561 | 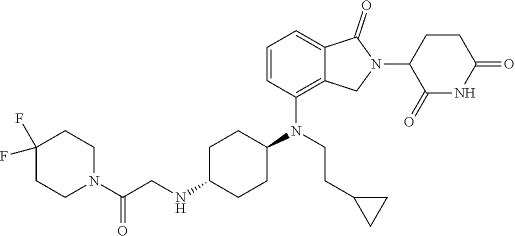 [SMILES: O=C1CCC(N2Cc3c(cccc3N(CCC3CC3)[C@H]3CC[C@H](NCC(=O)N4CCC(F)(F)CC4)CC3)C2=O)C(=O)N1] | 3-(4-((2-cyclopropylethyl)((1r,4r)-4- ((2-(4,4-difluoropiperidin-1-yl)-2- oxoethyl)amino)cyclohexyl)amino)-1- oxoisoindolin-2-yl)piperidine-2,6- dione |
| 562 | 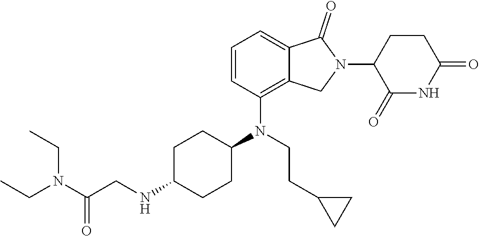 [SMILES: CCN(CC)C(=O)CN[C@H]1CC[C@H](N(CCC2CC2)c2cccc3c2CN(C2CCC(=O)NC2=O)C3=O)CC1] | 2-(((1r,4r)-4-((2-cyclopropylethyl)(2- (2,6-dioxopiperidin-3-yl)-1- oxoisoindolin-4- yl)amino)cyclohexyl)amino)-N,N- diethylacetamide |
| 573 | 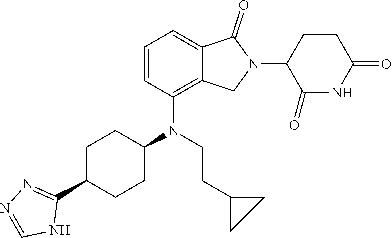 [SMILES: O=C1CCC(N2Cc3c(cccc3N(CCC3CC3)[C@H]3CC[C@@H](c4nnc[nH]4)CC3)C2=O)C(=O)N1] | 3-(4-(((1s,4s)-4-(4H-1,2,4-triazol-3- yl)cyclohexyl)(2- cyclopropylethyl)amino)-1- oxoisoindolin-2-yl)piperidine-2,6- dione |
| 574 | 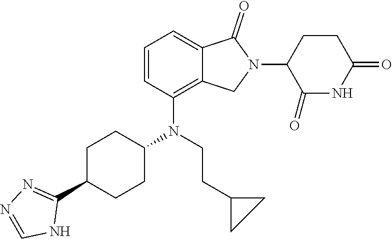 [SMILES: O=C1CCC(N2Cc3c(cccc3N(CCC3CC3)[C@H]3CC[C@H](c4nnc[nH]4)CC3)C2=O)C(=O)N1] | 3-(4-(((1r,4r)-4-(4H-1,2,4-triazol-3- yl)cyclohexyl)(2- cyclopropylethyl)amino)-1- oxoisoindolin-2-yl)piperidine-2,6- dione |
| 575 | 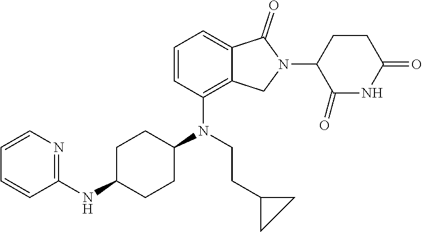 [SMILES: O=C1CCC(N2Cc3c(cccc3N(CCC3CC3)[C@H]3CC[C@@H](Nc4ccccn4)CC3)C2=O)C(=O)N1] | 3-(4-((2-cyclopropylethyl)((1s,4s)-4- (pyridin-2- ylamino)cyclohexyl)amino)-1- oxoisoindolin-2-yl)piperidine-2,6- dione |
| 576 | 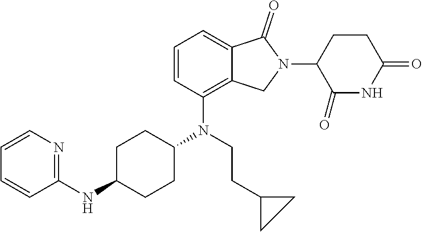 [SMILES: O=C1CCC(N2Cc3c(cccc3N(CCC3CC3)[C@H]3CC[C@H](Nc4ccccn4)CC3)C2=O)C(=O)N1] | 3-(4-((2-cyclopropylethyl)((lr,4r)-4- (pyridin-2- ylamino)cyclohexyl)amino)-1- oxoisoindolin-2-yl)piperidine-2,6- dione |
| 577 | 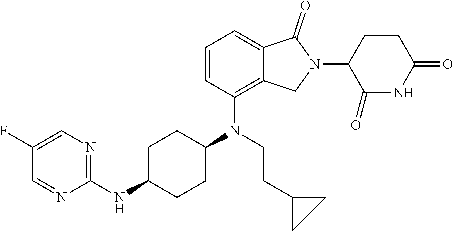 [SMILES: O=C1CCC(N2Cc3c(cccc3N(CCC3CC3)[C@H]3CC[C@@H](Nc4ncc(F)cn4)CC3)C2=O)C(=O)N1] | 3-(4-((2-cyclopropylethyl)((1s,4s)-4- ((5-fluoropyrimidin-2- yl)amino)cyclohexyl)amino)-1- oxoisoindolin-2-yl)piperidine-2,6- dione |
| 578 | 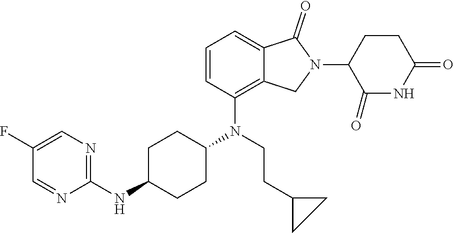 [SMILES: O=C1CCC(N2Cc3c(cccc3N(CCC3CC3)[C@H]3CC[C@H](Nc4ncc(F)cn4)CC3)C2=O)C(=O)N1] | 3-(4-((2-cyclopropylethyl)((lr,4r)-4- ((5-fluoropyrimidin-2- yl)amino)cyclohexyl)amino)-1- oxoisoindolin-2-yl)piperidine-2,6- dione |
| 580 | 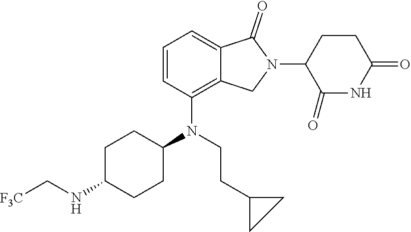 [SMILES: O=C1CCC(N2Cc3c(cccc3N(CCC3CC3)[C@H]3CC[C@H](NCC(F)(F)F)CC3)C2=O)C(=O)N1] | 3-(4-((2-cyclopropylethyl)((lr,4r)-4- ((2,2,2- trifluoroethyl)amino)cyclohexyl) amino)-1-oxoisoindolin-2- yl)piperidine-2,6-dione |
| 581 | 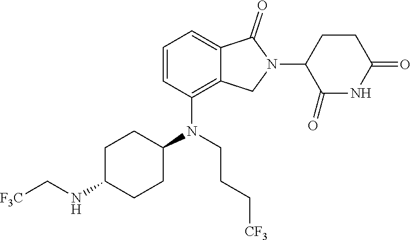 [SMILES: O=C1CCC(N2Cc3c(cccc3N(CCCC(F)(F)F)[C@H]3CC[C@H](NCC(F)(F)F)CC3)C2=O)C(=O)N1] | 3-(1-oxo-4-((4,4,4- trifluorobutyl)((1r,4r)-4-((2,2,2- trifluoroethyl)amino)cyclohexyl) amino)isoindolin-2- yl)piperidine-2,6-dione |
| 582 | 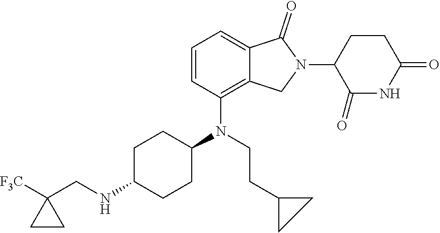 [SMILES: O=C1CCC(N2Cc3c(cccc3N(CCC3CC3)[C@H]3CC[C@H](NCC4(C(F)(F)F)CC4)CC3)C2=O)C(=O)N1] | 3-(4-((2-cyclopropylethyl)((lr,4r)-4- (((1- (trifluoromethyl)cyclopropyl)methyl) amino)cyclohexyl)amino)-1- oxoisoindolin-2-yl)piperidine-2,6- dione |
| 583 | 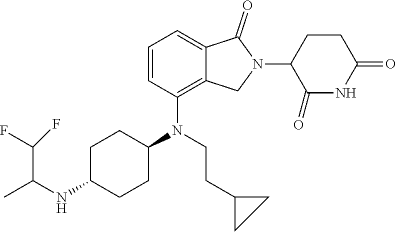 [SMILES: CC(N[C@H]1CC[C@H](N(CCC2CC2)c2cccc3c2CN(C2CCC(=O)NC2=O)C3=O)CC1)C(F)F] | 3-(4-((2-cyclopropylethyl)((lr,4r)-4- ((1,1-difluoropropan-2- yl)amino)cyclohexyl)amino)-1- oxoisoindolin-2-yl)piperidine-2,6- dione |
| 603 | 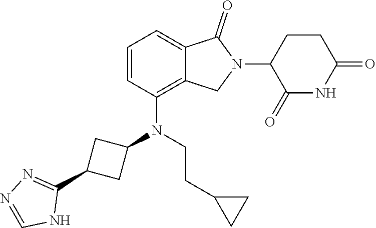 [SMILES: O=C1CCC(N2Cc3c(cccc3N(CCC3CC3)[C@H]3C[C@@H](c4nnc[nH]4)C3)C2=O)C(=O)N1] | 3-(4-(((1s,3s)-3-(4H-1,2,4-triazol-3- yl)cyclobutyl)(2- cyclopropylethyl)amino)-1- oxoisoindolin-2-yl)piperidine-2,6- dione |
| 604 | 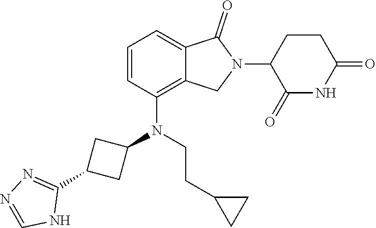 [SMILES: O=C1CCC(N2Cc3c(cccc3N(CCC3CC3)[C@H]3C[C@H](c4nnc[nH]4)C3)C2=O)C(=O)N1] | 3-(4-(((1r,3r)-3-(4H-1,2,4-triazol-3- yl)cyclobutyl)(2- cyclopropylethyl)amino)-1- oxoisoindolin-2-yl)piperidine-2,6- dione |
| 611 | 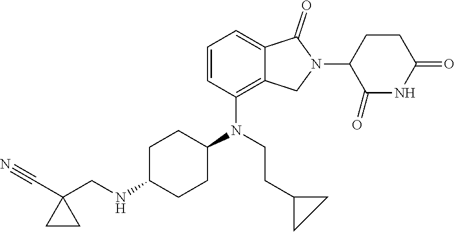 [SMILES: N#CC1(CN[C@H]2CC[C@H](N(CCC3CC3)c3cccc4c3CN(C3CCC(=O)NC3=O)C4=O)CC2)CC1] | 1-((((lr,4r)-4-((2-cyclopropylethyl)(2- (2,6-dioxopiperidin-3-yl)-1- oxoisoindolin-4- yl)amino)cyclohexyl)amino)methyl) cyclopropanecarbonitrile |
| 612 | 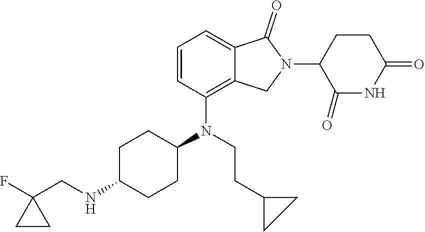 [SMILES: O=C1CCC(N2Cc3c(cccc3N(CCC3CC3)[C@H]3CC[C@H](NCC4(F)CC4)CC3)C2=O)C(=O)N1] | 3-(4-((2-cyclopropylethyl)((lr,4r)-4- (((1- fluorocyclopropyl)methyl)amino) cyclohexyl)amino)-1-oxoisoindolin-2- yl)piperidine-2,6-dione |
| 612A | 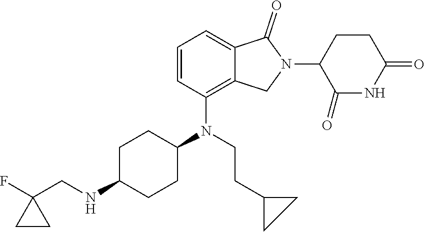 [SMILES: O=C1CCC(N2Cc3c(cccc3N(CCC3CC3)[C@H]3CC[C@@H](NCC4(F)CC4)CC3)C2=O)C(=O)N1] | 3-(4-((2-cyclopropylethyl)((1s,4s)-4- (((1- fluorocyclopropyl)methyl)amino) cyclohexyl)amino)-1-oxoisoindolin-2- yl)piperidine-2,6-dione |
| 613 | 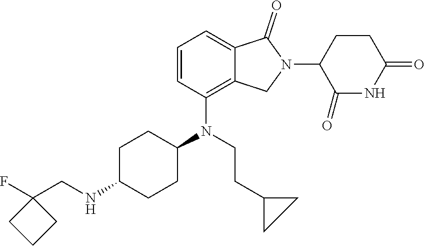 [SMILES: O=C1CCC(N2Cc3c(cccc3N(CCC3CC3)[C@H]3CC[C@H](NCC4(F)CCC4)CC3)C2=O)C(=O)N1] | 3-(4-((2-cyclopropylethyl)((lr,4r)-4- (((1- fluorocyclobutyl)methyl)amino) cyclohexyl)amino)-1-oxoisoindolin-2- yl)piperidine-2,6-dione |
| 613A | 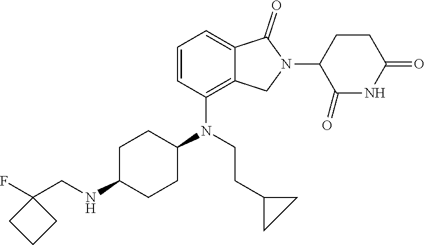 [SMILES: O=C1CCC(N2Cc3c(cccc3N(CCC3CC3)[C@H]3CC[C@@H](NCC4(F)CCC4)CC3)C2=O)C(=O)N1] | 3-(4-((2-cyclopropylethyl)((1s,4s)-4- (((1- fluorocyclobutyl)methyl)amino) cyclohexyl)amino)-1-oxoisoindolin-2- yl)piperidine-2,6-dione |
| 614 | 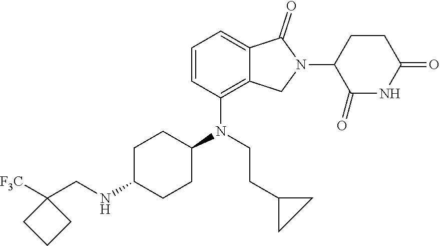 [SMILES: O=C1CCC(N2Cc3c(cccc3N(CCC3CC3)[C@H]3CC[C@H](NCC4(C(F)(F)F)CCC4)CC3)C2=O)C(=O)N1] | 3-(4-((2-cyclopropylethyl)((lr,4r)-4- (((1- (trifluoromethyl)cyclobutyl)methyl) amino)cyclohexyl)amino)-1- oxoisoindolin-2-yl)piperidine-2,6- dione |
| 614A | 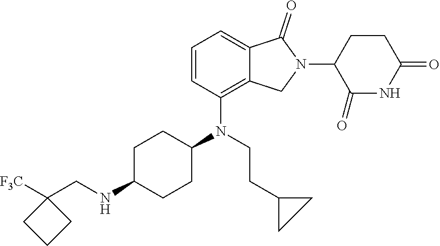 [SMILES: O=C1CCC(N2Cc3c(cccc3N(CCC3CC3)[C@H]3CC[C@@H](NCC4(C(F)(F)F)CCC4)CC3)C2=O)C(=O)N1] | 3-(4-((2-cyclopropylethyl)((1s,4s)-4- (((1- (trifluoromethyl)cyclobutyl)methyl) amino)cyclohexyl)amino)-1- oxoisoindolin-2-yl)piperidine-2,6- dione |
| 615 | 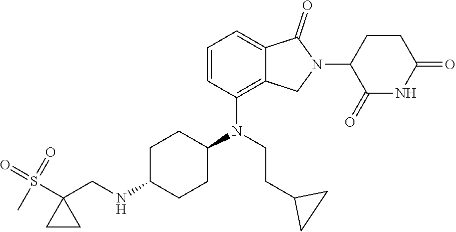 [SMILES: CS(=O)(=O)C1(CN[C@H]2CC[C@H](N(CCC3CC3)c3cccc4c3CN(C3CCC(=O)NC3=O)C4=O)CC2)CC1] | 3-(4-((2-cyclopropylethyl)((lr,4r)-4- (((1- (methylsulfonyl)cyclopropyl)methyl) amino)cyclohexyl)amino)-1- oxoisoindolin-2-yl)piperidine-2,6- dione |
| 615A | 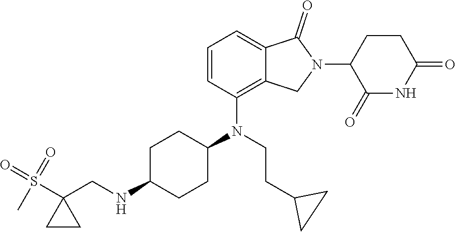 [SMILES: CS(=O)(=O)C1(CN[C@H]2CC[C@@H](N(CCC3CC3)c3cccc4c3CN(C3CCC(=O)NC3=O)C4=O)CC2)CC1] | 3-(4-((2-cyclopropylethyl)((1s,4s)-4- (((1- (methylsulfonyl)cyclopropyl)methyl) amino)cyclohexyl)amino)-1- oxoisoindolin-2-yl)piperidine-2,6- dione |
| 616 | 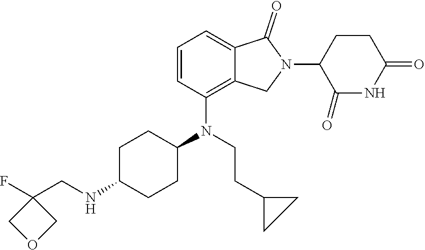 [SMILES: O=C1CCC(N2Cc3c(cccc3N(CCC3CC3)[C@H]3CC[C@H](NCC4(F)COC4)CC3)C2=O)C(=O)N1] | 3-(4-((2-cyclopropylethyl)((lr,4r)-4- (((3-fluorooxetan-3- yl)methyl)amino)cyclohexyl)amino)-1- oxoisoindolin-2-yl)piperidine-2,6- dione |
| 616A | 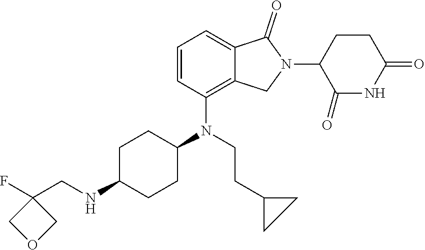 [SMILES: O=C1CCC(N2Cc3c(cccc3N(CCC3CC3)[C@H]3CC[C@@H](NCC4(F)COC4)CC3)C2=O)C(=O)N1] | 3-(4-((2-cyclopropylethyl)((1s,4s)-4- (((3-fluorooxetan-3- yl)methyl)amino)cyclohexyl)amino)-1- oxoisoindolin-2-yl)piperidine-2,6- dione |
| 619 | 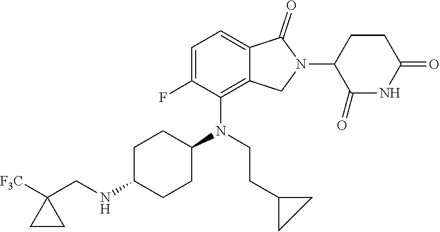 [SMILES: O=C1CCC(N2Cc3c(ccc(F)c3N(CCC3CC3)[C@H]3CC[C@H](NCC4(C(F)(F)F)CC4)CC3)C2=O)C(=O)N1] | 3-(4-((2-cyclopropylethyl)((lr,4r)-4- (((1- (trifluoromethyl)cyclopropyl)methyl) amino)cyclohexyl)amino)-5-fluoro-1- oxoisoindolin-2-yl)piperidine-2,6- dione |
| 627 | 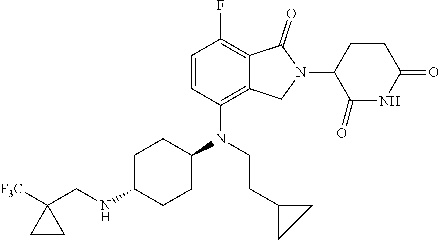 [SMILES: O=C1CCC(N2Cc3c(N(CCC4CC4)[C@H]4CC[C@H](NCC5(C(F)(F)F)CC5)CC4)ccc(F)c3C2=O)C(=O)N1] | 3-(4-((2-cyclopropylethyl)((1r,4r)-4- (((1- (trifluoromethyl)cyclopropyl)methyl) amino)cyclohexyl)amino)-7-fluoro-1- oxoisoindolin-2-yl)piperidine-2,6- dione |
| 627A | 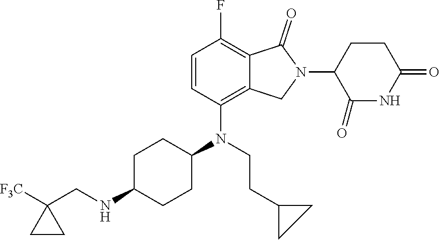 [SMILES: O=C1CCC(N2Cc3c(N(CCC4CC4)[C@H]4CC[C@@H](NCC5(C(F)(F)F)CC5)CC4)ccc(F)c3C2=O)C(=O)N1] | 3-(4-((2-cyclopropylethyl)((1s,4s)-4- (((1- (trifluoromethyl)cyclopropyl)methyl) amino)cyclohexyl)amino)-7-fluoro-1- oxoisoindolin-2-yl)piperidine-2,6- dione |
| 628 | 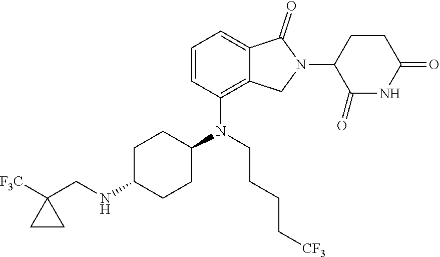 [SMILES: O=C1CCC(N2Cc3c(cccc3N(CCCCC(F)(F)F)[C@H]3CC[C@H](NCC4(C(F)(F)F)CC4)CC3)C2=O)C(=O)N1] | 3-(1-oxo-4-(((1r,4r)-4-(((1- (trifluoromethyl)cyclopropyl)methyl) amino)cyclohexyl)(5,5,5- trifluoropentyl)amino)isoindolin-2- yl)piperidine-2,6-dione |
| 628A | 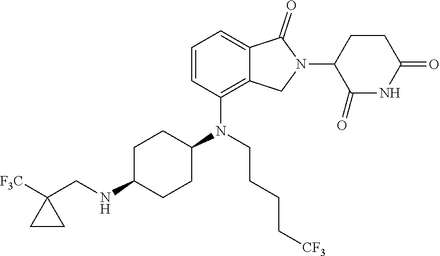 [SMILES: O=C1CCC(N2Cc3c(cccc3N(CCCCC(F)(F)F)[C@H]3CC[C@@H](NCC4(C(F)(F)F)CC4)CC3)C2=O)C(=O)N1] | 3-(1-oxo-4-(((1s,4s)-4-(((1- (trifluoromethyl)cyclopropyl)methyl)a mino)cyclohexyl)(5,5,5- trifluoropentyl)amino)isoindolin-2- yl)piperidine-2,6-dione |
| 629 | 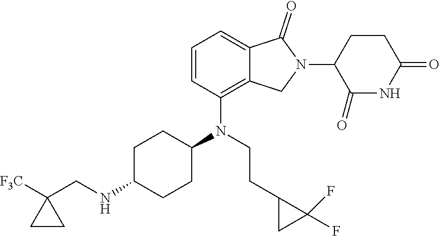 [SMILES: O=C1CCC(N2Cc3c(cccc3N(CCC3CC3(F)F)[C@H]3CC[C@H](NCC4(C(F)(F)F)CC4)CC3)C2=O)C(=O)N1] | 3-(4-((2-(2,2- difluorocyclopropyl)ethyl)((1r,4r)-4- (((1- (trifluoromethyl)cyclopropyl)methyl) amino)cyclohexyl)amino)-1- oxoisoindolin-2-yl)piperidine-2,6- dione |
| 629A | 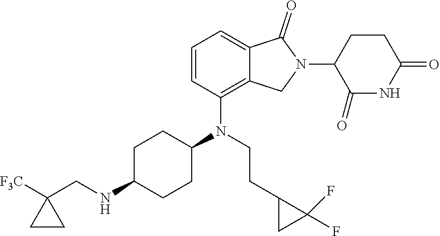 [SMILES: O=C1CCC(N2Cc3c(cccc3N(CCC3CC3(F)F)[C@H]3CC[C@@H](NCC4(C(F)(F)F)CC4)CC3)C2=O)C(=O)N1] | 3-(4-((2-(2,2- difluorocyclopropyl)ethyl)((1s,4s)-4- (((1- (trifluoromethyl)cyclopropyl)methyl) amino)cyclohexyl)amino)-1- oxoisoindolin-2-yl)piperidine-2,6- dione |
| 630 | 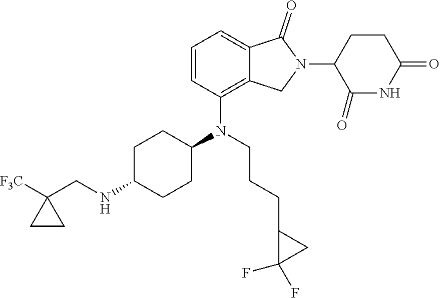 [SMILES: O=C1CCC(N2Cc3c(cccc3N(CCCC3CC3(F)F)[C@H]3CC[C@H](NCC4(C(F)(F)F)CC4)CC3)C2=O)C(=O)N1] | 3-(4-((3-(2,2- difluorocyclopropyl)propyl)((1r,4r)-4- (((1- (trifluoromethyl)cyclopropyl)methyl) amino)cyclohexyl)amino)-1- oxoisoindolin-2-yl)piperidine-2,6- dione |
| 630A | 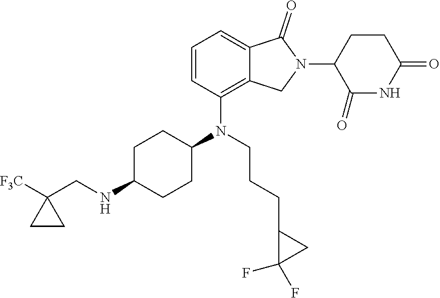 [SMILES: O=C1CCC(N2Cc3c(cccc3N(CCCC3CC3(F)F)[C@H]3CC[C@@H](NCC4(C(F)(F)F)CC4)CC3)C2=O)C(=O)N1] | 3-(4-((3-(2,2- difluorocyclopropyl)propyl)((1s,4s)-4- (((1- (trifluoromethyl)cyclopropyl)methyl)a mino)cyclohexyl)amino)-1- oxoisoindolin-2-yl)piperidine-2,6- dione |
| 634 | 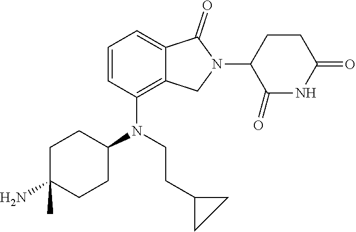 [SMILES: C[C@]1(N)CC[C@H](N(CCC2CC2)c2cccc3c2CN(C2CCC(=O)NC2=O)C3=O)CC1] | 3-(4-(((1r,4r)-4-amino-4- methylcyclohexyl)(2- cyclopropylethyl)amino)-1- oxoisoindolin-2-yl)piperidine-2,6- dione |
| 635 | 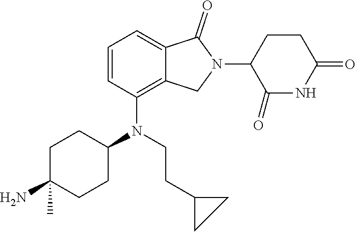 [SMILES: C[C@]1(N)CC[C@@H](N(CCC2CC2)c2cccc3c2CN(C2CCC(=O)NC2=O)C3=O)CC1] | 3-(4-(((1s,4s)-4-amino-4- methylcyclohexyl)(2- cyclopropylethyl)amino)-1- oxoisoindolin-2-yl)piperidine-2,6- dione |
| 636 | 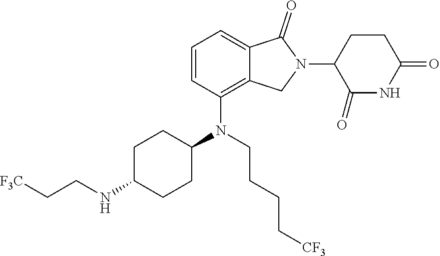 [SMILES: O=C1CCC(N2Cc3c(cccc3N(CCCCC(F)(F)F)[C@H]3CC[C@H](NCCC(F)(F)F)CC3)C2=O)C(=O)N1] | 3-(1-oxo-4-((5,5,5- trifluoropentyl)((lr,4r)-4-((3,3,3- trifluoropropyl)amino)cyclohexyl) amino)isoindolin-2- yl)piperidine-2,6-dione |
| 636A | 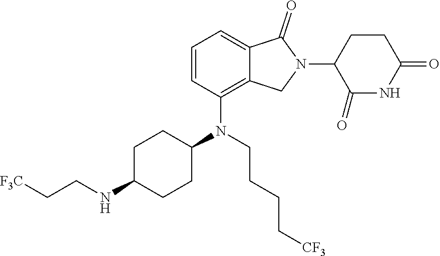 [SMILES: O=C1CCC(N2Cc3c(cccc3N(CCCCC(F)(F)F)[C@H]3CC[C@@H](NCCC(F)(F)F)CC3)C2=O)C(=O)N1] | 3-(1-oxo-4-((5,5,5- trifluoropentyl)((1s,4s)-4-((3,3,3- trifluoropropyl)amino)cyclohexyl) amino)isoindolin-2- yl)piperidine-2,6-dione |
| 637 | 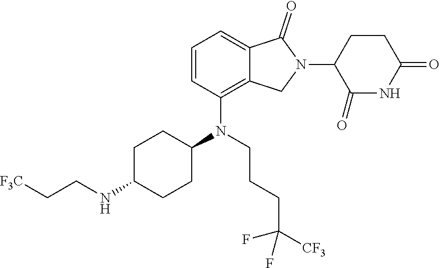 [SMILES: O=C1CCC(N2Cc3c(cccc3N(CCCC(F)(F)C(F)(F)F)[C@H]3CC[C@H](NCCC(F)(F)F)CC3)C2=O)C(=O)N1] | 3-(1-oxo-4-((4,4,5,5,5- pentafluoropentyl)((lr,4r)-4-((3,3,3- trifluoropropyl)amino)cyclohexyl) amino)isoindolin-2- yl)piperidine-2,6-dione |
| 637A | 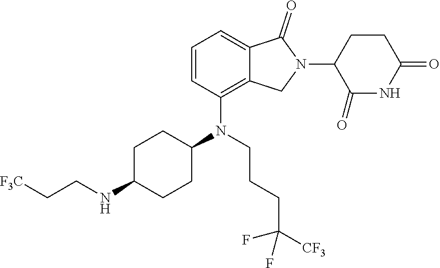 [SMILES: O=C1CCC(N2Cc3c(cccc3N(CCCC(F)(F)C(F)(F)F)[C@H]3CC[C@@H](NCCC(F)(F)F)CC3)C2=O)C(=O)N1] | 3-(1-oxo-4-((4,4,5,5,5- pentafluoropentyl)((1s,4s)-4-((3,3,3- trifluoropropyl)amino)cyclohexyl) amino)isoindolin-2- yl)piperidine-2,6-dione |
| 639 | 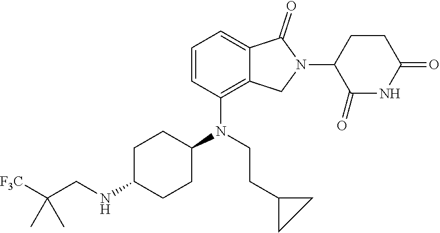 [SMILES: CC(C)(CN[C@H]1CC[C@H](N(CCC2CC2)c2cccc3c2CN(C2CCC(=O)NC2=O)C3=O)CC1)C(F)(F)F] | 3-(4-((2-cyclopropylethyl)((lr,4r)-4- ((3,3,3-trifluoro-2,2- dimethylpropyl)amino)cyclohexyl) amino)-1-oxoisoindolin-2- yl)piperidine- 2,6-dione |
| 639A | 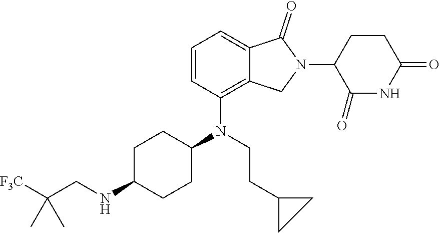 [SMILES: CC(C)(CN[C@H]1CC[C@@H](N(CCC2CC2)c2cccc3c2CN(C2CCC(=O)NC2=O)C3=O)CC1)C(F)(F)F] | 3-(4-((2-cyclopropylethyl)((1s,4s)-4- ((3,3,3-trifluoro-2,2- dimethylpropyl)amino)cyclohexyl) amino)-1-oxoisoindolin-2- yl)piperidine-2,6-dione |
| 642 | 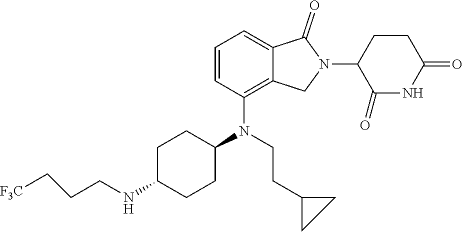 [SMILES: O=C1CCC(N2Cc3c(cccc3N(CCC3CC3)[C@H]3CC[C@H](NCCCC(F)(F)F)CC3)C2=O)C(=O)N1] | 3-(4-((2-cyclopropylethyl)((1r,4r)-4- ((4,4,4- trifluorobutyl)amino)cyclohexyl) amino)-1-oxoisoindolin-2- yl)piperidine-2,6-dione |
| 642A | 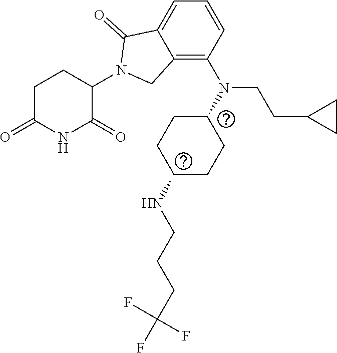 [SMILES: O=C1CCC(N2Cc3c(cccc3N(CCC3CC3)[C@H]3CC[C@@H](NCCCC(F)(F)F)CC3)C2=O)C(=O)N1] | 3-(4-((2-cyclopropylethyl)((1s,4s)-4- ((4,4,4- trifluorobutyl)amino)cyclohexyl) amino)-1-oxoisoindolin-2- yl)piperidine-2,6- dione |
| 643 | 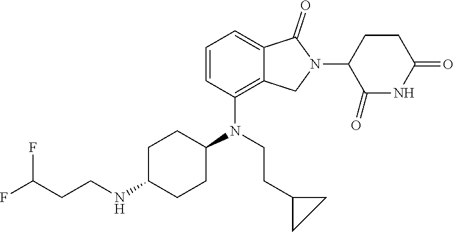 [SMILES: O=C1CCC(N2Cc3c(cccc3N(CCC3CC3)[C@H]3CC[C@H](NCCC(F)F)CC3)C2=O)C(=O)N1] | 3-(4-((2-cyclopropylethyl)((lr,4r)-4- ((3,3- difluoropropyl)amino)cyclohexyl) amino)-1-oxoisoindolin-2- yl)piperidine-2,6-dione |
| 643A | 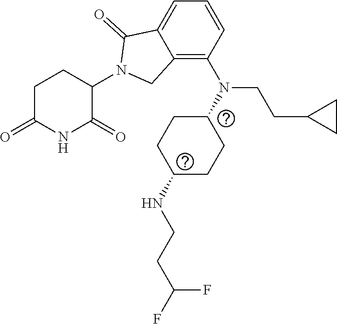 [SMILES: O=C1CCC(N2Cc3c(cccc3N(CCC3CC3)[C@H]3CC[C@@H](NCCC(F)F)CC3)C2=O)C(=O)N1] | 3-(4-((2-cyclopropylethyl)((1s,4s)-4- ((3,3- difluoropropyl)amino)cyclohexyl) amino)-1-oxoisoindolin-2- yl)piperidine-2,6-dione |
| 644 | 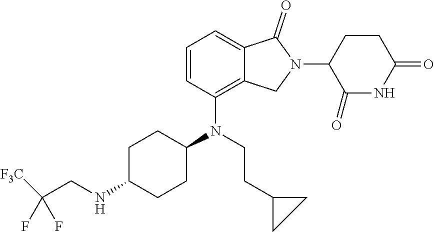 [SMILES: O=C1CCC(N2Cc3c(cccc3N(CCC3CC3)[C@H]3CC[C@H](NCC(F)(F)C(F)(F)F)CC3)C2=O)C(=O)N1] | 3-(4-((2-cyclopropylethyl)((lr,4r)-4- ((2,2,3,3,3- pentafluoropropyl)amino)cyclohexyl) amino)-1-oxoisoindolin-2- yl)piperidine-2,6-dione |
| 644A | 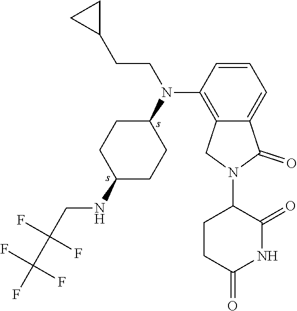 [SMILES: O=C1CCC(N2Cc3c(cccc3N(CCC3CC3)[C@H]3CC[C@@H](NCC(F)(F)C(F)(F)F)CC3)C2=O)C(=O)N1] | 3-(4-((2-cyclopropylethyl)((1s,4s)-4- ((2,2,3,3,3- pentafluoropropyl)amino)cyclohexyl) amino)-1-oxoisoindolin-2-yl)piperidine- 2,6-dione |
| 645 | 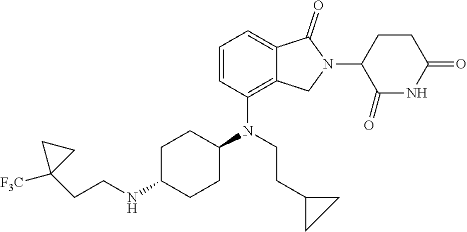 [SMILES: O=C1CCC(N2Cc3c(cccc3N(CCC3CC3)[C@H]3CC[C@H](NCCC4(C(F)(F)F)CC4)CC3)C2=O)C(=O)N1] | 3-(4-((2-cyclopropylethyl)((lr,4r)-4- ((2-(1- (trifluoromethyl)cyclopropyl)ethyl) amino)cyclohexyl)amino)-1- oxoisoindolin-2-yl)piperidine-2,6- dione |
| 645A | 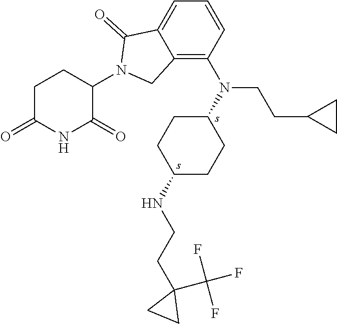 [SMILES: O=C1CCC(N2Cc3c(cccc3N(CCC3CC3)[C@H]3CC[C@@H](NCCC4(C(F)(F)F)CC4)CC3)C2=O)C(=O)N1] | 3-(4-((2-cyclopropylethyl)((1s,4s)-4- ((2-(1- (trifluoromethyl)cyclopropyl)ethyl) amino)cyclohexyl)amino)-1- oxoisoindolin-2-yl)piperidine-2,6- dione |
| 646 | 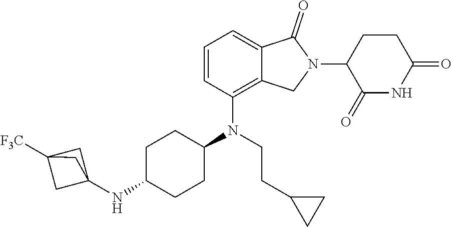 [SMILES: O=C1CCC(N2Cc3c(cccc3N(CCC3CC3)[C@H]3CC[C@H](NC45CC(C(F)(F)F)(C4)C5)CC3)C2=O)C(=O)N1] | 3-(4-((2-cyclopropylethyl)((1r,4r)-4- ((3- (trifluoromethyl)bicyclo[1.1.1]pentan- 1-yl)amino)cyclohexyl)amino)-1- oxoisoindolin-2-yl)piperidine-2,6- dione |
| 646A | 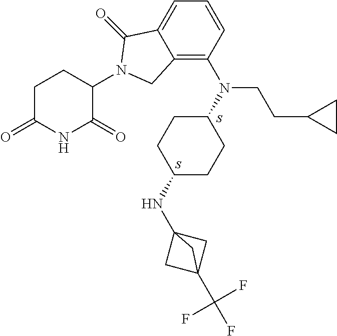 | 3-(4-((2-cyclopropylethyl)((1s,4s)-4- ((3- (trifluoromethyl)bicyclo[1.1.1]pentan- 1-yl)amino)cyclohexyl)amino)-1- oxoisoindolin-2-yl)piperidine-2,6- dione |
| 647 | 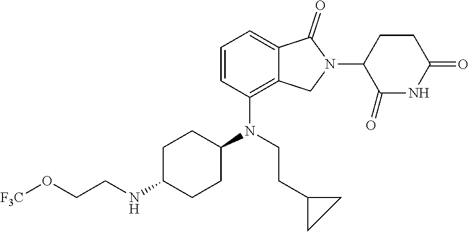 [SMILES: O=C1CCC(N2Cc3c(cccc3N(CCC3CC3)[C@H]3CC[C@H](NCCOC(F)(F)F)CC3)C2=O)C(=O)N1] | 3-(4-((2-cyclopropylethyl)((lr,4r)-4- ((2- (trifluoromethoxy)ethyl)amino) cyclohexyl)amino)-1- oxoisoindolin-2- yl)piperidine-2,6-dione |
| 647A | 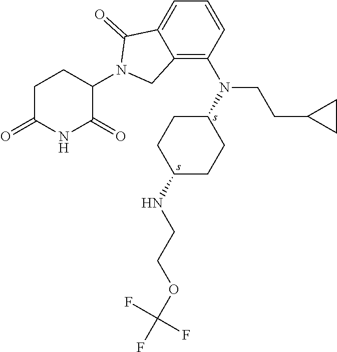 [SMILES: O=C1CCC(N2Cc3c(cccc3N(CCC3CC3)[C@H]3CC[C@@H](NCCOC(F)(F)F)CC3)C2=O)C(=O)N1] | 3-(4-((2-cyclopropylethyl)((1s,4s)-4- ((2- (trifluoromethoxy)ethyl)amino) cyclohexyl)amino)-1- oxoisoindolin-2- yl)piperidine-2,6-dione |
| 648 | 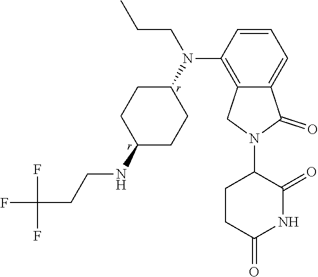 [SMILES: CCCN(c1cccc2c1CN(C1CCC(=O)NC1=O)C2=O)[C@H]1CC[C@H](NCCC(F)(F)F)CC1] | 3-(1-oxo-4-(propyl((lr,4r)-4-((3,3,3- trifluoropropyl)amino)cyclohexyl) amino)isoindolin-2- yl)piperidine-2,6-dione |
| 648A | 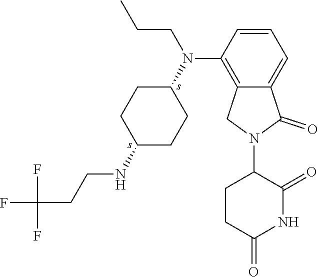 [SMILES: CCCN(c1cccc2c1CN(C1CCC(=O)NC1=O)C2=O)[C@H]1CC[C@@H](NCCC(F)(F)F)CC1] | 3-(1-oxo-4-(propyl((1s,4s)-4-((3,3,3- trifluoropropyl)amino)cyclohexyl) amino)isoindolin-2- yl)piperidine-2,6-dione |
| 649 | 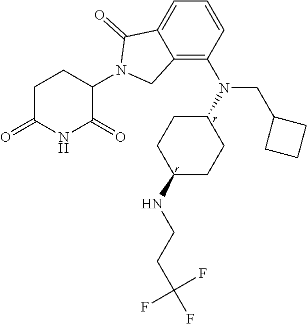 [SMILES: O=C1CCC(N2Cc3c(cccc3N(CC3CCC3)[C@H]3CC[C@H](NCCC(F)(F)F)CC3)C2=O)C(=O)N1] | 3-(4-((cyclobutylmethyl)((lr,4r)-4- ((3,3,3- trifluoropropyl)amino)cyclohexyl) amino)-1-oxoisoindolin-2- yl)piperidine-2,6-dione |
| 649A | 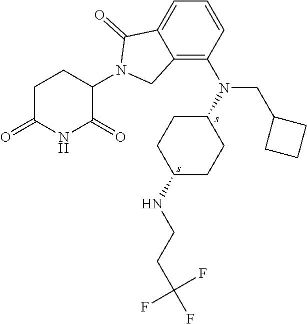 [SMILES: O=C1CCC(N2Cc3c(cccc3N(CC3CCC3)[C@H]3CC[C@@H](NCCC(F)(F)F)CC3)C2=O)C(=O)N1] | 3-(4-((cyclobutylmethyl)((1s,4s)-4- ((3,3,3- trifluoropropyl)amino)cyclohexyl) amino)-1-oxoisoindolin-2- yl)piperidine-2,6-dione |
| 650 | 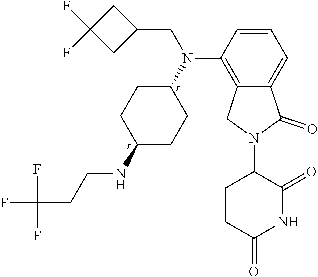 [SMILES: O=C1CCC(N2Cc3c(cccc3N(CC3CC(F)(F)C3)[C@H]3CC[C@H](NCCC(F)(F)F)CC3)C2=O)C(=O)N1] | 3-(4-(((3,3- difluorocyclobutyl)methyl)((lr,4r)-4- ((3,3,3- trifluoropropyl)amino)cyclohexyl) amino)-1-oxoisoindolin-2- yl)piperidine-2,6- dione |
| 650A | 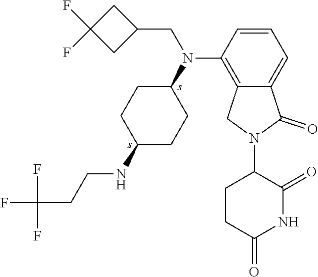 [SMILES: O=C1CCC(N2Cc3c(cccc3N(CC3CC(F)(F)C3)[C@H]3CC[C@@H](NCCC(F)(F)F)CC3)C2=O)C(=O)N1] | 3-(4-(((3,3- difluorocyclobutyl)methyl)((1s,4s)-4- ((3,3,3- trifluoropropyl)amino)cyclohexyl) amino)-1-oxoisoindolin-2- yl)piperidine-2,6- dione |
| 651 | 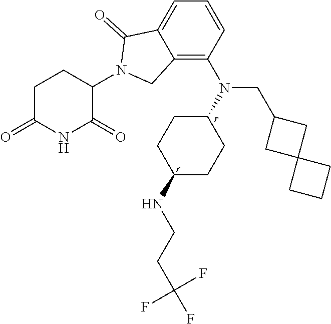 [SMILES: O=C1CCC(N2Cc3c(cccc3N(CC3CC4(CCC4)C3)[C@H]3CC[C@H](NCCC(F)(F)F)CC3)C2=O)C(=O)N1] | 3-(1-oxo-4-((spiro[3.3]heptan-2- ylmethyl)((lr,4r)-4-((3,3,3- trifluoropropyl)amino)cyclohexyl) amino)isoindolin-2- yl)piperidine-2,6-dione |
| 651A | 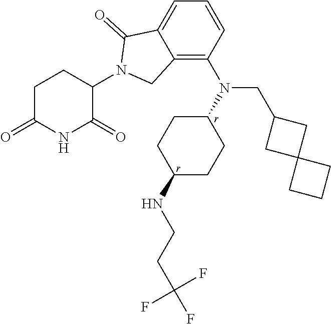 [SMILES: O=C1CCC(N2Cc3c(cccc3N(CC3CC4(CCC4)C3)[C@H]3CC[C@H](NCCC(F)(F)F)CC3)C2=O)C(=O)N1] | 3-(1-oxo-4-((spiro[3.3]heptan-2- ylmethyl)((1s,4s)-4-((3,3,3- trifluoropropyl)amino)cyclohexyl) amino)isoindolin-2- yl)piperidine-2,6-dione |
| 652 | 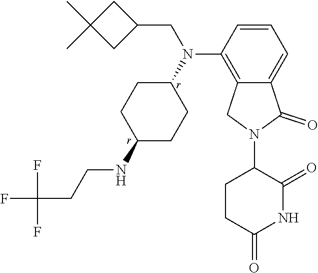 [SMILES: CC1(C)CC(CN(c2cccc3c2CN(C2CCC(=O)NC2=O)C3=O)[C@H]2CC[C@H](NCCC(F)(F)F)CC2)C1] | 3-(4-(((3,3- dimethylcyclobutyl)methyl)((1r,4r)-4- ((3,3,3- trifluoropropyl)amino)cyclohexyl) amino)-1-oxoisoindolin-2- yl)piperidine-2,6-dione |
| 652A | 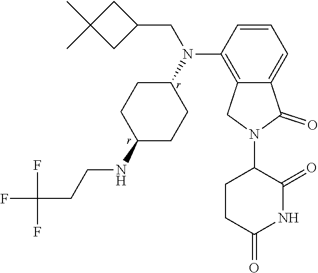 [SMILES: CC1(C)CC(CN(c2cccc3c2CN(C2CCC(=O)NC2=O)C3=O)[C@H]2CC[C@H](NCCC(F)(F)F)CC2)C1] | 3-(4-(((3,3- dimethylcyclobutyl)methyl)((1s,4s)-4- ((3,3,3- trifluoropropyl)amino)cyclohexyl) amino)-1-oxoisoindolin-2- yl)piperidine-2,6-dione |
| 653 | 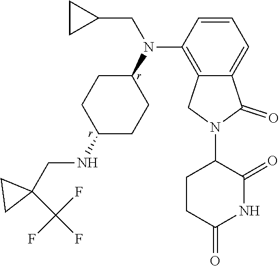 [SMILES: O=C1CCC(N2Cc3c(cccc3N(CC3CC3)[C@H]3CC[C@H](NCC4(C(F)(F)F)CC4)CC3)C2=O)C(=O)N1] | 3-(4-((cyclopropylmethyl)((1r,4r)-4- (((1- (trifluoromethyl)cyclopropyl)methyl) amino)cyclohexyl)amino)-1- oxoisoindolin-2-yl)piperidine-2,6- dione |
| 653A | 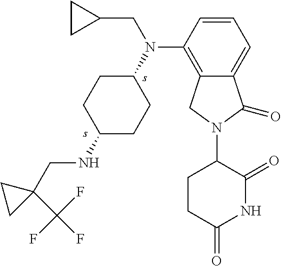 | 3-(4-((cyclopropylmethyl)((1s,4s)-4- (((1- (trifluoromethyl)cyclopropyl)methyl) amino)cyclohexyl)amino)-1- oxoisoindolin-2-yl)piperidine-2,6- dione |
| 654 | 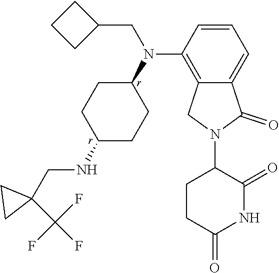 [SMILES: O=C1CCC(N2Cc3c(cccc3N(CC3CCC3)[C@H]3CC[C@H](NCC4(C(F)(F)F)CC4)CC3)C2=O)C(=O)N1] | 3-(4-((cyclobutylmethyl)((lr,4r)-4- (((1- (trifluoromethyl)cyclopropyl)methyl) amino)cyclohexyl)amino)-1- oxoisoindolin-2-yl)piperidine-2,6- dione |
| 654A | 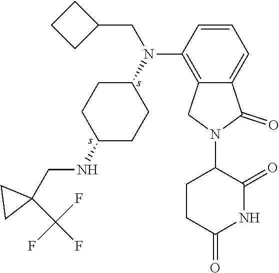 [SMILES: O=C1CCC(N2Cc3c(cccc3N(CC3CCC3)[C@H]3CC[C@@H](NCC4(C(F)(F)F)CC4)CC3)C2=O)C(=O)N1] | 3-(4-((cyclobutylmethyl)((1s,4s)-4- (((1- (trifluoromethyl)cyclopropyl)methyl) amino)cyclohexyl)amino)-1- oxoisoindolin-2-yl)piperidine-2,6- dione |
| 655 | 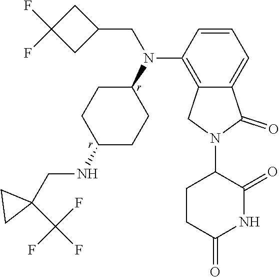 [SMILES: O=C1CCC(N2Cc3c(cccc3N(CC3CC(F)(F)C3)[C@H]3CC[C@H](NCC4(C(F)(F)F)CC4)CC3)C2=O)C(=O)N1] | 3-(4-(((3,3- difluorocyclobutyl)methyl)((lr,4r)-4- (((1- (trifluoromethyl)cyclopropyl)methyl) amino)cyclohexyl)amino)-1- oxoisoindolin-2-yl)piperidine-2,6- dione |
| 655A | 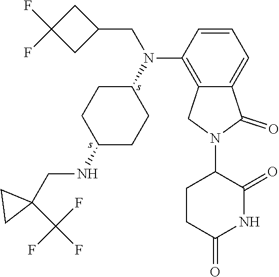 [SMILES: O=C1CCC(N2Cc3c(cccc3N(CC3CC(F)(F)C3)[C@H]3CC[C@@H](NCC4(C(F)(F)F)CC4)CC3)C2=O)C(=O)N1] | 3-(4-(((3,3- difluorocyclobutyl)methyl)((1s,4s)-4- (((1- (trifluoromethyl)cyclopropyl)methyl) amino)cyclohexyl)amino)-1- oxoisoindolin-2-yl)piperidine-2,6- dione |
| 656 | 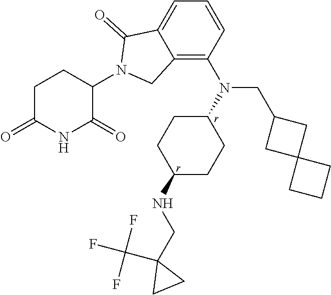 [SMILES: O=C1CCC(N2Cc3c(cccc3N(CC3CC4(CCC4)C3)[C@H]3CC[C@H](NCC4(C(F)(F)F)CC4)CC3)C2=O)C(=O)N1] | 3-(1-oxo-4-((spiro[3.3]heptan-2- ylmethyl)((1r,4r)-4-(((1- (trifluoromethyl)cyclopropyl)methyl) amino)cyclohexyl)amino)isoindolin-2- yl)piperidine-2,6-dione |
| 656A | 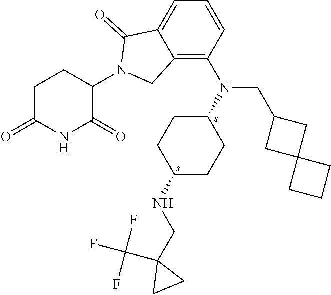 [SMILES: O=C1CCC(N2Cc3c(cccc3N(CC3CC4(CCC4)C3)[C@H]3CC[C@@H](NCC4(C(F)(F)F)CC4)CC3)C2=O)C(=O)N1] | 3-(1-oxo-4-((spiro[3.3]heptan-2- ylmethyl)((1s,4s)-4-(((1- (trifluoromethyl)cyclopropyl)methyl) amino)cyclohexyl)amino)isoindolin-2- yl)piperidine-2,6-dione |
| 657 | 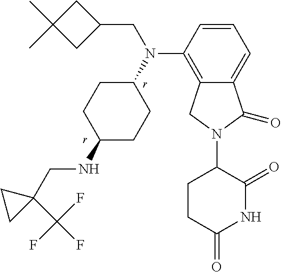 | 3-(4-(((3,3- dimethylcyclobutyl)methyl)((1r,4r)-4- (((1- (trifluoromethyl)cyclopropyl)methyl) amino)cyclohexyl)amino)-1- oxoisoindolin-2-yl)piperidine-2,6- dione |
| 657A | 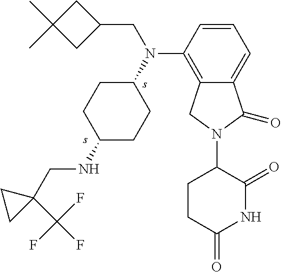 | 3-(4-(((3,3- dimethylcyclobutyl)methyl)((1s,4s)-4- (((1- (trifluoromethyl)cyclopropyl)methyl) amino)cyclohexyl)amino)-1- oxoisoindolin-2-yl)piperidine-2,6- dione |
| 658 | 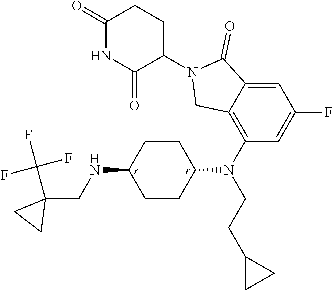 [SMILES: O=C1CCC(N2Cc3c(cc(F)cc3N(CCC3CC3)[C@H]3CC[C@H](NCC4(C(F)(F)F)CC4)CC3)C2=O)C(=O)N1] | 3-(4-((2-cyclopropylethyl)((lr,4r)-4- (((1- (trifluoromethyl)cyclopropyl)methyl) amino)cyclohexyl)amino)-6-fluoro-1- oxoisoindolin-2-yl)piperidine-2,6- dione |
| 658A | 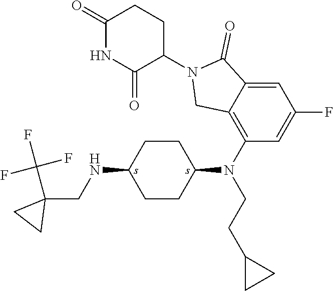 [SMILES: O=C1CCC(N2Cc3c(cc(F)cc3N(CCC3CC3)[C@H]3CC[C@@H](NCC4(C(F)(F)F)CC4)CC3)C2=O)C(=O)N1] | 3-(4-((2-cyclopropylethyl)((1s,4s)-4- (((1- (trifluoromethyl)cyclopropyl)methyl) amino)cyclohexyl)amino)-6-fluoro-1- oxoisoindolin-2-yl)piperidine-2,6- dione |
| 659 | 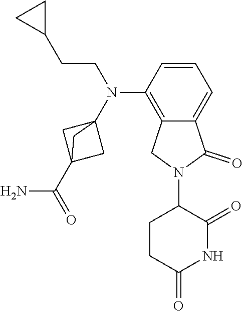 [SMILES: NC(=O)C12CC(N(CCC3CC3)c3cccc4c3CN(C3CCC(=O)NC3=O)C4=O)(C1)C2] | 3-((2-cyclopropylethyl)(2-(2,6- dioxopiperidin-3-yl)-1-oxoisoindolin- 4-yl)amino)bicyclo[1.1.1]pentane-1- carboxamide |
| 660 | 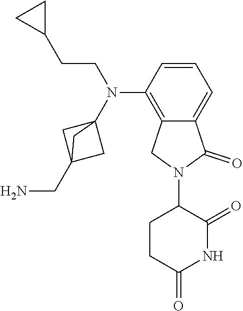 [SMILES: NCC12CC(N(CCC3CC3)c3cccc4c3CN(C3CCC(=O)NC3=O)C4=O)(C1)C2] | 3-(4-((3- (aminomethyl)bicyclo[1.1.1]pentan-1- yl)(2-cyclopropylethyl)amino)-1- oxoisoindolin-2-yl)piperidine-2,6- dione |
| 662 | 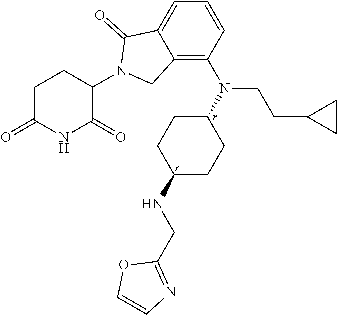 [SMILES: O=C1CCC(N2Cc3c(cccc3N(CCC3CC3)[C@H]3CC[C@H](NCc4ncco4)CC3)C2=O)C(=O)N1] | 3-(4-((2-cyclopropylethyl)((lr,4r)-4- ((oxazol-2- ylmethyl)amino)cyclohexyl)amino)-1- oxoisoindolin-2-yl)piperidine-2,6- dione |
| 663 | 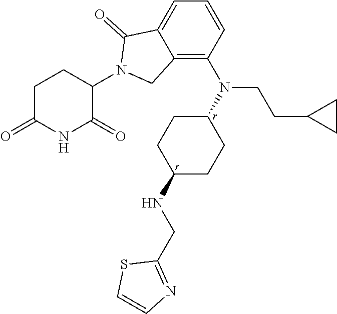 [SMILES: O=C1CCC(N2Cc3c(cccc3N(CCC3CC3)[C@H]3CC[C@H](NCc4nccs4)CC3)C2=O)C(=O)N1] | 3-(4-((2-cyclopropylethyl)((lr,4r)-4- ((thiazol-2- ylmethyl)amino)cyclohexyl)amino)-1- oxoisoindolin-2-yl)piperidine-2,6- dione |
| 664 | 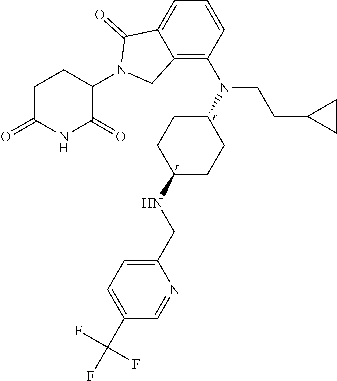 [SMILES: O=C1CCC(N2Cc3c(cccc3N(CCC3CC3)[C@H]3CC[C@H](NCc4ccc(C(F)(F)F)cn4)CC3)C2=O)C(=O)N1] | 3-(4-((2-cyclopropylethyl)((lr,4r)-4- (((5-(trifluoromethyl)pyridin-2- yl)methyl)amino)cyclohexyl)amino)-1- oxoisoindolin-2-yl)piperidine-2,6- dione |
| 665 | 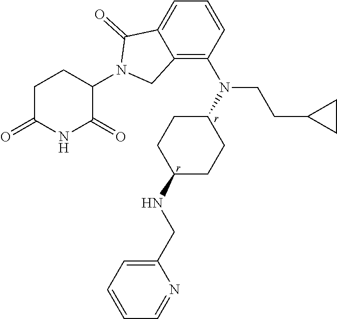 [SMILES: O=C1CCC(N2Cc3c(cccc3N(CCC3CC3)[C@H]3CC[C@H](NCc4ccccn4)CC3)C2=O)C(=O)N1] | 3-(4-((2-cyclopropylethyl)((lr,4r)-4- ((pyridin-2- ylmethyl)amino)cyclohexyl)amino)-1- oxoisoindolin-2-yl)piperidine-2,6- dione |
| 666 | 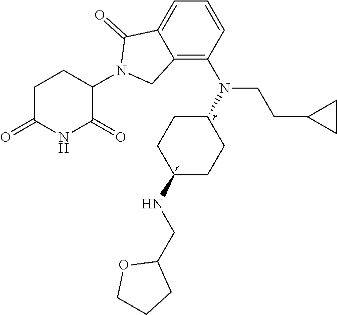 [SMILES: O=C1CCC(N2Cc3c(cccc3N(CCC3CC3)[C@H]3CC[C@H](NCC4CCCO4)CC3)C2=O)C(=O)N1] | 3-(4-((2-cyclopropylethyl)((lr,4r)-4- (((tetrahydrofuran-2- yl)methyl)amino)cyclohexyl)amino)-1- oxoisoindolin-2-yl)piperidine-2,6- dione |
| 668 | 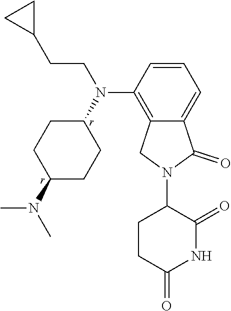 [SMILES: CN(C)[C@H]1CC[C@H](N(CCC2CC2)c2cccc3c2CN(C2CCC(=O)NC2=O)C3=O)CC1] | 3-(4-((2-cyclopropylethyl)((lr,4r)-4- (dimethylamino)cyclohexyl)amino)-1- oxoisoindolin-2-yl)piperidine-2,6- dione |
| 669 | 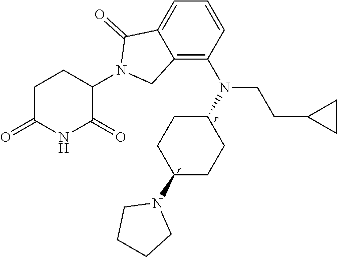 [SMILES: O=C1CCC(N2Cc3c(cccc3N(CCC3CC3)[C@H]3CC[C@H](N4CCCC4)CC3)C2=O)C(=O)N1] | 3-(4-((2-cyclopropylethyl)((lr,4r)-4- (pyrrolidin-1-yl)cyclohexyl)amino)-1- oxoisoindolin-2-yl)piperidine-2,6- dione |
| 669A | 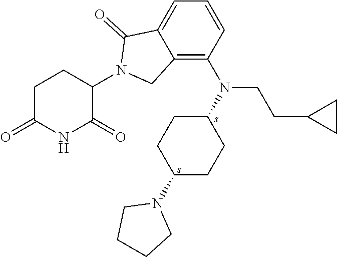 [SMILES: O=C1CCC(N2Cc3c(cccc3N(CCC3CC3)[C@H]3CC[C@@H](N4CCCC4)CC3)C2=O)C(=O)N1] | 3-(4-((2-cyclopropylethyl)((1s,4s)-4- (pyrrolidin-1-yl)cyclohexyl)amino)-1- oxoisoindolin-2-yl)piperidine-2,6- dione |
| 670 | 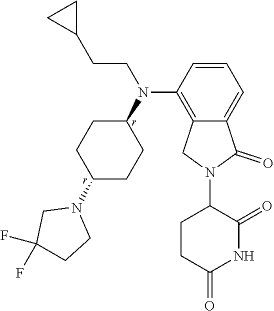 [SMILES: O=C1CCC(N2Cc3c(cccc3N(CCC3CC3)[C@H]3CC[C@H](N4CCC(F)(F)C4)CC3)C2=O)C(=O)N1] | 3-(4-((2-cyclopropylethyl)((1r,4r)-4- (3,3-difluoropyrrolidin-1- yl)cyclohexyl)amino)-1-oxoisoindolin- 2-yl)piperidine-2,6-dione |
| 670A | 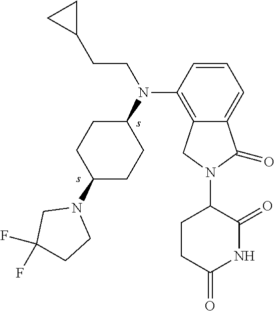 | 3-(4-((2-cyclopropylethyl)((1s,4s)-4- (3,3-difluoropyrrolidin-1- yl)cyclohexyl)amino)-1-oxoisoindolin- 2-yl)piperidine-2,6-dione |
| 671 | 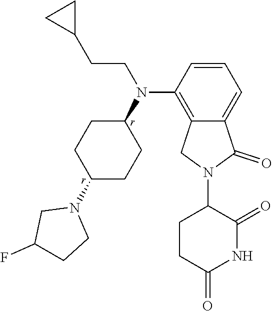 [SMILES: O=C1CCC(N2Cc3c(cccc3N(CCC3CC3)[C@H]3CC[C@H](N4CCC(F)C4)CC3)C2=O)C(=O)N1] | 3-(4-((2-cyclopropylethyl)((lr,4r)-4-(3- fluoropyrrolidin-1- yl)cyclohexyl)amino)-1-oxoisoindolin- 2-yl)piperidine-2,6-dione |
| 671A | 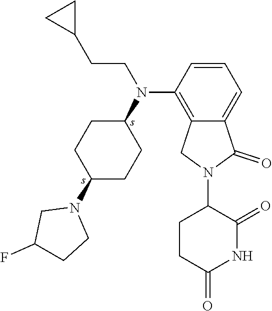 [SMILES: O=C1CCC(N2Cc3c(cccc3N(CCC3CC3)[C@H]3CC[C@@H](N4CCC(F)C4)CC3)C2=O)C(=O)N1] | 3-(4-((2-cyclopropylethyl)((1s,4s)-4- (3-fluoropyrrolidin-1- yl)cyclohexyl)amino)-1-oxoisoindolin- 2-yl)piperidine-2,6-dione |
| 672 | 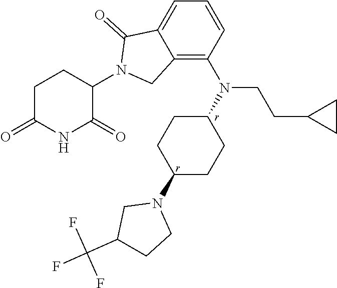 [SMILES: O=C1CCC(N2Cc3c(cccc3N(CCC3CC3)[C@H]3CC[C@H](N4CCC(C(F)(F)F)C4)CC3)C2=O)C(=O)N1] | 3-(4-((2-cyclopropylethyl)((1r,4r)-4-(3- (trifluoromethyl)pyrrolidin-1- yl)cyclohexyl)amino)-1-oxoisoindolin- 2-yl)piperidine-2,6-dione |
| 672A | 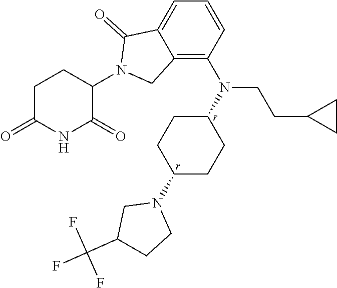 [SMILES: O=C1CCC(N2Cc3c(cccc3N(CCC3CC3)[C@H]3CC[C@@H](N4CCC(C(F)(F)F)C4)CC3)C2=O)C(=O)N1] | 3-(4-((2-cyclopropylethyl)((1s,4s)-4- (3-(trifluoromethyl)pyrrolidin-1- yl)cyclohexyl)amino)-1-oxoisoindolin- 2-yl)piperidine-2,6-dione |
| 673 | 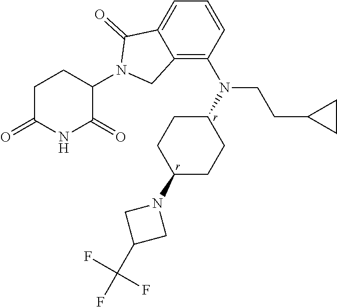 [SMILES: O=C1CCC(N2Cc3c(cccc3N(CCC3CC3)[C@H]3CC[C@H](N4CC(C(F)(F)F)C4)CC3)C2=O)C(=O)N1] | 3-(4-((2-cyclopropylethyl)((lr,4r)-4-(3- (trifluoromethyl)azetidin-1- yl)cyclohexyl)amino)-1-oxoisoindolin- 2-yl)piperidine-2,6-dione |
| 673A | 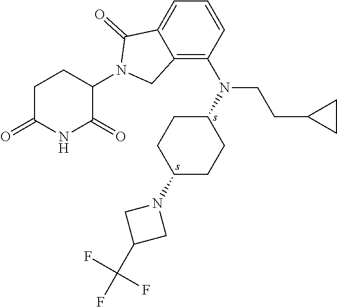 [SMILES: O=C1CCC(N2Cc3c(cccc3N(CCC3CC3)[C@H]3CC[C@@H](N4CC(C(F)(F)F)C4)CC3)C2=O)C(=O)N1] | 3-(4-((2-cyclopropylethyl)((1s,4s)-4- (3-(trifluoromethyl)azetidin-1- yl)cyclohexyl)amino)-1-oxoisoindolin- 2-yl)piperidine-2,6-dione |
| 674 | 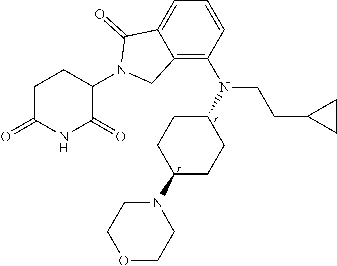 [SMILES: O=C1CCC(N2Cc3c(cccc3N(CCC3CC3)[C@H]3CC[C@H](N4CCOCC4)CC3)C2=O)C(=O)N1] | 3-(4-((2-cyclopropylethyl)((lr,4r)-4- morpholinocyclohexyl)amino)-1- oxoisoindolin-2-yl)piperidine-2,6- dione |
| 674A | 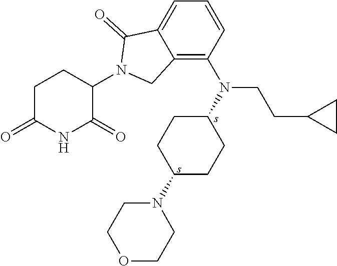 [SMILES: O=C1CCC(N2Cc3c(cccc3N(CCC3CC3)[C@H]3CC[C@@H](N4CCOCC4)CC3)C2=O)C(=O)N1] | 3-(4-((2-cyclopropylethyl)((1s,4s)-4- morpholinocyclohexyl)amino)-1- oxoisoindolin-2-yl)piperidine-2,6- dione |
| 675 | 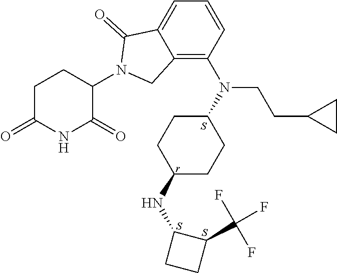 | 3-(4-((2-cyclopropylethyl)((1S,4r)-4- (((1S,2S)-2- (trifluoromethyl)cyclobutyl)amino) cyclohexyl)amino)-1-oxoisoindolin-2- yl)piperidine-2,6-dione |
| 675A | 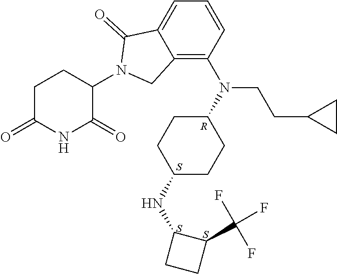 | 3-(4-((2-cyclopropylethyl)((1R,4s)-4- (((1S,2S)-2- (trifluoromethyl)cyclobutyl)amino) cyclohexyl)amino)-1- oxoisoindolin-2- yl)piperidine-2,6-dione |
| 678 | 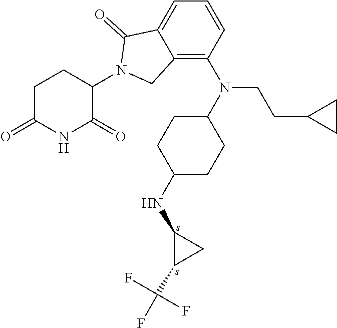 [SMILES: O=C1CCC(N2Cc3c(cccc3N(CCC3CC3)C3CCC(N[C@H]4C[C@@H]4C(F)(F)F)CC3)C2=O)C(=O)N1] | 3-(4-((2-cyclopropylethyl)(4-(((1S,2S)- 2-(trifluoromethyl)cyclopropyl)amino) cyclohexyl)amino)-1-oxoisoindolin-2- yl)piperidine-2,6-dione |
| 679 | 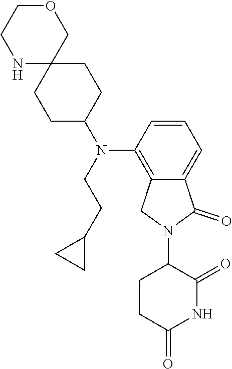 [SMILES: O=C1CCC(N2Cc3c(cccc3N(CCC3CC3)C3CCC4(CC3)COCCN4)C2=O)C(=O)N1] | 3-(4-((2-cyclopropylethyl)(4-oxa-1- azaspiro[5.5]undecan-9-yl)amino)-1- oxoisoindolin-2-yl)piperidine-2,6- dione |
| 680 | 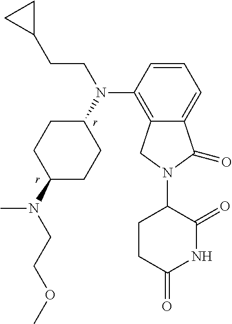 [SMILES: COCCN(C)[C@H]1CC[C@H](N(CCC2CC2)c2cccc3c2CN(C2CCC(=O)NC2=O)C3=O)CC1] | 3-(4-((2-cyclopropylethyl)((lr,4r)-4- ((2- methoxyethyl)(methyl)amino) cyclohexyl)amino)-1-oxoisoindolin-2- yl)piperidine-2,6-dione |
| 680A | 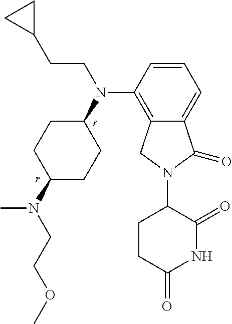 [SMILES: COCCN(C)[C@H]1CC[C@@H](N(CCC2CC2)c2cccc3c2CN(C2CCC(=O)NC2=O)C3=O)CC1] | 3-(4-((2-cyclopropylethyl)((1s,4s)-4- ((2- methoxyethyl)(methyl)amino) cyclohexyl)amino)-1- oxoisoindolin-2- yl)piperidine-2,6-dione |
| 681 | 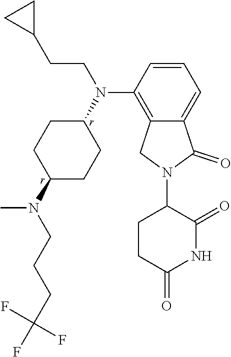 [SMILES: CN(CCCC(F)(F)F)[C@H]1CC[C@H](N(CCC2CC2)c2cccc3c2CN(C2CCC(=O)NC2=O)C3=O)CC1] | 3-(4-((2-cyclopropylethyl)((lr,4r)-4- (methyl(4,4,4- trifluorobutyl)amino)cyclohexyl) amino)-1-oxoisoindolin-2- yl)piperidine-2,6-dione |
| 681A | 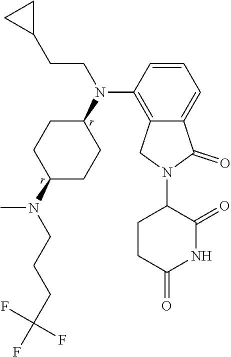 [SMILES: CN(CCCC(F)(F)F)[C@H]1CC[C@@H](N(CCC2CC2)c2cccc3c2CN(C2CCC(=O)NC2=O)C3=O)CC1] | 3-(4-((2-cyclopropylethyl)((1s,4s)-4- (methyl(4,4,4- trifluorobutyl)amino)cyclohexyl) amino)-1-oxoisoindolin-2- yl)piperidine-2,6-dione |
| 682 | 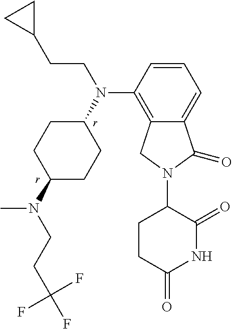 [SMILES: CN(CCC(F)(F)F)[C@H]1CC[C@H](N(CCC2CC2)c2cccc3c2CN(C2CCC(=O)NC2=O)C3=O)CC1] | 3-(4-((2-cyclopropylethyl)((lr,4r)-4- (methyl(3,3,3- trifluoropropyl)amino)cyclohexyl) amino)-1-oxoisoindolin-2- yl)piperidine-2,6-dione |
| 682A | 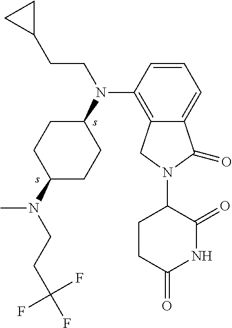 [SMILES: CN(CCC(F)(F)F)[C@H]1CC[C@@H](N(CCC2CC2)c2cccc3c2CN(C2CCC(=O)NC2=O)C3=O)CC1] | 3-(4-((2-cyclopropylethyl)((1s,4s)-4- (methyl(3,3,3- trifluoropropyl)amino)cyclohexyl) amino)-1-oxoisoindolin-2- yl)piperidine-2,6-dione |
| 683 | 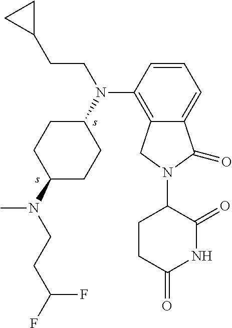 [SMILES: CN(CCC(F)F)[C@H]1CC[C@H](N(CCC2CC2)c2cccc3c2CN(C2CCC(=O)NC2=O)C3=O)CC1] | 3-(4-((2-cyclopropylethyl)((lr,4r)-4- ((3,3- difluoropropyl)(methyl)amino) cyclohexyl)amino)-1- oxoisoindolin-2- yl)piperidine-2,6-dione |
| 683A | 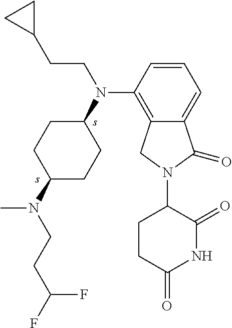 [SMILES: CN(CCC(F)F)[C@H]1CC[C@@H](N(CCC2CC2)c2cccc3c2CN(C2CCC(=O)NC2=O)C3=O)CC1] | 3-(4-((2-cyclopropylethyl)((1s,4s)-4- ((3,3- difluoropropyl)(methyl)amino) cyclohexyl)amino)-1- oxoisoindolin-2- yl)piperidine-2,6-dione |
| 703 | 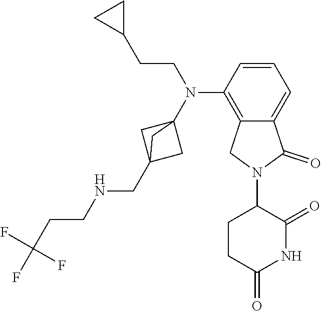 [SMILES: O=C1CCC(N2Cc3c(cccc3N(CCC3CC3)C34CC(CNCCC(F)(F)F)(C3)C4)C2=O)C(=O)N1] | 3-(4-((2-cyclopropylethyl)(3-(((3,3,3- trifluoropropyl)amino)methyl)bicyclo [1.1.1]pentan-1-yl)amino)-1- oxoisoindolin-2-yl)piperidine-2,6- dione |
| 708 | 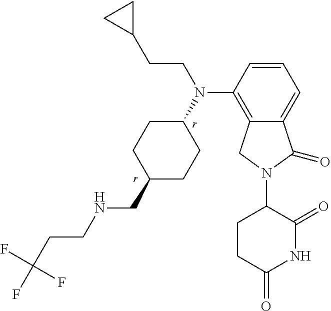 [SMILES: O=C1CCC(N2Cc3c(cccc3N(CCC3CC3)[C@H]3CC[C@H](CNCCC(F)(F)F)CC3)C2=O)C(=O)N1] | 3-(4-((2-cyclopropylethyl)((1r,4r)-4- (((3,3,3- trifluoropropyl)amino)methyl) cyclohexyl)amino)-1- oxoisoindolin-2- yl)piperidine-2,6-dione |
| 708A | 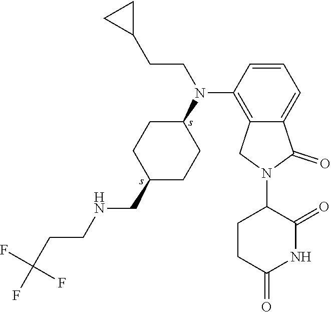 [SMILES: O=C1CCC(N2Cc3c(cccc3N(CCC3CC3)[C@H]3CC[C@@H](CNCCC(F)(F)F)CC3)C2=O)C(=O)N1] | 3-(4-((2-cyclopropylethyl)((1s,4s)-4- (((3,3,3- trifluoropropyl)amino)methyl) cyclohexyl)amino)-1- oxoisoindolin-2- yl)piperidine-2,6-dione |
| 711 | 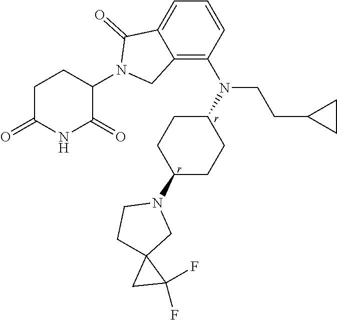 [SMILES: O=C1CCC(N2Cc3c(cccc3N(CCC3CC3)[C@H]3CC[C@H](N4CCC5(C4)CC5(F)F)CC3)C2=O)C(=O)N1] | 3-(4-((2-cyclopropylethyl)((lr,4r)-4- (1,1-difluoro-5-azaspiro[2.4]heptan-5- yl)cyclohexyl)amino)-1-oxoisoindolin- 2-yl)piperidine-2,6-dione |
| 711A | 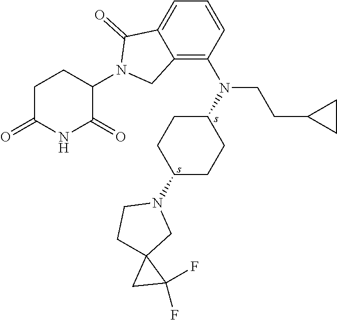 [SMILES: O=C1CCC(N2Cc3c(cccc3N(CCC3CC3)[C@H]3CC[C@@H](N4CCC5(C4)CC5(F)F)CC3)C2=O)C(=O)N1] | 3-(4-((2-cyclopropylethyl)((1s,4s)-4- (1,1-difluoro-5-azaspiro[2.4]heptan-5- yl)cyclohexyl)amino)-1-oxoisoindolin- 2-yl)piperidine-2,6-dione |
| 712 | 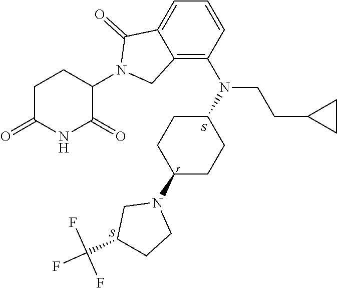 | 3-(4-((2-cyclopropylethyl)((1S,4r)-4- ((S)-3-(trifluoromethyl)pyrrolidin-1- yl)cyclohexyl)amino)-1-oxoisoindolin- 2-yl)piperidine-2,6-dione |
| 712A | 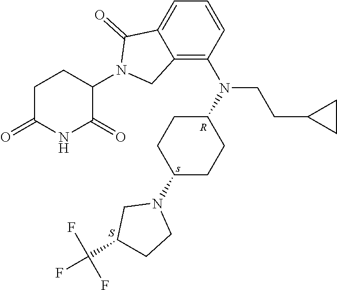 | 3-(4-((2-cyclopropylethyl)((1R,4s)-4- ((S)-3-(trifluoromethyl)pyrrolidin-1- yl)cyclohexyl)amino)-1-oxoisoindolin- 2-yl)piperidine-2,6-dione |
| 713 | 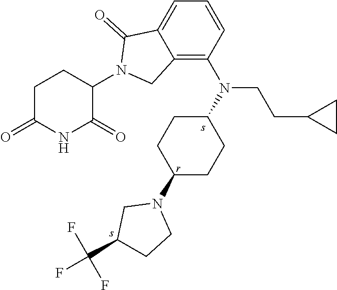 | 3-(4-((2-cyclopropylethyl)((1R,4r)-4- ((R)-3-(trifluoromethyl)pyrrolidin-1- yl)cyclohexyl)amino)-1-oxoisoindolin- 2-yl)piperidine-2,6-dione |
| 713A | 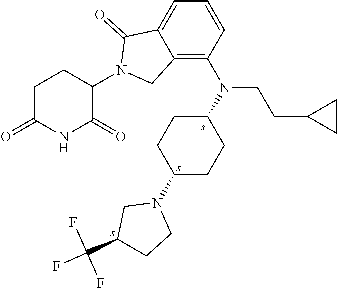 | 3-(4-((2-cyclopropylethyl)((1S,4s)-4- ((R)-3-(trifluoromethyl)pyrrolidin-1- yl)cyclohexyl)amino)-1-oxoisoindolin- 2-yl)piperidine-2,6-dione |
| 714 | 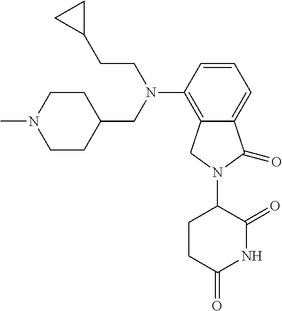 [SMILES: CN1CCC(CN(CCC2CC2)c2cccc3c2CN(C2CCC(=O)NC2=O)C3=O)CC1] | 3-(4-((2-cyclopropylethyl)((1- methylpiperidin-4-yl)methyl)amino)-1- oxoisoindolin-2-yl)piperidine-2,6- dione |
| 715 | 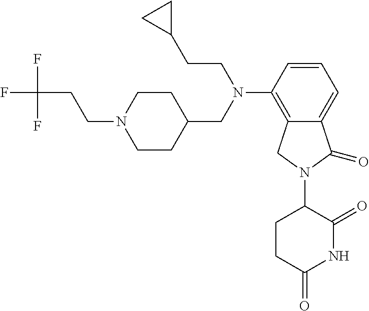 [SMILES: O=C1CCC(N2Cc3c(cccc3N(CCC3CC3)CC3CCN(CCC(F)(F)F)CC3)C2=O)C(=O)N1] | 3-(4-((2-cyclopropylethyl)((1-(3,3,3- trifluoropropyl)piperidin-4- yl)methyl)amino)-1-oxoisoindolin-2- yl)piperidine-2,6-dione |
| 716 | 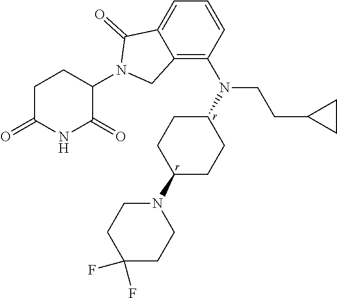 [SMILES: O=C1CCC(N2Cc3c(cccc3N(CCC3CC3)[C@H]3CC[C@H](N4CCC(F)(F)CC4)CC3)C2=O)C(=O)N1] | 3-(4-((2-cyclopropylethyl)((lr,4r)-4- (4,4-difluoropiperidin-1- yl)cyclohexyl)amino)-1-oxoisoindolin- 2-yl)piperidine-2,6-dione |
| 716A | 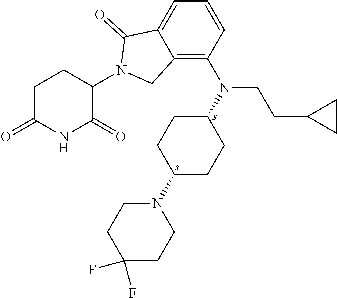 [SMILES: O=C1CCC(N2Cc3c(cccc3N(CCC3CC3)[C@H]3CC[C@@H](N4CCC(F)(F)CC4)CC3)C2=O)C(=O)N1] | 3-(4-((2-cyclopropylethyl)((1s,4s)-4- (4,4-difluoropiperidin-1- yl)cyclohexyl)amino)-1-oxoisoindolin- 2-yl)piperidine-2,6-dione |

In some embodiment, the compound of the present disclosure is for use in degrading and/or reducing casein kinase 1 alpha (CK1α).

In some embodiment, the compound of the present disclosure is for use in inhibiting casein kinase 1 alpha (CK1α) activity.

In some embodiment, the compound of the present disclosure is for use in preventing or treating blood cancer.

In some embodiment, the compound of the present disclosure is for manufacture of medicament for treatment of blood cancer.

In some embodiment, the present disclosure provides a pharmaceutical composition comprising: the compound of the present disclosure (represented by Formula (I), (I-a), (I-b) or (I-c)), or the pharmaceutically acceptable salt, enantiomer, stereoisomer or prodrug thereof, and optionally a pharmaceutically acceptable excipient or carrier.

In some embodiment, the pharmaceutical composition is for use in degrading and/or reducing casein kinase 1 alpha (CK1α).

In some embodiment, the pharmaceutical composition is for use in inhibiting casein kinase 1 alpha (CK1α) activity.

In some embodiment, the pharmaceutical composition is for use in preventing or treating blood cancer.

In some embodiment, the blood cancer is leukemia, lymphoma, or myeloma.

The present disclosure also provides a method of degrading and/or reducing casein kinase 1 alpha (CK1α) in a subject in need thereof, the method comprising administering to the subject an effective amount of the pharmaceutical composition of the present disclosure.

The present disclosure also provides a method of inhibiting casein kinase 1 alpha (CK1α) activity in a subject in need thereof, the method comprising administering to the subject an effective amount of the pharmaceutical composition of the present disclosure.

The present disclosure also provides a method of preventing or treating blood cancer in a subject in need thereof, the method comprising administering to the subject an effective amount of the pharmaceutical composition of the present disclosure.

In some embodiment, the blood cancer is leukemia, lymphoma, or myeloma.

In some embodiment, the leukemia is selected from the group consisting of acute myeloid leukemia (AML), acute lymphoblastic leukemia or acute lymphocytic leukemia (ALL), T-cell acute lymphoblastic leukemia (T-ALL), B-cell acute lymphoblastic leukemia (B-ALL), chronic myeloid leukemia (CML), chronic lymphocytic leukemia (CLL), hairy cell leukemia (HCL), T-cell prolymphocytic leukemia (T-PLL), Large granular lymphocytic leukemia, adult T-cell leukemia, chronic eosinophilic leukemia (CEL) and myelodysplastic syndrome (MDS).

In some embodiment, the lymphoma is selected from the group consisting of Hodgkin's lymphoma, diffuse large B-cell lymphoma (DLBCL), follicular lymphoma, small lymphocytic lymphoma (SLL), mantle cell lymphoma (MCL), marginal zone lymphomas, Burkitt lymphoma, lymphoplasmacytic lymphoma, primary central nervous system (CNS) lymphoma and peripheral T-cell lymphoma.

In some embodiment, the myeloma is selected from the group consisting of multiple myeloma, light chain myeloma, non-secretory myeloma, solitary plasmacytoma, extramedullary plasmacytoma, monoclonal gammopathy of undetermined significance (MGUS), smoldering multiple myeloma (SMM), immunoglobulin D (IgD) myeloma and immunoglobulin E (IgE) myeloma.

### DETAILED DESCRIPTION OF THE INVENTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

The present disclosure describes novel compounds which are represented by Formula (I) as follows, or a pharmaceutically acceptable salt, enantiomer, stereoisomer or prodrug thereof. In addition, the present disclosure describes an analog or a radical form of compounds represented by Formula (I).

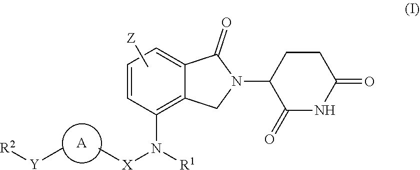

In Formula (I), in some embodiment, R¹ is C₁-C₈ alkyl, C₃-C₅ alkenyl, C₃-C₅ alkynyl, C₃-C₈ cycloalkyl or C₅-C₁₀ bicycloalkyl each optionally substituted with one or more halogen, OH, O—C₁-C₃ alkyl, NH₂, C₁-C₃ haloalkyl, optionally substituted C₆-C₁₀ aryl, C₁-C₃ alkyl, optionally substituted C₃-C₅ cycloalkyl, optionally substituted C₅-C₁₀ bicycloalkyl or optionally substituted C₃-C₆ heterocycloalkyl.

In some embodiment, X is a bond or —(CH₂)ₙ— optionally substituted with one or more halogen, OH, O—C₁-C₃ alkyl, C₁-C₃ haloalkyl, C₃-C₆ cycloalkyl or C₁-C₆ alkyl, wherein the C₁-C₆ alkyl group may be taken together with the atom to which it is attached to form a C₃-C₆ spiro alkyl ring, and wherein the —(CH₂)ₙ— group may contain 0-1 double bond or triple bond.

In some embodiment, n is an integer from 1 to 6.

In some embodiment, A is a bond, C₄-C₅ cycloalkyl, C₅-C₁₀ bicycloalkyl, C₅-C₁₀ heterobicycloalkyl or C₃-C₅ heterocycloalkyl each optionally substituted with one of more halogen, OH, O—C₁-C₃ alkyl, CN, C₁-C₃ haloalkyl, C₁-C₃ alkyl or C₃-C₅ cycloalkyl.

In some embodiment, Y is a bond or a —(CH₂)ₘ—.

In some embodiment, m is an integer of 1 or 2.

In some embodiment, Z is H or halogen.

In some embodiment, R² is —NR¹²R⁵, —NHC(O)R⁶, —NHC(O)NR⁸(R⁹), —C(O)NHR⁵, five or six-membered heteroaryl, or C₃-C₈ heterocycloalkyl or C₅-C₁₀ heterobicycloalkyl ring optionally substituted with one of more halogen, OH, O—C₁-C₃ alkyl, CN, C₁-C₃ haloalkyl, C₁-C₃ alkyl or C₃-C₅ cycloalkyl.

In some embodiment, R⁵ is H, C₁-C₅ alkyl, C₃-C₇ cycloalkyl, five or six-membered heteroaryl, C₅-C₁₀ bicycloalkyl, (C₃-C₆ cycloalkyl)-C₁-C₃ alkyl, (C₃-C₆ heterocycloalkyl)-C₁-C₃ alkyl, (C₆-C₁₀ aryl)-C₁-C₃ alkyl, (C₁-C₅-heteroaryl)-C₁-C₃ alkyl or C₃-C₇ heterocycloalkyl each optionally substituted with one or more halogen, CN, OH, O—(C₁-C₃ haloalkyl), O—(C₁-C₃ alkyl), C₁-C₃ haloalkyl, CH₃SO₂—, optionally substituted C₃-C₆ cycloalkyl, optionally substituted C₃-C₇ heterocycloalkyl or C(O)NR⁸(R⁹).

In some embodiment, R⁶ is C₁-C₆ alkyl, C₃-C₆ cycloalkyl or C₃-C₆ heterocycloalkyl each optionally substituted with one or more halogen, C₁-C₃ haloalkyl, optionally substituted C₃-C₆ cycloalkyl, or NR⁸(R⁹).

In some embodiment, R⁸ and R⁹ are each independently H, substituted C₁-C₃ alkyl or taken together with the nitrogen to which they are attached to form a 4-6 membered heterocyclic ring optionally substituted with one or more halogen or C₁-C₃ haloalkyl.

In some embodiment, R¹² is H, C₁-C₅ alkyl, or taken together with R⁵ and the nitrogen to which it is attached to form (i) C₃-C₅ cycloalkyl or heterocycloalkyl ring or (ii) C₅-C₁₀ bicycloalkyl or heterobicycloalkyl ring, each optionally substituted with one or more halogen, C₁-C₃ haloalkyl, OH, O—(C₁-C₃ alkyl), O—(C₁-C₃ haloalkyl) or CN.

In some embodiment, each stereocenter in the compound of Formula (I) is independently the R-enantiomer, the S-enantiomer or a mixture of R- and S-enantiomers.

In some embodiment, each double bond in the compound of Formula (I) is independently cis or trans.

In some embodiment, the compound is represented by Formula (I-a),

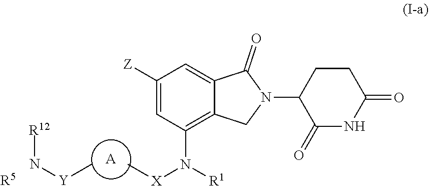

or a pharmaceutically acceptable salt, enantiomer, stereoisomer or prodrug thereof.

In Formula (I-a), in some embodiment, X is a bond or —(CH₂)ₙ—.

In some embodiment, R¹ is C₁-C₈ alkyl or C₃-C₅ cycloalkyl each optionally substituted with one or more halogen, OH, NH₂, C₁-C₃ alkyl, C₁-C₃ haloalkyl, O—(C₁-C₃ alkyl), optionally substituted C₆ aryl, optionally substituted C₃-C₅ cycloalkyl, optionally substituted C₃-C₆ heterocycloalkyl or optionally substituted C₆-C₅ bicycloalkyl.

In some embodiment, A is C₄-C₆ cycloalkyl, C₄-C₆ heterocycloalkyl or C₅-C₇ bicycloalkyl each optionally substituted with one or more halogen, OH, C₁-C₃ alkyl or C₁-C₃ haloalkyl.

In some embodiment, Y is a bond or —CH₂—.

In some embodiment, R⁵ is H, C₁-C₅ alkyl, C₃-C₆ cycloalkyl, 6-membered heteroaryl, C₅ bicycloalkyl, (C₃-C₄ cycloalkyl)-C₁-C₂ alkyl, (C₃-C₄ heterocycloalkyl)-C₁-C₂ alkyl, (C₆ aryl)-C₁-C₂ alkyl, (C₅ heteroaryl)-C₁-C₂ alkyl or C₃-C₄ heterocycloalkyl each optionally substituted with one or more halogen, CN, OH, O—(C₁-C₃ haloalkyl), O—(C₁-C₃ alkyl), C₁-C₃ haloalkyl, CH₃SO₂— or C(O)NR⁸(R⁹).

In some embodiment, R¹² is H, C₁-C₅ alkyl, or taken together with R⁵ and the nitrogen to which it is attached to form a C₃-C₅ heterocycloalkyl ring optionally substituted with one or more halogen or C₁-C₃ haloalkyl.

In some embodiment, the compound is represented by Formula (I-b),

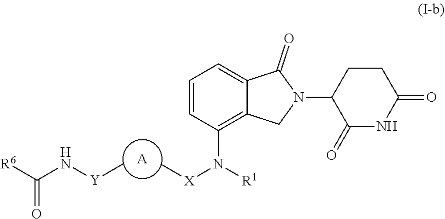

or a pharmaceutically acceptable salt, enantiomer, stereoisomer or prodrug thereof.

In Formula (I-b), in some embodiment, X is a bond or —(CH₂)ₙ—.

In some embodiment, R¹ is C₃-C₆ alkyl optionally substituted with one or more halogen, OH, O—C₁-C₃ alkyl, NH₂, C₁-C₃ haloalkyl.

In some embodiment, A is bond or C₄-C₆ cycloalkyl optionally substituted with one or more halogen, OH, O—C₁-C₃ alkyl, NH₂, C₁-C₃ haloalkyl.

In some embodiment, Y is a bond.

In some embodiment, R⁶ is C₁-C₆ alkyl, optionally substituted with NR⁸(R⁹).

In some embodiment, the compound is represented by Formula (I-c),

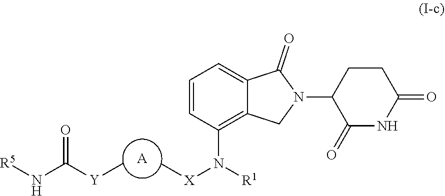

or a pharmaceutically acceptable salt, enantiomer, stereoisomer or prodrug thereof.

In Formula (I-c), in some embodiment, X is a bond or —(CH₂)ₙ—.

In some embodiment, R¹ is C₁-C₆ alkyl optionally substituted with optionally substituted C₃-C₈ cycloalkyl or optionally substituted C₃-C₆ heterocycloalkyl.

In some embodiment, A is bond, C₄-C₆ cycloalkyl, C₄-C₆ heterocycloalkyl or C₅-C₇ bicycloalkyl each optionally substituted with one or more halogen, OH, O—C₁-C₃ alkyl, NH₂, C₁-C₃ haloalkyl.

In some embodiment, Y is a bond.

In some embodiment, R⁵ is H or C₁-C₃ alkyl.

In some embodiment, the compound is represented by Formula (I-d),

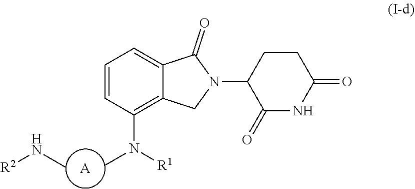

or a pharmaceutically acceptable salt, enantiomer, stereoisomer or prodrug thereof.

In Formula (I-d), in some embodiment, R¹ is C₁-C₃ alkyl optionally substituted with optionally substituted C₃-C₆ cycloalkyl. For example, C₃-C₆ cycloalkyl can be substituted with one or more halogen, CN, OH, C₁-C₃ alkyl, O—(C₁-C₃ haloalkyl), O—(C₁-C₃ alkyl) or C₁-C₃ haloalkyl.

In some embodiment, A is optionally substituted C₄-C₆ cycloalkyl. For example, A can be substituted with one or more halogen, CN, OH, C₁-C₃ alkyl, O—(C₁-C₃ haloalkyl), 0-(C₁-C₃ alkyl) or C₁-C₃ haloalkyl.

In some embodiment, R² is C₁-C₄ alkyl or (C₃-C₄ cycloalkyl)-C₁-C₂ alkyl, each optionally substituted with one or more halogen, CN, OH, O—(C₁-C₃ haloalkyl), O—(C₁-C₃ alkyl), C₁-C₃ haloalkyl or CH₃SO₂—. For example, R² is C₁-C₃ alkyl optionally substituted with one or more halogen, O—(C₁-C₃ haloalkyl), O—(C₁-C₃ alkyl) or C₁-C₃ haloalkyl.

In Formula (I), In some embodiment, each X and Y is a bond; A is C₄-C₆ cycloalkyl; R¹ is C₁-C₃ alkyl optionally substituted with optionally substituted C₃-C₆ cycloalkyl; and R² is 5-membered heteroaryl.

The present disclosure describes novel compounds which are represented by Formula (I) as follows, or a pharmaceutically acceptable salt, enantiomer, stereoisomer or prodrug thereof. In addition, the present disclosure describes an analog or a radical form of compounds represented by Formula (I).

As used herein, the term “compound” unless otherwise indicated, refers to any specific chemical compound disclosed herein and includes tautomers, regioisomers, geometric isomers, and where applicable, stereoisomers, including optical isomers (enantiomers) and other stereoisomers (diastereomers) thereof, as well as pharmaceutically acceptable salts and derivatives (including prodrug forms) thereof where applicable, in context. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, in context to prodrug forms of compounds which have been modified to facilitate the administration and delivery of compounds to a site of activity. It is noted that in describing the present compounds, numerous substituents and variables associated with same, among others, are described. It is understood by those of ordinary skill that molecules which are described herein are stable compounds as generally described hereunder.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, or may be stereoisomeric mixtures, and include all diastereomeric, and enantiomeric forms. In addition, it is understood that, in any compound described herein having one or more double bond(s) generating geometrical isomers that can be defined as E or Z, each double bond may independently be E or Z a mixture thereof. Stereoisomers are obtained, if desired, by methods such as, stereoselective synthesis and/or the separation of stereoisomers by chiral chromatographic columns. Likewise, it is understood that, in any compound described, all tautomeric and conformeric forms are also intended to be included. A conformer is a structure that is a conformational isomer. Conformational isomerism is the phenomenon of molecules with the same structural formula but different conformations (conformers) of atoms about a rotating bond.

As used herein, any “R” group(s) represent substituents that can be attached to the indicated atom. An R group may be substituted or unsubstituted. Whenever a group is described as being “optionally substituted” that group may be unsubstituted or substituted with one or more of the indicated substituents. Likewise, when a group is described as being “unsubstituted or substituted” if substituted, the substituent may be selected from one or more the indicated substituents. If no substituents are indicated, it is meant that the indicated “optionally substituted” or “substituted” group may be individually and independently substituted with one or more group(s) individually and independently selected from alkyl, cycloalkyl, aryl, heteroaryl, heterocyclyl, aralkyl, heteroaralkyl, heterocyclyl(alkyl), hydroxy, alkoxy, cycloalkoxy, aryloxy, acyl, mercapto, alkylthio, arylthio, cyano, halogen, C-amido, N-amido, C-carboxy, O-carboxy, isocyanato, thiocyanato, isothiocyanato, nitro, haloalkyl, haloalkoxy, amino (including mono-substituted amino and di-substituted amino), and alkylamino. When a group is not described as “optionally substituted,” “unsubstituted” or “substituted,” such group is unsubstituted unless the definition of such group states otherwise.

As used herein, “Cₐ to C_{b}” or “Cₐ-C_{b}” in which “a” and “b” are integers refer to the number of carbon atoms in an alkyl, alkenyl or alkynyl group, or the number of carbon atoms in the ring of a cycloalkyl, aryl, heteroaryl, heterocycloalkyl or heterocyclyl group. That is, the alkyl, alkenyl, alkynyl, ring of the cycloalkyl, ring of the aryl, ring of the heterocycloalkyl, ring of the heteroaryl or ring of the heterocyclyl can contain from “a” to “b”, inclusive, carbon atoms.

As used herein, the term “alkyl” refers to a saturated monovalent chain of carbon atoms, which may be optionally branched. It is understood that in embodiments that include alkyl, illustrative variations of those embodiments include lower alkyl, such as C₁-C₈, C₁-C₆, C₁-C₅, C₁-C₄, C₁-C₃ alkyl, methyl, ethyl, propyl, 3-methylpentyl, and the like.

As used herein, the term “alkenyl” refers to a straight chain or branched hydrocarbon having at least 2 carbon atoms and at least one double bond. Alkenyl can include any number of carbons, such as C₂, C₂-C₃, C₂-C₄, C₂-C₅, C₂-C₆, C₂-C₇, C₂-C₈, C₃, C₃-C₄, C₃-C₅, C₃-C₆, C₃-C₇, C₃-C₈, C₄, C₄-C₅, C₄-C₆, C₄-C₇, C₄-C₈, C₅, C₅-C₆, C₅-C₇, C₅-C₈, C₆, C₆-C₇, C₆-C₈, C₇, C₇-C₈, and C₈. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1.4-pentadienyl, 1-hexenyl, 2-hexenyl, 3-hexenyl, 1,3-hexadienyl, 1,4-hexadienyl, 1.5-hexadienyl, 2,4-hexadienyl, or 1,3,5-hexatrienyl. Alkenyl groups can be optionally substituted with one or more moieties selected from halo, hydroxy, amino, alkylamino, alkoxy, haloalkyl, carboxy, amido, nitro, oxo, and cyano.

As used herein, the term “alkynyl” refers to either a straight chain or branched hydrocarbon having at least 2 carbon atoms and at least one triple bond. Alkynyl can include any number of carbons, such as C₂, C₂-C₃, C₂-C₄, C₂-C₅, C₂-C₆, C₂-C₇, C₂-C₈, C₃, C₃-C₄, C₃-C₅, C₃-C₆, C₃-C₇, C₃-C₈, C₄, C₄-C₅, C₄-C₆, C₄-C₇, C₄-C₈, C₅, C₅-C₆, C₅-C₇, C₅-C₈, C₆, C₆-C₇, C₆-C₈, C₇, C₇-C₈, and C₈. Examples of alkynyl groups include, but are not limited to, acetylenyl, propynyl, 1-butynyl, 2-butynyl, isobutynyl, sec-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can be optionally substituted with one or more moieties selected from halo, hydroxy, amino, alkylamino, alkoxy, haloalkyl, carboxy, amido, nitro, oxo, and cyano.

As used herein, the term “haloalkyl” refers to an alkyl group in which one or more of the hydrogen atoms has been replaced by one or more halogen atom(s). The term “Cₙ₋ₘ haloalkyl” or “Cₙ-Cₘ haloalkyl” refers to a Cₙ₋ₘ alkyl group having n to m carbon atoms and from at least one up to {2(n to m)+1}halogen atoms, which may either be the same or different. In some embodiments, the halogen atoms are fluoro atoms. In some embodiments, the haloalkyl group has 1 to 6 or 1 to 4 carbon atoms. Example haloalkyl groups include CF₃, C₂F₅, CHF₂, CH₂F, CCl₃, CHCl₂, C₂Cl₅ and the like. In some embodiments, the haloalkyl group is a fluoroalkyl group.

As used herein, the term “cycloalkyl” refers to a monovalent chain of carbon atoms, a portion of which forms a ring. It is understood that in embodiments that include cycloalkyl, illustrative variations of those embodiments include lower cycloalkyl, such as C₃-C₈, C₃-C₇, C₃-C₆, C₃-C₅, C₄-C₈, C₄-C₇, C₄-C₆ cycloalkyl, cyclopropyl, cyclohexyl, 3-ethylcyclopentyl, and the like.

As used herein, the term “bicycloalkyl” refers to two cycloalkyl groups, defined hereinabove, connecting with each other to form a bridged, fused or spiro bicyclic compound.

As used herein, the term “fused bicycloalkyl” refers to two cycloalkyl groups which share two adjacent atoms. In other words, the rings share one covalent bond, i.e. the so-called bridgehead atoms are directly connected.

As used herein, the term “bridged bicycloalkyl” refers to two cycloalkyl groups of which moieties have more than two atoms in common. That is, two rings do not share adjacent carbon atoms, but share three or more atoms, separating the two bridgehead atoms by a bridge containing at least one atom. Examples include, but are not limited to, bicyclo[3.2.1]heptyl (“norbornyl”), bicyclo[2.2.2]octyl, and the like.

As used herein, the term “spirobicycloalkyl” refers to bicyclic groups in which the two rings are attached at a single carbon atom that is a member of each of the two rings. The term includes both spirobicycloalkyls, in which the two rings are cycloalkyl rings attached at a single carbon atom that is a member of each of the two rings, and spirobicycloheteroalkyls, in which one ring is a heterocyclyl ring and the other ring is a cycloalkyl ring attached at a single carbon atom that is a member of each of the two rings, or in which both rings are heterocyclyl rings attached at a single carbon atom that is a member of each of the two rings. Examples of spirobicyclyl groups include spiro[3.3]heptenyl, spiro[3.4]octanyl, azaspiro[3.3]heptanyl, oxaazaspiro[3.3]heptanyl, oxa-azaspiro[3.3]heptanyl, and azaspiro[3.4]octanyl.

As used herein, “heterocycloalkyl” refers to a non-aromatic ring, which has at least one heteroatom ring member independently selected from nitrogen, sulfur, and oxygen. Heterocycloalkyl groups can include mono- or polycyclic (e.g., fused, bridged, or spiro) ring systems. Heterocycloalkyl also includes one or more aromatic rings fused to the non-aromatic heterocycloalkyl ring. The ring-forming carbon atoms of a heterocycloalkyl group can be optionally substituted by oxo. The ring-forming heteroatoms of the heterocycloalkyl group can be oxidized to form an N-oxide or a sulfonyl group. It is understood that in embodiments that include heterocycloalkyl, illustrative variations of those embodiments include lower cycloalkyl, such as C₃-C₈, C₃-C₇, C₃-C₆, C₃-C₅, C₄-C₈, C₄-C₇, C₄-C₆ heterocycloalkyl and the like. Examples of heterocycloalkyl group include morpholine ring, pyrrolidine ring, piperazine ring, piperidine ring, tetrahydropyran ring, tetrahyropyridine, azetidine ring, tetrahydrofuran, etc.

As used herein, “heterobicycloalkyl” refers to a bicycloalkyl structure, which is unsubstituted or substituted, in which at least one carbon atom is replaced with a heteroatom independently selected from oxygen, nitrogen, and sulfur.

As used herein, the term “aryl” refers to an aromatic carbon ring system having any suitable number of ring atoms and any suitable number of rings. Aryl groups can include any suitable number of carbon ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be optionally substituted with one or more moieties selected from alkyl, alkenyl, alkynyl, haloalkyl, halo, hydroxy, amino, alkylamino, alkoxy, haloalkyl, carboxy, alkyl carboxylate, amido, nitro, oxo, and cyano.

As used herein, the term “heteroaryl” refers to substituted and unsubstituted aromatic 5- or 6-membered monocyclic groups and 9- or 10-membered bicyclic groups that have at least one heteroatom (0, S or N) in at least one of the rings, said heteroatom-containing ring preferably having 1, 2, or 3 heteroatoms independently selected from O, S, and/or N. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic group are aromatic and may contain only carbon atoms. The nitrogen and sulfur atoms may optionally be oxidized and the nitrogen atoms may optionally be quaternized. Bicyclic heteroaryl groups must include only aromatic rings. The heteroaryl group may be attached at any available nitrogen or carbon atom of any ring. The heteroaryl ring system may be unsubstituted or may contain one or more substituents. Examples of monocyclic heteroaryl are, but not limited to, thiazolyl, oxazolyl, thiophenyl, furanyl, pyrrolyl, imidazolyl, isoxazolyl, pyrazolyl, triazolyl, thiadiazolyl, tetrazolyl, oxadiazolyl, pyridinyl, pyridazinyl, pyrimidinyl, pyrazinyl, thiazolyl, and other similar groups. Examples of bicyclic heteroaryl are, but not limited to, indolyl, benzothiophenyl, benzofuranyl, benzimidazolyl, benzoxazolyl, benzisoxazolyl, benzthiazolyl, benzthiadiazolyl, benztriazolyl, quinolinyl, isoquinolinyl, purinyl, puropyridinyl, oxochromene, dioxoisoindolin, pyrazolopyridinyl, pyrazolo[1,5-a]pyridinyl, and other similar groups.

In some embodiment, the compound is selected from the following Table A.

TABLE A

| Com- | | |
|---|---|---|
| pounds | | |
| No. | Structure | Name |
| 2 | 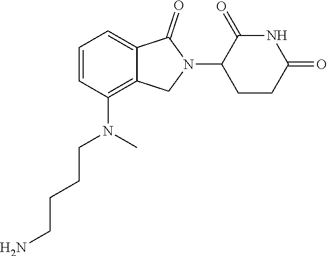 [SMILES: CN(CCCCN)c1cccc2c1CN(C1CCC(=O)NC1=O)C2=O] | 3-(4-((4-aminobutyl)(methyl)amino)-1- oxoisoindolin-2-yl)piperidine-2,6- dione |
| 3 | 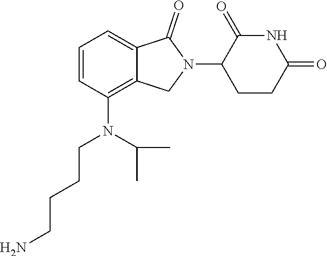 [SMILES: CC(C)N(CCCCN)c1cccc2c1CN(C1CCC(=O)NC1=O)C2=O] | 3-(4-((4- aminobutyl)(isopropyl)amino)-1- oxoisoindolin-2-yl)piperidine-2,6- dione |
| 4 | 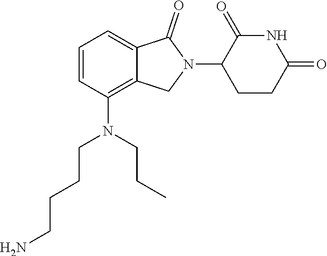 [SMILES: CCCN(CCCCN)c1cccc2c1CN(C1CCC(=O)NC1=O)C2=O] | 3-(4-((4-aminobutyl)(propyl)amino)-1- oxoisoindolin-2-yl)piperidine-2,6- dione |
| 46 | 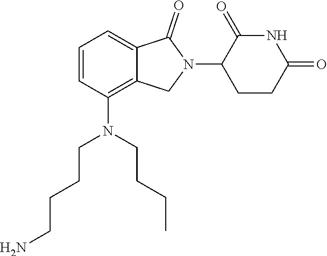 [SMILES: CCCCN(CCCCN)c1cccc2c1CN(C1CCC(=O)NC1=O)C2=O] | 3-(4-((4-aminobutyl)(butyl)amino)-1- oxoisoindolin-2-yl)piperidine-2,6- dione |
| 47 | 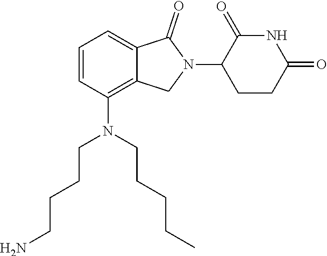 [SMILES: CCCCCN(CCCCN)c1cccc2c1CN(C1CCC(=O)NC1=O)C2=O] | 3-(4-((4-aminobutyl)(pentyl)amino)-1- oxoisoindolin-2-yl)piperidine-2,6- dione |
| 48 | 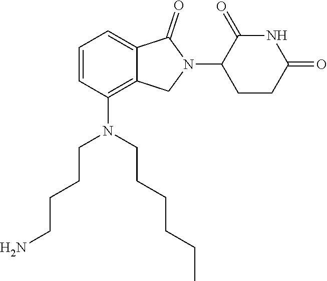 [SMILES: CCCCCCN(CCCCN)c1cccc2c1CN(C1CCC(=O)NC1=O)C2=O] | 3-(4-((4-aminobutyl)(hexyl)amino)-1- oxoisoindolin-2-yl)piperidine-2,6- dione |
| 49 | 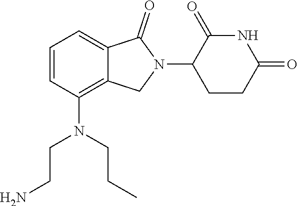 [SMILES: CCCN(CCN)c1cccc2c1CN(C1CCC(=O)NC1=O)C2=O] | 3-(4-((2-aminoethyl)(propyl)amino)-1- oxoisoindolin-2-yl)piperidine-2,6- dione |
| 50 | 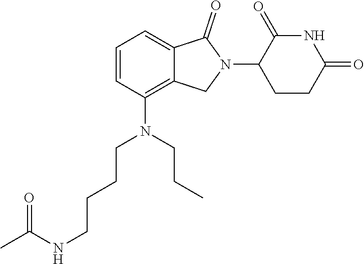 [SMILES: CCCN(CCCCNC(C)=O)c1cccc2c1CN(C1CCC(=O)NC1=O)C2=O] | N-(4-((2-(2,6-dioxopiperidin-3-yl)-1- oxoisoindolin-4- yl)(propyl)amino)butyl)acetamide |
| 51 | 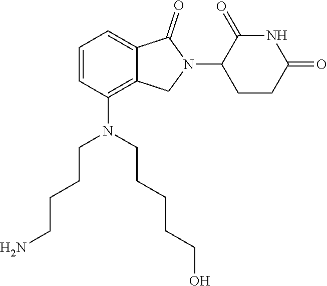 [SMILES: NCCCCN(CCCCCO)c1cccc2c1CN(C1CCC(=O)NC1=O)C2=O] | 3-(4-((4-aminobutyl)(5- hydroxypentyl)amino)-1- oxoisoindolin-2-yl)piperidine-2,6- dione |
| 52 | 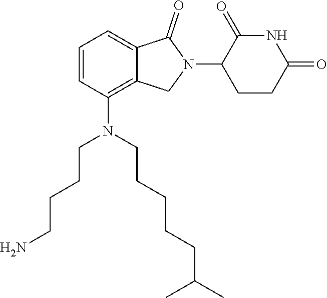 [SMILES: CC(C)CCCCCN(CCCCN)c1cccc2c1CN(C1CCC(=O)NC1=O)C2=O] | 3-(4-((4-aminobutyl)(6- methylheptyl)amino)-1- oxoisoindolin-2-yl)piperidine-2,6- dione |
| 53 | 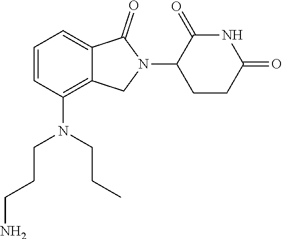 [SMILES: CCCN(CCCN)c1cccc2c1CN(C1CCC(=O)NC1=O)C2=O] | 3-(4-((3-aminopropyl(propyl)amino)-1- oxoisoindolin-2-yl)piperidine-2,6- dione |
| 55 | 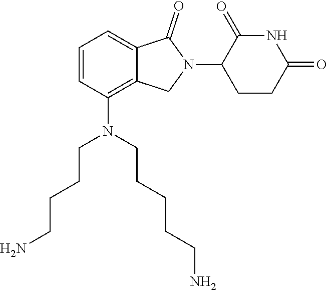 [SMILES: NCCCCCN(CCCCN)c1cccc2c1CN(C1CCC(=O)NC1=O)C2=O] | 3-(4-((4-aminobutyl)(5- aminopentyl)amino)-1-oxoisoindolin- 2-yl)piperidine-2,6-dione |
| 56 | 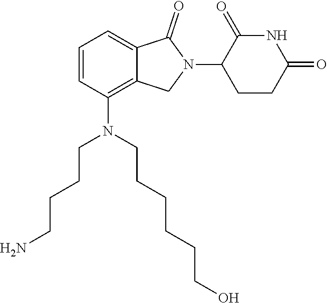 [SMILES: NCCCCN(CCCCCCO)c1cccc2c1CN(C1CCC(=O)NC1=O)C2=O] | 3-(4-((4-aminobutyl)(6- hydroxyhexyl)amino)-1-oxoisoindolin- 2-yl)piperidine-2,6-dione |
| 57 | 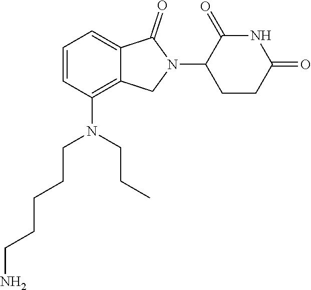 [SMILES: CCCN(CCCCCN)c1cccc2c1CN(C1CCC(=O)NC1=O)C2=O] | 3-(4-((5-aminopentyl)(propyl)amino)- 1-oxoisoindolin-2-yl)piperidine-2,6-dione |
| 60 | 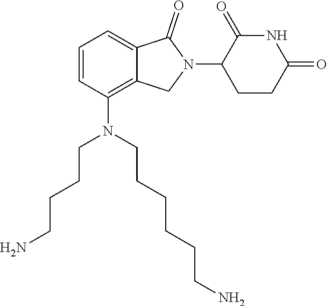 [SMILES: NCCCCCCN(CCCCN)c1cccc2c1CN(C1CCC(=O)NC1=O)C2=O] | 3-(4-((4-aminobutyl)(6- aminohexyl)amino)-1-oxoisoindolin-2- yl)piperidine-2,6-dione |
| 63 | 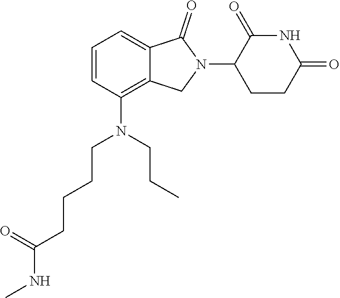 [SMILES: CCCN(CCCCC(=O)NC)c1cccc2c1CN(C1CCC(=O)NC1=O)C2=O] | 5-((2-(2,6-dioxopiperidin-3-yl)-1- oxoisoindolin-4-yl)(propyl)amino)-N- methylpentanamide |
| 66 | 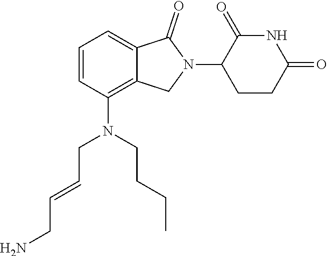 [SMILES: CCCCN(C/C=C/CN)c1cccc2c1CN(C1CCC(=O)NC1=O)C2=O] | (E)-3-(4-((4-aminobut-2-en-1- yl)(butyl)amino)-1-oxoisoindolin-2- yl)piperidine-2,6-dione |
| 67 | 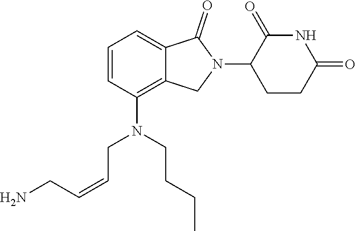 [SMILES: CCCCN(C/C=C\CN)c1cccc2c1CN(C1CCC(=O)NC1=O)C2=O] | (Z)-3-(4-((4-aminobut-2-en-1- yl)(butyl)amino)-1-oxoisoindolin-2- yl)piperidine-2,6-dione |
| 68 | 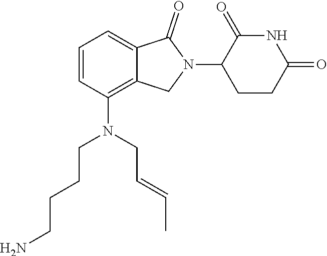 [SMILES: C/C=C/CN(CCCCN)c1cccc2c1CN(C1CCC(=O)NC1=O)C2=O] | (E)-3-(4-((4-aminobutyl)(but-2-en-1- yl)amino)-1-oxoisoindolin-2- yl)piperidine-2,6-dione |
| 69 | 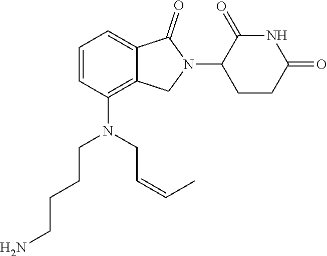 [SMILES: C/C=C\CN(CCCCN)c1cccc2c1CN(C1CCC(=O)NC1=O)C2=O] | (Z)-3-(4-((4-aminobutyl)(but-2-en-1- yl)amino)-1-oxoisoindolin-2- yl)piperidine-2,6-dione |
| 80 | 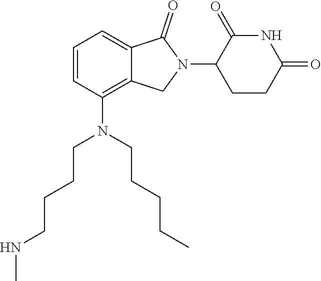 [SMILES: CCCCCN(CCCCNC)c1cccc2c1CN(C1CCC(=O)NC1=O)C2=O] | 3-(4-((4- (methylamino)butyl)(pentyl)amino)-1- oxoisoindolin-2-yl)piperidine-2,6- dione |
| 81 | 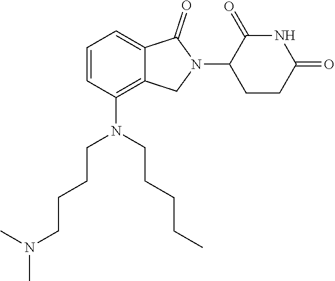 [SMILES: CCCCCN(CCCCN(C)C)c1cccc2c1CN(C1CCC(=O)NC1=O)C2=O] | 3-(4-((4- (dimethylamino)butyl)(pentyl)amino)- 1-oxoisoindolin-2-yl)piperidine-2,6-dione |
| 82 | 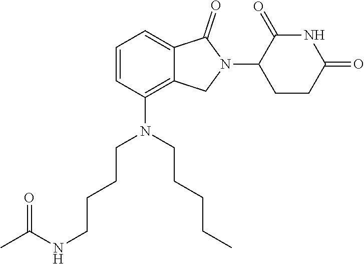 [SMILES: CCCCCN(CCCCNC(C)=O)c1cccc2c1CN(C1CCC(=O)NC1=O)C2=O] | N-(4-((2-(2,6-dioxopiperidin-3-yl)-1- oxoisoindolin-4- yl)(pentyl)amino)butyl)acetamide |
| 87 | 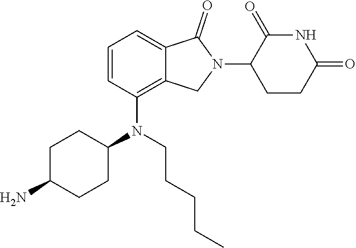 [SMILES: CCCCCN(c1cccc2c1CN(C1CCC(=O)NC1=O)C2=O)[C@H]1CC[C@@H](N)CC1] | 3-(4-(((1S,4s)-4- aminocyclohexyl)(pentyl)amino)-1- oxoisoindolin-2-yl)piperidine-2,6- dione |
| 88 | 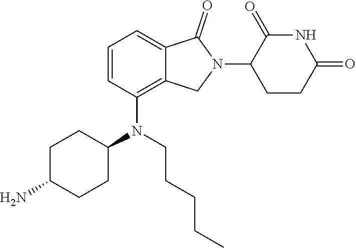 [SMILES: CCCCCN(c1cccc2c1CN(C1CCC(=O)NC1=O)C2=O)[C@H]1CC[C@H](N)CC1] | 3-(4-(((1r,4r)-4- aminocyclohexyl)(pentyl)amino)-1- oxoisoindolin-2-yl)piperidine-2,6- dione |
| 89 | 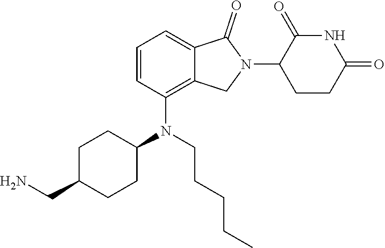 [SMILES: CCCCCN(c1cccc2c1CN(C1CCC(=O)NC1=O)C2=O)[C@H]1CC[C@@H](CN)CC1] | 3-(4-(((1s,4s)-4- (aminomethyl)cyclohexyl)(pentyl)amino)- 1-oxoisoindolin-2-yl)piperidine-2,6-dione |
| 90 | 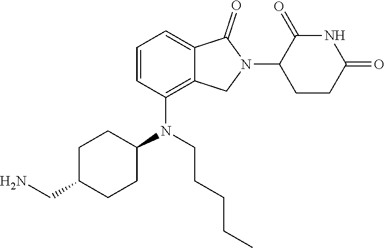 [SMILES: CCCCCN(c1cccc2c1CN(C1CCC(=O)NC1=O)C2=O)[C@H]1CC[C@H](CN)CC1] | 3-(4-(((1r,4r)-4- (aminomethyl)cyclohexyl)(pentyl)amino)- 1-oxoisoindolin-2-yl)piperidine-2,6- dione |
| 91 | 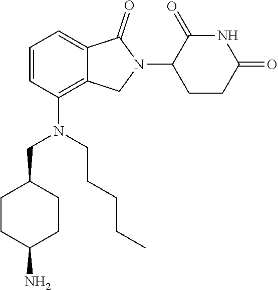 [SMILES: CCCCCN(C[C@H]1CC[C@@H](N)CC1)c1cccc2c1CN(C1CCC(=O)NC1=O)C2=O] | 3-(4-((((1s,4s)-4- aminocyclohexyl)methyl)(pentyl)amino)-1- oxoisoindolin-2-yl)piperidine-2,6-dione |
| 92 | 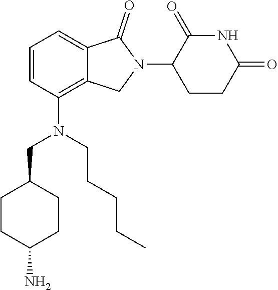 [SMILES: CCCCCN(C[C@H]1CC[C@H](N)CC1)c1cccc2c1CN(C1CCC(=O)NC1=O)C2=O] | 3-(4-((((1r,4r)-4- aminocyclohexyl)methyl)(pentyl)amino)- 1-oxoisoindolin-2-yl)piperidine-2,6-dione |
| 93 | 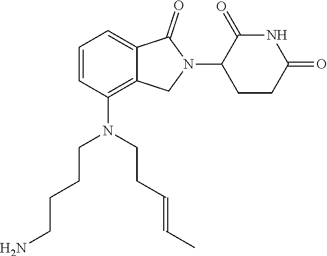 [SMILES: C/C=C/CCN(CCCCN)c1cccc2c1CN(C1CCC(=O)NC1=O)C2=O] | (E)-3-(4-((4-aminobutyl)(pent-3-en-1- yl)amino)-1-oxoisoindolin-2- yl)piperidine-2,6-dione |
| 94 | 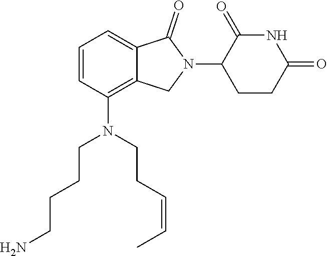 [SMILES: C/C=C\CCN(CCCCN)c1cccc2c1CN(C1CCC(=O)NC1=O)C2=O] | (Z)-3-(4-((4-aminobutyl)(pent-3-en-1- yl)amino)-1-oxoisoindolin-2- yl)piperidine-2,6-dione |
| 95 | 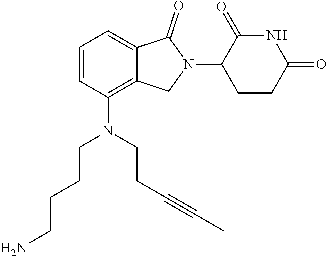 [SMILES: CC#CCCN(CCCCN)c1cccc2c1CN(C1CCC(=O)NC1=O)C2=O] | 3-(4-((4-aminobutyl)(pent-3-yl-1- yl)amino)-1-oxoisoindolin-2- yl)piperidine-2,6-dione |
| 96 | 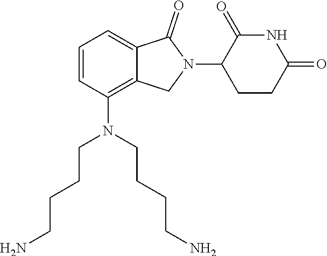 [SMILES: NCCCCN(CCCCN)c1cccc2c1CN(C1CCC(=O)NC1=O)C2=O] | 3-(4-(bis(4-aminobutyl)amino)-1- oxoisoindolin-2-yl)piperidine-2,6-dione |
| 97 | 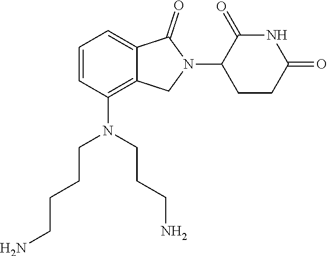 [SMILES: NCCCCN(CCCN)c1cccc2c1CN(C1CCC(=O)NC1=O)C2=O] | 3-(4-((4-aminobutyl)(3- aminopropyl)amino)-1-oxoisoindolin- 2-yl)piperidine-2,6-dione |
| 102 | 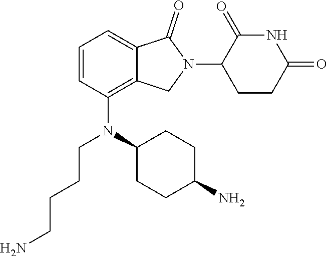 [SMILES: NCCCCN(c1cccc2c1CN(C1CCC(=O)NC1=O)C2=O)[C@H]1CC[C@@H](N)CC1] | 3-(4-((4-aminobutyl)((1s,4s)-4- aminocyclohexyl)amino)-1- oxoisoinodolin-2-yl)piperidine-2,6-dione |
| 103 | 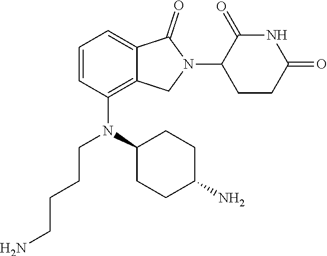 [SMILES: NCCCCN(c1cccc2c1CN(C1CCC(=O)NC1=O)C2=O)[C@H]1CC[C@H](N)CC1] | 3-(4-((4-aminobutyl)((1r,4r)-4- aminocyclohexyl)amino)-1- oxoisoindolin-2-yl)piperidine-2,6-dione |
| 104 | 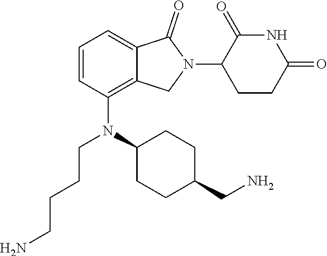 [SMILES: NCCCCN(c1cccc2c1CN(C1CCC(=O)NC1=O)C2=O)[C@H]1CC[C@@H](CN)CC1] | 3-(4-((4-aminobutyl)((1s,4s)-4- (aminomethyl)cyclohexyl)amino)-1- oxoisoindolin-2-yl)piperidine-2,6-dione |
| 105 | 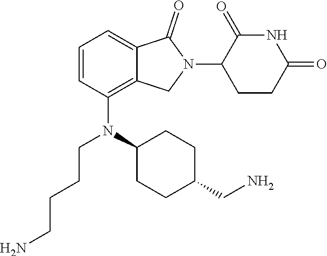 [SMILES: NCCCCN(c1cccc2c1CN(C1CCC(=O)NC1=O)C2=O)[C@H]1CC[C@H](CN)CC1] | 3-(4-((4-aminobutyl)((1r,4r)-4- (aminomethyl)cyclohexyl)amino)-1- oxoisoindolin-2-yl)piperidine-2,6- dione |
| 106 | 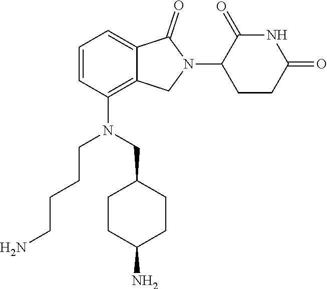 [SMILES: NCCCCN(C[C@H]1CC[C@@H](N)CC1)c1cccc2c1CN(C1CCC(=O)NC1=O)C2=O] | 3-(4-((4-aminobutyl)(((1s,4s)-4- aminocyclohexyl)methyl)amino)-1- oxoisoindolin-2-yl)piperidine-2,6-dione |
| 107 | 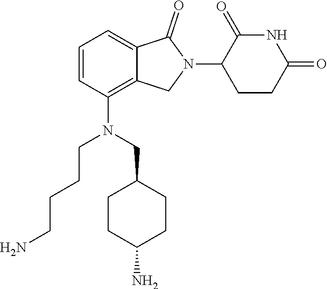 [SMILES: NCCCCN(C[C@H]1CC[C@H](N)CC1)c1cccc2c1CN(C1CCC(=O)NC1=O)C2=O] | 3-(4-((4-aminobutyl)(((1r,4r)-4- aminocyclohexyl)methyl)amino)-1- oxoisoindolin-2-yl)piperidine-2,6-dione |
| 108 | 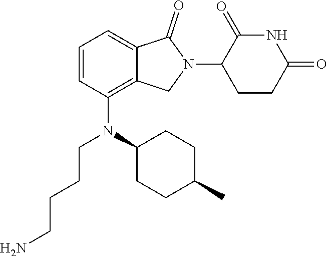 [SMILES: C[C@H]1CC[C@@H](N(CCCCN)c2cccc3c2CN(C2CCC(=O)NC2=O)C3=O)CC1] | 3-(4-((4-aminobutyl)((1s,4s)-4- methylcyclohexyl)amino)-1- oxoisoindolin-2-yl)piperidine-2,6-dione |
| 109 | 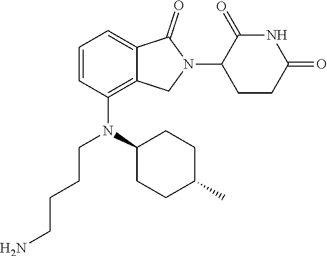 [SMILES: C[C@H]1CC[C@H](N(CCCCN)c2cccc3c2CN(C2CCC(=O)NC2=O)C3=O)CC1] | 3-(4-((4-aminobutyl)((1r,4r)-4- methylcyclohexyl)amino)-1- oxoisoindolin-2-yl)piperidine-2,6-dione |
| 115 | 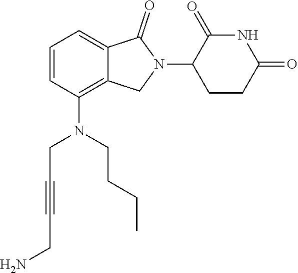 [SMILES: CCCCN(CC#CCN)c1cccc2c1CN(C1CCC(=O)NC1=O)C2=O] | 3-(4-((4-aminobut-2-yn-1- yl)(butyl)amino)-1-oxoisoindolin-2- yl)piperidine-2,6-dione |
| 116 | 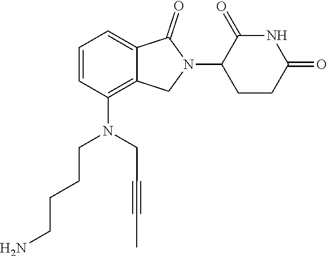 [SMILES: CC#CCN(CCCCN)c1cccc2c1CN(C1CCC(=O)NC1=O)C2=O] | 3-(4-((4-aminobutyl)(but-2-yn-1- yl)amino)-1-oxoisoindolin-2- yl)piperidine-2,6-dione |
| 123 | 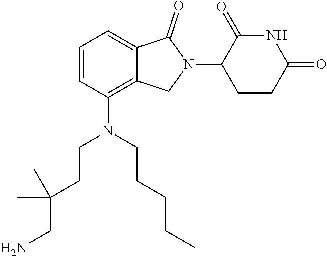 [SMILES: CCCCCN(CCC(C)(C)CN)c1cccc2c1CN(C1CCC(=O)NC1=O)C2=O] | 3-(4-((4-amino-3,3-dimethylbutyl)(pentyl) amino)-1- oxoisoindolin-2-yl)piperidine-2,6-dione |
| 124 | 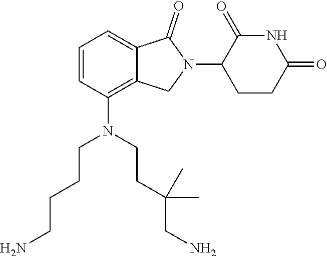 [SMILES: CC(C)(CN)CCN(CCCCN)c1cccc2c1CN(C1CCC(=O)NC1=O)C2=O] | 3-(4-((4-amino-3,3-dimethylbutyl)(4- aminobutyl)amino)-1-oxoisoindolin-2- yl)piperidine-2,6-dione |
| 125 | 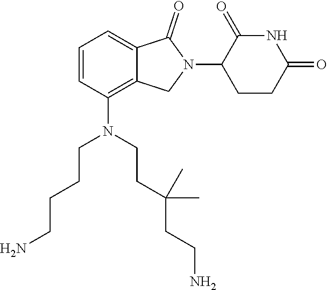 [SMILES: CC(C)(CCN)CCN(CCCCN)c1cccc2c1CN(C1CCC(=O)NC1=O)C2=O] | 3-(4-((5-amino-3,3-dimethylpentyl)(4- aminobutyl)amino)-1-oxoisoindolin-2- yl)piperidine-2,6-dione |
| 126 | 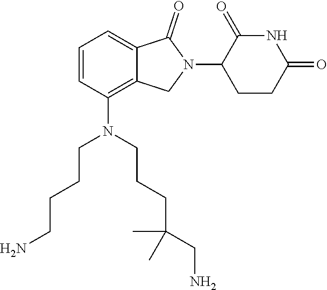 [SMILES: CC(C)(CN)CCCN(CCCCN)c1cccc2c1CN(C1CCC(=O)NC1=O)C2=O] | 3-(4-((5-amino-4,4-dimethylpentyl)(4- aminobutyl)amino)-1-oxoisoindolin-2- yl)piperidine-2,6-dione |
| 143 | 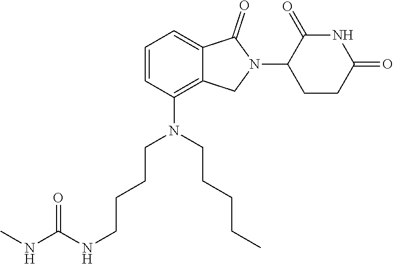 [SMILES: CCCCCN(CCCCNC(=O)NC)c1cccc2c1CN(C1CCC(=O)NC1=O)C2=O] | 1-(4-((2-(2,6-dioxopiperidin-3-yl)-1- oxoisoindolin-4- yl)(pentyl)amino)butyl)-3-methylurea |
| 148 | 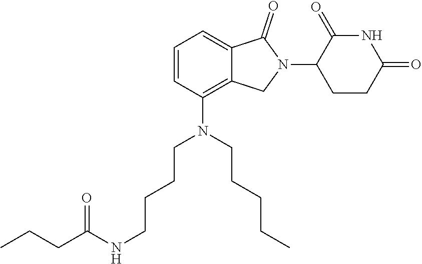 [SMILES: CCCCCN(CCCCNC(=O)CCC)c1cccc2c1CN(C1CCC(=O)NC1=O)C2=O] | N-(4-((2-(2,6-dioxopiperidin-3-yl)-1- oxoisoindolin-4- yl)(pentyl)amino)butyl)butyramide |
| 149 | 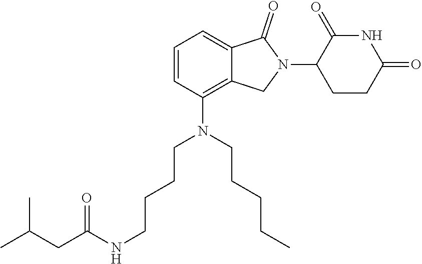 [SMILES: CCCCCN(CCCCNC(=O)CC(C)C)c1cccc2c1CN(C1CCC(=O)NC1=O)C2=O] | N-(4-((2-(2,6-dioxopiperidin-3-yl)-1- oxoisoindolin-4- yl)(pentyl)amino)butyl)-3- methylbutanamide |
| 150 | 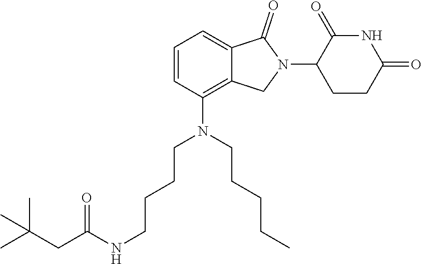 [SMILES: CCCCCN(CCCCNC(=O)CC(C)(C)C)c1cccc2c1CN(C1CCC(=O)NC1=O)C2=O] | N-(4-((2-(2,6-dioxopiperidin-3-yl)-1- oxoisoindolin-4- yl)(pentyl)amino)butyl)-3,3- dimethylbutanamide |
| 151 | 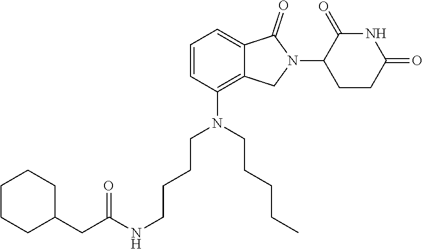 [SMILES: CCCCCN(CCCCNC(=O)CC1CCCCC1)c1cccc2c1CN(C1CCC(=O)NC1=O)C2=O] | 2-cyclohexyl-N-(4-((2-(2,6- dioxopiperidin-3-yl)-1-oxoisoindolin- 4-yl)(pentyl)amino)butyl)acetamide |
| 163 | 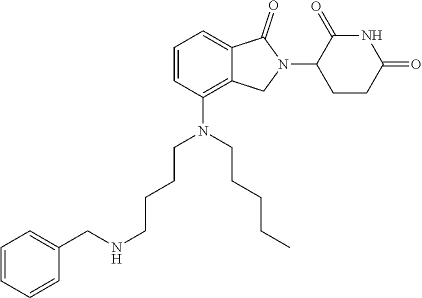 [SMILES: CCCCCN(CCCCNCc1ccccc1)c1cccc2c1CN(C1CCC(=O)NC1=O)C2=O] | 3-(4-((4- (benzylamino)butyl)(pentyl)amino)-1- oxoisoindolin-2-yl)piperidine-2,6-dione |
| 164 | 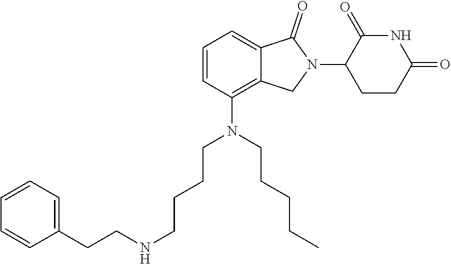 [SMILES: CCCCCN(CCCCNCCc1ccccc1)c1cccc2c1CN(C1CCC(=O)NC1=O)C2=O] | 3-(1-oxo-4-(pentyl(4- (phenethylamino)butyl)amino)isoindolin- 2-yl)piperidine-2,6-dione |
| 165 | 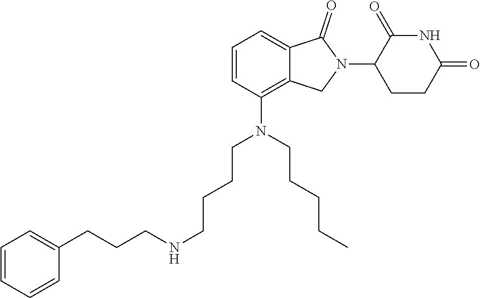 [SMILES: CCCCCN(CCCCNCCCc1ccccc1)c1cccc2c1CN(C1CCC(=O)NC1=O)C2=O] | 3-(1-oxo-4-(pentyl(4-((3- phenylpropyl)amino)butyl)amino) isoindolin-2-yl)piperidine-2,6-dione |
| 166 | 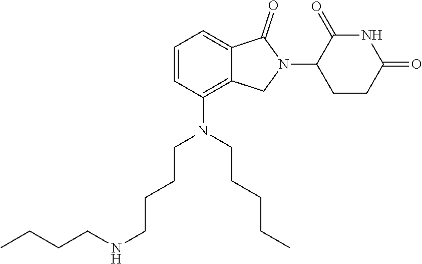 [SMILES: CCCCCN(CCCCNCCCC)c1cccc2c1CN(C1CCC(=O)NC1=O)C2=O] | 3-(4-((4- (butylamino)butyl)(pentyl)amino)-1- oxoisoindolin-2-yl)yl)piperidine-2,6-dione |
| 167 | 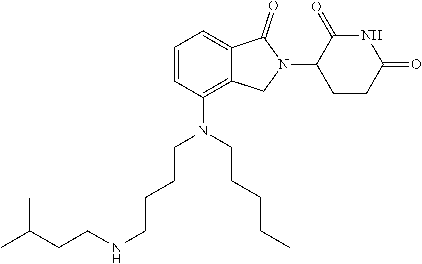 [SMILES: CCCCCN(CCCCNCCC(C)C)c1cccc2c1CN(C1CCC(=O)NC1=O)C2=O] | 3-(4-((4- (isopentylamino)butyl)(pentyl)amino)- 1-oxoisoindolin-2-yl)yl)piperidine-2,6-dione |
| 168 | 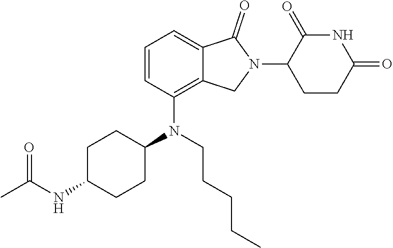 [SMILES: CCCCCN(c1cccc2c1CN(C1CCC(=O)NC1=O)C2=O)[C@H]1CC[C@H](NC(C)=O)CC1] | N-((1,4r)-4-((2-(2,6-dioxopiperidin-3- yl)-1-oxoisoindolin-4- yl)(pentyl)amino)cyclohexyl)acetamide |
| 169 | 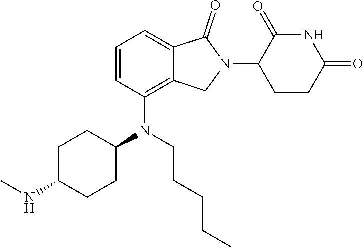 [SMILES: CCCCCN(c1cccc2c1CN(C1CCC(=O)NC1=O)C2=O)[C@H]1CC[C@H](NC)CC1] | 3-(4-(((1r,4r)-4- (methylamino)cyclohexyl)(pentyl)amino)- 1-oxoisoindolin-2-yl)yl)piperidine-2,6-dione |
| 181 | 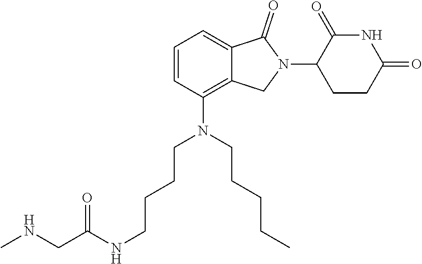 [SMILES: CCCCCN(CCCCNC(=O)CNC)c1cccc2c1CN(C1CCC(=O)NC1=O)C2=O] | N-(4-((2-(2,6-dioxopiperidin-3-yl)-1- oxoisoindolin-4- yl(pentyl)amino)butyl)-2- (methylamino)acetamide |
| 223 | 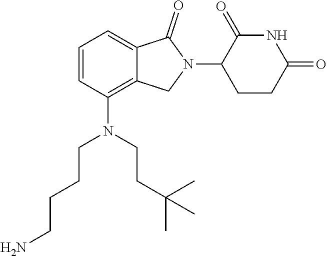 [SMILES: CC(C)(C)CCN(CCCCN)c1cccc2c1CN(C1CCC(=O)NC1=O)C2=O] | 3-(4-((4-aminobutyl)(3,3- dimethylbutyl)amino)-1-oxoisoindolin- 2-yl)piperidine-2,6-dione |
| 235 | 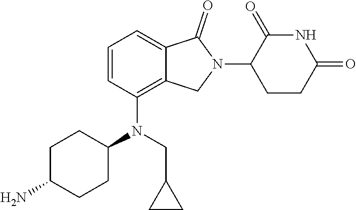 [SMILES: N[C@H]1CC[C@H](N(CC2CC2)c2cccc3c2CN(C2CCC(=O)NC2=O)C3=O)CC1] | 3-(4-(((1r,4r)-4- aminocyclohexyl)(cyclopropylmethyl) amino)-1-oxoisoindolin-2- yl)piperidine-2,6-dione |
| 236 | 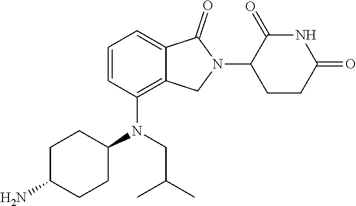 [SMILES: CC(C)CN(c1cccc2c1CN(C1CCC(=O)NC1=O)C2=O)[C@H]1CC[C@H](N)CC1] | 3-(4-(((1,4r)-4- aminocyclohexyl)(isobutyl)amino)-1- oxoisoindolin-2-yl)piperidine-2,6- dione |
| 237 | 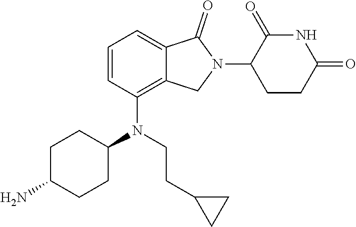 [SMILES: N[C@H]1CC[C@H](N(CCC2CC2)c2cccc3c2CN(C2CCC(=O)NC2=O)C3=O)CC1] | 3-(4-(((1r,4r)-4-aminocyclohexyl)(2- cyclopropylethyl)amino)-1- oxoisoindolin-2-yl)piperidine-2,6-dione |
| 238 | 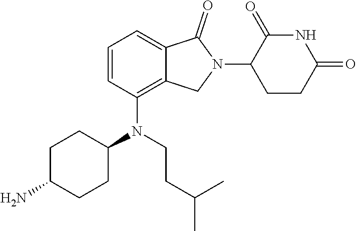 [SMILES: CC(C)CCN(c1cccc2c1CN(C1CCC(=O)NC1=O)C2=O)[C@H]1CC[C@H](N)CC1] | 3-(4-(((1r,4r)-4- aminocyclohexyl)(isopentyl)amino)-1- oxoisoindolin-2-yl)piperidine-2,6-dione |
| 239 | 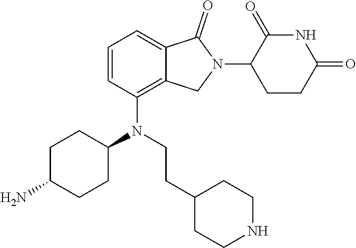 [SMILES: N[C@H]1CC[C@H](N(CCC2CCNCC2)c2cccc3c2CN(C2CCC(=O)NC2=O)C3=O)CC1] | 3-(4-(((1r,4r)-4-aminocyclohexyl)(2- (piperidin-4-yl)ethyl)amino)-1- oxoisoindolin-2-yl)yl)piperidine-2,6-dione |
| 240 | 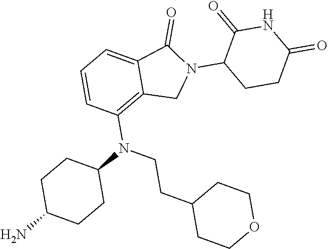 [SMILES: N[C@H]1CC[C@H](N(CCC2CCOCC2)c2cccc3c2CN(C2CCC(=O)NC2=O)C3=O)CC1] | 3-(4-(((1r,4r)-4-aminocyclohexyl)(2- (tetrahydro-2H-pyran-4- yl)ethyl)amino)-1-oxoisoindolin-2- yl)piperidine-2,6-dione |
| 241 | 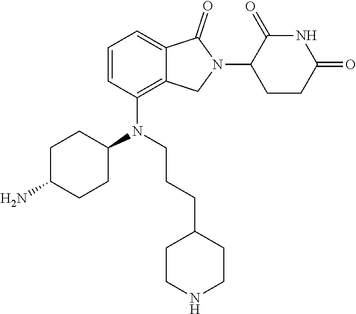 [SMILES: N[C@H]1CC[C@H](N(CCCC2CCNCC2)c2cccc3c2CN(C2CCC(=O)NC2=O)C3=O)CC1] | 3-(4-(((1r,4r)-4-aminocyclohexyl)(3- (piperidin-4-yl)propyl)amino)-1- oxoisoindolin-2-yl)yl)piperidine-2,6-dione |
| 242 | 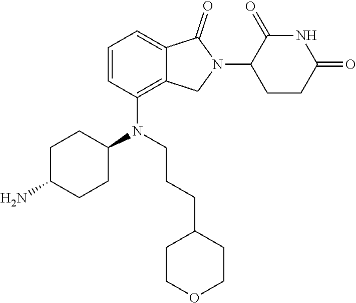 [SMILES: N[C@H]1CC[C@H](N(CCCC2CCOCC2)c2cccc3c2CN(C2CCC(=O)NC2=O)C3=O)CC1] | 3-(4-(((1r,4r)-4-aminocyclohexyl)(3- (tetrahydro-2H-pyran-4- yl)propyl)amino)-1-oxoisoindolin-2- yl)yl)piperidine-2,6-dione |
| 243 | 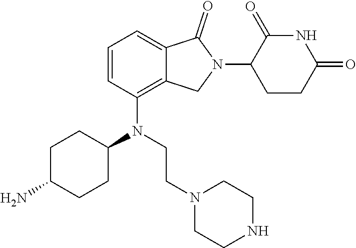 [SMILES: N[C@H]1CC[C@H](N(CCN2CCNCC2)c2cccc3c2CN(C2CCC(=O)NC2=O)C3=O)CC1] | 3-(4-(((1r,4r)-4-aminocyclohexyl)(2- (piperazin-1-yl)ethyl)amino)-1- oxoisoindolin-2-yl)yl)piperidine-2,6-dione |
| 244 | 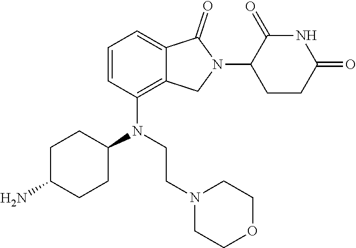 [SMILES: N[C@H]1CC[C@H](N(CCN2CCOCC2)c2cccc3c2CN(C2CCC(=O)NC2=O)C3=O)CC1] | 3-(4-(((1r,4r)-4-aminocyclohexyl)(2- morpholinoethyl)amino)-1- oxoisoindolin-2-yl)yl)piperidine-2,6-dione |
| 245 | 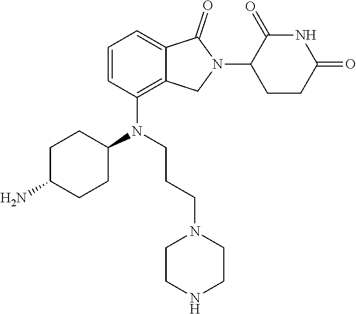 [SMILES: N[C@H]1CC[C@H](N(CCCN2CCNCC2)c2cccc3c2CN(C2CCC(=O)NC2=O)C3=O)CC1] | 3-(4-(((1r,4r)-4-aminocyclohexyl)(3- (piperazin-1-yl)propyl)amino)-1- oxoisoindolin-2-yl)piperidine-2,6-dione |
| 246 | 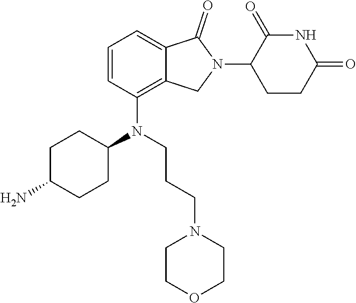 [SMILES: N[C@H]1CC[C@H](N(CCCN2CCOCC2)c2cccc3c2CN(C2CCC(=O)NC2=O)C3=O)CC1] | 3-(4-(((1r,4r)-4-aminocyclohexyl)(3- morpholinopropyl)amino)-1- oxoisoindolin-2-yl)piperidine-2,6-dione |
| 247 | 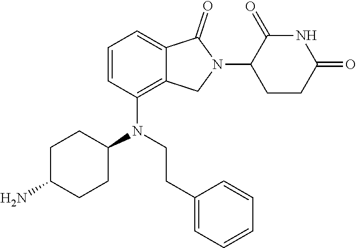 [SMILES: N[C@H]1CC[C@H](N(CCc2ccccc2)c2cccc3c2CN(C2CCC(=O)NC2=O)C3=O)CC1] | 3-(4-(((1r,4r)-4- aminocyclohexyl)(phenethyl)amino)-1- oxoisoindolin-2-yl)piperidine-2,6- dione |
| 251 | 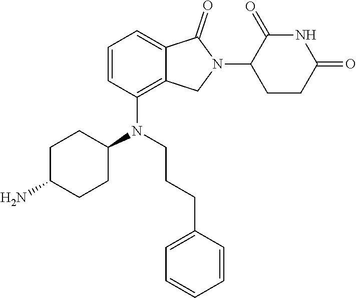 [SMILES: N[C@H]1CC[C@H](N(CCCc2ccccc2)c2cccc3c2CN(C2CCC(=O)NC2=O)C3=O)CC1] | 3-(4-(((1r,4r)-4-aminocyclohexyl)(3- phenylpropyl)amino)-1-oxoisoindolin- 2-yl)piperidine-2,6-dione |
| 255 | 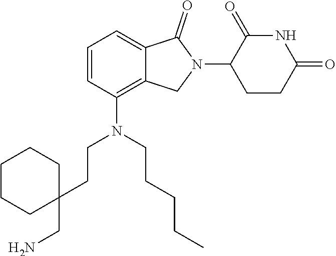 [SMILES: CCCCCN(CCC1(CN)CCCCC1)c1cccc2c1CN(C1CCC(=O)NC1=O)C2=O] | 3-(4-((2-(1- (aminomethyl)cyclohxyl)ethyl)(pentyl) amino)-1-oxoisoindolin-2- yl)piperidine-2,6-dione |
| 267 | 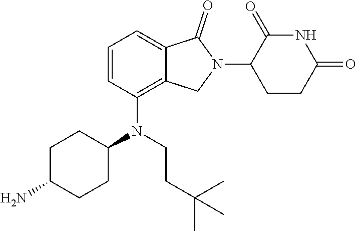 [SMILES: CC(C)(C)CCN(c1cccc2c1CN(C1CCC(=O)NC1=O)C2=O)[C@H]1CC[C@H](N)CC1] | 3-(4-(((1r,4r)-4-aminocyclohexyl)(3,3- dimethylbutyl)amino)-1-oxoisoindolin- 2-yl)piperidine-2,6-dione |
| 268 | 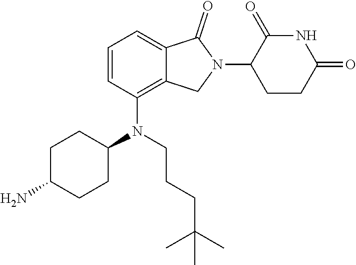 [SMILES: CC(C)(C)CCCN(c1cccc2c1CN(C1CCC(=O)NC1=O)C2=O)[C@H]1CC[C@H](N)CC1] | 3-(4-(((1r,4r)-4-aminocyclohexyl)(4,4- dimethylpentyl)amino)-1- oxoisoindolin-2-yl)piperidine-2,6-dione |
| 273 | 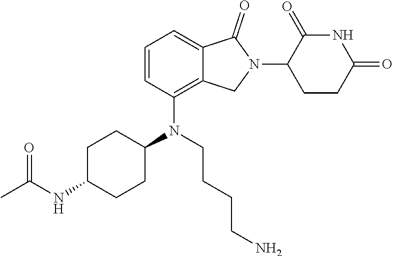 [SMILES: CC(=O)N[C@H]1CC[C@H](N(CCCCN)c2cccc3c2CN(C2CCC(=O)NC2=O)C3=O)CC1] | N-((1r,4r)-4-((4-aminobutyl)(2-(2,6- dioxopiperidin-3-yl)-1-oxoisoindolin- 4-yl)amino)cyclohexyl)acetamide |
| 305 | 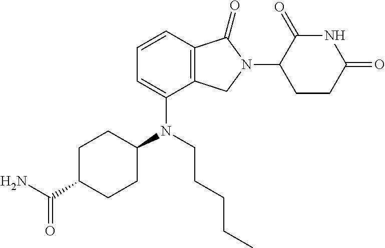 [SMILES: CCCCCN(c1cccc2c1CN(C1CCC(=O)NC1=O)C2=O)[C@H]1CC[C@H](C(N)=O)CC1] | (1r,4r)-4-((2-(2,6-dioxopiperidin-3-yl)- 1-oxoisoindolin-4- yl)(pentyl)amino)cyclohexanecarboxamide |
| 306 | 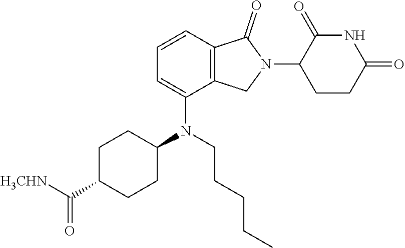 | (1r,4r)-4-((2-(2,6-dioxopiperidin-3-yl)- 1-oxoisoindolin-4-yl)(pentyl)amino)- N-methylcyclohexanecarboxamide |
| 307 | 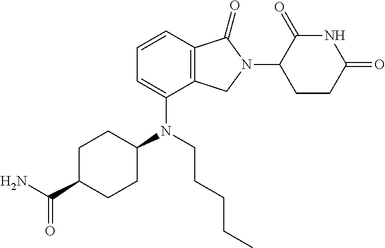 [SMILES: CCCCCN(c1cccc2c1CN(C1CCC(=O)NC1=O)C2=O)[C@H]1CC[C@@H](C(N)=O)CC1] | (1s,4s)-4-((2-(2,6-dioxopiperidin-3-yl)- 1-oxoisoindolin-4- yl)(pentyl)amino)cyclohexanecarboxamide |
| 308 | 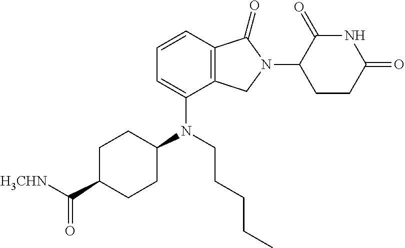 | (1s,4s)-4-((2-(2,6-dioxopiperidin-3-yl)- 1-oxoisoindolin-4-yl)(pentyl)amino)- N-methylcyclohexanecarboxamide |
| 309 | 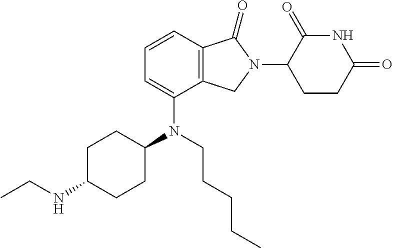 [SMILES: CCCCCN(c1cccc2c1CN(C1CCC(=O)NC1=O)C2=O)[C@H]1CC[C@H](NCC)CC1] | 3-(4-(((1r,4r)-4- (ethylamino)cyclohexyl)(pentyl)amino)- 1-oxoisoindolin-2-yl)piperidine-2,6-dione |
| 310 | 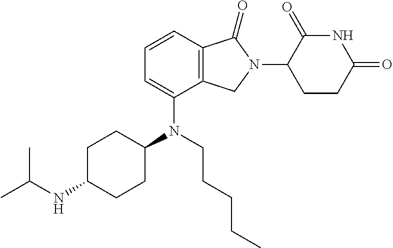 [SMILES: CCCCCN(c1cccc2c1CN(C1CCC(=O)NC1=O)C2=O)[C@H]1CC[C@H](NC(C)C)CC1] | 3-(4-(((1r,4r)-4- (isopropylamino)cyclohexyl)(pentyl) amino)-1-oxoisoindolin-2-yl)piperidine- 2,6-dione |
| 311 | 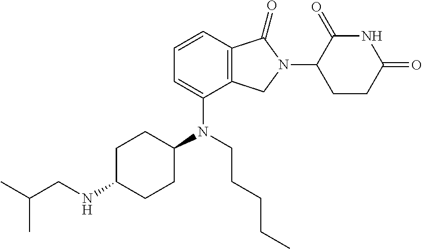 [SMILES: CCCCCN(c1cccc2c1CN(C1CCC(=O)NC1=O)C2=O)[C@H]1CC[C@H](NCC(C)C)CC1] | 3-(4-(((1r,4r)-4- (isobutylamino)cyclohexyl)(pentyl) amino)-1-oxoisoindolin-2-yl)piperidine- 2,6-dione |
| 312 | 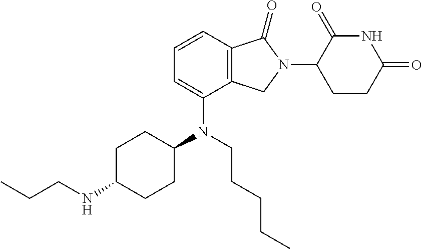 [SMILES: CCCCCN(c1cccc2c1CN(C1CCC(=O)NC1=O)C2=O)[C@H]1CC[C@H](NCCC)CC1] | 3-(1-oxo-4-(pentyl((1r,4r)-4- (propylamino)cyclohexyl)amino) isoindolin-2-yl)piperidine-2,6-dione |
| 313 | 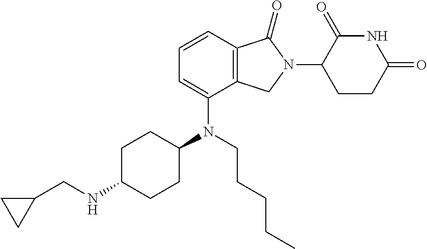 [SMILES: CCCCCN(c1cccc2c1CN(C1CCC(=O)NC1=O)C2=O)[C@H]1CC[C@H](NCC2CC2)CC1] | 3-(4-(((1r,4r)-4- ((cyclopropylmethyl)amino)cyclohexyl) (pentyl)amino)-1-oxoisoindolin-2- yl)piperidine-2,6-dione |
| 314 | 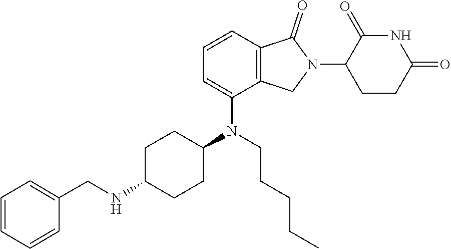 [SMILES: CCCCCN(c1cccc2c1CN(C1CCC(=O)NC1=O)C2=O)[C@H]1CC[C@H](NCc2ccccc2)CC1] | 3-(4-(((1r,4r)-4- (benzylamino)cyclohexyl)(pentyl) amino)-1-oxoisoindolin-2-yl)piperidine- 2,6-dione |
| 323 | 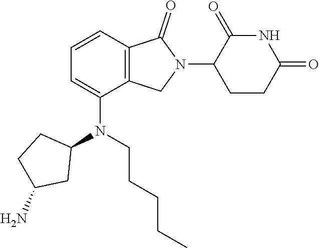 [SMILES: CCCCCN(c1cccc2c1CN(C1CCC(=O)NC1=O)C2=O)[C@@H]1CC[C@@H](N)C1] | 3-(4-(((1R,3R)-3- aminocyclopentyl)(pentyl)amino)-1- oxoisoindolin-2-yl)piperidine-2,6-dione |
| 324 | 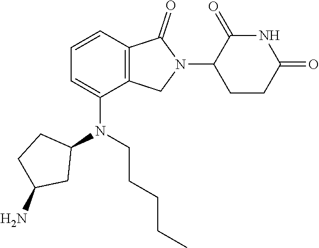 [SMILES: CCCCCN(c1cccc2c1CN(C1CCC(=O)NC1=O)C2=O)[C@@H]1CC[C@H](N)C1] | 3-(4-(((1R,3S)-3- aminocyclopentyl)(pentyl)amino)-1- oxoisoindolin-2-yl)piperidine-2,6-dione |
| 325 | 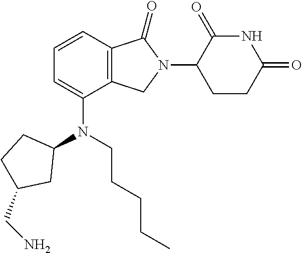 [SMILES: CCCCCN(c1cccc2c1CN(C1CCC(=O)NC1=O)C2=O)[C@@H]1CC[C@@H](CN)C1] | 3-(4-(((1R,3R)-3- (aminomethyl)cyclopentyl)(pentyl) amino)-1-oxoisoindolin-2-yl)piperidine- 2,6-dione |
| 325A | 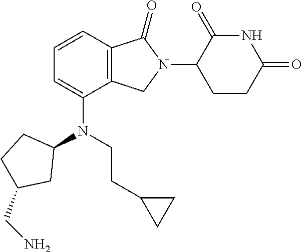 [SMILES: NC[C@@H]1CC[C@@H](N(CCC2CC2)c2cccc3c2CN(C2CCC(=O)NC2=O)C3=O)C1] | 3-(4-(((1R,3R)-3- (aminomethyl)cyclopentyl)(2- cyclopropylethyl)amino)-1- oxoisoindolin-2-yl)piperidine-2,6-dione |
| 326 | 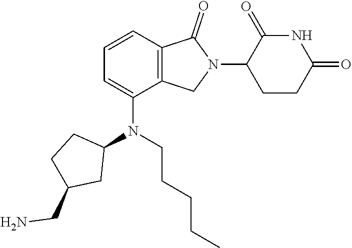 [SMILES: CCCCCN(c1cccc2c1CN(C1CCC(=O)NC1=O)C2=O)[C@@H]1CC[C@H](CN)C1] | 3-(4-(((1R,3S)-3- (aminomethyl)cyclopentyl)(pentyl) amino)-1-oxoisoindolin-2-yl)piperidine- 2,6-dione |
| 326A | 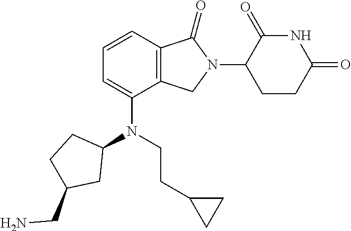 [SMILES: NC[C@H]1CC[C@@H](N(CCC2CC2)c2cccc3c2CN(C2CCC(=O)NC2=O)C3=O)C1] | 3-(4-(((1R,3S)-3- (aminomethyl)cyclopentyl)(2- cyclopropylethyl)amino)-1- oxoisoindolin-2-yl)piperidine-2,6-dione |
| 327 | 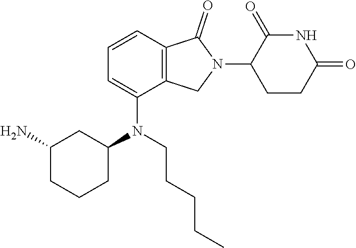 [SMILES: CCCCCN(c1cccc2c1CN(C1CCC(=O)NC1=O)C2=O)[C@H]1CCC[C@H](N)C1] | 3-(4-(((1S,3S)-3- aminocyclohexyl)(pentyl)amino)-1- oxoisoindolin-2-yl)piperidine-2,6-dione |
| 328 | 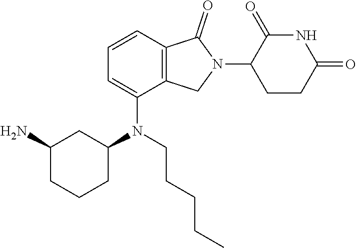 [SMILES: CCCCCN(c1cccc2c1CN(C1CCC(=O)NC1=O)C2=O)[C@H]1CCC[C@@H](N)C1] | 3-(4-(((1S,3R)-3- aminocyclohexyl)(pentyl)amino)-1- oxoisoindolin-2-yl)piperidine-2,6-dione |
| 329 | 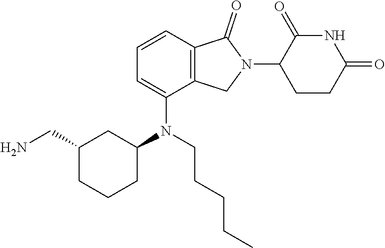 [SMILES: CCCCCN(c1cccc2c1CN(C1CCC(=O)NC1=O)C2=O)[C@H]1CCC[C@H](CN)C1] | 3-(4-(((1S,3S)-3- (aminomethyl)cyclohexyl)(pentyl)amino)- 1-oxoisoindolin-2-yl)piperidine-2,6-dione |
| 330 | 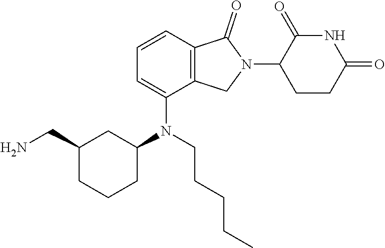 [SMILES: CCCCCN(c1cccc2c1CN(C1CCC(=O)NC1=O)C2=O)[C@H]1CCC[C@@H](CN)C1] | 3-(4-(((1S,3R)-3- (aminomethyl)cyclohexyl)(pentyl) amino)-1-oxoisoindolin-2-yl)piperidine- 2,6-dione |
| 331 | 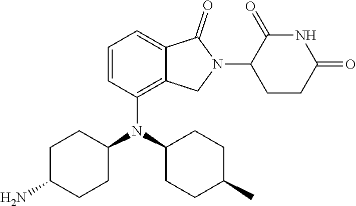 [SMILES: C[C@H]1CC[C@@H](N(c2cccc3c2CN(C2CCC(=O)NC2=O)C3=O)[C@H]2CC[C@H](N)CC2)CC1] | 3-(4-(((1r,4R)-4- aminocyclohexyl)((1s,4S)-4- methylcyclohexyl)amino)-1- oxoisoindolin-2-yl)piperidine-2,6-dione |
| 332 | 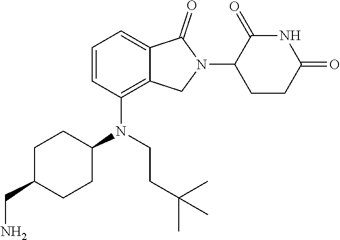 [SMILES: CC(C)(C)CCN(c1cccc2c1CN(C1CCC(=O)NC1=O)C2=O)[C@H]1CC[C@@H](CN)CC1] | 3-(4-(((1s,4s)-4- (aminomethyl)cyclohexyl)(3,3- dimethylbutyl)amino)-1-oxoisoindolin- 2-yl)piperidine-2,6-dione |
| 333 | 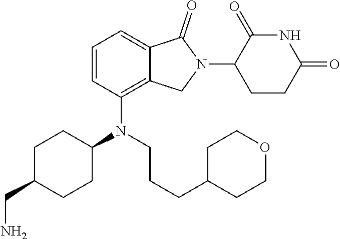 [SMILES: NC[C@H]1CC[C@@H](N(CCCC2CCOCC2)c2cccc3c2CN(C2CCC(=O)NC2=O)C3=O)CC1] | 3-(4-(((1s,4s)-4- (aminomethyl)cyclohexyl)(3- (tetrahydro-2H-pyran-4- yl)propyl)amino)-1-oxoisoindolin-2- yl)piperidine-2,6-dione |
| 334 | 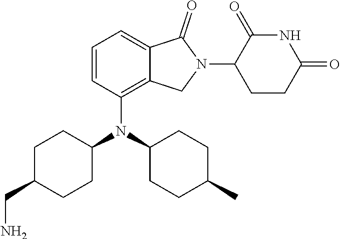 [SMILES: C[C@H]1CC[C@@H](N(c2cccc3c2CN(C2CCC(=O)NC2=O)C3=O)[C@H]2CC[C@@H](CN)CC2)CC1] | 3-(4-(((1s,4S)-4- (aminomethyl)cyclohexyl)((1s,4S)-4- methylcyclohexyl)amino)-1- oxoisoindolin-2-yl)piperidine-2,6-dione |
| 340 | 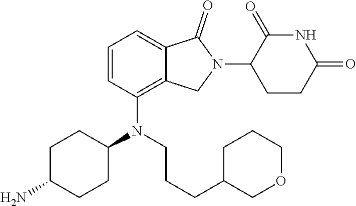 [SMILES: N[C@H]1CC[C@H](N(CCCC2CCCOC2)c2cccc3c2CN(C2CCC(=O)NC2=O)C3=O)CC1] | 3-(4-(((1r,4r)-4-aminocyclohexyl)(3- (tetrahydro-2H-pyran-3- yl)propyl)amino)-1-oxoisoindolin-2- yl)piperidine-2,6-dione |
| 341 | 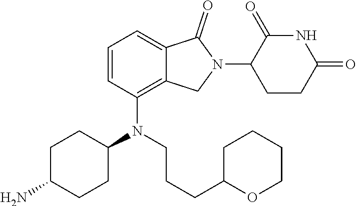 [SMILES: N[C@H]1CC[C@H](N(CCCC2CCCCO2)c2cccc3c2CN(C2CCC(=O)NC2=O)C3=O)CC1] | 3-(4-(((1r,4r)-4-aminocyclohexyl)(3- (tetrahydro-2H-pyran-2- yl)propyl)amino)-1-oxoisoindolin-2- yl)piperidine-2,6-dione |
| 344 | 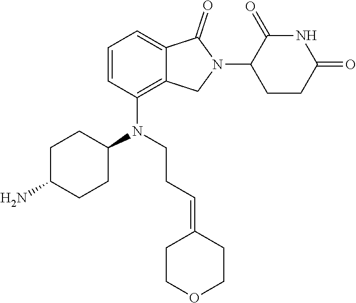 [SMILES: N[C@H]1CC[C@H](N(CCC=C2CCOCC2)c2cccc3c2CN(C2CCC(=O)NC2=O)C3=O)CC1] | 3-(4-(((1r,4r)-4-aminocyclohexyl)(3- (dihydro-2H-pyran-4(3H)- ylidene)propyl)amino)-1- oxoisoindolin-2-yl)yl)piperidine-2,6-dione |
| 345 | 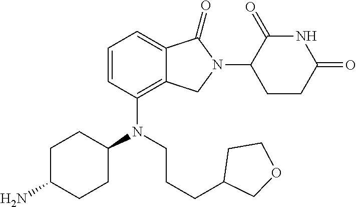 [SMILES: N[C@H]1CC[C@H](N(CCCC2CCOC2)c2cccc3c2CN(C2CCC(=O)NC2=O)C3=O)CC1] | 3-(4-(((1r,4r)-4-aminocyclohexyl)(3- (tetrahydro-3-yl)propyl)amino)-1- oxoisoindolin-2-yl)piperidine-2,6-dione |
| 346 | 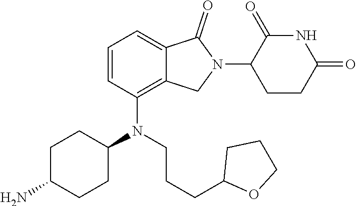 [SMILES: N[C@H]1CC[C@H](N(CCCC2CCCO2)c2cccc3c2CN(C2CCC(=O)NC2=O)C3=O)CC1] | 3-(4-(((1r,4r)-4-aminocyclohexyl)(3- (tetrahydrofuran-2-yl)propyl)amino)-1- oxoisoindolin-2-yl)piperidine-2,6-dione |
| 347 | 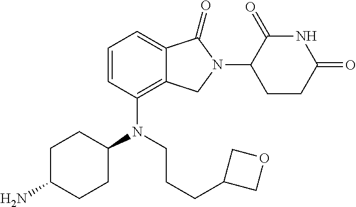 [SMILES: N[C@H]1CC[C@H](N(CCCC2COC2)c2cccc3c2CN(C2CCC(=O)NC2=O)C3=O)CC1] | 3-(4-(((1r,4r)-4-aminocyclohexyl)(3- (oxetan-3-y)propyl)amino)-1- oxoisoindolin-2-yl)piperidine-2,6-dione |
| 349 | 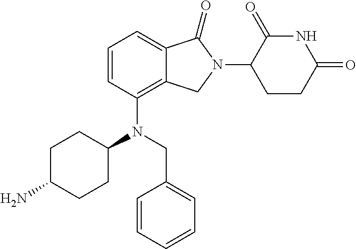 [SMILES: N[C@H]1CC[C@H](N(Cc2ccccc2)c2cccc3c2CN(C2CCC(=O)NC2=O)C3=O)CC1] | 3-(4-(((1r,4r)-4- aminocyclohexyl)(benzyl)amino)-1- oxoisoindolin-2-yl)piperidine-2,6-dione |
| 352 | 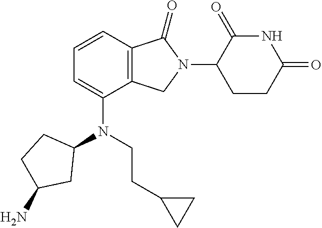 [SMILES: N[C@H]1CC[C@@H](N(CCC2CC2)c2cccc3c2CN(C2CCC(=O)NC2=O)C3=O)C1] | 3-(4-(((1R,3S)-3-aminocyclopentyl)(2- cyclopropylethyl)amino)-1- oxoisoindolin-2-yl)piperidine-2,6-dione |
| 353 | 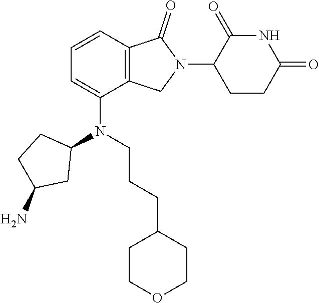 [SMILES: N[C@H]1CC[C@@H](N(CCCC2CCOCC2)c2cccc3c2CN(C2CCC(=O)NC2=O)C3=O)C1] | 3-(4-(((1R,3S)-3-aminocyclopentyl)(3- (tetrahydro-2H-pyran-4- yl)propyl)amino)-1-oxoisoindolin-2- yl)piperidine-2,6-dione |
| 354 | 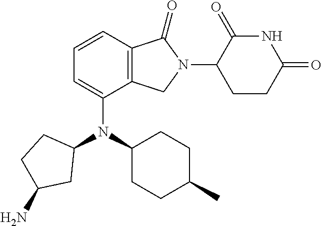 [SMILES: C[C@H]1CC[C@@H](N(c2cccc3c2CN(C2CCC(=O)NC2=O)C3=O)[C@@H]2CC[C@H](N)C2)CC1] | 3-(4-(((1R,3S)-3- aminocyclopentyl)((1s,4S)-4- methylcyclohexyl)amino)-1- oxoisoindolin-2-yl)piperidine-2,6-dione |
| 355 | 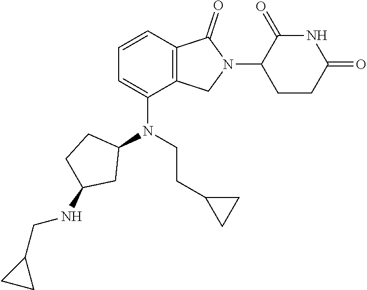 [SMILES: O=C1CCC(N2Cc3c(cccc3N(CCC3CC3)[C@@H]3CC[C@H](NCC4CC4)C3)C2=O)C(=O)N1] | 3-(4-((2-cyclopropylethyl)((1R,3S)-3- ((cyclopropylmethyl)amino)cyclopentyl)- amino)-1-oxoisoindolin-2- yl)piperidine-2,6-dione |
| 356 | 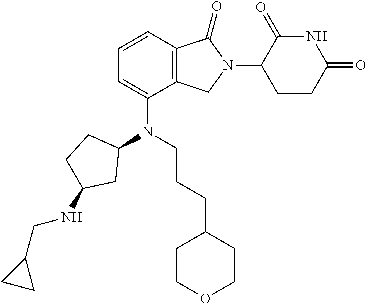 [SMILES: O=C1CCC(N2Cc3c(cccc3N(CCCC3CCOCC3)[C@@H]3CC[C@H](NCC4CC4)C3)C2=O)C(=O)N1] | 3-(4-(((1R,3S)-3- ((cyclopropylmethyl)amino)cyclopentyl) (3-(tetrahydro-2H-pyran-4- yl)propyl)amino)-1-oxoisoindolin-2- yl)piperidine-2,6-dione |
| 358 | 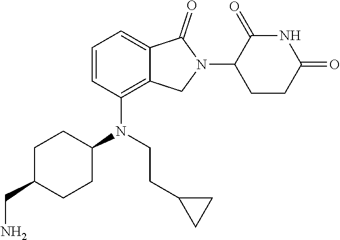 [SMILES: NC[C@H]1CC[C@@H](N(CCC2CC2)c2cccc3c2CN(C2CCC(=O)NC2=O)C3=O)CC1] | 3-(4-(((1s,4s)-4- (aminomethyl)cyclohexyl)(2- cyclopropylethyl)amino)-1- oxoisoindolin-2-yl)piperidine-2,6-dione |
| 359 | 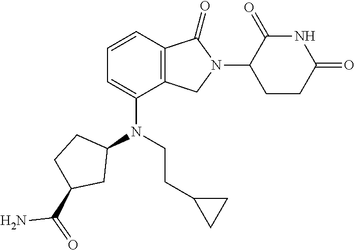 [SMILES: NC(=O)[C@H]1CC[C@@H](N(CCC2CC2)c2cccc3c2CN(C2CCC(=O)NC2=O)C3=O)C1] | (1S,3R)-3-((2-cyclopropylethyl)(2- (2,6-dioxopiperidin-3-yl)-1- oxoisoindolin-4- yl)amino)cyclopentanecarboxamide |
| 360 | 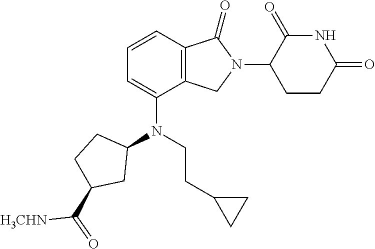 | (1S,3R)-3-((2-cyclopropylethyl)(2- (2,6-dioxopiperidin-3-yl)-1- oxoisoindolin-4-yl)amino)-N- methylcyclopentanecarboxamide |
| 361 | 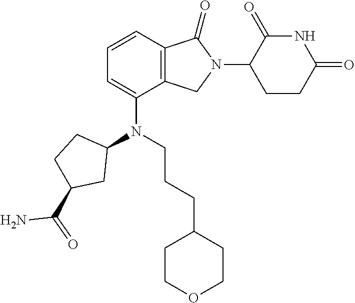 [SMILES: NC(=O)[C@H]1CC[C@@H](N(CCCC2CCOCC2)c2cccc3c2CN(C2CCC(=O)NC2=O)C3=O)C1] | (1S,3R)-3-((2-(2,6-dioxopiperidin-3- yl)-1-oxoisoindolin-4-yl)(3- (tetrahydro-2H-pyran-4- yl)propyl)amino)cyclopentanecarboxamide |
| 362 | 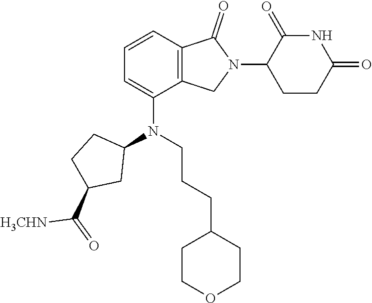 | (1S,3R)-3-((2-(2,6-dioxopiperidin-3- yl)-1-oxoisoindolin-4-yl)(3- (tetrahydro-2H-pyran-4- yl)propyl)amino)-N- methylcyclopentanecarboxamide |
| 512 | 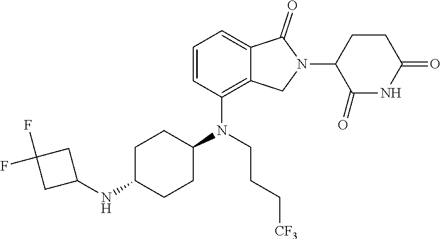 [SMILES: O=C1CCC(N2Cc3c(cccc3N(CCCC(F)(F)F)[C@H]3CC[C@H](NC4CC(F)(F)C4)CC3)C2=O)C(=O)N1] | 3-(4-(((1r,4r)-4-((3,3- difluorocyclobutyl)amino)cyclohexyl) (4,4,4-trifluorobutyl)amino)-1- oxoisoindolin-2-yl)piperidine-2,6-dione |
| 513 | 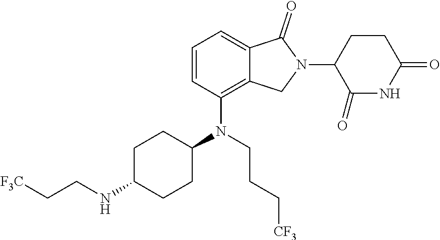 [SMILES: O=C1CCC(N2Cc3c(cccc3N(CCCC(F)(F)F)[C@H]3CC[C@H](NCCC(F)(F)F)CC3)C2=O)C(=O)N1] | 3-(1-oxo-4-((4,4,4- trifluorobutyl)((1r,4r)-4-((3,3,3- trifluoropropyl)amino)cyclohexyl)amino) isoindolin-2-yl)piperidine-2,6-dione |
| 514 | 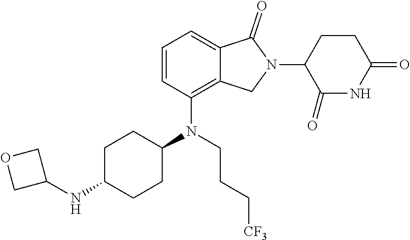 [SMILES: O=C1CCC(N2Cc3c(cccc3N(CCCC(F)(F)F)[C@H]3CC[C@H](NC4COC4)CC3)C2=O)C(=O)N1] | 3-(4-(((1r,4r)-4-(oxetan-3- ylamino)cyclohexyl)(4,4,4- trifluorobutyl)amino)-1-oxoisoindolin- 2-yl)piperidine-2,6-dione |
| 515 | 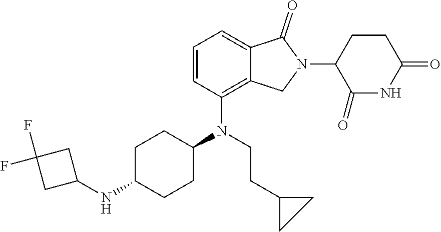 [SMILES: O=C1CCC(N2Cc3c(cccc3N(CCC3CC3)[C@H]3CC[C@H](NC4CC(F)(F)C4)CC3)C2=O)C(=O)N1] | 3-(4-((2-cyclopropylethyl)((1r,4r)-4- ((3,3- difluorocyclobutyl)amino)cyclohexyl) amino)-1-oxoisoindolin-2-yl)piperidine- 2,6-dione |
| 515A | 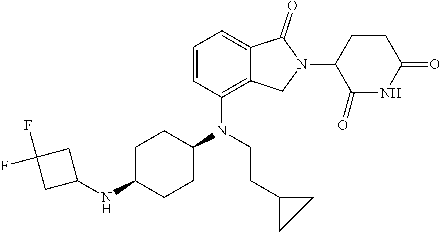 [SMILES: O=C1CCC(N2Cc3c(cccc3N(CCC3CC3)[C@H]3CC[C@@H](NC4CC(F)(F)C4)CC3)C2=O)C(=O)N1] | 3-(4-((2-cyclopropylethyl)((1s,4s)-4- ((3,3- difluorocyclobutyl)amino)cyclohexyl) amino)-1-oxoisoindolin-2-yl)piperidine- 2,6-dione |
| 516 | 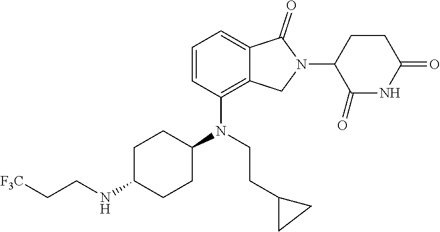 [SMILES: O=C1CCC(N2Cc3c(cccc3N(CCC3CC3)[C@H]3CC[C@H](NCCC(F)(F)F)CC3)C2=O)C(=O)N1] | 3-(4-((2-cyclopropylethyl)((1r,4r)-4- ((3,3,3- trifluoropropyl)amino)cyclohexyl) amino)-1-oxoisoindolin-2-yl)piperidine- 2,6-dione |
| 517 | 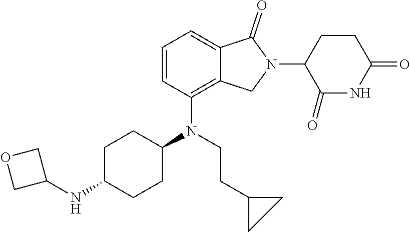 [SMILES: O=C1CCC(N2Cc3c(cccc3N(CCC3CC3)[C@H]3CC[C@H](NC4COC4)CC3)C2=O)C(=O)N1] | 3-(4-((2-cyclopropylethyl)((1r,4r)-4- (oxetan-3-ylamino)cyclohexyl)amino)- 1-oxoisoindolin-2-yl)piperidine-2,6-dione |
| 518 | 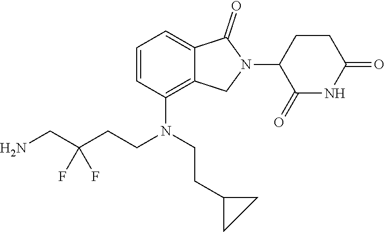 [SMILES: NCC(F)(F)CCN(CCC1CC1)c1cccc2c1CN(C1CCC(=O)NC1=O)C2=O] | 3-(4-((4-amino-3,3-difluorobutyl)(2- cyclopropylethyl)amino)-1- oxoisoindolin-2-yl)piperidine-2,6- dione |
| 519 | 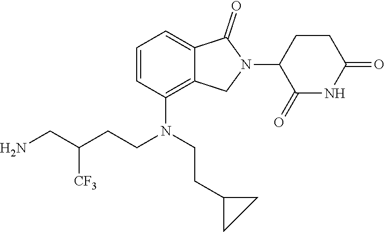 [SMILES: NCC(CCN(CCC1CC1)c1cccc2c1CN(C1CCC(=O)NC1=O)C2=O)C(F)(F)F] | 3-(4-((3-(aminomethyl)-4,4,4- trifluorobutyl)(2- cyclopropylethyl)amino)-1- oxoisoindolin-2-yl)piperidine-2,6-dione |
| 521 | 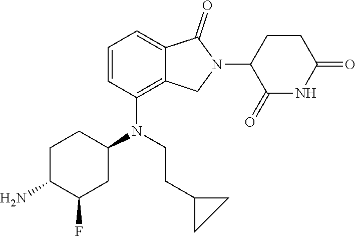 [SMILES: N[C@@H]1CC[C@@H](N(CCC2CC2)c2cccc3c2CN(C2CCC(=O)NC2=O)C3=O)C[C@H]1F] | 3-(4-(((1R,3R,4R)-4-amino-3- fluorocyclohexyl)(2- cyclopropylethyl)amino)-1- oxoisoindolin-2-yl)piperidine-2,6-dione |
| 522 | 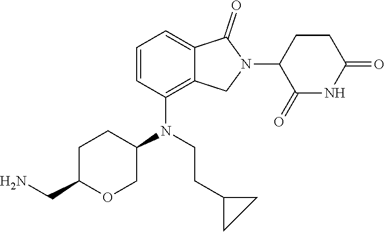 [SMILES: NC[C@H]1CC[C@@H](N(CCC2CC2)c2cccc3c2CN(C2CCC(=O)NC2=O)C3=O)CO1] | 3-(4-(((3R,6R)-6- (aminomethyl)tetrahydro-2H-pyran-3- yl)(2-cyclopropylethyl)amino)-1- oxoisoindolin-2-yl)piperidine-2,6-dione |
| 523 | 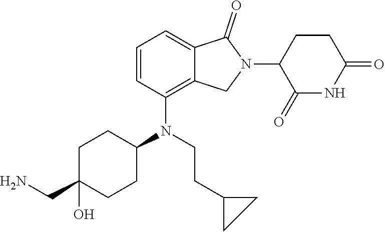 [SMILES: NC[C@]1(O)CC[C@H](N(CCC2CC2)c2cccc3c2CN(C2CCC(=O)NC2=O)C3=O)CC1] | 3-(4-(((1r,4r)-4-(aminomethyl)-4- hydroxycyclohexyl)(2- cyclopropylethyl)amino)-1- oxoisoindolin-2-yl)piperidine-2,6-dione |
| 530 | 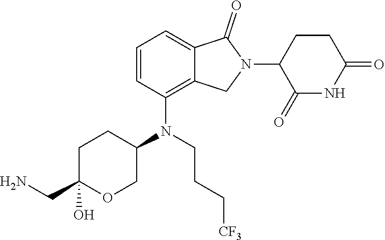 [SMILES: NC[C@@]1(O)CC[C@@H](N(CCCC(F)(F)F)c2cccc3c2CN(C2CCC(=O)NC2=O)C3=O)CO1] | 3-(4-(((1r,4r)-4-(aminomethyl)-4- hydroxycyclohexyl)(4,4,4- trifluorobutyl)amino)-1-oxoisoindolin- 2-yl)piperidine-2,6-dione |
| 532 | 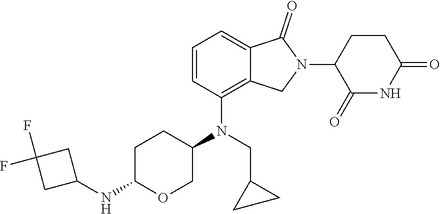 [SMILES: O=C1CCC(N2Cc3c(cccc3N(CC3CC3)[C@@H]3CC[C@@H](NC4CC(F)(F)C4)OC3)C2=O)C(=O)N1] | 3-(4-((cyclopropylmethyl)((1r,4r)-4- ((3,3- difluorocyclobutyl)amino)cyclohexyl) amino)-1-oxoisoindolin-2-yl)piperidine- 2,6-dione |
| 533 | 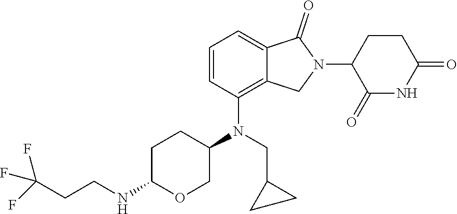 [SMILES: O=C1CCC(N2Cc3c(cccc3N(CC3CC3)[C@@H]3CC[C@@H](NCCC(F)(F)F)OC3)C2=O)C(=O)N1] | 3-(4-((cyclopropylmethyl)((1r,4r)-4- ((3,3,3- trifluoropropyl)amino)cyclohexyl) amino)-1-oxoisoindolin-2-yl)piperidine- 2,6-dione |
| 534 | 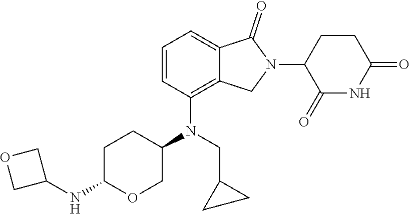 [SMILES: O=C1CCC(N2Cc3c(cccc3N(CC3CC3)[C@@H]3CC[C@@H](NC4COC4)OC3)C2=O)C(=O)N1] | 3-(4-((cyclopropylmethyl)((1r,4r)-4- (oxetan-3-ylamino)cyclohexyl)amino)- 1-oxoisoindolin-2-yl)piperidine-2,6-dione |
| 535 | 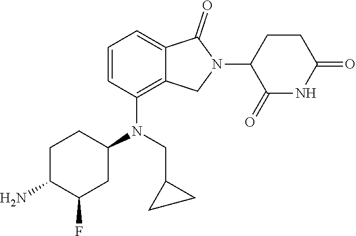 [SMILES: N[C@@H]1CC[C@@H](N(CC2CC2)c2cccc3c2CN(C2CCC(=O)NC2=O)C3=O)C[C@H]1F] | 3-(4-(((1R,3R,4R)-4-amino-3- fluorocyclohexyl)(cyclopropylmethyl) amino)-1-oxoisoindolin-2-yl)piperidine- 2,6-dione |
| 536 | 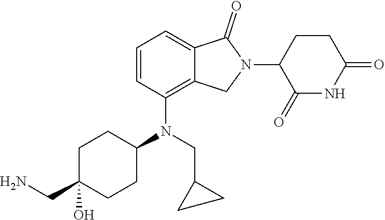 [SMILES: NC[C@]1(O)CC[C@H](N(CC2CC2)c2cccc3c2CN(C2CCC(=O)NC2=O)C3=O)CC1] | 3-(4-(((1r,4r)-4-(aminomethyl)-4- hydroxycyclohexyl)(cyclopropylmethyl) amino)-1-oxoisoindolin-2- yl)piperidine-2,6-dione |
| 539 | 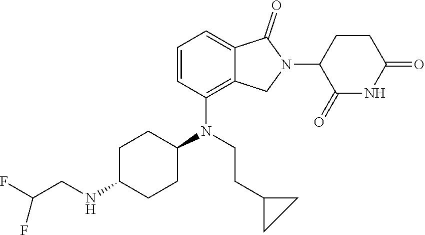 [SMILES: O=C1CCC(N2Cc3c(cccc3N(CCC3CC3)[C@H]3CC[C@H](NCC(F)F)CC3)C2=O)C(=O)N1] | 3-(4-((2-cyclopropylethyl)((1r,4r)-4- ((2,2- difluoroethyl)amino)cyclohexyl)amino)- 1-oxoisoindolin-2-yl)piperidine-2,6-dione |
| 539A | 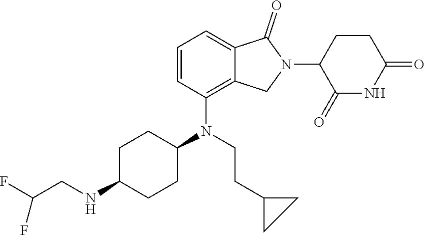 [SMILES: O=C1CCC(N2Cc3c(cccc3N(CCC3CC3)[C@H]3CC[C@@H](NCC(F)F)CC3)C2=O)C(=O)N1] | 3-(4-((2-cyclopropylethyl)((1s,4s)-4- ((2,2- difluoroethyl)amino)cyclohexyl)amino)- 1-oxoisoindolin-2-yl)piperidine-2,6-dione |
| 540 | 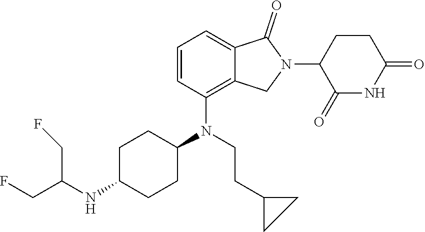 [SMILES: O=C1CCC(N2Cc3c(cccc3N(CCC3CC3)[C@H]3CC[C@H](NC(CF)CF)CC3)C2=O)C(=O)N1] | 3-(4-((2-cyclopropylethyl)((1r,4r)-4- ((1,3-difluoropropan-2- yl)amino)cyclohexyl)amino)-1- oxoisoindolin-2-yl)piperidine-2,6-dione |
| 541 | 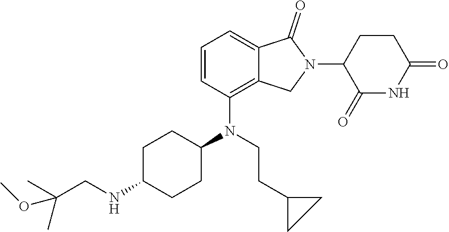 [SMILES: COC(C)(C)CN[C@H]1CC[C@H](N(CCC2CC2)c2cccc3c2CN(C2CCC(=O)NC2=O)C3=O)CC1] | 3-(4-((2-cyclopropylethyl)((1r,4r)-4- ((2-methoxy-2- methylpropyl)amino)cyclohexyl)amino)- 1-oxoisoindolin-2-yl)piperidine-2,6-dione |
| 541A | 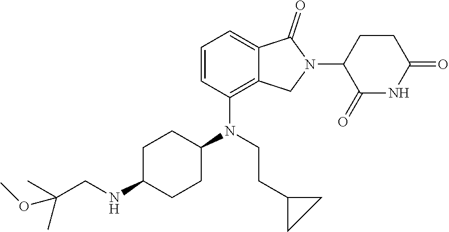 [SMILES: COC(C)(C)CN[C@H]1CC[C@@H](N(CCC2CC2)c2cccc3c2CN(C2CCC(=O)NC2=O)C3=O)CC1] | 3-(4-((2-cyclopropylethyl)((1s,4s)-4- ((2-methoxy-2- methylpropyl)amino)cyclohexyl)amino)- 1-oxoisoindolin-2-yl)piperidine-2,6-dione |
| 542 | 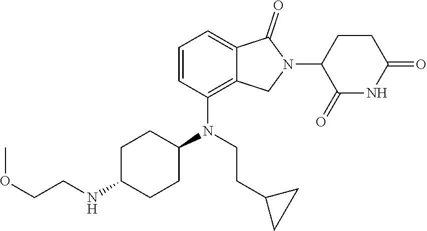 [SMILES: COCCN[C@H]1CC[C@H](N(CCC2CC2)c2cccc3c2CN(C2CCC(=O)NC2=O)C3=O)CC1] | 3-(4-((2-cyclopropylethyl)((1r,4r)-4- ((2- methoxyethyl)amino)cyclohexyl)amino)-1- oxoisoindolin-2-yl)piperidine-2,6-dione |
| 543 | 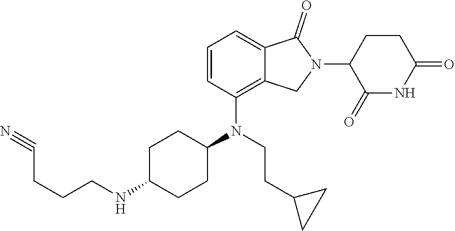 [SMILES: N#CCCCN[C@H]1CC[C@H](N(CCC2CC2)c2cccc3c2CN(C2CCC(=O)NC2=O)C3=O)CC1] | 4-(((1r,4r)-4-((2-cyclopropylethyl)(2- (2,6-dioxopiperidin-3-yl)-1- oxoisoindolin-4-yl)amino)cyclohexyl)amino) butanenitrile |
| 546 | 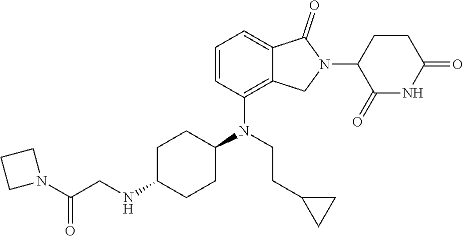 [SMILES: O=C1CCC(N2Cc3c(cccc3N(CCC3CC3)[C@H]3CC[C@H](NCC(=O)N4CCC4)CC3)C2=O)C(=O)N1] | 3-(4-(((1r,4r)-4-((2-(azetidin-1-yl)-2- oxoethyl)amino)cyclohexyl)(2- cyclopropylethyl)amino)-1- oxoisoindolin-2-yl)piperidine-2,6-dione |
| 547 | 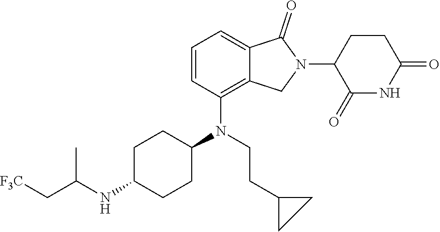 [SMILES: CC(CC(F)(F)F)N[C@H]1CC[C@H](N(CCC2CC2)c2cccc3c2CN(C2CCC(=O)NC2=O)C3=O)CC1] | 3-(4-((2-cyclopropylethyl)((1r,4r)-4- ((4,4,4-trifluorobutan-2- yl)amino)cyclohexyl)amino)-1- oxoisoindolin-2-yl)piperidine-2,6-dione |
| 548 | 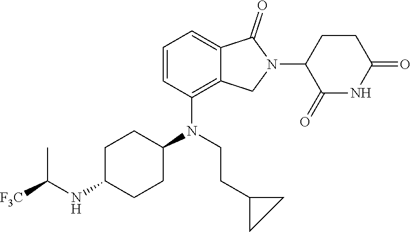 [SMILES: C[C@@H](N[C@H]1CC[C@H](N(CCC2CC2)c2cccc3c2CN(C2CCC(=O)NC2=O)C3=O)CC1)C(F)(F)F] | 3-(4-((2-cyclopropylethyl)((1R,4r)-4- (((R)-1,1-trifluoropropan-2- yl)amino)cyclohexyl)amino)-1- oxoisoindolin-2-yl)piperidine-2,6-dione |
| 548A | 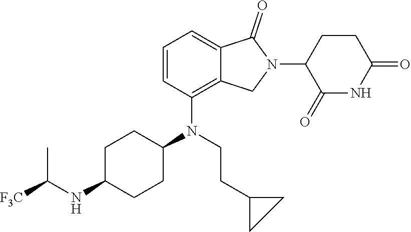 [SMILES: C[C@@H](N[C@H]1CC[C@@H](N(CCC2CC2)c2cccc3c2CN(C2CCC(=O)NC2=O)C3=O)CC1)C(F)(F)F] | 3-(4-((2-cyclopropylethyl)((1S,4s)-4- (((R)-1,1,1-trifluoropropan-2- yl)amino)cyclohexyl)amino)-1- oxoisoindolin-2-yl)piperidine-2,6-dione |
| 549 | 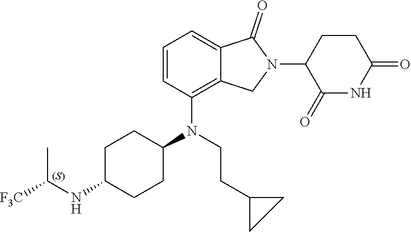 | 3-(4-((2-cyclopropylethyl)((1S,4r)-4- (((S)-1,1,1-trifluoropropan-2- yl)amino)cyclohexyl)amino)-1- oxoisoindolin-2-yl)piperidine-2,6-dione |
| 550 | 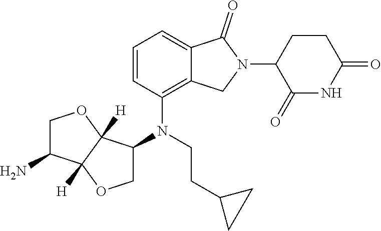 [SMILES: N[C@H]1CO[C@H]2[C@@H]1OC[C@@H]2N(CCC1CC1)c1cccc2c1CN(C1CCC(=O)NC1=O)C2=O] | 3-(4-(((3S,3aR,6S,6aR)-6- aminohexahydrofuro[3,2-b]furan-3- yl)(2-cyclopropylethyl)amino)-1- oxoisoindolin-2-yl)piperidine-2,6-dione |
| 552 | 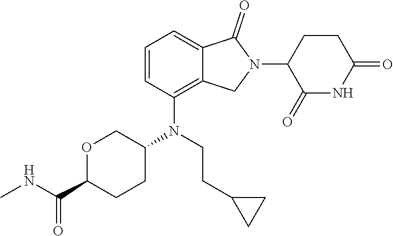 [SMILES: CNC(=O)[C@@H]1CC[C@@H](N(CCC2CC2)c2cccc3c2CN(C2CCC(=O)NC2=O)C3=O)CO1] | (2S,5R)-5-((2-cyclopropylethyl)(2- (2,6-dioxopiperidin-3-yl)-1- oxoisoindolin-4-yl)amino)-N- methyltetrahydro-2H-pyran-2- carboxamide |
| 554 | 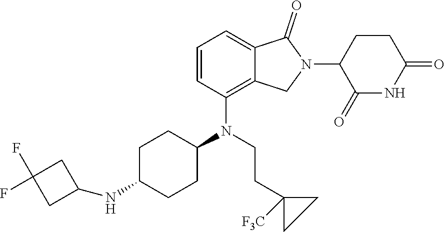 [SMILES: O=C1CCC(N2Cc3c(cccc3N(CCC3(C(F)(F)F)CC3)[C@H]3CC[C@H](NC4CC(F)(F)C4)CC3)C2=O)C(=O)N1] | 3-(4-(((1r,4r)-4-((3,3- difluorocyclobutyl)amino)cyclohexyl) (2-(1- (trifluoromethyl)cyclopropyl)ethyl) amino)-1-oxoisoindolin-2-yl)piperidine- 2,6-dione |
| 554A | 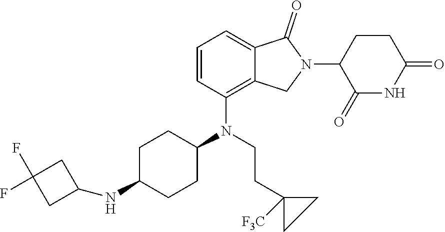 [SMILES: O=C1CCC(N2Cc3c(cccc3N(CCC3(C(F)(F)F)CC3)[C@H]3CC[C@@H](NC4CC(F)(F)C4)CC3)C2=O)C(=O)N1] | 3-(4-(((1s,4s)-4-((3,3- difluorocyclobutyl)amino)cyclohexyl)(2-(1- (trifluoromethyl)cyclopropyl)ethyl) amino)-1-oxoisoindolin-2-yl)piperidine- 2,6-dione |
| 557 | 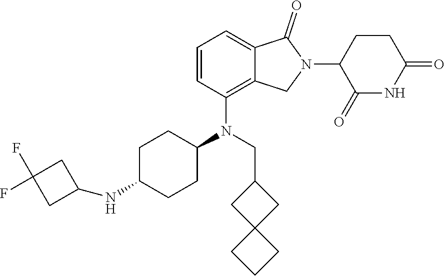 [SMILES: O=C1CCC(N2Cc3c(cccc3N(CC3CC4(CCC4)C3)[C@H]3CC[C@H](NC4CC(F)(F)C4)CC3)C2=O)C(=O)N1] | 3-(4-(((1r,4r)-4-((3,3- difluorobutyl)amino)cyclohexyl) (spiro[3.3]heptan-2-ylmethyl)amino)-1- oxoisoindolin-2-yl)piperidine-2,6-dione |
| 558-2 | 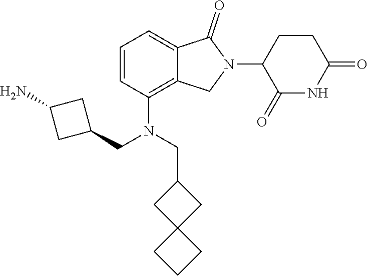 [SMILES: N[C@H]1C[C@H](CN(CC2CC3(CCC3)C2)c2cccc3c2CN(C2CCC(=O)NC2=O)C3=O)C1] | 3-(4-((((1r,3r)-3- aminocyclobutyl)methyl)(spiro [3.3]heptan-2-ylmethyl)amino)-1- oxoisoindolin-2-yl)piperidine-2,6-dione |
| 561 | 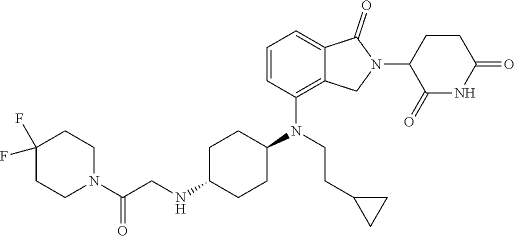 [SMILES: O=C1CCC(N2Cc3c(cccc3N(CCC3CC3)[C@H]3CC[C@H](NCC(=O)N4CCC(F)(F)CC4)CC3)C2=O)C(=O)N1] | 3-(4-((2-cyclopropylethyl)((1r,4r)-4- ((2-(4,4-difluoropiperidin-1-yl)-2- oxoethyl)amino)cyclohexyl)amino)-1- oxoisoindolin-2-yl)piperidine-2,6-dione |
| 562 | 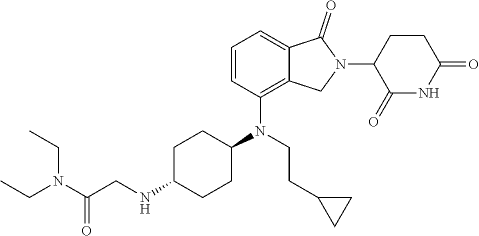 [SMILES: CCN(CC)C(=O)CN[C@H]1CC[C@H](N(CCC2CC2)c2cccc3c2CN(C2CCC(=O)NC2=O)C3=O)CC1] | 2-(((1r,4r)-4-((2-cyclopropylethyl)(2- (2,6-dioxopiperidin-3-yl)-1- oxoisoindolin-4- yl)amino)cyclohexyl)amino)-N,N- diethylacetamide |
| 573 | 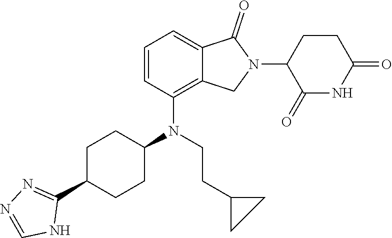 [SMILES: O=C1CCC(N2Cc3c(cccc3N(CCC3CC3)[C@H]3CC[C@@H](c4nnc[nH]4)CC3)C2=O)C(=O)N1] | 3-(4-(((1s,4s)-4-(2H-1,2,4-triazol-3- yl)cyclohexyl)(2- cyclopropylethyl)amino)-1- oxoisoindolin-2-yl)piperidine-2,6-dione |
| 574 | 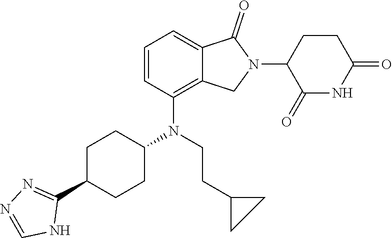 [SMILES: O=C1CCC(N2Cc3c(cccc3N(CCC3CC3)[C@H]3CC[C@H](c4nnc[nH]4)CC3)C2=O)C(=O)N1] | 3-(4-(((1r,4r)-4-(4H-1,2,4-triazol-3- yl)cyclohexyl)(2- cyclopropylethyl)amino)-1- oxoisoindolin-2-yl)piperidine-2,6-dione |
| 575 | 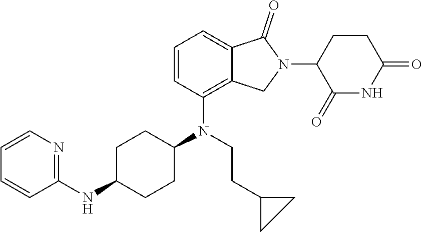 [SMILES: O=C1CCC(N2Cc3c(cccc3N(CCC3CC3)[C@H]3CC[C@@H](Nc4ccccn4)CC3)C2=O)C(=O)N1] | 3-(4-((2-cyclopropylethyl)((1s,4s)-4- (pyridin-2- ylamino)cyclohexyl)amino)-1- oxoisoindolin-2-yl)piperidine-2,6-dione |
| 576 | 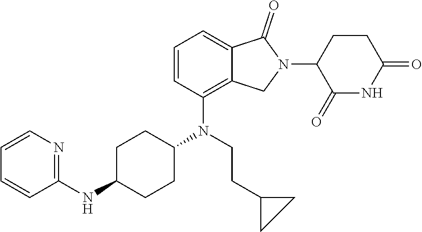 [SMILES: O=C1CCC(N2Cc3c(cccc3N(CCC3CC3)[C@H]3CC[C@H](Nc4ccccn4)CC3)C2=O)C(=O)N1] | 3-(4-((2-cyclopropylethyl)((1r,4r)-4- (pyridin-2- ylamino)cyclohexyl)amino)-1- oxoisoindolin-2-yl)piperidine-2,6-dione |
| 577 | 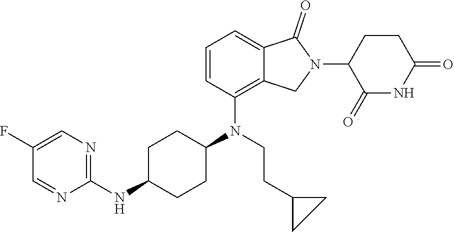 [SMILES: O=C1CCC(N2Cc3c(cccc3N(CCC3CC3)[C@H]3CC[C@@H](Nc4ncc(F)cn4)CC3)C2=O)C(=O)N1] | 3-(4-((2-cyclopropylethyl)((1s,4s)-4- ((5-fluoropyrimidin-2- yl)amino)cyclohexyl)- amino)-1- oxoisoindolin-2-yl)piperidine-2,6-dione |
| 578 | 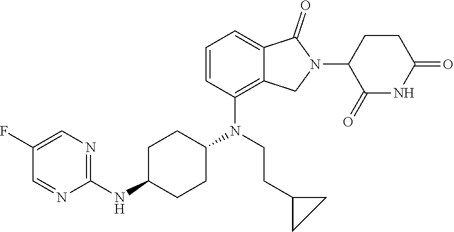 [SMILES: O=C1CCC(N2Cc3c(cccc3N(CCC3CC3)[C@H]3CC[C@H](Nc4ncc(F)cn4)CC3)C2=O)C(=O)N1] | 3-(4-((2-cyclopropylethyl)((1r,4r)-4- ((5-fluoropyrimidin-2- yl)amino)cyclohexyl)amino)-1- oxoisoindolin-2-yl)piperidine-2,6-dione |
| 580 | 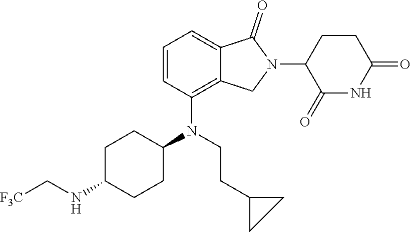 [SMILES: O=C1CCC(N2Cc3c(cccc3N(CCC3CC3)[C@H]3CC[C@H](NCC(F)(F)F)CC3)C2=O)C(=O)N1] | 3-(4-((2-cyclopropylethyl)((1r,4r)-4- ((2,2,2- trifluoroethyl)amino)cyclohexyl)amino)- 1-oxoisoindolin-2-yl)piperidine-2,6-dione |
| 581 | 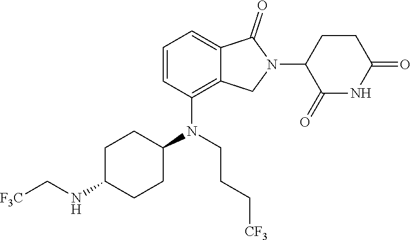 [SMILES: O=C1CCC(N2Cc3c(cccc3N(CCCC(F)(F)F)[C@H]3CC[C@H](NCC(F)(F)F)CC3)C2=O)C(=O)N1] | 3-(1-oxo-4-((4,4,4,- trifluorobutyl)((1r,4r)-4-((2,2,2- trifluoroethyl)amino)cyclohexyl)amino) isoindolin-2-yl)piperidine-2,6-dione |
| 582 | 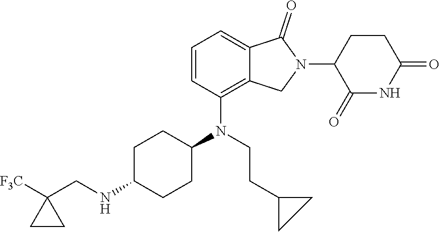 [SMILES: O=C1CCC(N2Cc3c(cccc3N(CCC3CC3)[C@H]3CC[C@H](NCC4(C(F)(F)F)CC4)CC3)C2=O)C(=O)N1] | 3-(4-((2-cyclopropylethyl)((1r,4r)-4- (((1- (trifluoromethyl)cyclopropyl)methyl) amino)cyclohexyl)amino)-1- oxoisoindolin-2-yl)piperidine-2,6-dione |
| 583 | 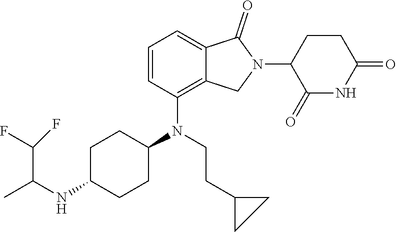 [SMILES: CC(N[C@H]1CC[C@H](N(CCC2CC2)c2cccc3c2CN(C2CCC(=O)NC2=O)C3=O)CC1)C(F)F] | 3-(4-((2-cyclopropylethyl)((1r,4r)-4- ((1,1-difluoropropan-2- yl)amino)cyclohexyl)amino)-1- oxoisoindolin-2-yl)piperidine-2,6-dione |
| 603 | 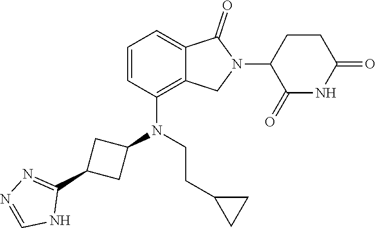 [SMILES: O=C1CCC(N2Cc3c(cccc3N(CCC3CC3)[C@H]3C[C@@H](c4nnc[nH]4)C3)C2=O)C(=O)N1] | 3-(4-(((1s,3s)-3-(4H-1,2,4-triazol-3- yl)cyclobutyl)(2- cyclopropylethyl)amino)-1- oxoisoindolin-2-yl)piperidine-2,6-dione |
| 604 | 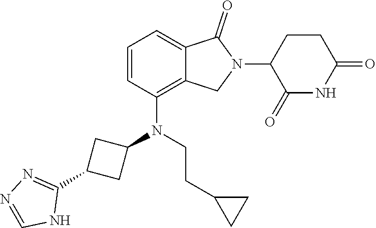 [SMILES: O=C1CCC(N2Cc3c(cccc3N(CCC3CC3)[C@H]3C[C@H](c4nnc[nH]4)C3)C2=O)C(=O)N1] | 3-(4-(((1r,3r)-3-(4H-1,2,4-triazol-3- yl)cyclobutyl)(2- cyclopropylethyl)amino)-1- oxoisoindolin-2-yl)piperidine-2,6-dione |
| 611 | 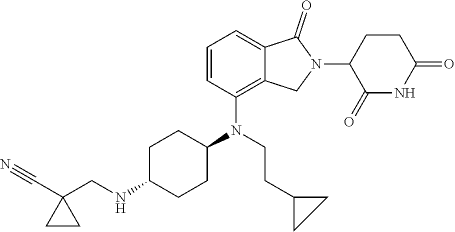 [SMILES: N#CC1(CN[C@H]2CC[C@H](N(CCC3CC3)c3cccc4c3CN(C3CCC(=O)NC3=O)C4=O)CC2)CC1] | 1-((((1r,4r)-4-((2-cyclopropylethyl)(2- (2,6-dioxopiperidin-3-yl)-1- oxoisoindolin-4- yl)amino)cyclohexyl)amino)methyl) cyclopropanecarbonitrile |
| 612 | 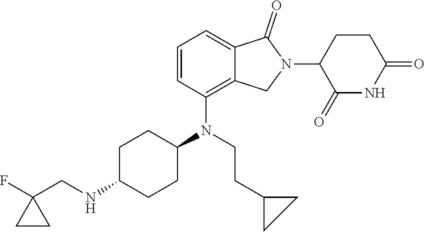 [SMILES: O=C1CCC(N2Cc3c(cccc3N(CCC3CC3)[C@H]3CC[C@H](NCC4(F)CC4)CC3)C2=O)C(=O)N1] | 3-(4-((2-cyclopropylethyl)((1r,4r)-4- (((1- fluorocyclopropyl)methyl)amino) cyclohexyl)amino)-1-oxoisoindolin-2- yl)piperidine-2,6-dione |
| 612A | 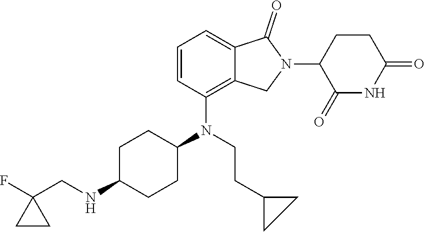 [SMILES: O=C1CCC(N2Cc3c(cccc3N(CCC3CC3)[C@H]3CC[C@@H](NCC4(F)CC4)CC3)C2=O)C(=O)N1] | 3-(4-((2-cyclopropylethyl)((1s,4s)-4- (((1- fluorocyclopropyl)methyl)amino) cyclohexyl)amino)-1-oxoisoindolin- 2-yl)piperidine-2,6-dione |
| 613 | 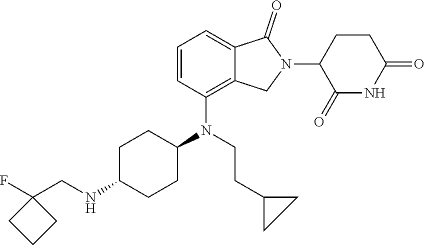 [SMILES: O=C1CCC(N2Cc3c(cccc3N(CCC3CC3)[C@H]3CC[C@H](NCC4(F)CCC4)CC3)C2=O)C(=O)N1] | 3-(4-((2-cyclopropylethyl)((1r,4r)-4- (((1- fluorocyclobutyl)methyl)amino) cyclohexyl)amino)-1-oxoisoindolin- 2-yl)piperidine-2,6-dione |
| 613A | 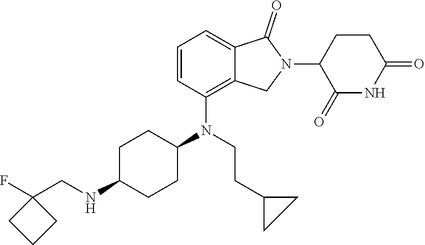 [SMILES: O=C1CCC(N2Cc3c(cccc3N(CCC3CC3)[C@H]3CC[C@@H](NCC4(F)CCC4)CC3)C2=O)C(=O)N1] | 3-(4-((2-cyclopropylethyl)((1s,4s)-4- (((1- fluorocyclobutyl)methyl)amino) cyclohexyl)amino)-1-oxoisoindolin-2- yl)piperidine-2,6-dione |
| 614 | 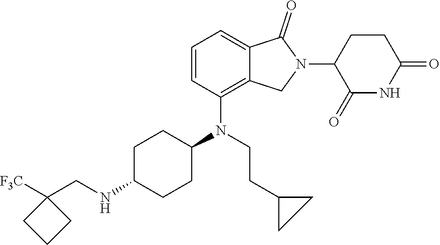 [SMILES: O=C1CCC(N2Cc3c(cccc3N(CCC3CC3)[C@H]3CC[C@H](NCC4(C(F)(F)F)CCC4)CC3)C2=O)C(=O)N1] | 3-(4-((2-cyclopropylethyl)((1r,4r)-4- (((1-(trifluoromethyl) cyclomethyl)cyclobutyl)methyl) amino)cyclohexyl)amino)-1- oxoisoindolin-2-yl)piperidine-2,6- dione |
| 614A | 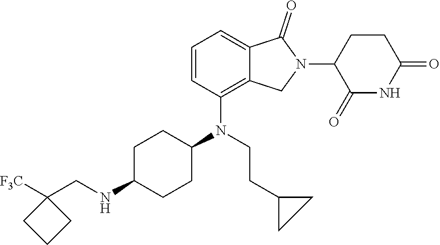 [SMILES: O=C1CCC(N2Cc3c(cccc3N(CCC3CC3)[C@H]3CC[C@@H](NCC4(C(F)(F)F)CCC4)CC3)C2=O)C(=O)N1] | 3-(4-((2-cyclopropylethyl)((1s,4s)-4- (((1- (trifluoromethyl)cyclobutyl)methyl) amino)cyclohexyl)amino)-1- oxoisoindolin-2-yl)piperidine-2,6-dione |
| 615 | 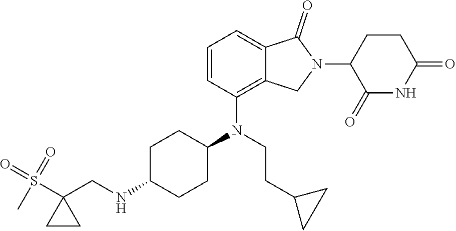 [SMILES: CS(=O)(=O)C1(CN[C@H]2CC[C@H](N(CCC3CC3)c3cccc4c3CN(C3CCC(=O)NC3=O)C4=O)CC2)CC1] | 3-(4-((2-cyclopropylethyl)((1r,4r)-4- (((1- (methylsulfonyl)cyclopropyl)methyl) amino)cyclohexyl)amino)-1- oxoisoindolin-2-yl)piperidine-2,6-dione |
| 615A | 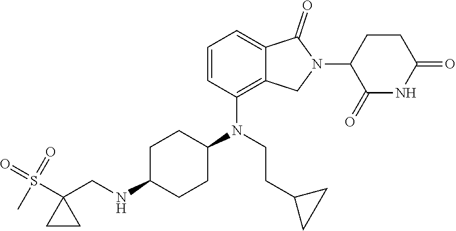 [SMILES: CS(=O)(=O)C1(CN[C@H]2CC[C@@H](N(CCC3CC3)c3cccc4c3CN(C3CCC(=O)NC3=O)C4=O)CC2)CC1] | 3-(4-((2-cyclopropylethyl)((1s,4s)-4- (((1- (methylsulfonyl)cyclopropyl)methyl) amino)cyclohexyl)amino)-1- oxoisoindolin-2-yl)piperidine-2,6-dione |
| 616 | 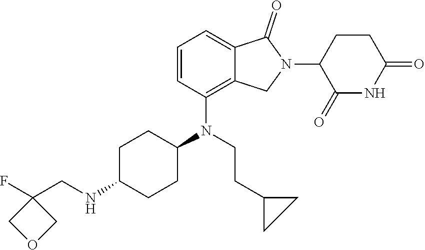 [SMILES: O=C1CCC(N2Cc3c(cccc3N(CCC3CC3)[C@H]3CC[C@H](NCC4(F)COC4)CC3)C2=O)C(=O)N1] | 3-(4-((2-cyclopropylethyl)((1r,4r)-4- (((3-fluorooxetan-3- yl)methyl)amino)cyclohexyl)amino)-1- oxoisoindolin-2-yl)piperidine-2,6-dione |
| 616A | 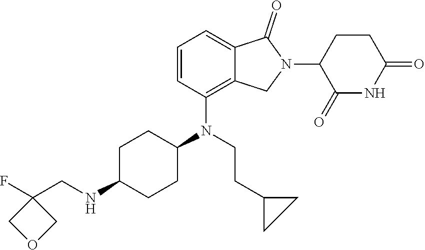 [SMILES: O=C1CCC(N2Cc3c(cccc3N(CCC3CC3)[C@H]3CC[C@@H](NCC4(F)COC4)CC3)C2=O)C(=O)N1] | 3-(4-((2-cyclopropylethyl)((1s,4s)-4- (((3-fluorooxetan-3- yl)methyl)amino)cyclohexyl)amino)-1- oxoisoindolin-2-yl)piperidine-2,6-dione |
| 619 | 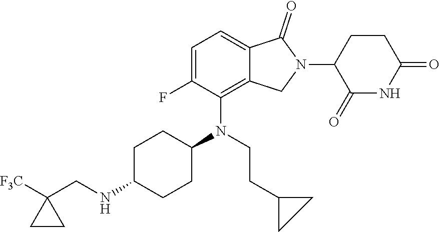 [SMILES: O=C1CCC(N2Cc3c(ccc(F)c3N(CCC3CC3)[C@H]3CC[C@H](NCC4(C(F)(F)F)CC4)CC3)C2=O)C(=O)N1] | 3-(4-((2-cyclopropylethyl)((1r,4r)-4- (((1- trifluoromethyl)cyclopropyl)methyl) amino)cyclohexyl)amino)-5-fluoro-1- oxoisoindolin-2-yl)piperidine-2,6-dione |
| 627 | 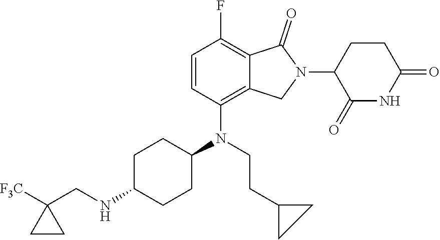 [SMILES: O=C1CCC(N2Cc3c(N(CCC4CC4)[C@H]4CC[C@H](NCC5(C(F)(F)F)CC5)CC4)ccc(F)c3C2=O)C(=O)N1] | 3-(4-((2-cyclopropylethyl)((1r,4r)-4- (((1- (trifluoromethyl)cyclopropyl)methyl) amino)cyclohexyl)amino)-7-fluoro-1- oxoisoindolin-2-yl)piperidine-2,6- dione |
| 627A | 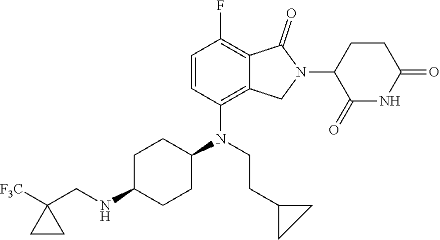 [SMILES: O=C1CCC(N2Cc3c(N(CCC4CC4)[C@H]4CC[C@@H](NCC5(C(F)(F)F)CC5)CC4)ccc(F)c3C2=O)C(=O)N1] | 3-(4-((2-cyclopropylethyl)((1s,4s)-4- (((1- (trifluoromethyl)cyclopropyl)methyl) amino)cyclohexyl)amino)-7-fluoro-1- oxoisoindolin-2-yl)piperidine-2,6-dione |
| 628 | 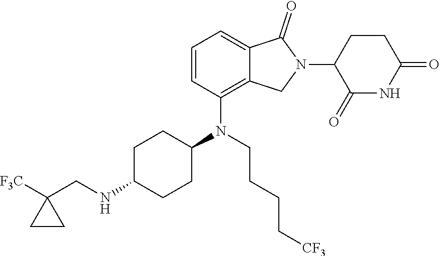 [SMILES: O=C1CCC(N2Cc3c(cccc3N(CCCCC(F)(F)F)[C@H]3CC[C@H](NCC4(C(F)(F)F)CC4)CC3)C2=O)C(=O)N1] | 3-(1-oxo-4-(((1r,4r)-4-(((1- (trifluoromethyl)cyclopropyl)methyl) amino)cyclohexyl)(5,5,5- trifluoropentyl)amino)isoindolin-2- yl)piperidine-2,6-dione |
| 628A | 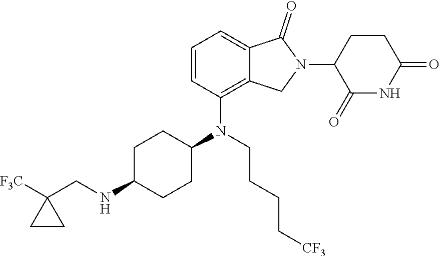 [SMILES: O=C1CCC(N2Cc3c(cccc3N(CCCCC(F)(F)F)[C@H]3CC[C@@H](NCC4(C(F)(F)F)CC4)CC3)C2=O)C(=O)N1] | 3-(1-oxo-4-(((1s,4s)-4-(((1- (trifluoromethyl)cyclopropyl)methyl) amino)cyclohexyl)(5,5,5- trifluoropentyl)amino)isoindolin-2- yl)piperidine-2,6-dione |
| 629 | 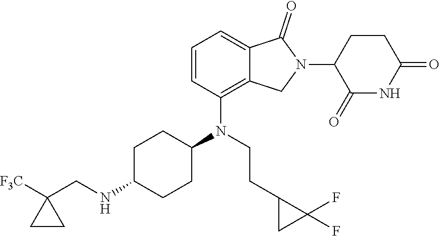 [SMILES: O=C1CCC(N2Cc3c(cccc3N(CCC3CC3(F)F)[C@H]3CC[C@H](NCC4(C(F)(F)F)CC4)CC3)C2=O)C(=O)N1] | 3-(4-((2-(2,2- difluorocyclopropyl)ethyl)((1r,4r)-4- (((1- (trifluoromethyl)cyclopropyl)methyl) amino)cyclohexyl)amino)-1- oxoisoindolin-2-yl)piperidine-2,6-dione |
| 629A | 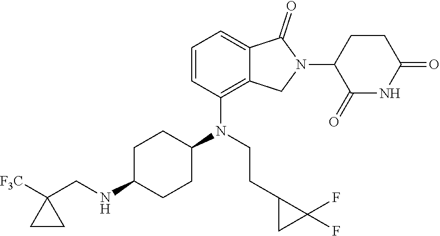 [SMILES: O=C1CCC(N2Cc3c(cccc3N(CCC3CC3(F)F)[C@H]3CC[C@@H](NCC4(C(F)(F)F)CC4)CC3)C2=O)C(=O)N1] | 3-(4-((2-(2,2- difluorocyclopropyl)ethyl)((1s,4s)-4- (((1- (trifluoromethyl)cyclopropyl)methyl) amino)cyclohexyl)amino)-1- oxoisoindolin-2-yl)piperidine-2,6-dione |
| 630 | 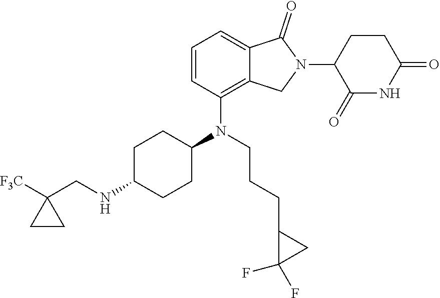 [SMILES: O=C1CCC(N2Cc3c(cccc3N(CCCC3CC3(F)F)[C@H]3CC[C@H](NCC4(C(F)(F)F)CC4)CC3)C2=O)C(=O)N1] | 3-(4-((3-(2,2- difluorocyclopropyl)propyl)((1r,4r)-4- (((1- (trifluoromethyl)cyclopropyl)methyl) amino)cyclohexyl)amino)-1- oxoisoindolin-2-yl)piperidine-2,6-dione |
| 630A | 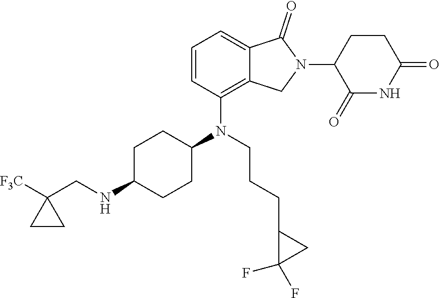 [SMILES: O=C1CCC(N2Cc3c(cccc3N(CCCC3CC3(F)F)[C@H]3CC[C@@H](NCC4(C(F)(F)F)CC4)CC3)C2=O)C(=O)N1] | 3-(4-((3-(2,2- difluorocyclopropyl)propyl)((1s,4s)-4- (((1- (trifluoromethyl)cyclopropyl)methyl) amino)cyclohexyl)amino)-1- oxoisoindolin-2-yl)piperidine-2,6-dione |
| 634 | 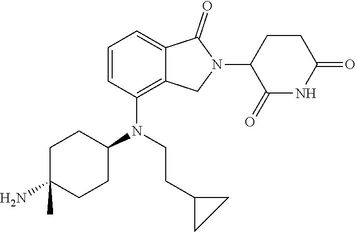 [SMILES: C[C@]1(N)CC[C@H](N(CCC2CC2)c2cccc3c2CN(C2CCC(=O)NC2=O)C3=O)CC1] | 3-(4-(((1r,4r)-4-amino-4- methylcyclohexyl)(2- cyclopropylethyl)amino)-1- oxoisoindolin-2-yl)piperidine-2,6-dione |
| 635 | 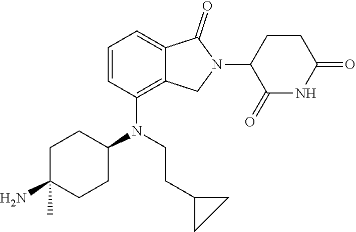 [SMILES: C[C@]1(N)CC[C@@H](N(CCC2CC2)c2cccc3c2CN(C2CCC(=O)NC2=O)C3=O)CC1] | 3-(4-(((1s,4s)-4-amino-4- methylcyclohexyl)(2- cyclopropylethyl)amino)-1- oxoisoindolin-2-yl)piperidine-2,6-dione |
| 636 | 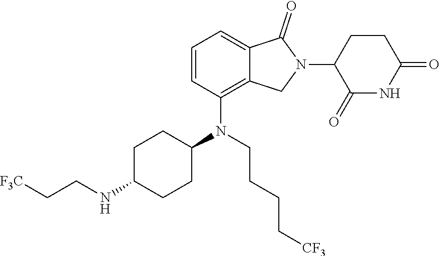 [SMILES: O=C1CCC(N2Cc3c(cccc3N(CCCCC(F)(F)F)[C@H]3CC[C@H](NCCC(F)(F)F)CC3)C2=O)C(=O)N1] | 3-(1-oxo-4-((5,5,5- trifluoropentyl)((1r,4r)-4-((3,3,3- trifluoropropyl)amino)cyclohexyl) amino)isoindolin-2-yl)piperidine-2,6-dione |
| 636A | 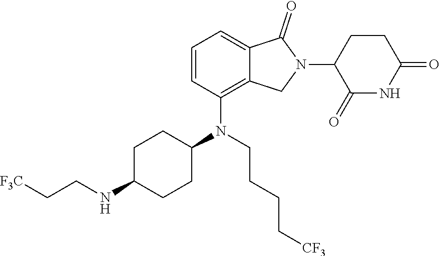 [SMILES: O=C1CCC(N2Cc3c(cccc3N(CCCCC(F)(F)F)[C@H]3CC[C@@H](NCCC(F)(F)F)CC3)C2=O)C(=O)N1] | 3-(1-oxo-4-((5,5,5- trifluoropentyl)((1s,4s)-4-((3,3,3- trifluoropropyl)amino)cyclohexyl) amino)isoindolin-2-yl)piperidine-2,6-dione |
| 637 | 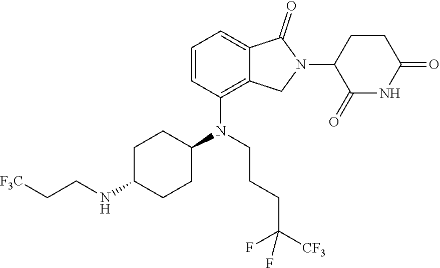 [SMILES: O=C1CCC(N2Cc3c(cccc3N(CCCC(F)(F)C(F)(F)F)[C@H]3CC[C@H](NCCC(F)(F)F)CC3)C2=O)C(=O)N1] | 3-(1-oxo-4-((4,4,5,5,5- pentafluoropentyl)((1r,4r)-4-((3,3,3- trifluoropropyl)amino)cyclohexyl) amino)isoindolin-2-yl)piperidine-2,6-dione |
| 637A | 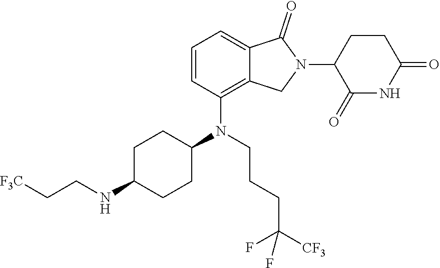 [SMILES: O=C1CCC(N2Cc3c(cccc3N(CCCC(F)(F)C(F)(F)F)[C@H]3CC[C@@H](NCCC(F)(F)F)CC3)C2=O)C(=O)N1] | 3-(1-oxo-4-((4,4,5,5,5- pentafluoropentyl)((1s,4s)-4-((3,3,3- trifluoropropyl)amino)cyclohexyl)amino)- isoindolin-2-yl)piperidine-2,6-dione |
| 639 | 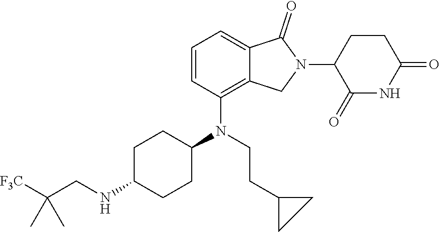 [SMILES: CC(C)(CN[C@H]1CC[C@H](N(CCC2CC2)c2cccc3c2CN(C2CCC(=O)NC2=O)C3=O)CC1)C(F)(F)F] | 3-(4-((2-cyclopropylethyl)((1r,4r)-4- (3,3,3-trifluoro-2,2- dimethylpropyl)amino)cyclohexyl) amino)-1-oxoisoindolin-2-yl)piperidine- 2,6-dione |
| 639A | 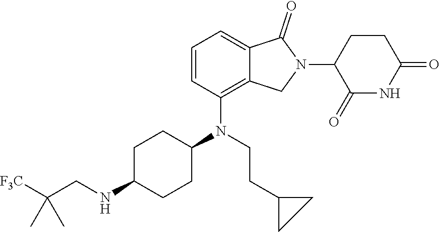 [SMILES: CC(C)(CN[C@H]1CC[C@@H](N(CCC2CC2)c2cccc3c2CN(C2CCC(=O)NC2=O)C3=O)CC1)C(F)(F)F] | 3-(4-((2-cyclopropylethyl)((1s,4s)-4- ((3,3,3-trifluoro-2,2- dimethylpropyl)amino)cyclohexyl) amino)-1-oxoisoindolin-2-yl)piperidine- 2,6-dione |
| 642 | 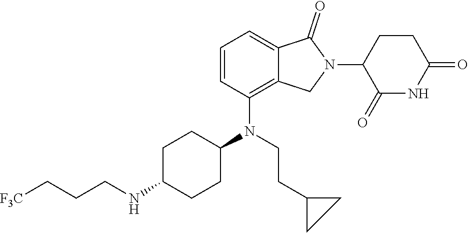 [SMILES: O=C1CCC(N2Cc3c(cccc3N(CCC3CC3)[C@H]3CC[C@H](NCCCC(F)(F)F)CC3)C2=O)C(=O)N1] | 3-(4-((2-cyclopropylethyl)((1r,4r)-4- ((4,4,4- trifluorobutyl)amino)cyclohexyl) amino)-1-oxoisoindolin-2-yl)piperidine- 2,6-dione |
| 642A | 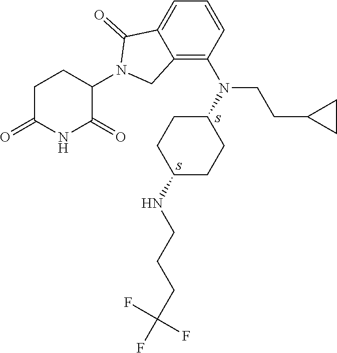 | 3-(4-((2-cyclopropylethyl)((1s,4s)-4- ((4,4,4- trifluorobutyl)amino)cyclohexyl)amino)- 1-oxoisoindolin-2-yl)piperidine-2,6-dione |
| 643 | 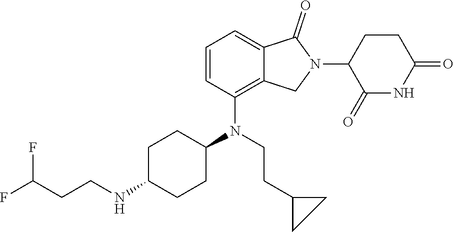 [SMILES: O=C1CCC(N2Cc3c(cccc3N(CCC3CC3)[C@H]3CC[C@H](NCCC(F)F)CC3)C2=O)C(=O)N1] | 3-(4-((2-cyclopropylethyl)((1r,4r)-4- ((3,3- difluoropropyl)amino)cyclohexyl)amino)- 1-oxoisoindolin-2-yl)piperidine-2,6-dione |
| 643A | 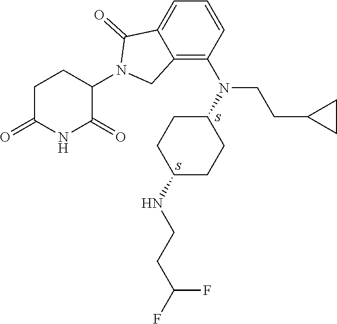 | 3-(4-((2-cyclopropylethyl)((1s,4s)-4- ((3,3- difluoropropyl)amino)cyclohexyl)amino)-1- oxoisoindolin-2-yl)piperidine-2,6-dione |
| 644 | 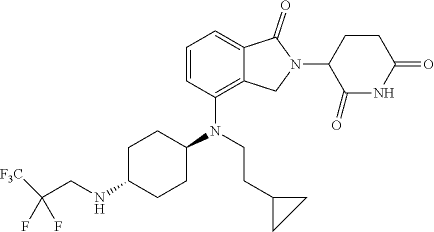 [SMILES: O=C1CCC(N2Cc3c(cccc3N(CCC3CC3)[C@H]3CC[C@H](NCC(F)(F)C(F)(F)F)CC3)C2=O)C(=O)N1] | 3-(4-((2-cyclopropylethyl)((1r,4r)-4- (2,2,3,3,3- pentafluoropropyl)amino)cyclohexyl) amino)-1-oxoisoindolin-2-yl)piperidine- 2,6-dione |
| 644A | 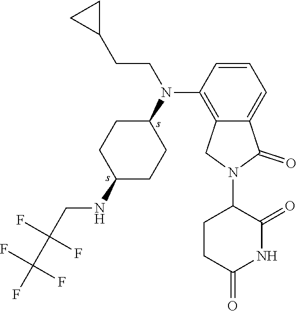 [SMILES: O=C1CCC(N2Cc3c(cccc3N(CCC3CC3)[C@H]3CC[C@@H](NCC(F)(F)C(F)(F)F)CC3)C2=O)C(=O)N1] | 3-(4-((2-cyclopropylethyl)((1s,4s)-4- (2,2,3,3,3- pentafluoropropyl)amino)cyclohexyl) amino)-1-oxoisoindolin-2-yl)piperidine- 2,6-dione |
| 645 | 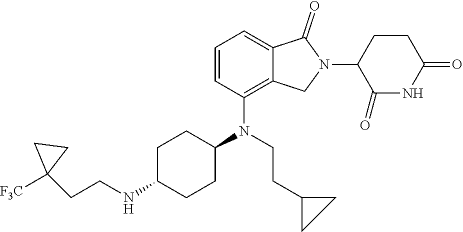 [SMILES: O=C1CCC(N2Cc3c(cccc3N(CCC3CC3)[C@H]3CC[C@H](NCCC4(C(F)(F)F)CC4)CC3)C2=O)C(=O)N1] | 3-(4-((2-cyclopropylethyl)((1r,4r)-4- ((2-(1- (trifluoromethyl)cyclopropyl)ethyl) amino)cyclohexyl)amino)-1- oxoisoindolin-2-yl)piperidine-2,6- dione |
| 645A | 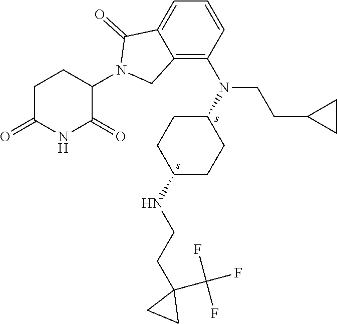 [SMILES: O=C1CCC(N2Cc3c(cccc3N(CCC3CC3)[C@H]3CC[C@@H](NCCC4(C(F)(F)F)CC4)CC3)C2=O)C(=O)N1] | 3-(4-((2-cyclopropylethyl)((1s,4s)-4- ((2-(1- (trifluoromethyl)cyclopropyl)ethyl) amino)cyclohexyl)amino)-1- oxoisoindolin-2-yl)piperidine-2,6-dione |
| 646 | 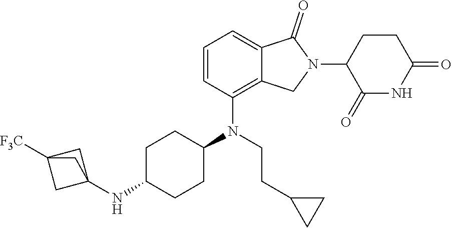 [SMILES: O=C1CCC(N2Cc3c(cccc3N(CCC3CC3)[C@H]3CC[C@H](NC45CC(C(F)(F)F)(C4)C5)CC3)C2=O)C(=O)N1] | 3-(4-((2-cyclopropylethyl)((1r,4r)-4- ((3- (trifluoromethyl)bicyclo[1.1.1]pentan- 1-yl)amino)cyclohexyl)amino)-1- oxoisoindolin-2-yl)piperidine-2,6- dione |
| 646A | 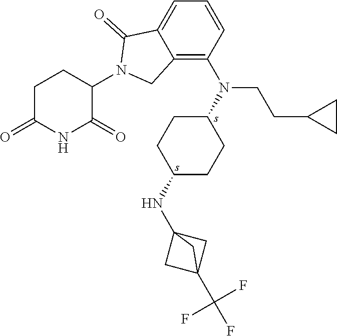 [SMILES: O=C1CCC(N2Cc3c(cccc3N(CCC3CC3)[C@H]3CC[C@@H](NC45CC(C(F)(F)F)(C4)C5)CC3)C2=O)C(=O)N1] | 3-(4-((2-cyclopropylethyl)((1s,4s)-4- ((3-(trifluoromethyl)bicyclo[1.1.1]pentan- 1-yl)amino)cyclohexyl)amino)-1- oxoisoindolin-2-yl)piperidine-2,6-dione |
| 647 | 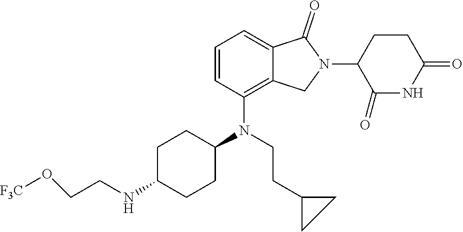 [SMILES: O=C1CCC(N2Cc3c(cccc3N(CCC3CC3)[C@H]3CC[C@H](NCCOC(F)(F)F)CC3)C2=O)C(=O)N1] | 3-(4-((2-cyclopropylethyl)((1r,4r)-4- ((2- (trifluoromethoxy)ethyl)amino)cyclohexyl) amino)-1-oxoisoindolin-2- yl)piperidine-2,6-dione |
| 647A | 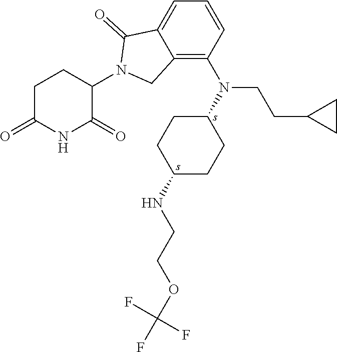 [SMILES: O=C1CCC(N2Cc3c(cccc3N(CCC3CC3)[C@H]3CC[C@@H](NCCOC(F)(F)F)CC3)C2=O)C(=O)N1] | 3-(4-((2-cyclopropylethyl)((1s,4s)-4- ((2- (trifluoromethoxy)ethyl)amino)cyclohexyl) amino)-1-oxoisoindolin-2-yl)piperidine- 2,6-dione |
| 648 | 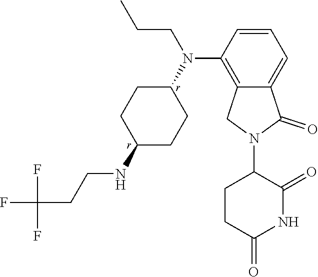 [SMILES: CCCN(c1cccc2c1CN(C1CCC(=O)NC1=O)C2=O)[C@H]1CC[C@H](NCCC(F)(F)F)CC1] | 3-(1-oxo-4-(propyl((1r,4r)-4-((3,3,3- trifluoropropyl)amino)cyclohexyl) amino)isoindolin-2-yl)piperidine-2,6-dione |
| 648A | 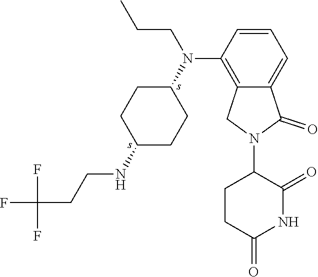 [SMILES: CCCN(c1cccc2c1CN(C1CCC(=O)NC1=O)C2=O)[C@H]1CC[C@@H](NCCC(F)(F)F)CC1] | 3-(1-oxo-4-(propyl((1s,4s)-4-((3,3,3- trifluoropropyl)amino)cyclohexyl) amino)isoindolin-2-yl)piperidine-2,6-dione |
| 649 | 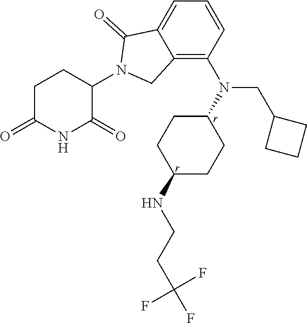 [SMILES: O=C1CCC(N2Cc3c(cccc3N(CC3CCC3)[C@H]3CC[C@H](NCCC(F)(F)F)CC3)C2=O)C(=O)N1] | 3-(4-((cyclobutylmethyl)((1r,4r)-4- ((3,3,3- trifluoropropyl)amino)cyclohexyl)amino)- 1-oxoisoindolin-2-yl)piperidine-2,6- dione |
| 649A | 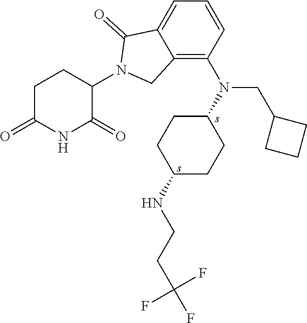 [SMILES: O=C1CCC(N2Cc3c(cccc3N(CC3CCC3)[C@H]3CC[C@@H](NCCC(F)(F)F)CC3)C2=O)C(=O)N1] | 3-(4-((cyclobutylmethyl)((1s,4s)-4- ((3,3,3- trifluoropropyl)amino)cyclohexyl)amino-1- oxoisoindolin-2-yl)piperidine-2,6-dione |
| 650 | 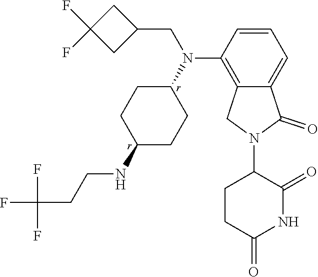 [SMILES: O=C1CCC(N2Cc3c(cccc3N(CC3CC(F)(F)C3)[C@H]3CC[C@H](NCCC(F)(F)F)CC3)C2=O)C(=O)N1] | 3-(4-(((3,3- difluorocyclobutyl)methyl)((1r,4r)-4- ((3,3,3- trifluoropropyl)amino)cyclohexyl)amino)- 1-oxoisoindolin-2-yl)piperidine-2,6- dione |
| 650A | 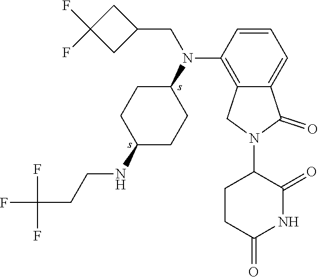 [SMILES: O=C1CCC(N2Cc3c(cccc3N(CC3CC(F)(F)C3)[C@H]3CC[C@@H](NCCC(F)(F)F)CC3)C2=O)C(=O)N1] | 3-(4-(((3,3- difluorocyclobutyl)methyl)((1s,4s)-4- ((3,3,3-trifluoropropyl)amino)cyclohexyl) amino)-1-oxoisoindolin-2-yl)piperidine- 2,6-dione |
| 651 | 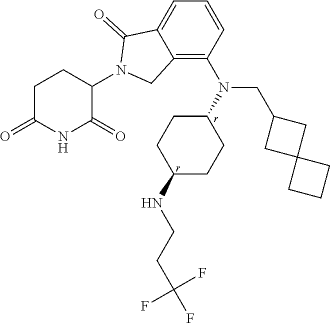 [SMILES: O=C1CCC(N2Cc3c(cccc3N(CC3CC4(CCC4)C3)[C@H]3CC[C@H](NCCC(F)(F)F)CC3)C2=O)C(=O)N1] | 3-(1-oxo-4-((spiro[3.3]heptan-2- ylmethyl)((1r,4r)-4-((3,3,3- trifluoropropyl)amino)cyclohexyl) amino)isoindolin-2-yl)piperidine-2,6-dione |
| 651A | 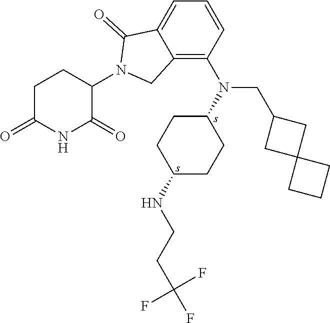 [SMILES: O=C1CCC(N2Cc3c(cccc3N(CC3CC4(CCC4)C3)[C@H]3CC[C@@H](NCCC(F)(F)F)CC3)C2=O)C(=O)N1] | 3-(1-oxo-4-(spiro[3.3]heptan-2- ylmethyl)((1s,4s)-4-(3,3,3- trifluoropropyl)amino)cyclohexyl)amino) isoindolin-2-yl)piperidine-2,6-dione |
| 652 | 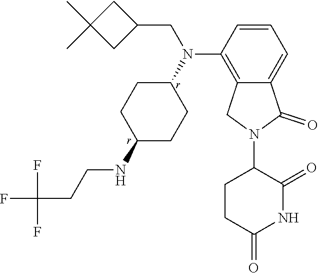 [SMILES: CC1(C)CC(CN(c2cccc3c2CN(C2CCC(=O)NC2=O)C3=O)[C@H]2CC[C@H](NCCC(F)(F)F)CC2)C1] | 3-(4-(((3,3- dimethylcyclobutyl)methyl)((1r,4r)-4- ((3,3,3- trifluoropropyl)amino)cyclohexyl) amino)-1-oxoisoindolin-2-yl)piperidine- 2,6-dione |
| 652A | 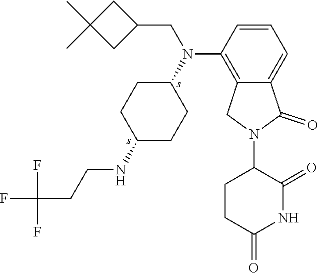 [SMILES: CC1(C)CC(CN(c2cccc3c2CN(C2CCC(=O)NC2=O)C3=O)[C@H]2CC[C@@H](NCCC(F)(F)F)CC2)C1] | 3-(4-(((3,3- dimethylcyclobutyl)methyl((1s,4s)-4- ((3,3,3- trifluoropropyl)amino)cyclohexyl) amino)-1-oxoisoindolin-2-yl)piperidine- 2,6-dione |
| 653 | 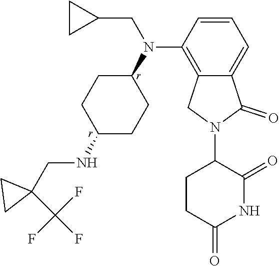 [SMILES: O=C1CCC(N2Cc3c(cccc3N(CC3CC3)[C@H]3CC[C@H](NCC4(C(F)(F)F)CC4)CC3)C2=O)C(=O)N1] | 3-(4-((cyclopropylmethyl)((1r,4r)-4- (((1-(trifluoromethyl)cyclopropyl)methyl) amino)cyclohexyl)amino)-1- oxoisoindolin-2-yl)piperidine-2,6-dione |
| 653A | 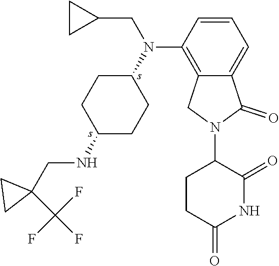 [SMILES: O=C1CCC(N2Cc3c(cccc3N(CC3CC3)[C@H]3CC[C@@H](NCC4(C(F)(F)F)CC4)CC3)C2=O)C(=O)N1] | 3-(4-((cyclopropylmethyl)((1s,4s)-4- (((1- (trifluoromethyl)cyclopropyl)methyl) amino)cyclohexyl)amino)-1- oxoisoindolin-2-yl)piperidine-2,6-dione |
| 654 | 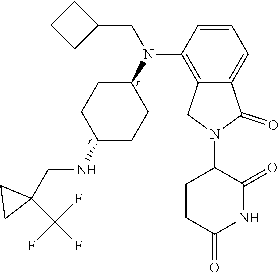 [SMILES: O=C1CCC(N2Cc3c(cccc3N(CC3CCC3)[C@H]3CC[C@H](NCC4(C(F)(F)F)CC4)CC3)C2=O)C(=O)N1] | 3-(4-((cyclobutylmethyl)((1r,4r)-4- (((1- (trifluoromethyl)cyclopropyl)methyl) amino)cyclohexyl)amino)-1- oxoisoindolin-2-yl)piperidine-2,6-dione |
| 654A | 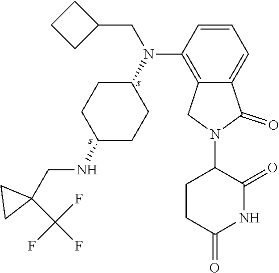 [SMILES: O=C1CCC(N2Cc3c(cccc3N(CC3CCC3)[C@H]3CC[C@@H](NCC4(C(F)(F)F)CC4)CC3)C2=O)C(=O)N1] | 3-(4-((cyclobutylmethyl)((1s,4s)-4- (((1- (trifluoromethyl)cyclopropyl)methyl) amino)cyclohexyl)amino)-1- oxoisoindolin-2-yl)piperidine-2,6-dione |
| 655 | 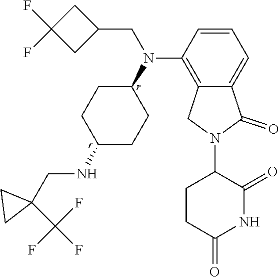 [SMILES: O=C1CCC(N2Cc3c(cccc3N(CC3CC(F)(F)C3)[C@H]3CC[C@H](NCC4(C(F)(F)F)CC4)CC3)C2=O)C(=O)N1] | 3-(4-(((3,3- difluorocyclobutyl)methyl)((1r,4r)-4- (((1- (trifluoromethyl)cyclopropyl)methyl) amino)cyclohexyl)amino)-1- oxoisoindolin-2-yl)piperidine-2,6-dione |
| 655A | 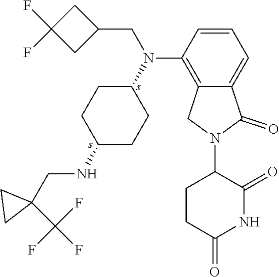 [SMILES: O=C1CCC(N2Cc3c(cccc3N(CC3CC(F)(F)C3)[C@H]3CC[C@@H](NCC4(C(F)(F)F)CC4)CC3)C2=O)C(=O)N1] | 3-(4-(((3,3- difluorocyclobutyl)methyl)((1s,4s)-4- (((1- (trifluoromethyl)cyclopropyl)methyl) amino)cyclohexyl)amino)-1- oxoisoindolin-2-yl)piperidine-2,6-dione |
| 656 | 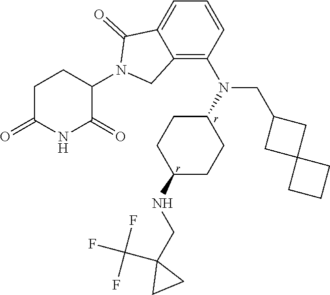 [SMILES: O=C1CCC(N2Cc3c(cccc3N(CC3CC4(CCC4)C3)[C@H]3CC[C@H](NCC4(C(F)(F)F)CC4)CC3)C2=O)C(=O)N1] | 3-(1-oxo-4-((spiro[3.3]heptan-2- ylmethyl)((1r,4r)-4-(((1- (trifluoromethyl)cyclopropyl)methyl) amino)cyclohexyl)amino)isoindolin-2- yl)piperidine-2,6-dione |
| 656A | 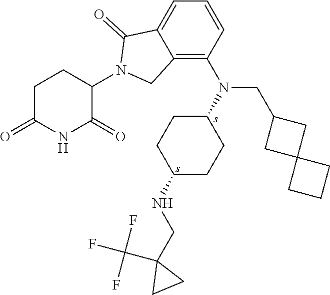 [SMILES: O=C1CCC(N2Cc3c(cccc3N(CC3CC4(CCC4)C3)[C@H]3CC[C@@H](NCC4(C(F)(F)F)CC4)CC3)C2=O)C(=O)N1] | 3-(1-oxo-4-((spiro[3.3]heptan-2- ylmethyl)((1s,4s)-4-(((1- (trifluoromethyl)cyclopropyl)methyl) amino)cyclohexyl)amino)isoindolin-2- yl)piperidine-2,6-dione |
| 657 | 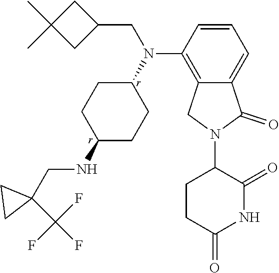 [SMILES: CC1(C)CC(CN(c2cccc3c2CN(C2CCC(=O)NC2=O)C3=O)[C@H]2CC[C@H](NCC3(C(F)(F)F)CC3)CC2)C1] | 3-(4-(((3,3- dimethylcyclobutyl)methyl)((1r,4r)-4- (((1- (trifluoromethyl)cyclopropyl)methyl) amino)cyclohexyl)amino)-1- oxoisoindolin-2-yl)piperidine-2,6-dione |
| 657A | 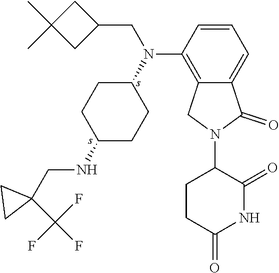 [SMILES: CC1(C)CC(CN(c2cccc3c2CN(C2CCC(=O)NC2=O)C3=O)[C@H]2CC[C@@H](NCC3(C(F)(F)F)CC3)CC2)C1] | 3-(4-(((3,3- dimethylcyclobutyl)methyl)((1s,4s)-4- (((1-(trifluoromethyl)cyclopropyl)methyl) amino)cyclohexyl)amino)-1- oxoisoindolin-2-yl)piperidine-2,6-dione |
| 658 | 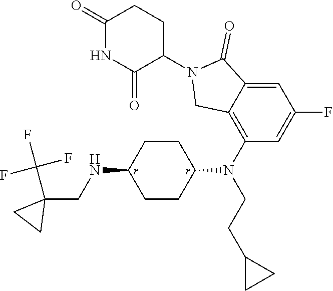 [SMILES: O=C1CCC(N2Cc3c(cc(F)cc3N(CCC3CC3)[C@H]3CC[C@H](NCC4(C(F)(F)F)CC4)CC3)C2=O)C(=O)N1] | 3-(4-((2-cyclopropylethyl)((1r,4r)-4- (((1- (trifluoromethyl)cyclopropyl)methyl) amino)cyclohexyl)amino)-6-fluoro-1- oxoisoindolin-2-yl)piperidine-2,6-dione |
| 658A | 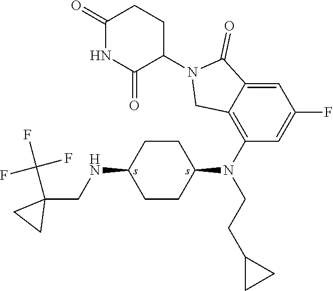 [SMILES: O=C1CCC(N2Cc3c(cc(F)cc3N(CCC3CC3)[C@H]3CC[C@@H](NCC4(C(F)(F)F)CC4)CC3)C2=O)C(=O)N1] | 3-(4-((2-cyclopropylethyl)((1s,4s)-4- (((1- (trifluoromethyl)cyclopropyl)methyl) amino)cyclohexyl)amino)-6-fluoro-1- oxoisoindolin-2-yl)piperidine-2,6-dione |
| 659 | 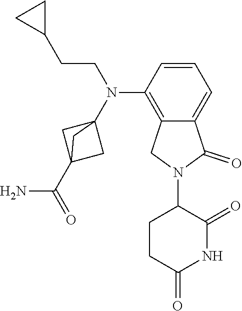 [SMILES: NC(=O)C12CC(N(CCC3CC3)c3cccc4c3CN(C3CCC(=O)NC3=O)C4=O)(C1)C2] | 3-((2-cyclopropylethyl)(2-(2,6- dioxopiperidin-3-yl)-1-oxoisoindolin- 4-yl)amino)bicyclo[1.1.1]pentane-1- carboxamide |
| 660 | 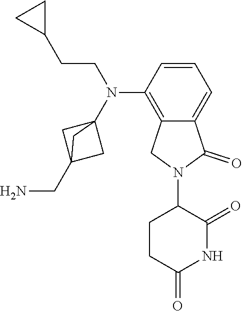 [SMILES: NCC12CC(N(CCC3CC3)c3cccc4c3CN(C3CCC(=O)NC3=O)C4=O)(C1)C2] | 3-(4-((3- (aminomethyl)bicyclo[1.1.1]pentan-1- yl)(2-cyclopropylethyl)amino)-1- oxoisoindolin-2-yl)piperidine-2,6- dione |
| 662 | 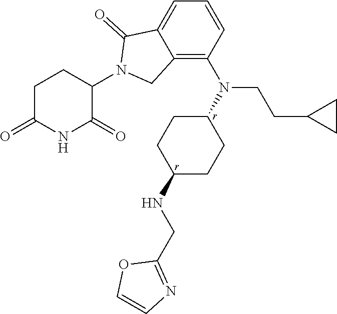 [SMILES: O=C1CCC(N2Cc3c(cccc3N(CCC3CC3)[C@H]3CC[C@H](NCc4ncco4)CC3)C2=O)C(=O)N1] | 3-(4-((2-cyclopropylethyl)((1r,4r)-4- ((oxazol-2- ylmethyl)amino)cyclohexyl)amino)-1- oxoisoindolin-2-yl)piperidine-2,6-dione |
| 663 | 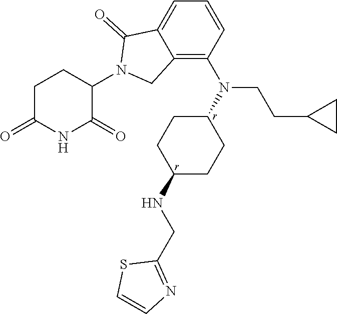 [SMILES: O=C1CCC(N2Cc3c(cccc3N(CCC3CC3)[C@H]3CC[C@H](NCc4nccs4)CC3)C2=O)C(=O)N1] | 3-(4-((2-cyclopropylethyl)((1r,4r)-4- ((thiazol- ylmethyl)amino)cyclohexyl)amino)-1- oxoisoindolin-2-yl)piperidine-2,6-dione |
| 664 | 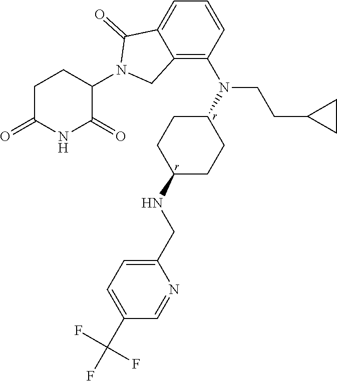 [SMILES: O=C1CCC(N2Cc3c(cccc3N(CCC3CC3)[C@H]3CC[C@H](NCc4ccc(C(F)(F)F)cn4)CC3)C2=O)C(=O)N1] | 3-(4-((2-cyclopropylethyl)((1r,4r)-4- (((5-(trifluoromethyl)pyridin-2- yl)methyl)amino)cyclohexyl)amino)-1- oxoisoindolin-2-yl)piperidine-2,6- dione |
| 665 | 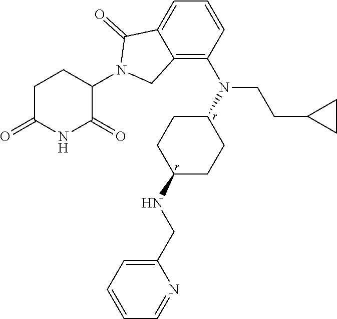 [SMILES: O=C1CCC(N2Cc3c(cccc3N(CCC3CC3)[C@H]3CC[C@H](NCc4ccccn4)CC3)C2=O)C(=O)N1] | 3-(4-((2-cyclopropylethyl)((1r,4r)-4- ((pyridin-2- ylmethyl)amino)cyclohexyl)amino)-1- oxoisoindolin-2-yl)piperidine-2,6-dione |
| 666 | 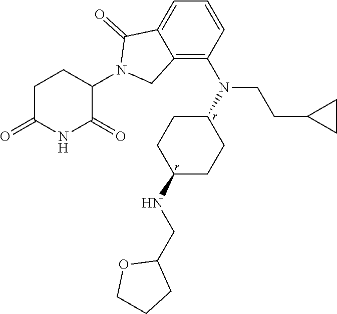 [SMILES: O=C1CCC(N2Cc3c(cccc3N(CCC3CC3)[C@H]3CC[C@H](NCC4CCCO4)CC3)C2=O)C(=O)N1] | 3-(4-((2-cyclopropylethyl)((1r,4r)-4- ((tetrahydrofuran-2- yl)methyl)amino)cyclohexyl)amino)-1- oxoisoindolin-2-yl)piperidine- 2,6-dione |
| 668 | 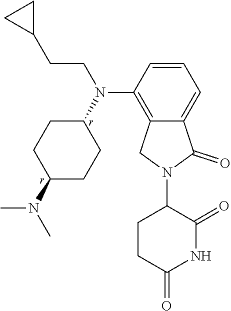 [SMILES: CN(C)[C@H]1CC[C@H](N(CCC2CC2)c2cccc3c2CN(C2CCC(=O)NC2=O)C3=O)CC1] | 3-(4-((2-cyclopropylethyl)((1r,4r)-4- (dimethylamino)cyclohexyl)amino)-1- oxoisoindolin-2-yl)piperidine-2,6-dione |
| 669 | 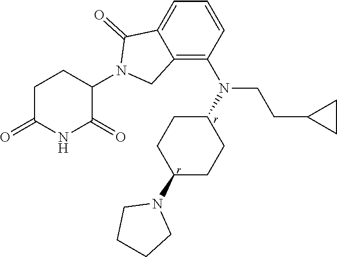 [SMILES: O=C1CCC(N2Cc3c(cccc3N(CCC3CC3)[C@H]3CC[C@H](N4CCCC4)CC3)C2=O)C(=O)N1] | 3-(4-((2-cyclopropylethyl)((1r,4r)-4- (pyrrolidin-1-yl)cyclohexyl)amino)-1- oxoisoindolin-2-yl)piperidine-2,6-dione |
| 669A | 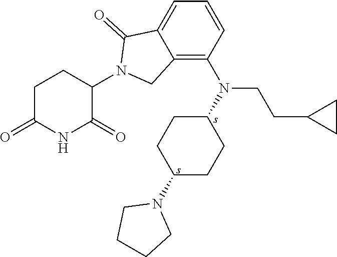 [SMILES: O=C1CCC(N2Cc3c(cccc3N(CCC3CC3)[C@H]3CC[C@@H](N4CCCC4)CC3)C2=O)C(=O)N1] | 3-(4-((2-cyclopropylethyl)((1s,4s)-4- (pyrrolidin-1-yl)cyclohexyl)amino)-1- oxoisoindolin-2-yl)piperidine-2,6-dione |
| 670 | 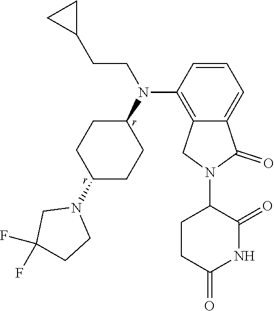 [SMILES: O=C1CCC(N2Cc3c(cccc3N(CCC3CC3)[C@H]3CC[C@H](N4CCC(F)(F)C4)CC3)C2=O)C(=O)N1] | 3-(4-((2-cyclopropylethyl)((1r,4r)-4- (3,3-difluoropyrrolidin-1- yl)cyclohexyl)amino)-1- oxoisoindolin-2-yl)piperidine-2,6-dione |
| 670A | 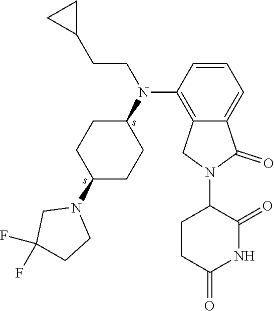 [SMILES: O=C1CCC(N2Cc3c(cccc3N(CCC3CC3)[C@H]3CC[C@@H](N4CCC(F)(F)C4)CC3)C2=O)C(=O)N1] | 3-(4-((2-cyclopropylethyl)((1s,4s)-4- (3,3-difluoropyrrolidin-1- yl)cyclohexyl)amino)-1- oxoisoindolin-2-yl)piperidine-2,6-dione |
| 671 | 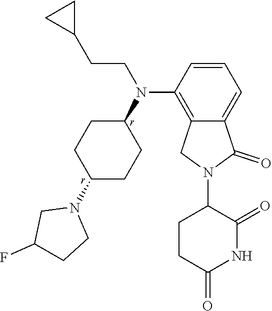 [SMILES: O=C1CCC(N2Cc3c(cccc3N(CCC3CC3)[C@H]3CC[C@H](N4CCC(F)C4)CC3)C2=O)C(=O)N1] | 3-(4-((2-cyclopropylethyl)((1r,4r)-4-(3- fluoropyrrolidin-1- yl)cyclohexyl)amino)-1- oxoisoindolin-2-yl)piperidine-2,6-dione |
| 671A | 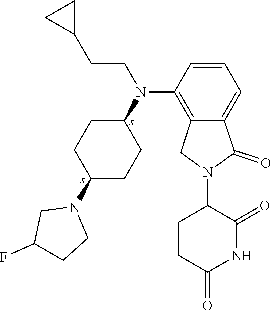 [SMILES: O=C1CCC(N2Cc3c(cccc3N(CCC3CC3)[C@H]3CC[C@@H](N4CCC(F)C4)CC3)C2=O)C(=O)N1] | 3-(4-((2-cyclopropylethyl)((1s,4s)-4- (3-fluoropyrrolidin-1- yl)cyclohexyl)amino)-1-oxoisoindolin- 2-yl)piperidine-2,6-dione |
| 672 | 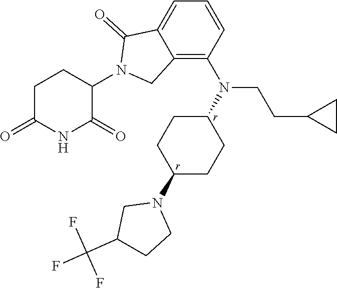 [SMILES: O=C1CCC(N2Cc3c(cccc3N(CCC3CC3)[C@H]3CC[C@H](N4CCC(C(F)(F)F)C4)CC3)C2=O)C(=O)N1] | 3-(4-((2-cyclopropylethyl)((1r,4r)-4-(3- (trifluoromethyl)pyrrolidin-1- yl)cyclohexyl)amino)-1- oxoisoindolin-2-yl)piperidine-2,6-dione |
| 672A | 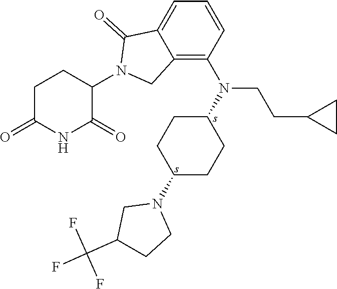 [SMILES: O=C1CCC(N2Cc3c(cccc3N(CCC3CC3)[C@H]3CC[C@@H](N4CCC(C(F)(F)F)C4)CC3)C2=O)C(=O)N1] | 3-(4-((2-cyclopropylethyl)((1s,4s)-4- (3-(trifluoromethyl)pyrrolidin-1- yl)cyclohexyl)amino)-1-oxoisoindolin- 2-yl)piperidine-2,6-dione |
| 673 | 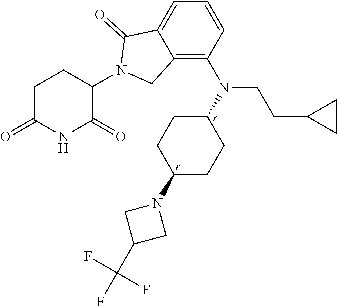 [SMILES: O=C1CCC(N2Cc3c(cccc3N(CCC3CC3)[C@H]3CC[C@H](N4CC(C(F)(F)F)C4)CC3)C2=O)C(=O)N1] | 3-(4-((2-cyclopropylethyl)((1r,4r)-4-(3- (trifluoromethyl)azetidin-1- yl)cyclohexyl)amino)-1- oxoisoindolin-2-yl)piperidine-2,6-dione |
| 673A | 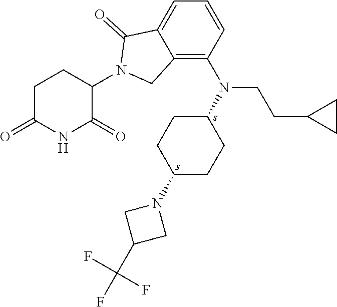 [SMILES: O=C1CCC(N2Cc3c(cccc3N(CCC3CC3)[C@H]3CC[C@@H](N4CC(C(F)(F)F)C4)CC3)C2=O)C(=O)N1] | 3-(4-((2-cyclopropylethyl)((1s,4s)-4- (3-(trifluoromethyl)azetidin-1- yl)cyclohexyl)amino)-1-oxoisoindolin- 2-yl)piperidine-2,6-dione |
| 674 | 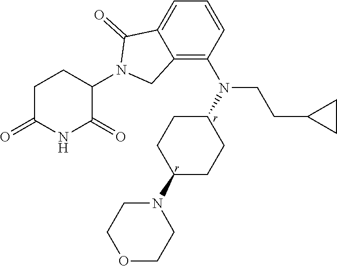 [SMILES: O=C1CCC(N2Cc3c(cccc3N(CCC3CC3)[C@H]3CC[C@H](N4CCOCC4)CC3)C2=O)C(=O)N1] | 3-(4-((2-cyclopropylethyl)((1r,4r)-4- morpholinocyclohexyl)amino)-1- oxoisoindolin-2-yl)piperidine-2,6- dione |
| 674A | 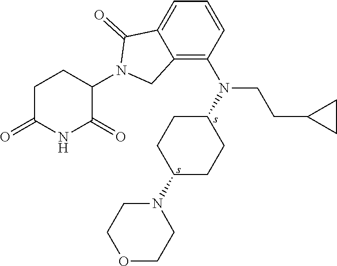 [SMILES: O=C1CCC(N2Cc3c(cccc3N(CCC3CC3)[C@H]3CC[C@@H](N4CCOCC4)CC3)C2=O)C(=O)N1] | 3-(4-((2-cyclopropylethyl)((1s,4s)-4- morpholinocyclohexyl)amino)-1- oxoisoindolin-2-yl)piperidine-2,6- dione |
| 675 | 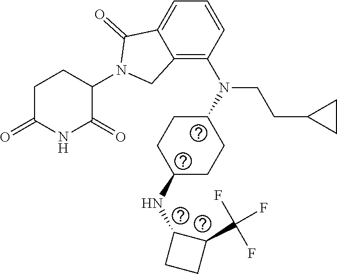 [SMILES: O=C1CCC(N2Cc3c(cccc3N(CCC3CC3)[C@H]3CC[C@H](N[C@H]4CC[C@@H]4C(F)(F)F)CC3)C2=O)C(=O)N1] | 3-(4-((2-cyclopropylethyl)((1S,4s)-4- (((1S,2S)-2- (trifluoromethyl)cyclobutyl)amino) cyclohexyl)amino-1- oxoisoindolin-2-yl)piperidine-2,6-dione |
| 675A | 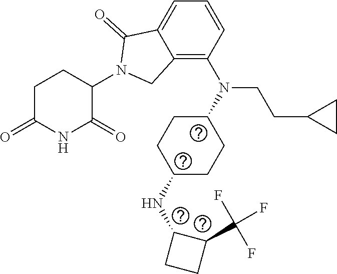 [SMILES: O=C1CCC(N2Cc3c(cccc3N(CCC3CC3)[C@H]3CC[C@@H](N[C@H]4CC[C@@H]4C(F)(F)F)CC3)C2=O)C(=O)N1] | 3-(4-((2-cyclopropylethyl)((1R,4s)-4- (((1S,2S)-2- (trifluoromethyl)cyclobutyl)amino) cyclohexyl)amino)-1-oxoisoindolin-2- yl)piperidine-2,6-dione |
| 678 | 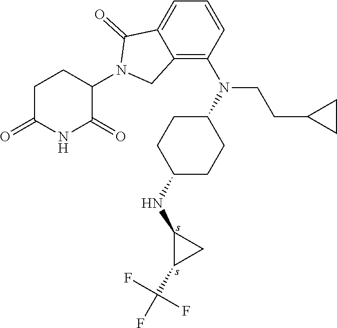 [SMILES: O=C1CCC(N2Cc3c(cccc3N(CCC3CC3)[C@H]3CC[C@@H](N[C@H]4C[C@@H]4C(F)(F)F)CC3)C2=O)C(=O)N1] | 3-(4-((2-cyclopropylethyl)(4-(((1S,2S)- 2- (trifluoromethyl)cyclopropyl)amino) cyclohexyl)amino)-1- oxoisoindolin-2-yl)piperidine-2,6-dione |
| 679 | 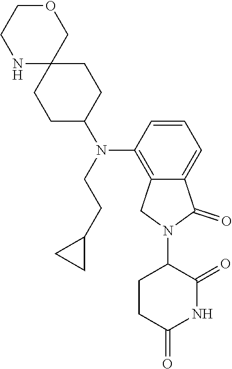 [SMILES: O=C1CCC(N2Cc3c(cccc3N(CCC3CC3)C3CCC4(CC3)COCCN4)C2=O)C(=O)N1] | 3-(4-((2-cyclopropylethyl)(4-oxa-1- azaspiro[5.5]undecan-9-yl)amino)-1- oxoisoindolin-2-yl)piperidine-2,6-dione |
| 680 | 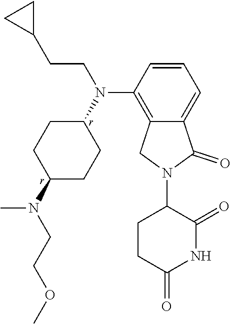 [SMILES: COCCN(C)[C@H]1CC[C@H](N(CCC2CC2)c2cccc3c2CN(C2CCC(=O)NC2=O)C3=O)CC1] | 3-(4-((2-cyclopropylethyl)((1r,4r)-4- ((2- methoxyethyl)(methyl)amino)cyclohexyl) amino)-1- oxoisoindolin-2-yl)piperidine-2,6-dione |
| 680A | 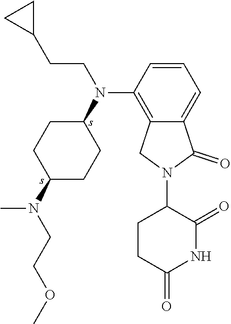 [SMILES: COCCN(C)[C@H]1CC[C@@H](N(CCC2CC2)c2cccc3c2CN(C2CCC(=O)NC2=O)C3=O)CC1] | 3-(4-((2-cyclopropylethyl)((1s,4s)-4- ((2- methoxyethyl)(methyl)amino)cyclohexyl) amino)-1- oxoisoindolin-2-yl)piperidine-2,6-dione |
| 681 | 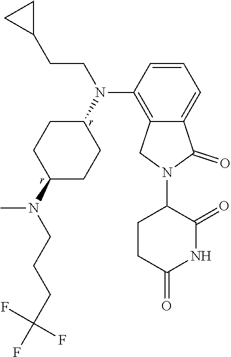 [SMILES: CN(CCCC(F)(F)F)[C@H]1CC[C@H](N(CCC2CC2)c2cccc3c2CN(C2CCC(=O)NC2=O)C3=O)CC1] | 3-(4-((2-cyclopropylethyl)((1r,4r)-4- (methyl(4,4,4- trifluorobutyl)amino)cyclohexyl) amino)-1- oxoisoindolin-2-yl)piperidine-2,6-dione |
| 681A | 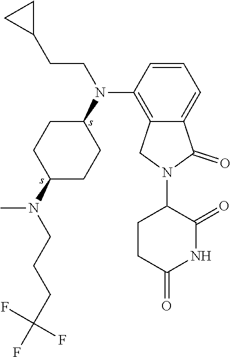 [SMILES: CN(CCCC(F)(F)F)[C@H]1CC[C@@H](N(CCC2CC2)c2cccc3c2CN(C2CCC(=O)NC2=O)C3=O)CC1] | 3-(4-((2-cyclopropylethyl)((1s,4s)-4- (methyl(4,4,4- trifluorobutyl)amino)cyclohexyl)amino)- 1-oxoisoindolin-2-yl)piperidine-2,6-dione |
| 682 | 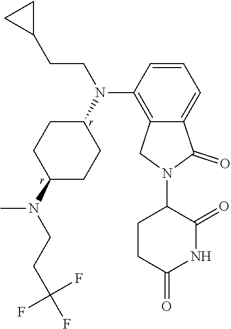 [SMILES: CN(CCC(F)(F)F)[C@H]1CC[C@H](N(CCC2CC2)c2cccc3c2CN(C2CCC(=O)NC2=O)C3=O)CC1] | 3-(4-((2-cyclopropylethyl)((1r,4r)-4- (methyl(3,3,3- trifluoropropyl)amino)cyclohexyl) amino)-1-oxoisoindolin-2-yl)piperidine- 2,6-dione |
| 682A | 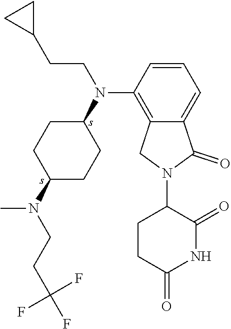 [SMILES: CN(CCC(F)(F)F)[C@H]1CC[C@@H](N(CCC2CC2)c2cccc3c2CN(C2CCC(=O)NC2=O)C3=O)CC1] | 3-(4-((2-cyclopropylethyl)((1s,4s)-4- (methyl(3,3,3-trifluoropropyl)amino)cyclohexyl) amino)-1-oxoisoindolin-2-yl)piperidine- 2,6-dione |
| 683 | 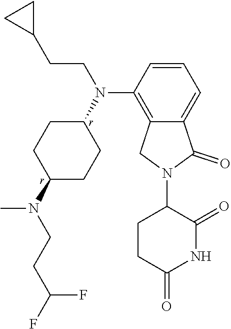 [SMILES: CN(CCC(F)F)[C@H]1CC[C@H](N(CCC2CC2)c2cccc3c2CN(C2CCC(=O)NC2=O)C3=O)CC1] | 3-(4-((2-cyclopropylethyl)((1r,4r)-4- ((3,3-difluoropropyl)(methyl)amino) cyclohexyl)amino)-1- oxoisoindolin-2-yl)piperidine-2,6-dione |
| 683A | 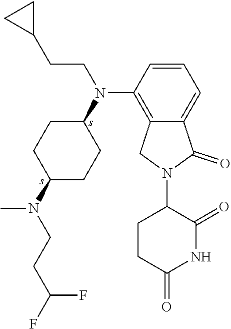 [SMILES: CN(CCC(F)F)[C@H]1CC[C@@H](N(CCC2CC2)c2cccc3c2CN(C2CCC(=O)NC2=O)C3=O)CC1] | 3-(4-((2-cyclopropylethyl)((1s,4s)-4- ((3,3-difluoropropyl)(methyl)amino) cyclohexyl)amino)-1-oxoisoindolin- 2-yl)piperidine-2,6-dione |
| 703 | 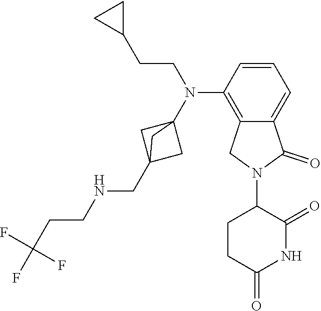 [SMILES: O=C1CCC(N2Cc3c(cccc3N(CCC3CC3)C34CC(CNCCC(F)(F)F)(C3)C4)C2=O)C(=O)N1] | 3-(4-((2-cyclopropylethyl)(3-(((3,3,3- trifluoropropyl)amino)methyl) bicyclo[1.1.1]pentan-1-yl)amino)-1- oxoisoindolin-2-yl)piperidine-2,6- dione |
| 708 | 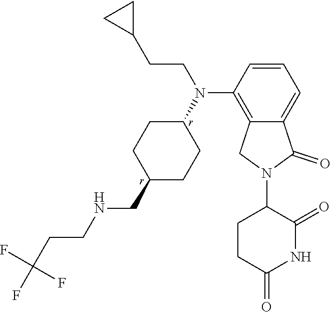 [SMILES: O=C1CCC(N2Cc3c(cccc3N(CCC3CC3)[C@H]3CC[C@H](CNCCC(F)(F)F)CC3)C2=O)C(=O)N1] | 3-(4-((2-cyclopropylethyl)((1r,4r)-4- (((3,3,3- trifluoropropyl)amino)methyl) cyclohexyl)amino)-1- oxoisoindolin-2-yl)piperidine-2,6-dione |
| 708A | 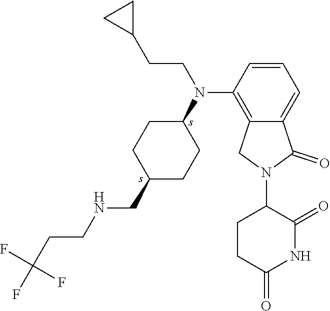 [SMILES: O=C1CCC(N2Cc3c(cccc3N(CCC3CC3)[C@H]3CC[C@@H](CNCCC(F)(F)F)CC3)C2=O)C(=O)N1] | 3-(4-((2-cyclopropylethyl)((1s,4s)-4- (((3,3,3- trifluoropropyl)amino)methyl) cyclohexyl)amino)-1-oxoisoindolin-2- yl)piperidine-2,6-dione |
| 711 | 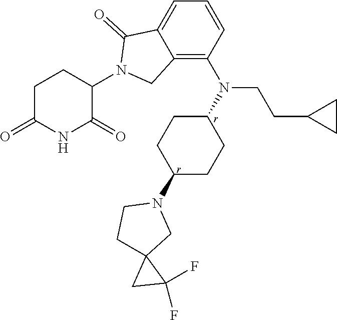 [SMILES: O=C1CCC(N2Cc3c(cccc3N(CCC3CC3)[C@H]3CC[C@H](N4CCC5(C4)CC5(F)F)CC3)C2=O)C(=O)N1] | 3-(4-((2-cyclopropylethyl)((1r,4r)-4- (1,1-difluoro-5-azaspiro[2.4]heptan-5- yl)cyclohexyl)amino)-1- oxoisoindolin-2-yl)piperidine-2,6-dione |
| 711A | 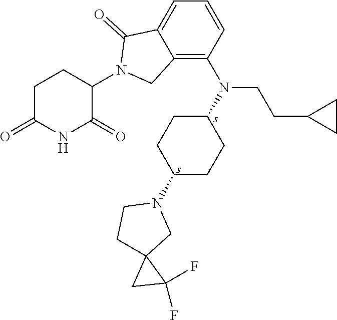 [SMILES: O=C1CCC(N2Cc3c(cccc3N(CCC3CC3)[C@H]3CC[C@@H](N4CCC5(C4)CC5(F)F)CC3)C2=O)C(=O)N1] | 3-(4-((2-cyclopropylethyl)((1s,4s)-4- (1,1-difluoro-5-azaspiro[2.4]heptan-5- yl)cyclohexyl)amino)-1- oxoisoindolin-2-yl)piperidine-2,6-dione |
| 712 | 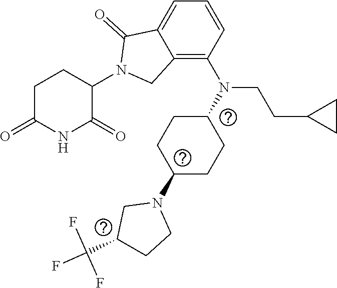 [SMILES: O=C1CCC(N2Cc3c(cccc3N(CCC3CC3)[C@H]3CC[C@H](N4CC[C@H](C(F)(F)F)C4)CC3)C2=O)C(=O)N1] | 3-(4-((2-cyclopropylethyl)((1S,4r)-4- ((S)-3-(trifluoromethyl)pyrrolidin-1- yl)cyclohexyl)amino)-1- oxoisoindolin-2-yl)piperidine-2,6-dione |
| 712A | 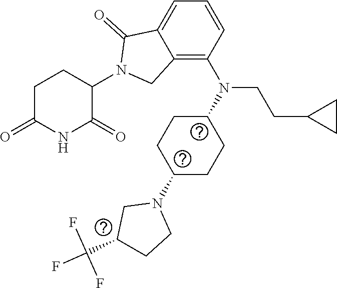 [SMILES: O=C1CCC(N2Cc3c(cccc3N(CCC3CC3)[C@H]3CC[C@@H](N4CC[C@H](C(F)(F)F)C4)CC3)C2=O)C(=O)N1] | 3-(4-((2-cyclopropylethyl)((1R,4s)-4- ((S)-3-(trifluoromethyl)pyrrolidin-1- yl)cyclohexyl)amino)-1- oxoisoindolin-2-yl)piperidine-2,6-dione |
| 713 | 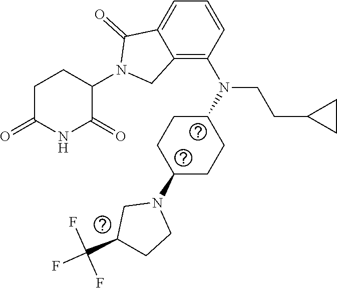 [SMILES: O=C1CCC(N2Cc3c(cccc3N(CCC3CC3)[C@H]3CC[C@H](N4CC[C@@H](C(F)(F)F)C4)CC3)C2=O)C(=O)N1] | 3-(4-((2-cyclopropylethyl)((1R,4r)-4- ((R)-3-(trifluoromethyl)pyrrolidin-1- yl)cyclohexyl)amino)-1- oxoisoindolin-2-yl)piperidine-2,6-dione |
| 713A | 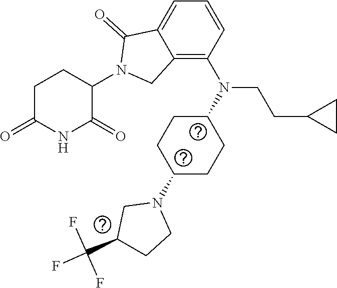 [SMILES: O=C1CCC(N2Cc3c(cccc3N(CCC3CC3)[C@H]3CC[C@@H](N4CC[C@@H](C(F)(F)F)C4)CC3)C2=O)C(=O)N1] | 3-(4-((2-cyclopropylethyl)((1S,4s)-4- ((R)-3-(trifluoromethyl)pyrrolidin-1- yl)cyclohexyl)amino)-1- oxoisoindolin-2-yl)piperidine-2,6-dione |
| 714 | 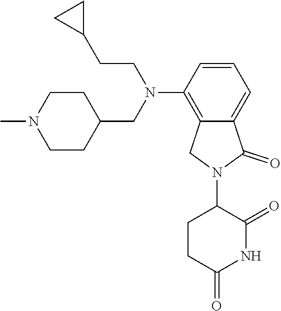 [SMILES: CN1CCC(CN(CCC2CC2)c2cccc3c2CN(C2CCC(=O)NC2=O)C3=O)CC1] | 3-(4-((2-cyclopropylethyl)((1- methylpiperidin-4-yl)methyl)amino)-1- oxoisoindolin-2-yl)piperidine-2,6-dione |
| 715 | 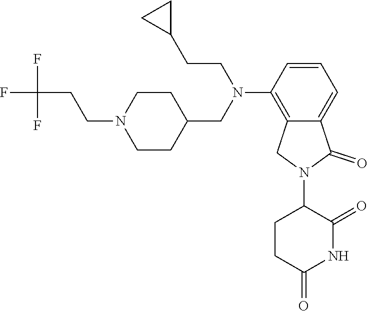 [SMILES: O=C1CCC(N2Cc3c(cccc3N(CCC3CC3)CC3CCN(CCC(F)(F)F)CC3)C2=O)C(=O)N1] | 3-(4-((2-cyclopropylethyl)((1-(3,3,3- trifluoropropyl)piperidin-4- yl)methyl)amino)-1- oxoisoindolin-2-yl)piperidine-2,6-dione |
| 716 | 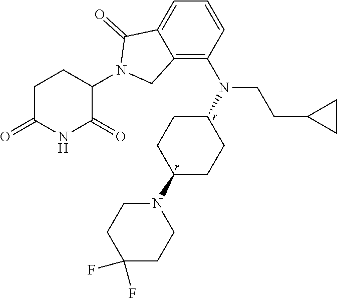 [SMILES: O=C1CCC(N2Cc3c(cccc3N(CCC3CC3)[C@H]3CC[C@H](N4CCC(F)(F)CC4)CC3)C2=O)C(=O)N1] | 3-(4-((2-cyclopropylethyl)((1r,4r)-4- (4,4-difluoropiperidin-1- yl)cyclohexyl)amino)-1- oxoisoindolin-2-yl)piperidine-2,6-dione |
| 716A | 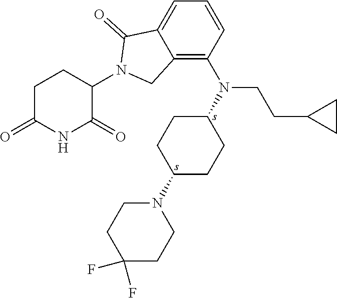 [SMILES: O=C1CCC(N2Cc3c(cccc3N(CCC3CC3)[C@H]3CC[C@@H](N4CCC(F)(F)CC4)CC3)C2=O)C(=O)N1] | 3-(4-((2-cyclopropylethyl)((1s,4s)-4- (4,4-difluoropiperidin-1- yl)cyclohexyl)amino)-1- oxoisoindolin-2-yl)piperidine-2,6-dione |

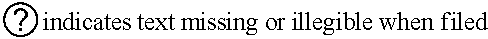

In some embodiment, the compound of the present disclosure is for use in degrading and/or reducing casein kinase 1 alpha (CK1α).

In some embodiment, the compound of the present disclosure is for use in inhibiting casein kinase 1 alpha (CK1α) activity.

In some embodiment, the compound of the present disclosure is for use in preventing or treating blood cancer.

In some embodiment, the present disclosure provides a pharmaceutical composition comprising: the compound of the present disclosure (represented by Formula (I), (I-a), (I-b) or (I-c)), or the pharmaceutically acceptable salt, enantiomer, stereoisomer or prodrug thereof, and optionally a pharmaceutically acceptable excipient or carrier.

The term “pharmaceutically acceptable” describes a material that is not biologically or otherwise undesirable, i.e., without causing an unacceptable level of undesirable biological effects or interacting in a deleterious manner.

The term “pharmaceutically acceptable carrier” is used herein to refer to a carrier that is useful in preparing a pharmaceutical composition that is generally safe, non-toxic and neither biologically nor otherwise-undesirable, and is acceptable for veterinary use as well as human pharmaceutical use. A “pharmaceutically acceptable carrier” as used in the specification and claims can include both one and more than one such carrier. By “pharmaceutically acceptable” it is meant the carrier must be compatible with the other ingredients of the formulation and not deleterious to the recipient thereof.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The compounds represented by Formula (I), or a pharmaceutically acceptable salt, enantiomer, stereoisomer or prodrug thereof degrade casein kinase 1 alpha (CK1α).

In some embodiment, the pharmaceutical composition is for use in degrading and/or reducing casein kinase 1 alpha (CK1α).

In some embodiment, the pharmaceutical composition is for use in inhibiting casein kinase 1 alpha (CK1α) activity.

In some embodiment, the pharmaceutical composition is for use in preventing or treating blood cancer.

In some embodiment, the blood cancer is leukemia, lymphoma, or myeloma.

The present disclosure also provides a method of degrading and/or reducing casein kinase 1 alpha (CK1α) in a subject in need thereof, the method comprising administering to the subject an effective amount of the pharmaceutical composition of the present disclosure.

The present disclosure also provides a method of inhibiting casein kinase 1 alpha (CK1α) activity in a subject in need thereof, the method comprising administering to the subject an effective amount of the pharmaceutical composition of the present disclosure.

The present disclosure also provides a method of preventing or treating blood cancer in a subject in need thereof, the method comprising administering to the subject an effective amount of the pharmaceutical composition of the present disclosure.

As used herein, the term “blood cancer” refers to cancer that begins in blood-forming tissue, such as the bone marrow, or in the cells of the immune system. In some embodiment, the “blood cancer” can include any cancer involving uncontrolled proliferation of blood cells, in particular white blood cells.

In some embodiment, the blood cancer is leukemia, lymphoma (Hodgkin and non-Hodgkin lymphomas), or myeloma.

In some embodiment, the leukemia is selected from the group consisting of acute myeloid leukemia (AML), acute lymphoblastic leukemia or acute lymphocytic leukemia (ALL), T-cell acute lymphoblastic leukemia (T-ALL), B-cell acute lymphoblastic leukemia (B-ALL), chronic myeloid leukemia (CML), chronic lymphocytic leukemia (CLL), hairy cell leukemia (HCL), T-cell prolymphocytic leukemia (T-PLL), Large granular lymphocytic leukemia, adult T-cell leukemia, chronic eosinophilic leukemia (CEL) and myelodysplastic syndrome (MDS).

In some embodiment, the lymphoma is selected from the group consisting of Hodgkin's lymphoma, diffuse large B-cell lymphoma (DLBCL), follicular lymphoma, small lymphocytic lymphoma (SLL), mantle cell lymphoma (MCL), marginal zone lymphomas, Burkitt lymphoma, lymphoplasmacytic lymphoma, primary central nervous system (CNS) lymphoma and peripheral T-cell lymphoma.

In some embodiment, the myeloma is selected from the group consisting of multiple myeloma, light chain myeloma, non-secretory myeloma, solitary plasmacytoma, extramedullary plasmacytoma, monoclonal gammopathy of undetermined significance (MGUS), smoldering multiple myeloma (SMM), immunoglobulin D (IgD) myeloma and immunoglobulin E (IgE) myeloma.

In some embodiments, the compounds of the present disclosure may bind to cereblon (CRBN), altering the specificity of the complex to induce the ubiquitination and degradation of CK1α, transcription factors essential for proliferative disorders such as multiple myeloma growth.

In some embodiments, the present disclosure provides compounds and compositions comprising an E3 ligase binding moiety that can bind to an E3 ligase (e.g., CRBN) and target protein binding moiety that can bind to a target protein, which results in the ubiquitination of a target protein and leads to degradation of the target protein by the proteasome.

As used herein, the term “degrade” or “degradation” as used herein means the degradation of a target protein mediated by an E3 ligase, for example, CRBN, resulting in a reduction of the protein levels. In one embodiment, the target protein can be Casein Kinase 1 (CK1, e.g., CK1α, CK1β1, CK1γ1, CK1γ2, CK1γ3, CK1δ, CK1ε) protein. In certain embodiments, the target protein preferably can be CK1α.

The term “CK1α” as used herein refers to Casein Kinase 1α, a kinase in humans that is encoded by the CSNK1A1 gene. CK1α has been shown to play a critical role in the biology of AML (Jaras M et al, J Exp Med. 2014; 211(4):605-612).

As used herein, the term “effective amount” refers to that amount of an active agent being administered sufficient to degrade CK1α and/or inhibit CK1α activity. In certain embodiments, the term “effective amount” refers to that amount of an active agent being administered sufficient to treat a CK1α related disease, preferably blood cancer.

As used herein, the term “treat,” “treatment,” or “treating,” refers to administering a compound or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

As used herein, the term “preventing” refers to a slowing of the disease or of the onset of the disease or the symptoms thereof. Preventing a disease or disorder can include stopping the onset of the disease or symptoms thereof.

The pharmaceutical compositions described herein can be administered to a human patient per se, or in pharmaceutical compositions where they are mixed with other active ingredients, as in combination therapy, or excipients, or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound, salt and/or composition exist in the art including, but not limited to, oral, rectal, pulmonary, topical, aerosol, injection, infusion and parenteral delivery, including intramuscular, subcutaneous, intravenous, intramedullary injections, intrathecal, direct intraventricular, intraperitoneal, intranasal and intraocular injections. In some embodiments, a compound described herein, including a compound of Formula (I) or a pharmaceutically acceptable salt, enantiomer, stereoisomer or prodrug thereof, can be administered orally.

One may also administer the compound, salt and/or composition in a local rather than systemic manner, for example, via injection or implantation of the compound directly into the affected area, often in a depot or sustained release formulation. Furthermore, one may administer the compound in a targeted drug delivery system, for example, in a liposome coated with a tissue-specific antibody. The liposomes will be targeted to and taken up selectively by the organ. For example, intranasal or pulmonary delivery to target a respiratory disease or condition may be desirable.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound and/or salt described herein formulated in a compatible pharmaceutical excipient may also be prepared, placed in an appropriate container, and labeled for treatment of an indicated condition.

The compounds, salt and/or pharmaceutical composition can be provided to an administering physician or other health care professional in the form of a kit. The kit is a package which houses a container which contains the compound(s) in a suitable pharmaceutical composition, and instructions for administering the pharmaceutical composition to a subject. The kit can optionally also contain one or more additional therapeutic agents. The kit can also contain separate doses of a compound(s) or pharmaceutical composition for serial or sequential administration. The kit can optionally contain one or more diagnostic tools and instructions for use. The kit can contain suitable delivery devices, for example, syringes, and the like, along with instructions for administering the compound(s) and any other therapeutic agent. The kit can optionally contain instructions for storage, reconstitution (if applicable), and administration of any or all therapeutic agents included. The kits can include a plurality of containers reflecting the number of administrations to be given to a subject.

As will be readily apparent to one skilled in the art, the useful in vivo dosage to be administered and the particular mode of administration will vary depending upon the age, weight, the severity of the affliction, and mammalian species treated, the particular compounds employed, and the specific use for which these compounds are employed. The determination of effective dosage levels, that is the dosage levels necessary to achieve the desired result, can be accomplished by one skilled in the art using routine methods, for example, human clinical trials and in vitro studies.

The dosage may range broadly, depending upon the desired effects and the therapeutic indication. Alternatively dosages may be based and calculated upon the surface area of the patient, as understood by those of skill in the art. Although the exact dosage will be determined on a drug-by-drug basis, in most cases, some generalizations regarding the dosage can be made. The daily dosage regimen for an adult human patient may be, for example, an oral dose of between about 0.01 mg and 3,000 mg of each active ingredient, preferably between about 1 mg and 700 mg, e.g., about 5 to 200 mg. The dosage may be a single one or a series of two or more given in the course of one or more days, as is needed by the subject. In some embodiments, the compounds will be administered for a period of continuous therapy, for example for a week or more, or for months or years.

In instances where human dosages for compounds have been established for at least some condition, those same dosages may be used, or dosages that are between about 0.1% and 500%, more preferably between about 25% and 250% of the established human dosage. Where no human dosage is established, as will be the case for newly-discovered pharmaceutical compositions, a suitable human dosage can be inferred from ED₅₀ or Id₅₀ values, or other appropriate values derived from in vitro or in vivo studies, as qualified by toxicity studies and efficacy studies in animals.

In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for about 10 to 90% of the time, preferably between about 30 to 90% and most preferably between about 50 to 90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Compounds disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

### Examples

### I. Synthesis of Compounds

### Hydrochloride Salt Form of Compound 1

### 3-(4-((4-Aminobutyl)amino)-1-oxoisoindolin-2-yl)piperidine-2,6-dione hydrochloride

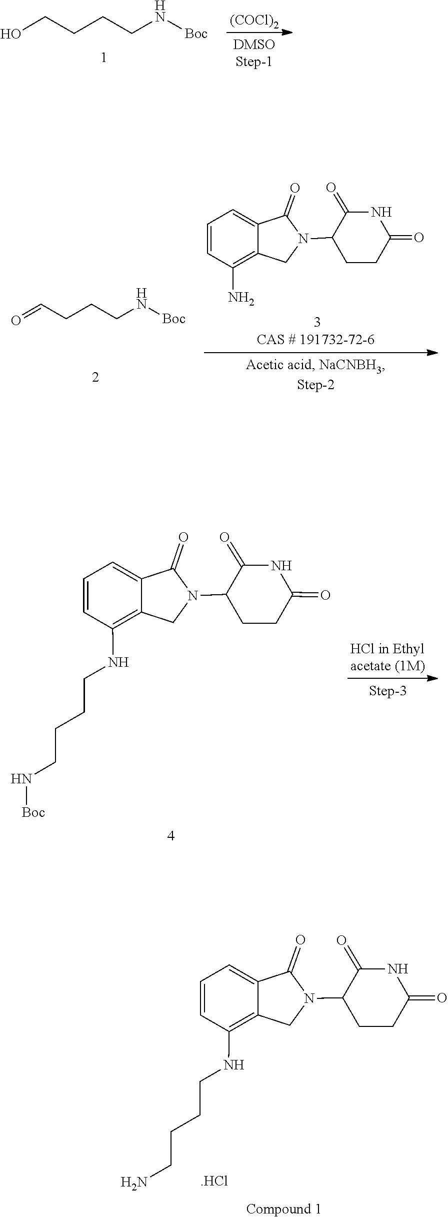

### Step-1: tert-Butyl (4-oxobutyl) carbamate

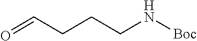 [SMILES: CC(C)(C)OC(=O)NCCCC=O]

A solution of oxalyl chloride (5.4 mL, 63.49 mmol, 1.2 equiv) in DCM (50 mL) was added dropwise to a solution of DMSO (10 mL, 1 vol) in DCM (100 mL, 10 vol) at −78° C. over a period of 15 minutes under nitrogen atmosphere and stirred for 15 minutes at the same temperature. To this a solution of tert-Butyl (4-hydroxybutyl)carbamate 1 (10 g, 52.91 mmol, 1 equiv) in DCM (50 mL) was added dropwise over a period of 15 minutes. After stirring the reaction mixture for 30 minutes at −78° C., was added N, N-diisopropylethylamine (29.2 mL, 158.7 mmol, 3 equiv) in drops at −78° C. Once addition was complete, the reaction mixture was slowly warmed to 0° C. and stirred for 30 minutes. Upon completion of the reaction (as confirmed by TLC analysis, 2: 8/EtOAc: pet ether, R_{f}˜0.6, KMnO₄), the reaction mixture was diluted with DCM (200 mL) and washed with 10% aq citric acid solution (1×100 mL), water (1×200 mL) and brine (1×200 mL), dried over Na₂SO₄, filtered and concentrated under vacuum below 30° C. to afford tert-butyl (4-oxobutyl) carbamate (10 g, crude) as pale yellow liquid which was used further without purification.

¹H-NMR (400 MHz, DMSO-d₆): δ 9.66 (s, 1H), 6.84 (s, 1H), 2.89-2.93 (m, 2H), 2.42-2.44 (m, 2H), 1.60-1.64 (m, 2H), 1.38 (s, 9H).

### Step-2: tert-Butyl(4-((2-(2,6-dioxopiperidin-3-yl)-1-oxoisoindolin-4-yl)amino)butyl)carbamate

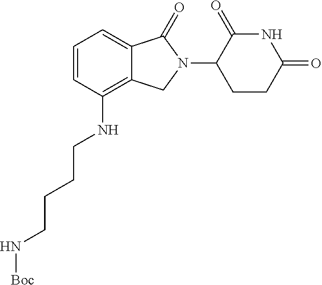 [SMILES: CC(C)(C)OC(=O)NCCCCNc1cccc2c1CN(C1CCC(=O)NC1=O)C2=O]

A mixture of tert-Butyl (4-oxobutyl) carbamate 2 (9.0 g, 0.0484 mol, 1.8 equiv) and Lenolidamide 3 (CAS #191732-72-6, 7.0 g, 0.0269 mol, 1 equiv) were dissolved in a mixture of 1,2-Dichloroethane (70 mL, 10 vol) and DMF (70 mL, 10 vol) under nitrogen atmosphere. To this was added acetic acid (4.8 mL, 0.0807 mol, 3.0 equiv) at room temperature. The resulted reaction mixture was stirred for 2 hours at room temperature. At this stage, NaCNBH₃ (6.6 g, 0.1076 mol, 4 equiv) was added in portions at 0° C. and stirred for 16 hours at room temperature. Upon completion of the reaction (as confirmed by TLC analysis, 8: 2/EtOAc: pet ether, R_{f}˜0.6, and LCMS), the reaction mixture was diluted with DCM (200 mL) and washed water (3×200 mL) and brine (200 mL), dried over Na₂SO₄, filtered and concentrated. The resulted product was triturated with hexane (50 mL), filtered and dried under suction to afford tert-Butyl(4-((2-(2,6-dioxopiperidin-3-yl)-1-oxoisoindolin-4-yl)amino)butyl)carbamate 4 (8 g, 69%) as white solid.

¹H-NMR (400 MHz, DMSO-d₆): δ 11.08 (s, 1H), 7.28 (t, J=7.60 Hz, 1H), 6.93 (d, J=7.20 Hz, 1H), 6.82 (t, J=5.60 Hz, 1H), 6.75 (d, J=8.00 Hz, 1H), 5.58 (t, J=5.60 Hz, 1H), 5.10-5.14 (m, 1H), 4.10-4.25 (m, 2H), 3.11-3.14 (m, 2H), 2.90-2.98 (m, 3H), 2.60-2.64 (m, 1H), 2.28-2.32 (m, 1H), 2.04-2.06 (m, 1H), 1.46-1.58 (m, 4H), 1.37 (s, 9H). LC-MS: 429.2 (M-H), Rt (min): 2.552, Area %—97.64.

### Step-3: 3-(4-((4-Aminobutyl)amino)-1-oxoisoindolin-2-yl)piperidine-2,6-dione hydrochloride

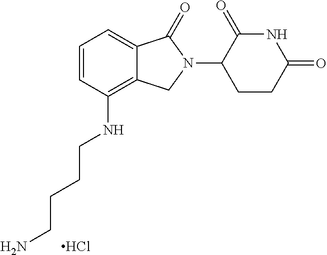 [SMILES: NCCCCNc1cccc2c1CN(C1CCC(=O)NC1=O)C2=O ; Cl]

To an ice cold suspension of tert-Butyl (4-((2-(2,6-dioxopiperidin-3-yl)-1-oxoisoindolin-4-yl)amino)butyl)carbamate 4 (800 mg, 1.86 mmol, 1 equiv) in DCM (10 mL) was added HCl in EtOAc (1 M, 8 mL) and stirred for 4 hours at room temperature. The progress of the reaction was monitored by LCMS. The reaction mixture was concentrated and lyophilized to obtain 3-(4-((4-Aminobutyl)amino)-1-oxoisoindolin-2-yl)piperidine-2,6-dione hydrochloride (530 mg, 79%) as yellow solid.

¹H-NMR (400 MHz, DMSO-d₆): δ 11.02 (s, 1H), 8.07 (s, 3H), 7.34 (t, J=7.60 Hz, 1H), 7.05 (d, J=7.60 Hz, 1H), 6.92 (d, J=8.00 Hz, 1H), 5.10-5.15 (m, 1H), 4.19-4.37 (m, 2H), 3.19-3.20 (m, 2H), 2.90-2.95 (m, 1H), 2.80-2.89 (m, 2H), 2.51-2.52 (m, 1H), 2.29-2.33 (m, 1H), 2.03-2.06 (m, 1H), 1.66-1.67 (m, 4H). ¹³C-NMR (100 MHz, DMSO-d₆): δ 173.34, 171.52, 168.67, 140.34, 133.14, 129.87, 117.06, 114.76, 52.08, 46.58, 44.46, 31.65, 24.90, 23.23. LCMS: 331.3 (M+H). Method: Mobile Phase A: 0.1% Formic Acid in H₂O. Mobile Phase B: Acetonitrile. Flow rate: 1.5 mL/min. COLUMN: Atlantis dC18 (50×4.6) mm, 5μ. Rt (min): 1.638; Area %—99.32. HPLC Method: Mobile Phase A: 0.1% TFA in H₂O. Mobile Phase B: Acetonitrile. Flow rate: 1.0 mL/min. COLUMN: Atlantis dC18 (250×4.6) mm, 5 μm. Rt (min): 7.403; Area %—98.88.

### Hydrochloride Salt Form of Compound 2

### 3-(4-((4-Aminobutyl)(methyl)amino)-1-oxoisoindolin-2-yl)piperidine-2,6-dione hydrochloride

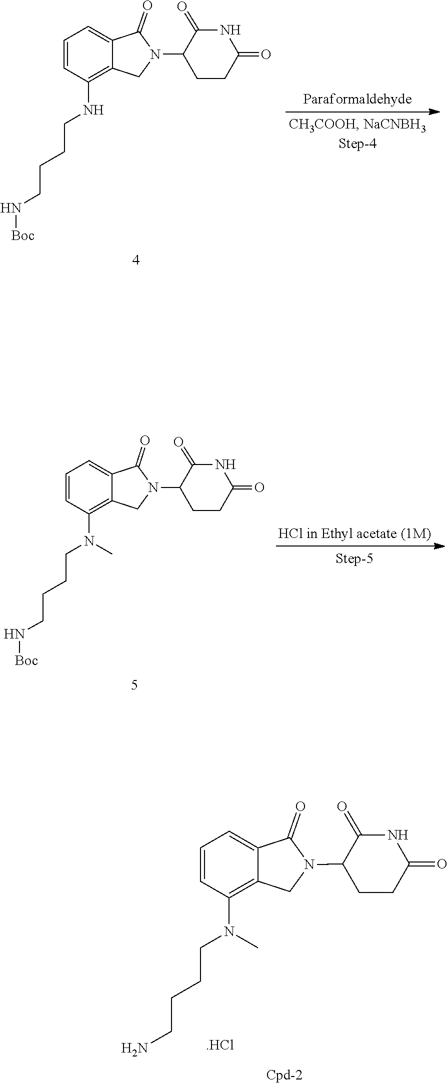 [SMILES: CC(C)(C)OC(=O)NCCCCNc1cccc2c1CN(C1CCC(=O)NC1=O)C2=O ; CN(CCCCNC(=O)OC(C)(C)C)c1cccc2c1CN(C1CCC(=O)NC1=O)C2=O ; CN(CCCCN)c1cccc2c1CN(C1CCC(=O)NC1=O)C2=O ; ".HCl"]

### Step-1: tert-Butyl(4-((2-(2,6-dioxopiperidin-3-yl)-1-oxoisoindolin-4-yl)(methyl)amino)butyl)carbamate

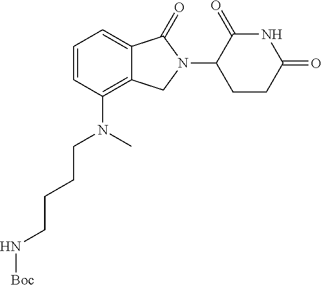 [SMILES: CN(CCCCNC(=O)OC(C)(C)C)c1cccc2c1CN(C1CCC(=O)NC1=O)C2=O]

Tert-Butyl(4-((2-(2,6-dioxopiperidin-3-yl)-1-oxoisoindolin-4-yl)amino)butyl)carbamate 4 (2 g, 4.65 mmol, 1 equiv) and paraformaldehyde (1.39 g, 46.5 mmol, 10 equiv) were taken together in a mixture of 1,2-Dichloroethane (20 mL, 10 vol) and DMF (10 mL, 5 vol) under nitrogen atmosphere. To this, acetic acid (0.8 mL, 13.95 mmol, 3 equiv) was added and the reaction mixture was stirred for 1 hour at room temperature. NaCNBH₃ (1.15 g, 18.6 mmol, 4 equiv) was added then in portions and the reaction mixture was stirred for additional 16 hours at room temperature. Upon completion of the reaction (as confirmed by TLC analysis, 100% EtOAc R_{f}˜0.4, and LCMS), the reaction mixture was diluted with DCM (100 mL) and washed water (3×50 mL) and brine (100 mL), dried over Na₂SO₄, filtered and concentrated. The resulted residue was purified by chromatography (column size: Biotage R snap cartridge, KP-Sil, 50 g, 230-400 silica gel) using 40-45% ethyl acetate in dichloromethane to obtain tert-Butyl(4-((2-(2,6-dioxopiperidin-3-yl)-1-oxoisoindolin-4-yl)(methyl)amino)butyl)carbamate (800 mg, 39%) as white solid.

¹H-NMR (400 MHz, DMSO-d₆): δ 11.02 (s, 1H), 7.37 (t, J=7.60 Hz, 1H), 7.15 (d, J=7.20 Hz, 1H), 6.99 (d, J=8.00 Hz, 1H), 6.80 (t, J=5.60 Hz, 1H), 5.08-5.13 (m, 1H), 4.33-4.51 (m, 2H), 2.90-2.96 (m, 3H), 2.88 (s, 3H), 2.52-2.62 (m, 2H), 2.01-2.03 (m, 1H), 1.42-1.48 (m, 4H), 1.40 (s, 9H). LC-MS: 445.4 (M-H), Rt (min): 2.525, Area %—99.11.

### Step 2: 3-(4-((4-Aminobutyl)(methyl)amino)-1-oxoisoindolin-2-yl)piperidine-2,6-dione hydrochloride

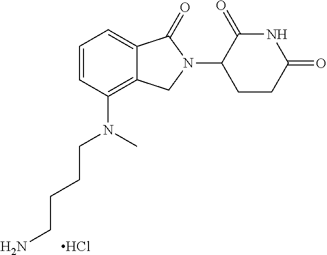 [SMILES: CN(CCCCN)c1cccc2c1CN(C1CCC(=O)NC1=O)C2=O ; Cl]

To an ice cold solution of tert-Butyl(4-((2-(2,6-dioxopiperidin-3-yl)-1-oxoisoindolin-4-yl)(methyl)amino)butyl)carbamate 5 (800 mg, 1.80 mmol) in DCM (10 mL), was added HCl in EtOAc (1 M, 8 mL) and stirred for 4 hours at room temperature. The progress of the reaction was monitored by LCMS. The reaction mixture was concentrated and lyophilized to afford 3-(4-((4-Aminobutyl)(methyl)amino)-1-oxoisoindolin-2-yl)piperidine-2,6-dione hydrochloride (550 mg, 88%) pale yellow solid.

¹H-NMR (400 MHz, DMSO-d₆): δ 11.01 (s, 1H), 7.99 (brs, 3H), 7.37-7.51 (m, 3H), 5.10-5.15 (m, 1H), 4.44-4.63 (m, 2H), 3.30 (s, 2H), 3.01 (s, 3H), 2.92-2.98 (m, 2H), 2.60-2.78 (m, 3H), 2.45-2.50 (m, 1H), 2.06-2.08 (m, 1H), 1.66-1.67 (m, 4H). ¹³C-NMR (100 MHz, DMSO-d₆): δ 173.34, 171.30, 167.67, 134.36, 133.03, 130.23, 117.06, 122.84, 55.43, 52.21, 48.02, 42.50, 31.65, 23.02, 22.96. LCMS: 345.1 (M+H). Method: Mobile Phase A: 0.1% Formic Acid in H₂O. Mobile Phase B: Acetonitrile. Flow rate: 1.5 mL/min. COLUMN: Atlantis dC18 (50×4.6) mm, 5μ. Rt (min): 1.252; Area %—98.76. HPLC Method: Mobile Phase A: 0.1% TFA in H₂O. Mobile Phase B: Acetonitrile. Flow rate: 1.0 mL/min. COLUMN: Atlantis dC18 (250×4.6) mm, 5 μm. Rt (min): 6.313; Area %—97.81.

### Hydrochloride Salt Form of Compound 3

### 3-(4-((4-Aminobutyl)(isopropyl)amino)-1-oxoisoindolin-2-yl)piperidine-2,6-dione hydrochloride

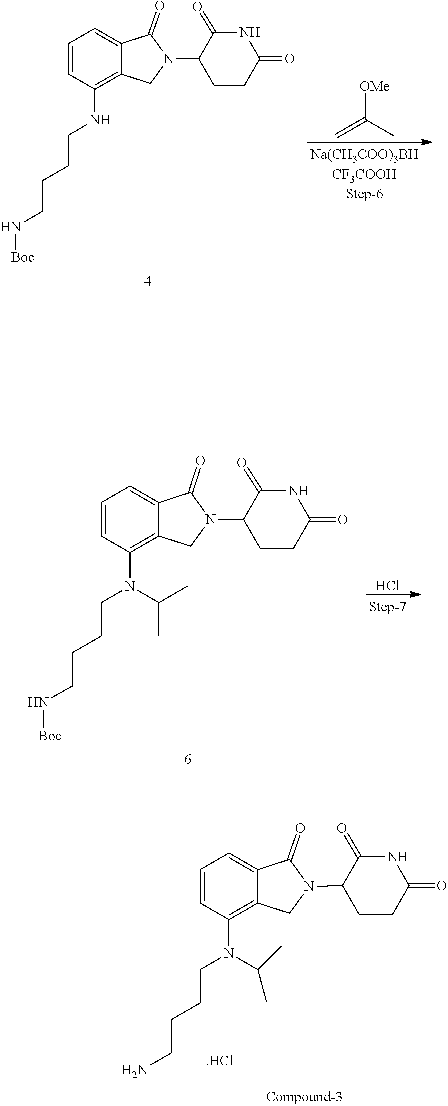

### Step-1: tert-Butyl(4-((2-(2,6-dioxopiperidin-3-yl)-1-oxoisoindolin-4-yl)(isopropyl)amino)butyl)carbamate

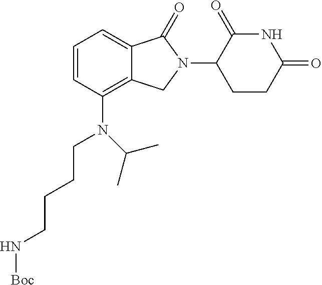 [SMILES: CC(C)N(CCCCNC(=O)OC(C)(C)C)c1cccc2c1CN(C1CCC(=O)NC1=O)C2=O]

tert-Butyl(4-((2-(2,6-dioxopiperidin-3-yl)-1-oxoisoindolin-4-yl)amino)butyl)carbamate 4 (1.5 g, 3.4 mmol, 1 equiv) and 2-methoxypropene (1.25 g, 17.5 mmol, 5 equiv) were taken together in 1,2-Dichloroethane (30 mL, 20 vol) and the suspension was cooled to 0° C. To this was added triflouroacetic acid (0.22 mL, 3.0 mmol, 0.9 equiv) in drops. Then sodium triacetoxyborohydride (1.07 g, 5.1 mmol, 1.5 equiv) was added in portions at 0° C. under nitrogen atmosphere and the resulted reaction mixture was stirred for 16 hours at room temperature. Upon completion of the reaction (as confirmed by TLC analysis, 100% EtOAc R_{f}˜0.5, and LCMS), the reaction mixture was diluted with DCM (100 mL) and washed with water (2×100 mL) and brine (100 mL), dried over Na₂SO₄, filtered and concentrated under vacuum. The crude product was purified by chromatography (column size: Biotage R snap cartridge, KP-Sil, 50 g, 230-400 silica gel) using 40-45% Ethylacetate in dichloromethane to afford tert-Butyl(4-((2-(2,6-dioxopiperidin-3-yl)-1-oxoisoindolin-4-yl)(isopropyl)amino)butyl)carbamate (620 mg, 38%) as white solid.

LCMS: 473.4 (M+H). Method: Column: Atlantis dC18 (50×4.6) 5μ, Mobile phase: A: 0.1% Formic Acid in H₂O B: ACN, Flow Rate: 1.5 mL/min. Rt (min): 2.454; Area %—97.90.

### Step 2: 3-(4-((4-Aminobutyl(isopropyl)amino)-1-oxoisoindolin-2-yl)piperidine-2,6-dione hydrochloride

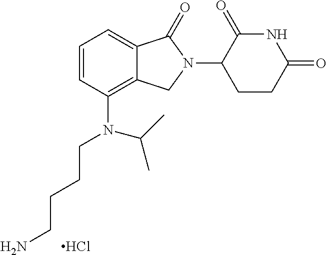 [SMILES: CC(C)N(CCCCN)c1cccc2c1CN(C1CCC(=O)NC1=O)C2=O ; Cl]

To an ice cold solution of tert-Butyl (4-((2-(2,6-dioxopiperidin-3-yl)-1-oxoisoindolin-4-yl)(isopropyl)amino)butyl)carbamate 6 (620 mg, 1.313 mmol) in DCM (4 mL) was added HCl in EtOAc (1 M, 4 mL) and stirred for 12 hours at room temperature. The progress of the reaction was monitored by LCMS. The reaction mixture was concentrated under vacuum and further lyophilized to give the desired compound 3-(4-((4-Aminobutyl(isopropyl)amino)-1-oxoisoindolin-2-yl)piperidine-2,6-dione hydrochloride (440 mg, 96%) as pale brown solid.

¹H-NMR (400 MHz, DMSO-d₆): δ 11.05 (s, 1H), 7.98 (s, 3H), 7.72 (s, 2H), 5.12-5.08 (m, 1H), 4.56 (dd, J=18.00, 56.40 Hz, 2H), 3.82-3.51 (m, 1H), 3.47 (s, 2H), 2.90-2.70 (m, 1H), 2.68-2.65 (m, 3H), 2.44-2.41 (m, 1H), 2.09 (d, J=5.20 Hz, 1H), 1.53 (t, J=7.60 Hz, 2H), 1.33 (d, J=6.80 Hz, 2H), 1.23 (s, 6H). ¹³C-NMR (100 MHz, DMSO-d₆): 173.37, 171.25, 167.38, 136.43, 134.63, 130.36, 126.85, 123.25, 60.12, 52.33, 49.80, 48.17, 40.49, 39.24, 38.53, 31.55, 24.53, 22.91, 18.92, 18.62. LCMS: 373.2 (M-HCl). Method: Column: Atlantis dC18 (50×4.6) 5μ, Mobile phase: A: 0.1% Formic Acid in H₂O B: ACN, Flow Rate: 1.5 mL/min. Rt (min): 1.239; Area %—97.330. HPLC: 97.905%, Rt (min): 6.141.

### Hydrochloride Salt Form of Compound 4

### 3-(4-((4-Aminobutyl)(propyl)amino)-1-oxoisoindolin-2-yl)piperidine-2,6-dione hydrochloride

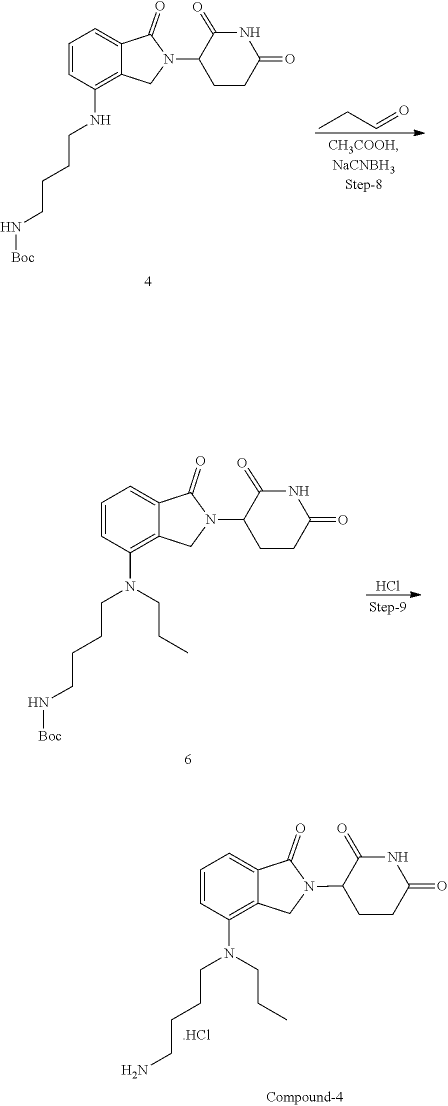

### Step-1: tert-Butyl(4-((2-(2,6-dioxopiperidin-3-yl)-1-oxoisoindolin-4-yl)(propyl)amino)butyl)carbamate

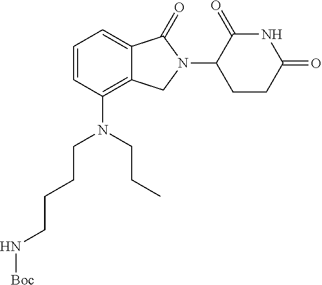 [SMILES: CCCN(CCCCNC(=O)OC(C)(C)C)c1cccc2c1CN(C1CCC(=O)NC1=O)C2=O]

tert-Butyl(4-((2-(2,6-dioxopiperidin-3-yl)-1-oxoisoindolin-4-yl)amino)butyl)carbamate 4 (1.5 g, 3.4 mmol, 1 equiv) and propionaldehyde (2.0 g, 3.4 mmol, 10 equiv) were dissolved in a mixture of 1,2-Dichloroethane (20 mL) and DMF (10 mL) under nitrogen atmosphere. To this was added acetic acid (0.58 mL, 10.2 mmol, 3 equiv) and the resulted reaction mixture was stirred for 1 hour at room temperature. At this point, sodium cyanoborohydride (0.84 g, 1.3 mmol, 4 equiv) was added in portions and stirred for 16 hours at room temperature. Upon completion of the reaction (as confirmed by TLC analysis, 100% EtOAc R_{f}˜0.4, and LCMS), the reaction mixture was diluted with DCM (100 mL) and washed water (3×100 mL) and brine (100 mL), dried over Na₂SO₄, filtered and concentrated. The resulted residue was purified by chromatography (Isolera, column size: Biotage R snap cartridge, KP-Sil, 50 g, 230-400 silica gel) using 40-45% EtOAc in DCM to obtain tert-Butyl(4-((2-(2,6-dioxopiperidin-3-yl)-1-oxoisoindolin-4-yl)(propyl)amino)butyl)carbamate 7 (600 mg, 37%) as white solid.

LCMS: 473.4 (M+H). Method: Column: Atlantis dC18 (50×4.6) 5μ, Mobile phase: A: 0.1% Formic Acid in H₂O B: ACN, Flow Rate: 1.5 mL/min. Rt (min): 2.709; Area %—99.720.

### Step 2: 3-(4-((4-Aminobutyl)(propyl)amino)-1-oxoisoindolin-2-yl)piperidine-2,6-dione hydrochloride

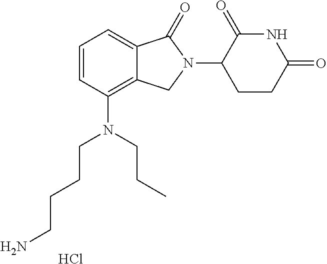 [SMILES: CCCN(CCCCN)c1cccc2c1CN(C1CCC(=O)NC1=O)C2=O ; Cl]

To an ice cold solution of tert-Butyl (4-((2-(2,6-dioxopiperidin-3-yl)-1-oxoisoindolin-4-yl)(propyl)aminuteso)butyl)carbamate 7 (600 mg, 1.2695 mmol, 1 equiv) in DCM (10 mL) was added HCl in EtOAc (1M, 5 mL) and stirred for 4 hours at room temperature. The progress of the reaction was monitored by LCMS. The reaction mixture was concentrated and further lyophilized to afford 3-(4-((4-Aminobutyl)(propyl)amino)-1-oxoisoindolin-2-yl)piperidine-2,6-dione hydrochloride (360 mg, 68%) as pale yellow solid.

¹H-NMR (400 MHz, DMSO-d₆): δ 11.01 (s, 1H), 7.99 (s, 3H), 7.53 (s, 2H), 5.12 (q, J=4.80 Hz, 1H), 4.49 (d, J=36.40 Hz, 2H), 3.29 (s, 1H), 2.97 (s, 1H), 2.96-2.88 (m, 1H), 2.72 (q, J=22.80 Hz, 2H), 2.62 (d, J=16.40 Hz, 1H), 2.44 (s, 1H), 2.06 (t, J=5.20 Hz, 1H), 2.05 (s, 1H), 1.55 (s, 6H), 0.84 (t, J=7.20 Hz, 3H). ¹³C-NMR (100 MHz, DMSO-d₆): δ 173.34, 171.27, 167.49, 162.82, 134.55, 130.45, 125.14, 57.35, 55.02, 52.34, 48.12, 38.58, 36.28, 31.59, 31.26, 24.51, 22.94, 21.57, 19.04, 11.25. LCMS: 373.1 (M-HCl). Method: Column: Atlantis dC18 (50×4.6) 5μ, Mobile phase: A: 0.1% Formic Acid in H₂O B: ACN, Flow Rate: 1.5 mL/min. Rt (min): 1.386; Area %—98.551. HPLC: 98.878%, Rt (min): 6.95.

### Hydrochloride Salt Form of Compound 6

### 3-(4-((2-(1-(Aminomethyl)cyclohexyl)ethyl)amino)-1-oxoisoindolin-2-yl)piperidine-2,6-dione hydrochloride

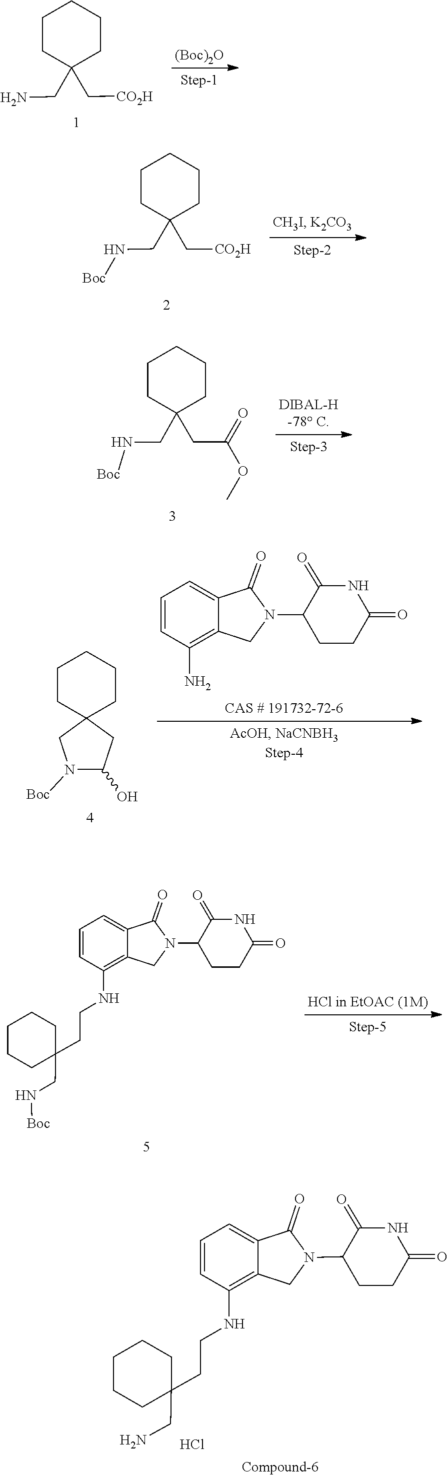

### Step-1: 2-(1-(((tert-Butoxycarbonyl)amino)methyl)cyclohexyl)acetic acid

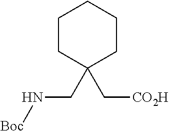 [SMILES: CC(C)(C)OC(=O)NCC1(CC(=O)O)CCCCC1]

To a solution of 2-(1-(Aminomethyl)cyclohexyl)acetic acid 1 (30 g, 175.4 mmol, 1 equiv) in THF (300 mL, 10 vol) were added a solution of NaOH (7.0 g, 175.4 mmol, 1 equiv) in water (100 mL, 5 vol) and boc-anhydride (38.2 g, 175.4 mmol, 1 equiv) in drops at 0° C. The mixture was stirred at room temperature for 16 hours. Upon completion of the reaction (as confirmed by TLC analysis, 20% EtOAc in pet ether, R_{f}˜0.4, and LCMS-ELSD), the solvent was evaporated to dryness and diluted with water (100 mL) and washed with ethyl acetate (1×150 mL). The aqueous layer was separated and acidified with 1.5N HCl to pH 5 to 6 and extracted with DCM (2×500 mL). The organic layer was washed with brine (250 mL), dried over Na₂SO₄, filtered and concentrated under vacuum to afford 2-(1-(((tert-Butoxycarbonyl)amino)methyl)cyclohexyl)acetic acid 2 (35 g, 75%) as white solid. LCMS (ELSD): 270.3 (M-H). Method: Column: Atlantis dC18 (50×4.6) 5μ, Mobile phase: A: 0.1% Formic Acid in H₂O B: ACN, Flow Rate: 1.5 mL/min. Rt (min): 2.950; Area %—99.502.

### Step 2: Methyl 2-(1-(((tert-butoxycarbonyl)amino)methyl)cyclohexyl)acetate

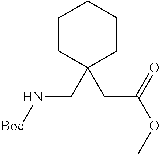 [SMILES: COC(=O)CC1(CNC(=O)OC(C)(C)C)CCCCC1]

To a solution of 2-(1-(((tert-Butoxycarbonyl)amino)methyl)cyclohexyl)acetic acid 2 (20 g, 73.8 mmol, 1 equiv) in DMF (200 mL, 10 vol) were added potassium carbonate (20.3 g, 147.6 mmol, 2 equiv) and methyl Iodide (26 mL, 369 mmol, 5 equiv) in drops at 0° C. The mixture was stirred at room temperature for 16 hours. Upon completion of the reaction (as confirmed by TLC analysis, 20% EtOAc in pet ether, R_{f}˜0.6, and LCMS-ELSD), the reaction mixture was diluted with ethyl acetate (200 mL) and washed with ice cold H₂O (3×500 mL). The organic layer was washed with brine (1×500 mL), dried over Na₂SO₄, filtered and concentrated under vacuum to afford Methyl 2-(1-(((tert-butoxycarbonyl)amino)methyl)cyclohexyl)acetate 3 (15 g, 71%) as a pale yellow liquid.

LCMS (ELSD): 230.3 (M-tert-Butyl). Method: Column: Atlantis dC18 (50×4.6) 5μ, Mobile phase: A: 0.1% Formic Acid in H₂O B: ACN, Flow Rate: 1.5 mL/min. Rt (min): 3.374; Area %—99.76.

### Step-3: tert-Butyl 3-hydroxy-2-azaspiro[4.5]decane-2-carboxylate

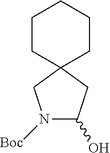 [SMILES: CC(C)(C)OC(=O)N1CC2(CCCCC2)CC1O]

To a solution of Methyl 2-(1-(((tert-butoxycarbonyl)amino)methyl)cyclohexyl)acetate 3 (8 g, 28.07 mmol, 1 equiv) in DCM (150 mL) was added DIBAL-H (1 M in hexane, 28.07 mL, 28.07 mmol, 1 equiv) in drops at −78° C. The mixture was stirred at 0° C. for 2 hours.

Upon completion of the reaction (as confirmed by TLC analysis, 20% EtOAc in pet ether, R_{f} 0.5, and LCMS-ELSD), the reaction mixture was quenched with saturated NH₄Cl solution (50 mL) and was filtered through a celite bed and washed with DCM (250 mL). The separated organic layer was washed with brine (250 mL), dried over Na₂SO₄, filtered and concentrated under vacuum below 35° C. to afford tert-Butyl 3-hydroxy-2-azaspiro[4.5]decane-2-carboxylate 4 (6 g, crude) as pale brown liquid which was used without purification.

LCMS (ELSD): 202.1 (M-tert-Butyl). Method: Column: Atlantis dC18 (50×4.6) 5μ, Mobile phase: A: 0.1% Formic Acid in H₂O B: ACN, Flow Rate: 1.5 mL/min. Rt (min): 2.187; Area %—99.976.

### Step-4: tert-Butyl((1-(2-((2-(2,6-dioxopiperidin-3-yl)-1-oxoisoindolin-4-yl)amino)ethyl)cyclohexyl)methyl)carbamate

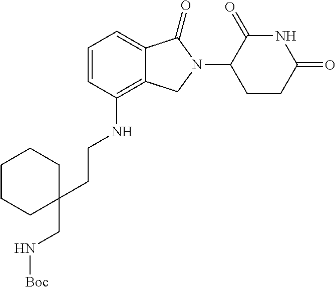 [SMILES: CC(C)(C)OC(=O)NCC1(CCNc2cccc3c2CN(C2CCC(=O)NC2=O)C3=O)CCCCC1]

To a stirred solution of Lenalidomide (CAS #191732-72-6, 2 g, 7.6 mmol, 1 equiv) and tert-Butyl 3-hydroxy-2-azaspiro[4.5]decane-2-carboxylate 4 (5.79 g, 2.3 mmol) in mixture of 1,2-Dichloroethane (20 mL) and DMF (20 mL) was added acetic acid (1.82 mL, 30.7 mmol, 4 equiv) under nitrogen atmosphere. The resulted reaction mixture was stirred for 1 hour at room temperature. Then the reaction mixture was cooled to 0° C. and sodium cyanoborohydride (1.84 g, 30.7 mmol, 4 equiv) was added portion-wise and stirred for 16 hours at room temperature. Upon completion of the reaction (as confirmed by TLC analysis, 100% EtOAc, R_{f}˜0.6, and LCMS), the reaction mixture was diluted with DCM (100 mL) and washed water (3×50 mL) and brine (100 mL), dried over Na₂SO₄, filtered and concentrated. The resulted residue was purified by chromatography (column size: Biotage R snap cartridge, KP-Sil, 25 g, 230-400 silica gel) using 40-45% EtOAc in DCM to afford tert-Butyl((1-(2-((2-(2,6-dioxopiperidin-3-yl)-1-oxoisoindolin-4-yl)amino)ethyl)cyclohexyl)methyl)carbamate 5 (620 mg, 16%) as white solid.

LCMS: 443.4 (M-t-Bu). Method: Column: Atlantis dC18 (50×4.6) 5μ,

Mobile phase: A: 0.1% Formic Acid in H₂O B: ACN, Flow Rate: 1.5 mL/min. Rt (min): 3.072; Area % —98.042.

### Step-5:3-(4-((2-(1-(Aminomethyl)cyclohexyl)ethyl)amino)-1-oxoisoindolin-2-yl)piperidine-2,6-dione hydrochloride

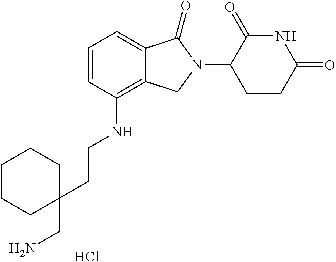 [SMILES: NCC1(CCNc2cccc3c2CN(C2CCC(=O)NC2=O)C3=O)CCCCC1 ; Cl]

To an ice cold solution of tert-Butyl((1-(2-((2-(2,6-dioxopiperidin-3-yl)-1-oxoisoindolin-4-yl)amino)ethyl)cyclohexyl)methyl)carbamate 5 (620 mg, 1.2437 mmol, 1 equiv) in DCM (10 mL) was added HCl in EtOAc (1 M, 6 mL) and stirred for 12 hours at room temperature. After confirming the completion of reaction by LCMS, the reaction mixture was concentrated under vacuum to give the crude which was co-distilled with Milli-Q H₂O (2×10 mL) and dried under vacuum to afford 3-(4-((2-(1-(Aminomethyl)cyclohexyl)ethyl)amino)-1-oxoisoindolin-2-yl)piperidine-2,6-dione hydrochloride (480 mg, 98%) as pale yellow solid.

¹H-NMR (400 MHz, DMSO-d₆): δ 11.03 (s, 1H), 8.05 (s, 3H), 7.43-7.31 (m, 1H), 7.10 (d, J=7.20 Hz, 1H), 7.01 (d, J=7.60 Hz, 1H), 5.12 (q, J=5.20 Hz, 1H), 4.39 (d, J=17.20 Hz, 1H), 4.24 (d, J=17.60 Hz, 1H), 3.18-2.97 (m, 2H), 2.94-2.89 (m, 1H), 2.79 (d, J=5.60 Hz, 2H), 2.63 (d, J=17.20 Hz, 1H), 2.31 (q, J=4.40 Hz, 1H), 2.06-2.04 (m, 1H), 1.77 (t, J=7.20 Hz, 2H), 1.44 (s, 10H). ¹³C-NMR (100 MHz, DMSO-d₆): δ 173.32, 171.43, 168.37, 138.77, 133.32, 131.39, 129.94, 119.19, 116.76, 52.15, 46.70, 45.18, 41.16, 40.56, 34.88, 33.03, 31.64, 31.49, 25.84, 23.21, 21.11. LCMS: 399.1 (M-HCl). Method: Column: Atlantis dC18 (50×4.6) 5μ, Mobile phase: A: 0.1% Formic Acid in H₂O B: ACN, Flow Rate: 1.5 mL/min. Rt (min): 1.551; Area %—99.47. HPLC: 99.499%, Rt (min): 8.979.

### Hydrochloride Salt Form of Compound 46

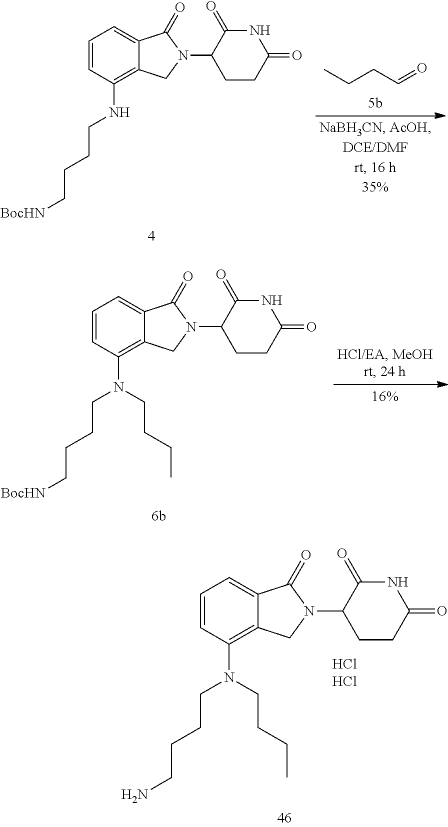

### General Procedure for the Preparation of Compound 6b

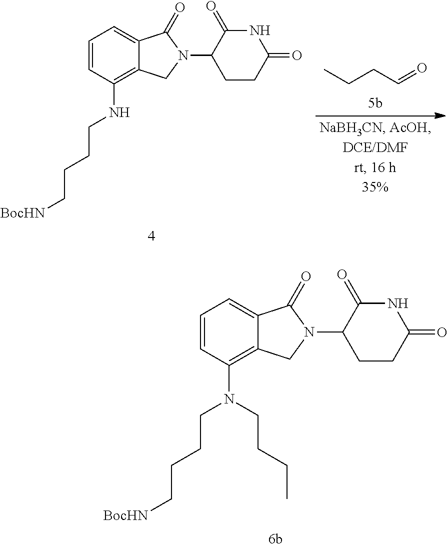

To a solution of compound 4 (1.0 g, 2.32 mmol, 1 eq.) and compound 5b (336 mg, 4.65 mmol, 2.0 eq.) in DCE/DMF (10 ml) was added acetic acid (1.0 ml), molecular sieve (1.0 g) and stirred for 1 h at room temperature under nitrogen atmosphere. NaBH₃CN (585 mg, 9.3 mmol, 4 eq.) was added in portions and stirred for 16 h at room temperature under nitrogen atmosphere. Upon completion of the reaction (as confirmed by TLC analysis, 100% EA and LCMS), the reaction was diluted with DCM (20 mL). The mixture was washed with water (3×20 mL) and brine (20 mL), dried over Na₂SO₄. The resulting mixture was concentrated and purified by silica gel column chromatography (DCM/EA: 2/1) and P-HPLC (acetonitrile/H₂O: 40%-60%) to afford compound 6b (400 mg, 35%) as a white solid.

TLC: EA=100%

R_{f} (Compound 4)=0.5

R_{f} (Compound 6b)=0.6

LC-MS: 487.3 [M+1]⁺

### General Procedure for the Preparation of Compound 46

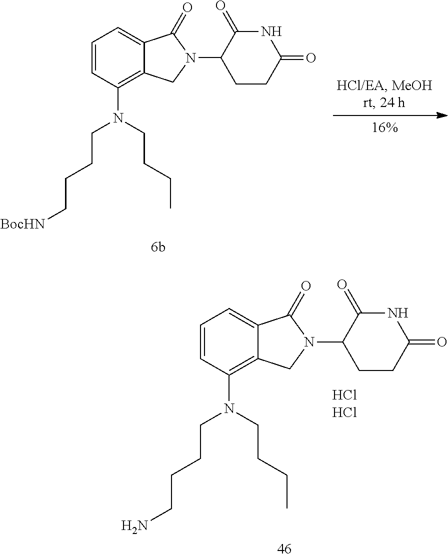 [SMILES: CCCCN(CCCCNC(=O)OC(C)(C)C)c1cccc2c1CN(C1CCC(=O)NC1=O)C2=O ; CCCCN(CCCCN)c1cccc2c1CN(C1CCC(=O)NC1=O)C2=O]

To a solution of compound 6b (400 mg, 0.873 mmol, 1 eq.) in methanol (1 ml) was added HCl/EA (2N, 8.73 mmol, 10 eq.). The resulting solution was stirred at room temperature for 24 h. The resulting mixture was concentrated under reduced pressure. The residue was purified by P-HPLC (acetonitrile/H₂O: 30%-40%) to afford compound 46 (60 mg, 16%) as a white solid.

TLC: DCM/methanol=10/1

R_{f} (Compound 6b)=0.6

R_{f} (Compound 46)=0.2

LC-MS: 387.25 [M+1]⁺

¹H NMR (400 MHz, D₂O) δ 7.84 (s, 1H), 7.70 (s, 2H), 5.07 (d, J=8.7 Hz, 1H), 4.55 (s, 3H), 3.51 (s, 4H), 3.18 (s, 1H), 2.73 (d, J=7.9 Hz, 4H), 2.41 (d, J=7.5 Hz, 1H), 2.16 (s, 1H), 1.31 (dd, J=93.7, 45.1 Hz, 8H), 0.65 (s, 3H).

### Hydrochloride Salt Form of Compound 47

### 3-(4-((4-aminobutyl)(pentyl)amino)-1-oxoisoindolin-2-yl)piperidine-2,6-dione hydrochloride

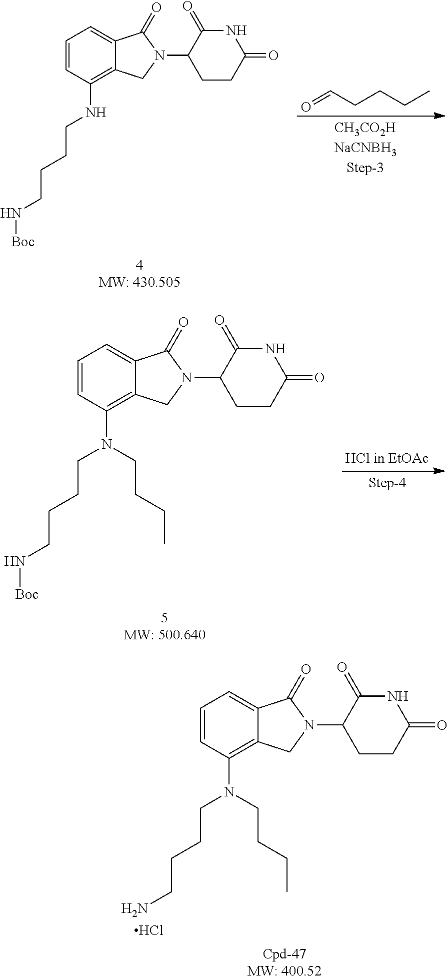

### Step-3: tert-Butyl(4-((2-(2,6-dioxopiperidin-3-yl)-1-oxoisoindolin-4-yl)(methyl)amino)butyl)carbamate

To a solution of tert-Butyl(4-((2-(2,6-dioxopiperidin-3-yl)-1-oxoisoindolin-4-yl)amino)butyl) carbamate 4 (3 g, 6.97 mmol, 1 equiv) and pentanal (6.0 g, 69.7 mmol, 10 equiv) in a mixture of 1,2-dichloroethane (60 mL, 20 vol) and DMF (30 mL, 10 vol) was added acetic acid (1.2 mL, 20.91 mmol, 3 equiv) and the reaction mixture was stirred for 2 hour at room temperature. NaCNBH₃ (1.75 g, 27.9 mmol, 4 equiv) was added in portions and the reaction mixture was stirred for additional 16 h at room temperature. TLC analysis (70% EtOAc in pet-ether) indicated product formation and the starting material was not consumed completely. Additional quantities of pentanal (2×6.0 g, 69.7 mmol, 10 equiv), acetic acid (2×0.8 mL, 13.94 mmol, 2 equiv) and NaCNBH₃ (0.87 g, 13.94 mmol, 2 equiv) were added and the resulted reaction mixture was stirred for 36 hours at room temperature. Upon completion of the reaction (as confirmed by TLC analysis, 70% EtOAc in pet ether, R_{f}˜0.6, and LCMS), the reaction mixture was diluted with DCM (100 mL), washed with water (3×100 mL), brine (300 mL), dried over anhy. Na₂SO₄, filtered and concentrated. The resulted residue was purified by chromatography (Biotage R snap cartridge, KP-Sil, 100 g, 230-400 silica gel) with 65-70% ethyl acetate in Pet-ether to get tert-butyl (4-((2-(2,6-dioxopiperidin-3-yl)-1-oxoisoindolin-4-yl)(pentyl)amino)butyl)carbamate 5 (2.5 g, 97%) as white solid.

¹H-NMR (400 MHz, DMSO-d₆): δ (s, 1H), 7.37 (t, J=8.00 Hz, 1H), 7.18 (d, J=7.20 Hz, 1H), 7.07 (d, J=8.00 Hz, 1H), 6.78 (t, J=5.60 Hz, 1H), 5.11 (q, J=5.20 Hz, 1H), 4.42-4.27 (m, 2H), 3.17 (t, J=8.40 Hz, 4H), 2.96-2.90 (m, 3H), 2.68-2.61 (m, 2H), 2.03-2.00 (m, 1H), 1.43 (s, 15H), 1.38 (t, J=10.80 Hz, 4H), 0.86 (s, 3H).

LC-MS: 501.3 (M+H), Rt (min): 1.853, Area %—97.842.

### Step 4: 3-(4-((4-aminobutyl)(pentyl)amino)-1-oxoisoindolin-2-yl)piperidine-2,6-dione hydrochloride

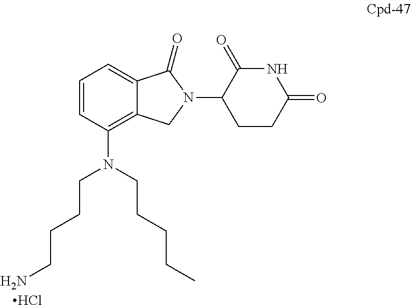 [SMILES: CCCCCN(CCCCN)c1cccc2c1CN(C1CCC(=O)NC1=O)C2=O ; Cl]

To an ice cold solution of tert-butyl (4-((2-(2,6-dioxopiperidin-3-yl)-1-oxoisoindolin-4-yl)(pentyl)amino)butyl)carbamate (2.5 g, 4.99 mmol) in DCM (25 mL), was added HCl (4M soln. in EtOAc, 12.5 mL) and stirred for 2 h at room temperature. Upon completion of the reaction (as confirmed by TLC analysis, 100% EtOAc R_{f}˜0.4), the reaction mixture was concentrated and lyophilized to afford 3-(4-((4-aminobutyl)(pentyl)amino)-1-oxoisoindolin-2-yl)piperidine-2,6-dione Hydrochloride 47 (2.4 g, 98%) as off-white solid.

¹H-NMR (400 MHz, DMSO-d₆): δ 11.01 (s, 1H), 7.92 (s, 4H), 7.39 (s, 2H), 5.15-5.10 (m, 1H), 3.28 (s, 4H), 2.98-2.92 (m, 1H), 2.75 (d, J=5.20 Hz, 2H), 2.68-2.64 (m, 2H), 2.08-2.04 (m, 1H), 1.54 (s, 6H), 1.26 (d, J=13.60 Hz, 5H), 0.87-0.81 (m, 3H).

LCMS: 401.2 (M+H). Method: Mobile Phase A: 0.1% TFA in H₂O. Mobile Phase B: 0.1% TFA in ACN. Flow rate: 1.5 mL/min. Column: XBridge C8 (50×4.6 mm) 3.5 μm. Rt (min): 1.356; Area %: 96.152.

HPLC: Method: Mobile Phase A: 0.1% TFA in H₂O. Mobile Phase B: Acetonitrile. Flow rate: 2.0 mL/min. Column: X-Bridge C8 (50×4.6) mm, 3.5 μm. Rt (min): 2.341; Area %: 99.332.

### Hydrochloride Salt Form of Compound 48

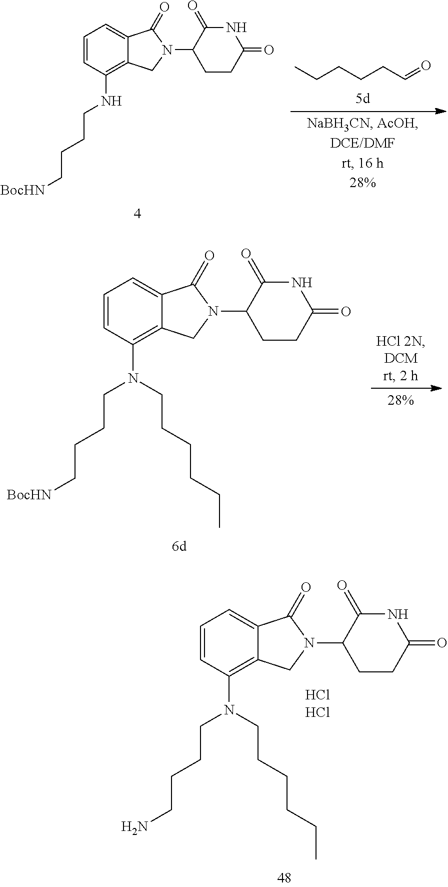

### General Procedure for the Preparation of Compound 6d

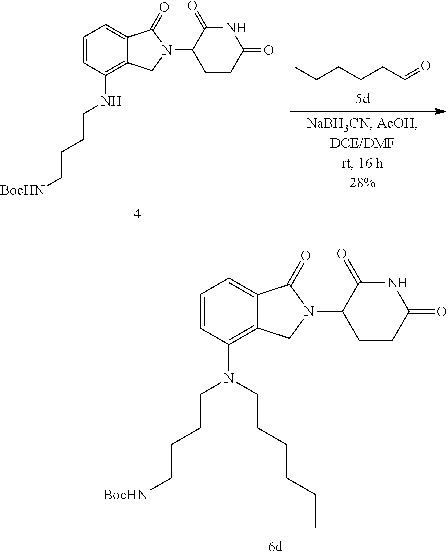

To a solution of compound 4 (2.0 g, 4.65 mmol, 1 eq.) and compound 5d (932 mg, 9.30 mmol, 2.0 eq.) in DCE/DMF (20 ml) was added acetic acid (2.0 ml), molecular sieve (1.0 g) and stirred for 1 h at room temperature under nitrogen atmosphere. NaBH₃CN (1.17 g, 18.6 mmol, 4 eq.) was added in portions and stirred for 16 h at room temperature under nitrogen atmosphere. Upon completion of the reaction (as confirmed by TLC analysis, 100% EA and LCMS), the reaction was diluted with DCM (20 mL). The mixture was washed with water (3×30 mL) and brine (30 mL), dried over Na₂SO₄. The resulting mixture was concentrated under reduced pressure and purified by silica gel column chromatography (DCM/EA: 2/1) and P-HPLC (acetonitrile/H₂O: 40%-60%) to afford compound 6 (660 mg, 28%) as a white solid.

TLC: EA=100%

R_{f} (Compound 4)=0.5

R_{f} (Compound 6d)=0.6

LC-MS: 515.40 [M+1]⁺

### General Procedure for the Preparation of Compound 48

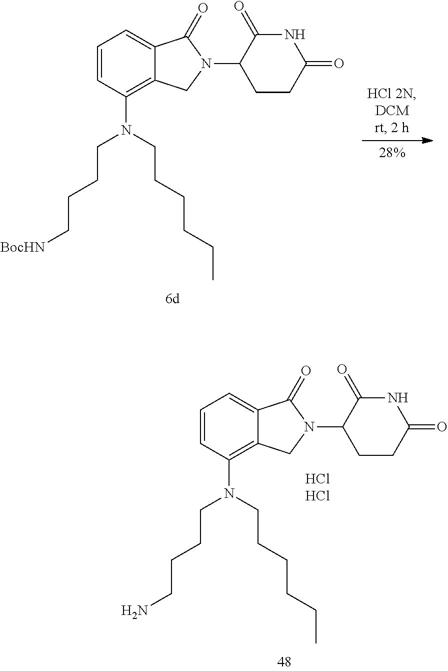 [SMILES: CCCCCCN(CCCCNC(=O)OC(C)(C)C)c1cccc2c1CN(C1CCC(=O)NC1=O)C2=O ; CCCCCCN(CCCCN)c1cccc2c1CN(C1CCC(=O)NC1=O)C2=O]

To a solution of compound 6d (660 mg, 1.28 mmol, 1 eq.) in DCM (6 mL) was added HCl/EA (2N, 6.42 mmol, 5 eq.). The resulting solution was stirred at 25° C. for 2 h. The resulting mixture was concentrated under reduced pressure. The residue was purified by P-HPLC (acetonitrile/H₂O: 30%-40%) to afford compound 48 (150.55 mg, 28%) as a white solid.

TLC: DCM/methanol=10/1

R_{f} (Compound 6d)=0.6

R_{f} (Compound 48)=0.2

LC-MS: 415.40 [M+1]⁺

¹H NMR (400 MHz, CDCl₃): δ 10.25 (s, 1H), 7.75 (s, 3H), 7.40-7.28 (m, 2H), 6.98 (s, 1H), 5.17 (d, J=8.9 Hz, 1H), 4.40 (dd, J=58.4, 16.5 Hz, 2H), 3.13-2.54 (m, 9H), 2.03 (d, J=46.6 Hz, 7H), 1.63 (s, 2H), 1.31 (d, J=63.7 Hz, 13H), 0.85 (s, 3H).

### Hydrochloride Salt Form of Compound 49

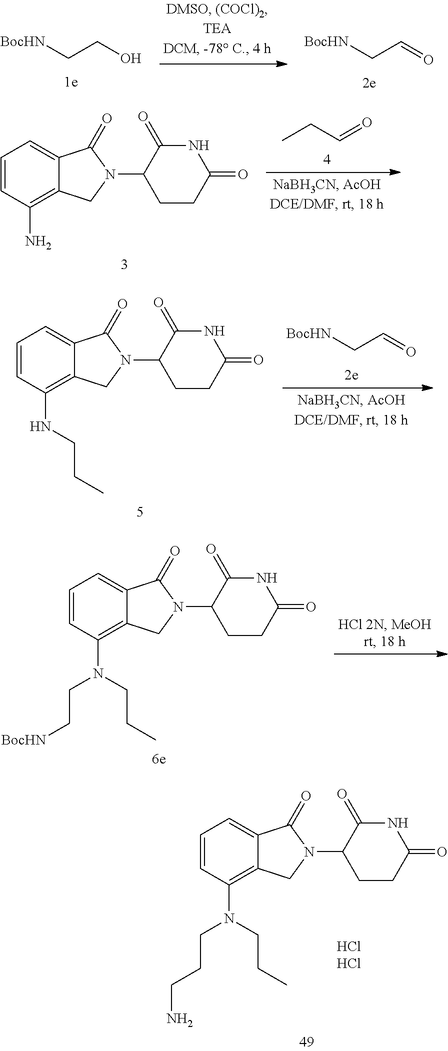

### General Procedure for the Preparation of Compound 2e

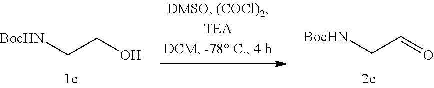 [SMILES: CC(C)(C)OC(=O)NCCO ; CC(C)(C)OC(=O)NCC=O]

To a solution of Oxalyl chloride (9.5 g, 74.5 mmol, 1.2 eq.) in DCM (50 mL) was added dropwise to a solution of DMSO (10 mL) in DCM (100 mL) at −78° C. over a period of 15 minutes under nitrogen atmosphere and stirred for 15 minutes at the same temperature. The solution of Compound 1e (10.0 g, 62.1 mmol, 1.0 eq.) in DCM (50 mL) was added dropwise over a period of 15 minutes. After stirring the reaction mixture for 30 minutes at −78° C., was added TEA (18.8 g, 188.2 mmol, 3.0 eq.) in drops at −78° C. Once addition was complete, the reaction mixture was slowly warmed to 0° C. and stirred for 30 minutes. Upon completion of the reaction (as confirmed by TLC analysis, PE/EA=4/1, R_{f}˜0.4, KMnO₄ or Ninhydrin), the reaction mixture was diluted with DCM (50 mL). The mixture was washed with 10% citric acid (500 mL), water (50 mL) and brine (50 mL), dried over Na₂SO₄, filtered and concentrated under vacuum below 30° C. to afford Compound 2 (11.4 g, crude) as yellow oil which was used without further purification.

TLC: PE/EA=4:1, KMnO₄, Ninhydrin

R_{f} (compound 1e)=0.2

R_{f} (compound 2e)=0.4

LC-MS: No found.

### General Procedure for the Preparation of Compound 5

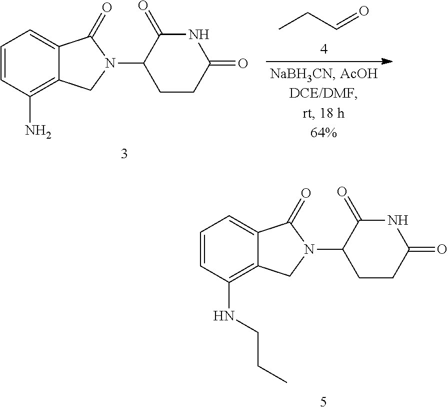

To a solution of compound 3 (0.2 g, 3.9 mmol, 1.0 eq.) and compound 4 (1.0 g, 3.9 mmol, 1.0 eq.) in DCE/DMF (1/1, 20 ml) was added acetic acid (0.7 g, 11.6 mmol, 3.0 eq.). The reaction was stirred for 2 h at room temperature. At this stage, NaBH₃CN (1.0 g, 15.4 mmol, 4.0 eq.) was added in portions at 0° C. and stirred for 18 h at room temperature. Upon completion of the reaction, the reaction was diluted with DCM (100 mL). The mixture was washed with water (2×20 mL) and brine (2×20 mL), dried over Na₂SO₄. The resulting mixture was concentrated under reduced pressure and purified by silica gel column chromatography (DCM/methanol: 3:1) to afford compound 5 (0.75 g, 64%) as a white solid.

TLC: EA

R_{f} (Compound 3)=0.4

R_{f} (Compound 5)=0.5

LC-MS: 302.25 [M+1]⁺, 300.25 [M−1]⁻

¹H NMR (400 MHz, CDCl₃): δ 7.10 (d, J=5.9 Hz, 1H), 6.70 (d, J=6.8 Hz, 1H), 5.06 (d, J=10.9 Hz, 1H), 4.19 (d, J=15.7 Hz, 1H), 4.07 (d, J=15.7 Hz, 1H), 3.29 (d, J=24.2 Hz, 1H), 3.07 (s, 2H), 2.75 (s, 2H), 2.25 (s, 1H), 2.09 (s, 1H), 1.57 (d, J=6.1 Hz, 2H), 0.91 (s, 3H).

¹H NMR (400 MHz, DMSO-d₆): δ 9.33 (d, J=1.8 Hz, 1H), 8.47-8.39 (m, 2H), 8.21 (d, J=7.4 Hz, 1H), and 8.14 (d, J=8.4 Hz, 1H).

### General Procedure for the Preparation of Compound 6e

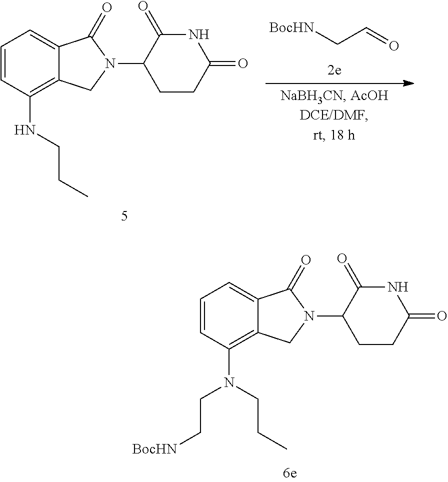 [SMILES: CCCNc1cccc2c1CN(C1CCC(=O)NC1=O)C2=O ; CC(C)(C)OC(=O)NCC=O ; CCCN(CCNC(=O)OC(C)(C)C)c1cccc2c1CN(C1CCC(=O)NC1=O)C2=O]

To a solution of compound 5 (4.6 g, 15.3 mmol, 1.0 eq.) and compound 2e (4.4 g, 27.5 mmol, 1.8 eq.) in DCE/DMF (80 ml, 1/1) was added acetic acid (20 ml) and stirred for 1 h at room temperature under nitrogen atmosphere. NaBH₃CN (3.8 g, 61.1 mmol, 4.0 eq.) was added in portions and stirred for 18 h at room temperature under nitrogen atmosphere. Upon completion of the reaction (as confirmed by TLC analysis, 100% EA and LCMS), the reaction was diluted with DCM (400 mL). The mixture was washed with water (2×40 mL) and brine (2×40 mL), dried over Na₂SO₄. The resulting mixture was concentrated under reduced pressure and purified by silica gel column chromatography (DCM/EA: 2:1) and P-HPLC (acetonitrile/H₂O: 40%-60%) to afford compound 6e (0.7 g, 10%) as a white solid.

TLC: EA=100%

R_{f} (Compound 5)=0.5

R_{f} (Compound 6e)=0.6

LC-MS: 445 [M+1]⁺, 443 [M−1]⁻

¹H NMR (400 MHz, CDCl₃): δ 8.14 (s, 1H), 7.42 (d, J=31.6 Hz, 2H), 7.12 (s, 1H), 5.22 (d, J=10.3 Hz, 1H), 4.45 (s, 2H), 3.48 (s, 1H), 3.17 (d, J=51.0 Hz, 5H), 2.97-2.75 (m, 2H), 2.21 (s, 1H), 1.67 (s, 2H), 1.42 (d, J=17.8 Hz, 14H), 0.87 (s, 3H).

### General Procedure for the Preparation of Compound 49

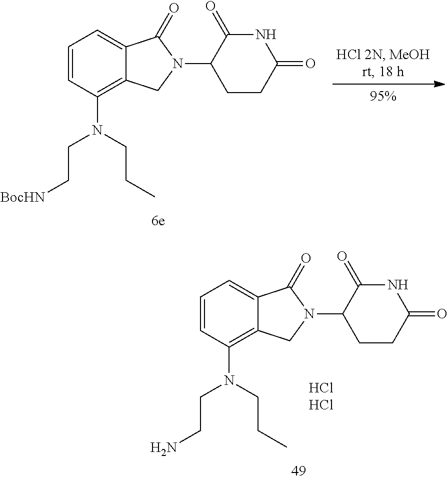 [SMILES: CCCN(CCNC(=O)OC(C)(C)C)c1cccc2c1CN(C1CCC(=O)NC1=O)C2=O ; CCCN(CCN)c1cccc2c1CN(C1CCC(=O)NC1=O)C2=O]

To a solution of compound 6e (0.2 g, 0.4 mmol, 1.0 eq.) in methanol (2 ml) was added HCl/EA (2 mL, 2N, 4.0 mmol, 10.0 eq.). The resulting solution was stirred at room temperature for 18 h. The resulting mixture was concentrated under reduced pressure and purified by P-HPLC (acetonitrile/H₂O: 30%-40%) to afford compound 49 (147 mg, 95%) as a white solid.

LC-MS: 345.35 [M+1]⁺

¹H NMR (400 MHz, D₂O): δ 7.42-7.30 (m, 2H), 7.24 (d, J=7.5 Hz, 1H), 5.00 (dd, J=12.9, 4.4 Hz, 1H), 4.43 (q, J=17.3 Hz, 2H), 3.41 (d, J=5.4 Hz, 2H), 3.06-2.93 (m, 4H), 2.75 (dd, J=25.3, 11.4 Hz, 2H), 2.41 (dd, J=12.7, 4.6 Hz, 1H), 2.17-2.07 (m, 1H), 1.28 (dd, J=14.2, 7.0 Hz, 2H), 0.65 (t, J=7.1 Hz, 3H).

### Compound 50

### N-(4-((2-(2,6-dioxopiperidin-3-yl)-1-oxoisoindolin-4-yl)(propyl)amino)butyl)acetamide

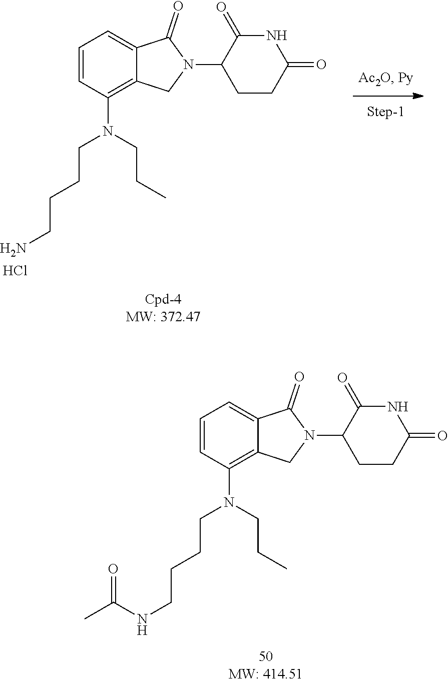 [SMILES: CCCN(CCCCN)c1cccc2c1CN(C1CCC(=O)NC1=O)C2=O ; Cl ; CCCN(CCCCNC(C)=O)c1cccc2c1CN(C1CCC(=O)NC1=O)C2=O]

### Step-1: N-(4-((2-(2,6-dioxopiperidin-3-yl)-1-oxoisoindolin-4-yl)(propyl)amino)butyl)acetamide

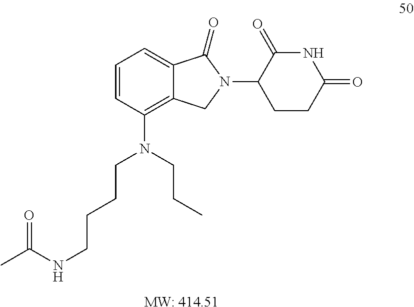 [SMILES: CCCN(CCCCNC(C)=O)c1cccc2c1CN(C1CCC(=O)NC1=O)C2=O ; "MW: 414.51"]

To a solution of 3-(4-((4-aminobutyl)(propyl)amino)-1-oxoisoindolin-2-yl)piperidine-2,6-dione, HCl Compound 4 (200 mg, 0.489 mmol, 1 equiv) in pyridine (3 ml) at 0° C. was added acetic anhydride (0.231 ml, 2.445 mmol, 5 equiv) and was allowed to stir at room temperature for 12 h. Upon completion of reaction (as confirmed by LCMS) the reaction mixture was concentrated under vacuum to give the crude compound, which was purified by reverse-phase column chromatography (Grace column: C18 40 μm, 40 g; flow rate: 20 mL/min; 0.1% aqueous HCOOH/ACN mobile phase) and the pure fractions were lyophilized to give N-(4-((2-(2,6-dioxopiperidin-3-yl)-1-oxoisoindolin-4-yl)(propyl)amino)butyl)acetamide (86 mg, 0.207 mmol, 43.0% yield) as yellow solid.

¹H-NMR (400 MHz, DMSO-d6): δ 10.97 (s, 1H), 7.77 (t, J=5.20 Hz, 1H), 7.38 (t, J=7.60 Hz, 1H), 7.18 (t, J=0.40 Hz, 1H), 7.07 (d, J=7.60 Hz, 1H), 5.10 (q, J=5.20 Hz, 1H), 4.35 (q, J=16.80 Hz, 2H), 3.21-3.13 (m, 4H), 3.00 (q, J=6.40 Hz, 4H), 2.57 (s, 1H), 2.02 (q, J=5.20 Hz, 1H), 1.76 (s, 3H), 1.48-1.36 (m, 6H), 0.84 (t, J=7.60 Hz, 3H).

LCMS: 415.1 (M+H), Method: Mobile phase: A: 0.1% FA in H₂O, Mobile phase: B: ACN, Column: Atlantis dC18 (50×4.6 mm) 5 μm, Flow Rate: 1.5 ml/min Rt (min): 1.622, Area %: 98.693.

HPLC: Method: Mobile phase: A: 0.1% TFA in water, Mobile phase: B: ACN, Column: X-Bridge C8(50×4.6) mm, 3.5 μm Flow: 2.0 mL/min, Rt (min): 1.789, Area %: 99.953.

### Hydrochloride Salt Form of Compound 51

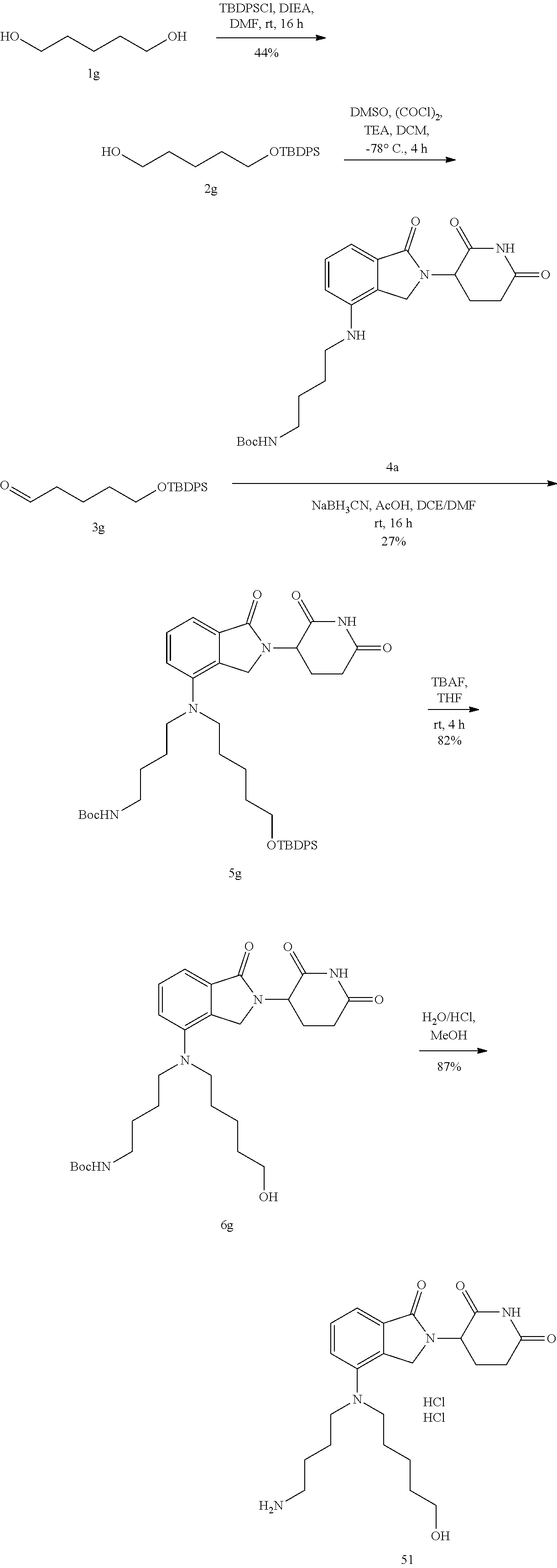

### General Procedure for the Preparation of Compound 2g

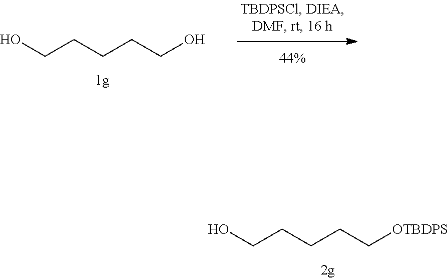

To a solution of compound 1g (5.0 g, 48 mmol, 1 eq.) and tert-Butylchlorodiphenylsilane (13.2 g, 48 mmol, 1.0 eq.) in DMF (50 ml) was added DIEA (18.6 g, 144 mmol, 3.0 eq.) and stirred for 16 h at room temperature under nitrogen atmosphere. Upon completion of the reaction (as confirmed by TLC analysis PE/EA=2:1 and LCMS), the reaction was diluted with DCM (20 mL). The mixture was washed with water (3×20 mL) and brine (20 mL), dried over Na₂SO₄. The resulting mixture was concentrated under reduced pressure and purified by silica gel column chromatography (PE/EA: 10/1) to afford compound 2g (7.2 g, 44%) as a clear oil.

TLC: PE/EA=2/1 KMnO₄

R_{f} (Compound 1g)=0.1

R_{f} (Compound 2g)=0.6

¹H NMR (400 MHz, CDCl₃): δ 7.66 (d, J=5.7 Hz, 4H), 7.38 (d, J=6.8 Hz, 6H), 3.64 (dd, J=17.5, 4.3 Hz, 4H), 1.63-1.50 (m, 5H), 1.43 (d, J=6.3 Hz, 2H), 1.04 (s, 10H).

### General Procedure for the Preparation of Compound 3g

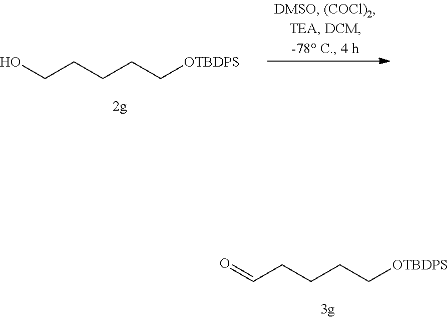

To a solution of Oxalyl chloride (3.2 g, 25.2 mmol, 1.2 eq.) in DCM (40 mL) was added dropwise to a solution of DMSO (7.5 mL, 1.0 V) in DCM (35 mL) at −78° C. over a period of 15 minutes under nitrogen atmosphere and stirred for 15 minutes at the same temperature. The solution of Compound 2g (7.2 g, 20.9 mmol, 1.0 eq.) in DCM (20 mL) was added dropwise over a period of 15 minutes at −78° C. After stirring the reaction mixture for 30 minutes at −78° C., was added TEA (6.35 g, 62.9 mmol, 3.0 eq.) in drops at −78° C. Once addition was complete, the reaction mixture was slowly warmed to 0° C. and stirred for 30 minutes. Upon completion of the reaction (as confirmed by TLC analysis), the reaction mixture was diluted with DCM (50 mL) and washed with 10% citric acid (30 mL), water (50 mL), brine (50 mL) and dried over Na₂SO₄. The solution was filtered and concentrated under vacuum below 30° C. to afford Compound 3g (7.03 g) as clear oil which was used without further purification.

TLC: PE/EA=2/1

R_{f} (Compound 2g)=0.6

R_{f} (Compound 3g)=0.8

¹H NMR (400 MHz, CDCl₃): δ 9.74 (s, 1H), 7.66 (d, J=7.3 Hz, 4H), 7.48-7.33 (m, 6H), 3.67 (t, J=6.1 Hz, 2H), 2.41 (t, J=7.3 Hz, 2H), 1.79-1.68 (m, 2H), 1.60 (dd, J=14.3, 6.6 Hz, 3H), 1.05 (s, 9H).

### General Procedure for the Preparation of Compound 5g

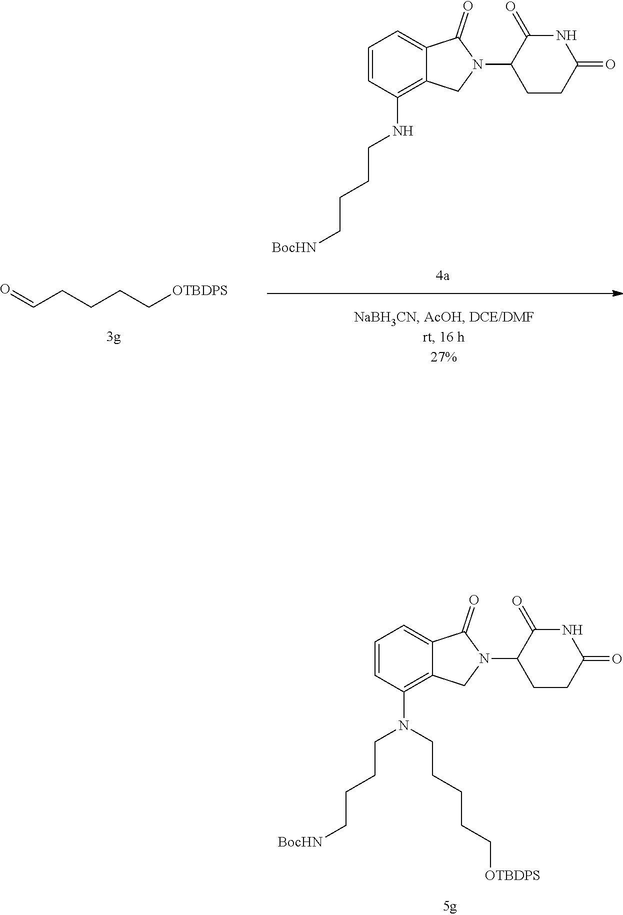

To a solution of compound 4a (2.2 g, 5.1 mmol, 1 eq.) and compound 3g (7.03 g, 20.6 mmol, 4.0 eq.) in DCE/DMF (10 ml) was added acetic acid (2.2 ml), molecular sieve (7.03 g) and stirred for 1 h at room temperature under nitrogen atmosphere. NaBH₃CN (1.6 g, 25.6 mmol, 5 eq.) was added in portions and stirred for 16 h at room temperature under nitrogen atmosphere. Upon completion of the reaction (as confirmed by TLC analysis, 100% EA and LCMS), the reaction was diluted with DCM (20 mL). The mixture was washed with water (3×20 mL) and brine (20 mL), dried over Na₂SO₄. The resulting mixture was concentrated under reduced pressure and purified by silica gel column chromatography (PE/EA: 2/1) and P-HPLC (acetonitrile/H₂O: 40%-60%) to afford compound 5g (1.03 g, 27%) as a white solid.

TLC: EA=100%

R_{f} (Compound 4a)=0.5

R_{f} (Compound 5g)=0.7

LC-MS: 755.60 [M+1]⁺

### General Procedure for the Preparation of Compound 6g

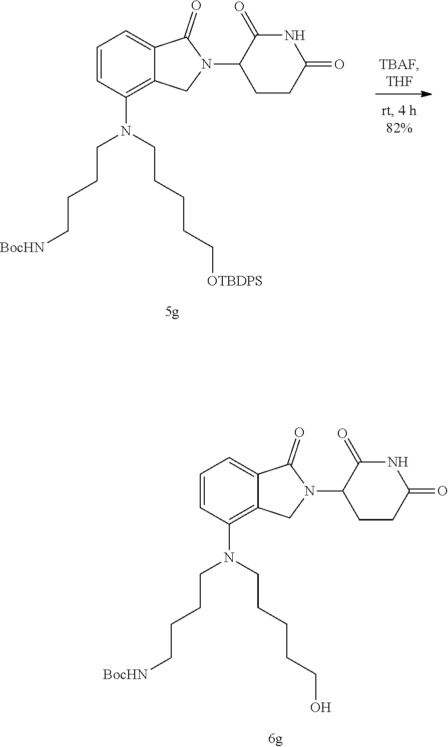

To a solution of compound 5g (1.03 g, 1.37 mmol, 1 eq.) in THF was added TBAF/THF (13.7 mL, 1 N, 13.7 mmol, 10 eq.) and stirred for 4 h at room temperature under nitrogen atmosphere. Upon completion of the reaction (as confirmed by TLC analysis and LCMS), the reaction was diluted with DCM (20 mL). The mixture was washed with water (3×20 mL) and brine (20 mL), dried over Na₂SO₄. The resulting mixture was concentrated under reduced pressure and purified by silica gel column chromatography (PE/EA: 2/1) and P-HPLC (acetonitrile/H₂O: 40%-60%) to afford compound 6g (580 mg, 82%) as a white solid.

TLC: EA=100%

R_{f} (Compound 5g)=0.7

R_{f} (Compound 6g)=0.4

LC-MS: 517.30 [M+1]⁺

### General Procedure for the Preparation of Compound 51

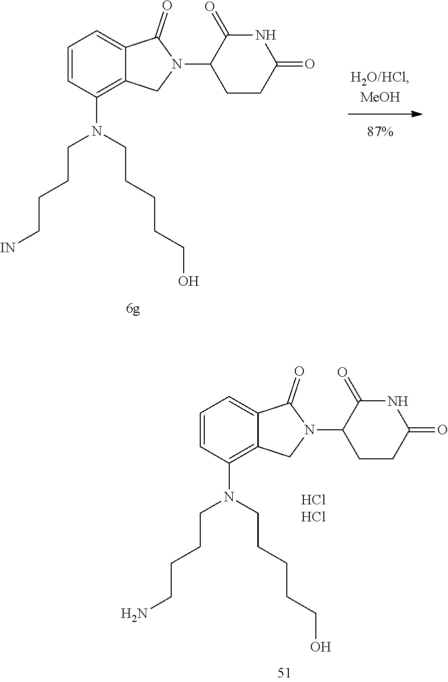

To a solution of compound 6g (200 mg, 0.387 mmol, 1 eq.) in methanol (1.0 ml) was added HCl (2.0 mL, 2N, 4.0 mmol, 10.0 eq.). The resulting solution was stirred at room temperature for 24 h. The reaction was lyophilized to afford compound 51 (164.3 mg, 87%) as a white solid.

TLC: DCM/methanol=10/1

R_{f} (Compound 6g)=0.4

R_{f} (Compound 51)=0.2

LC-MS: 417.40 [M+1]⁺

¹H NMR (400 MHz, D₂O): δ 7.88 (d, J=7.2 Hz, 1H), 7.77 (d, J=7.7 Hz, 1H), 7.70 (t, J=7.6 Hz, 1H), 5.10-5.01 (m, 1H), 4.53 (t, J=13.1 Hz, 3H), 3.56 (d, J=6.9 Hz, 4H), 3.32 (t, J=5.7 Hz, 2H), 2.76 (dd, J=27.9, 6.5 Hz, 4H), 2.47-2.32 (m, 1H), 2.16 (s, 1H), 1.54-1.07 (m, 11H).

### Hydrochloride Salt Form of Compound 52

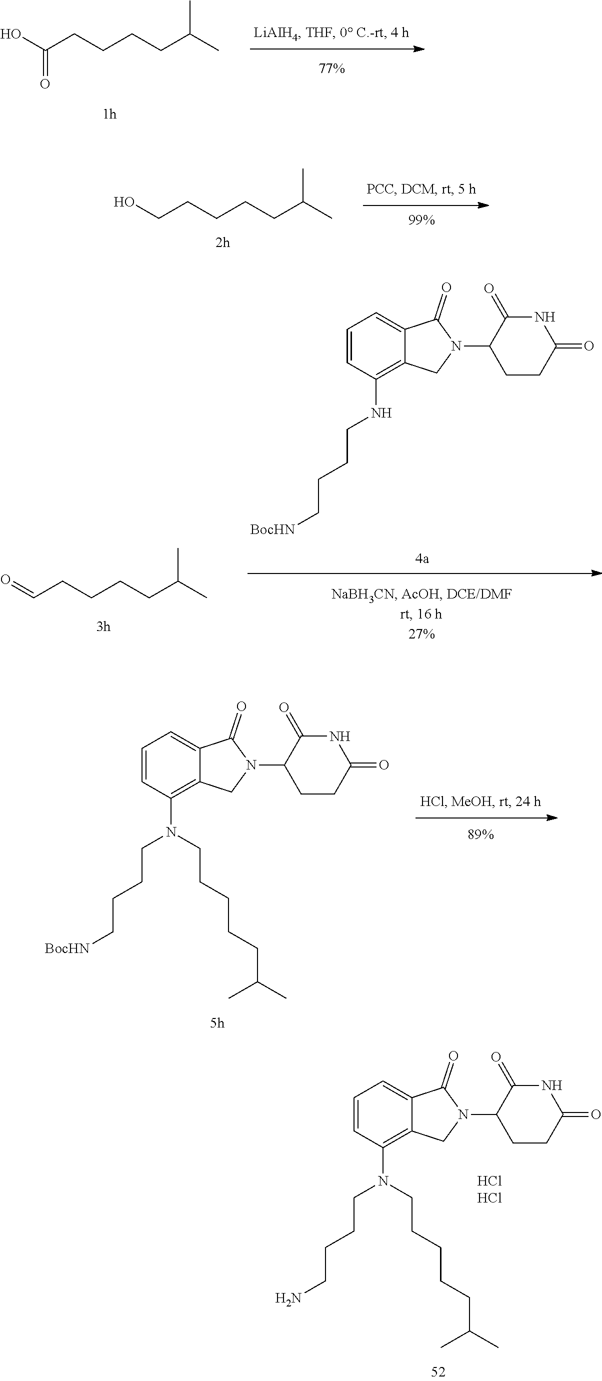

### General Procedure for the Preparation of Compound 2h

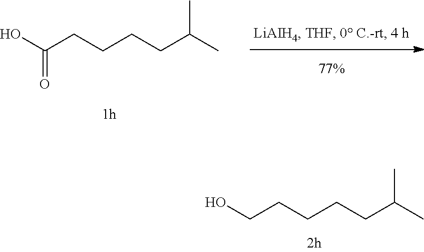

To a solution of Compound 1h (0.5 g, 3.47 mmol, 1.0 eq.) in THF (5 mL) was added dropwise to a solution of LiAlH₄ (145 mg, 3.82 mmol, 1.1 eq.) in THF (5 mL) at 0° C. under nitrogen atmosphere and then stirred for 4 h at room temperature. Upon completion of the reaction (as confirmed by TLC analysis, PE/EA=2/1, Rf 0.6, KMnO₄), the reaction mixture was diluted with MTBE (30 mL) and added Na₂SO₄·10H₂O. The mixture was stirred for 10 min, filtered and concentrated to afford Compound 2h (350 mg, 77%) as clear oil which was used without further purification.

TLC: PE/EA=2:1, KMnO₄

R_{f} (compound 1h)=0.2

R_{f} (compound 2h)=0.6

¹H NMR (400 MHz, CDCl₃): δ 3.62 (t, J=5.7 Hz, 2H), 1.59-1.44 (m, 3H), 1.28 (d, J=15.7 Hz, 5H), 1.17 (s, 3H), 0.85 (d, J=5.6 Hz, 7H).

### General Procedure for the Preparation of Compound 3h

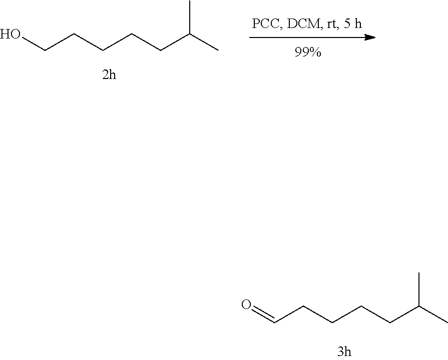

To a solution of compound 2h (600 mg, 4.61 mmol, 1 eq.) in DCM (30 ml) was added PCC (1.99 g, 9.22 mmol, 2.0 eq.) and stirred for 4 h at room temperature. Upon completion of the reaction (as confirmed by TLC analysis PE/EA=3:1), the reaction was added silica gel (4.04 g) and stirred for another 10 minutes. The reaction was filtered and concentrated under reduced pressure below 30° C. to afford compound 3h (714 mg) as clear oil which was used without further purification.

TLC: PE/EA=4/1 KMnO₄

R_{f} (Compound 2h)=0.4

R_{f} (Compound 3h)=0.7

¹H NMR (400 MHz, CDCl₃): δ 9.75 (s, 1H), 2.41 (s, 2H), 1.56 (d, J=29.1 Hz, 3H), 1.24 (d, J=50.5 Hz, 5H), 0.85 (s, 6H).

### General Procedure for the Preparation of Compound 5h

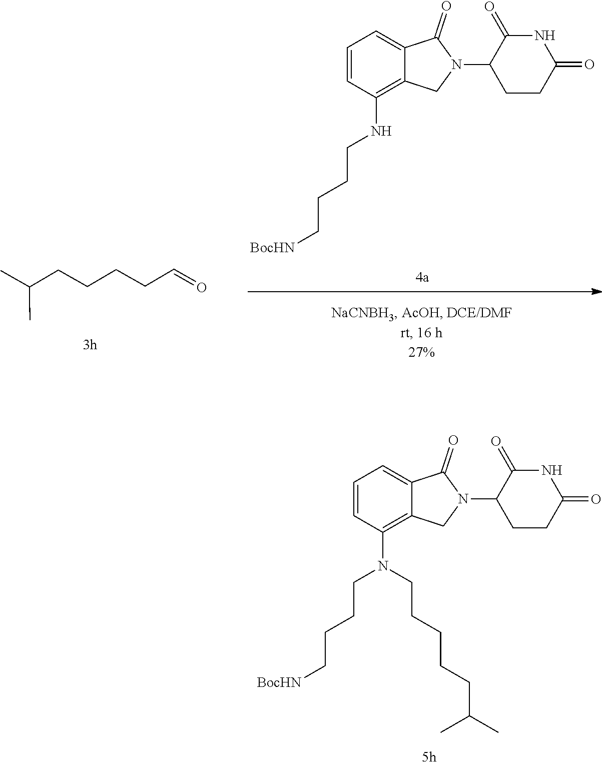

To a solution of Compound 4a (799 mg, 1.86 mmol, 1 eq.) and compound 3h (714 mg, 5.57 mmol, 3.0 eq.) in DCE/DMF (6 ml) was added acetic acid (1 ml), molecular sieve (1.0 g) and stirred for 1 h at room temperature under nitrogen atmosphere. NaBH₃CN (467 mg, 7.43 mmol, 4 eq.) was added in portions and stirred for 16 h at room temperature under nitrogen atmosphere. Upon completion of the reaction (as confirmed by TLC analysis, 100% EA and LCMS), the reaction was diluted with DCM (20 mL). The mixture was washed with water (3×20 mL) and brine (20 mL), dried over Na₂SO₄. The resulting mixture was concentrated under reduced pressure and purified by silica gel column chromatography (DCM/EA: 2/1) and P-HPLC (acetonitrile/H₂O: 40%-60%) to afford compound 5h (275 mg, 27%) as a white solid.

TLC: EA=100%

R_{f} (Compound 4a)=0.4

R_{f} (Compound 5h)=0.6

LC-MS: 543.55 [M+1]⁺

### General Procedure for the Preparation of Compound 52

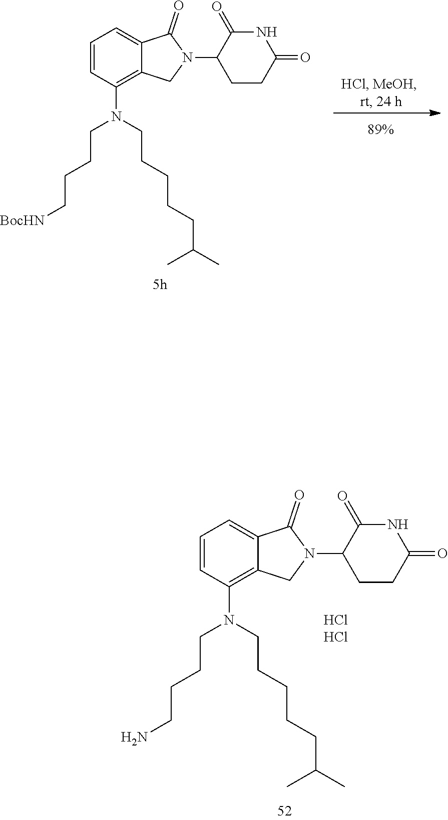 [SMILES: CC(C)CCCCCN(CCCCNC(=O)OC(C)(C)C)c1cccc2c1CN(C1CCC(=O)NC1=O)C2=O ; CC(C)CCCCCN(CCCCN)c1cccc2c1CN(C1CCC(=O)NC1=O)C2=O]

To a solution of compound 5h (200 mg, 0.368 mmol, 1.0 eq.) in methanol (1.0 ml) was added HCl (1.8 mL, 2N, 3.7 mmol, 10.0 eq.). The resulting solution was stirred at room temperature for 24 h. The reaction was lyophilized to afford compound 52 (170.7 mg, 89%) as a white solid.

TLC: DCM/methanol=10/1

R_{f} (Compound 5h)=0.6

R_{f} (Compound 52)=0.1

LC-MS: 443.45 [M+1]⁺

¹H NMR (400 MHz, D₂O): δ 7.84 (d, J=6.9 Hz, 1H), 7.71 (dd, J=21.3, 7.2 Hz, 2H), 5.04 (d, J=9.6 Hz, 1H), 4.51 (t, J=13.0 Hz, 2H), 3.51 (s, 4H), 2.73 (t, J=14.1 Hz, 4H), 2.39 (d, J=9.7 Hz, 1H), 2.14 (s, 1H), 1.31 (dd, J=76.5, 36.4 Hz, 7H), 1.02 (s, 4H), 0.82 (s, 2H), 0.57 (d, J=5.5 Hz, 6H).

### Hydrochloride Salt Form of Compound 53

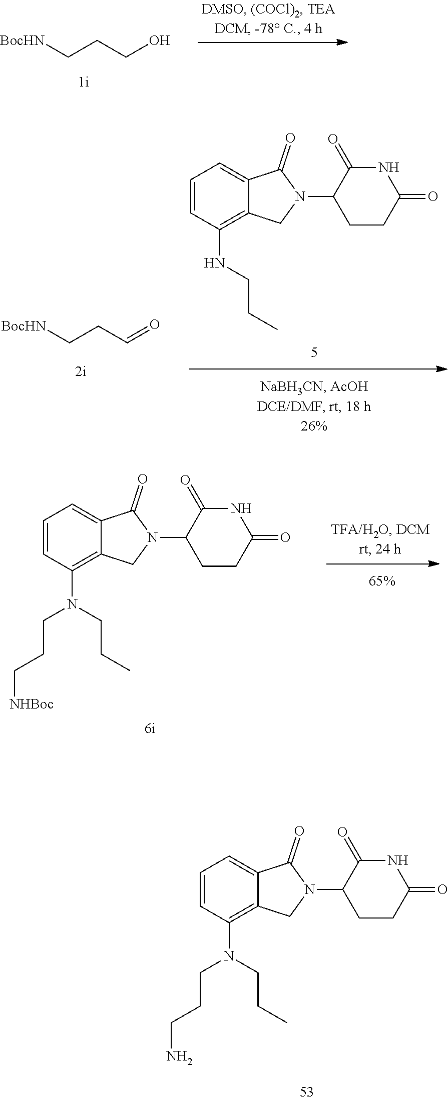 [SMILES: CC(C)(C)OC(=O)NCCCO ; CC(C)(C)OC(=O)NCCC=O ; CCCNc1cccc2c1CN(C1CCC(=O)NC1=O)C2=O ; CCCN(CCCNC(=O)OC(C)(C)C)c1cccc2c1CN(C1CCC(=O)NC1=O)C2=O ; CCCN(CCCN)c1cccc2c1CN(C1CCC(=O)NC1=O)C2=O]

### General Procedure for the Preparation of Compound 2i

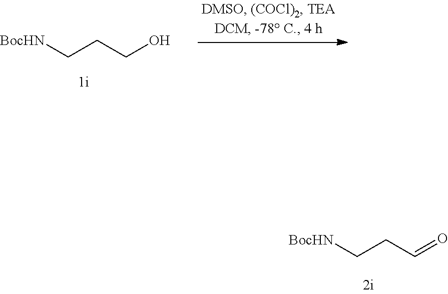 [SMILES: CC(C)(C)OC(=O)NCCCO ; CC(C)(C)OC(=O)NCCC=O]

To a solution of Oxalyl chloride (0.9 g, 6.9 mmol, 1.2 eq.) in DCM (5 mL) was added dropwise to a solution of DMSO (1 mL) in DCM (10 mL) at −78° C. over a period of 15 minutes under nitrogen atmosphere and stirred for 15 minutes at the same temperature. The solution of Compound 1i (1.0 g, 5.7 mmol, 1.0 eq.) in DCM (5 mL) was added dropwise over a period of 15 minutes. After stirring the reaction mixture for 30 minutes at −78° C., was added TEA (1.7 g, 17.1 mmol, 3.0 eq.) in drops at −78° C. Once addition was complete, the reaction mixture was slowly warmed to 0° C. and stirred for 30 minutes. Upon completion of the reaction (as confirmed by TLC analysis, PE/EA=4/1, Rf˜0.4, KMnO₄ or Ninhydrin), the reaction mixture was diluted with DCM (10 mL). The mixture was washed with 10% citric acid (10 mL), water (10 mL) and brine (10 mL), dried over Na₂SO₄, filtered and concentrated under vacuum below 30° C. to afford Compound 2i (0.9 g, crude) as yellow oil which was used without further purification.

TLC: PE/EA=4:1, KMnO₄, Ninhydrin

R_{f} (compound 1i)=0.2

R_{f} (compound 2i)=0.4

LC-MS: No found.

¹H NMR (400 MHz, CDCl₃): δ 9.79 (s, 1H), 4.89 (s, 1H), 3.39 (s, 2H), 2.64 (d, J=35.8 Hz, 2H), 1.40 (s, 10H).

### General Procedure for the Preparation of Compound 6i

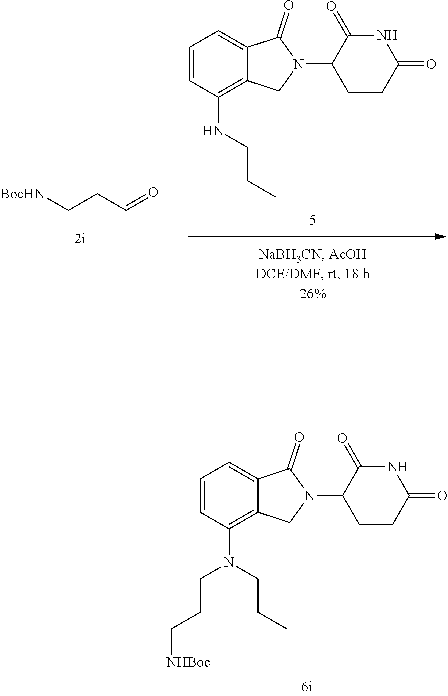 [SMILES: CC(C)(C)OC(=O)NCCC=O ; CCCNc1cccc2c1CN(C1CCC(=O)NC1=O)C2=O ; CCCN(CCCNC(=O)OC(C)(C)C)c1cccc2c1CN(C1CCC(=O)NC1=O)C2=O]

To a solution of compound 2i (2.3 g, 13.3 mmol, 2.0 eq.) and compound 5 (2.0 g, 6.6 mmol, 1.0 eq.) in DCE/DF (1/1, 40 ml) was added acetic acid (10 ml) and stirred for 1 h at room temperature under nitrogen atmosphere. NaBH₃CN (1.7 g, 26.6 mmol, 4.0 eq.) was added in portions and stirred for 18 h at room temperature under nitrogen atmosphere. Upon completion of the reaction (as confirmed by TLC analysis, 100% EA and LCMS), the reaction was diluted with DCM (200 mL). The mixture was washed with water (2×20 mL) and brine (2×20 mL), dried over Na₂SO₄. The resulting mixture was concentrated under reduced pressure and purified by silica gel column chromatography (DCM/methanol: 2/1) and P-HPLC (acetonitrile/H₂O: 40%-60%) to afford compound 6i (0.4 g, 26%) as a white solid.

TLC: EA

R_{f} (Compound 2i)=0.5

R_{f} (Compound 6i)=0.6

LC-MS: 459 [M+1]⁺, 457.30 [M−1]⁻

### General Procedure for the Preparation of Compound 53

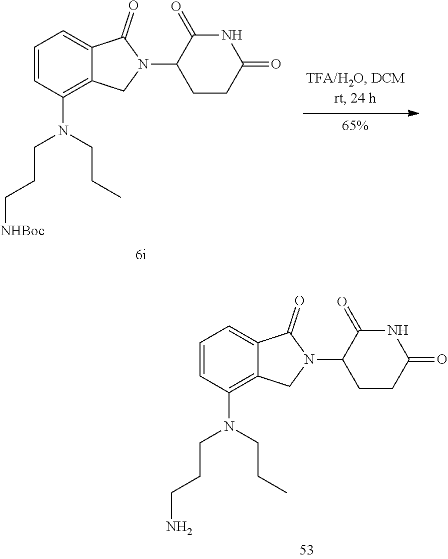 [SMILES: CCCN(CCCNC(=O)OC(C)(C)C)c1cccc2c1CN(C1CCC(=O)NC1=O)C2=O ; CCCN(CCCN)c1cccc2c1CN(C1CCC(=O)NC1=O)C2=O]

To a solution of compound 6i (0.2 g, 0.4 mmol, 1.0 eq.) in DCM/H₂O (1/1, 2 ml) was added TFA (4 mmol, 10.0 eq.). The resulting solution was stirred at room temperature for 24 h. The resulting mixture was concentrated under reduced pressure. The residue was purified by P-HPLC eluted with acetonitrile/H₂O (30%-40%) to afford compound 53 (105 mg, 65%) as a white solid.

LC-MS: 359.30 [M+1]⁺, 357.20 [M−1]⁻

¹H NMR (400 MHz, CD₃OD): δ 7.51-7.36 (m, 2H), 7.29 (d, J=7.5 Hz, 1H), 5.14 (dd, J=12.9, 4.2 Hz, 1H), 4.46 (s, 2H), 3.31 (d, J=9.3 Hz, 5H), 3.14 (d, J=6.5 Hz, 2H), 2.92 (dd, J=19.2, 11.4 Hz, 3H), 2.78 (d, J=17.2 Hz, 1H), 2.54 (d, J=9.8 Hz, 1H), 2.19 (d, J=5.7 Hz, 1H), 1.82 (s, 2H), 1.57-1.44 (m, 2H), 0.88 (t, J=6.8 Hz, 3H).

### Hydrochloride Salt Form of Compound 55

### 3-(4-((4-aminobutyl)(5-aminopentyl)amino)-1-oxoisoindolin-2-yl)piperidine-2,6-dione dihydrochloride

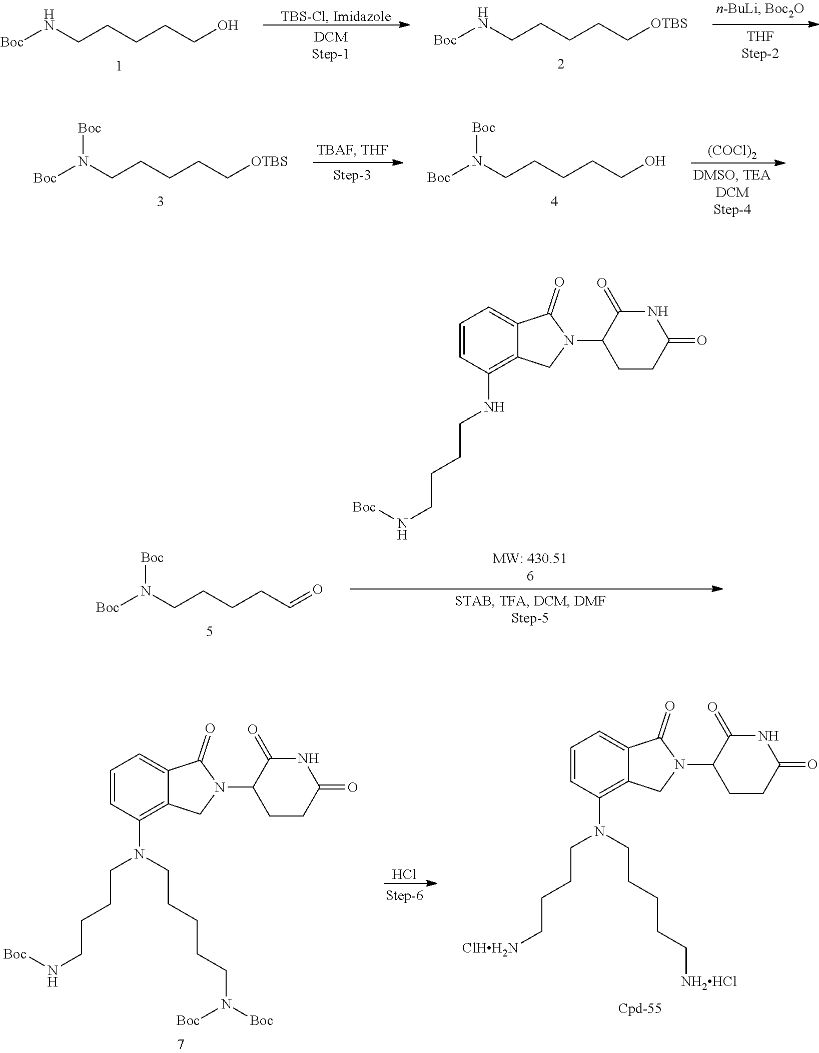

### Step-1: tert-butyl (5-((tert-butyldimethylsilyl)oxy)pentyl)carbamate

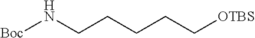 [SMILES: CC(C)(C)OC(=O)NCCCCCO[Si](C)(C)C(C)(C)C]

To a solution of tert-butyl (5-hydroxypentyl)carbamate 1 (1.0 g, 4.92 mmol, 1.0 equiv) in DCM (20 ml) was added imidazole (0.837 g, 12.30 mmol, 2.5 equiv) and TBS-Cl (1.112 g, 7.38 mmol, 1.5 equiv) at 0° C. and was allowed to stir at room temperature for 2 h. Upon completion of the reaction (as confirmed by TLC, 50% EtOAc/pet ether, R_{f}˜0.5, KMnO₄). The reaction mixture was quenched with H₂O (20 ml) and extracted with DCM (2×20 mL). The organic layer was dried over Na₂SO₄ and concentrated under vacuum to give crude of tert-butyl (5-((tert-butyldimethylsilyl)oxy)pentyl)carbamate 2 (1.5 g, 4.70 mmol, 96% yield) as yellow liquid, which was used further without purification. ¹H-NMR (400 MHz, DMSO-d₆): δ 6.74 (s, 1H), 3.54-3.56 (m, 2H), 2.88-2.90 (m, 2H), 1.37-1.43 (m, 6H), 1.156 (s, 9H), 0.86 (s, 9H), 0.03 (s, 6H).

### Step-2: tert-butyl (tert-butoxycarbonyl)(5-((tert-butyldimethylsilyl)oxy)pentyl)carbamate

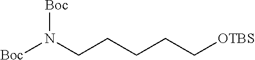 [SMILES: CC(C)(C)OC(=O)N(CCCCCO[Si](C)(C)C(C)(C)C)C(=O)OC(C)(C)C]

To a stirred solution of tert-butyl (5-((tert-butyldimethylsilyl)oxy)pentyl)carbamate 2 (1.5 g, 4.72 mmol, 1.0 equiv) in THF (20 ml)) at 0° C. was added n-BuLi (2.5 M soln. in hexane, 2.267 ml, 5.67 mmol, 1.2 equiv) and the reaction mixture was stirred for 15 min at 0° C. A solution of (Boc)₂O (1.316 ml, 5.67 mmol, 1.2 equiv) in THF (5 ml) was added to the reaction mixture at same temperature. The resulting reaction mixture was warmed to room temperature and stirred for 1 hour. Upon completion of the reaction (as confirmed by TLC analysis, 10% EtOAc/pet ether, R_{f}˜0.7, KMnO₄). The reaction mixture was quenched with water (10 ml) and extracted with DCM (2×50 ml). The combined organic layer was dried over Na₂SO₄ and concentrated under reduced pressure to afford crude of tert-butyl(tert-butoxycarbonyl)(5-((tert-butyldimethylsilyl)oxy) pentyl)carbamate 3 (2.0 g, 4.62 mmol, 98% yield) as a pale yellow liquid, which was used further without purification.

¹H-NMR (400 MHz, DMSO-d₆): δ 3.55-3.58 (m, 2H), 3.44-3.48 (m, 2H), 1.25-1.43 (m, 6H), 1.12 (s, 18H), 0.88 (s, 9H), 0.03 (s, 6H)

### Step-3: tert-butyl (tert-butoxycarbonyl)(5-hydroxypentyl)carbamate

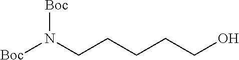 [SMILES: CC(C)(C)OC(=O)N(CCCCCO)C(=O)OC(C)(C)C]

To a stirred solution of the tert-butyl (tert-butoxycarbonyl)(5-((tert-butyldimethylsilyl)oxy)pentyl)carbamate 3 (1.9 g, 4.55 mmol) in THF (20 ml) at room temperature. The resulting solution was stirred at room temperature for over night. Upon completion of the reaction (as confirmed by TLC analysis, 20% EtOAc/pet ether, R_{f}˜0.2, KMnO₄). The reaction mixture was quenched with water (10 ml) and extracted with DCM (2×50 ml). The combined organic layer was dried over Na₂SO₄ and concentrated under reduced pressure to afford the crude as a dark yellow liquid. The crude compound was purified by Isolera chromatography (Biotage R snap cartridge, KP-Sil, 100 g, 100-200 silica gel) using 15-20% EtOAc in pet-ether. The fractions were collected and concentrated under vacuum to afford tert-butyl (tert-butoxycarbonyl)(5-hydroxypentyl)carbamate 4 (1.0 g, 3.27 mmol, 71.9% yield) as a colorless liquid.

¹H-NMR (400 MHz, DMSO-d₆): δ 4.36 (t, J=4.80 Hz, 1H), 3.40-3.47 (m, 2H), 3.37-3.40 (m, 2H), 1.48-1.50 (m, 2H), 1.44 (s, 18H), 1.40-1.42 (m, 2H), 1.25-1.27 (m, 2H),

### Step-4: tert-butyl (tert-butoxycarbonyl)(5-oxopentyl)carbamate

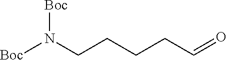 [SMILES: CC(C)(C)OC(=O)N(CCCCC=O)C(=O)OC(C)(C)C]

A solution of oxalyl chloride (0.577 ml, 6.59 mmol, 2.0 equiv) and CH₂Cl₂ (20 ml) was added DMSO (0.936 ml, 13.18 mmol, 4.0 equiv) in CH₂Cl₂ (5 ml) at −78° C. and stirred for 15 minutes at same temperature. To this, a solution of tert-butyl (tert-butoxycarbonyl)(5-hydroxypentyl)carbamate 4 (1.0 g, 3.30 mmol, 1.0 equiv) in CH₂Cl₂ (5 ml) was added at −78° C. After stirring the reaction mixture for 30 minutes at −78° C., triethylamine (2.76 ml, 19.78 mmol, 6.0 equiv) was added in drops at −78° C. Then, the reaction mixture was slowly warmed to 0° C. and stirred for 30 min. Upon completion of the reaction (as confirmed by TLC analysis, 20% EtOAc/pet ether, R_{f}˜0.6, KMn04), the reaction mixture was diluted with DCM (40 mL) and washed with 10% aq citric acid solution (1×40 mL), and water (1×40 mL), dried over Na₂SO₄, filtered and concentrated under vacuum (below 30° C.) to afford of tert-butyl (tert-butoxycarbonyl)(5-oxopentyl)carbamate 5 (0.95 g, 3.08 mmol, 93% yield) as a pale yellow liquid, which was used further without purification.

¹H-NMR (400 MHz, DMSO-d₆): δ 0.00 (s, 1H), 3.47 (m, 2H), 2.42-2.46 (m, 2H), 1.47-1.50 (m, 4H), 1.42 (s, 18H),

### Step-5: tert-butyl (tert-butoxycarbonyl)(5-((4-((tert-butoxycarbonyl)amino)butyl)(2-(2,6-dioxopiperidin-3-yl)-1-oxoisoindolin-4-yl)amino)pentyl)carbamate

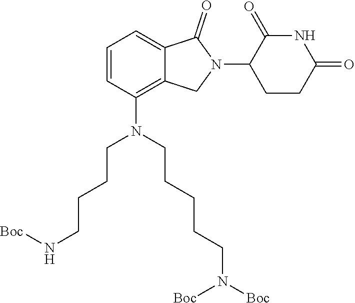 [SMILES: CC(C)(C)OC(=O)NCCCCN(CCCCCN(C(=O)OC(C)(C)C)C(=O)OC(C)(C)C)c1cccc2c1CN(C1CCC(=O)NC1=O)C2=O]

To a stirred mixture of tert-butyl (4-((2-(2,6-dioxopiperidin-3-yl)-1-oxoisoindolin-4-yl)amino)butyl)carbamate 6 (0.7 g, 1.626 mmol, 1.0 equiv) and tert-butyl (tert-butoxycarbonyl)(5-oxopentyl)carbamate 5 (0.980 g, 3.25 mmol, 2.0 equiv) in CH₂Cl₂ (10 ml) and DMF (10 ml) was added trifluoroacetic acid (0.501 ml, 6.50 mmol, 4.0 equiv) and stirred for 10 min at 0° C. Sodium triacetoxyborohydride (1.378 g, 6.50 mmol, 4.0 equiv) was added in portions at 0° C. and the resulted colourless cloudy mass was stirred for 16 h at room temperature. Upon completion of the reaction (as confirmed by TLC analysis, 100% EtOAc, Rf˜0.7), the reaction mixture was quenched with ice cold water (20 ml) and extracted with dichloromethane (2×50 ml). The combined organic layer was dried over anhy. Na₂SO₄ and concentrated under vacuum to afford the crude product as a pale yellow liquid. The crude compound was purified by Isolera chromatography (Biotage R snap cartridge, KP-Sil, 100-200 g, silica gel) using 60-70% of -ethyl acetate in pet-ether to afford tert-butyl(tert-butoxycarbonyl)(5-((4-((tert-butoxycarbonyl)amino) butyl)(2-(2,6-dioxopiperidin-3-yl)-1-oxoisoindolin-4-yl) amino)pentyl)carbamate 7 (0.65 g, 0.891 mmol, 54.8% yield) as a off-white solid.

¹H-NMR (400 MHz, DMSO-d6): δ 0.00 (s, 1H), 7.35-7.39 (m, 1H), 7.18-7.20 (m, 1H), 7.06-7.08 (m, 1H), 6.75-6.78 (m, 1H), 5.08-5.13 (m, 1H), 4.27-4.41 (m, 2H), 3.45-3.46 (m, 2H), 2.89-2.92 (m, 4H), 2.74-2.74 (m, 4H), 2.51-2.52 (m, 2H), 2.50-2.50 (m, 1H), 2.00-2.03 (m, 1H), 1.45-1.49 (m, 15H), 1.42 (s, 18H), 1.20-1.26 (m, 2H),

LCMS: 616.4 (M-Boc). Method: Atlantis dC18 (50×4.6 mm) 5 μm, Mobile phase: A: 0.1% FA in H₂O, Mobile phase: B: CAN, Flow Rate: 1.5 ml/min

HPLC: Method: Mobile Phase A: 0.1% TFA in water, Mobile Phase B: Acetonitrile, Flow rate:2.0 ml/min. Column: X-Bridge C8(50×4.6) mm, 3.5 μm. RT: 4.972 min, Area:98.095%

### Step-6: 3-(4-((4-aminobutyl)(5-aminopentyl)amino)-1-oxoisoindolin-2-yl)piperidine-2,6-dione dihydrochloride

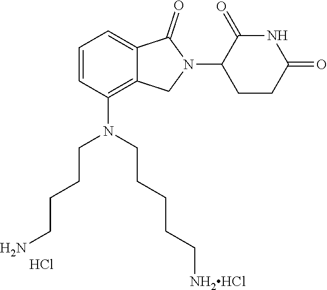

To a stirred solution of tert-butyl(tert-butoxycarbonyl)(5-((4-((tert-butoxycarbonyl)amino) butyl)(2-(2,6-dioxopiperidin-3-yl)-1-oxoisoindolin-4-yl)amino)pentyl) carbamate 7 (0.35 g, 0.489 mmol, 1.0 equiv) in dichloromethane (5 ml) was added HCl (4M soln. in ethylacetate, 10 ml) at 0° C. and then stirred at room temperature for 3 h. The progress of the reaction was monitored by LCMS. Reaction mixture was concentrated under vacuum at below 40° C., to afford pale yellow solid. The obtained solid was dissolved in water (10 ml) and washed with MTBE (2×10 ml), the water layer was lyophilized to afford 3-(4-((4-aminobutyl)(5-aminopentyl)amino)-1-oxoisoindolin-2-yl)piperidine-2,6-dione, 2HCl (0.2 g, 0.404 mmol, 83% yield) as a pale yellow solid.

¹H-NMR (400 MHz, CD₃OD): δ 8.20 (s, 2H), 7.83 (s, 1H), 5.22-5.27 (m, 1H), 4.84-4.88 (m, 2H), 3.72 (m, 4H), 2.87-2.97 (m, 6H), 2.63-2.66 (m, 1H), 2.27-2.28 (m, 1H), 1.60-1.70 (m, 7H), 1.42-1.47 (m, 3H).

LCMS: 416.3 (M+H). Method: Column: XBridge C8 (50×4.6 mm) 3.5 μm, Mobile phase: A: 0.1% TFA in H₂O, Mobile phase: B: 0.1% TFA in Acetonitrile, Flow Rate: 1.5 ml/min.

HPLC: Method: Mobile Phase A: 0.1% TFA in water, Mobile Phase B: Methanol, Flow rate: 0.7 ml/min. Column: Atlantis dC18 (250×4.6) mm, 5 μm. RT: 12.223 min, Area: 98.472%.

### Hydrochloride Salt Form of Compound 56

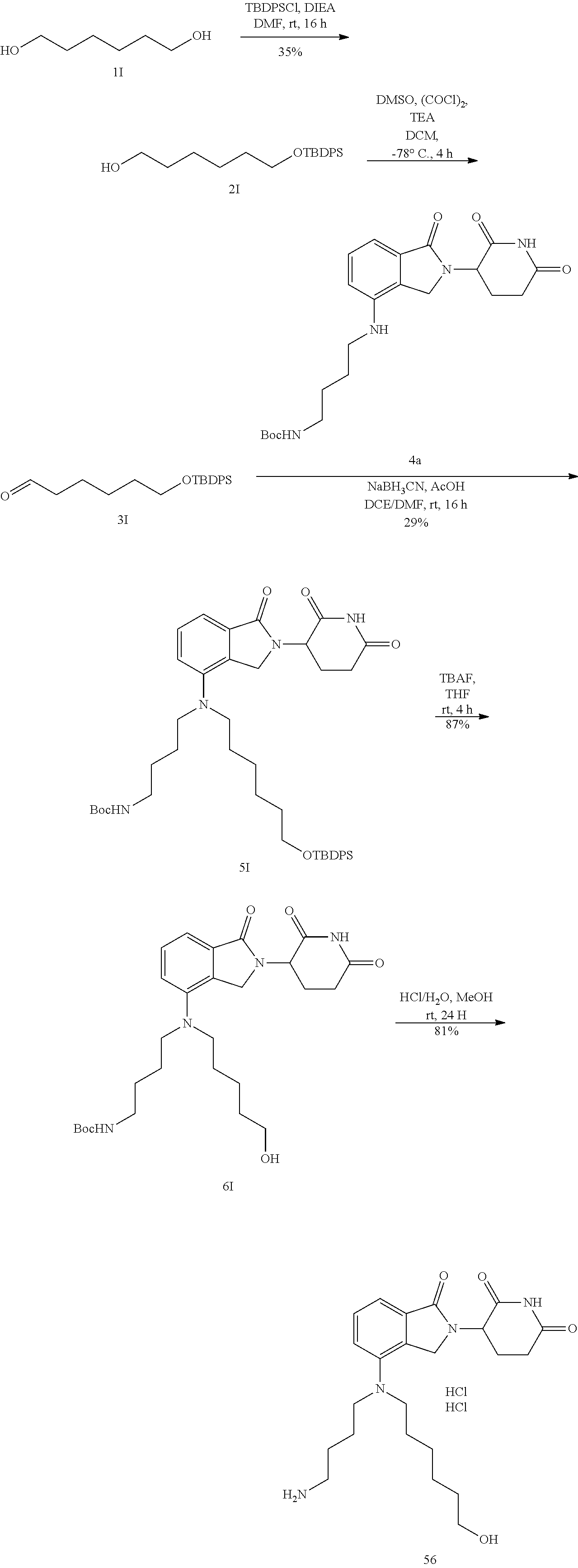

### General Procedure for the Preparation of Compound 2l

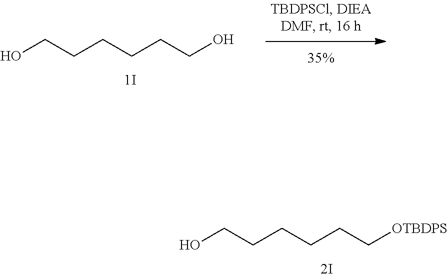

To a solution of compound 1l (10 g, 85.6 mmol, 1 eq.) and tert-Butylchlorodiphenylsilane (23.25 g, 85.6 mmol, 1.0 eq.) in DMF (100 ml) was added DIEA (32.7 g, 254 mmol, 3.0 eq.) and stirred for 16 h at room temperature under nitrogen atmosphere. Upon completion of the reaction (as confirmed by TLC analysis PE/EA=2: 1 and LCMS), the reaction was diluted with DCM (20 mL) and washed water (3×20 mL) and brine (20 mL), dried over Na₂SO₄, filtered. The resulting mixture was concentrated under reduced pressure and purified by silica gel column chromatography (PE/EA: 10/1) to afford compound 2l KMnO₄

R_{f} (Compound 1l)=0.1

R_{f} (Compound 2l)=0.6

¹H NMR (400 MHz, CDCl₃): δ 7.67 (d, J=5.6 Hz, 4H), 7.39 (d, J=6.8 Hz, 6H), 3.72-3.56 (m, 4H), 1.58 (t, J=14.0 Hz, 5H), 1.31 (d, J=37.5 Hz, 6H), 1.05 (s, 9H).

### General Procedure for the Preparation of Compound 3l

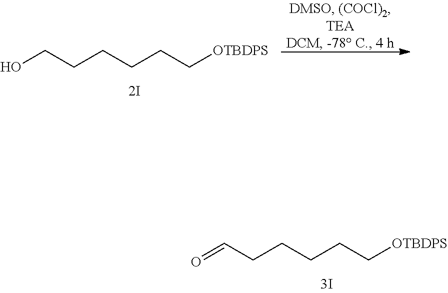

To a solution of Oxalyl chloride (2.14 g, 16.8 mmol, 1.2 eq.) in DCM (20 mL) was added dropwise to a solution of DMSO (5 mL, 1.0 V) in DCM (20 mL) at −78° C. over a period of 15 minutes under nitrogen atmosphere and stirred for 15 minutes at the same temperature. The solution of Compound 2l (5.0 g, 14.02 mmol, 1.0 eq.) in DCM (10 mL) was added dropwise over a period of 15 minutes. After stirring the reaction mixture for 30 minutes at −78° C., was added TEA (4.3 g, 42.1 mmol, 3.0 eq.) in drops at −78° C. Once addition was complete, the reaction mixture was slowly warmed to 0° C. and stirred for 30 minutes. Upon completion of the reaction (as confirmed by TLC analysis), the reaction mixture was diluted with DCM (50 mL). The mixture was washed with 10% citric acid (30 mL), water (50 mL) and brine (50 mL), dried over Na₂SO₄, filtered and concentrated under vacuum below 30° C. to afford Compound 3l (4.9 g) as clear oil which was used without further purification.

TLC: PE/EA=2/1

R_{f} (Compound 2l)=0.6

R_{f} (Compound 3l)=0.8

### General Procedure for the Preparation of Compound 5l

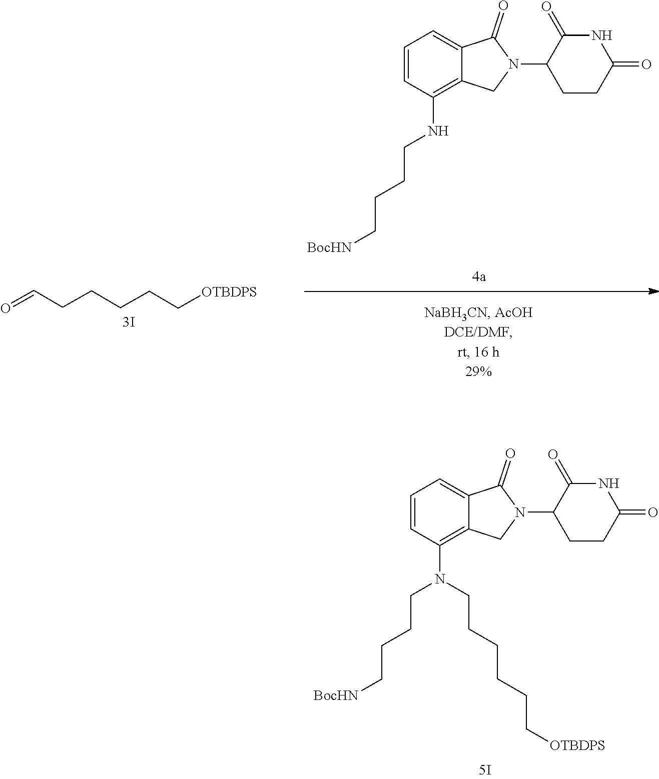

To a solution of compound 4a (1.5 g, 3.45 mmol, 1 eq.) and compound 31 (4.9 g, 13.8 mmol, 4.0 eq.) in DCE/DMF (15 ml) was added acetic acid (1.5 ml), molecular sieve (5.0 g) and stirred for 1 h at room temperature under nitrogen atmosphere. NaBH₃CN (1.08 g, 17.2 mmol, 5 eq.) was added in portions and stirred for 16 h at room temperature under nitrogen atmosphere. Upon completion of the reaction (as confirmed by TLC analysis, 100% EA and LCMS), the reaction was diluted with DCM (20 mL). The mixture was washed with water (3×20 mL) and brine (20 mL), dried over Na₂SO₄. The resulting mixture was concentrated under reduced pressure and purified by silica gel column chromatography (PE/EA: 2/1) and P-HPLC (acetonitrile/H₂O: 40%-60%) to afford compound 51 (1.03 g, 29%) as a white solid.

TLC: EA=100%

R_{f} (Compound 4a)=0.5

R_{f} (Compound 51)=0.7

LC-MS: 769.60 [M+1]⁺

### General Procedure for the Preparation of Compound 6l

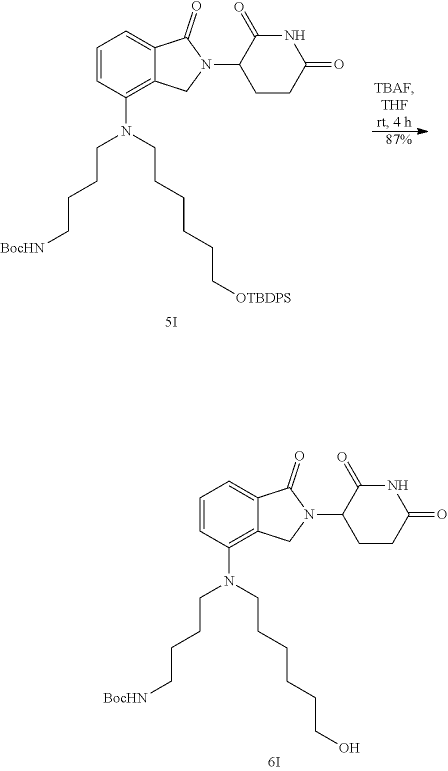

To a solution of compound 5l (1.03 g, 1.34 mmol, 1 eq.) in THF was added TBAF/THF (13.4 mL, 1 N, 13.4 mmol, 10 eq.) and stirred for 4 h at room temperature under nitrogen atmosphere. Upon completion of the reaction (as confirmed by TLC analysis and LCMS), the reaction was diluted with DCM (20 mL). The mixture was washed with water (3×20 mL) and brine (20 mL), dried over Na₂SO₄. The resulting mixture was concentrated under reduced pressure and purified by silica gel column chromatography (PE/EA: 2/1) and P-HPLC (acetonitrile/H₂O: 40%-60%) to afford compound 61 (620 mg, 87%) as a white solid.

TLC: EA=100%

R_{f} (Compound 51)=0.7

R_{f} (Compound 61)=0.4

LC-MS: 531.45 [M+1]⁺

### General Procedure for the Preparation of Compound 56

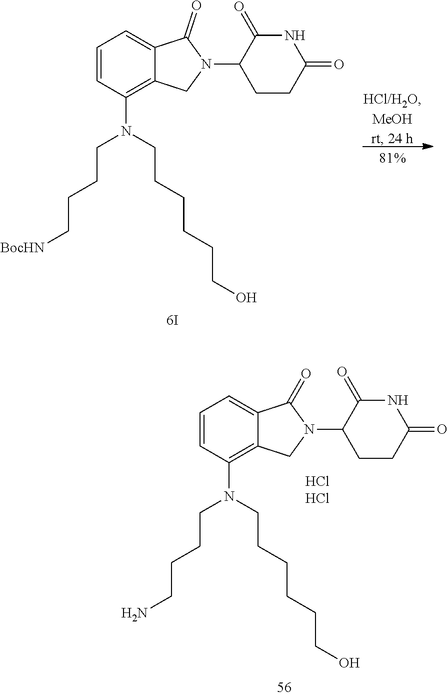 [SMILES: CC(C)(C)OC(=O)NCCCCN(CCCCCCO)c1cccc2c1CN(C1CCC(=O)NC1=O)C2=O ; NCCCCN(CCCCCCO)c1cccc2c1CN(C1CCC(=O)NC1=O)C2=O]

To a solution of compound 6l (300 mg, 0.566 mmol, 1 eq.) in methanol (1 ml) was added HCl (2.8 mL, 2N, 5.6 mmol, 10 eq.). The resulting solution was stirred at room temperature for 24 h. The reaction was lyophilized to afford compound 56 (231 mg, 81%) as a white solid.

TLC: DCM/methanol=10/1

R_{f} (Compound 15-A)=0.4

R_{f} (Compound 56)=0.2

LC-MS: 431.40 [M+1]⁺

¹H NMR (400 MHz, D₂O): δ 7.88 (d, J=7.0 Hz, 1H), 7.74 (dd, J=22.2, 7.6 Hz, 2H), 5.07 (d, J=8.9 Hz, 1H), 4.54 (t, J=13.1 Hz, 2H), 3.55 (d, J=5.1 Hz, 4H), 3.34 (s, 2H), 2.87-2.67 (m, 4H), 2.41 (d, J=8.3 Hz, 1H), 2.17 (s, 1H), 1.35 (dd, J=63.6, 23.7 Hz, 9H), 1.10 (s, 4H).

### Hydrochloride Salt Form of Compound 57

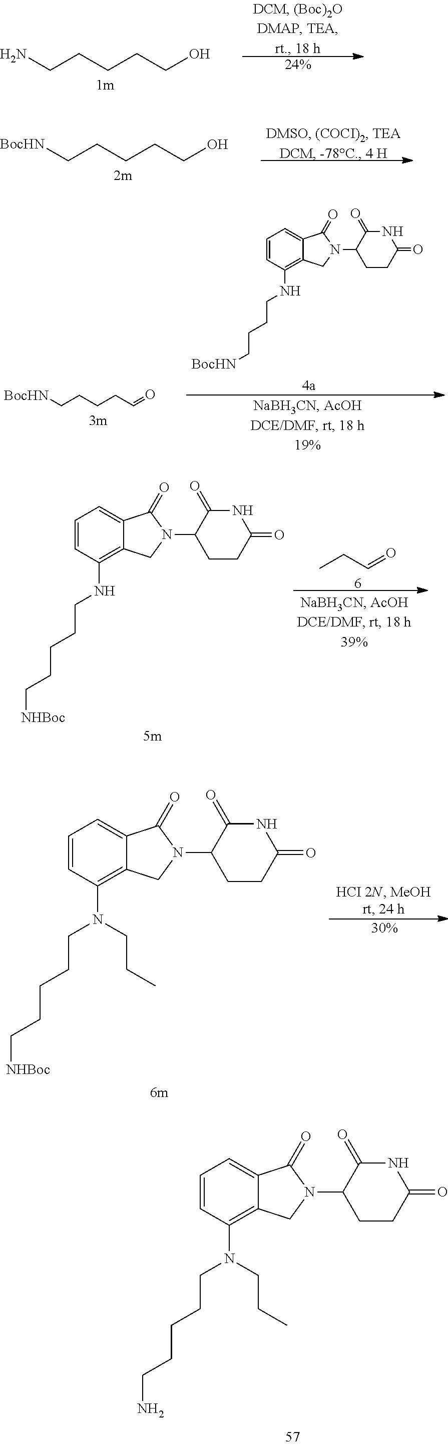

### General Procedure for the Preparation of Compound 2m

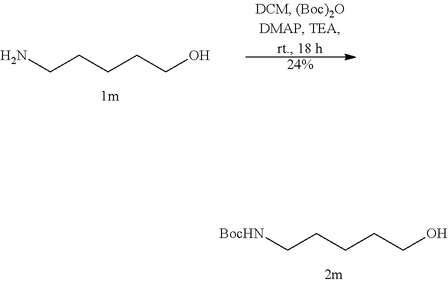 [SMILES: NCCCCCO ; CC(C)(C)OC(=O)NCCCCCO]

To a solution of 1m (14.3 g, 138.6 mmol, 1.0 eq.) and TEA (42.0 g, 415.8 mmol, 3.0 eq.) in DCM (150 mL) was added dropwise to a solution of (Boc)₂O (36.3 g, 166.3 mmol, 1.2 eq.), then DMAP (1.7 g, 13.9 mmol, 0.1 eq.) was added and stirred at room temperature for 18 h. The reaction mixture was diluted with DCM (300 mL) and washed with brine (3×50 mL), dried over Na₂SO₄, filtered and concentrated under vacuum to afford Compound 2m (7.1 g, 24%) as colorless oil which was used further.

TLC: PE/EA=4:1, KMnO₄, Ninhydrin

R_{f} (compound 1m)=0.2

R_{f} (compound 2m)=0.4

LC-MS: No found.

### General Procedure for the Preparation of Compound 3m

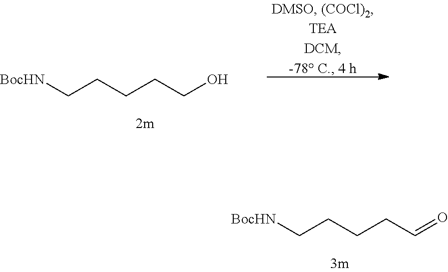 [SMILES: CC(C)(C)OC(=O)NCCCCCO ; CC(C)(C)OC(=O)NCCCCC=O]

To a solution of Oxalyl chloride (5.4 g, 42.4 mmol, 1.2 eq.) in DCM (35 mL) was added dropwise to a solution of DMSO (7 mL, 1.0 V) in DCM (70 mL) at −78° C. over a period of 15 minutes under nitrogen atmosphere and stirred for 15 minutes at the same temperature. The solution of Compound 2m (7.1 g, 35.3 mmol, 1.0 eq.) in DCM (35 mL) was added dropwise over a period of 15 minutes. After stirring the reaction mixture for 30 minutes at −78° C., was added TEA (10.7 g, 105.9 mmol, 3.0 eq.) in drops at −78° C. Once addition was complete, the reaction mixture was slowly warmed to 0° C. and stirred for 30 minutes. Upon completion of the reaction (as confirmed by TLC analysis, PE/EA=4/1, R_{f}˜0.4, KMnO₄ or Ninhydrin), the reaction mixture was diluted with DCM (70 mL). The mixture was washed with 10% citric acid solution (35 mL), water (35 mL) and brine (35 mL), dried over Na₂SO₄. The result mixture was filtered and concentrated under vacuum below 30° C. to afford Compound 3m (7.4 g) as yellow oil which was used further without purification.

TLC: PE/EA=4:1, KMnO₄, Ninhydrin

R_{f} (compound 2m)=0.2

R_{f} (compound 3m)=0.4

LC-MS: No found.

### General Procedure for the Preparation of Compound 5m

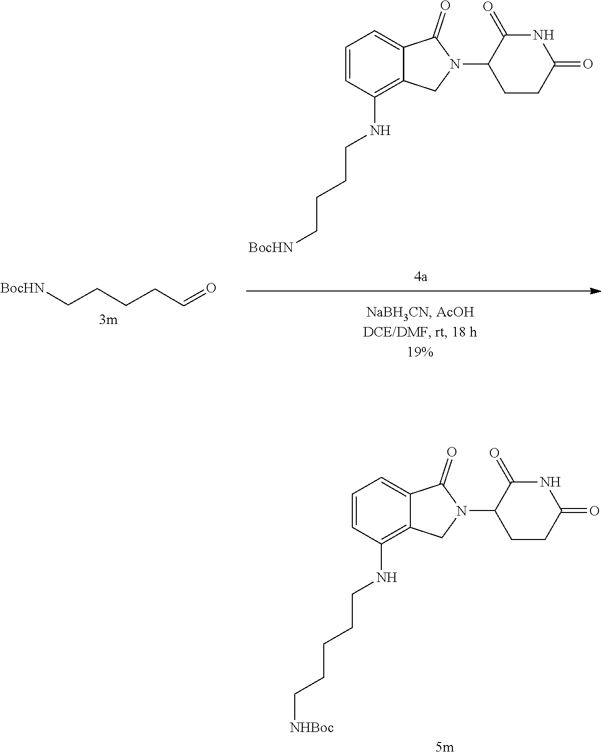 [SMILES: CC(C)(C)OC(=O)NCCCCNc1cccc2c1CN(C1CCC(=O)NC1=O)C2=O ; CC(C)(C)OC(=O)NCCCCC=O ; CC(C)(C)OC(=O)NCCCCCNc1cccc2c1CN(C1CCC(=O)NC1=O)C2=O]

To a solution of compound 3m (7.8 g, 41.7 mmol, 1.8 eq.) and compound 4a (6.0 g, 23.1 mmol, 1.0 eq.) in DCE/DMF (1/1, 120 ml) was added acetic acid (30 ml). The reaction was stirred for 2 h at room temperature. At this stage, NaBH₃CN (5.8 g, 92.6 mmol, 4.0 eq.) was added in portions at 0° C. and stirred for 18 h at room temperature. Upon completion of the reaction, the reaction was diluted with DCM (600 mL). The mixture was washed with water (2×60 mL) and brine (2×60 mL), dried over Na₂SO₄. The resulting mixture was concentrated under reduced pressure and purified by silica gel column chromatography (DCM/EA: 3/1) to afford compound 5m (2.0 g, 19%) as a white solid.

TLC: EA

R_{f} (Compound 3m)=0.4

R_{f} (Compound 5m)=0.6

LC-MS: 445.35 [M+1]⁺, 443.30 [M−1]⁻

### General Procedure for the Preparation of Compound 6m

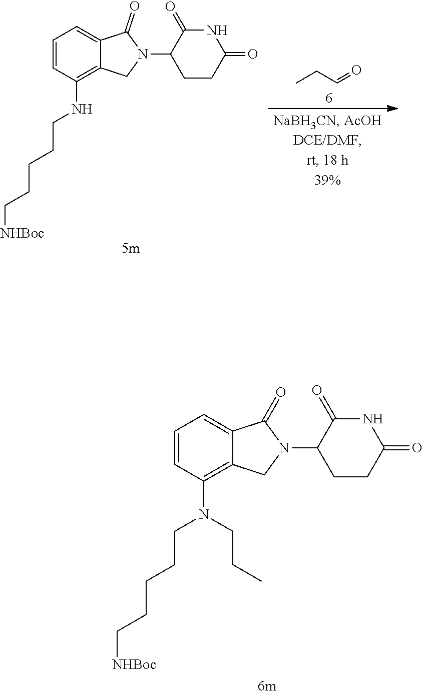

To a solution of compound 5m (2.0 g, 4.5 mmol, 1.0 eq.) and compound 6 (2.6 g, 45 mmol, 10.0 eq.) in DCE/DMF (40 ml, 1/1) was added acetic acid (10 inland stirred for 1 h at room temperature under nitrogen atmosphere. NaBH₃CN (1.1 g, 18 mmol, 4.0 eq.) was added in portions and stirred for 18 h at room temperature under nitrogen atmosphere. Upon completion of the reaction (as confirmed by TLC analysis, 100% EA and LCMS), the reaction was diluted with DCM (200 mL). The mixture was washed with water (2×40 mL) and brine (2×40 mL), dried over Na₂SO₄. The resulting mixture was concentrated under reduced pressure and purified by silica gel column chromatography (eluted with DCM/EA: 4:1) and P-HPLC (acetonitrile/H₂O: 40%-60%) to afford compound 6m (0.87 g, 39%) as a white solid.

TLC: EA=100%

R_{f} (Compound 5m)=0.5

R_{f} (Compound 6m)=0.65

LC-MS: 487.40 [M+1]⁺, 485.40 [M−1]⁻

### General Procedure for the Preparation of Compound 57

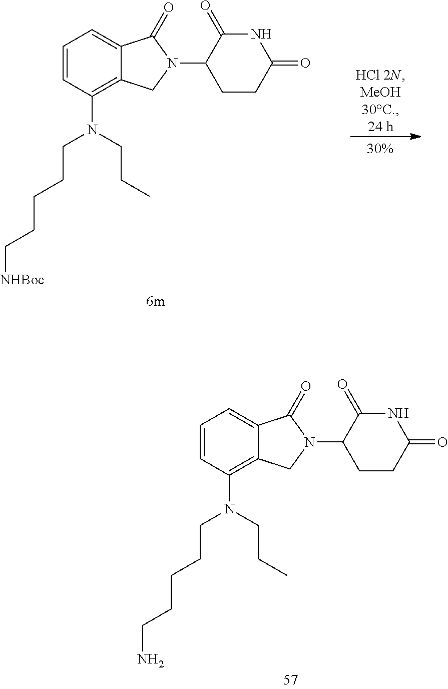 [SMILES: CCCN(CCCCCNC(=O)OC(C)(C)C)c1cccc2c1CN(C1CCC(=O)NC1=O)C2=O ; CCCN(CCCCCN)c1cccc2c1CN(C1CCC(=O)NC1=O)C2=O]

To a solution of compound 6m (870 mg, 1.8 mmol, 1.0 eq.) in methanol (4 ml) was added HCl/EA (9 mL, 2 N, 18 mmol, 10.0 eq.). The resulting solution was stirred 30° C. for 18 h. The resulting mixture was concentrated under reduced pressure. The residue was purified by P-HPLC (acetonitrile/H₂O: 30%-40%) to afford compound 57 (144 mg, 30%) as a white solid.

LC-MS: 387.40 [M+1]⁺, 385.15 [M−1]⁻

¹H NMR (400 MHz, CD₃OD): δ 7.59 (s, 3H), 5.18 (d, J=10.0 Hz, 1H), 4.57 (s, 2H), 3.47 (s, 3H), 3.00-2.74 (m, 5H), 2.55 (d, J=10.6 Hz, 1H), 2.21 (s, 1H), 1.73-1.45 (m, 7H), 1.39 (d, J=6.4 Hz, 3H), 0.90 (t, J=6.3 Hz, 4H).

### Hydrochloride Salt Form of Compound 58

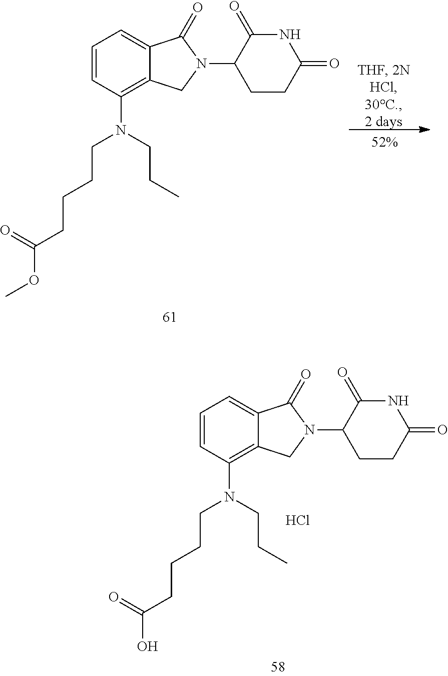 [SMILES: CCCN(CCCCC(=O)OC)c1cccc2c1CN(C1CCC(=O)NC1=O)C2=O ; CCCN(CCCCC(=O)O)c1cccc2c1CN(C1CCC(=O)NC1=O)C2=O ; Cl]

To a solution of compound 61 (0.2 g, 0.5 mmol, 1.0 eq.) in THF (2 ml) was added HCl/EA (2.4 mL, 2 N, 4.8 mmol, 10.0 eq.) and stirred at 30° C. for 2 days. The resulting mixture was concentrated under reduced pressure and purified by P-HPLC (acetonitrile/H₂O: 30%-40%) to afford compound 58 (102 mg, 52%) as a white solid.

LC-MS: 402.30 [M+1]⁺, 400.20 [M−1]⁻

¹H NMR (400 MHz, DMSO-d₆): δ 10.99 (s, 1H), 7.25 (s, 1H), 6.90 (d, J=4.0 Hz, 1H), 6.73 (d, J=4.0 Hz, 1H), 5.57 (s, 1H), 5.59 (d, J=12.0 Hz, 1H), 4.26-3.85 (m, 2H), 3.05 (s, 2H), 3.85-3.75 (m, 1H), 2.70-2.50 (m, 1H), 2.31-2.20 (m, 1H), 1.99 (s, 1H), 1.55 (s, 2H), 0.89 (s, 3H).

### Hydrochloride Salt Form of Compound 60

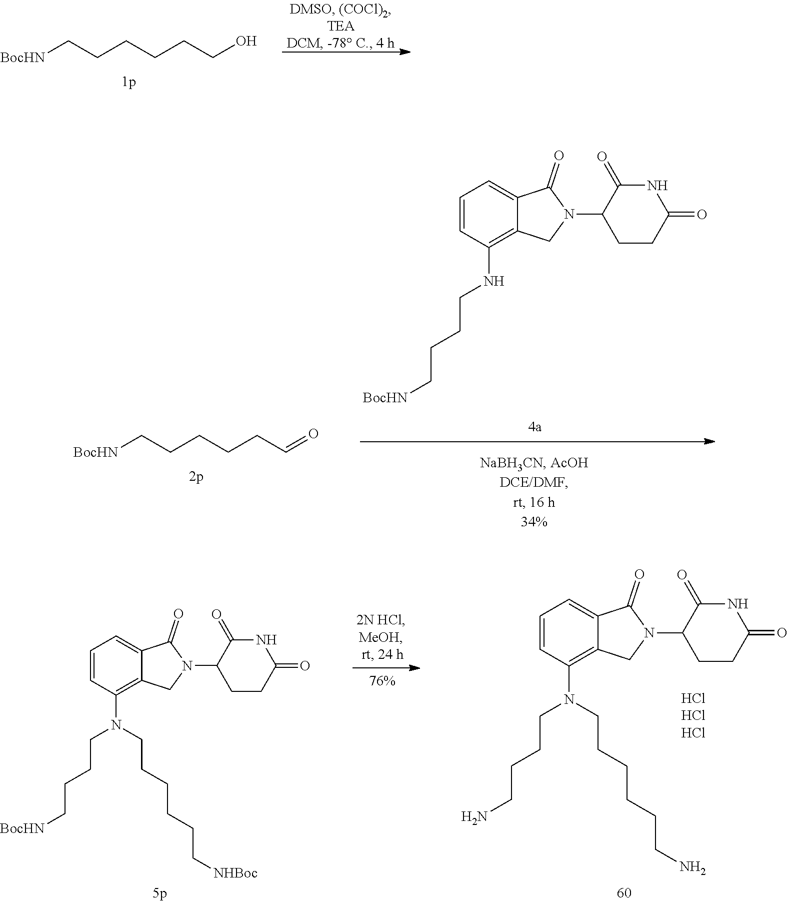 [SMILES: CC(C)(C)OC(=O)NCCCCCCO ; CC(C)(C)OC(=O)NCCCCNc1cccc2c1CN(C1CCC(=O)NC1=O)C2=O ; CC(C)(C)OC(=O)NCCCCCC=O ; CC(C)(C)OC(=O)NCCCCCCN(CCCCNC(=O)OC(C)(C)C)c1cccc2c1CN(C1CCC(=O)NC1=O)C2=O ; NCCCCCCN(CCCCN)c1cccc2c1CN(C1CCC(=O)NC1=O)C2=O]

### General Procedure for the Preparation of Compound 2p

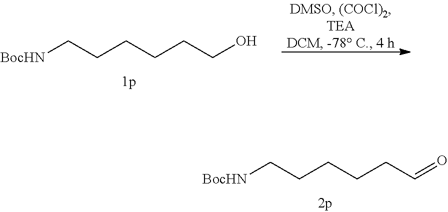 [SMILES: CC(C)(C)OC(=O)NCCCCCCO ; CC(C)(C)OC(=O)NCCCCCC=O]

To a solution of Oxalyl chloride (2.9 g, 22.85 mmol, 1.2 eq.) in DCM (10 mL) was added dropwise to a solution of DMSO (4.2 mL, 1.0 V) in DCM (20 mL) at −78° C. over a period of 15 minutes under nitrogen atmosphere and stirred for 15 minutes at the same temperature. The solution of Compound 1p (4.2 g, 19.32 mmol, 1.0 eq.) in DCM (10 mL) was added dropwise over a period of 15 minutes. After stirring the reaction mixture for 30 minutes at −78° C., was added TEA (5.8 g, 57.98 mmol, 3.0 eq.) in drops at −78° C. Once addition was complete, the reaction mixture was slowly warmed to 0° C. and stirred for 30 minutes. Upon completion of the reaction (as confirmed by TLC analysis, PE/EA=4/1, R_{f}˜0.6, KMnO₄ or Ninhydrin), the reaction mixture was diluted with DCM (50 mL). The mixture was washed with 10% citric acid (30 mL), water (50 mL) and brine (50 mL), dried over Na₂SO₄, filtered and concentrated under vacuum below 30° C. to afford Compound 2p (4.1 g, crude) as yellow oil which was used without further purification.

TLC: PE/EA=2:1, KMnO₄, Ninhydrin

R_{f} (compound 1p)=0.2

R_{f} (compound 2p)=0.6

LC-MS: No found.

¹H NMR (400 MHz, DMSO-d₆): δ 9.63 (s, 1H), 6.75 (s, 1H), 2.86 (d, J=5.2 Hz, 2H), 2.38 (s, 2H), 1.56-1.42 (m, 2H), 1.34 (s, 11H), 1.20 (d, J=5.4 Hz, 3H).

### General Procedure for the Preparation of Compound 5p

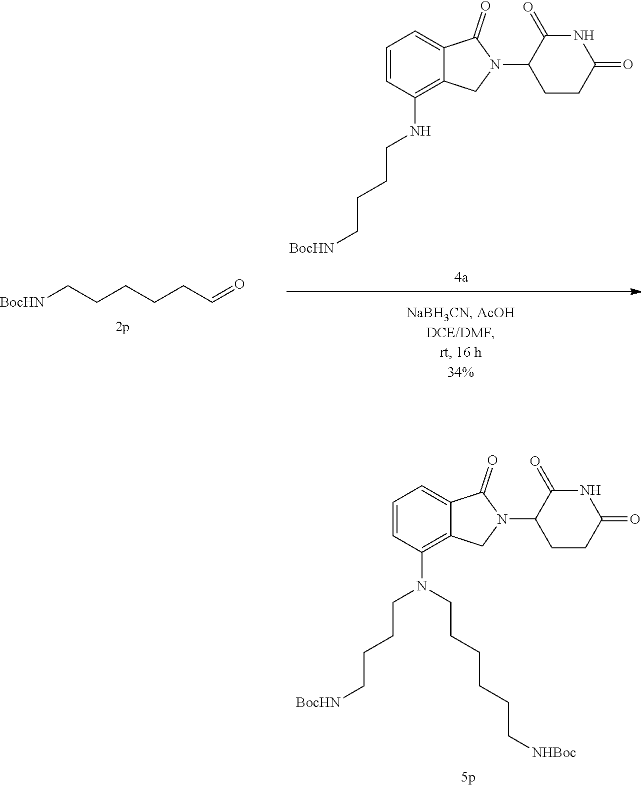 [SMILES: CC(C)(C)OC(=O)NCCCCNc1cccc2c1CN(C1CCC(=O)NC1=O)C2=O ; CC(C)(C)OC(=O)NCCCCCC=O ; CC(C)(C)OC(=O)NCCCCCCN(CCCCNC(=O)OC(C)(C)C)c1cccc2c1CN(C1CCC(=O)NC1=O)C2=O]

To a solution of compound 4a (2.07 g, 4.81 mmol, 1 eq.) and compound 2p (4.1 g, 19.32 mmol, 4.0 eq.) in DCE/DMF (20 ml) was added acetic acid (2.1 ml), molecular sieve (4.1 g) and stirred for 1 h at room temperature under nitrogen atmosphere. NaBH₃CN (1.51 g, 24.07 mmol, 5 eq.) was added in portions and stirred for 16 h at room temperature under nitrogen atmosphere. Upon completion of the reaction (as confirmed by TLC analysis, 100% EA and LCMS), the reaction was diluted with DCM (20 mL). The mixture was washed with water (3×20 mL) and brine (20 mL), dried over Na₂SO₄. The resulting mixture was concentrated under reduced pressure and purified by silica gel column chromatography (DCM/EA: 2/1) and P-HPLC (acetonitrile/H₂O: 40%-60%) to afford compound 5p (1.0 g, 34%) as a white solid.

TLC: EA=100%

R_{f} (Compound 4a)=0.5

R_{f} (Compound 5p)=0.6

LC-MS: 630.60 [M+1]⁺

### General Procedure for the Preparation of Compound 60

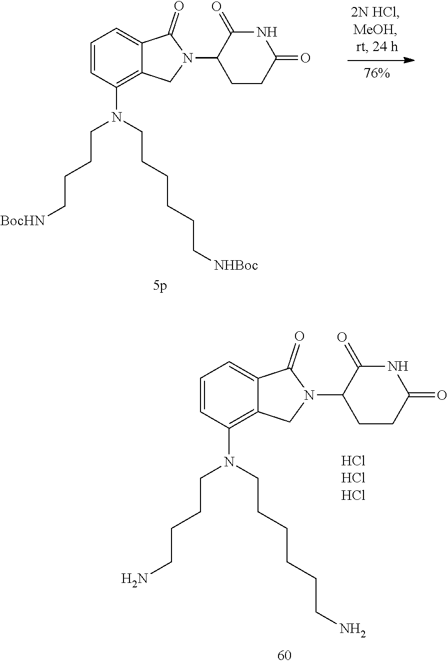 [SMILES: CC(C)(C)OC(=O)NCCCCCCN(CCCCNC(=O)OC(C)(C)C)c1cccc2c1CN(C1CCC(=O)NC1=O)C2=O ; NCCCCCCN(CCCCN)c1cccc2c1CN(C1CCC(=O)NC1=O)C2=O]

To a solution of compound 5p (200 mg, 0.318 mmol, 1 eq.) in methanol (1 ml) was added HCl (1.6 mL, 2N, 3.2 mmol, 10 eq.). The resulting solution was stirred at room temperature for 24 h. The reaction was lyophilized to afford compound 60 (130 mg, 76%) as a white solid.

TLC: DCM/methanol=10/1

R_{f} (Compound 5p)=0.6

R_{f} (Compound 60)=0.1

LC-MS: 430.40 [M+1]⁺

¹H NMR (400 MHz, D₂O): δ 7.89 (d, J=7.1 Hz, 1H), 7.80-7.67 (m, 2H), 5.13-5.01 (m, 1H), 4.53 (t, J=13.3 Hz, 2H), 3.56 (s, 4H), 2.72 (d, J=6.9 Hz, 6H), 2.40 (d, J=7.9 Hz, 1H), 2.16 (s, 1H), 1.38 (t, J=36.0 Hz, 8H), 1.12 (s, 4H).

### Compound 61

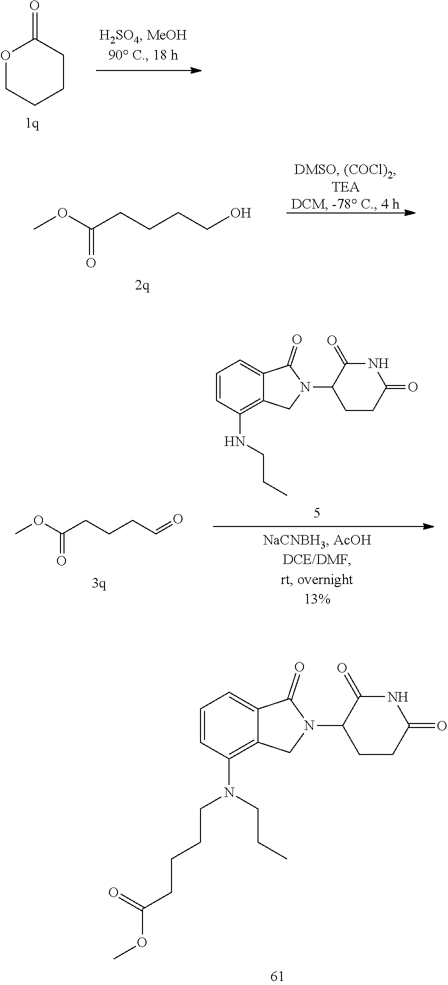

### General Procedure for the Preparation of Compound 2

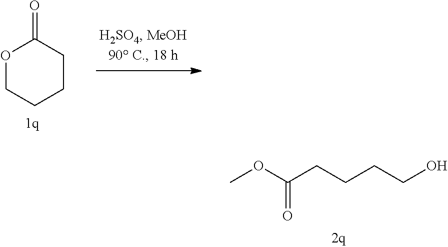

To a solution of 1q (5.0 g, 50 mmol, 1.0 eq.) in MeOH (100 mL) was added concentrated sulfuric acid (0.58 mL) and stirred for 18 h at the 90° C. The solution was cooled to 0° C., treated with NaHCO₃ (464 mg), and stirred for 10 mins. The reaction mixture was filtered and the solvent was removed in vacuum. The residue was diluted with DCM (50 mL), dried over Na₂SO₄, filtered and concentrated to afford Compound 2q (5.9 g) as colorless oil which was used without further purification.

TLC: PE/EA=4:1, KMnO₄, Ninhydrin

R_{f} (compound 1q)=0.4

R_{f} (compound 2q)=0.2

LC-MS: No found.

### General Procedure for the Preparation of Compound 3q

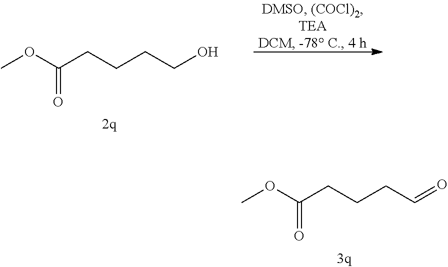

To a solution of Oxalyl chloride (7.6 g, 59.9 mmol, 1.2 eq.) in DCM (33 mL) was added dropwise to a solution of DMSO (6.6 mL) in DCM (66 mL) at −78° C. over a period of 15 minutes under nitrogen atmosphere and stirred for 15 minutes at the same temperature. The solution of Compound 2q (6.6 g, 49.9 mmol, 1.0 eq.) in DCM (33 mL) was added dropwise over a period of 15 minutes. After stirring the reaction mixture for 30 minutes at −78° C., was added TEA (15.2 g, 149.8 mmol, 3.0 eq.) in drops at −78° C. Once addition was complete, the reaction mixture was slowly warmed to 0° C. and stirred for 30 minutes. Upon completion of the reaction (as confirmed by TLC analysis, PE/EA=4/1, Rf˜0.4, KMnO₄ or Ninhydrin), the reaction mixture was diluted with DCM (10 mL). The mixture was washed with 10% citric acid (10 mL), water (10 mL) and brine (10 mL), dried over Na₂SO₄, filtered and concentrated under vacuum below 30° C. to afford Compound 3q (7.45 g, crude) as yellow oil which was used without further purification.

TLC: PE/EA=4:1, KMnO₄, Ninhydrin

R_{f} (compound 2q)=0.2

R_{f} (compound 3q)=0.4

LC-MS: No found.

¹H NMR (400 MHz, CDCl₃): δ 3.65-3.52 (m, 5H), 2.29 (td, J=7.2, 3.8 Hz, 2H), 1.88-1.75 (m, 2H), 1.53 (ddt, J=13.4, 7.4, 3.4 Hz, 2H).

### General Procedure for the Preparation of Compound 61

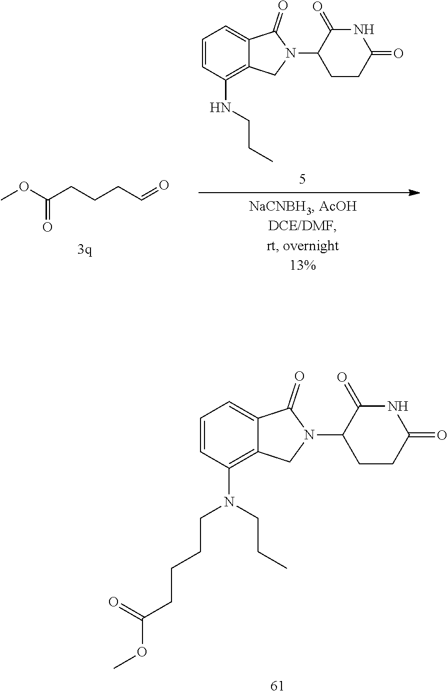

To a solution of compound 3q (2.5 g, 19.4 mmol, 1.8 eq.) and compound 5 (3.2 g, 10.8 mmol, 1.0 eq.) in DCE/DMF (60 ml, 1/1) was added acetic acid (15 ml) and stirred for 1 h at room temperature under nitrogen atmosphere. NaBH₃CN (2.7 g, 43 mmol, 4.0 eq.) was added in portions and stirred for overnight at room temperature under nitrogen atmosphere. Upon completion of the reaction (as confirmed by TLC analysis, 100% EA and LCMS), the reaction was diluted with DCM (400 mL). The mixture was washed with water (2×40 mL) and brine (2×40 mL), dried over Na₂SO₄. The resulting mixture was concentrated under reduced pressure and purified by silica gel column chromatography (DCM/EA: 2/1) and by P-HPLC (acetonitrile/H₂O: 40%-60%) to afford compound 61 (0.4 g, 13%) as a white solid.

TLC: EA=100%

R_{f} (Compound 5)=0.5

R_{f} (Compound 61)=0.6

LC-MS: 416.35 [M+1]⁺

¹H NMR (400 MHz, CD₃OD): δ 7.36 (dd, J=25.2, 7.3 Hz, 2H), 7.17 (d, J=7.8 Hz, 1H), 5.19-5.08 (m, 1H), 4.47 (s, 2H), 3.60 (s, 3H), 3.23-3.11 (m, 4H), 2.83 (dd, J=43.9, 15.0 Hz, 2H), 2.57 (d, J=13.6 Hz, 1H), 2.31 (t, J=6.6 Hz, 2H), 2.18 (d, J=12.4 Hz, 1H), 1.55 (dd, J=34.0, 6.5 Hz, 6H), 0.88 (t, J=6.9 Hz, 3H).

### Compound 63

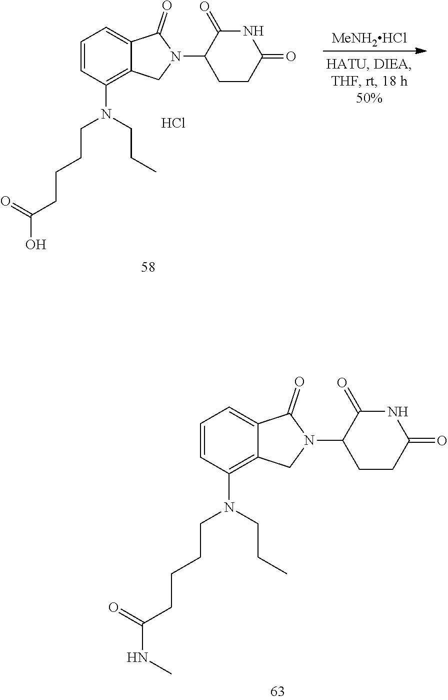 [SMILES: "MeNH2·HCl" ; CCCN(CCCCC(=O)O)c1cccc2c1CN(C1CCC(=O)NC1=O)C2=O ; Cl ; CCCN(CCCCC(=O)NC)c1cccc2c1CN(C1CCC(=O)NC1=O)C2=O]

To a solution of compound 58 (120 mg, 0.3 mmol, 1.0 eq.) in THF (2 ml) was added HATU (136 mg, 0.4 mmol, 1.2 eq.), DIEA (151 mg, 1.5 mmol, 5.0 eq.). The resulting solution was stirred at room temperature for 18 h. The resulting mixture was concentrated under reduced pressure and purified by P-HPLC (acetonitrile/H₂O: 30%-40%) to afford compound 63 (63.4 mg, 50%) as a white solid.

LC-MS: 415.35 [M+1]⁺, 413.25 [M−1]⁻

¹H NMR (400 MHz, CD₃OD): δ 7.82 (s, 1H), 7.36 (dd, J=25.5, 6.7 Hz, 2H), 7.18 (d, J=7.4 Hz, 1H), 5.13 (d, J=12.6 Hz, 1H), 4.46 (s, 2H), 3.18 (d, J=23.8 Hz, 5H), 2.83 (dd, J=40.3, 16.7 Hz, 2H), 2.65 (s, 3H), 2.56 (d, J=13.2 Hz, 1H), 2.14 (s, 3H), 1.55 (d, J=37.7 Hz, 6H), 1.32 (d, J=26.3 Hz, 3H), 0.89 (d, J=6.5 Hz, 3H).

### Hydrochloride Salt Form of Compound 66

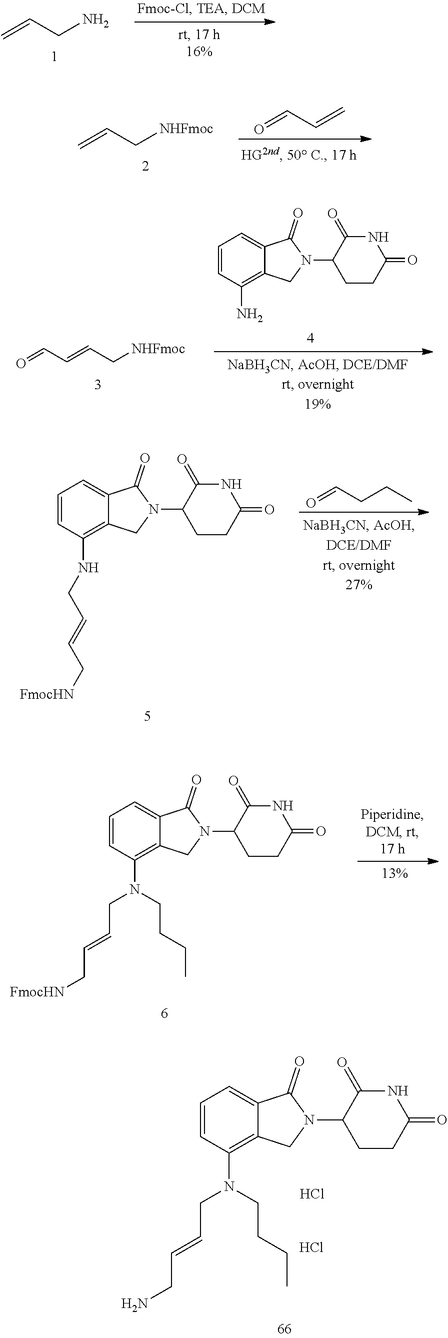

### General Procedure for the Preparation of Compound 2

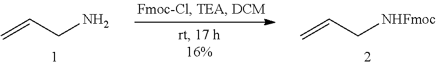

To a solution of compound 1 (0.5 g, 8.77 mmol, 1.0 eq.) in DCM (10 mL) were added TEA (2.7 g, 26.3 mmol, 3.0 eq.) and 9-Fluorenylmethyl chloroformate (3.4 g, 13.2 mmol, 1.0 eq.). The mixture was stirred for 16 h at room temperature under nitrogen atmosphere. Upon completion of the reaction (as confirmed by TLC analysis), the reaction mixture was diluted with DCM (20 mL). The mixture was washed with water (3×20 mL) and brine (20 mL), dried over Na₂SO₄. The resulting mixture was concentrated under reduced pressure and purified by silica gel column chromatography (PE/EA: 10/1) to afford compound 2 (0.4 g, 16%) as white solid.

TLC: PE/EA=1/1, Ninhydrin

R_{f} (Compound 1)=0.1

R_{f} (Compound 2)=0.8

¹H NMR (400 MHz, CDCl₃): δ 7.76 (d, J=6.4 Hz, 2H), 7.59 (d, J=6.9 Hz, 2H), 7.45-7.39 (m, 2H), 7.39-7.27 (m, 2H), 5.84 (brs, 1H), 5.25-5.05 (m, 2H), 4.82 (s, 1H), 4.45-4.30 (m, 2H), 4.22 (s, 1H), 3.82 (s, 2H).

### General Procedure for the Preparation of Compound 3

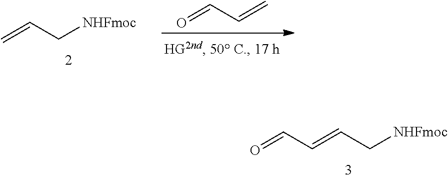

To a solution of compound 2 (0.3 g, 1.1 mmol, 1.0 eq.) in DCM (10 mL) were added acrolein (69 mg, 1.2 mmol, 1.1 eq.) and HG^{2nd} (34 mg, 0.05 mmol, 0.05 eq.). The mixture was stirred at 50° C. overnight under nitrogen atmosphere. Upon completion of the reaction (as confirmed by TLC analysis), the reaction mixture was diluted with DCM (10 mL). The mixture was washed with water (10 mL), brine (10 mL) and dried over Na₂SO₄. The resulting mixture was concentrated under reduced pressure and purified by silica gel column chromatography (PE/EA: 4/1) to afford compound 3 (0.28 g, 85%) as yellow solid.

TLC: PE/EA=3/1

R_{f} (Compound 2)=0.4

R_{f} (Compound 3)=0.2

¹H NMR (400 MHz, CDCl₃): δ 9.56 (d, J=6.1 Hz, 1H), 7.75 (s, 2H), 7.57 (s, 2H), 7.47-7.25 (m, 4H), 6.77 (d, J=14.8 Hz, 1H), 6.25-6.10 (m, 1H), 5.03 (s, 1H), 4.47 (s, 2H), 4.21 (s, 1H), 4.07 (s, 2H).

### General Procedure for the Preparation of Compound 5

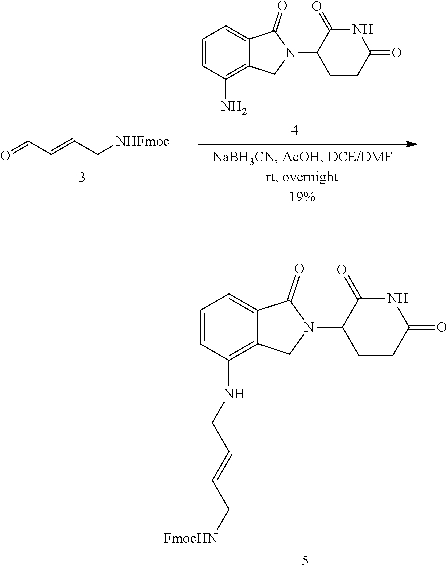

To a solution of compound 4 (450 mg, 1.7 mmol, 1.0 eq.) and compound 3 (1.1 g, 3.5 mmol, 2.0 eq.) in DCE/DMF (3/1, 8 mL) were added acetic acid (1.2 mL), molecular sieve (1.0 g). The mixture was stirred for 1 h at room temperature under nitrogen atmosphere, then NaBH₃CN (437 mg, 6.9 mmol, 4.0 eq.) was added in portions. The reaction mixture was stirred at room temperature overnight under nitrogen atmosphere. Upon completion of the reaction (as confirmed by TLC analysis and LCMS), the reaction mixture was diluted with DCM (20 mL). The mixture was washed with water (3×20 mL), brine (20 mL) and dried over Na₂SO₄. The resulting mixture was concentrated under reduced pressure and purified by silica gel column chromatography (DCM/EA: 2/1) to afford compound 5 (157 mg, 19%) as white solid.

TLC: EA=100%

R_{f} (Compound 4)=0.4

R_{f} (Compound 5)=0.5

LCMS: Calcd. for [C₃₂H₃₀N₄O₅]⁺:551, Found: 551.

### General Procedure for the Preparation of Compound 6

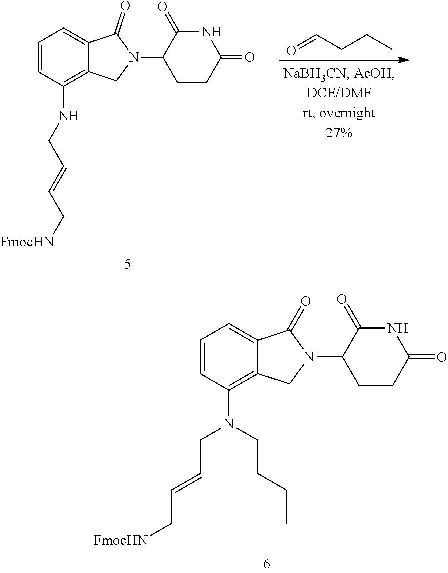

To a solution of compound 5 (137 mg, 0.25 mmol, 1.0 eq.) in DCE/DMF (3/1, 8 ml) were added butyraldehyde (71.8 mg, 1.0 mmol, 4.0 eq.), acetic acid (1.0 mL) and molecular sieve (1.0 g). The mixture was stirred for 1 h at room temperature under nitrogen atmosphere, the NaBH₃CN (62.6 mg, 1.0 mmol, 4.0 eq.) was added in portions. The reaction was stirred at room temperature overnight under nitrogen atmosphere. Upon completion of the reaction (as confirmed by TLC analysis and LCMS), the reaction mixture was diluted with DCM (20 mL). The mixture was washed with water (3×20 mL), brine (20 mL) and dried over Na₂SO₄. The resulting mixture was concentrated under reduced pressure and purified by silica gel column chromatography (PE/EA: 1/1) to afford compound 6 (41 mg, 27%) as white solid.

TLC: EA=100%

R_{f} (Compound 5)=0.5

R_{f} (Compound 6)=0.6

LCMS: Calcd. for [C₃₆H₃₈N₄O₅]+:607, Found: 607.

### General Procedure for the Preparation of Compound 66

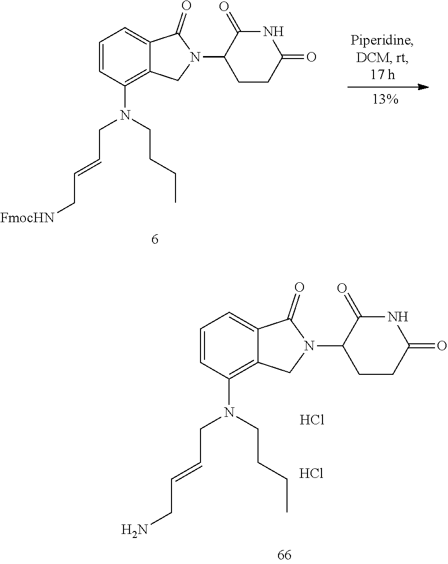

To a solution of compound 6 (47 mg, 0.1 mmol, 1.0 eq.) in DCM (4.0 mL) were added piperidine (66.7 mg, 0.8 mmol, 8.0 eq.). The mixture was stirred at room temperature overnight under nitrogen atmosphere. The reaction mixture was concentrated under reduced pressure and purified by P-HPLC (acetonitrile/H₂O: 20%-40%) to afford compound 66 (4.4 mg, 13%) as white solid.

TLC: DCM/methanol=10/1

R_{f} (Compound 6)=0.8

R_{f} (Compound 66)=0.1

LCMS: Calcd. for [C₂₁H₂₈N₄O₃]⁺:385, Found: 385.

¹H NMR (400 MHz, D₂O): δ 7.79 (d, J=6.3 Hz, 1H), 7.75-7.55 (m, 2H), 5.80-5.70 (m, 1H), 5.70-5.55 (m, 1H), 5.07 (d, J=12.5 Hz, 1H), 4.55-4.42 (m, 2H), 4.20-4.05 (m, 2H), 3.61-3.37 (m, 2H), 3.34 (s, 2H), 2.87-2.68 (m, 1H), 2.50-2.05 (m, 1H), 1.37-1.24 (m, 2H), 1.24-1.05 (m, 2H), 0.67 (s, 3H).

### Hydrochloride Salt Form of Compound 67

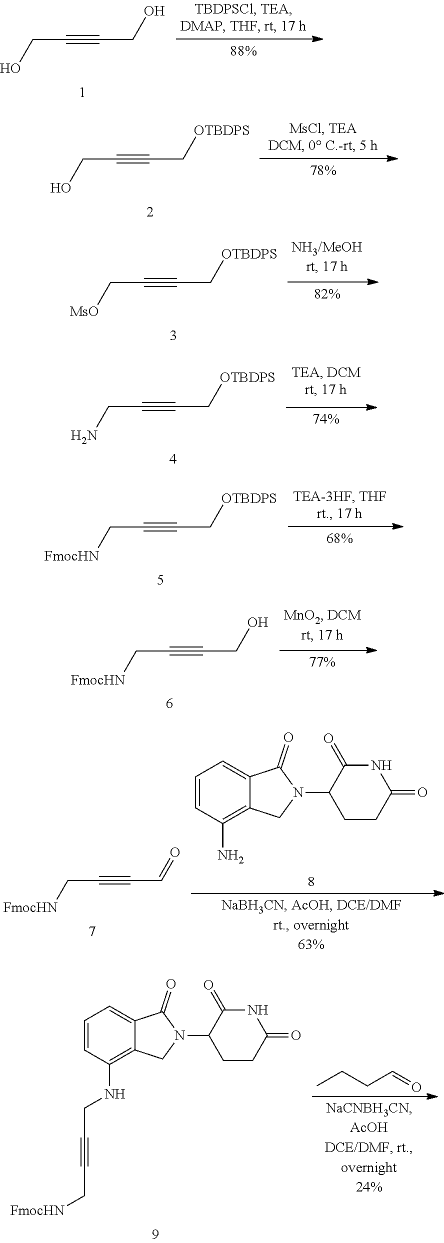

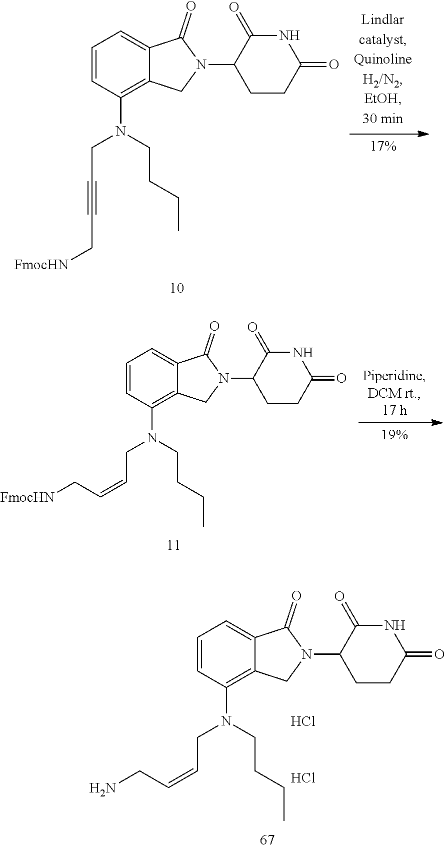

### General Procedure for the Preparation of Compound 2

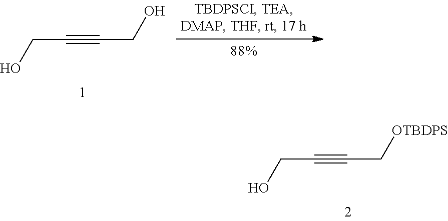

To a solution of compound 1 (3.0 g, 34.8 mmol, 1.0 eq.) and tert-butylchlorodiphenylsilane (2.9 g, 10.5 mmol, 0.3 eq.) in THF (60 mL) were added TEA (17.6 g, 174.3 mmol, 5.0 eq.), DMAP (0.4 g, 3.5 mmol, 0.1 eq.). The mixture was stirred for 17 hours at room temperature under nitrogen atmosphere. Upon completion of the reaction (as confirmed by TLC analysis), the reaction mixture was diluted with DCM (50 mL). The mixture was washed with water (3×20 mL), brine (20 mL) and dried over Na₂SO₄. The resulting mixture was concentrated under reduced pressure and purified by silica gel column chromatography (PE/EA: 10/1) to afford compound 2 (3.0 g, 88%) as clear oil.

TLC: PE/EA=5/1

R_{f} (Compound 1)=0.1

R_{f} (Compound 2)=0.3

¹H NMR (400 MHz, CDCl₃): δ 7.75-7.65 (m, 4H), 7.50-7.77 (m, 6H), 4.35 (s, 2H), 4.19 (s, 2H), 1.05 (s, 9H).

### General Procedure for the Preparation of Compound 3

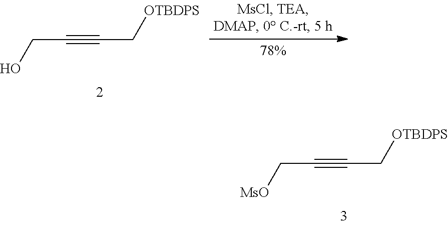

To a solution of compound 2 (3.0 g, 9.3 mmol, 1.0 eq.) in DCM (30 mL) were added TEA (2.8 g, 27.8 mmol, 3.0 eq.) and a solution of MsCl (1.59 g, 13.9 mmol, 1.5 eq.) in DCM (35 mL) at 0° C. over a period of 15 minutes under nitrogen atmosphere. The mixture was stirred for 15 minutes at the same temperature. Then the reaction mixture was slowly warmed to room temperature and stirred for 4 hours. Upon completion of the reaction (as confirmed by TLC analysis), the reaction mixture was diluted with DCM (50 mL) and washed with water (50 mL), brine (50 mL). The organic layer was dried over Na₂SO₄ and concentrated under reduced pressure. The residue was purified by silica gel column chromatography (PE/EA: 10/1) to afford compound 3 (2.9 g, 78%) as clear oil.

TLC: PE/EA=2/1

R_{f} (Compound 2)=0.60

R_{f} (Compound 3)=0.63

LCMS: Calcd. for [C₂₁H₂₆O₄SSi]⁺Na: 425, Found: 425.

¹H NMR (400 MHz, CDCl₃): δ 7.75-7.65 (m, 4H), 7.50-7.77 (m, 6H), 4.81 (s, 2H), 4.37 (s, 2H), 3.02 (s, 3H), 1.04 (s, 9H).

### General Procedure for the Preparation of Compound 4

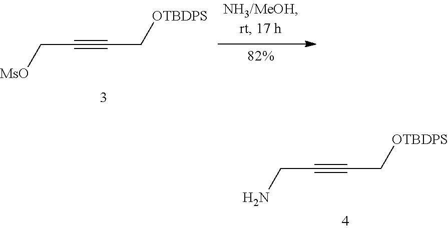

The compound 3 (2.9 g, 7.2 mmol, 1 eq.) was dissolved in NH₃/MeOH (29 mL). The mixture was stirred at room temperature for 17 hours. Upon completion of the reaction (as confirmed by TLC analysis and LCMS), the reaction mixture was concentrated under reduced pressure and purified by silica gel column chromatography (DCM/MeOH: 20/1) to afford compound 5 (1.9 g, 82%) as white solid.

TLC: DCM/EA=1/1

R_{f} (Compound 3)=0.9

R_{f} (Compound 4)=0.1

LCMS: Calcd. for [C₂₀H₂₅NOSi]⁺: 324, Found: 324.

¹H NMR (400 MHz, CDCl₃): δ 8.26 (s, 1H), 7.75-7.65 (m, 4H), 7.50-7.77 (m, 6H), 4.29 (s, 2H), 3.52 (s, 2H), 1.03 (s, 9H).

### General Procedure for the Preparation of Compound 5

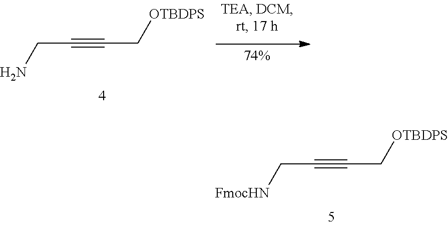

To a solution of compound 4 (2.08 g, 6.4 mmol, 1 eq.) in DCM (20 mL) were added Fmoc-Osu (2.6 g, 7.7 mmol, 1.2 eq.) and TEA (1.94 g, 19.3 mmol, 3.0 eq.). The mixture was stirred for 17 hours at room temperature under nitrogen atmosphere. Upon completion of the reaction (as confirmed by TLC analysis and LCMS), the reaction mixture was diluted with DCM (20 mL) and washed with water (3×20 mL), brine (20 mL). The organic layer was dried over Na₂SO₄ and concentrated under reduced pressure. The residue was purified by silica gel column chromatography (PE/EA: 1/1) to afford compound 5 (2.6 g, 74%) as white solid.

TLC: DCM/EA=1/1

R_{f} (Compound 4)=0.1

R_{f} (Compound 5)=0.8

LCMS: Calcd. for [C₃₅H₃₅NO₃Si]+Na: 568, Found: 568.

### General Procedure for the Preparation of Compound 6

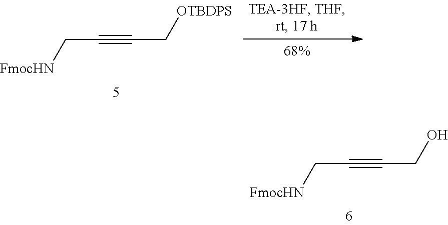

To a solution of compound 5 (2.6 g, 4.8 mmol, 1.0 eq.) in THF (1.0 ml) was added TEA-3HF (7.7 g, 47.7 mmol, 10.0 eq.). The resulting solution was stirred at room temperature overnight. Upon completion of the reaction (as confirmed by TLC analysis and LCMS), the reaction was quenched with H₂O (20 mL). The reaction mixture was extracted with EA (20 mL×3). The organic layer was washed with water (3×20 mL), brine (20 mL) and dried over Na₂SO₄. The resulting mixture was concentrated under reduced pressure and purified by silica gel column chromatography (PE/EA: 3/1) to afford compound 6 (1.0 g, 68%) as white solid.

TLC: PE/EA=2/1

R_{f} (Compound 5)=0.8

R_{f} (Compound 6)=0.2

¹H NMR (400 MHz, CDCl₃): δ 7.76 (d, J=7.5 Hz, 2H), 7.58 (d, J=7.0 Hz, 2H), 7.40 (t, J=7.4 Hz, 2H), 7.31 (t, J=7.4 Hz, 2H), 4.43 (d, J=6.8 Hz, 2H), 4.27 (s, 2H), 4.22 (s, 1H), 4.03 (s, 2H).

### General Procedure for the Preparation of Compound 7

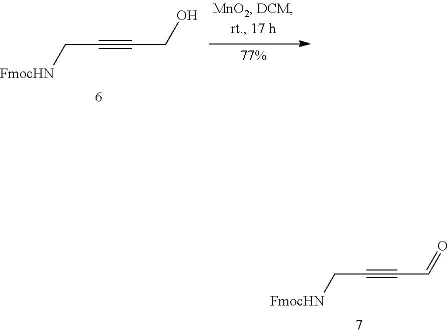

To a solution of compound 6 (1.5 g, 4.9 mmol, 1.0 eq.) in DCM (20 mL) were added MnO2 (4.24 g, 49.0 mmol, 10.0 eq.). The mixture was stirred at room temperature overnight. The reaction mixture was filtered and concentrated to afford compound 7 (1.15 m, 77%) as white solid. It was directly used in next step without further purification.

TLC: PE/EA=2/1

R_{f} (Compound 6)=0.2

R_{f} (Compound 7)=0.5

¹H NMR (399 MHz, CDCl₃): δ 9.18 (s, 1H), 7.75 (d, J=7.5 Hz, 2H), 7.57 (d, J=7.4 Hz, 2H), 7.39 (t, J=7.4 Hz, 2H), 7.30 (t, J=7.4 Hz, 2H), 5.02 (s, 1H), 4.45 (d, J=6.6 Hz, 2H), 4.27-4.10 (m, 2H).

### General Procedure for the Preparation of Compound 9

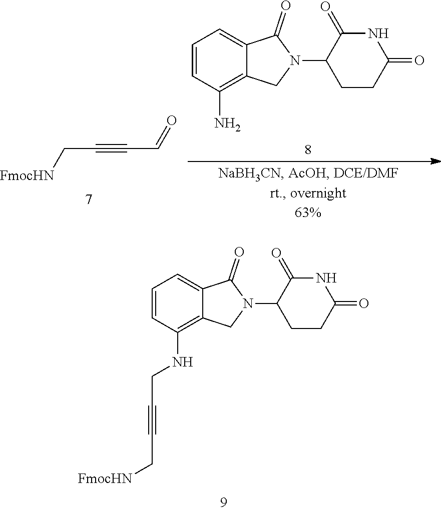

To a solution of compound 8 (1.0 g, 3.8 mmol, 1.0 eq.) and compound 7 (1.2 g, 3.8 mmol, 1.0 eq.) in DCE/DMF (6 mL) were added acetic acid (1.5 mL) and molecular sieve (1.76 g). The mixture was stirred for 1 hour at room temperature under nitrogen atmosphere. Then NaBH₃CN (1.0 g, 15.3 mmol, 4.0 eq.) was added in portions and stirred for another 16 hours at room temperature under nitrogen atmosphere. Upon completion of the reaction (as confirmed by TLC analysis and LCMS), the reaction was diluted with DCM (20 mL). The mixture was washed with water (3×20 mL), brine (20 mL) and dried over Na₂SO₄. The resulting mixture was concentrated under reduced pressure and purified by P-HPLC (acetonitrile/H₂O: 40%-60%) to afford compound 9 (1.3 g, 63%) as white solid.

TLC: EA=100%

R_{f} (Compound 8)=0.4

R_{f} (Compound 9)=0.5

### General Procedure for the Preparation of Compound 10

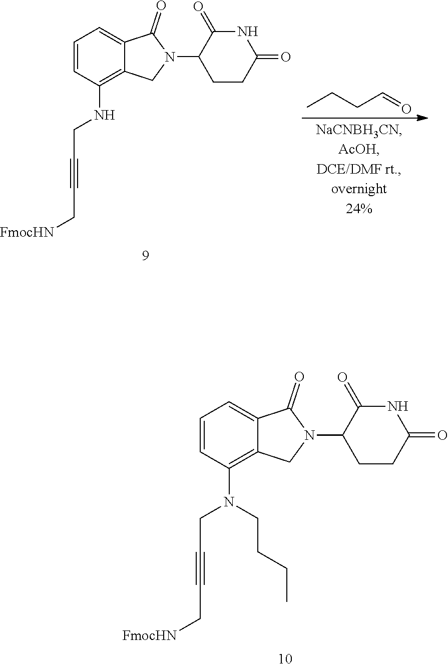

To a solution of compound 9 (1.2 g, 2.2 mmol, 1.0 eq.) and butyraldehyde (0.6 g, 8.7 mmol, 4.0 eq.) in DCE/DMF (3/1, 4 mL) were added acetic acid (1.5 ml) and molecular sieve (1.8 g). The mixture was stirred for 1 hour at room temperature under nitrogen atmosphere. Then NaBH₃CN (546 mg, 8.7 mmol, 4.0 eq.) was added in portions and stirred for another 16 hours at room temperature under nitrogen atmosphere. Upon completion of the reaction (as confirmed by TLC analysis and LCMS), the reaction mixture was diluted with DCM (20 mL). The mixture was washed with water (3×20 mL), brine (20 mL) and dried over Na₂SO₄. The solvent was removed under reduced pressure and the residue was purified by P-HPLC (acetonitrile/H₂O: 40%-60%) to afford compound 10 (315 mg, 24%) as white solid.

TLC: EA=100%

R_{f} (Compound 9)=0.5

R_{f} (Compound 10)=0.6

LCMS: Calcd. for [C₃₆H₃₆N₄O₅]⁺: 605, Found: 605.

### General Procedure for the Preparation of Compound 11

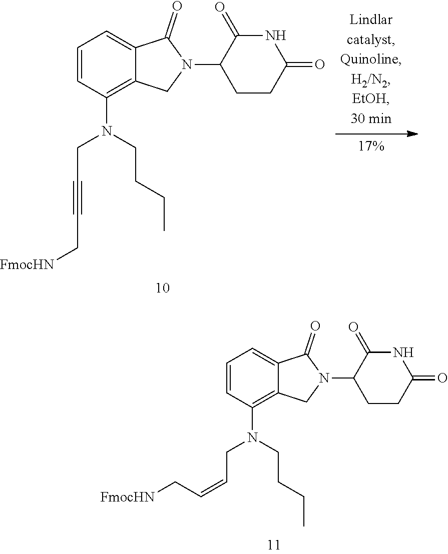

To a solution of compound 10 (587 mg, 1.0 mmol, 1.0 eq.) in EtOH (5 mL) were added Lindlar catalyst (60 mg, 10%) and quinoline (12.5 mg, 0.1 mmol, 0.1 eq.). The mixture was stirred at room temperature for 30 minutes under N₂/H₂ (50%). The reaction mixture was filtered, concentrated under reduced pressure and purified by P-HPLC (acetonitrile/H₂O: 40%-60%) to afford compound 11 (100 mg, 17%) as white solid.

TLC: DCM/EA=1/1

R_{f} (Compound 10)=0.7

R_{f} (Compound 11)=0.72

LCMS: Calcd. for [C₃₆H₃₈N₄O₅]⁺: 607, Found: 607.

### General Procedure for the Preparation of Compound 67

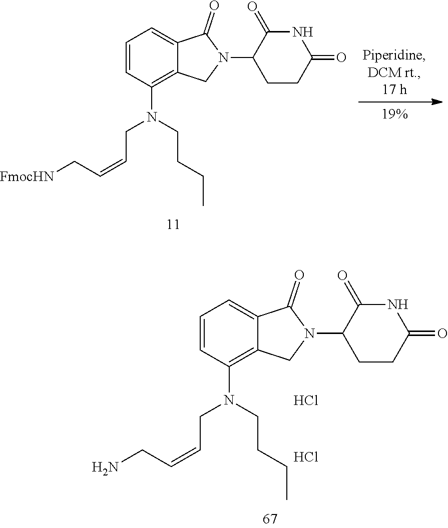

To a solution of compound 11 (100 mg, 0.17 mmol, 1.0 eq.) in DCM (5.0 mL) were added piperidine (280 mg, 3.3 mmol, 20.0 eq.). The mixture was stirred at room temperature for 17 hours. Then the reaction mixture was concentrated under reduced pressure and purified by P-HPLC (acetonitrile/H₂O: 20%-45%) to afford compound 67 (14.2 mg, 19%) as white solid.

TLC: DCM/methanol=10/1

R_{f} (Compound 11)=0.9

R_{f} (Compound 67)=0.1

LCMS: Calcd. for [C₂₁H₂₈N₄O₃]⁺: 385, Found: 385.

¹H NMR (400 MHz, D₂O): δ 7.84 (dd, J=15.1, 7.8 Hz, 2H), 7.69 (t, J=7.8 Hz, 1H), 5.72-5.62 (m, 1H), 5.62-5.50 (m, 1H), 5.05 (dd, J=13.3, 5.2 Hz, 1H), 4.51 (q, J=17.5 Hz, 2H), 4.23 (d, J=7.4 Hz, 2H), 3.66-3.52 (m, 2H), 3.38-3.21 (m, 2H), 2.85-2.65 (m, 2H), 2.39 (td, J=12.9, 7.7 Hz, 1H), 2.22-2.05 (m, 1H), 1.35-1.20 (m, 2H), 1.19-1.06 (m, 2H), 0.63 (t, J=7.3 Hz, 3H).

### Hydrochloride Salt Form of Compound 68

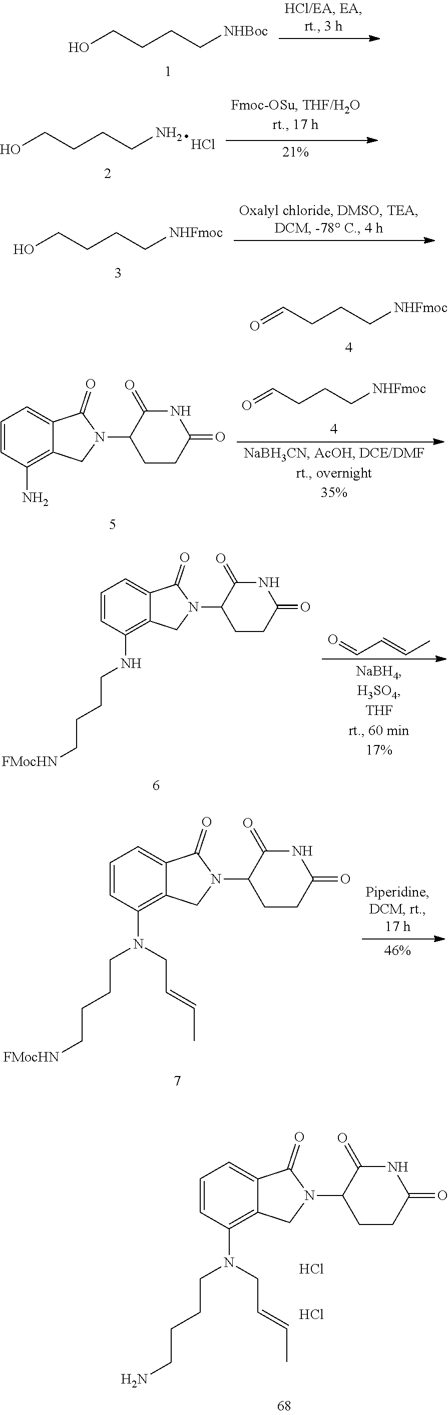

### General Procedure for the Preparation of Compound 2

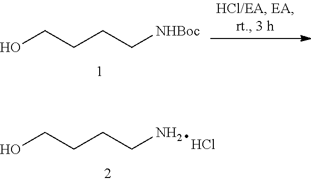 [SMILES: CC(C)(C)OC(=O)NCCCCO ; NCCCCO ; Cl]

To a solution of compound 1 (1.0 g, 5.28 mmol, 1.0 eq.) in EA (3 mL) were added HCl/EA (17.6 mL, 3 N, 52.8 mmol, 10.0 eq.) and stirred for 3 hours at room temperature under nitrogen atmosphere. Upon completion of the reaction (confirmed by TLC analysis), the solution was concentrated under vacuum to afford compound 2 (1.2 g, crude) as yellow oil which was directly used without further purification.

TLC: PE/EA=1/1 KMnO₄

R_{f} (Compound 1)=0.4

R_{f} (Compound 2)=0.1

### General Procedure for the Preparation of Compound 3

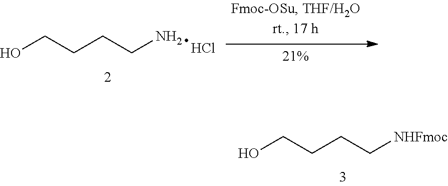

To a solution of compound 2 (0.3 g, 3.4 mmol, 1.0 eq.) in THF/H₂O (3/1, 8 mL) were added Fmoc-Osu (1.7 g, 5.1 mmol, 1.5 eq.) and TEA (340 mg, 3.4 mmol, 1.0 eq.). The mixture was stirred at room temperature overnight. Upon completion of the reaction (as confirmed by TLC analysis), the reaction mixture was diluted with DCM (10 mL). The mixture was washed with water (10 mL), brine (5 mL) and dried over Na₂SO₄. The resulting mixture was concentrated under reduced pressure and purified by silica gel column chromatography (PE/EA: 5/1) to afford compound 3 (160 mg, 21%) as white solid.

TLC: PE/EA=2/1

R_{f} (Compound 2)=0.1

R_{f} (Compound 3)=0.7

¹H NMR (400 MHz, CDCl₃): δ 7.75 (d, J=7.5 Hz, 2H), 7.57 (d, J=7.4 Hz, 2H), 7.38 (t, J=7.3 Hz, 2H), 7.30 (t, J=8.5, 2H), 4.50-4.4 (i, 2H), 4.25-4.20 (m, 1H), 3.65 (s, 2H), 3.21 (s, 2H), 1.75-1.37 (i, 4H).

### General Procedure for the Preparation of Compound 4

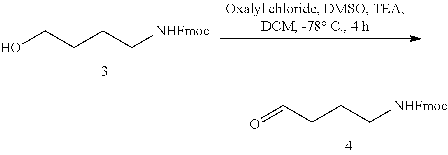

To a solution of oxalyl chloride (200 mg, 1.6 mmol, 1.2 eq.) in DCM (3 mL) were added dropwise a solution of DMSO (0.4 mL, 1.0 V) in DCM (2 mL) at −78° C. over a period of 15 minutes under nitrogen atmosphere and stirred for 15 minutes at the same temperature. The solution of Compound 3 (410 mg, 1.32 mmol, 1.0 eq.) in DCM (3 mL) was added to the previous solution over a period of 15 minutes at −78° C. After the mixture was stirred for 30 minutes at −78° C., TEA (400 mg, 3.96 mmol, 3.0 eq.) was added at −78° C. Once addition was complete, the reaction mixture was slowly warmed to 0° C. and stirred for 30 minutes. Upon completion of the reaction (as confirmed by TLC analysis), the reaction mixture was diluted with DCM (20 mL) and quenched with 10% aq. citric acid solution (10 mL). The result solution was washed with water (20 mL), brine (20 mL) and dried over Na₂SO₄. The solvent was removed under vacuum below 30° C. to afford Compound 4 (640 mg, crude) as yellow oil which was directly used without further purification.

TLC: PE/EA=4:1, KMnO₄, Ninhydrin

R_{f} (Compound 3)=0.4

R_{f} (Compound 4)=0.6

### General Procedure for the Preparation of Compound 6

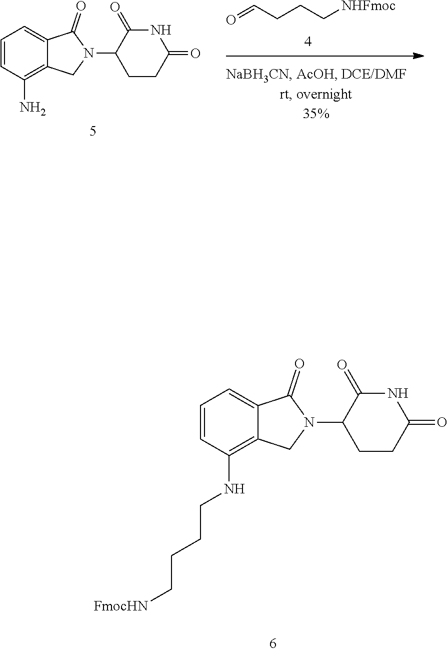

To a solution of compound 5 (410 mg, 1.6 mmol, 1.0 eq.) and compound 4 (979 mg, 3.2 mmol, 2.0 eq.) in DCE/DMF (10 mL) were added acetic acid (1.5 ml), molecular sieve (1.0 g). The mixture was stirred for 1 hour at room temperature under nitrogen atmosphere. Then NaBH₃CN (398 mg, 6.4 mmol, 4.0 eq.) was added in portions and stirred at room temperature overnight under nitrogen atmosphere. Upon completion of the reaction (as confirmed by TLC analysis), the reaction mixture was diluted with DCM (20 mL). The mixture was washed with water (3×30 mL), brine (30 mL) and dried over Na₂SO₄. The resulting mixture was concentrated under reduced pressure and purified by P-HPLC (acetonitrile/H₂O: 40%-60%) to afford compound 6 (315 mg, 35%) as white solid.

TLC: EA=100%

R_{f} (Compound 5)=0.4

R_{f} (Compound 6)=0.5

### General Procedure for the Preparation of Compound 7

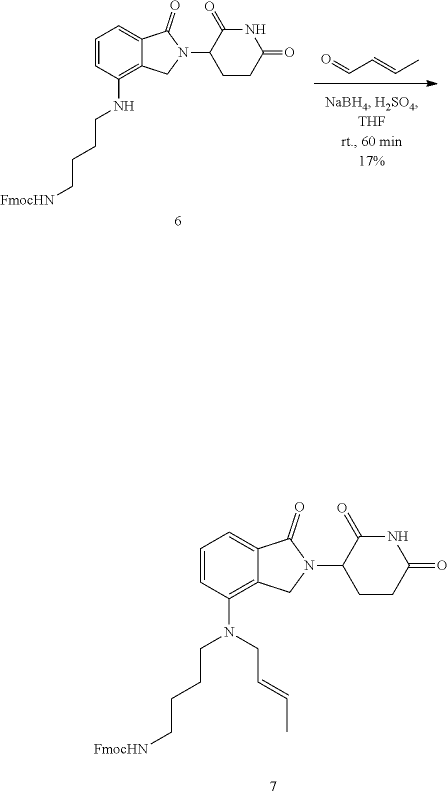

To a solution of compound 6 (642 mg, 1.2 mmol, 1.0 eq.) in THF (3.0 mL) were added dropwise a solution of crotonaldehyde (87.6 mg, 1.3 mmol, 1.1 eq.) of H₂SO₄ (3.11 mL, 4 N, 12.4 mmol, 10.7 eq.) at 0° C. Then NaBH₄ (26.8 mg, 0.7 mmol, 0.6 eq.) was added at 0° C. and stirred at same temperature for 40 minutes. The reaction mixture was poured into ice water, extracted with DCM (3×30 mL). The organic layer was washed with H₂O, brine and concentrated under reduced pressure. The residue was purified by P-HPLC (acetonitrile/H₂O: 40%-60%) to afford compound 7 (120 mg, 17%) as white solid.

TLC: EA=100%

R_{f} (Compound 6)=0.5

R_{f} (Compound 7)=0.6

LCMS: Calcd. for [C₃₆H₃₈N₄O₅]+: 607, Found: 607.

### General Procedure for the Preparation of Compound 68

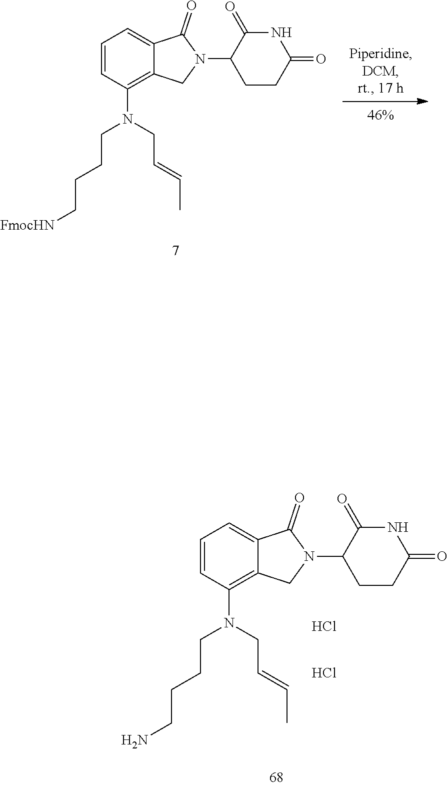

To a solution of compound 7 (120 mg, 0.2 mmol, 1.0 eq.) in DCM (4.0 mL) were added piperidine (171 mg, 2.0 mmol, 10.0 eq.). The mixture was stirred at room temperature for 17 hours. After concentrated under vacuum, the residue was purified by P-HPLC (acetonitrile/H₂O: 20%-40%) to afford compound 68 (42.5 mg, 46%) as white solid.

TLC: DCM/methanol=10/1

R_{f} (Compound 7)=0.5

R_{f} (Compound 68)=0.1

LCMS: Calcd. for [C₂₁H₂₈N₄O₃]⁺: 385, Found: 385.

¹H NMR (400 MHz, D₂O): δ 7.76 (d, J=7.4 Hz, 1H), 7.69 (d, J=8.1 Hz, 1H), 7.60 (t, J=7.8 Hz, 1H), 5.75-5.55 (m, 1H), 5.16-5.04 (m, 1H), 4.98 (dd, J=13.3, 5.1 Hz, 1H), 4.42 (dd, J=35.2, 17.5 Hz, 2H), 3.96 (d, J=7.2 Hz, 2H), 3.62-3.41 (m, 2H), 2.75-2.55 (m, 4H), 2.32 (qd, J=13.0, 5.1 Hz, 1H), 2.10-2.01 (m, 1H), 1.51-1.24 (m, 7H).

### Hydrochloride Salt Form of Compound 69

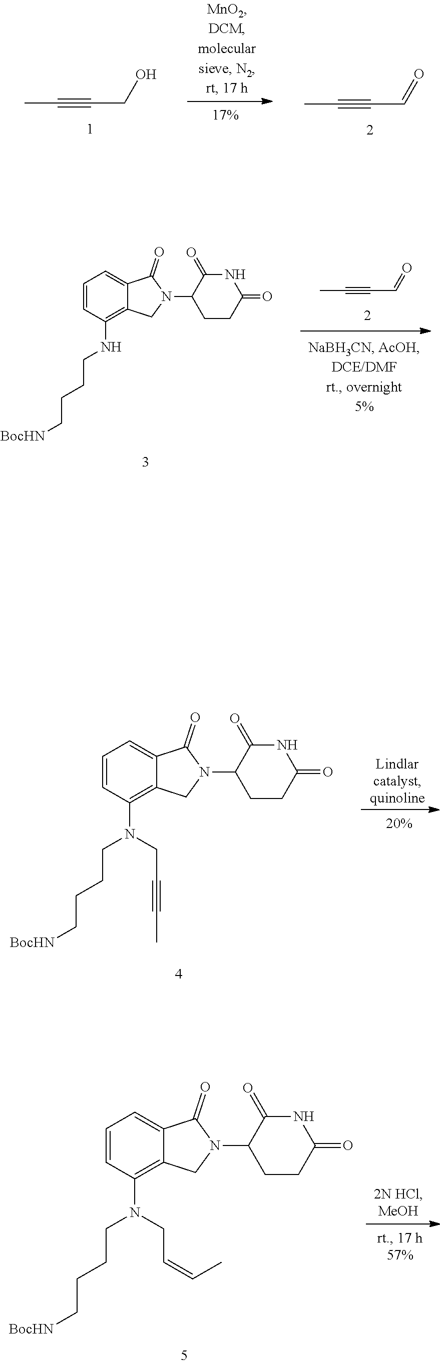

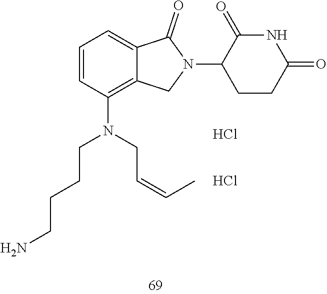 [SMILES: C/C=C\CN(CCCCN)c1cccc2c1CN(C1CCC(=O)NC1=O)C2=O ; Cl ; Cl]

### General Procedure for the Preparation of Compound 2

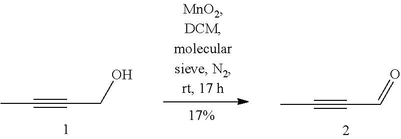

To a solution of compound 1 (620 mg, 8.8 mmol, 1.0 eq.) in DCM (30 mL) were added MnO₂ (7.7 g, 88.5 mmol, 10.0 eq.), molecular sieve (3.1 g) and stirred for 17 hours at room temperature. Upon completion of the reaction (as confirmed by TLC analysis), the reaction mixture was filtered and concentrated under reduced pressure below 30° C. to afford compound 2 (110 mg, 17%) as a clear oil which was directly used without further purification.

TLC: PE/EA=3/1 KMnO₄

R_{f} (Compound 1)=0.3

R_{f} (Compound 2)=0.3

¹H NMR (400 MHz, CDCl₃): δ 9.13 (s, 1H), 2.05 (s, 3H).

### General Procedure for the Preparation of Compound 4

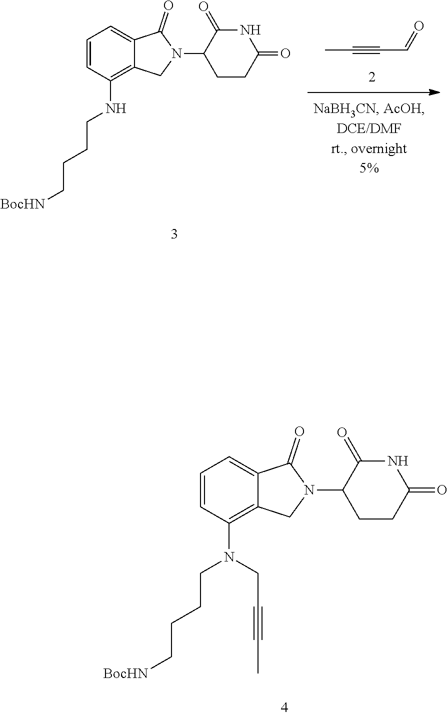

To a solution of compound 3 (2.0 g, 4.6 mmol, 1.0 eq.) and compound 2 (1.62 g, 18.4 mmol, 4.0 eq.) in DCE/DMF (3/1, 8 mL) were added acetic acid (2.0 mL), molecular sieve (4.0 g). The mixture was stirred for 1 hour at room temperature under nitrogen atmosphere. Then NaBH₃CN (1.2 g, 18.4 mmol, 4.0 eq.) was added in portions and stirred overnight at room temperature under nitrogen atmosphere. Upon completion of the reaction (as confirmed by TLC analysis and LCMS), the reaction mixture was diluted with DCM (20 mL). The mixture was washed with water (3×20 mL), brine (20 mL) and dried over Na₂SO₄. The resulting mixture was concentrated under reduced pressure and purified by P-HPLC (acetonitrile/H₂O: 40%-60%) to afford compound 4 (110 mg, 5%) as white solid.

TLC: EA=100%

R_{f} (Compound 3)=0.4

R_{f} (Compound 4)=0.5

LCMS: Calcd. for [C₂₆H₃₄N₄O₅]⁺: 483, Found: 483.

### General Procedure for the Preparation of Compound 5

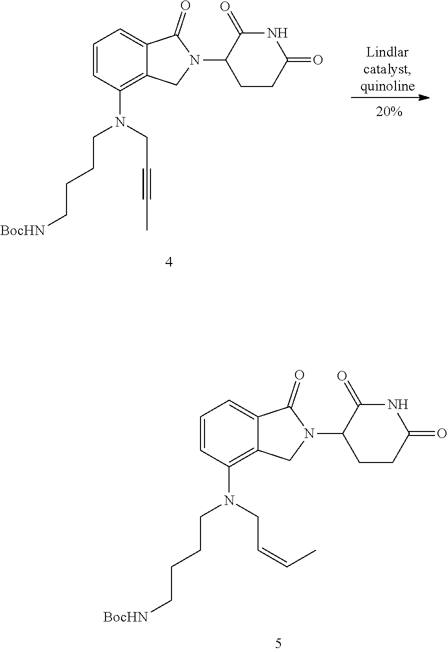 [SMILES: CC#CCN(CCCCNC(=O)OC(C)(C)C)c1cccc2c1CN(C1CCC(=O)NC1=O)C2=O ; C/C=C\CN(CCCCNC(=O)OC(C)(C)C)c1cccc2c1CN(C1CCC(=O)NC1=O)C2=O]

To a solution of compound 4 (100 mg, 0.2 mmol, 1.0 eq.) in EtOH (5 mL) were added Lindlar catalyst (10 mg, 10%) and quinoline (1.8 mg, 0.02 mmol, 0.1 eq.). The mixture was stirred at room temperature for 30 minutes, filtered and concentrated under reduced pressure. The residue was purified by P-HPLC (acetonitrile/H₂O: 40%-60%) to afford compound 5 (20 mg, 20%) as white solid.

TLC: DCM/methanol=1/1

R_{f} (Compound 4)=0.6

R_{f} (Compound 5)=0.62

LCMS: Calcd. for [C₂₆H₃₆N₄O₅]⁺: 485, Found: 485.

### General Procedure for the Preparation of Compound 69

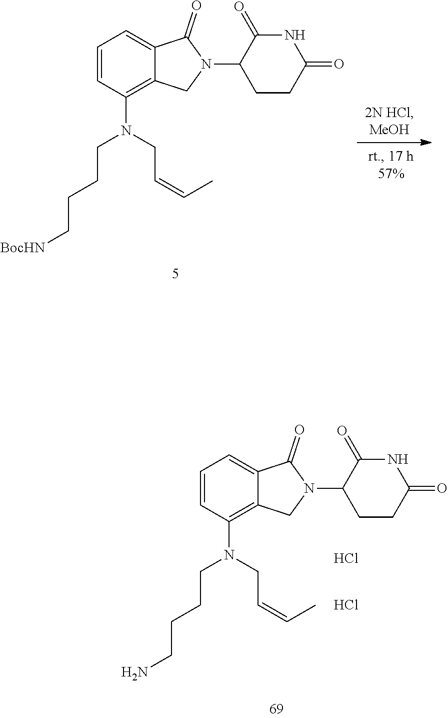 [SMILES: C/C=C\CN(CCCCNC(=O)OC(C)(C)C)c1cccc2c1CN(C1CCC(=O)NC1=O)C2=O ; C/C=C\CN(CCCCN)c1cccc2c1CN(C1CCC(=O)NC1=O)C2=O ; Cl ; Cl]

To a solution of compound 5 (33 mg, 0.07 mmol, 1.0 eq.) in MeOH (1 mL) were added HCl (0.4 mL, 2 N, 0.8 mmol, 11.0 eq.). The mixture was stirred at room temperature for 24 hours and concentrated under reduced pressure. The residue was purified by Biotage (C18, acetonitrile/H₂O: 30%-60%) to afford compound 69 (17.8 mg, 57%) as white solid.

TLC: DCM/methanol=10/1

R_{f} (Compound 5)=0.5

R_{f} (Compound 69)=0.1

LCMS: Calcd. for [C₂₁H₂₈N₄O₃]⁺: 385, Found: 385.

¹H NMR (400 MHz, D₂O): δ 7.87-7.77 (m, 2H), 7.72-7.61 (m, 1H), 5.80-5.61 (m, 1H), 5.27-5.12 (m, 1H), 5.04 (d, J=10.0 Hz, 1H), 4.50 (q, J=17.4 Hz, 2H), 4.16 (d, J=6.2 Hz, 2H), 3.70-3.55 (m, 2H), 2.82-2.61 (m, 4H), 2.48-2.30 (m, 1H), 2.20-2.04 (m, 1H), 1.58-1.31 (m, 4H), 1.22 (d, J=5.2 Hz, 3H).

### Hydrochloride Salt Form of Compound 80

### 3-(4-((4-(methylamino)butyl)(pentyl)amino)-1-oxoisoindolin-2-yl)piperidine-2,6-dione.Hydrochloride

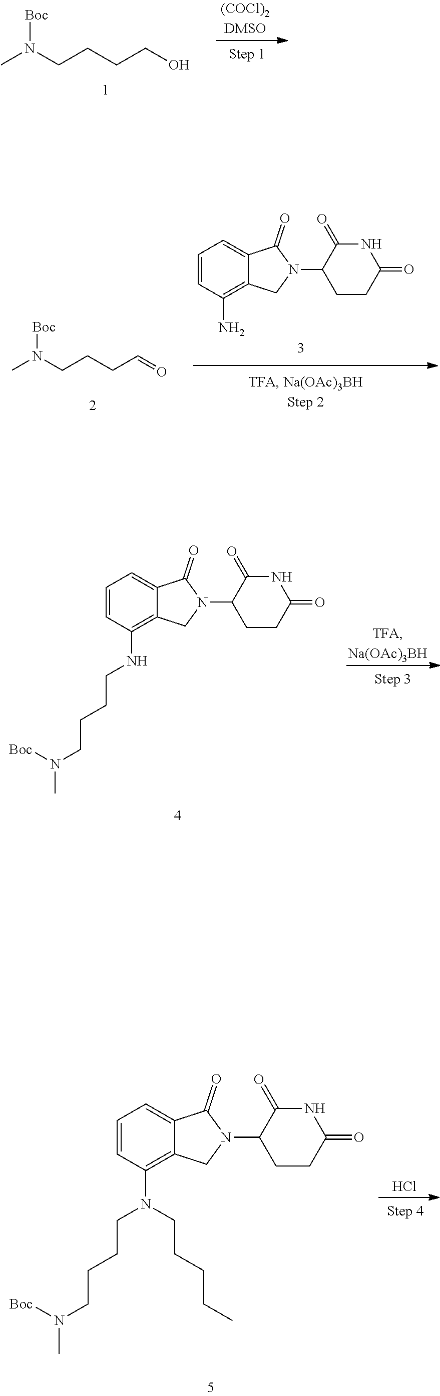 [SMILES: CN(CCCCO)C(=O)OC(C)(C)C ; CN(CCCC=O)C(=O)OC(C)(C)C ; Nc1cccc2c1CN(C1CCC(=O)NC1=O)C2=O ; CN(CCCCNc1cccc2c1CN(C1CCC(=O)NC1=O)C2=O)C(=O)OC(C)(C)C ; CCCCCN(CCCCN(C)C(=O)OC(C)(C)C)c1cccc2c1CN(C1CCC(=O)NC1=O)C2=O ; Cl]

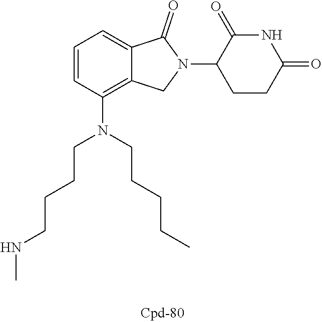 [SMILES: CCCCCN(CCCCNC)c1cccc2c1CN(C1CCC(=O)NC1=O)C2=O]

### Step-1: tert-butyl methyl(4-oxobutyl)carbamate

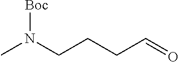

To a solution of oxalyl chloride (0.25 mL, 2.95 mmol, 1.2 equiv) in DCM (5 mL) was added DMSO (0.419 mL, 5.90 mmol, 2.4 equiv) at −78° C. in drops, over a period of 5 min under nitrogen atmosphere and stirred for 15 min at the same temperature. To this, a solution of tert-butyl (4-hydroxybutyl)(methyl)carbamate 1 (500 mg, 5.90 mmol, 1 equiv) in DCM (2.5 mL) was added dropwise over a period of 5 minutes. After stirring the reaction mixture for 30 min at −78° C., triethylamine (1.713 mL, 12.30 mmol, 5 equiv) was added in drops at −78° C. After the addition, the reaction mixture was slowly warmed to 0° C. and stirred for 30 min. Upon completion of the reaction (as confirmed by TLC analysis, 50% EtOAc/pet ether, R_{f}˜0.4, KMnO₄), the reaction mixture was diluted with DCM (20 mL) and washed with 10% aq citric acid solution (20 mL), water (1×10 mL) and brine (1×10 mL), dried over anhy. Na₂SO₄, filtered and concentrated under vacuum (below 30° C.) to afford tert-butyl methyl(4-oxobutyl)carbamate 2 (450 mg, crude) as pale yellow liquid which was used further without purification.

¹H-NMR (400 MHz, DMSO-d₆): δ 9.67 (s, 1H), 3.16 (t, J=7.20 Hz, 2H), 2.69 (s, 3H), 2.43-2.38 (m, 2H), 1.71 (t, J=6.80 Hz, 2H), 1.41 (s, 3H).

### Step-2: tert-butyl (4-((2-(2,6-dioxopiperidin-3-yl)-1-oxoisoindolin-4-yl)amino)butyl)(methyl)carbamate

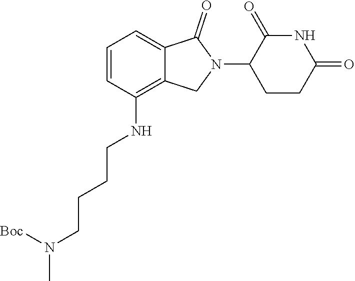 [SMILES: CN(CCCCNc1cccc2c1CN(C1CCC(=O)NC1=O)C2=O)C(=O)OC(C)(C)C]

A mixture of tert-butyl methyl(4-oxobutyl)carbamate (279 mg, 1.389 mmol, 1.2 equiv) and Lenolidamide 3 (300 mg, 1.157 mmol, 1 equiv) were dissolved in a mixture of 1,2-Dichloroethane (3 mL, 10 vol) and DMF (3 mL, 10 vol) under nitrogen atmosphere. To this solution, TFA (0.08 mL, 1.041 mmol, 0.9 equiv) and Na(OAc)₃BH (368 mg, 1.736 mmol, 1.5 equiv) were added in portions at 0° C. and stirred for 16 h at room temperature. Upon completion of the reaction (as confirmed by TLC analysis, 100% EtOAc, R_{f}˜0.4, and UPLC), the reaction mixture was diluted with DCM (20 mL), washed with water (3×20 mL), brine (20 mL), dried over Na₂SO₄, filtered and concentrated to give the crude product, which was purified by Isolera (Biotage R snap cartridge, KP-Sil, 25 g, 230-400 mesh silica gel) using 80-85% ethyl acetate in pet ether. The pure fractions were collected and concentrated under vacuum to give tert-butyl (4-((2-(2,6-dioxopiperidin-3-yl)-1-oxoisoindolin-4-yl)amino)butyl)(methyl)carbamate 4 (350 mg, 0.760 mmol, 65% yield) as off white solid.

¹H-NMR (400 MHz, DMSO-d₆): δ 11.00 (s, 1H), 7.29 (t, J=7.60 Hz, 1H), 6.93 (d, J=6.80 Hz, 1H), 6.75 (d, J=8.40 Hz, 1H), 5.61 (s, 1H), 5.12 (q, J=5.20 Hz, 1H), 4.23 (d, 1H), 4.13 (d, J=17.20 Hz, 1H), 3.21-3.14 (m, 3H), 2.88 (q, 2H), 2.72 (t, 3H), 2.58 (d, 2H), 2.46-2.28 (m, 2H), 1.55 (s, 3H), 1.37 (s, 9H).

LCMS:345.1 (M-Boc), Rt (min): 2.267, Area %: 96.498.

### Step-3: tert-butyl (4-((2-(2,6-dioxopiperidin-3-yl)-1-oxoisoindolin-4-yl)(pentyl)amino)butyl)(methyl)carbamate

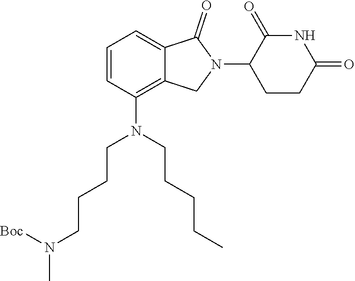 [SMILES: CCCCCN(CCCCN(C)C(=O)OC(C)(C)C)c1cccc2c1CN(C1CCC(=O)NC1=O)C2=O]

A mixture of tert-butyl (4-((2-(2,6-dioxopiperidin-3-yl)-1-oxoisoindolin-4-yl)amino)butyl)(methyl)carbamate 4 (350 mg, 0.787 mmol, 1 equiv) and pentanal (339 mg, 3.94 mmol, 5 equiv) were dissolved in a mixture of CH₂Cl₂ (3.5 mL, 10 vol) and DMF (3.5 mL, 10 vol) under nitrogen atmosphere. To this TFA (0.05 mL, 0.709 mmol, 0.9 equiv) and STAB (250 mg, 1.181 mmol, 1.5 equiv) were added in portions at 0° C. and stirred for 16 h at room temperature. Upon completion of the reaction (as confirmed by TLC analysis, 100% EtOAc, R_{f} 0.6, and UPLC), the reaction mixture was diluted with DCM (20 mL), washed water (3×20 mL), brine (20 mL), dried over anhy. Na₂SO₄, filtered and concentrated to give the crude product. The crude product was purified by Isolera (Biotage R snap cartridge, KP-Sil, 25 g, Silica gel 230-400 mesh) using 70-80% ethyl acetate in pet ether. The pure fractions were collected and concentrated under vacuum to give the desired product to afford tert-butyl(4-((2-(2,6-dioxopiperidin-3-yl)-1-oxoisoindolin-4-yl)(pentyl)amino)butyl)(methyl)carbamate 5 (130 mg, 0.241 mmol, 30% yield) as pale yellow solid.

¹H-NMR (400 MHz, DMSO-d₆): δ 10.98 (s, 1H), 7.38 (t, J=8.00 Hz, 1H), 7.19 (d, J=7.20 Hz, 1H), 7.08 (d, J=8.40 Hz, 1H), 5.11 (q, J=5.20 Hz, 1H), 4.34 (d, 1H), 4.29 (d, 1H), 3.22-3.11 (m, 6H), 2.92-2.87 (m, 2H), 2.70 (q, J=2.00 Hz, 3H), 2.57 (s, 2H), 2.01 (t, J=5.20 Hz, 1H), 1.25-1.40 (m, 18H), 0.84 (t, J=7.20 Hz, 3H).

LCMS: 515.4 (M+H), Rt (min): 1.989, Area %: 95.589.

### Step-4: 3-(4-((4-(methylamino)butyl)(pentyl)amino)-1-oxoisoindolin-2-yl)piperidine-2,6-dione

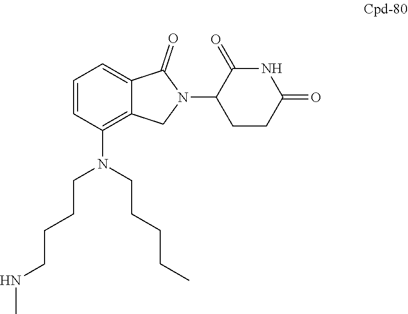 [SMILES: CCCCCN(CCCCNC)c1cccc2c1CN(C1CCC(=O)NC1=O)C2=O]

To an ice cold suspension of tert-Butyl (4-((2-(2,6-dioxopiperidin-3-yl)-1-oxoisoindolin-4-yl)amino)butyl)carbamate (130 mg, 0.240 mmol, 1 equiv) in DCM (1.5 mL) was added HCl (4M soln. in EtOAc, 0.7 mL) and stirred for 2 h at room temperature. The progress of the reaction was monitored by LCMS. The reaction mixture was concentrated and the residue was triturated with MTBE (10 ml). The solid obtained was lyophilized to get 3-(4-((4-(methylamino)butyl)(pentyl)amino)-1-oxoisoindolin-2-yl)piperidine-2,6-dione.hydrocholide 80 (70 mg, 63.2%) as pale yellow solid.

¹H-NMR (400 MHz, DMSO-d₆): δ 11.01 (s, 1H), 8.83 (s, 2H), 7.53 (s, 3H), 5.15-5.10 (m, 1H), 4.48-4.49 (m, 2H), 3.31 (s, 4H), 2.97-2.88 (m, 1H), 2.82 (s, 1H), 2.74 (s, 2H), 2.51-2.48 (m, 3H), 1.91 (s, 1H), 1.59 (t, J=6.40 Hz, 2H), 1.46 (d, J=17.20 Hz, 3H), 1.24 (s, 4H), 0.82 (t, J=6.80 Hz, 3H).

LCMS: 415.2 (M+H), Method: Mobile phase: A: 0.1% FA in H₂O, Mobile phase: B: ACN, Column: Atlantis dC18 (50×4.6 mm) 5 μm, Flow Rate: 1.5 ml/min Rt (min): 1.599, Area %: 95.709.

HPLC: Method: Mobile phase:A: 0.1% TFA in water, Mobile phase: B: ACN, Column:X-Bridge C8(50×4.6) mm, 3.5 μm Flow: 2.0 mL/min, Rt (min): 2.409, Area %: 97.597.

### Formic acid salt Form of Compound 81

### 3-(4-((4-(dimethylamino)butyl)(pentyl)amino)-1-oxoisoindolin-2-yl)piperidine-2,6-dione formic acid salt

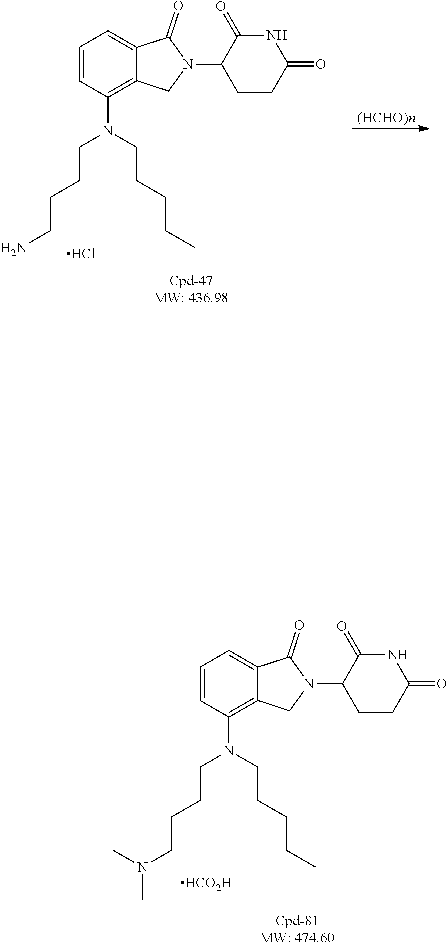 [SMILES: CCCCCN(CCCCN)c1cccc2c1CN(C1CCC(=O)NC1=O)C2=O ; "(HCHO)n" ; Cl ; CCCCCN(CCCCN(C)C)c1cccc2c1CN(C1CCC(=O)NC1=O)C2=O ; "·HCO2H"]

To a solution of 3-(4-((4-aminobutyl)(pentyl)amino)-1-oxoisoindolin-2-yl)piperidine-2,6-dione 47 (300 mg, 0.749 mmol) and paraformaldehyde (450 mg, 14.98 mmol, 20 equiv) in DCM (6 mL, 20 vol) was added AcOH (0.129 ml, 2.247 mmol, 3 equiv) and the reaction mixture was stirred for 2 h at room temperature. NaCNBH₄ (188 mg, 3.00 mmol, 4 equiv) was added to the mixture in portions and the reaction mixture was stirred for additional 40 h at room temperature. Upon completion of the reaction (as confirmed by TLC analysis, EtOAc, R_{f}˜0.4), the reaction mixture was diluted with DCM (10 mL), washed with water (2×20 mL), brine (20 mL), dried over anhy. Na₂SO₄, filtered and concentrated. The resulted residue was purified by prep.HPLC (HCOOH:ACN method) and lyophilized to get 3-(4-((4-(dimethylamino)butyl)(pentyl)amino)-1-oxoisoindolin-2-yl)piperidine-2,6-dione 81 (26 mg, 0.059 mmol, 7.90% yield) as white solid.

¹H-NMR (400 MHz, DMSO-d₆): δ 11.01 (s, 1H), 7.42-7.38 (m, 1H), 7.22 (t, J=7.20 Hz, 1H), 7.12 (q, J=3.20 Hz, 1H), 5.23-5.18 (m, 1H), 5.14-5.05 (m, 1H), 4.40 (d, J=7.20 Hz, 1H), 4.32 (d, J=4.40 Hz, 1H), 3.25-3.17 (m, 4H), 2.82-2.68 (m, 3H), 2.53-2.50 (m, 6H), 2.34 (s, 2H), 1.63 (q, J=3.20 Hz, 2H), 1.44 (d, J=6.40 Hz, 4H), 1.27-1.23 (m, 4H), 0.85-0.9 (m, 3H).

LCMS: 429.3 (M+H). Method: Mobile Phase A: 0.1% TFA in H₂O. Mobile Phase B: 0.1% TFA in ACN. Flow rate: 1.5 mL/min. Column: XBridge C8 (50×4.6 mm) 3.5 μm. Rt (min): 1.824; Area %: 97.584.

HPLC: Method: Mobile Phase A: 0.1% TFA in H₂O. Mobile Phase B: Acetonitrile. Flow rate: 2.0 mL/min. Column: X-Bridge C8(50×4.6) mm, 3.5 μm. Rt (min): 3.658; Area %: 97.694.

### Compound 82

### N-(4-((2-(2,6-dioxopiperidin-3-yl)-1-oxoisoindolin-4-yl)(pentyl)amino)butyl)acetamide

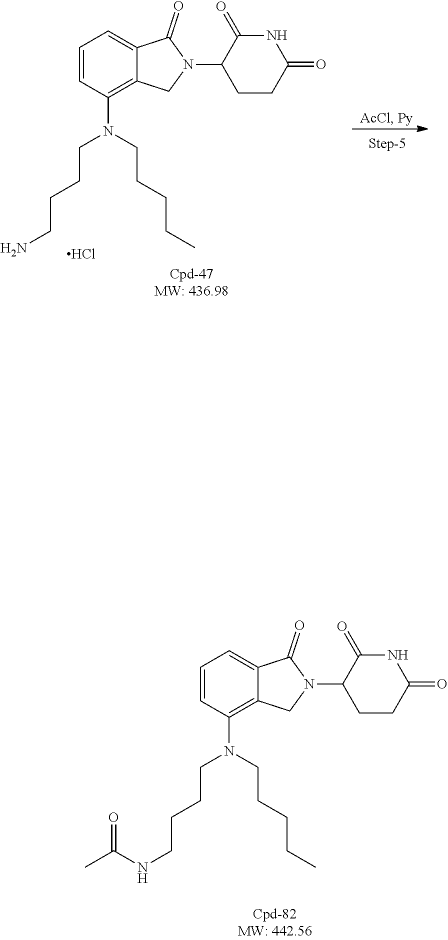 [SMILES: CCCCCN(CCCCN)c1cccc2c1CN(C1CCC(=O)NC1=O)C2=O ; "AcCl, Py" ; Cl ; CCCCCN(CCCCNC(C)=O)c1cccc2c1CN(C1CCC(=O)NC1=O)C2=O]

To a solution of 3-(4-((4-aminobutyl)(pentyl)amino)-1-oxoisoindolin-2-yl)piperidine-2,6-dione 47 (200 mg, 0.499 mmol) in pyridine (3 ml) was added acetic anhydride (0.236 ml, 2.497 mmol, 5 equiv) dropwise and the reaction mixture was stirred for 13 h at room temperature. Upon completion of the reaction (as confirmed by TLC analysis, 10% MeOH in DCM, R_{f}˜0.8, and LCMS), the reaction mixture was concentrated under vacuum, diluted with EtOAc (10 mL), washed with water (2×20 mL), brine (20 mL), dried over anhy. Na₂SO₄, filtered and concentrated. The resulted residue was purified by chromatography (Biotage R snap cartridge, KP-Sil, 25 g, 230-400 silica gel) with 3-4% MeOH in DCM and lyophilised to get N-(4-((2-(2,6-dioxopiperidin-3-yl)-1-oxoisoindolin-4-yl)(pentyl)amino)butyl)acetamide 82 (120 mg, 0.267 mmol, 53.5% yield) as off-white solid.

¹H-NMR (400 MHz, DMSO-d₆): δ 11.00 (s, 1H), 7.78 (t, J=5.60 Hz, 1H), 7.38 (t, J=7.60 Hz, 1H), 7.19 (d, J=7.20 Hz, 1H), 7.07 (d, J=8.00 Hz, 1H), 5.10 (t, J=5.20 Hz, 1H), 4.40 (d, J=16.80 Hz, 1H), 4.29 (d, J=16.80 Hz, 1H), 3.17 (t, J=8.40 Hz, 4H), 2.95 (q, J=1.60 Hz, 3H), 2.62-2.53 (m, 1H), 2.48 (s, 1H), 2.02 (d, J=5.20 Hz, 1H), 1.76 (s, 3H), 1.47-1.38 (m, 6H), 1.28-1.22 (m, 4H), 0.84 (t, J=6.80 Hz, 3H).

LCMS: 443.3 (M+H). Method: Mobile Phase A: 0.1% TFA in H₂O. Mobile Phase B: 0.1% TFA in ACN. Flow rate: 1.5 ml/min. Column: XBridge C8 (50×4.6 mm) 3.5 μm. Rt (min): 1.414; Area %: 98.439.

HPLC: Method: Mobile Phase A: 0.1% TFA in H₂O. Mobile Phase B: Acetonitrile. Flow rate: 2.0 mL/min. Column: X-Bridge C8(50×4.6) mm, 3.5 μm. Rt (min): 2.445; Area %: 98.636.

### Hydrochloride Salt Form of Compounds 87

### 3-(4-(((1S,4S)-4-aminocyclohexyl)(pentyl)amino)-1-oxoisoindolin-2-yl)piperidine-2,6-dione hydrochloride

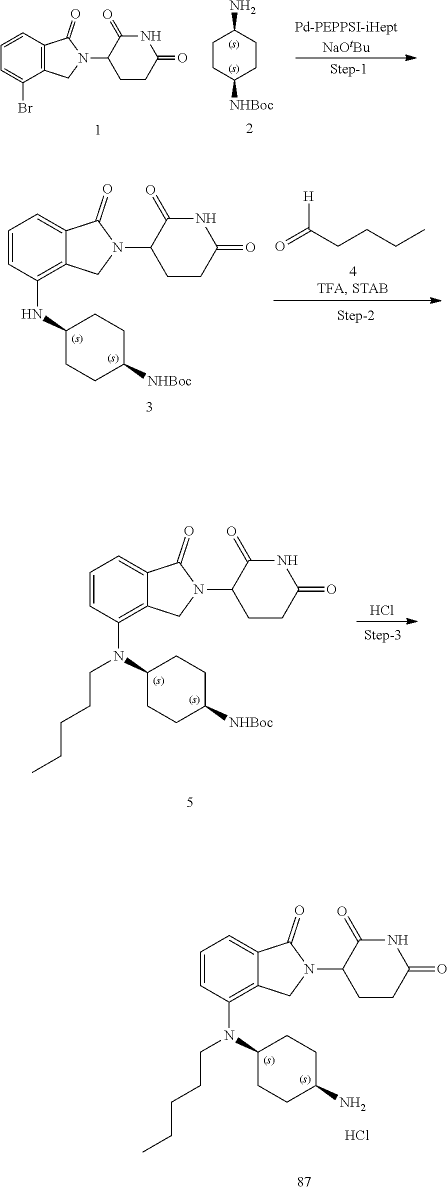

### Step-1: tert-butyl ((1S,4S)-4-((2-(2,6-dioxopiperidin-3-yl)-1-oxoisoindolin-4-yl)amino)cyclohexyl)carbamate

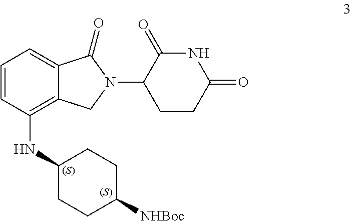

3-(4-bromo-1-oxoisoindolin-2-yl)piperidine-2,6-dione 1 (0.5 g, 1.547 mmol, 1 equiv), tert-butyl ((1S,4S)-4-aminocyclohexyl)carbamate 2 (0.995 g, 4.64 mmol, 3 equiv) and NaOt-Bu (0.446 g, 4.64 mmol, 3 equiv) were dissolved in DMF (2 ml) and de-gassed for 20 min with nitrogen. Pd-PEPPSI-IHept(Cl) (0.075 g, 0.077 mmol, 0.05 equiv) was added under nitrogen atmosphere and heated the reaction at 110° C. for 4 h. Upon completion of the reaction (as confirmed by TLC analysis, 100% EtOAc R_{f}˜0.4, and LCMS), the reaction mixture was filtered through Celite bed, washed with THF (10 mL), concentrated to minimum volume (3 mL) and purified by reverse-phase column chromatography (Grace® column: C18 40 μm, 120 g; flow rate: 20 mL/min; 0.1% aqueous HCOOH/ACN mobile phase) to obtain tert-butyl ((1S,4S)-4-((2-(2,6-dioxopiperidin-3-yl)-1-oxoisoindolin-4-yl)amino)cyclohexyl)carbamate 3 (110 mg, 0.240 mmol, 15% yield) as pale yellow solid.

LCMS: 401.1 (M-Boc). Method: Column: Atlantis dC18 (50×4.6 mm) 5 μm Mobile phase: A: 0.1% FA in H₂O B: ACN, Flow Rate: 1.5 mL/min. Rt (min): 2.110; Area %—94.51.

¹H-NMR (400 MHz, DMSO-d₆): δ 11.02 (s, 1H), 7.28 (t, J=7.60 Hz, 1H), 6.94 (d, J=6.80 Hz, 1H), 6.79 (d, J=8.00 Hz, 1H), 6.61 (m, 1H), 5.14 (q, J=5.20 Hz, 1H), 5.07 (d, J=6.40 Hz, 1H), 4.22 (q, J=17.20 Hz, 2H), 3.47-3.42 (m, 2H), 2.98-2.90 (m, 1H), 2.06-2.04 (m, 1H), 1.66 (m, 6H), 1.58-1.54 (m, 4H), 1.39 (s, 9H).

### Step-2: tert-butyl ((1S,4S)-4-((2-(2,6-dioxopiperidin-3-yl)-1-oxoisoindolin-4-yl)(pentyl)amino)cyclohexyl)carbamate

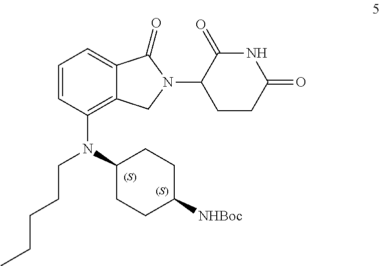

To a solution of tert-butyl ((1S,4S)-4-((2-(2,6-dioxopiperidin-3-yl)-1-oxoisoindolin-4-yl)amino)cyclohexyl)carbamate 3 (110 mg, 0.241 mmol, 1 equiv), pentanal 4 (0.128 ml, 1.205 mmol, 5 equiv) in DCM (4 ml) and DMF (4 ml) at 0° C. was added TFA (0.017 ml, 0.217 mmol, 0.9 equiv) followed bysodium triacetoxyborohydride (77.0 mg, 0.361 mmol, 1.5 equiv). The reaction mixture was warmed to RT and stirred at same temperature for 16 h. Upon completion of the reaction (as confirmed by TLC analysis, 100% EtOAc R_{f}˜0.5, and LCMS), the reaction mixture was diluted with ice-cold water (15 mL), extracted with ethyl acetate (2×15 mL), washed with brine (25 mL), dried over anhydrous Na₂SO₄, filtered and concentrated. The resulted residue was purified by reverse-phase column chromatography (Grace® column: C18 40 μm, 40 g; flow rate: 20 mL/min; 0.1% aqueous HCOOH/ACN mobile phase). The pure fractions from the column were lyophilized to afford tert-butyl ((1S,4S)-4-((2-(2,6-dioxopiperidin-3-yl)-1-oxoisoindolin-4-yl)(pentyl)amino)cyclohexyl)carbamate 5 (74 mg, 0.140 mmol, 58% yield) as white solid.

LCMS: 527.3 (M+H). Method: Column: Atlantis dC18 (50×4.6 mm) 5 μm Mobile phase: A: 0.1% FA in H₂O B: ACN, Flow Rate: 1.5 mL/min. Rt (min): 2.644; Area %—99.9.

¹H-NMR (400 MHz, DMSO-d₆): δ 10.96 (s, 1H), 7.41 (t, J=7.60 Hz, 1H), 7.26 (q, J=7.60 Hz, 2H), 6.97 (d, J=6.40 Hz, 1H), 5.08 (q, J=5.20 Hz, 1H), 4.31 (q, J=17.20 Hz, 2H), 3.55 (s, 1H), 3.32-2.91 (m, 3H), 2.96-2.80 (m, 1H), 2.60-2.55 (m, 1H), 2.04-2.00 (m, 1H), 1.82-1.79 (m, 2H), 1.65-1.63 (m, 2H), 1.46-1.40 (m, 12H), 1.27 (m, 6H), 0.81 (t, J=7.20 Hz, 3H).

### Step-3: 3-(4-(((1S,4S)-4-aminocyclohexyl)(pentyl)amino)-1-oxoisoindolin-2-yl)piperidine-2,6-dione hydrochloride

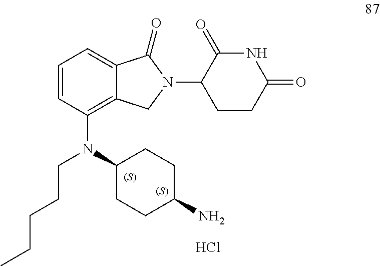

To an ice cold solution of tert-butyl ((1S,4S)-4-((2-(2,6-dioxopiperidin-3-yl)-1-oxoisoindolin-4-yl)(pentyl)amino)cyclohexyl)carbamate 5 (74 mg, 0.141 mmol, 1 equiv) in DCM (4 mL) was added HCl (4M soln. in EtOAc, 4 mL) and stirred for 1.5 h at room temperature. The progress of the reaction was monitored by LCMS. The reaction mixture was concentrated, washed with MTBE (2×5 mL) and further lyophilized to afford 3-(4-(((1S,4S)-4-aminocyclohexyl)(pentyl)amino)-1-oxoisoindolin-2-yl)piperidine-2,6-dione hydrochloride 87 (44 mg, 0.093 mmol, 97% yield) as off white solid.

LCMS: 427.2 (M-HCl). Method: Column: Atlantis dC18 (50×4.6 mm) 5 μm Mobile phase: A: 0.1% FA in H₂O B: ACN, Flow Rate: 1.5 mL/min. Rt (min): 1.634; Area %—85.00.

HPLC: 97.53%, Rt (min): 9.103. Method: Column: X-Bridge C8(50×4.6) mm, 3.5 μm, Mobile phase: A: 0.1% TFA in water, Mobile phase: B: ACN, Flow: 1.0 mL/min.

¹H-NMR (400 MHz, DMSO-d₆): δ 10.98 (s, 1H), 10.98 (s, 3H), 7.45-7.34 (m, 3H), 5.11 (dd, J=5.20, 13.20 Hz, 1H), 4.41-4.32 (m, 2H), 3.24 (m, 4H), 2.93-2.92 (m, 1H), 2.63-2.50 (m, 2H), 2.02 (t, J=6.80 Hz, 1H), 1.91-1.68 (m, 4H), 1.65-1.58 (m, 4H), 1.58-1.21 (m, 6H), 0.80 (t, J=6.80 Hz, 3H).

### Hydrochloride Salt Form of Compound 88

### 3-(4-(((1R,4R)-4-aminocyclohexyl)(pentyl)amino)-1-oxoisoindolin-2-yl)piperidine-2,6-dione hydrochloride

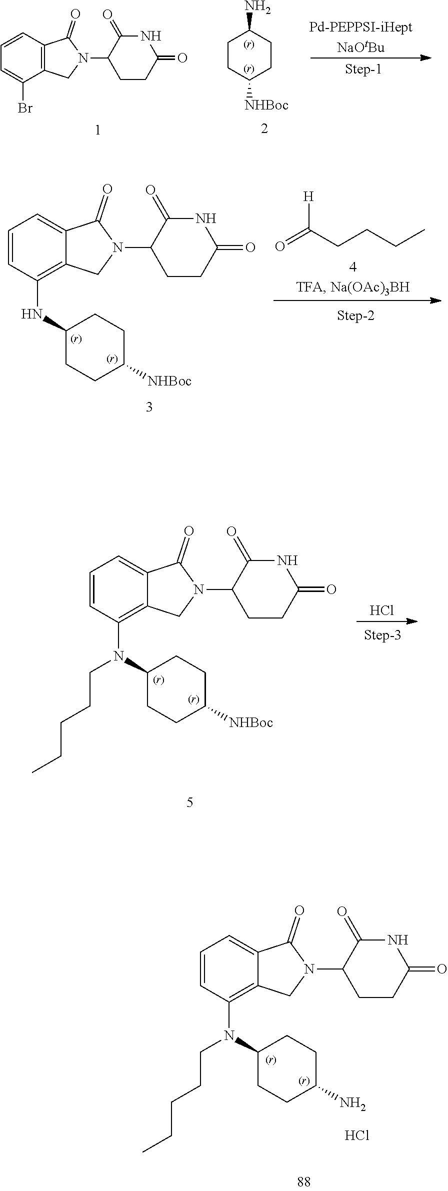

### Step-1: tert-butyl ((1R,4R)-4-((2-(2,6-dioxopiperidin-3-yl)-1-oxoisoindolin-4-yl)amino)cyclohexyl)carbamate

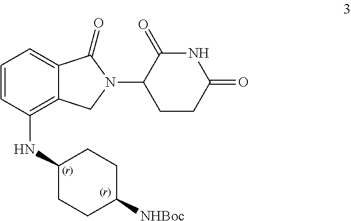

3-(4-bromo-1-oxoisoindolin-2-yl)piperidine-2,6-dione 1 (0.5 g, 1.547 mmol, 1 equiv), tert-butyl ((1R,4R)-4-aminocyclohexyl)carbamate 2 (0.995 g, 4.64 mmol, 3 equiv) and NaOt-Bu (0.446 g, 4.64 mmol, 3 equiv) were dissolved in DMF (2 ml) and de-gassed for 20 min with nitrogen. Pd-PEPPSI-IHept(Cl) (0.075 g, 0.077 mmol, 0.05 equiv) was added under nitrogen atmosphere and heated the reaction at 110° C. for 4 h. Upon completion of the reaction (as confirmed by TLC analysis, 100% EtOAc R_{f}˜0.4, and LCMS), the reaction mixture was filtered through celite, washed with THF (10 mL), concentrated to minimum volume (3 mL) and purified by reverse-phase column chromatography (Grace® column: C18 40 μm, 120 g; flow rate: 20 mL/min; 0.1% aqueous HCOOH/ACN mobile phase) to obtain tert-butyl ((1R,4S)-4-((2-(2,6-dioxopiperidin-3-yl)-1-oxoisoindolin-4-yl)amino)cyclohexyl)carbamate 3 (0.040 g, 0.086 mmol, 5% yield) as cream color solid.

LCMS: 401.1 (M-Boc). Method: Column: Atlantis dC18 (50×4.6 mm) 5 μm Mobile phase: A: 0.1% FA in H₂O B: ACN, Flow Rate: 1.5 mL/min. Rt (min): 2.188; Area %—98.18.

¹H-NMR (400 MHz, DMSO-d₆): δ 11.03 (s, 1H), 7.27 (t, J=7.20 Hz, 1H), 6.91 (d, J=7.20 Hz, 1H), 6.80 (t, J=8.00 Hz, 2H), 5.29 (d, J=6.80 Hz, 1H), 5.12 (q, J=4.80 Hz, 1H), 4.16 (q, J=17.20 Hz, 2H), 2.93-2.89 (m, 1H), 2.70-2.60 (m, 2H), 2.30-2.20 (m, 2H), 2.09-1.99 (m, 3H), 1.80 (m, 2H), 1.39 (s, 9H), 1.32-1.24 (m, 4H).

### Step-2: tert-butyl ((1R,4R)-4-((2-(2,6-dioxopiperidin-3-yl)-1-oxoisoindolin-4-yl)(pentyl)amino)cyclohexyl)carbamate

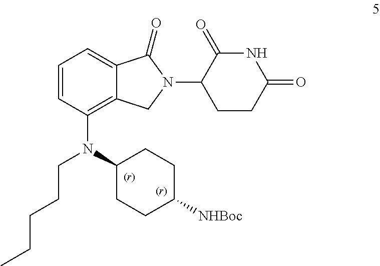

To a stirred solution of tert-butyl-((1R,4R)-4-((2-(2,6-dioxopiperidin-3-yl)-1-oxoisoindolin-4-yl)amino) cyclohexyl) carbamate 3 (0.040 g, 0.086 mmol, 1 equiv), pentanal (0.076 g, 0.876 mmol, 10 equiv) in DCM (1.5 ml) and DMF (1.5 ml) at 0° C. were added TFA (0.012 ml, 0.158 mmol, 1.8 equiv) and sodium triacetoxyborohydride (56 mg, 0.262 mmol, 3 equiv) and the reaction mixture was stirred at room temperature for 16 h. Upon completion of the reaction (as confirmed by TLC analysis, 100% EtOAc R_{f}˜0.5, and LCMS), the reaction mixture was diluted with ice-cold water (15 mL), extracted with ethyl acetate (2×15 mL), washed with brine (25 mL), dried over anhydrous Na₂SO₄, filtered and concentrated. The resulted residue was purified by reverse-phase column chromatography (Grace® column: C18 40 μm, 40 g; flow rate: 20 mL/min; 0.1% aqueous HCOOH/ACN mobile phase). The pure fractions from the column were lyophilized to afford tert-butyl ((1R,4R)-4-((2-(2,6-dioxopiperidin-3-yl)-1-oxoisoindolin-4-yl)(pentyl)amino)cyclohexyl)carbamate 5 (20 mg, 0.038 mmol, 43% yield) as white solid.

LCMS: 527.3 (M+H). Method: Column: Atlantis dC18 (50×4.6 mm) 5 μm Mobile phase: A: 0.1% FA in H₂O B: ACN, Flow Rate: 1.5 mL/min. Rt (min): 1.876; Area %—99.04.

### Step-3: 3-(4-(((1R,4R)-4-aminocyclohexyl)(pentyl)amino)-1-oxoisoindolin-2-yl)piperidine-2,6-dione hydrochloride

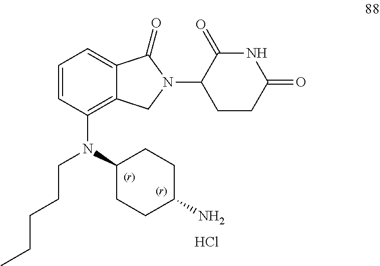

To an ice cold solution of tert-butyl ((1R,4R)-4-((2-(2,6-dioxopiperidin-3-yl)-1-oxoisoindolin-4-yl)(pentyl)amino)cyclohexyl)carbamate 5 (20 mg, 0.038 mmol, 1 equiv) in DCM (1 mL) was added HCl (4M soln. in EtOAc, 0.5 mL) and stirred for 3 h at room temperature. The progress of the reaction was monitored by LCMS. The reaction mixture was concentrated, washed with MTBE (2×5 mL) and lyophilized to afford 3-(4-(((1R,4R)-4-aminocyclohexyl)(pentyl)amino)-1-oxoisoindolin-2-yl)piperidine-2,6-dione hydrochloride 88 (17 mg, 0.036 mmol, 99% yield) as off white solid.

LCMS: 427.2 (M-HCl). Method: Column: Atlantis dC18 (50×4.6 mm) 5 μm Mobile phase: A: 0.1% FA in H₂O B: ACN, Flow Rate: 1.5 mL/min. Rt (min): 1.579; Area %—98.76.

HPLC: 99.10%, Rt (min): 2.381. Method: Column: X-Bridge C8(50×4.6) mm, 3.5 μm, Mobile phase: A: 0.1% TFA in water, Mobile phase: B: ACN, Flow: 2.0 mL/min.

¹H-NMR (400 MHz, DMSO-d₆): δ 10.98 (s, 1H), 7.91 (br s, 3H), 7.44-7.32 (m, 3H), 5.13 (dd, J=5.20, 13.20 Hz, 1H), 4.35-4.07 (m, 2H), 3.11-2.88 (m, 5H), 2.68-2.61 (m, 2H), 2.02-1.95 (m, 3H), 1.78 (s, 2H), 1.60-1.57 (m, 2H), 1.40-1.31 (m, 2H), 1.24-1.20 (m, 6H), 0.81-0.80 (m, 3H).

### Hydrochloride Salt Form of Compound 89

### 3-(4-(((1S,4S)-4-(aminomethyl)cyclohexyl)(pentyl)amino)-1-oxoisoindolin-2-yl)piperidine-2,6-dione hydrochloride

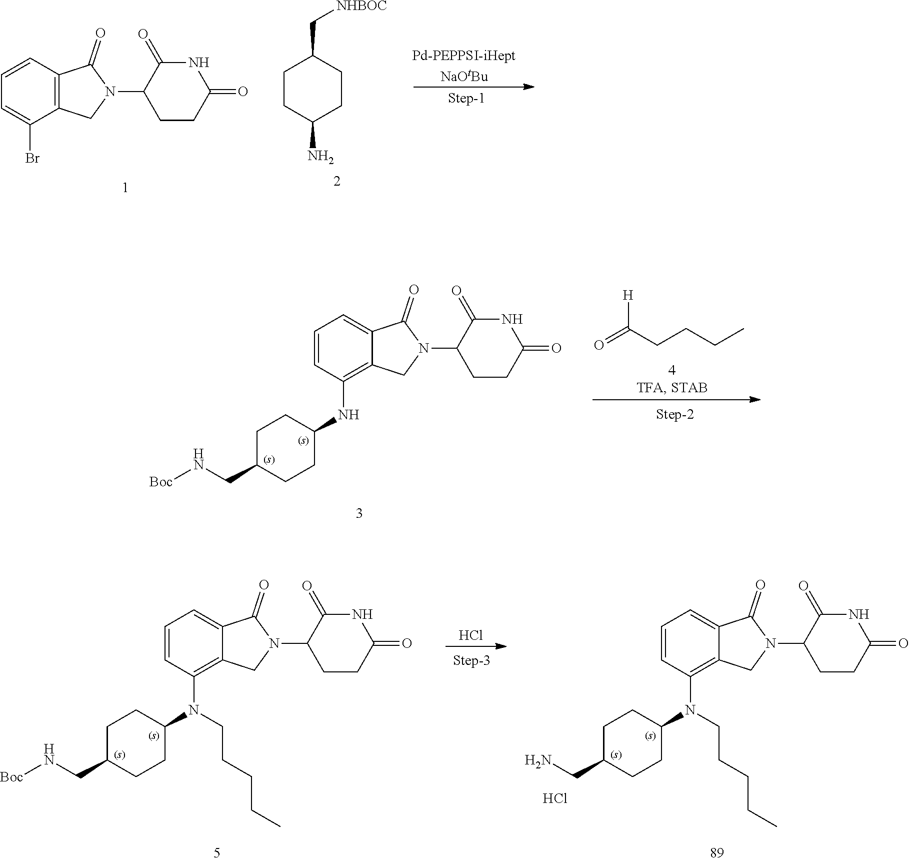

### Step-1: tert-butyl (((1S,4S)-4-((2-(2,6-dioxopiperidin-3-yl)-1-oxoisoindolin-4-yl)amino)cyclohexyl)methyl)carbamate

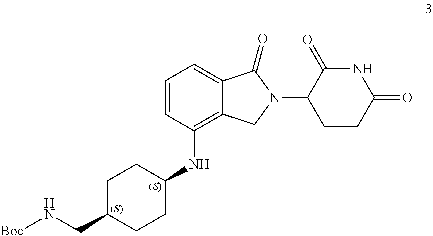

3-(4-bromo-1-oxoisoindolin-2-yl)piperidine-2,6-dione 1 (1.0 g, 3.09 mmol, 1 equiv), tert-butyl (((1s,4s)-4-aminocyclohexyl)methyl)carbamate 2 (1.060 g, 4.64 mmol, 1.5 equiv) and NaOt-Bu (0.892 g, 9.28 mmol, 3 equiv) were dissolved in DMF (5 ml) and de-gassed for 20 min with nitrogen. Pd-PEPPSI-IHept(Cl) (0.151 g, 0.155 mmol, 0.05 equiv) was added under nitrogen atmosphere and heated the reaction at 110° C. for 4 h. Upon completion of the reaction (as confirmed by TLC analysis, 100% EtOAc R_{f}˜0.4, and LCMS), the reaction mixture was filtered through Celite bed, washed with THF (10 mL), concentrated to minimum volume (3 mL) and purified by reverse-phase column chromatography (Grace® column: C18 40 μm, 120 g; flow rate: 20 mL/min; 0.1% aqueous HCOOH/ACN mobile phase) to obtain tert-butyl (((1S,4S)-4-((2-(2,6-dioxopiperidin-3-yl)-1-oxoisoindolin-4-yl)amino)cyclohexyl)methyl)carbamate 3 (140 mg, 0.272 mmol, 8.78% yield) as pale yellow solid.

LCMS: 415.1 (M-Boc). Method: Column: Atlantis dC18 (50×4.6 mm) 5 μm Mobile phase: A: 0.1% FA in H₂O B: ACN, Flow Rate: 1.5 mL/min. Rt (min): 2.325; Area % —89.09.

### Step-2: tert-butyl (((1s,4s)-4-((2-(2,6-dioxopiperidin-3-yl)-1-oxoisoindolin-4-yl)(pentyl)amino)cyclohexyl)methyl)carbamate

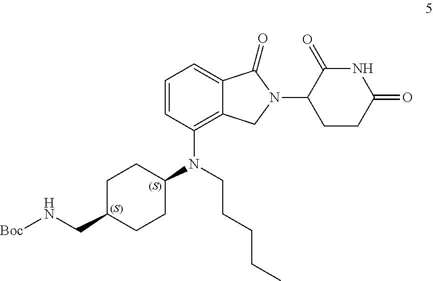

To a solution of tert-butyl (((1S,4S)-4-((2-(2,6-dioxopiperidin-3-yl)-1-oxoisoindolin-4-yl)amino)cyclohexyl)methyl)carbamate 3 (80 mg, 0.170 mmol, 1 equiv), pentanal 4 (73.2 mg, 0.850 mmol, 5 equiv) in DCM (2 ml) and DMF (2 ml) at 0° C. was added TFA (0.012 ml, 0.153 mmol, 0.9 equiv) followed bysodium triacetoxyborohydride (54 mg, 0.255 mmol, 1.5 equiv). The reaction mixture was warmed to RT and stirred at same temperature for 16 h. Upon completion of the reaction (as confirmed by TLC analysis, 100% EtOAc R_{f}˜0.5, and LCMS), the reaction mixture was diluted with ice-cold water (15 mL), extracted with ethyl acetate (2×15 mL), washed with brine (25 mL), dried over anhydrous Na₂SO₄, filtered and concentrated. The resulted residue was purified by reverse-phase column chromatography (Grace® column: C18 40 μm, 40 g; flow rate: 20 mL/min; 0.1% aqueous HCOOH/ACN mobile phase). The pure fractions from the column were lyophilized to afford tert-butyl (((1S,4S)-4-((2-(2,6-dioxopiperidin-3-yl)-1-oxoisoindolin-4-yl)(pentyl)amino)cyclohexyl)methyl)carbamate 5 (35 mg, 0.060 mmol, 35.5% yield) as off white solid.

LCMS: 541.3 (M+H). Method: Column: Atlantis dC18 (50×4.6 mm) 5 μm Mobile phase: A: 0.1% FA in H₂O B: ACN, Flow Rate: 1.5 mL/min. Rt (min): 2.648; Area %—93.13.

### Step-3: 3-(4-(((1S,4S)-4-(aminomethyl)cyclohexyl)(pentyl)amino)-1-oxoisoindolin-2-yl)piperidine-2,6-dione hydrochloride

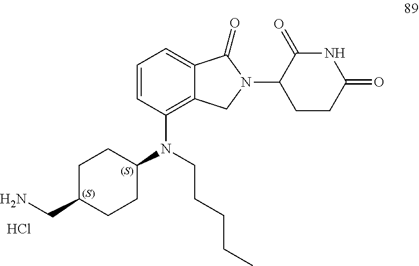

To an ice cold solution of tert-butyl (((1S,4S)-4-((2-(2,6-dioxopiperidin-3-yl)-1-oxoisoindolin-4-yl)(pentyl)amino)cyclohexyl)methyl)carbamate 5 (35 mg, 0.065 mmol, 1 equiv) in DCM (1 mL) was added HCl (4M soln. in EtOAc, 3 mL) and stirred for 1.5 h at room temperature. The progress of the reaction was monitored by LCMS. The reaction mixture was concentrated, washed with MTBE (2×5 mL) and lyophilized to afford 3-(4-(((1S,4S)-4-(aminomethyl)cyclohexyl)(pentyl)amino)-1-oxoisoindolin-2-yl)piperidine-2,6-dione hydrochloride 89 (22.5 mg, 0.045 mmol, 69% yield) as off white solid.

LCMS: 441.3 (M-HCl). Method: Column: Atlantis dC18 (50×4.6 mm) 5 μm Mobile phase: A: 0.1% TFA in H₂O B: 0.1% TFA in ACN, Flow Rate: 1.5 mL/min. Rt (min): 1.408; Area %—94.03.

HPLC: 94.36%, Rt (min): 8.083. Method: Column: X-Bridge C8(50×4.6) mm, 3.5 μm, Mobile phase: A: 0.1% TFA in water, Mobile phase: B: ACN, Flow: 1.0 mL/min.

¹H-NMR (400 MHz, DMSO-d₆): δ 10.98 (s, 1H), 7.84 (s, 3H), 7.34 (d, J=6.40 Hz, 3H), 5.11 (dd, J=4.80, 12.20 Hz, 1H), 4.38-4.24 (m, 2H), 3.19-3.10 (m, 3H), 2.92-2.84 (m, 3H), 2.69-2.68 (m, 2H), 1.95 (s, 1H), 1.75 (s, 1H), 1.60-1.50 (m, 4H), 1.49-1.30 (m, 4H), 1.24-1.20 (m, 6H), 0.80-0.78 (m, 3H).

### Hydrochloride Salt Form of Compound 90

### 3-(4-(((1R,4R)-4-(aminomethyl)cyclohexyl)(pentyl)amino)-1-oxoisoindolin-2-yl)piperidine-2,6-dione hydrochloride

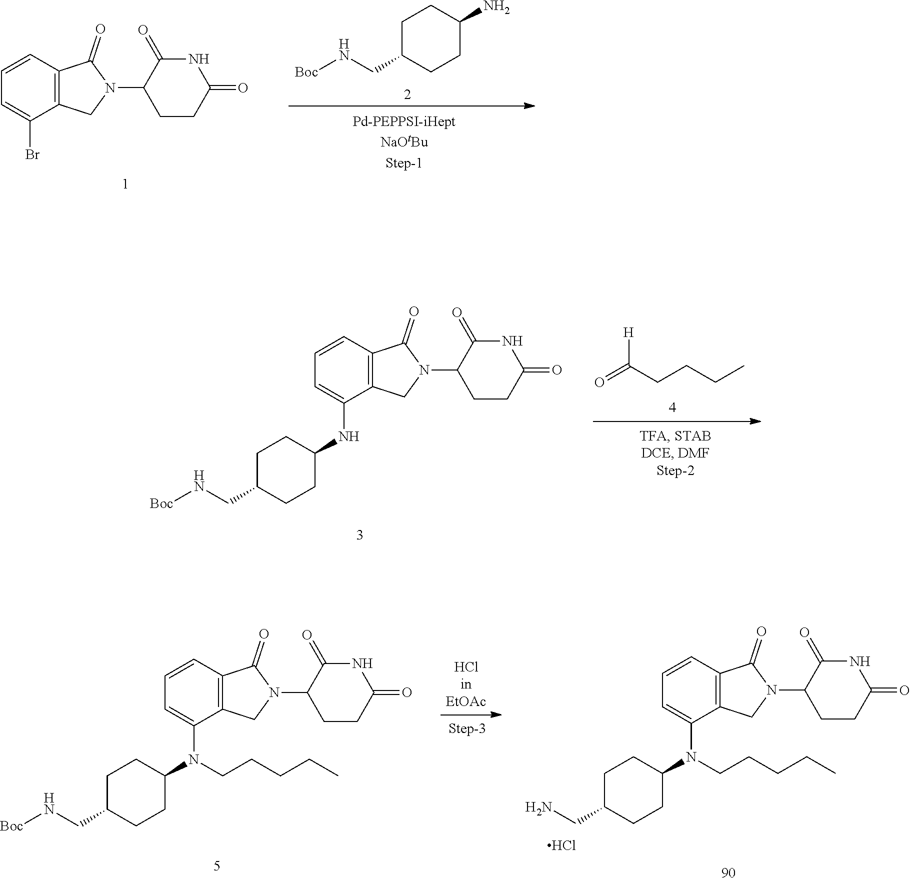

### Step-1: tert-butyl (((1R,4R)-4-((2-(2,6-dioxopiperidin-3-yl)-1-oxoisoindolin-4-yl)amino)cyclohexyl)methyl)carbamate

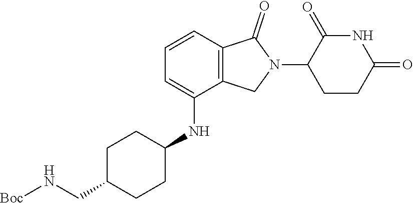 [SMILES: CC(C)(C)OC(=O)NC[C@H]1CC[C@H](Nc2cccc3c2CN(C2CCC(=O)NC2=O)C3=O)CC1]

To a solution of 3-(4-bromo-1-oxoisoindolin-2-yl)piperidine-2,6-dione 1 (0.500 g, 1.547 mmol, 1 equiv) in DMF (2 ml), tert-butyl (((1r,4r)-4-aminocyclohexyl)methyl)carbamate 2 (1.060 g, 4.64 mmol, 3 equiv) and sodium tert-butoxide (0.446 g, 4.64 mmol, 3 equiv) were added at room temperature and degassed with nitrogen gas for a period of 15 min. To this reaction mixture, Pd-PEPPSI-Ihept(Cl) (0.075 g, 0.077 mmol), 0.05 equiv) was added under nitrogen condition, degassed for 5 min and stirred at 110° C. 4 h. Upon completion of the reaction (as confirmed by UPLC analysis), the reaction mixture was filtered through Celite bed and washed with CH₃CN (5 ml), the filtrate was concentrated under reduced pressure to afford brown thick liquid. The crude compound was purified by reverse phase chromatography (Grace® column: C18 40 μm, 120 g, product eluted with 44% of ACN in 0.1% HCOOH). The pure fraction was lyophilized to afford tert-butyl (((1R,4R)-4-((2-(2,6-dioxopiperidin-3-yl)-1-oxoisoindolin-4-yl)amino)cyclohexyl) methyl)carbamate 3 (0.100 g, 0.118 mmol, 7.60% yield) as a yellow solid.

LCMS: 415.2 (M-56). Method: Column: Xbridge C8 (50×4.6 mm) 3.5 μm, Mobile phase: A: 0.1% TFA in H₂O, Mobile phase: B: 0.1% TFA in CAN, Flow Rate: 1.5 ml/min, Area %—55.37%.

### Step-2: tert-butyl (((1R,4R)-4-((2-(2,6-dioxopiperidin-3-yl)-1-oxoisoindolin-4-yl)(pentyl)amino)cyclohexyl)methyl)carbamate

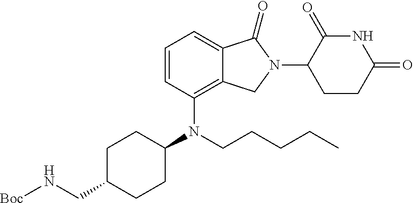 [SMILES: CCCCCN(c1cccc2c1CN(C1CCC(=O)NC1=O)C2=O)[C@H]1CC[C@H](CNC(=O)OC(C)(C)C)CC1]

A mixture of tert-butyl (((1R,4R)-4-((2-(2,6-dioxopiperidin-3-yl)-1-oxoisoindolin-4 yl)amino)cyclohexyl)methyl)carbamate 3 (0.100 g, 0.213 mmol, 1.0 equiv) and pentanal 4 (0.183 g, 2.125 mmol, 10 equiv) in DCE (1.5 ml) and DMF (1.5 ml) was added TFA (0.033 ml, 0.425 mmol) 2.0 equiv) and stirred for 10 min at 0° C. To this mixture, sodium triacetoxyborohydride (0.135 g, 0.638 mmol, 3.0 equiv) was added in portions at 0° C. and the resulted colourless cloudy mass was stirred for 16 h at room temperature (monitored by UPLC). Reaction mixture was quenched with ice cold water (5 ml) and extracted with dichloromethane (3×10 ml). The combined organic layer was dried over Na₂SO₄ and concentrated under vacuum to afford crude as a pale yellow liquid. The crude compound was purified by reverse phase chromatography (eluted with 50% of ACN in 0.1% HCOOH) to afford tert-butyl (((1r,4r)-4-((2-(2,6-dioxopiperidin-3-yl)-1-oxoisoindolin-4yl)(pentyl)amino)cyclohexyl) methyl)carbamate (0.020 g, 0.037 mmol, 17.18% yield) as a white solid.

¹H-NMR (400 MHz, DMSO-d₆): δ 11.06 (s, 1H), 7.81 (t, J=37.20 Hz, 1H), 7.24 (d, J=6.00 Hz, 2H), 5.26 (d, J=Hz, 1H), 4.72 (t, J=6.00 Hz, 2H), 3.35 (d, J=19.60 Hz, 3H), 2.75 (d, J=6.40 Hz, 3H), 2.33 (d, J=1.60 Hz, 2H), 1.71 (d, J=12.00 Hz, 5H), 1.42-1.37 (m, 14H), 0.79 (s, 3H).

LCMS: 541.4 (M+H). Method: Column: XBridge C8 (50×4.6 mm) 3.5 μm Mobile phase: A: 0.1% TFA in H₂O Mobile phase: B: 0.1% TFA in ACN Flow Rate: 1.5 ml/min Area %—94.78%.

HPLC: Method: Column: X-Bridge C8(50×4.6) mm, 3.5 μm Mobile phase: A: 0.1% TFA in water Mobile phase: B: ACN, Flow: 2.0 mL/min, Area % —98.72%.

### Step-3: 3-(4-(((1R,4R)-4-(aminomethyl)cyclohexyl)(pentyl)amino)-1-oxoisoindolin-2-yl)piperidine-2,6-dione hydrochloride

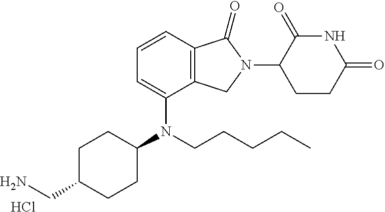 [SMILES: CCCCCN(c1cccc2c1CN(C1CCC(=O)NC1=O)C2=O)[C@H]1CC[C@H](CN)CC1 ; Cl]

To a stirred solution of tert-butyl (((1r,4r)-4-((2-(2,6-dioxopiperidin-3-yl)-1-oxoisoindolin-4-yl)(pentyl)amino)cyclohexyl)methyl)carbamate 5 (20 mg, 0.037 mmol) 1.0 equiv) in dichloromethane (1 ml) was added HCl (4M soln. in dioxane, 0.5 ml) at 0° C. and then stirred at room temperature for 2 h. The progress of the reaction was monitored by TLC (10% MeOH; DCM, R_{f}˜0.0. Reaction mixture was concentrated under vacuum at below 40° C., to afford off-white solid. The obtained solid was dissolved in water (5 ml) and washed with MTBE (2×5 ml), the water layer was lyophilized to 3-(4-(((1R,4R)-4-(aminomethyl)cyclohexyl)(pentyl)amino)-1-oxoisoindolin-2-yl)piperidine-2,6-dione hydrochloride (17 mg, 0.035 mmol, 96% yield) as off-white solid.

¹H-NMR (400 MHz, DMSO-d₆): δ 10.99 (s, 1H), 7.86 (br s, 3H), 7.43-7.29 (m, 3H), 5.12 (d, J=8.80 Hz, 1H), 4.33-4.27 (m, 2H), 3.50-3.30 (m, 2H), 3.12-3.08 (m, 2H), 2.96-2.87 (m, 1H), 2.68-2.59 (m, 3H), 2.04-2.02 (m, 1H), 1.80-1.75 (m, 4H), 1.50-1.48 (m, 3H), 1.21 (s, 6H), 1.00-0.97 (m, 2H), 0.79 (t, J=6.40 Hz, 3H).

LCMS: 441.3 (M+H). Method: Column: Atlantis dC18 (50×4.6 mm) 5 μm, Mobile phase: A: 0.1% FA in H₂O, Mobile phase: B: ACN, Flow Rate: 1.5 ml/min, Rt-1.526 min, Area: 97.05%.

HPLC: Method: Column: X-Bridge C8(50×4.6) mm, 3.5 μm, Mobile phase: A: 0.1% TFA in water, Mobile phase: B: ACN, Flow: 2.0 mL/min, Rt-2.287 min, Area:99.27%.

### Hydrochloride Salt Form of Compound 91

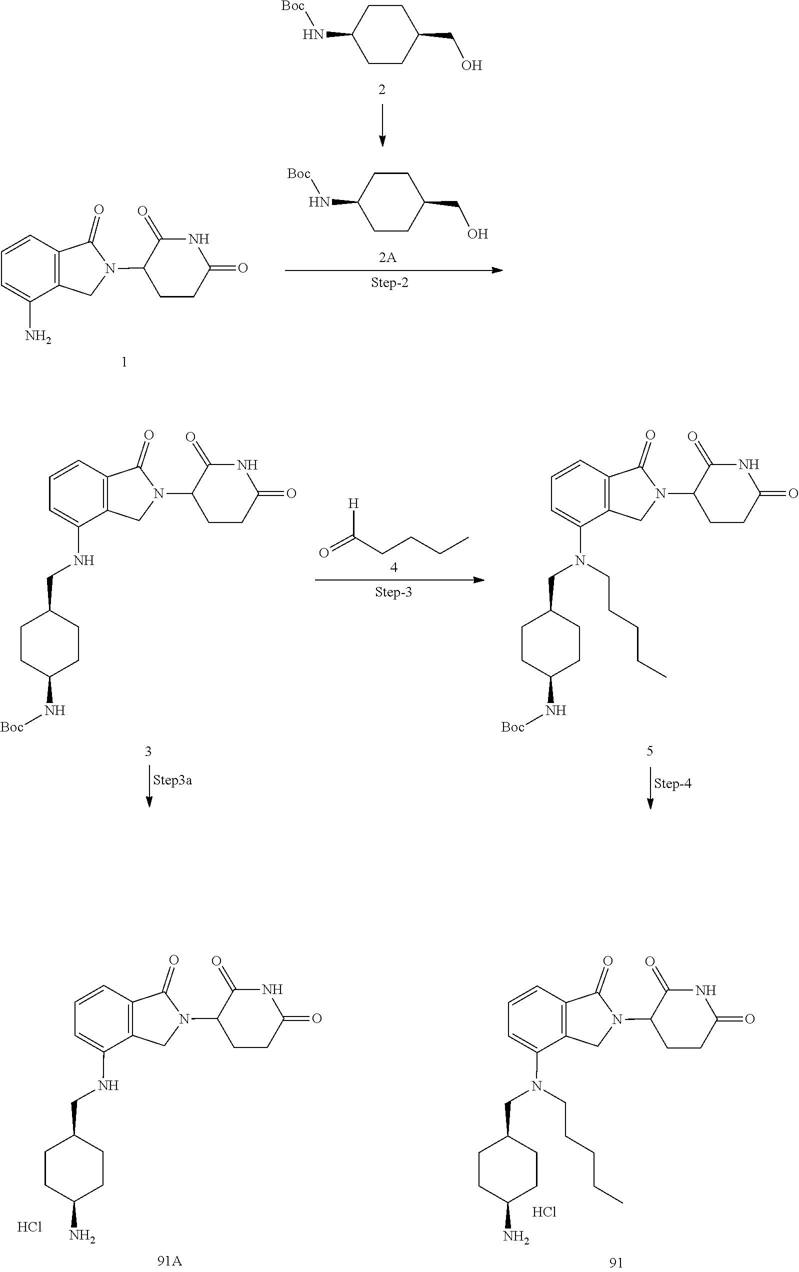

### Step-1: tert-butyl ((1s,4s)-4-formylcyclohexyl)carbamate

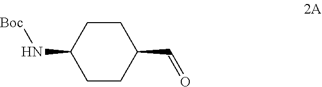 [SMILES: CC(C)(C)OC(=O)N[C@H]1CC[C@@H](C=O)CC1]

To a cooled solution of tert-butyl ((1s,4s)-4-(hydroxymethyl)cyclohexyl)carbamate (200 mg, 0.872 mmol, 1 equiv) in DCM (5 ml) was added DMSO (1.5 ml), DIPEA (438 mg, 3.39 mmol, 3.89 equiv) and a solution of pyridine sulfur trioxide (540 mg, 3.39 mmol, 3.89 equiv) in DMSO (1.5 ml) in drops and was stirred at 0° C. for 1 h. Upon completion of reaction (as confirmed by TLC (40% ethyl acetate in pet ether, R_{f}: 0.4), the reaction was quenched with 1.5N HCl (20 mL) and extracted with DCM (2×20 mL). The combined organic layer was dried over anhy. Na₂SO₄ and concentrated under vacuum to give the crude product of tert-butyl ((1s,4s)-4-formylcyclohexyl)carbamate 2A (200 mg, 0.880 mmol, 101% yield) as a colorless liquid.

### Step-2: tert-butyl((1s,4s)-4-(((2-(2,6-dioxopiperidin-3-yl)-1-oxoisoindolin-4-yl)amino)methyl)cyclohexyl)carbamate

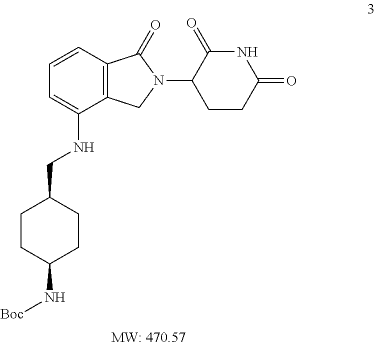 [SMILES: CC(C)(C)OC(=O)N[C@H]1CC[C@@H](CNc2cccc3c2CN(C2CCC(=O)NC2=O)C3=O)CC1]

To a solution of 3-(4-amino-1-oxoisoindolin-2-yl)piperidine-2,6-dione 1 (200 mg, 0.771 mmol, 1 equiv) and tert-butyl ((1s,4s)-4-formylcyclohexyl)carbamate 2A (210 mg, 0.926 mmol, 5 equiv) in DCE (2 ml) and DMF (0.5 ml) mixture was added TFA (79 mg, 0.694 mmol, 0.9 equiv) and Na(OAc)₃BH (245 mg, 1.157 mmol, 1.5 equiv) at 0° C. and was allowed to stir at room temperature for 12 h. Upon completion of the reaction (as confirmed by UPLC) the reaction was quenched with ice water (25 mL) and extracted with DCM (2×10 ml). The combined organic layer was concentrated under vacuum to give the crude product which was purified by reverse phase chromatography (Grace® column: generic C18, 40 g snap, 0.1% formic acid in ACN, 20 mL/min, 70-75% ACN in H₂O) to give tert-butyl ((1s,4s)-4-(((2-(2,6-dioxopiperidin-3-yl)-1-oxoisoindolin-4-yl)amino)methyl)cyclohexyl)carbamate 3 (105 mg, 0.223 mmol, 28.9% yield) as an off white solid.

¹H-NMR (400 MHz, DMSO-d6): δ 11.03 (s, 1H), 7.28 (t, J=8.00 Hz, 1H), 6.91 (d, J=7.20 Hz, 1H), 6.73 (d, J=8.00 Hz, 2H), 5.64 (t, J=5.20 Hz, 1H), 5.12 (q, J=5.20 Hz, 1H), 4.23 (d, J=17.20 Hz, 1H), 4.12 (d, J=17.20 Hz, 1H), 3.49 (s, 1H), 3.03 (t, J=6.00 Hz, 2H), 2.92 (t, J=8.00 Hz, 1H), 2.06-2.02 (m, 1H), 1.69 (s, 1H), 1.50 (t, J=13.20 Hz, 8H), 1.39 (s, 9H).

LCMS: 470.0 (M+H), Rt (min): 2.306, Area %: 99.775.

HPLC: Rt (min): 4.229, Area %: 99.789.

### Step-3: tert-butyl ((1s,4s)-4-(((2-(2,6-dioxopiperidin-3-yl)-1-oxoisoindolin-4-yl)(pentyl)amino)methyl)cyclohexyl)carbamate

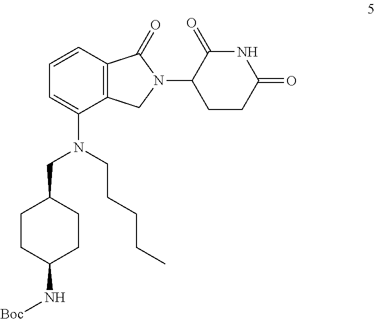 [SMILES: CCCCCN(C[C@H]1CC[C@@H](NC(=O)OC(C)(C)C)CC1)c1cccc2c1CN(C1CCC(=O)NC1=O)C2=O]

To a solution of tert-butyl ((1s,4s)-4-(((2-(2,6-dioxopiperidin-3-yl)-1-oxoisoindolin-4-yl)amino)methyl)cyclohexyl)carbamate (90 mg, 0.191 mmol, 1 equiv) in DCE (5 ml) and DMF (0.5 ml) was added pentanal (82 mg, 0.956 mmol, 5 equiv), TFA (19.63 mg, 0.172 mmol, 0.9 equiv) and Na(OAc)₃BH (48.6 mg, 0.230 mmol, 1.5 equiv) at 0° C. and was allowed to stir at room temperature for 12 h. Upon completion of the reaction (as confirmed by LCMS) the reaction was quenched with ice water (25 mL) and extracted with DCM (2×10 ml). The combined organic layer was concentrated under vacuum to give the crude product, which was purified by Isolera (column size: Biotage R snap cartridge, KP-Sil, 10 g, Silca gel—230-400 mesh) using 70-80% ethyl acetate in pet ether. Pure fractions were collected separately and concentrated under vacuum to give tert-butyl((1s,4s)-4-(((2-(2,6-dioxopiperidin-3-yl)-1-oxoisoindolin-4-yl)(pentyl)amino)methyl)cyclohexyl)carbamate (30 mg, 0.055 mmol, 29.0% yield) as white solid.

¹H-NMR (400 MHz, DMSO-d₆): δ 10.99 (s, 1H), 7.37 (t, J=7.60 Hz, 1H), 7.18 (d, J=7.20 Hz, 1H), 7.10 (d, J=8.00 Hz, 1H), 6.66 (d, J=7.20 Hz, 1H), 5.11 (q, J=4.80 Hz, 1H), 4.38 (d, J=16.80 Hz, 1H), 4.28 (d, J=17.20 Hz, 1H), 3.45 (s, 1H), 3.34-3.16 (m, 2H), 3.11 (d, J=7.20 Hz, 2H), 2.90 (d, J=12.00 Hz, 1H), 2.59 (d, J=19.60 Hz, 2H), 2.02 (t, J=5.20 Hz, 1H), 1.46 (t, J=7.20 Hz, 4H), 1.38 (s, 15H), 1.24 (t, J=10.00 Hz, 5H), 0.84 (t, J=6.80 Hz, 3H).

LCMS: 485.9 (M-56), Rt (min): 2.828, Area %: 99.796.

HPLC: Rt (min): 4.910, Area %: 98.887.

### Step-4: 3-(4-((((1s,4s)-4-aminocyclohexyl)methyl)(pentyl)amino)-1-oxoisoindolin-2-yl)piperidine-2,6-dione, HCl

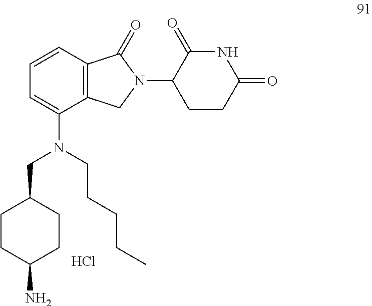 [SMILES: CCCCCN(C[C@H]1CC[C@@H](N)CC1)c1cccc2c1CN(C1CCC(=O)NC1=O)C2=O ; Cl]

To an ice cold solution of tert-butyl ((1s,4s)-4-(((2-(2,6-dioxopiperidin-3-yl)-1-oxoisoindolin-4-yl)(pentyl)amino)methyl)cyclohexyl)carbamate (30 mg, 0.055 mmol) in DCM (2 ml) was added HCl (4M in ethyl acetate) (0.5 mL, 16.46 mmol) at 0° C. and stirred for 6 h at room temperature. Upon completion of the reaction (as confirmed by LCMS), the reaction mixture was concentrated, washed with MTBE (10 mL) to give the crude which was lyophilised to get 3-(4-((((1s,4s)-4-aminocyclohexyl)methyl)(pentyl)amino)-1-oxoisoindolin-2-yl)piperidine-2,6-dione, HCl (30 mg, 0.063 mmol, 113% yield) as an off white solid.

¹H-NMR (400 MHz, DMSO-d₆): δ 11.00 (s, 1H), 7.78 (s, 6H), 7.39 (t, J=8.00 Hz, 1H), 7.22 (d, J=7.60 Hz, 1H), 7.22 (d, J=7.60 Hz, 1H), 5.12 (q, J=5.20 Hz, 1H), 4.33 (dd, J=16.80, 42.40 Hz, 2H), 3.15 (q, J=7.60 Hz, 5H), 2.05-2.01 (m, 1H), 1.59 (s, 5H), 1.45 (d, J=5.60 Hz, 6H), 1.25 (d, J=4.80 Hz, 4H), 0.84 (t, J=7.20 Hz, 3H).

LCMS: 442.0 (M+H), Method: Mobile phase: A: 0.1% Formic Acid in H₂O B: ACN, Column: Atlantis dC18 (50×4.6) 5μ, Flow Rate: 1.5 ml/min; Rt (min): 1.691, Area %: 99.845.

HPLC: Method: Mobile phase: A: 0.1% FA in H₂O, Mobile phase: B: ACN, Column: Atlantis dC18 (50×4.6 mm) 5 μm, Flow Rate: 1.5 ml/min, Rt (min): 2.839, Area %: 98.026.

### Hydrochloride Salt Form of Compound 92

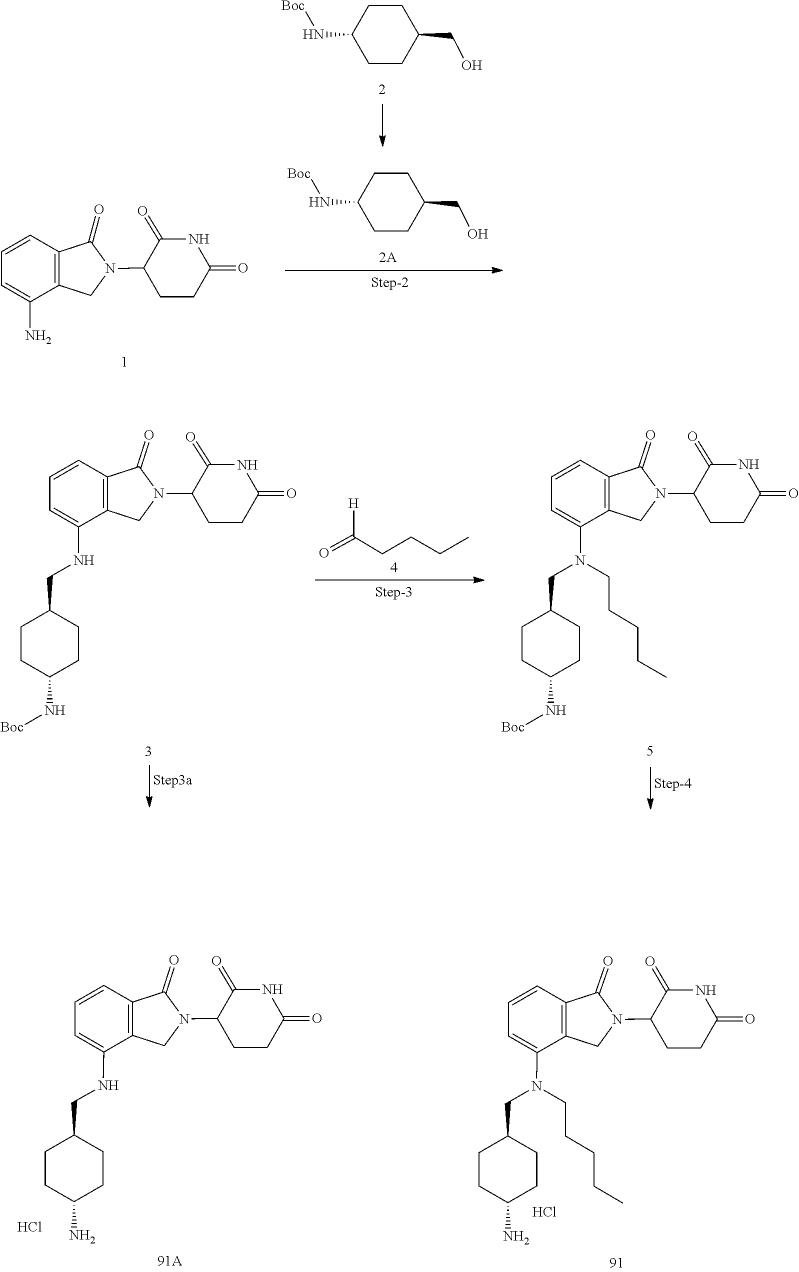

### Step-1: tert-butyl ((1r,4r)-4-formylcyclohexyl)carbamate

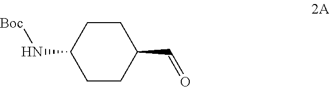 [SMILES: CC(C)(C)OC(=O)N[C@H]1CC[C@H](C=O)CC1]

To a cooled solution of tert-butyl ((1r,4r)-4-(hydroxymethyl)cyclohexyl)carbamate (500 mg, 2.180 mmol, 1 equiv) in DCM (11 ml) was added DMSO (3.7 ml), DIPEA (0.388 ml, 2.180 mmol, 3.89 equiv) and a solution of pyridine sulfur trioxide (347 mg, 2.180 mmol, 3.89 equiv) in DMSO (3.7 ml) in drops and was stirred at 0° C. for 1 h. Upon completion of reaction (as confirmed by TLC (40% ethyl acetate in pet ether, Rf: 0.4), the reaction was quenched with 1.5N HCl (20 mL) and extracted with DCM (2×20 mL). The combined organic layer was dried over anhy. Na2SO4 and concentrated under vacuum to give the crude product of tert-butyl ((1r,4r)-4-formylcyclohexyl)carbamate 2A (480 mg, 2.112 mmol, 97% yield) as a yellow liquid.

¹H-NMR (400 MHz, DMSO-d6): δ 9.55 (s, 1H), 3.64-3.50 (m, 1H), 3.16-3.11 (m, 2H), 2.16-2.11 (m, 1H), 1.99 (d, J=57.60 Hz, 2H), 1.88 (t, J=24.40 Hz, 3H), 1.38 (s, 10H), 1.18-1.26 (m, 11H).

LCMS: 172.4 (M-56), Rt (min): 2.593, Area %: 59.513.

### Step-2: tert-butyl((1r,4r)-4-(((2-(2,6-dioxopiperidin-3-yl)-1-oxoisoindolin-4-yl)amino)methyl)cyclohexyl)carbamate

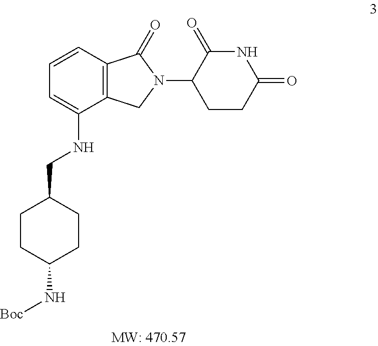 [SMILES: CC(C)(C)OC(=O)N[C@H]1CC[C@H](CNc2cccc3c2CN(C2CCC(=O)NC2=O)C3=O)CC1]

To a solution of 3-(4-amino-1-oxoisoindolin-2-yl)piperidine-2,6-dione 1 (350 mg, 1.350 mmol, 1 equiv) and tert-butyl ((1r,4r)-4-formylcyclohexyl)carbamate 2A (368 mg, 1.620 mmol, 5 equiv) in DCE (1.5 ml) and DMF (0.5 ml) mixture was added TFA (139 mg, 1.215 mmol, 0.9 equiv) and Na(OAc)₃BH (429 mg, 2.025 mmol, 1.5 equiv) at 0° C. and was allowed to stir at room temperature for 12 h. Upon completion of the reaction (as confirmed by TLC) the reaction was quenched with ice water (25 mL) and extracted with DCM (2×10 ml). The combined organic layer was concentrated under vacuum to give the crude product which was purified by reverse phase chromatography (Grace®, column: generic C18, 40 g snap, 0.1% formic acid in ACN, 20 mL/min, 70-75% ACN in H₂O) and the pure fraction was Lyophilised to give tert-butyl ((1r,4r)-4-(((2-(2,6-dioxopiperidin-3-yl)-1-oxoisoindolin-4-yl)amino)methyl)cyclohexyl)carbamate 3 (410 mg, 0.834 mmol, 61.8% yield) as white solid.

¹H-NMR (400 MHz, DMSO-d6): δ 11.00 (s, 1H), 7.27 (t, J=7.60 Hz, 1H), 6.91 (d, J=7.60 Hz, 1H), 6.71 (q, J=8.00 Hz, 2H), 5.62 (t, J=5.60 Hz, 1H), 5.12 (q, J=4.80 Hz, 1H), 4.23 (d, J=17.20 Hz, 1H), 4.12 (d, J=17.20 Hz, 1H), 3.19 (d, J=8.00 Hz, 1H), 2.98-2.89 (m, 3H), 2.60 (t, J=22.00 Hz, 2H), 2.35-2.28 (m, 2H), 2.06-2.02 (m, 1H), 1.81 (t, J=20.00 Hz, 4H), 1.50 (t, J=7.20 Hz, 2H), 1.37 (s, 9H), 1.12 (q, J=11.60 Hz, 3H), 0.99 (t, J=12.00 Hz, 2H),

LCMS: 415.3 (M-56), Rt (min): 2.127, Area %: 95.713.

HPLC: Rt (min): 4.211, Area %: 98.834.

### Step-3: tert-butyl ((1r,4r)-4-(((2-(2,6-dioxopiperidin-3-yl)-1-oxoisoindolin-4-yl)(pentyl)amino)methyl)cyclohexyl)carbamate

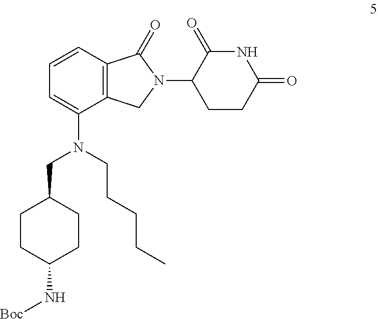 [SMILES: CCCCCN(C[C@H]1CC[C@H](NC(=O)OC(C)(C)C)CC1)c1cccc2c1CN(C1CCC(=O)NC1=O)C2=O]

To a solution of tert-butyl ((1r,4r)-4-(((2-(2,6-dioxopiperidin-3-yl)-1-oxoisoindolin-4-yl)amino)methyl)cyclohexyl)carbamate (250 mg, 0.531 mmol, 1 equiv) in DCE (2.5 ml) and DMF (0.5 ml) was added pentanal (229 mg, 2.66 mmol, 5 equiv), TFA (54.5 mg, 0.478 mmol, 0.9 equiv) and Na(OAc)₃BH (169 mg, 0.797 mmol, 1.5 equiv) at 0° C. and was allowed to stir at room temperature for 12 h. Upon completion of the reaction (as confirmed by LCMS), the reaction was quenched with ice water (25 mL) and extracted with DCM (2×10 ml). The combined organic layer was concentrated under vacuum to give the crude product which was purified by Isolera (column size: Biotage R snap cartridge, KP-Sil, 25 g, Silca gel—230-400 mesh) using 70-80% ethyl acetate in pet ether. Pure fractions were collected separately and concentrated under vacuum to afford tert-butyl((1r,4r)-4-(((2-(2,6-dioxopiperidin-3-yl)-1-oxoisoindolin-4-yl)(pentyl)amino)methyl) cyclohexyl)carbamate 5 (124 mg, 0.229 mmol, 43.2% yield) as yellow solid.

¹H-NMR (400 MHz, DMSO-d6): δ 10.97 (s, 1H), 7.37 (t, J=8.00 Hz, 1H), 7.13 (dd, J=8.00, 35.00 Hz, 2H), 6.63 (d, J=8.00 Hz, 1H), 5.09 (t, J=5.20 Hz, 1H), 4.31 (t, J=16.80 Hz, 2H), 3.31-3.15 (m, 3H), 3.05-3.03 (m, 2H), 2.00 (s, 1H), 1.72 (d, J=9.60 Hz, 5H), 1.43-1.36 (m, 14H), 1.26-1.20 (m, 5H), 0.97 (d, J=27.20 Hz, 4H), 0.83 (t, J=6.80 Hz, 3H).

LCMS: 541.3 (M+H), Rt (min): 2.849, Area %: 95.954.

HPLC: Rt (min): 4.907, Area %: 95.462.

### Step-4: 3-(4-((((1r,4r)-4-aminocyclohexyl)methyl)(pentyl)amino)-1-oxoisoindolin-2-yl)piperidine-2,6-dione, HCl

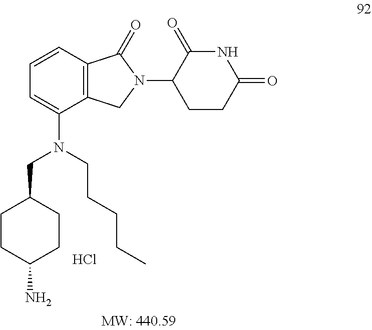 [SMILES: CCCCCN(C[C@H]1CC[C@H](N)CC1)c1cccc2c1CN(C1CCC(=O)NC1=O)C2=O ; Cl]

To an ice cold solution of tert-butyl ((1r,4r)-4-(((2-(2,6-dioxopiperidin-3-yl)-1-oxoisoindolin-4-yl)(pentyl)amino)methyl)cyclohexyl)carbamate (120 mg, 0.222 mmol) in DCM (5 ml) was added HCl (4M in ethyl acetate) (0.12, 3.95 mmol) at 0° C. and stirred for 6 h at room temperature. Upon completion of the reaction (as confirmed by LCMS), the reaction mixture was concentrated, washed with MTBE (10 mL) and dried to 3-(4-((((1r,4r)-4-aminocyclohexyl)methyl)(pentyl)amino)-1-oxoisoindolin-2-yl)piperidine-2,6-dione, HCl 92 (30 mg, 0.061 mmol, 27.3% yield) as pale yellow solid.

¹H-NMR (400 MHz, DMSO-d6): δ 11.00 (s, 1H), 7.83 (s, 3H), 7.39 (t, J=7.60 Hz, 1H), 7.20 (d, J=7.20 Hz, 1H), 7.11 (d, J=8.00 Hz, 1H), 5.11 (q, J=4.80 Hz, 1H), 4.39 (d, J=16.80 Hz, 1H), 4.28 (d, J=16.80 Hz, 1H), 3.18 (d, J=4.80 Hz, 2H), 3.18 (d, J=4.80 Hz, 2H), 2.93-2.88 (m, 2H), 2.68 (t, J=2.00 Hz, 1H), 2.62 (d, J=2.80 Hz, 1H), 2.02 (d, J=5.60 Hz, 1H), 1.90 (d, J=10.40 Hz, 2H), 1.77 (d, J=12.00 Hz, 2H), 1.41 (t, J=6.80 Hz, 3H), 1.17-1.20 (m, 6H), 0.97 (d, J=12.00 Hz, 2H), 0.83 (t, J=6.80 Hz, 3H).

LCMS: 441.3 (M+H), Method: Mobile phase: A: 0.1% FA in H₂O, Mobile phase: B: ACN, Column: Atlantis dC18 (50×4.6 mm) 5 μm, Flow Rate: 1.5 ml/min, Rt (min): 1.695, Area %: 96.479.

HPLC: Method: A: 0.1% TFA in H₂O, B: ACN, Flow Rate: 2.0 mL/min COLUMN: Xbridge C8(50×4.6) mm, 3.5 μm, Rt (min): 2.950, Area %: 95.528.

### Hydrochloride Salt Form of Compound 93

### (E)-3-(4-((4-aminobutyl)(pent-3-en-1-yl)amino)-1-oxoisoindolin-2-yl)piperidine-2,6-dione hydrochloride

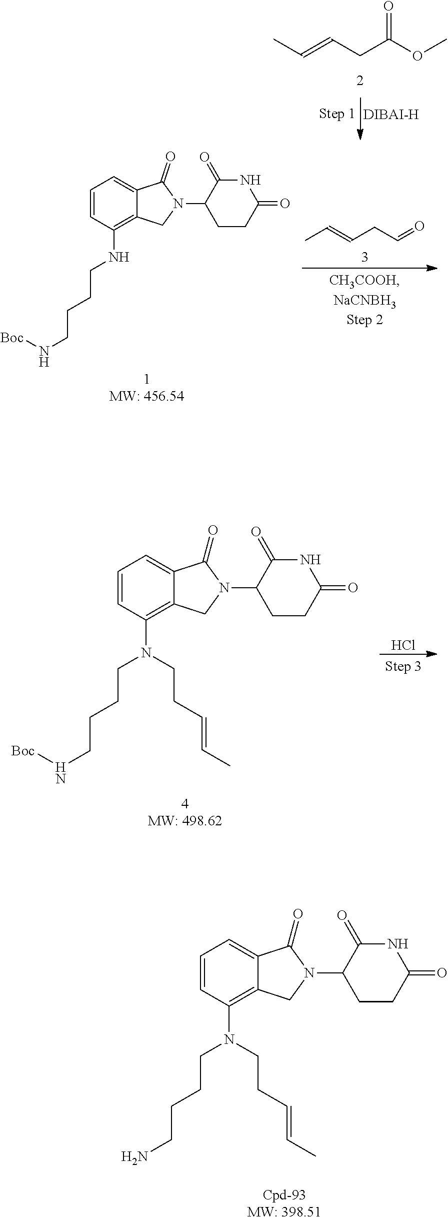

### Step-1: (E)-pent-3-enal

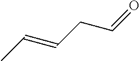

To a stirred solution of methyl (E)-pent-3-enoate 2 (2.5 g, 21.90 mmol, 1.0 equiv) in CH₂Cl₂ (50 ml) was added DIBAL-H in hexane (17.52 ml, 17.52 mmol, 0.8 equiv) in drops at −78° C. Then the resulting reaction mixture was stirred at 78° C. for 5 min. Upon completion of the reaction (as confirmed by TLC analysis, 20% EtOAc in pet ether, R_{f}˜0.5), the reaction mixture was quenched with 1.5 N HCl solution (10 ml) and extracted with DCM (20 mL). The separated organic layer was washed with brine (20 mL), dried over Na₂SO₄, filtered and used for next step without concentration

### Step-2: tert-butyl (E)-(4-((2-(2,6-dioxopiperidin-3-yl)-1-oxoisoindolin-4-yl)(pent-3-en-1-yl)amino)butyl)carbamate

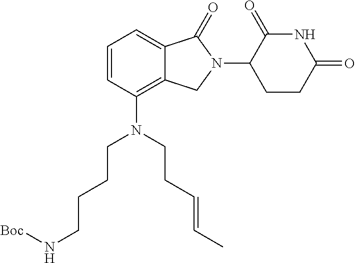 [SMILES: C/C=C/CCN(CCCCNC(=O)OC(C)(C)C)c1cccc2c1CN(C1CCC(=O)NC1=O)C2=O]

To a stirred solution of tert-butyl (4-((2-(2,6-dioxopiperidin-3-yl)-1-oxoisoindolin-4-yl)amino)butyl)carbamate 1 (0.5 g, 1.161 mmol, 1.0 equiv) and (E)-pent-3-enal 3 (1.954 g, 23.23 mmol, 20.0 equiv) in a mixture of CH₂Cl₂ (10 ml) and DMF (5 ml) was added AcOH (0.332 ml, 5.81 mmol, 5.0 equiv) under nitrogen atmosphere and stirred for 1 h. To this mixture, NaCNBH4 (0.292 g, 4.65 mmol, 4.0 equiv) was added at 0° C. The resulting reaction mixture was stirred for over night at room temperature. Upon completion of the reaction (as confirmed by TLC and UPLC analysis, 100% EtOAc, R_{f}˜0.7), the reaction mixture was diluted with DCM (30 mL) and washed with water (2×30 mL), 10% LiCl solution (30 mL) and brine (30 mL), dried over Na₂SO₄, filtered and concentrated to afford crude product as pale yellow liquid. The crude compound was purified by prep-HPLC (Column: X-Bridge-c18 19.1×250, Mobile phase: 0.1% FA in Water/ACN, FLOW rate:15 ml/min) to afford tert-butyl (E)-(4-((2-(2,6-dioxopiperidin-3-yl)-1-oxoisoindolin-4-yl)(pent-3-en-1-yl)amino)butyl) carbamate 4 (35 mg, 0.068 mmol, 5.87% yield) as a off-white solid.

¹H-NMR (400 MHz, DMSO-d6): δ 10.98 (s, 1H), 7.36-7.40 (m, 1H), 7.18-7.20 (m, 1H), 7.06-7.08 (m, 1H), 6.78 (s, 1H), 5.40-5.42 (m, 3H), 5.09-5.13 (m, 1H), 4.26-4.42 (m, 2H), 3.41-3.51 (m, 3H), 3.19-3.22 (m, 4H), 2.88-2.92 (m, 3H), 2.11-2.13 (m, 2H), 2.03 (m, 1H), 1.62-1.63 (m, 3H), 1.59-1.60 (m, 2H), 1.37 (s, 9H),

LCMS: 499.2 (M+H), Rt (min):2.492, Area—97%.

### Step-3: (E)-3-(4-((4-aminobutyl)(pent-3-en-1-yl)amino)-1-oxoisoindolin-2-yl)piperidine-2,6-dione hydrochloride

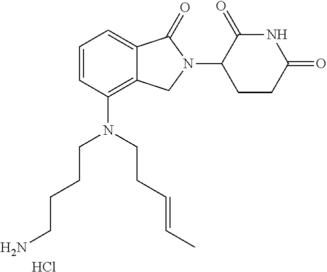 [SMILES: C/C=C/CCN(CCCCN)c1cccc2c1CN(C1CCC(=O)NC1=O)C2=O ; Cl]

To a stirred solution of tert-butyl (E)-(4-((2-(2,6-dioxopiperidin-3-yl)-1-oxoisoindolin-4-yl)(pent-3-en-1-yl)amino)butyl)carbamate 4 (65 mg, 0.130 mmol, 1.0 equiv) in ethyl acetate (1 ml) was added HCl (4M soln. in EtOAc, 2 ml) at 0° C. and then stirred at room temperature for 3 h. The progress of the reaction was monitored by TLC (100% EtOAc). Reaction mixture was concentrated under vacuum at below 40° C., to afford off-white semi solid. The obtained solid was dissolved in water (2 ml) and washed with MTBE (2 ml), and additional water (2 ml) was added and lyophilized to afford (E)-3-(4-((4-aminobutyl)(pent-3-en-1-yl)amino)-1-oxoisoindolin-2-yl)piperidine-2,6-dione, HCl (30 mg, 0.067 mmol, 79% yield) as a off-white solid.

¹H-NMR (400 MHz, DMSO-d6): δ 10.98 (s, 1H), 7.84 (s, 3H), 7.09-7.43 (m, 3H), 5.40-5.41 (m, 2H), 5.10-5.14 (m, 1H), 4.32-4.39 (m, 2H), 3.23-3.48 (m, 4H), 2.90-2.93 (m, 1H), 2.74-2.78 (m, 2H), 2.53-2.68 (m, 1H), 2.12-2.14 (m, 2H), 2.02-2.09 (m, 2H), 1.52-1.60 (m, 7H)

LCMS: 399.2 (M+H). Method: Column: Atlantis dC18 (50×4.6 mm) 5 μm, Mobile phase: A: 0.1% FA in H₂O, Mobile phase: B: Acetonitrile, Flow Rate: 1.5 ml/min

HPLC: Method: Mobile phase: A: 0.1% FA in water, Mobile phase: B: ACN, Flow: 2.0 mL/min, Column: X-Bridge C8(50×4.6) mm, 3.5 μm, RT: 2.301 min, Area: 97.652%.

### Trifluoroacetic Acid Salt Form of Compound 94

### (Z)-3-(4-((4-aminobutyl)(pent-3-en-1-yl)amino)-1-oxoisoindolin-2-yl)piperidine-2,6-dione

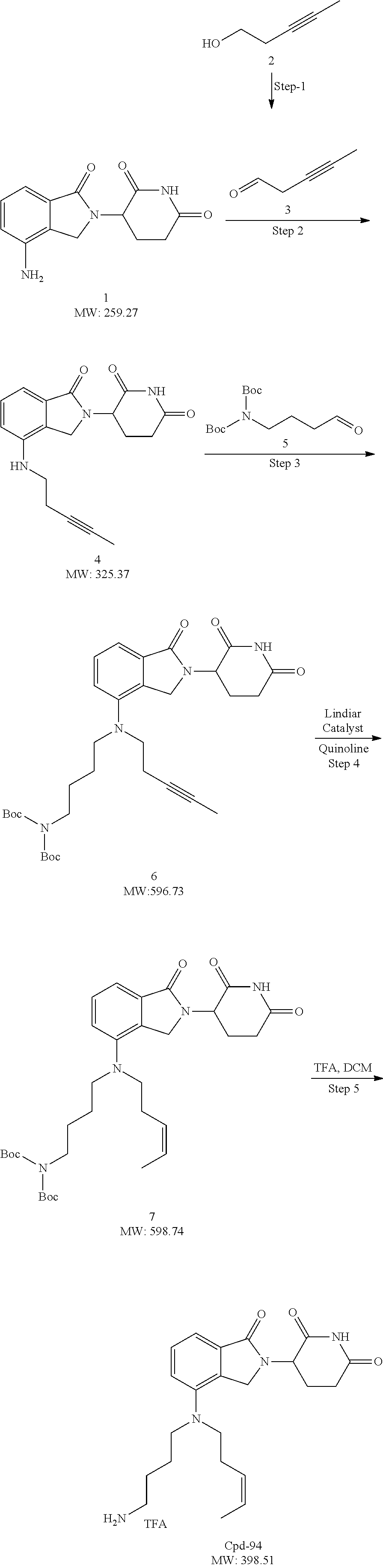

### Steps-1 & 2: (3-(1-oxo-4-(pent-3-yn-1-ylamino)isoindolin-2-yl)piperidine-2,6-dione

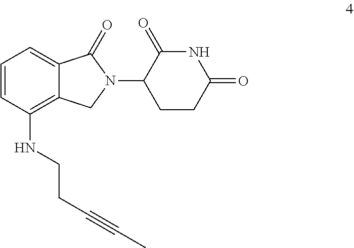 [SMILES: CC#CCCNc1cccc2c1CN(C1CCC(=O)NC1=O)C2=O]

To a solution of pent-3-yn-1-ol 2 (2.0 g, 23.78 mmol, 1.0 equiv) and CH₂Cl₂ (100 ml) was added Dess-Martin periodinane (11.09 g, 26.2 mmol, 1.1 equiv) and water (0.05 ml) at 0° C. and the resulted white suspension was stirred for 1 h at room temperature. Upon completion of the reaction (as confirmed by TLC, 20% EtOAc in pet-ether), the reaction mixture was cooled to −78° C. and diluted with n-pentane (50 ml) and filtered through silica gel bed. The bed was washed with n-pentane (20 ml) and the filtrate was concentrated to ˜50% of the quantity under vacuum at 25° C., the resulting solution of 3 was used directly for next step.

In another RB flask, to a stirred solution of 3-(4-amino-1-oxoisoindolin-2-yl)piperidine-2,6-dione 1 (4.00 g, 15.43 mmol, 0.649 equiv) in DMF (40 ml) was added the above solution of 3 at room temperature. Acetic acid (1.753 ml, 30.6 mmol, 1.289 equiv) was added to the reaction mixture and stirred for 2 hours at room temperature. Sodium cyanoborohydride (1.520 g, 24.18 mmol, 1.017 equiv) was added to the reaction mixture at 0° C. and stirred for over night at room temperature. Upon completion of the reaction (as confirmed by TLC and UPLC analysis, 100% EtOAc, R_{f}˜0.7), the reaction mixture was diluted with DCM (100 mL) and washed with water (2×50 mL), dried over Na₂SO₄, filtered and concentrated to afford crude product as pale yellow liquid. The crude compound was purified by reverse phase (Grace® column: C18 40 μm, 120 g; flow rate: 20 mL/min; 0.1% aqueous HCOOH/ACN mobile phase, product eluted with 70% of ACN in 0.1% HCO₂H) to afford 3-(1-oxo-4-(pent-3-yn-1-ylamino)isoindolin-2-yl)piperidine-2,6-dione 4 (1.0 g, 3.03 mmol, 12.75% yield) as a pale yellow solid.

¹H-NMR (400 MHz, DMSO-d6): δ 11.01 (s, 1H), 7.30 (t, J=8.00 Hz, 1H), 6.96 (d, J=7.20 Hz, 1H), 6.78 (d, J=6.00 Hz, 1H), 5.72 (t, J=6.00 Hz, 1H), 5.09-5.14 (m, 1H), 4.23 (d, J=16.80 Hz, 1H), 4.12 (d, J=17.20 Hz, 1H), 3.25-3.33 (m, 2H), 2.93 (m, 1H), 2.50-2.51 (m, 1H), 2.40-2.42 (m, 2H), 2.27-2.38 (m, 1H), 2.02-2.06 (m, 1H), 1.75 (t, J=2.00 Hz, 3H),

LCMS: 326.1 (M+H), Rt (min):1.986, Area—98.611%.

### Step-3: tert-butyl (tert-butoxycarbonyl)(4-((2-(2,6-dioxopiperidin-3-yl)-1-oxoisoindolin-4-yl)(pent-3-yn-1-yl)amino)butyl)carbamate

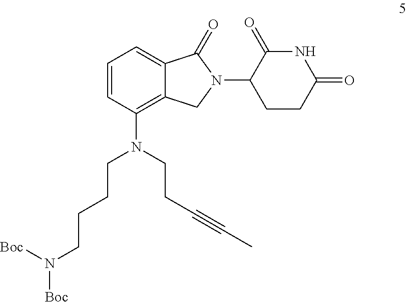 [SMILES: CC#CCCN(CCCCN(C(=O)OC(C)(C)C)C(=O)OC(C)(C)C)c1cccc2c1CN(C1CCC(=O)NC1=O)C2=O]

To a stirred mixture of 3-(1-oxo-4-(pent-3-yn-1-ylamino)isoindolin-2-yl)piperidine-2,6-dione 4 (0.5 g, 1.537 mmol, 1.0 equiv) and tert-butyl (tert-butoxycarbonyl)(5-oxopentyl)carbamate 5 (0.88 g, 3.07 mmol, 2.0 equiv) in CH₂Cl₂ (15 ml) and DMF (15 ml) was added trifluoroacetic acid (0.106 ml, 1.383 mmol, 0.9 equiv) and stirred for 10 min at 0° C. Sodium triacetoxyborohydride (0.49 g, 2.305 mmol, 1.5 equiv) was added in portions at 0° C. and the resulted colourless cloudy mass was stirred for 16 h at room temperature. Upon completion of the reaction (as confirmed by UPLC analysis), the reaction mixture was quenched with ice cold water (50 ml) and extracted with dichloromethane (2×50 ml). The combined organic layer was dried over anhy. Na₂SO₄ and concentrated under vacuum to afford the crude product as a brown liquid. The crude compound was purified by (Grace® column: C18 40 μm, 120 g; flow rate: 20 mL/min; 0.1% aqueous HCOOH/ACN mobile phase, product eluted with 70% of ACN in 0.1% HCO₂H) to afford tert-butyl (tert-butoxycarbonyl)(4-((2-(2,6-dioxopiperidin-3-yl)-1-oxoisoindolin-4-yl)(pent-3-yn-1-yl)amino)butyl)carbamate 10 (0.4 g, 0.649 mmol, 42.2% yield) as a off-white solid

¹H-NMR (400 MHz, DMSO-d6): δ 10.99 (s, 1H), 7.39 (t, J=10.00 Hz, 1H), 7.23 (d, J=9.60 Hz, 1H), 7.12 (d, J=10.80 Hz, 1H), 5.08-5.14 (m, 1H), 4.41 (d, J=22.80 Hz, 1H), 4.31 (d, J=22.40 Hz, 1H), 3.40-3.47 (m, 2H), 3.23-3.33 (m, 2H), 2.87-2.93 (m, 1H), 2.28-2.45 (m, 1H), 2.01-2.08 (m, 2H), 1.69 (t, J=2.00 Hz, 3H), 1.49 (s, 18H), 1.27-1.44 (m, 8H),

LCMS: 597.4 (M+H), Rt (min):2.482, Area—96.894%.

### Step-4: tert-butyl (Z)-(tert-butoxycarbonyl)(4-((2-(2,6-dioxopiperidin-3-yl)-1-oxoisoindolin-4-yl)(pent-3-en-1-yl)amino)butyl)carbamate

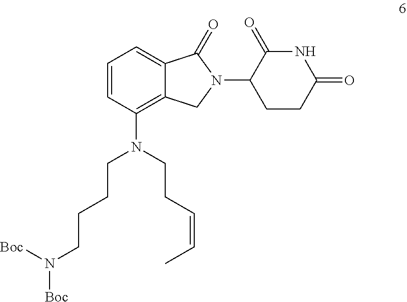 [SMILES: C/C=C\CCN(CCCCN(C(=O)OC(C)(C)C)C(=O)OC(C)(C)C)c1cccc2c1CN(C1CCC(=O)NC1=O)C2=O]

To a stirred solution of tert-butyl (tert-butoxycarbonyl)(4-((2-(2,6-dioxopiperidin-3-yl)-1-oxoisoindolin-4-yl)(pent-3-yn-1-yl)amino)butyl)carbamate 5 (150 mg, 0.251 mmol, 1.0 equiv), quinoline (39.0 mg, 0.302 mmol, 1.2 equiv) in DMF (2.0 ml) was added Lindlar catalyst (12 mg, 0.113 mmol, 0.449 equiv) and stirred under hydrogen atmosphere at room temperature for 16 h. Upon completion of the reaction (as confirmed by UPLC analysis), the reaction mixture was filtered through Celite bed and washed with DMF (2 ml), the filtrate was purified by reverse phase chromatography (Grace® column: C18 40 μm, 120 g; flow rate: 20 mL/min; 0.1% aqueous HCOOH/ACN mobile phase, product eluted at 70-75% ACN in 0.1% of HCOOH) to afford tert-butyl (Z)-(tert-butoxycarbonyl)(4-((2-(2,6-dioxopiperidin-3-yl)-1-oxoisoindolin-4-yl)(pent-3-en-1-yl)amino)butyl)carbamate 6 (120 mg, 0.182 mmol, 72.2% yield) as a pale yellow gummy solid.

¹H-NMR (400 MHz, DMSO-d6): δ 10.98 (s, 1H), 7.39 (t, J=3.20 Hz, 1H), 7.21 (d, J=4.80 Hz, 1H), 7.11 (d, J=8.00 Hz, 1H), 5.38-5.45 (m, 2H), 5.09-5.14 (m, 1H), 4.39 (d, J=16.80 Hz, 1H), 4.28 (d, J=17.20 Hz, 1H), 3.36-3.47 (m, 3H), 3.18-3.24 (m, 4H), 3.18-3.20 (m, 1H), 2.50-2.53 (m, 2H), 2.44-2.50 (m, 1H), 2.16-2.18 (m, 2H), 2.00-2.01 (m, 1H), 1.59 (d, J=4.80 Hz, 3H), 1.49 (s, 18H), 1.27-1.44 (m, 2H),

LCMS: 599.3 (M+H). Method: Column: Atlantis dC18 (50×4.6 mm) 5 μm, Mobile phase:A: 0.1% FA in H₂O, Mobile phase: B: Acetonitrile, Flow Rate: 1.5 ml/min

HPLC: Method: Mobile phase: A: 0.1% TFA in water, Mobile phase: B: Methanol, Flow:1.0 mL/min, Column: Atlantis dC18 (250×4.6) mm, 5 μm, RT: 16.959 min, Area:90.666%.

### Step-5: (Z)-3-(4-((4-aminobutyl)(pent-3-en-1-yl)amino)-1-oxoisoindolin-2-yl)piperidine-2,6-dione

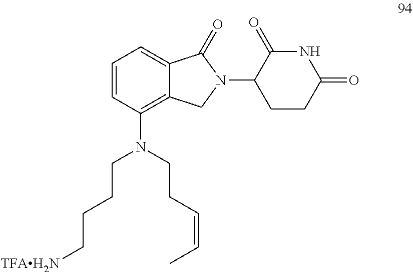

To a stirred solution of tert-butyl (Z)-(tert-butoxycarbonyl)(4-((2-(2,6-dioxopiperidin-3-yl)-1-oxoisoindolin-4-yl)(pent-3-en-1-yl)amino)butyl)carbamate 6 (60 mg, 0.100 mmol) in CH₂Cl₂ (1 ml) was added TFA (2 ml, 26.0 mmol) at 0° C. and then stirred at room temperature for overnight. Upon completion of the reaction (as confirmed by UPLC), the reaction mixture was concentrated under reduced pressure at 40° C., to afford pale yellow thick liquid. The crude compound was purified by preparative HPLC (Column: Atlantis 19×250 mm, mobile phase:0.1% TFA in Water/ACN, Flow rate: 2 mg/ml) to afford (Z)-3-(4-((4-aminobutyl)(pent-3-en-1-yl)amino)-1-oxoisoindolin-2-yl)piperidine-2,6-dione, TFA 94 (12 mg, 0.022 mmol, 22.15% yield) as pale yellow solid.

¹H-NMR (400 MHz, DMSO-d6): δ 11.00 (s, 1H), 7.61 (s, 3H), 7.41 (t, J=7.60 Hz, 1H), 7.24 (d, J=6.80 Hz, 1H), 7.14 (d, J=7.60 Hz, 1H), 5.44-5.46 (m, 1H), 5.38-5.39 (m, 1H), 5.11-5.15 (m, 1H), 4.39 (d, J=16.80 Hz, 1H), 4.28 (d, J=16.80 Hz, 1H), 3.22 (t, J=8.40 Hz, 4H), 2.79-2.80 (m, 1H), 2.68-2.77 (m, 2H), 2.63-2.68 (m, 2H), 2.50-2.51 (m, 1H), 2.33-2.34 (m, 2H), 2.17-2.19 (m, 1H), 1.50 (t, J=2.00 Hz, 3H), 1.48-1.49 (m, 3H),

LCMS: 399.2 (M+H). Method: Column: Atlantis dC18 (50×4.6 mm) 5 μm, Mobile phase: A: 0.1% FA in H₂O, Mobile phase: B: Acetonitrile, Flow Rate: 1.5 ml/min

HPLC: Method: Mobile phase: A: 0.1% TFA in water, Mobile phase: B: ACN, Flow:1.0 mL/min, Column: Atlantis dC18 (250×4.6) mm, 5 μm, RT: 7.709 min, Area:94.84%.

### Hydrochloride Salt Form of Compound 95

### 3-(4-((4-aminobutyl)(pent-3-yn-1-yl)amino)-1-oxoisoindolin-2-yl)piperidine-2,6-dione, HCl

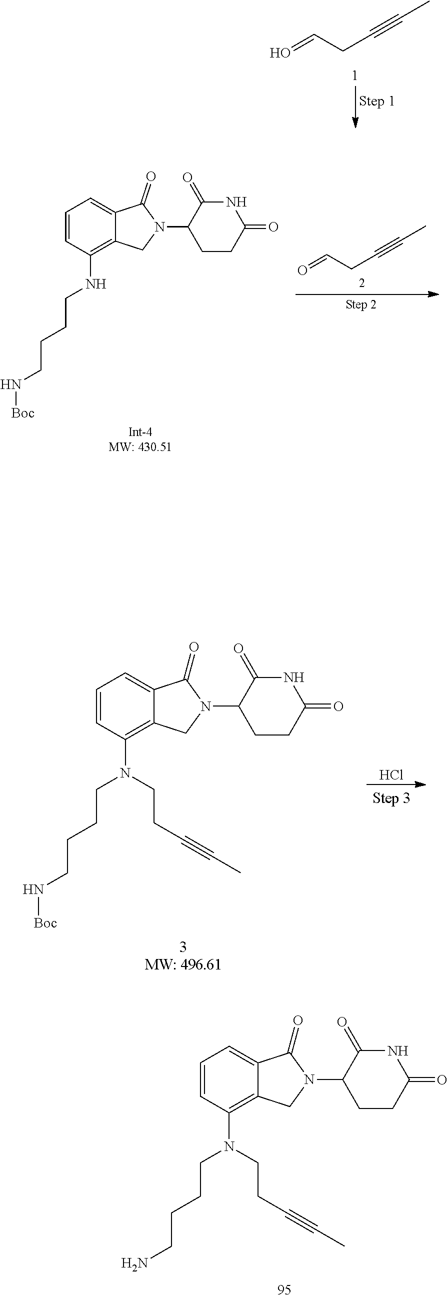

### Steps 1 & 2: tert-butyl (4-((2-(2,6-dioxopiperidin-3-yl)-1-oxoisoindolin-4-yl)(pent-3-yn-1-yl)amino)butyl)carbamate

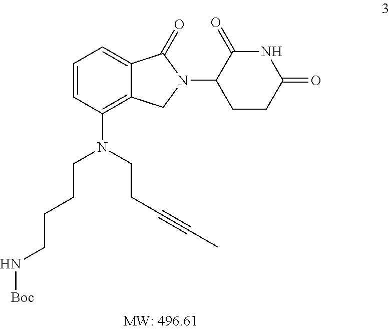 [SMILES: CC#CCCN(CCCCNC(=O)OC(C)(C)C)c1cccc2c1CN(C1CCC(=O)NC1=O)C2=O]

To a solution of pent-3-yn-1-ol 3 (2 g, 23.78 mmol, 1 equiv) in DCM (40 ml) was added Dess-Martin periodinate (11.09 g, 26.2 mmol, 1.1 equiv) at 0° C. and the resulted white suspension was stirred for 1 h at room temperature. The reaction mixture was quenched with a mixture of NaHSO₃ (5 g) in saturated NaHCO₃ (50 mL) solution and extracted with DCM (2×15 mL). To the combined organic layer was added tert-butyl (4-((2-(2,6-dioxopiperidin-3-yl)-1-oxoisoindolin-4-yl)amino)butyl)carbamate (0.500 g, 1.162 mmol, 1 equiv) and acetic acid (0.714 g, 11.627 mmol) were added and stirred for 2 h. Then NaCNBH₃ (0.463 g, 11.627 mmol, 10 equiv) was added and allowed to stir for 24 h at room temperature. Upon completion of reaction (confirmed by UPLC), the reaction mixture was quenched with water (40 ml) and extracted with DCM (2×40 mL). The combined organic layer was dried over anhy. Na₂SO₄ and concentrated under vacuum to get the crude product which was purified by Isolera (Biotage R snap cartridge, KP-Sil, 25 g, 230-400 silica mesh) using 60-70% ethyl acetate in pet ether. The pure fractions were concentrated under vacuum to afford tert-butyl (4-((2-(2,6-dioxopiperidin-3-yl)-1-oxoisoindolin-4-yl)(pent-3-yn-1-yl)amino)butyl)carbamate 3 (150 mg, 0.113 mmol, 0.474% yield) as off white solid.

LCMS: 497.2 (M+H), Rt (min): 2.384, Area %: 37.322.

### Step-3:3-(4-((4-aminobutyl)(pent-3-yn-1-yl)amino)-1-oxoisoindolin-2-yl)piperidine-2,6-dione, HCl

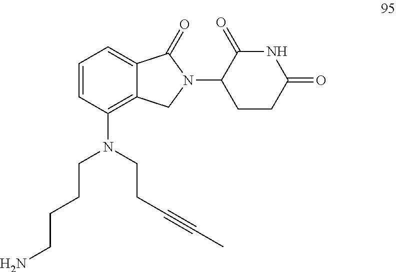 [SMILES: CC#CCCN(CCCCN)c1cccc2c1CN(C1CCC(=O)NC1=O)C2=O]

To an ice cold solution of tert-butyl (4-((2-(2,6-dioxopiperidin-3-yl)-1-oxoisoindolin-4-yl)(pent-3-yn-1-yl)amino)butyl)carbamate 3 (150 mg, 0.118 mmol, 1 equiv) in DCM (3 ml) was added HCl (4M in ethyl acetate, 0.294 mL, 1.178 mmol, 10 equiv) at 0° C. and stirred for 12 h at room temperature. Upon completion of the reaction (as confirmed by LCMS), the reaction mixture was concentrated, washed with MTBE (10 mL) to give the crude product which was purified by Prep HPLC (Column: X-Bridge-c18 19.1×250; Method: 0.1% HCL in Water/ACN; Flow rate:15 ml/min) to afford 3-(4-((4-aminobutyl)(pent-3-yn-1-yl)amino)-1-oxoisoindolin-2-yl)piperidine-2,6-dione, HCl 95 (7.5 mg, 0.017 mmol, 14.16% yield) as white solid.

¹H-NMR (400 MHz, DMSO-d6): δ 11.00 (s, 1H), 7.78 (s, 1H), 7.41 (t, J=7.60 Hz, 1H), 7.25 (d, J=7.20 Hz, 1H), 7.15 (d, J=8.00 Hz, 1H), 5.15-5.10 (m, 1H), 4.45-4.30 (m, 2H), 3.33 (t, J=7.20 Hz, 2H), 3.24 (t, J=6.40 Hz, 3H), 2.93 (s, 1H), 2.77-2.68 (m, 2H), 2.33-2.28 (m, 2H), 2.04 (t, J=5.20 Hz, 1H), 1.70 (t, J=2.40 Hz, 2H), 1.52 (d, J=4.00 Hz, 4H), 1.24 (t, J=3.20 Hz, 1H), 0.84 (t, J=7.20 Hz, 1H).

LCMS: 397.3 (M+H), Method: Mobile phase: A: 0.1% TFA in H₂O, Mobile phase: B: 0.1% TFA in ACN, Column: X Bridge C8 (50×4.6 mm) 3.5 μm, Flow Rate: 1.5 ml/min, Rt (min): 1.340, Area %: 96.271.

HPLC: Method: Mobile phase: A: 0.1% TFA in water, Mobile phase: B: ACN, Column: X-Bridge C8(50×4.6) mm, 3.5 μm, Flow: 2.0 mL/min, Rt (min): 2.314, Area %: 98.115.

### Hydrochloride Salt Form of Compound 96

### 3-(4-(bis(4-aminobutyl)amino)-1-oxoisoindolin-2-yl)piperidine-2,6-dione dihydrochloride

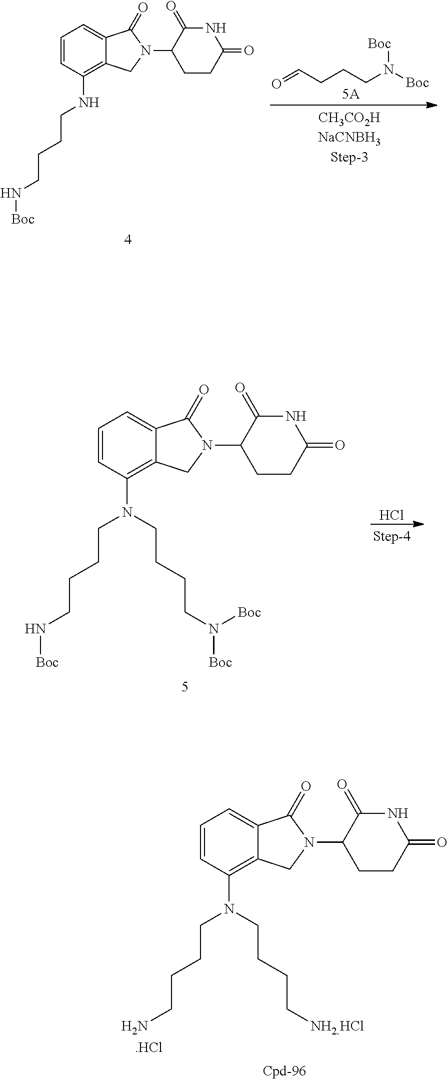

### Step-3: di-tert-butyl (((2-(2,6-dioxopiperidin-3-yl)-1-oxoisoindolin-4-yl)azanediyl)bis(butane-4,1-diyl))dicarbamate

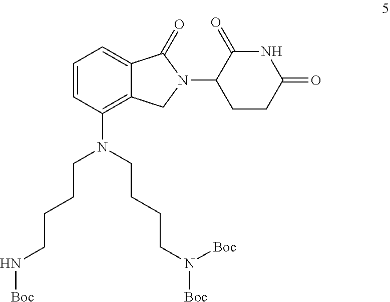 [SMILES: CC(C)(C)OC(=O)NCCCCN(CCCCN(C(=O)OC(C)(C)C)C(=O)OC(C)(C)C)c1cccc2c1CN(C1CCC(=O)NC1=O)C2=O]

To a solution of tert-butyl (4-((2-(2,6-dioxopiperidin-3-yl)-1-oxoisoindolin-4-yl)amino)butyl)carbamate 4 (250 mg, 0.581 mmol), tert-butyl (tert-butoxycarbonyl)(4-oxobutyl)carbamate 5A (334 mg, 1.161 mmol, 2 equiv) and TFA (0.179 ml, 2.323 mmol, 4 equiv) in a mixture of Dichloromethane (0.5 mL, 20 vol) and DMF (0.25 mL, 10 vol) was added sodium triacetoxyborohydride (492 mg, 2.323 mmol, 4 equiv) and stirred for 16 h at room temperature. Upon completion of the reaction (as confirmed by TLC analysis, EtOAc, R_{f}˜0.6, and UPLC), the reaction mixture was diluted with DCM (5 mL) and washed water (3×10 mL) and brine (10 mL), dried over Na₂SO₄, filtered and concentrated. The resulted residue was purified by chromatography (column size: Biotage R snap cartridge, KP-Sil, 25 g, 230-400 silica gel) using 75-80% ethyl acetate in pet ether to obtain tert-butyl (tert-butoxycarbonyl)(4-((4-((tert-butoxycarbonyl)amino)butyl)(2-(2,6-dioxopiperidin-3-yl)-1-oxoisoindolin-4-yl)amino)butyl) carbamate 5 (150 mg, 36.5% yield) as white solid.

¹H-NMR (400 MHz, DMSO-d6): δ 10.98 (s, 1H), 7.37 (t, J=7.60 Hz, 1H), 7.20 (d, J=7.20 Hz, 1H), 7.08 (d, J=8.00 Hz, 1H), 6.78 (d, J=5.20 Hz, 1H), 5.11 (q, J=5.20 Hz, 1H), 4.39 (d, J=17.20 Hz, 1H), 4.28 (d, J=16.80 Hz, 1H), 3.34 (s, 2H), 3.18 (d, J=6.80 Hz, 4H), 2.90 (s, 4H), 2.00 (s, 2H), 1.40 (s, 33H).

LC-MS: 702.4 (M+H), Rt (min): 2.83, Area %: 99.289.

### Step-4: 3-(4-(bis(4-aminobutyl)amino)-1-oxoisoindolin-2-yl)piperidine-2,6-dione dihydrochloride

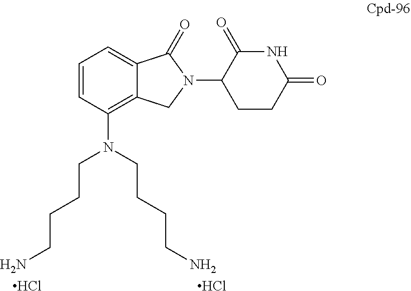 [SMILES: NCCCCN(CCCCN)c1cccc2c1CN(C1CCC(=O)NC1=O)C2=O ; Cl ; Cl]

To an ice cold solution of di-tert-butyl (((2-(2,6-dioxopiperidin-3-yl)-1-oxoisoindolin-4-yl)azanediyl)bis(butane-4,1-diyl))dicarbamate 5 (150 mg, 0.249 mmol) in DCM (5 mL) was added HCl (4M soln. in dioxane) (3 ml, 12.00 mmol) and stirred for 2 h at room temperature. Upon completion of the reaction (as confirmed by TLC analysis, 100% EtOAc), the reaction mixture was concentrated and lyophilized to afford 3-(4-(bis(4-aminobutyl)amino)-1-oxoisoindolin-2-yl)piperidine-2,6-dione, 2HCl 96 (82 mg, 67.8% yield) (2.4 g, 98%) as pale-yellow solid.

¹H-NMR (400 MHz, DMSO-d6): δ 11.02 (s, 1H), 8.07 (s, 6H), 7.52 (s, 3H), 5.12 (q, J=5.20 Hz, 1H), 4.65 (s, 8H), 3.42-3.43 (m 1H), 2.74 (d, J=5.20 Hz, 4H), 2.62 (d, J=16.80 Hz, 1H), 2.07 (t, J=5.60 Hz, 1H), 1.56 (d, J=4.80 Hz, 8H).

¹H-NMR (400 MHz, CD3OD): δ 7.93 (s, 2H), 7.80 (s, 1H), 5.24 (q, J=4.80 Hz, 1H), 4.48 (q, J=400.80 Hz, 2H), 3.66 (t, J=26.40 Hz, 4H), 3.00-2.92 (m, 6H), 2.68-2.61 (m, 1H), 2.28-2.24 (m, 1H), 1.69 (s, 1H).

LCMS: 402.3 (M+H). Method: Mobile Phase A: 0.1% TFA in H₂O. Mobile Phase B: 0.1% TFA in ACN. Flow rate: 1.5 ml/min. Column: XBridge C8 (50×4.6 mm) 3.5 μm. Rt (min): 0.887; Area %: 97.781.

HPLC: Method: Mobile Phase A: 0.1% TFA in H₂O. Mobile Phase B: Acetonitrile. Flow rate: 2.0 mL/min. Column: X-Bridge C8(50×4.6) mm, 3.5 μm. Rt (min): 1.08; Area %: 95.532.

### Hydrochloride Salt Form of Compound 97

### 3-(4-((4-aminobutyl)(3-aminopropyl)amino)-1-oxoisoindolin-2-yl)piperidine-2,6-dione dihydrochloride

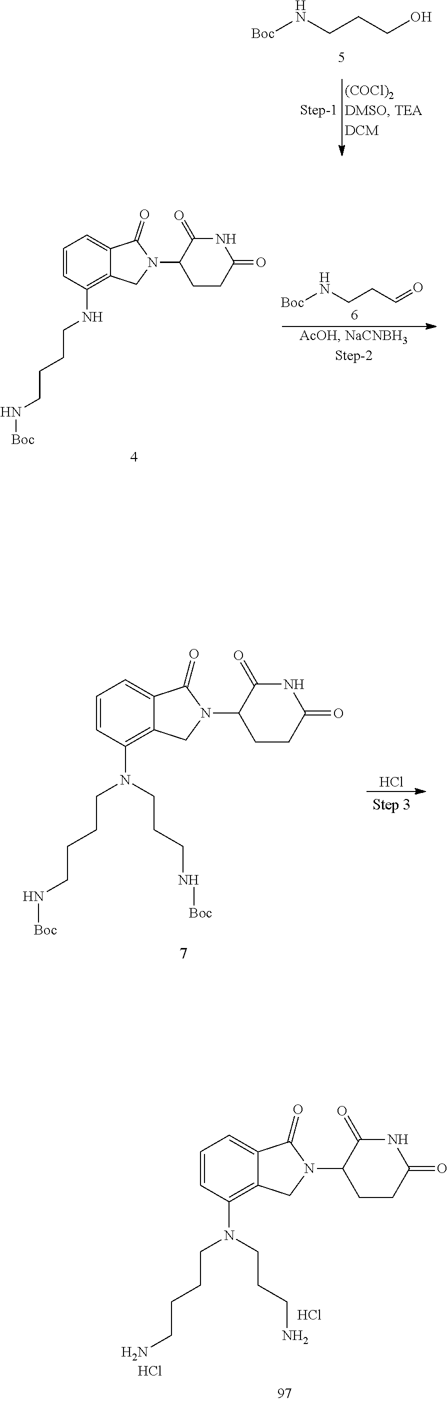

### Step-1: tert-butyl (3-oxopropyl)carbamate

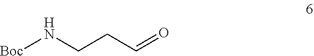 [SMILES: CC(C)(C)OC(=O)NCCC=O]

A solution of oxalyl chloride (0.599 ml, 6.85 mmol, 1.2 equiv) in DCM (10 mL) was added dropwise to a solution of DMSO (0.972 ml, 13.70 mmol, 2.4 equiv) in DCM (5 mL) at −78° C. over a period of 15 minutes under nitrogen atmosphere and stirred for 15 minutes at the same temperature. To this a solution of tert-butyl (3-hydroxypropyl)carbamate 5 (1 g, 5.71 mmol, 1 equiv) in DCM (5 mL) was added dropwise over a period of 15 minutes. After stirring the reaction mixture for 30 minutes at −78° C., was added TEA (3.98 ml, 28.5 mmol, 5.0 equiv) in drops at −78° C. Once addition was complete, the reaction mixture was slowly warmed to 0° C. and stirred for 30 minutes. Upon completion of the reaction (as confirmed by TLC analysis, 3:7/EtOAc: pet ether, R_{f}˜0.4, KMnO₄), the reaction mixture was diluted with DCM (10 mL) and washed with 10% aq citric acid solution (1×10 mL), water (1×10 mL) and brine (1×10 mL), dried over anhydrous Na₂SO₄, filtered and concentrated under reduced pressure below 30° C. to afford crude tert-butyl (3-oxopropyl)carbamate 6 (0.950 g, 4.90 mmol, 86% yield) as pale yellow liquid which was used further without purification.

LCMS: (mass peak not observed). Method: Column: Atlantis dC18 (50×4.6 mm) 5 μm Mobile phase: A: 0.1% FA in H₂O B: ACN, Flow Rate: 1.5 mL/min. Rt (min): 2.644; Area %—89.26.

### Step-2: tert-butyl (4-((3-((tert-butoxycarbonyl)amino)propyl)(2-(2,6-dioxopiperidin-3-yl)-1-oxoisoindolin-4-yl)amino)butyl)carbamate

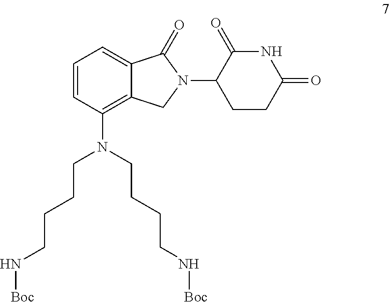 [SMILES: CC(C)(C)OC(=O)NCCCCN(CCCCNC(=O)OC(C)(C)C)c1cccc2c1CN(C1CCC(=O)NC1=O)C2=O]

tert-butyl (3-oxopropyl)carbamate 6 (0.805 g, 4.65 mmol, 4 equiv) and of tert-butyl (4-((2-(2,6-dioxopiperidin-3-yl)-1-oxoisoindolin-4-yl)amino)butyl)carbamate 4 (0.5 g, 1.161 mmol, 1 equiv) were taken together in a mixture of 1,2-Dichloroethane (10 mL, 10 vol) and DMF (5 mL, 5 vol) under nitrogen atmosphere. To this, acetic acid (0.332 ml, 5.81 mmol, 5 equiv) was added and the reaction mixture was stirred for 1 hour at room temperature. NaCNBH₃ (0.292 g, 4.65 mmol, 4 equiv) was added in portions and the reaction mixture was stirred for additional 16 hours at room temperature. Upon completion of the reaction (as confirmed by TLC analysis, 100% EtOAc Rf˜0.5, and LCMS), the reaction mixture was diluted with ice-cold water (15 mL), extracted with DCM (2×15 mL), washed with brine (25 mL), dried over anhydrous Na₂SO₄, filtered and concentrated to afford crude product as white solid, which was purified by Prep-HPLC (Method: Instrument: SC-DC-ARD-05-045; column no: XBridge C8—250-X-Select-C18-19×150 mm; 0.1% TFA in Water/ACN; Rt: 9.6 min) and lyophilized to obtain tert-butyl (4-((3-((tert-butoxycarbonyl)amino)propyl)(2-(2,6-dioxopiperidin-3-yl)-1-oxoisoindolin-4-yl)amino)butyl)carbamate 7 (60 mg, 0.092 mmol, 7.89% yield) as white solid.

LCMS: 588.4 (M+H). Method: Column: XBridge C8(50×4.6 mm) 3.5 μm Mobile phase: A: 0.1% TFA in H₂O B: 0.1% TFA in ACN, Flow Rate: 1.5 mL/min. Rt (min): 1.911; Area %—89.75.

¹H-NMR (400 MHz, DMSO-d₆): δ 10.97 (s, 1H), 7.37 (t, J=7.60 Hz, 1H), 7.14 (d, J=7.20 Hz, 1H), 7.08 (d, J=8.00 Hz, 1H), 6.82-6.76 (m, 2H), 5.10 (dd, J=5.20, 13.20 Hz, 1H), 4.35 (q, J=17.20 Hz, 2H), 3.20-3.16 (m, 4H), 2.96-2.89 (m, 5H), 2.04-2.01 (m, 1H), 1.58-1.51 (m, 2H), 1.58-1.51 (m, 23H).

### Step-3: 3-(4-((4-aminobutyl)(3-aminopropyl)amino)-1-oxoisoindolin-2-yl)piperidine-2,6-dione dihydrochloride

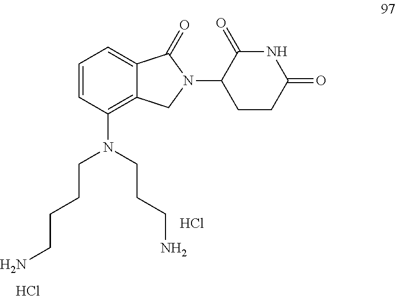 [SMILES: NCCCCN(CCCN)c1cccc2c1CN(C1CCC(=O)NC1=O)C2=O ; Cl ; Cl]

To an ice cold solution of tert-butyl (4-((3-((tert-butoxycarbonyl)amino)propyl)(2-(2,6-dioxopiperidin-3-yl)-1-oxoisoindolin-4-yl)amino)butyl)carbamate 7 (60 mg, 0.102 mmol, 1 equiv) in ethyl acetate (2 mL) was added HCl (4M soln. in EtOAc, 4 mL) and stirred for 1.5 h at room temperature. The progress of the reaction was monitored by LCMS. The reaction mixture was concentrated, washed with MTBE (2×5 mL) and lyophilized to afford 3-(4-((4-aminobutyl)(3-aminopropyl)amino)-1-oxoisoindolin-2-yl)piperidine-2,6-dione 97 (20 mg, 0.051 mmol, 50.1% yield) as off white solid.

LCMS: 388.3 (M-2×HCl). Method: Column: Atlantis dC18 (50×4.6 mm) 5 μm Mobile phase: A: 0.1% FA in H₂O B: ACN, Flow Rate:0.6 mL/min. Rt (min): 2.218; Area % —99.01.

HPLC: 93.99%, Rt (min): 6.421. Method: Column: Atlantis dC18 (250×4.6) mm, 5 μm, Mobile Phase A: 0.1% TFA in H₂O, Mobile Phase B: Methanol, Flow rate:1.0 mL/min.

¹H-NMR (400 MHz, DMSO-d₆): δ 11.00 (s, 1H), 7.94 (br s, 6H), 7.43 (t, J=7.60 Hz, 1H), 7.29-7.21 (m, 2H), 5.13 (dd, J=4.80, 13.20 Hz, 1H), 4.45 (d, J=17.20 Hz, 1H), 4.31 (d, J=16.80 Hz, 1H), 3.76-3.20 (m, 4H), 2.98-2.89 (m, 1H), 2.81-2.76 (m, 4H), 2.59 (s, 2H), 2.08-2.02 (m, 1H), 1.76-1.72 (m, 2H), 1.53 (s, 4H).

### Hydrochloride Salt Form of Compound 102

### 3-(4-((4-aminobutyl)((1S,4S)-4-aminocyclohexyl)amino)-1-oxoisoindolin-2-yl)piperidine-2,6-dione dihydrochloride

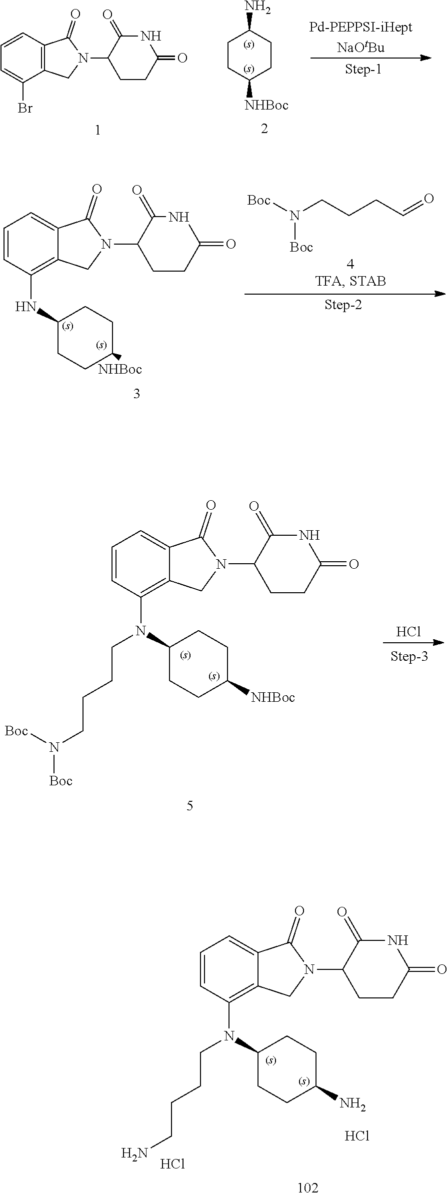

### Step-1: tert-butyl ((1S,4S)-4-((2-(2,6-dioxopiperidin-3-yl)-1-oxoisoindolin-4-yl)amino)cyclohexyl)carbamate

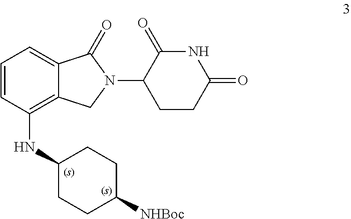

3-(4-bromo-1-oxoisoindolin-2-yl)piperidine-2,6-dione 1 (0.5 g, 1.547 mmol, 1 equiv), tert-butyl ((1S,4S)-4-aminocyclohexyl)carbamate 2 (0.995 g, 4.64 mmol, 3 equiv) and NaOt-Bu (0.446 g, 4.64 mmol, 3 equiv) were dissolved in DMF (2 ml) and de-gassed for 20 min with nitrogen. Pd-PEPPSI-IHept(Cl) (0.075 g, 0.077 mmol, 0.05 equiv) was added under nitrogen atmosphere and heated the reaction at 110° C. for 4 h. Upon completion of the reaction (as confirmed by TLC analysis, 100% EtOAc R_{f}˜0.4, and LCMS), the reaction mixture was filtered through Celite bed, washed with THF (10 mL), concentrated to minimum volume (3 mL) and purified by reverse-phase column chromatography (Grace® column: C18 40 μm, 120 g; flow rate: 20 mL/min; 0.1% aqueous HCOOH/ACN mobile phase) to obtain tert-butyl ((1S,4S)-4-((2-(2,6-dioxopiperidin-3-yl)-1-oxoisoindolin-4-yl)amino)cyclohexyl)carbamate 3 (138 mg, 0.289 mmol, 18% yield) as pale yellow solid.

LCMS: 456.0 (M+H). Method: Column: Atlantis dC18 (50×4.6 mm) 5 μm Mobile phase: A: 0.1% FA in H₂O B: ACN, Flow Rate: 1.5 mL/min. Rt (min): 2.223; Area % —95.48.

¹H-NMR (400 MHz, DMSO-d6): δ 11.04 (s, 1H), 7.28 (t, J=7.60 Hz, 1H), 6.79 (d, J=8.00 Hz, 1H), 6.63 (br s, 1H), 5.17-5.09 (m, 2H), 4.21 (q, J=17.20 Hz, 2H), 3.47-3.42 (m, 2H), 2.98-2.90 (m, 1H), 2.68-2.65 (m, 1H), 2.08-2.03 (m, 1H), 1.66 (m, 6H), 0.54 (m, 3H), 1.39 (m, 10H).

### Step-2: tert-butyl (tert-butoxycarbonyl)(4-(((1S,4S)-4-((tert-butoxycarbonyl)amino)cyclohexyl)(2-(2,6-dioxopiperidin-3-yl)-1-oxoisoindolin-4-yl)amino)butyl)carbamate

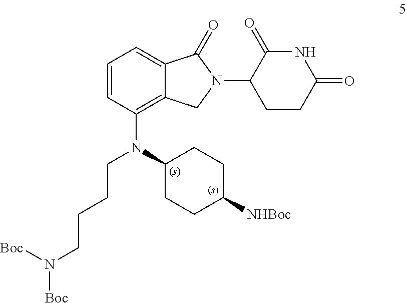 [SMILES: CC(C)(C)OC(=O)N[C@H]1CC[C@@H](N(CCCCN(C(=O)OC(C)(C)C)C(=O)OC(C)(C)C)c2cccc3c2CN(C2CCC(=O)NC2=O)C3=O)CC1]

To a solution of tert-butyl ((1S,4S)-4-((2-(2,6-dioxopiperidin-3-yl)-1-oxoisoindolin-4-yl)amino) cyclohexyl)carbamate 3 (130 mg, 0.285 mmol, 1 equiv), tert-butyl (tert-butoxycarbonyl)(4-oxobutyl)carbamate 4 (0.164 g, 0.569 mmol, 2 equiv) in DCM (4 ml) and DMF (4 ml) at 0° C. were added TFA (0.074 ml, 1.139 mmol, 4 equiv) followed by sodium triacetoxyborohydride (0.241 g, 1.139 mmol, 4 equiv). The reaction mixture was warmed to RT and stirred at the same temperature for 16 h. Upon completion of the reaction (as confirmed by TLC analysis, 100% EtOAc R_{f}˜0.5, and LCMS), the reaction mixture was diluted with ice-cold water (15 mL), extracted with ethyl acetate (2×15 mL), washed with brine (25 mL), dried over anhydrous Na₂SO₄, filtered and concentrated. The resulted residue was purified by reverse-phase column chromatography (Grace® column: C18 40 μm, 40 g; flow rate: 20 mL/min; 0.1% aqueous HCOOH/ACN mobile phase). The pure fractions from the column were lyophilized to afford tert-butyl (tert-butoxycarbonyl)(4-(((1S,4S)-4-((tert-butoxycarbonyl)amino)cyclohexyl)(2-(2,6-dioxopiperidin-3-yl)-1-oxoisoindolin-4-yl)amino)butyl)carbamate 5 (74 mg, 0.140 mmol, 58% yield) as white solid.

LCMS: 726.7 (M-H). Method: Column: Atlantis dC18 (50×4.6 mm) 5 μm Mobile phase: A: 0.1% FA in H₂O B: ACN, Flow Rate: 1.5 mL/min. Rt (min): 2.912; Area %—99.75.

¹H-NMR (400 MHz, DMSO-d6): δ 10.98 (s, 1H), 7.40 (t, J=7.60 Hz, 1H), 7.26 (dd, J=8.00, 16.60 Hz, 2H), 6.97 (d, J=7.60 Hz, 1H), 6.97 (q, J=7.60 Hz, 1H), 4.30 (q, J=17.20 Hz, 2H), 3.56 (m, 1H), 3.36-3.16 (m, 2H), 3.08 (m, 1H), 2.91-2.88 (m, 1H), 2.01-1.99 (m, 1H), 1.785 1.809 (m, 2H), 1.65-1.62 (m, 3H), 1.53 (m, 8H), 1.44-1.40 (m, 27H), 1.24-1.21 (m, 3H).

### Step-3:3-(4-((4-aminobutyl)((1S,4S)-4-aminocyclohexyl)amino)-1-oxoisoindolin-2-yl)piperidine-2,6-dione dihydrochloride

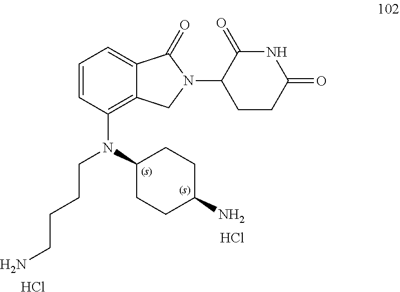 [SMILES: NCCCCN(c1cccc2c1CN(C1CCC(=O)NC1=O)C2=O)[C@H]1CC[C@@H](N)CC1 ; Cl ; Cl]

To an ice cold solution of tert-butyl (tert-butoxycarbonyl)(4-(((1S,4S)-4-((tert-butoxycarbonyl)amino)cyclohexyl)(2-(2,6-dioxopiperidin-3-yl)-1-oxoisoindolin-4-yl)amino)butyl)carbamate 5 (72 mg, 0.099 mmol, 1 equiv) in DCM (1 mL) was added HCl (4M soln. in EtOAc, 2.5 mL) and stirred for 2 h at room temperature. The progress of the reaction was monitored by LCMS. The reaction mixture was concentrated, washed with MTBE (2×5 mL) and lyophilized to afford 3-(4-((4-aminobutyl)((1S,4S)-4-aminocyclohexyl)amino)-1-oxoisoindolin-2-yl)piperidine-2,6-dione dihydrochloride 102 (49 mg, 0.095 mmol, 96% yield) as off white solid.

LCMS: 427.2 (M-HCl). Method: Column: XBridge C8 (50×4.6 mm) 3.5 μm Mobile phase: A: 0.1% TFA in H₂O B: 0.1% TFA in ACN, Flow Rate: 1.5 mL/min. Rt (min): 0.932; Area %—94.26.

HPLC: 95.86%, Rt (min): 1.363. Method: Column: X-Bridge C8(50×4.6) mm, 3.5p m, Mobile phase: A: 0.1% TFA in water, Mobile phase: B: ACN, Flow: 2.0 mL/min.

¹H-NMR (400 MHz, DMSO-d₆): δ 10.99 (s, 1H), 8.23 (s, 3H), 7.92 (s, 3H), 7.44 (d, J=7.20 Hz, 3H), 5.10 (dd, J=5.20, 12.80 Hz, 1H), 4.47-4.29 (m, 2H), 3.25 (s, 4H), 2.73-2.70 (m, 1H), 2.68-2.68 (m, 4H), 2.05-2.02 (m, 1H), 1.92-1.83 (m, 4H), 1.92-1.83 (m, 6H), 1.32 (s, 2H).

### Hydrochloride Salt Form of Compound 103

### 3-(4-((4-aminobutyl)((1r,4r)-4-aminocyclohexyl)amino)-1-oxoisoindolin-2-yl)piperidine-2,6-dione, 2HCl

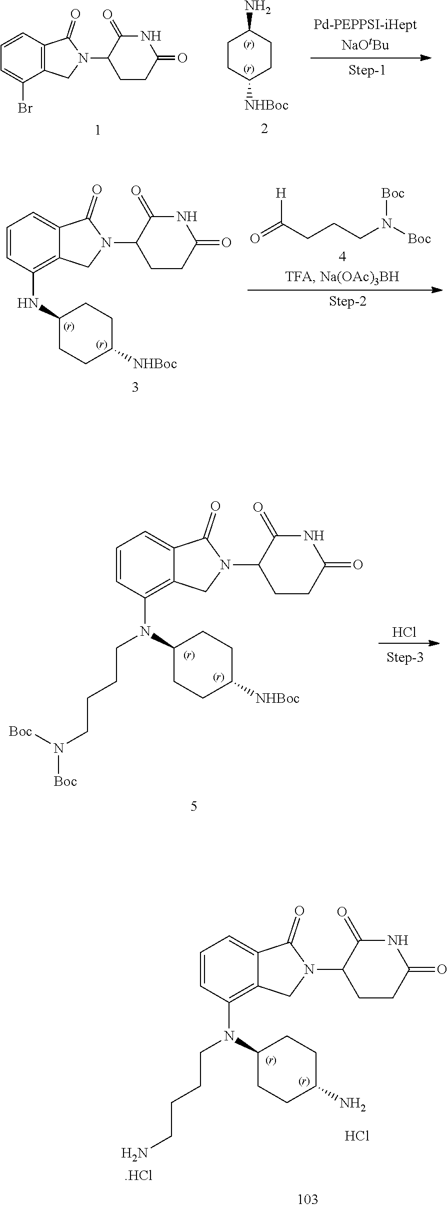

### Step-1: tert-butyl ((1R,4R)-4-((2-(2,6-dioxopiperidin-3-yl)-1-oxoisoindolin-4-yl)amino)cyclohexyl)carbamate

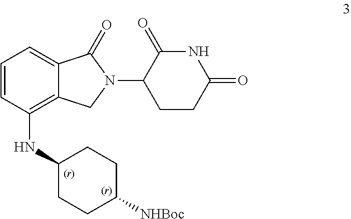

3-(4-bromo-1-oxoisoindolin-2-yl)piperidine-2,6-dione 1 (0.5 g, 1.547 mmol, 1 equiv), tert-butyl ((1R,4R)-4-aminocyclohexyl)carbamate 2 (0.995 g, 4.64 mmol, 3 equiv) and NaOt-Bu (0.446 g, 4.64 mmol, 3 equiv) were dissolved in DMF (2 ml) and de-gassed for 20 min with nitrogen. Pd-PEPPSI-IHept(Cl) (0.075 g, 0.077 mmol, 0.05 equiv) was added under nitrogen atmosphere and heated the reaction at 110° C. for 4 h. Upon completion of the reaction (as confirmed by UPLC analysis, the reaction mixture was filtered through celite, washed with THF (10 mL), concentrated to minimum volume (3 mL) and purified by reverse-phase column chromatography (Grace® column: C18 40 μm, 100 g; flow rate: 20 mL/min; 0.1% aqueous HCOOH/ACN mobile phase) to obtain tert-butyl ((1R,4S)-4-((2-(2,6-dioxopiperidin-3-yl)-1-oxoisoindolin-4-yl)amino)cyclohexyl)carbamate 3 (0.070 g, 0.152 mmol, 9.81% yield) as cream colour solid.

LCMS: 401.1 (M-Boc). Method: Column: Atlantis dC18 (50×4.6 mm) 5 μm Mobile phase: A: 0.1% FA in H₂O B: ACN, Flow Rate: 1.5 mL/min. Rt (min): 2.200, Area %—99.01

¹H-NMR (400 MHz, DMSO-d6): δ 11.03 (s, 1H), 7.27 (t, J=7.20 Hz, 1H), 6.91 (d, J=7.20 Hz, 1H), 6.80 (t, J=8.00 Hz, 2H), 5.29 (d, J=6.80 Hz, 1H), 5.12 (q, J=4.80 Hz, 1H), 4.16 (q, J=17.20 Hz, 2H), 2.93-2.89 (m, 1H), 2.70-2.60 (m, 2H), 2.30-2.20 (m, 2H), 2.09-1.99 (m, 3H), 1.80 (m, 2H), 1.39 (s, 9H), 1.32-1.24 (m, 4H).

### Step-2: tert-butyl (tert-butoxycarbonyl)(4-(((1R,4R)-4-((tert-butoxycarbonyl)amino)cyclohexyl)(2-(2,6-dioxopiperidin-3-yl)-1-oxoisoindolin-4-yl)amino)butyl)carbamate

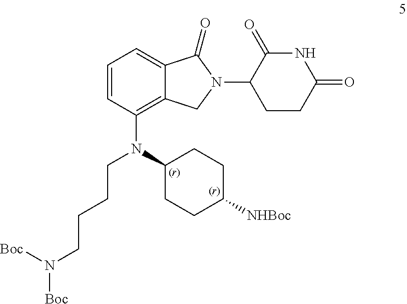 [SMILES: CC(C)(C)OC(=O)N[C@H]1CC[C@H](N(CCCCN(C(=O)OC(C)(C)C)C(=O)OC(C)(C)C)c2cccc3c2CN(C2CCC(=O)NC2=O)C3=O)CC1]

To a solution of tert-butyl ((1R,4R)-4-((2-(2,6-dioxopiperidin-3-yl)-1-oxoisoindolin-4-yl)amino) cyclohexyl)carbamate 3 (70 mg, 0.153 mmol, 1 equiv), tert-butyl (tert-butoxycarbonyl)(4-oxobutyl)carbamate 4 (88 mg, 0.307 mmol, 2 equiv) in DCM (5 ml) and DMF (0.5 ml) at 0° C. was added TFA (0.047 ml, 0.613 mmol, 4 equiv) followed by sodium triacetoxyborohydride (130 mg, 0.613 mmol, 4 equiv). The reaction mixture was stirred at room temperature for 16 h. Upon completion of the reaction (as confirmed by TLC analysis, 100% EtOAc R_{f}˜0.5, and LCMS), the reaction mixture was diluted with ice-cold water (15 mL), extracted with DCM (2×10 mL), washed with brine (25 mL), dried over Na₂SO₄, filtered and concentrated. The resulted residue was purified by reverse-phase column chromatography (Grace® column: C18 40 μm, 100 g; flow rate: 20 mL/min; 0.1% aqueous HCOOH/ACN mobile phase). The pure fractions from the column were lyophilized to afford tert-butyl (tert-butoxycarbonyl)(4-(((1R,4R)-4-((tert-butoxycarbonyl)amino)cyclohexyl)(2-(2,6-dioxopiperidin-3-yl)-1-oxoisoindolin-4-yl)amino)butyl)carbamate 5 (40 mg, 0.055 mmol, 35.8% yield) as white solid.

LCMS: 728.5 (M+H). Method: Column: Atlantis dC18 (50×4.6 mm) 5 μm Mobile phase: A: 0.1% FA in H₂O B: ACN, Flow Rate: 1.5 mL/min. Rt (min): 2.913; Area %—99.75.

### Step-3: 3-(4-((4-aminobutyl)((1R,4R)-4-aminocyclohexyl)amino)-1-oxoisoindolin-2-yl)piperidine-2,6-dione, 2HCl

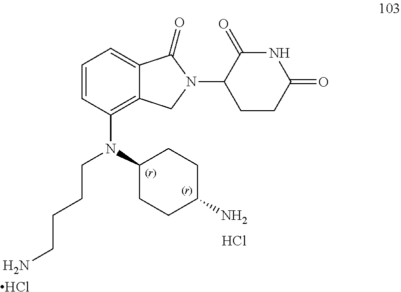 [SMILES: NCCCCN(c1cccc2c1CN(C1CCC(=O)NC1=O)C2=O)[C@H]1CC[C@H](N)CC1 ; Cl ; Cl]

To an ice cold solution of tert-butyl (tert-butoxycarbonyl)(4-(((1r,4r)-4-((tert-butoxycarbonyl)amino)cyclohexyl)(2-(2,6-dioxopiperidin-3-yl)-1-oxoisoindolin-4-yl)amino)butyl)carbamate 5 (40 mg, 0.055 mmol, 1 equiv) in DCM (1 mL) was added HCl (4M soln. in EtOAc, 1 mL, 4.0 mmol) and stirred for 2 h at room temperature. Upon completion of the reaction (as confirmed by TLC analysis, 10% MeOH/DCM, R_{f}˜0.1), the reaction mixture was concentrated, washed with MTBE (2×5 mL) and further lyophilized to afford 3-(4-((4-aminobutyl)((1r,4r)-4-aminocyclohexyl)amino)-1-oxoisoindolin-2-yl)piperidine-2,6-dione, 2HCl 103 (13.2 mg, 0.026 mmol, 47.8% yield) as off white solid.

LCMS: 428.3 (M+H) Method: Column: XBridge C8 (50×4.6 mm) 3.5 μm Mobile phase: A: 0.1% TFA in H₂O Mobile phase: B: 0.1% TFA in ACN, Flow Rate: 1.5 ml/min, Rt (min): 0.906; Area %—95.74.

HPLC: 99.63%, Rt (min): 11.860. Method: Column: Atlantis dC18(250×4.6) mm, 5 μm, Mobile phase A: 0.1% TFA in water, Mobile phase B: Methanol, Flow rate: 0.7 ml/min

¹H-NMR (400 MHz, DMSO-d₆): δ 10.98 (s, 1H), 7.96 (s, 3H), 7.80 (s, 3H), 7.44 (d, J=7.20 Hz, 1H), 7.34 (t, J=8.80 Hz, 2H), 5.12 (q, J=5.20 Hz, 1H), 4.38-4.23 (m, 2H), 3.13 (s, 2H), 2.98-2.89 (m, 3H), 2.68 (q, J=1.60 Hz, 2H), 2.61 (d, J=18.00 Hz, 2H), 2.04-1.91 (m, 3H), 1.78 (s, 2H), 1.62-1.50 (m, 4H), 1.45 (t, J=25.60 Hz, 4H).

### Hydrochloride Salt Form of Compound 104

### 3-(4-((4-aminobutyl)((1s,4s)-4-(aminomethyl)cyclohexyl)amino)-1-oxoisoindolin-2-yl)piperidine-2,6-dione dihydrochloride

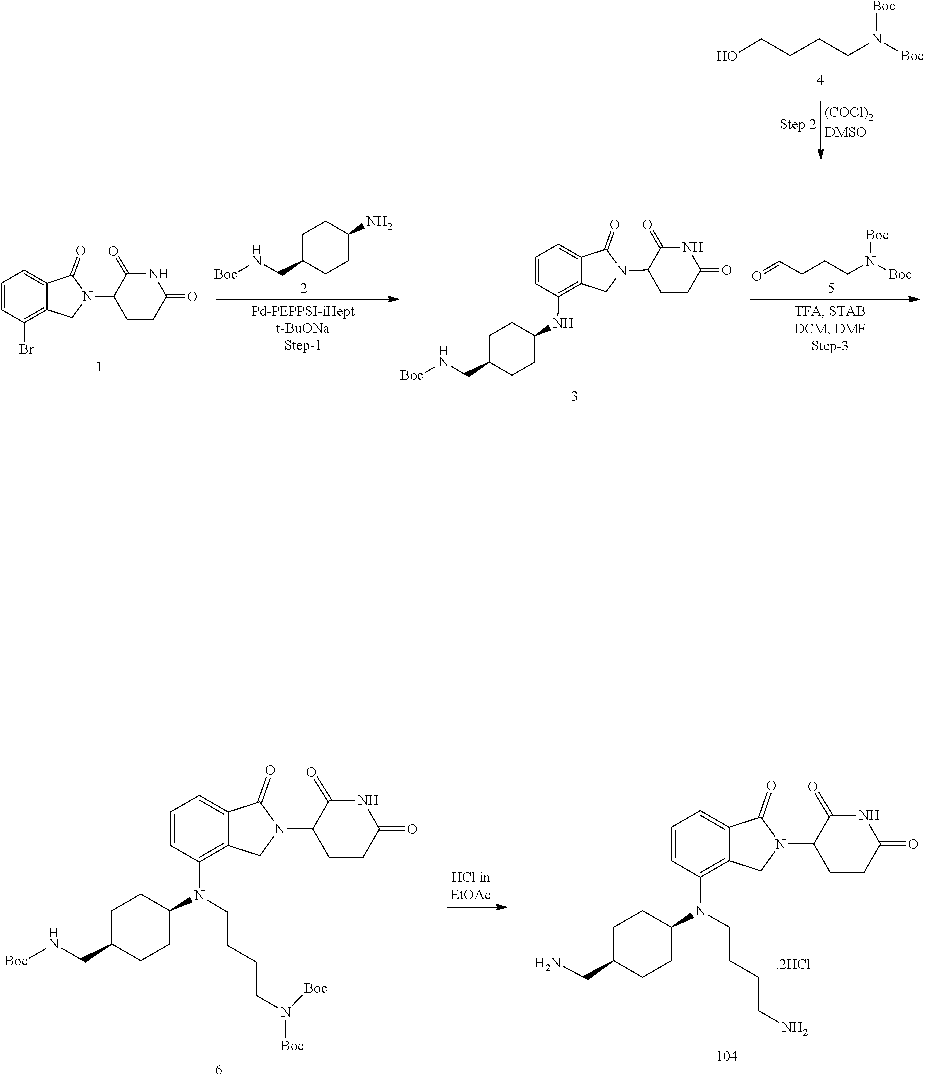

### Step-1:tert-butyl(((1s,4s)-4-((2-(2,6-dioxopiperidin-3-yl)-1-oxoisoindolin-4-yl)amino) cyclohexyl)methyl)carbamate

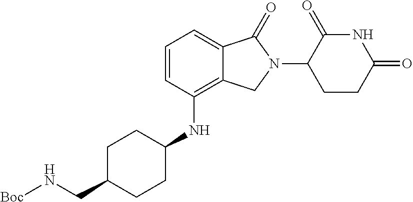 [SMILES: CC(C)(C)OC(=O)NC[C@H]1CC[C@@H](Nc2cccc3c2CN(C2CCC(=O)NC2=O)C3=O)CC1]

To a solution of 3-(4-bromo-1-oxoisoindolin-2-yl)piperidine-2,6-dione 1 (1.0 g, 3.09 mmol, 1.0 equiv) in DMF (5 ml, 5 vol), tert-butyl(((1s,4s)-4-aminocyclohexyl) methyl)carbamate 2 (1.060 g, 4.64 mmol, 1.5 equiv) and sodium tert-butoxide (0.892 g, 9.28 mmol, 3.0 equiv) were added at room temperature and degassed by bubbling with nitrogen gas for a period of 15 min. To this reaction mixture, Pd-PEPPSI-IHept(Cl) (0.151 g, 0.155 mmol), 0.05 equiv) was added under nitrogen atmosphere, degassed for 5 min and the mixture was stirred at 85° C. for overnight. Upon completion of the reaction (as confirmed by TLC and UPLC analysis, 100% EtOAc, R_{f} 0.5). The reaction mixture was filtered through Celite bed and washed with THF (50 ml), the filtrate was concentrated under reduced pressure to afford brown thick liquid. The crude compound was purified by reverse phase chromatography (product eluted with 75% of ACN in 0.1% HCO₂H). The pure fraction was lyophilized to afford tert-butyl (((1s,4s)-4-((2-(2,6-dioxopiperidin-3-yl)-1-oxoisoindolin-4-yl)amino)cyclohexyl)methyl) carbamate 3 (0.2 g, 0.350 mmol, 11.30% yield) as a pale yellow solid.

¹H-NMR (400 MHz, DMSO-d₆): δ 11.03 (s, 1H), 7.25-7.29 (m, 1H), 6.90-6.93 (m, 1H), 6.80-6.87 (m, 1H), 6.77-6.79 (m, 1H), 5.11-5.21 (m, 2H), 4.15-4.30 (m, 2H), 3.47-3.51 (m, 1H), 2.92-2.99 (m, 3H), 2.67-2.68 (m, 1H), 2.30-2.34 (m, 1H), 2.02-2.08 (m, 1H), 1.41-1.46 (m, 3H), 1.38 (m, 15H).

LCMS: 415.8 (M-tBu), Rt (min):2.324, Area—89.8%.

HPLC: Rt (min): 5.496; Area—82.3%.

### Step-2: tert-butyl (tert-butoxycarbonyl)(4-oxobutyl)carbamate

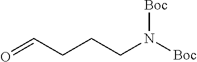 [SMILES: CC(C)(C)OC(=O)N(CCCC=O)C(=O)OC(C)(C)C]

A solution of oxalyl chloride (1.815 ml, 20.73 mmol, 2.0 equiv) in DCM (20 mL) was added dropwise to a solution of DMSO (2.94 ml, 41.5 mmol, 4.0 equiv) in DCM (5 mL) at −78° C. over a period of 15 min under nitrogen atmosphere and stirred for 15 min at the same temperature. To this, a solution of tert-butyl (tert-butoxycarbonyl)(4-hydroxybutyl)carbamate 4 (3.0 g, 10.37 mmol) in CH₂Cl₂ (5 ml) was added dropwise over a period of 15 min. After stirring the reaction mixture for 30 minutes at −78° C., triethylamine (8.67 ml, 62.2 mmol, 6.0 equiv) was added in drops at −78° C. Then, the reaction mixture was slowly warmed to 0° C. and stirred for 30 min. Upon completion of the reaction (as confirmed by TLC analysis, 20% EtOAc/pet ether, R_{f}˜0.6, KMnO₄), the reaction mixture was diluted with DCM (40 mL) and washed with 10% aq citric acid solution (1×40 mL), and water (1×40 mL), dried over Na₂SO₄, filtered and concentrated under vacuum (below 30° C.) to afford of tert-butyl (tert-butoxycarbonyl)(4-oxobutyl)carbamate 5 (2.9 g, 9.99 mmol, crude) as pale yellow liquid which was used further without purification.

¹H-NMR (400 MHz, DMSO-d6): δ 9.66 (s, 1H), 3.47-3.50 (m, 1H), 2.51-2.55 (m, 2H), 2.42-2.44 (m, 2H), 1.69-1.75 (m, 2H), 1.43-1.45 (s, 18H),

### Step-3: tert-butyl (tert-butoxycarbonyl)(4-(((1s,4s)-4-(((tert-butoxycarbonyl) amino) methyl)cyclohexyl)(2-(2,6-dioxopiperidin-3-yl)-1-oxoisoindolin-4-yl)amino)butyl)carbamate

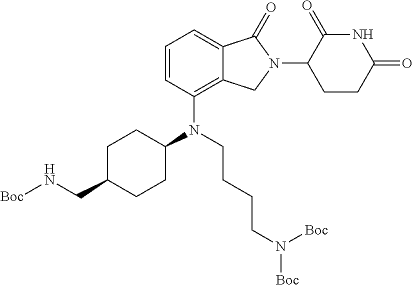 [SMILES: CC(C)(C)OC(=O)NC[C@H]1CC[C@@H](N(CCCCN(C(=O)OC(C)(C)C)C(=O)OC(C)(C)C)c2cccc3c2CN(C2CCC(=O)NC2=O)C3=O)CC1]

A mixture of tert-butyl (((1s,4s)-4-((2-(2,6-dioxopiperidin-3-yl)-1-oxoisoindolin-4-yl)amino)cyclohexyl)methyl)carbamate 3 (0.2 g, 0.425 mmol, 1.0 equiv) and tert-butyl (tert-butoxycarbonyl)(4-oxobutyl)carbamate 5 (0.244 g, 0.850 mmol, 2.0 equiv) in CH₂Cl₂ (10 ml) and DMF (10.00 ml) was added trifluoroacetic acid (0.131 ml, 1.700 mmol, 4.0 equiv) and stirred for 10 min at 0° C. Sodium triacetoxyborohydride (0.360 g, 1.700 mmol, 4.0 equiv) was added in portions at 0° C. and the resulted colourless cloudy mass was stirred for 16 h at room temperature (monitored by UPLC). Reaction mixture was cooled to 0° C. and additional quantity of tert-butyl (tert-butoxycarbonyl)(4-oxobutyl)carbamate 5 (0.244 g, 0.850 mmol, 2.0 equiv), trifluoroacetic acid (0.131 ml, 1.700 mmol, 4.0 equiv) were added and stirred for 10 min. Then, sodium triacetoxyborohydride (0.360 g, 1.700 mmol, 4.0 equiv) was added in portions at 0° C. and the resulted colourless cloudy mass was stirred for 16 hours at room temperature. Upon completion of the reaction (as confirmed by TLC analysis, 8: 2/EtOAc: pet ether, Rf˜0.6), the reaction mixture was quenched with ice cold water (20 ml) and extracted with dichloromethane (2×50 ml). The combined organic layer was dried over anhy. Na₂SO₄ and concentrated under vacuum to afford the crude product as a pale yellow liquid. The crude compound was purified by Isolera chromatography (Biotage R snap cartridge, KP-Sil, 100 g, 230-400 silica gel) using 60-70% of ethyl acetate in pet ether. The fractions were collected and concentrated under vacuum to get a pale yellow liquid.

The obtained compound was further purified by reverse phase chromatography (eluted with 75% of ACN in 0.10% HCO₂H), and the pure fraction was lyophilized to afford tert-butyl (tert-butoxycarbonyl)(4-(((1s,4s)-4-(((tert-butoxycarbonyl)amino)methyl)cyclohexyl) (2-(2,6-dioxopiperidin-3-yl)-1-oxoisoindolin-4-yl)amino)butyl)carbamate 6 (0.11 g, 0.146 mmol, 34% yield) as a white solid.

¹H-NMR (400 MHz, DMSO-d₆): δ 10.96 (s, 1H), 7.40-7.43 (m, 1H), 7.27-7.33 (m, 2H), 6.78-6.81 (m, 1H), 5.07-5.12 (m, 1H), 4.24-4.36 (m, 2H), 3.33-3.39 (m, 2H), 3.08-3.12 (m, 3H), 2.91-2.95 (m, 3H), 2.60-2.61 (m, 2H), 2.53-2.56 (m, 2H), 1.98-2.01 (m, 1H), 1.62-1.65 (m, 3H), 1.52-1.53 (m, 6H), 1.48 (m, 27H), 1.22-1.24 (m, 2H).

LCMS: 741.4 (M+), Rt (min):2.945, Area %—89.8%.

HPLC: Rt (min): 5.816; Area %—99.9%.

### Step-4: 3-(4-((4-aminobutyl)((1s,4s)-4-(aminomethyl)cyclohexyl)amino)-1-oxoisoindolin-2-yl)piperidine-2,6-dione dihydrochloride

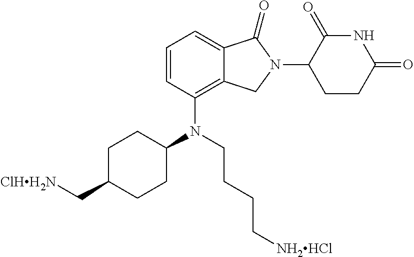

To a stirred solution of tert-butyl(tert-butoxycarbonyl)(4-(((1s,4s)-4-(((tert-butoxycarbonyl) amino)methyl)cyclohexyl)(2-(2,6-dioxopiperidin-3-yl)-1-oxoisoindolin-4-yl)amino)butyl) carbamate 6 (0.1 g, 0.135 mmol, 1.0 equiv) in dichloromethane (10 ml) was added HCl (4M soln. in dioxane, 10 ml) at 0° C. and then stirred at room temperature for 2 h. The progress of the reaction was monitored by LCMS. Reaction mixture was concentrated under vacuum at below 40° C., to afford off-white solid. The obtained solid was dissolved in water (10 ml) and washed with MTBE (2×10 ml), the water layer was lyophilized to afford 3-(4-((4-aminobutyl)((1s,4s)-4-(aminomethyl)cyclohexyl)amino)-1-oxoisoindolin-2-yl)piperidine-2,6-dione, 2HCl (62.5 mg, 0.116 mmol, 86% yield) as off-white solid.

¹H-NMR (400 MHz, DMSO-d6): δ 11.00 (s, 1H), 7.89-7.98 (m, 6H), 7.33-7.44 (m, 3H), 5.10-5.13 (m, 1H), 4.30-4.37 (m, 2H), 3.43-3.49 (m, 2H), 3.14-3.17 (m, 2H), 2.84-2.96 (m, 6H), 2.05 (m, 1H), 1.86 (m, 2H), 1.32-1.64 (m, 9H), 1.24 (m, 2H).

LCMS: 442.3 (M+H). Method: Column: XBridge C8 (50×4.6 mm) 3.5 μm, Mobile phase: A: 0.1% TFA in H₂O, Mobile phase: B: 0.1% TFA in Acetonitrile, Flow Rate: 1.5 ml/min.

HPLC: Method: Mobile Phase A: 0.1% TFA in water, Mobile Phase B: Methanol, Flow rate: 1.0 ml/min. Column: Atlantis dC18 (250×4.6) mm, 5 μm. RT: 6.488 min, Area:95.427%.

### Hydrochloride Salt Form of Compound 105

### 3-(4-((4-aminobutyl)((1R,4R)-4-(aminomethyl)cyclohexyl)amino)-1-oxoisoindolin-2-yl)piperidine-2,6-dione, 2HCl

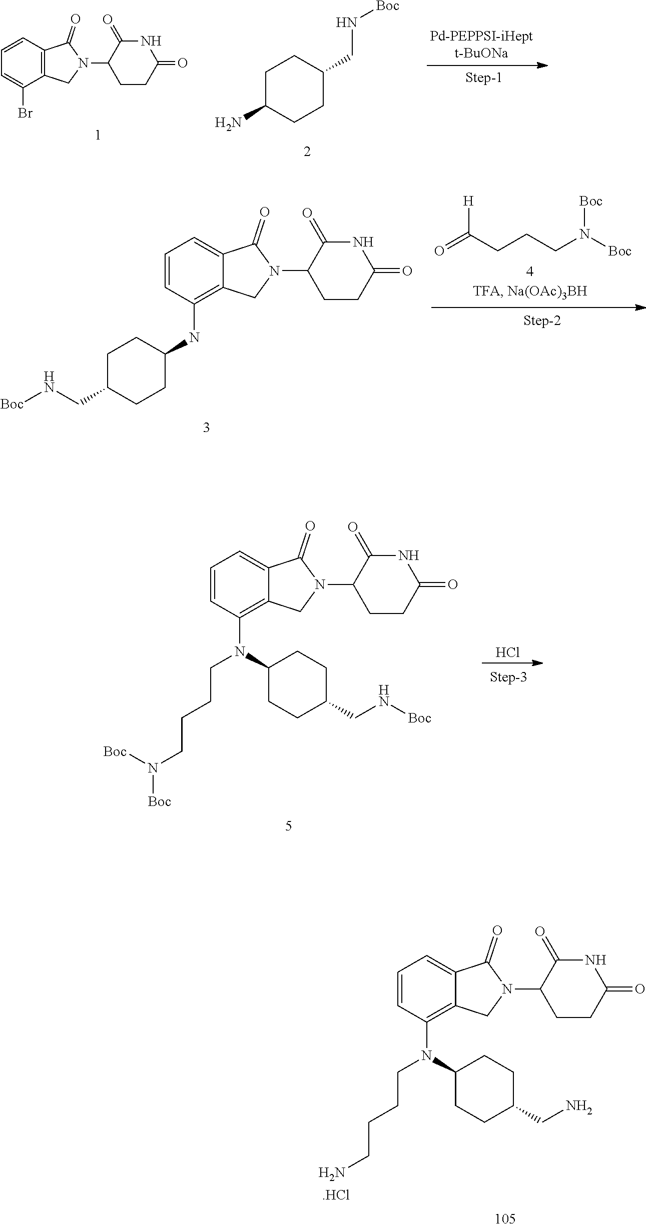

### Step-1: tert-butyl (((1R,4R)-4-((2-(2,6-dioxopiperidin-3-yl)-1-oxoisoindolin-4-yl)amino)cyclohexyl)methyl)carbamate

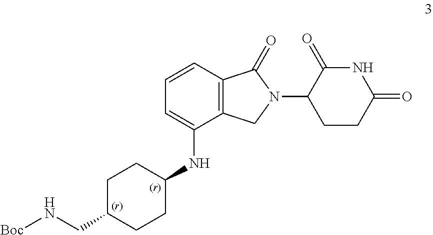 [SMILES: CC(C)(C)OC(=O)NC[C@H]1CC[C@H](Nc2cccc3c2CN(C2CCC(=O)NC2=O)C3=O)CC1]

3-(4-bromo-1-oxoisoindolin-2-yl)piperidine-2,6-dione 1 (1.0 g, 3.09 mmol, 1 equiv), tert-butyl (((1R,4R)-4-aminocyclohexyl)methyl)carbamate 2 (2.120 g, 9.28 mmol, 3 equiv) and NaOt-Bu (0.892 g, 9.28 mmol, 3 equiv) were dissolved in DMF (4 ml) and de-gassed for 20 min with nitrogen. Pd-PEPPSI-IHept(Cl) (0.151 g, 0.155 mmol, 0.05 equiv) was added under nitrogen atmosphere and heated the reaction at 110° C. for 4 h. Upon completion of the reaction (as confirmed by UPLC analysis, the reaction mixture was filtered through celite, washed with EtOAc (5 mL), concentrated to minimum volume (3 mL) and purified by reverse-phase column chromatography (Grace® column: C18 40 μm, 100 g; flow rate: 20 mL/min; 0.1% aqueous HCOOH/ACN mobile phase) to obtain tert-butyl (((1R,4R)-4-((2-(2,6-dioxopiperidin-3-yl)-1-oxoisoindolin-4-yl)amino)cyclohexyl)methyl)carbamate 3 (50 mg, 0.102 mmol, 3.30% yield) as a pale yellow solid.

LCMS: 401.1 (M-Boc). Method: Column: Atlantis dC18 (50×4.6 mm) 5 μm Mobile phase: A: 0.1% FA in H₂O B: ACN, Flow Rate: 1.5 mL/min. Rt (min): 2.262, Area %—96.25

¹H-NMR (400 MHz, DMSO-d6): δ 10.97 (s, 1H), 7.26 (t, J=7.60 Hz, 1H), 6.91 (d, J=7.60 Hz, 1H), 6.85-6.78 (m, 2H), 5.28 (d, J=8.40 Hz, 1H), 5.12 (dd, J=5.20, 13.20 Hz, 1H), 4.16 (d, J=17.2 Hz, 2H), 4.09 (d, J=17.2 Hz, 1H), 2.66 (t, J=14.40 Hz, 1H), 2.34-2.27 (m, 1H), 2.04-1.99 (m, 3H), 1.73-1.70 (m, 2H), 1.38 (s, 11H), 1.29-1.14 (m, 2H), 1.05-1.03 (m, 2H)

### Step-2: tert-butyl (tert-butoxycarbonyl)(4-(((1R,4R)-4-(((tert-butoxycarbonyl)amino)methyl)cyclohexyl)(2-(2,6-dioxopiperidin-3-yl)-1-oxoisoindolin-4-yl)amino)butyl)carbamate

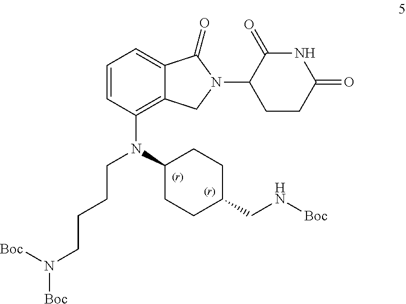 [SMILES: CC(C)(C)OC(=O)NC[C@H]1CC[C@H](N(CCCCN(C(=O)OC(C)(C)C)C(=O)OC(C)(C)C)c2cccc3c2CN(C2CCC(=O)NC2=O)C3=O)CC1]

To a solution of tert-butyl (((1R,4R)-4-((2-(2,6-dioxopiperidin-3-yl)-1-oxoisoindolin-4-yl) amino)cyclohexyl) methyl)carbamate 3 (90 mg, 0.191 mmol, 1 equiv), tert-butyl (tert-butoxycarbonyl)(4-oxobutyl)carbamate 4 (110 mg, 0.383 mmol, 2 equiv) DCM (5 ml) and DMF (2 mL) at 0° C. were added TFA (0.059 ml, 0.765 mmol, 4 equiv) and sodium triacetoxyborohydride (162 mg, 0.765 mmol, 4 equiv). The resulted reaction mixture was stirred at room temperature for 16 h. Upon completion of the reaction (as confirmed by LCMS), the reaction mixture was diluted with ice-cold water (10 mL), extracted with DCM (2×10 mL), washed with brine (25 mL), dried over Na₂SO₄, filtered and concentrated. The resulted residue was purified by reverse-phase column chromatography (Grace® column: C18 40 μm, 100 g; flow rate: 20 mL/min; 0.1% aqueous HCOOH/ACN mobile phase). The pure fractions from the column were lyophilized to afford tert-butyl (tert-butoxycarbonyl)(4-(((1R,4R)-4-(((tert-butoxycarbonyl)amino)methyl)cyclohexyl)(2-(2,6-dioxopiperidin-3-yl)-1-oxoisoindolin-4-yl)amino)butyl)carbamate 5 (20 mg, 0.025 mmol, 14.03% yield) as white solid.

LCMS: 742.5 (M+H). Method: Column: XBridge C8 (50×4.6 mm) 3.5 μm, Mobile phase: A: 0.1% TFA in H₂O, Mobile phase: B: 0.1% TFA in ACN, Flow Rate: 1.5 ml/min. Rt (min): 2.238; Area %—98.70.

¹H-NMR (400 MHz, DMSO-d₆): δ 10.97 (s, 1H), 7.40 (t, J=7.60 Hz, 1H), 7.26 (q, J=8.00 Hz, 2H), 6.83-6.82 (m, 1H), 5.09 (q, J=4.80 Hz, 1H), 3.12 (t, J=6.00 Hz, 2H), 3.00 (m, 2H), 2.90 (d, J=3.60 Hz, 2H), 1.70 (d, J=9.60 Hz, 4H), 1.69-1.38 (m, 4H), 1.24 (s, 33H), 1.19 (d, J=9.20 Hz, 2H), 0.87 (q, J=6.00 Hz, 2H).

### Step-3: 3-(4-((4-aminobutyl)((1R,4R)-4-(aminomethyl)cyclohexyl)amino)-1-oxoisoindolin-2-yl)piperidine-2,6-dione, 2HCl

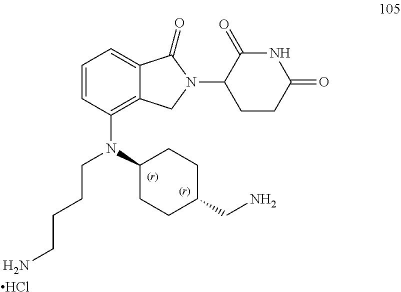 [SMILES: NCCCCN(c1cccc2c1CN(C1CCC(=O)NC1=O)C2=O)[C@H]1CC[C@H](CN)CC1 ; Cl]

To an ice cold solution of tert-butyl (tert-butoxycarbonyl)(4-(((1r,4r)-4-(((tert-butoxycarbonyl) amino)methyl)cyclohexyl)(2-(2,6-dioxopiperidin-3-yl)-1-oxoisoindolin-4-yl)amino)butyl) carbamate (20 mg, 0.027 mmol) 1 equiv) in DCM (1 mL) was added HCl (4M soln. in EtOAc, 1 mL, 4.0 mmol) and stirred for 2 h at room temperature. Upon completion of the reaction by TLC analysis (10% MeOH:DCM, R_{f}˜0.1), the reaction mixture was concentrated, and lyophilized to afford 3-(4-((4-aminobutyl)((1R,4R)-4-(aminomethyl)cyclohexyl)amino)-1-oxoisoindolin-2-yl)piperidine-2,6-dione, 2HCl (7.5 mg, 0.014 mmol, 52.3% yield) as a pale yellow solid.

LCMS: 428.3 (M+H) Method: Column: XBridge C8 (50×4.6 mm) 3.5 μm, Mobile phase: A: 0.1% TFA in H₂O, Mobile phase: B: 0.1% TFA in CAN, Flow Rate: 1.5 ml/min, Rt (min): 0.8/66; Area %—94.61.

HPLC: 96.75%, Rt (min): 2.674. Method: Method info: A: 0.1% TFA in H₂O, B: Methanol, Flow Rate:1.0 ml/min, COLUMN: YMC Hydrosphere C18 (150×4.6 mm), 3 μm

¹H-NMR (400 MHz, DMSO-d₆): δ 11.00 (s, 1H), 7.87-7.86 (m, 6H), 7.44 (s, 1H), 7.33 (s, 2H), 5.13-5.09 (m, 1H), 4.31 (q, J=16.40 Hz, 2H), 3.13 (s, 2H), 2.98 (t, J=6.00 Hz, 1H), 2.89 (d, J=5.20 Hz, 1H), 2.71 (s, 2H), 2.68 (d, J=1.60 Hz, 3H), 2.03 (d, J=10.80 Hz, 1H), 1.7-1.9 (m, 4H), 1.53 (t, J=7.20 Hz, 5H), 1.24-1.25 (m, 2H), 1.12-1.0 (m, 2H).

### Hydrochloride Salt Form of Compound 106

### 3-(4-((4-aminobutyl)(((1s,4s)-4-aminocyclohexyl)methyl)amino)-1-oxoisoindolin-2-yl)piperidine-2,6-dione, 2HCl

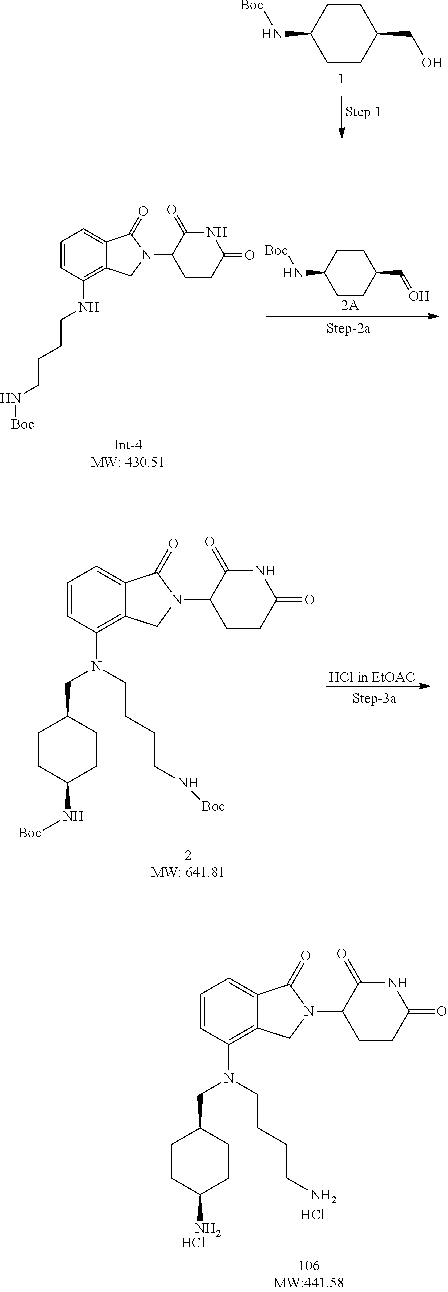

### Step-1: tert-butyl ((1s,4s)-4-formylcyclohexyl)carbamate

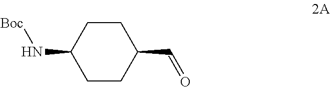 [SMILES: CC(C)(C)OC(=O)N[C@H]1CC[C@@H](C=O)CC1]

To a cooled solution of tert-butyl ((1s,4s)-4-(hydroxymethyl)cyclohexyl)carbamate (500 mg, 2.180 mmol, 1 equiv) in DCM (11 ml) was added DMSO (3.7 ml), DIPEA (1.510 ml, 8.48 mmol, 3.89 equiv) and a solution of pyridine sulfur trioxide (1.35 g, 8.48 mmol, 3.89 equiv) in DMSO (1.5 ml) in drops and was stirred at 0° C. for 1 h. Upon completion of reaction (as confirmed by TLC (40% ethyl acetate in pet ether, R_{f}: 0.4), the reaction was quenched with 1.5 N HCl (20 mL) and extracted with DCM (2×20 mL). The combined organic layer was dried over anhy. Na₂SO₄ and concentrated under vacuum to give the crude of tert-butyl ((1s,4s)-4-formylcyclohexyl)carbamate 2A (360 mg, 1.584 mmol, 72.6% yield) as a colorless liquid.

¹H-NMR (400 MHz, DMSO-d₆): δ 9.58 (s, 1H), 8.23 (s, 1H), 6.74 (s, 1H), 3.65-3.56 (m, 2H), 2.55-2.50 (m, 13H), 1.41 (s, 3H).

### Step-2: tert-butyl ((1s,4s)-4-(((4-((tert-butoxycarbonyl)amino)butyl)(2-(2,6-dioxopiperidin-3-yl)-1-oxoisoindolin-4-yl)amino)methyl)cyclohexyl)carbamate

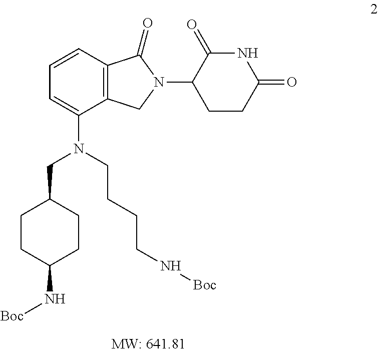 [SMILES: CC(C)(C)OC(=O)NCCCCN(C[C@H]1CC[C@@H](NC(=O)OC(C)(C)C)CC1)c1cccc2c1CN(C1CCC(=O)NC1=O)C2=O]

To a solution of tert-butyl (4-((2-(2,6-dioxopiperidin-3-yl)-1-oxoisoindolin-4-yl)amino)butyl)carbamate Int-4 (200 mg, 0.465 mmol, 1 equiv) and tert-butyl ((1r,4r)-4-formylcyclohexyl)carbamate 2A (528 mg, 2.323 mmol, 5 equiv) in DCE (2 ml) and DMF (0.5 ml) mixture was added TFA (47.7 mg, 0.418 mmol, 0.9 equiv) and Na(OAc)₃BH (35.0 mg, 0.557 mmol, 1.5 equiv) at 0° C. and was allowed to stir at room temperature for 12 h. Upon completion of the reaction (as confirmed UPLC) the reaction mixture was quenched with ice water (25 mL) and extracted with DCM (2×10 ml). The combined organic layer was concentrated under vacuum to get the crude product. The crude product was purified by reverse phase chromatography (Grace® column: generic C18, 40 g snap, 0.1% formic acid in ACN, 20 mL/min, 70-75% ACN in H₂O) and the product fraction was Lyophilised to give tert-butyl ((1r,4r)-4-(((4-((tert-butoxycarbonyl)amino)butyl)(2-(2,6-dioxopiperidin-3-yl)-1-oxoisoindolin-4-yl)amino)methyl)cyclohexyl)carbamate 2 (130 mg, 0.202 mmol, 43% yield) as off white solid.

¹H-NMR (400 MHz, DMSO-d6): δ 10.99 (s, 1H), 7.37 (t, J=7.60 Hz, 1H), 7.19 (d, J=7.20 Hz, 1H), 7.11 (d, J=8.00 Hz, 1H), 6.78 (t, J=5.20 Hz, 1H), 6.64 (d, J=6.80 Hz, 1H), 5.17-5.08 (m, 1H), 4.27 (d, J=17.20 Hz, 1H), 4.38 (d, J=16.80 Hz, 1H), 3.44 (s, 1H), 3.13 (dd, J=6.80, 28.00 Hz, 4H), 2.89 (t, J=6.00 Hz, 3H), 2.04 (q, J=4.80 Hz, 1H), 1.52 (t, J=21.20 Hz, 4H), 1.36 (d, J=9.20 Hz, 28H).

LCMS: 642.4 (M+H), Rt (min): 2.992, Area %: 99.757.

HPLC: Rt (min): 4.691, Area %: 99.938.

### Step-3: 3-(4-((4-aminobutyl)(((1s,4s)-4-aminocyclohexyl)methyl)amino)-1-oxoisoindolin-2-yl)piperidine-2,6-dione, 2HCl

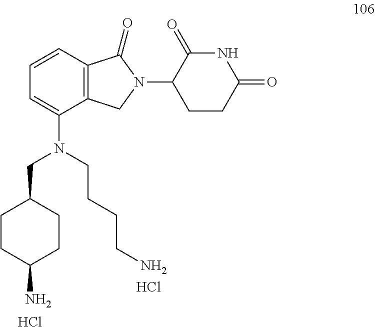 [SMILES: NCCCCN(C[C@H]1CC[C@@H](N)CC1)c1cccc2c1CN(C1CCC(=O)NC1=O)C2=O ; Cl ; Cl]

To an ice cold solution of tert-butyl ((1s,4s)-4-(((4-((tert-butoxycarbonyl)amino)butyl)(2-(2,6-dioxopiperidin-3-yl)-1-oxoisoindolin-4-yl)amino)methyl)cyclohexyl)carbamate (130 mg, 0.203 mmol, 1 equiv) in DCM (2.5 ml) was added HCl (4M in ethyl acetate, 1.3 mL, 5.20 mmol) at 0° C. and stirred for 2 h at room temperature. Upon completion of the reaction (as confirmed by LCMS), the reaction mixture was concentrated, washed with MTBE (10 mL) and dried to get 3-(4-((4-aminobutyl)(((1s,4s)-4-aminocyclohexyl)methyl)amino)-1-oxoisoindolin-2-yl)piperidine-2,6-dione, 2HCl 106 (85 mg, 0.165 mmol, 81% yield) as pale yellow solid.

¹H-NMR (400 MHz, DMSO-d6): δ 10.99 (s, 1H), 8.10 (d, J=35.60 Hz, 5H), 7.43 (s, 1H), 7.26 (s, 1H), 5.12 (q, J=5.20 Hz, 1H), 4.39 (q, J=16.80 Hz, 2H), 3.16 (d, J=26.00 Hz, 4H), 2.97-2.88 (m, 1H), 2.76 (d, J=4.80 Hz, 2H), 2.63 (s, 1H), 2.51 (t, J=1.60 Hz, 1H), 2.05 (t, J=5.20 Hz, 1H), 1.68 (s, 12H).

LCMS: 442.4 (M+H), Method: Mobile phase: A: 0.1% TFA in H₂O; Mobile phase: B: 0.1% TFA in ACN, Column: XBridge C8 (50×4.6 mm) 3.5 μm, Flow Rate: 1.5 ml/min, Rt (min): 1.027, Area %: 99.640.

HPLC: Method:A: 0.1% TFA in H₂O, B: ACN, Flow Rate: 2.0 ml/min, COLUMN: Xbridge C8(50×4.6) mm, 3.5 m Rt (min): 1.655, Area %: 99.741.

### Hydrochloride Salt Form of Compound 107

### 3-(4-((4-aminobutyl)(((1r,4r)-4-aminocyclohexyl)methyl)amino)-1-oxoisoindolin-2-yl)piperidine-2,6-dione, HCl

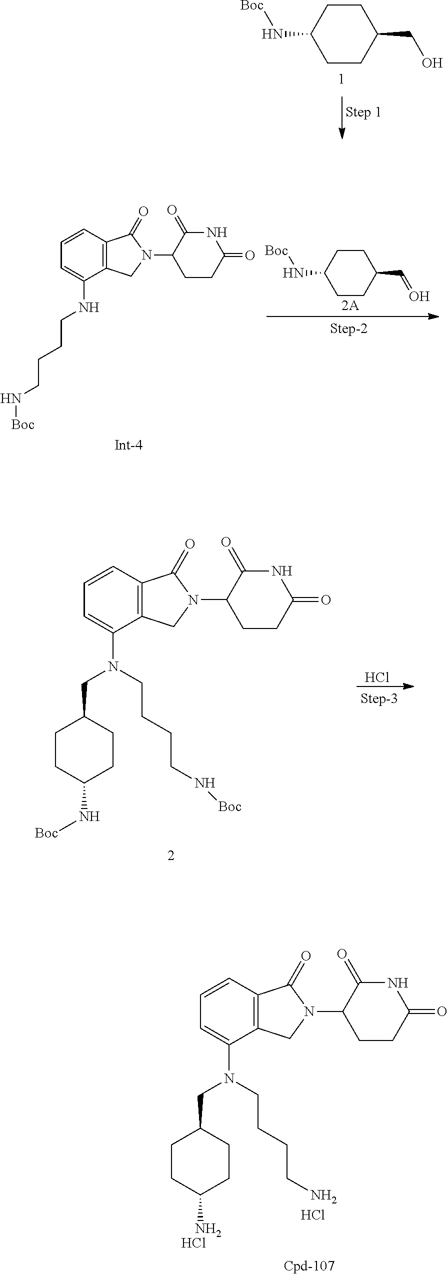

### Step-1: tert-butyl ((1r,4r)-4-formylcyclohexyl)carbamate 2A

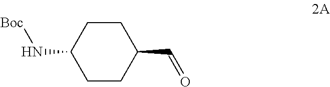 [SMILES: CC(C)(C)OC(=O)N[C@H]1CC[C@H](C=O)CC1]

To a cooled solution of tert-butyl ((1r,4r)-4-(hydroxymethyl)cyclohexyl)carbamate (500 mg, 2.180 mmol, 1 equiv) in DCM (11 ml) was added DMSO (3.7 ml), DIPEA (1.510 ml, 8.48 mmol, 3.89 equiv) followed by a solution of pyridine sulfur trioxide (1.35 g, 8.48 mmol, 3.89 equiv) in DMSO (3.7 ml) in drops. The mixture was stirred at 0° C. for 15 min. Upon completion of reaction (as confirmed by TLC (40% ethyl acetate in pet ether, R_{f}: 0.4), the reaction mixture was quenched with 1.5N HCl (20 mL) and extracted with DCM (2×20 mL). The combined organic layer was concentrated under vacuum to give the crude product of tert-butyl ((1r,4r)-4-formylcyclohexyl)carbamate 2A (500 mg, 2.123 mmol, 97% yield) as yellow solid

¹H-NMR (400 MHz, DMSO-d6): δ 9.55 (d, J=0.80 Hz, 1H), 6.75 (d, J=7.60 Hz, 1H), 3.16 (s, 1H), 2.52 (q, J=2.00 Hz, 3H), 2.09 (d, J=17.60 Hz, 2H), 1.92-1.83 (m, 5H), 1.38 (s, 1H), 1.19 (d, J=8.80 Hz, 5H).

LCMS: 172 (M-56), Rt (min): 2.587, Area %: 96.528.

### Step-2: tert-butyl ((1r,4r)-4-(((4-((tert-butoxycarbonyl)amino)butyl)(2-(2,6-dioxopiperidin-3-yl)-1-oxoisoindolin-4-yl)amino)methyl)cyclohexyl)carbamate

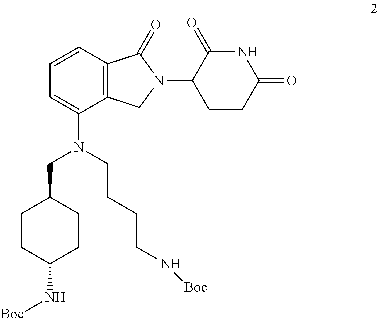 [SMILES: CC(C)(C)OC(=O)NCCCCN(C[C@H]1CC[C@H](NC(=O)OC(C)(C)C)CC1)c1cccc2c1CN(C1CCC(=O)NC1=O)C2=O]

To a solution of tert-butyl (4-((2-(2,6-dioxopiperidin-3-yl)-1-oxoisoindolin-4-yl)amino)butyl)carbamate Int4 (200 mg, 0.465 mmol, 1 equiv) and tert-butyl ((1r,4r)-4-formylcyclohexyl)carbamate 2A (528 mg, 2.323 mmol, 5 equiv) in DCE (2 ml) and DMF (0.5 ml) mixture was added TFA (47.7 mg, 0.418 mmol, 0.9 equiv) and Na(OAc)₃BH (35.0 mg, 0.557 mmol, 1.5 equiv) at 0° C. and was allowed to stir at room temperature for 12 h. Upon completion of the reaction (as confirmed UPLC) the reaction mixture was quenched with ice water (25 mL) and extracted with DCM (2×10 ml). The combined organic layer was concentrated under vacuum to give crude. The crude product was purified by Isolera (column size: Biotage R snap cartridge, KP-Sil, 25 g, Silica gel 230-400 mesh) using 50-60% ethyl acetate in pet ether to get tert-butyl ((1r,4r)-4-(((4-((tert-butoxycarbonyl)amino)butyl)(2-(2,6-dioxopiperidin-3-yl)-1-oxoisoindolin-4-yl)amino)methyl)cyclohexyl)carbamate 2 (60 mg, 0.093 mmol, 19.97% yield) as pale yellow solid.

¹H-NMR (400 MHz, DMSO-d6): δ 10.99 (s, 1H), 7.37 (t, J=7.60 Hz, 1H), 7.18 (d, J=7.20 Hz, 1H), 7.09 (d, J=8.00 Hz, 1H), 6.78 (t, J=5.60 Hz, 1H), 6.65 (d, J=8.00 Hz, 1H), 5.13-5.08 (m, 1H), 4.38 (d, J=16.80 Hz, 1H), 4.28 (d, J=16.80 Hz, 1H), 4.03 (q, J=7.20 Hz, 1H), 3.17 (d, J=6.80 Hz, 3H), 3.03 (d, J=6.80 Hz, 2H), 2.93-2.87 (m, 3H), 2.56 (t, J=2.00 Hz, 2H), 2.02 (t, J=9.20 Hz, 1H), 1.71 (d, J=8.80 Hz, 4H), 1.36 (d, J=2.00 Hz, 24H), 1.04-0.90 (m, 4H),

LCMS: 642.4 (M+H), Rt (min): 2.900 min, Area %: 99.216.

HPLC: Rt (min): 4.591, Area %: 97.770.

### Step-3:3-(4-((4-aminobutyl)(((1r,4r)-4-aminocyclohexyl)methyl)amino)-1-oxoisoindolin-2-yl)piperidine-2,6-dione, HCl

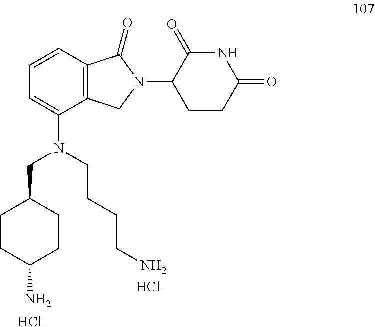 [SMILES: NCCCCN(C[C@H]1CC[C@H](N)CC1)c1cccc2c1CN(C1CCC(=O)NC1=O)C2=O ; Cl ; Cl]

To an ice cold solution tert-butyl ((1r,4r)-4-(((4-((tert-butoxycarbonyl)amino)butyl)(2-(2,6-dioxopiperidin-3-yl)-1-oxoisoindolin-4-yl)amino)methyl)cyclohexyl)carbamate 2 (60 mg, 0.093 mmol, of 1 equiv) in DCM (1.7 mL), was added HCl (4 M soln. in ethyl acetate, 0.89 ml, 3.56 mmol, 5 vol) and stirred for 6 h at room temperature. Upon completion of the reaction (as confirmed by UPLC), the reaction mixture was concentrated, washed with MTBE (10 mL) to give the crude which was lyophilised to afford 3-(4-((4-aminobutyl)(((1r,4r)-4-aminocyclohexyl)methyl)amino)-1-oxoisoindolin-2-yl)piperidine-2,6-dione, HCl (35 mg, 0.072 mmol, 77% yield) as pale yellow solid.

¹H-NMR (400 MHz, DMSO-d6): δ 11.00 (s, 1H), 7.98 (t, J=31.60 Hz, 6H), 7.40 (t, J=7.60 Hz, 1H), 7.20 (t, J=22.40 Hz, 2H), 5.12 (q, J=5.20 Hz, 1H), 4.35 (q, J=17.20 Hz, 2H), 3.20 (s, 2H), 3.06 (d, J=6.40 Hz, 2H), 2.96-2.90 (m, 2H), 2.88-2.68 (m, 2H), 2.61 (d, J=18.40 Hz, 2H), 2.04 (q, J=5.20 Hz, 1H), 1.21 (q, J=12.40 Hz, 2H), 0.98 (q, J=12.40 Hz, 2H).

LCMS: 441.0 (M-H), Method: Mobile phase: A: 0.1% FA in H₂O; Mobile phase: B: ACN; Column: Atlantis dC18 (50×4.6 mm) 5 μm; Flow Rate: 1.5 ml/min; Rt (min): 0.995, Area %: 98.488.

HPLC: Method: Mobile Phase A: 0.1% TFA in WATER; Mobile Phase B: Acetonitrile; Column: Atlantis dC18 (250×4.6) mm, 5 μm; Flow rate: 1.0 ml/min; Rt (min): 6.025, Area %: 97.654.

### Hydrochloride Salt Form of Compounds 108 and 109

### 3-(4-((4-Aminobutyl)amino)-1-oxoisoindolin-2-yl)piperidine-2,6-dione hydrochloride

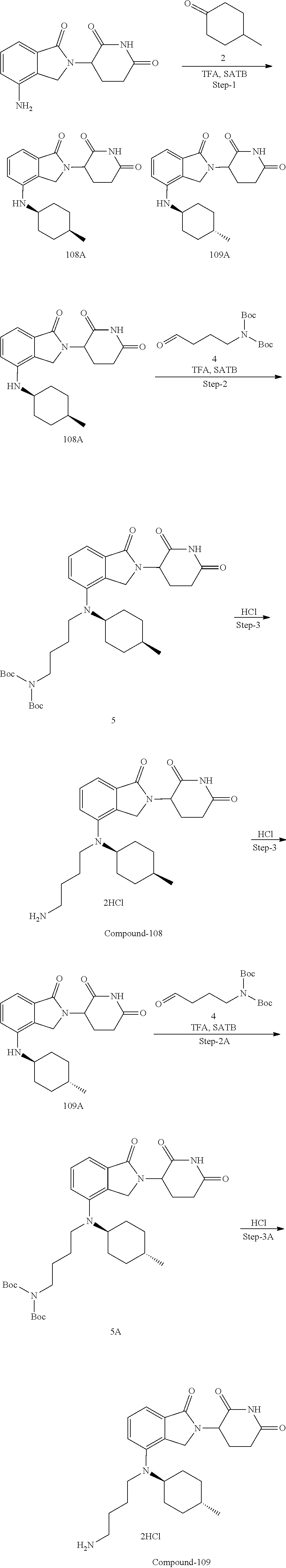

### Step-1: 3-(4-(((1s,4s)-4-methylcyclohexyl)amino)-1-oxoisoindolin-2-yl)piperidine-2,6-dione and 3-(4-(((1r,4r)-4-methylcyclohexyl)amino)-1-oxoisoindolin-2-yl)piperidine-2,6-dione

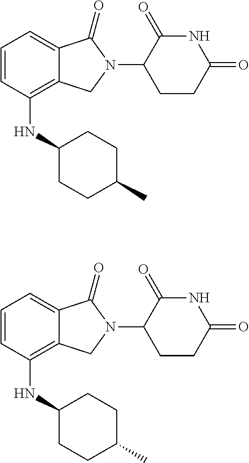 [SMILES: C[C@H]1CC[C@@H](Nc2cccc3c2CN(C2CCC(=O)NC2=O)C3=O)CC1 ; C[C@H]1CC[C@H](Nc2cccc3c2CN(C2CCC(=O)NC2=O)C3=O)CC1]

To a stirred solution of 3-(4-amino-1-oxoisoindolin-2-yl)piperidine-2,6-dione 1 (3 g, 11.57 mmol, 1.0 equiv) in TFA (30 ml) was added sodium triacetoxyhydroborate (12.26 g, 57.9 mmol, 5.0 equiv) and stirred at −15° C. for 10 min, then added 4-methylcyclohexan-1-one 2 (10.38 g, 93 mmol, 8.0 equiv) in DCM (10 ml), stirred the reaction for 16 h. The solvent was remove under reduced pressure to obtain crude. The crude compound was purified by reverse-phase column chromatography (Grace® column: C18 40 μm, 350 g; flow rate: 60 mL/min; 0.1% aqueous HCOOH/ACN mobile phase). Desired fraction was distilled under reduced pressure to obtain the cis and trans mixture 3-(4-((4-methylcyclohexyl)amino)-1-oxoisoindolin-2-yl)piperidine-2,6-dione (0.56 g, 13.62% yield) as off-white solid. The Cis-trans mixture was separated by prep-HPLC (Column: X-Select-C18-19×250 mm, Mobile phase: 0.1% TFA in Water/ACN, Flow-rate: 15 mL/min) to get 3-(4-(((1s,4s)-4-methylcyclohexyl)amino)-1-oxoisoindolin-2-yl)piperidine-2,6-dione 108A (0.29 g, 7.05% yield) as pale yellow solid and 3-(4-(((1r,4r)-4-methylcyclohexyl)amino)-1-oxoisoindolin-2-yl)piperidine-2,6-dione 109A (0.15 g, 3.65% yield) as pale-yellow solid.

### Step-2: tert-butyl (tert-butoxycarbonyl)(4-((2-(2,6-dioxopiperidin-3-yl)-1-oxoisoindolin-4-yl)((1s,4s)-4-methylcyclohexyl)amino)butyl)carbamate

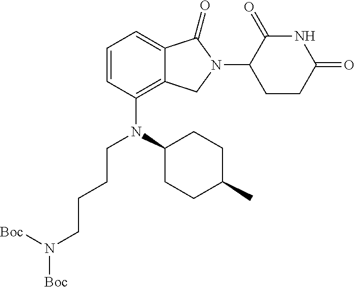 [SMILES: CC(C)(C)OC(=O)N(CCCCN(c1cccc2c1CN(C1CCC(=O)NC1=O)C2=O)[C@H]1CC[C@@H](C)CC1)C(=O)OC(C)(C)C]

To a stirred solution of 3-(4-(((1s,4s)-4-methylcyclohexyl)amino)-1-oxoisoindolin-2-yl)piperidine-2,6-dione 108A (0.098 g, 0.276 mmol, 1.0 equiv) and tert-butyl (tert-butoxycarbonyl)(4-oxobutyl)carbamate (0.475 g, 1.654 mmol, 6.0 equiv) in DCM (5 ml) and DMF (2 ml) was added TFA (0.189 g, 1.654 mmol, 6.0 equiv) and sodium triacetoxyhydroborate (0.497 g, 2.344 mmol, 8.5 equiv) at 0° C. The reaction mixture was warmed to RT and stirred for 16 h. Upon completion of the reaction (as confirmed by UPLC), the reaction mixture was quenched by ice-water (50 mL) and extracted with DCM (2×30 mL). The combined organic layer was dried with sodium sulphate, filtered and concentrated under reduced pressure to afford crude product. The crude product was purified by reverse-phase column chromatography (Grace® column: C18 40 μm, 120 g; flow rate: 20 mL/min; 0.1% aqueous HCOOH/ACN mobile phase) to afford desired product tert-butyl (tert-butoxycarbonyl)(4-((2-(2,6-dioxopiperidin-3-yl)-1-oxoisoindolin-4-yl)((1s,4s)-4-methylcyclohexyl)amino)butyl)carbamate (0.07 g, 39.7% yield) as off-white solid.

¹H-NMR (400 MHz, DMSO-d6): δ 10.98 (s, 1H), 7.42 (t, J=7.60 Hz, 1H), 7.34-7.27 (m, 2H), 5.13-5.08 (m, 1H), 4.36-4.25 (m, 2H), 3.41-3.38 (m, 2H), 3.13-3.10 (m, 3H), 2.94-2.86 (m, 1H), 2.68-2.67 (m, 2H), 2.01-1.98 (m, 1H), 1.72-1.64 (m, 3H), 1.48-1.44 (m, 25H), 1.38-1.23 (m, 3H), 0.94-0.92 (m, 3H),

LCMS: 627.4 (M+H), Rt (min): 3.41, Area %—97.91. HPLC: Rt (min): 4.88, Area: 99.57%.

### Step-3: 3-(4-((4-aminobutyl)((1s,4s)-4-methylcyclohexyl)amino)-1-oxoisoindolin-2-yl)piperidine-2,6-dione hydrochloride

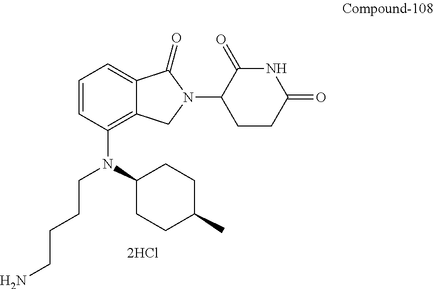 [SMILES: C[C@H]1CC[C@@H](N(CCCCN)c2cccc3c2CN(C2CCC(=O)NC2=O)C3=O)CC1]

To an ice cold suspension of tert-butyl (tert-butoxycarbonyl)(4-((2-(2,6-dioxopiperidin-3-yl)-1-oxoisoindolin-4-yl)((1s,4s)-4-methylcyclohexyl)amino)butyl)carbamate 5 (0.07 g, 0.112 mmol, 1.0 equiv) in DCM (3 ml) was added HCl (4M soln. EtOAc, 1 mL) at 0° C. The reaction mixture was stirred at RT for 2 h. The reaction mixture was concentrated and washed with methyl tert-butyl ether (5 mL). The solvent was decanted and concentrated under reduced pressure to afford the crude product. The crude product was lyophilized to obtain 3-(4-((4-aminobutyl)((1s,4s)-4-methylcyclohexyl)amino)-1-oxoisoindolin-2-yl)piperidine-2,6-dione, 2HCl 108 (0.06 g, 106% yield) as off-white solid.

¹H-NMR (400 MHz, DMSO-d₆): δ 10.99 (s, 1H), 7.81 (br s, 4H), 7.34-7.44 (m, 2H), 5.12 (t, J=8.40 Hz, 1H), 4.31-4.35 (m, 2H), 3.13 (br s, 2H), 2.92 (s, 1H), 2.60-2.71 (m, 3H), 2.01-2.05 (m, 1H), 1.69-1.77 (m, 4H), 1.57-1.63 (m, 8H), 1.24-1.31 (m, 2H), 0.93-0.94 (m, 3H).

LCMS: 427.3 (M+H). Method: Mobile Phase A: 0.1% TFA in H₂O. Mobile Phase B: Acetonitrile. Flow rate:1.5 ml/min. COLUMN: XBridge C8 (50×4.6 mm) 3.5 μm. Rt (min): 1.340; Area %—99.13.

HPLC Method: Mobile Phase A: 0.1% TFA in H₂O. Mobile Phase B: Acetonitrile. Flow rate: 2.0 mL/min. COLUMN: Xbridge C8(50×4.6) mm, 3.5 μm. Rt (min): 2.38; Area %—98.95.

### Step-2A: tert-butyl (tert-butoxycarbonyl)(4-((2-(2,6-dioxopiperidin-3-yl)-1-oxoisoindolin-4-yl)((1r,4r)-4-methylcyclohexyl)amino)butyl)carbamate

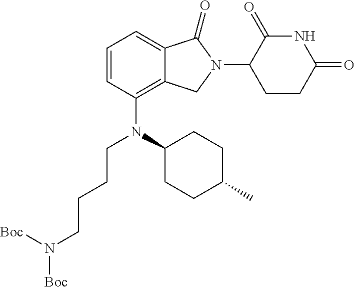 [SMILES: CC(C)(C)OC(=O)N(CCCCN(c1cccc2c1CN(C1CCC(=O)NC1=O)C2=O)[C@H]1CC[C@H](C)CC1)C(=O)OC(C)(C)C]

To a stirred solution of 3-(4-(((1r,4r)-4-methylcyclohexyl)amino)-1-oxoisoindolin-2-yl)piperidine-2,6-dione, 109A (0.1 g, 0.281 mmol, 1.0 equiv) and tert-butyl (tert-butoxycarbonyl)(4-oxobutyl)carbamate (0.162 g, 0.563 mmol, 2.0 equiv) in DMF (5 ml): DCM (5 ml) was added TFA (0.160 g, 1.407 mmol, 4.0 equiv). After 10 minutes, sodium triacetoxyhydroborate (0.239 g, 1.125 mmol, 4.0 equiv) was added portionwise to this mixture at 0° C. The reaction mixture was stirred for 4 hour at room temperature. The completion of reaction was monitored by UPLC, which indicate 39% of SM. The reaction mixture was cooled down at 0° C. and added tert-butyl (tert-butoxycarbonyl)(4-oxobutyl)carbamate (0.485 g, 1.688 mmol, 6.0 equiv), followed by addition of TFA (0.160 g, 1.407 mmol, 5.0 equiv). After 10 minutes, sodium triacetoxyhydroborate (0.507 g, 2.391 mmol, 8.5 equiv) was added and stirred the reaction mixture for 16 hour at room temperature. The completion of reaction was monitored by UPLC. The reaction mixture was quenched by ice-water (20 mL) and extracted with DCM (3×20 mL). The combined organic layer was dried with sodium sulphate, filtered and concentrated under reduced pressure to afford crude product. The crude compound was purified by flash column chromatography (silica-gel, 230-400 mesh size) using ethyl acetate/pet ether (50%) as an eluent to obtain desired product as off-white solid. The product was repurified by reverse-phase column chromatography (Grace column: C18 40 μm, 50 g; flow rate: 20 mL/min; 0.1% aqueous HCOOH/ACN mobile phase) to afford tert-butyl (tert-butoxycarbonyl)(4-((2-(2,6-dioxopiperidin-3-yl)-1-oxoisoindolin-4-yl)((1r,4r)-4-methylcyclohexyl)amino)butyl)carbamate 5A (0.04 g, 20.87% yield) as off-white solid.

¹H-NMR (400 MHz, DMSO-d₆): δ 10.98 (s, 1H), 7.42-7.38 (m, 1H), 7.30-7.23 (m, 2H), 5.11-5.07 (m, 1H), 4.34-4.23 (m, 2H), 3.12-3.11 (m, 2H), 2.86-3.01 (m, 2H), 2.65-2.70 (m, 1H), 2.01-1.98 (m, 1H), 1.69-1.66 (m, 4H), 1.53-1.46 (m, 5H), 1.37 (s, 18H), 1.22-1.33 (m, 3H), 0.99-0.91 (m, 2H), 0.85-0.83 (m, 3H).

LCMS: 627.3 (M-H), Rt (min): 2.27, Area %—92.32.

### Step-3A: 3-(4-((4-aminobutyl)((1r,4r)-4-methylcyclohexyl)amino)-1-oxoisoindolin-2-yl)piperidine-2,6-dione hydrochloride

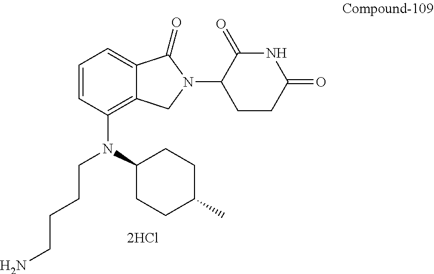 [SMILES: C[C@H]1CC[C@H](N(CCCCN)c2cccc3c2CN(C2CCC(=O)NC2=O)C3=O)CC1]

To an ice cold solution of tert-butyl (tert-butoxycarbonyl)(4-((2-(2,6-dioxopiperidin-3-yl)-1-oxoisoindolin-4-yl)((1r,4r)-4-methylcyclohexyl)amino)butyl)carbamate 5A (0.04 g, 0.064 mmol, 1.0 equiv) DCM (3 ml) was added HCl (4M soln. in EtOAc, 1 mL) at 0° C. stirred the reaction mixture for 2 hour at room temperature. Upon completion of the reaction (as confirmed by TLC analysis, 100% EtOAc R_{f}˜0.3, and LCMS). The reaction mixture was taken for vacuum distillation to get the crude product. The crude product was washed with MTBE (5 mL), decant the solvent dried it under reduced pressure and lyophilized to afford desired product 109 3-(4-((4-aminobutyl)((1r,4r)-4-methylcyclohexyl)amino)-1-oxoisoindolin-2-yl)piperidine-2,6-dione hydrochloride (0.03 g, 101% yield) as off-white solid.

¹H-NMR (400 MHz, DMSO-d₆): δ 11.01 (s, 1H), 7.99 (brs, 3H), 7.37-7.51 (m, 3H), 5.10-5.15 (m, 1H), 4.44-4.63 (m, 2H), 3.30 (s, 2H), 3.01 (s, 3H), 2.92-2.98 (m, 2H), 2.60-2.78 (m, 3H), 2.45-2.50 (m, 1H), 2.06-2.08 (m, 1H), 1.66-1.67 (m, 4H).

LCMS: 345.1 (M+H). Method: Mobile Phase A: 0.1% TFA in H₂O. Mobile Phase B: Acetonitrile. Flow rate:1.5 ml/min. COLUMN: XBridge C8 (50×4.6 mm) 3.5 μm. Rt (min): 1.351; Area %—99.19.

HPLC Method: Mobile Phase A: 0.1% TFA in H₂O. Mobile Phase B: Acetonitrile. Flow rate: 2.0 mL/min. COLUMN: Xbridge C8(50×4.6) mm, 3.5 μm. Rt (min): 2.418; Area %—99.64.

### Hydrochloride Salt Form of Compound 115

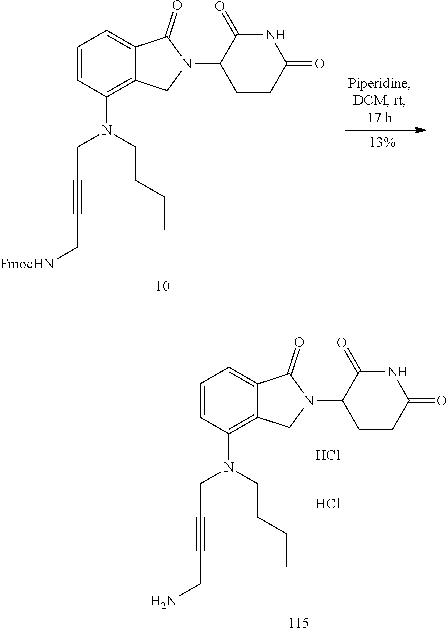

To a solution of compound 10 (120 mg, 0.2 mmol, 1.0 eq.) in DCM (6.0 mL) were added piperidine (338 mg, 4.0 mmol, 20.0 eq.). The mixture was stirred at room temperature for 17 hours. Then the reaction mixture was concentrated under reduced pressure and purified by P-HPLC (acetonitrile/H₂O: 20%-45%) to afford compound 115 (11.4 mg, 13%) as white solid.

TLC: DCM/methanol=10/1

R_{f} (Compound 10)=0.9

R_{f} (Compound 115)=0.1

LCMS: Calcd. for [C₂₁H₂₆N₄O₃]+: 383, Found: 383.

¹H NMR (400 MHz, D₂O): δ 7.72 (d, J=7.3 Hz, 1H), 7.66 (d, J=7.9 Hz, 1H), 7.60 (t, J=7.7 Hz, 1H), 5.06 (dd, J=13.3, 4.9 Hz, 1H), 4.53 (q, J=17.5 Hz, 2H), 4.27 (s, 2H), 3.60 (s, 2H), 3.50-3.35 (m, 2H), 2.97-2.62 (m, 2H), 2.47-2.30 (m, 1H), 2.25-2.10 (m, 1H), 1.37-1.20 (m, 2H), 1.13 (dd, J=14.5, 7.2 Hz, 2H), 0.65 (t, J=7.2 Hz, 3H).

### Hydrochloride Salt Form of Compound 116

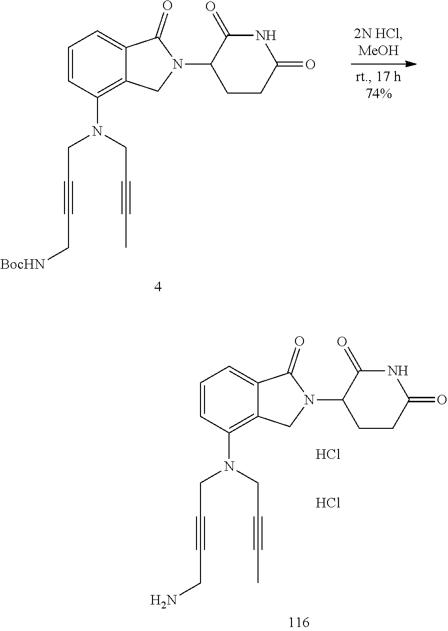 [SMILES: CC#CCN(CC#CCNC(=O)OC(C)(C)C)c1cccc2c1CN(C1CCC(=O)NC1=O)C2=O ; CC#CCN(CC#CCN)c1cccc2c1CN(C1CCC(=O)NC1=O)C2=O ; Cl ; Cl]

To a solution of compound 4 (20 mg, 0.04 mmol, 1.0 eq.) in MeOH (1 mL) were added HCl (0.2 mL, 2 N, 0.4 mmol, 10.0 eq.). The mixture was stirred at room temperature for 24 hours and concentrated under reduced pressure. The residue was purified by Biotage (C18, acetonitrile/H₂O: 30%-60%) to afford compound 116 (14.0 mg, 74%) as white solid.

TLC: DCM/methanol=10/1

R_{f} (Compound 4)=0.5

R_{f} (Compound 116)=0.1

LCMS: Calcd. for [C₂₁H₂₆N₄O₃]⁺: 383, Found: 383.

¹H NMR (400 MHz, D₂O): δ 8.30-8.20 (m, 2H), 7.42-7.25 (m, 3H), 4.98 (d, J=12.6 Hz, 1H), 4.38 (q, J=17.2 Hz, 2H), 3.67 (s, 2H), 3.11-2.98 (m, 2H), 2.81-2.62 (m, 4H), 2.42-2.22 (m, 1H), 2.15-2.01 (m, 1H), 1.53 (s, 3H), 1.50-1.28 (m, 4H).

### Hydrochloride Salt Form of Compound 123

### 3-(4-((4-amino-3,3-dimethylbutyl)(pentyl)amino)-1-oxoisoindolin-2-yl)piperidine-2,6-dione hydrochloride

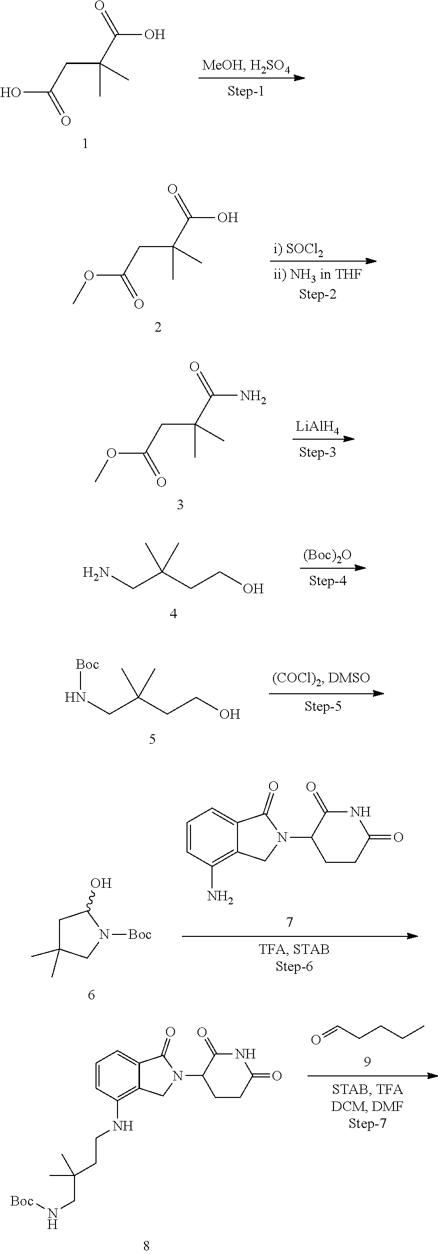

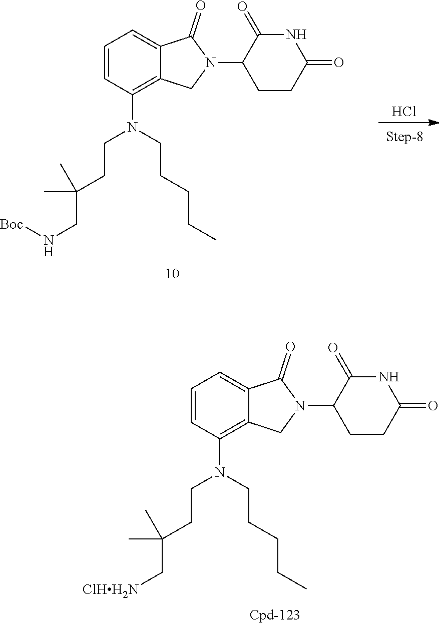

### Step-1: 4-Methoxy-2,2-dimethyl-4-oxobutanoic acid

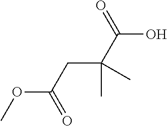

To an ice cold solution of 2,2-dimethylsuccinic acid 1 (100 g, 684 mmol, 1 equiv) in methanol (1500 mL, 15 vol), was added concentrated. H₂SO₄ (10 mL) dropwise and stirred for 16 hours at room temperature. Upon completion of the reaction (as confirmed by TLC analysis, 40% EtOAc in pet ether, R_{f}˜0.5, KMnO₄), the reaction mixture was concentrated under vacuum at 30° C. The resulted residue was poured into ice cold solution of sat. NaHCO₃ solution (1000 mL) and washed with EtOAc (2×500 mL). The separated aqueous layer was further acidified with aq. HCl (6N) till pH˜2 and extracted with EtOAc (2×800 mL). The combined organic extracts were washed with brine (1×600 mL), dried over Na₂SO₄, filtered and concentrated to afford 4-Methoxy-2,2-dimethyl-4-oxobutanoic acid 2 (40 g, 235 mmol, 34.3% yield) as colorless liquid.

¹H-NMR (400 MHz, DMSO-d₆): δ 12.17 (s, 1H), 3.57 (s, 3H), 2.53 (s, 2H), 1.18 (s, 6H).

### Step-2: Methyl 4-amino-3,3-dimethyl-4-oxobutanoate

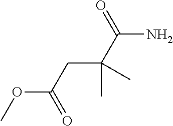

To an ice cold solution of 4-Methoxy-2,2-dimethyl-4-oxobutanoic acid 2 (25 g, 156 mmol, 1 equiv) in DCM (250 mL, 10 vol) was added thionyl chloride (111 g, 937 mmol, 6 equiv) dropwise and stirred for 3 h at room temperature. Upon completion of the reaction (as confirmed by TLC analysis, 20% EtOAc in pet ether, R_{f}˜0.4, as methyl ester), the reaction mixture was distilled under vacuum and dried completely under reduced pressure.

The resulted acid chloride mixture was dissolved in THF (250 mL) and added dropwise to a pre-cooled solution of NH₃ (0.5M soln. in THF, 375 mL) at −78° C. The reaction mixture was warmed to room temperature and stirred for 30 minutes. Upon completion of the reaction (as confirmed by TLC analysis, 60% EtOAc in pet ether, R_{f}˜0.2), the reaction mixture was concentrated and resulted residue was poured into ice cold solution of sat. NaHCO₃ solution (500 mL) and extracted with EtOAc (2×500 mL). The combined organic layer was washed with brine (500 mL), dried over Na₂SO₄, filtered and concentrated to afford methyl 4-amino-3,3-dimethyl-4-oxobutanoate 3 (8 g, 50.3 mmol, 32.2% yield) as pale yellow liquid.

¹H-NMR (400 MHz, DMSO-d₆): δ 6.75-7.07 (m, 2H), 3.54 (s, 3H), 2.51 (s, 2H), 1.15 (s, 6H)

### Step-3: 4-Amino-3,3-dimethylbutan-1-ol

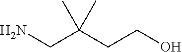

A solution of Methyl 4-amino-3,3-dimethyl-4-oxobutanoate 3 (13 g, 82 mmol, 1 equiv) in THF (100 mL, 8 vol) was treated with LiAlH₄ (2M in THF, 82 mL, 163 mmol, 2 equiv) dropwise at 0° C. over a period of 30 minutes. Once the addition was complete, the reaction mixture was warmed to room temperature, slowly heated to 65° C. and stirred for 2 h. Upon completion of the reaction (as confirmed by TLC analysis, 100% EtOAc, R_{f}˜0.1), the reaction mixture was cooled to 0° C. and slowly quenched with saturated Na₂SO₄ solution (30 ml), precipitated inorganic salts were removed by filtration through Celite pad and washed with EtOAc (1×100 mL). The combined filtrate was dried over Na₂SO₄, filtered and concentrated to afford 4-amino-3,3-dimethylbutan-1-ol 4 (7.8 g, 64.0 mmol, 78% yield) as a pale brown liquid, which was used without further purification.

¹H-NMR (400 MHz, DMSO-d₆): δ 3.40-3.43 (m, 2H), 2.30 (s, 2H), 1.35-1.38 (m, 2H), 0.80 (s, 6H).

### Step-4: tert-Butyl (4-hydroxy-2,2-dimethylbutyl)carbamate

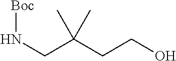

To a solution of 4-Amino-3,3-dimethylbutan-1-ol 4 (7.5 g, 57.2 mmol, 1 equiv) in THF (150 mL, 20 vol) and water (40 mL, 5 vol) were added sodium bicarbonate (7.2 g, 86 mmol, 1.5 equiv) and Boc₂O (14 mL, 57.2 mmol, 1 equiv) in drops at room temperature. The resulted reaction mixture was stirred for 16 hours at room temperature. Upon completion of the reaction (as confirmed by TLC analysis, 100% EtOAc, R_{f}˜0.7), the reaction mixture was diluted with EtOAc (1×100 mL), layer separated, extracted with ethyl acetate (2×100 mL). The combined organic layer was washed with brine solution (1×100 mL) and dried over Na₂SO₄ and concentrated under reduced pressure to afford as a pale brown liquid. The resulted residue was purified by chromatography (column size: Biotage R snap cartridge, KP-Sil, 50 g, 100-200 silica gel) using 40-50% EtOAc in pet ether to afford tert-butyl (4-hydroxy-2,2-dimethylbutyl) carbamate 5 (5.0 g, 22.85 mmol, 40.0% yield) as off-white crystalline solid.

¹H-NMR (400 MHz, DMSO-d₆): δ 6.70 (s, 1H), 4.28 (t, J=6.40 Hz, 1H), 3.41-3.47 (m, 2H), 2.76 (d, J=8.80 Hz, 2H), 1.43 (s, 9H), 0.83 (s, 6H).

### Step-5: tert-Butyl 2-hydroxy-4,4-dimethylpyrrolidine-1-carboxylate

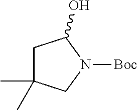

A solution of oxalyl chloride (0.5 mL, 5.52 mmol, 1.2 equiv) in DCM (5 mL) was added DMSO (1 mL, 14.08 mmol) dropwise at −78° C. and stirred for 15 minutes at same temperature. To this, tert-Butyl (4-hydroxy-2,2-dimethylbutyl)carbamate (1.0 g, 4.6 mmol, 1 equiv) in DCM (3 mL) was added dropwise over a period of 15 minutes and stirred for 30 minutes at −78° C. At this point, N,N-Diisopropylethylamine (2.4 mL, 13.81 mmol, 3 equiv) was added dropwise to the mixture at −78° C. and slowly warmed to 0° C. After stirred for 30 minutes at 0° C., the reaction completion was confirmed by TLC analysis (20% EtOAc in pet ether, R_{f}˜0.6, KMnO₀₄). The reaction mixture was diluted with DCM (20 mL) and washed with 10% aq citric acid solution (1×50 mL), water (1×30 mL) and brine (1×30 mL), dried over Na₂SO₄, filtered and concentrated under vacuum below 30° C. to afford tert-Butyl 2-hydroxy-4,4-dimethylpyrrolidine-1-carboxylate (0.95 g, crude) as pale yellow liquid which was used further without purification. GCMS: Mass found; MS: 215.1; Rt (min): 3.005. Method: HP5_SCAN350_MS.amx

### Step-6: tert-butyl (4-((2-(2,6-dioxopiperidin-3-yl)-1-oxoisoindolin-4-yl)amino)-2,2-dimethylbutyl)carbamate

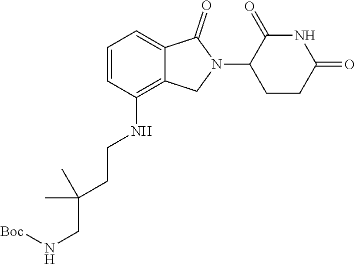 [SMILES: CC(C)(CCNc1cccc2c1CN(C1CCC(=O)NC1=O)C2=O)CNC(=O)OC(C)(C)C]

To a stirred mixture of 3-(4-amino-1-oxoisoindolin-2-yl)piperidine-2,6-dione (1.0 g, 3.86 mmol, 1.0 equiv) and tert-butyl 2-hydroxy-4,4-dimethylpyrrolidine-1-carboxylate (0.996 g, 4.63 mmol, 1.2 equiv) in CH₂Cl₂ (10 ml) and DMF (10.00 ml) was added trifluoroacetic acid (1.189 ml, 15.43 mmol, 4.0 equiv) and stirred for 10 min at 0° C. To this mixture, sodium triacetoxyborohydride (3.27 g, 15.43 mmol, 4.0 equiv) was added in portions at 0° C. and the resulted colourless cloudy mass was stirred for 16 hours at room temperature. Upon completion of the reaction (as confirmed by TLC and LCMS analysis, 100% EtOAc, Rf˜0.7), the reaction mixture was quenched with ice cold water (20 ml) and extracted with dichloromethane (2×50 ml). The combined organic layer was dried over Na₂SO₄ and concentrated under vacuum to afford crude as a pale yellow liquid. The crude compound was purified by Isolera chromatography (column size: Biotage R snap cartridge, KP-Sil, 100 g, 100-200 silica gel) using 75-85% of ethyl acetate in pet ether. The fractions were collected and contracted under vacuum to afford tert-butyl (4-((2-(2,6-dioxopiperidin-3-yl)-1-oxoisoindolin-4-yl)amino)-2,2-dimethylbutyl)carbamate 8 (0.6 g, 1.049 mmol, 27.2% yield) as pale yellow semi solid.

¹H-NMR (400 MHz, DMSO-d₆): δ 11.02 (s, 1H), 7.29 (t, J=10.40 Hz, 1H), 6.93 (d, J=10.00 Hz, 1H), 6.82-6.86 (m, 1H), 6.75 (d, J=10.80 Hz, 1H), 5.49 (s, 1H), 5.08-5.15 (m, 1H), 4.08-4.25 (m, 2H), 3.13-3.14 (m, 2H), 2.73-2.91 (m, 3H), 2.65 (m, 1H), 2.30-2.36 (m, 1H), 2.01-2.08 (m, 1H), 1.45-1.50 (m, 2H), 1.38 (s, 9H), 0.94 (s, 6H). LC-MS: 403.1 (M-tBu), Rt (min): 2.302, Area %—80.229.

### Step-7: tert-butyl (4-((2-(2,6-dioxopiperidin-3-yl)-1-oxoisoindolin-4-yl)(pentyl)amino)-2,2-dimethylbutyl)carbamate

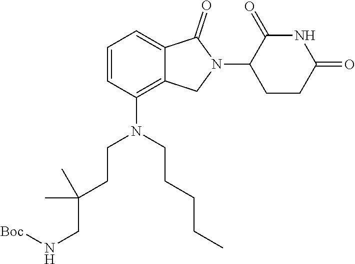 [SMILES: CCCCCN(CCC(C)(C)CNC(=O)OC(C)(C)C)c1cccc2c1CN(C1CCC(=O)NC1=O)C2=O]

To a stirred mixture of tert-butyl (4-((2-(2,6-dioxopiperidin-3-yl)-1-oxoisoindolin-4-yl)amino)-2,2-dimethylbutyl)carbamate 8 (0.3 g, 0.654 mmol, 1.0 equiv) and pentanal 9 (0.564 g, 6.54 mmol, 10.0 equiv) in CH₂Cl₂ (5 ml) and DMF (5.00 ml) was added trifluoroacetic acid (0.202 ml, 2.62 mmol, 4.0 equiv) and stirred for 10 min at 0° C. Sodium triacetoxyborohydride (0.555 g, 2.62 mmol, 4.0 equiv) was added in portions at 0° C. and the resulted colourless cloudy mass was stirred for 16 h at room temperature. Upon completion of the reaction (as confirmed by TLC and LCMS analysis, 100% EtOAc, Rf˜0.8), the reaction mixture was quenched with ice cold water (20 ml) and extracted with dichloromethane (2×50 ml). The combined organic layer was dried over Na₂SO₄ and concentrated under vacuum to afford crude as a pale yellow liquid. The crude compound was purified by prep-HPLC (Column: X-Bridge-c18 19.1×250, Mobile phase: 0.1% FA in Water/ACN, FLOW rate:15 ml/min) to afford tert-butyl (4-((2-(2,6-dioxopiperidin-3-yl)-1-oxoisoindolin-4-yl)(pentyl)amino)-2,2-dimethylbutyl)carbamate 10 (80 mg, 0.145 mmol, 22.21% yield) as a off-white solid.

¹H-NMR (400 MHz, DMSO-d6): δ 10.99 (s, 1H), 7.34-7.39 (m, 1H), 7.16-7.18 (m, 1H), 6.82-7.05 (m, 2H), 5.09-5.13 (m, 1H), 4.25-4.42 (m, 2H), 3.16-3.34 (m, 4H), 2.87-2.92 (m, 1H), 2.75-2.77 (m, 3H), 2.02 (m, 1H), 1.36 (m, 9H), 1.25 (s, 9H), 0.80-0.83 (m, 9H).

LCMS: 529.3 (M+H), Rt (min): 2.715, Area % —99.791.

### Step-8: 3-(4-((4-amino-3,3-dimethylbutyl)(pentyl)amino)-1-oxoisoindolin-2-yl)piperidine-2,6-dione hydrochloride

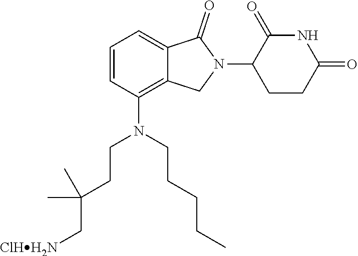

To a stirred suspension of tert-butyl (4-((2-(2,6-dioxopiperidin-3-yl)-1-oxoisoindolin-4-yl)(pentyl)amino)-2,2-dimethylbutyl)carbamate 10 (80 mg, 0.151 mmol) in dichloromethane (2 mL) was added HCl (4M in ethylacetate, 5 ml, 165 mmol) at 0° C. and then stirred at room temperature for 16 h. Upon completion of the reaction (as confirmed by TLC analysis, 100% EtOAc, Rf˜0.1), the reaction mixture was concentrated under vacuum. The obtained solid was dissolved in water (10 mL) and washed with MTBE (10 mL), the water layer was lyophilized to afford 3 afford 3-(4-((4-amino-3,3-dimethylbutyl)(pentyl)amino)-1-oxoisoindolin-2-yl)piperidine-2,6-dione, HCl 123 (70 mg, 0.145 mmol, 96% yield) as a pale brown solid.

¹H-NMR (400 MHz, DMSO-d₆): δ 11.00 (s, 1H), 7.87 (s, 3H), 7.16-7.43 (m, 3H), 5.11 (s, 1H), 4.33-4.35 (m, 2H), 3.22 (m, 4H), 2.90 (m, 1H), 2.63 (m, 3H), 2.50 (m, 3H), 2.10 (m, 1H), 1.43 (m, 3H), 1.24 (m, 4H), 0.94 (m, 5H), 0.83 (m, 3H)

LCMS: 429.3 (M+H). Method: Column: X-BRIDGE C8 (50×4.6) 3.5 μm, Mobile phase: A: 0.1% TFA in H₂O, B: 0.1% TFA in Acetonitrile, Flow Rate: 1.5 ml/min.

HPLC: Method: Mobile phase: A: 10 mM Ammonium bicarbonate in water, Mobile phase: B: Acetonitrile, Flow: 1.0 mL/min, Column:X-Bridge C8(50×4.6) mm, 3.5 μm, RT: 5.333 min, Area: 96.027%.

### Hydrochloride Salt Form of Compound 124

### 3-(4-((4-amino-3,3-dimethylbutyl)(4-aminobutyl)amino)-1-oxoisoindolin-2-yl)piperidine-2,6-dione dihydrochloride

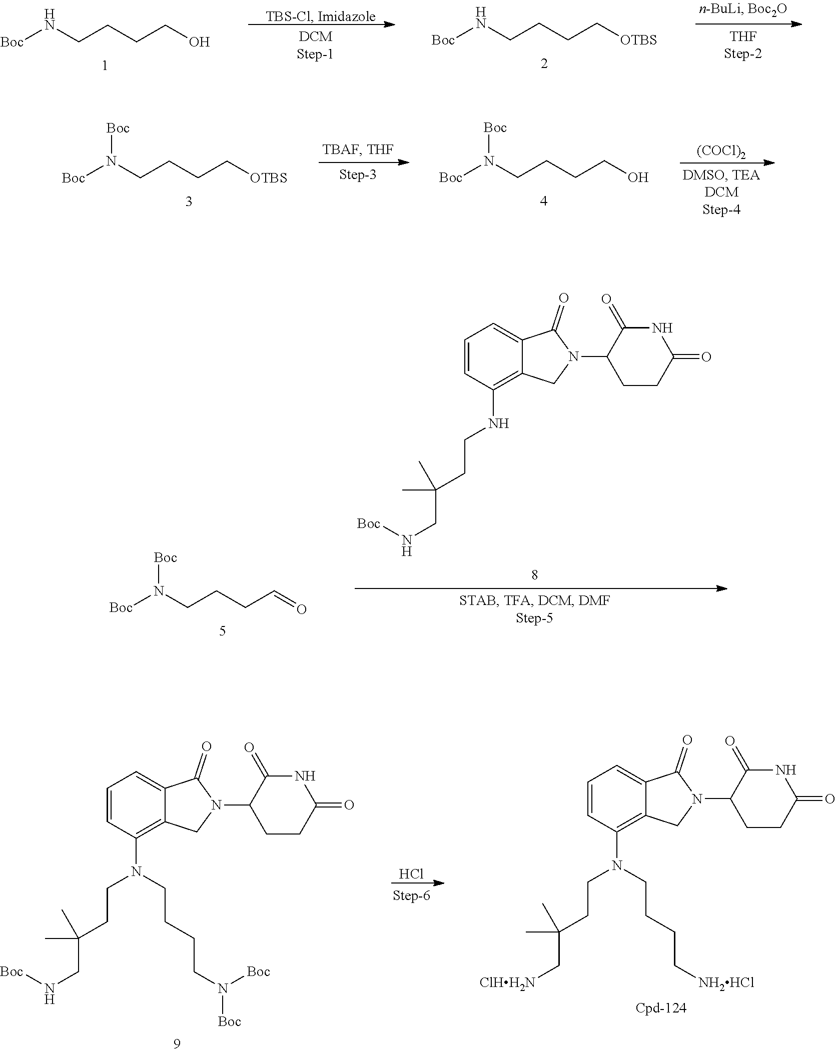

### Step-1: tert-butyl (4-((tert-butyldimethylsilyl)oxy)butyl)carbamate

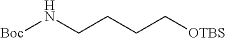 [SMILES: CC(C)(C)OC(=O)NCCCCO[Si](C)(C)C(C)(C)C]

To a solution of tert-butyl (4-hydroxybutyl)carbamate (5.0 g, 26.4 mmol, 1.0 equiv) in dichloromethane (100 ml) was added imidazole (4.5 g, 66.0 mmol, 2.5 equiv) and TBS-Cl (4.78 g, 31.7 mmol, 1.5 equiv) at 0° C. and was allowed to stir at room temperature for 16 hours. Upon completion of the reaction (as confirmed by TLC, 20% EtOAc/pet ether, R_{f}˜0.5, KMnO₄). The reaction mixture was quenched with H₂O (50 ml) and extracted with dichloromethane (1×50 mL). The organic layer was dried over Na₂SO₄ and concentrated under vacuum to give crude compound. The crude compound was purified by chromatography (column size: Biotage R snap cartridge, KP-Sil, 100 g, 230-400 silica gel) using 10-15% EtOAc in pet-ether to afford tert-butyl (4-((tert-butyldimethylsilyl)oxy)butyl)carbamate (8.0 g, 26.3 mmol, 99% yield) as a colorless liquid.

¹H-NMR (400 MHz, DMSO-d6): δ 6.77 (s, 1H), 3.56 (t, 2H), 2.90 (t, 2H), 1.16-1.20 (m, 4H), 1.07 (s, 9H), 0.86 (s, 9H), 0.03 (s, 6H).

### Step-2: tert-butyl (tert-butoxycarbonyl)(4-((tert-butyldimethylsilyl)oxy)butyl)carbamate

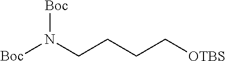 [SMILES: CC(C)(C)OC(=O)N(CCCCO[Si](C)(C)C(C)(C)C)C(=O)OC(C)(C)C]

n-BuLi (2.5 M soln. in hexane, 12.65 ml, 31.6 mmol, 1.2 equiv) was added to a stirred solution of tert-butyl (4-((tert-butyldimethylsilyl)oxy)butyl)carbamate 2 (8.0 g, 26.4 mmol, 1.0 equiv) in THF (80 ml)) at 0° C. and stirred for 15 min at 0° C. A solution of (Boc)₂O (7.34 ml, 31.6 mmol, 1.2 equiv) in THF (20 ml) was added to the reaction mixture at same temperature. The resulting reaction mixture was warmed to room temperature and stirred for 1 h. Upon completion of the reaction (as confirmed by TLC analysis, 10% EtOAc/pet ether, R_{f}˜0.7, KMnO₄), the reaction mixture was quenched with water (50 ml) and extracted with dichloromethane (2×100 ml). The combined organic layer was dried over Na₂SO₄ and concentrated under reduced pressure to afford crude of tert-butyl (tert-butoxycarbonyl)(4-((tert-butyldimethylsilyl)oxy)butyl)carbamate 3 (11.0 g, 27.0 mmol, 102% yield) as a pale yellow liquid, which was used further without purification.

¹H-NMR (400 MHz, DMSO-d6): δ 3.58 (t, 2H), 3.48 (t, 2H), 1.16-1.20 (m, 4H), 1.07 (s, 18H), 0.86 (s, 9H), 0.03 (s, 6H).

### Step-3: tert-butyl (tert-butoxycarbonyl)(4-hydroxybutyl)carbamate

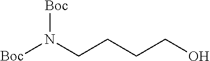 [SMILES: CC(C)(C)OC(=O)N(CCCCO)C(=O)OC(C)(C)C]

To a stirred solution of the tert-butyl (tert-butoxycarbonyl)(4-((tert-butyldimethylsilyl) oxy)butyl)carbamate 3 (10 g, 24.77 mmol, 1.0 equiv) in THF (160 ml) at room temperature, was added TBAF (1M soln. in THF, 24.77 ml, 24.77 mmol, 1.5 equiv) and stirred at same temperature for over night. Upon completion of the reaction (as confirmed by TLC analysis, 20% EtOAc/pet ether, R_{f}˜0.2, KMnO₄). The reaction mixture was quenched with water (50 ml) and extracted with DCM (2×50 ml). The combined organic layer was dried over Na₂SO₄ and concentrated under reduced pressure to afford the crude as a dark yellow liquid. The crude compound was purified by Isolera chromatography (Biotage R snap cartridge, KP-Sil, 100 g, 230-400 silica gel) using 15-20% EtOAc in pet-ether to afford tert-butyl (tert-butoxycarbonyl)(4-hydroxybutyl)carbamate 4 (6 g, 20.53 mmol, 83% yield) as yellow liquid.

¹H-NMR (400 MHz, DMSO-d₆): δ 4.40 (t, J=6.80 Hz, 1H), 3.41-3.48 (m, 2H), 3.33-3.39 (m, 2H), 1.44 (m, 4H), 1.27 (s, 18H)

### Step-4: tert-butyl (tert-butoxycarbonyl)(4-oxobutyl)carbamate

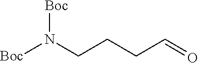 [SMILES: CC(C)(C)OC(=O)N(CCCC=O)C(=O)OC(C)(C)C]

A solution of oxalyl chloride (1.8 ml, 20.73 mmol, 2.0 equiv) and CH₂Cl₂ (30 ml) was added DMSO (2.95 ml, 41.5 mmol, 4.0 equiv) in CH₂Cl₂ (5 ml) at −78° C. and stirred for 15 minutes at same temperature. To this mixture, a solution of tert-butyl (tert-butoxycarbonyl)(4-hydroxybutyl)carbamate 4 (3 g, 10.37 mmol, 1.0 equiv) in CH₂Cl₂ (10 ml) was added at −78° C. After stirring the reaction mixture for 30 minutes at −78° C., triethylamine (9 ml, 62.2 mmol, 6.0 equiv in drops at −78° C. Then, the reaction mixture was slowly warmed to 0° C. and stirred for 30 min. Upon completion of the reaction (as confirmed by TLC analysis, 45% EtOAc/pet ether, R_{f}˜0.6, KMnO₄), the reaction mixture was diluted with DCM (20 mL) and washed with 10% aq citric acid solution (1×100 mL), and water (1×100 mL), dried over Na₂SO₄ and concentrated under reduced pressure (below 30° C.). The crude was codistilled with toluene to afford tert-butyl (tert-butoxycarbonyl)(4-oxobutyl)carbamate 5 (3.2 g, 11.14 mmol, 107% yield) as yellow liquid, which was used further without purification.

¹H-NMR (400 MHz, DMSO-d6): δ 9.65 (s, 1H), 3.48 (t, J=9.20 Hz, 2H), 2.41-2.50 (m, 2H), 1.70-2.30 (m, 2H), 1.44 (s, 18H),

### Step-5: tert-butyl(tert-butoxycarbonyl)(4-((4-((tert-butoxycarbonyl)amino)-3,3-dimethylbutyl)(2-(2,6-dioxopiperidin-3-yl)-1-oxoisoindolin-4-yl) amino) butyl)carbamate

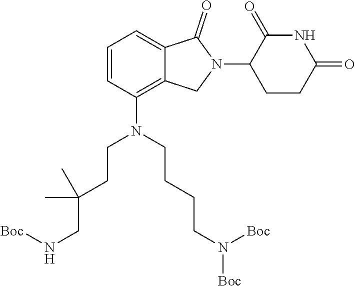 [SMILES: CC(C)(CCN(CCCCN(C(=O)OC(C)(C)C)C(=O)OC(C)(C)C)c1cccc2c1CN(C1CCC(=O)NC1=O)C2=O)CNC(=O)OC(C)(C)C]

To a stirred mixture of tert-butyl (4-((2-(2,6-dioxopiperidin-3-yl)-1-oxoisoindolin-4-yl)amino)-2,2-dimethylbutyl)carbamate 8 (0.3 g, 0.654 mmol, 1.0 equiv) and tert-butyl (tert-butoxycarbonyl)(4-oxobutyl)carbamate 5 (0.376 g, 1.308 mmol, 2.0 equiv) in CH₂Cl₂ (5 ml) and DMF (5.00 ml) was added trifluoroacetic acid (0.202 ml, 2.62 mmol, 4.0 equiv) and stirred for 10 min at 0° C. To this mixture, sodium triacetoxyborohydride (0.555 g, 2.62 mmol, 4.0 equiv) was added in portions at 0° C. and the resulted colourless cloudy mass was stirred for 16 h at room temperature. Upon completion of the reaction (as confirmed by TLC analysis, 100% EtOAc, Rf˜0.7), the reaction mixture was quenched with ice cold water (20 ml) and extracted with DCM (2×50 ml). The combined organic layer was dried over Na₂SO₄ and concentrated under vacuum to afford crude compound. The crude compound was purified by prep-HPLC purification (Column: X-Bridge-c18 19.1×250, Mobile phase: 0.1% FA in Water/ACN, FLOW rate: 15 ml/min), to afford tert-butyl (tert-butoxycarbonyl)(4-((4-((tert-butoxycarbonyl)amino)-3,3-dimethylbutyl)(2-(2,6-dioxopiperidin-3-yl)-1-oxoisoindolin-4-yl)amino)butyl)carbamate 9 (75 mg, 0.100 mmol, 15.28% yield) as a white solid.

¹H-NMR (400 MHz, DMSO-d6): δ 10.95 (s, 1H), 7.35-7.39 (m, 1H), 7.19-7.21 (m, 1H), 7.05-7.07 (m, 1H), 6.79-6.81 (m, 1H), 5.10-5.14 (m, 1H), 4.26-4.41 (m, 2H), 3.43-3.46 (m, 2H), 3.17-3.19 (m, 4H), 2.89-2.93 (m, 1H), 2.68-2.75 (m, 2H), 2.00-2.02 (m, 2H), 1.29-1.49 (m, 16H), 1.24 (s, 18H), 0.80 (s, 6H).

LCMS: 730.4 (M+H). Method: Atlantis dC18 (50×4.6 mm) 5 μm, Mobile phase: A: 0.1% FA in H₂O, Mobile phase: B: Acetonitrile, Flow Rate: 1.5 ml/min.

HPLC: Method: Mobile Phase A: 0.1% TFA in water, Mobile Phase B: Acetonitrile, Flow rate: 2.0 ml/min. Column: X-Bridge C8(50×4.6) mm, 3.5 μm. RT: 5.166 min, Area:97.332%

### Step-6: 3-(4-((4-amino-3,3-dimethylbutyl)(4-aminobutyl)amino)-1-oxoisoindolin-2-yl)piperidine-2,6-dione dihydrochloride

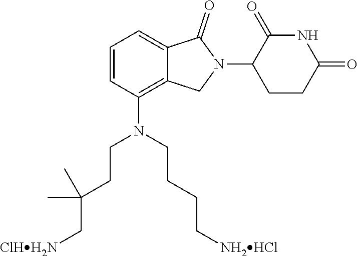

To a stirred solution of tert-butyl (tert-butoxycarbonyl)(4-((4-((tert-butoxycarbonyl)amino)-3,3-dimethylbutyl)(2-(2,6-dioxopiperidin-3-yl)-1-oxoisoindolin-4-yl)amino)butyl)carbamate 9 (70 mg, 0.096 mmol, 1.0 equiv) in CH₂Cl₂ (3 ml) was added HCl (4M soln. in dioxane, 5 ml) at 0° C. and then stirred at room temperature for 2 h. The progress of the reaction was monitored by LCMS. Reaction mixture was concentrated under vacuum at below 40° C. to afford pale yellow solid. The obtained solid was dissolved in water (10 ml) and washed with MTBE (2×10 ml), the water layer was lyophilized to afford 3-(4-((4-amino-3,3-dimethylbutyl)(4-aminobutyl)amino)-1-oxoisoindolin-2-yl)piperidine-2,6-dione, 2HCl 124 (40 mg, 0.077 mmol, 80% yield) as a off-white solid.

¹H-NMR (400 MHz, DMSO-d6): δ 11.00 (s, 1H), 7.93 (s, 6H), 7.34-7.44 (m, 1H), 7.16-7.28 (m, 2H), 5.11-5.16 (m, 1H), 4.30-4.45 (m, 2H), 3.23 (m, 4H), 2.90-2.98 (m, 1H), 2.76-2.77 (m, 2H), 2.64-2.68 (m, 3H), 2.02-2.05 (m, 1H), 1.52 (m, 7H), 0.95-0.96 (m, 6H),

LCMS: 430.2 (M+H). Method:Column: Atlantis dC18 (50×4.6) mm, 5 μm, Mobile phase: A: 0.1% FA in H₂O, Mobile phase: B: Acetonitrile, Flow Rate: 1.5 ml/min.

HPLC: Method: Mobile Phase: 0.1% TFA in water, Mobile Phase B: Acetonitrile, Flow rate: 2.0 ml/min. Column: X-Bridge C8(50×4.6) mm, 3.5 μm. RT: 1.407 min, Area:96.954%.

### Hydrochloride Salt Form of Compound 125

### 3-(4-((5-amino-3,3-dimethylpentyl)(4-aminobutyl)amino)-1-oxoisoindolin-2-yl)piperidine-2,6-dione dihydrochloride

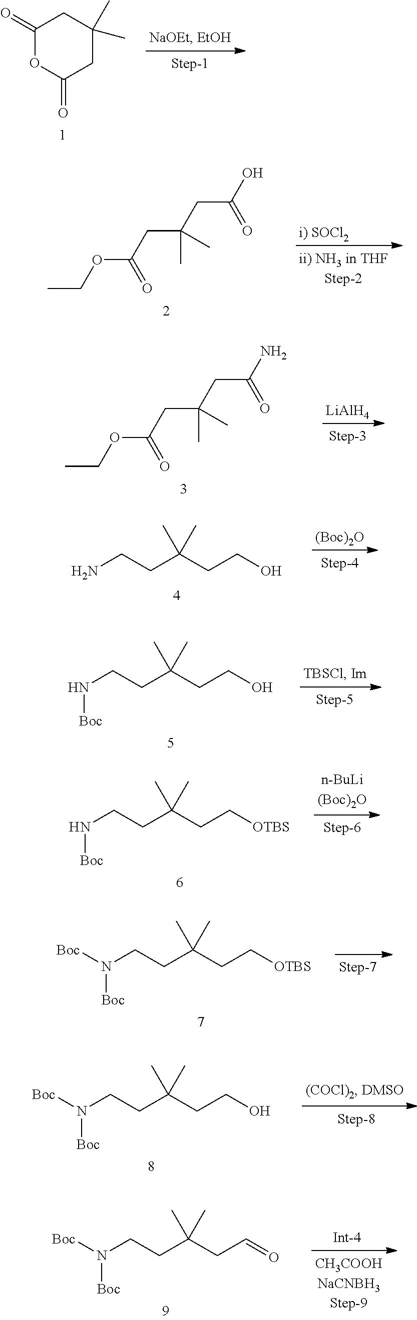

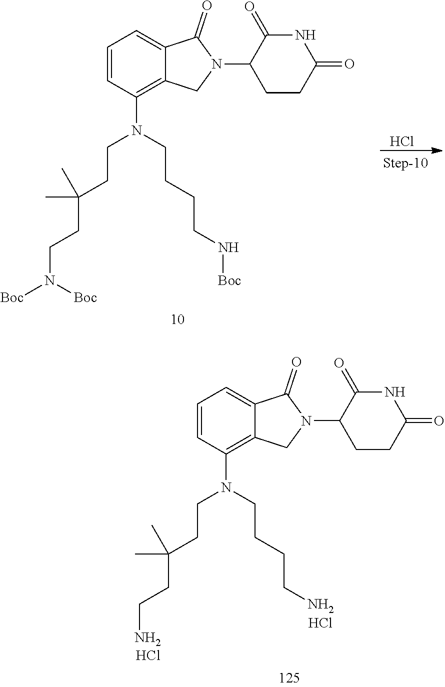 [SMILES: CC(C)(CCN(C(=O)OC(C)(C)C)C(=O)OC(C)(C)C)CCN(CCCCNC(=O)OC(C)(C)C)c1cccc2c1CN(C1CCC(=O)NC1=O)C2=O ; Cl ; CC(C)(CCN)CCN(CCCCN)c1cccc2c1CN(C1CCC(=O)NC1=O)C2=O ; Cl ; Cl]

### Step-1: 5-ethoxy-3,3-dimethyl-5-oxopentanoic acid

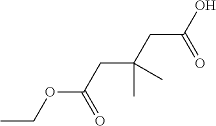

To an ice cold solution of 4,4-dimethyldihydro-2H-pyran-2,6(3H)-dione 1 (10 g, 70.3 mmol, 1 equiv) in ethanol (50 mL, 5 vol), was added sodium ethoxide (0.479 g, 7.03 mmol, 0.1 equiv) dropwise and stirred for 16 h at 85° C. Upon completion of the reaction (as confirmed by LCMS analysis), the reaction mixture was distilled under reduced pressure. The resulted residue was poured into ice cold solution of sat. NaHCO₃ solution (200 mL) and washed with EtOAc (2×50 mL). The separated aqueous layer was further acidified with aq. HCl (6N, 40 mL, pH-2) and extracted with EtOAc (2×50 mL). The combined organic extracts were washed with brine (200 mL), dried over Na₂SO₄, filtered and concentrated to afford 5-ethoxy-3,3-dimethyl-5-oxopentanoic acid 2 (6 g, 31.9 mmol, 45.3% yield) as colorless liquid.

¹H-NMR (400 MHz, DMSO-d6): δ 12.03 (s, 1H), 4.04 (q, J=7.20 Hz, 2H), 2.37 (s, 2H), 2.29 (d, J=7.20 Hz, 2H), 1.18 (t, J=7.20 Hz, 3H), 1.05 (s, 6H).

### Step-2: ethyl 5-amino-3,3-dimethyl-5-oxopentanoate

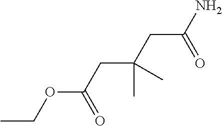

To an ice cold solution of 5-ethoxy-3,3-dimethyl-5-oxopentanoic acid 2 (6 g, 31.9 mmol, 1 equiv) in DCM (60 mL, 10 vol) was added thionyl chloride (15 ml, 206 mmol, 6 equiv) dropwise followed by catalytic amount of DMF (2 drops) and stirred for 3.5 hours at room temperature. Upon completion of the reaction (as confirmed by TLC analysis, 30% EtOAc in pet ether, R_{f}˜0.8, as methyl ester), the reaction mixture was distilled and dried under reduced pressure. The resulted acid chloride mixture was dissolved in THF (30 mL) and added dropwise to a pre-cooled solution of ammonia (0.5 M in THF, 90 ml, 4159 mmol) at −78° C. The reaction mixture was warmed to room temperature and stirred for 30 minutes. Upon completion of the reaction (as confirmed by TLC analysis, 30% EtOAc in pet ether, R_{f}˜0.2), the reaction mixture was concentrated and resulted residue was poured into ice cold solution of sat. NaHCO₃ solution (150 mL) and extracted with EtOAc (2×50 mL). The combined organic layer was washed with brine (150 mL), dried over Na₂SO₄, filtered and concentrated to afford ethyl 5-amino-3,3-dimethyl-5-oxopentanoate 3 (4.4 g, 159 mmol, 50% yield) as yellow liquid.

¹H-NMR (400 MHz, DMSO-d₆): δ 7.21 (s, 1H), 6.73 (s, 1H), 4.04 (q, J=7.20 Hz, 2H), 2.36 (s, 2H), 2.08 (s, 2H), 1.18 (t, J=7.20 Hz, 3H), 1.03 (s, 6H).

LCMS: 188.1 (M+H), Rt (min): 1.694, Area %: 99.678.

### Step-3: 5-amino-3,3-dimethylpentan-1-ol

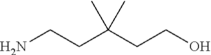

A solution of ethyl 5-amino-3,3-dimethyl-5-oxopentanoate 3 (4.4 g, 23.50 mmol, 1 equiv) in THF (15 mL, 8 vol) was treated with LAH (2M in THF, 23.50 ml, 47.0 mmol, 2 equiv) dropwise at 0° C. over a period of 5 minutes. Once the addition was complete, the reaction mixture was warmed to room temperature, then slowly heated to 65° C. and stirred for 2 hours. Upon completion of the reaction (as confirmed by TLC analysis, EtOAc, R_{f}˜0.2), the reaction mixture was cooled to 0° C. and slowly quenched with saturated Na₂SO₄ solution (60 ml), precipitated salts were removed by filtration through Celite pad and washed with EtOAc (50 mL). The combined filtrate was dried over Na₂SO₄, filtered and concentrated to afford 5-amino-3,3-dimethylpentan-1-ol 4 (2 g, 13.30 mmol, 56.6% yield) as brown liquid.

¹H-NMR (400 MHz, CDCl₃): δ 3.70 (t, J=7.60 Hz, 3H), 2.73 (q, J=8.00 Hz, 2H), 1.54 (t, J=7.60 Hz, 2H), 1.44-1.40 (m, 2H), 1.02 (s, 1H), 0.93 (s, 9H).

LCMS: 132.2 (M+H), Rt (min): 0.603, Area %: 87.229.

### Step-4: tert-butyl (5-hydroxy-3,3-dimethylpentyl)carbamate

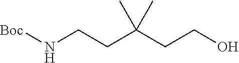 [SMILES: CC(C)(CCO)CCNC(=O)OC(C)(C)C]

To a stirred suspension of 5-amino-3,3-dimethylpentan-1-ol 4 (2 g, 15.24 mmol, 1 equiv) and sodium bicarbonate (2.56 g, 30.5 mmol, 2 equiv) in mixture of THF (30 mL, 15 vol) and water (2 mL, 1 vol) was added Boc-anhydride (3.54 ml, 15.24 mmol, 1 equiv) in dropwise at room temperature. The resulted reaction mixture was stirred for 16 h at room temperature. Upon completion of the reaction (as confirmed by LCMS analysis), anhyd. Na₂SO₄ was added to the reaction mixture, filtered and washed with EtOAc (20 mL). The resulted filtrate was concentrated under reduced pressure to give crude product as yellow liquid. The crude product was purified by column chromatography (Biotage R snap cartridge, KP-Sil, 100 g, 100-200 silica gel) using 20-25% EtOAc in pet ether to afford tert-butyl (5-hydroxy-3,3-dimethylpentyl)carbamate 5 (1.7 g, 7.33 mmol, 48.1% yield) as yellow liquid.

¹H-NMR (400 MHz, DMSO-d₆): δ 6.70 (s, 1H), 3.43 (d, J=4.40 Hz, 2H), 2.94-2.87 (m, 2H), 1.44-1.20 (m, 16H), 0.78 (s, 6H).

LCMS: 132.3 (M-Boc), Rt (min): 2.14, Area %: 99.812.

### Step-5: tert-butyl (5-((tert-butyldimethylsilyl)oxy)-3,3-dimethylpentyl)carbamate

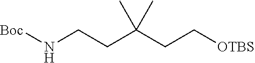 [SMILES: CC(C)(CCNC(=O)OC(C)(C)C)CCO[Si](C)(C)C(C)(C)C]

To a solution of tert-butyl (5-hydroxy-3,3-dimethylpentyl)carbamate 5 (1.7 g, 7.35 mmol, 1.0 equiv) and imidazole (1.251 g, 18.37 mmol, 2.5 equiv) in DCM (20 ml) was added TBS-Cl (1.329 g, 8.82 mmol, 1.2 equiv) at 0° C. and was allowed to stir at room temperature for 13 h. Upon completion of the reaction (as confirmed by TLC, 30% EtOAc in pet ether, R_{f}˜0.9, KMnO₄). The reaction was quenched with water (50 ml) and extracted with DCM (1×30 mL). The combined organic layer was dried over Na₂SO₄ and concentrated under reduced pressure to afford tert-butyl (5-((tert-butyldimethylsilyl)oxy)-3,3-dimethylpentyl)carbamate 6 (2.5 g, 7.22 mmol, 98% yield) as yellow liquid.

¹H-NMR (400 MHz, DMSO-d6): δ 6.69 (t, J=5.20 Hz, 1H), 3.63 (t, J=7.60 Hz, 2H), 2.90 (q, J=5.60 Hz, 2H), 1.40 (s, 10H), 1.37 (s, 2H), 0.85 (t, J=3.20 Hz, 14H), 0.03 (q, J=2.80 Hz, 6H).

LCMS: 246.2 (M-Boc), Rt (min): 3.581, Area %: 99.821.

### Step-6: tert-butyl (tert-butoxycarbonyl)(5-((tert-butyldimethylsilyl)oxy)-3,3-dimethylpentyl)carbamate

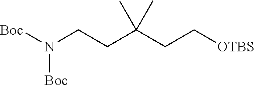 [SMILES: CC(C)(CCO[Si](C)(C)C(C)(C)C)CCN(C(=O)OC(C)(C)C)C(=O)OC(C)(C)C]

To a solution of tert-butyl (5-((tert-butyldimethylsilyl)oxy)-3,3-dimethylpentyl)carbamate 6 (2.5 g, 7.23 mmol) in THF (25 mL, 10 vol) was added n-BuLi (2.5 M in hexane, 3.47 ml, 8.68 mmol, 1.2 equiv) at 0° C. The reaction mixture was stirred for 15 min at 0° C. A solution of (Boc)₂O (1.316 ml, 5.67 mmol, 1.2 equiv) in THF (10 mL) was added to the reaction mixture at same temperature. The resulting reaction mixture was warmed to room temperature and stirred for 1 hour. Upon completion of the reaction (as confirmed by TLC analysis, 5% EtOAc in pet ether, R_{f}˜0.8, KMnO₄). The reaction mixture was quenched with water (50 ml) and extracted with DCM (2×30 ml). The combined organic layer was dried over Na₂SO₄ and concentrated under reduced pressure to afford tert-butyl (tert-butoxycarbonyl)(5-((tert-butyldimethylsilyl)oxy)-3,3-dimethylpentyl)carbamate 7 (3.5 g, 7.75 mmol, 107% yield) as yellow liquid.

¹H-NMR (400 MHz, DMSO-d6): δ 1.47 (s, 9H), 1.44-1.41 (m, 4H), 0.91 (s, 4H), 0.86 (t, J=4.40 Hz, 6H), 0.03 (d, J=4.80 Hz, 3H).

LCMS: 246.3 (M-200), Rt (min): 4.602, Area %: 98.718.

### Step-7: tert-butyl (tert-butoxycarbonyl)(5-hydroxy-3,3-dimethylpentyl)carbamate

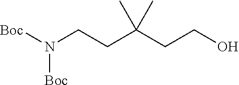 [SMILES: CC(C)(CCO)CCN(C(=O)OC(C)(C)C)C(=O)OC(C)(C)C]

To a solution of tert-butyl-(tert-butoxycarbonyl)(5-((tert-butyldimethylsilyl)oxy)-3,3-dimethylpentyl)carbamate 7 (3.5 g, 7.85 mmol) in THF (35 mL, 10 vol) was added TBAF (11.78 ml, 1M soln. in THF, 11.78 mmol, 1.5 equiv) and stirred for 16 h at room temperature. Upon completion of the reaction (as confirmed by TLC analysis, 30% EtOAc in pet ether, R_{f}˜0.2, KMnO₄). The reaction mixture was quenched with water (100 ml) and extracted with EtOAc (2×30 ml). The combined organic layer was dried over Na₂SO₄ and concentrated under reduced pressure to afford the crude as yellow liquid. The crude compound was purified by column chromatography (Biotage R snap cartridge, KP-Sil, 50 g, 60-120 silica gel) using 12-15% EtOAc in pet-ether to afford tert-butyl (tert-butoxycarbonyl)(5-hydroxy-3,3-dimethylpentyl)carbamate 8 (1.4 g, 4.22 mmol, 53.8% yield) as pale yellow liquid.

¹H-NMR (400 MHz, DMSO-d6): δ 4.26 (t, J=4.80 Hz, 1H), 3.50-3.44 (m, 4H), 1.45 (s, 16H), 1.38 (q, J=7.60 Hz, 5H), 0.88 (s, 6H).

### Step-8: tert-butyl (tert-butoxycarbonyl)(3,3-dimethyl-5-oxopentyl)carbamate

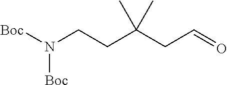 [SMILES: CC(C)(CC=O)CCN(C(=O)OC(C)(C)C)C(=O)OC(C)(C)C]

A solution of oxalyl chloride 8 (0.317 ml, 3.62 mmol, 1.2 equiv) in DCM (10 mL) was added dropwise to a solution of DMSO (0.514 ml, 7.24 mmol, 2.4 equiv) at −78° C. under nitrogen atmosphere and stirred for 15 minutes at the same temperature. To this, a solution of tert-butyl (tert-butoxycarbonyl)(5-hydroxy-3,3-dimethylpentyl)carbamate (1 g, 3.02 mmol, 1 equiv) in DCM (10 mL) was added dropwise over a period of 5 minutes. After stirring the reaction mixture for 30 minutes at −78° C., was added TEA (2.103 ml, 15.09 mmol, 5 equiv) in drops at −78° C. Once addition was complete, the reaction mixture was slowly warmed to 0° C. Upon completion of the reaction (as confirmed by TLC analysis, 30% EtOAc in pet ether, R_{f}˜0.8, KMnO₄), the reaction mixture was diluted with DCM (10 mL) and washed with 10% aq citric acid solution (50 mL), water (50 mL) and brine (50 mL), dried over Na₂SO₄, filtered and concentrated under vacuum below 30° C. to afford tert-butyl (tert-butoxycarbonyl)(3,3-dimethyl-5-oxopentyl)carbamate 9 (930 mg, 2.82 mmol, 94% yield) as yellow liquid which was used further without purification.

¹H-NMR (400 MHz, DMSO-d6): δ 9.76 (q, J=2.40 Hz, 1H), 3.49-3.44 (m, 2H), 2.29 (d, J=2.80 Hz, 1H), 1.52 (d, J=8.00 Hz, 1H), 1.49 (s, 12H), 1.44 (s, 1H), 1.02 (s, 3H), 0.88 (s, 1H).

### Step-9: tert-butyl (tert-butoxycarbonyl)(5-((4-((tert-butoxycarbonyl)amino)butyl)(2-(2,6-dioxopiperidin-3-yl)-1-oxoisoindolin-4-yl)amino)-3,3-dimethylpentyl)carbamate

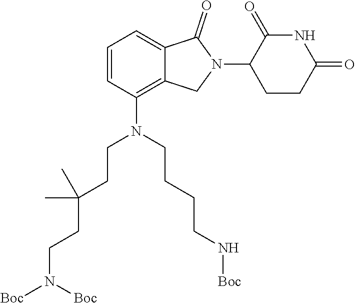 [SMILES: CC(C)(CCN(C(=O)OC(C)(C)C)C(=O)OC(C)(C)C)CCN(CCCCNC(=O)OC(C)(C)C)c1cccc2c1CN(C1CCC(=O)NC1=O)C2=O]

To a solution of tert-butyl (4-((2-(2,6-dioxopiperidin-3-yl)-1-oxoisoindolin-4-yl)amino)butyl)carbamate Int-4 (1 g, 2.323 mmol), tert-butyl (tert-butoxycarbonyl)(3,3-dimethyl-5-oxopentyl)carbamate 9 (930 mg, 2.82 mmol, 1.2 equiv) and TFA (0.716 ml, 9.29 mmol, 4 equiv) in a mixture of Dichloromethane (10 mL, 10 vol) and DMF (10 mL, 10 vol) was added sodium triacetoxyborohydride (1.969 g, 9.29 mmol, 4 equiv) and stirred for 16 h at room temperature. TLC analysis (EtOAc, R_{f}˜0.7) and LCMS indicated product formation and the starting material was not consumed completely. The reaction mixture was diluted with DCM (15 mL) and washed with water (2×30 mL), brine (30 mL), dried over anhy. Na₂SO₄, filtered and concentrated. The resulted residue was purified by prep.HPLC (HCOOH:ACN method) and lyophilized to get tert-butyl (tert-butoxycarbonyl)(5-((4-((tert-butoxycarbonyl)amino)butyl)(2-(2,6-dioxopiperidin-3-yl)-1-oxoisoindolin-4-yl)amino)-3,3-dimethylpentyl)carbamate 10 (70 mg, 0.094 mmol, 4.04% yield) as white solid.

¹H-NMR (400 MHz, DMSO-d6): δ 10.98 (s, 1H), 7.38 (t, J=7.60 Hz, 1H), 7.19 (d, J=7.20 Hz, 1H), 7.08 (d, J=7.60 Hz, 1H), 6.77 (s, 1H), 5.12 (q, J=5.20 Hz, 1H), 4.40 (d, J=16.80 Hz, 1H), 4.29 (d, J=17.20 Hz, 1H), 3.46 (t, J=9.20 Hz, 2H), 3.25 (d, J=28.00 Hz, 4H), 2.93 (t, J=7.20 Hz, 3H), 2.04 (t, J=7.60 Hz, 1H), 1.44 (t, J=8.40 Hz, 20H), 1.37 (d, J=14.00 Hz, 12H), 1.02 (s, 6H).

LC-MS: 744.5 (M+H), Rt (min): 3.010, Area %: 99.774.

### Step-10: 3-(4-((5-amino-3,3-dimethylpentyl)(4-aminobutyl)amino)-1-oxoisoindolin-2-yl)piperidine-2,6-dione dihydrochloride

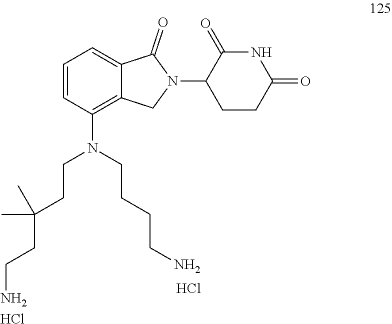 [SMILES: CC(C)(CCN)CCN(CCCCN)c1cccc2c1CN(C1CCC(=O)NC1=O)C2=O ; Cl ; Cl]

To a stirred suspension of tert-butyl (tert-butoxycarbonyl)(5-((4-((tert-butoxycarbonyl)amino)butyl)(2-(2,6-dioxopiperidin-3-yl)-1-oxoisoindolin-4-yl)amino)-3,3-dimethylpentyl)carbamate 10 (70 mg, 0.094 mmol) in dichloromethane (3 mL) was added HCl (4M soln. in dioxane, 1 ml, 4.00 mmol) at 0° C. and then stirred at room temperature for 2 h. Upon completion of the reaction (as confirmed by TLC analysis, EtOAc, Rf˜0.1), the reaction mixture was concentrated under reduced pressure. The obtained solid was dissolved in water (30 mL) and washed with MTBE (2×30 mL), the water layer was lyophilized to afford 3-(4-((5-amino-3,3-dimethylpentyl)(4-aminobutyl)amino)-1-oxoisoindolin-2-yl)piperidine-2,6-dione, 2HCl 125 (12 mg, 0.023 mmol, 24.65% yield) as off-white solid.

¹H-NMR (400 MHz, DMSO-d6): δ 11.01 (s, 1H), 8.00 (s, 6H), 7.54 (s, 2H), 5.17-5.12 (m, 1H), 4.49 (d, J=31.20 Hz, 2H), 3.35 (s, 4H), 2.92 (d, J=12.40 Hz, 1H), 2.75-2.61 (m, 5H), 2.07 (t, J=5.20 Hz, 1H), 1.51 (q, J=8.40 Hz, 6H), 1.39-1.30 (m, 2H), 0.87 (s, 6H).

LCMS: 444.3 (M+H). Method: Mobile Phase A: 0.1% Formic acid in H₂O. Mobile Phase B: ACN. Flow rate: 1.5 ml/min. Column: Atlantis dC18 (50 x4.6) 5 μm. Rt (min): 1.035; Area %: 99.826.

HPLC: Method: Mobile Phase A: 0.1% Formic acid in H₂O. Mobile Phase B: Methanol. Flow rate: 1.0 mL/min. Column: X-Bridge C8(50×4.6) mm, 3.5 μm. Rt (min): 2.679; Area %: 98.409.

### Hydrochloride Salt Form of Compound 126

### 3-(4-((5-amino-4,4-dimethylpentyl)(4-aminobutyl)amino)-1-oxoisoindolin-2-yl)piperidine-2,6-dione dihydrochloride

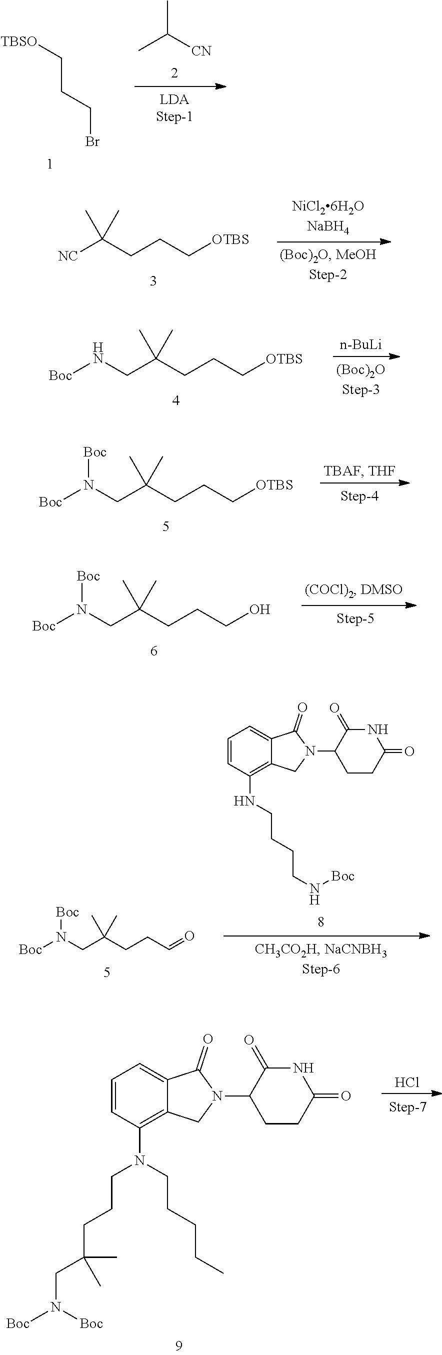

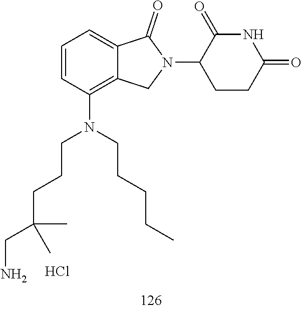 [SMILES: CCCCCN(CCCC(C)(C)CN)c1cccc2c1CN(C1CCC(=O)NC1=O)C2=O ; Cl]

Step-1: 5-((tert-butyldimethylsilyl)oxy)-2,2-dimethylpentanenitrile

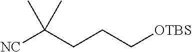 [SMILES: CC(C)(C#N)CCCO[Si](C)(C)C(C)(C)C]

To a solution of LDA (2M in THF/Heptane/Ethyl benzene, 5.92 ml, 11.85 mmol, 1.5 equiv) in THF (10 ml) was cooled to −78° C. and added isobutyronitrile 2 (0.764 g, 11.06 mmol, 1.4 equiv) over 5 minutes. The reaction mixture was allowed to warm to −40° C. and stirred for 30 min. and again cooled to −78° C. Then (3-bromopropoxy)(tert-butyl)dimethylsilane 1 (2.0 g, 7.90 mmol, 1.0 equiv) was added dropwise to the reaction mixture and was allowed to warm to 0° C. and stirred for 1 h. Upon completion of the reaction (as confirmed by TLC and UPLC analysis, 10% EtOAc/pet ether, Rf˜0.6, KMnO₀₄), the reaction mixture was quenched with 1 N HCl (1×10 ml) and extracted with MTBE (2×50 ml). The combined organic layer was washed saturated NaHCO₃ (1×20 ml), brine (1×20 ml) and dried over Na₂SO₄ and concentrated under reduced pressure to afford 5-((tert-butyldimethylsilyl)oxy)-2,2-dimethylpentanenitrile 3 (2.2 g, 9.11 mmol, 115% yield) as a pale yellow liquid, which was used further without purification.

¹H-NMR (400 MHz, DMSO-d₆): δ 3.66-3.69 (m, 2H), 1.69-1.73 (m, 2H), 1.59-1.63 (m, 2H), 1.38 (s, 6H), 0.91 (s, 9H), 0.08 (s, 6H)

### Step-2: tert-butyl (5-((tert-butyldimethylsilyl)oxy)-2,2-dimethylpentyl)carbamate

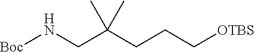 [SMILES: CC(C)(CCCO[Si](C)(C)C(C)(C)C)CNC(=O)OC(C)(C)C]

To a stirred solution of 5-((tert-butyldimethylsilyl)oxy)-2,2-dimethylpentanenitrile 3 (1.0 g, 4.14 mmol, 1.0 equiv) in dry MeOH (30 ml), cooled to 0° C., were added (Boc)₂O (1.923 ml, 8.28 mmol, 2.0 equiv), NiCl₂·6H₂O (0.984 g, 4.14 mmol, 1.0 equiv) and NaBH₄ (1.097 g, 29.0 mmol, 7.0 equiv) was then carefully added in small portions. The reaction was exothermic and effervescent. The resulting reaction mixture was allowed to warm to room temperature and stirred for over night. Upon completion of the reaction (as confirmed by TLC analysis, 5% EtOAc/pet ether, Rf˜0.3, KMnO₄), the reaction mixture was diluted with DCM (20 ml) and filtered through celite bed and washed with DCM (20 ml). The filtrate was washed with saturated NaHCO₃ solution (1×20 ml), the organic layer was dried over Na₂SO₄ and concentrated under reduced pressure to afford crude of tert-butyl (5-((tert-butyldimethylsilyl)oxy)-2,2-dimethylpentyl)carbamate 4 (1.2 g, 3.39 mmol, 82% yield) as a pale yellow liquid, which was used further without purification.

¹H-NMR (400 MHz, DMSO-d6): δ 6.71 (t, J=6.40 Hz, 1H), 3.54 (m, 2H), 2.73-2.76 (m, 2H), 1.44 (s, 9H), 1.22-1.38 (m, 2H), 1.07-1.13 (m, 2H), 0.89 (s, 9H), 0.80 (s, 6H), 0.02 (s, 6H).

### Step-3: tert-butyl(tert-butoxycarbonyl)(5-((tert-butyldimethylsilyl)oxy)-2,2-dimethylpentyl) carbamate

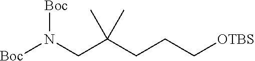 [SMILES: CC(C)(CCCO[Si](C)(C)C(C)(C)C)CN(C(=O)OC(C)(C)C)C(=O)OC(C)(C)C]

To a stirred solution of tert-butyl (5-((tert-butyldimethylsilyl)oxy)-2,2-dimethylpentyl)carbamate 4 (2.5 g, 7.23 mmol, 1.0 equiv) in THF (50 mL) at 0° C., was added n-BuLi (2.5 M soln. in hexane, 3.47 ml, 8.68 mmol, 1.2 equiv). The reaction mixture was stirred for 15 min at 0° C. A solution of (Boc)₂O (2.015 ml, 8.68 mmol, 1.2 equiv) in THF (10 mL) was added to the reaction mixture at same temperature. The resulting reaction mixture was warmed to room temperature and stirred for 1 h. Upon completion of the reaction (as confirmed by TLC analysis, 10% EtOAc/pet ether, R_{f}˜0.7, KMnO₄), the reaction mixture was quenched with water (50 ml) and extracted with DCM (2×100 ml). The combined organic layer was dried over Na₂SO₄ and concentrated under reduced pressure to afford tert-butyl (tert-butoxycarbonyl)(5-((tert-butyldimethylsilyl)oxy)-2,2-dimethylpentyl)carbamate 5 (3.2 g, 6.82 mmol, 94% yield). as a pale yellow liquid, which was used further without purification

¹H-NMR (400 MHz, DMSO-d6): δ 3.59-3.54 (m, 2H), 2.73-2.76 (m, 2H), 1.42 (s, 18H), 1.24-1.36 (m, 2H), 1.07-1.13 (m, 2H), 0.89 (s, 9H), 0.80 (s, 6H), 0.02 (s, 6H).

### Step-4: tert-butyl (tert-butoxycarbonyl)(5-hydroxy-2,2-dimethylpentyl)carbamate

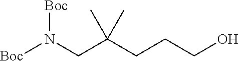 [SMILES: CC(C)(CCCO)CN(C(=O)OC(C)(C)C)C(=O)OC(C)(C)C]

To a stirred solution of the tert-butyl (tert-butoxycarbonyl)(5-((tert-butyldimethylsilyl)oxy)-2,2-dimethylpentyl)carbamate (3.2 g, 7.18 mmol, 1.0 equiv) in THF (60 ml) at room temperature was added TBAF (1M soln. in THF, 10.8 ml, 10.8 mmol, 1.5 equiv) and the resulting solution was stirred at room temperature for over night. Upon completion of the reaction (as confirmed by TLC analysis, 20% EtOAc/pet ether, R_{f}˜0.2, KMnO₄), the reaction mixture was quenched with water (20 ml) and extracted with DCM (2×50 ml). The combined organic layer was dried over Na₂SO₄ and concentrated under reduced pressure to afford the crude product as a dark yellow liquid. The crude compound was purified by Isolera (Biotage R snap cartridge, KP-Sil, 100 g, 100-200 silica gel) using 15-20% EtOAc in pet-ether. The fractions were collected and concentrated under vacuum to afford tert-butyl (tert-butoxycarbonyl)(5-hydroxy-2,2-dimethylpentyl)carbamate 6 (1.6 g, 4.82 mmol, 67.1% yield) as a colorless liquid

¹H-NMR (400 MHz, DMSO-d6): δ 4.35 (t, J=5.20 Hz, 1H), 3.36-3.39 (m, 2H), 3.32-3.35 (m, 2H), 1.47 (s, 18H), 1.36-1.40 (m, 2H), 1.12-1.16 (m, 2H), 0.79 (s, 6H).

### Step-5: tert-butyl (tert-butoxycarbonyl)(5-oxopentyl)carbamate

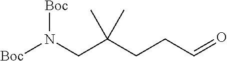 [SMILES: CC(C)(CCC=O)CN(C(=O)OC(C)(C)C)C(=O)OC(C)(C)C]

A solution of oxalyl chloride (0.845 ml, 9.65 mmol, 2.0 equiv) and CH₂Cl₂ (20 ml) was added DMSO (1.370 ml, 19.31 mmol, 4.0 equiv) in CH₂Cl₂ (5 ml) at −78° C. and stirred for 15 minutes at same temperature. To this mixture, a solution of tert-butyl (tert-butoxycarbonyl)(5-hydroxy-2,2-dimethylpentyl)carbamate 6 (1.6 g, 4.83 mmol, 1.0 equiv) in CH₂Cl₂ (5 ml) was added at −78° C. After stirring the reaction mixture for 30 minutes at −78° C., triethylamine (4.04 ml, 29.0 mmol, 6.0 equiv) was added in drops at −78° C. Then, the reaction mixture was slowly warmed to 0° C. and stirred for 30 min. Upon completion of the reaction (as confirmed by TLC analysis, 20% EtOAc/pet ether, R_{f}˜0.6, KMnO₄), the reaction mixture was diluted with DCM (40 mL) and washed with 10% aq citric acid solution (1×40 mL), water (1×40 mL), dried over Na₂SO₄, and concentrated under vacuum (below 30° C.) to afford tert-butyl (tert-butoxycarbonyl)(2,2-dimethyl-5-oxopentyl)carbamate 7 (1.55 g, 3.42 mmol, 70.8% yield) as a pale yellow liquid, which was used further without purification.

¹H-NMR (400 MHz, DMSO-d6): δ 9.68 (s, 1H), 3.39 (s, 2H), 2.42-2.50 (m, 2H), 1.44 (m, 2H), 1.42 (s, 18H), 0.79 (s, 6H)

### Step-6: tert-butyl (tert-butoxycarbonyl)(5-((4-((tert-butoxycarbonyl)amino)butyl)(2-(2,6-dioxopiperidin-3-yl)-1-oxoisoindolin-4-yl)amino)-2,2-dimethylpentyl)carbamate

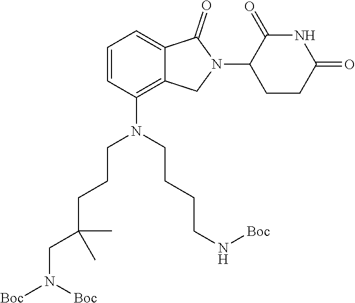 [SMILES: CC(C)(CCCN(CCCCNC(=O)OC(C)(C)C)c1cccc2c1CN(C1CCC(=O)NC1=O)C2=O)CN(C(=O)OC(C)(C)C)C(=O)OC(C)(C)C]

To a stirred mixture of tert-butyl (4-((2-(2,6-dioxopiperidin-3-yl)-1-oxoisoindolin-4-yl)amino)butyl)carbamate 8 (0.5 g, 1.161 mmol, 1.0 equiv) and tert-butyl (tert-butoxycarbonyl)(2,2-dimethyl-5-oxopentyl)carbamate 7 (0.765 g, 2.323 mmol, 2.0 equiv) in CH₂Cl₂ (10 ml) and DMF (10 ml) was added trifluoroacetic acid (0.358 ml, 4.65 mmol, 4.0 equiv) and stirred for 10 min at 0° C. To this mixture, sodium triacetoxyborohydride (0.985 g, 4.65 mmol, 4.0 equiv) was added in portions at 0° C. and the resulted colourless cloudy mass was stirred for 16 h at room temperature. Upon completion of the reaction (as confirmed by TLC analysis, 100% EtOAc, Rf˜0.7), the reaction mixture was quenched with ice cold water (1×20 mL) and extracted with DCM (2×50 mL). The combined organic layer was dried over Na₂SO₄ and concentrated under vacuum to afford crude as a pale yellow liquid. The crude compound was purified by Isolera chromatography (column size: Biotage R snap cartridge, KP-Sil, 100 g, 230-400 silica gel) using 60-70% of ethyl acetate in pet ether. The fractions were collected and concentrated under vacuum to afford tert-butyl (tert-butoxycarbonyl)(5-((4-((tert-butoxycarbonyl)amino)butyl)(2-(2,6-dioxopiperidin-3-yl)-1-oxoisoindolin-4-yl)amino)-2,2-dimethylpentyl)carbamate 9 (0.25 g, 0.335 mmol, 28.9% yield) as a white solid.

¹H-NMR (400 MHz, DMSO-d6): δ 10.98 (s, 1H), 7.35-7.39 (m, 1H), 7.06-7.19 (m, 2H), 6.77-6.79 (m, 1H), 5.09-5.13 (m, 1H), 4.27-4.42 (m, 2H), 3.10-3.21 (m, 4H), 2.89-2.90 (m, 4H), 2.67-2.68 (m, 6H), 1.44 (s, 18H), 1.43 (m, 4H), 1.35 (s, 9H), 1.24 (m, 2H), 0.75 (s, 6H),

LCMS: 744.5 (M+H). Method: Atlantis dC18 (50×4.6 mm) 5 μm, Mobile phase: A: 0.1% FA in H₂O, Mobile phase: B: Acetonitrile, Flow Rate: 1.5 ml/min

HPLC: Method: Mobile Phase A: 0.1% FA in water, Mobile Phase B: Acetonitrile, Flow rate: 2.0 ml/min. Column: X-Bridge C8(50×4.6) mm, 3.5 μm. RT: 6.191 min, Area:96.037%

### Step-7: 3-(4-((5-amino-4,4-dimethylpentyl)(4-aminobutyl)amino)-1-oxoisoindolin-2-yl)piperidine-2,6-dione dihydrochloride

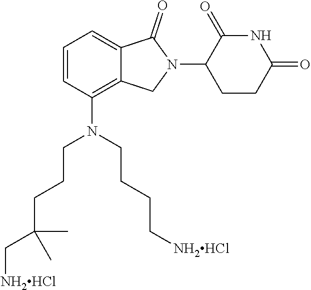

To a stirred solution of tert-butyl (tert-butoxycarbonyl)(5-((4-((tert-butoxycarbonyl)amino)butyl)(2-(2,6-dioxopiperidin-3-yl)-1-oxoisoindolin-4-yl)amino)-2,2-dimethylpentyl)carbamate 9 (0.15 g, 0.202 mmol, 1.0 equiv) in dichloromethane (10 ml) was added HCl (4M soln. in dioxane, 10 ml) at 0° C. and then stirred at room temperature for 2 h. The progress of the reaction was monitored by LCMS. Reaction mixture was concentrated under vacuum at below 40° C., to afford pale yellow solid. The obtained solid was dissolved in water (10 ml) and washed with MTBE (1×10 ml), the water layer was lyophilized to afford 3-(4-((5-amino-4,4-dimethylpentyl)(4-aminobutyl)amino)-1-oxoisoindolin-2-yl)piperidine-2,6-dione, 2HCl (80 mg, 0.148 mmol, 73.4% yield) as a pale yellow solid

¹H-NMR (400 MHz, DMSO-d6): δ 10.99 (s, 1H), 7.94 (s, 6H), 7.43 (m, 1H), 7.25-7.28 (m, 2H), 5.11-5.15 (m, 1H), 4.12-4.50 (m, 2H), 3.20-3.26 (m, 4H), 2.90-2.98 (m, 1H), 2.76-2.89 (m, 2H), 2.64-2.68 (m, 3H), 2.02-2.05 (m, 1H), 1.54 (m, 4H), 1.39 (m, 2H), 1.24-1.26 (m, 3H), 0.93 (s, 6H),

LCMS: 444.4 (M+H). Method: Column: Atlantis dC18 (50×4.6 mm) 5 μm, Mobile phase: A: 0.1% FA in H₂O, Mobile phase: B: Acetonitrile, Flow Rate: 1.5 ml/min.

HPLC: Method: Mobile Phase A: 0.1% TFA in water, Mobile Phase B: Methanol, Flow rate:1.0 ml/min. Column: X-Bridge C8(50×4.6) mm, 3.5 μm. RT: 3.466 min, Area:95.684%.

### Compound 143

### 1-(4-((2-(2,6-dioxopiperidin-3-yl)-1-oxoisoindolin-4-yl)(pentyl)amino)butyl)-3-methylurea

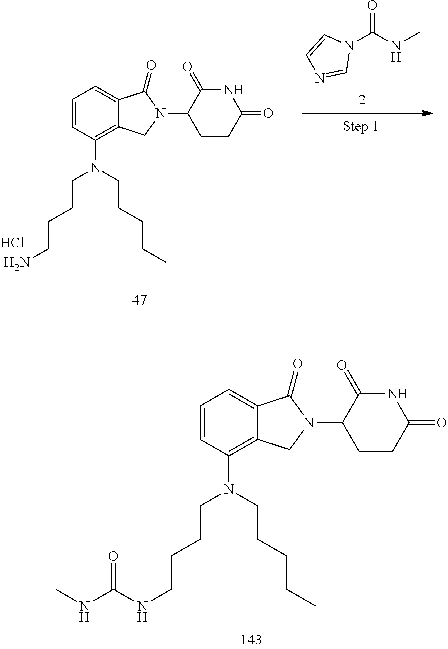

To a solution of 3-(4-((4-aminobutyl)(pentyl)amino)-1-oxoisoindolin-2-yl)piperidine-2,6-dione, HCl (200 mg, 0.499 mmol, 1 equiv) and N-methyl-1H-imidazole-1-carboxamide (62.5 mg, 0.499 mmol, 1 equiv) in DCM (4 ml) was added TEA (0.077 ml, 0.549 mmol, 1.1 equiv) and the resulting reaction mixture was stirred for 12 h at room temperature. Upon completion of the reaction (as confirmed by UPLC), the reaction mixture was concentrated under vacuum to give the crude product which was purified by preparatory HPLC (Column: XBridge C8—150; Method: HCl and ACN, flow rate: 20 ml/min) to give 1-(4-((2-(2,6-dioxopiperidin-3-yl)-1-oxoisoindolin-4-yl)(pentyl)amino)butyl)-3-methylurea (45 mg, 0.098 mmol, 19.56% yield) as white solid.

¹H-NMR (400 MHz, DMSO-d6): δ 11.03 (s, 1H), 7.61 (s, 2H), 5.13 (q, J=4.80 Hz, 1H), 4.74 (s, 2H), 2.96-2.88 (m, 3H), 2.65 (q, J=17.60 Hz, 1H), 2.43 (q, J=4.00 Hz, 1H), 2.08 (t, J=5.60 Hz, 1H), 1.37 (t, J=6.00 Hz, 5H), 1.22 (d, J=2.80 Hz, 5H), 0.81 (t, J=6.80 Hz, 3H).

LCMS: 458.9 (M+H), Method: Mobile phase: A: 0.1% Formic Acid in H₂O B: ACN; Column: Atlantis dC18 (50×4.6) 5μ; Flow Rate: 1.5 ml/min; Rt (min): 1.830, Area %: 99.292.

HPLC: Method: Mobile phase: A: 0.1% FA in water, Mobile phase D: ACN; Column: XBridge C8(50×4.6) mm, 3.5 m, Flow rate:2.0 ml/min; Rt (min): 2.870, Area %: 99.180.

### Compound 148

### N-(4-((2-(2,6-dioxopiperidin-3-yl)-1-oxoisoindolin-4-yl)(pentyl)amino)butyl)butyramide

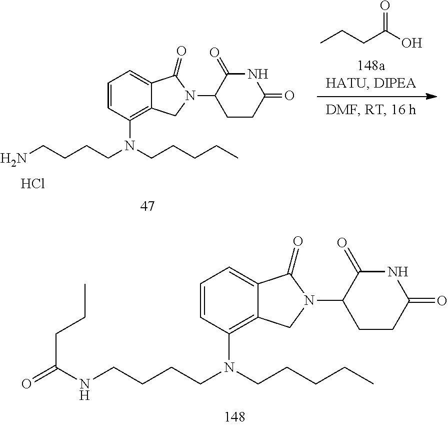

To a stirred solution of butyric acid 148a (0.150 g, 0.375 mmol, 1 equiv) in DMF (2 mL) were added DIPEA (0.262 ml, 1.498 mmol, 4 equiv) and HATU (0.214 g, 0.562 mmol, 1.5 equiv) at 0° C. 3-(4-((4-aminobutyl)(pentyl)amino)-1-oxoisoindolin-2-yl)piperidine-2,6-dione, HCl 47 (0.150 g, 0.375 mmol, 1 equiv) was then added at same temperature and the reaction mixture was stirred at room temperature for 16 h. Upon completion of the reaction (as monitored by LCMS), reaction mixture was purified by prep-HPLC (Instrument: SC-DC-ARD-05-044; FLOW:15 ml/min; Column: X-Select-C18-19×150 mm; Mobile phase combination: 0.1% FA in Water/MeOH). The pure fractions were lyophilized to afford N-(4-((2-(2,6-dioxopiperidin-3-yl)-1-oxoisoindolin-4-yl)(pentyl)amino)butyl)butyramide 148 (37.43 mg, 0.079 mmol, 20.99% yield) as off white solid.

LCMS: 471.2 (M+H). Method: Column: Atlantis dC18 (50×4.6) 5μ, Mobile phase: A: 0.1% Formic Acid in H₂O B: ACN, Flow Rate: 1.5 ml/min. Rt (min): 2.127; Area %—99.68.

HPLC: 98.85%, Rt (min): 3.792. Method: Column: X-Bridge C8(50×4.6) mm, 3.5 μm, Mobile phase: A: 0.1% FA in water, Mobile phase: B: ACN, Flow: 2.0 mL/min.

¹H-NMR (400 MHz, DMSO-d₆): δ 10.98 (s, 1H), 7.71 (d, J=5.20 Hz, 1H), 7.37 (t, J=7.60 Hz, 1H), 7.19 (d, J=7.20 Hz, 1H), 7.07 (d, J=8.00 Hz, 1H), 5.10 (q, J=4.80 Hz, 1H), 4.34 (dd, J=16.80, 43.20 Hz, 2H), 3.17 (t, J=7.20 Hz, 4H), 3.02 (d, J=6.00 Hz, 2H), 2.91 (t, J=12.80 Hz, 1H), 2.68 (s, 1H), 2.62 (s, 1H), 1.99 (t, J=7.20 Hz, 3H), 1.48-1.41 (m, 8H), 1.25 (d, J=3.20 Hz, 4H), 0.86-0.80 (m, 6H).

### Compound 149

### N-(4-((2-(2,6-dioxopiperidin-3-yl)-1-oxoisoindolin-4-yl)(pentyl)amino)butyl)-3-methylbutanamide

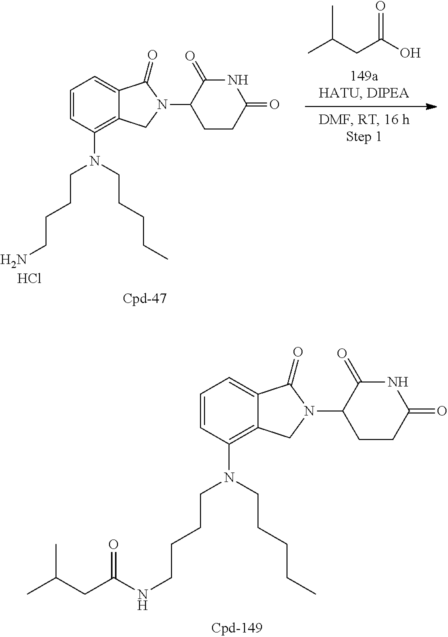

To a stirred solution of 3-methylbutanoic acid 149a (35.1 mg, 0.343 mmol, 1 equiv) in DMF (3 ml) were added HATU (196 mg, 0.515 mmol, 1.5 equiv) and DIPEA (177 mg, 1.373 mmol, 4 equiv) at 0° C., followed by addition of 3-(4-((4-aminobutyl)(pentyl)amino)-1-oxoisoindolin-2-yl)piperidine-2,6-dione, HCl 47 (150 mg, 0.343 mmol, 1 equiv) and the reaction mixture was stirred at room temperature for 16 h. Upon completion of the reaction (as monitored by UPLC), reaction mixture was purified by reverse-phase column chromatography (Grace column: C18 40 μm, 40 g; flow rate: 20 mL/min; 0.1% aqueous HCOOH/ACN mobile phase). The pure fractions were lyophilised to afford N-(4-((2-(2,6-dioxopiperidin-3-yl)-1-oxoisoindolin-4-yl)(pentyl) amino)butyl)-3-methylbutanamide 149 (51.28 mg, 0.106 mmol, 30.8% yield) as off white solid.

¹H-NMR (400 MHz, DMSO-d6): δ 10.98 (s, 1H), 7.73 (d, J=5.20 Hz, 1H), 7.37 (t, J=7.60 Hz, 1H), 7.18 (d, J=7.20 Hz, 1H), 7.07 (d, J=7.60 Hz, 1H), 5.11 (q, J=5.20 Hz, 1H), 4.39 (d, J=16.80 Hz, 1H), 4.28 (d, J=16.80 Hz, 1H), 3.16 (t, J=6.80 Hz, 4H), 3.02 (d, J=5.60 Hz, 2H), 2.92 (s, 1H), 2.62 (s, 1H), 2.01 (t, J=5.60 Hz, 1H), 1.92 (t, J=11.20 Hz, 3H), 1.42 (d, J=7.20 Hz, 6H), 1.25 (s, 4H), 0.84 (t, J=6.00 Hz, 9H).

LCMS: 485.3 (M+H), Method: Mobile phase: A: 0.1% FA in H₂O; Mobile phase: B: ACN; Column: Atlantis dC18 (50×4.6 mm) 5 μm; Column: Atlantis dC18 (50×4.6 mm) 5 μm; Flow Rate: 1.5 ml/min, Rt (min): 2.252, Area %: 99.867.

HPLC: Method: Mobile phase A: 0.1% TFA in water; Mobile phase D: ACN; Column: XBridge C8(50×4.6) mm, 3.5 μm; Flow rate: 2.0 ml/min, Rt (min): 3.324 min, Area %: 99.501.

### Compound 150

### N-(4-((2-(2,6-dioxopiperidin-3-yl)-1-oxoisoindolin-4-yl)(pentyl)amino)butyl)-3,3-dimethylbutanamide

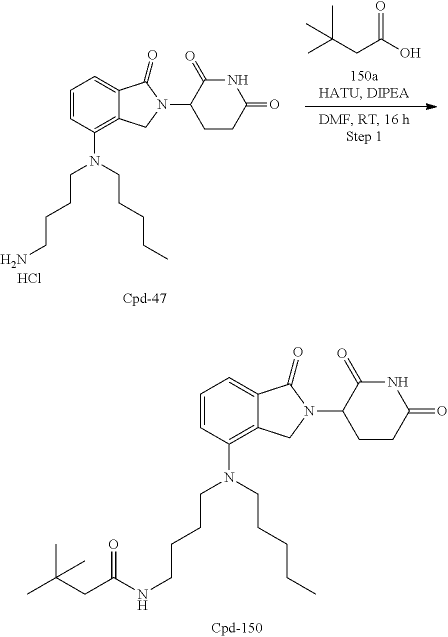

To a stirred solution of 3,3-dimethylbutanoic acid 150a (39.9 mg, 0.343 mmol, 1 equiv) in DMF (3 ml) were added HATU (196 mg, 0.515 mmol, 1.5 equiv) and DIPEA (177 mg, 1.373 mmol, 4 equiv) at 0° C., followed by addition of 3-(4-((4-aminobutyl)(pentyl)amino)-1-oxoisoindolin-2-yl)piperidine-2,6-dione, HCl 47 (150 mg, 0.343 mmol, 1 equiv) and the reaction mixture was stirred at room temperature for 16 h. Upon completion of the reaction (as monitored by UPLC), reaction mixture was purified by reverse-phase column chromatography (Grace column: C18 40 μm, 40 g; flow rate: 20 mL/min; 0.1% aqueous HCOOH/ACN mobile phase). The pure fractions were lyophilised to afford give N-(4-((2-(2,6-dioxopiperidin-3-yl)-1-oxoisoindolin-4-yl)(pentyl)amino) butyl)-3,3-dimethylbutanamide 150 (40.2 mg, 0.080 mmol, 23.28% yield) as off white solid.

¹H-NMR (400 MHz, DMSO-d6): δ 10.98 (s, 1H), 7.69 (d, J=5.20 Hz, 1H), 7.38 (t, J=5.60 Hz, 1H), 7.21 (t, J=6.00 Hz, 1H), 7.11 (s, 1H), 5.14-5.08 (m, 1H), 4.43-4.27 (m, 2H), 3.18 (d, J=6.00 Hz, 4H), 3.01 (d, J=4.80 Hz, 3H), 2.62 (s, 1H), 2.02 (d, J=6.00 Hz, 1H), 1.91 (d, J=5.60 Hz, 2H), 0.92 (d, J=5.20 Hz, 9H), 0.84 (d, J=5.60 Hz, 3H).

LCMS: 499.3 (M+H), Method: Mobile phase: A: 0.1% FA in H₂O; Mobile phase: B: ACN; Column: Atlantis dC18 (50×4.6 mm) 5 μm; Rt (min): 2.340 min, Area %: 99.116.

HPLC: Method: Mobile phase A:0.1% TFA in water; Mobile phase D: ACN; Column: XBridge C8(50×4.6) mm, 3.5 m; Rt (min): 3.570, Area %: 98.021.

### Compound 151

### 2-cyclohexyl-N-(4-((2-(2,6-dioxopiperidin-3-yl)-1-oxoisoindolin-4-yl)(pentyl)amino)butyl)acetamide

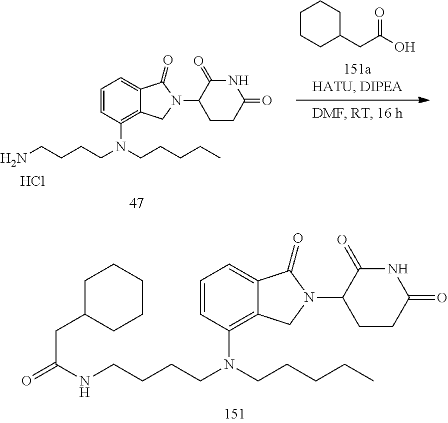 [SMILES: O=C(O)CC1CCCCC1 ; CCCCCN(CCCCN)c1cccc2c1CN(C1CCC(=O)NC1=O)C2=O ; Cl ; CCCCCN(CCCCNC(=O)CC1CCCCC1)c1cccc2c1CN(C1CCC(=O)NC1=O)C2=O]

To a stirred solution of 151a (0.150 g, 0.375 mmol, 1 equiv) in DMF (2 mL) were added DIPEA (0.262 ml, 1.498 mmol, 4 equiv) and HATU (0.214 g, 0.562 mmol, 1.5 equiv) at 0° C. 3-(4-((4-aminobutyl)(pentyl)amino)-1-oxoisoindolin-2-yl)piperidine-2,6-dione, HCl 47 (0.150 g, 0.375 mmol, 1 equiv) was then added at same temperature and the reaction mixture was stirred at room temperature for 16 h. Upon completion of the reaction (as monitored by LCMS), reaction mixture was purified by reverse-phase column chromatography (Grace® column: C18 40 μm, 40 g; flow rate: 20 mL/min; 0.1% aqueous HCOOH/ACN mobile phase). The pure fractions were lyophilized to afford 2-cyclohexyl-N-(4-((2-(2,6-dioxopiperidin-3-yl)-1-oxoisoindolin-4-yl)(pentyl)amino)butyl)acetamide 151 (95.22 mg, 0.180 mmol, 48.2% yield) as off white solid.

LCMS: 525.3 (M+H). Method: Column: Atlantis dC18 (50×4.6) 5μ, Mobile phase: A: 0.1% TFA in H₂O B: 0.1% TFA in ACN, Flow Rate: 1.5 ml/min. Rt (min): 1.912; Area %—99.21.

HPLC: 99.40%, Rt (min): 4.020. Method: Column: X-Bridge C8(50×4.6) mm, 3.5 μm, Mobile phase: A: 0.1% TFA in water, Mobile phase: B: ACN, Flow: 2.0 mL/min.

¹H-NMR (400 MHz, DMSO-d₆): δ 10.99 (s, 1H), 7.72 (s, 1H), 7.37 (t, J=7.60 Hz, 1H), 7.18 (d, J=7.20 Hz, 1H), 7.06 (d, J=8.40 Hz, 1H), 5.11 (q, J=4.80 Hz, 1H), 4.39 (d, J=16.80 Hz, 1H), 4.28 (d, J=16.80 Hz, 1H), 3.17 (d, J=6.00 Hz, 4H), 3.01 (d, J=6.00 Hz, 2H), 2.97-2.83 (m, 1H), 2.01 (t, J=6.00 Hz, 1H), 1.89 (d, J=6.80 Hz, 2H), 1.59 (d, J=8.80 Hz, 6H), 1.42 (d, J=6.80 Hz, 6H), 1.25 (s, 4H), 1.15 (t, J=12.40 Hz, 3H), 0.84 (t, J=7.20 Hz, 5H).

### Hydrochloride Salt Form of Compound 163

### 3-(4-((4-(benzylamino)butyl)(pentyl)amino)-1-oxoisoindolin-2-yl)piperidine-2,6-dione.HCl

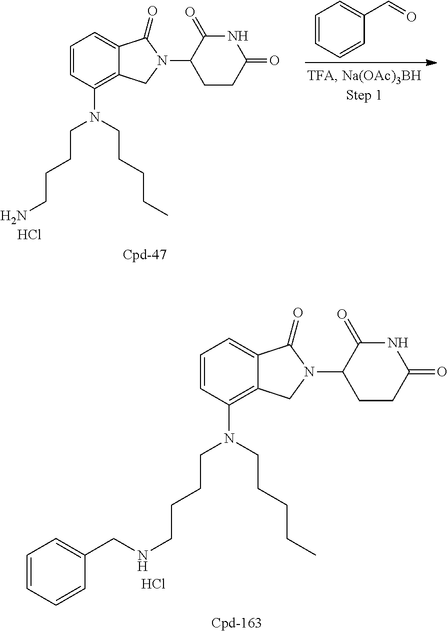 [SMILES: CCCCCN(CCCCN)c1cccc2c1CN(C1CCC(=O)NC1=O)C2=O ; O=Cc1ccccc1 ; Cl ; CCCCCN(CCCCNCc1ccccc1)c1cccc2c1CN(C1CCC(=O)NC1=O)C2=O ; Cl]

To a solution of 3-(4-((4-aminobutyl)(pentyl)amino)-1-oxoisoindolin-2-yl)piperidine-2,6-dione.HCl 47 (200 mg, 0.500 mmol, 1 equiv) and benzaldehyde (55.6 mg, 21.97 mmol, 1.05 equiv) in DCM (4 mL) and DMF (1 mL) was added TFA (0.034 ml, 0.430 mmol, 0.9 equiv) and Sodium triacetoxyborohydide (154 mg, 0.750 mmol, 1.5 equiv) was added in portions at 0° C. and the reaction mixture was stirred for 16 h at room temperature. Upon completion of the reaction (as confirmed by UPLC), the reaction mixture was diluted with DCM (20 mL), washed with water (2×10 mL), brine (10 mL), dried over anhy. Na₂SO₄, filtered and concentrated to give the crude product, which was purified by preparatory HPLC Column: XBridge C8—150; Method: HCl and ACN, flow rate: 20 ml/min) to give 3-(4-((4-(benzylamino)butyl)(pentyl)amino)-1-oxoisoindolin-2-yl)piperidine-2,6-dione 163 (29 mg, 0.058 mmol, 23.23% yield) as off white solid.

¹H-NMR (400 MHz, DMSO-d6): δ 10.99 (s, 1H), 9.08 (s, 2H), 7.52 (q, J=3.60 Hz, 2H), 7.41-7.45 (m, 4H), 7.26 (s, 2H), 5.12 (q, J=5.20 Hz, 1H), 4.60-4.20 (m, 2H), 4.09 (t, J=6.00 Hz, 2H), 3.22 (s, 2H), 2.92-2.87 (m, 3H), 2.63 (t, J=18.00 Hz, 1H), 2.33 (d, J=1.60 Hz, 1H), 1.64 (t, J=7.60 Hz, 2H), 1.45 (d, J=24.80 Hz, 4H), 1.28-1.21 (m, 4.8H), 0.84 (t, J=6.80 Hz, 3H).

LCMS: 491.2 (M+H), Method: Mobile phase:A:0.1% TFA in H₂O; Mobile phase:B: 0.1% TFA in ACN; Column:XBridge C8 (50×4.6 mm) 3.5 μm; Flow Rate:1.5 ml/min; Rt (min): 1.852, Area %: 98.144.

HPLC: Method: Mobile phase A:0.1% TFA in water, Mobile phase D:CAN; Flow rate:2.0 ml/min; Rt (min): 3.291, Area %: 95.870.

### Hydrochloride Salt Form of Compound 164

### 3-(1-oxo-4-(pentyl(4-(phenethylamino)butyl)amino)isoindolin-2-yl)piperidine-2,6-dione, HCl

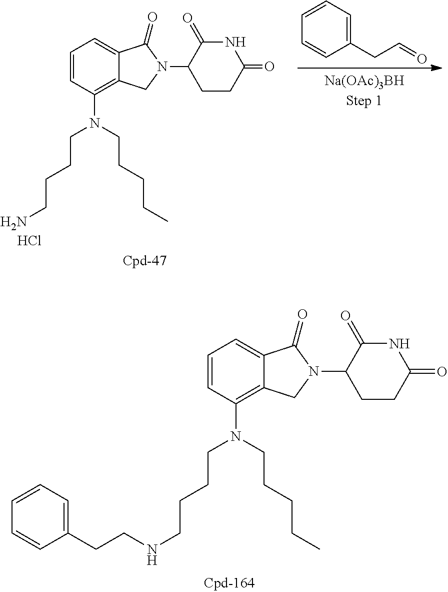

To a solution of 3-(4-((4-aminobutyl)(pentyl)amino)-1-oxoisoindolin-2-yl)piperidine-2,6-dione.HCl 47 (150 mg, 0.375 mmol, 1 equiv) in DCM (1.5 mL) and DMF (1.5 mL) were added molecular sieves (100 mg, 4 A° beads), 2-phenylacetaldehyde (45.0 mg, 0.375 mmol, 1 equiv) and sodium triacetoxyhydroborate (119 mg, 0.562 mmol, 1.5 equiv) at 0° C. The reaction mixture was warmed to room temperature and stirred for 16 h. Upon completion of the reaction (as confirmed by LCMS), the reaction mixture was filtered and the filtrate was lyophilised to give the crude product. The crude product was purified by preparatory HPLC (Column: XBridge C8-150; Method: HCl and ACN, flow rate: 20 ml/min) to give 3-(1-oxo-4-(pentyl(4-(phenethylamino)butyl)amino)isoindolin-2-yl)piperidine-2,6-dione, HCl (9 mg, 0.016 mmol, 4.37% yield)) as white solid.

¹H-NMR (400 MHz, DMSO-d6): δ 8.48 (s, 1H), 7.42-7.33 (m, 3H), 7.29-7.22 (m, 4H), 7.12 (d, J=8.40 Hz, 1H), 5.12 (q, J=4.80 Hz, 1H), 4.40 (d, J=16.80 Hz, 1H), 4.29 (d, J=17.20 Hz, 1H), 3.20 (s, 3H), 3.18 (s, 2H), 2.92 (q, J=8.80 Hz, 5H), 2.03-2.00 (m, 1H), 1.57 (t, J=7.20 Hz, 2H), 1.51-1.42 (m, 4H), 1.30-1.22 (m, 5H), 0.84 (t, J=6.80 Hz, 6H).

LCMS: 505.3 (M+H), Method: Mobile phase: A: 0.1% Formic Acid in H₂O B: ACN; Column: Atlantis dC18 (50×4.6) 5; Flow Rate:1.5 ml/min; Rt (min): 1.867, Area %: 98.821.

HPLC: Method: Mobile phase A:0.1% FA in water, Mobile phase D: ACN; Column: X Bridge C8(50×4.6) mm, 3.5 m, Flow rate:2.0 ml/min; Rt (min): 3.606, Area %: 98.493.

### Hydrochloride Salt Form of Compound 165

### 3-(1-oxo-4-(pentyl(4-((3-phenylpropyl)amino)butyl)amino)isoindolin-2-yl)piperidine-2,6-dione, HCl

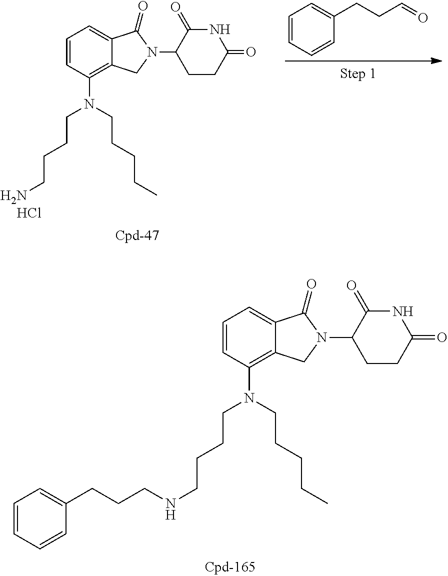

To a solution of 3-(4-((4-aminobutyl)(pentyl)amino)-1-oxoisoindolin-2-yl)piperidine-2,6-dione.HCl 47 (150 mg, 0.375 mmol, 1 equiv) in DCM (1.5 mL) and DMF (1 mL) were added molecular sieves (100 mg, 4 A° beads), 3-phenylpropanal (50.3 mg, 0.375 mmol, 1 equiv) and sodium triacetoxyhydroborate (119 mg, 0.562 mmol, 1.5 equiv) at 0° C. The reaction mixture was stirred for 16 h at room temperature. Upon completion of the reaction (as confirmed by LCMS), the reaction mixture was filtered and the filtrate was lyophilised to give the crude product. The crude product was purified by preparatory HPLC (Column: XBridge C8—150; Method: HCl and ACN, flow rate: 20 ml/min) to give 3-(1-oxo-4-(pentyl(4-((3-phenylpropyl)amino)butyl)amino)isoindolin-2-yl)piperidine-2,6-dione, HCl (18 mg, 0.031 mmol, 8.21% yield) as white solid.

¹H-NMR (400 MHz, DMSO-d6): δ 11.00 (s, 1H), 8.37 (s, 1H), 7.40 (t, J=7.60 Hz, 1H), 7.31 (t, J=7.60 Hz, 2H), 7.21 (q, J=2.80 Hz, 3H), 7.11 (d, J=8.40 Hz, 1H), 5.12 (q, J=5.20 Hz, 1H), 4.39 (d, J=16.80 Hz, 1H), 4.28 (d, J=16.40 Hz, 1H), 3.18 (q, J=6.40 Hz, 4H), 2.89 (q, J=7.60 Hz, 5H), 2.51 (q, J=1.60 Hz, 2H), 2.02 (t, J=5.20 Hz, 1H), 1.87 (t, J=7.60 Hz, 2H), 1.56-1.43 (m, 5H), 1.29-1.22 (m, 4H), 0.84 (t, J=6.80 Hz, 6H).

LCMS: 519.3 (M+H), Method: Mobile phase: A:0.1% Formic Acid in H2O B: ACN; Column: Atlantis dC18 (50×4.6) 5; Flow Rate:1.5 ml/min; Rt (min): 1.911, Area %: 96.329.

HPLC: Method: Mobile phase A:0.1% FA in water, Mobile phase D:ACN, Column: XBridge C8(50×4.6) mm, 3.5 m, Flow rate:2.0 ml/min, Rt (min): 3.785, Area %: 94.882.

### Hydrochloride Salt Form of Compound 166

### 3-(4-((4-(butylamino)butyl)(pentyl)amino)-1-oxoisoindolin-2-yl)piperidine-2,6-dione, HCl

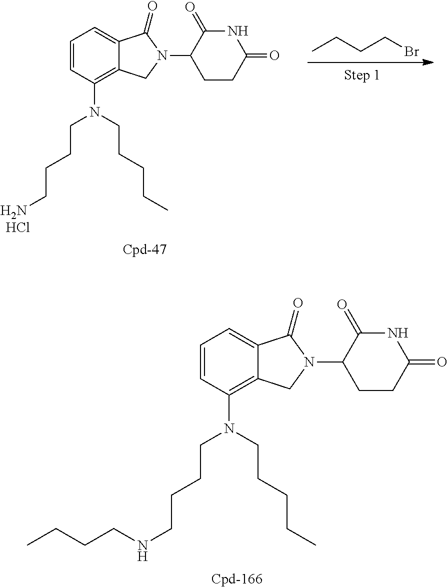

To a solution of 3-(4-((4-aminobutyl)(pentyl)amino)-1-oxoisoindolin-2-yl)piperidine-2,6-dione.HCl 47 (150 mg, 0.375 mmol, 1 equiv) in ACN (1 mL) was added TEA (41.7 mg, 0.412 mmol, 0.9 equiv) and 1-bromobutane (46.2 mg, 0.337 mmol, 1.1 equiv) was added in portions at 0° C. and the reaction mixture was stirred for 16 h at room temperature. Upon completion of the reaction (as confirmed by UPLC), the reaction mixture was diluted with DCM (20 mL), washed with water (2×10 mL), brine (10 mL), dried over anhy. Na₂SO₄, filtered and concentrated to give the crude product which was purified by preparatory HPLC (Column: YMC Triart C18; Method: HCl and ACN, flow rate: 20 ml/min) to give 3-(4-((4-(butylamino)butyl)(pentyl)amino)-1-oxoisoindolin-2-yl)piperidine-2,6-dione, HCl (28 mg, 0.056 mmol, 14.87% yield) as white solid.

¹H-NMR (400 MHz, DMSO-d6): δ 10.99 (s, 1H), 8.47 (s, 2H), 7.41 (t, J=7.60 Hz, 1H), 7.17 (t, J=12.00 Hz, 2H), 5.11 (t, J=5.20 Hz, 1H), 4.32-4.33 (m, 2H), 3.04 (m, 4H), 2.86-2.81 (m, 5H), 2.67 (d, J=1.60 Hz, 2H), 2.10 (s, 1H), 1.58-1.52 (m, 5H), 1.50-1.44 (m, 4H), 1.34-1.23 (m, 7H), 0.90-0.82 (m, 6H).

LCMS: 457.4 (M+H), Method: Mobile phase: A: 0.1% TFA in H₂O; Mobile phase: B: 0.1% TFA in ACN; Column: XBridge C8 (50×4.6 mm) 3.5 μm; Flow Rate: 1.5 ml/min; Rt (min): 2.345, Area %: 98.844.

HPLC: Method: Mobile phase A: 0.1% TFA in water, Mobile phase D: ACN; Column: XBridge C8(50×4.6) mm, 3.5 μm; Flow rate: 2.0 ml/min; Rt (min): 3.109, Area %: 98.065.

### Hydrochloride Salt Form of Compound 167

### 3-(4-((4-(isopentylamino)butyl)(pentyl)amino)-1-oxoisoindolin-2-yl)piperidine-2,6-dione. HCl

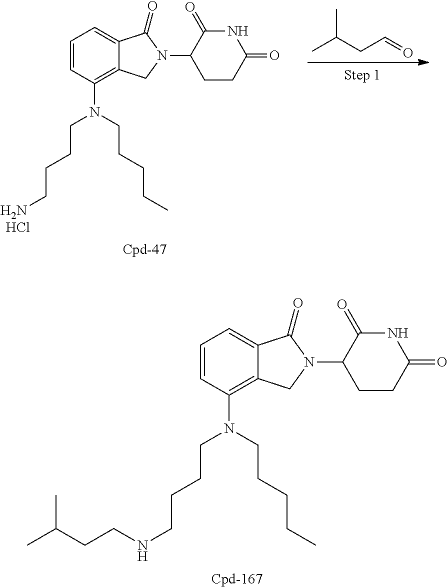

Step-1: 3-(4-((4-(isopentylamino)butyl)(pentyl)amino)-1-oxoisoindolin-2-yl)piperidine-2,6dione.HCl

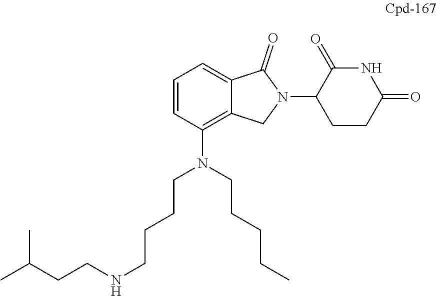 [SMILES: CCCCCN(CCCCNCCC(C)C)c1cccc2c1CN(C1CCC(=O)NC1=O)C2=O]

To a solution of 3-(4-((4-aminobutyl)(pentyl)amino)-1-oxoisoindolin-2-yl)piperidine-2,6-dione.HCl 47 (150 mg, 0.375 mmol, 1 equiv) in DCM (1.5 mL) and DMF (1.5 mL) was added molecular sieves (100 mg, 4 A° beads), 3-methylbutanal (32.3 mg, 0.375 mmol, 1 equiv) and sodium triacetoxyhydroborate (119 mg, 0.562 mmol, 1.5 equiv) at 0° C. The reaction mixture was stirred for 16 h at room temperature. Upon completion of the reaction (as confirmed by UPLC), the reaction mixture was diluted with DCM (20 mL), washed with water (2×10 mL), brine (10 mL), dried over anhy. Na₂SO₄, filtered and concentrated to give to the crude product. The crude product was purified by preparatory HPLC (Column: XBridge C8—150; Method: HCl and ACN, flow rate: 20 ml/min) to give 3-(4-((4-(isopentylamino)butyl)(pentyl)amino)-1-oxoisoindolin-2-yl)piperidine-2,6-dione (42 mg, 0.087 mmol, 23.34% yield) as white solid.

1HNMR (400 MHz, DMSO-d6): δ 11.02 (s, 1H), 8.89 (s, 2H), 7.48 (d, J=27.60 Hz, 2H), 5.13 (q, J=5.20 Hz, 1H), 4.49 (d, J=40.80 Hz, 2H), 2.82-2.52 (m, 4H), 2.07 (s, 1H), 1.52-1.58 (m, 9H), 1.24 (s, 5H), 0.85 (t, J=14.80 Hz, 9H).

LCMS: 471.4 (M+H), Method: Mobile phase: A: 0.1% Formic Acid in H₂O B: ACN; Column: Atlantis dC18 (50×4.6) 5μ; Flow Rate: 1.5 ml/min; Rt (min): 2.442, Area %: 98.217.

HPLC: Method: Mobile phase A: 0.1% TFA in water, Mobile phase D: ACN; Column: XBridge C8(50×4.6) mm, 3.5 μm, Flow rate: 2.0 ml/min; Rt (min): 3.357, Area %: 97.957.

### Compound 168

### Step-1: N-((1r,4r)-4-((2-(2,6-dioxopiperidin-3-yl)-1-oxoisoindolin-4-yl)(pentyl)amino)cyclohexyl)acetamide

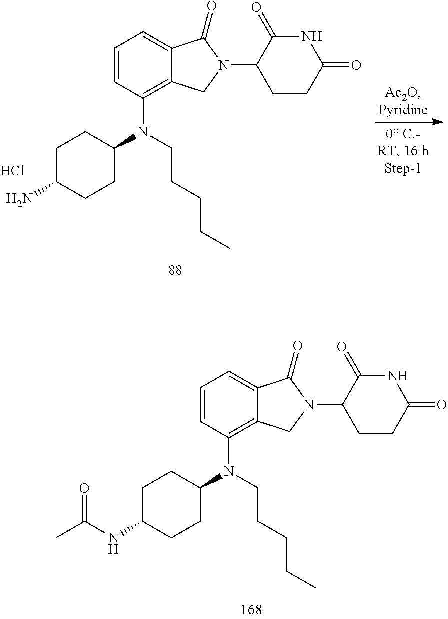 [SMILES: Cl ; CCCCCN(c1cccc2c1CN(C1CCC(=O)NC1=O)C2=O)[C@H]1CC[C@H](N)CC1 ; CCCCCN(c1cccc2c1CN(C1CCC(=O)NC1=O)C2=O)[C@H]1CC[C@H](NC(C)=O)CC1]

To a stirring solution of 3-(4-(((1r,4r)-4-aminocyclohexyl)(pentyl)amino)-1-oxoisoindolin-2-yl)piperidine-2,6-dione hydrochloride 88 (60 mg, 0.13 mmol) in pyridine (1 mL), was added Ac₂O (0.090 mL, 0.951 mmol) dropwise at 0° C. The reaction mixture was warmed to room temperature and stirred overnight. Upon completion of the reaction (as confirmed by UPLC), the reaction mixture was directly purified to preparatory HPLC purification (column: XSELECT C18-150, eluted with 0.1% aqueous formic acid and ACN with flow-rate of 15 mL/min.) to get the desired product N-((1r,4r)-4-((2-(2,6-dioxopiperidin-3-yl)-1-oxoisoindolin-4-yl)(pentyl)amino)cyclohexyl)acetamide 168 (12.45 mg, 0.027 mmol, 20.76% yield) as white solid.

¹H-NMR (400 MHz, DMSO-d6): δ 10.97 (s, 1H), 7.68 (d, J=7.60 Hz, 1H), 7.42 (t, J=7.60 Hz, 1H), 7.27 (q, J=7.60 Hz, 2H), 5.11 (dd, J=5.20, 13.20 Hz, 1H), 4.34 (d, J=17.20 Hz, 1H), 4.24 (d, J=17.60 Hz, 1H), 3.52-3.41 (m, 1H), 3.28-3.0 (m, 3H), 3.04 (m, 1H), 2.67-2.58 (m, 2H), 2.05-1.95 (m, 1H), 1.85-1.69 (m, 7H), 1.61-1.49 (m, 2H), 1.29-1.1 (m, 8H), 0.80 (t, J=6.80 Hz, 3H).

LCMS: 469.3 (M+H). Method: Column: XBridge C8 (50×4.6 mm) 3.5 μm, Mobile phase A: 0.1% TFA in H₂O, B: 0.1% TFA in ACN, Flow Rate: 1.5 mL/min, Rt (min): 1.431, Area (%): 99.61.

HPLC: Method info: A: 0.1% TFA in H₂O, B: ACN, Column: X-Bridge C8 (50 x4.6) mm, 3.5 μm; Flow Rate: 2.0 mL/min; Rt (min): 2.699; Area (%): 99.91.

### Hydrochloride Salt Form of Compound 169

### 3-(4-(((1R,4R)-4-(methylamino)cyclohexyl)(pentyl)amino)-1-oxoisoindolin-2-yl)piperidine-2,6-dione hydrochloride

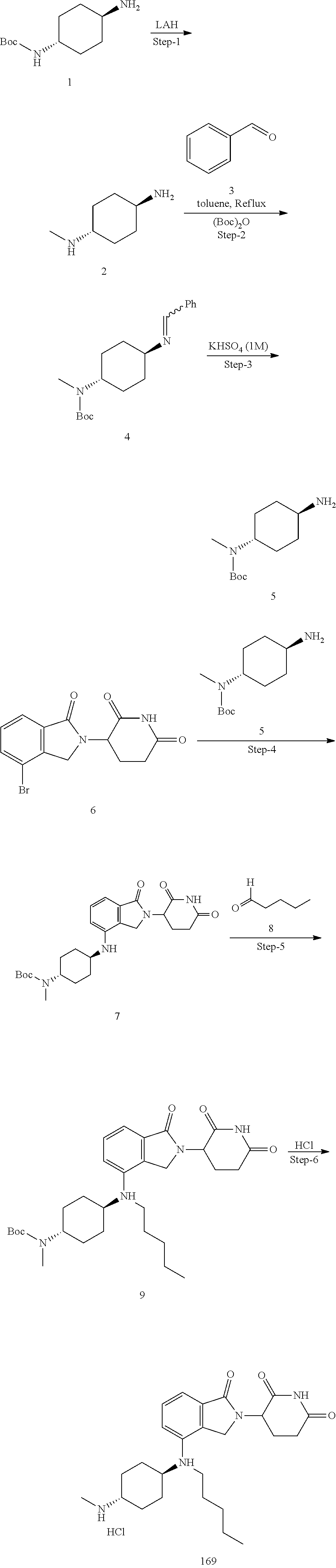

### Step-1: (1R,4R)-N1-methylcyclohexane-1,4-diamine

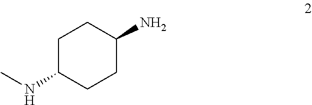

To a stirred solution of tert-butyl ((1R,4R)-4-aminocyclohexyl)carbamate 1 (7.45 g, 34.8 mmol, 1 equiv) in THF (120 mL) was added LAH (2M soln. in THF, 69.5 mL, 69.5 mmol, 2 equiv) drop-wise at 0° C. and stirred the reaction at room temperature for 30 min. Then the reaction mixture was refluxed for 2 h, cooled down to room temperature and stirred for overnight. Upon completion of the reaction (as confirmed by LCMS-ELSD) the reaction mixture was cooled to −5 to −10° C. and quenched slowly drop-wise with saturated solution of aqueous Na₂SO₄ (100 mL). The reaction mixture was stirred for 1 h at the same temperature. The resulted solid was filtered and washed with MTBE (2×200 mL) followed by DCM (2×200 mL). The filtrate was dried over anhydrous Na₂SO₄, filtered and concentrated under reduced pressure to afford the crude product 2 (1R,4R)-N1-methylcyclohexane-1,4-diamine 2 (4 g, 30.9 mmol, 89% yield) as off-white solid.

¹H-NMR (400 MHz, DMSO-d₆): δ 2.50-2.43 (m, 1H), 2.24 (s, 3H), 2.15-2.10 (m, 1H), 1.81-1.78 (m, 2H), 1.72-1.70 (m, 2H), 1.01-0.89 (m, 4H).

LCMS-ELSD: 129.2 (M+H), Rt (min): 0.506, Area (%): 99.656%.

### Step-2: tert-butyl ((1R,4R)-4-(((E)-benzylidene)amino)cyclohexyl)(methyl)carbamate

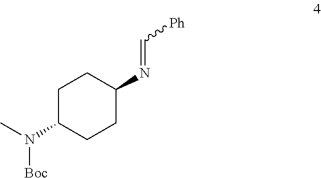 [SMILES: CN(C(=O)OC(C)(C)C)[C@H]1CC[C@H](N=Cc2ccccc2)CC1]

To a stirred solution of (1R,4R)-N1-methylcyclohexane-1,4-diamine 2 (4.0 g, 31.2 mmol, 1 equiv) in toluene (100 mL) was added benzaldehyde 3 (3.44 g, 32.4 mmol, 1 equiv) and heated to reflux using a Dean-Stark apparatus for 6 h. The reaction mixture was cooled to room temperature, Boc-anhydride (6.81 g, 31.2 mmol, 1 equiv) was added to the reaction mixture dropwise and continued stirring at room temperature for overnight. Upon completion of the reaction (monitored by 1HNMR and LCMS), the reaction mixture concentrated under reduced pressure to get the crude product tert-butyl ((1R,4R)-4-(((E)-benzylidene)amino)cyclohexyl)(methyl)carbamate 4 (11 g, 17.73 mmol, 56.8% yield) as colorless viscous liquid. The crude product was used for the next step without further purification.

LCMS: 317.3 (M+H), Rt (min):2.263, Area (%): 52.140.

### Step-3: tert-butyl ((1R,4R)-4-aminocyclohexyl)(methyl)carbamate

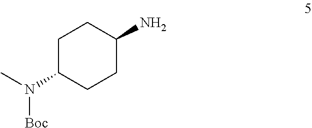 [SMILES: CN(C(=O)OC(C)(C)C)[C@H]1CC[C@H](N)CC1]

The crude product from previous step, tert-butyl ((1R,4R)-4-(((E)-benzylidene)amino)cyclohexyl)(methyl)carbamate 4 (11 g, 34.8 mmol, 1 equiv) was treated with 1M aqueous solution of KHSO₄ (20 mL, 20.00 mmol, 0.57 equiv) and stirred at room temperature for 4 h. After that the reaction mixture was extracted with MTBE (2×60 mL). The aqueous layer was basified with 1.5M aqueous solution of NaOH (10 mL), then extracted with DCM (3×250 mL). The combine organic layer was dried over anhydrous Na₂SO₄, filtered and concentrated under reduced pressure to afford crude product tert-butyl ((1R,4R)-4-aminocyclohexyl)(methyl)carbamate 5 (4 g, 17.47 mmol, 50.2% yield) as viscous colorless liquid.

¹H-NMR (400 MHz, DMSO-d6): δ 3.94 (br s, 1H), 2.76-2.65 (m, 3H), 2.64-2.60 (m, 1H), 1.94-1.91 (m, 2H), 1.71-1.70 (m, 2H), 1.68-1.61 (m, 2H), 1.52-1.52 (m, 11H), 1.48-1.32 (m, 2H).

LCMS: 229.3 (M+H), Rt (min):2.02, Area (%): 99.701.

### Step-4: tert-butyl ((1r,4r)-4-((2-(2,6-dioxopiperidin-3-yl)-1-oxoisoindolin-4-yl)amino)cyclohexyl)(methyl)carbamate

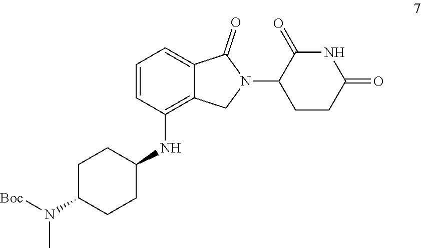 [SMILES: CN(C(=O)OC(C)(C)C)[C@H]1CC[C@H](Nc2cccc3c2CN(C2CCC(=O)NC2=O)C3=O)CC1]

To a solution of 3-(4-bromo-1-oxoisoindolin-2-yl)piperidine-2,6-dione 6 (1 g, 3.064 mmol, 1 equiv) in DMF (3 mL), were added tert-butyl ((1R,4R)-4-aminocyclohexyl)(methyl)carbamate 5 (1.554 g, 6.808 mmol, 2.2 equiv) and sodium tert-butoxide (0.582 g, 8.045 mmol, 2.6 equiv) at room temperature and de-gassed for 20 min by bubbling of nitrogen gas. Pd-PEPPSI-IHept(Cl) (0.18 g, 0.185 mmol, 0.06 equiv) was added to the reaction mixture under nitrogen atmosphere, de-gassed for 5 min and then heated at 120° C. under micro-wave for 1 h. Upon completion (as confirmed by LCMS), the reaction mixture was filtered through Celite-bed, washed with ethyl acetate (60 mL), concentrated to minimum volume (5 mL) and purified by reverse-phase column chromatography (Grace® column: C18 40 μm, 120 g; flow rate: 20 mL/min; 0.1% aqueous HCO₂H/CH₃CN mobile phase) to obtain the desired product tert-butyl ((1R,4R)-4-((2-(2,6-dioxopiperidin-3-yl)-1-oxoisoindolin-4-yl)amino)cyclohexyl)(methyl)carbamate 7 (0.228 g, 0.479 mmol, 31.0% yield) as yellow solid.

¹H-NMR (400 MHz, DMSO-d6): δ 11.02 (s, 1H), 7.28 (t, J=7.60 Hz, 1H), 6.92 (d, J=7.20 Hz, 1H), 6.81 (d, J=8.00 Hz, 1H), 5.31 (d, J=7.60 Hz, 1H), 5.12 (dd, J=5.20, 13.00 Hz, 1H), 4.22 (d, J=17.20 Hz, 1H), 4.12 (d, J=17.20 Hz, 1H), 3.84 (br s, 1H), 2.98-2.89 (m, 1H), 2.69 (s, 3H), 2.65-2.51 (m, 2H), 2.34-2.27 (m, 1H), 2.05-2.04 (m, 3H), 1.67-1.62 (m, 4H), 1.41 (s, 9H), 1.33-1.30 (m, 2H).

LCMS: 415.2 (M+H-56); Rt (min): 2.724; Area (%): 98.866.

### Step-5: tert-butyl ((1R,4R)-4-((2-(2,6-dioxopiperidin-3-yl)-1-oxoisoindolin-4-yl)(pentyl)amino)cyclohexyl)(methyl)carbamate

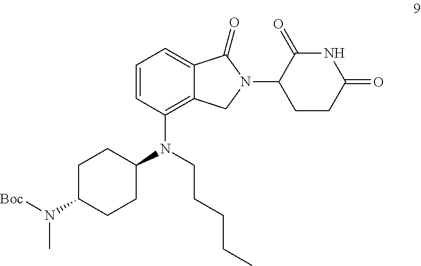 [SMILES: CCCCCN(c1cccc2c1CN(C1CCC(=O)NC1=O)C2=O)[C@H]1CC[C@H](N(C)C(=O)OC(C)(C)C)CC1]

Tert-butyl((1R,4R)4((2(2,6dioxopiperidin3yl)-1-oxoisoindolinyl)amino)cyclohexyl) (methyl) carbamate 7 (0.228 g, 0.485 mmol, 1 equiv) and pentanal 8 (0.209 g, 4.84 mmol, 5 equiv) were taken together in a mixture of DCM (3 mL) and DMF (3 mL) under nitrogen atmosphere. To this reaction mixture, TFA (0.166 g, 1.454 mmol, 5 equiv) was added and the reaction was stirred for 2 hour at room temperature. After that Sodium triacetoxyborohydride (0.308 g, 1.454 mmol, 5 equiv) was added in portions and the reaction mixture was stirred for additional 16 h at room temperature. The progress of reaction was monitored by TLC analysis (70% EtOAc Rf˜0.3, and LCMS), and found the SM was not consumed completely. So the reaction mixture was again cooled down to 0° C. and another lot of pentanal 8 (0.209 g, 2.423 mmol, 5 equiv) and TFA (0.075 ml, 0.969 mmol, 2 equiv) were added followed by addition of sodium triacetoxyborohydride (0.308 g, 1.454 mmol, 3 equiv) after 30 minutes and stirred at room temperature for 4 h. Upon completion (as confirmed by TLC analysis, 70% EtOAc in Pet-ether, R_{f}˜0.3) the reaction mixture was diluted with ice-cold water (150 mL), extracted with DCM (2×80 mL), the combined organic layer was washed with brine (50 mL), dried over anhydrous Na₂SO₄, filtered and concentrated to afford the crude product as white solid, which was purified by reverse-phase column chromatography (Grace® column: C18 40 μm, 120 g; flow rate: 20 mL/min; 0.1% aqueous HCO₂H/CH₃CN as mobile phase). The pure fractions were collected and lyophilized to obtain tert-butyl ((1R,4R)-4-((2-(2,6-dioxopiperidin-3-yl)-1-oxoisoindolin-4-yl)(pentyl)amino)cyclohexyl)(methyl)carbamate 9 (0.175 g, 0.322 mmol, 66.4% yield) as pale yellow solid.

¹H-NMR (400 MHz, DMSO-d6): δ 10.97 (s, 1H), 7.44-7.40 (m, 1H), 7.31-7.26 (m, 2H), 5.09 (dd, J=5.20, 13.20 Hz, 1H), 4.36 (d, J=17.20 Hz, 1H), 4.26 (d, J=16.80 Hz, 1H), 3.75-3.78 (m, 1H), 3.12-3.06 (m, 3H), 2.95-2.86 (m, 1H), 2.63 (s, 3H), 2.60-2.50 (m, 2H), 2.04-2.00 (m, 1H), 1.79-1.77 (m, 2H), 1.66-1.56 (m, 6H), 1.39 (s, 9H), 1.23-1.21 (m, 6H), 0.80 (t, J=6.80 Hz, 3H).

LCMS: 541.3 (M+H); Rt (min): 2.818; Area (%): 99.398.

### Step-6: 3-(4-(((1r,4r)-4-(methylamino)cyclohexyl)(pentyl)amino)-1-oxoisoindolin-2-yl)piperidine-2,6-dione hydrochloride

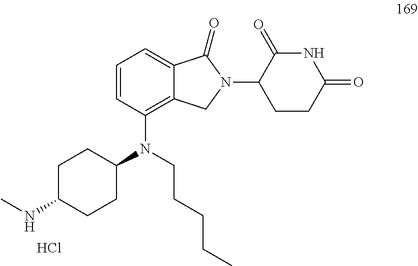 [SMILES: CCCCCN(c1cccc2c1CN(C1CCC(=O)NC1=O)C2=O)[C@H]1CC[C@H](NC)CC1 ; Cl]

To an ice cold solution of tert-butyl ((1r,4r)-4-((2-(2,6-dioxopiperidin-3-yl)-1-oxoisoindolin-4-yl)(pentyl)amino)cyclohexyl)(methyl)carbamate 9 (0.2 g, 0.370 mmol, 1 equiv) in DCM (2 mL) was added HCl (4M solution. in dioxane, 4 mL) and stirred for 1.5 h at room temperature. Upon completion (monitored by TLC analysis, 100% EtOAc Rf˜0.1) the reaction mixture was concentrated under reduced pressure, washed with MTBE (2×5 mL), dried under vacuum, dissolved in 1:1 mixture of ACN:H₂O (10 mL) and lyophilized to afford the desired product 3-(4-(((1r,4r)-4-(methylamino)cyclohexyl)(pentyl)amino)-1-oxoisoindolin-2-yl)piperidine-2,6-dione, hydrochloride 169 (0.145 g, 0.301 mmol, 81% yield) as yellow solid.

¹H-NMR (400 MHz, DMSO-d6): δ 10.98 (s, 1H), 8.68 (br s, 2H), 7.44-7.31 (m, 3H), 5.12 (dd, J=4.80 Hz, 1H), 4.33-4.27 (m, 2H), 3.10 (br s, 3H), 2.92-2.88 (m, 3H), 2.68-2.57 (m, 2H), 2.08-2.05 (m, 3H), 1.83 (br s, 2H), 1.53 (br s, 2H), 1.37-1.34 (m, 2H), 1.30-1.15 (m, 6H), 0.79 (t, J=6.40 Hz, 3H).

LCMS: 441.3 (M+H). Method: Column: Atlantis dC18 (50×4.6 mm) 5 μm; Mobile phase A: 0.1% FA in H₂O, Mobile phase B: ACN, Flow Rate: 1.5 mL/min. Rt (min): 1.653; Area (%): 98.685.

HPLC: Method: Column: X Bridge C8 (50×4.6) mm, 3.5 μm, Mobile Phase A: 0.1% TFA in H₂O, Mobile Phase B: ACN, Flow rate:2.0 mL/min. Rt (min): 2.372; Area (%): 99.770.

### Compound 181

The compound having the following structure is prepared in analogy from the preparation procedure described for the above-mentioned compounds.

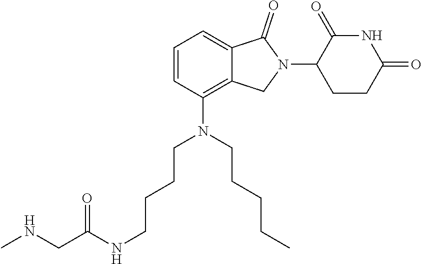 [SMILES: CCCCCN(CCCCNC(=O)CNC)c1cccc2c1CN(C1CCC(=O)NC1=O)C2=O]

### Compound 223

The compound having the following structure is prepared in analogy from the preparation procedure described for the above-mentioned compounds.

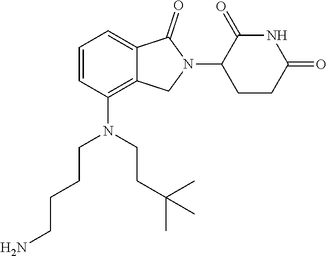 [SMILES: CC(C)(C)CCN(CCCCN)c1cccc2c1CN(C1CCC(=O)NC1=O)C2=O]

### Hydrochloride Salt Form of Compound 235

### 3-(4-(((1r,4r)-4-aminocyclohexyl)(cyclopropylmethyl)amino)-1-oxoisoindolin-2-yl)piperidine-2,6-dione hydrochloride

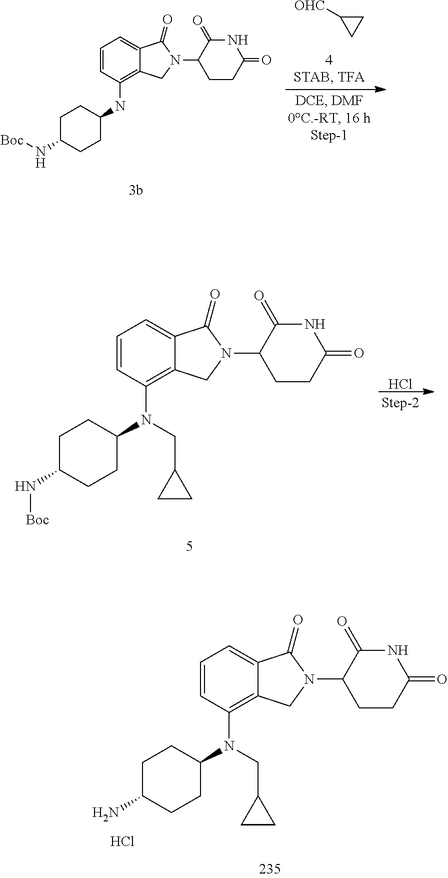 [SMILES: CC(C)(C)OC(=O)N[C@H]1CC[C@H](Nc2cccc3c2CN(C2CCC(=O)NC2=O)C3=O)CC1 ; O=CC1CC1 ; CC(C)(C)OC(=O)N[C@H]1CC[C@H](N(CC2CC2)c2cccc3c2CN(C2CCC(=O)NC2=O)C3=O)CC1 ; Cl ; N[C@H]1CC[C@H](N(CC2CC2)c2cccc3c2CN(C2CCC(=O)NC2=O)C3=O)CC1 ; Cl]

### Step-1: tert-butyl ((1r,4r)-4-((cyclopropylmethyl)(2-(2,6-dioxopiperidin-3-yl)-1-oxoisoindolin-4-yl)amino)cyclohexyl)carbamate

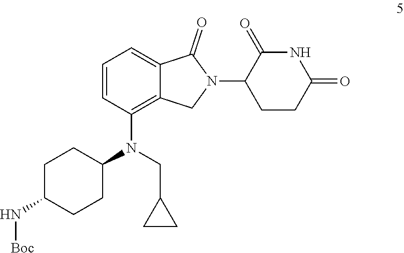 [SMILES: CC(C)(C)OC(=O)N[C@H]1CC[C@H](N(CC2CC2)c2cccc3c2CN(C2CCC(=O)NC2=O)C3=O)CC1]

To a stirred solution of tert-butyl ((1r,4r)-4-((2-(2,6-dioxopiperidin-3-yl)-1-oxoisoindolin-4-yl)amino)cyclohexyl)carbamate 3b (0.1 g, 0.219 mmol, 1 equiv) and cyclopropanecarbaldehyde 4 (0.184 g, 2.628 mmol, 12 equiv) in DCE (2 mL), DMF (0.2 mL) were added TFA (0.05 mL, 0.658 mmol, 3 equiv) and sodium triacetoxyborohydride (0.278 g, 1.314 mmol, 6 equiv) at 0° C. The resulting reaction mixture was stirred at room temperature overnight. Upon completion of the reaction (as confirmed by LCMS analysis) the reaction mixture was poured into ice cold water (10 mL) and extracted with DCM (3×15 mL). The combined organic layer was washed with brine (10 mL), dried over anhydrous sodium sulphate, filtered and concentrated to get the crude product. The crude compound was purified by preparative HPLC (Column: XSelect C18-250, Mobile phase: 0.1% FA in Water/ACN, Flow-rate: 15 mL/min), pure fractions were collected and lyophilized to get the desired product tert-butyl ((1r,4r)-4-((cyclopropylmethyl)(2-(2,6-dioxopiperidin-3-yl)-1-oxoisoindolin-4-yl)amino)cyclohexyl)carbamate 5 (65 mg, 0.127 mmol, 57.99% yield) as off-white solid.

¹H-NMR (400 MHz, DMSO-d6): 10.94 (s, 1H), 7.42-7.29 (m, 3H), 6.65 (d, J=7.60 Hz, 1H), 5.07 (q, J=4.80 Hz, 1H), 4.32 (d, J=17.20 Hz, 1H), 4.23 (d, J=17.20 Hz, 1H), 3.27-3.11 (m, 1H), 2.97-2.83 (m, 4H), 2.57-2.47 (m, 2H), 1.99-1.96 (m, 1H), 1.80-1.65 (m, 4H), 1.38-1.32 (m, 11H), 1.20-1.12 (m, 2H), 0.65 (t, J=4.80 Hz, 1H), 0.25 (t, J=4.40 Hz, 2H), 0.04 (d, J=3.60 Hz, 2H).

LCMS: 511.3 (M+H). Method: Column: Atlantis dC18 (50×4.6) 5μ, Mobile phase A: 0.1% FA in H₂O, B: ACN, Flow Rate: 1.5 mL/min, Rt (min): 2.073, Area (%): 94.454.

HPLC: Method info A: 0.1% TFA in H₂O, B: ACN, Column: XBridge C8 (50 x4.6) mm, 3.5 μm; Flow Rate: 2.0 mL/min; Rt (min): 3.033; Area (%): 98.038.

### Step-2: 3-(4-(((1r,4r)-4-aminocyclohexyl)(cyclopropylmethyl)amino)-1-oxoisoindolin-2-yl)piperidine-2,6-dione hydrochloride

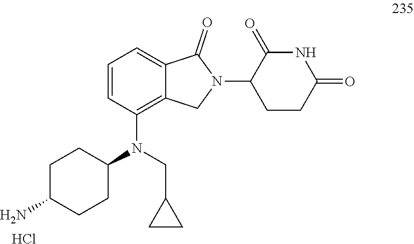 [SMILES: N[C@H]1CC[C@H](N(CC2CC2)c2cccc3c2CN(C2CCC(=O)NC2=O)C3=O)CC1 ; Cl]

To a stirred solution of tert-butyl ((1r,4r)-4-((cyclopropylmethyl)(2-(2,6-dioxopiperidin-3-yl)-1-oxoisoindolin-4-yl)amino)cyclohexyl)carbamate 5 (65 mg, 0.127 mmol, 1 equiv) in DCM (2 mL) was added 4N HCl in EtOAc (0.5 mL, 2 mmol, 15.7 equiv) at 0° C. The resultant reaction mixture was allowed to stir at room temperature for 5 h. Upon completion of the reaction (as confirmed by TLC analysis, 100% EtOAc, Rf˜0.1), the reaction mixture was concentrated under reduced pressure to get the crude product. The crude compound was washed by EtOAc (2 mL), dried under vacuum, dissolved in 3 mL of ACN-water mixture (1:1) and lyophilized to get the desired product 3-(4-(((1r,4r)-4-aminocyclohexyl)(cyclopropylmethyl)amino)-1-oxoisoindolin-2-yl)piperidine-2,6-dione hydrochloride 235 (47 mg, 0.102 mmol, 80.31% yield) as off-white solid.

¹H-NMR (400 MHz, DMSO-d₆): δ 10.99 (s, 1H), 7.94 (br s, 3H), 7.44-7.42 (m, 3H), 5.13 (q, J=4.00 Hz, 1H), 4.37 (d, J=15.60 Hz, 1H), 4.28 (d, J=17.20 Hz, 1H), 3.48-2.90 (m, 5H), 2.67-2.53 (m, 2H), 2.09-1.92 (m, 3H), 1.89-1.76 (m, 2H), 1.54-1.24 (m, 4H), 1.75-1.60 (m, 1H), 0.31-0.29 (m, 2H), 0.10-0.04 (m, 2H).

LCMS: 411.3 (M+H). Method: Column: Atlantis dC18 (50×4.6) 5μ, Mobile phase A: 0.1% FA in H₂O, B: ACN, Flow Rate: 1.5 mL/min, Rt (min): 1.845, Area (%): 97.283.

HPLC: Method info A: 0.1% TFA in H₂O, B: ACN, Column: XBridge C8 (50 x4.6) mm, 3.5 μm; Flow Rate: 2.0 mL/min; Rt (min): 1.698; Area (%): 97.169.

### Hydrochloride Salt Form of Compound 236

### 3-(4-(((1r,4r)-4-aminocyclohexyl)(isobutyl)amino)-1-oxoisoindolin-2-yl)piperidine-2,6-dione. HCl

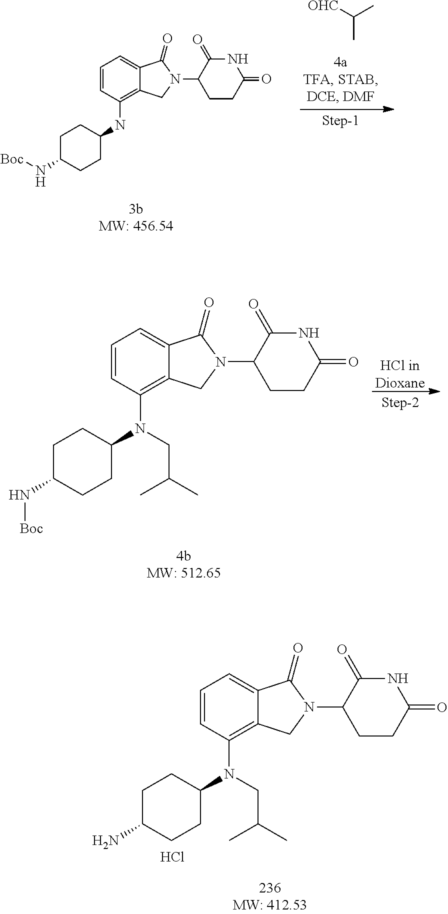

### Step-1: tert-butyl((1r,4r)-4-((2-(2,6-dioxopiperidin-3-yl)-1-oxoisoindolin-4-yl)(isobutyl)amino)cyclohexyl)carbamate

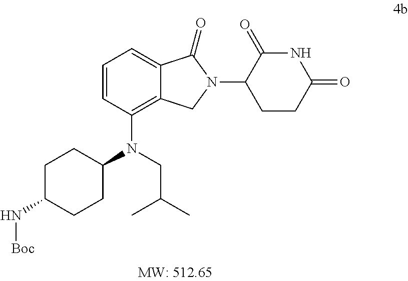 [SMILES: CC(C)CN(c1cccc2c1CN(C1CCC(=O)NC1=O)C2=O)[C@H]1CC[C@H](NC(=O)OC(C)(C)C)CC1]

A mixture of tert-butyl ((1r,4r)-4-((2-(2,6-dioxopiperidin-3-yl)-1-oxoisoindolin-4-yl)amino)cyclohexyl)carbamate 3b (100 mg, 0.219 mmol, 1 equiv) and isobutyraldehyde (237 mg, 3.29 mmol, 15 equiv) were dissolved in a mixture of 1,2-dichloroethane (1.5 mL) and DMF (1.5 mL) under nitrogen atmosphere. To this solution, TFA (37.5 mg, 0.329 mmol, 1.5 equiv) and Na(OAc)₃BH (232 mg, 1.095 mmol, 5 equiv) were added in portions at 0° C. and stirred for 16 h at room temperature. Upon completion of the reaction (as confirmed by LCMS), the reaction mixture was diluted with DCM (10 mL), washed with water (3×10 mL), brine (10 mL), dried over Na₂SO₄, filtered and concentrated to give the crude product which was purified by Preparative HPLC (Column: X-BRIDGE-C18, 0.1% HCOOH in water, flow rate: 20 mL/min). The pure fractions were lyophilised to give tert-butyl ((1r,4r)-4-((2-(2,6-dioxopiperidin-3-yl)-1-oxoisoindolin-4-yl)(isobutyl)amino)cyclohexyl)carbamate (38 mg, 0.071 mmol, 24.22% yield) as off white solid.

¹H-NMR (400 MHz, DMSO-d6): δ 10.96 (s, 1H), 7.41 (t, J=7.60 Hz, 1H), 7.30 (q, J=4.80 Hz, 2H), 6.66 (d, J=7.60 Hz, 1H), 4.43 (d, J=6.40 Hz, 1H), 4.28 (d, J=14.80 Hz, 2H), 3.84 (s, 2H), 3.12 (d, J=2.40 Hz, 2H), 2.89 (q, J=7.60 Hz, 4H), 2.61 (s, 2H), 1.80 (t, J=8.40 Hz, 6H), 1.47 (d, J=44.00 Hz, 3H), 1.14 (s, 13H), 1.10 (q, J=1.20 Hz, 10H), 0.91 (d, J=6.80 Hz, 4H), 0.80 (q, J=4.00 Hz, 17H).

LCMS: 513.3 (M+H), Rt (min): 2.588, Area (%): 98.566.

HPLC: Rt (min): 4.502, Area (%): 95.624.

### Step-2: 3-(4-(((1r,4r)-4-aminocyclohexyl)(isobutyl)amino)-1-oxoisoindolin-2-yl)piperidine-2,6-dione. HCl

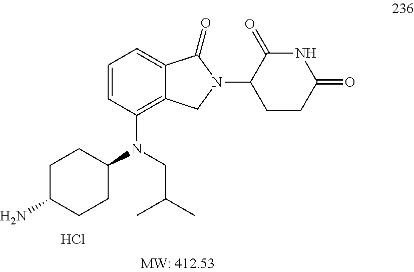 [SMILES: CC(C)CN(c1cccc2c1CN(C1CCC(=O)NC1=O)C2=O)[C@H]1CC[C@H](N)CC1 ; Cl]

To an ice cold solution of tert-butyl ((1r,4r)-4-((2-(2,6-dioxopiperidin-3-yl)-1-oxoisoindolin-4-yl)(isobutyl)amino)cyclohexyl)carbamate 4b (38 mg, 0.074 mmol) in DCM (1 ml) was added HCl (4M in Ethyl acetate, 0.093 ml) was added and was stirred for 3 h at room temperature. Upon completion of reaction (as confirmed by LCMS), the reaction mixture was concentrated and the solid was triturated with MTBE (10 ml), dried under vacuum and lyophilised to get 3-(4-(((1r,4r)-4-aminocyclohexyl)(isobutyl)amino)-1-oxoisoindolin-2-yl)piperidine-2,6-dione.HCl (8.26 mg, 0.020 mmol, 26.6% yield) as off white solid.

¹H-NMR (400 MHz, DMSO-d6): δ 10.98 (s, 1H), 7.83 (s, 3H), 7.43 (t, J=7.60 Hz, 1H), 7.32 (t, J=6.80 Hz, 2H), 5.13 (q, J=5.20 Hz, 1H), 4.29 (q, J=17.20 Hz, 2H), 2.97-2.87 (m, 5H), 2.51 (s, 1H), 1.98 (q, J=11.20 Hz, 4H), 1.77 (s, 2H), 1.61 (d, J=12.40 Hz, 3H), 1.46-1.32 (m, 4H), 0.87 (t, J=10.40 Hz, 6H).

LCMS: 413.2 (M+H), Method: Mobile phase: A: 0.1% FA in H₂O, Mobile phase: B: ACN, Column: Atlantis dC18 (50×4.6 mm) 5 μm, Flow Rate: 1.5 ml/min Rt (min): 1.653, Area %: 99.413.

HPLC: Method: Mobile phase: A: 0.1% TFA in water, Mobile phase: B: ACN, Column: X-Bridge C8(50×4.6) mm, 3.5 μm Flow: 2.0 mL/min, Rt (min): 2.653, Area %: 98.413.

### Hydrochloride Salt form of Compound 237

### 3-(4-(((1r,4r)-4-aminocyclohexyl)(2-cyclopropylethyl)amino)-1-oxoisoindolin-2-yl)piperidine-2,6-dione, HCl

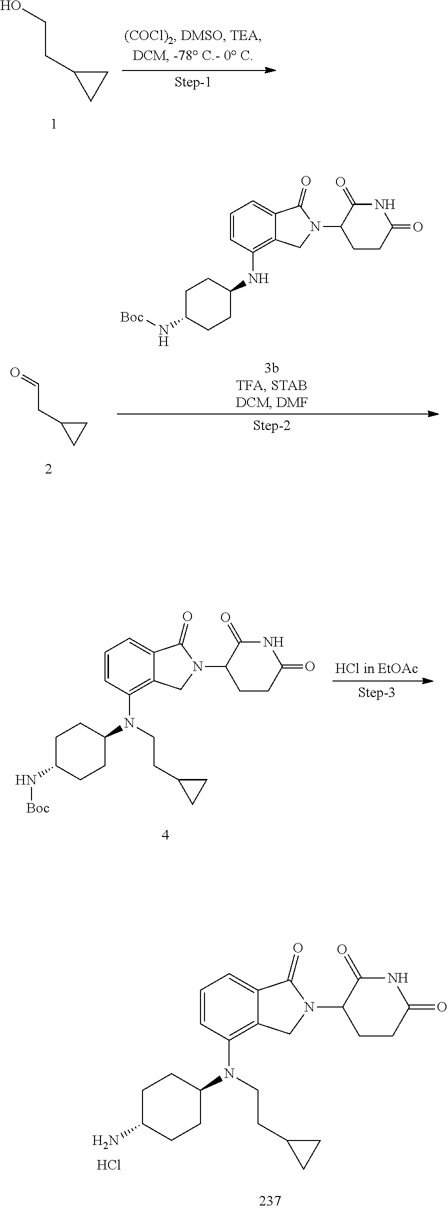 [SMILES: OCCC1CC1 ; CC(C)(C)OC(=O)N[C@H]1CC[C@H](Nc2cccc3c2CN(C2CCC(=O)NC2=O)C3=O)CC1 ; O=CCC1CC1 ; CC(C)(C)OC(=O)N[C@H]1CC[C@H](N(CCC2CC2)c2cccc3c2CN(C2CCC(=O)NC2=O)C3=O)CC1 ; Cl ; N[C@H]1CC[C@H](N(CCC2CC2)c2cccc3c2CN(C2CCC(=O)NC2=O)C3=O)CC1]

### Step-1: 2-cyclopropylacetaldehyde

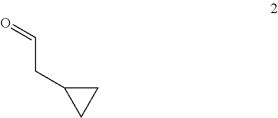

To a stirred solution of oxalyl chloride (1.016 mL, 11.61 mmol, 2.0 equiv) in DCM (10.0 mL) was added a solution of DMSO (1.648 mL, 23.22 mmol, 4.0 equiv) in DCM (2.00 mL) dropwise at −78° C. under nitrogen atmosphere. The solution was stirred for 30 min at −78° C. and a solution of 2-cyclopropylethan-1-ol 1 (0.500 g, 5.80 mmol, 1.0 equiv) in DCM (2.00 ml) was added dropwise. The resulting solution was stirred 1.5 h at −78° C., then added triethylamine (4.85 mL, 34.8 mmol, 6.0 equiv) as dropwise at −78° C. Then the reaction mixture was slowly warmed to 0° C. and stirred for 1 h. Upon completion of the reaction (as confirmed by TLC analysis, 10% EtOAc in pet ether, R_{f}˜0.3), the reaction mixture was quenched with ice cold 1N aqueous HCl solution (25 mL) at 0° C. and diluted with DCM (5 mL). The organic layer was separated and the aqueous layer was extracted with DCM (1×10 mL). The combined organic layer was washed with saturated aqueous NaHCO₃ solution (35 mL) followed by brine (50 mL), dried over anhydrous sodium sulphate, filtered and concentrated under reduced pressure at 0° C. to half of the volume to afford the crude of 2-cyclopropylacetaldehyde 2 (6.5 g) in DCM as pale yellow liquid.

¹H-NMR (400 MHz, DMSO-d₆): δ 9.82 (d, J=2.40 Hz, 1H), 2.13 (s, 2H), 1.00 (m, 1H), 0.65-0.60 (m, 2H), 0.21-0.17 (m, 2H).

### Step-2: tert-butyl ((1r,4r)-4-((2-cyclopropylethyl)(2-(2,6-dioxopiperidin-3-yl)-1-oxoisoindolin-4-yl)amino)cyclohexyl)carbamate

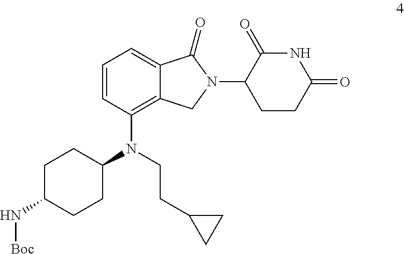 [SMILES: CC(C)(C)OC(=O)N[C@H]1CC[C@H](N(CCC2CC2)c2cccc3c2CN(C2CCC(=O)NC2=O)C3=O)CC1]

To a stirred solution of tert-butyl ((1r,4r)-4-((2-(2,6-dioxopiperidin-3-yl)-1-oxoisoindolin-4-yl)amino)cyclohexyl)carbamate 3b (0.12 g, 0.263 mmol, 1.0 equiv) and 2-cyclopropylacetaldehyde 2 (0.221 g, 2.63 mmol, 10.0 equiv) in a mixture of DCE (5 mL) and DMF (0.2 mL) were added trifluoroacetic acid (0.090 g, 0.789 mmol, 3.0 equiv) and sodium triacetoxyborohydride (0.279 g, 1.314 mmol, 5 equiv) at 0° C. The resulting reaction mixture was stirred at room temperature overnight. Upon completion of the reaction (as confirmed by LCMS analysis) the reaction mixture was poured into ice cold water (15 mL) and extracted with EtOAc (3×20 mL). The combined organic layer was washed with brine (50 mL), dried over anhydrous sodium sulphate, filtered and concentrated to get the crude product. The crude compound was purified by preparative HPLC (column: XSelect C18-19.1×150 mm, 500 μl, Mobile phase: 0.1% FA in water/ACN, Flow-rate: 15 mL/min), pure fractions were collected and lyophilized to afford the desired product tert-butyl ((1r,4r)-4-((2-cyclopropylethyl)(2-(2,6-dioxopiperidin-3-yl)-1-oxoisoindolin-4-yl)amino)cyclohexyl)carbamate 4 (0.038 g, 0.070 mmol, 26.5% yield) as off-white solid.

¹H-NMR (400 MHz, DMSO-d₆): δ 10.98 (s, 1H), 7.41 (t, J=7.6 Hz, 1H), 7.30-7.24 (m, 2H), 6.70 (d, J=7.6 Hz, 1H), 5.10 (q, J=5.20 Hz, 1H), 4.33 (d, J=17.2 Hz, 1H), 4.23 (d, J=17.2 Hz, 1H), 3.35 (m, 3H), 3.02 (m, 1H), 2.90 (m, 1H), 2.61 (s, 1H), 2.02-1.99 (m, 1H), 1.81-1.73 (m, 4H), 1.55 (d, J=12.00 Hz, 2H), 1.39 (s, 10H), 1.24-1.13 (m, 4H), 0.64 (m, 1H), 0.35-0.31 (m, 2H), −0.05-−0.08 (m, 2H).

LCMS: 525.3 (M+H), Method: Mobile phase A: 0.1% FA in water, Mobile phase B: ACN, Column: Atlantis dC18 (50×4.6 mm) 5 μm, Flow Rate: 1.5 ml/min Rt (min): 2.481, Area (%): 98.400.

HPLC: Method: Mobile phase A: 0.1% TFA in water, Mobile phase B: ACN, Column: Xbridge C8(50×4.6) mm, 3.5 m, Flow: 2.0 mL/min, Rt (min): 3.414, Area %: 96.143.

### Step-3:3-(4-(((1r,4r)-4-aminocyclohexyl)(phenethyl)amino)-1-oxoisoindolin-2-yl)piperidine-2,6-dione, HCl

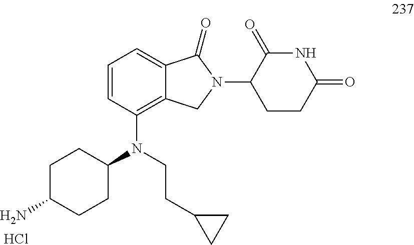 [SMILES: Cl ; N[C@H]1CC[C@H](N(CCC2CC2)c2cccc3c2CN(C2CCC(=O)NC2=O)C3=O)CC1]

To a stirred solution of tert-butyl ((1r,4r)-4-((2-cyclopropylethyl)(2-(2,6-dioxopiperidin-3-yl)-1-oxoisoindolin-4-yl)amino)cyclohexyl)carbamate 4 (0.035 g, 0.067 mmol, 1 equiv) in DCM (2 mL) was added 4N HCl in EtOAc (0.334 mL, 1.334 mmol, 20 equiv) at 0° C. The resultant reaction mixture was warmed to room temperature and stirred for 3 h. Upon completion of the reaction (as confirmed by LCMS analysis and TLC analysis, 100% EtOAc, Rf˜0.1), the reaction mixture was concentrated under reduced pressure to get the crude product as pale brown gummy solid. The obtained solid was washed with MTBE (10 mL), dried under vacuum and lyophilized to afford desired product 3-(4-(((1r,4r)-4-aminocyclohexyl)(2-cyclopropylethyl)amino)-1-oxoisoindolin-2-yl)piperidine-2,6-dione, HCl 237 (0.028 g, 0.058 mmol, 87% yield) as pale beige solid.

¹H-NMR (400 MHz, DMSO-d₆): δ 10.97 (s, 1H), 7.90 (s, 3H), 7.44-7.18 (m, 3H), 5.14-5.10 (m, 1H), 4.32-4.26 (m, 2H), 3.22 (s, 3H), 2.97-2.88 (m, 2H), 2.68-2.62 (m, 2H), 2.02-1.96 (m, 3H), 1.80 (s, 2H), 1.61 (d, J=12.00 Hz, 2H), 1.41-1.35 (m, 2H), 1.14 (s, 2H), 0.63 (d, J=6.40 Hz, 1H), 0.36-0.32 (m, 2H), −0.04-−0.08 (m, 2H).

LCMS: 425.2 (M+H), Method: Mobile phase A: 0.1% FA in water, Mobile phase B: ACN, Column: Atlantis dC18 (50×4.6 mm) 5 μm, Flow Rate: 1.5 ml/min Rt (min): 1.549, Area (%): 94.563.

HPLC: Method: Mobile phase A: 0.1% TFA in water, Mobile phase B: ACN, Column: X Bridge C8 (50×4.6) mm, 3.5 μm, Flow Rate: 2.0 mL/min, Rt (min): 2.233, Area (%): 95.685.

### Hydrochloride Salt Form of Compound 238

### 3-(4-(((1r,4r)-4-aminocyclohexyl)(isopentyl)amino)-1-oxoisoindolin-2-yl)piperidine-2,6-dione hydrochloride

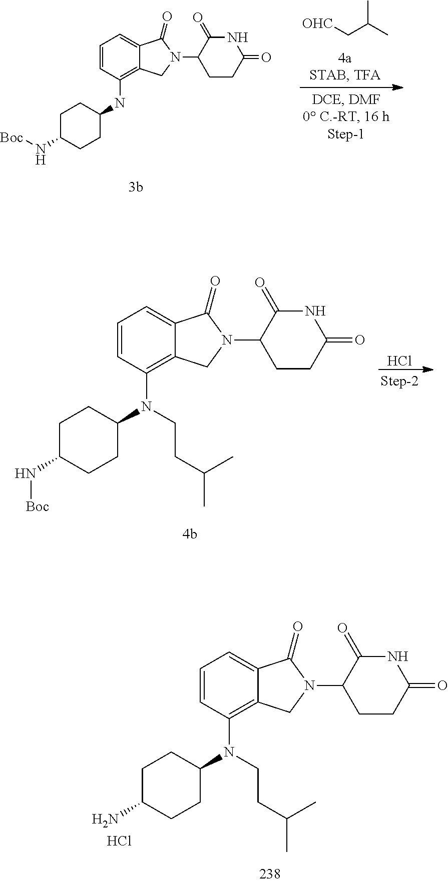

### Step-1: tert-butyl ((1r,4r)-4-((2-(2,6-dioxopiperidin-3-yl)-1-oxoisoindolin-4-yl)(isopentyl)amino)cyclohexyl)carbamate

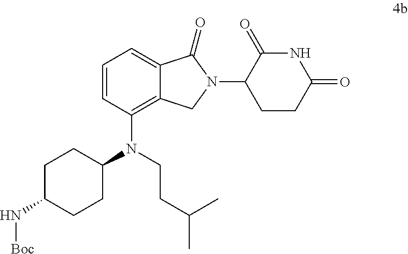 [SMILES: CC(C)CCN(c1cccc2c1CN(C1CCC(=O)NC1=O)C2=O)[C@H]1CC[C@H](NC(=O)OC(C)(C)C)CC1]

To a stirred solution of tert-butyl ((1r,4r)-4-((2-(2,6-dioxopiperidin-3-yl)-1-oxoisoindolin-4-yl)amino)cyclohexyl)carbamate 3b (0.1 g, 0.219 mmol, 1 equiv) and 3-methylbutanal 4a (0.132 g, 1.533 mmol, 6 equiv) in a mixture of DCE (1.5 mL) and DMF (1.5 mL) under nitrogen atmosphere at 0° C. was added 2,2,2-trifluoroacetic acid (0.025 mL, 0.329 mmol, 1.5 equiv) and stirred for 10 minutes. After that sodium triacetoxyhydroborate (0.139 g, 0.657 mmol, 3 equiv) was added and the reaction mixture was stirred overnight at room temperature. The progress of the reaction was monitored by TLC (100% ethyl acetate; Rf˜0.5) and LCMS which indicated presence of un-reacted starting material. The reaction mixture was again cooled to 0° C. and additional quantities of 3-methylbutanal 4a (0.132 g, 1.533 mmol, 7 equiv), TFA (0.025 ml, 0.329 mmol, 1.8 equiv) were added followed by sodium triacetoxyborohydride (0.139 g, 0.657 mmol, 3 equiv). The reaction mixture was then stirred overnight at room temperature. Upon completion of reaction (as confirmed by LCMS). The reaction mixture was quenched with ice-water (20 mL) and extracted with DCM (2×30 mL). The combined organic layer was washed with brine (30 mL), dried over anhydrous sodium sulphate, filtered and concentrated under reduced pressure to get the crude product. The crude product was purified by preparative-HPLC (Column: X-Bridge-C18-150, Additional column: XSelect C18-19.1×150 mm, Mobile phase:0.1% FA in Water/ACN, Flow-rate: 15 mL/min) pure fractions were collected and lyophilized to afford tert-butyl ((1r,4r)-4-((2-(2,6-dioxopiperidin-3-yl)-1-oxoisoindolin-4-yl) (isopentyl)amino) cyclohexyl) carbamate 4b (0.033 g, 0.064 mmol, 33.8% yield) as white solid.

¹H-NMR (400 MHz, DMSO-d₆): δ 10.97 (s, 1H), 7.43 (d, J=7.20 Hz, 1H), 7.33-7.29 (m, 2H), 6.69 (d, J=6.80 Hz, 1H), 5.11 (q, J=4.80 Hz, 1H), 4.33 (d, J=16.40 Hz, 1H), 4.24 (d, J=16.40 Hz, 1H), 3.25-3.1 (br s, 3H), 2.86-2.99 (m, 2H), 2.68-2.67 (m, 2H), 2.03-2.00 (m, 1H), 1.81-1.73 (m, 4H), 1.57-1.51 (m, 3H), 1.37 (s, 9H), 1.23-1.19 (m, 4H), 1.16-1.11 (m, 6H).

LCMS: 527.6 (M+H). Method: Mobile Phase A: 0.1% TFA in water. Mobile Phase B: 0.1% TFA ACN. Flow Rate: 1.5 mL/min. Column: XBridge C8 (50×4.6 mm) 3.5 μm, Rt (min): 1.881; Area (%): 99.298.

HPLC Method: Mobile Phase A: 0.1% TFA in water. Mobile Phase B: ACN. Flow rate: 2.0 mL/min. Column: Xbridge C8 (50×4.6 mm) 3.5 μm, Rt (min): 3.884; Area (%): 99.27.

### Step-2: 3-(4-(((1r,4r)-4-aminocyclohexyl)(isopentyl)amino)-1-oxoisoindolin-2-yl)piperidine-2,6-dione hydrochloride

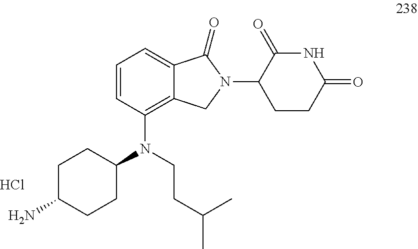 [SMILES: Cl ; CC(C)CCN(c1cccc2c1CN(C1CCC(=O)NC1=O)C2=O)[C@H]1CC[C@H](N)CC1]

To a stirred solution of tert-butyl ((1r,4r 4-((2-(2,6-dioxopiperidin-3-yl)-1-oxoisoindolin-4-yl) (isopentyl)amino) cyclohexyl) carbamate 4b (0.033 g, 0.063 mmol, 1 equiv)) in DCM (2 mL) was added HCl (4M in EtOAc, 0.486 mL, 1.942 mmol, 31 equiv) in drops at 0° C. The resulting solution was stirred for 3 h at room temperature. Upon completion of the reaction (as confirmed by TLC analysis, 100% EtOAc, R_{f}˜0.1) the reaction mixture was concentrated under vacuum to give a colorless gummy solid. The crude compound was triturated by MTBE (5 mL), the residue was dried under vacuum, dissolved in water (5 mL) and lyophilized to afford 3-(4-(((1r,4r)-4-aminocyclohexyl) (isopentyl)amino)-1-oxoisoindolin-2-yl) piperidine-2,6-dione hydrochloride 238 (0.021 g, 0.044 mmol, 70.9% yield) as off-white solid.

¹H-NMR (400 MHz, DMSO-d₆): 10.99 (s, 1H), 7.96 (s, 3H), 7.44-7.31 (m, 3H), 5.15-5.11 (m, 1H), 4.4-4.2 (m, 2H), 3.13 (br s, 2H), 2.97-2.93 (m, 3H), 2.68-2.51 (m, 2H), 2.01-1.96 (m, 3H), 1.91-1.80 (m, 2H), 1.61-1.51 (m, 3H), 1.38-1.34 (m, 2H), 1.13-1.12 (m, 2H), 0.83-0.81 (m, 6H).

LCMS: 427.3 (M+1), Method: Column: Atlantis dC18 (50×4.6 mm) 3.5 μm, Mobile Phase A: 0.1% TFA in water. Mobile Phase B: 0.1% TFA in ACN. Flow rate: 1.5 mL/min. Rt (min): 1.352; Area (%): 98.195.

HPLC Method: Mobile Phase A: 0.1% TFA in water. Mobile Phase B: ACN. Flow rate: 2.0 mL/min. Column: Xbridge C8(50×4.6 mm) 3.5 μm. Rt (min): 2.536; Area (%): 99.464.

### Hydrochloride Salt Form of Compound 239

### 3-(4-(((1r,4r)-4-aminocyclohexyl) (2-(piperidin-4-yl) ethyl) amino)-1-oxoisoindolin-2-yl) piperidine-2,6-dione, 2HCl

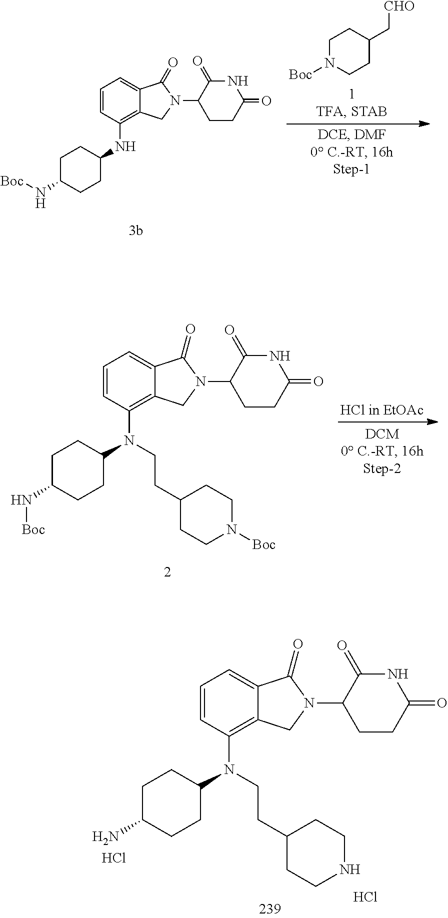

### Step-1: tert-butyl 4-(2-(((1r,4r)-4-((tert-butoxycarbonyl) amino) cyclohexyl) (2-(2,6-dioxopiperidin-3-yl)-1-oxoisoindolin-4-yl) amino) ethyl) piperidine-1-carboxylate

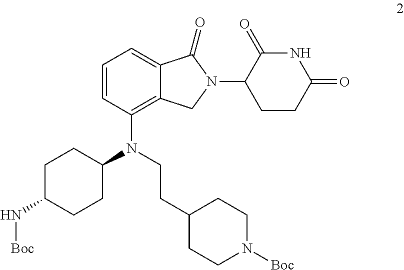 [SMILES: CC(C)(C)OC(=O)N[C@H]1CC[C@H](N(CCC2CCN(C(=O)OC(C)(C)C)CC2)c2cccc3c2CN(C2CCC(=O)NC2=O)C3=O)CC1]

To a stirred solution of tert-butyl ((1r,4r)-4-((2-(2,6-dioxopiperidin-3-yl)-1-oxoisoindolin-4-yl) amino) cyclohexyl) carbamate 3b (0.100 g, 0.219 mmol, 1 equiv) and tert-butyl 4-(2-oxoethyl) piperidine-1-carboxylate 1 (0.149 g, 0.657 mmol, 3 equiv) in a mixture of DCE (5 mL) and DMF (2 mL) were added trifluoroacetic acid (0.051 mL, 0.657 mmol, 3 equiv) followed by sodium triacetoxyborohydride (0.139 g, 0.657 mmol, 3 equiv) at 0° C. The resulted reaction mixture was stirred overnight at room temperature. Upon completion of the reaction (as confirmed by LCMS analysis) the reaction mixture was poured into ice cold water (15 mL) and extracted with DCM (3×10 mL). The combined organic layer was washed with brine (30 mL), dried over anhydrous sodium sulphate, filtered and concentrated to get the crude product as yellow gummy liquid. The obtained crude compound was purified by reverse phase column chromatography (Grace® column: C18 40 μm, 120 g; flow rate: 20 mL/min; 0.1% aqueous FA/ACN mobile phase, product eluted at 70% of ACN in 0.1% aqueous FA) to afford the desired product tert-butyl 4-(2-(((1r,4r)-4-((tert-butoxycarbonyl) amino) cyclohexyl) (2-(2,6-dioxopiperidin-3-yl)-1-oxoisoindolin-4-yl) amino) ethyl) piperidine-1-carboxylate 2 (0.089 g, 0.130 mmol, 59.3% yield) as off-white solid.

¹H-NMR (400 MHz, DMSO-d₆): δ 10.97 (s, 1H), 7.41 (d, J=7.60 Hz, 1H), 7.32-7.26 (m, 2H), 6.65 (d, J=6.80 Hz, 1H), 5.11 (q, J=5.20 Hz, 1H), 4.32 (d, J=17.20 Hz, 1H), 4.23 (d, J=17.20 Hz, 1H), 3.87 (d, J=10.40 Hz, 2H), 3.14 (t, J=7.20 Hz, 3H), 2.96 (d, J=2.80 Hz, 2H), 2.68-2.57 (m, 2H), 2.01 (t, J=5.20 Hz, 1H), 1.78-1.71 (m, 4H), 1.56 (t, J=11.60 Hz, 5H), 1.38 (t, J=1.60 Hz, 20H), 1.17 (t, J=6.00 Hz, 4H), 0.90 (t, J=3.20 Hz, 2H).

LCMS: 612.8 [M-(tert-Bu)]*. Method: Column: XBridge C8 (50×4.6 mm) 3.5 μm, Mobile phase A: 0.1% TFA in water, B: 0.1% TFA in ACN, Flow Rate: 1.5 mL/min, Rt (min): 2.062, Area (%): 96.460.

HPLC: Method: Mobile phase A: 0.1% TFA in water, B: ACN, Column: XBridge C8(50×4.6) mm, 3.5 m; Flow Rate: 2.0 mL/min; Rt (min): 4.105; Area (%): 97.417.

### Step-2: 3-(4-(((1r,4r)-4-aminocyclohexyl) (2-(piperidin-4-yl) ethyl) amino)-1-oxoisoindolin-2-yl) piperidine-2,6-dione, 2HCl

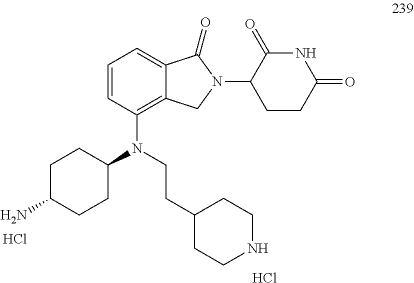 [SMILES: Cl ; N[C@H]1CC[C@H](N(CCC2CCNCC2)c2cccc3c2CN(C2CCC(=O)NC2=O)C3=O)CC1 ; Cl]

To a stirred solution of tert-butyl 4-(2-(((1r,4r)-4-((tert-butoxycarbonyl)amino)cyclohexyl)(2-(2,6-dioxopiperidin-3-yl)-1-oxoisoindolin-4-yl)amino)ethyl)piperidine-1-carboxylate 2 (0.085 g, 0.127 mmol, 1 equiv) in DCM (5 mL) was added 4N HCl in ethyl acetate (0.318 mL, 1.273 mmol, 10 equiv) at 0° C. The resultant reaction mixture was warmed to room temperature and stirred for 2 h. Upon completion of the reaction (as confirmed by TLC analysis, 100% EtOAc, R_{f} of the product˜0.1), the reaction mixture was concentrated under reduced pressure to get the crude product. The crude compound was purified by preparative HPLC (column: XSelect C18-250, 500 μl, Mobile phase: 0.1% HCl in water/MeCN, Flow-rate: 15 mL/min), pure fractions were collected and lyophilized to afford the desired product 3-(4-(((1r,4r)-4-aminocyclohexyl)(2-(piperidin-4-yl)ethyl)amino)-1-oxoisoindolin-2-yl)piperidine-2,6-dione, 2HCl 239 (0.060 g, 0.111 mmol, 87% yield) as off-white solid.

¹H-NMR (400 MHz, DMSO-d₆): δ 11.00 (s, 1H), 8.97 (d, J=10.00 Hz, 1H), 8.66 (d, J=8.80 Hz, 1H), 8.07 (s, 3H), 7.41 (d, J=42.00 Hz, 3H), 5.13 (d, J=5.20 Hz, 1H), 4.32 (dd, J=17.60, Hz, 2H), 3.17 (t, J=6.80 Hz, 4H), 2.98-2.89 (m, 3H), 2.79-2.71 (m, 2H), 2.68-2.60 (m, 2H), 1.99 (d, J=10.40 Hz, 3H), 1.80-1.71 (m, 4H), 1.54 (d, J=22.40 Hz, 3H), 1.38 (t, J=12.00 Hz, 2H), 1.29-1.20 (m, 4H).

LCMS: 468.2 (M+H), Method: Mobile phase A: 0.1% FA in water, Mobile phase B: ACN, Column: Atlantis dC18 (50×4.6 mm) 5 μm, Flow Rate: 1.5 mL/min, Rt (min): 0.939, Area (%): 99.380.

HPLC: Method: Mobile phase A: 0.10% TFA in water, Mobile phase B: MeOH, Column: Atlantis dC18 (250×4.6) mm, 5 μm, FlowRate: 1.0 mL/min, Rt (min): 7.569, Area (%): 99.071.

### Hydrochloride Salt Form of Compound 240

### 3-(4-(((1r,4r)-4-aminocyclohexyl)(2-(tetrahydro-2H-pyran-4-yl)ethyl)amino)-1-oxoisoindolin-2-yl)piperidine-2,6-dione hydrochloride

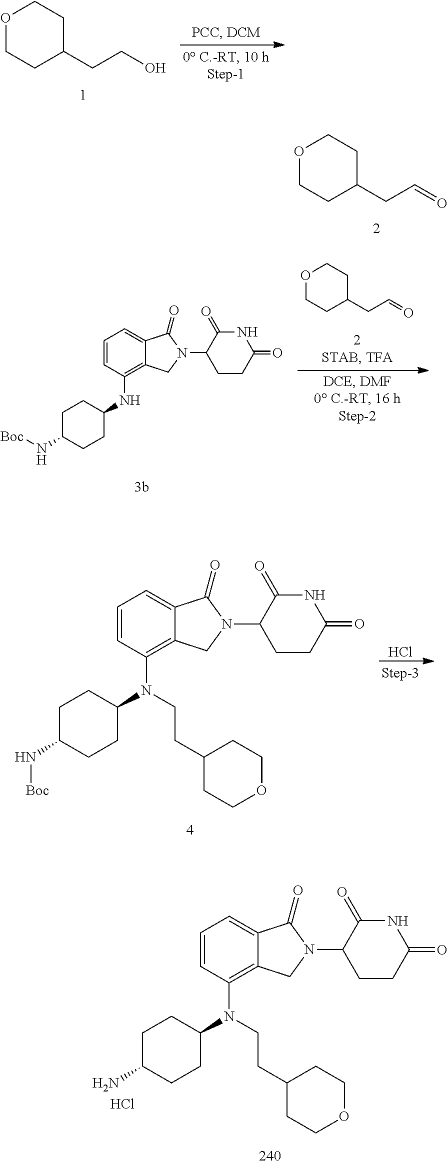

### Step-1:2-(tetrahydro-2H-pyran-4-yl)acetaldehyde

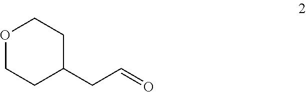

To a stirred solution of 2-(tetrahydro-2H-pyran-4-yl)ethan-1-ol 1 (2 g, 15.36 mmol, 1 equiv) in DCM (20 mL) was added PCC (6.62 g, 30.7 mmol, 2 equiv) portion wise at 0° C. The reaction mixture was warmed to room temperature and stirred for 10 h. Upon completion (as monitored by LCMS-ELSD), the reaction mixture was diluted with DCM (50 mL), adsorbed in silica gel (60-120 mesh, 15 g) and filtered through short silica bed. The silica bed was washed with excess of DCM (450 mL). The filtrate was concentrated under reduced pressure (at 30° C.) to afford the crude product 2-(tetrahydro-2H-pyran-4-yl)acetaldehyde 2 (0.65 g, 5.07 mmol, 33.0% yield) as brown gummy liquid.

¹H-NMR (400 MHz, DMSO-d₆): δ 9.68 (s, 1H), 3.83-3.78 (m, 2H), 3.34-3.27 (m, 2H), 2.38-2.36 (m, 2H), 2.00-2.09 (m, 1H), 1.57-1.53 (m, 2H), 1.28-1.23 (m, 2H).

### Step-2: tert-butyl ((1r,4r)-4-((2-(2,6-dioxopiperidin-3-yl)-1-oxoisoindolin-4-yl)(2-(tetrahydro-2H-pyran-4-yl)ethyl)amino)cyclohexyl)carbamate

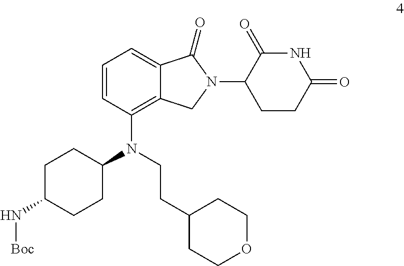 [SMILES: CC(C)(C)OC(=O)N[C@H]1CC[C@H](N(CCC2CCOCC2)c2cccc3c2CN(C2CCC(=O)NC2=O)C3=O)CC1]

To a stirred solution of tert-butyl ((1r,4r)-4-((2-(2,6-dioxopiperidin-3-yl)-1-oxoisoindolin-4-yl)amino)cyclohexyl)carbamate 3b (0.075 g, 0.164 mmol, 1 equiv) and 2-(tetrahydro-2H-pyran-4-yl)acetaldehyde 2 (0.032 g, 2.464 mmol, 15 equiv) in DCE (1.5 mL), DMF (1 mL) mixture was added TFA (0.094 g, 0.821 mmol, 5 equiv) followed by sodium triacetoxyborohydride (0.016 g, 0.756 mmol, 4.6 equiv) at 0° C. The reaction mixture was then stirred for overnight at room temperature. Upon completion of reaction (as confirmed by LCMS), the reaction was quenched with ice-water (20 mL) and extracted with DCM (2×30 mL). The combined organic layer was washed with brine (30 mL), dried over anhydrous sodium sulphate and concentrated under reduced pressure to get the crude compound. The crude compound was purified by preparative-HPLC (Column: X-Bridge-C18-150, Additional column: XSelect C18-19.1×150 mm Mobile phase: 0.1% TFA in water/ACN, Flow-rate: 15 mL/min) to afford the desired product tert-butyl ((1r,4r)-4-((2-(2,6-dioxopiperidin-3-yl)-1-oxoisoindolin-4-yl)(2-(tetrahydro-2H-pyran-4-yl)ethyl)amino)cyclohexyl)carbamate 4 (0.042 g, 0.071 mmol, 20.16% yield) as off-white solid.

¹H-NMR (400 MHz, DMSO-d₆): δ 10.98 (s, 1H), 7.43-7.27 (m, 3H), 6.68 (d, J=7.20 Hz, 1H), 5.11 (q, J=4.80, 1H), 4.33 (d, J=17.20 Hz, 1H), 4.23 (d, J=18.00 Hz, 1H), 3.79-3.76 (m, 2H), 3.29-3.15 (m, 5H), 2.96-2.87 (m, 2H), 2.51-2.50 (m, 2H), 2.02-2.00 (m, 1H), 1.81-1.72 (m, 3H), 1.57-1.47 (m, 6H), 1.37 (s, 9H), 1.10-1.04 (m, 6H).

LCMS: 569.3 (M+H). Method: Mobile Phase A: 0.1% FA in water. Mobile Phase B: ACN. Flow Rate:1.5 mL/min. Column: Atlantis dC18 (50×4.6 mm) 5 μm. Rt (min): 2.247; Area (%): 95.073.

HPLC Method: Mobile Phase A: 0.1% TFA in water. Mobile Phase B: ACN. Flow Rate: 2.0 mL/min. Column: Xbridge C8(50×4.6 mm) 3.5 μm, Rt (min): 3.381; Area (%): 96.011.

### Step-3: 3-(4-(((1r,4r)-4-aminocyclohexyl)(2-(tetrahydro-2H-pyran-4-yl)ethyl)amino)-1-oxoisoindolin-2-yl)piperidine-2,6-dione hydrochloride

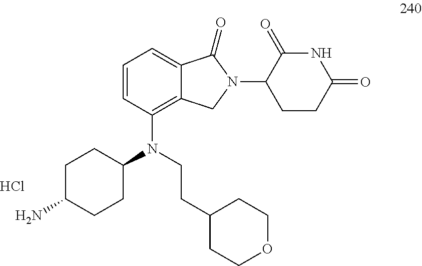 [SMILES: Cl ; N[C@H]1CC[C@H](N(CCC2CCOCC2)c2cccc3c2CN(C2CCC(=O)NC2=O)C3=O)CC1]

To a stirring solution of tert-butyl ((1r,4r)-4-((2-(2,6-dioxopiperidin-3-yl)-1-oxoisoindolin-4-yl)(2-(tetrahydro-2H-pyran-4-yl)ethyl)amino)cyclohexyl)carbamate 4 (0.042 g, 0.074 mmol, 1 equiv) in DCM (1.5 mL) was added HCl (4M solution. in EtOAc, 0.277 mL, 1.108 mmol, 15 equiv) at 0° C. The resulting solution was stirred at room temperature for 3 h. Upon completion of the reaction (as confirmed by TLC, Mobile phase: 100% EtOAc, R_{f}˜0.1), the reaction mixture was concentrated under vacuum to get the crude product as colorless gummy solid. The crude compound was triturated with MTBE (2×5 mL), and dried under reduced pressure to afford the desired product 3-(4-(((1r,4r)-4-aminocyclohexyl)(2-(tetrahydro-2H-pyran-4-yl)ethyl)amino)-1-oxoisoindolin-2-yl)piperidine-2,6-dione hydrochloride 240 (0.0091 mg, 0.018 mmol, 42.85% yield) as off-white solid.

¹H-NMR (400 MHz, DMSO-d₆): δ 10.95 (br s, 1H), 7.68 (br s, 3H), 7.46-7.42 (m, 1H), 7.34-7.28 (m, 2H), 5.13 (q, J=5.20 Hz, 1H), 4.33 (d, J=17.20 Hz, 1H), 4.23 (d, J=17.20 Hz, 1H), 3.79-3.77 (m, 2H), 3.13-3.32 (m, 4H), 2.97-2.91 (m, 3H), 2.62-2.53 (m, 2H), 1.96-1.93 (m, 3H), 1.80-1.77 (m, 2H), 1.58-1.46 (m, 5H), 1.18-1.17 (m, 2H), 1.10-1.06 (m, 4H).

LCMS: 469.4 (M+1), Method: Column: Atlantis DC18 (50×4.6) 3.5 μm, Mobile Phase A: 0.1% TFA in water. Mobile Phase B: ACN. Flow Rate:1.5 mL/min. Rt (min): 1.412; Area (%): 99.315.

HPLC Method: Mobile Phase A: 0.1% TFA in water. Mobile Phase B: ACN. Flow Rate: 2.0 mL/min. Column: Xbridge C8(50×4.6 mm), 3.5 μm. Rt (min): 2.078; Area (%): 99.586.

### Hydrochloride Salt form of Compound 241

### 3-(4-(((1r,4r)-4-aminocyclohexyl)(3-(piperidin-4-yl)propyl)amino)-1-oxoisoindolin-2-yl)piperidine-2,6-dione, 2HCl

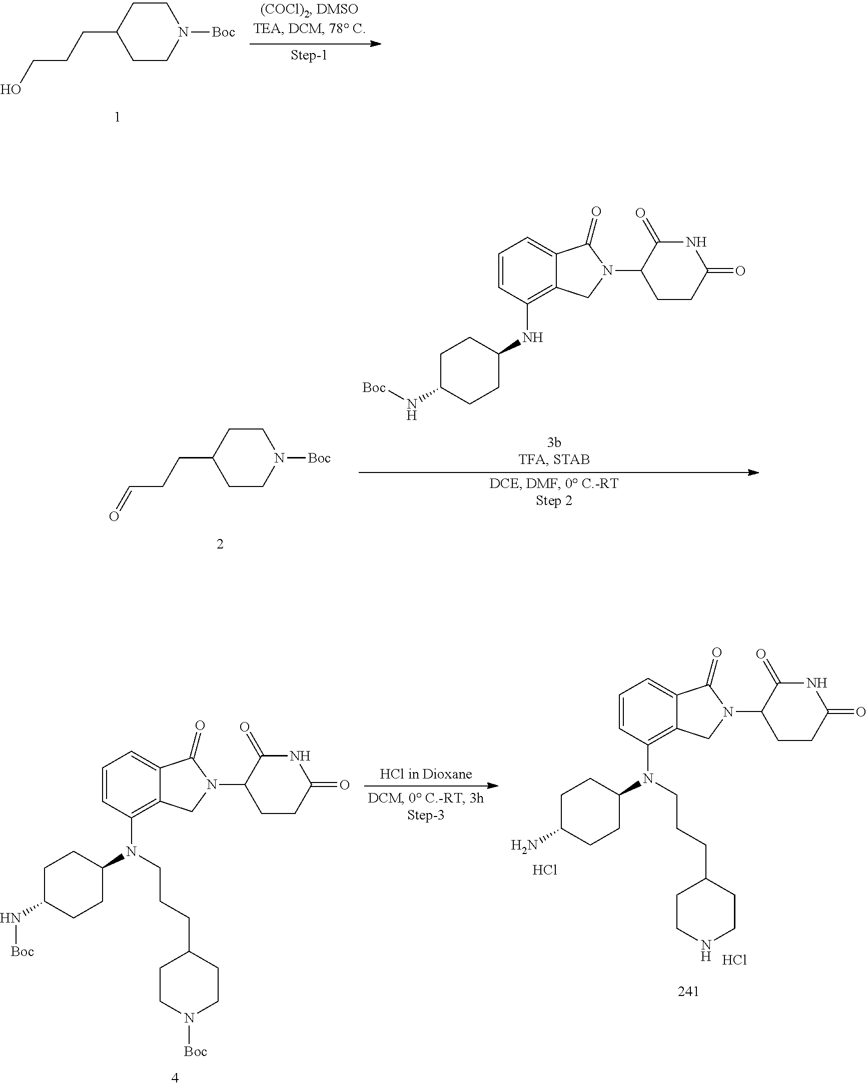

### Step-1: tert-butyl 4-(3-oxopropyl)piperidine-1-carboxylate

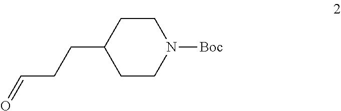 [SMILES: CC(C)(C)OC(=O)N1CCC(CCC=O)CC1]

To a stirred solution of oxalyl chloride (0.719 mL, 8.22 mmol, 2.0 equiv) in dry DCM (10.00 mL) at −78° C. under nitrogen atmosphere was added dropwise a solution of DMSO (1.166 mL, 16.44 mmol, 4.0 equiv) in DCM (3.00 mL). After 20 min, a solution of tert-butyl 4-(3-hydroxypropyl)piperidine-1-carboxylate 1 (1.0 g, 4.11 mmol, 1.0 equiv) in DCM (3.00 mL) was added dropwise over 5 min. After stirring for an additional 1 h at −78° C., triethylamine (3.44 mL, 24.66 mmol, 6.0 equiv) was added dropwise. After 45 min, the reaction became a thick white slurry. The reaction mixture was allowed to warm to room temperature and stirred for 1 h under nitrogen atmosphere. Upon completion of the reaction (as confirmed by TLC analysis, 50% EtOAc in pet ether, R_{f}˜0.6). The reaction mixture was quenched with ice cold 1N aqueous HCl solution (25 mL) at 0° C. and diluted with DCM (5 mL). The organic layer was separated and the aqueous layer was extracted with DCM (1×10 mL). The combined organic layer was washed with saturated aqueous NaHCO₃ solution (35 mL) followed by brine (50 mL), dried over anhydrous sodium sulphate, filtered and concentrated under reduced pressure at 35° C. to afford the crude of tert-butyl 4-(3-oxopropyl)piperidine-1-carboxylate 2 (0.950 g, 3.94 mmol, 96% yield) as clear oil which solidified in the freezer.

¹H-NMR (400 MHz, CDCl₃): δ 9.79 (t, J=1.60 Hz, 1H), 2.71-2.64 (m, 4H), 2.50-2.46 (m, 2H), 1.68-1.57 (m, 4H), 1.48-1.41 (m, 10H), 1.13-1.09 (m, 2H).

### Step-2: tert-butyl 4-(3-(((1r,4r)-4-((tert-butoxycarbonyl)amino)cyclohexyl)(2-(2,6-dioxopiperidin-3-yl)-1-oxoisoindolin-4-yl)amino)propyl)piperidine-1-carboxylate

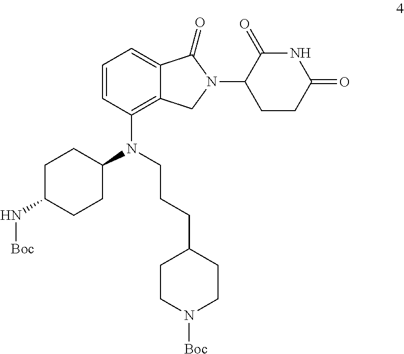 [SMILES: CC(C)(C)OC(=O)N[C@H]1CC[C@H](N(CCCC2CCN(C(=O)OC(C)(C)C)CC2)c2cccc3c2CN(C2CCC(=O)NC2=O)C3=O)CC1]

To a stirred solution of tert-butyl ((1r,4r)-4-((2-(2,6-dioxopiperidin-3-yl)-1-oxoisoindolin-4-yl)amino)cyclohexyl)carbamate 3b (0.1 g, 0.219 mmol, 1.0 equiv) and crude tert-butyl 4-(3-oxopropyl)piperidine-1-carboxylate 2 (0.264 g, 1.095 mmol, 5.0 equiv) in a mixture of DCE (5 mL) and DMF (2 mL), were added trifluoroacetic acid (0.075 g, 0.657 mmol, 3.0 equiv) and sodium triacetoxyborohydride (0.232 g, 1.095 mmol, 5.0 equiv) at 0° C. The resulting reaction mixture was stirred at room temperature overnight. Upon completion of the reaction (as confirmed by LCMS analysis) the reaction mixture was poured into ice cold water (15 mL) and extracted with EtOAc (3×20 mL). The combined organic layer was washed with brine (50 mL), dried over anhydrous sodium sulphate, filtered and concentrated to get the crude product. The crude compound was purified by reverse-phase column chromatography (Grace® column: C18 40 μm, 120 g; flow rate: 20 mL/min; 0.1% aqueous FA/ACN mobile phase, eluted with 60-70% of ACN in 0.1% aqueous FA) pure fractions were collected and lyophilized to afford the desired product tert-butyl 4-(3-(((1r,4r)-4-((tert-butoxycarbonyl)amino)cyclohexyl)(2-(2,6-dioxopiperidin-3-yl)-1-oxoisoindolin-4-yl)amino)propyl)piperidine-1-carboxylate 4 (0.130 g, 0.190 mmol, 87% yield) as off-white solid.

¹H-NMR (400 MHz, DMSO-d₆): δ 10.97 (s, 1H), 7.42 (t, J=7.60 Hz, 1H), 7.31-7.25 (m, 2H), 6.68 (d, J=7.60 Hz, 1H), 5.09-5.15 (m, 1H), 4.32 (d, J=17.20 Hz, 1H), 4.23 (d, J=17.20 Hz, 1H), 3.98-3.83 (m, 3H), 3.10-3.07 (m, 3H), 2.90 (m, 2H), 2.68-2.56 (m, 3H), 2.1-1.95 (m, 1H), 1.80-1.71 (m, 3H), 1.60-1.48 (m, 5H), 1.38 (d, J=8.40 Hz, 18H), 1.26-1.14 (m, 7H), 0.86 (m, 2H).

LCMS: 582.4 [(M+H) (-tert-Bu)], Method: Mobile phase A: 0.1% TFA in water, Mobile phase B: ACN, Column: XBridge C8 (50×4.6 mm) 3.5 μm, Flow Rate: 2.0 mL/min, Rt (min): 1.965, Area (%): 98.395.

HPLC: Method: Mobile phase A: 0.1% TFA in water, Mobile phase B: ACN, Column: Xbridge C8(50×4.6) mm, 3.5 m, Flow: 2.0 mL/min, Rt (min): 4.503, Area (%): 99.885.

### Step-3: 3-(4-(((1r,4r)-4-aminocyclohexyl)(3-(piperidin-4-yl)propyl)amino)-1-oxoisoindolin-2-yl)piperidine-2,6-dione, 2HCl

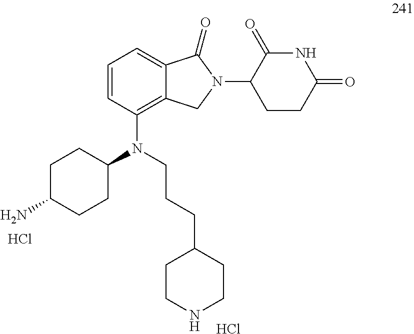 [SMILES: Cl ; N[C@H]1CC[C@H](N(CCCC2CCNCC2)c2cccc3c2CN(C2CCC(=O)NC2=O)C3=O)CC1 ; Cl]

To a stirred solution of tert-butyl 4-(3-(((1r,4r)-4-((tert-butoxycarbonyl)amino)cyclohexyl)(2-(2,6-dioxopiperidin-3-yl)-1-oxoisoindolin-4-yl)amino)propyl)piperidine-1-carboxylate 4 (0.125 g, 0.183 mmol, 1 equiv) in DCM (3 mL) was added HCl (4.0 M HCl in 1,4-dioxane, 0.917 mL, 3.67 mmol, 20 equiv) at 0° C. The resultant reaction mixture was allowed to warm to room temperature and stirred for 3 h. Upon completion of the reaction (as confirmed by LCMS analysis & TLC analysis, 100% EtOAc, Rf˜0.1), the reaction mixture was concentrated under reduced pressure to get the crude product as pale brown gummy solid. The obtained solid was washed with MTBE (10 mL), dried under vacuum and lyophilized. The obtained compound was impure so further purified by preparative HPLC (column: XBridge C18-250, injection volume-500 ul, Mobile phase A: 0.1% HCl in water/ACN, Flow-rate: 15 mL/min) to afford the desired product 3-(4-(((1r,4r)-4-aminocyclohexyl)(3-(piperidin-4-yl)propyl)amino)-1-oxoisoindolin-2-yl)piperidine-2,6-dione, 2HCl 241 (0.042 g, 0.076 mmol, 78% yield) as white solid.

¹H-NMR (400 MHz, DMSO-d₆): δ 10.99 (s, 1H), 8.80 (s, 1H), 8.54 (s, 1H), 8.01 (s, 3H), 7.45-7.21 (m, 3H), 5.13 (q, J=5.20 Hz, 1H), 4.45-4.15 (m, 2H), 3.17-2.89 (m, 7H), 2.77-2.71 (m, 2H), 2.68-2.64 (m, 1H), 2.51 (s, 1H), 1.99-1.97 (m, 3H), 1.79 (s, 2H), 1.66-1.63 (m, 4H), 1.38-1.36 (m, 3H), 1.23 (m, 6H).

LCMS: 482.3 (M+H), Method: Mobile phase A: 0.1% FA in water, Mobile phase B: ACN, Column: Atlantis dC18 (50×4.6 mm) 5 μm, Flow Rate: 1.5 ml/min Rt (min): 1.067, Area (%): 99.114.

HPLC: Method: Mobile phase A: 0.1% TFA in water, Mobile phase B: ACN, Column: X Bridge C8 (50×4.6) mm, 3.5 m, Flow Rate: 2.0 mL/min, Rt (min): 1.416, Area (%): 99.915.

### Hydrochloride Salt form of Compound 242

### 3-(4-(((1r,4r)-4-aminocyclohexyl)(3-(tetrahydro-2H-pyran-4-yl)propyl)amino)-1-oxoisoindolin-2-yl)piperidine-2,6-dione, HCl

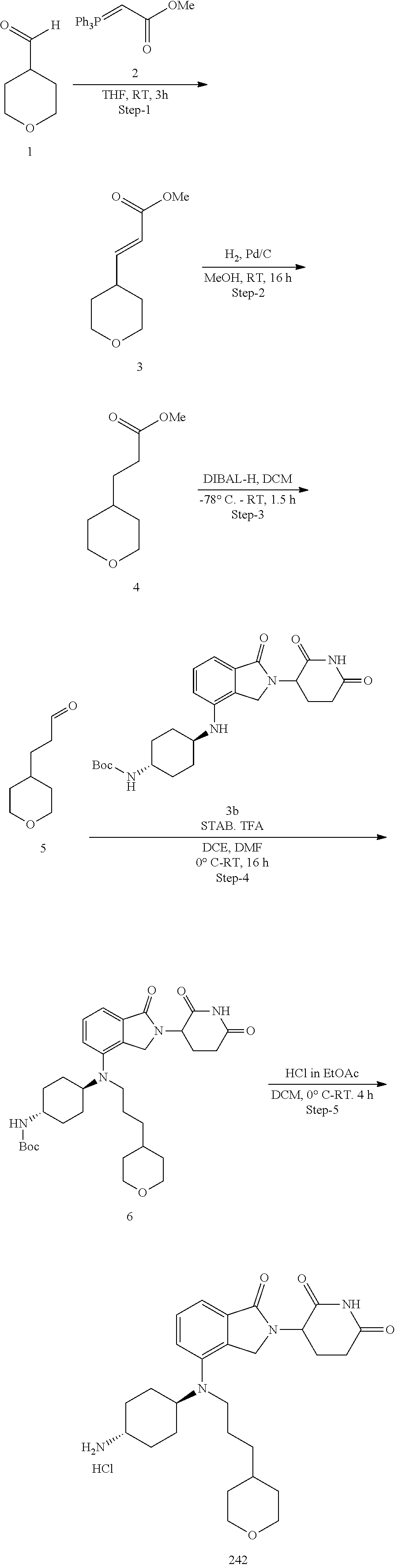

### Step-1: methyl (E)-3-(tetrahydro-2H-pyran-4-yl)acrylate

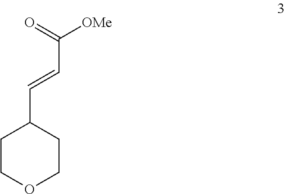 [SMILES: COC(=O)/C=C/C1CCOCC1]

To a stirred solution of tetrahydro-2H-pyran-4-carbaldehyde 1 (1.0 g, 8.76 mmol, 1.0 equiv) in dry THF (10 ml) was added methyl 2-(triphenyl-15-phosphanylidene)acetate 2 (3.22 g, 9.64 mmol, 1.1 equiv) at room temperature under nitrogen atmosphere. The mixture was stirred for 3 h at room temperature under nitrogen atmosphere. Upon completion of the reaction (as confirmed by TLC analysis, 20% EtOAc in pet ether, R_{f}˜0.8), the solvent was evaporated and hexane (50 ml) was added to the crude with stirring, the mixture was stirred for 1 h and filtered. The filtrate was concentrated under reduced pressure to get crude as colorless liquid. The crude compound was purified by purified by column chromatography (isolera, silica mesh size 230-400, flow rate 35 mL/min). The column was eluted using 30% EtOAc in pet ether to afford methyl (E)-3-(tetrahydro-2H-pyran-4-yl)acrylate 3 (1.25 g, 7.13 mmol, 81% yield) as colorless oil.

¹H-NMR (400 MHz, CDCl₃): δ 6.95-6.89 (m, 1H), 5.82 (dd, J=1.60, 15.60 Hz, 1H), 4.03-3.98 (m, 2H), 3.75 (s, 3H), 3.48-3.42 (m, 2H), 2.42-2.37 (m, 1H), 1.70-1.58 (m, 2H), 1.55-1.48 (m, 2H).

GCMS: 170.10 [M], Acq. method: HP5_LOW TEM FID.amx, Injection Volume: 1.0 pL, Rt (min): 6.524, Area (%): 97.08.

### Step-2: methyl 3-(tetrahydro-2H-pyran-4-yl)propanoate

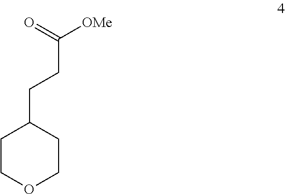 [SMILES: COC(=O)CCC1CCOCC1]

To a stirring solution of Methyl (E)-3-(tetrahydro-2H-pyran-4-yl)acrylate 3 (1.2 g, 7.05 mmol, 1.0 equiv) in MeOH (20.0 ml) was added Pd—C (10% wt/wt) (0.150 g, 0.141 mmol, 20 mol %) under nitrogen atmosphere and then the mixture was stirred under hydrogen atmosphere (Bladder pressure) for overnight. Upon completion of the reaction (as confirmed by TLC, mobile phase 20% EtOAc in pet ether, R_{f}˜0.3 & 1HNMR), The reaction mixture was filtered and washed with MeOH (2×10 mL) followed by water (1×5 mL) and concentrated under reduced pressure to afford crude product as methyl 3-(tetrahydro-2H-pyran-4-yl)propanoate 4 (0.94 g, 5.46 mmol, 77% yield) as pale yellow liquid. The crude product was used as such in next step with-out further purification.

¹H-NMR (400 MHz, CDCl₃): δ 3.99-3.95 (m, 2H), 3.69 (s, 3H), 3.41-3.34 (m, 2H), 2.36 (t, J=8.00 Hz, 2H), 1.64-1.59 (m, 4H), 1.54-1.48 (m, 1H), 1.35-1.24 (m, 2H).

### Step-3: 3-(tetrahydro-2H-pyran-4-yl)propanal

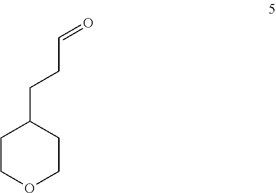

To a stirred solution of methyl 3-(tetrahydro-2H-pyran-4-yl)propanoate 4 (0.9 g, 5.23 mmol, 1.0 equiv) in dry DCM (10.00 mL) at −78° C. under nitrogen atmosphere was added DIBAL-H (1.0 M sol in hexane) (6.27 mL, 6.27 mmol, 1.2 equiv) dropwise under nitrogen atmosphere. The resulting solution was allowed to warm to room temperature and stirred for 1 h. TLC indicates that formation of aldehyde and presence of SM so the reaction mixture cooled to −78° C. and added DIBAL-H (1.0 M sol in hexane) (5.23 mL, 5.23 mmol, 1.0 equiv) as dropwise under nitrogen atmosphere. The resultant solution was allowed to warm to room temperature and stirred for 20 min. under nitrogen atmosphere. Upon completion of the reaction (as confirmed by TLC, mobile phase 30% EtOAc in pet ether, R_{f}˜0.2), the reaction mixture was quenched with saturated aqueous sodium potassium tartrate solution (15 mL) at 0° C., filtered through celite bed and wash the bed with DCM (1×25 mL). The organic layer was separated and the aqueous layer was extracted with DCM (2×30 mL). The combined organic layer was washed with brine (50 mL), dried over anhydrous sodium sulphate, filtered and concentrated under reduced pressure at 35° C. of rotavapor bath temperature to afford the crude product 3-(tetrahydro-2H-pyran-4-yl)propanal 5 (0.800 g, 5.63 mmol, 108% yield) as pale yellow gummy liquid. The crude product was used as such in next step with-out further purification.

¹H-NMR (400 MHz, CDCl₃): δ 9.80 (t, J=1.60 Hz, 1H), 3.99-3.95 (m, 2H), 3.37-3.38 (m, 2H), 2.50-2.46 (m, 2H), 1.64-1.60 (m, 4H), 1.59-1.58 (m, 1H), 1.28-1.32 (m, 2H).

### Step-4: tert-butyl ((1r,4r)-4-((2-(2,6-dioxopiperidin-3-yl)-1-oxoisoindolin-4-yl)(3-(tetrahydro-2H-pyran-4-yl)propyl)amino)cyclohexyl)carbamate

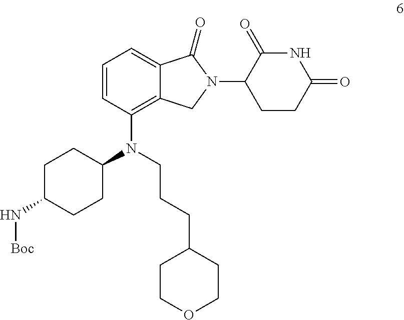 [SMILES: CC(C)(C)OC(=O)N[C@H]1CC[C@H](N(CCCC2CCOCC2)c2cccc3c2CN(C2CCC(=O)NC2=O)C3=O)CC1]

To a stirred solution of tert-butyl ((1r,4r)-4-((2-(2,6-dioxopiperidin-3-yl)-1-oxoisoindolin-4-yl)amino)cyclohexyl)carbamate 3b (0.1 g, 0.219 mmol, 1.0 equiv) and 3-(tetrahydro-2H-pyran-4-yl)propanal (0.623 g, 4.38 mmol, 10.0 equiv) in a mixture of DCE (4 mL) and DMF (2 mL) were added trifluoroacetic acid (0.101 mL, 1.314 mmol, 3.0 equiv) and sodium triacetoxyborohydride (0.464 g, 2.190 mmol, 5.0 equiv) at 0° C. The resulting reaction mixture was stirred at room temperature for overnight. Upon completion of the reaction (as confirmed by TLC analysis, 80% EtOAc in pet ether, R_{f}˜0.4 & LCMS analysis) the reaction mixture was poured into ice cold water (15 mL) and extracted with EtOAc (3×20 mL). The combined organic layer was washed with brine (50 mL), dried over anhydrous sodium sulphate, filtered and concentrated to get the crude product as yellow gummy solid. The crude compound was purified by reverse-phase column chromatography (Grace® column: C18-40 μm, 120 g; flow rate: 20 mL/min; 0.1% aqueous FA/ACN mobile phase, eluted with 60-70% of ACN in 0.1% of FA in water), pure fractions were collected and lyophilized to afford the desired product [tert-butyl ((1r,4r)-4-((2-(2,6-dioxopiperidin-3-yl)-1-oxoisoindolin-4-yl)(3-(tetrahydro-2H-pyran-4-yl)propyl)amino)cyclohexyl)carbamate 6 (0.16 g, 0.274 mmol, 62.6% yield)] as off-white solid.

¹H-NMR (400 MHz, DMSO-d₆): δ 10.97 (s, 1H), 7.42 (t, J=7.60 Hz, 1H), 7.31-7.25 (m, 2H), 6.68 (d, J=7.60 Hz, 1H), 5.11 (dd, J=4.80, 13.00 Hz, 1H), 4.30-4.21 (m, 2H), 3.77-3.74 (m, 2H), 3.32-3.16 (m, 5H), 3.09-3.08 (m, 2H), 2.57-2.51 (m, 1H), 2.04-1.98 (m, 1H), 1.81-1.72 (m, 4H), 1.53-1.41 (m, 2H), 1.37-1.34 (m, 12H), 1.24-1.14 (m, 6H), 1.04-1.00 (m, 2H).

LCMS: 583.3 (M+H), Method: Mobile phase A: 0.1% TFA in water, Mobile phase B: 0.1% TFA in ACN, Column: XBridge C8 (50×4.6 mm) 3.5 μm, Flow Rate: 1.5 ml/min, Rt (min): 1.644, Area (%): 98.639.

HPLC: Method: Mobile phase A: 0.1% TFA in water, Mobile phase B: ACN, Column: Xbridge C8(50×4.6) mm, 3.5 m, Flow: 2.0 mL/min, Rt (min): 3.403, Area (%): 99.947.

### Step-5: 3-(4-(((1r,4r)-4-aminocyclohexyl)(3-(tetrahydro-2H-pyran-4-yl)propyl)amino)-1-oxoisoindolin-2-yl)piperidine-2,6-dione, HCl

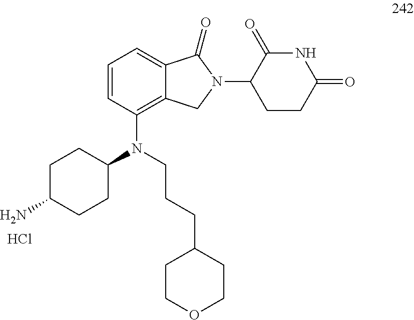 [SMILES: Cl ; N[C@H]1CC[C@H](N(CCCC2CCOCC2)c2cccc3c2CN(C2CCC(=O)NC2=O)C3=O)CC1]

To a stirred solution of tert-butyl ((1r,4r)-4-((2-(2,6-dioxopiperidin-3-yl)-1-oxoisoindolin-4-yl)(3-(tetrahydro-2H-pyran-4-yl)propyl)amino)cyclohexyl)carbamate 6 (0.26 g, 0.446 mmol, 1.0 equiv) in DCM (4 mL) was added hydrochloric acid (4.0 M solution in EtOAc, 2.231 mL, 8.92 mmol, 20 equiv) at 0° C. The resultant reaction mixture was allowed to warm to room temperature and stirred for 4 h. Upon completion of the reaction (as confirmed by LCMS & TLC, mobile phase 100% EtOAc, Rf˜0.1), the reaction mixture was concentrated under reduced pressure to get the crude product as pale yellow gummy solid. The obtained solid was washed with MTBE (2×10 mL), dried under reduced pressure and lyophilized to afford the desired product 3-(4-(((1r,4r)-4-aminocyclohexyl)(3-(tetrahydro-2H-pyran-4-yl)propyl)amino)-1-oxoisoindolin-2-yl)piperidine-2,6-dione hydrochloride 242 (0.216 g, 0.414 mmol, 93% yield)] as pale yellow solid.

¹H-NMR (400 MHz, DMSO-d₆): δ 11.00 (s, 1H), 7.95 (s, 3H), 7.37 (d, J=49.20 Hz, 3H), 5.16-5.11 (m, 1H), 4.04 (m, 2H), 3.78-3.74 (m, 2H), 3.22-2.90 (m, 7H), 2.63-2.51 (m, 2H), 1.98-1.92 (m, 3H), 1.78 (s, 2H), 1.58 (s, 2H), 1.44-0.15 (m, 8H), 1.07-0.99 (m, 2H).

LCMS: 483.2 (M+H), Method: Mobile phase A: 0.1% FA in water, Mobile phase B: ACN, Column: Atlantis dC18 (50×4.6 mm) 5 μm, Flow rate: 1.5 mL/min. Rt (min): 1.539, Area (%): 99.570.

HPLC: Method: Mobile phase A: 0.1% TFA in water, Mobile phase B: AeCN, Column: X Bridge C8 (50×4.6) mm, 3.5 μm, Flow rate: 2.0 mL/min, Rt (min): 2.128, Area (%): 99.511.

### Hydrochloride Salt Form of Compound 243

### 3-(4-(((1r,4r)-4-aminocyclohexyl)(2-(piperazin-1-yl)ethyl)amino)-1-oxoisoindolin-2-yl)piperidine-2,6-dione, 3HCl

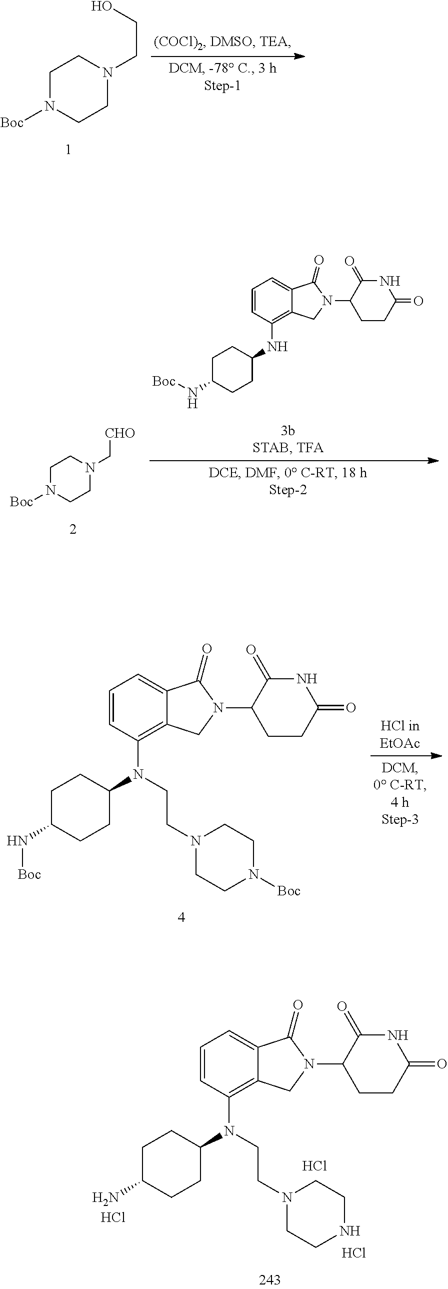

### Step-1: tert-butyl 4-(2-oxoethyl)piperazine-1-carboxylate

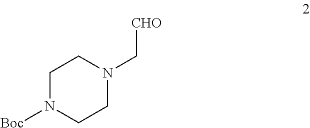 [SMILES: CC(C)(C)OC(=O)N1CCN(CC=O)CC1]

To a stirred solution of oxalyl chloride (4.47 mL, 52.1 mmol, 4.0 equiv) in DCM (40.0 mL) was added a solution of DMSO (7.40 mL, 104 mmol, 8.0 equiv) in DCM (6.0 mL) dropwise at −78° C. under nitrogen atmosphere. The solution was stirred for 30 min at −78° C. and a solution of tert-butyl 4-(2-hydroxyethyl)piperazine-1-carboxylate 1 (3.0 g, 13.03 mmol, 1.0 equiv) in DCM (6.0 mL) was added dropwise. The resulting solution was stirred 1.5 h at −78° C., then added triethylamine (4.85 mL, 34.8 mmol, 12.0 equiv) as dropwise at −78° C. Then the reaction mixture was slowly warmed to room temperature and stirred for 1 h. Upon completion of the reaction (as confirmed by TLC analysis, 10% MeOH in DCM, R_{f}˜0.4), the reaction mixture was quenched with saturated aq. solution of NaHCO₃ (25 mL) at 0° C. and diluted with DCM (25 mL). The organic layer was separated and the aqueous layer was extracted with DCM (1×40 mL). The combined organic layer was washed with brine (150 mL), dried over anhydrous sodium sulphate, filtered and concentrated under reduced pressure to afford the crude product tert-butyl 4-(2-oxoethyl)piperazine-1-carboxylate 2 (2.6 g, 11.39 mmol, 87% yield) as pale yellow sticky liquid, which was used in the next step without further purification.

¹H-NMR (400 MHz, CDCl₃): δ 9.73 (t, J=1.20 Hz, 1H), 3.51 (t, J=4.80 Hz, 4H), 3.22 (d, J=1.60 Hz, 2H), 2.50 (t, J=4.80 Hz, 4H), 1.48 (s, 9H).

### Step-2: tert-butyl 4-(2-(((1r,4r)-4-((tert-butoxycarbonyl)amino)cyclohexyl)(2-(2,6-dioxopiperidin-3-yl)-1-oxoisoindolin-4-yl)amino)ethyl)piperazine-1-carboxylate

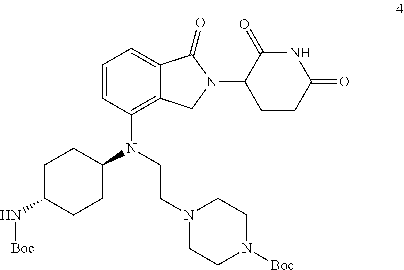 [SMILES: CC(C)(C)OC(=O)N[C@H]1CC[C@H](N(CCN2CCN(C(=O)OC(C)(C)C)CC2)c2cccc3c2CN(C2CCC(=O)NC2=O)C3=O)CC1]

To a stirred solution of tert-butyl ((1r,4r)-4-((2-(2,6-dioxopiperidin-3-yl)-1-oxoisoindolin-4-yl)amino)cyclohexyl)carbamate 3b (0.5 g, 1.095 mmol, 1 equiv) and tert-butyl 4-(2-oxoethyl)piperazine-1-carboxylate 2 (1.250 g, 5.48 mmol, 5 equiv) in DCE (8.0 mL), DMF (2.0 mL) were added trifluoroacetic acid (0.375 g, 3.29 mmol, 3.0 equiv) and sodium triacetoxyborohydride (1.161 g, 5.48 mmol, 5 equiv) at 0° C. The resulting reaction mixture was stirred at room temperature overnight. Upon completion of the reaction (as confirmed by LCMS analysis) the reaction mixture was poured into ice cold water (25 mL) and extracted with ethyl acetate (2×20 mL). The combined organic layer was washed with brine (30 mL), dried over anhydrous sodium sulphate, filtered and concentrated to get the crude product. The crude compound was purified by preparative HPLC (Column: XSelect C18-19.1×250 mm, Mobile phase: 10 mm ammonium aceturate in Water/ACN, Flow-rate: 15 mL/min), pure fractions were collected and lyophilized to get the desired product as yellow solid. The obtained solid was dissolved in DCM (15 mL) and washed with water (2×15 mL), dried over Na₂SO₄, filtered and concentrated under reduced pressure to afford desired product tert-butyl 4-(2-(((1r,4r)-4-((tert-butoxycarbonyl)amino)cyclohexyl)(2-(2,6-dioxopiperidin-3-yl)-1-oxoisoindolin-4-yl)amino)ethyl)piperazine-1-carboxylate 4 (0.150 g, 0.219 mmol, 20.02% yield) as yellow solid.

¹H-NMR (400 MHz, DMSO-d6): δ 10.97 (s, 1H), 7.42 (t, J=7.60 Hz, 1H), 7.32-7.30 (m, 2H), 6.69-6.67 (m, 1H), 5.11 (dd, J=5.20, 13.00 Hz, 1H), 4.38-4.24 (m, 2H), 3.27-3.22 (m, 7H), 2.99-2.91 (m, 2H), 2.68-2.61 (m, 2H), 2.34-2.18 (m, 6H), 2.02-1.99 (m, 1H), 1.81-1.72 (m, 4H), 1.53-1.50 (m, 2H), 1.42-1.37 (m, 20H), 1.24-1.17 (m, 2H).

LCMS: 669.4 (M+H). Method: Column: XBridge C8 (50×4.6 mm) 3.5 μm, Mobile phase A: 0.1% TFA in H₂O, B: 0.1% TFA in ACN, Flow Rate: 1.5 mL/min, Rt (min): 1.798, Area (%): 96.396.

HPLC: Method: Mobile phase A: 0.1% TFA in H₂O, B: ACN, Column: XBridge C8(50×4.6) mm, 3.5 m; Flow Rate: 2.0 mL/min; Rt (min): 3.793; Area (%): 97.740.

### Step-3:3-(4-(((1r,4r)-4-aminocyclohexyl)(2-(piperazin-1-yl)ethyl)amino)-1-oxoisoindolin-2-yl)piperidine-2,6-dione, 3HCl

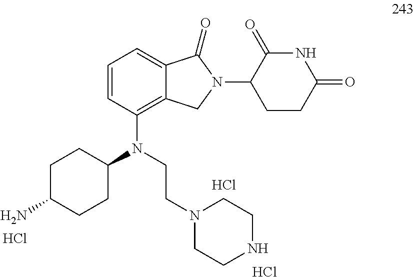 [SMILES: Cl ; N[C@H]1CC[C@H](N(CCN2CCNCC2)c2cccc3c2CN(C2CCC(=O)NC2=O)C3=O)CC1 ; Cl ; Cl]

To a stirred solution of tert-butyl 4-(2-(((1r,4r)-4-((tert-butoxycarbonyl)amino)cyclohexyl)(2-(2,6-dioxopiperidin-3-yl)-1-oxoisoindolin-4-yl)amino)ethyl)piperazine-1-carboxylate 4 (0.250 g, 0.374 mmol, 1 equiv) in DCM (5 mL) was added hydrochloric acid (4.0M sol in ethyl acetate, 0.093 mL, 0.374 mmol, 20.0 equiv) at 0° C. The resultant reaction mixture was allowed to stir at room temperature for 4 h. Upon completion of the reaction (as confirmed by LCMS analysis), the reaction mixture was concentrated under reduced pressure to get the crude product. The crude compound was washed by MTBE (2×10 mL), dried under vacuum, dissolved in 4 mL of ACN-water mixture (1:2) and lyophilized to get the desired product 3-(4-(((1r,4r)-4-aminocyclohexyl)(2-(piperazin-1-yl)ethyl)amino)-1-oxoisoindolin-2-yl)piperidine-2,6-dione, 3HCl 243 (0.171 g, 0.285 mmol, 76% yield) as pale brown solid.

¹H-NMR (400 MHz, DMSO-d₆): δ 12.23 (s, 1H), 11.01 (s, 1H), 9.77 (s, 2H), 8.04 (s, 3H), 7.48-7.41 (m, 3H), 5.16-5.11 (m, 1H), 4.44 (d, J=17.20 Hz, 1H), 4.27 (d, J=17.60 Hz, 1H), 3.67-3.64 (m, 3H), 3.45-3.33 (m, 7H), 3.04-2.95 (m, 3H), 2.94-2.89 (m, 2H), 2.68-2.67 (m, 2H), 2.00-1.97 (m, 3H), 1.83 (s, 2H), 1.65-1.56 (m, 2H), 1.39-1.35 (m, 2H).

LCMS: 469.2 (M+H). Method: Column: Atlantis dC18 (50×4.6) 5μ, Mobile phase A: 0.1% TFA in H₂O, B: MeOH, Flow Rate:0.7 mL/min, Rt (min): 2.925, Area (%): 99.339.

HPLC: Method: Mobile phase A: 0.1% TFA in H₂O, B: MeOH, Column: Atlantis dC18 (50×4.6 mm) 5 μm; Flow Rate: 1.0 mL/min; Rt (min): 6.640; Area (%): 96.230.

### Hydrochloride Salt Form of Compound 244

### 3 3-(4-(((1r,4r)-4-aminocyclohexyl)(2-morpholinoethyl)amino)-1-oxoisoindolin-2-yl)piperidine-2,6-dione, 2HCl

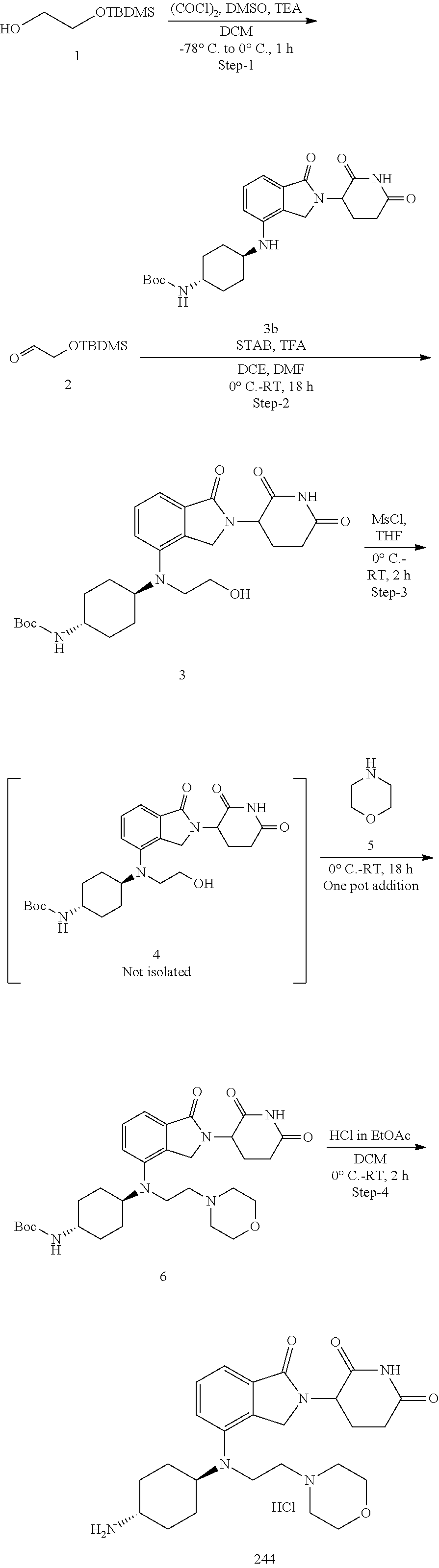

### Step-1:2-((tert-butyldimethylsilyl)oxy)acetaldehyde

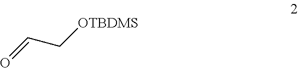 [SMILES: CC(C)(C)[Si](C)(C)OCC=O]

To a stirred solution of oxalyl chloride (0.645 mL, 7.37 mmol, 1.3 equiv) in DCM (10.0 mL) was added a solution of DMSO (1.046 mL, 14.74 mmol, 2.6 equiv) in DCM (1.5 mL) dropwise at −78° C. under nitrogen atmosphere. The solution was stirred for 15 min at −78° C. and a solution of 2-((tert-butyldimethylsilyl)oxy)ethan-1-ol 1 (1.0 g, 5.67 mmol, 1.0 equiv) in DCM (1.5 mL) was added dropwise. The resulting solution was stirred 30 min at −78° C., then added triethylamine (4.11 mL, 29.5 mmol, 5.2 equiv) as dropwise at −78° C. Then the reaction mixture was slowly warmed to 0° C. and stirred for 15 min. Upon completion of the reaction (as confirmed by TLC, mobile phase 20% EtOAc in pet ether, R_{f}˜0.4, KMnO₄ stain), the reaction mixture was quenched with ice cold water (15 mL) at 0° C. and diluted with DCM (15 mL). The organic layer was separated and the aqueous layer was extracted with DCM (2×10 mL). The combined organic layer was washed with saturated aqueous citric acid solution (1×70 mL) followed by saturated aqueous solution of NaHCO₃ (1×70 mL), brine (50 mL), dried over anhydrous sodium sulphate, filtered and concentrated under reduced pressure to afford the crude product 2-((tert-butyldimethylsilyl)oxy)acetaldehyde 2 (1.4 g, 8.03 mmol, 142% yield) as yellow liquid, which was used in the next step without further purification.

¹H-NMR (400 MHz, CDCl₃): δ 9.72 (t, J=0.80 Hz, 1H), 4.24 (d, J=0.80 Hz, 2H), 0.95 (s, 9H), 0.13 (s, 6H).

### Step-2: tert-butyl ((1r,4r)-4-((2-(2,6-dioxopiperidin-3-yl)-1-oxoisoindolin-4-yl)(2-hydroxyethyl)amino)cyclohexyl)carbamate

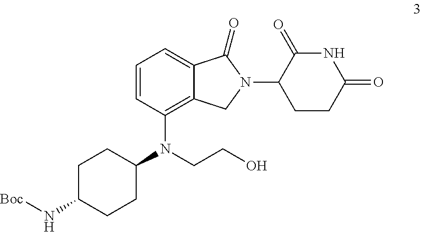 [SMILES: CC(C)(C)OC(=O)N[C@H]1CC[C@H](N(CCO)c2cccc3c2CN(C2CCC(=O)NC2=O)C3=O)CC1]

To a stirred solution of tert-butyl ((1r,4r)-4-((2-(2,6-dioxopiperidin-3-yl)-1-oxoisoindolin-4-yl)amino)cyclohexyl)carbamate 3b (0.2 g, 0.438 mmol, 1 equiv) and 2-((tert-butyldimethylsilyl)oxy)acetaldehyde 2 (0.764 g, 4.38 mmol, 10 equiv) in DCE (3.2 mL) and DMF (1.2 mL) were added trifluoroacetic acid (0.101 mL, 1.314 mmol, 3.0 equiv) and sodium triacetoxyborohydride (0.464 g, 2.190 mmol, 5 equiv) at 0° C. The resulting reaction mixture was stirred at room temperature for overnight. Upon completion of the reaction (as confirmed by TLC, mobile phase 100% EtOAc, R_{f}-0.3 & LCMS analysis) the reaction mixture was concentrated to get the crude product. The crude compound was purified by reverse-phase column chromatography (Grace® column: C18 40 μm, 120 g; flow rate: 20 mL/min; 0.1% FA in water/ACN mobile phase, eluted with 30-40% of ACN in 0.1% aqueous FA), pure fractions were collected and lyophilized to get the desired product tert-butyl ((1r,4r)-4-((2-(2,6-dioxopiperidin-3-yl)-1-oxoisoindolin-4-yl)(2-hydroxyethyl)amino)cyclohexyl)carbamate 3 (0.175 g, 0.346 mmol, 79% yield) as off-white solid.

¹H-NMR (400 MHz, DMSO-d₆): δ 10.97 (s, 1H), 7.41 (t, J=7.60 Hz, 1H), 7.30-7.27 (m, 2H), 6.69 (d, J=7.20 Hz, 1H), 5.12-5.08 (m, 1H), 4.45-4.40 (m, 1H), 4.36-4.24 (m, 2H), 3.27-3.22 (m, 5H), 3.21-3.17 (m, 1H), 3.03-2.91 (m, 1H), 2.62-2.53 (m, 1H), 2.03-2.00 (m, 1H), 1.81-1.73 (m, 4H), 1.50-1.47 (m, 2H), 1.40 (m, 9H), 1.37-1.18 (m, 2H).

LCMS: 501.3 (M+H). Method: Column: Atlantis dC18 (50×4.6 mm) 5 μm, Mobile phase A: 0.1% FA in water, B: ACN, Flow Rate: 1.5 mL/min, Rt (min): 1.894, Area (%): 97.451.

HPLC: Method info A: 0.1% TFA in water, B: ACN, Column: XBridge C8(50×4.6) mm, 3.5 μm; Flow Rate: 2.0 mL/min; Rt (min): 2.753; Area (%): 98.928.

### Step-3: tert-butyl ((1r,4r)-4-((2-(2,6-dioxopiperidin-3-yl)-1-oxoisoindolin-4-yl)(2-morpholinoethyl)amino)cyclohexyl)carbamate

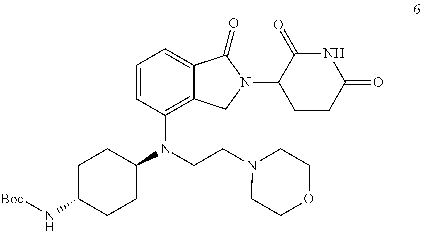 [SMILES: CC(C)(C)OC(=O)N[C@H]1CC[C@H](N(CCN2CCOCC2)c2cccc3c2CN(C2CCC(=O)NC2=O)C3=O)CC1]

To a stirred solution of tert-butyl ((1r,4r)-4-((2-(2,6-dioxopiperidin-3-yl)-1-oxoisoindolin-4-yl)(2-hydroxyethyl)amino)cyclohexyl)carbamate 3 (0.125 g, 0.250 mmol, 1 equiv) in dry THF (1.4 mL) was added triethylamine (0.045 mL, 0.325 mmol, 1.3 equiv) followed by a solution of mesyl-Cl (0.034 g, 0.300 mmol, 1.2 equiv) in THF (0.400 mL) as dropwise at 0° C. under nitrogen atmosphere and stirred for 30 min. The solution was warmed to room temperature and stirred for additional 1 h. Upon completion of the mesylation reaction (as confirmed by TLC, mobile phase 100% EtOAc, R_{f}˜0.5), the reaction mixture was again cooled to 0° C. and morpholine 5 (0.087 g, 0.999 mmol, 4.0 equiv) was added to the same reaction vessel and stirred for 30 min. The resulting reaction mixture was warmed to room temperature and stirred for overnight. Upon completion of the reaction (as confirmed by LCMS analysis) the reaction mixture was poured into ice cold water (15 mL) and extracted with EtOAc (3×20 mL). The combined organic layer was washed with brine (50 mL), dried over anhydrous sodium sulphate, filtered and concentrated to get the crude product. The crude compound was purified by preparative HPLC (column: XSelect C18-19.1×150 mm, 500 μl, Mobile phase: 0.1% TFA in water/ACN, Flow-rate: 15 mL/min), pure fractions were collected and lyophilized to afford the desired product tert-butyl ((1r,4r)-4-((2-(2,6-dioxopiperidin-3-yl)-1-oxoisoindolin-4-yl)(2-morpholinoethyl)amino)cyclohexyl)carbamate 6 (0.05 g, 0.085 mmol, 34.0% yield) as white solid.

¹H-NMR (400 MHz, DMSO-d₆): δ 11.01 (s, 1H), 9.64 (br s, 1H), 7.52-7.40 (m, 3H), 6.74 (d, J=7.60 Hz, 1H), 5.15-5.11 (m, 1H), 4.36 (d, J=17.20 Hz, 1H), 4.25 (d, J=17.60 Hz, 1H), 3.95-3.94 (m, 2H), 3.52-3.48 (m, 4H), 3.03-2.93 (m, 8H), 2.68-2.64 (m, 2H), 2.02-2.01 (m, 1H), 1.79-1.73 (m, 4H), 1.54-1.51 (m, 2H), 1.40-1.37 (m, 9H), 1.21-1.19 (m, 2H).

LCMS: 570.4 (M+H). Method: Column: Atlantis dC18 (50×4.6 mm) 5 μm, Mobile phase A: 0.1% FA in water, B: ACN, Flow Rate: 1.5 mL/min, Rt (min): 2.263, Area (%): 96.157.

HPLC: Method: Mobile phase A: 0.1% TFA in water, B: ACN, Column: XBridge C8(50×4.6) mm, 3.5 m; Flow Rate: 2.0 mL/min; Rt (min): 3.092; Area (%): 96.827.

### Step-4: 3-(4-(((1r,4r)-4-aminocyclohexyl)(2-morpholinoethyl)amino)-1-oxoisoindolin-2-yl)piperidine-2,6-dione, 2HCl

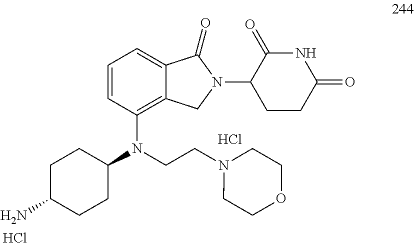 [SMILES: N[C@H]1CC[C@H](N(CCN2CCOCC2)c2cccc3c2CN(C2CCC(=O)NC2=O)C3=O)CC1 ; Cl ; Cl]

To a stirred solution of tert-butyl ((1r,4r)-4-((2-(2,6-dioxopiperidin-3-yl)-1-oxoisoindolin-4-yl)(2-morpholinoethyl)amino)cyclohexyl)carbamate 6 (0.06 g, 0.105 mmol, 1 equiv) in DCM (3.0 mL) was added hydrochloric acid (4M sol in ethyl acetate) (0.527 mL, 2.106 mmol, 20.0 equiv) at 0° C. The resultant reaction mixture was allowed to stir at room temperature for 2 h. Upon completion of the reaction (as confirmed by TLC, 100% EtOAc, R_{f}˜0.1 & LCMS analysis) the reaction mixture was concentrated under reduced pressure to get the crude product. The crude compound was washed with MTBE (2×10 mL), dried under vacuum, dissolved in water (3 mL) and lyophilized to afford the desired product 3-(4-(((1r,4r)-4-aminocyclohexyl)(2-morpholinoethyl)amino)-1-oxoisoindolin-2-yl)piperidine-2,6-dione, 2HCl 244 (0.057 g, 0.100 mmol, 95% yield) as pale yellow solid.

¹H-NMR (400 MHz, DMSO-d₆): δ 11.51 (s, 1H), 10.99 (s, 1H), 7.98 (s, 3H), 7.50-7.41 (m, 3H), 5.16-5.11 (m, 1H), 4.42 (d, J=17.20 Hz, 1H), 4.26 (d, J=17.20 Hz, 1H), 3.92-3.84 (m, 4H), 3.67-3.63 (m, 3H), 3.43 (s, 2H), 3.03-2.89 (m, 7H), 2.68-2.59 (m, 1H), 2.00-1.97 (m, 3H), 1.82-1.79 (m, 2H), 1.62-1.59 (m, 2H), 1.37 (m, 2H).

LCMS: 470.2 (M+H). Method: Column: XBridge C8 (50×4.6 mm) 3.5 μm, Mobile phase A: 0.1% TFA in water, B: ACN, Flow Rate: 0.7 mL/min, Rt (min): 2.374, Area (%): 95.077.

HPLC: Method: Mobile phase A: 0.1% TFA in water, B: ACN, Column: XBridge C8 (50×4.6 mm) 3.5 m; Flow Rate: 2.0 mL/min; Rt (min): 1.344; Area (%): 95.367.

### Hydrochloride Salt Form of Compound 245

### 3-(4-(((1r,4r)-4-aminocyclohexyl)(3-(piperazin-1-yl)propyl)amino)-1-oxoisoindolin-2-yl)piperidine-2,6-dione, 3HCl

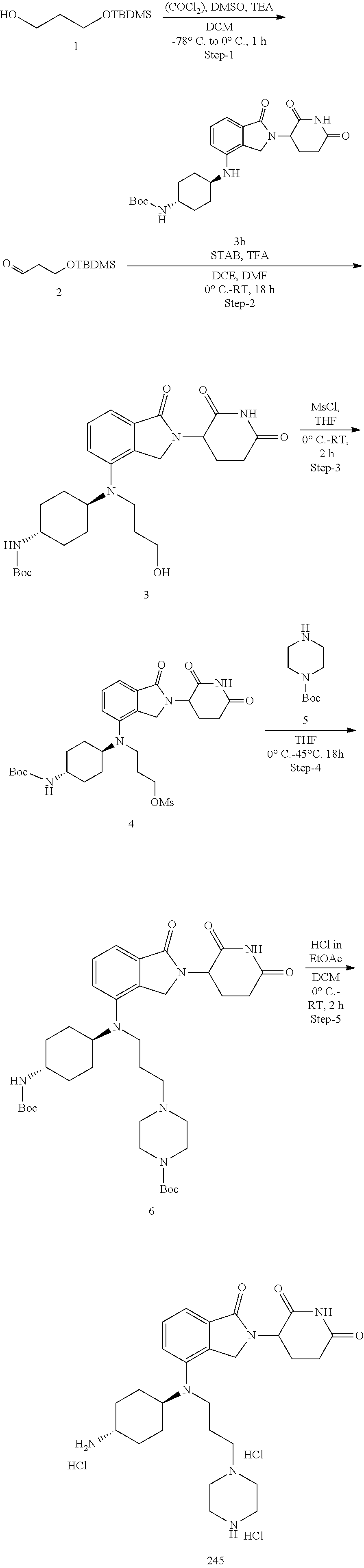

### Step-1: 3-((tert-butyldimethylsilyl)oxy)propanal

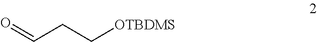 [SMILES: CC(C)(C)[Si](C)(C)OCCC=O]

To a stirred solution of oxalyl chloride (1.839 mL, 21.01 mmol, 2.0 equiv) in DCM (15.0 mL) was added a solution of DMSO (2.98 mL, 42.0 mmol, 4.0 equiv) in DCM (5.0 mL) dropwise at −78° C. under nitrogen atmosphere. The solution was stirred for 15 min at −78° C. and a solution of 3-((tert-butyldimethylsilyl)oxy)propan-1-ol 1 (2.0 g, 10.51 mmol, 1.0 equiv) in DCM (5.0 mL) was added dropwise. The resulting solution was stirred for 30 min at −78° C., then added triethylamine (8.79 mL, 63.0 mmol, 8.0 equiv) as dropwise at −78° C. Then the reaction mixture was slowly warmed to 0° C. and stirred for additional 15 min. Upon completion of the reaction (as confirmed by TLC, 30% EtOAc in pet ether, R_{f}˜0.4, KMnO₄ Stain), the reaction mixture was quenched with ice cold water (15 mL) at 0° C. and diluted with DCM (15 mL). The organic layer was separated and the aqueous layer was extracted with DCM (2×10 mL). The combined organic layer was washed with saturated aqueous citric acid solution (1×70 mL), saturated aqueous solution of NaHCO₃ (1×70 mL), brine (50 mL), dried over anhydrous sodium sulphate, filtered and concentrated under reduced pressure to afford the crude of 3-((tert-butyldimethylsilyl)oxy)propanal 2 (1.92 g, 10.19 mmol, 97% yield) as colourless liquid, which was used in the next step without further purification.

¹H-NMR (400 MHz, CDCl₃): δ 9.82 (t, J=2.40 Hz, 1H), 4.01 (t, J=6.00 Hz, 2H), 2.68-2.60 (m, 2H), 0.94-0.91 (m, 9H), 0.12-0.07 (m, 6H).

### Step-2: tert-butyl ((1r,4r)-4-((2-(2,6-dioxopiperidin-3-yl)-1-oxoisoindolin-4-yl)(3-hydroxypropyl)amino)cyclohexyl)carbamate

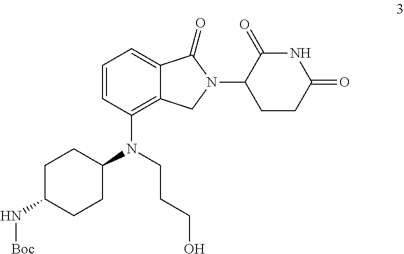 [SMILES: CC(C)(C)OC(=O)N[C@H]1CC[C@H](N(CCCO)c2cccc3c2CN(C2CCC(=O)NC2=O)C3=O)CC1]

To a stirred solution of tert-butyl ((1r,4r)-4-((2-(2,6-dioxopiperidin-3-yl)-1-oxoisoindolin-4-yl)amino)cyclohexyl)carbamate 3b (0.5 g, 1.095 mmol, 1 equiv) and 3-((tert-butyldimethylsilyl)oxy)propanal 2 (2.063 g, 10.95 mmol, 10 equiv) in DCE (3.2 mL), DMF (1.2 mL) were added trifluoroacetic acid (0.253 mL, 3.29 mmol, 3.0 equiv) and sodium triacetoxyborohydride (1.161 g, 5.48 mmol, 5 equiv) at 0° C. The resulting reaction mixture was stirred at room temperature for overnight. Upon completion of the reaction (as confirmed by TLC, 100% EtOAc, R_{f}˜0.3 & LCMS analysis) the reaction mixture was concentrated under reduced pressure to get the crude product. The crude compound was purified by reverse-phase column chromatography (Grace® column: C18 40 μm, 120 g; flow rate: 20 mL/min; 0.1% aqueous FA/ACN mobile phase, eluted with 30-40% of ACN in 0.1% aqueous FA) pure fractions were collected and lyophilized to get the desired product tert-butyl ((1r,4r)-4-((2-(2,6-dioxopiperidin-3-yl)-1-oxoisoindolin-4-yl)(3-hydroxypropyl)amino)cyclohexyl)carbamate 3 (0.295 g, 0.490 mmol, 44.7% yield) as pale yellow solid.

¹H-NMR (400 MHz, DMSO-d₆): δ 10.98 (s, 1H), 7.42 (t, J=7.60 Hz, 1H), 7.31-7.26 (m, 2H), 6.70 (d, J=7.60 Hz, 1H), 5.13-5.08 (m, 1H), 4.37-4.22 (m, 3H), 3.20-3.16 (m, 3H), 3.01-2.90 (m, 2H), 2.62-2.55 (m, 2H), 2.03-2.01 (m, 1H), 1.81-1.72 (m, 4H), 1.57-1.51 (m, 3H), 1.41-1.37 (m, 11H), 1.23-1.17 (m, 2H).

LCMS: 515.3 (M+H). Method: Column: Atlantis dC18 (50×4.6 mm) 5 μm, Mobile phase A: 0.1% FA in water, B: ACN, Flow Rate: 1.5 mL/min, Rt (min): 1.862, Area (%): 78.914.

HPLC: Method: Mobile phase A: 0.1% TFA in water, B: ACN, Column: XBridge C8(50×4.6) mm, 3.5 m; Flow Rate: 2.0 mL/min; Rt (min): 2.891; Area (%): 85.496.

### Step-3:3-(((1r,4r)-4-((tert-butoxycarbonyl)amino)cyclohexyl)(2-(2,6-dioxopiperidin-3-yl)-1-oxoisoindolin-4-yl)amino)propyl methanesulfonate

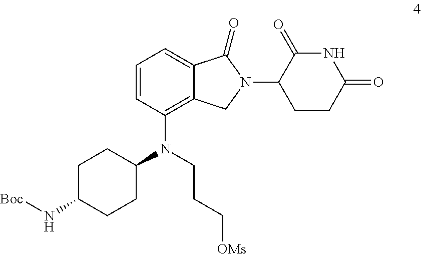 [SMILES: CC(C)(C)OC(=O)N[C@H]1CC[C@H](N(CCCOS(C)(=O)=O)c2cccc3c2CN(C2CCC(=O)NC2=O)C3=O)CC1]

To a stirred solution of tert-butyl ((1r,4r)-4-((2-(2,6-dioxopiperidin-3-yl)-1-oxoisoindolin-4-yl)(3-hydroxypropyl)amino)cyclohexyl)carbamate 3 (0.09 g, 0.175 mmol, 1 equiv) in dry THF (2.0 mL) was added triethylamine (0.026 g, 0.227 mmol, 1.3 equiv) followed by a solution mesyl-Cl (0.024 g, 0.210 mmol, 1.2 equiv) in THF (0.400 mL) as dropwise at 0° C. under nitrogen atmosphere and stirred for 30 min. Then the solution was warmed to room temperature and stirred for additional 1 h. Upon completion of the reaction (as confirmed by TLC, 100% EtOAc, R_{f}˜0.5), the reaction mixture was poured into ice cold water (15 mL) and extracted with EtOAc (3×25 mL). The combined organic layer was washed with brine (50 mL), dried over anhydrous sodium sulphate, filtered and concentrated to get the crude product 3-(((1r,4r)-4-((tert-butoxycarbonyl)amino)cyclohexyl)(2-(2,6-dioxopiperidin-3-yl)-1-oxoisoindolin-4-yl)amino)propyl methanesulfonate 4 (0.101 g, 0.131 mmol, 74.8% yield) as pale yellow gummy solid. The crude product was used as such in next step with-out further purification.

LCMS: 593.3 (M+H). Method: Column: XBridge C8 (50×4.6 mm) 3.5 μm, Mobile phase A: 0.1% TFA in H₂O, B: 0.1% TFA in ACN, Flow Rate: 1.5 mL/min, Rt (min): 1.830, Area (%): 76.783.

### Step-4: tert-butyl 4-(3-(((1r,4r)-4-((tert-butoxycarbonyl)amino)cyclohexyl)(2-(2,6-dioxopiperidin-3-yl)-1-oxoisoindolin-4-yl)amino)propyl)piperazine-1-carboxylate

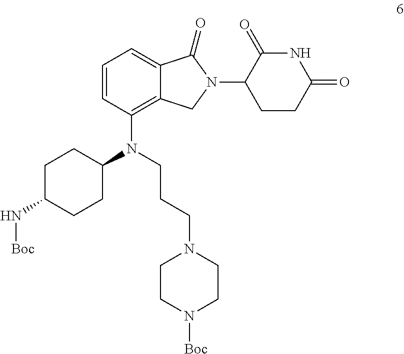 [SMILES: CC(C)(C)OC(=O)N[C@H]1CC[C@H](N(CCCN2CCN(C(=O)OC(C)(C)C)CC2)c2cccc3c2CN(C2CCC(=O)NC2=O)C3=O)CC1]

To a stirred solution of 3-(((1r,4r)-4-((tert-butoxycarbonyl)amino)cyclohexyl)(2-(2,6-dioxopiperidin-3-yl)-1-oxoisoindolin-4-yl)amino)propyl methanesulfonate 4 (0.102 g, 0.172 mmol, 1 equiv) in dry THF (2.0 mL) was added tert-butyl piperazine-1-carboxylate 5 (0.038 g, 0.207 mmol, 1.2 equiv) followed by N,N-diisopropylethylamine (0.036 mL, 0.207 mmol) at 0° C. under nitrogen atmosphere and stirred for 30 min. Then the resulting solution was heated to 45° C. for overnight. Upon completion of the reaction (as confirmed by LCMS analysis), the reaction mixture was poured into ice cold water (15 mL) and extracted with EtOAc (3×20 mL). The combined organic layer was washed with brine (50 mL), dried over anhydrous sodium sulphate, filtered and concentrated to get the crude product. The crude compound was purified by preparative HPLC (column: XSelect C18-250, 500 uL, Mobile phase: 0.1% FA in water/ACN, Flow-rate: 15 mL/min), pure fractions were collected and lyophilized to afford the desired product tert-butyl 4-(3-(((1r,4r)-4-((tert-butoxycarbonyl)amino)cyclohexyl)(2-(2,6-dioxopiperidin-3-yl)-1-oxoisoindolin-4-yl)amino)propyl)piperazine-1-carboxylate 6 (0.045 g, 0.065 mmol, 37.7% yield) as white solid.

¹H-NMR (400 MHz, DMSO-d₆): δ 10.99 (s, 1H), 8.33 (s, 1H), 7.42 (t, J=7.60 Hz, 1H), 7.30 (t, J=10.00 Hz, 2H), 6.70 (d, J=8.00 Hz, 1H), 5.13-5.09 (m, 1H), 4.35-4.22 (m, 2H), 3.25-3.24 (m, 4H), 3.17-3.14 (m, 1H), 2.99-2.91 (m, 2H), 2.61-2.60 (m, 4H), 2.52-2.51 (m, 2H), 2.24-2.19 (m, 6H), 2.01-1.99 (m, 1H), 1.81-1.72 (m, 4H), 1.54-1.45 (m, 2H), 1.42-1.37 (m, 18H), 1.22-1.16 (m, 2H).

LCMS: 683.4 (M+H). Method: Column: Atlantis dC18 (50×4.6) 5 μm, Mobile phase A: 0.1% FA in water, B: ACN, Flow Rate: 1.5 mL/min, Rt (min): 1.894, Area (%): 97.151.

HPLC: Method: Mobile phase A: 0.1% TFA in water, B: ACN, Column: XBridge C8(50×4.6) mm, 3.5 m; Flow Rate: 2.0 mL/min; Rt (min): 3.762; Area (%): 98.542.

### Step-5: 3-(4-(((1r,4r)-4-aminocyclohexyl)(3-(piperazin-1-yl)propyl)amino)-1-oxoisoindolin-2-yl)piperidine-2,6-dione, 3HCl

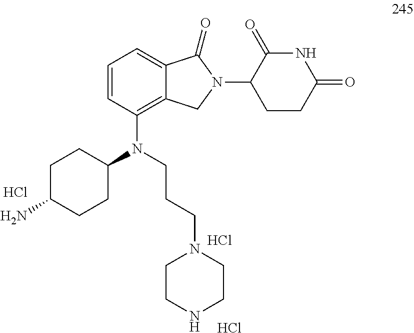 [SMILES: Cl ; N[C@H]1CC[C@H](N(CCCN2CCNCC2)c2cccc3c2CN(C2CCC(=O)NC2=O)C3=O)CC1 ; Cl ; Cl]

To a stirred solution of tert-butyl 4-(3-(((1r,4r)-4-((tert-butoxycarbonyl)amino)cyclohexyl)(2-(2,6-dioxopiperidin-3-yl)-1-oxoisoindolin-4-yl)amino)propyl)piperazine-1-carboxylate 6 (0.04 g, 0.059 mmol, 1 equiv) in DCM (2.2 mL) was added hydrochloric acid (4M sol in ethyl acetate) (0.293 mL, 1.172 mmol, 20.0 equiv) at 0° C. The resultant reaction mixture was allowed to stir at room temperature for 2 h. Upon completion of the reaction (as confirmed by TLC, 100% EtOAc, R_{f}˜0.1 & LCMS analysis), the reaction mixture was concentrated under reduced pressure to get the crude product. The crude compound was washed by MTBE (2×4 mL), dried under vacuum, dissolved in water (3 mL) and lyophilized to afford the desired product 3-(4-(((1r,4r)-4-aminocyclohexyl)(3-(piperazin-1-yl)propyl)amino)-1-oxoisoindolin-2-yl)piperidine-2,6-dione, 3HCl 245 (0.035 g, 0.058 mmol, 99% yield) as pale yellow solid.

¹H-NMR (400 MHz, DMSO-d₆): δ 11.72 (s, 1H), 10.98 (s, 1H), 9.62 (br s, 4H), 8.04-8.02 (m, 3H), 7.48-7.37 (m, 3H), 5.15-5.10 (m, 1H), 4.33 (d, J=31.60 Hz, 1H), 4.25 (d, J=Hz, 1H), 3.63-3.62 (m, 1H), 3.59-3.44 (m, 4H), 3.41 (s, 4H), 3.41-3.22 (m, 2H), 3.22-2.90 (m, 5H), 2.68-2.64 (m, 1H), 2.05-1.97 (m, 2H), 1.83-1.80 (m, 2H), 1.68-1.56 (m, 5H), 1.39-1.36 (m, 2H).

LCMS: 483.4 (M+H). Method: Column: XBridge C8 (50×4.6 mm) 3.5 μm, Mobile phase A: 0.1% TFA in water, B: TFA in ACN, Flow Rate: 1.5 mL/min, Rt (min): 0.829, Area (%): 99.572.

HPLC: Method: Mobile phase A: 0.1% TFA in water, B: ACN, Column: XBridge C8 (50×4.6 mm) 3.5 m; Flow Rate: 2.0 mL/min; Rt (min): 1.299; Area (%): 98.442.

### Hydrochloride Salt Form of Compound 246

### 3-(4-(((1r,4r)-4-aminocyclohexyl)(3-morpholinopropyl)amino)-1-oxoisoindolin-2-yl)piperidine-2,6-dione dihydrochloride

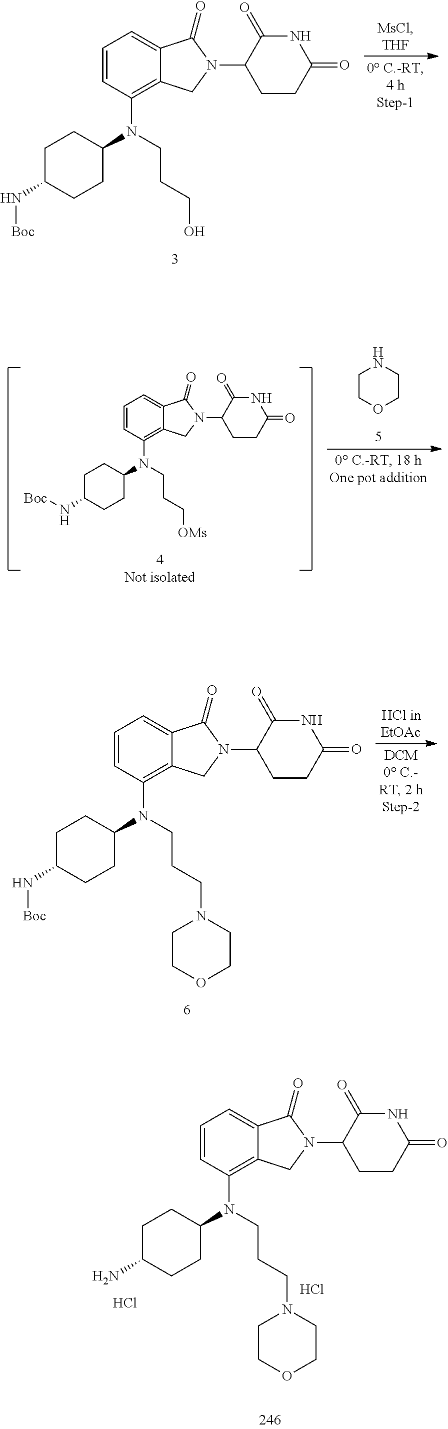

### Step-1: tert-butyl ((1r,4r)-4-((2-(2,6-dioxopiperidin-3-yl)-1-oxoisoindolin-4-yl)(3-morpholinopropyl)amino)cyclohexyl)carbamate

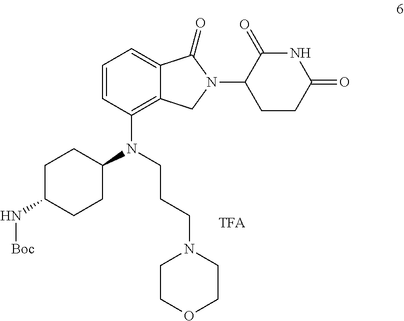 [SMILES: CC(C)(C)OC(=O)N[C@H]1CC[C@H](N(CCCN2CCOCC2)c2cccc3c2CN(C2CCC(=O)NC2=O)C3=O)CC1]

To a stirred solution of tert-butyl ((1r,4r)-4-((2-(2,6-dioxopiperidin-3-yl)-1-oxoisoindolin-4-yl)(3-hydroxypropyl)amino)cyclohexyl)carbamate 3 (0.1 g, 0.194 mmol, 1 equiv) in dry THF (1.4 ml) was added triethylamine (0.026 g, 0.253 mmol, 1.3 equiv) followed by a solution mesyl-Cl (0.027 g, 0.233 mmol, 1.2 equiv) in THF (0.400 mL) as dropwise at 0° C. under nitrogen atmosphere and stirred for 30 min. The solution was warmed to room temperature and stirred for additional 4 h. Upon completion of the mesylation reaction (as confirmed by TLC, 100% EtOAc, R_{f}˜0.5), the reaction mixture was again cooled to 0° C. and morpholine 5 (0.068 g, 0.777 mmol, 4.0 equiv) was added to the same reaction vessel and stirred for 30 min. The resulting reaction mixture was warmed to room temperature and stirred for overnight. Upon completion of the reaction (as confirmed by LCMS analysis) the reaction mixture was poured into ice cold water (15 mL) and extracted with EtOAc (3×20 mL). The combined organic layer was washed with brine (50 mL), dried over anhydrous sodium sulphate, filtered and concentrated to get the crude product. The crude compound was purified by preparative HPLC (column: XSelect C18-19.1×150 mm, 500 μl, Mobile phase: 0.1% TFA in water/ACN, Flow-rate: 15 mL/min), pure fractions were collected and lyophilized to afford the desired product tert-butyl ((1r,4r)-4-((2-(2,6-dioxopiperidin-3-yl)-1-oxoisoindolin-4-yl)(3-morpholinopropyl)amino)cyclohexyl)carbamate, TFA salt 6 (0.035 g, 0.048 mmol, 24.63% yield) as pale beige solid.

¹H-NMR (400 MHz, DMSO-d₆): δ 11.01 (s, 1H), 9.37-9.35 (m, 1H), 7.48-7.45 (m, 1H), 7.41-7.35 (m, 2H), 6.72 (d, J=8.00 Hz, 1H), 5.15-5.10 (m, 1H), 4.36 (d, J=17.20 Hz, 1H), 4.26 (d, J=17.60 Hz, 1H), 3.95-3.92 (m, 2H), 3.57-3.51 (m, 2H), 3.34-3.32 (m, 2H), 3.19-3.16 (m, 3H), 3.07-3.06 (m, 2H), 2.99-2.93 (m, 4H), 2.03-2.01 (m, 1H), 1.81-1.73 (m, 5H), 1.62-1.49 (m, 5H), 1.37 (s, 9H), 1.24-1.17 (m, 2H).

LCMS: 584.3 (M+H). Method: Column: XBridge C8(50×4.6) mm, 3.5 μm, Mobile phase A: 0.1% TFA in water, B: ACN, Flow Rate: 2.0 mL/min, Rt (min): 1.344, Area (%): 94.49.

HPLC: Method: Mobile phase A: 0.1% TFA in water, B: ACN, Column: XBridge C8(50×4.6) mm, 3.5 m; Flow Rate: 2.0 mL/min; Rt (min): 3.134; Area (%): 95.399.

### Step-2: 3-(4-(((1r,4r)-4-aminocyclohexyl)(3-morpholinopropyl)amino)-1-oxoisoindolin-2-yl)piperidine-2,6-dione, 2HCl

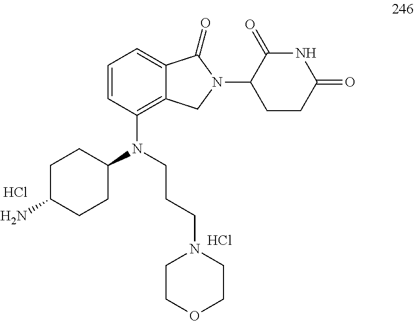 [SMILES: Cl ; N[C@H]1CC[C@H](N(CCCN2CCOCC2)c2cccc3c2CN(C2CCC(=O)NC2=O)C3=O)CC1 ; Cl]

To a stirred solution of tert-butyl ((1r,4r)-4-((2-(2,6-dioxopiperidin-3-yl)-1-oxoisoindolin-4-yl)(3-morpholinopropyl)amino)cyclohexyl)carbamate. TFA 6 (0.033 g, 0.049 mmol, 1 equiv) in DCM (2.2 mL) was added hydrochloric acid (4M sol in EtOAc) (0.283 mL, 1.131 mmol, 23.0 equiv) at 0° C. The resultant reaction mixture was allowed to stir at room temperature for 2 h. Upon completion of the reaction (as confirmed by TLC, 100% EtOAc, R_{f}˜0.1 & LCMS analysis), the reaction mixture was concentrated under reduced pressure to get the crude product. The crude compound was washed with MTBE (2×10 mL), dried under vacuum, dissolved in 3 mL of water and lyophilized to afford the desired product 3-(4-(((1r,4r)-4-aminocyclohexyl)(3-morpholinopropyl)amino)-1-oxoisoindolin-2-yl)piperidine-2,6-dione, 2HCl 246 (0.026 g, 0.046 mmol, 93.88% yield) as pale yellow solid.

¹H-NMR (400 MHz, DMSO-d₆): δ 11.00 (s, 1H), 10.82 (s, 1H), 8.01 (s, 3H), 7.47 (t, J=7.20 Hz, 2H), 7.39 (d, J=6.80 Hz, 1H), 5.15-5.10 (m, 1H), 4.41-4.24 (m, 2H), 3.91-3.88 (m, 2H), 3.76-3.70 (m, 2H), 3.31-3.29 (m, 2H), 3.27-3.21 (m, 2H), 3.02-2.90 (m, 7H), 2.51-2.50 (m, 2H), 2.09-1.96 (m, 3H), 1.82-1.79 (m, 2H), 1.67-1.52 (m, 2H), 1.41-1.35 (m, 2H), 1.32-1.20 (m, 2H).

LCMS: 484.3 (M+H). Method: Column: Atlantis dC18 (50×4.6 mm) 5 μm, Mobile phase A: 0.1% FA in water, B: ACN, Flow Rate: 1.5 mL/min, Rt (min): 0.937, Area (%): 98.122.

HPLC: Method: Mobile phase A: 0.1% TFA in water, B: ACN, Column: XBridge C8 (50×4.6 mm) 3.5 m; Flow Rate: 2.0 mL/min; Rt (min): 1.427; Area (%): 98.040.

### Hydrochloride Salt Form of Compound 247

### 3-(4-(((1r,4r)-4-aminocyclohexyl)(phenethyl)amino)-1-oxoisoindolin-2-yl)piperidine-2,6-dione, HCl

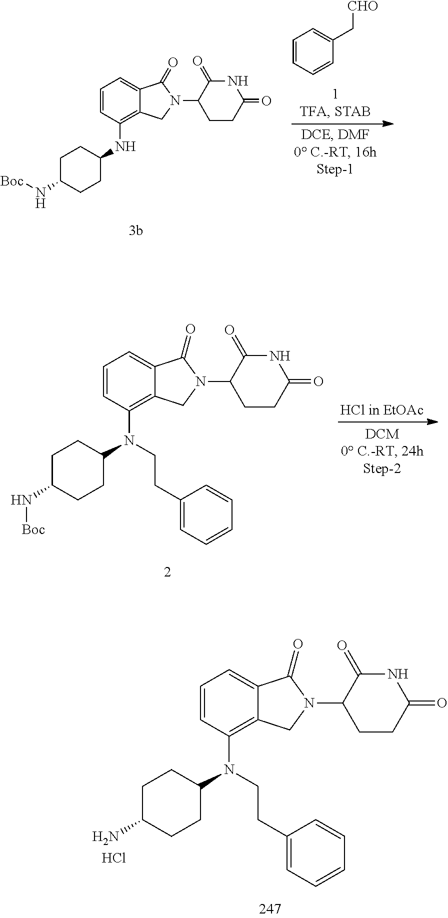

### Step-1: tert-butyl ((1r,4r)-4-((2-(2,6-dioxopiperidin-3-yl)-1-oxoisoindolin-4-yl)(phenethyl)amino)cyclohexyl)carbamate

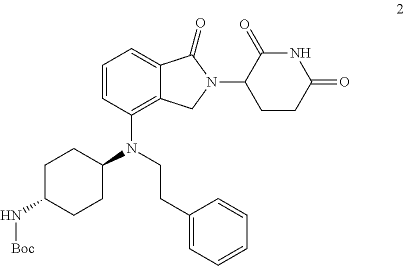 [SMILES: CC(C)(C)OC(=O)N[C@H]1CC[C@H](N(CCc2ccccc2)c2cccc3c2CN(C2CCC(=O)NC2=O)C3=O)CC1]

To a stirring solution of tert-butyl ((1r,4r)-4-((2-(2,6-dioxopiperidin-3-yl)-1-oxoisoindolin-4-yl)amino)cyclohexyl)carbamate 3b (0.099 g, 0.217 mmol, 1 equiv), 2-phenylacetaldehyde 1 (0.078 g, 0.651 mmol, 3 equiv) in a mixture of DCE (5 mL) and DMF (2 mL) were added trifluoroacetic acid (0.050 mL, 0.651 mmol, 3 equiv) followed by sodium triacetoxyborohydride (0.138 g, 0.651 mmol, 3 equiv) at 0° C. The resulted reaction mixture was stirred overnight at room temperature. Upon completion of the reaction (as confirmed by LCMS analysis), the reaction mixture was poured into ice cold water (15 mL) and extracted with DCM (3×10 mL). The combined organic layer was washed with brine (30 mL), dried over anhydrous sodium sulphate, filtered and concentrated to get the crude product as yellow gummy liquid. The obtained crude compound was purified by reverse phase column chromatography (Grace® column: C18 40 μm, 120 g; flow rate: 20 mL/min; 0.1% aqueous FA/ACN mobile phase, product eluted at 64% of ACN in 0.1% aqueous FA) to afford the desired product tert-butyl ((1r,4r)-4-((2-(2,6-dioxopiperidin-3-yl)-1-oxoisoindolin-4-yl) (phenethyl)amino) cyclohexyl) carbamate 2 (0.085 g, 0.152 mmol, 69.9% yield) as off-white solid.

¹H-NMR (400 MHz, DMSO-d₆): δ 10.97 (s, 1H), 7.48 (t, J=7.60 Hz, 1H), 7.39-7.34 (m, 2H), 7.24 (t, J=7.60 Hz, 2H), 7.16 (t, J=7.20 Hz, 1H), 7.05 (d, J=7.20 Hz, 2H), 6.65 (d, J=6.80 Hz, 1H), 5.13-5.09 (m, 1H), 4.15 (d, J=5.20 Hz, 2H), 3.39-3.32 (m, 2H), 3.3-3.1 (m, 1H), 2.97-2.88 (m, 2H), 2.68-2.54 (m, 2H), 2.46-2.39 (m, 2H), S 2.00-1.98 (m, 1H), 1.77-1.74 (m, 2H), 1.61-1.59 (m, 2H), 1.49-1.43 (m, 2H), 1.36 (s, 9H), 1.20-1.14 (m, 2H).

LCMS: 561.3 (M+H). Method: Column: XBridge C8 (50×4.6 mm) 3.5 μm, Mobile phase A: 0.1% TFA in water, B: 0.1% TFA in ACN, Flow Rate: 1.5 mL/min, Rt (min): 2.108, Area (%): 94.554.

HPLC: Method info A: 0.1% TFA in water, B: ACN, Column: XBridge C8(50×4.6 mm) 3.5 μm; Flow Rate: 2.0 mL/min; Rt (min): 4.417; Area (%): 99.950.

### Step-2: 3-(4-(((1r,4r)-4-aminocyclohexyl) (phenethyl)amino)-1-oxoisoindolin-2-yl) piperidine-2,6-dione, HCl

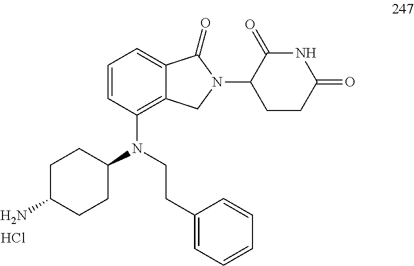 [SMILES: Cl ; N[C@H]1CC[C@H](N(CCc2ccccc2)c2cccc3c2CN(C2CCC(=O)NC2=O)C3=O)CC1]

To a stirred solution of tert-butyl ((1r,4r)-4-((2-(2,6-dioxopiperidin-3-yl)-1-oxoisoindolin-4-yl) (phenethyl)amino) cyclohexyl) carbamate 2 (0.08 g, 0.143 mmol, 1 equiv) in DCM (5 mL) was added 4N HCl in EtOAc (0.357 mL, 1.427 mmol, 10 equiv) at 0° C. The resultant reaction mixture was allowed to warm to room temperature and stirred for 24 h. Upon completion of the reaction (as confirmed by TLC analysis, 100% EtOAc, Rf˜0.1), the reaction mixture was concentrated under reduced pressure to get the crude product. The crude compound was purified by preparative HPLC (column: XSelect C18-250, 500 μl, Mobile phase: 0.1% HCl in water/ACN, Flow-rate: 15 mL/min), pure fractions were collected and lyophilized to afford the desired product 3-(4-(((1r,4r)-4-aminocyclohexyl) (phenethyl)amino)-1-oxoisoindolin-2-yl) piperidine-2,6-dione, HCl 247 (0.040 g, 0.080 mmol, 56.2% yield) as off-white solid.

¹H-NMR (400 MHz, DMSO-d₆): δ 10.99 (s, 1H), 7.93 (s, 3H), 7.46 (t, J=37.60 Hz, 3H), 7.25 (t, J=7.20 Hz, 2H), 7.19-7.15 (m, 1H), 7.06 (d, J=7.20 Hz, 2H), 5.16-5.11 (m, 1H), 4.4-4.1 (m, 2H), 3.55-3.35 (m, 2H), 3.02-2.88 (m, 3H), 2.68-2.58 (m, 2H), 2.50-2.39 (m, 2H), 2.02-1.92 (m, 3H), 1.68 (s, 2H), 1.52 (t, J=9.20 Hz, 2H), 1.37-1.29 (m, 2H).

LCMS: 461.3 (M+H), Method: Mobile phase A: 0.1% FA in water, Mobile phase B: ACN, Column: Atlantis dC18 (50×4.6 mm) 5 μm, Flow Rate: 1.5 ml/min, Rt (min): 2.108, Area (%): 99.919.

HPLC: Method: Mobile phase A: 0.1% TFA in water, Mobile phase B: ACN, Column: Atlantis dC18 (250×4.6 mm), 5 μm, Flow Rate:1.0 mL/min, Rt (min): 9.401, Area (%): 99.612.

### Hydrochloride Salt Form of Compound 251

### 3-(4-(((1r,4r)-4-aminocyclohexyl)(3-phenylpropyl)amino)-1-oxoisoindolin-2-yl)piperidine-2,6-dione hydrochloride

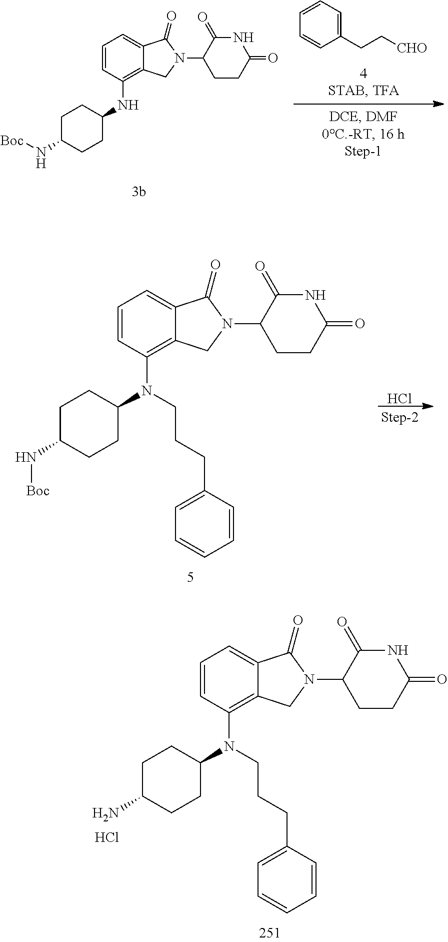

### Step-1: tert-butyl ((1r,4r)-4-((2-(2,6-dioxopiperidin-3-yl)-1-oxoisoindolin-4-yl) (3-phenylpropyl) amino) cyclohexyl) carbamate

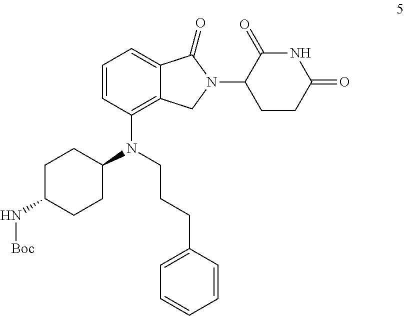 [SMILES: CC(C)(C)OC(=O)N[C@H]1CC[C@H](N(CCCc2ccccc2)c2cccc3c2CN(C2CCC(=O)NC2=O)C3=O)CC1]

To a stirred solution of tert-butyl ((1r,4r)-4-((2-(2,6-dioxopiperidin-3-yl)-1-oxoisoindolin-4-yl) amino) cyclohexyl) carbamate 3b (0.15 g, 0.329 mmol, 1 equiv) and 3-phenylpropanal 4 (0.529 g, 3.94 mmol, 12 equiv) in DCE (3 mL), DMF (1.5 mL) were added TFA (0.051 mL, 0.657 mmol, 2 equiv) and sodium triacetoxyborohydride (0.279 g, 1.314 mmol, 4 equiv) at 0° C. The resulting reaction mixture was stirred at room temperature overnight. Upon completion of the reaction (as confirmed by LCMS analysis) the reaction mixture was poured into ice cold water (10 mL) and extracted with EtOAc (3×25 mL). The combined organic layer was washed with brine (15 mL), dried over anhydrous sodium sulphate, filtered and concentrated to get the crude product. The crude compound was purified by preparative HPLC (Column: XSelect C18-250, Mobile phase: 0.1% FA in Water/ACN, Flow-rate: 15 mL/min), pure fractions were collected and lyophilized to get the desired product tert-butyl ((1r,4r)-4-((2-(2,6-dioxopiperidin-3-yl)-1-oxoisoindolin-4-yl) (3-phenylpropyl) amino) cyclohexyl) carbamate 5 (0.06 g, 0.103 mmol, 31.3% yield) as off-white solid.

¹H-NMR (400 MHz, DMSO-d6): 10.97 (s, 1H), 7.41 (t, J=8.00 Hz, 1H), 7.32-7.22 (m, 5H), 7.19-7.09 (m, 2H), 6.68 (d, J=7.60 Hz, 1H), 5.10 (q, J=4.80 Hz, 1H), 4.33 (d, J=17.20 Hz, 1H), 4.24 (d, J=17.20 Hz, 1H), 3.15-3.12 (m, 3H), 2.99-2.86 (m, 2H), 2.68-2.51 (m, 4H), 2.02-1.99 (m, 1H), 1.80-1.72 (m, 4H), 1.54-1.47 (m, 4H), 1.37 (s, 9H), 1.23-1.16 (m, 2H).

LCMS: 575.4 (M+H). Method: Column: Atlantis dC18 (50×4.6) 5μ, Mobile phase A: 0.1% FA in H₂O, B: ACN, Flow Rate: 1.5 mL/min, Rt (min): 3.125, Area (%): 99.391.

HPLC: Method info A: 0.1% TFA in H₂O, B: ACN, Column: XBridge C8 (50 x4.6) mm, 3.5 μm; Flow Rate: 2.0 mL/min; Rt (min): 4.460; Area (%): 98.345.

### Step-2: 3-(4-(((1r,4r)-4-aminocyclohexyl) (3-phenylpropyl) amino)-1-oxoisoindolin-2-yl) piperidine-2,6-dione hydrochloride

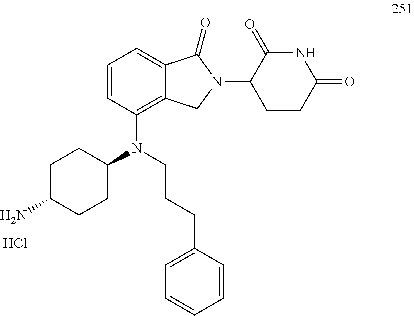 [SMILES: Cl ; N[C@H]1CC[C@H](N(CCCc2ccccc2)c2cccc3c2CN(C2CCC(=O)NC2=O)C3=O)CC1]

To a stirred solution of tert-butyl ((1r,4r)-4-((2-(2,6-dioxopiperidin-3-yl)-1-oxoisoindolin-4-yl) (3-phenylpropyl) amino) cyclohexyl) carbamate 5 (0.06 g, 0.103 mmol, 1 equiv) in DCM (2 mL) was added 4N HCl in EtOAc (0.5 mL, 2 mmol, 19.24 equiv) at 0° C. The resultant reaction mixture was allowed to stir at room temperature for 2.5 h. Upon completion of the reaction (as confirmed by TLC analysis, 100% EtOAc, Rf˜0.1), the reaction mixture was concentrated under reduced pressure to get the crude product. The crude compound was washed by EtOAc (2 mL), dried under vacuum, dissolved in 3 mL of ACN-water mixture (1:1) and lyophilized to get the desired product 3-(4-(((1r,4r)-4-aminocyclohexyl) (3-phenylpropyl) amino)-1-oxoisoindolin-2-yl) piperidine-2,6-dione hydrochloride 251 (0.052 g, 0.100 mmol, 97.08% yield) as off-white solid.

¹H-NMR (400 MHz, DMSO-d6): 10.98 (s, 1H), 7.87 (br s, 3H), 7.43-7.22 (m, 5H), 7.16-7.09 (m, 3H), 5.12 (q, J=5.20 Hz, 1H), 4.32-4.23 (m, 2H), 3.15-2.97 (m, 2H), 2.96-2.88 (m, 3H), 2.63-2.54 (m, 2H), 2.02-1.96 (m, 3H), 1.94-1.79 (m, 2H), 1.65-1.45 (m, 4H), 1.39-1.34 (m, 2H).

LCMS: 475.4 (M+H). Method: Column: Atlantis dC18 (50×4.6) 5 μm, Mobile phase A: 0.1% FA in H₂O, B: ACN, Flow Rate: 1.5 mL/min, Rt (min): 2.231, Area (%): 99.161.

HPLC: Method info A: 0.1% TFA in H₂O, B: ACN, Column: XBridge C8 (50 x4.6) mm, 3.5 μm; Flow Rate: 2.0 mL/min; Rt (min): 3.1; Area (%): 98.568.

### Hydrochloride Salt Form of Compound 255

### 3-(4-((2-(1-(aminomethyl) cyclohexyl) ethyl) (pentyl)amino)-1-oxoisoindolin-2-yl) piperidine-2,6-dione, Formic Acid

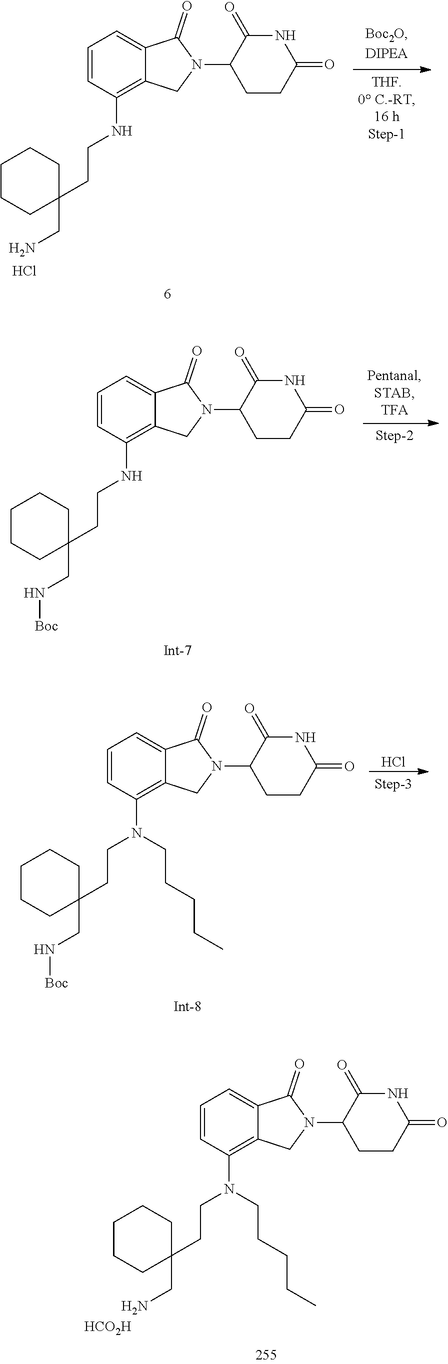

### Step-1: tert-butyl ((1-(2-((2-(2,6-dioxopiperidin-3-yl)-1-oxoisoindolin-4-yl) amino) ethyl) cyclohexyl) methyl) carbamate

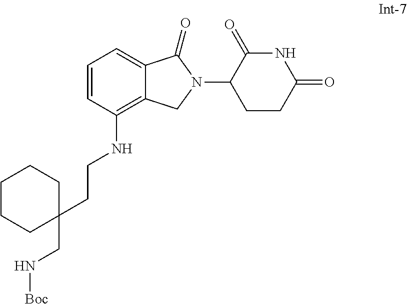 [SMILES: CC(C)(C)OC(=O)NCC1(CCNc2cccc3c2CN(C2CCC(=O)NC2=O)C3=O)CCCCC1]

To a stirred solution of 3-(4-((2-(1-(aminomethyl) cyclohexyl) ethyl) amino)-1-oxoisoindolin-2-yl) piperidine-2,6-dione hydrochloride 6 (0.1 g, 0.230 mmol, 1.0 equiv) in THF (5 mL) was added DIPEA (0.06 mL, 0.230 mmol, 1.5 equiv) followed by Boc-anhydride (0.08 mL, 0.230 mmol, 1.5 equiv) at 0° C. and the reaction mixture was allowed to warm and stirred at room temperature for overnight. Upon completion of reaction (as confirmed by UPLC), the reaction mixture was diluted with water (10 mL) and extracted with EtOAc (3×15 mL). The combined organic layer was washed with brine (20 mL), dried over anhydrous sodium sulphate, filtered and distilled under reduced pressure to afford the crude product tert-butyl ((1-(2-((2-(2,6-dioxopiperidin-3-yl)-1-oxoisoindolin-4-yl) amino) ethyl) cyclohexyl) methyl) carbamate Int-7 (0.24 g) as pale-brown gum which was taken as such for the next step without further purification.

LCMS: 443.6 (M+H-Boc). Method: Column: XBridge C8 (50×4.6 mm) 3.5 μm, Mobile phase A: 0.1% TFA in water, Mobile phase B: 0.1% TFA in ACN, Flow Rate: 1.5 mL/min. Rt (min): 2.270, Area (%): 88.98.

### Step-2: tert-butyl ((1-(2-((2-(2,6-dioxopiperidin-3-yl)-1-oxoisoindolin-4-yl)(pentyl)amino)ethyl)cyclohexyl)methyl)carbamate

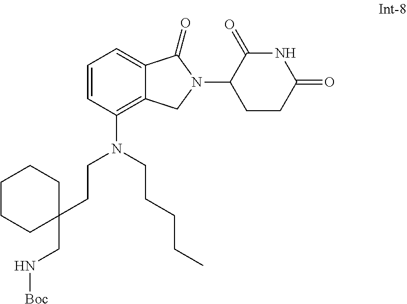 [SMILES: CCCCCN(CCC1(CNC(=O)OC(C)(C)C)CCCCC1)c1cccc2c1CN(C1CCC(=O)NC1=O)C2=O]

A mixture of tert-butyl ((1-(2-((2-(2,6-dioxopiperidin-3-yl)-1-oxoisoindolin-4-yl)amino)ethyl)cyclohexyl)methyl)carbamate Int-7 (0.24 g, 0.433 mmol, 1 equiv) and pentanal (0.460 mL, 4.332 mmol, 6 equiv) were dissolved in a mixture of DCE (2 mL) and DMF (1 mL) under nitrogen atmosphere, TFA (0.064 mL, 0.824 mmol, 1.9 equiv) and sodium triacetoxyborohydride (368 mg, 1.732 mmol, 6 equiv) were added in portions at 0° C. and stirred for 16 h at room temperature. Upon completion of the reaction (as confirmed by LCMS), the reaction mixture was diluted with DCM (20 mL), washed with water (3×10 mL), brine (20 mL), dried over anhydrous sodium sulphate, filtered and concentrated under reduced pressure to afford the crude product which was purified by preparative-HPLC (Column: XSelect C18-19.1×250 mm, Mobile phase: 0.1% FA in Water/ACN, Flow-rate: 15 mL/min) to afford the desired product tert-butyl ((1-(2-((2-(2,6-dioxopiperidin-3-yl)-1-oxoisoindolin-4-yl)(pentyl)amino)ethyl)cyclohexyl) methyl)carbamate Int-8 (0.079 g, 0.137 mmol, 8.95% yield) as off-white solid.

LCMS: 569.35 (M+H). Method: Column: Atlantis dC18 (50×4.6) nm, 5 μm, Mobile phase A: 0.1% FA in water, Mobile phase B: ACN, Flow Rate: 1.5 mL/min. Rt (min): 2.904, Area (%): 98.26.

HPLC: Method: Mobile phase A: 0.1% TFA in water, Mobile phase B: ACN, Column:X-Bridge C8(50×4.6) mm, 3.5 m, Flow Rate: 2.0 mL/min, Rt (min): 4.644, Area (%): 98.564.

### Step-3:3-(4-((2-(1-(aminomethyl)cyclohexyl)ethyl)(pentyl)amino)-1-oxoisoindolin-2-yl)piperidine-2,6-dione, Formic Acid

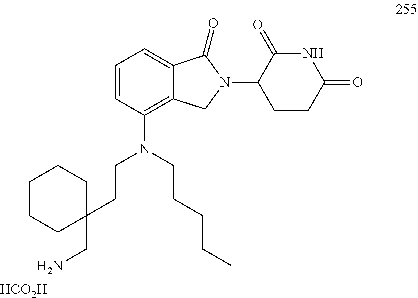 [SMILES: CCCCCN(CCC1(CN)CCCCC1)c1cccc2c1CN(C1CCC(=O)NC1=O)C2=O ; "HCO2H"]

To an ice cold solution of tert-butyl ((1-(2-((2-(2,6-dioxopiperidin-3-yl)-1-oxoisoindolin-4-yl)(pentyl)amino)ethyl)cyclohexyl)methyl)carbamate Int-8 (79 mg, 0.137 mmol, 1 equiv) in DCM (3 mL) was added HCl (4 M in 1,4-dioxane) (3.5 mL) at 0° C. and the reaction was stirred for 3 h at room temperature. Upon completion of reaction (as confirmed by LCMS), the reaction mixture was concentrated under reduced pressure and the solid was purified by prep-HPLC (Column: X-Bridge-C18, 19.1×250, Mobile phase: 0.1% FA in Water/ACN, Flow-rate: 15 mL/min) to afford 3-(4-((2-(1-(aminomethyl)cyclohexyl)ethyl)(pentyl)amino)-1-oxoisoindolin-2-yl)piperidine-2,6-dione, Formic Acid 255 (0.0075 g, 0.014 mmol, 10.22% yield) as off-white solid.

¹H-NMR (400 MHz, DMSO-d₆): δ 8.29 (s, 1H), 7.39 (t, J=7.60 Hz, 1H), 7.20 (d, J=6.80 Hz, 1H), 7.11 (d, J=8.00 Hz, 1H), 5.12 (q, J=5.20 Hz, 1H), 4.43 (d, J=17.20 Hz, 1H), 4.31 (d, J=16.80 Hz, 1H), 3.42-3.33 (m, 4H), 3.19-3.16 (m, 3H), 2.59 (s, 2H), 2.53-2.51 (m, 2H), 2.10-1.95 (m, 1H), 1.50-0.42 (m, 6H), 1.37-1.24 (m, 12H), 0.84 (t, J=6.80 Hz, 3H).

LCMS: 469.4 (M+H), Method: Mobile phase A: 0.1% FA in water, Mobile phase B: ACN, Column: Atlantis dC18 (50×4.6 mm) 5 μm, Flow Rate: 1.5 mL/min, Rt (min): 2.482, Area (%): 99.61.

HPLC: Method: Mobile phase A: 0.1% TFA in water, Mobile phase B: ACN, Column: X-Bridge C8(50×4.6) mm, 3.5 μm Flow Rate: 2.0 mL/min, Rt (min): 3.163, Area (%): 99.262.

### Hydrochloride Salt Form of Compound 267

### 3-(4-(((1R,4R)-4-aminocyclohexyl)(3,3-dimethylbutyl)amino)-1-oxoisoindolin-2-yl)piperidine-2,6-dione hydrochloride

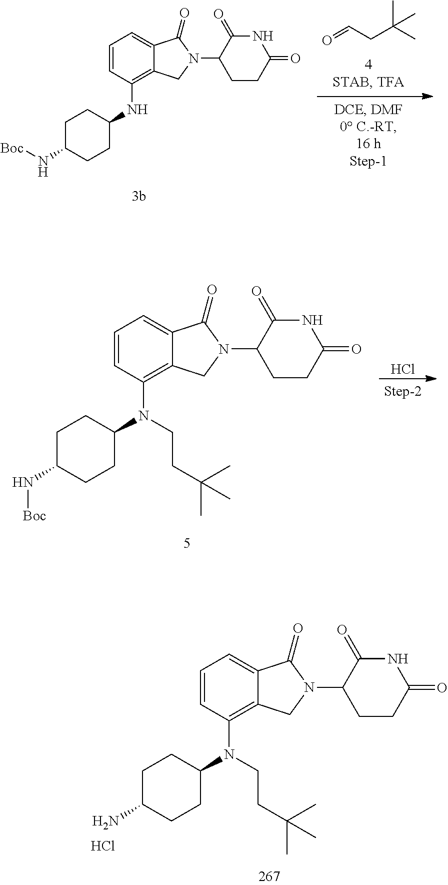

### Step-1: tert-butyl ((1R,4R)-4-((3,3-dimethylbutyl)(2-(2,6-dioxopiperidin-3-yl)-1-oxoisoindolin-4-yl)amino)cyclohexyl)carbamate

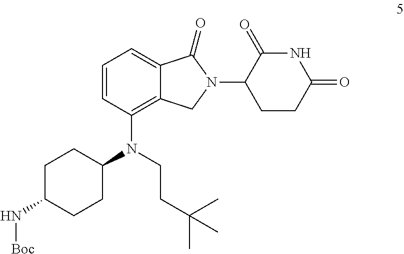 [SMILES: CC(C)(C)CCN(c1cccc2c1CN(C1CCC(=O)NC1=O)C2=O)[C@H]1CC[C@H](NC(=O)OC(C)(C)C)CC1]

A mixture of tert-butyl ((1R,4R)-4-((2-(2,6-dioxopiperidin-3-yl)-1-oxoisoindolin-4-yl)amino)cyclohexyl)carbamate 3b (0.150 g, 0.329 mmol, 1 equiv) and 3,3-dimethylbutanal 4 (0.33 g, 3.286 mmol, 10 equiv) were dissolved in a mixture of DCE (2 mL) and DMF (1 mL) under nitrogen atmosphere, TFA (0.076 mL, 0.986 mmol, 3 equiv) and sodium triacetoxyborohydride (0.418 g, 1.972 mmol, 6 equiv) were added in portions at 0° C. and stirred for 16 h at room temperature. Upon completion of the reaction (as confirmed by LCMS), the reaction mixture was diluted with DCM (20 mL), washed with water (3×10 mL), brine (20 mL), dried over anhydrous sodium sulphate, filtered and concentrated under reduced pressure to afford the crude product which was purified by preparative-HPLC (Column: XSelect C18-19.1×250 mm, Mobile phase: 0.1% FA in Water/ACN, Flow-rate: 15 mL/min) to afford the desired product tert-butyl ((1R,4R)-4-((3,3-dimethylbutyl)(2-(2,6-dioxopiperidin-3-yl)-1-oxoisoindolin-4-yl)amino)cyclohexyl)carbamate 5 (0.1 g, 0.184 mmol, 55.19% yield) as white solid.

LCMS: 541.3 (M+H). Method: Column: Atlantis dC18 (50×4.6) nm, 5 μm, Mobile phase A: 0.1% FA in water, Mobile phase B: ACN, Flow Rate: 1.5 mL/min. Rt (min): 2.625, Area (%): 98.68.

¹H-NMR (400 MHz, DMSO-d6): δ 10.98 (s, 1H), 7.42 (t, J=7.60 Hz, 1H), 7.30 (d, J=7.20 Hz, 1H), 7.23 (d, J=8.00 Hz, 1H), 6.69 (d, J=7.60 Hz, 1H), 5.11 (dd, J=5.20, 13.00 Hz, 1H), 4.27 (q, J=17.60 Hz, 2H), 3.33-3.11 (m, 3H), 3.01-2.86 (m, 2H), 2.68-2.61 (m, 2H), 2.02-2.01 (m, 1H), 2.00-1.81 (m, 4H), 1.55-1.52 (m, 2H), 1.37 (s, 9H), 1.23-1.12 (m, 4H), 0.86 (s, 9H).

### Step-2: 3-(4-(((1R,4R)-4-aminocyclohexyl)(3,3-dimethylbutyl)amino)-1-oxoisoindolin-2-yl)piperidine-2,6-dione hydrochloride

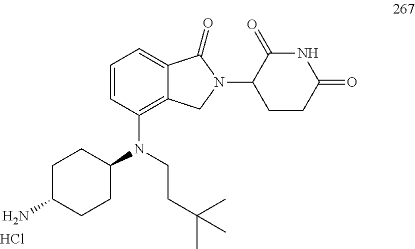 [SMILES: Cl ; CC(C)(C)CCN(c1cccc2c1CN(C1CCC(=O)NC1=O)C2=O)[C@H]1CC[C@H](N)CC1]

To an ice cold solution of tert-butyl ((1R,4R)-4-((3,3-dimethylbutyl)(2-(2,6-dioxopiperidin-3-yl)-1-oxoisoindolin-4-yl)amino)cyclohexyl)carbamate 5 (0.1 g, 0.184 mmol, 1 equiv) in DCM (3 mL) was added HCl (4 M in 1,4-dioxane) (4 mL) at 0° C. and the reaction was stirred for 3 h at room temperature. Upon completion of reaction (as confirmed by LCMS), the reaction mixture was evaporated to complete dryness under reduced pressure, washed with MTBE (10 mL) and lyophilized to afford the desired product 3-(4-(((1R,4R)-4-aminocyclohexyl)(3,3-dimethylbutyl)amino)-1-oxoisoindolin-2-yl)piperidine-2,6-dione hydrochloride 267 (84.88 mg, 0.177 mmol, 96% yield) as off-white solid.

¹H-NMR (400 MHz, DMSO-d6): δ 11.00 (s, 1H), 7.96 (s, 3H), 7.45-7.26 (m, 3H), 5.13 (dd, J=4.40, 12.80 Hz, 1H), 4.31-4.25 (m, 2H), 3.13-2.88 (m, 5H), 2.6-2.5 (m, 2H), 2.08-1.99 (m, 2H), 1.80-1.76 (m, 2H), 1.61-1.59 (m, 2H), 1.41-1.32 (m, 2H), 1.20-1.10 (m, 2H), 0.86 (s, 9H).

LCMS: 441.2 (M+H), Method: Mobile phase A: 0.1% FA in water, Mobile phase B: ACN, Column: Atlantis dC18 (50×4.6 mm) 5 μm, Flow Rate: 1.5 mL/min, Rt (min): 1.727, Area (%): 97.03.

HPLC: Method: Mobile phase A: 0.1% TFA in water, Mobile phase B: ACN, Column: X-Bridge C8(50×4.6) mm, 3.5 μm Flow Rate: 2.0 mL/min, Rt (min): 2.591, Area (%): 99.58.

### Hydrochloride Salt Form of Compound 268

### 3-(4-(((1r,4r)-4-aminocyclohexyl)(4,4-dimethylpentyl)amino)-1-oxoisoindolin-2-yl)piperidine-2,6-dione, HCl

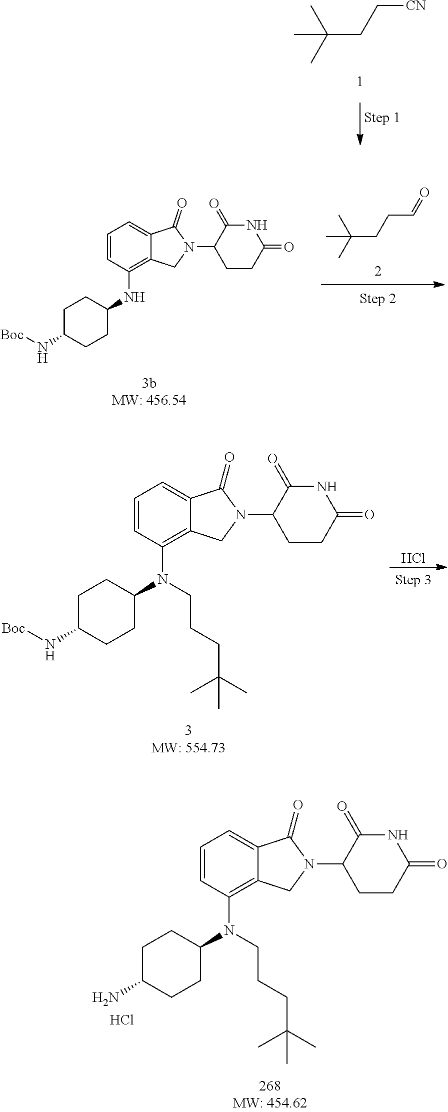

Step-1: 4,4-dimethylpentanal

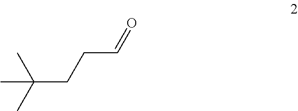

To a solution of 4,4-dimethylpentanenitrile 1 (1 g, 8.99 mmol, 1 equiv) in DCM (30 ml) was added DIBAL-H (1M soln., in toluene) (13.49 ml, 13.49 mmol, 1.5 equiv) at 0° C. and was allowed to stir at 0° C. for 1 h. Upon completion of reaction (as confirmed by TLC analysis, 50% EtOAc/pet ether, R_{f}˜0.6, KMnO₄), the reaction mixture was quenched with 3M HCl (10 ml) and filtered through Celite bed. The filtrate was washed with 1.5 N HCl (20 ml) and extracted with DCM (2×20 ml). The combined organic layer was dried over sodium sulphate and concentrated under vacuum to give the crude product 4,4-dimethylpentanal 2 (1 g, 8.76 mmol, 97% yield) as yellow liquid.

¹H-NMR (400 MHz, DMSO-d6): δ 9.80 (t, J=2.00 Hz, 1H), 2.44-2.37 (m, 2H), 1.55-1.52 (m, 2H), 0.95-0.92 (m, 9H).

### Step-2: tert-butyl ((1r,4r)-4-((4,4-dimethylpentyl)(2-(2,6-dioxopiperidin-3-yl)-1-oxoisoindolin-4-yl)amino)cyclohexyl)carbamate

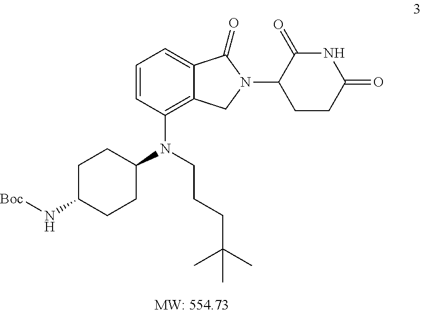 [SMILES: CC(C)(C)CCCN(c1cccc2c1CN(C1CCC(=O)NC1=O)C2=O)[C@H]1CC[C@H](NC(=O)OC(C)(C)C)CC1]

A mixture of tert-butyl ((1r,4r)-4-((2-(2,6-dioxopiperidin-3-yl)-1-oxoisoindolin-4-yl)amino)cyclohexyl)carbamate (150 mg, 0.329 mmol, 1 equiv) and 4,4-dimethylpentanal (901.6 mg, 7.896 mmol) in DCE (5 ml) and DMF (5.00 ml) was added TFA (168.6 mg, 1.479 mmol) and STAB (627 mg, 2.958 mmol, 9 equiv) in portions at 0° C. and stirred for 36 h at room temperature. Upon completion of the reaction (as confirmed by LCMS), the reaction mixture was diluted with DCM (20 mL), washed water (3×20 mL), brine (20 mL), dried over anhy. Na₂SO₄, filtered and concentrated to give the crude product. The crude product was purified by Preparative HPLC (Column: X-BRIDGE-C18, 0.1% HCOOH in water, flow rate: 20 mL/min). The pure fractions were concentrated under vacuum to give the desired product of tert-butyl((1r,4r)-4-((4,4-dimethyl pentyl) (2-(2,6-dioxopiperidin-3-yl)-1-oxoisoindolin-4-yl)amino)cyclohexyl)carbamate (40 mg, 0.072 mmol, 64.4% yield) as off white solid.

¹H-NMR (400 MHz, DMSO-d6): δ 10.98 (s, 1H), 7.42 (t, J=7.60 Hz, 1H), 7.27 (q, J=8.00 Hz, 2H), 6.70 (d, J=8.00 Hz, 1H), 5.12 (q, J=5.20 Hz, 1H), 4.29 (q, J=17.20 Hz, 2H), 3.34 (s, 1H), 3.06 (q, J=9.20 Hz, 2H), 3.00-2.88 (m, 2H), 2.57 (s, 2H), 1.99 (t, J=4.80 Hz, 1H), 1.77 (q, J=13.60 Hz, 4H), 1.53 (s, 2H), 1.37 (s, 9H), 1.24-1.12 (m, 6H), 0.83 (t, J=2.00 Hz, 2H), 0.73 (s, 9H). LCMS: 553.3 (M+H), Rt (min): 2.793, Area %: 99.919.

### Step-3: 3-(4-(((1r,4r)-4-aminocyclohexyl)(4,4-dimethylpentyl)amino)-1-oxoisoindolin-2-yl)piperidine-2,6-dione, HCl

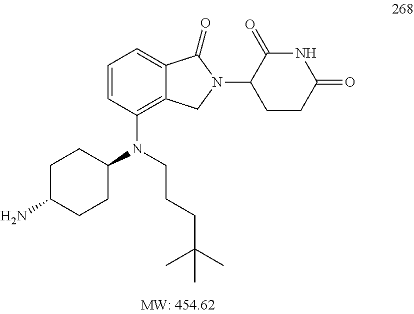 [SMILES: CC(C)(C)CCCN(c1cccc2c1CN(C1CCC(=O)NC1=O)C2=O)[C@H]1CC[C@H](N)CC1]

To an ice cold solution of tert-butyl ((1r,4r)-4-((4,4-dimethylpentyl)(2-(2,6-dioxopiperidin-3-yl)-1-oxoisoindolin-4-yl)amino)cyclohexyl)carbamate 3 (40 mg, 0.072 mmol, 1 equiv) in DCM (5 ml), HCl (4M in Ethyl acetate) (0.018 ml) was added and was stirred for 3 h at room temperature. Upon completion of reaction (as confirmed by LCMS), the reaction mixture was concentrated and the solid was washed with MTBE (10 ml), dried under vacuum and lyophilized to 3-(4-(((1r,4r)-4-aminocyclohexyl)(4,4-dimethylpentyl)amino)-1-oxoisoindolin-2-yl)piperidine-2,6-dione, HCl, 268 (36 mg, 0.071 mmol, 99% yield) as off white solid.

¹H-NMR (400 MHz, DMSO-d6): δ 10.99 (s, 1H), 7.87 (s, 3H), 7.44-7.29 (m, 3H), 5.14 (q, J=5.20 Hz, 1H), 4.29 (d, J=20.80 Hz, 2H), 3.08-2.92 (m, 5H), 2.01-1.92 (m, 3H), 1.78 (s, 2H), 1.53 (s, 2H), 1.35 (d, J=10.80 Hz, 3H), 1.19-1.11 (m, 4H), 0.74 (d, J=9.60 Hz, 8H).

LCMS: 455.3 (M+H), Method: Mobile phase: A: 0.1% FA in H₂O, Mobile phase: B: ACN, Column: Atlantis dC18 (50×4.6 mm) 5 μm, Flow Rate: 1.5 ml/min Rt (min): 1.860, Area %: 97.234.

HPLC: Method: Mobile phase: A: 0.1% TFA in water, Mobile phase: B: ACN, Column: X-Bridge C8(50×4.6) mm, 3.5 μm Flow: 2.0 mL/min, Rt (min): 3.027, Area %: 97.187.

### Hydrochloride Salt Form of Compound 273

### N-((1r,4r)-4-((4-aminobutyl)(2-(2,6-dioxopiperidin-3-yl)-1-oxoisoindolin-4-yl)amino)cyclohexyl)acetamide, 2HCl

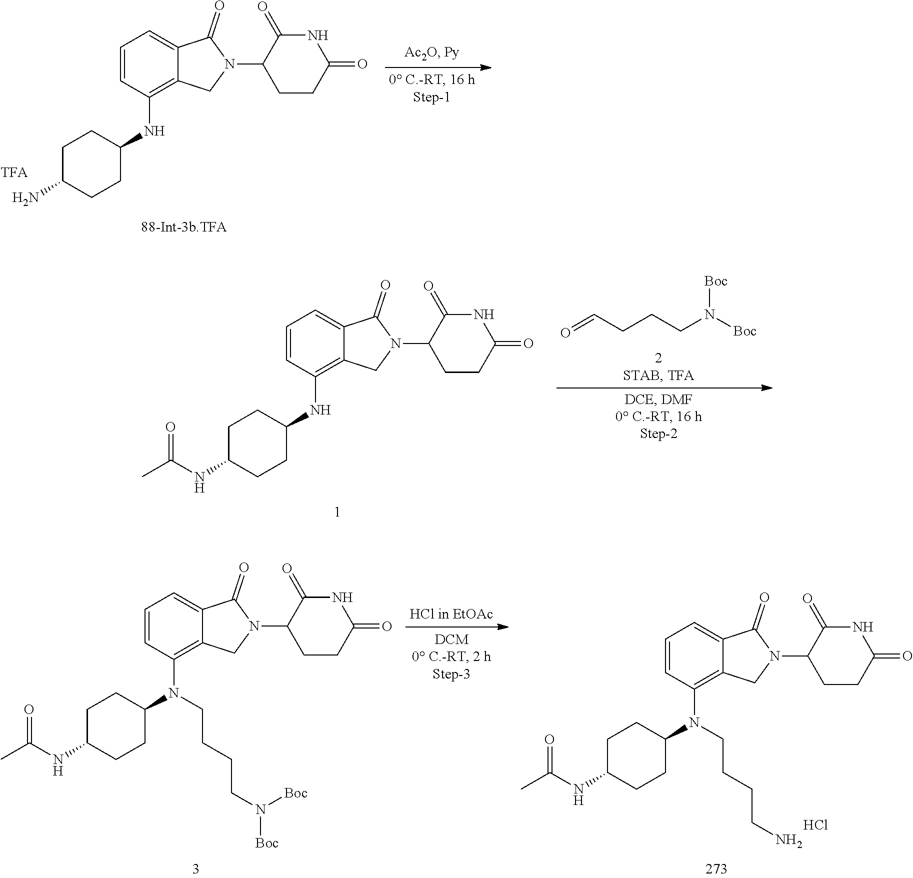

### Step-1: N-((1r,4r)-4-((2-(2,6-dioxopiperidin-3-yl)-1-oxoisoindolin-4-yl)amino)cyclohexyl)acetamide

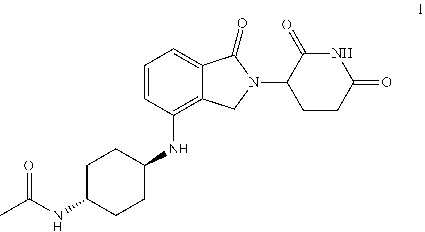 [SMILES: CC(=O)N[C@H]1CC[C@H](Nc2cccc3c2CN(C2CCC(=O)NC2=O)C3=O)CC1]

To a stirred solution of 3-(4-(((1r,4r)-4-aminocyclohexyl)amino)-1-oxoisoindolin-2-yl)piperidine-2,6-dione, 88-Int3b. TFA (0.15 g, 0.319 mmol, 1 equiv) in pyridine (3 mL) was added acetic anhydride (0.090 mL, 0.957 mmol, 3 equiv) at 0° C. under nitrogen atmosphere. The reaction mixture was then stirred overnight at room temperature. Upon completion (as confirmed by LCMS) the reaction mixture was diluted with water (20 mL) and extracted with EtOAc (3×40 mL). The combined organic layer was dried over anhydrous sodium sulphate, filtered and concentrated under reduced pressure to obtain crude product. The crude compound was purified by reverse-phase column chromatography (Grace® column: C18 40 μm, 50 g; flow rate: 15 mL/min; 0.1% aqueous FA/ACN mobile phase), and lyophilized to afford the desired product N-((1r,4r)-4-((2-(2,6-dioxopiperidin-3-yl)-1-oxoisoindolin-4-yl)amino)cyclohexyl)acetamide 1 (0.034 g, 0.085 mmol, 26.6% yield) to off-white solid.

¹H-NMR (400 MHz, DMSO-d₆): δ 11.02 (s, 1H), 7.78 (d, J=8.00 Hz, 1H), 7.28 (t, J=7.60 Hz, 1H), 6.92 (d, J=7.20 Hz, 1H), 6.82 (d, J=8.00 Hz, 1H), 5.31 (d, J=8.00 Hz, 1H), 5.13 (d, J=8.00 Hz, 1H), 4.22 (d, J=17.20 Hz, 1H), 4.12 (d, J=17.20 Hz, 1H), 3.34 (br s, 1H), 2.88-2.98 (m, 1H), 2.56-2.68 (m, 1H), 2.456-2.281 (m, 1H), 1.99-2.08 (m, 3H), 1.84-1.79 (m, 5H), 1.32-1.26 (m, 4H).

LCMS: 399.2 (M+H). Method: Mobile Phase A: 0.1% FA in water. Mobile Phase B: ACN. Flow Rate:1.5 mL/min. Column: Atlantis dC18 (50×4.6 mm) 5 μm. Rt (min): 1.607, Area (%): 99.514.

### Step-2: tert-butyl (4-(((1r,4r)-4-acetamidocyclohexyl)(2-(2,6-dioxopiperidin-3-yl)-1-oxoisoindolin-4-yl)amino)butyl)(tert-butoxycarbonyl)carbamate

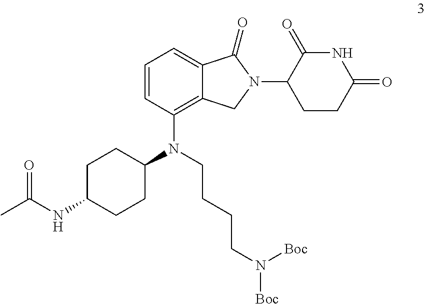 [SMILES: CC(=O)N[C@H]1CC[C@H](N(CCCCN(C(=O)OC(C)(C)C)C(=O)OC(C)(C)C)c2cccc3c2CN(C2CCC(=O)NC2=O)C3=O)CC1]

To a stirred solution of N-((1r,4r)-4-((2-(2,6-dioxopiperidin-3-yl)-1-oxoisoindolin-4-yl)amino)cyclohexyl)acetamide 1 (0.040 g, 0.100 mmol, 1 equiv) and tert-butyl (tert-butoxycarbonyl)(4-oxobutyl)carbamate 2 (0.058 g, 0.201 mmol, 2 equiv) in DCE (5 mL), DMF (1 mL), were added TFA (0.031 mL, 0.402 mmol, 4 equiv) and sodium triacetoxyborohydride (0.085 g, 0.402 mmol, 4 equiv) at 0° C. The resulting reaction mixture was stirred at room temperature for overnight. Upon completion (as confirmed by TLC, Mobile phase 10% MeOH in DCM, Rf˜0.2), the reaction mixture was poured into ice-cold water (10 mL) and extracted with DCM (3×20 mL). The combined organic layer was washed with brine (10 mL), dried over anhydrous sodium sulphate, filtered and concentrated to get the crude product. The crude compound was purified by preparative HPLC (Column: XSelect C18-250, Mobile phase: 0.1% FA in water/ACN, Flow-rate: 15 mL/min), and lyophilized to afford the desired product tert-butyl (4-(((1r,4r)-4-acetamidocyclohexyl)(2-(2,6-dioxopiperidin-3-yl)-1-oxoisoindolin-4-yl)amino)butyl)(tert-butoxycarbonyl)carbamate 3 (0.019 g, 0.028 mmol, 27.84% yield) as off-white solid.

¹H-NMR (400 MHz, DMSO-d6): δ 10.97 (s, 1H), 7.69 (d, J=8.00 Hz, 1H), 7.44-7.40 (m, 1H), 7.32-7.26 (m, 2H), 5.10 (d, J=4.80 Hz, 1H), 4.33 (d, J=17.60 Hz, 1H), 4.24 (d, J=17.20 Hz, 1H), 3.29-3.12 (m, 2H), 3.01-2.92 (m, 2H), 2.68-2.89 (m, 1H), 2.00-1.98 (m, 1H), 1.81-1.72 (m, 8H), 1.53-1.48 (m, 7H), 1.36 (d, J=12.40 Hz, 18H), 1.22-1.16 (m, 5H), LCMS: 670.3 (M+H). Method: Column: Atlantis dC18 (50×4.6) 5μ, Mobile phase A: 0.1% FA in water, B: ACN, Flow Rate: 1.5 mL/min, Rt (min): 2.323, Area (%): 99.045.

### Step-3: N-((1r,4r)-4-((4-aminobutyl)(2-(2,6-dioxopiperidin-3-yl)-1-oxoisoindolin-4-yl)amino)cyclohexyl)acetamide, HCl

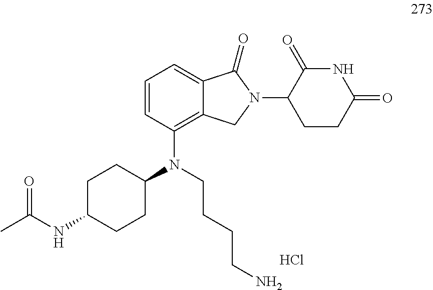 [SMILES: CC(=O)N[C@H]1CC[C@H](N(CCCCN)c2cccc3c2CN(C2CCC(=O)NC2=O)C3=O)CC1 ; Cl]

To a stirred solution of tert-butyl ((1r,4r)-4-((2-(1-methyl-2,6-dioxopiperidin-3-yl)-1-oxoisoindolin-4-yl)(pentyl)amino)cyclohexyl)carbamate 3 (0.019 g, 0.028 mmol, 1 equiv) in DCM (3 mL) was added 4N HCl in EtOAc (0.177 ml, 0.709 mmol, 25 equiv) at 0° C. The resultant reaction mixture was warmed to room temperature and stirred for 2 h. Upon completion of the reaction (as confirmed by TLC, 100% EtOAc, Rf˜0.1), the reaction mixture was concentrated under reduced pressure to get the crude as colourless gummy solid. The obtained solid was washed with MTBE (5 mL), dried under vacuum and lyophilized to afford the desired product N-((1r,4r)-4-((4-aminobutyl)(2-(2,6-dioxopiperidin-3-yl)-1-oxoisoindolin-4-yl)amino)cyclohexyl)acetamide, HCl 273 (0.0142 g, 0.026 mmol, 99.1% yield) as off-white solid.

¹H-NMR (400 MHz, DMSO-d₆): δ 10.98 (s, 1H), 7.72-7.70 (m, 3H), 7.43-7.31 (m, 3H), 5.12 (q, J=4.80 Hz, 1H), 4.36 (d, J=16.40 Hz, 1H), 4.24 (d, J=32.00 Hz, 1H), 3.48-3.48 (m, 2H), 3.14-3.04 (m, 3H), 2.88-2.94 (m, 1H), 2.72-2.67 (m, 1H), 2.68-2.59 (m, 2H), 2.05-2.01 (m, 1H), 1.75-1.82 (m, 7H), 1.49-1.59 (m, 4H), 1.25-1.29 (m, 5H).

LCMS: 470.4 (M-Boc), Method: Mobile phase A: 0.1% TFA in water, Mobile phase B: ACN, Column: XBridge C8 (50×4.6 mm) 3.5 μm, Flow Rate: 1.5 mL/min Rt (min): 0.879, Area (%): 98.645.

HPLC: Method: Mobile phase A: 0.1% FA in water, Mobile phase B: ACN, Column: X Bridge C8 (50×4.6) mm, 3.5 m, Flow Rate: 2.0 mL/min, Rt (min): 1.448, Area (%): 98.896.

### Compounds 305 and 307

### (1R,4R)-4-((2-(2,6-dioxopiperidin-3-yl)-1-oxoisoindolin-4-yl)(pentyl)amino)cyclohexane-1-carboxamide and (1S,4S)-4-((2-(2,6-dioxopiperidin-3-yl)-1-oxoisoindolin-4-yl)(pentyl)amino)cyclohexane-1-carboxamide

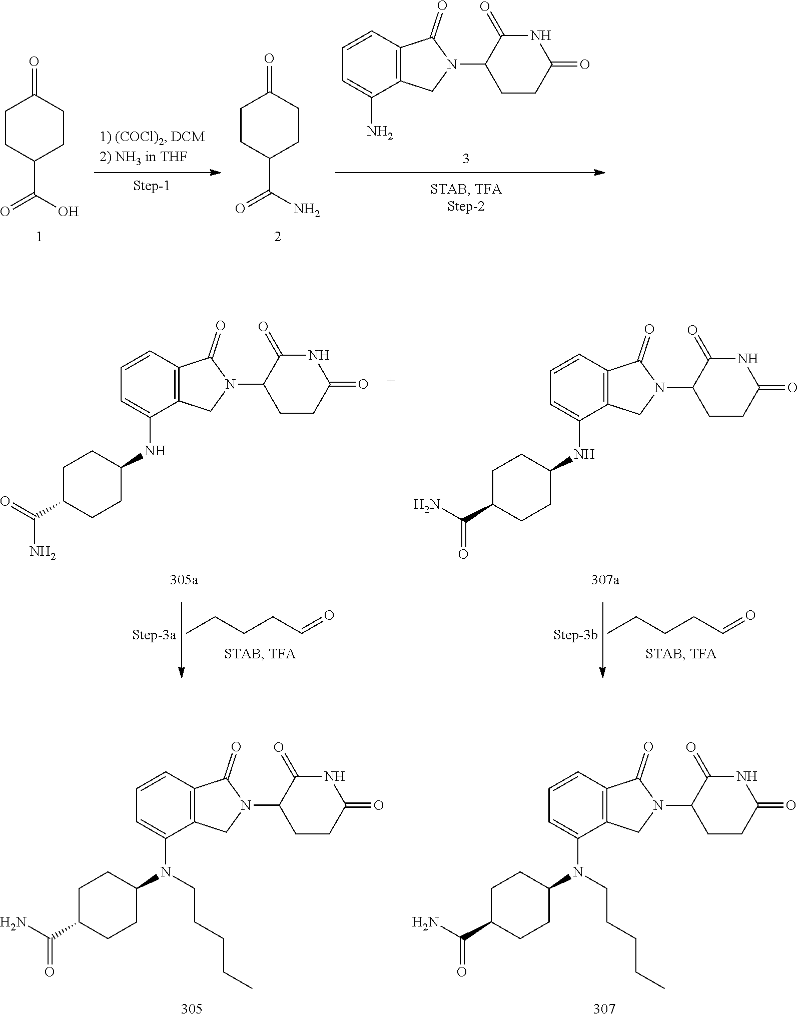

### Step-1: 4-oxocyclohexane-1-carboxamide

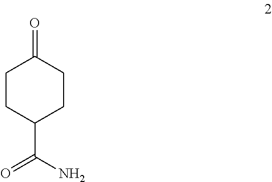

To an ice cold solution of 4-oxocyclohexane-1-carboxylic acid 1 (1 g, 7.03 mmol, 1 equiv) in DCM (18 mL) was added oxalyl chloride (1.231 ml, 14.07 mmol, 1.7 equiv), 0.1 mL DMF at 0° C. and stirred at room temperature for 2 h. Upon completion of the reaction (as confirmed by TLC analysis as methyl ester, 100% EtOAc, Rf˜0.7), the reaction mixture was concentrated under reduced pressure and dried completely under reduced pressure.

The resulted acid chloride mixture was dissolved in THF (1.5 mL) and added dropwise to a pre-cooled solution of NH₃ (0.5M soln. in THF, 34 mL) at −78° C. The reaction mixture was warmed to room temperature and stirred for overnight. Upon completion of the reaction (as confirmed by TLC analysis, 60% EtOAc in pet ether, Rf˜0.2), the reaction mixture was concentrated and resulted residue was poured into ice cold solution of sat. NaHCO₃ solution (100 mL) and extracted with EtOAc (2×75 mL). The combined organic layer was washed with brine (100 mL), dried over Na₂SO₄, filtered and concentrated to afford 4-oxocyclohexane-1-carboxamide 2 (679 mg, 4.56 mmol, 64.9% yield) as pale yellow solid.

LCMS: 142.1 (M+H). Method: Atlantis dC18 (50*4.6) 5 μm, Mobile phase A: 0.1% FA in H₂O, Mobile phase B: ACN, Flow Rate:1.5 mL/min. Rt (min): 0.841, Area (%): 94.890.

¹H NMR (400 MHz, DMSO-d6): δ 7.36 (s, 1H), 6.86 (s, 1H), 2.56-2.53 (m, 1H), 2.38-2.25 (m, 2H), 2.24-2.19 (m, 2H), 2.03-2.00 (m, 2H), 1.80-1.73 (m, 2H).

### Step-2: (1R,4R)-4-((2-(2,6-dioxopiperidin-3-yl)-1-oxoisoindolin-4-yl)amino)cyclohexane-1-carboxamide and (1S,4S)-4-((2-(2,6-dioxopiperidin-3-yl)-1-oxoisoindolin-4-yl)amino)cyclohexane-1-carboxamide

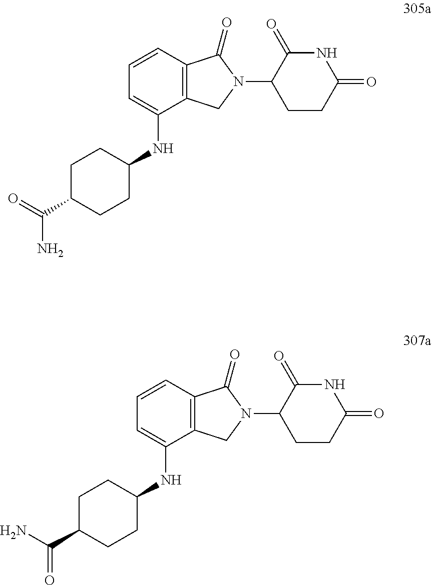 [SMILES: NC(=O)[C@H]1CC[C@H](Nc2cccc3c2CN(C2CCC(=O)NC2=O)C3=O)CC1 ; NC(=O)[C@H]1CC[C@@H](Nc2cccc3c2CN(C2CCC(=O)NC2=O)C3=O)CC1]

A mixture of 3-(4-amino-1-oxoisoindolin-2-yl)piperidine-2,6-dione 3 (150 mg, 0.579 mmol, 1 equiv) and 4-oxocyclohexane-1-carboxamide 2 (679 mg, 4.56 mmol, 8.3 equiv) were dissolved in a mixture of DCE (3 mL) and DMF (2 mL) under nitrogen atmosphere, TFA (0.05 ml, 0.649 mmol, 1.1 equiv) and sodium triacetoxyborohydride (500 mg, 2.359 mmol, 4 equiv) were added in portions at 0° C. and stirred for 16 h at room temperature. Upon completion of the reaction (as confirmed by LCMS), the reaction mixture was diluted with DCM (10 mL), washed with water (3×5 mL), brine (10 mL), dried over anhydrous sodium sulphate, filtered and concentrated under reduced pressure to afford the crude product which was purified by preparative-HPLC method (Method info: Column: XBridge-C18 19.1×250 mm, Mobile phase combination: 0.1% FA/ACN, Flow rate: 15 mL/min) to separate Peak I as trans isomer (1R,4R)-4-((2-(2,6-dioxopiperidin-3-yl)-1-oxoisoindolin-4-yl)amino)cyclohexane-1-carboxamide 305a (0.075 g, 0.183 mmol, 18.36% yield) and Peak II as cis isomer (1S,4S)-4-((2-(2,6-dioxopiperidin-3-yl)-1-oxoisoindolin-4-yl)amino)cyclohexane-1-carboxamide 307a (100 mg, 0.258 mmol, 3.96% yield) as off white solid.

Peak I (305a):

LCMS: 385.3 (M+H). Method: Atlantis dC18 (50*4.6) 5 μm, Mobile phase A: 0.1% FA in H₂O, Mobile phase B: ACN, Flow Rate:1.5 mL/min. Rt (min): 1.971, Area (%): 99.682.

¹H NMR (400 MHz, DMSO-d6): δ 8.15 (s, 1H), 7.29-7.21 (m, 2H), 6.91 (d, J=7.20 Hz, 1H), 6.81 (d, J=8.00 Hz, 1H), 6.68 (s, 1H), 5.29 (d, J=8.00 Hz, 1H), 5.13 (dd, J=4.40, 13.20 Hz, 1H), 4.17 (q, J=17.20 Hz, 2H), 2.98-2.89 (m, 1H), 2.65-2.60 (m, 1H), 2.37-2.24 (m, 1H), 2.12-2.02 (m, 5H), 1.82-782.00 (m, 2H), 1.51-1.45 (m, 2H), 1.33-1.23 (m, 2H).

Peak H (307a)

LCMS: 385.3 (M+H). Method: Atlantis dC18 (50*4.6) 5 μm, Mobile phase A: 0.1% FA in H₂O, Mobile phase B: ACN, Flow Rate:1.5 mL/min. Rt (min): 2.093, Area (%): 99.644.

¹H NMR (400 MHz, DMSO-d6): δ 11.01 (s, 1H), 7.29-7.18 (m, 2H), 6.92 (s, 1H), 6.79-6.72 (m, 2H), 5.23-5.22 (m, 1H), 5.15-5.10 (m, 1H), 4.22 (q, J=17.60 Hz, 2H), 3.51 (br s, 2H), 2.97-2.92 (m, 1H), 2.51-2.50 (m, 2H), 2.30-2.25 (m, 1H), 2.08-2.02 (m, 1H), 1.90-1.87 (m, 2H), 1.68-1.56 (m, 3H), 1.55-1.53 (m, 2H).

### Step-3a: (1R,4R)-4-((2-(2,6-dioxopiperidin-3-yl)-1-oxoisoindolin-4-yl)(pentyl)amino)cyclohexane-1-carboxamide

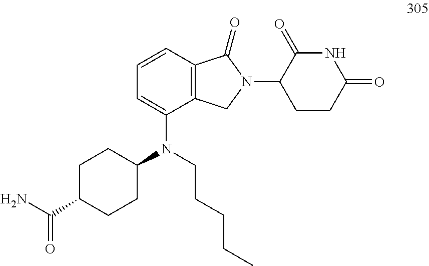 [SMILES: CCCCCN(c1cccc2c1CN(C1CCC(=O)NC1=O)C2=O)[C@H]1CC[C@H](C(N)=O)CC1]

To a mixture of (1r,4r)-4-((2-(2,6-dioxopiperidin-3-yl)-1-oxoisoindolin-4-yl)amino)cyclohexane-1-carboxamide 5 (76 mg, 0.198 mmol, 1 equiv) and pentanal (102.2 mg, 1.186 mmol, 6 equiv) in DCE (2 ml)/DMF (0.5 ml) was added TFA (90.2 mg, 0.79 mmol, 4 equiv) at 0° C. followed by STAB (336 mg, 1.582 mmol, 6 equiv) and the reaction was allowed to stir at room temperature for 24 h. Upon completion of reaction (as confirmed by LCMS), the reaction mixture was quenched with water (5 ml) and extracted with DCM (3×5 mL). The combined organic layer was washed with brine (10 mL), dried over anhydrous sodium sulphate, filtered and concentrated under vacuum to give the crude which was purified by preparative HPLC (Column: XBridge C18-19.1×150 mm; Method: 0.1% FA in ACN, flow rate: 20 ml/min) to get (1r,4r)-4-((2-(2,6-dioxopiperidin-3-yl)-1-oxoisoindolin-4-yl)(pentyl)amino)cyclohexane-1-carboxamide 305 (6.17 mg, 0.014 mmol, 8.81% yield) as white solid.

LCMS: 455.4 (M+H), Method: Mobile phase:A:0.1% FA in H₂O, Mobile phase:B: ACN, Column:Atlantis dC18 (50×4.6 mm) 5 μm, Flow Rate:1.5 ml/min Rt (min): 1.912, Area %: 98.205.

¹H-NMR (400 MHz, DMSO-d6): δ 10.98 (s, 1H), 7.41 (t, J=7.60 Hz, 1H), 7.27 (q, J=8.00 Hz, 2H), 7.16 (s, 1H), 6.67 (s, 1H), 5.09 (s, 1H), 4.30 (q, J=17.20 Hz, 2H), 3.16 (t, J=4.40 Hz, 3H), 2.68-2.67 (m, 1H), 2.67-2.61 (m, 1H), 2.00 (d, J=4.80 Hz, 2H), 1.78 (d, J=12.00 Hz, 4H), 1.42 (d, J=6.80 Hz, 4H), 1.22 (t, J=5.60 Hz, 7H), 0.80 (t, J=6.80 Hz, 3H).

HPLC: Method: Mobile phase:A:0.1% FA in water, Mobile phase: B: ACN, Column: X-Bridge C8(50×4.6) mm, 3.5 m Flow:2.0 mL/min, Rt (min): 2.787, Area %: 99.927.

### Step-3b: ((1S,4S)-4-((2-(2,6-dioxopiperidin-3-yl)-1-oxoisoindolin-4-yl)(pentyl)amino)cyclohexane-1-carboxamide

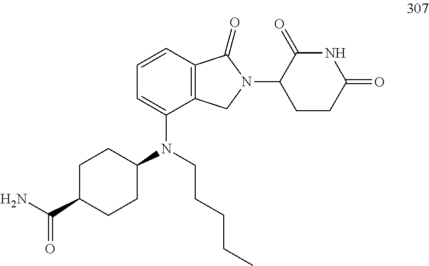 [SMILES: CCCCCN(c1cccc2c1CN(C1CCC(=O)NC1=O)C2=O)[C@H]1CC[C@@H](C(N)=O)CC1]

A mixture of (1S,4S)-4-((2-(2,6-dioxopiperidin-3-yl)-1-oxoisoindolin-4-yl)amino)cyclohexane-1-carboxamide 307a (100 mg, 0.258 mmol, 1 equiv). and pentanal (0.524 mL, 2.388 mmol, 9 equiv) were dissolved in a mixture of DCE (4 mL) and DMF (1 mL) under nitrogen atmosphere, TFA (0.082 ml, 1.062 mmol, 4 equiv) and sodium triacetoxyborohydride (505 mg, 2.388 mmol, 9 equiv) were added in portions at 0° C. and stirred for 16 h at room temperature. Upon completion of the reaction (as confirmed by LCMS), the reaction mixture was diluted with DCM (2×10 mL), washed with water (3×5 mL), brine (10 mL), dried over anhydrous sodium sulphate, filtered and concentrated under reduced pressure to afford the crude product which was purified by preparative-HPLC (Method info: Column: XBridge C18-19.1×150 mm; Method: 0.1% FA in ACN, flow rate: 15 ml/min) to afford (1S,4S)-4-((2-(2,6-dioxopiperidin-3-yl)-1-oxoisoindolin-4-yl)(pentyl)amino)cyclohexane-1-carboxamide 307 (18 mg, 0.039 mmol, 2.213% yield) as pale yellow solid.

LCMS: 455.4 (M+H), Method: Mobile phase:A:0.1% FA in H₂O, Mobile phase:B: ACN, Column: XBridge C8 (50×4.6 mm) 3.5 μm, Flow Rate:2 ml/min Rt (min): 1.115, Area %: 94.853.

¹H-NMR (400 MHz, DMSO-d6): δ 10.96 (s, 1H), 7.42 (t, J=7.60 Hz, 1H), 7.31-7.26 (m, 2H), 7.17 (s, 1H), 6.71 (s, 1H), 5.10 (d, J=5.20 Hz, 1H), 4.31 (q, J=16.80 Hz, 2H), 3.32-3.17 (m, 1H), 3.20-3.10 (m, 1H), 3.09-3.00 (m, 2H), 2.87-2.86 (m, 1H), 2.33-2.28 (m, 1H), 2.08-1.94 (m, 4H), 1.75-1.70 (m, 2H), 1.53-1.45 (m, 2H), 1.42-1.38 (m, 2H), 1.25-1.19 (m, 6H), 0.79 (t, J=6.80 Hz, 3H).

HPLC: Method: Mobile phase:A:0.1% TFA in water, Mobile phase: B: ACN, Column: X-Bridge C8(50×4.6) mm, 3.5 μm Flow:2.0 mL/min, Rt (min): 2.422, Area %: 98.379.

### Compounds 306 and 308

### (1R,4R)-4-((2-(2,6-dioxopiperidin-3-yl)-1-oxoisoindolin-4-yl)(pentyl)amino)-N-methylcyclohexane-1-carboxamide and (1S,4S)-4-((2-(2,6-dioxopiperidin-3-yl)-1-oxoisoindolin-4-yl)(pentyl)amino)-N-methylcyclohexane-1-carboxamide

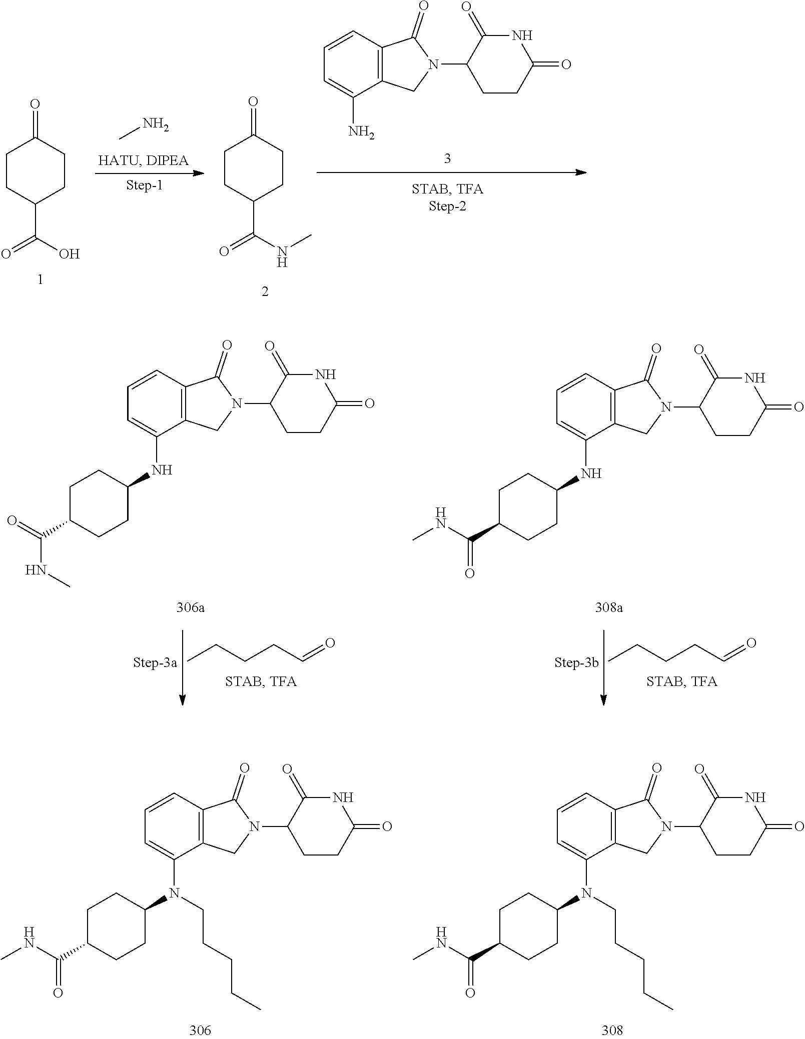

### Step-1: N-methyl-4-oxocyclohexane-1-carboxamide

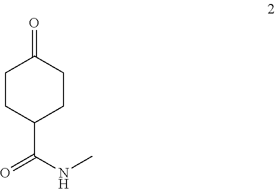

To a stirred solution of 4-oxocyclohexane-1-carboxylic acid 1 (1.5 g, 10.55 mmol, 1 equiv) in DMF (15 mL) were added DIPEA (5.51 ml, 31.7 mmol, 3 equiv) and HATU (6.02 g, 15.83 mmol, 1.5 equiv) at 0° C. methanamine (2M in THF) (5.28 ml, 10.55 mmol, 1 equiv) was then added at the same temperature and the reaction mixture was stirred at room temperature for 16 h. Upon completion of the reaction (as monitored by LCMS), the reaction mixture was diluted with EtOAc (2×50 mL), washed with water (3×50 mL), brine (100 mL), dried over anhydrous sodium sulphate, filtered and concentrated under reduced pressure to afford the crude product N-methyl-4-oxocyclohexane-1-carboxamide 2 (2 g, 12.28 mmol, 116% yield) as brown oil.

LCMS: Desired product did not ionize in LCMS. Method: Column: Atlantis dC18 (50*4.6) 55 μm, Mobile phase: A: 0.1% FA in H₂O B: ACN, Flow Rate: 1.5 ml/min. Rt (min): 1.060; Area %: 95.322.

### Step-2: (1R,4R)-4-((2-(2,6-dioxopiperidin-3-yl)-1-oxoisoindolin-4-yl)amino)-N-methylcyclohexane-1-carboxamide and (1S,4S)-4-((2-(2,6-dioxopiperidin-3-yl)-1-oxoisoindolin-4-yl)amino)-N-methylcyclohexane-1-carboxamide

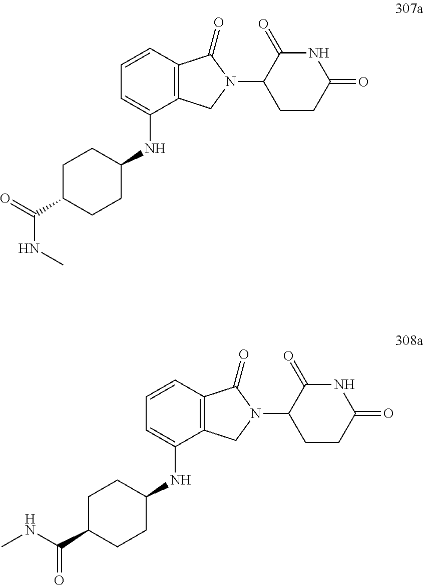 [SMILES: CNC(=O)[C@H]1CC[C@H](Nc2cccc3c2CN(C2CCC(=O)NC2=O)C3=O)CC1 ; CNC(=O)[C@H]1CC[C@@H](Nc2cccc3c2CN(C2CCC(=O)NC2=O)C3=O)CC1]

A mixture of 3-(4-amino-1-oxoisoindolin-2-yl)piperidine-2,6-dione (0.8 g, 3.09 mmol, 1 equiv) and N-methyl-4-oxocyclohexane-1-carboxamide (2.874 g, 18.52 mmol, 6 equiv) were dissolved in a mixture of DCE (4 mL) and DMF (4 mL) under nitrogen atmosphere, TFA (0.8 ml, 10.38 mmol, 3.3 equiv) and sodium triacetoxyborohydride (5.27 g, 24.87 mmol, 8 equiv) were added in portions at 0° C. and stirred for 16 h at room temperature. Upon completion of the reaction (as confirmed by LCMS), the reaction mixture was diluted with DCM (10 mL), washed with water (3×5 mL), brine (10 mL), dried over anhydrous sodium sulphate, filtered and concentrated under reduced pressure to afford the crude product which was purified by preparative-HPLC method (Method info: Column: Column: XSelect C18-250, Mobile phase combination: 10 mM ammonium bicarbonate in water/ACN, Flow rate: 15 mL/min) to separate Peak I as trans isomer (1R,4R)-4-((2-(2,6-dioxopiperidin-3-yl)-1-oxoisoindolin-4-yl)amino)-N-methylcyclohexane-1-carboxamide 306a (240 mg, 0.574 mmol, 7.62% yield) and Peak II as cis isomer (1S,4S)-4-((2-(2,6-dioxopiperidin-3-yl)-1-oxoisoindolin-4-yl)amino)-N-methylcyclohexane-1-carboxamide 308a (120 mg, 0.192 mmol, 2.56% yield) as off white solid.

Peak I (306a):

LCMS: 399.3 (M+H). Method: Atlantis dC18 (50*4.6) 5 μm, Mobile phase A: 0.1% FA in H₂O, Mobile phase B: ACN, Flow Rate:1.5 mL/min. Rt (min): 2.055, Area (%): 99.661.

¹H NMR (400 MHz, DMSO-d6): δ 11.02 (s, 1H), 7.68 (q, J=4.40 Hz, 1H), 7.27 (t, J=7.60 Hz, 1H), 6.87 (dd, J=8.00, 38.80 Hz, 2H), 5.29 (d, J=8.40 Hz, 1H), 5.13 (dd, J=5.20, 13.40 Hz, 1H), 3.00-2.85 (m, 1H), 2.31-2.27 (m, 1H), 2.08-2.02 (m, 4H), 1.78-1.74 (m, 2H), 1.54-1.50 (m, 2H), 1.22-1.20 (m, 2H)

Peak H (308a):

LCMS: 399.2 (M+H). Method: XBridge C8 (50×4.6 mm) 3.5 μm, Mobile phase A: 0.1% TFA in H₂O, Mobile phase B: 0.1% TFA in ACN, Flow Rate:1.5 mL/min. Rt (min): 1.315, Area (%): 63.938.

### Step-3a: (1R,4R)-4-((2-(2,6-dioxopiperidin-3-yl)-1-oxoisoindolin-4-yl)(pentyl)amino)-N-methylcyclohexane-1-carboxamide

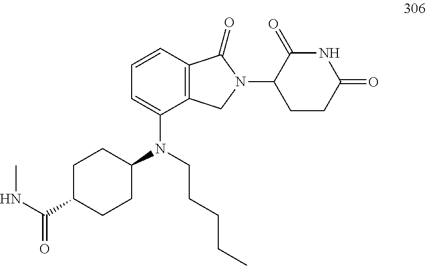 [SMILES: CCCCCN(c1cccc2c1CN(C1CCC(=O)NC1=O)C2=O)[C@H]1CC[C@H](C(=O)NC)CC1]

A mixture of ((1R,4R)-4-((2-(2,6-dioxopiperidin-3-yl)-1-oxoisoindolin-4-yl)amino)-N-methylcyclohexane-1-carboxamide 306a (240 mg, 0.574 mmol, 1 equiv) and pentanal (0.352 ml, 3.31 mmol, 5 equiv) were dissolved in a mixture of DCE (5 mL) and DMF (2 mL) under nitrogen atmosphere, TFA (0.05 ml, 0.663 mmol, 1 equiv) and sodium triacetoxyborohydride (421 mg, 1.988 mmol, 3 equiv) were added in portions at 0° C. and stirred for 16 h at room temperature. Upon completion of the reaction (as confirmed by LCMS), the reaction mixture was diluted with DCM (2×10 mL), washed with water (3×5 mL), brine (10 mL), dried over anhydrous sodium sulphate, filtered and concentrated under reduced pressure to afford the crude product which was purified by preparative HPLC (Method info: Instrument: S/DC/ARD/05-100, Column: XSELECT C-18 19.1-150; Method: 0.1% FA in ACN, flow rate: 15 ml/min) to afford (1R,4R)-4-((2-(2,6-dioxopiperidin-3-yl)-1-oxoisoindolin-4-yl)(pentyl)amino)-N-methylcyclohexane-1-carboxamide 306 (114.21 mg, 0.244 mmol, 6.34% yield) as yellow solid.

LCMS: 469.4 (M+H), Method: Mobile phase:A:0.1% FA in H₂O, Mobile phase:B: ACN, Column:Atlantis dC18 (50×4.6 mm) 5 μm, Flow Rate:1.5 ml/min Rt (min): 2.355, Area %: 99.202.

¹H-NMR (400 MHz, DMSO-d6): δ 7.47-7.45 (m, 2H), 7.37-7.35 (m, 1H), 5.15 (dd, J=5.20, 13.20 Hz, 1H), 4.45 (s, 2H), 3.19-3.15 (m, 3H), 2.93-2.82 (m, 2H), 2.71 (s, 3H), 2.61-2.56 (m, 1H), 2.23-2.21 (m, 1H), 2.15-2.05 (m, 1H), 1.93-1.87 (m, 4H), 1.57-1.50 (m, 4H), 1.35-1.27 (m, 6H), 0.86 (t, J=7.20 Hz, 3H).

HPLC: Method: Mobile phase:A:0.1% TFA in water, Mobile phase: B: ACN, Column: X-Bridge C8(50×4.6) mm, 3.5 μm Flow: 2.0 mL/min, Rt (min): 2.487, Area %: 99.947.

### Step-3b: (1S,4S)-4-((2-(2,6-dioxopiperidin-3-yl)-1-oxoisoindolin-4-yl)(pentyl)amino)-N-methylcyclohexane-1-carboxamide

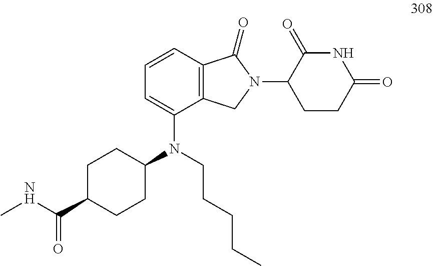 [SMILES: CCCCCN(c1cccc2c1CN(C1CCC(=O)NC1=O)C2=O)[C@H]1CC[C@@H](C(=O)NC)CC1]

A mixture of (1S,4S)-4-((2-(2,6-dioxopiperidin-3-yl)-1-oxoisoindolin-4-yl)amino)-N-methylcyclohexane-1-carboxamide 308a (120 mg, 0.192 mmol, 1 equiv). and pentanal (0.457 mL, 4.305 mmol, 12 equiv) were dissolved in a mixture of DCE (4 mL) and DMF (1 mL) under nitrogen atmosphere, TFA (0.2 mL, 2.060 mmol, 7.75 equiv) and sodium triacetoxyborohydride (1 g, 4.878 mmol, 14 equiv) were added in portions at 0° C. and stirred for 16 h at room temperature. Upon completion of the reaction (as confirmed by LCMS), the reaction mixture was diluted with DCM (2×10 mL), washed with water (3×5 mL), brine (10 mL), dried over anhydrous sodium sulphate, filtered and concentrated under reduced pressure to afford the crude product which was purified by preparative-HPLC (Method info: Column: XBridge C18-19.1×150 mm; Method: 0.1% FA in ACN, flow rate: 15 ml/min) to afford (1S,4S)-4-((2-(2,6-dioxopiperidin-3-yl)-1-oxoisoindolin-4-yl)(pentyl)amino)-N-methylcyclohexane-1-carboxamide 308 (8.74 mg, 0.019 mmol, 41.6% yield) as white solid.

LCMS: 469.4 (M+H), Method: Mobile phase:A:0.1% TFA in H2O, Mobile phase:B: ACN, Column: XBridge C8 (50×4.6 mm) 3.5 μm, Flow Rate:2 ml/min Rt (min): 1.161, Area %: 99.950.

¹H-NMR (400 MHz, DMSO-d6): 10.97 (s, 1H), 7.61-7.60 (m, 1H), 7.42 (t, J=7.60 Hz, 1H), 7.32-7.26 (m, 2H), 5.10 (dd, J=4.80, 13.00 Hz, 1H), 4.32 (q, J=17.20 Hz, 2H), 3.25-3.15 (m, 1H), 3.19-3.02 (m, 2H), 2.95-2.84 (m, 1H), 2.65-2.55 (m, 5H), 2.30-2.20 (m, 1H), 2.03-1.90 (m, 1H), 1.89-1.87 (m, 2H), 1.79-1.77 (m, 2H), 1.52-1.40 (m, 4H), 1.27-1.17 (m, 6H), 0.79 (t, J=7.20 Hz, 3H).

HPLC: Method: Mobile phase:A:0.1% TFA in water, Mobile phase: B: ACN, Column: X-Bridge C8(50×4.6) mm, 3.5 μm Flow:2.0 mL/min, Rt (min): 2.562, Area %: 99.915.

### Hydrochloride Salt Form of Compound 309

### 3-(4-(((1r,4r)-4-(ethylamino)cyclohexyl)(pentyl)amino)-1-oxoisoindolin-2-yl)piperidine-2,6-dione, HCl

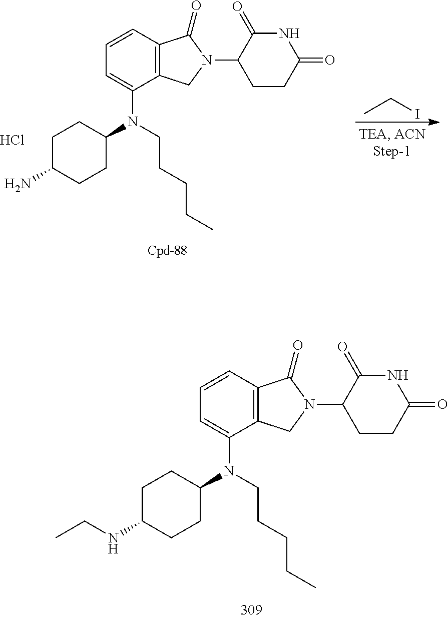

To a stirring solution of 3-(4-(((1r,4r)-4-aminocyclohexyl)(pentyl)amino)-1-oxoisoindolin-2-yl)piperidine-2,6-dione, HCl 88 (0.1 g, 0.216 mmol, 1 equiv) in ACN (1 mL) were added triethylamine (0.022 g, 0.216 mmol, 1 equiv) and 1-bromobutane (0.030 g, 0.194 mmol, 0.9 equiv) in portions at 0° C. and the reaction mixture was stirred at room temperature for 16 h. Upon completion of the reaction (as confirmed by LCMS), the reaction mixture was diluted with DCM (20 mL), washed with water (2×10 mL), brine (10 mL), dried over anhydrous sodium sulphate, filtered and concentrated to give the crude product. The crude product was purified by preparative HPLC (Column: YMC Triart C18; Mobile phase: 0.1% HCl in water, flow rate: 20 mL/min) to get the pure product 3-(4-(((1r,4r)-4-(ethylamino)cyclohexyl)(pentyl)amino)-1-oxoisoindolin-2-yl)piperidine-2,6-dione, HCl 309 (11.2 mg, 0.022 mmol, 10.12% yield) as off white solid.

¹H-NMR (400 MHz, DMSO-d6): δ 10.99 (s, 1H), 8.58 (s, 2H), 7.44 (s, 1H), 7.32 (d, J=13.20 Hz, 2H), 5.13 (q, J=4.80 Hz, 1H), 4.29 (q, J=16.00 Hz, 2H), 3.2-3.0 (m, 3H), 2.92 (q, J=7.60 Hz, 4H), 2.62-2.58 (m, 2H), 2.04 (q, J=5.20 Hz, 3H), 1.93-1.83 (m, 2H), 1.75-1.54 (m, 2H), 1.36 (d, J=12.40 Hz, 2H), 1.29-1.15 (m, 9H), 0.79 (t, J=6.80 Hz, 3H).

LCMS: 455.3 (M+H), Method: Mobile phase A: 0.1% TFA in water, Mobile phase B: ACN, Column: Atlantis dC18 (50×4.6 mm) 5 μm, Flow Rate: 1.5 mL/min, Rt (min): 1.328, Area (%): 99.590.

HPLC: Method: Mobile phase A: 0.1% TFA in water, Mobile phase B: ACN, Column: X-Bridge C8(50×4.6) mm, 3.5 μm, Flow Rate: 2.0 mL/min, Rt (min): 2.543, Area (%): 97.645.

### Hydrochloride Salt Form of Compound 310

### 3-(4-(((1r,4r)-4-(isopropylamino)cyclohexyl)(pentyl)amino)-1-oxoisoindolin-2-yl)piperidine-2,6-dione, HCl

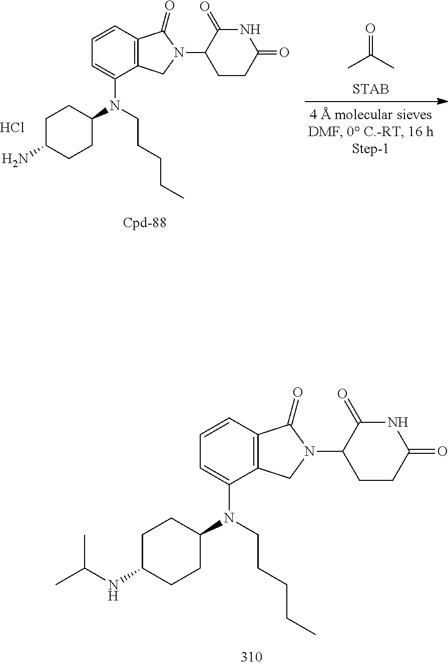

To a stirred mixture of 3-(4-(((1r,4r)-4-aminocyclohexyl)(pentyl)amino)-1-oxoisoindolin-2-yl)piperidine-2,6-dione.HCl 88 (0.05 g, 0.108 mmol, 1 equiv), acetone (0.627 g, 10.80 mmol, 100 equiv) and activated molecular sieves (size-4A, 300 mg) in DMF (1.5 mL), was added sodium triacetoxyborohydride (0.069 g, 0.324 mmol, 3 equiv) at 0° C. The reaction mixture was then warmed to room temperature and stirred for overnight. Upon completion of the reaction (as confirmed by LCMS), the reaction mixture was filtered and the filtrate was concentrated under vacuum to give the crude product which was purified by preparative-HPLC (Column: XSelect C18-19.1×250 mm, Mobile phase: 0.1% HCl in water, flow rate: 20 mL/min) to get the desired product 3-(4-(((1r,4r)-4-(isopropylamino)cyclohexyl)(pentyl)amino)-1-oxoisoindolin-2-yl)piperidine-2,6-dione.HCl 310 (11.2 mg, 0.022 mmol, 20.37% yield) as off white solid.

¹H-NMR (400 MHz, DMSO-d₆): δ 10.99 (s, 1H), 8.26 (s, 2H), 7.44 (t, J=7.60 Hz, 1H), 7.31 (q, J=7.60 Hz, 2H), 5.14 (q, J=5.20 Hz, 1H), 4.33 (d, J=17.20 Hz, 1H), 4.23 (d, J=17.20 Hz, 1H), 3.11-3.03 (m, 5H), 2.98-2.89 (m, 1H), 2.52-2.50 (m, 2H), 2.01 (t, J=10.40 Hz, 3H), 1.82 (d, J=11.20 Hz, 2H), 1.60 (q, J=3.60 Hz, 2H), 1.35 (q, J=13.60 Hz, 2H), 1.22 (t, J=6.40 Hz, 12H), 0.80 (t, J=6.80 Hz, 3H).

LCMS: 469.3 (M+H), Method: Mobile phase A: 0.1% TFA in water, Mobile phase B: ACN, Column: Atlantis dC18 (50×4.6 mm) 5 μm, Flow Rate: 1.5 mL/min, Rt (min): 1.898, Area (%): 97.636.

HPLC: Method: Mobile phase A: 0.1% TFA in water, Mobile phase B: ACN, Column: X-Bridge C8(50×4.6) mm, 3.5 μm, Flow Rate: 2.0 mL/min, Rt (min): 2.680, Area (%): 97.794.

### Hydrochloride Salt Form of Compound 311

### 3-(4-(((1r,4r)-4-(isobutylamino)cyclohexyl)(pentyl)amino)-1-oxoisoindolin-2-yl)piperidine-2,6-dione, HCl

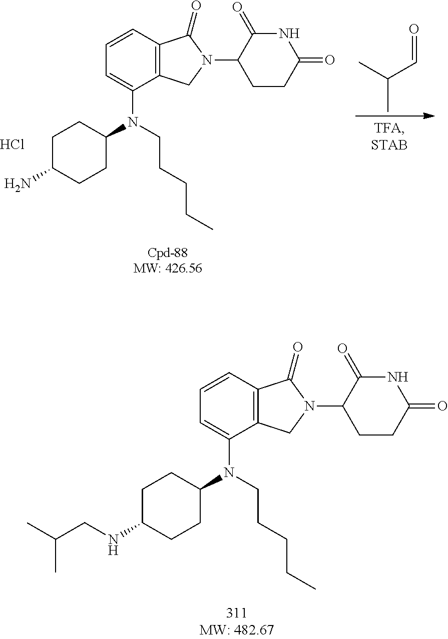

To a solution of 3-(4-(((1r,4r)-4-aminocyclohexyl)(pentyl)amino)-1-oxoisoindolin-2-yl)piperidine-2,6-dione, HCl 88 (60 mg, 0.130 mmol, 1 equiv) in a mixture of DCE (5 ml) and DMF (1 ml) was added activated molecular sieves (100 mg, 4 A° beads) and Isobutyraldehyde (9.34 mg, 0.130 mmol, 1 equiv) and stirred at room temperature for 8 h. Then STAB (41.2 mg, 0.194 mmol, 1.5 equiv) was added in portions at 0° C. and the reaction mixture was stirred at room temperature for 16 h. Upon completion of the reaction (as confirmed by LCMS), the reaction mixture was filtered and the filtrate was concentrated under vacuum to give the crude product which was purified by preparative HPLC (Column: YMC Triart C18, Mobile phase: 0.1% HCl in water) to get 3-(4-(((1r,4r)-4-(isobutylamino)cyclohexyl)(pentyl)amino)-1-oxoisoindolin-2-yl)piperidine-2,6-dione, HCl 311 (35 mg, 0.064 mmol, 31.0% yield) as pale yellow solid.

1H-NMR (400 MHz, DMSO-d6): δ 10.99 (s, 1H), 8.57 (s, 2H), 7.39 (m, 3H), 5.13 (q, J=5.20 Hz, 1H), 4.29 (d, J=19.60 Hz, 2H), 3.10 (s, 2H), 2.90-2.93 (m, 2H), 2.70 (q, J=3.60 Hz, 2H), 2.10 (d, J=8.80 Hz, 2H), 2.02-1.93 (m, 2H), 1.82 (s, 2H), 1.56-1.43 (m, 4H), 1.21 (s, 6H), 0.94 (d, J=6.80 Hz, 6H), 0.79 (d, J=6.00 Hz, 3H).

LCMS: 483.4 (M+H), Method: Mobile phase:A:0.1% TFA in H₂O, Mobile phase:B: ACN, Column: Atlantis dC18 (50×4.6 mm) 5 μm, Flow Rate:1.5 ml/min Rt (min): 2.484, Area %: 95.469.

HPLC: Method: Mobile phase:A:0.1% TFA in water, Mobile phase:B: ACN, Column:X-Bridge C8 (50×4.6) mm, 3.5 m; Flow: 2.0 mL/min, Rt (min): 2.876, Area %: 95.247.

### Hydrochloride Salt Form of Compound 312

### 3-(1-oxo-4-(pentyl((1r,4r)-4-(propylamino)cyclohexyl)amino)isoindolin-2-yl)piperidine-2,6-dione, HCl

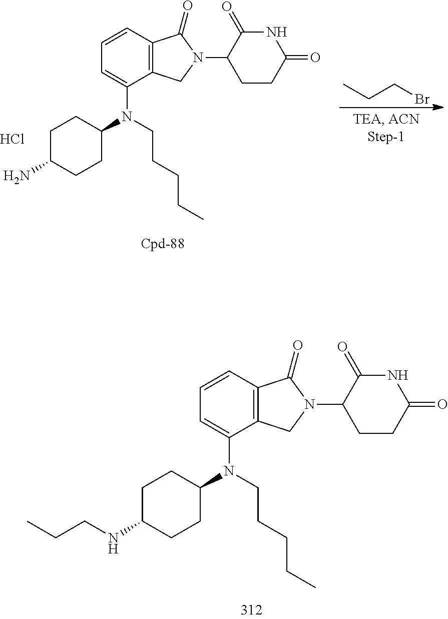

To a solution of 3-(4-(((1r,4r)-4-aminocyclohexyl)(pentyl)amino)-1-oxoisoindolin-2-yl)piperidine-2,6-dione.HCl 88 (0.1 g, 0.216 mmol, 1 equiv) in acetonitrile (1 mL) were added TEA (0.022 g, 0.216 mmol, 1 equiv) and 1-bromopropane (0.024 g, 0.194 mmol, 0.9 equiv) in portions at 0° C. and the reaction mixture was stirred at room temperature for 16 h. Upon completion of the reaction (as confirmed by LCMS), the reaction mixture was concentrated under reduced pressure to give the crude product, which was purified by preparative HPLC (Column: YMC Triart C18; Mobile phase: 0.1% HCl in water, flow rate: 20 mL/min) to get the desired product 3-(1-oxo-4-(pentyl((1r,4r)-4-(propylamino)cyclohexyl)amino)isoindolin-2-yl)piperidine-2,6-dione, HCl 312 (0.018 g, 0.035 mmol, 16.2% yield) as off white solid.

¹H-NMR (400 MHz, DMSO-d6): δ 11.00 (s, 1H), 8.71 (s, 2H), 7.40 (d, 3H), 5.13 (q, J=4.80 Hz, 1H), 4.30 (d, J=21.20 Hz, 2H), 3.11 (s, 2H), 2.97-2.93 (m, 2H), 2.90-2.81 (m, 2H), 2.53-2.51 (m, 2H), 2.07 (t, J=20.40 Hz, 4H), 1.83 (s, 2H), 1.65-1.57 (m, 4H), 1.40 (d, J=11.60 Hz, 2H), 1.20 (s, 6H), 0.91 (t, J=7.20 Hz, 3H), 0.79 (t, J=6.40 Hz, 3H).

LCMS: 469.3 (M+H), Method: Mobile phase A: 0.1% FA in water, Mobile phase B: ACN, Column: Atlantis dC18 (50×4.6 mm) 5 μm, Flow Rate: 1.5 mL/min Rt (min): 1.851, Area (%): 99.253.

HPLC: Method: Mobile phase A: 0.1% FA in water, Mobile phase B: ACN, Column: X-Bridge C8(50×4.6) mm, 3.5 m, Flow Rate: 2.0 mL/min, Rt (min): 2.878, Area (%): 98.154.

### Hydrochloride Salt Form of Compound 313

### 3-(4-(((1R,4R)-4-((cyclopropylmethyl)amino)cyclohexyl)(pentyl)amino)-1-oxoisoindolin-2-yl)piperidine-2,6-dione hydrochloride

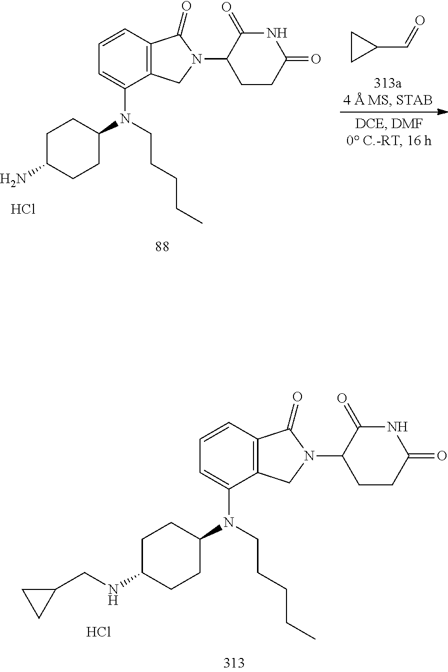

To a mixture of 3-(4-(((1R,4R)-4-aminocyclohexyl)(pentyl)amino)-1-oxoisoindolin-2-yl)piperidine-2,6-dione hydrochloride 88 (0.062 g, 0.134 mmol, 1 equiv), cyclopropanecarbaldehyde 313a (0.01 mL, 0.134 mmol, 1 equiv) and activated molecular sieves (4A, 300 mg) in a mixture of DCE (1 mL) and DMF (1 mL) was added sodium triacetoxyborohydride (0.028 g, 0.134 mmol, 1 equiv) at 0° C. The reaction mixture was warmed to room temperature and stirred overnight. Upon completion of the reaction (as confirmed by LCMS), the reaction mixture was diluted with DCM (15 mL), washed with water (3×10 mL), brine (15 mL), dried over anhydrous sodium sulphate, filtered and concentrated under reduced pressure to afford the crude product which was purified by preparative-HPLC (Column: XSelect C18-19.1×250 mm, Mobile phase: 0.1% HCl in Water/ACN, Flow-rate: 15 mL/min) to get 3-(4-(((1R,4R)-4-((cyclopropylmethyl)amino)cyclohexyl)(pentyl)amino)-1-oxoisoindolin-2-yl)piperidine-2,6-dione hydrochloride 313 (12.29 mg, 0.024 mmol, 2.66% yield) as white solid.

¹H NMR (400 MHz, DMSO-d6): δ 10.98 (s, 1H), 8.60 (s, 2H), 7.44-7.32 (m, 3H), 5.12 (dd, J=5.20, Hz, 1H), 4.45-4.15 (m, 2H), 2.00-2.85 (m, 5H), 2.70-2.60 (m, 2H), 2.51-2.50 (m, 2H), 2.10-2.07 (m, 4H), 2.90-2.75 (m, 2H), 1.65-1.50 (m, 2H), 1.45-1.30 (m, 2H), 1.21-1.20 (m, 6H), 1.10-1.00 (m, 1H), 0.80 (t, J=6.80 Hz, 3H), 0.59-0.55 (m, 2H), 0.35 (dd, J=1.20, 7.40 Hz, 2H).

LCMS: 481.4 (M+H). Method: Atlantis dC18 (50×4.6) 5 μm, Mobile phase A: 0.1% FA in H₂O, Mobile phase B: ACN, Flow Rate: 1.5 mL/min. Rt (min): 1.790, Area (%): 99.565.

HPLC: Method: Mobile Phase A: 0.1% TFA in water, Mobile Phase B: ACN, Flow Rate: 2.0 mL/min. Column: X-Bridge C8 (50×4.6) mm, 3.5 μm. Rt (min): 2.778, Area (%): 99.947.

### Compound 323

The compound having the following structure is prepared in analogy from the preparation procedure described for the above-mentioned compounds.

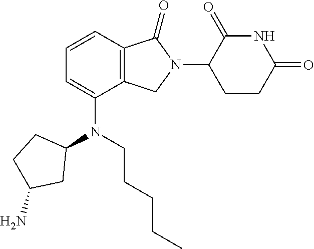 [SMILES: CCCCCN(c1cccc2c1CN(C1CCC(=O)NC1=O)C2=O)[C@@H]1CC[C@@H](N)C1]

### Compound 324

The compound having the following structure is prepared in analogy from the preparation procedure described for the above-mentioned compounds.

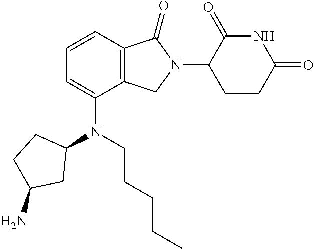 [SMILES: CCCCCN(c1cccc2c1CN(C1CCC(=O)NC1=O)C2=O)[C@@H]1CC[C@H](N)C1]

### Hydrochloride Salt Form of Compound 325

### 3-(4-(((1R,3R)-3-(aminomethyl)cyclopentyl)(pentyl)amino)-1-oxoisoindolin-2-yl)piperidine-2,6-dione HCl salt

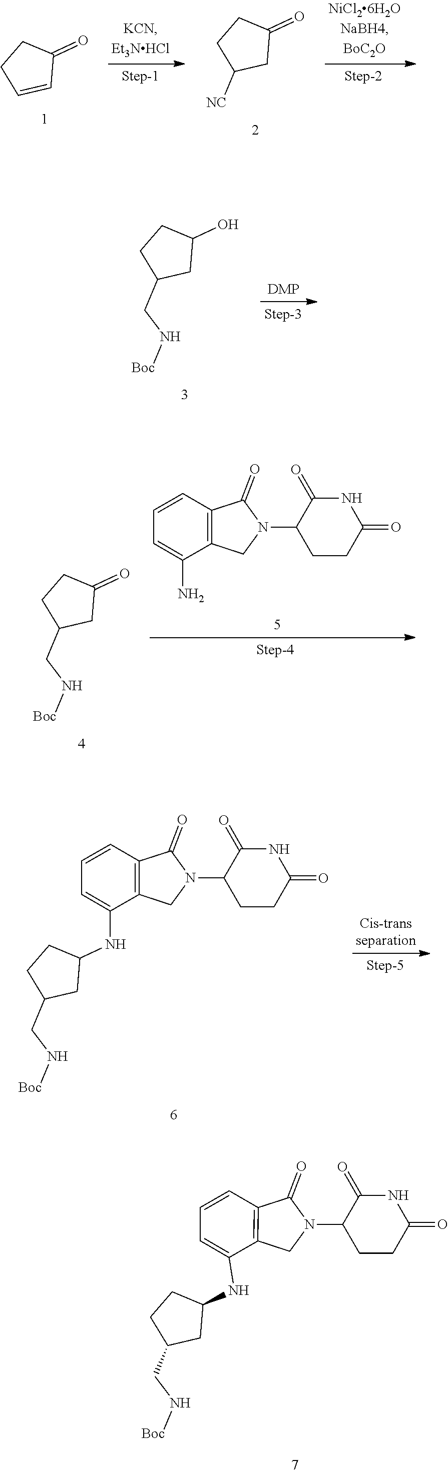

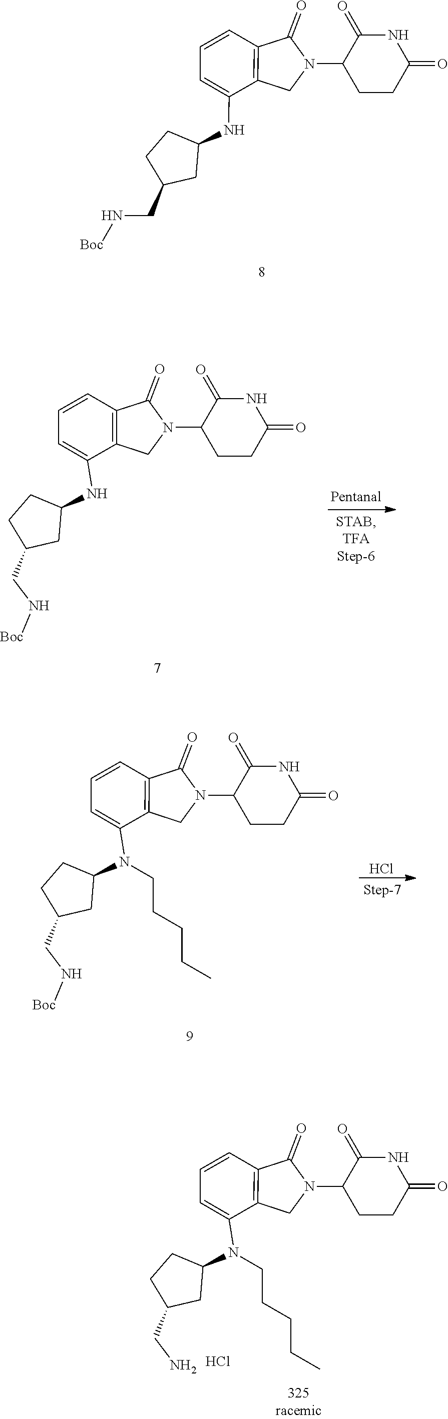 [SMILES: CC(C)(C)OC(=O)NC[C@H]1CC[C@@H](Nc2cccc3c2CN(C2CCC(=O)NC2=O)C3=O)C1 ; CC(C)(C)OC(=O)NC[C@@H]1CC[C@@H](Nc2cccc3c2CN(C2CCC(=O)NC2=O)C3=O)C1 ; CCCCCN(c1cccc2c1CN(C1CCC(=O)NC1=O)C2=O)[C@@H]1CC[C@@H](CNC(=O)OC(C)(C)C)C1 ; Cl ; CCCCCN(c1cccc2c1CN(C1CCC(=O)NC1=O)C2=O)[C@@H]1CC[C@@H](CN)C1 ; Cl]

### Step-1: 3-oxocyclopentane-1-carbonitrile

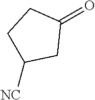

Potassium cyanide (4.76 g, 73.1 mmol, 1.2 equiv) and trimethylamine hydrochloride (10.06 g, 73.1 mmol, 1.2 equiv) were dissolved in a mixture of MeOH (15 ml) and water (10 ml). A solution of cyclopent-2-en-1-one 1 (5.0 g, 60.9 mmol, 1.0 equiv) in MeOH (15.00 ml) was added drop wise over 30 min at room temperature. The reaction mixture was stirred at room temperature for 2 h (as confirmed by TLC analysis, 20% EtOAc, Rf˜0.2), The reaction mixture was quenched with water (50 ml) and extracted with DCM (2×100 ml). The combined organic layer was washed with 1.5N HCl solution (2×50 ml) followed by water (50 ml) and dried over Na2SO4 and concentrated under vacuum to afford 3-oxocyclopentane-1-carbonitrile 2 (4.6 g, 32.4 mmol, 53.2% yield) as a pale yellow liquid. The crude as such taken for next step without purification. 1H-NMR (400 MHz, CDCl3): δ 3.18-3.23 (m, 1H), 2.50-2.59 (m, 4H), 2.36-2.45 (m, 2H) GCMS: 109 (M). Rt: 6.168 min, Area: 76.911%, Method: HP5_LOW TEM F|D. amx

### Step-2: tert-butyl ((3-hydroxycyclopentyl)methyl)carbamate

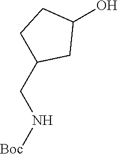 [SMILES: CC(C)(C)OC(=O)NCC1CCC(O)C1]

To a stirred solution of 3-oxocyclopentane-1-carbonitrile 2 (5.0 g, 45.8 mmol, 1.0 equiv) in methanol (100 ml), cooled to 0° C., were added (Boc)₂O (15.00 g, 68.7 mmol, 1.5 equiv), NiCl₂ 6H₂O (10.89 g, 45.8 mmol, 1.0 equiv) and sodium borohydride (12.13 g, 321 mmol, 7.0 equiv) was then carefully added in small portions (the reaction was exothermic and effervescent). The resulting reaction mixture was warmed room temperature and stirred for overnight (as confirmed by TLC analysis, 60% EtOAc, Rf˜0.3), the reaction mixture was quenched with water (100 ml) and extracted with ethyl acetate (2×100 ml). The combined organic layer was dried over Na₂SO₄ and concentrated under reduced pressure to afford a colorless liquid. The crude product was purified by Isolera (column size: Biotage R snap cartridge, KP-Sil, 250 g, 100-200 mesh, Si gel) using 25-30% EtOAc in pet-ether to afford tert-butyl ((3-hydroxycyclopentyl)methyl)carbamate 3 (5.5 g, 25.4 mmol, 55.5% yield) as a pale yellow liquid.

1H-NMR (400 MHz, DMSO-d6): δ 6.79 (t, J=7.20 Hz, 1H), 4.33 (d, J=Hz, 1H), 4.02-4.07 (m, 1H), 2.79-2.91 (m, 1H), 1.90-1.99 (m, 1H), 1.69-1.89 (m, 1H), 1.51-1.59 (m, 2H), 1.37-1.44 (m, 12H), 1.06-1.20 (m, 1H)

LCMS: 116.2 (M-Boc). Rt: 1.854-1.889 min, Area: 99.552%, Method: Column: Atlantis dC18 (50×4.6) 5, Mobile phase: A: 0.1% Formic Acid in H₂O B: Acetonitrile, Flow Rate: 1.5 ml/min.

### Step-3: tert-butyl ((3-oxocyclopentyl)methyl)carbamate

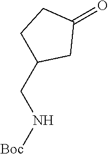 [SMILES: CC(C)(C)OC(=O)NCC1CCC(=O)C1]

To a stirred solution of tert-butyl ((3-hydroxycyclopentyl)methyl)carbamate 3 (2 g, 9.29 mmol, 1.0 equiv) in CH₂Cl₂ (50 ml), cooled to 0° C., were added Dess-Martin periodinane (13.79 g, 32.5 mmol, 3.5 equiv) and the resulting reaction mixture was warmed to room temperature and stirred for overnight. Upon completion of the reaction (as confirmed by TLC analysis, 60% EtOAc in pet-ether, Rf˜0.2), the reaction mixture was quenched with a solution of Na₂S₂O₃/NaHCO₃ (1:1, 50 ml) and stirred for 10 min. The organic layer was separated and the aqueous layer was extracted with DCM (2×100 ml). The organic layer was dried over anhydrous sodium sulphate, filtered and concentrated under vacuum to get crude as brown liquid. The crude product was purified by Isolera (column size: Biotage R snap cartridge, KP-Sil, 10 g, 230-400 mesh, Si gel) using 20-30% EtOAc in pet-ether to afford tert-butyl ((3-oxocyclopentyl)methyl)carbamate 4 (1.1 g, 5.06 mmol, 54.5% yield) as light green liquid.

1H-NMR (400 MHz, DMSO-d6): δ 6.96 (t, J=5.60 Hz, 1H), 2.20-2.21 (m, 2H), 2.10-2.12 (m, 4H), 1.95-1.97 (m, 2H), 1.94-1.94 (m, 1H), 1.84-1.86 (m, 9H),

LCMS: 114.2 (M-Boc). Rt: 1.951 min, Area: 98.222%, Method: Column: Atlantis dC18 (50×4.6) 5 Mobile phase: A: 0.1% Formic Acid in H₂O B: ACN Flow Rate: 1.5 ml/min

### Step 4 & 5

### Compound 7: tert-butyl (((1R,3R)-3-((2-(2,6-dioxopiperidin-3-yl)-1-oxoisoindolin-4-yl)amino)cyclopentyl)methyl)carbamate

### Compound 8: tert-butyl (((1S,3R)-3-((2-(2,6-dioxopiperidin-3-yl)-1-oxoisoindolin-4-yl)amino)cyclopentyl)methyl)carbamate

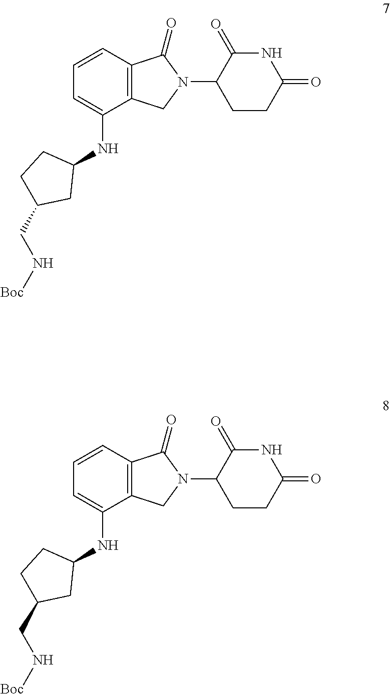 [SMILES: CC(C)(C)OC(=O)NC[C@@H]1CC[C@@H](Nc2cccc3c2CN(C2CCC(=O)NC2=O)C3=O)C1 ; CC(C)(C)OC(=O)NC[C@H]1CC[C@@H](Nc2cccc3c2CN(C2CCC(=O)NC2=O)C3=O)C1]

To a stirred solution of 3-(4-amino-1-oxoisoindolin-2-yl)piperidine-2,6-dione 5 (0.7 g, 2.70 mmol, 1.0 equiv), tert-butyl ((3-oxocyclopentyl)methyl)carbamate 4 (1.152 g, 5.40 mmol, 2.0 equiv) in DCE (10 ml)): DMF (10 ml) was added TFA (0.312 ml, 4.05 mmol, 1.5 equiv) and sodium triacetoxyhydroborate (0.858 g, 4.05 mmol, 1.5 equiv) at 0° C. The resulting reaction mixture was stirred at room temperature for overnight. Upon completion of the reaction (as confirmed by LCMS analysis), the reaction mixture was diluted with water (50 mL) and extracted with DCM (30 ml). The organic layer was separated and the aqueous layer was extracted with DCM (2×50 mL). The combined organic layer was washed with brine (1×50 mL), dried over Na₂SO₄, filtered and concentrated under reduced pressure to afford the crude tert-butyl ((3-((2-(2,6-dioxopiperidin-3-yl)-1-oxoisoindolin-4-yl)amino)cyclopentyl)methyl)carbamate (1.2 g, 2.63 mmol, 97% yield) as brown gummy liquid. The crude product was purified by preparative HPLC (X Select C18-250×19.1 mm, Mobile phase—0.1% FA in Water/ACN, Flow rate—15 mL/min), and Peak-1 was lyophilized to afford tert-butyl (((1R,3R)-3-((2-(2,6-dioxopiperidin-3-yl)-1-oxoisoindolin-4-yl)amino)cyclopentyl)methyl)carbamate 7 (0.3 g, 0.581 mmol, 22.12% yield) as brown solid.

¹H-NMR (400 MHz, DMSO-d6): δ 11.03 (s, 1H), 7.28 (t, J=7.60 Hz, 1H), 6.92 (t, J=7.60 Hz, 2H), 6.77 (d, J=8.00 Hz, 1H), 5.56 (s, 3H), 5.10-5.12 (m, 1H), 4.16-4.20 (m, 2H), 3.86 (t, J=6.00 Hz, 1H), 2.87-2.89 (m, 1H), 2.50-2.51 (m, 3H), 2.31-2.50 (m, 1H), 2.19-2.26 (m, 3H), 2.01-2.02 (m, 3H), 1.79-1.81 (m, 1H), 1.62-1.63 (m, 2H), 1.50-1.52 (m, 1H), 1.32 (s, 9H), 1.22-1.23 (m, 1H),

LCMS: 401.3 (M-t-Bu). Rt: 1.930 min, Area: 88.467%, Method: Column: X Bridge C8 (50×4.6 mm) 3.5 μm, Mobile phase: A:0.1% TFA in H₂O, Mobile phase: B: 0.1% TFA in ACN Flow Rate: 1.5 ml/min.

HPLC: Method: X-Bridge C18 (150×4.6) mm, 3.5 μm, Mobile phase: A: 0.1% TFA in water, Mobile phase: B: CAN, Flow: 1.0 mL/min, RT: 9.171-9.228 min, Area:90.617%.

The peak-2 was lyophilized to afford tert-butyl (((1S,3R)-3-((2-(2,6-dioxopiperidin-3-yl)-1-oxoisoindolin-4-yl)amino)cyclopentyl)methyl)carbamate 8 (0.32 g, 0.637 mmol, 24.22% yield) as a brown solid.

¹H-NMR (400 MHz, DMSO-d6): δ 11.03 (s, 1H), 7.28 (t, J=7.60 Hz, 1H), 6.92 (t, J=7.60 Hz, 2H), 6.77 (d, J=8.00 Hz, 1H), 5.09-5.16 (m, 1H), 4.10-4.27 (m, 2H), 3.82 (t, J=6.00 Hz, 1H), 2.91-2.97 (m, 3H), 2.51-2.64 (m, 1H), 2.14-2.22 (m, 2H), 2.00-2.07 (m, 3H), 1.64-1.70 (m, 1H), 1.48-1.55 (m, 1H), 1.36 (s, 9H), 1.13-1.20 (m, 1H),

LCMS: 401.1 (M-tBu). Rt: 1.783 min, Area: 90.82%, Method: X Bridge C8 (50×4.6 mm) 3.5 μm, Mobile phase: A: 0.1% TFA in H₂O, Mobile phase: B: 0.1% TFA in CAN, Flow Rate: 1.5 ml/min

HPLC: Method: X-Bridge C18 (150×4.6) mm, 3.5 μm, Mobile phase: A:0.1% TFA in water, Mobile phase: B: CAN, Flow:1.0 mL/min, RT: 9.496 min, Area:93.634%.

### Step-6: tert-butyl (((1R,3R)-3-((2-(2,6-dioxopiperidin-3-yl)-1-oxoisoindolin-4-yl)(pentyl)amino)cyclopentyl)methyl)carbamate

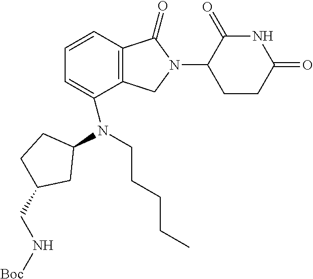 [SMILES: CCCCCN(c1cccc2c1CN(C1CCC(=O)NC1=O)C2=O)[C@@H]1CC[C@@H](CNC(=O)OC(C)(C)C)C1]

To a stirred solution of tert-butyl (((1R,3R)-3-((2-(2,6-dioxopiperidin-3-yl)-1-oxoisoindolin-4-yl)amino)cyclopentyl)methyl)carbamate 7 (0.2 g, 0.438 mmol, 1.0 equiv), pantanal (0.755 g, 8.76 mmol, 20 equiv) in DCM (3 ml): DMF (3 ml) was added TFA (0.135 ml, 1.752 mmol, 4.0 equiv) and stirred at RT for 1 h. To the reaction mixture, sodium triacetoxyborohydride (0.557 g, 2.63 mmol, 6.0 equiv) was added at 0° C. The resulting reaction mixture was stirred at room temperature for overnight. Upon completion of the reaction (as confirmed by LCMS analysis). The reaction mixture was quenched with water (50 mL) and extracted with DCM (30 ml). The organic layer was separated and the aqueous layer was extracted with DCM (2×30 mL). The combined organic layer was washed with brine (1×50 mL), dried over Na₂SO₄, filtered and concentrated under reduced pressure to afford the crude product as yellow gummy liquid. The crude product was purified by prep-HPLC (X Select C18-250×19.1 mm, Mobile phase—0.1% FA in Water/ACN, Flow rate—15 mL/min), and the pure fraction was lyophilized to tert-butyl (((1R,3R)-3-((2-(2,6-dioxopiperidin-3-yl)-1-oxoisoindolin-4-yl)(pentyl)amino)cyclopentyl)methyl)carbamate 9 (18 mg, 0.034 mmol, 7.77% yield) as a yellow solid.

¹H-NMR (400 MHz, DMSO-d6): δ 10.98 (m, 1H), 7.42 (t, J=10.00 Hz, 1H), 7.32 (t, J=10.40 Hz, 2H), 6.85 (t, J=7.60 Hz, 1H), 5.13 (s, 2H), 4.39-5.10 (m, 1H), 4.26-4.39 (m, 1H), 3.03-3.78 (m, 1H), 2.73-2.80 (m, 2H), 2.50-2.61 (m, 3H), 1.99-2.08 (m, 2H), 1.72-1.78 (m, 2H), 1.45-1.56 (m, 3H), 1.41 (s, 9H), 1.18-1.36 (m, 8H), 0.78 (t, J=9.20 Hz, 3H).

LCMS: 527.3 (M+H). Rt: 2.441 min, Area: 99.571%, Method: Atlantis dC18 (50×4.6 mm) 5 μm, Mobile phase: A: 0.1% FA in H₂O, Mobile phase: B: Acetonitrile, Flow Rate: 1.5 ml/min

HPLC: Method: X Bridge C8 (50×4.6) mm, 3.5 μm, Mobile phase: A: 0.1% TFA in water, Mobile phase: B: CAN, Flow:1.0 mL/min, RT: 3.577 min, Area:99.827%.

### Step-7: 3-(4-(((1R,3R)-3-(aminomethyl)cyclopentyl)(pentyl)amino)-1-oxoisoindolin-2-yl)piperidine-2,6-dione, HCl

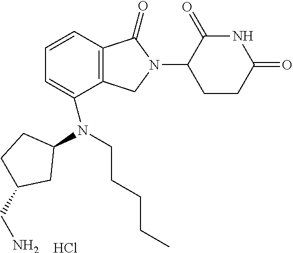 [SMILES: CCCCCN(c1cccc2c1CN(C1CCC(=O)NC1=O)C2=O)[C@@H]1CC[C@@H](CN)C1 ; Cl]

To a stirred solution of tert-butyl (((1R,3R)-3-((2-(2,6-dioxopiperidin-3-yl)-1-oxoisoindolin-4-yl)(pentyl)amino)cyclopentyl)methyl)carbamate (18 mg, 0.034 mmol) in DCM (2 ml) was added HCl (4 M soln., in ethyl acetate 2 ml, 0.034 mmol) and Stirred at RT for 1 h. Upon completion of the reaction (as confirmed by TLC analysis, 100% EtOAc, Rf˜0.1), the reaction mixture was concentrated under vacuum, the resulting residue was titrated with MTBE (2×10 ml) and decanted. The resulting residue was lyophilized to afford 3-(4-(((1R,3R)-3-(aminomethyl)cyclopentyl)(pentyl)amino)-1-oxoisoindolin-2-yl)piperidine-2,6-dione, HCl 325 (15 mg, 0.034 mmol, 94% yield) as off white solid.

¹H-NMR (400 MHz, DMSO-d6): 11.01 (s, 1H), 7.94 (s, 4H), 7.36-7.45 (m, 2H), 5.13 (d, J=10.00 Hz, 1H), 4.37-4.40 (m, 2H), 2.89-2.96 (m, 3H), 2.67-2.68 (m, 2H), 2.56-2.60 (m, 2H), 2.45-2.45 (m, 1H), 2.33-2.36 (m, 2H), 1.91-2.08 (m, 2H), 1.67-1.86 (m, 3H), 1.24-1.35 (m, 6H), 0.77-0.78 (m, 3H).

LCMS: 427.3 (M+H). Rt: 1.611 min, Area: 99.699%, Method: Column: Atlantis dC18 (50×4.6 mm) 5 m, Mobile phase: A: 0.1% FA in H₂O, Mobile phase: B: Acetonitrile, Flow Rate: 1.5 ml/min.

HPLC: Method: X Bridge C8 (50×4.6) mm, 3.5 μm, Mobile phase: A: 0.1% TFA in water, Mobile phase: B: CAN, Flow:1.0 mL/min, RT: 2.232 min, Area: 99.441%.

### Hydrochloride Salt Form of Compound 325A

### 3-(4-(((1R,3R)-3-(aminomethyl)cyclopentyl)(2-cyclopropylethyl)amino)-1-oxoisoindolin-2-yl)piperidine-2,6-dione, HCl

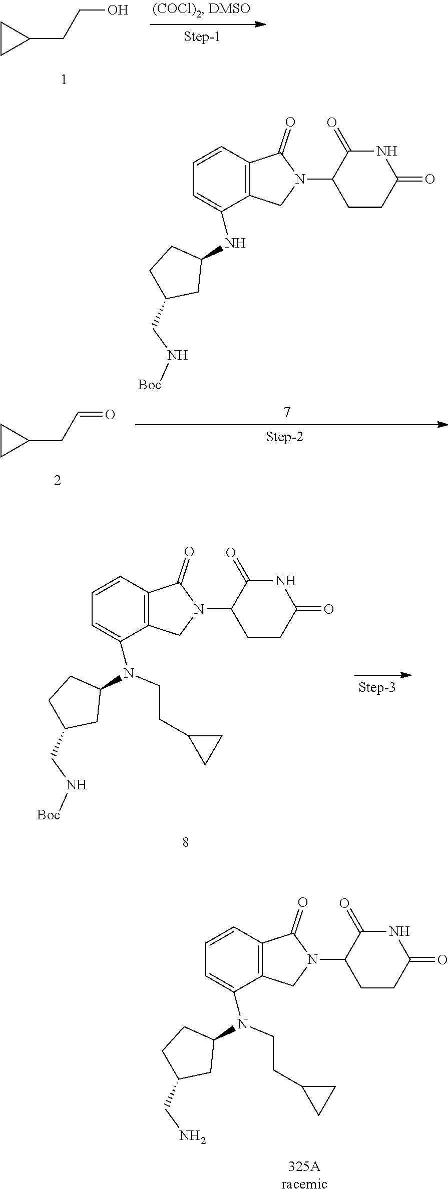 [SMILES: OCCC1CC1 ; CC(C)(C)OC(=O)NC[C@@H]1CC[C@@H](Nc2cccc3c2CN(C2CCC(=O)NC2=O)C3=O)C1 ; O=CCC1CC1 ; CC(C)(C)OC(=O)NC[C@@H]1CC[C@@H](N(CCC2CC2)c2cccc3c2CN(C2CCC(=O)NC2=O)C3=O)C1 ; NC[C@@H]1CC[C@@H](N(CCC2CC2)c2cccc3c2CN(C2CCC(=O)NC2=O)C3=O)C1]

### Step-1: 2-cyclopropylacetaldehyde (2)

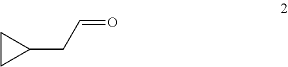

To a solution of oxalyl chloride (2.389 ml, 27.9 mmol, 2 equiv) in DCM (10 ml) at −78° C. was added DMSO (3.96 ml, 55.7 mmol, 4 equiv) dropwise and was stirred for 30 min at the same temperature. A solution of 2-cyclopropylethan-1-ol 1 (1.2 g, 13.93 mmol, 1 equiv) in DCM (5 ml) was added and the resulting solution was stirred at −78° C. for 1.5 h. Then TEA (11.59 ml, 84 mmol, 6 equiv) was added and the reaction mixture was slowly warmed to 0° C. and stirred for 1 h. Upon completion of the reaction (as confirmed by TLC; 10% EtOAc in pet ether, R_{f}: 0.6, stained by KMnO₄) the reaction mixture was quenched with ice cold aq. HCl solution (1N soln., 5 mL) at 0° C. and extracted with DCM (2×20 mL). The combined organic layer was washed with saturated aqueous NaHCO₃ solution (1×30 mL) followed by brine (1×20 mL), dried over Na₂SO₄, filtered and concentrated under vacuum at 0° C. to half of the volume to afford the crude of 2-cyclopropylacetaldehyde 2 (1 g, 11.89 mmol, 85% yield) as pale yellow liquid which was used for the next step.

¹H-NMR (400 MHz, DMSO-d6): δ 9.70 (s, 1H), 2.42 (s, 2H), 1.34 (t, J=7.20 Hz, 2H), 0.52-0.50 (m, 2H), 0.50-0.40 (m, 3H), 0.40-0.38 (m, 3H).

### Step-2: tert-butyl (((1R,3R)-3-((2-cyclopropylethyl)(2-(2,6-dioxopiperidin-3-yl)-1-oxoisoindolin-4-yl)amino)cyclopentyl)methyl)carbamate (8)

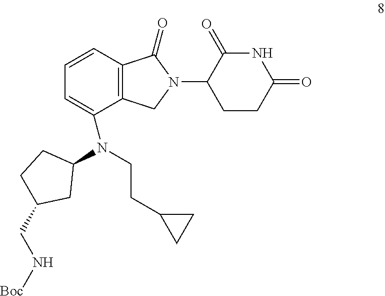 [SMILES: CC(C)(C)OC(=O)NC[C@@H]1CC[C@@H](N(CCC2CC2)c2cccc3c2CN(C2CCC(=O)NC2=O)C3=O)C1]

To a mixture of tert-butyl (((1R,3R)-3-((2-(2,6-dioxopiperidin-3-yl)-1-oxoisoindolin-4-yl)amino)cyclopentyl)methyl)carbamate 7 (0.250 g, 0.548 mmol, 1 equiv) and 2-cyclopropylacetaldehyde 2 (0.461 g, 5.48 mmol, 10 equiv) in a mixture of DCM (5 ml) and DMF (5 ml) was added TFA (0.169 ml, 2.190 mmol, 4 equiv) and stirred at room temperature for 1 h. Then STAB (0.696 g, 3.29 mmol, 6 equiv) was added at 0° C. in portions and the resulting reaction mixture was stirred at room temperature for 16 h. Upon completion of the reaction (as confirmed by LCMS), the reaction mixture was quenched with water (50 mL) and extracted with DCM (3×30 ml). The combined organic layer was washed with brine (1×50 mL), dried over Na₂SO₄, filtered and concentrated under vacuum to afford the crude product which was purified by preparative HPLC (XSelect C18-250×19.1 mm, Mobile phase—0.1% FA in Water/ACN, Flow rate—15 mL/min) to afford tert-butyl (((1R,3R)-3-((2-cyclopropylethyl)(2-(2,6-dioxopiperidin-3-yl)-1-oxoisoindolin-4-yl)amino)cyclopentyl)methyl)carbamate 8 (40 mg, 0.075 mmol, 15.84% yield) as white solid.

¹H-NMR (400 MHz, DMSO-d6): δ 10.96 (s, 1H), 7.41 (t, J=10.40 Hz, 1H), 7.30 (t, J=12.00 Hz, 2H), 6.83 (d, J=6.40 Hz, 1H), 5.10 (t, J=3.60 Hz, 1H), 4.37 (d, J=22.80 Hz, 1H), 4.24 (d, J=23.60 Hz, 1H), 3.79 (d, J=8.00 Hz, 1H), 3.15 (s, 2H), 2.83 (t, J=8.80 Hz, 2H), 2.12-1.99 (m, 2H), 1.75 (t, J=26.00 Hz, 2H), 1.54 (t, J=10.00 Hz, 3H), 1.36 (s, 10H), 1.17 (q, J=16.40 Hz, 4H), 0.57 (d, J=7.60 Hz, 1H), 0.33 (t, J=9.60 Hz, 2H), −0.07 (d, 2H).

LCMS: 525.1 (M+H), Rt (min): 2.353 min, Area (%): 99.744. HPLC: Rt (min): 4.035, Area (%): 99.065.

### Step-3: 3-(4-(((1R,3R)-3-(aminomethyl)cyclopentyl)(2-cyclopropylethyl)amino)-1-oxoisoindolin-2-yl)piperidine-2,6-dione, HCl (325A)

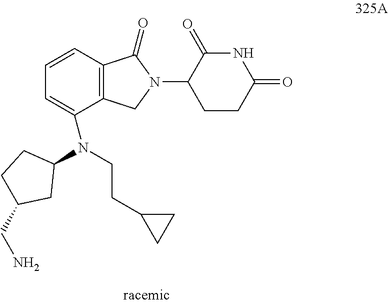 [SMILES: NC[C@@H]1CC[C@@H](N(CCC2CC2)c2cccc3c2CN(C2CCC(=O)NC2=O)C3=O)C1]

To a solution of tert-butyl (((1R,3R)-3-((2-cyclopropylethyl)(2-(2,6-dioxopiperidin-3-yl)-1-oxoisoindolin-4-yl)amino)cyclopentyl)methyl)carbamate 8 (40 mg, 0.076 mmol) in DCM (2 ml) was added HCl (4M soln. in dioxane, 10 ml, 40.0 mmol) at 0° C. and was allowed to stir at room temperature for 2 h. Upon completion of reaction (as confirmed by 100% ethyl acetate, R_{f}: 0.0), the reaction mixture was concentrated under reduced pressure, the resulting residue was diluted with water (50 ml) and washed with DCM (2×5 ml). The aqueous layer was lyophilized to get 3-(4-(((1R,3R)-3-(aminomethyl)cyclopentyl)(2-cyclopropylethyl)amino)-1-oxoisoindolin-2-yl)piperidine-2,6-dione, HCl 325A (35 mg, 0.076 mmol, 99% yield) as pale yellow solid.

¹H-NMR (400 MHz, DMSO-d6): δ 11.00 (s, 1H), 7.81 (s, 3H), 7.31-7.45 (m, 3H), 5.11-5.14 (m, 1H), 4.44-4.46 (m, 2H), 3.74 (m, 1H), 3.50-3.50 (m, 1H), 3.47-3.48 (m, 1H), 3.39-3.42 (m, 2H), 2.90-3.17 (m, 1H), 2.67-2.73 (m, 2H), 2.61-2.67 (m, 1H), 2.50-2.51 (m, 1H), 2.32-2.34 (m, 1H), 1.50-2.04 (m, 4H), 1.17-1.24 (m, 3H), 0.58-0.59 (m, 1H), 0.33-0.35 (m, 2H), −0.06 (m, 2H).

LCMS: 425.0 (M+H). Method: Column: Atlantis dC18 (50×4.6 mm) 5 μm, Mobile phase: A: 0.1% FA in H₂O, Mobile phase: B: Acetonitrile, Flow Rate: 1.5 ml/min, Rt (min): 1.52, Area (%): 99.76.

HPLC: Method: Mobile phase: A:0.1% FA in water, Mobile phase: B: ACN, Flow rate:2.0 ml/min. Column: X-Bridge C8(50×4.6) mm, 3.5 μm; Rt (min): 1.964, Area (%): 99.91.

### Formic Acid salt form of Compound 326

### 3-(4-(((1R,3S)-3-(aminomethyl)cyclopentyl)(pentyl)amino)-1-oxoisoindolin-2-yl)piperidine-2,6-dione, Formic Acid salt

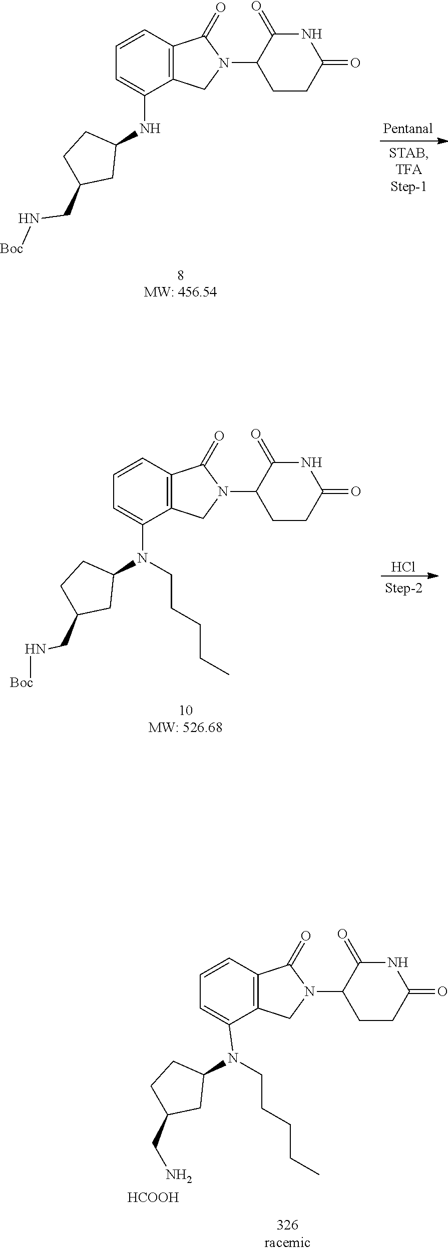 [SMILES: CC(C)(C)OC(=O)NC[C@H]1CC[C@@H](Nc2cccc3c2CN(C2CCC(=O)NC2=O)C3=O)C1 ; CCCCCN(c1cccc2c1CN(C1CCC(=O)NC1=O)C2=O)[C@@H]1CC[C@H](CNC(=O)OC(C)(C)C)C1 ; Cl ; CCCCCN(c1cccc2c1CN(C1CCC(=O)NC1=O)C2=O)[C@@H]1CC[C@H](CN)C1 ; "HCOOH"]

### Step-1: tert-butyl (((1S,3R)-3-((2-(2,6-dioxopiperidin-3-yl)-1-oxoisoindolin-4-yl)(pentyl)amino)cyclopentyl)methyl)carbamate

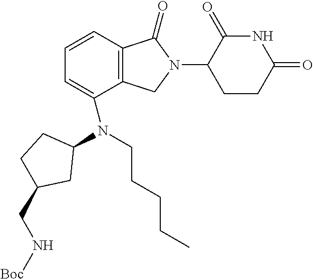 [SMILES: CCCCCN(c1cccc2c1CN(C1CCC(=O)NC1=O)C2=O)[C@@H]1CC[C@H](CNC(=O)OC(C)(C)C)C1]

To a stirred solution of tert-butyl (((1S,3R)-3-((2-(2,6-dioxopiperidin-3-yl)-1-oxoisoindolin-4-yl)amino)cyclopentyl)methyl)carbamate 8 (0.3 g, 0.657 mmol, 1.0 equiv), pentanal (1.132 g, 13.14 mmol, 20 equiv) in a mixture of DCM (4 ml) and DMF (4 ml) was added TFA (0.203 ml, 2.63 mmol, 4.0 equiv) and the mixture was stirred at RT for 1 h. To the reaction mixture, sodiumtriacetoxyborohydride (0.836 g, 3.94 mmol, 6.0 equiv) was added at 0° C. The resulting reaction mixture was stirred at room temperature for overnight. Upon completion of the reaction (as confirmed by LCMS analysis), the reaction mixture was quenched with water (50 mL) and extracted with DCM (30 ml). The organic layer was separated and the aqueous layer was extracted with DCM (2×30 mL). The combined organic layer was washed with brine (1×50 mL), dried over Na₂SO₄, filtered and concentrated. under reduced pressure to afford the crude product as yellow gummy liquid. The crude product was purified by prep-HPLC (X Select C18-250×19.1 mm, Mobile phase—0.1% FA in Water/ACN, Flow rate—15 mL/min), and the pure fraction was lyophilized to afford tert-butyl (((1S,3R)-3-((2-(2,6-dioxopiperidin-3-yl)-1-oxoisoindolin-4-yl)(pentyl)amino)cyclopentyl)methyl)carbamate 10 (0.150 g, 0.271 mmol, 41.2% yield) as a yellow solid.

1H-NMR (400 MHz, DMSO-d6): δ 11.01 (s, 1H), 7.40-7.47 (m, 3H), 6.85 (s, 1H), 5.08-5.14 (m, 2H), 4.27-4.44 (m, 3H), 3.78 (s, 1H), 3.10 (s, 2H), 2.87-2.96 (m, 3H), 2.73 (s, 1H), 1.95-2.01 (m, 2H), 1.54-1.62 (m, 4H), 1.35-1.49 (m, 19H), 1.21 (t, J=16.80 Hz, 3H),

LCMS: 527.3 (M+H). Rt: 2.428 min, Area: 95.156%, Method: Column: Atlantis dC18 (50×4.6 mm) 5 m, Mobile phase: A: 0.1% FA in H₂O, Mobile phase: B: Acetonitrile Flow Rate: 1.5 ml/min

HPLC: Method: X Bridge C8 (50×4.6) mm, 3.5 μm, Flow Rate: 2.0 ml/min, Mobile Phase A: 0.1% TFA in water, Mobile Phase B: Acetonitrile, RT: 3.483 min, Area: 97.744%.

### Step-2: 3-(4-(((1R,3S)-3-(aminomethyl)cyclopentyl)(pentyl)amino)-1-oxoisoindolin-2-yl)piperidine-2,6-dione, Formic Acid

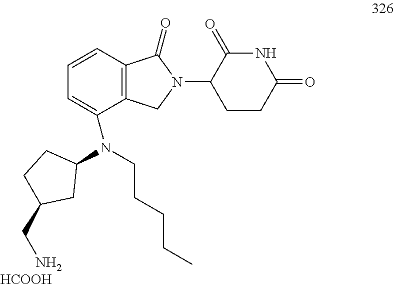 [SMILES: CCCCCN(c1cccc2c1CN(C1CCC(=O)NC1=O)C2=O)[C@@H]1CC[C@H](CN)C1 ; "HCOOH"]

To a stirred solution of tert-butyl (((1S,3R)-3-((2-(2,6-dioxopiperidin-3-yl)-1-oxoisoindolin-4-yl)(pentyl)amino)cyclopentyl)methyl)carbamate (0.150 g, 0.285 mmol) in DCM (4 ml) was added HCl (4 M soln. in ethyl acetate, 5 ml) and stirred at room temperature for 1 hour. Upon completion of the reaction (as confirmed by TLC analysis, 100% EtOAc, Rf˜0.0). The reaction mixture was concentrated under vacuum, the resulting residue was triturated with MTBE (2×10 ml) and decanted. The resulting residue was lyophilized to afford crude product as a off white solid. The crude product purified by prep-HPLC (X Select C18-250x 19.1 mm, Mobile phase −0.1% FA in Water/ACN, Flow rate—15 mL/min) to afford 3-(4-(((1R,3S)-3-(aminomethyl)cyclopentyl)(pentyl)amino)-1-oxoisoindolin-2-yl)piperidine-2,6-dione. Formic Acid 326 (42 mg, 0.088 mmol, 30.9% yield) as a off white solid.

1H-NMR (400 MHz, DMSO-d6): δ 7.78 (s, 1H), 7.44 (t, J=8 Hz, 1H), 7.37 (t, J=8.4 Hz, 2H), 5.09-5.14 (m, 1H), 4.34 (d, J=10 Hz, 1H), 4.26 (d, J=10 Hz, 1H), 3.76 (s, 1H), 3.42-3.77 (m, 2H), 2.88 (s, 2H), 2.75-2.89 (m, 1H), 2.62-2.75 (m, 2H), 2.51-2.62 (m, 1H), 2.00-2.05 (m, 3H), 1.93-1.96 (m, 2H), 1.50-1.52 (m, 1H), 1.36-1.34 (m, 2H), 1.23-1.18 (m, 7H), 0.80 (t, J=8 Hz, 3H).

LCMS: 427.2 (M+H). Rt: 1.210 min, Area: 99.163%, Method: Column: X Bridge C8 (50×4.6 mm) 3.5 μm, Mobile phase: A: 0.1% TFA in H₂O, Mobile phase: B: 0.1% TFA in CAN, Flow Rate: 1.5 ml/min

HPLC: Method: X Bridge C8 (50×4.6) mm, 3.5 μm, Flow Rate: 2.0 ml/min, Mobile Phase A: 0.1% FA in water, Mobile Phase B: Acetonitrile, RT: 2.006 min, Area: 96.918%.

### Hydrochloride Salt Form of Compound 326A

### 3-(4-(((1R,3S)-3-(aminomethyl)cyclopentyl)(2-cyclopropylethyl)amino)-1-oxoisoindolin-2-yl)piperidine-2,6-dione, HCl

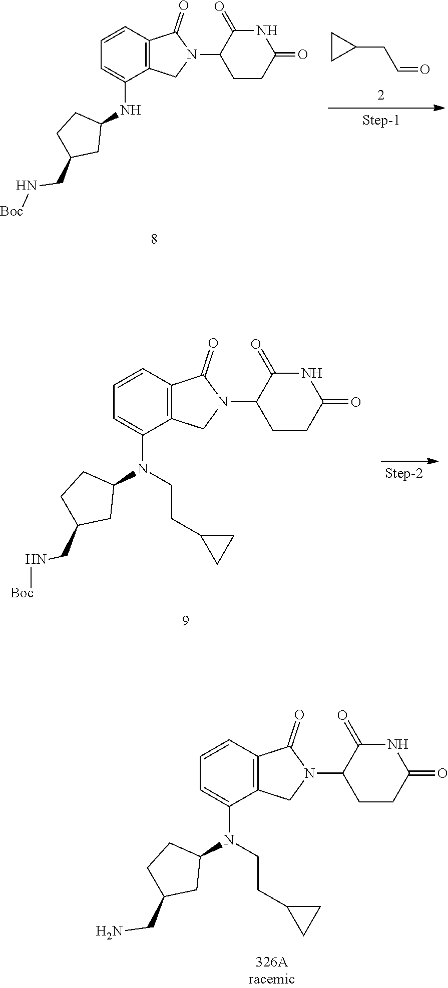

### Step-1: tert-butyl (((1S,3R)-3-((2-cyclopropylethyl)(2-(2,6-dioxopiperidin-3-yl)-1-oxoisoindolin-4-yl)amino)cyclopentyl)methyl)carbamate (9)

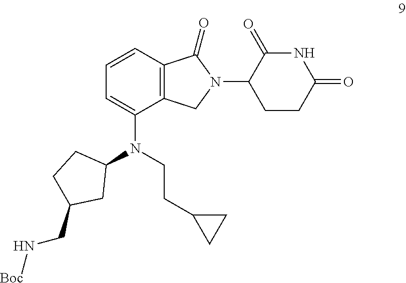 [SMILES: CC(C)(C)OC(=O)NC[C@H]1CC[C@@H](N(CCC2CC2)c2cccc3c2CN(C2CCC(=O)NC2=O)C3=O)C1]

To a mixture of tert-butyl (((1S,3R)-3-((2-(2,6-dioxopiperidin-3-yl)-1-oxoisoindolin-4-yl)amino)cyclopentyl)methyl)carbamate 8 (0.3 g, 0.657 mmol, 1 equiv) and 2-cyclopropylacetaldehyde 2 (0.553 g, 6.57 mmol, 10 equiv) in a mixture of DCM (5 ml) and DMF (5 ml) was added TFA (0.203 ml, 2.63 mmol, 4 equiv) and stirred at room temperature for 1 h. To this mixture, STAB (0.836 g, 3.94 mmol, 6 equiv) was added at 0° C. and the resulting reaction mixture was stirred at room temperature for 16 h. Upon completion of the reaction (as confirmed by LCMS) the reaction mixture was quenched with water (20 mL) and extracted with DCM (3×20 ml). The combined organic layer was washed with brine (1×50 mL), dried over Na₂SO₄, filtered and concentrated under vacuum to give the crude product which was purified by preparative HPLC (X Select C18-250×19.1 mm, Mobile phase—0.1% FA in Water/ACN, Flow rate—15 mL/min) to afford tert-butyl (((1S,3R)-3-((2-cyclopropylethyl)(2-(2,6-dioxopiperidin-3-yl)-1-oxoisoindolin-4-yl)amino)cyclopentyl)methyl)carbamate 9 (80 mg, 0.152 mmol, 26.5% yield) as white solid.

¹H-NMR (400 MHz, DMSO-d₆): δ 10.96 (s, 1H), 7.42 (t, J=10.00 Hz, 1H), 7.30 (t, J=12.80 Hz, 2H), 6.84 (s, 1H), 5.09 (t, J=7.20 Hz, 1H), 4.37 (d, J=22.80 Hz, 1H), 4.26 (d, J=22.40 Hz, 1H), 3.74 (s, 1H), 3.16 (t, J=8.80 Hz, 2H), 2.88 (t, J=10.00 Hz, 3H), 2.00 (t, J=8.40 Hz, 3H), 1.86 (t, J=7.60 Hz, 1H), 1.59 (d, J=10.40 Hz, 1H), 1.50 (d, J=11.20 Hz, 1H), 1.36 (d, J=2.40 Hz, 10H), 1.16 (d, J=10.00 Hz, 3H), 0.59 (s, 1H), 0.33 (q, J=7.20 Hz, 2H), −0.068 (t, 2H).

LCMS: 525.1 (M+H), Rt (min): 2.351 min, Area (%): 99.762%. HPLC: Rt (min):4.038, Area (%): 99.476.

### Step-2: 3-(4-(((1R,3S)-3-(aminomethyl)cyclopentyl)(2-cyclopropylethyl)amino)-1-oxoisoindolin-2-yl)piperidine-2,6-dione, HCl

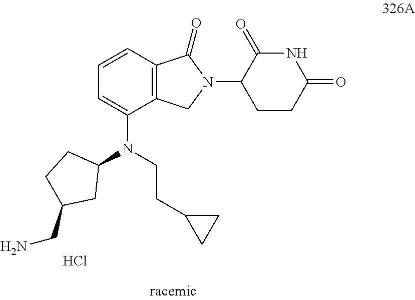 [SMILES: NC[C@H]1CC[C@@H](N(CCC2CC2)c2cccc3c2CN(C2CCC(=O)NC2=O)C3=O)C1 ; Cl]

To a solution of tert-butyl (((1S,3R)-3-((2-cyclopropylethyl)(2-(2,6-dioxopiperidin-3-yl)-1-oxoisoindolin-4-yl)amino)cyclopentyl)methyl)carbamate (80 mg, 0.152 mmol) in DCM (2 ml) was added HCl (4M soln in dioxane, 10 ml, 40.0 mmol) at 0° C. and was allowed to stir at room temperature for 2 h. Upon completion of reaction (as confirmed by TLC analysis, 100% ethyl acetate, Rf: 0.0) the reaction mixture was concentrated under vacuum and the resulting residue was diluted with water (50 ml) and washed with DCM (2×5 ml). The aqueous layer was lyophilized to get 3-(4-(((1R,3S)-3-(aminomethyl)cyclopentyl)(2-cyclopropylethyl)amino)-1-oxoisoindolin-2-yl)piperidine-2,6-dione, HCl 326 A (70 mg, 0.151 mmol, 99% yield) as pale yellow solid.

¹H-NMR (400 MHz, DMSO-d6): δ 11.01 (s, 1H), 7.86 (s, 3H), 7.85 (m, 1H), 7.36 (m, 2H), 5.12-5.14 (m, 1H), 4.32-4.56 (m, 2H), 3.42-3.43 (m, 1H), 3.39-3.41 (m, 1H), 2.96-3.19 (m, 2H), 2.90-2.93 (m, 1H), 2.79 (m, 2H), 2.64-2.68 (m, 1H), 2.50-2.51 (m, 1H), 2.09 (m, 3H), 1.50-1.76 (m, 4H), 1.21-1.36 (m, 2H), 0.59 (m, 1H), 0.33-0.35 (m, 2H), −0.05 (m, 2H).

LCMS: 425.0 (M+H). Method: Column: Atlantis dC18 (50×4.6 mm) 5 μm, Mobile phase: A: 0.1% FA in H₂O, Mobile phase: B: Acetonitrile, Flow Rate: 1.5 ml/min, Rt (min): 1.523, Area (%): 99.337.

HPLC: Method: Mobile phase: A:0.1% FA in water, Mobile phase: B: ACN, Flow rate:2.0 ml/min. Column: X-Bridge C8(50×4.6) mm, 3.5 μm; Rt (min): 1.964, Area (%): 99.566.

### Compound 327

The compound having the following structure is prepared in analogy from the preparation procedure described for the above-mentioned compounds.

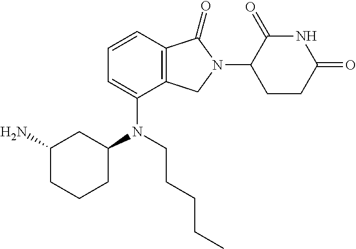 [SMILES: CCCCCN(c1cccc2c1CN(C1CCC(=O)NC1=O)C2=O)[C@H]1CCC[C@H](N)C1]

### Compound 328

The compound having the following structure is prepared in analogy from the preparation procedure described for the above-mentioned compounds.

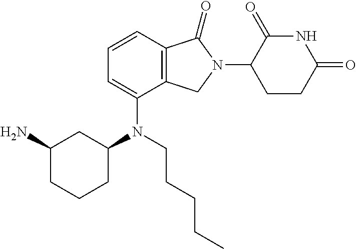 [SMILES: CCCCCN(c1cccc2c1CN(C1CCC(=O)NC1=O)C2=O)[C@H]1CCC[C@@H](N)C1]

### Hydrochloride Salt Form of Compound 329

### 3-(4-(((1r,4r)-4-aminocyclohexyl)(3-(tetrahydro-2H-pyran-4-yl)propyl)amino)-1-oxoisoindolin-2-yl)piperidine-2,6-dione, HCl

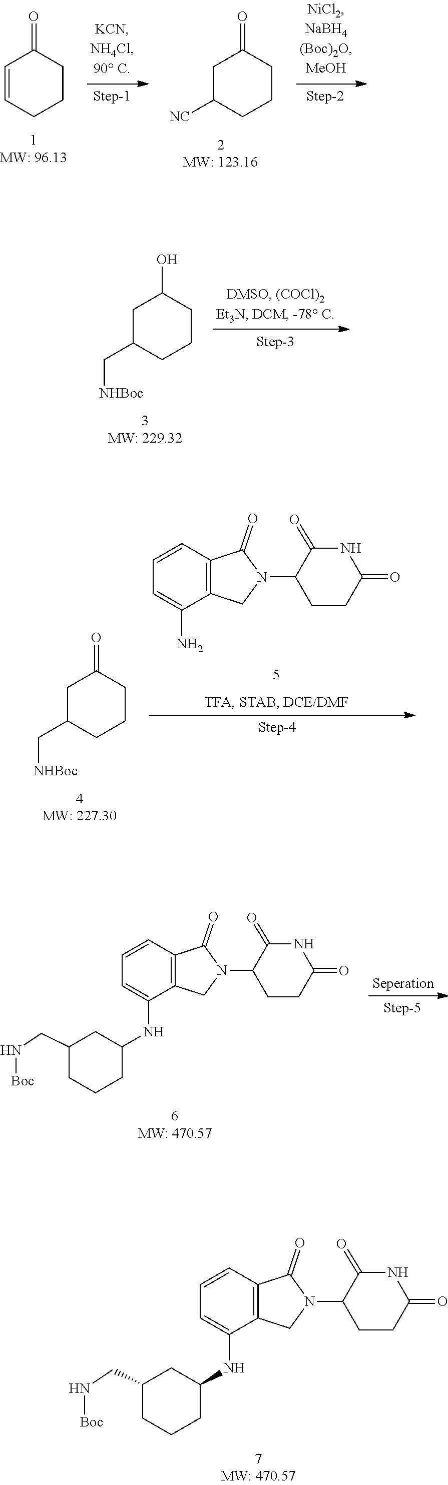

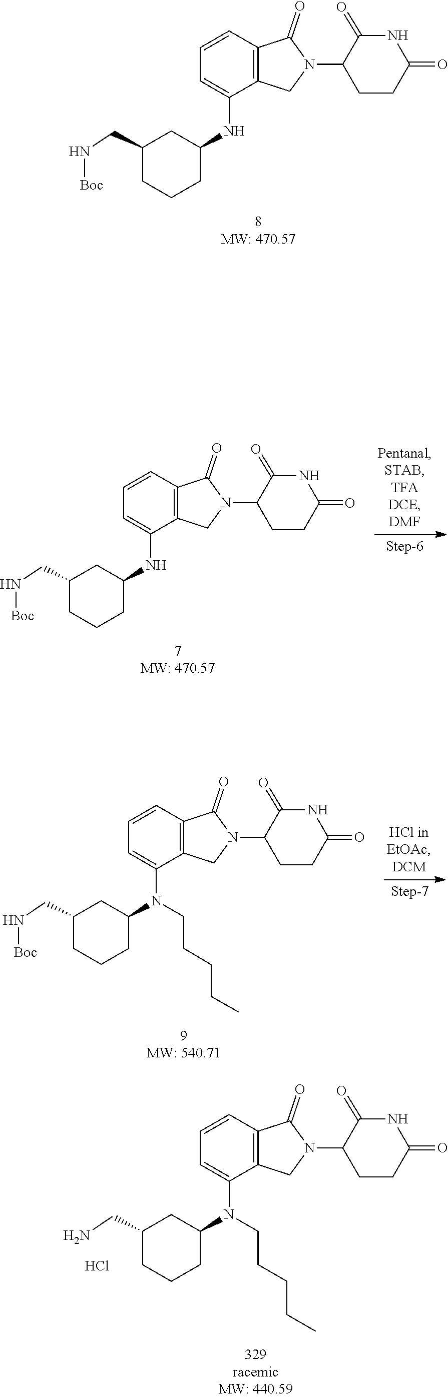 [SMILES: CC(C)(C)OC(=O)NC[C@@H]1CCC[C@H](Nc2cccc3c2CN(C2CCC(=O)NC2=O)C3=O)C1 ; CC(C)(C)OC(=O)NC[C@H]1CCC[C@H](Nc2cccc3c2CN(C2CCC(=O)NC2=O)C3=O)C1 ; CCCCCN(c1cccc2c1CN(C1CCC(=O)NC1=O)C2=O)[C@H]1CCC[C@H](CNC(=O)OC(C)(C)C)C1 ; CCCCCN(c1cccc2c1CN(C1CCC(=O)NC1=O)C2=O)[C@H]1CCC[C@H](CN)C1 ; Cl]

### Step-1: 3-oxocyclohexane-1-carbonitrile (2)

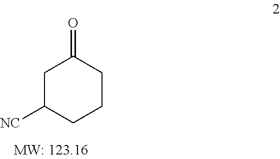

To a stirred solution of potassium cyanide (4.06 g, 62.4 mmol, 1.5 equiv) in water (30.0 mL) were added ammonium chloride (2.67 g, 49.9 mmol, 1.2 equiv) and a solution of cyclohex-2-en-1-one 1 (4.0 g, 41.6 mmol, 1.0 equiv) in DMF (30.0 ml) at room temperature over a period of 15 min. Then the reaction mixture was stirred for 4 h at 90° C. Upon completion of the reaction (as confirmed by TLC analysis, 25% EtOAc in pet ether, R_{f}˜0.1), the reaction mixture was quenched with ice cold water (150 mL) and extracted with DCM (3×50 mL). The combined organic layer was washed with brine (1×50 mL), dried over Na₂SO₄, filtered and concentrated under reduced pressure to afford the crude product which was purified by Isolera (column size: Biotage R snap cartridge, KP-Sil, 100 g, 230-400 silica gel) using 30-40% ethyl acetate in pet ether. The pure fractions were concentrated under vacuum to afford 3-oxocyclohexane-1-carbonitrile 2 (2.25 g, 16.37 mmol, 39.3% yield) as pale yellow liquid.

¹H-NMR (400 MHz, CDCl₃): δ 3.07-3.05 (m, 1H), 2.73-2.68 (m, 1H), 2.65-2.59 (m, 1H), 2.45-2.41 (m, 2H), 2.21-2.14 (m, 3H), 2.08-2.01 (m, 1H), 1.90-0.86 (m, 2H), 1.84-1.71 (m, 1H).

GCMS: 123.00 [M], Rt (min): 6.543, Area (%): 89.6057.

### Step-2: tert-butyl ((3-hydroxycyclohexyl)methyl)carbamate (3)

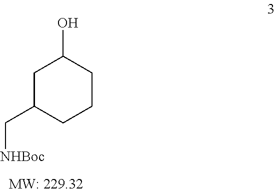 [SMILES: CC(C)(C)OC(=O)NCC1CCCC(O)C1]

To a stirred solution of 3-oxocyclohexane-1-carbonitrile 2 (1.42 g, 11.53 mmol, 1.0 equiv) in MeOH (50 ml) were added (Boc)₂O (3.97 ml, 17.30 mmol, 1.5 equiv) and NiCl₂·6H₂O (2.74 g, 11.53 mmol, 1.0 equiv) at 0° C. Then sodium borohydride (3.05 g, 81 mmol, 7.0 equiv) was added carefully in small portions over a period of 45 min (the reaction was exothermic). The resulting reaction mixture was allowed to warm to room temperature and stirred for overnight. Upon completion of the reaction (as confirmed by TLC analysis, 60% EtOAc in pet ether, R_{f}˜0.4 & LCMS-ELSD), the reaction mixture was filtered through Celite bed and washed with MeOH (2×20 mL) followed by water (1×50 mL) and concentrated under reduced pressure to afford a residue which was dissolved in EtOAc (75 mL) and washed with water (75 mL). The organic layer was separated and aqueous layer was extracted with EtOAc (2×50 mL). The combined organic layer was washed with brine (1×50 mL), dried over Na₂SO₄, filtered and concentrated under reduced pressure to afford the crude product which was purified by reverse-phase column chromatography (column: C18 40 μm, 120 g; flow rate: 20 mL/min; 0.1% aqueous HCOOH/MeCN). The pure fractions were concentrated under reduced pressure and the aqueous layer was extracted with EtOAc (2×40 mL). The combined organic layer was washed with brine (1×150 mL), dried over Na₂SO₄, filtered and concentrated under reduced pressure to get tert-butyl ((3-hydroxycyclohexyl)methyl)carbamate 3 (1.3 g, 5.61 mmol, 48.6% yield) as colorless liquid.

¹H-NMR (400 MHz, DMSO-d₆): δ 6.78 (br s, 1H), 4.47 (m, 1H), 3.31-3.27 (m, 1H), 2.79-2.72 (m, 2H), 1.78 (m, 2H), 1.67-1.62 (m, 1H), 1.56-1.52 (m, 2H), 1.37-1.31 (m, 12H), 1.14-0.96 (m, 2H), 0.76-0.64 (m, 2H).

LCMS: 130.2 [(M+H)*(Boc cleaved mass)], Rt (min): 5.427, Area (%): 82.869.

### Step-3: tert-butyl ((3-oxocyclohexyl)methyl)carbamate (4)

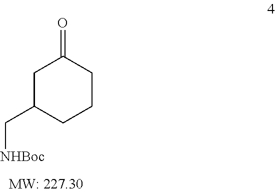 [SMILES: CC(C)(C)OC(=O)NCC1CCCC(=O)C1]

To a stirred solution of tert-butyl ((3-hydroxycyclohexyl)methyl)carbamate 3 (0.300 g, 1.308 mmol, 1.0 equiv) in DCM (2.0 mL) was added Dess-Martin periodinane (1.110 g, 2.62 mmol, 2.0 equiv) at 0° C. and stirred for 10 min. Then the reaction mixture was allowed to warm to room temperature and stirred for 3 h. Upon completion of the reaction (as confirmed by TLC analysis, 5% MeOH in DCM, R_{f}˜0.6), the reaction mixture was quenched with 1:1 of Na₂S₂O₃ and saturated NaHCO₃ solution (25 mL) and extracted with DCM (3×10 mL). The combined organic layer was washed with brine (1×50 mL), dried over Na₂SO₄, filtered and concentrated under reduced pressure to afford the crude of tert-butyl ((3-oxocyclohexyl)methyl)carbamate 4 (0.270 g, 1.186 mmol, 91% yield) as colorless liquid which was taken for next step without further purification.

¹H-NMR (400 MHz, DMSO-d₆): δ 6.90 (br s, 1H), 2.91-2.82 (m, 2H), 2.33-2.28 (m, 1H), 2.27-2.16 (m, 3H), 1.98-1.91 (m, 1H), 1.82-1.73 (m, 2H), 1.58-1.48 (m, 1H), 1.38-1.28 (m, 10H).

LCMS: 128.2 [(M+H)⁺ (Boc cleaved mass)], Rt (min): 2.070, Area (%): 99.853

### Step 4 & 5: tert-butyl (((1S,3S)-3-((2-(2,6-dioxopiperidin-3-yl)-1-oxoisoindolin-4-yl)amino)cyclohexyl)methyl)carbamate (7) & tert-butyl (((1R,3S)-3-((2-(2,6-dioxopiperidin-3-yl)-1-oxoisoindolin-4-yl)amino)cyclohexyl)methyl)carbamate (8)

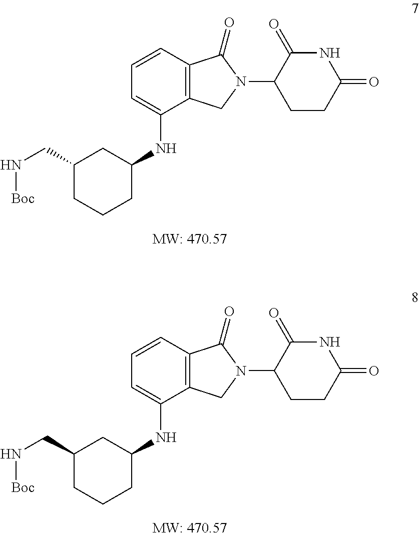 [SMILES: CC(C)(C)OC(=O)NC[C@H]1CCC[C@H](Nc2cccc3c2CN(C2CCC(=O)NC2=O)C3=O)C1 ; CC(C)(C)OC(=O)NC[C@@H]1CCC[C@H](Nc2cccc3c2CN(C2CCC(=O)NC2=O)C3=O)C1]

To a stirred solution of 3-(4-amino-1-oxoisoindolin-2-yl)piperidine-2,6-dione 5 (0.250 g, 0.964 mmol, 1.0 equiv), tert-butyl ((3-oxocyclohexyl)methyl)carbamate 4 (0.329 g, 1.446 mmol, 2.0 equiv) in a mixture of 1,2-dichloroethane (3.0 mL) and DMF (3.0 mL) was added TFA (0.074 ml, 0.964 mmol, 1.0 equiv) and STAB (0.409 g, 1.929 mmol, 1.5 equiv) at 0° C. The resulting reaction mixture was stirred at room temperature for 16 h. Upon completion of the reaction (as confirmed by LCMS analysis), the reaction mixture was poured into ice cold water (50 mL) and extracted with EtOAc (3×20 mL). The combined organic layer was washed with brine (50 mL), dried over anhy. Na₂SO₄, filtered and concentrated under vacuum to get the crude product which was purified by preparative HPLC (Column: X Bridge C18-19.1 x250 MM, 500 uL, Mobile phase: A: 0.1% TFA in water, B: MeCN, Flow rate: 15 mL/min) and lyophilized to get tert-butyl (((1S,3S)-3-((2-(2,6-dioxopiperidin-3-yl)-1-oxoisoindolin-4 yl)amino)cyclohexyl)methyl)carbamate 7 (0.130 g, 0.270 mmol, 28% yield) as pale yellow solid and tert-butyl (((1R,3S)-3-((2-(2,6-dioxopiperidin-3-yl)-1-oxoisoindolin-4-yl)amino)cyclohexyl)methyl)carbamate 8 (0.150 g, 0.313 mmol, 32.46% yield) as yellow solid. Analysis for tert-butyl (((1S,3S)-3-((2-(2,6-dioxopiperidin-3-yl)-1-oxoisoindolin-4-yl)amino)cyclohexyl)methyl)carbamate (7):

¹H-NMR (400 MHz, DMSO-d₆): δ 11.02 (s, 1H), 7.28 (t, J=7.60 Hz, 1H), 6.93 (d, J=7.60 Hz, 1H), 6.83 (q, J=8.00 Hz, 2H), 5.12 (q, J=5.20 Hz, 1H), 4.17 (q, J=17.20 Hz, 2H), 3.32 (t, J=11.20 Hz, 1H), 2.97-2.89 (m, 1H), 2.81 (d, J=6.00 Hz, 2H), 2.63 (d, J=16.80 Hz, 1H), 2.30 (q, J=4.40 Hz, 1H), 2.01 (q, J=7.60 Hz, 3H), 1.76 (d, J=13.20 Hz, 1H), 1.67 (t, J=12.40 Hz, 1H), 1.53 (d, J=8.00 Hz, 1H), 1.36 (s, 9H), 1.32 (s, 1H), 1.12 (s, 1H), 0.84-0.77 (m, 2H).

LCMS: 415.1 (M+H, t-Butyl cleaved mass). Method: Column: XBridge C8 (50×4.6 mm) 3.5 μm, Mobile phase A: 0.1% TFA in H₂O, B: 0.1% TFA in ACN, Flow Rate: 1.5 mL/min, Rt (min): 1.785, Area (%): 97.490.

HPLC: Method: A: 0.1% TFA in H₂O, B: ACN, Column: XBridge C8 (50×4.6 mm) 3.5 μm; Flow Rate: 2.0 mL/min; Rt (min): 3.674 & 3.734; Area (%): 47.297 & 50.285.

Analysis for tert-butyl (((1R,3S)-3-((2-(2,6-dioxopiperidin-3-yl)-1-oxoisoindolin-4-yl)amino)cyclohexyl)methyl)carbamate (8):

¹H-NMR (400 MHz, DMSO-d₆): δ 11.03 (s, 1H), 7.29-7.25 (m, 1H), 6.94 (d, J=7.60 Hz, 1H), 6.82 (d, J=7.60 Hz, 2H), 5.14 (q, J=5.20 Hz, 1H), 4.31-4.14 (m, 2H), 3.67 (d, J=2.00 Hz, 1H), 2.97-2.86 (m, 3H), 2.63 (d, J=17.20 Hz, 1H), 2.32 (t, J=13.20 Hz, 1H), 2.05-2.01 (m, 1H), 1.82 (s, 1H), 1.54-1.46 (m, 3H), 1.67-1.59 (m, 4H), 1.36 (d, J=2.80 Hz, 9H), 1.16 (d, J=5.20 Hz, 1H).

LCMS: 415.2 (M+H, t-butyl cleaved mass). Method: Column: XBridge C8 (50×4.6 mm) 3.5 μm, Mobile phase A: 0.1% TFA in H₂O, B: 0.1% TFA in ACN, Flow Rate: 1.5 mL/min, Rt (min): 1.865, Area (%): 97.869.

HPLC: Method: A: 0.1% TFA in H₂O, B: ACN, Column: XBridge C8 (50×4.6 mm) 3.5 μm; Flow Rate: 2.0 mL/min; Rt (min): 3.912 & 3.916; Area (%):44.673 & 53.434.

### Step-6: tert-butyl (((1R,3S)-3-((2-(2,6-dioxopiperidin-3-yl)-1-oxoisoindolin-4-yl)(pentyl)amino)cyclohexyl)methyl)carbamate (9)

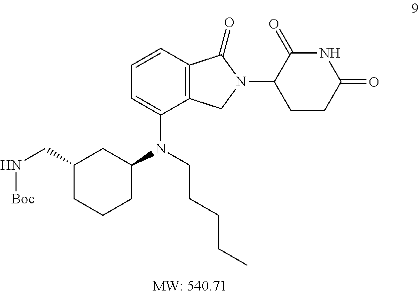 [SMILES: CCCCCN(c1cccc2c1CN(C1CCC(=O)NC1=O)C2=O)[C@H]1CCC[C@H](CNC(=O)OC(C)(C)C)C1]

To a mixture of tert-butyl (((1S,3S)-3-((2-(2,6-dioxopiperidin-3-yl)-1-oxoisoindolin-4-yl)amino)cyclohexyl)methyl)carbamate 7 (0.13 g, 0.276 mmol, 1.0 equiv), pentanal (0.238 g, 2.76 mmol, 10.0 equiv) in 1,2-dichloroethane (2.0 ml) and DMF (1.000 ml) was added TFA (0.064 ml, 0.829 mmol, 3.0 equiv) at 0° C. After 10 min STAB (0.293 g, 1.381 mmol, 5.0 equiv) was added in portions and the reaction mixture was stirred at room temperature for 2 h. Upon completion of reaction (as confirmed by LCMS), the reaction mixture was quenched with ice cold water (50 mL) and extracted with EtOAc (3×30 mL). The combined organic layer was washed with brine (1×50 mL), dried over Na₂SO₄, filtered and concentrated under reduced pressure to afford the crude product which was purified by preparative HPLC (Column: XBRIDGE C-18 19.1×150 MM, 500 ul, Mobile phase: A: 0.1% FA in water, B: MeCN, Flow rate: 15 mL/min) to get tert-butyl (((1S,3S)-3-((2-(2,6-dioxopiperidin-3-yl)-1-oxoisoindolin-4-yl)(pentyl)amino)cyclohexyl)methyl)carbamate 9 (0.130 g, 0.239 mmol, 86.6% yield) as off-white solid.

¹H-NMR (400 MHz, DMSO-d₆) δ 10.97 (s, 1H), 7.43-7.20 (m, 3H), 6.79 (t, J=6.80 Hz, 1H), 5.13-5.06 (m, 1H), 4.38-4.18 (m, 2H), 3.09 (s, 3H), 2.92-2.85 (m, 1H), 2.73 (t, J=2.40 Hz, 2H), 2.62 (s, 1H), 2.02 (t, J=7.60 Hz, 1H), 1.72 (d, J=13.20 Hz, 3H), 1.55 (d, J=15.20 Hz, 1H), 1.35 (s, 11H), 1.21 (s, 7H), 1.06 (q, J=14.80 Hz, 1H), 0.85-0.73 (m, 3H).

LCMS: 541.3 (M+H). Method: Column: XBridge C8 (50×4.6 mm) 3.5 μm, Mobile phase A: 0.1% TFA in H₂O, B: 0.1% TFA in ACN, Flow Rate: 1.5 mL/min, Rt (min): 1.942, Area (%): 98.172

HPLC: Method: A: 0.1% TFA in H₂O, B: ACN, Column: XBridge C8 (50×4.6 mm) 3.5 μm; Flow Rate: 2.0 mL/min; Rt (min): 3.824; Area (%): 99.601.

### Step-7: [3-(4-(((1S,3S)-3-(aminomethyl)cyclohexyl)(pentyl)amino)-1-oxoisoindolin-2-yl)piperidine-2,6-dione, HCl

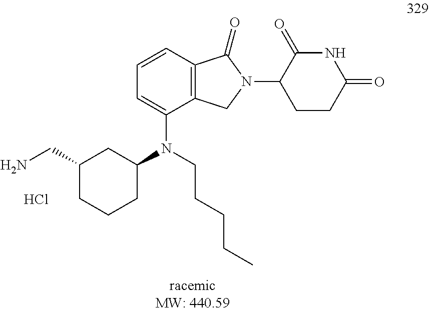 [SMILES: CCCCCN(c1cccc2c1CN(C1CCC(=O)NC1=O)C2=O)[C@H]1CCC[C@H](CN)C1 ; Cl]

To an ice cold solution of tert-butyl (((1S,3S)-3-((2-(2,6-dioxopiperidin-3-yl)-1-oxoisoindolin-4-yl)(pentyl)amino)cyclohexyl)methyl)carbamate 9 (0.130 g, 0.240 mmol, 1.0 equiv) in DCM (3.0 mL) was added hydrochloric acid (4.0 M sol in ethyl acetate, 1.202 ml, 4.81 mmol, 20.0 equiv) at 0° C. and the resultant reaction mixture was stirred 10 min. Then the reaction mixture was stirred at room temperature for 2 h. Upon completion of the reaction (as confirmed by LCMS & TLC analysis, 100% EtOAc, R_{f}˜0.1), the reaction mixture was concentrated under reduced pressure to get the crude product as pale yellow gummy solid which was washed with MTBE (2×10 mL). The residue was dried under reduced pressure and lyophilized to get [3-(4-(((1S,3S)-3-(aminomethyl)cyclohexyl)(pentyl)amino)-1-oxoisoindolin-2-yl)piperidine-2,6-dione, HCl 329 (0.095 g, 0.199 mmol, 83% yield)] as pale yellow solid.

¹H-NMR (400 MHz, DMSO-d₆): δ 11.00 (br s, 1H), 7.88 (br s, 3H), 7.43-7.29 (m, 3H), 5.13-5.10 (m, 1H), 4.27-4.26 (m, 1H), 3.59-3.11 (m, 2H), 2.96-2.88 (m, 1H), 2.67 (m, 4H), 2.03 (m, 1H), 1.91 (m, 1H), 1.85-1.75 (m, 2H), 1.67-1.65 (m, 3H), 1.43 (m, 1H), 1.41-1.21 (m, 9H), 0.85-0.79 (m, 4H).

LCMS: 441.4 (M+H), Method: Mobile phase: A: 0.1% TFA in H₂O, Mobile phase: B: 0.1% TFA in ACN, Column: XBridge C8 (50×4.6 mm) 3.5 μm, Flow Rate: 1.5 ml/min, Rt (min): 1.409, Area (%): 98.643.

HPLC: Method: Mobile phase: A: 0.1% TFA in water, Mobile phase: B: MeCN, Column: X Bridge C8 (50×4.6) mm, 3.5 μm, Flow: 2.0 mL/min, Rt (min): 2.418, Area (%): 99.826.

### Hydrochloride Salt Form of Compound 330

### 3-(4-(((1S,3R)-3-(aminomethyl)cyclohexyl)(pentyl)amino)-1-oxoisoindolin-2-yl)piperidine-2,6-dione, HCl

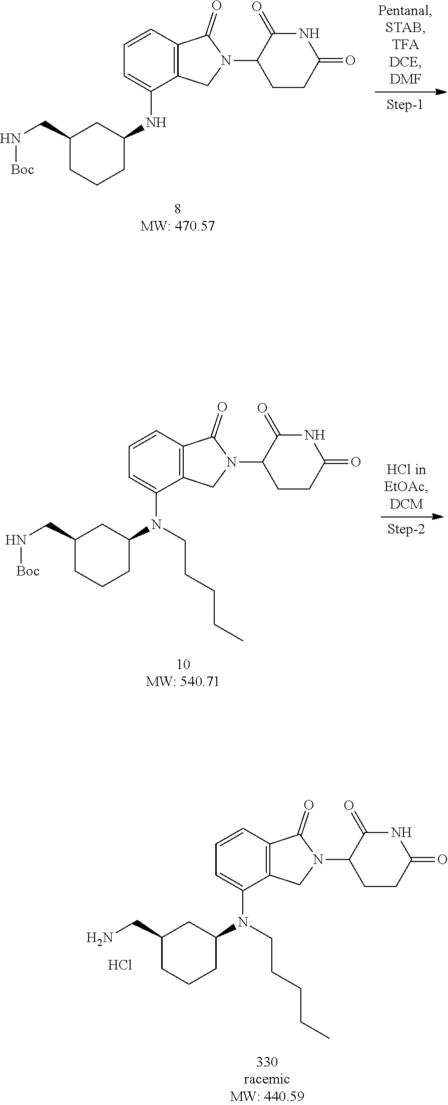 [SMILES: CC(C)(C)OC(=O)NC[C@@H]1CCC[C@H](Nc2cccc3c2CN(C2CCC(=O)NC2=O)C3=O)C1 ; CCCCCN(c1cccc2c1CN(C1CCC(=O)NC1=O)C2=O)[C@H]1CCC[C@@H](CNC(=O)OC(C)(C)C)C1 ; CCCCCN(c1cccc2c1CN(C1CCC(=O)NC1=O)C2=O)[C@H]1CCC[C@@H](CN)C1 ; Cl]

### Step-1: tert-butyl (((1R,3S)-3-((2-(2,6-dioxopiperidin-3-yl)-1-oxoisoindolin-4-yl)(pentyl)amino)cyclohexyl)methyl)carbamate (10)

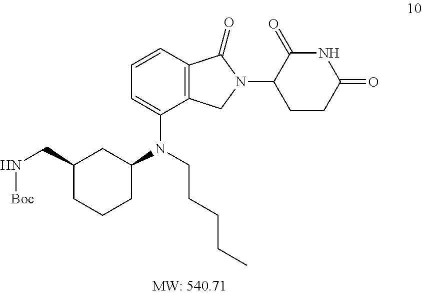 [SMILES: CCCCCN(c1cccc2c1CN(C1CCC(=O)NC1=O)C2=O)[C@H]1CCC[C@@H](CNC(=O)OC(C)(C)C)C1]

To a mixture of tert-butyl (((1R,3S)-3-((2-(2,6-dioxopiperidin-3-yl)-1-oxoisoindolin-4-yl)amino)cyclohexyl)methyl)carbamate 7 (0.150 g, 0.319 mmol, 1.0 equiv) and pentanal (0.275 g, 3.19 mmol, 10.0 equiv) in 1,2-dichloroethane (2.0 mL) and DMF (1.0 mL) was added TFA (0.074 ml, 0.956 mmol, 3.0 equiv) at 0° C. After 10 min STAB (0.338 g, 1.594 mmol, 5.0 equiv) was added in portions and the reaction mixture was stirred at room temperature for overnight. Upon completion of reaction (as confirmed by TLC analysis, 100% EtOAc, Rf˜0.5), the reaction mixture was quenched with ice cold water (50 mL) and extracted with EtOAc (3×30 mL). The combined organic layer was washed with brine (1×50 mL), dried over Na₂SO₄, filtered and concentrated under reduced pressure to afford the crude product which was purified by preparative HPLC (Column: XBRIDGE C-18 19.1 x150 MM, 500 ul, Mobile phase: A: 0.10% FA in water, B: MeCN, Flow rate: 15 mL/min) to get tert-butyl(((1R,3S)-3-((2-(2,6-dioxopiperidin-3-yl)-1-oxoisoindolin-4-yl)(pentyl)amino)cyclohexyl)methyl)carbamate 10 (0.160 g, 0.293 mmol, 82% yield) as off-white solid.

¹H-NMR (400 MHz, DMSO-d6): δ 10.97 (m, 1H), 7.44-7.26 (m, 3H), 6.83-6.76 (m, 1H), 5.16-5.08 (m, 1H), 4.41-4.21 (m, 2H), 3.40 (s, 1H), 3.06 (s, 2H), 2.92 (d, J=10.00 Hz, 2H), 2.85 (d, J=8.40 Hz, 2H), 2.58 (m, 2H), 2.01 (t, J=6.40 Hz, 1H), 1.87 (s, 1H), 1.67 (m, 1H), 1.49 (m, 3H), 1.33 (d, J=8.40 Hz, 12H), 1.26-1.11 (m, 8H), 0.79 (q, J=8.80 Hz, 3H).

LCMS: 541.4 (M+H), Method: Mobile phase: A: 0.1% TFA in H₂O, Mobile phase: B: 0.1% TFA in ACN, Column: XBridge C8 (50×4.6 mm) 3.5 μm, Flow Rate: 1.5 ml/min, Rt (min): 1.942, Area (%): 98.817.

HPLC: Method: Mobile phase: A: 0.1% TFA in water, Mobile phase: B: MeCN, Column: X Bridge C8 (50×4.6) mm, 3.5 μm, Flow: 2.0 mL/min, Rt (min): 3.899, Area (%): 99.085.

### Step-2: 3-(4-(((1S,3R)-3-(aminomethyl)cyclohexyl)(pentyl)amino)-1-oxoisoindolin-2-yl)piperidine-2,6-dione, HCl

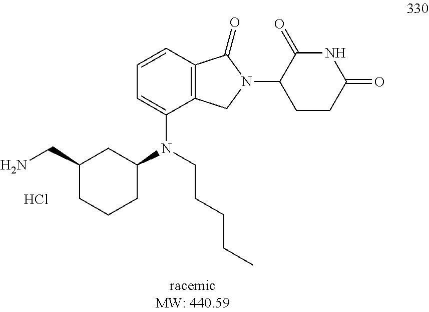 [SMILES: CCCCCN(c1cccc2c1CN(C1CCC(=O)NC1=O)C2=O)[C@H]1CCC[C@@H](CN)C1 ; Cl]

To an ice cold solution of tert-butyl (((1R,3S)-3-((2-(2,6-dioxopiperidin-3-yl)-1-oxoisoindolin-4-yl)(pentyl)amino)cyclohexyl)methyl)carbamate 10 (0.240 g, 0.444 mmol, 1.0 equiv) in dichloromethane (3.0 mL) was added hydrochloric acid (4.0M solution in EtOAc, 2.219 mL, 8.88 mmol, 20.0 equiv) at 0° C. and the resultant reaction mixture was stirred for 10 min. Then the reaction mixture was allowed to warm to room temperature and stirred for 2 h. Upon completion of the reaction (as confirmed by TLC analysis, 100% EtOAc, Rf˜0.0), the reaction mixture was concentrated under reduced pressure to get the crude product as off-white solid which was washed with MTBE (2×5 mL), dried under reduced pressure and lyophilized to afford 3-(4-(((1S,3R)-3-(aminomethyl)cyclohexyl)(pentyl)amino)-1-oxoisoindolin-2-yl)piperidine-2,6-dione, HCl 330 (0.201 g, 0.420 mmol, 95% yield)] as pale yellow solid.

¹H-NMR (400 MHz, DMSO-d₆): δ 11.00 (br s, 1H), 8.22-7.93 (m, 4H), 7.43-7.32 (m, 2H), 5.18-5.11 (m, 1H), 4.41-4.30 (m, 2H), 3.38-3.37 (m, 1H), 3.17-2.83 (m, 5H), 2.68-2.61 (m, 2H), 2.13-1.92 (m, 2H), 1.78-1.44 (m, 8H), 1.19 (m, 6H), 0.78 (t, J=6.80 Hz, 3H).

LCMS: 441.3 (M+H), Method: Mobile phase: A: 0.1% TFA in H₂O, Mobile phase: B: ACN, Column: XBridge C8 (50×4.6 mm) 3.5 μm, Flow Rate: 2.0 ml/min, Rt (min): 1.144, Area (%): 99.905.

HPLC: Method: Mobile phase: A: 0.1% TFA in water, Mobile phase: B: MeCN, Column: X Bridge C8 (50×4.6) mm, 3.5 μm, Flow:2.0 mL/min, Rt (min): 2.553, Area (%): 99.796.

### Compound 331

The compound having the following structure is prepared in analogy from the preparation procedure described for the above-mentioned compounds.

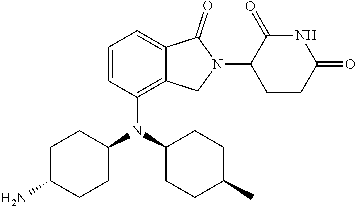 [SMILES: C[C@H]1CC[C@@H](N(c2cccc3c2CN(C2CCC(=O)NC2=O)C3=O)[C@H]2CC[C@H](N)CC2)CC1]

### Compound 332

The compound having the following structure is prepared in analogy from the preparation procedure described for the above-mentioned compounds.

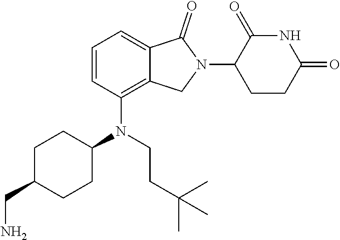 [SMILES: CC(C)(C)CCN(c1cccc2c1CN(C1CCC(=O)NC1=O)C2=O)[C@H]1CC[C@@H](CN)CC1]

### Compound 333

The compound having the following structure is prepared in analogy from the preparation procedure described for the above-mentioned compounds.

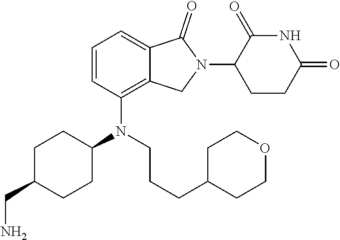 [SMILES: NC[C@H]1CC[C@@H](N(CCCC2CCOCC2)c2cccc3c2CN(C2CCC(=O)NC2=O)C3=O)CC1]

### Compound 334

The compound having the following structure is prepared in analogy from the preparation procedure described for the above-mentioned compounds.

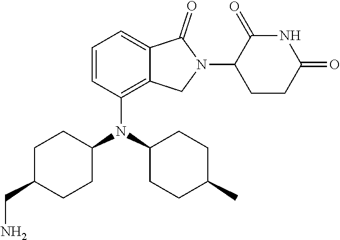 [SMILES: C[C@H]1CC[C@@H](N(c2cccc3c2CN(C2CCC(=O)NC2=O)C3=O)[C@H]2CC[C@@H](CN)CC2)CC1]

### Hydrochloride Salt Form of Compound 340

### 3-(4-(((1r,4r)-4-aminocyclohexyl)(3-(tetrahydro-2H-pyran-3-yl)propyl)amino)-1-oxoisoindolin-2-yl)piperidine-2,6-dione, HCl

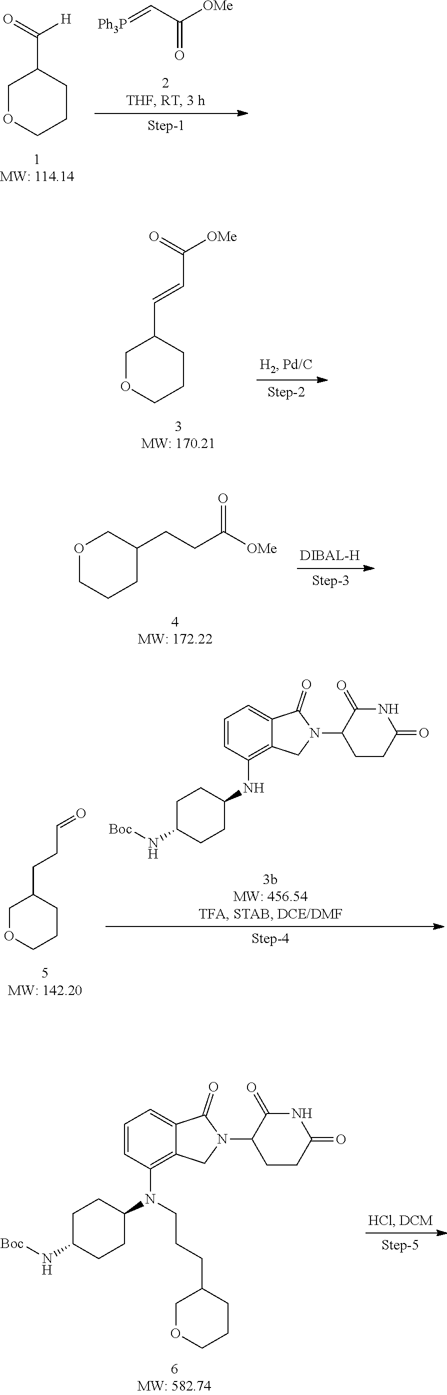

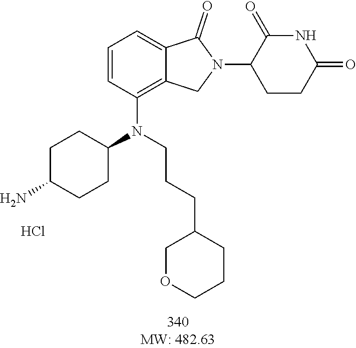 [SMILES: N[C@H]1CC[C@H](N(CCCC2CCCOC2)c2cccc3c2CN(C2CCC(=O)NC2=O)C3=O)CC1 ; Cl]

### Step-1: (E)-3-(tetrahydro-2H-pyran-3-yl)acrylate (3)

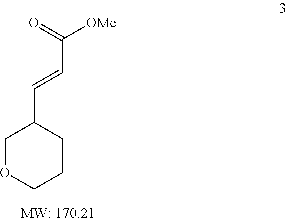 [SMILES: COC(=O)/C=C/C1CCCOC1]

To a solution of tetrahydro-2H-pyran-3-carbaldehyde 1 (1 g, 8.76 mmol, 1 equiv) in THF (10 ml) was added methyl 2-(triphenyl-5-phosphanylidene)acetate 2 (3.22 g, 9.64 mmol, 1 equiv) at room temperature and the reaction mixture was stirred at room temperature for 16 h. Upon completion of reaction (as confirmed by TLC analysis, 20% EtOAc in pet ether, Rf: 0.7), the reaction mixture was concentrated under reduced pressure to give a pink solid which was taken in hexane (50 ml), stirred for 1 h and filtered. The filtrate was concentrated under vacuum to give the crude which was purified by Isolera (column size: Biotage R snap cartridge, KP-Sil, 25 g, 230-400 silica gel) using 10-15% of ethyl acetate in pet ether. The pure fractions were concentrated under vacuum to get methyl (E)-3-(tetrahydro-2H-pyran-3-yl)acrylate 3 (1.2 g, 7.03 mmol, 80% yield) as colorless oil.

¹H-NMR (400 MHz, DMSO-d6): δ 6.78 (q, J=7.20 Hz, 1H), 5.91 (dd, J=1.20, 15.60 Hz, 1H), 3.80-3.74 (m, 2H), 3.65 (d, J=2.80 Hz, 3H), 3.35-3.29 (m, 1H), 3.19 (q, J=9.60 Hz, 1H), 2.43 (d, J=6.00 Hz, 1H), 1.83 (q, J=2.00 Hz, 1H), 1.58-1.50 (m, 2H), 1.47-1.41 (m, 1H).

LCMS: 171.3 (M+H), Rt (min): 2.329, Area (%): 99.739.

### Step-2: methyl 3-(tetrahydro-2H-pyran-3-yl)propanoate (4)

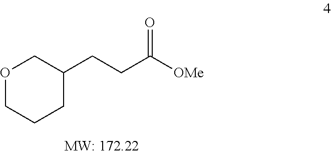 [SMILES: COC(=O)CCC1CCCOC1]

To a solution of methyl (E)-3-(tetrahydro-2H-pyran-3-yl)acrylate 3 (1.2 g, 7.05 mmol, 1 equiv) in MeOH (20 ml) was added Pd—C (10% wt/wt, 0.150 g, 0.141 mmol, 0.02 equiv) and the reaction mixture was stirred under hydrogen bladder at room temperature for 16 h. Upon completion of reaction (as confirmed by TLC analysis, 20% EtOAc in pet ether, Rf: 0.4), the reaction mixture was filtered through a Celite bed, washed with MeOH (2×10 mL) and the filtrate was concentrated under vacuum to get the crude of methyl 3-(tetrahydro-2H-pyran-3-yl)propanoate 4 (1 g, 5.81 mmol, 82% yield) pale yellow liquid.

¹H-NMR (400 MHz, DMSO-d6): δ 3.75-3.72 (m, 2H), 3.58 (s, 3H), 3.27-3.21 (m, 1H), 2.95 (q, J=9.60 Hz, 1H), 2.33-2.29 (m, 2H), 1.52-1.50 (m, 1H), 1.47-1.43 (m, 1H), 1.42-1.32 (m, 4H), 1.02-1.00 (m, 1H).

### Step-3: 3-(tetrahydro-2H-pyran-3-yl)propanal (5)

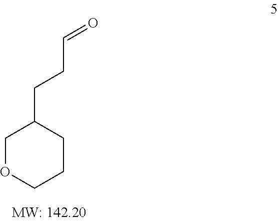

To a solution of methyl 3-(tetrahydro-2H-pyran-3-yl)propanoate 4 (1 g, 5.81 mmol, 1 equiv) in DCM (10.0 ml) was added DIBAL-H (1.2 M soln., in toluene, 5.32 ml, 6.39 mmol, 1.1 equiv) in drops at −78° C. The resultant solution was allowed to warm to room temperature and stirred for 1 h at room temperature. Upon completion of reaction (as confirmed by TLC analysis, 25% EtOAc in pet ether, Rf: 0.2), the reaction mixture was quenched with MeOH (5 mL) at 0° C., filtered through Celite bed and washed with DCM (1×25 mL). The filtrate was concentrated under vacuum to afford the crude of 3-(tetrahydro-2H-pyran-3-yl)propanal 5 (820 mg, 5.77 mmol, 99% yield) as a colorless liquid.

GCMS:142.10; Rt (min): 6.851, Area (%): 48.49.

### Step-4: tert-butyl ((1r,4r)-4-((2-(2,6-dioxopiperidin-3-yl)-1-oxoisoindolin-4-yl)(3-(tetrahydro-2H-pyran-3-yl)propyl)amino)cyclohexyl)carbamate (6)

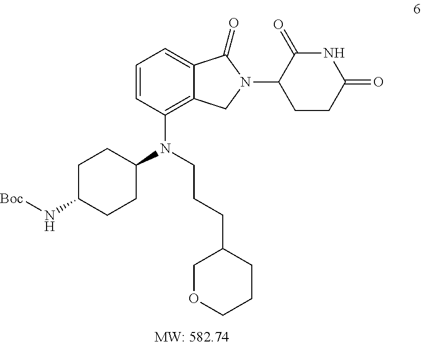 [SMILES: CC(C)(C)OC(=O)N[C@H]1CC[C@H](N(CCCC2CCCOC2)c2cccc3c2CN(C2CCC(=O)NC2=O)C3=O)CC1]

To a mixture of tert-butyl ((1r,4r)-4-((2-(2,6-dioxopiperidin-3-yl)-1-oxoisoindolin-4-yl)amino)cyclohexyl)carbamate 88-3b (200 mg, 0.438 mmol, 1 equiv) and 3-(tetrahydro-2H-pyran-3-yl)propanal 5 (809.67 mg, 5.694 mmol, 13 equiv) in DCE (2 mL) and DMF (0.5 ml) was added TFA (0.068 ml, 0.876 mmol, 4 equiv) and STAB (371 mg, 1.752 mmol, 8 equiv) at 0° C. and allowed to stir at room temperature for 24 h. Upon completion of reaction (as confirmed by LCMS) the reaction mixture was quenched with ice water (20 ml) and extracted with DCM (2×10 ml). The combined organic layer was concentrated under vacuum to give crude which was purified by preparative HPLC (Column: XBridge C18-19.1×150 mm; Method: 0.1% FA in ACN, flow rate: 20 ml/min). The pure fractions were lyophilised to get tert-butyl ((1r,4r)-4-((2-(2,6-dioxopiperidin-3-yl)-1-oxoisoindolin-4-yl)(3-(tetrahydro-2H-pyran-3-yl)propyl)amino)cyclohexyl)carbamate 6 (36 mg, 0.062 mmol, 17.94% yield) as yellow solid.

¹H-NMR (400 MHz, DMSO-d6): δ 10.97 (s, 1H), 7.42 (t, J=7.60 Hz, 1H), 7.31 (d, J=7.60 Hz, 1H), 7.26 (d, J=8.00 Hz, 1H), 6.68 (d, J=7.60 Hz, 1H), 5.16 (s, 1H), 4.30 (m, 2H), 3.98 (d, J=6.80 Hz, 1H), 3.73 (d, J=11.60 Hz, 3H), 3.58 (d, J=20.00 Hz, 1H), 3.32-3.25 (m, 2H), 3.23 (t, J=2.80 Hz, 3H), 3.08-2.93 (m, 2H), 2.92-2.61 (m, 2H), 2.57-2.50 (m, 2H), 2.30 (s, 3H), 1.80 (s, 1H), 1.56 (d, J=4.80 Hz, 1H), 1.48-1.45 (m, 2H), 1.52-1.49 (m, 2H), 1.52 (d, J=4.00 Hz, 3H), 1.37 (s, 3H), 1.18 (q, J=3.20 Hz, 5H), 1.11 (t, J=2.00 Hz, 1H), 1.08 (t, J=3.60 Hz, 1H).

LCMS: 583.3 (M+H), Rt (min): 2.424, Area (%): 98.902. HPLC: RT (min): 3.484, Area (%): 99.643.

### Step-5: 3-(4-(((1r,4r)-4-aminocyclohexyl)(3-(tetrahydro-2H-pyran-3-yl)propyl)amino)-1-oxoisoindolin-2-yl)piperidine-2,6-dione, HCl (340)

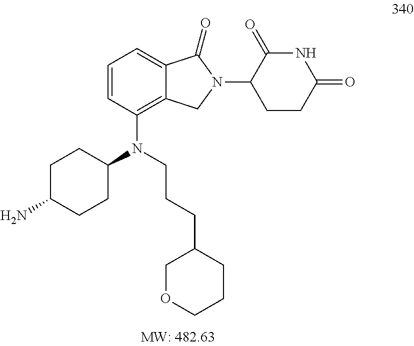 [SMILES: N[C@H]1CC[C@H](N(CCCC2CCCOC2)c2cccc3c2CN(C2CCC(=O)NC2=O)C3=O)CC1]

To an ice cold solution of tert-butyl ((1r,4r)-4-((2-(2,6-dioxopiperidin-3-yl)-1-oxoisoindolin-4-yl)(3-(tetrahydro-2H-pyran-3-yl)propyl)amino)cyclohexyl)carbamate 6 (35 mg, 0.060 mmol, 1 equiv) in DCM (1 ml) was added hydrogen chloride (4 M in ethyl acetate, 0.150 ml, 0.601 mmol, 10 equiv) and was stirred for 1 h at room temperature. Upon completion of reaction (as confirmed by LCMS), the reaction mixture was concentrated and the solid was washed with MTBE (10 ml), dried under vacuum and lyophilized to get 3-(4-(((1r,4r)-4-aminocyclohexyl)(3-(tetrahydro-2H-pyran-3-yl)propyl)amino)-1-oxoisoindolin-2-yl)piperidine-2,6-dione, HCl 340 (23 mg, 0.044 mmol, 73.1% yield) as yellow solid.

¹H-NMR (400 MHz, DMSO-d6): δ 10.99 (s, 1H), 7.83 (s, 3H), 7.44 (t, J=8.40 Hz, 1H), 7.31 (q, J=8.00 Hz, 2H), 5.12 (t, J=5.60 Hz, 1H), 4.25 (s, 1H), 4.31 (s, 1H), 3.66 (d, J=19.60 Hz, 1H), 3.62 (d, J=5.60 Hz, 1H), 3.60 (s, 1H), 3.16 (m, 5H), 2.97-2.93 (m, 1H), 2.68-2.67 (m, 1H), 2.00-1.94 (m, 3H), 1.79 (d, J=11.60 Hz, 2H), 1.59-1.34 (m, 9H), 1.24-1.11 (m, 2H), 1.09-0.98 (m, 3H).

LCMS: 483.3 (M+H), Method: Mobile phase: A: 0.1% TFA in H₂O, Mobile phase: B: ACN, Column: XBridge C8 (50×4.6 mm) 3.5 μm, Flow Rate: 2.0 ml/min, Rt (min): 1.013, Area %: 97.306.

HPLC: Method: Mobile phase: A: 0.1% TFA in water, Mobile phase: B: ACN, Column: X-Bridge C8(50×4.6) mm, 3.5 μm Flow: 2.0 mL/min, Rt (min): 2.247, Area %: 99.135.

### Hydrochloride Salt Form of Compound 341

### 3-(4-(((1r,4r)-4-aminocyclohexyl)(3-(tetrahydro-2H-pyran-2-yl)propyl)amino)-1-oxoisoindolin-2-yl)piperidine-2,6-dione, HCl

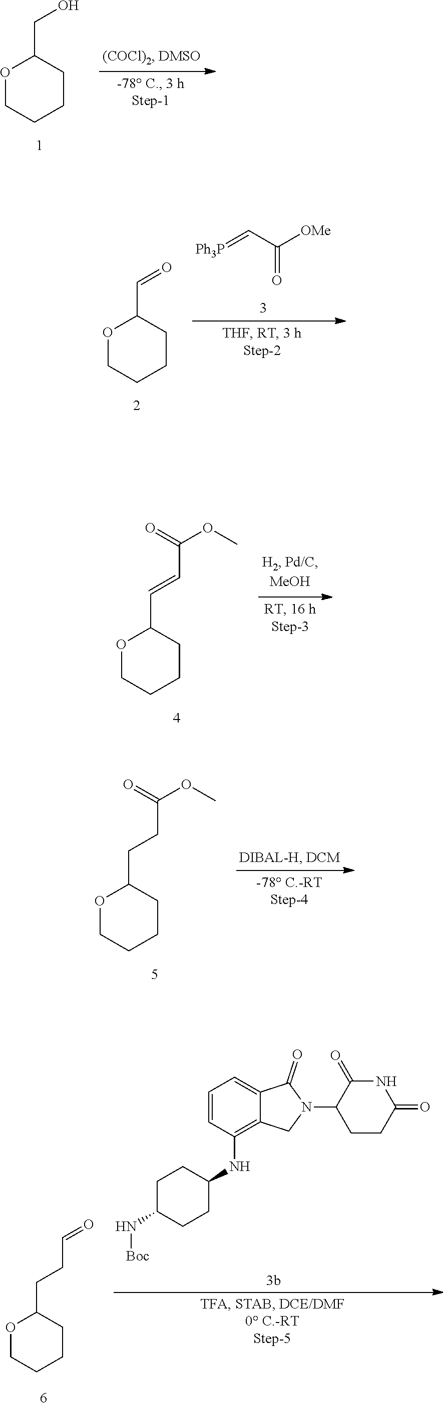

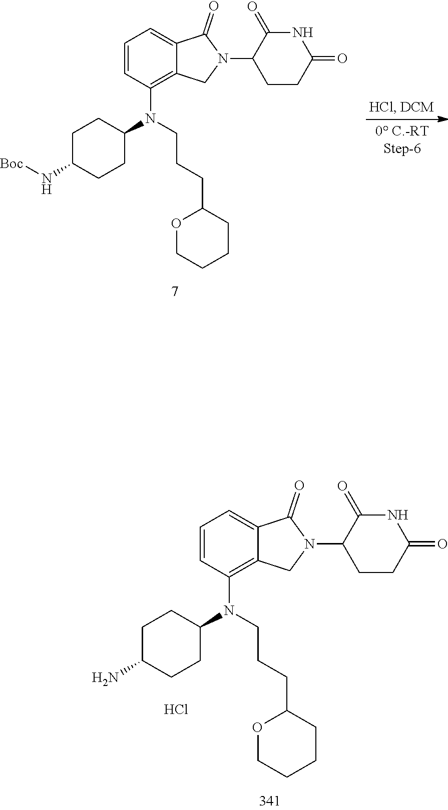 [SMILES: CC(C)(C)OC(=O)N[C@H]1CC[C@H](N(CCCC2CCCCO2)c2cccc3c2CN(C2CCC(=O)NC2=O)C3=O)CC1 ; N[C@H]1CC[C@H](N(CCCC2CCCCO2)c2cccc3c2CN(C2CCC(=O)NC2=O)C3=O)CC1 ; Cl]

### Step-1: tetrahydrofuran-2-carbaldehyde

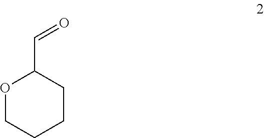

To a stirred solution of oxalyl chloride (2.215 ml, 25.8 mmol, 3 equiv) in DCM (20 mL) were added (methylsulfinyl)methane (3.06 ml, 43.0 mmol, 6 equiv) in DCM (5 mL) at −78° C. and was allowed to stirred for 20 min. To this mixture, a solution of (tetrahydro-2H-pyran-2-yl) methanol 1 (1 g, 8.61 mmol, 1 equiv) in DCM (5 mL) was added and stirred for 1 h. Then triethylamine (10.83 ml, 77 mmol, 9 equiv) was added and the reaction mixture was stirred for 15 minutes at −78° C. Upon completion of the reaction (as confirmed by TLC analysis, 40% EtOAc in pet ether, R_{f} of the product˜0.6), the reaction mixture was quenched with a saturated solution of citric acid (30 mL) and extracted with DCM (3×50 mL). The organic layer was dried over sodium sulphate filtered and concentrated under reduced pressure to afford crude tetrahydro-2H-pyran-2-carbaldehyde 2 (1.52 g, 13.14 mmol, 134% yield) as brown liquid. The crude compound as such used for next step.

### Step-2: methyl (E)-3-(tetrahydrofuran-2-yl) acrylate

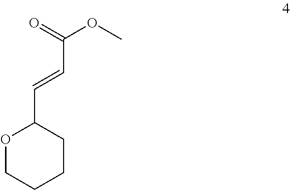

To a stirred solution of tetrahydro-2H-pyran-2-carbaldehyde 2 (1.52 g, 13.14 mmol, 1 equiv) in dry acetonitrile (30 mL) was added methyl 2-(triphenyl-15-phosphanylidene) acetate 3 (4.83 g, 14.46 mmol, 1.1 equiv) at room temperature under nitrogen atmosphere. The mixture was stirred for overnight at room temperature. Upon completion of the reaction (as confirmed by TLC analysis, 40% EtOAc in pet ether, R_{f} of the product˜0.47), the solvent was evaporated under reduced pressure, added 100 mL pet ether, stirred for 10 minutes and filtered through sintered funnel. The filtrate was concentrated under reduced pressure to afford the crude product as colorless liquid. The crude product was purified by column chromatography (isolera, silica mesh size 230-400, flow rate 25 mL/min). The column was eluted with 8-11% ethyl acetate in pet ether to afford methyl (E)-3-(tetrahydro-2H-pyran-2-yl)acrylate 4 (600 mg, 3.50 mmol, 26.6% yield) as colorless liquid.

LCMS: 171.1 [(M+H)], Method: Mobile phase: A: 0.1% FA in H₂O, Mobile phase: B: 0.1% FA in ACN, Column: Atlantis dC18 (50×4.6 mm) 5 μm, Flow Rate: 1.5 ml/min Rt (min): 2.119, Area %: 99.344.

1H-NMR (400 MHz, DMSO-d6): δ 6.84 (dd, J=4.00, 16.00 Hz, 1H), 5.92 (dd, J=2.00, 15.80 Hz, 1H), 3.92-3.99 (m, 2H), 3.66 (s, 3H), 3.46-3.33 (m, 1H), 1.78-1.81 (m, 2H), 1.44-1.54 (m, 4H), 1.22-1.16 (m, 1H).

### Step-3: methyl 3-(tetrahydrofuran-2-yl)propanoate

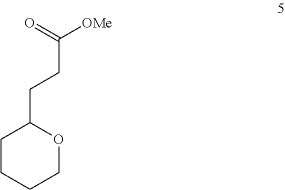 [SMILES: COC(=O)CCC1CCCCO1]

To a solution of methyl (E)-3-(tetrahydro-2H-pyran-2-yl) acrylate 4 (0.65 g, 3.82 mmol) in MeOH (15 ml) was added Pd—C (10% wt/wt) (0.092 g, 0.076 mmol) and stirred under hydrogen atmosphere (1 atm) at room temperature for overnight. Upon completion of the reaction (TLC analysis, 20% EtOAc in pet ether, R_{f}˜0.3), the reaction mixture was filtered through Cellite bed and washed with MeOH (2×80 mL) and concentrated under reduced pressure to afford crude product methyl 3-(tetrahydro-2H-pyran-2-yl)propanoate 5 (0.45 g, 2.102 mmol, 55.0% yield) as colorless liquid.

1H-NMR (400 MHz, CDCl₃): δ 3.85 (d, J=2.00 Hz, 1H), 3.58 (s, 3H), 3.33-3.26 (m, 2H), 3.26-3.16 (m, 1H), 2.37-2.29 (m, 2H), 1.75-1.73 (m, 1H), 1.58-1.64 (m, 2H), 1.55-1.52 (m, 1H), 1.45-1.40 (m, 2H), 1.37-1.40 (m, 1H)

### Step-4: 3-(tetrahydrofuran-2-yl)propanal

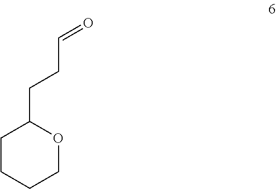

To a stirred solution of methyl 3-(tetrahydro-2H-pyran-2-yl)propanoate 5 (0.46 g, 2.67 mmol, 1.0 equiv) in dry dichloromethane (15.00 mL) at −78° C. under nitrogen atmosphere was added dropwise DIBAL-H (1.2 M sol in toluene, 2.448 ml, 2.94 mmol, 1.1 equiv) under nitrogen atmosphere. The resultant solution was allowed to warm to room temperature and stirred for 20 min. Upon completion of the reaction (as confirmed by TLC analysis, 40% EtOAc in pet ether, R_{f}˜0.3), the reaction mixture was quenched with methanol (2 mL) and saturated aqueous sodium potassium tartrate solution (10 mL) at 0° C., stirred for 10 minutes, filtered through Celite bed and washed with DCM (1×25 mL). The organic layer was separated and the aqueous layer was extracted with DCM (2×50 mL). The combined organic layer was washed with brine (50 mL), dried over Na₂SO₄, filtered and concentrated under reduced pressure to afford the crude 3-(tetrahydro-2H-pyran-2-yl) propanal 6 (0.39 g, 2.74 mmol, 103% yield) as pale yellow liquid. The crude compound was used for next step without further purification.

### Step-5: tert-butyl ((1r,4r)-4-((2-(2,6-dioxopiperidin-3-yl)-1-oxoisoindolin-4-yl)(3-(tetrahydro-2H-pyran-4-yl)propyl)amino)cyclohexyl)carbamate

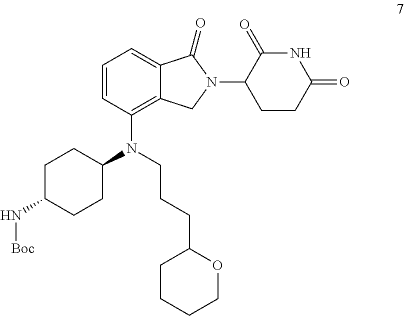 [SMILES: CC(C)(C)OC(=O)N[C@H]1CC[C@H](N(CCCC2CCCCO2)c2cccc3c2CN(C2CCC(=O)NC2=O)C3=O)CC1]

To a stirred solution of tert-butyl ((1r,4r)-4-((2-(2,6-dioxopiperidin-3-yl)-1-oxoisoindolin-4-yl)amino)cyclohexyl)carbamate 3b (0.12 g, 0.263 mmol, 1.0 equiv) and 3-(tetrahydro-2H-pyran-2-yl)propanal 6 (0.374 g, 2.63 mmol, 10 equiv) in a mixture of DCE (4 mL) and DMF (2 mL) were added trifluoroacetic acid (0.060 ml, 0.789 mmol 3.0 equiv) and sodium triacetoxyborohydride (0.279 g, 1.314 mmol 5.0 equiv) at 0° C. The resulting reaction mixture was stirred at room temperature for overnight. Upon completion of the reaction (as confirmed by LCMS analysis), the reaction mixture was poured into ice cold water (50 mL) and extracted with DCM (3×30 mL). The combined organic layer was washed with brine (100 mL), dried over anhydrous sodium sulphate, filtered and concentrated to get the crude. The crude compound was purified by preparative HPLC (Column: Select C18 250, Mobile phase: 0.1% FA in ACN, flow rate: 15 mL/min) and lyophilized to afford tert-butyl ((1r,4r)-4-((2-(2,6-dioxopiperidin-3-yl)-1-oxoisoindolin-4-yl)(3-(tetrahydro-2H-pyran-2-yl)propyl)amino)cyclohexyl)carbamate 7 (55 mg, 0.094 mmol, 36.7% yield) as white solid.

¹H-NMR (400 MHz, DMSO-d₆): δ 10.97 (s, 1H), 7.41 (t, J=7.60 Hz, 1H), 7.30-7.24 (m, 2H), 6.68 (d, J=8.00 Hz, 1H), 5.10 (dd, J=4.80, 13.20 Hz, 1H), 4.34-4.21 (m, 2H), 3.79-3.76 (m, 1H), 3.32-3.26 (m, 2H), 3.23-3.17 (m, 4H), 3.09-2.88 (m, 1H), 2.62-2.50 (m, 2H), 2.02-1.99 (m, 1H), 1.81-1.71 (m, 6H), 1.53-1.48 (m, 4H), 1.34-1.46 (m, 9H), 1.34-1.32 (m, 4H), 1.28-1.23 (m, 4H), 1.20-1.06 (m, 1H).

LCMS: 583.3 [(M+H)], Method: Mobile phase: A: 0.1% FA in H₂O, Mobile phase: B: 0.1% TFA in ACN, Column: Atlantis dC18 (50×4.6 mm) 5 μm, Flow Rate: 1.5 ml/min Rt (min): 2.458, Area %: 96.846.

HPLC: Method: Mobile phase: A:0.1% FA in water, Mobile phase: B: MeCN, Column: Xbridge C8(50×4.6) mm, 3.5 μm, Flow: 2.0 mL/min, Rt (min): 4.454 Area %: 99.712.

### Step-6: 3-(4-(((1r,4r)-4-aminocyclohexyl)(3-(tetrahydrofuran-2-yl)propyl)amino)-1-oxoisoindolin-2-yl)piperidine-2,6-dione, HCl

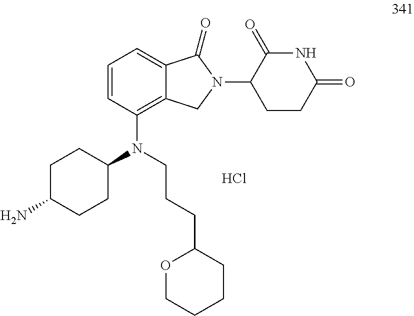 [SMILES: N[C@H]1CC[C@H](N(CCCC2CCCCO2)c2cccc3c2CN(C2CCC(=O)NC2=O)C3=O)CC1 ; Cl]

To a stirred solution of tert-butyl ((1r,4r)-4-((2-(2,6-dioxopiperidin-3-yl)-1-oxoisoindolin-4-yl)(3-(tetrahydro-2H-pyran-2-yl)propyl)amino)cyclohexyl)carbamate 7 (0.055 g, 0.094 mmol) in DCM (5 ml) was added hydrochloric acid (4 M sol in ethyl acetate, 2.231 ml, 8.92 mmol, 20 equiv) at 0° C. The resultant reaction mixture was allowed to room temperature and stirred for 2 h. Upon completion of the reaction (as confirmed by TLC analysis, 100% EtOAc, Rf of the product˜0.1), the reaction mixture was concentrated under reduced pressure to get the crude product as pale yellow gummy solid. The obtained solid was washed with MTBE (2×10 mL), dried under reduced pressure and lyophilized to afford 3-(4-(((1r,4r)-4-aminocyclohexyl)(3-(tetrahydro-2H-pyran-2-yl)propyl)amino)-1-oxoisoindolin-2-yl)piperidine-2,6-dione, HCl 341 (30.5 mg, 0.059 mmol, 62.3% yield) as white solid.

1H-NMR (400 MHz, DMSO-d₆): δ 11.00 (s, 1H), 7.88-7.95 (m, 3H), 7.43-7.31 (m, 3H), 5.12 (s, 1H), 3.83-3.76 (m, 2H), 3.09-3.08 (m, 3H), 2.97-2.92 (m, 2H), 2.55-2.68 (m, 2H), 1.98-1.95 (m, 3H), 1.77-1.68 (m, 3H), 1.60-1.56 (m, 2H), 1.45-1.30 (m, 11H), 1.29-1.27 (m, 1H).

LCMS: 483.2 (M+H), Method: Mobile phase: A: 0.1% FA in H₂O, Mobile phase: B: ACN, Column: Atlantis dC18 (50×4.6 mm) 5 μm, Flow Rate:1.5 ml/min Rt (min): 1.680, Area %: 97.045.

HPLC: Method: Mobile phase: A: 0.1% FA in water, Mobile phase: B: MeCN, Column: X Bridge C8 (50×4.6) mm, 3.5 μm, Flow:2.0 mL/min, Rt (min): 2.302, Area %: 96.067.

### Hydrochloride Salt Form of Compound 344

### 3-(4-(((1r,4r)-4-aminocyclohexyl)(3-(tetrahydro-4H-pyran-4-ylidene)propyl)amino)-1-oxoisoindolin-2-yl)piperidine-2,6-dione. Formic Acid

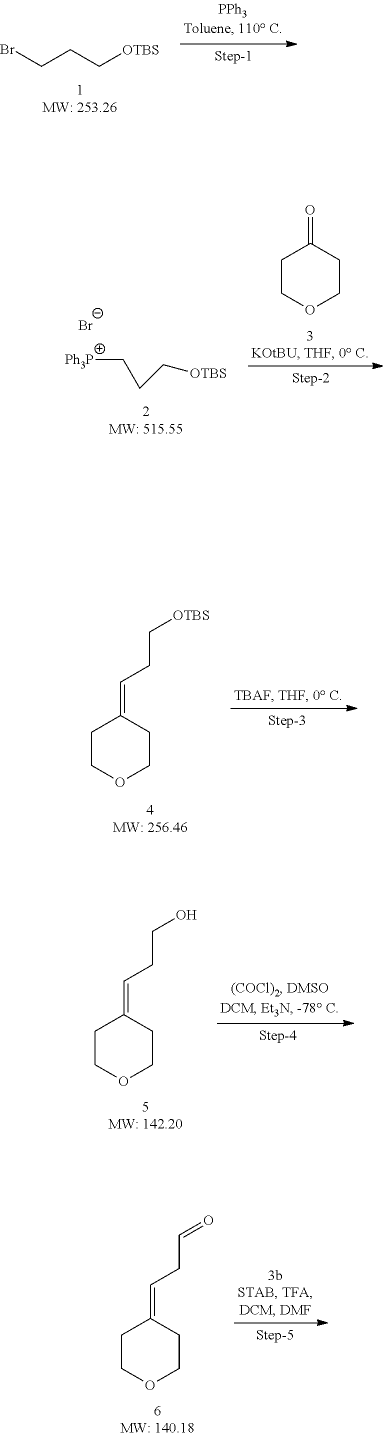

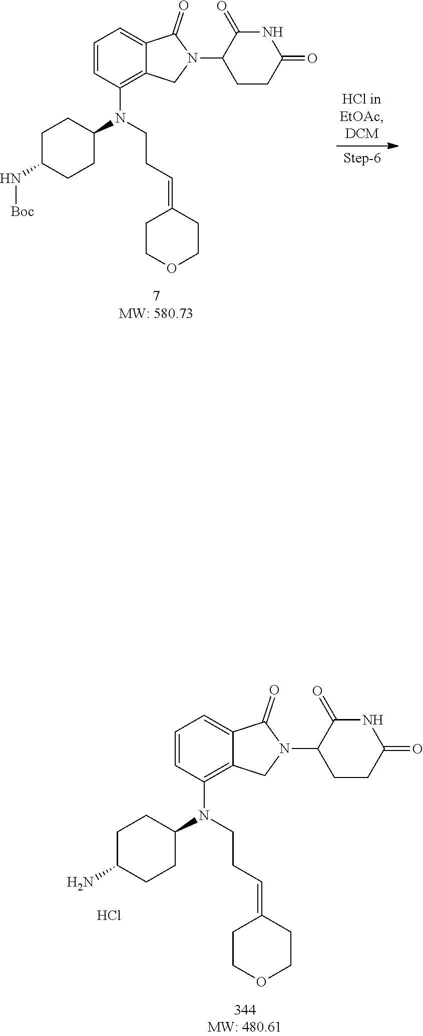 [SMILES: CC(C)(C)OC(=O)N[C@H]1CC[C@H](N(CCC=C2CCOCC2)c2cccc3c2CN(C2CCC(=O)NC2=O)C3=O)CC1 ; N[C@H]1CC[C@H](N(CCC=C2CCOCC2)c2cccc3c2CN(C2CCC(=O)NC2=O)C3=O)CC1 ; Cl]

### Step-1: (3-((tert-butyldimethylsilyl)oxy)propyl)triphenylphosphonium, Bromide (2)

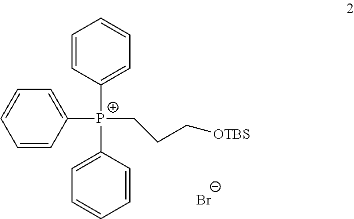 [SMILES: CC(C)(C)[Si](C)(C)OCCC[P+](c1ccccc1)(c1ccccc1)c1ccccc1 ; [Br-]]

To a solution of triphenylphosphine (15.54 g, 59.2 mmol, 1.5 equiv) in toluene (80 ml) was added (3-bromopropoxy)(tert-butyl)dimethylsilane 1 (10 g, 39.5 mmol, 1 equiv) and was heated to 110° C. for 16 h. Upon completion of the reaction (as confirmed by TLC analysis, 10% MeOH in DCM, Rf: 0.2), the reaction mixture was allowed to cool to room temperature and diluted with 50% MTBE/Hexane (100 mL). The precipitated solid was collected by filtration and dried to get (3-((tert-butyldimethylsilyl)oxy)propyl)triphenylphosphonium, Bromide 2 (12.2 g, 14.75 mmol, 37.4% yield) as white solid.

¹H-NMR (400 MHz, DMSO-d6): δ 7.94-7.89 (m, 3H), 7.83-7.75 (m, 12H), 3.70 (t, J=6.00 Hz, 2H), 3.59-3.51 (m, 2H), 1.76-1.67 (m, 2H), 0.85 (d, J=2.80 Hz, 9H), 0.02 (s, 6H).

LCMS: 435.1 (M-Br), Rt (min): 2.717, Area (%): 62.350.

### Step-2: tert-butyldimethyl(3-(tetrahydro-4H-pyran-4-ylidene)propoxy)silane (4)

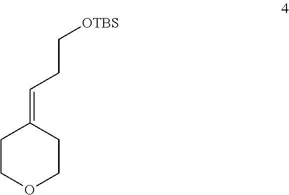 [SMILES: CC(C)(C)[Si](C)(C)OCCC=C1CCOCC1]

To a solution of (3-((tert-butyldimethylsilyl)oxy)propyl)triphenylphosphonium, Bromide 2 (12.2 g, 23.976 mmol, 1.2 equiv) in THF (60 ml) was added potassium tert-butoxide (1.0M solution in THF, 26.56 ml, 26.56 mmol, 1.3 equiv) at 0° C. in drops and was stirred at 0° C. for 30 min. Then a solution of tetrahydro-4H-pyran-4-one 3 (2 g, 19.98 mmol, 1 equiv) in THF (10 ml) was added at 0° C. in drops and the reaction mixture was stirred at room temperature for 16 h. Upon completion of reaction (as confirmed by TLC analysis; 5% EtOAc in pet ether, Rf˜0.4), the reaction mixture was quenched with cold sat. NH₄Cl solution (15 mL) and extracted with MTBE (2×20 mL). The combined organic layer was washed with brine (50 mL), dried over Na₂SO₄, filtered and concentrated under vacuum to afford the crude which was purified by Isolera (Biotage R snap cartridge, KP-Sil, 50 g, 230-400 silica gel) using 4-6% of EtOAc in pet ether. The pure fractions of product were concentrated under vacuum to get tert-butyldimethyl(3-(tetrahydro-4H-pyran-4-ylidene)propoxy)silane 4 (1.5 g, 4.91 mmol, 49.2% yield) as colorless liquid.

¹H-NMR (400 MHz, DMSO-d6): δ 5.18 (t, J=7.20 Hz, 1H), 3.57-3.52 (m, 6H), 2.20-2.11 (m, 6H), 0.88 (s, 9H), 0.03 (t, J=3.20 Hz, 6H).

### Step-3: 3-(tetrahydro-4H-pyran-4-ylidene)propan-1-ol (5)

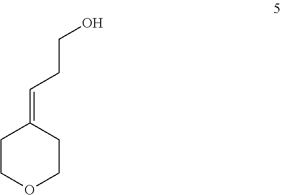

To a solution of tert-butyldimethyl(3-(tetrahydro-4H-pyran-4-ylidene)propoxy)silane 4 (1.5 g, 5.85 mmol, 1 equiv) in THF (30 ml) was added TBAF (1.0M sol in THF) (11.70 ml, 11.70 mmol, 2 equiv) at 0° C. and the resulting solution was stirred at room temperature for 16 h. Upon completion of reaction (as confirmed by TLC analysis; 50% EtOAc in pet ether, Rf˜0.2), the reaction mixture was concentrated under reduced pressure to afford the crude which was purified by Isolera (column size: Biotage R snap cartridge, KP-Sil, 50 g, 230-400 silica gel) using 60-80% of EtOAc in pet ether. The pure fractions of product were concentrated under vacuum to get 3-(tetrahydro-4H-pyran-4-ylidene)propan-1-ol 5 (820 mg, 5.75 mmol, 98% yield) as a colorless liquid.

¹H-NMR (400 MHz, DMSO-d6): δ 5.19 (t, J=7.60 Hz, 1H), 4.48 (t, J=5.60 Hz, 1H), 3.57-3.52 (m, 4H), 3.39-3.34 (m, 2H), 2.20-2.17 (m, 2H), 2.17-2.11 (m, 4H).

LCMS: 143.1 (M+H), Rt (min): 1.415, Area (%): 99.796. GCMS: 142.1, Rt (min): 6.643, Area (%): 95.66.

### Step-4: 3-(tetrahydro-4H-pyran-4-ylidene)propanal (6)

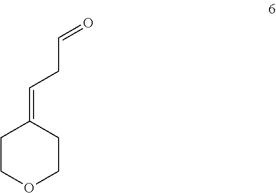

To a solution of 3-(tetrahydro-4H-pyran-4-ylidene)propan-1-ol 5 (300 mg, 2.110 mmol, 1 equiv) in DCM (10 ml) was added dess-martinperiodinane (1074 mg, 2.53 mmol, 1.2 equiv) at 0° C. in portions over 10 min and slowly warmed to room temperature and the reaction mixture was stirred at room temperature for 1 h. Upon completion of reaction (as confirmed by TLC analysis; 50% EtOAc in pet ether, Rf˜0.2), the reaction was quenched with a solution of aq. NaHCO₃/NaHSO₃ (1:1; 10 mL) and extracted with DCM (2×10 ml). The combined organic layer was washed with brine (15 mL), dried over anhydrous Na₂SO₄, filtered and concentrated under vacuum to afford the crude of 3-(tetrahydro-4H-pyran-4-ylidene)propanal 6 (400 mg, 2.85 mmol, 135% yield) as pale yellow gummy liquid which was taken as such for the next step.

### Step-5: tert-butyl ((1r,4r)-4-((2-(2,6-dioxopiperidin-3-yl)-1-oxoisoindolin-4-yl)(3-(tetrahydro-4H-pyran-4-ylidene)propyl)amino)cyclohexyl)carbamate (7)

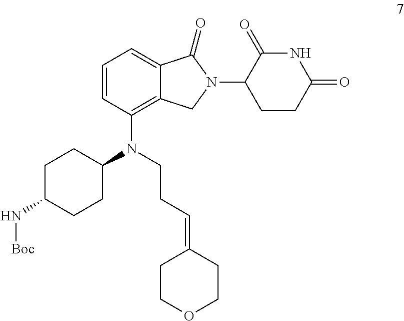 [SMILES: CC(C)(C)OC(=O)N[C@H]1CC[C@H](N(CCC=C2CCOCC2)c2cccc3c2CN(C2CCC(=O)NC2=O)C3=O)CC1]

To a stirred solution of tert-butyl ((1r,4r)-4-((2-(2,6-dioxopiperidin-3-yl)-1-oxoisoindolin-4-yl)amino)cyclohexyl)carbamate 88-3b (140 mg, 0.307 mmol, 1 equiv) in DCE (10 ml) and DMF (4 ml) was added 3-(tetrahydro-4H-pyran-4-ylidene)propanal 6 (344 mg, 2.453 mmol, 8 equiv), TFA (69.9 mg, 0.613 mmol, 2 equiv) and STAB (260 mg, 1.227 mmol, 4 equiv) at 0° C. and allowed to stir at room temperature for 16 h. Upon completion of reaction (as confirmed by LCMS) the reaction mixture was quenched with ice water (20 ml) and extracted with DCM (2×10 ml). The combined organic layer was concentrated under vacuum to give the crude product which was purified by preparative HPLC (Column: XSELECT C18-250, Method: 10 mm (NH₄)₂CO₃ in H₂O/ACN, Flow rate: 14 ml/min). The pure fractions of product were lyophilised to get tert-butyl ((1r,4r)-4-((2-(2,6-dioxopiperidin-3-yl)-1-oxoisoindolin-4-yl)(3-(tetrahydro-4H-pyran-4-ylidene)propyl)amino)cyclohexyl)carbamate 7 (36 mg, 0.059 mmol, 21.40% yield) as off white solid.

¹H-NMR (400 MHz, MeOD): δ 7.47 (q, J=5.20 Hz, 2H), 7.37 (q, J=2.40 Hz, 1H), 5.51 (s, 2H), 5.16 (q, J=5.20 Hz, 2H), 4.42 (s, 2H), 3.60-3.57 (m, 2H), 3.46-3.42 (m, 3H), 3.24 (q, J=6.80 Hz, 1H), 3.07 (s, 2H), 2.83 (t, J=2.40 Hz, 2H), 2.50 (q, J=Hz, 1H), 2.17 (t, J=4.00 Hz, 3H), 2.07 (q, J=12.40 Hz, 2H), 1.96 (q, J=4.80 Hz, 4H), 1.88 (d, J=3.20 Hz, 2H), 1.62 (d, J=12.80 Hz, 2H), 1.44 (s, 8H), 1.31-1.24 (m, 2H).

LCMS: 581.3 (M+H), Rt (min): 1.570, Area (%): 95.385. HPLC: RT (min): 6.019, Area (%): 95.131.

### Step-6: 3-(4-(((1r,4r)-4-aminocyclohexyl)(3-(tetrahydro-4H-pyran-4-ylidene)propyl)amino)-1-oxoisoindolin-2-yl)piperidine-2,6-dione. Formic Acid (344)

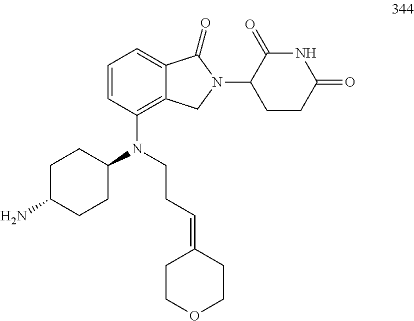 [SMILES: N[C@H]1CC[C@H](N(CCC=C2CCOCC2)c2cccc3c2CN(C2CCC(=O)NC2=O)C3=O)CC1]

To an ice cold solution of tert-butyl ((1r,4r)-4-((2-(2,6-dioxopiperidin-3-yl)-1-oxoisoindolin-4-yl)(3-(tetrahydro-4H-pyran-4-ylidene)propyl)amino)cyclohexyl)carbamate 7 (0.034 g, 0.059 mmol, 1 equiv) in DCM (2 ml) was added HCl (4 M in ethyl acetate, 0.293 ml, 1.171 mmol, 20 equiv) and was stirred for 1 h at room temperature. Upon completion of reaction (as confirmed by TLC (100% EtOAc, R_{f}˜0.0), the reaction mixture was concentrated under vacuum and the solid obtained was washed with MTBE (10 ml). The residue was dried under vacuum and lyophilized to get the crude product which was purified by preparative HPLC (Column: XSELECT C-18, Mobile phase: A: 0.1% FA in water, B: MeCN, Flow rate: 15 mL/min, RT:9.94 min) to get 3-(4-(((1r,4r)-4-aminocyclohexyl)(3-(tetrahydro-4H-pyran-4-ylidene)propyl)amino)-1-oxoisoindolin-2-yl)piperidine-2,6-dione, Formic Acid 344 (0.0105 g, 0.020 mmol, 64.6% yield)] as off-white solid.

¹H-NMR (400 MHz, DMSO-d₆): δ 8.381 (br s, 1H), 7.45-7.41 (m, 1H), 7.32-7.27 (m, 2H), 5.15-5.10 (m, 2H), 4.33-4.20 (m, 2H), 3.50-3.45 (m, 3H), 3.36-3.34 (m, 2H), 3.16-3.14 (m, 2H), 3.14-3.12 (m, 2H), 2.68-2.62 (m, 1H), 2.06-2.05 (m, 2H), 2.00-1.89 (m, 7H), 1.78-1.75 (m, 2H), 1.57-1.54 (m, 2H), 1.30-1.27 (m, 2H).

LCMS: 481.4 (M+H), Method: Mobile phase: A: 0.1% FA in H₂O, Mobile phase: B: ACN, Column: Atlantis dC18 (50*4.6) 5 μm, Flow Rate: 1.5 ml/min, Rt (min): 1.969, Area %:: 99.541.

HPLC: Method: Mobile phase: A: 0.1% FA in water, Mobile phase: B: ACN, Column: X-Bridge C8(50×4.6) mm, 3. 5 μm Flow: 2.0 mL/min, Rt (min): 2.197, Area %: 99.932.

### Compound 345

The compound having the following structure is prepared in analogy from the preparation procedure described for the above-mentioned compounds.

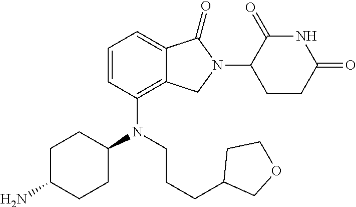 [SMILES: N[C@H]1CC[C@H](N(CCCC2CCOC2)c2cccc3c2CN(C2CCC(=O)NC2=O)C3=O)CC1]

### Hydrochloride Salt Form of Compound 346

### 3-(4-(((1r,4r)-4-aminocyclohexyl)(3-(tetrahydrofuran-3-yl)propyl)amino)-1-oxoisoindolin-2-yl)piperidine-2,6-dione, HCl

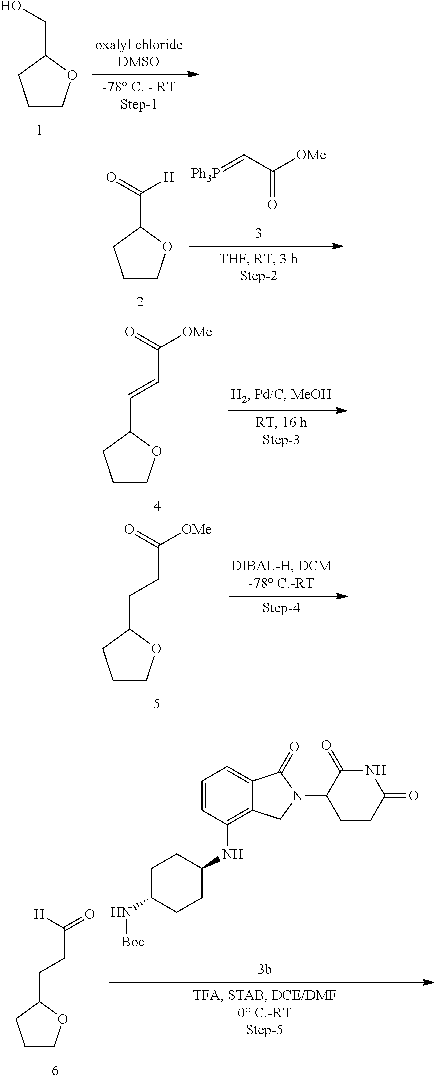

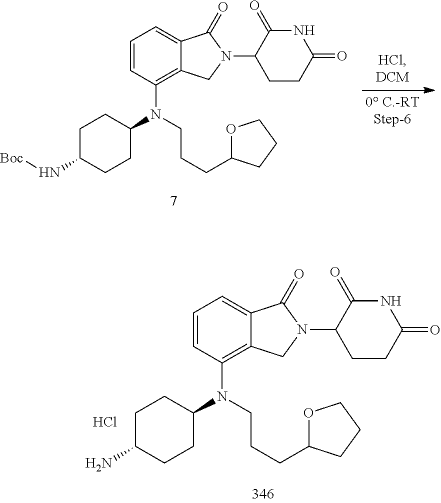 [SMILES: CC(C)(C)OC(=O)N[C@H]1CC[C@H](N(CCCC2CCCO2)c2cccc3c2CN(C2CCC(=O)NC2=O)C3=O)CC1 ; Cl ; N[C@H]1CC[C@H](N(CCCC2CCCO2)c2cccc3c2CN(C2CCC(=O)NC2=O)C3=O)CC1]

### Step-1: tetrahydrofuran-2-carbaldehyde

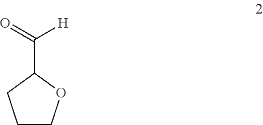

To a solution of oxalyl chloride (5.04 ml, 58.7 mmol, 3 equiv) in DCM (20 ml) was added DMSO (9.09 ml, 117 mmol, 6 equiv) in DCM (5 mL) at −78° C. and was allowed to stirred for 1 h. To this mixture, a solution of (tetrahydrofuran-2-yl) methanol 1 (2 g, 19.58 mmol, 1 equiv) in DCM (5 ml) was added and stirred for 1.5 h. Then trimethylamine (23.91 ml, 176 mmol, 9 equiv) was added and the reaction mixture was stirred for 15 minutes at −78° C. Upon completion of the reaction (as confirmed by TLC analysis, 50% EtOAc in pet ether, R_{f} of the product˜0.6). The reaction mixture was quenched with citric acid (30 mL) and extracted with DCM (2×80 ml). The organic layer was dried with sodium sulphate filtered and concentrated under reduced pressure to afford crude tetrahydrofuran-2-carbaldehyde 2 (2.0 g, 19.98 mmol, 102% yield) as dark brown liquid. The crude compound as such used for next step.

¹H-NMR (400 MHz, DMSO-d6): δ 9.56 (s, 1H), 5.04 (s, 1H), 3.82-3.79 (m, 2H), 3.08-3.05 (m, 1H), 1.78-1.93 (m, 3H).

### Step-2: methyl (E)-3-(tetrahydrofuran-2-yl) acrylate

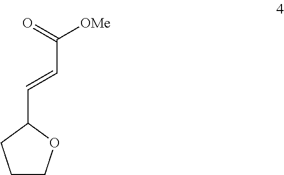 [SMILES: COC(=O)/C=C/C1CCCO1]

To a stirred solution of tetrahydrofuran-2-carbaldehyde 2 (2 g, 19.98 mmol, 1 equiv) in dry tetrahydrofuran (10 ml) was added methyl 2-(triphenyl-15-phosphanylidene) acetate 3 (7.35 g, 21.97 mmol, 1.1 equiv) at room temperature under nitrogen atmosphere. The mixture was stirred for overnight at room temperature. Upon completion of the reaction (as confirmed by TLC analysis, 20% EtOAc in pet ether, R_{f}˜0.8), the solvent was evaporated and hexane (50 ml) was added to the crude product, the mixture was stirred for 1 h and filtered. The filtrate was concentrated under reduced pressure to get crude product as colorless liquid. The crude compound was purified by column chromatography (Isolera, silica mesh size 230-400, flow rate 25 mL/min). The column was eluted using 30% ethyl acetate in pet ether to afford methyl (E)-3-(tetrahydrofuran-2-yl)acrylate 4 (1 g, 6.32 mmol, 31.6% yield) as pale yellow liquid.

LCMS: 157.1 [(M+H)], Method: Mobile phase: A: 0.1% TFA in H₂O, Mobile phase: B: 0.1% FA in ACN, Column: Atlantis dC18 (50×4.6 mm) 5 μm, Flow Rate: 1.5 ml/min Rt (min): 1.832, Area %: 98.637.

¹H-NMR (400 MHz, DMSO-d6): δ 6.87 (dd, J=4.80, 15.60 Hz, 1H), 5.97 (d, J=1.60 Hz, 1H), 4.48 (q, J=1.60 Hz, 1H), 3.86-3.82 (m, 1H), 3.82-3.79 (m, 3H), 2.13-2.05 (m, 2H), 1.88-1.81 (m, 2H), 1.56-1.65 (m, 2H).

### Step-3: methyl 3-(tetrahydrofuran-2-yl)propanoate

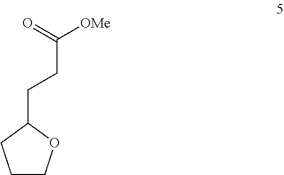 [SMILES: COC(=O)CCC1CCCO1]

To a solution of methyl (E)-3-(tetrahydrofuran-2-yl)acrylate 4 (0.47 g, 3.01 mmol, 1.0 equiv) in methanol (15.0 mL) was added Pd—C (10% wt/wt, 0.072 g, 0.060 mmol, 1 equiv) and then the mixture was stirred under hydrogen atmosphere for overnight. Upon completion of the reaction (TLC analysis, 20% EtOAc in pet ether, R_{f}˜0.3). The reaction mixture was filtered through Cellite bed and washed with MeOH (2×80 mL) and concentrated under reduced pressure to afford crude product methyl 3-(tetrahydrofuran-2-yl)propanoate 5 (0.13 g, 0.671 mmol, 16.12% yield) as colorless liquid.

¹H-NMR (400 MHz, CDCl₃): δ 3.74-3.68 (m, 2H), 3.59-3.541 (m, 4H), 2.37-2.322 (m, 2H), 1.92-1.87 (m, 1H), 1.80-1.76 (m, 2H), 1.70-1.64 (m, 2H), 1.41-1.38 (m, 1H).

### Step-4: 3-(tetrahydrofuran-2-yl)propanal

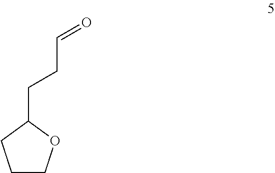

To a stirred solution of methyl 3-(tetrahydrofuran-2-yl)propanoate 5 (0.13 g, 0.822 mmol, 1.0 equiv) in dry dichloromethane (12.00 mL) at −78° C. under nitrogen atmosphere was added dropwise DIBAL-H (1.2 M sol in Toluene, 0.753 ml, 0.904 mmol, 1.1 equiv) under nitrogen atmosphere. The resultant solution was allowed to warm to room temperature and stirred for 20 min. Upon completion of the reaction (as confirmed by TLC analysis, 10% EtOAc in pet ether, R_{f} of the product˜0.3). The reaction mixture was quenched with saturated aqueous sodium potassium tartrate solution (10 mL) at 0° C., filtered through celite bed and wash the bed with DCM (1×25 mL). The organic layer was separated and the aqueous layer was extracted with DCM (2×30 mL). The combined organic layer was washed with brine (50 mL), dried over Na₂SO₄, filtered and concentrated under reduced pressure to afford the crude product 3-(tetrahydrofuran-2-yl)propanal 6 (0.15 g) as pale yellow liquid. The crude compound was used for next step without further purification.

### Step-5: tert-butyl ((1r,4r)-4-((2-(2,6-dioxopiperidin-3-yl)-1-oxoisoindolin-4-yl)(3-(tetrahydro-2H-pyran-4-yl)propyl)amino)cyclohexyl)carbamate

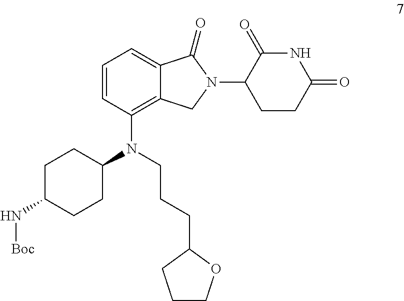 [SMILES: CC(C)(C)OC(=O)N[C@H]1CC[C@H](N(CCCC2CCCO2)c2cccc3c2CN(C2CCC(=O)NC2=O)C3=O)CC1]

To a stirred solution of tert-butyl ((1r,4r)-4-((2-(2,6-dioxopiperidin-3-yl)-1-oxoisoindolin-4-yl)amino)cyclohexyl)carbamate 3b (0.1 g, 0.219 mmol, 1.0 equiv) and 3-(tetrahydrofuran-2-yl)propanal 6 (0.168 g, 1.314 mmol, 6 equiv) in a mixture of DCE (4 mL) and DMF (2 mL) were added trifluoroacetic acid (0.075 g, 0.657 mmol, 3.0 equiv) and sodium triacetoxyborohydride (0.232 g, 1.095 mmol, 5.0 equiv) at 0° C. The resulting reaction mixture was stirred at room temperature for overnight. Upon completion of the reaction (as confirmed by LCMS analysis) the reaction mixture was poured into ice cold water (15 mL) and extracted with DCM (3×20 mL). The combined organic layer was washed with brine (50 mL), dried over anhydrous sodium sulphate, filtered and concentrated to get the crude product. The crude compound was purified by preparative HPLC (Column: Atlantis 250, 0.1% FA in ACN, flow rate: 15 mL/min) to afford tert-butyl ((1r,4r)-4-((2-(2,6-dioxopiperidin-3-yl)-1-oxoisoindolin-4-yl)(3-(tetrahydrofuran-2-yl)propyl)amino)cyclohexyl)carbamate 7 (50 mg, 0.087 mmol, 33.0% yield) as white solid.

¹H-NMR (400 MHz, DMSO-d₆): δ 10.97 (s, 1H), 7.41 (t, J=10.40 Hz, 1H), 7.31-7.24 (m, 2H), 6.68 (d, J=10.00 Hz, 1H), 5.11 (dd, J=4.80, 13.00 Hz, 1H), 4.35-4.20 (m, 2H), 3.62-3.66 (m, 2H), 3.51-3.57 (m, 1H), 3.32-3.48 (m, 3H), 3.14-3.11 (m, 2H), 2.62-2.51 (m, 2H), 2.02-2.00 (m, 1H), 1.81-1.70 (m, 7H), 1.53-1.44 (m, 2H), 1.40-1.36 (m, 11H), 1.31-1.20 (m, 5H),

LCMS: 569.5 [(M+H)], Method: Mobile phase: A: 0.1% FA in H₂O, Mobile phase: B: 0.1% TFA in ACN, Column: XBridge C18 (50×4.6 mm) 5 μm, Flow Rate: 1.5 ml/min Rt (min): 2.722, Area %: 99.64.

HPLC: Method: Mobile phase: A:0.1% FA in water, Mobile phase: B: MeCN, Column: Xbridge C8(50×4.6) mm, 3.5 μm, Flow:2.0 mL/min, Rt (min): 4.177 Area %: 99.874.

### Step-6:3-(4-(((1r,4r)-4-aminocyclohexyl)(3-(tetrahydrofuran-2-yl)propyl)amino)-1-oxoisoindolin-2-yl)piperidine-2,6-dione, HCl

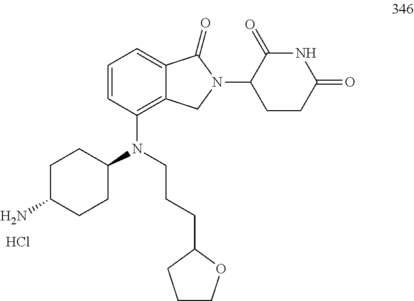 [SMILES: Cl ; N[C@H]1CC[C@H](N(CCCC2CCCO2)c2cccc3c2CN(C2CCC(=O)NC2=O)C3=O)CC1]

To a stirred solution of tert-butyl ((1r,4r)-4-((2-(2,6-dioxopiperidin-3-yl)-1-oxoisoindolin-4-yl)(3-(tetrahydrofuran-2-yl)propyl)amino)cyclohexyl)carbamate 7 (50 mg, 0.088 mmol, 1 equiv) in DCM (5 mL) was added hydrochloric acid (4 M sol in ethyl acetate) (2.231 ml, 8.92 mmol, 20 equiv) at 0° C. The resultant reaction mixture was allowed to room temperature and stirred for 2 h. Upon completion of the reaction (as confirmed by TLC analysis, 100% EtOAc, Rf of the product˜0.1), the reaction mixture was concentrated under reduced pressure to get the crude product as pale yellow gummy solid. The obtained solid was washed with MTBE (2×10 mL), dried under reduced pressure and lyophilized to afford 3-(4-(((1r,4r)-4-aminocyclohexyl)(3-(tetrahydrofuran-2-yl)propyl)amino)-1-oxoisoindolin-2-yl)piperidine-2,6-dione, HCl 346 (42 mg, 0.079 mmol, 90% yield) as off-white solid.

¹H-NMR (400 MHz, DMSO-d₆): δ 11.00 (s, 1H), 8.03 (s, 3H), 7.44-7.31 (m, 2H), 5.14-5.11 (m, 1H), 4.13-4.33 (m, 2H), 3.66-3.60 (m, 2H), 3.60-3.51 (m, 1H), 3.49-3.47 (m, 3H), 2.97-2.89 (m, 2H), 2.63-2.51 (m, 1H), 1.98-1.95 (m, 2H), 1.71-1.85 (m, 7H), 1.59-1.38 (m, 2H), 1.21-1.26 (m, 7H).

LCMS: 469.3 (M+H), Method: Mobile phase: A: 0.1% FA in H₂O, Mobile phase: B: ACN, Column: Atlantis dC18 (50×4.6 mm) 5 μm, Flow Rate:1.5 ml/min Rt (min): 1.539, Area %: 99.48.

HPLC: Method: Mobile phase: A: 0.1% TFA in water, Mobile phase: B: MeCN, Column: X Bridge C8 (50×4.6) mm, 3.5 μm, Flow: 2.0 mL/min, Rt (min): 2.031, Area %: 95.021.

### Compound 347

The compound having the following structure is prepared in analogy from the preparation procedure described for the above-mentioned compounds.

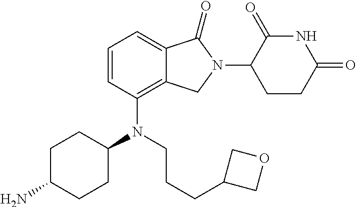 [SMILES: N[C@H]1CC[C@H](N(CCCC2COC2)c2cccc3c2CN(C2CCC(=O)NC2=O)C3=O)CC1]

### Compound 349

The compound having the following structure is prepared in analogy from the preparation procedure described for the above-mentioned compounds.

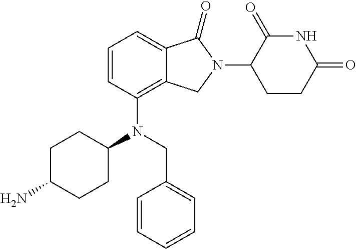 [SMILES: N[C@H]1CC[C@H](N(Cc2ccccc2)c2cccc3c2CN(C2CCC(=O)NC2=O)C3=O)CC1]

### Compound 352

The compound having the following structure is prepared in analogy from the preparation procedure described for the above-mentioned compounds.

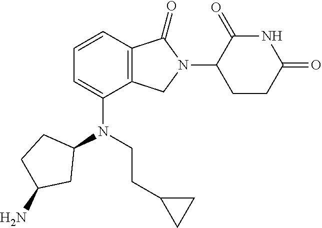 [SMILES: N[C@H]1CC[C@@H](N(CCC2CC2)c2cccc3c2CN(C2CCC(=O)NC2=O)C3=O)C1]

### Compound 353

The compound having the following structure is prepared in analogy from the preparation procedure described for the above-mentioned compounds.

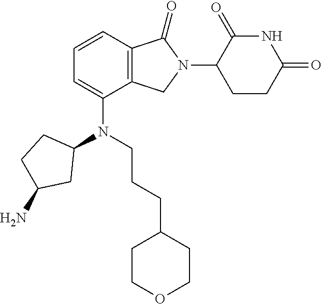 [SMILES: N[C@H]1CC[C@@H](N(CCCC2CCOCC2)c2cccc3c2CN(C2CCC(=O)NC2=O)C3=O)C1]

### Compound 354

The compound having the following structure is prepared in analogy from the preparation procedure described for the above-mentioned compounds.

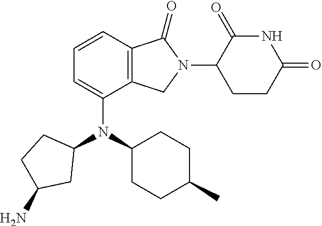 [SMILES: C[C@H]1CC[C@@H](N(c2cccc3c2CN(C2CCC(=O)NC2=O)C3=O)[C@@H]2CC[C@H](N)C2)CC1]

### Compound 355

The compound having the following structure is prepared in analogy from the preparation procedure described for the above-mentioned compounds.

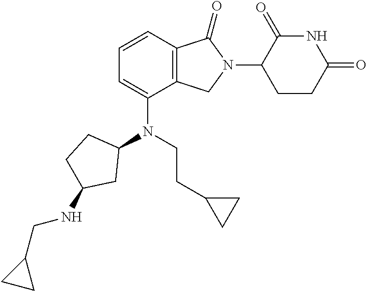 [SMILES: O=C1CCC(N2Cc3c(cccc3N(CCC3CC3)[C@@H]3CC[C@H](NCC4CC4)C3)C2=O)C(=O)N1]

### Compound 356

The compound having the following structure is prepared in analogy from the preparation procedure described for the above-mentioned compounds.

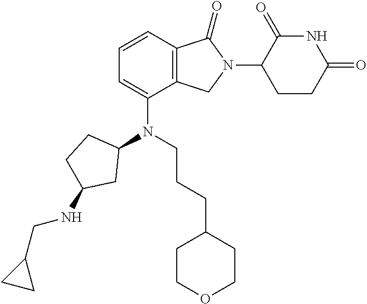 [SMILES: O=C1CCC(N2Cc3c(cccc3N(CCCC3CCOCC3)[C@@H]3CC[C@H](NCC4CC4)C3)C2=O)C(=O)N1]

### Compound 358

The compound having the following structure is prepared in analogy from the preparation procedure described for the above-mentioned compounds.

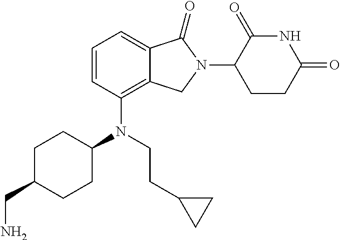 [SMILES: NC[C@H]1CC[C@@H](N(CCC2CC2)c2cccc3c2CN(C2CCC(=O)NC2=O)C3=O)CC1]

### Compound 359

### (1S,3R)-3-((2-cyclopropylethyl)(2-(2,6-dioxopiperidin-3-yl)-1-oxoisoindolin-4-yl)amino)cyclopentane-1-carboxamide

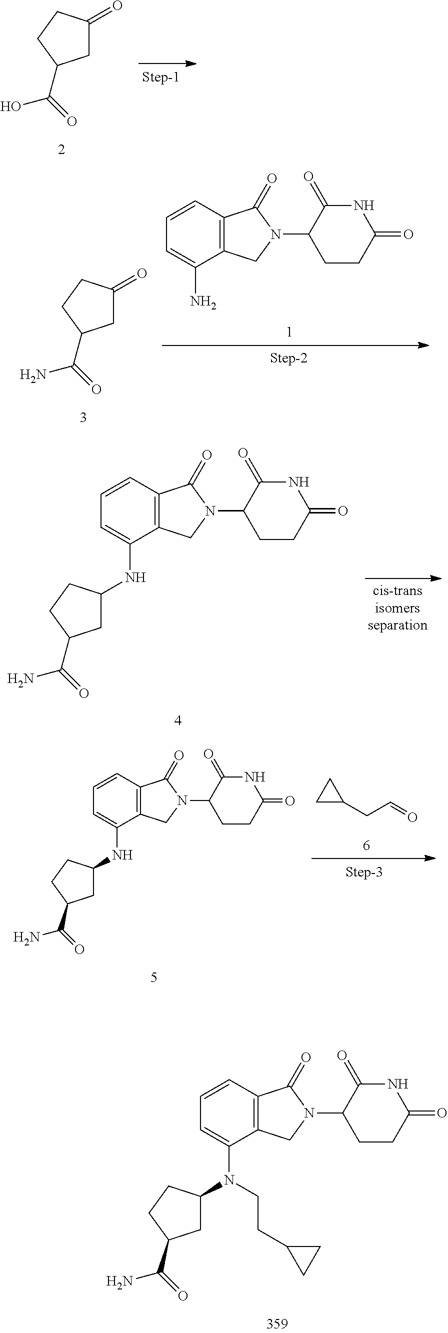

### Step-1: 3-oxocyclopentane-1-carboxamide (3)

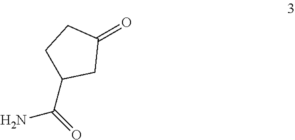

To an ice cold solution of 3-oxocyclopentane-1-carboxylic acid 2 (1 g, 7.80 mmol, 1 equiv) in DCM (2 mL) was added oxalyl chloride (1.981 g, 15.61 mmol, 2 equiv) dropwise followed by catalytic amount of DMF (1 drops) and stirred for 2 h at room temperature. Upon completion of the reaction (as confirmed by TLC analysis, 100% EtOAc in pet ether, R_{f}˜0.8, as methyl ester), the reaction mixture was distilled under reduced pressure. The resulted acid chloride mixture was dissolved in THF (1.5 mL) and added dropwise to a pre-cooled solution of ammonia (0.5 M in THF) (18.73 ml, 9.37 mmol, 2.4 equiv) at −78° C. The reaction mixture was warmed to room temperature and stirred for 16 h. Upon completion of the reaction (as confirmed by TLC analysis, 100% EtOAc in pet ether, R_{f}˜0.1), the reaction mixture was concentrated and resulted residue was poured into ice cold solution of sat. NaHCO₃ solution (50 mL) and extracted with EtOAc (2×50 mL). The combined organic layer was washed with brine (50 mL), dried over Na₂SO₄, filtered and concentrated to give the crude product. As product was not completely extracted from the aqueous layer, it was combined with the crude and purified by reverse-phase column chromatography (Grace column: C18 40 μm, 40 g; flow rate: 60 mL/min; 0.1% aqueous HCOOH/ACN mobile phase). The pure fractions were concentrated and neutralized with 10% NaHCO₃ solution (10 mL) and extracted with EtOAc (3×50 mL). The combined organic layer was dried over Na₂SO₄, filtered and concentrated under reduced pressure to get 3-oxocyclopentane-1-carboxamide 3 (630 mg, 4.38 mmol, 56.2% yield) as brown solid.

¹H-NMR (400 MHz, DMSO-d6): δ 7.44 (s, 1H), 6.89 (d, J=11.60 Hz, 1H), 2.94 (q, J=4.80 Hz, 1H), 2.24 (q, J=8.40 Hz, 2H), 2.18-2.08 (m, 3H), 1.92-1.83 (m, 1H), 1.36 (s, 1H).

LCMS: 128.1 (M+H), Rt (min)=0.734, Area (%): 98.258.

### Step-2: (1R,3R)-3-((2-(2,6-dioxopiperidin-3-yl)-1-oxoisoindolin-4-yl)amino)cyclopentane-1-carboxamide (5)

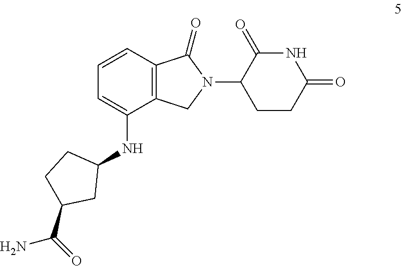 [SMILES: NC(=O)[C@H]1CC[C@@H](Nc2cccc3c2CN(C2CCC(=O)NC2=O)C3=O)C1]

To a mixture of 3-(4-amino-1-oxoisoindolin-2-yl)piperidine-2,6-dione 1 (1.05 g, 4.05 mmol, 1 equiv) and 3-oxocyclopentane-1-carboxamide 3 (0.618 g, 4.86 mmol, 1.2 equiv) in a mixture of DCE (10 ml) and DMF (10 ml) was added TFA (1.386 g, 12.14 mmol, 3 equiv) and STAB (2.576 g, 12.14 mmol, 3 equiv) at 0° C. The resulting reaction mixture was stirred at room temperature for 24 h. Upon completion of reaction (as confirmed by LCMS analysis), the reaction mixture was concentrated under reduced pressure to afford the crude product with mixture of isomers 4 which was purified by preparative HPLC (Column: XBRIDGE C18-150, 500 ul, Mobile phase: A: 0.1% FA in water, B: MeCN, Flow rate: 15 mL/min, Retention time: 10.1 min) to afford (1R,3R)-3-((2-(2,6-dioxopiperidin-3-yl)-1-oxoisoindolin-4-yl)amino)cyclopentane-1-carboxamide 5 (250 mg, 0.660 mmol, 48.3% yield) as dark brown solid.

¹H-NMR (400 MHz, DMSO-d6): δ 11.00 (s, 1H), 8.14 (s, 1H), 7.37 (s, 1H), 7.28 (t, J=10.40 Hz, 1H), 6.93 (d, J=9.60 Hz, 1H), 6.80 (q, J=10.80 Hz, 2H), 5.79 (t, J=10.00 Hz, 1H), 5.14-5.07 (m, 1H), 4.26-4.08 (m, 2H), 3.90 (d, J=8.40 Hz, 1H), 2.92 (s, 1H), 2.73-2.58 (m, 2H), 2.44-2.21 (m, 1H), 2.19-2.01 (m, 1H), 1.87-1.61 (m, 5H).

LCMS: 371.1 (M+H), Rt (min)=1.595, Area (%): 97.773.

### Step-3: (1S,3R)-3-((2-cyclopropylethyl)(2-(2,6-dioxopiperidin-3-yl)-1-oxoisoindolin-4-yl)amino)cyclopentane-1-carboxamide (359)

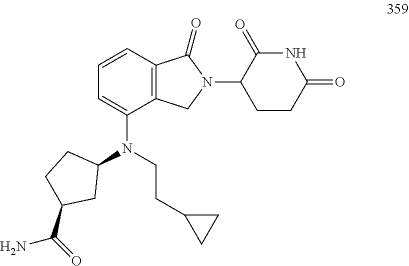 [SMILES: NC(=O)[C@H]1CC[C@@H](N(CCC2CC2)c2cccc3c2CN(C2CCC(=O)NC2=O)C3=O)C1]

To a mixture of (1S,3R)-3-((2-(2,6-dioxopiperidin-3-yl)-1-oxoisoindolin-4-yl)amino)cyclopentane-1-carboxamide 5 (0.110 g, 0.297 mmol, 1 equiv) and 2-cyclopropylacetaldehyde 6 (0.250 g, 2.97 mmol, 10 equiv) in a mixture of DCE (2 ml) and DMF (1 ml) was added TFA (0.069 ml, 0.891 mmol, 3 equiv) at 0° C. The reaction mixture was stirred at 0° C. for 30 min, STAB (0.315 g, 1.485 mmol, 5 equiv) was added to the reaction mixture at 0° C. and stirred at room temperature for 3 h. Upon completion of reaction (as confirmed by LCMS) the reaction mixture was quenched with ice cold water (15 mL) and extracted with EtOAc (2×20 mL). The combined organic layer was washed with brine (1×50 mL), dried over Na₂SO₄, filtered and concentrated under reduced pressure to afford the crude product which was purified by preparative HPLC (Column: XBridge-C18 19.1×250 mm, 500 ul, Mobile phase: A: 10 mm ammonium acetate in water/, B: MeCN, Flow rate: 15 mL/min, Retention time: 10.1 min). to afford (1S,3R)-3-((2-cyclopropylethyl)(2-(2,6-dioxopiperidin-3-yl)-1-oxoisoindolin-4-yl)amino)cyclopentane-1-carboxamide 359 (0.045 g, 0.103 mmol, 12.85% yield) as off-white solid.

¹H-NMR (400 MHz, DMSO-d₆): δ 10.97 (br s, 1H), 7.42 (t, J=7.60 Hz, 1H), 7.33-7.30 (m, 2H), 7.22 (br s, 1H), 6.72 (br s, 1H), 5.13-5.08 (m, 1H), 4.38 (d, J=17.20 Hz, 1H), 4.26 (d, J=17.20 Hz, 1H), 3.81-3.71 (m, 1H), 3.22-3.18 (m, 2H), 2.91 (m, 1H), 2.61-2.54 (m, 3H), 2.00-1.91 (m, 2H), 1.76-1.71 (m, 4H), 1.68-1.65 (m, 1H), 1.18-1.15 (m, 2H), 0.60 (m, 1H), 0.35-0.31 (m, 2H), −0.05-0.09 (m, 2H).

LCMS: 439.1 (M+H). Method: Column: XBridge C8 (50×4.6 mm) 3.5 μm, Mobile phase A: 0.1% TFA in H₂O, B: ACN, Flow Rate: 2.0 mL/min, Rt (min):0.994, Area (%): 99.868.

HPLC: Method: A: 0.1% TFA in H₂O, B: ACN, Column: XBridge C8 (50×4.6 mm) 3.5 μm; Flow Rate: 2.0 mL/min; Rt (min): 2.142; Area (%): 99.945.

### Compound 360

### (1S,3R)-3-((2-cyclopropylethyl)(2-(2,6-dioxopiperidin-3-yl)-1-oxoisoindolin-4-yl)amino)-N-methylcyclopentane-1-carboxamide

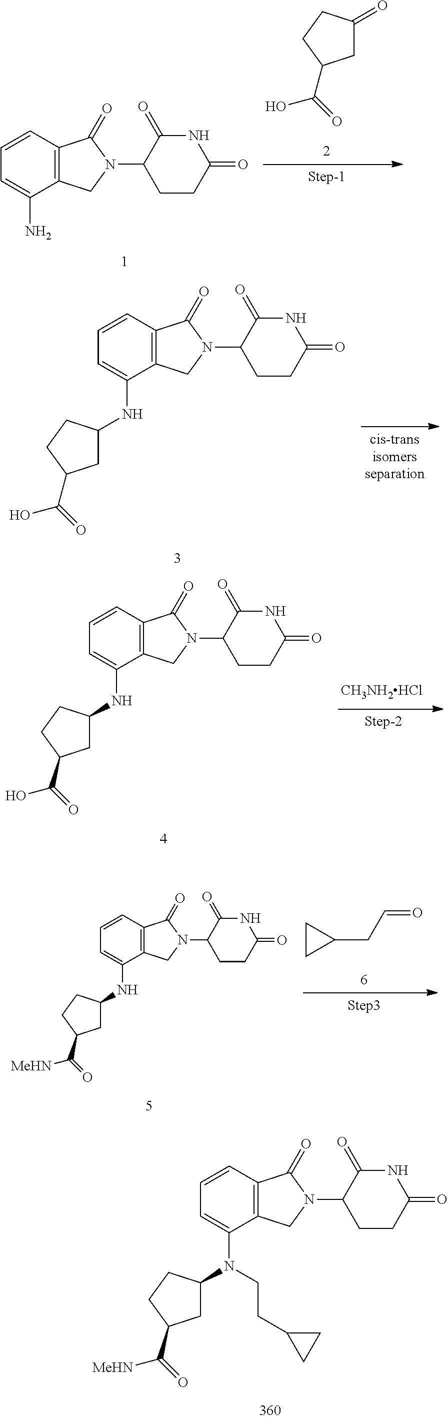

### Step-1: (1S,3R)-3-((2-(2,6-dioxopiperidin-3-yl)-1-oxoisoindolin-4-yl)amino)cyclopentane-1-carboxylic acid (4)

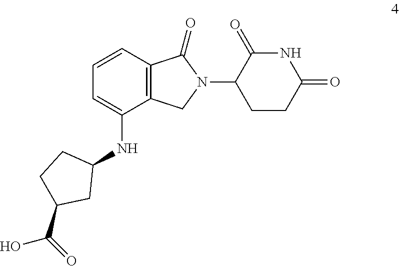 [SMILES: O=C1CCC(N2Cc3c(N[C@@H]4CC[C@H](C(=O)O)C4)cccc3C2=O)C(=O)N1]

To a stirred solution of 3-(4-amino-1-oxoisoindolin-2-yl)piperidine-2,6-dione 1 (1.5 g, 5.79 mmol, 1 equiv) and 3-oxocyclopentane-1-carboxylic acid 2 (1.483 g, 11.57 mmol, 2 equiv) in a mixture of DCE (30 ml) and DMF (10 ml) was added TFA (1.979 g, 17.36 mmol, 3 equiv) followed by STAB (6.13 g, 28.9 mmol, 5 equiv) at 0° C. The resulting solution was stirred at room temperature for 16 h. Upon completion of reaction (as confirmed by TLC, 95% EtOAc in 5% pet-ether, Rf˜0.05), the reaction mixture was concentrated under reduced pressure to give the crude product with mixture of isomers which was purified by preparative HPLC (Column: YMC TRIART 150 mm, 600 ul, Mobile phase: A: 0.1% TFA in water, B: MeCN, Flow rate: 15 mL/min, Retention time: 14.0 min) to get two fractions. The first fraction was lyophilized to afford undesired isomer of (1R,3R)-3-((2-(2,6-dioxopiperidin-3-yl)-1-oxoisoindolin-4-yl)amino)cyclopentane-1-carboxylic acid (0.520 g) as off-white solid. The second fraction was lyophilized to afford the desired isomer of (1S,3R)-3-((2-(2,6-dioxopiperidin-3-yl)-1-oxoisoindolin-4-yl)amino)cyclopentane-1-carboxylic acid 4 (0.640 g) as off-white solid. Analysis for (1R,3R)-3-((2-(2,6-dioxopiperidin-3-yl)-1-oxoisoindolin-4-yl)amino)cyclopentane-1-carboxylic acid (undesired isomer):

¹H-NMR (400 MHz, DMSO-d6): δ 11.00 (s, 1H), 7.29 (s, 1H), 6.95 (s, 1H), 6.76 (s, 1H), 5.12 (t, J=6.40 Hz, 1H), 4.28-4.16 (m, 2H), 3.93 (t, J=6.80 Hz, 1H), 2.93-2.88 (m, 2H), 2.65 (s, 1H), 2.50-2.44 (m, 1H), 2.27-1.98 (m, 4H), 1.84-1.71 (m, 2H), 1.58 (q, J=10.40 Hz, 1H).

LCMS: 372.1 (M+H), Rt (min)=1.079, Area (%): 98.829. HPLC: Rt (min): 2.312 & 2.402, Area (%): 57.826 & 41.677.

Analysis for (1S,3R)-3-((2-(2,6-dioxopiperidin-3-yl)-1-oxoisoindolin-4-yl)amino)cyclopentane-1-carboxylic acid (4) desired isomer

¹H-NMR (400 MHz, DMSO-d6): δ 12.45 (s, 1H), 11.02 (s, 1H), 7.29 (t, J=7.60 Hz, 1H), 6.94 (d, J=7.20 Hz, 1H), 6.79 (d, J=8.00 Hz, 1H), 5.12 (q, J=5.20 Hz, 1H), 4.23 (t, J=5.60 Hz, 1H), 4.13 (q, J=3.20 Hz, 1H), 3.88 (t, J=6.80 Hz, 2H), 2.93 (s, 1H), 2.77 (t, J=8.40 Hz, 1H), 2.62 (d, J=16.80 Hz, 1H), 2.34-2.27 (m, 3H), 2.05-1.99 (m, 2H), 1.88 (q, J=8.00 Hz, 2H), 1.73 (s, 1H), 1.57 (s, 1H).

LCMS: 372.1 (M+H), Rt (min)=1.168, Area (%): 99.681. HPLC: Rt (min)=2.794, Area (%): 95.850.

### Step-2: (1S,3R)-3-((2-(2,6-dioxopiperidin-3-yl)-1-oxoisoindolin-4-yl)amino)-N-methylcyclopentane-1-carboxamide (5)

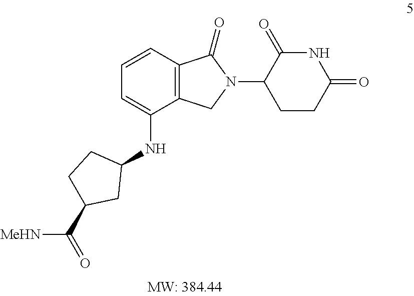 [SMILES: CNC(=O)[C@H]1CC[C@@H](Nc2cccc3c2CN(C2CCC(=O)NC2=O)C3=O)C1]

To a mixture of (1S,3R)-3-((2-(2,6-dioxopiperidin-3-yl)-1-oxoisoindolin-4-yl)amino)cyclopentane-1-carboxylic acid 4 (0.3 g, 0.808 mmol, 1 equiv) and methylamine (2.0M sol in THF) (4.04 ml, 8.08 mmol, 10 equiv) in DMF (10 ml) was added DIPEA (0.431 ml, 2.423 mmol, 3 equiv) followed by HATU (0.369 g, 0.969 mmol, 1.2 equiv) at 0° C. and the resulting solution was stirred for 16 h at room temperature. Upon completion of reaction (as confirmed by LCMS analysis), the reaction mixture was concentrated under reduced pressure to give the crude product which was purified by reverse-phase column chromatography (Grace column: C18 40 μm, 120 g; flow rate: 20 mL/min; 0.1% aqueous TFA/MeCN mobile phase). The pure fractions of product were concentrated under reduced pressure to afford (1S,3R)-3-((2-(2,6-dioxopiperidin-3-yl)-1-oxoisoindolin-4-yl)amino)-N-methylcyclopentane-1-carboxamide 5 (0.230 g, 0.568 mmol, 68.3% yield) as pale beige solid.

¹H-NMR (400 MHz, DMSO-d6): δ 11.01 (s, 1H), 7.82 (d, J=6.00 Hz, 1H), 7.28 (t, J=10.40 Hz, 1H), 6.93 (d, J=9.60 Hz, 1H), 6.79 (d, J=10.80 Hz, 1H), 5.14-5.07 (m, 2H), 4.19 (q, J=22.80 Hz, 2H), 3.92 (q, J=8.80 Hz, 1H), 2.89 (q, J=9.20 Hz, 2H), 2.66 (d, J=10.80 Hz, 1H), 2.58 (d, J=6.00 Hz, 4H), 2.34 (s, 1H), 2.28 (t, J=2.40 Hz, 1H), 1.91 (s, 1H), 1.79-1.73 (m, 2H), 1.71-1.64 (m, 2H). LCMS: 385.1 (M+H), Rt (min)=1.188, Area (%): 95.003.

### Step-3: (1S,3R)-3-((2-cyclopropylethyl)(2-(2,6-dioxopiperidin-3-yl)-1-oxoisoindolin-4-yl)amino)-N-methylcyclopentane-1-carboxamide (360)

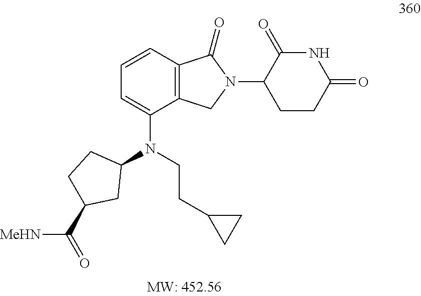 [SMILES: CNC(=O)[C@H]1CC[C@@H](N(CCC2CC2)c2cccc3c2CN(C2CCC(=O)NC2=O)C3=O)C1]

To a mixture of (1S,3R)-3-((2-(2,6-dioxopiperidin-3-yl)-1-oxoisoindolin-4-yl)amino)-N-methylcyclopentane-1-carboxamide 5 (0.15 g, 0.390 mmol, 1 equiv) and 2-cyclopropylacetaldehyde (0.328 g, 3.90 mmol, 10 equiv) in a mixture of DCM (2 ml) and DMF (1 ml) was added TFA (0.090 ml, 1.171 mmol, 3 equiv) at 0° C. and the resulting reaction mixture was stirred at 0° C. for 30 min. STAB (0.413 g, 1.951 mmol, 5 equiv) was added to the reaction mixture at 0° C. and stirred for 16 h at room temperature. Upon completion of reaction (as confirmed by LCMS analysis), the reaction mixture was quenched with ice cold water (25 mL) and extracted with EtOAc (2×20 mL). The combined organic layer was washed with brine (1×50 mL), dried over Na₂SO₄, filtered and concentrated under reduced pressure to afford the crude product which was purified by preparative HPLC (Column: X Bridge C18-150, 500 ul, Mobile phase: A: 10 mm NH₄OAc in water, B: MeCN, Flow rate: 15 mL/min, Retention time: 9.0 min) to afford (1S,3R)-3-((2-cyclopropylethyl)(2-(2,6-dioxopiperidin-3-yl)-1-oxoisoindolin-4-yl)amino)-N-methylcyclopentane-1-carboxamide 360 (0.032 g, 0.069 mmol, 37.1% yield)] as white solid.

¹H-NMR (400 MHz, DMSO-d₆): δ 10.98 (br s, 1H), 7.70-7.67 (m, 1H), 7.42 (t, J=7.60 Hz, 1H), 7.31 (t, J=8.00 Hz, 2H), 5.13-5.08 (m, 1H), 4.38 (d, J=17.20 Hz, 1H), 4.26 (d, J=17.60 Hz, 1H), 3.69-3.79 (m, 1H), 3.22-3.18 (m, 2H), 2.91 (m, 1H), 2.56-2.51 (m, 5H), 1.92-1.91 (m, 2H), 1.76-1.69 (m, 4H), 1.69-1.66 (m, 2H), 1.18-1.15 (m, 2H), 0.60 (m, 1H), 0.36-0.31 (m, 2H), −0.05-−0.08 (m, 2H).

LCMS: 453.2 (M+H). Method: Column: XBridge C8 (50×4.6 mm) 3.5 μm, Mobile phase A: 0.1% TFA in H₂O, B: ACN, Flow Rate: 2.0 mL/min, Rt (min): 1.106, Area (%): 97.491.

HPLC: Method: A: 0.1% TFA in H₂O, B: ACN, Column: XBridge C8 (50×4.6 mm) 3.5 μm; Flow Rate: 2.0 mL/min; Rt (min): 2.376; Area (%): 98.149.

### Compound 361

### (1S,3R)-3-((2-(2,6-dioxopiperidin-3-yl)-1-oxoisoindolin-4-yl)(3-(tetrahydro-2H-pyran-4-yl)propyl)amino)cyclopentane-1-carboxamide

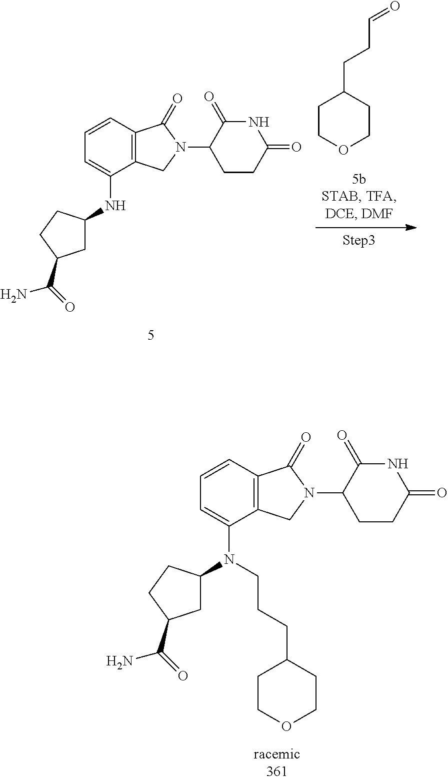

To a mixture of (1S,3R)-3-((2-(2,6-dioxopiperidin-3-yl)-1-oxoisoindolin-4-yl)amino)cyclopentane-1-carboxamide 5 (0.125 g, 0.337 mmol, 1 equiv) and 3-(tetrahydro-2H-pyran-4-yl)propanal 5b (0.480 g, 3.37 mmol, 10 equiv) in DCE (2 ml) and DMF (1 ml) was added TFA (0.078 ml, 1.012 mmol, 3 equiv) at 0° C. The reaction mixture was stirred at 0° C. for 30 min. Then STAB (0.358 g, 1.687 mmol, 5 equiv) was added at 0° C. and stirred at room temperature for 16 h. Upon completion of reaction (as confirmed by LCMS analysis) the reaction mixture was concentrated under reduced pressure to give the crude product which was purified by preparative HPLC (Column: XBRIDGE C18-150, 500 ul, Mobile phase: A: 10 mm NH₄OAc in water, B: MeCN, Flow rate: 15 mL/min, Retention time: 8.0 min). The pure fractions of product were lyophilized to afford the desired product with residual solvent peak as observed in 1HNMR, it was co-distilled with water (2×6 mL) and lyophilized to afford (1S,3R)-3-((2-(2,6-dioxopiperidin-3-yl)-1-oxoisoindolin-4-yl)(3-(tetrahydro-2H-pyran-4-yl)propyl)amino)cyclopentane-1-carboxamide 361 (0.060 g, 0.120 mmol, 53.0% yield) as off-white solid.

¹H-NMR (400 MHz, DMSO-d₆): δ 10.98 (br s, 1H), 7.46-7.42 (m, 1H), 7.35-7.32 (m, 2H), 7.22 (br s, 1H), 6.72 (br s, 1H), 5.13-5.09 (m, 1H), 4.36-4.35 (m, 1H), 4.29-4.28 (m, 1H), 3.77-3.74 (m, 3H), 3.22-3.19 (m, 2H), 3.09-3.08 (m, 2H), 3.06-2.91 (m, 1H), 2.61-2.52 (m, 3H), 2.01-2.00 (m, 2H), 1.74-1.70 (m, 3H), 1.67-1.62 (m, 2H), 1.45-1.41 (m, 2H), 1.33-1.25 (m, 3H), 1.16-1.13 (m, 2H), 1.04-1.01 (m, 2H).

LCMS: 497.0 (M+H). Method: Column: Atlantis dC18 (50×4.6 mm) 5 μm, Mobile phase A: 0.1% FA in H₂O, B: ACN, Flow Rate: 1.5 mL/min, Rt (min): 1.550, Area (%): 96.226.

HPLC: Method: A: 0.1% TFA in H₂O, B: ACN, Column: X Bridge C8 (50×4.6 mm) 3.5 μm; Flow Rate: 2.0 mL/min; Rt (min): 2.061; Area (%): 99.703.

### Compound 362

### (1S,3R)-3-((2-(2,6-dioxopiperidin-3-yl)-1-oxoisoindolin-4-yl)(3-(tetrahydro-2H-pyran-4-yl)propyl)amino)-N-methylcyclopentane-1-carboxamide

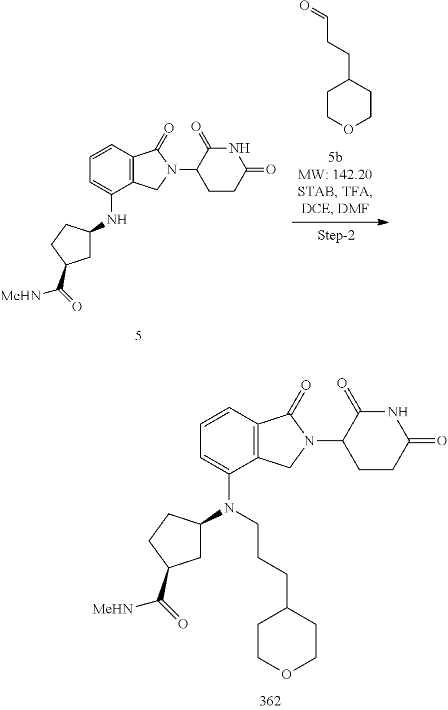 [SMILES: CNC(=O)[C@H]1CC[C@@H](Nc2cccc3c2CN(C2CCC(=O)NC2=O)C3=O)C1 ; O=CCCC1CCOCC1 ; CNC(=O)[C@H]1CC[C@@H](N(CCCC2CCOCC2)c2cccc3c2CN(C2CCC(=O)NC2=O)C3=O)C1]

To a stirred solution of (1S,3R)-3-((2-(2,6-dioxopiperidin-3-yl)-1-oxoisoindolin-4-yl)amino)-N-methylcyclopentane-1-carboxamide 5 (0.07 g, 0.182 mmol, 1 equiv) and 3-(tetrahydro-2H-pyran-4-yl)propanal 5b (0.259 g, 1.821 mmol, 10 equiv) in a mixture of DCE (2 ml) and DMF (1 ml) was added TFA (0.042 ml, 0.546 mmol, 3 equiv) at 0° C. and the resulting reaction mixture was stirred at 0° C. for 30 min. STAB (0.193 g, 0.910 mmol, 5 equiv) was added to the reaction mixture at 0° C. and stirred for 36 h at room temperature. Upon completion of reaction (as confirmed by LCMS analysis), the reaction mixture was quenched with ice-cold water (15 mL) and extracted with EtOAc (2×20 mL). The combined organic layer was washed with brine (1×50 mL), dried over Na₂SO₄, filtered and concentrated under reduced pressure to afford the crude product which was purified by preparative HPLC (Column: XBridge C18-150, 500 ul, Mobile phase: A: 10 mm NH₄OAc in water, B: MeCN, Flow rate: 15 mL/min, Retention time: 8.0 min) to afford (1S,3R)-3-((2-(2,6-dioxopiperidin-3-yl)-1-oxoisoindolin-4-yl)(3-(tetrahydro-2H-pyran-4-yl)propyl)amino)-N-methylcyclopentane-1-carboxamide 362 (0.056 g, 0.109 mmol, 36.6% yield) as off-white solid.

¹H-NMR (400 MHz, DMSO-d₆): δ 10.98 (br s, 1H), 7.68-7.67 (m, 1H), 7.44 (t, J=7.60 Hz, 1H), 7.35-7.32 (m, 2H), 5.12-5.09 (m, 1H), 4.36-4.28 (m, 2H), 3.78-3.74 (m, 3H), 3.22-3.16 (m, 2H), 3.09-3.08 (m, 2H), 2.96-2.85 (m, 1H), 2.68-2.51 (m, 5H), 1.98-1.92 (m, 1H), 1.90-1.83 (m, 1H), 1.73-1.65 (m, 5H), 1.45-1.41 (m, 2H), 1.40-1.21 (m, 4H), 1.29-1.13 (m, 2H), 1.04-0.99 (m, 2H).

LCMS: 511.2 (M+H). Method: Column: Zorbax Extend C18 (50×4.6 mm) 5 μm, Mobile phase A: 10 mM Ammonium acetate in H₂O, B: ACN, Flow Rate: 1.2 mL/min, Rt (min): 1.856, Area (%): 99.757.

HPLC: Method: A: 10 mM Ammonium acetate in H₂O, B: ACN, Column: XBridge C8 (50×4.6 mm) 3.5 m; Flow Rate: 1.0 mL/min; Rt (min): 3.975; Area (%): 99.012.

### Compound 512

### 3-(4-(((1r,4r)-4-((3,3-difluorocyclobutyl)amino)cyclohexyl)(4,4,4-trifluorobutyl)amino)-1-oxoisoindolin-2-yl)piperidine-2,6-dione

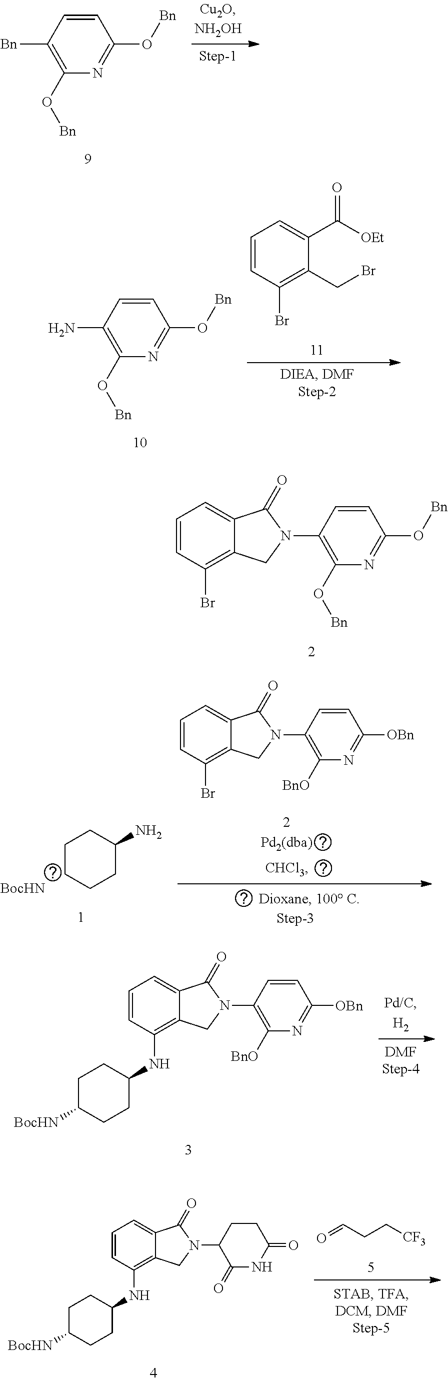

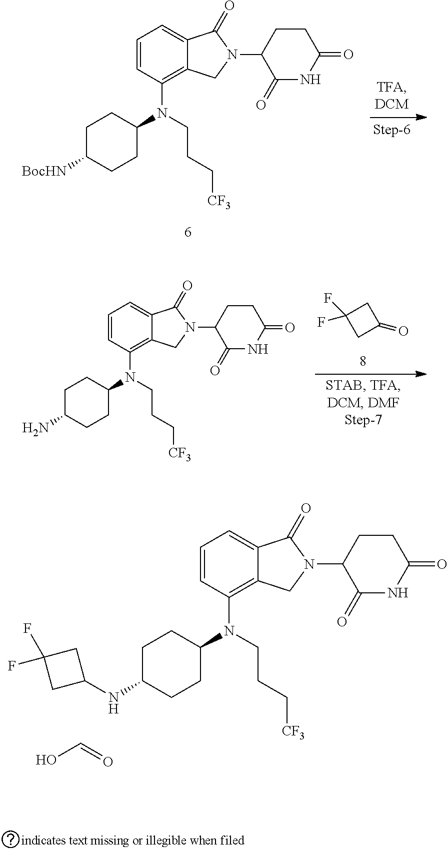

### Step-1: 2,6-bis(benzyloxy)pyridin-3-amine

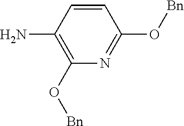 [SMILES: Nc1ccc(OCc2ccccc2)nc1OCc1ccccc1]

To a stirred mixture of 2,6-bis(benzyloxy)-3-bromopyridine 9 (10 g, 27.009 mmol, 1 equiv) and Cu₂O (100 g, 698.856 mmol, 25 equiv) in NMP (50 mL, 518.495 mmol, 19.20 equiv) were added NH₄OH (50.00 mL, 1284.027 mmol, 47.54 equiv) at room temperature. The resulting mixture was stirred for overnight at 80° C. The filtrate was collected by filtration. The filtrate was purified by silica gel column chromatography, eluted with PE/EA (5:1) to afford 2,6-bis(benzyloxy)pyridin-3-amine 10 (7.5 g, 90.64%) as yellow oil.

LCMS: 307.14 (M+H). Method: Mobile Phase A: Water/0.1% FA. Mobile Phase B: Acetonitrile. Flow rate: 1.5 mL/min. COLUMN: HPH (50×3.0) mm, 2.7μ. Rt (min): 1.121; Area %—98.629.

### Step-2: 2-[2,6-bis(benzyloxy)pyridin-3-yl]-4-bromo-3H-isoindol-1-one

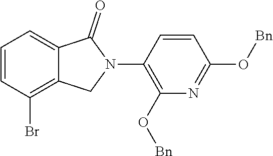 [SMILES: O=C1c2cccc(Br)c2CN1c1ccc(OCc2ccccc2)nc1OCc1ccccc1]

To a stirred solution of 2,6-bis(benzyloxy)pyridin-3-amine 10 (7.00 g, 22.855 mmol, 1.1 equiv) in anhydrous DMF (105.00 mL, 1356.762 mmol, 65.30 equiv) was added ethyl 3-bromo-2-(bromomethyl)benzoate 11 (6.69 g, 20.777 mmol, 1.00 equiv) and DIEA (8.06 g, 62.331 mmol, 3 equiv) at room temperature. The reaction mixture was stirred at 100° C. for a period of overnight. After completion of reaction, the reaction mixture was quenched by addition of water 150 mL. The aqueous layer was extracted with ethyl acetate (300 mL). The combined organic phase was washed with brine (150 mL), dried over anhydrous sodium sulfate and concentrated under reduced pressure to give crude product which was further purified by column chromatography using PE in ethyl acetate (5-20: 1) to afford desired compound 2-[2,6-bis(benzyloxy)pyridin-3-yl]-4-bromo-3H-isoindol-1-one 2 (6.00 g, 57.60%) as brown yellow solid.

LCMS: 501.07 (M+H). Method: Mobile Phase A: Water/0.1% FA. Mobile Phase B: Acetonitrile. Flow rate: 1.5 mL/min. COLUMN: HALO-C18 (30×2.0) mm, 2.7μ. Rt (min): 1.321; Area %—95.255.

### Step-3:tert-butyl((1r,4r)-4-((2-(2,6-bis(benzyloxy)pyridin-3-yl)-1-oxoisoindolin-4-yl)amino)cyclohexyl)carbamate

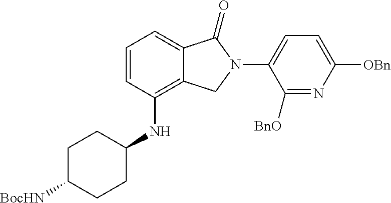 [SMILES: CC(C)(C)OC(=O)N[C@H]1CC[C@H](Nc2cccc3c2CN(c2ccc(OCc4ccccc4)nc2OCc2ccccc2)C3=O)CC1]

To a stirred mixture of tert-butyl N-[(1r,4r)-4-aminocyclohexyl]carbamate 1 (1 g, 4.666 mmol, 1 equiv), 2-[2,6-bis(benzyloxy)pyridin-3-yl]-4-bromo-3H-isoindol-1-one 2 (2.57 g, 5.133 mmol, 1.1 equiv), Pd₂(dba)₃·CHCl₃ (0.48 g, 0.467 mmol, 0.1 equiv), CPhos (0.41 g, 0.933 mmol, 0.2 equiv) and Cs₂CO₃ (4.56 g, 13.998 mmol, 3 equiv) in dioxane (20 mL). The resulting mixture was stirred for overnight at 100° C. under nitrogen atmosphere. The mixture was allowed to cool down to room temperature, filtered, and the filter cake was washed with dioxane (3×20 mL). The filtrate was concentrated under reduced pressure. The residue was purified by silica gel column chromatography, eluted with PE/EA (2:1) to afford tert-butyl((1r,4r)-4-((2-(2,6-bis(benzyloxy)pyridin-3-yl)-1-oxoisoindolin-4-yl)amino)cyclohexyl)carbamate 3 (1.9 g, 64.15%) as brown solid.

LCMS: 635.2 (M+H). Method: Mobile Phase A: Water/0.1% FA. Mobile Phase B: Acetonitrile/0.1% FA. Flow rate: 1.5 mL/min. COLUMN: XB-C18 (30×3.0) mm, 1.7μ. Rt (min): 1.273; Area %—94.64.

### Step-4:tert-butyl((1r,4r)-4-((2-(2,6-dioxopiperidin-3-yl)-1-oxoisoindolin-4-yl)amino)cyclohexyl)carbamate

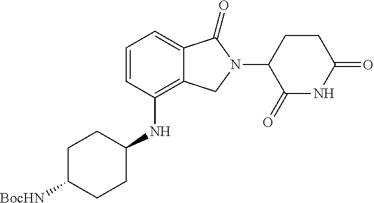 [SMILES: CC(C)(C)OC(=O)N[C@H]1CC[C@H](Nc2cccc3c2CN(C2CCC(=O)NC2=O)C3=O)CC1]

To a solution of tert-butyl((1r,4r)-4-((2-(2,6-bis(benzyloxy)pyridin-3-yl)-1-oxoisoindolin-4-yl)amino)cyclohexyl)carbamate 3 (1 g, 1.575 mmol, 1 equiv) in 10 mL DMF was added Pd/C (500 mg) in a pressure tank. The mixture was hydrogenated at room temperature under 5 psi of hydrogen pressure for overnight, filtered through a Celite pad and washed with DMF (2×3 mL). The resulting mixture was purified by reverse flash chromatography with the following conditions (column, C18 silica gel; mobile phase, MeCN in water (0.1% FA), 0% to 100% gradient in 10 min; detector, UV 254 nm) to afford tert-butyl((1r,4r)-4-((2-(2,6-dioxopiperidin-3-yl)-1-oxoisoindolin-4-yl)amino)cyclohexyl)carbamate 4 (590 mg, 82.03%) as pink solid.

LCMS: 457.1 (M+H). Method: Mobile Phase A: Water/0.1% FA. Mobile Phase B: Acetonitrile/0.1% FA. Flow rate: 1.5 mL/min. COLUMN: XB-C18 (30×3.0) mm, 1.7μ. Rt (min): 0.852; Area %—99.56.

### Step-5: tert-butyl ((1r,4r)-4-((2-(2,6-dioxopiperidin-3-yl)-1-oxoisoindolin-4-yl)(4,4,4-trifluorobutyl)amino)cyclohexyl)carbamate

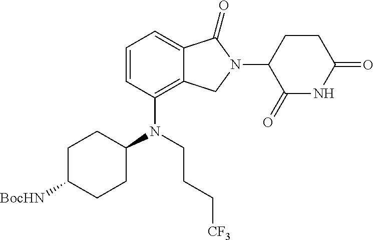 [SMILES: CC(C)(C)OC(=O)N[C@H]1CC[C@H](N(CCCC(F)(F)F)c2cccc3c2CN(C2CCC(=O)NC2=O)C3=O)CC1]

A solution of tert-butyl((1r,4r)-4-((2-(2,6-dioxopiperidin-3-yl)-1-oxoisoindolin-4-yl)amino)cyclohexyl)carbamate 4 (570 mg, 1.249 mmol, 1 equiv), 4,4,4-trifluorobutanal 5 (787.05 mg, 6.245 mmol, 5 equiv), TFA (427.08 mg, 3.747 mmol, 3 equiv) in DCM (14 mL) and DMF (3.5 mL) was stirred for 2 h at room temperature under nitrogen atmosphere followed by the addition of STAB (1323.05 mg, 6.245 mmol, 5 equiv) dropwise at 0° C. The resulting mixture was stirred overnight at room temperature under nitrogen atmosphere. The reaction was quenched with water at 0° C., and extracted with CH₂Cl₂ (3×50 mL). The combined organic layers were washed with brine (2×30 mL), dried over anhydrous Na₂SO₄, filtered and concentrated under reduced pressure. The residue was purified by silica gel column chromatography, eluted with PE/EA (2:1) to afford tert-butyl ((1r,4r)-4-((2-(2,6-dioxopiperidin-3-yl)-1-oxoisoindolin-4-yl)(4,4,4-trifluorobutyl)amino)cyclohexyl)carbamate 6 (700 mg, 98.95%) as off-white solid.

LCMS: 567.2 (M+H). Method: Mobile Phase A: Mobile Phase A: Water/5 mM NH₄HCO₃. Mobile Phase B: Acetonitrile. Flow rate: 1.5 mL/min. COLUMN: HPH-C18 (50×3.0) mm, 2.7μ. Rt (min): 0.895; Area %—97.38.

### Step-6: 3-(4-(((1r,4r)-4-aminocyclohexyl)(4,4,4-trifluorobutyl)amino)-1-oxoisoindolin-2-yl)piperidine-2,6-dione

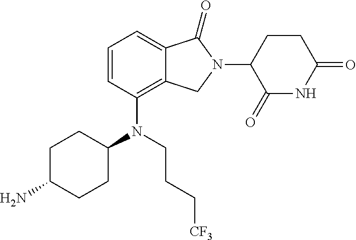 [SMILES: N[C@H]1CC[C@H](N(CCCC(F)(F)F)c2cccc3c2CN(C2CCC(=O)NC2=O)C3=O)CC1]

To a stirred solution of tert-butyl ((1r,4r)-4-((2-(2,6-dioxopiperidin-3-yl)-1-oxoisoindolin-4-yl)(4,4,4-trifluorobutyl)amino)cyclohexyl)carbamate 6 (650 mg, 1.147 mmol, 1 equiv) in DCM (10 mL) was added TFA (2 mL, 26.926 mmol, 23.47 equiv) dropwise at 0° C. under nitrogen atmosphere. The resulting mixture was stirred overnight at room temperature under nitrogen atmosphere, and concentrated under vacuum. This resulted in 3-(4-(((1r,4r)-4-aminocyclohexyl)(4,4,4-trifluorobutyl)amino)-1-oxoisoindolin-2-yl)piperidine-2,6-dione 7 (900 mg, crude) as brown yellow oil.

LCMS: 467.1 (M+H). Method: Mobile Phase A: Water/0.1% FA. Mobile Phase B: Acetonitrile/0.1% FA. Flow rate: 1.5 mL/min. COLUMN: XB-C18 (30×3.0) mm, 1.7μ. Rt (min): 0.587; Area %—100.0

### Step-7: 3-(4-(((1r,4r)-4-((3,3-difluorocyclobutyl)amino)cyclohexyl)(4,4,4-trifluorobutyl)amino)-1-oxoisoindolin-2-yl)piperidine-2,6-dione; formic acid

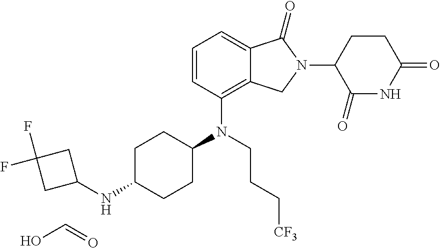

A solution of 3-(4-(((1r,4r)-4-aminocyclohexyl)(4,4,4-trifluorobutyl)amino)-1-oxoisoindolin-2-yl)piperidine-2,6-dione 7 (200 mg, 0.353 mmol, 1 equiv), 3,3-difluorocyclobutan-1-one 8 (187.20 mg, 1.765 mmol, 5 equiv), and TFA (120.74 mg, 1.059 mmol, 3 equiv) in DCM (5 mL) and DMF (1.2 mL) was stirred for 2 h at room temperature under nitrogen atmosphere followed by the addition of STAB (374.04 mg, 1.765 mmol, 5 equiv) dropwise at 0° C. The resulting mixture was stirred overnight at room temperature under nitrogen atmosphere. The resulting mixture was concentrated under vacuum. The residue was purified by prep-HPLC with the following conditions: Column: Sunfire prep C18 column, 30×150 mm, 5 μm; Mobile Phase A: Water (0.1% TFA), Mobile Phase B: ACN; Flow rate: 60 mL/min; Gradient: 20% B to 32% B in 9 min, 32% B; Wave Length: 254/220 nm; RT1 (min): 8.43. This resulted in 3-(4-(((1r,4r)-4-((3,3-difluorocyclobutyl)amino)cyclohexyl)(4,4,4-trifluorobutyl)amino)-1-oxoisoindolin-2-yl)piperidine-2,6-dione; formic acid (17.3 mg, 8.13%) as off-white solid.

¹H-NMR (400 MHz, Methanol-d₄): δ 10.98 (s, 1H), 9.08 (d, J=7.7 Hz, 2H), 7.46 (t, J=7.6 Hz, 1H), 7.37 (t, J=6.9 Hz, 2H), 5.12 (dd, J=13.2, 5.1 Hz, 1H), 4.36 (d, J=17.4 Hz, 1H), 4.24 (d, J=17.3 Hz, 1H), 3.81 (s, 1H), 3.18 (t, J=7.3 Hz, 2H), 3.08-2.78 (m, 7H), 2.69-2.52 (m, 2H), 2.22 (td, J=10.5, 6.5 Hz, 2H), 2.00 (ddd, J=21.5, 12.5, 8.8 Hz, 3H), 1.82 (d, J=9.5 Hz, 2H), 1.57-1.22 (m, 6H). LCMS: 557.2 (M+H). Method: Mobile Phase A: Water/5 mM NH₄HCO₃. Mobile Phase B: Acetonitrile. Flow rate: 1.5 mL/min. COLUMN: Kinetex EVO C18 (50×3.0) mm, 2.7μ. Rt (min): 1.700; Area %—95.08.

### Compound 513

### 3-(1-oxo-4-((4,4,4-trifluorobutyl)((1r,4r)-4-((3,3,3-trifluoropropyl)amino)cyclohexyl)amino)isoindolin-2-yl)piperidine-2,6-dione

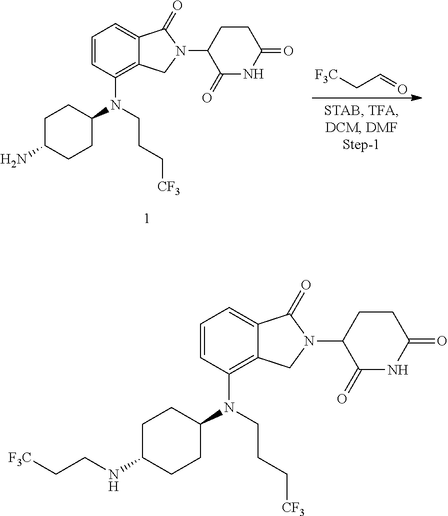 [SMILES: N[C@H]1CC[C@H](N(CCCC(F)(F)F)c2cccc3c2CN(C2CCC(=O)NC2=O)C3=O)CC1 ; O=CCC(F)(F)F ; O=C1CCC(N2Cc3c(cccc3N(CCCC(F)(F)F)[C@H]3CC[C@H](NCCC(F)(F)F)CC3)C2=O)C(=O)N1]

### Step-1: 3-(1-oxo-4-((4,4,4-trifluorobutyl)((1r,4r)-4-((3,3,3-trifluoropropyl)amino)cyclohexyl)amino)isoindolin-2-yl)piperidine-2,6-dione

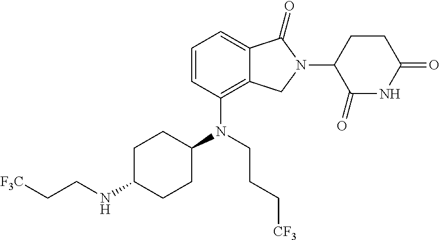 [SMILES: O=C1CCC(N2Cc3c(cccc3N(CCCC(F)(F)F)[C@H]3CC[C@H](NCCC(F)(F)F)CC3)C2=O)C(=O)N1]

A solution of 3-(4-(((1r,4r)-4-aminocyclohexyl)(4,4,4-trifluorobutyl)amino)-1-oxoisoindolin-2-yl)piperidine-2,6-dione 1 (200 mg, 0.353 mmol, 1 equiv), 3,3,3-trifluoropropanal (197.75 mg, 1.765 mmol, 5 equiv), TFA (120.74 mg, 1.059 mmol, 3 equiv) in DCM (5 mL) and DMF (1.2 mL) was stirred for 2 h at room temperature under nitrogen atmosphere followed by the addition of STAB (374.04 mg, 1.765 mmol, 5 equiv) dropwise at 0° C. The resulting mixture was stirred overnight at room temperature under nitrogen atmosphere. The resulting mixture was concentrated under vacuum. The residue was purified by prep-HPLC with the following conditions: Column: YMC-Actus Triart C18, 30×150 mm, 5 μm; Mobile Phase A: Water (0.1% FA), Mobile Phase B: ACN; Flow rate: 60 mL/min; Gradient: 15% B to 45% B in 7 min, 45% B; Wave Length: 254/220 nm; RT1 (min): 6.17. This resulted in 3-(1-oxo-4-((4,4,4-trifluorobutyl)((1r,4r)-4-((3,3,3-trifluoropropyl)amino)cyclohexyl)amino)isoindolin-2-yl)piperidine-2,6-dione (12 mg, 6.04%) as off-white solid.

¹H-NMR (400 MHz, Methanol-d₄): δ 7.54-7.38 (m, 3H), 5.14 (dd, J=13.3, 5.2 Hz, 1H), 4.44 (s, 2H), 3.24 (p, J=6.7 Hz, 2H), 3.06 (d, J=10.6 Hz, 3H), 2.91 (ddd, J=18.5, 13.4, 5.4 Hz, 1H), 2.84-2.72 (m, 2H), 2.64-2.42 (m, 3H), 2.24-2.03 (m, 5H), 1.95 (d, J=12.9 Hz, 2H), 1.55 (p, J=7.2 Hz, 4H), 1.31 (dt, J=24.8, 12.7 Hz, 3H). LCMS: 563.25 (M+H). Method: Mobile Phase A: Water/5 mM NH₄HCO₃. Mobile Phase B: Acetonitrile. Flow rate: 1.5 mL/min. COLUMN: Kinetex EVO C18 (50×3.0) mm, 2.7μ. Rt (min): 1.727; Area %—96.85.

### Compound 514

### 3-(1-oxo-4-{[(1r,4r)-4-(oxetan-3-ylamino)cyclohexyl](4,4,4-trifluorobutyl)amino}-3H-isoindol-2-yl)piperidine-2,6-dione

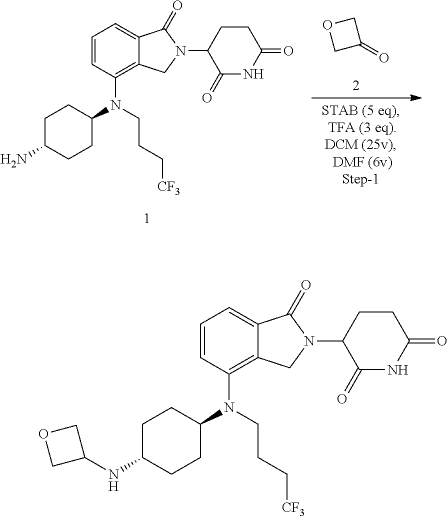 [SMILES: N[C@H]1CC[C@H](N(CCCC(F)(F)F)c2cccc3c2CN(C2CCC(=O)NC2=O)C3=O)CC1 ; O=C1COC1 ; O=C1CCC(N2Cc3c(cccc3N(CCCC(F)(F)F)[C@H]3CC[C@H](NC4COC4)CC3)C2=O)C(=O)N1]

### Step-1: 3-(1-oxo-4-{[(1r,4r)-4-(oxetan-3-ylamino)cyclohexyl](4,4,4-trifluorobutyl)amino}-3H-isoindol-2-yl)piperidine-2,6-dione

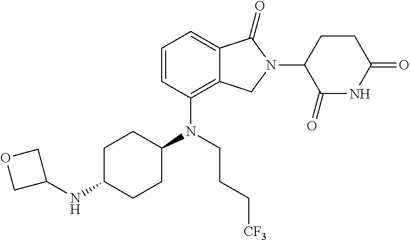 [SMILES: O=C1CCC(N2Cc3c(cccc3N(CCCC(F)(F)F)[C@H]3CC[C@H](NC4COC4)CC3)C2=O)C(=O)N1]

A solution of 3-(1-oxo-4-{[(1r,4r)-4-aminocyclohexyl](4,4,4-trifluorobutyl)amino}-3H-isoindol-2-yl)piperidine-2,6-dione 1 (360 mg, 0.772 mmol, 1 equiv) and 3-oxetanone 2 (278.05 mg, 3.860 mmol, 5 equiv) in DCM (9.00 mL, 141.574 mmol) and DMF (2.6 mL, 33.595 mmol) was treated with TFA (263.97 mg, 2.316 mmol, 3 equiv) at 0 degrees C. under nitrogen atmosphere followed by the addition of STAB (817.77 mg, 3.860 mmol, 5 equiv) dropwise at 0° C. The resulting mixture was stirred for additional 12 h at room temperature. The resulting mixture was concentrated under reduced pressure. The residue was purified by reverse flash chromatography with the following conditions: column, C18 silica gel; mobile phase, MeCN in Water (0.1% FA), 10% to 50% gradient in 10 min; detector, UV 254 nm. This resulted in 3-(1-oxo-4-{[(1r,4r)-4-(oxetan-3-ylamino)cyclohexyl](4,4,4-trifluorobutyl)amino}-3H-isoindol-2-yl)piperidine-2,6-dione (37.2 mg, 96.79%) as white solid.

¹H-NMR (300 MHz, Methanol-d₄): δ 7.61-7.47 (m, 3H), 7.47 (dd, J=7.2, 2.0 Hz, 1H), 5.16 (dd, J=13.2, 5.2 Hz, 2H), 4.91 (t, J=7.4 Hz, 2H), 4.68 (dd, J=7.8, 5.5 Hz, 1H), 4.55 (p, J=6.3 Hz, 2H), 4.47 (s, 2H), 3.24 (dq, J=13.4, 6.6 Hz, 2H), 3.10 (s, 2H), 2.94 (ddd, J=18.3, 13.3, 5.3 Hz, 1H), 2.81 (ddd, J=17.5, 4.7, 2.4 Hz, 3H), 2.58 (qd, J=13.2, 4.8 Hz, 4H), 2.28-2.15 (m, 6H), 2.20-1.97 (m, OH). LCMS: 523.25 (M+H). Method: Mobile Phase A: Water/5 mM NH₄HCO₃. Mobile Phase B: Acetonitrile. Flow rate: 1.5 mL/min. COLUMN: Kinetex EVO C18 (50×3.0) mm, 2.7μ. Rt (min): 1.357; Area %—96.787.

### Compound 515

### 3-(4-((2-cyclopropylethyl)((1r,4r)-4-((3,3-difluorocyclobutyl)amino)cyclohexyl)amino)-1-oxoisoindolin-2-yl)piperidine-2,6-dione formate

### Compound 515A

### 3-(4-((2-cyclopropylethyl)((1s,4s)-4-((3,3-difluorocyclobutyl)amino)cyclohexyl)amino)-1-oxoisoindolin-2-yl)piperidine-2,6-dione formate

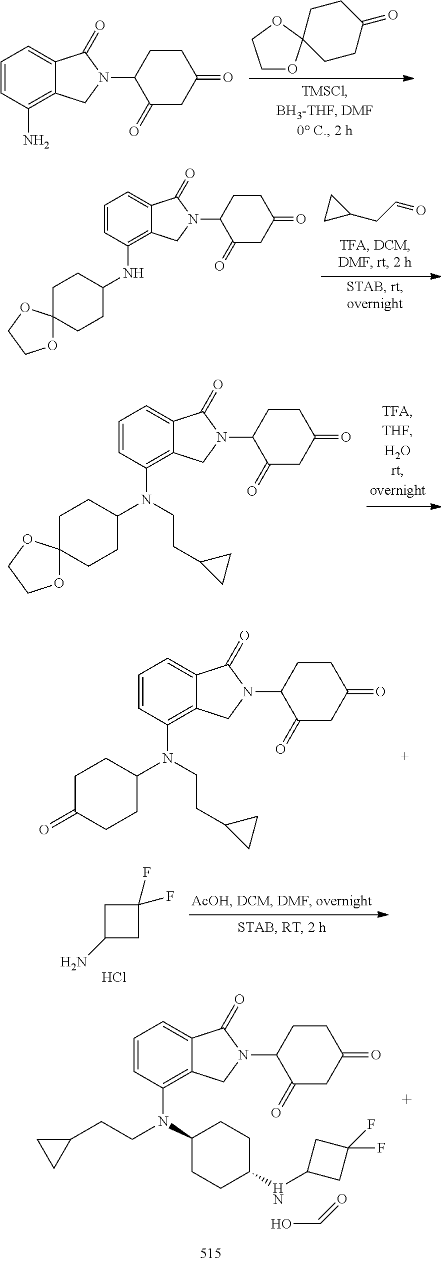

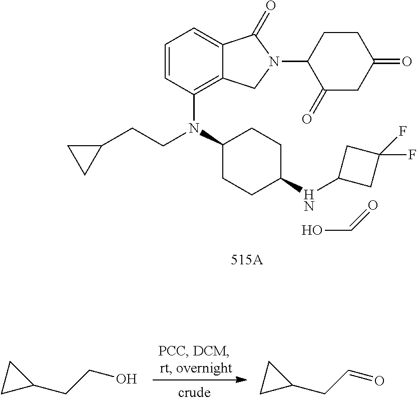

### Step-1: 2-cyclopropylacetaldehyde

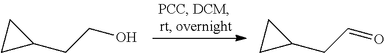 [SMILES: OCCC1CC1 ; O=CCC1CC1]

To a stirred solution of 2-cyclopropylethanol (4.0 g, 46.4 mmol, 1 equiv) in DCM (80.00 mL) was added PCC (15.0 g, 69.65 mmol, 1.5 equiv) in portions at 0° C. under nitrogen atmosphere. The resulting mixture was stirred for overnight at room temperature under nitrogen atmosphere. The resulting mixture was filtered, the filter cake was washed with DCM (3×10 mL). The filtrate was concentrated under reduced pressure at 0° C. The crude product, 2-cyclopropylacetaldehyde, was used in the next step directly without further purification.

### Step-2: 3-(4-{1,4-dioxaspiro[4.5]decan-8-ylamino}-1-oxo-3H-isoindol-2-yl)piperidine-2,6-dione

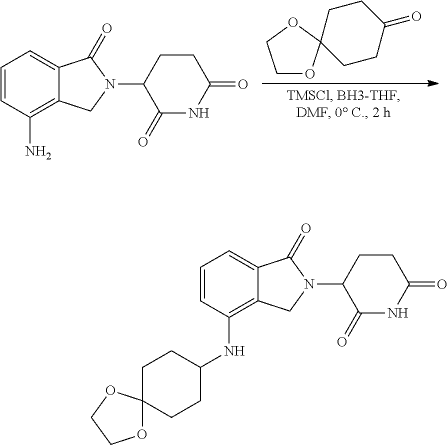

To a stirred solution of lenalidomide (3.0 g, 11.57 mmol, 1 equiv) and 1,4-dioxaspiro[4.5]decan-8-one (9.0 g, 57.8 mmol, 5 equiv) in DMF (60 mL) was added TMSCl (14.8 mL, 115 mmol, 10 equiv) dropwise at 0° C. under nitrogen atmosphere. The resulting mixture was stirred for 1 h at 0° C. under nitrogen atmosphere. To the above mixture was added BH3-THF (34.7 mL, 34.7 mmol, 3 equiv) dropwise over 15 min at 0° C. The resulting mixture was stirred for additional 1 h at 0° C. The reaction was quenched by the addition of sat. NH₄C₁ (aq.) (300 mL) at 0° C. The resulting mixture was extracted with EtOAc (3×250 mL). The combined organic layers were washed with brine (3×400 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 0% to 100% gradient in 10 min; detector, UV 254 nm to afford 3-(4-{1,4-dioxaspiro[4.5]decan-8-ylamino}-1-oxo-3H-isoindol-2-yl)piperidine-2,6-dione (2.2 g, 47%) as a light yellow solid.

### Step-3: 3-{4-[(2-cyclopropylethyl)(1,4-dioxaspiro[4.5]decan-8-yl)amino]-1-oxo-3H-isoindol-2-yl}piperidine-2,6-dione

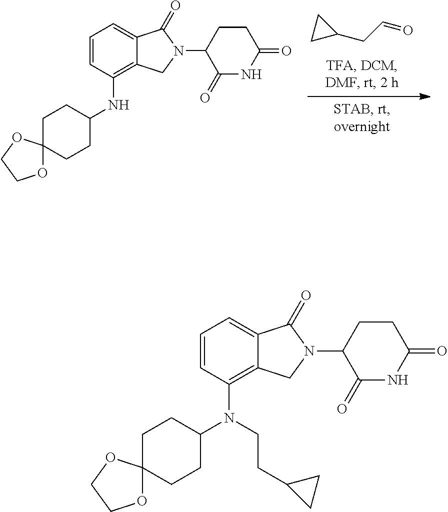

To a stirred solution of 3-(4-{1,4-dioxaspiro[4.5]decan-8-ylamino}-1-oxo-3H-isoindol-2-yl)piperidine-2,6-dione (300 mg, 0.75 mmol, 1 equiv) and 2-cyclopropylacetaldehyde (1263 mg, 15 mmol, 20 equiv) in DCM (3.6 mL) and DMF (1 mL) was added TFA (257 mg, 2.25 mmol, 3 equiv) at room temperature under nitrogen atmosphere. The resulting mixture was stirred for 2 h at room temperature under nitrogen atmosphere. To the above mixture was added STAB (796 mg, 3.75 mmol, 5 equiv) in portions over 2 min at 0° C. The resulting mixture was stirred for additional overnight at room temperature. The resulting mixture was diluted with water at room temperature. The resulting mixture was extracted with EtOAc (3×100 mL). The combined organic layers were washed with brine (3×300 mL), dried over anhydrous Na₂SO₄. After filtration, the filtrate was concentrated under reduced pressure. The residue was purified by reverse flash chromatography with the following conditions: column, C18 silica gel; mobile phase, MeCN in Water (0.1% FA), 0% to 100% gradient in 15 min; detector, UV 254 nm to afford 3-{4-[(2-cyclopropylethyl)(1,4-dioxaspiro[4.5]decan-8-yl)amino]-1-oxo-3H-isoindol-2-yl}piperidine-2,6-dione (200 mg, 57%) as a light yellow oil.

### Step-4: 3-{4-[(2-cyclopropylethyl)(4-oxocyclohexyl)amino]-1-oxo-3H-isoindol-2-yl}piperidine-2,6-dione

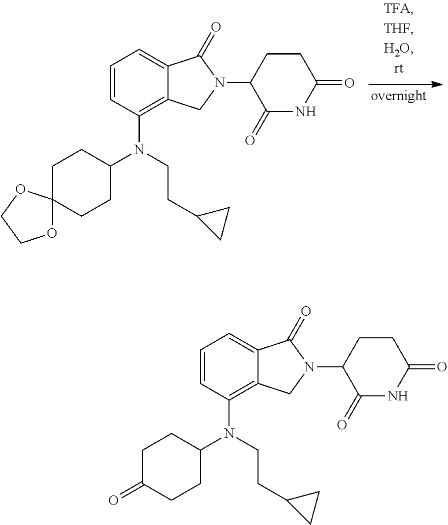 [SMILES: O=C1CCC(N2Cc3c(cccc3N(CCC3CC3)C3CCC4(CC3)OCCO4)C2=O)C(=O)N1 ; O=C1CCC(N(CCC2CC2)c2cccc3c2CN(C2CCC(=O)NC2=O)C3=O)CC1]

To a stirred solution of 3-{4-[(2-cyclopropylethyl)(1,4-dioxaspiro[4.5]decan-8-yl)amino]-1-oxo-3H-isoindol-2-yl}piperidine-2,6-dione (450 mg, 0.96 mmol, 1 equiv) in THF (4.50 mL) and H₂O (4.50 mL) was added TFA (4.5 mL, 60.5 mmol, 63 equiv) dropwise at room temperature under nitrogen atmosphere. The resulting mixture was stirred for overnight at room temperature. The resulting mixture was concentrated under reduced pressure. The residue was purified by reverse flash chromatography with the following conditions: column, C18 silica gel; mobile phase, MeCN in Water (0.1% FA), 0% to 100% gradient in 12 min; detector, UV 254 nm to afford 3-{4-[(2-cyclopropylethyl)(4-oxocyclohexyl)amino]-1-oxo-3H-isoindol-2-yl}piperidine-2,6-dione (360 mg, 88%) as a yellow oil.

### Step-5: 3-(4-((2-cyclopropylethyl)((1r,4r)-4-((3,3-difluorocyclobutyl)amino)cyclohexyl)amino)-1-oxoisoindolin-2-yl)piperidine-2,6-dione formate and 3-(4-((2-cyclopropylethyl)((1s,4s)-4-((3,3-difluorocyclobutyl)amino)cyclohexyl)amino)-1-oxoisoindolin-2-yl)piperidine-2,6-dione formate

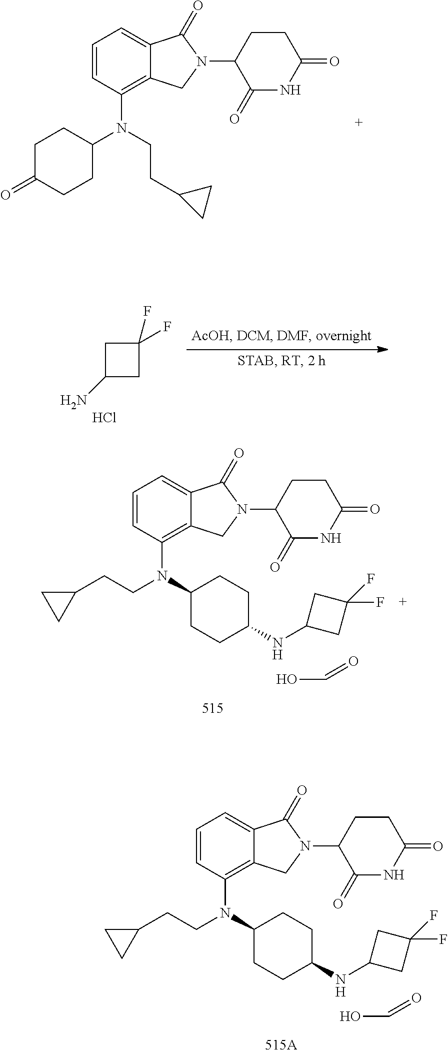

To a stirred solution of 3-{4-[(2-cyclopropylethyl)(4-oxocyclohexyl)amino]-1-oxo-3H-isoindol-2-yl}piperidine-2,6-dione (190 mg, 0.45 mmol, 1 equiv) and 3,3-difluorocyclobutan-1-amine hydrochloride (322 mg, 2.24 mmol, 5 equiv) in DCM (5 mL, 78.6 mmol, 175 equiv) and DMF (1.2 mL) was added AcOH (0.08 mL, 1.34 mmol, 3 equiv) at room temperature under nitrogen atmosphere. The resulting mixture was stirred for overnight at room temperature under nitrogen atmosphere. To the above mixture was added STAB (380 mg, 1.79 mmol, 4 equiv) in portions over 2 min at 0° C. The resulting mixture was stirred for additional 2 h at room temperature. The resulting mixture was diluted with water (50 mL) and extracted with EtOAc (3×50 mL). The combined organic layers were washed with brine (3×50 mL), dried over anhydrous Na₂SO₄. After filtration, the filtrate was concentrated under reduced pressure. The crude product (100 mg) was purified by Prep-HPLC with the following conditions (Column: Xcelect CSH F-pheny OBD Column, 19*250 mm, 5 μm; Mobile Phase A: Water (0.1% FA), Mobile Phase B: ACN; Flow rate: 25 mL/min; Gradient: 20% B to 45% B in 8 min; Wave Length: 254/220 nm; RT1 (min): 5.93) to afford 3-(4-((2-cyclopropylethyl)((1r,4r)-4-((3,3-difluorocyclobutyl)amino)cyclohexyl)amino)-1-oxoisoindolin-2-yl)piperidine-2,6-dione formate (11.5 mg, 5%) as an off-white solid and 3-(4-((2-cyclopropylethyl)((1s,4s)-4-((3,3-difluorocyclobutyl)amino)cyclohexyl)amino)-1-oxoisoindolin-2-yl)piperidine-2,6-dione formate (23.6 mg, 10%) as off-white solid.

Compound 515: LCMS: 515.35 (M+H). ¹H NMR (400 MHz, DMSO-d6):δ 11.03 (s, 1H), 7.49 (t, J=7.6 Hz, 1H), 7.35 (dd, J=16.5, 7.6 Hz, 2H), 5.17 (dt, J=13.4, 4.9 Hz, 1H), 4.51-4.23 (m, 2H), 3.63 (d, J=39.6 Hz, 2H), 3.35-3.22 (m, 2H), 3.21-3.10 (m, 1H), 3.08-2.85 (m, 4H), 2.69 (s, 4H), 2.07 (dt, J=17.0, 8.5 Hz, 3H), 1.88 (d, J=11.1 Hz, 2H), 1.62 (t, J=10.5 Hz, 2H), 1.33 (t, J=10.2 Hz, 2H), 1.22 (p, J=7.2 Hz, 2H), 0.70 (q, J=6.7 Hz, 1H), 0.40 (dq, J=8.1, 4.2 Hz, 2H), −0.00 (t, J=4.7 Hz, 2H).

Compound 515A: LCMS: 515.3 (M+H). ¹H NMR (400 MHz, DMSO-d6):δ 11.03 (s, 1H), 7.48 (t, J=7.6 Hz, 1H), 7.35 (dd, J=16.1, 7.6 Hz, 2H), 5.16 (dd, J=13.2, 5.1 Hz, 1H), 4.52-4.26 (m, 3H), 3.34-3.12 (m, 4H), 2.90 (ddt, J=14.1, 6.7, 4.1 Hz, 4H), 2.71-2.59 (m, 3H), 2.17-2.01 (m, 1H), 1.82 (d, J=43.9 Hz, 4H), 1.66-1.47 (m, 4H), 1.23 (q, J=7.2 Hz, 2H), 0.69 (ddt, J=10.1, 7.2, 4.1 Hz, 1H), 0.48-0.32 (m, 2H), −0.00 (td, J=5.4, 3.9 Hz, 2H).

### Compound 516

### 3-{4-[(2-cyclopropylethyl)[(1r,4r)-4-[(3,3,3-trifluoropropyl)amino]cyclohexyl]amino]-1-oxo-3H-isoindol-2-yl}piperidine-2,6-dione

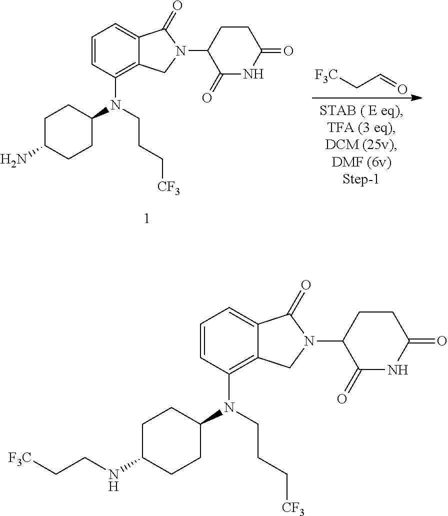 [SMILES: N[C@H]1CC[C@H](N(CCCC(F)(F)F)c2cccc3c2CN(C2CCC(=O)NC2=O)C3=O)CC1 ; O=CCC(F)(F)F ; O=C1CCC(N2Cc3c(cccc3N(CCCC(F)(F)F)[C@H]3CC[C@H](NCCC(F)(F)F)CC3)C2=O)C(=O)N1]

### Step-1: 3-{4-[(2-cyclopropylethyl)[(1r,4r)-4-[(3,3,3-trifluoropropyl)amino]cyclohexyl]amino]-1-oxo-3H-isoindol-2-yl}piperidine-2,6-dione

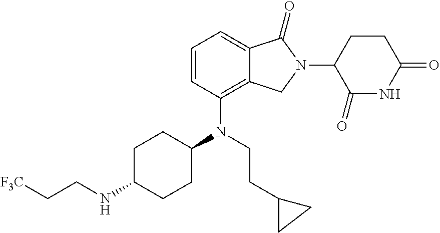 [SMILES: O=C1CCC(N2Cc3c(cccc3N(CCC3CC3)[C@H]3CC[C@H](NCCC(F)(F)F)CC3)C2=O)C(=O)N1]

A solution of 3-{4-[(2-cyclopropylethyl)[(1r,4r)-4-aminocyclohexyl]amino]-1-oxo-3H-isoindol-2-yl}piperidine-2,6-dione 1 (Compound 237, 110 mg, 0.259 mmol, 1 equiv) and 3,3,3-trifluoropropanal 2 (72.58 mg, 0.647 mmol, 2.5 equiv) in DCM (2.75 mL, 43.259 mmol) and DMF (0.66 mL, 8.528 mmol) was treated with TFA (88.63 mg, 0.777 mmol, 3 equiv) at 0 degrees C. under nitrogen atmosphere followed by the addition of STAB (274.57 mg, 1.295 mmol, 5 equiv) dropwise at 0 degrees C. The resulting mixture was stirred for additional 12 h at room temperature. The resulting mixture was concentrated under reduced pressure. The residue was purified by reverse flash chromatography with the following conditions: column, C18 silica gel; mobile phase, MeCN in Water (0.1% FA), 10% to 50% gradient in 10 min; detector, UV 254 nm. Desired product could be detected by LCMS. This resulted in 3-{4-[(2-cyclopropylethyl)[(1r,4r)-4-[(3,3,3-trifluoropropyl)amino]cyclohexyl]amino]-1-oxo-3H-isoindol-2-yl}piperidine-2,6-dione (3.5 mg, 96.14%) as white solid.

¹H-NMR (400 MHz, DMSO-d₆): δ 7.42 (t, J=7.7 Hz, 1H), 7.29 (d, J=7.3 Hz, 1H), 7.24 (d, J=8.0 Hz, 1H), 5.07 (dd, J=13.4, 5.1 Hz, 1H), 4.35 (d, J=17.2 Hz, 2H), 4.26 (d, J=17.1 Hz, 2H), 3.20 (s, 1H), 3.07 (s, 1H), 2.94-2.81 (m, 2H), 2.72 (t, J=8.2 Hz, 2H), 2.61 (d, J=17.2 Hz, 4H), 2.36 (s, 2H), 2.03 (d, J=13.2 Hz, 2H), 1.89 (d, J=12.3 Hz, 2H), 1.74 (d, J=12.3 Hz, 2H), 1.52 (s, OH), 1.24 (s, 4H), 1.17-1.09 (m, OH), 1.06 (d, J=12.5 Hz, 1H), 0.33 (d, J=7.8 Hz, 2H), −0.07 (d, J=4.9 Hz, 2H). LCMS: 521.27 (M+H). Method: Mobile Phase A: Water/5 mM NH₄HCO₃. Mobile Phase B: Acetonitrile. Flow rate: 1.5 mL/min. COLUMN: Kinetex EVO C18 (50×3.0) mm, 2.7μ. Rt (min): 1.300; Area %—96.14.

### Compound 517

### 3-(4-((2-cyclopropylethyl)((1r,4r)-4-(oxetan-3-ylamino)cyclohexyl)amino)-1-oxoisoindolin-2-yl)piperidine-2,6-dione

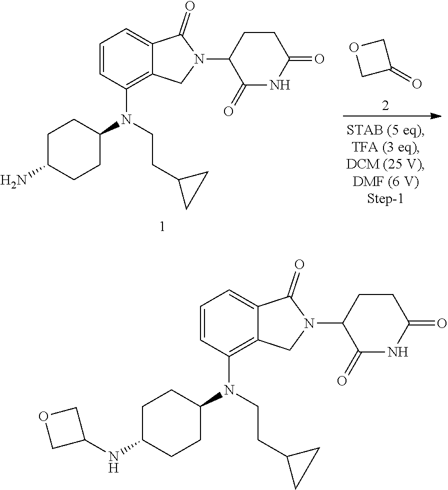

### Step-1:3-(4-((2-cyclopropylethyl)((1r,4r)-4-(oxetan-3-ylamino)cyclohexyl)amino)-1-oxoisoindolin-2-yl)piperidine-2,6-dione

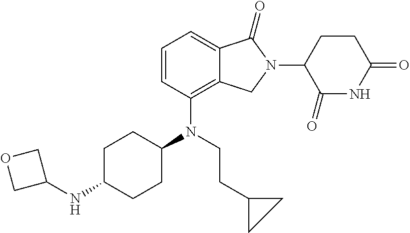 [SMILES: O=C1CCC(N2Cc3c(cccc3N(CCC3CC3)[C@H]3CC[C@H](NC4COC4)CC3)C2=O)C(=O)N1]

To a stirred solution of 3-{4-[(2-cyclopropylethyl)[(1r,4r)-4-aminocyclohexyl]amino]-1-oxo-3H-isoindol-2-yl}piperidine-2,6-dione 1 (200 mg, 0.471 mmol, 1 equiv) and 3-oxetanone 2 (169.74 mg, 2.355 mmol, 5 equiv) in DCM (5.00 mL, 78.653 mmol, 166.96 equiv) and DMF (1.20 mL, 15.501 mmol, 32.91 equiv) was added TFA (161.15 mg, 1.413 mmol, 3 equiv) dropwise at room temperature under nitrogen atmosphere. The resulting mixture was stirred for 2 h at room temperature under nitrogen atmosphere. To the above mixture was added STAB (499.22 mg, 2.355 mmol, 5 equiv) in portions over 2 min at 0° C. The resulting mixture was stirred for additional overnight at room temperature. The resulting mixture was extracted with EtOAc (3×100 mL). The combined organic layers were washed with brine (3×100 mL), dried over Na₂SO₄, filtered and concentrated. The crude product (80 mg) was purified by Prep-HPLC with the following conditions (Column: XBridge Shield RP18 OBD Column, 30*150 mm, 5 μm; Mobile Phase A: Water (0.1% FA), Mobile Phase B: ACN; Flow rate: 60 mL/min; Gradient: 5% B to 30% B in 8 min, 30% B; Wave Length: 254/220 nm; RT1 (min): 7.03; Number Of Runs: 0) to afford 3-{4-[(2-cyclopropylethyl)[(1r,4r)-4-(oxetan-3-ylamino)cyclohexyl]amino]-1-oxo-3H-isoindol-2-yl}piperidine-2,6-dione (13 mg, 5.74%) as off-white solid.

¹H-NMR (300 MHz, DMSO-d₆): δ 10.97 (s, 1H), 8.16 (s, 1H), 7.41 (t, J=7.6 Hz, 1H), 7.25 (dd, J=15.1, 7.6 Hz, 2H), 5.10 (dd, J=13.2, 5.1 Hz, 1H), 4.62 (t, J=6.6 Hz, 2H), 4.47-4.10 (m, 4H), 3.98 (p, J=6.8 Hz, 1H), 3.19 (t, J=7.1 Hz, 2H), 3.08 (d, J=11.4 Hz, 1H), 2.91 (s, 1H), 2.56 (s, 1H), 2.46-2.26 (m, 1H), 2.01 (dd, J=8.5, 3.8 Hz, 1H), 1.89-1.63 (m, 4H), 1.50 (d, J=12.6 Hz, 2H), 1.29-0.96 (m, 4H), 0.63 (s, 1H), 0.42-0.24 (m, 2H), −0.07 (dd, J=4.9, 1.5 Hz, 2H).LCMS:481.3 (M+H). Method: Mobile Phase A: 0.04% NH₃H₂O in H₂O. Mobile Phase B: Acetonitrile. Flow rate: 1.5 mL/min. COLUMN: EVO-C18 (50×3.0) mm, 2.6μ. Rt (min): 1.658; Area %—96.34.

### Compound 518

### 3-(4-((4-amino-3,3-difluorobutyl)(2-cyclopropylethyl)amino)-1-oxoisoindolin-2-yl)piperidine-2,6-dione

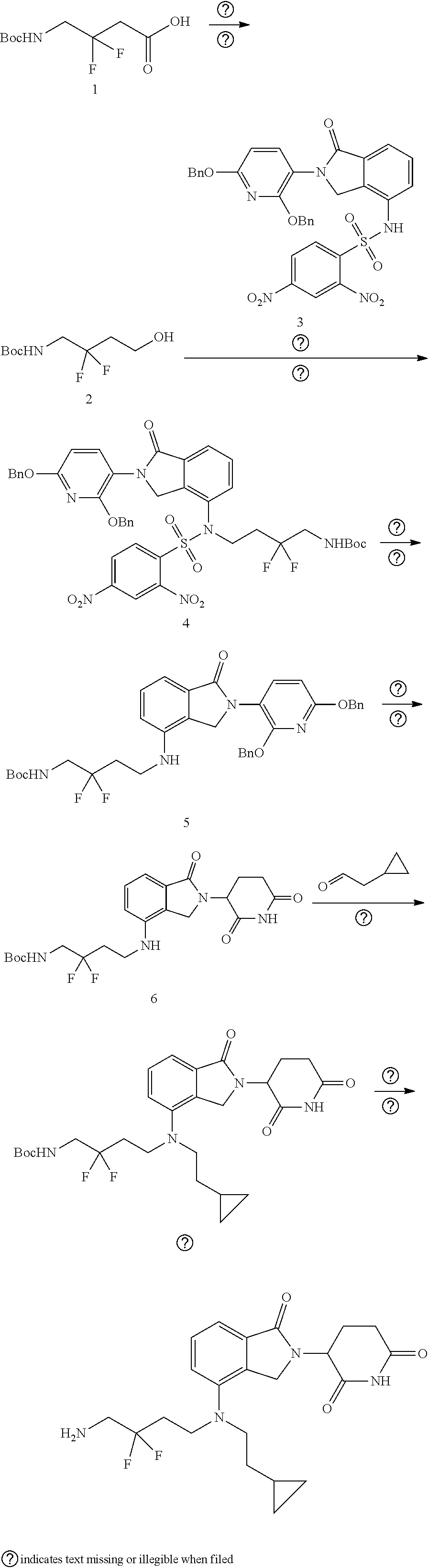

### Step-1: tert-butyl (2,2-difluoro-4-hydroxybutyl)carbamate

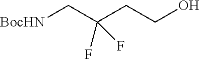 [SMILES: CC(C)(C)OC(=O)NCC(F)(F)CCO]

To solution of 4-[(tert-butoxycarbonyl)amino]-3,3-difluorobutanoic acid 1 (500 mg, 2.090 mmol, 1 equiv) in THF (10 mL, 123.428 mmol, 59.05 equiv) was added LiAlH₄ (1.67 mL, 4.180 mmol, 2 equiv) dropwise at room temperature under nitrogen atmosphere. The resulting mixture was stirred for overnight at room temperature under nitrogen atmosphere. The resulting mixture was extracted with EtOAc (3×50 mL). The combined organic layers were washed with brine (3×50 mL), dried over anhydrous Na₂SO₄, filtered and concentrated. The crude product was used in the next step directly without further purification.

LC-MS: 226.1 (M+H). Method: Mobile Phase A: 0.1% FA in H₂O. Mobile Phase B: Acetonitrile. Flow rate: 1.5 mL/min. COLUMN: HPHC18 (50×3.0) mm, 5μ. Rt (min): 0.756; Area %—85.30.

### Step-2: tert-butyl(4-((N-(2-(2,6-bis(benzyloxy)pyridin-3-yl)-1-oxoisoindolin-4-yl)-2,4-dinitrophenyl)sulfonamido)-2,2-difluorobutyl)carbamate

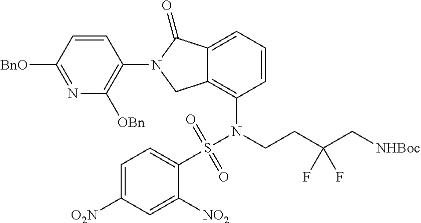 [SMILES: CC(C)(C)OC(=O)NCC(F)(F)CCN(c1cccc2c1CN(c1ccc(OCc3ccccc3)nc1OCc1ccccc1)C2=O)S(=O)(=O)c1ccc([N+](=O)[O-])cc1[N+](=O)[O-]]

To a stirred solution of N-{2-[2,6-bis(benzyloxy)pyridin-3-yl]-1-oxo-3H-isoindol-4-yl}-2,4-dinitrobenzenesulfonamide 3 (440 mg, 0.659 mmol, 1 equiv) and tert-butyl N-(2,2-difluoro-4-hydroxybutyl)carbamate 2 (445.31 mg, 1.977 mmol, 3 equiv) in THF (9 mL, 111.085 mmol, 168.56 equiv) were added PPh₃ (518.57 mg, 1.977 mmol, 3 equiv) and DIAD (399.79 mg, 1.977 mmol, 3 equiv) dropwise at 0° C. under nitrogen atmosphere. The resulting mixture was stirred for overnight at room temperature under nitrogen atmosphere. The resulting mixture was extracted with EtOAc (3×100 mL). The combined organic layers were washed with brine (3×100 mL), dried over anhydrous Na₂SO₄, filtered and concentrated. The residue was purified by reverse flash chromatography with the following conditions: column, C18 silica gel; mobile phase, MeCN in Water (0.1% FA), 0% to 100% gradient in 10 min; detector, UV 254 nm to afford tert-butyl N-[4-(N-{2-[2,6-bis(benzyloxy)pyridin-3-yl]-1-oxo-3H-isoindol-4-yl}2,4-dinitrobenzenesulfonamido)-2,2-difluorobutyl]carbamate 4 (260 mg, 45.09%) as yellow solid.

LC-MS: 875.2 (M+H). Method: Mobile Phase A: 0.1% FA in H₂O. Mobile Phase B: Acetonitrile. Flow rate: 1.5 mL/min. COLUMN: Halo C18 (30×5.0) mm, 5μ. Rt (min): 1.286; Area %—97.29.

### Step-3: tert-butyl (4-((2-(2,6-bis(benzyloxy)pyridin-3-yl)-1-oxoisoindolin-4-yl)amino)-2,2-difluorobutyl)carbamate

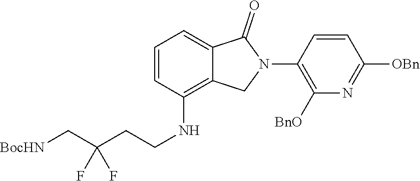 [SMILES: CC(C)(C)OC(=O)NCC(F)(F)CCNc1cccc2c1CN(c1ccc(OCc3ccccc3)nc1OCc1ccccc1)C2=O]

To a stirred solution of tert-butyl N-[4-(N-{2-[2,6-bis(benzyloxy)pyridin-3-yl]-1-oxo-3H-isoindol-4-yl}2,4-dinitrobenzenesulfonamido)-2,2-difluorobutyl]carbamate 4 (250 mg, 0.286 mmol, 1 equiv) and TEA (144.58 mg, 1.430 mmol, 5 equiv) in DMF (5.00 mL, 64.662 mmol, 226.09 equiv) was added thioglycolic acid (78.96 mg, 0.858 mmol, 3 equiv) dropwise at room temperature under nitrogen atmosphere. The resulting mixture was stirred for 2 h at room temperature under nitrogen atmosphere. The resulting mixture was extracted with EtOAc (3×100 mL). The combined organic layers were washed with brine (3×50 mL), dried over anhydrous Na₂SO₄, filtered and concentrated. The residue was purified by reverse flash chromatography with the following conditions: column, C18 silica gel; mobile phase, MeCN in Water (0.1% NH₃·H₂O), 0% to 100% gradient in 10 min; detector, UV 254 nm to afford tert-butyl N-[4-({2-[2,6-bis(benzyloxy)pyridin-3-yl]-1-oxo-3H-isoindol-4-yl}amino)-2,2-difluorobutyl]carbamate 5 (157 mg, 85.22%) as yellow oil.

LC-MS: 645.3 (M+H). Method: Mobile Phase A: 0.04% NH₃H₂O in H₂O. Mobile Phase B: Acetonitrile. Flow rate: 1.5 mL/min. COLUMN: EVO-C18 (50×3.0) mm, 5μ. Rt (min): 1.508; Area %—98.52.

### Step-4:tert-butyl(4-((2-(2,6-dioxopiperidin-3-yl)-1-oxoisoindolin-4-yl)amino)-2,2-difluorobutyl)carbamate

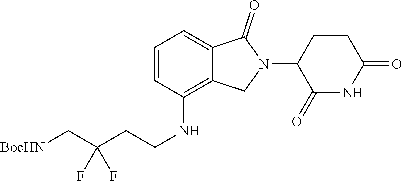 [SMILES: CC(C)(C)OC(=O)NCC(F)(F)CCNc1cccc2c1CN(C1CCC(=O)NC1=O)C2=O]

To a solution of tert-butyl N-[4-({2-[2,6-bis(benzyloxy)pyridin-3-yl]-1-oxo-3H-isoindol-4-yl}amino)-2,2-difluorobutyl]carbamate 5 (210 mg, 0.326 mmol, 1 equiv) in DMF (4.20 mL, 54.318 mmol, 166.62 equiv) was added Pd/C (335.89 mg, 3.159 mmol, 9.69 equiv) at room temperature under nitrogen atmosphere. The resulting mixture was stirred for overnight at room temperature under hydrogen atmosphere. The resulting mixture was filtered, the filter cake was washed with EtOAc (3×5 mL). The filtrate was extracted with EtOAc (3×50 mL). The combined organic layers were washed with brine (3×50 mL), dried over anhydrous Na₂SO₄, filtered and concentrated. The crude product was used in the next step directly without further purification.

LC-MS: 467.2 (M+H). Method: Mobile Phase A: 0.1% FA in H₂O. Mobile Phase B: Acetonitrile. Flow rate: 1.5 mL/min. COLUMN: HPHC18 (50×3.0) mm, 5μ. Rt (min): 0.879; Area %—98.69.

### Step-5:tert-butyl(4-((2-cyclopropylethyl)(2-(2,6-dioxopiperidin-3-yl)-1-oxoisoindolin-4-yl)amino)-2,2-difluorobutyl)carbamate

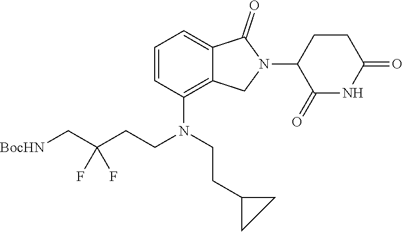 [SMILES: CC(C)(C)OC(=O)NCC(F)(F)CCN(CCC1CC1)c1cccc2c1CN(C1CCC(=O)NC1=O)C2=O]

To a stirred solution of tert-butyl N-(4-{[2-(2,6-dioxopiperidin-3-yl)-1-oxo-3H-isoindol-4-yl]amino}-2,2-difluorobutyl)carbamate 6 (170 mg, 0.364 mmol, 1 equiv) and 2-cyclopropylacetaldehyde (613.10 mg, 7.280 mmol, 20 equiv) in DCM (4.25 mL, 66.776 mmol, 183.45 equiv) and DMF (1.02 mL, 13.166 mmol, 36.17 equiv) was added TFA (124.66 mg, 1.092 mmol, 3 equiv) dropwise at room temperature under nitrogen atmosphere. The resulting mixture was stirred for 2 h at room temperature under nitrogen atmosphere. To the above mixture was added STAB (386.18 mg, 1.820 mmol, 5 equiv) in portions over 1 min at 0° C. The resulting mixture was stirred for additional overnight at room temperature. The resulting mixture was extracted with EtOAc (3 x100 mL). The combined organic layers were washed with brine (3×50 mL), dried over anhydrous Na₂SO₄, filtered and concentrated. The residue was purified by reverse flash chromatography with the following conditions: column, C18 silica gel; mobile phase, MeCN in Water (0.1% FA), 10% to 50% gradient in 10 min; detector, UV 254 nm to afford tert-butyl N-{4-[(2-cyclopropylethyl)[2-(2,6-dioxopiperidin-3-yl)-1-oxo-3H-isoindol-4-yl]amino]-2,2-difluorobutyl}carbamate 7 (110 mg, 56.46%) as off-white solid.

LC-MS: 535.3 (M+H). Method: Mobile Phase A: 0.05% TFA in H₂O. Mobile Phase B: Acetonitrile. Flow rate: 1.8 mL/min. COLUMN: Ascentis Express C18 (30×3.0) mm, 5μ. Rt (min): 1.246; Area %—97.55.

### Step-6:3-(4-((4-amino-3,3-difluorobutyl)(2-cyclopropylethyl)amino)-1-oxoisoindolin-2-yl)piperidine-2,6-dione

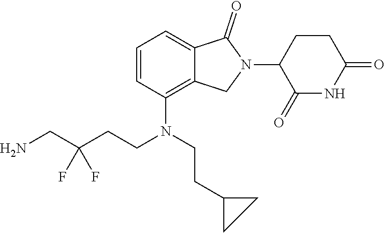 [SMILES: NCC(F)(F)CCN(CCC1CC1)c1cccc2c1CN(C1CCC(=O)NC1=O)C2=O]

To a solution of tert-butyl N-{4-[(2-cyclopropylethyl)[2-(2,6-dioxopiperidin-3-yl)-1-oxo-3H-isoindol-4-yl]amino]-2,2-difluorobutyl}carbamate 7 (110 mg, 0.206 mmol, 1 equiv) in DCM (1.65 mL, 25.985 mmol, 126.14 equiv) was added TFA (0.33 mL, 4.448 mmol, 21.59 equiv) at room temperature under nitrogen atmosphere. The resulting mixture was stirred for 2 h at room temperature under nitrogen atmosphere. The resulting mixture was concentrated under reduced pressure. The residue was purified by reverse flash chromatography with the following conditions: column, C18 silica gel; mobile phase, MeCN in Water (0.1% TFA), 0% to 100% gradient in 10 min; detector, UV 254 nm to afford 3-{4-[(4-amino-3,3-difluorobutyl)(2-cyclopropylethyl)amino]-1-oxo-3H-isoindol-2-yl}piperidine-2,6-dione (70 mg, 78.30%) as white solid.

¹H-NMR (400 MHz, DMSO-d₆): δ 7.49 (td, J=7.7, 2.3 Hz, 1H), 7.34 (dd, J=7.4, 3.3 Hz, 1H), 7.20 (dd, J=8.1, 3.0 Hz, 1H), 5.08 (ddd, J=13.4, 5.2, 2.9 Hz, 1H), 4.53-4.30 (m, 2H), 3.57-3.35 (m, 4H), 3.29 (t, J=7.2 Hz, 2H), 2.87 (dd, J=13.4, 4.9 Hz, 1H), 2.75-2.63 (m, 1H), 2.47 (tt, J=13.2, 6.6 Hz, 1H), 2.23 (dt, J=18.1, 9.9 Hz, 2H), 2.14-2.00 (m, 1H), 1.46-1.22 (m, 2H), 0.66 (s, 1H), 0.38 (dt, J=8.5, 2.8 Hz, 2H), 0.00 (dd, J=4.8, 2.0 Hz, 2H).

LCMS:435.2 (M+H). Method: Mobile Phase A: 0.05% TFA in H₂O. Mobile Phase B: Acetonitrile. Flow rate: 1.8 mL/min. COLUMN: Ascentis Express C18 (30×3.0) mm, 5μ. Rt (min): 0.974; Area %—99.31.

### Compound 519

### 3-(4-{[4-amino-3-(trifluoromethyl)butyl](2-cyclopropylethyl)amino}-1-oxo-3H-isoindol-2-yl)piperidine-2,6-dione

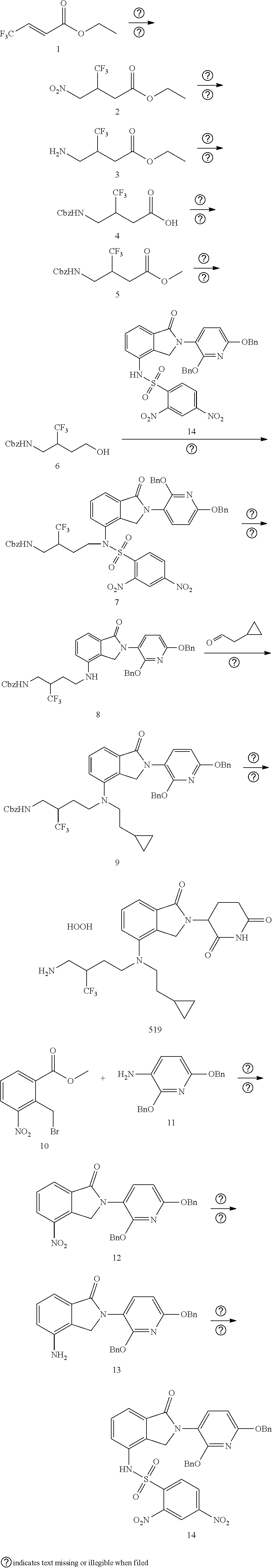

### Step-10: 2-[2,6-bis(benzyloxy)pyridin-3-yl]-4-nitro-3H-isoindol-1-one

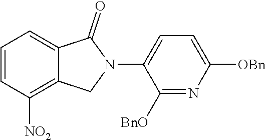 [SMILES: O=C1c2cccc([N+](=O)[O-])c2CN1c1ccc(OCc2ccccc2)nc1OCc1ccccc1]

A solution of methyl 2-(bromomethyl)-3-nitrobenzoate 10 (1 g, 3.649 mmol, 1 equiv), 2,6-bis(benzyloxy)pyridin-3-amine 11 (1.7 g, 5.549 mmol, 1.52 equiv) and DIEA (2.4 g, 18.569 mmol, 5.09 equiv) in DMF (15 mL, 193.823 mmol, 53.12 equiv) was stirred for overnight at room temperature under nitrogen atmosphere. The resulting mixture was extracted with EtOAc (3×200 ml). The combined organic layers were washed with water (2×200 mL), dried over Na₂SO₄, filtered and concentrated. The residue was purified by silica gel column chromatography, eluted with PE/EA (5:1) to afford 2-[2,6-bis(benzyloxy)pyridin-3-yl]-4-nitro-3H-isoindol-1-one 12 as a light yellow solid.

LCMS:468.2 (M+H). Method: Mobile Phase A: 0.1% Formic Acid in H₂O. Mobile Phase B: 0.1% Formic Acid in Acetonitrile. Flow rate: 1.5 mL/min. COLUMN: Shim-pack Scepter C18 (11×33) mm, 5 u. Rt (min):1.322; Area %—98.66.

### Step-11: 4-amino-2-[2,6-bis(benzyloxy)pyridin-3-yl]-3H-isoindol-1-one

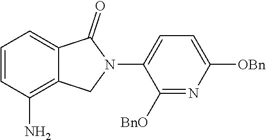 [SMILES: Nc1cccc2c1CN(c1ccc(OCc3ccccc3)nc1OCc1ccccc1)C2=O]

A solution of 2-[2,6-bis(benzyloxy)pyridin-3-yl]-4-nitro-3H-isoindol-1-one 12 (1.1 g, 2.353 mmol, 1 equiv), Zn (0.76 g, 11.624 mmol, 4.94 equiv) and NH₄Cl (1.25 g, 23.369 mmol, 9.93 equiv) in MeOH (11 mL, 271.687 mmol, 115.46 equiv) was stirred for overnight at 80° C. under nitrogen atmosphere. The resulting mixture was filtered, the filter cake was washed with MeOH (5×300 mL). The filtrate was concentrated under reduced pressure. The resulting mixture was concentrated under reduced pressure. The residue was purified by silica gel column chromatography, eluted with PE/EA (5:1) to afford 4-amino-2-[2,6-bis(benzyloxy)pyridin-3-yl]-3H-isoindol-1-one 13 as a yellow solid.

LCMS:438.1 (M+H). Method: Mobile Phase A: 0.1% Formic Acid in H₂O. Mobile Phase B: Acetonitrile. Flow rate: 1.5 mL/min. COLUMN: Express C18 (30×3) mm, 4 u. Rt (min):0.859; Area %—99.02.

### Step-12: N-{2-[2,6-bis(benzyloxy)pyridin-3-yl]-1-oxo-3H-isoindol-4-yl}-2,4-dinitrobenzenesulfonamide

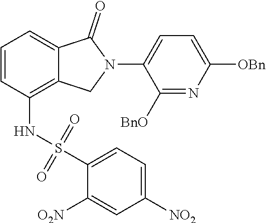 [SMILES: O=C1c2cccc(NS(=O)(=O)c3ccc([N+](=O)[O-])cc3[N+](=O)[O-])c2CN1c1ccc(OCc2ccccc2)nc1OCc1ccccc1]

A solution of 4-amino-2-[2,6-bis(benzyloxy)pyridin-3-yl]-3H-isoindol-1-one 13 (1 g, 2.286 mmol, 1 equiv) and Et₃N (1.2 g, 11.859 mmol, 5.19 equiv) in DCM (10 mL, 157.306 mmol, 68.82 equiv) was stirred for 30 min at 0° C. under nitrogen atmosphere. To the above mixture was added 2,4-dinitrobenzenesulfonyl chloride (1.8 g, 6.751 mmol, 2.95 equiv) dropwise over 10 min at 0° C. The resulting mixture was stirred for additional 2 h at room temperature. The resulting mixture was extracted with CH₂Cl₂ (3×200 mL). The combined organic layers were washed with water (2×200 mL), dried over Na₂SO₄, filtered and concentrated. The residue was purified by silica gel column chromatography, eluted with PE/EA (5:1) to afford N-{2-[2,6-bis(benzyloxy)pyridin-3-yl]-1-oxo-3H-isoindol-4-yl}-2,4-dinitrobenzenesulfonamide 14 as a yellow solid.

LCMS:667.9 (M+H). Method: Mobile Phase A: 0.1% Formic Acid in H₂O. Mobile Phase B: 0.1% Formic Acid in Acetonitrile. Flow rate: 1.5 mL/min. COLUMN: HPH (50×3) mm, 4 u. Rt (min):1.273; Area %—96.41.

### Step-1: ethyl 4,4,4-trifluoro-3-(nitromethyl)butanoate

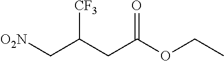

A solution of ethyl (2E)-4,4,4-trifluorobut-2-enoate 1 (5 g, 29.742 mmol, 1 equiv) and nitromethane (8 mL) was stirred for 10 min at 0° C. under nitrogen atmosphere. To the above mixture was added 1,1,3,3-tetramethylguanidine (0.75 mL, 6.512 mmol, 0.22 equiv) dropwise over 10 min at 0° C. The resulting mixture was stirred for additional 3 h at room temperature. The resulting mixture was extracted with EtOAc (3×300 mL). The combined organic layers were washed with brine (2×300 mL), dried over Na₂SO₄, filtered and concentrated. The crude product was purified by distillation under reduced pressure and the fraction was collected at 80° C. This resulted in ethyl 4,4,4-trifluoro-3-(nitromethyl)butanoate 2 (3.4 g, 48.42%) as colorless oil.

LCMS: No mass signal on LCMS.

### Step-2: ethyl 4-amino-3-(trifluoromethyl)butanoate

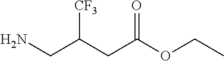

A solution of ethyl 4,4,4-trifluoro-3-(nitromethyl)butanoate 2 (3.4 g, 14.837 mmol, 1 equiv) and Pd/C (0.34 g, 3.195 mmol, 0.22 equiv) in THF (30 mL, 740.965 mmol, 49.94 equiv) was stirred for overnight at room temperature under hydrogen atmosphere. The resulting mixture was filtered, the filter cake was washed with THF (3×100 mL). The filtrate was concentrated under reduced pressure. The crude product was used in the next step directly without further purification.

LCMS:200.3 (M+H). Method: Mobile Phase A: 0.1% Formic Acid in H₂O. Mobile Phase B: 0.1% Formic Acid in Acetonitrile. Flow rate: 1.5 mL/min. COLUMN: HPH (50×3) mm, 4 u. Rt (min):0.672; Area %—95.37.

### Step-3: 3-(4-((4-Aminobutyl)amino)-1-oxoisoindolin-2-yl)piperidine-2,6-dione hydrochloride

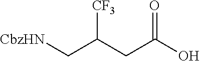 [SMILES: O=C(O)CC(CNC(=O)OCc1ccccc1)C(F)(F)F]

A solution of ethyl 4-amino-3-(trifluoromethyl)butanoate 3 (3.4 g, 17.071 mmol, 1 equiv) and NaOH (2.71 g, 67.772 mmol, 3.97 equiv) in THF (17.29 mL, 213.388 mmol, 12.50 equiv) and H₂O (17.29 mL, 959.732 mmol, 56.22 equiv) was stirred for 30 min at 0° C. under nitrogen atmosphere. To the above mixture was added CbzCl (3.17 g, 18.607 mmol, 1.09 equiv) in portions over 10 min at 0° C. The resulting mixture was stirred for additional 1 h at room temperature. The residue was purified by reverse flash chromatography with the following conditions: column, C18 silica gel; mobile phase, ACN in water (0.1% FA), 10% to 50% gradient in 20 min; detector, UV 254 nm. This resulted in 3-({[(benzyloxy)carbonyl]amino}methyl)-4,4,4-trifluorobutanoic acid 4 (1.5 g, 28.79%) as off-white solid.

LCMS:306.1 (M+H). Method: Mobile Phase A: 0.1% Formic Acid in H₂O. Mobile Phase B: 0.1% Formic Acid in Acetonitrile. Flow rate: 1.5 mL/min. COLUMN: Halo C18 (30×3) mm, 4 u. Rt (min):0.824; Area %—68.03.

### Step-4: methyl 3-({[(benzyloxy)carbonyl]amino}methyl)-4,4,4-trifluorobutanoate

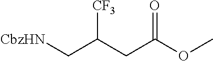 [SMILES: COC(=O)CC(CNC(=O)OCc1ccccc1)C(F)(F)F]

A solution of 3-({[(benzyloxy)carbonyl]amino}methyl)-4,4,4-trifluorobutanoic acid 4 (1.5 g, 4.914 mmol, 1 equiv) in MeOH (15 mL, 370.482 mmol, 75.39 equiv) was stirred for 10 min at 0° C. under nitrogen atmosphere. To the above mixture was added SOCl₂ (2.92 g, 24.570 mmol, 5.00 equiv) dropwise 10 min at 0° C. The resulting mixture was stirred for additional 4 h at 80° C. The resulting mixture was concentrated under reduced pressure. The residue was purified by reverse flash chromatography with the following conditions: column, C18 silica gel; mobile phase, MeCN in water (0.1% FA), 10% to 50% gradient in 10 min; detector, UV 254 nm. This resulted in methyl 3-({[(benzyloxy)carbonyl]amino}methyl)-4,4,4-trifluorobutanoate 5 (500 mg, 31.87%) as off-white solid.

LCMS:320.1 (M+H). Method: Mobile Phase A: 0.1% Formic Acid in H₂O. Mobile Phase B: 0.1% Formic Acid in Acetonitrile. Flow rate: 1.5 mL/min. COLUMN: HPH (50×3) mm, 4 u. Rt (min):1.029; Area %—100.0.

### Step-5: benzyl N-[4-hydroxy-2-(trifluoromethyl)butyl]carbamate

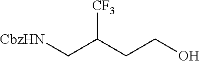 [SMILES: O=C(NCC(CCO)C(F)(F)F)OCc1ccccc1]

A solution of methyl 3-({[(benzyloxy)carbonyl]amino}methyl)-4,4,4-trifluorobutanoate 5 (500 mg, 1.566 mmol, 1 equiv) in CH₃OH (12.50 mL, 308.737 mmol, 197.15 equiv) was stirred for 10 min at 0° C. under nitrogen atmosphere. To the above mixture was added NaBH₄ (1.8 g, 47.581 mmol, 30.38 equiv) in portions over 5 min at 0° C. The resulting mixture was stirred for additional 2 h at room temperature. The reaction was quenched with water at 0° C. The resulting mixture was extracted with EtOAc (3×100 mL). The combined organic layers were washed with brine (2×100 mL), dried over Na₂SO₄, filtered and concentrated. The residue was purified by reverse flash chromatography with the following conditions: column, C18 silica gel; mobile phase, MeCN in water (0.1% FA), 10% to 50% gradient in 10 min; detector, UV 254 nm. This resulted in benzyl N-[4-hydroxy-2-(trifluoromethyl)butyl]carbamate 6 (400 mg, 87.69%) as light yellow oil.

LCMS:292.2 (M+H). Method: Mobile Phase A: 0.1% Formic Acid in H₂O. Mobile Phase B: 0.1% Formic Acid in Acetonitrile. Flow rate: 1.5 mL/min. COLUMN: Halo (2×30) mm, 5 u. Rt (min):0.098; Area %—93.79.

### Step-6: benzyl N-[4-(N-{2-[2,6-bis(benzyloxy)pyridin-3-yl]-1-oxo-3H-isoindol-4-yl}2,4-dinitrobenzenesulfonamido)-2-(trifluoromethyl)butyl]carbamate

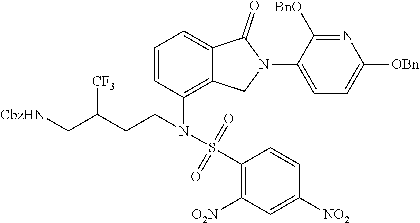 [SMILES: O=C(NCC(CCN(c1cccc2c1CN(c1ccc(OCc3ccccc3)nc1OCc1ccccc1)C2=O)S(=O)(=O)c1ccc([N+](=O)[O-])cc1[N+](=O)[O-])C(F)(F)F)OCc1ccccc1]

A solution of benzyl N-[4-hydroxy-2-(trifluoromethyl)butyl]carbamate 6 (300 mg, 1.030 mmol, 3 equiv), N-{2-[2,6-bis(benzyloxy)pyridin-3-yl]-1-oxo-3H-isoindol-4-yl}-2,4-dinitrobenzenesulfonamide 14 (175.74 mg, 0.263 mmol, 0.33 equiv) and PPh₃ (180.10 mg, 0.687 mmol, 2 equiv) in THF (2 mL, 24.686 mmol, 23.97 equiv) was stirred for 10 min at 0° C. under nitrogen atmosphere. To the above mixture was added DIAD (138.85 mg, 0.687 mmol, 2 equiv) dropwise over 10 min at 0° C. The resulting mixture was stirred for additional overnight at room temperature. The resulting mixture was extracted with EtOAc (3×100 mL). The combined organic layers were washed with brine (2×100 mL), dried over Na₂SO₄, filtered and concentrated. The residue was purified by reverse flash chromatography with the following conditions: column, C18 silica gel; mobile phase, MeCN in water (0.1% FA), 10% to 50% gradient in 10 min; detector, UV 254 nm. This resulted in benzyl N-[4-(N-{2-[2,6-bis(benzyloxy)pyridin-3-yl]-1-oxo-3H-isoindol-4-yl}2,4-dinitrobenzenesulfonamido)-2-(trifluoromethyl)butyl]carbamate 7 (200 mg, 61.91%) as off-white solid.

LCMS:941.2 (M+H). Method: Mobile Phase A: 0.1% Formic Acid in H₂O. Mobile Phase B: 0.1% Formic Acid in Acetonitrile. Flow rate: 1.5 mL/min. COLUMN: Halo C18 (30×3) mm, 4 u. Rt (min):1.309; Area %—98.91.

### Step-7: benzyl N-[4-({2-[2,6-bis(benzyloxy)pyridin-3-yl]-1-oxo-3H-isoindol-4-yl}amino)-2-(trifluoromethyl)butyl]carbamate

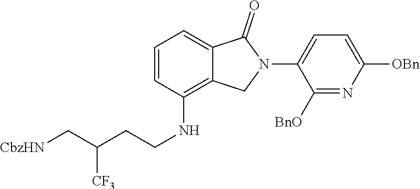 [SMILES: O=C(NCC(CCNc1cccc2c1CN(c1ccc(OCc3ccccc3)nc1OCc1ccccc1)C2=O)C(F)(F)F)OCc1ccccc1]

A solution of benzyl N-[4-(N-{2-[2,6-bis(benzyloxy)pyridin-3-yl]-1-oxo-3H-isoindol-4-yl}2,4-dinitrobenzenesulfonamido)-2-(trifluoromethyl)butyl]carbamate 7 (200 mg, 0.213 mmol, 1 equiv), HSCCOOH (58.67 mg, 0.639 mmol, 3 equiv) and Et₃N (107.55 mg, 1.065 mmol, 5 equiv) in DMF (3.6 mL) was stirred for 2 h at room temperature under nitrogen atmosphere. The residue was purified by reverse flash chromatography with the following conditions: column, C18 silica gel; mobile phase, MeCN in water (0.1% FA), 10% to 50% gradient in 10 min; detector, UV 254 nm. This resulted in benzyl N-[4-({2-[2,6-bis(benzyloxy)pyridin-3-yl]-1-oxo-3H-isoindol-4-yl}amino)-2-(trifluoromethyl)butyl]carbamate 8 (120 mg, 79.43%) as light yellow solid.

LCMS:711.2 (M+H). Method: Mobile Phase A:0.4% NH₃H₂O in H₂O. Mobile Phase B: Acetonitrile. Flow rate: 1.5 mL/min. COLUMN: HPH C18 (50×3) mm, 4 u. Rt (min):1.605; Area %—97.97.

### Step-8: benzyl N-[4-({2-[2,6-bis(benzyloxy)pyridin-3-yl]-1-oxo-3H-isoindol-4-yl}(2-cyclopropylethyl)amino)-2-(trifluoromethyl)butyl]carbamate

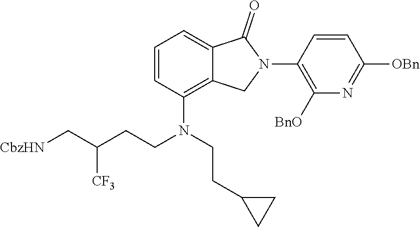 [SMILES: O=C(NCC(CCN(CCC1CC1)c1cccc2c1CN(c1ccc(OCc3ccccc3)nc1OCc1ccccc1)C2=O)C(F)(F)F)OCc1ccccc1]

A solution of benzyl N-[4-({2-[2,6-bis(benzyloxy)pyridin-3-yl]-1-oxo-3H-isoindol-4-yl}amino)-2-(trifluoromethyl)butyl]carbamate 8 (120 mg, 0.169 mmol, 1 equiv) and 2-cyclopropylacetaldehyde (70.87 mg, 0.843 mmol, 4.99 equiv) in DCM (3.00 mL, 47.237 mmol, 279.51 equiv) and DMF (0.72 mL, 9.312 mmol, 55.10 equiv) was stirred for 30 min at room temperature under nitrogen atmosphere. To the above mixture was added TFA (57.75 mg, 0.507 mmol, 3.00 equiv) dropwise over 10 min at room temperature. The resulting mixture was stirred for additional 3 h at room temperature. To the above mixture was added STAB (178.91 mg, 0.845 mmol, 5.00 equiv) in portions over 5 min at 0° C. The resulting mixture was stirred for additional overnight at room temperature. The resulting mixture was concentrated under reduced pressure. The residue was purified by reverse flash chromatography with the following conditions: column, C18 silica gel; mobile phase, MeCN in water (0.1% NH₃·H₂O), 10% to 50% gradient in 10 min; detector, UV 254 nm. This resulted in benzyl N-[4-({2-[2,6-bis(benzyloxy)pyridin-3-yl]-1-oxo-3H-isoindol-4-yl}(2-cyclopropylethyl)amino)-2-(trifluoromethyl)butyl]carbamate 9 (120 mg, 91.25%) as light yellow oil.

LCMS:779.3 (M+H). 0.1% Formic Acid in H₂O. Mobile Phase B: 0.1% Formic Acid in Acetonitrile. Flow rate: 1.5 mL/min. COLUMN: Halo(2×30) mm, 5 u. Rt (min):1.384; Area %—98.16.

### Step-9: 3-(4-{[4-amino-3-(trifluoromethyl)butyl](2-cyclopropylethyl)amino}-1-oxo-3H-isoindol-2-yl)piperidine-2,6-dione

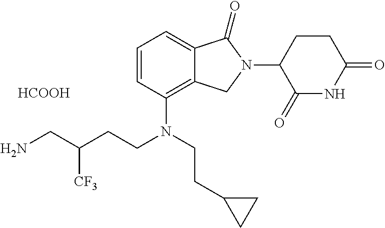 [SMILES: "HCOOH" ; NCC(CCN(CCC1CC1)c1cccc2c1CN(C1CCC(=O)NC1=O)C2=O)C(F)(F)F]

A solution of benzyl N-[4-({2-[2,6-bis(benzyloxy)pyridin-3-yl]-1-oxo-3H-isoindol-4-yl}(2-cyclopropylethyl)amino)-2-(trifluoromethyl)butyl]carbamate 9 (120 mg, 0.154 mmol, 1 equiv) and Pd/C (90.01 mg, 0.845 mmol, 5.49 equiv) in DMF (2.40 mL, 30.997 mmol, 201.28 equiv) was stirred for overnight at room temperature under hydrogen atmosphere. The resulting mixture was filtered, the filter cake was washed with DMF (2×3 mL). The filtrate was concentrated under reduced pressure. The residue was purified by reverse flash chromatography with the following conditions: column, C18 silica gel; mobile phase, MeCN in water (0.1% FA), 10% to 50% gradient in 10 min; detector, UV 254 nm. This resulted in 3-(4-{[4-amino-3-(trifluoromethyl)butyl](2-cyclopropylethyl)amino}-1-oxo-3H-isoindol-2-yl)piperidine-2,6-dione (60 mg, 83.48%) as white solid.

LCMS:467.3 (M+H). Method: Mobile Phase A: 0.1% NH₄HCO₃ in H₂O. Mobile Phase B: MEOH. Flow rate: 1.5 mL/min. COLUMN: Poroshell HPH-C18 (50×3) mm, 4 u. Rt (min):0.624; Area %—96.01.

¹H-NMR (400 MHz, DMSO-d₆): δ 8.32 (s, 1H), 7.42 (t, J=7.7 Hz, 1H), 7.25 (d, J=7.3 Hz, 1H), 7.14 (d, J=8.1 Hz, 1H), 5.10 (ddd, J=13.4, 5.2, 2.4 Hz, 1H), 4.52-4.25 (m, 2H), 3.30 (dt, J=14.5, 6.6 Hz, 4H), 2.99-2.74 (m, 3H), 2.63 (ddd, J=17.4, 4.5, 2.3 Hz, 1H), 2.45 (ddd, J=17.7, 12.8, 6.2 Hz, 2H), 2.03 (dp, J=11.8, 3.8 Hz, 1H), 1.74 (h, J=6.8 Hz, 2H), 1.32 (tp, J=13.6, 7.0 Hz, 2H), 0.65 (pd, J=6.8, 3.3 Hz, 1H), 0.44-0.31 (m, 2H), 0.01 (m, 2H).

### Compound 521

### 3-(4-(((1R,3R,4R)-4-amino-3-fluorocyclohexyl)(2-cyclopropylethyl)amino)-1-oxoisoindolin-2-yl)piperidine-2,6-dione; formic acid

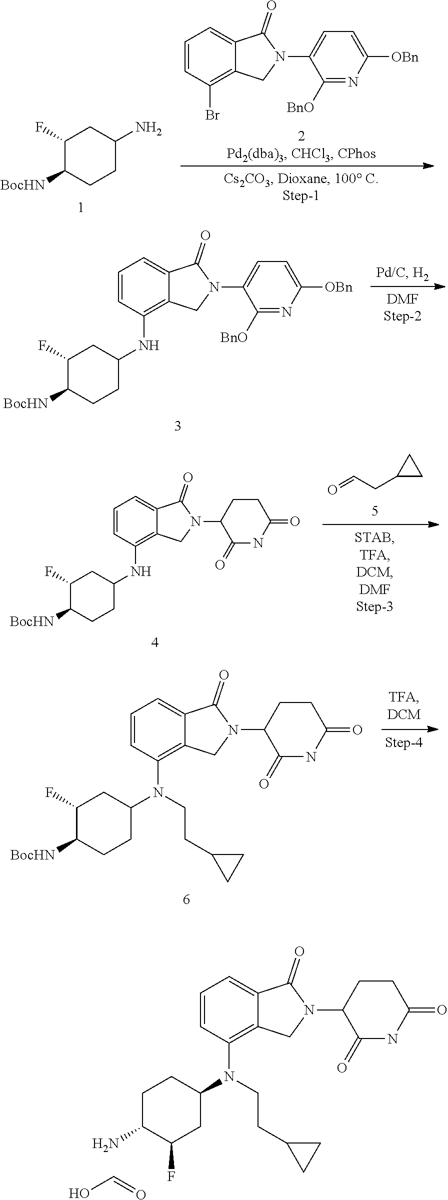

### Step-1:tert-butyl((1R,2R,4R)-4-((2-(2,6-bis(benzyloxy)pyridin-3-yl)-1-oxoisoindolin-4-yl)amino)-2-fluorocyclohexyl)carbamate

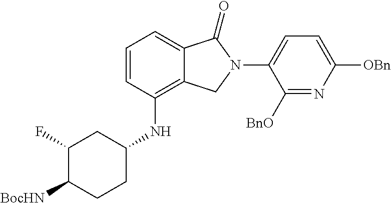 [SMILES: CC(C)(C)OC(=O)N[C@@H]1CC[C@@H](Nc2cccc3c2CN(c2ccc(OCc4ccccc4)nc2OCc2ccccc2)C3=O)C[C@H]1F]

To a stirred mixture of tert-butyl N-[(1R,2R,4R)-4-amino-2-fluorocyclohexyl]carbamate hydrochloride 1 (500 mg, 1.860 mmol, 1 equiv) and 2-[2,6-bis(benzyloxy)pyridin-3-yl]-4-bromo-3H-isoindol-1-one 2 (1119.32 mg, 2.232 mmol, 1.2 equiv) in dioxane (10 mL) was add Pd₂(dba)₃·CHCl₃ (192.57 mg, 0.186 mmol, 0.1 equiv), CPhos (162.46 mg, 0.372 mmol, 0.2 equiv) and Cs₂CO₃ (2424.61 mg, 7.440 mmol, 4 equiv). The resulting mixture was stirred overnight at 100° C. under nitrogen atmosphere. The mixture was allowed to cool down to room temperature, filtered, and the filter cake was washed with dioxane (3×10 mL). The filtrate was concentrated under reduced pressure, and purified by silica gel column chromatography, eluted with PE/EA (1:1) to afford tert-butyl((1R,2R,4R)-4-((2-(2,6-bis(benzyloxy)pyridin-3-yl)-1-oxoisoindolin-4-yl)amino)-2-fluorocyclohexyl)carbamate 3 (990 mg, 81.52%) as brown solid.

LCMS: 652.1 (M+H). Method: Mobile Phase A: Mobile Phase A: Water/5 mM NH₄HCO₃. Mobile Phase B: Acetonitrile. Flow rate: 1.5 mL/min. COLUMN: HPHC18 (50×3.0) mm, 2.7μ. Rt (min): 0.895; Area %—96.97.

### Step-2:tert-butyl ((1R,2R,4R)-4-((2-(2,6-dioxopiperidin-3-yl)-1-oxoisoindolin-4-yl)amino)-2-fluorocyclohexyl)carbamate

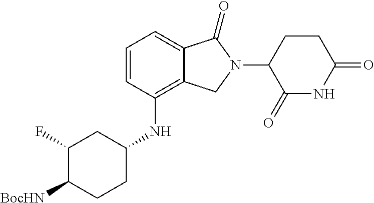 [SMILES: CC(C)(C)OC(=O)N[C@@H]1CC[C@@H](Nc2cccc3c2CN(C2CCC(=O)NC2=O)C3=O)C[C@H]1F]

To a solution of tert-butyl((1R,2R,4R)-4-((2-(2,6-bis(benzyloxy)pyridin-3-yl)-1-oxoisoindolin-4-yl)amino)-2-fluorocyclohexyl)carbamate 3 (500 mg, 0.766 mmol, 1 equiv) in 5 mL DMF was added Pd/C (200 mg) in a pressure tank. The mixture was hydrogenated at room temperature under 5 psi of hydrogen pressure for overnight, filtered through a Celite pad and concentrated under reduced pressure. The residue was purified by reverse flash chromatography with the following conditions: column, C18 silica gel; mobile phase, MeCN in Water (0.1% FA), 0% to 100% gradient in 20 min; detector, UV 254 nm. This resulted in tert-butyl ((1R,2R,4R)-4-((2-(2,6-dioxopiperidin-3-yl)-1-oxoisoindolin-4-yl)amino)-2-fluorocyclohexyl)carbamate 4 (360 mg, 99.04%) as off-white solid.

LCMS: 475.1 (M+H). Method: Mobile Phase A: Water/0.1% FA. Mobile Phase B: Acetonitrile/0.1% FA. Flow rate: 1.5 mL/min. COLUMN: XB-C18 (30×3.0) mm, 1.7μ. Rt (min): 0.826; Area %—100.0.

### Step-3: tert-butyl ((1R,2R,4R)-4-((2-cyclopropylethyl)(2-(2,6-dioxopiperidin-3-yl)-1-oxoisoindolin-4-yl)amino)-2-fluorocyclohexyl)carbamate

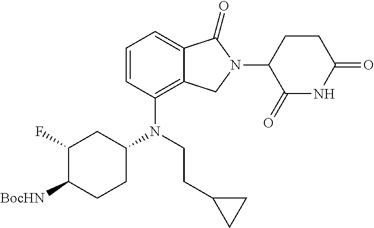 [SMILES: CC(C)(C)OC(=O)N[C@@H]1CC[C@@H](N(CCC2CC2)c2cccc3c2CN(C2CCC(=O)NC2=O)C3=O)C[C@H]1F]

A solution of tert-butyl ((1R,2R,4R)-4-((2-(2,6-dioxopiperidin-3-yl)-1-oxoisoindolin-4-yl)amino)-2-fluorocyclohexyl)carbamate 4 (100 mg, 0.211 mmol, 1 equiv), 2-cyclopropylacetaldehyde 5 (88.63 mg, 1.055 mmol, 5 equiv) and TFA (72.09 mg, 0.633 mmol, 3 equiv) in DCM (2.5 mL) and DMF (0.6 mL) was stirred for 2 h at room temperature under nitrogen atmosphere followed by the addition of STAB (223.31 mg, 1.055 mmol, 5 equiv) dropwise at 0° C. The resulting mixture was stirred overnight at room temperature under nitrogen atmosphere. The reaction was quenched with water at room temperature. The resulting mixture was extracted with CH₂Cl₂ (3×20 mL). The combined organic layers were washed with brine (2×20 mL), dried over anhydrous Na₂SO₄, filtered and concentrated under reduced pressure. The residue was purified by silica gel column chromatography, eluted with PE/EA (2:1) to afford tert-butyl ((1R,2R,4R)-4-((2-cyclopropylethyl)(2-(2,6-dioxopiperidin-3-yl)-1-oxoisoindolin-4-yl)amino)-2-fluorocyclohexyl)carbamate 6 (100 mg, 87.45%) as yellow solid.

LCMS: 543.1 (M+H). Method: Mobile Phase A: Water/0.1% FA. Mobile Phase B: Acetonitrile/0.1% FA. Flow rate: 1.5 mL/min. COLUMN: XB-C18 (30×3.0) mm, 1.7μ. Rt (min): 1.016; Area %—100.0.

### Step-4: 3-(4-(((1R,3R,4R)-4-amino-3-fluorocyclohexyl)(2-cyclopropylethyl)amino)-1-oxoisoindolin-2-yl)piperidine-2,6-dione; formic acid

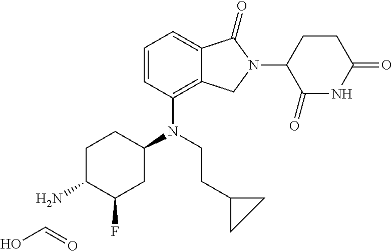

To a stirred solution of tert-butyl ((1R,2R,4R)-4-((2-cyclopropylethyl)(2-(2,6-dioxopiperidin-3-yl)-1-oxoisoindolin-4-yl)amino)-2-fluorocyclohexyl)carbamate 6 (100 mg, 0.184 mmol, 1 equiv) in DCM (3 mL) was added TFA (0.6 ml) dropwise at 0° C. under nitrogen atmosphere. The resulting mixture was stirred for 3 h at room temperature under nitrogen atmosphere, concentrated under vacuum, and purified by reverse flash chromatography with the following conditions: column, C18 silica gel; mobile phase, MeCN in Water (0.1% FA), 0% to 100% gradient in 10 min; detector, UV 254 nm. This resulted in 3-(4-(((1R,3R,4R)-4-amino-3-fluorocyclohexyl)(2-cyclopropylethyl)amino)-1-oxoisoindolin-2-yl)piperidine-2,6-dione; formic acid (54.7 mg, 67.08%) as a yellow solid.

¹H-NMR (400 MHz, DMSO-d₆): δ 11.01 (s, 1H), 8.39 (s, 1H), 7.49 (t, J=7.6 Hz, 1H), 7.37 (dd, J=12.4, 7.7 Hz, 2H), 5.17 (ddd, J=12.8, 5.1, 1.9 Hz, 5H), 4.62-4.17 (m, 3H), 3.44-3.16 (m, 3H), 3.12-2.85 (m, 2H), 2.76-2.59 (m, 2H), 2.30-1.59 (m, 6H), 1.41-1.08 (m, 3H), 0.70 (pd, J=7.1, 3.5 Hz, 1H), 0.40 (dt, J=8.1, 2.8 Hz, 2H). LCMS: 443.20 (M+H). Method: Mobile Phase A: Water/5 mM NH₄HCO₃. Mobile Phase B: Acetonitrile. Flow rate: 1.5 mL/min. COLUMN: Kinetex EVO C18 (50×3.0) mm, 2.7μ. Rt (min): 1.353; Area %—96.91.

### Compound 522

### 3-(4-{[(3R,6R)-6-(aminomethyl)oxan-3-yl](2-cyclopropylethyl)amino}-1-oxo-3H-isoindol-2-yl)piperidine-2,6-dione

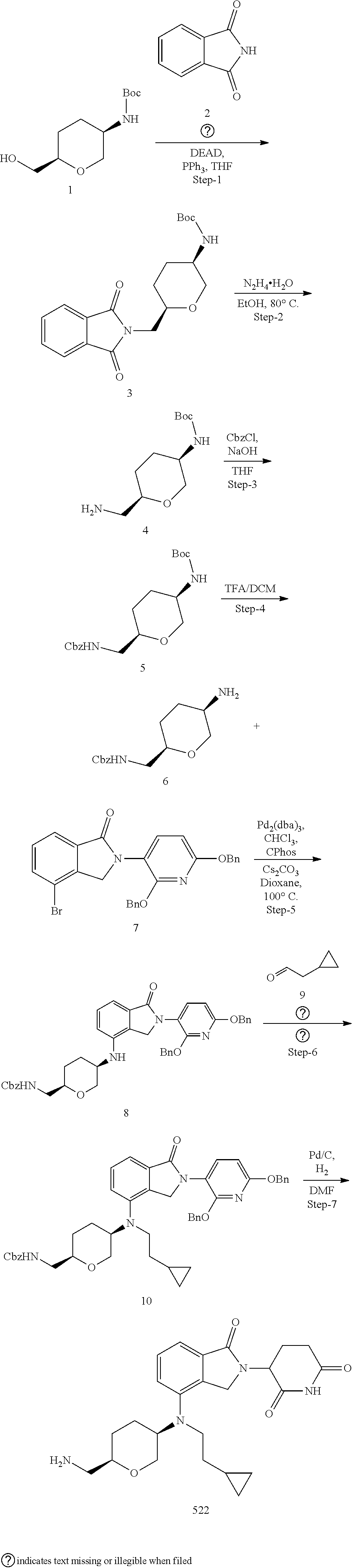

### Step-1: tert-butyl N-[(3R,6R)-6-[(1,3-dioxoisoindol-2-yl)methyl]oxan-3-yl]carbamate

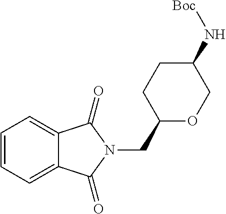 [SMILES: CC(C)(C)OC(=O)N[C@@H]1CC[C@H](CN2C(=O)c3ccccc3C2=O)OC1]

Into a 250 mL round-bottom flask were added tert-butyl N-[(3R,6R)-6-(hydroxymethyl)oxan-3-yl]carbamate 1 (950 mg, 4.107 mmol, 1 equiv), phthalimide 2 (906.49 mg, 6.161 mmol, 1.5 equiv), PPh₃ (1615.99 mg, 6.161 mmol, 1.5 equiv) and THF (10 mL) at room temperature. The resulting mixture was stirred for 0.2 h at room temperature. DEAD (1072.98 mg, 6.161 mmol, 1.5 equiv) was added at 0° C. The resulting mixture was stirred for 5 h at room temperature. Desired product could be detected by LCMS. The resulting mixture was extracted with EA (3×100 ml). The combined organic layers were washed with H₂O (3×100 ml), dried over anhydrous Na₂SO₄. filtered and concentrated. The residue was purified by reverse flash chromatography with the following conditions: column, C18 silica gel; mobile phase, ACN in Water (0.1% FA); detector, UV 254 nm to afford tert-butyl N-[(3R,6R)-6-[(1,3-dioxoisoindol-2-yl)methyl]oxan-3-yl]carbamate 3 (400 mg, 26.48%) as yellow oil.

LCMS: 261.2 (M+H). Method: Mobile Phase A: 0.1% FA in H₂O. Mobile Phase B: Acetonitrile. Flow rate: 1.5 mL/min. COLUMN: Atlantis dC₁₈ (250×4.6) mm, 5 μm. Rt (min): 0.912; Area % —16.04.

### Step-2: tert-butyl N-[(3R,6R)-6-(aminomethyl)oxan-3-yl]carbamate

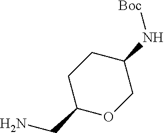 [SMILES: CC(C)(C)OC(=O)N[C@@H]1CC[C@H](CN)OC1]

Into a 250 mL round-bottom flask were added tert-butyl N-[(3R,6R)-6-[(1,3-dioxoisoindol-2-yl)methyl]oxan-3-yl]carbamate 3 (350 mg, 0.971 mmol, 1 equiv), diimine hydrate hydrogen (238.18 mg, 4.855 mmol, 5 equiv) and EtOH (10 mL) at room temperature. The resulting mixture was stirred for 6 h at 80° C. Desired product could be detected by LCMS. The resulting mixture was concentrated under vacuum. The residue was purified by reverse flash chromatography with the following conditions: column, C18 120 silica gel; mobile phase, ACN in Water (0.1% FA); detector, UV 254 nm to afford tert-butyl N-[(3R,6R)-6-(aminomethyl)oxan-3-yl]carbamate 4 (200 mg, 89.42%) as white solid.

LCMS: 231.2 (M+H). Method: Mobile Phase A: 0.1% FA in H₂O. Mobile Phase B: Acetonitrile. Flow rate: 1.5 mL/min. COLUMN: Atlantis dC₁₈ (250×4.6) mm, 5 μm. Rt (min): 0.848; Area % —94.22.

### Step-3: benzyl N-{[(2R,5R)-5-[(tert-butoxycarbonyl)amino]oxan-2-yl]methyl}carbamate

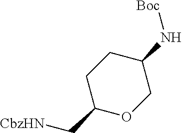 [SMILES: CC(C)(C)OC(=O)N[C@@H]1CC[C@H](CNC(=O)OCc2ccccc2)OC1]

Into a 250 mL round-bottom flask were added tert-butyl N-[(3R,6R)-6-(aminomethyl)oxan-3-yl]carbamate 4 (290 mg, 1.259 mmol, 1 equiv), benzyl chloroformate (322.21 mg, 1.888 mmol, 1.5 equiv), NaOH (100.73 mg, 2.518 mmol, 2 equiv) and THF (10 mL) at room temperature. The resulting mixture was stirred for 6 h at room temperature. Desired product could be detected by LCMS. The residue was purified by reverse flash chromatography with the following conditions: column, C18 120 silica gel; mobile phase, ACN in Water (0.1% FA); detector, UV 254 nm to afford benzyl N-{[(2R,5R)-5-[(tert-butoxycarbonyl)amino]oxan-2-yl]methyl}carbamate 5 (200 mg, 43.58%) as colorless oil.

LCMS: 265.2 (M+H). Method: Mobile Phase A: 0.1% FA in H₂O. Mobile Phase B: Acetonitrile. Flow rate: 1.5 mL/min. COLUMN: Atlantis dC₁₈ (250×4.6) mm, 5 μm. Rt (min): 0.928; Area %—7.55.

### Step-4: benzyl N-{[(2R,5R)-5-aminooxan-2-yl]methyl}carbamate

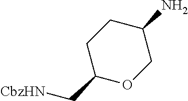 [SMILES: N[C@@H]1CC[C@H](CNC(=O)OCc2ccccc2)OC1]

Into a 250 mL round-bottom flask were added benzyl N-{[(2R,5R)-5-[(tert-butoxycarbonyl)amino]oxan-2-yl]methyl}carbamate 5 (200 mg, 0.549 mmol, 1 equiv) and DCM (10 mL) at room temperature. TFA (1 mL, 13.463 mmol, 24.53 equiv) was added at 0° C. The resulting mixture was stirred for 3 h at room temperature. Desired product could be detected by LCMS. The resulting mixture was concentrated under vacuum. This resulted in benzyl N-{[(2R,5R)-5-aminooxan-2-yl]methyl}carbamate 6 (130 mg, 89.62%) as yellow solid.

LCMS: 265.2 (M+H). Method: Mobile Phase A: 0.1% FA in H₂O. Mobile Phase B: Acetonitrile. Flow rate: 1.5 mL/min. COLUMN: Atlantis dC₁₈ (250×4.6) mm, 5 μm. Rt (min): 0.497; Area % —100.

### Step-5: benzyl N-{[(2R,5R)-5-({2-[2,6-bis(benzyloxy)pyridin-3-yl]-1-oxo-3H-isoindol-4-yl}amino)oxan-2-yl]methyl}carbamate

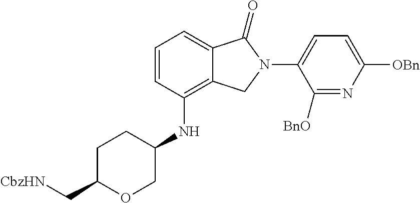 [SMILES: O=C(NC[C@H]1CC[C@@H](Nc2cccc3c2CN(c2ccc(OCc4ccccc4)nc2OCc2ccccc2)C3=O)CO1)OCc1ccccc1]

Into a 8 mL sealed tube were added benzyl N-{[(2R,5R)-5-aminooxan-2-yl]methyl}carbamate 6 (150 mg, 0.567 mmol, 1 equiv), 2-[2,6-bis(benzyloxy)pyridin-3-yl]-4-bromo-3H-isoindol-1-one 7 (341.43 mg, 0.680 mmol, 1.2 equiv), Cs₂CO₃ (554.69 mg, 1.701 mmol, 3 equiv), CPhos (49.56 mg, 0.113 mmol, 0.2 equiv), Pd₂(dba)₃·CHCl₃ (117.48 mg, 0.113 mmol, 0.2 equiv) and dioxane (1 mL) at 100° C. under nitrogen atmosphere. The resulting mixture was stirred for 10 h at 100° C. Desired product could be detected by LCMS. The resulting mixture was concentrated under vacuum. The residue was purified by reverse flash chromatography with the following conditions: column, C18 120 silica gel; mobile phase, ACN in Water (0.1% FA); detector, UV 254 nm to afford benzyl N-{[(2R,5R)-5-({2-[2,6-bis(benzyloxy)pyridin-3-yl]-1-oxo-3H-isoindol-4-yl}amino)oxan-2-yl]methyl}carbamate 8 (300 mg, 77.20%) as white solid.

LCMS: 685.3 (M+H). Method: Mobile Phase A: 0.1% FA in H₂O. Mobile Phase B: Acetonitrile. Flow rate: 1.5 mL/min. COLUMN: Atlantis dC₁₈ (250×4.6) mm, 5 μm. Rt (min): 1.203; Area % —51.48.

### Step-6: benzyl N-{[(2R,5R)-5-({2-[2,6-bis(benzyloxy)pyridin-3-yl]-1-oxo-3H-isoindol-4-yl}(2-cyclopropylethyl)amino)oxan-2-yl]methyl}carbamate

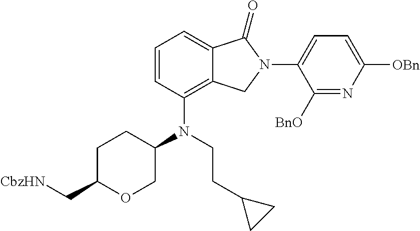 [SMILES: O=C(NC[C@H]1CC[C@@H](N(CCC2CC2)c2cccc3c2CN(c2ccc(OCc4ccccc4)nc2OCc2ccccc2)C3=O)CO1)OCc1ccccc1]

Into a 100 mL round-bottom flask were added benzyl N-{[(2R,5R)-5-({2-[2,6-bis(benzyloxy)pyridin-3-yl]-1-oxo-3H-isoindol-4-yl}amino)oxan-2-yl]methyl}carbamate 8 (150 mg, 0.219 mmol, 1 equiv), 2-cyclopropylacetaldehyde 9 (92.13 mg, 1.095 mmol, 5 equiv), DCM (1 mL) and DMF (0.24 mL) at room temperature. TFA (74.93 mg, 0.657 mmol, 3 equiv) was added at 0° C. The resulting mixture was stirred for 2 h at room temperature. STAB (232.12 mg, 1.095 mmol, 5 equiv) was added at 0° C. The resulting mixture was stirred for 8 h at room temperature. Desired product could be detected by LCMS. The resulting mixture was concentrated under vacuum. The resulting mixture was filtered, the filter cake was washed with DCM (3×20 ml). The filtrate was concentrated under reduced pressure. The residue was purified by reverse flash chromatography with the following conditions: column, C18 120 silica gel; mobile phase, ACN in Water (0.1% FA); detector, UV 254 nm to afford benzyl N-{[(2R,5R)-5-({2-[2,6-bis(benzyloxy)pyridin-3-yl]-1-oxo-3H-isoindol-4-yl}(2-cyclopropylethyl)amino)oxan-2-yl]methyl}carbamate 10 (100 mg, 60.64%) as yellow solid.

LCMS: 753.4 (M+H). Method: Mobile Phase A: 0.1% FA in H₂O. Mobile Phase B: Acetonitrile. Flow rate: 1.5 mL/min. COLUMN: Atlantis dC₁₈ (250×4.6) mm, 5 μm. Rt (min): 1.346; Area %—82.85.

### Step-7: 3-(4-{[(3R,6R)-6-(aminomethyl)oxan-3-yl](2-cyclopropylethyl)amino}-1-oxo-3H-isoindol-2-yl)piperidine-2,6-dione

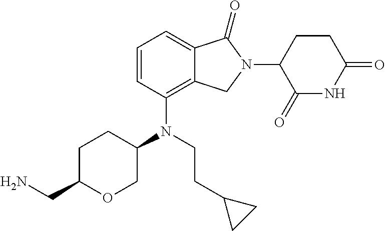 [SMILES: NC[C@H]1CC[C@@H](N(CCC2CC2)c2cccc3c2CN(C2CCC(=O)NC2=O)C3=O)CO1]

Into a 8 mL sealed tube were added benzyl N-{[(2R,5R)-5-({2-[2,6-bis(benzyloxy)pyridin-3-yl]-1-oxo-3H-isoindol-4-yl}(2-cyclopropylethyl)amino)oxan-2-yl]methyl}carbamate 10 (100 mg, 0.133 mmol, 1 equiv), Pd/C (141.34 mg, 1.330 mmol, 10 equiv) and dimethylformamide (1 mL) at room temperature. The resulting mixture was stirred for 3 h at room temperature. Desired product could be detected by LCMS. The resulting mixture was filtered, the filter cake was washed with DCM (3×20 ml). The filtrate was concentrated under reduced pressure. The residue was purified by reverse flash chromatography with the following conditions: column, C18 silica gel; mobile phase, ACN in Water (0.1% FA); detector, UV 254 nm. This resulted in 3-(4-{[(3R,6R)-6-(aminomethyl)oxan-3-yl](2-cyclopropylethyl)amino}-1-oxo-3H-isoindol-2-yl)piperidine-2,6-dione (20 mg, 32.57%) as white solid.

¹H-NMR (300 MHz, DMSO-d₆):δ 8.42 (s, 1H), 7.51-7.13 (m, 4H), 5.17-5.08 (m, 2H), 4.40 (d, J=14.4 Hz, 3H), 4.28 (d, J=17.6 Hz, 2H), 3.76 (t, J=11.6 Hz, 2H), 3.52 (t, J=14.4 Hz, 3H), 3.26 (d, J=16.7 Hz, 3H), 2.90 (s, 1H), 2.83 (s, 2H), 2.60 (d, J=17.9 Hz, 2H), 2.46 (s, 1H), 2.02 (s, 1H), 1.92 (s, 1H), 1.64 (s, 1H), 1.46 (s, 1H), 1.24 (s, 2H), 1.18-1.07 (m, 2H), 0.60 (s, 1H), 0.32 (d, J=7.9 Hz, 2H), −0.05-−0.12 (m, 2H). LCMS: 441.3 (M+H). Method: Mobile Phase A: 0.10% FA in H₂O. Mobile Phase B: Acetonitrile. Flow rate: 1.5 mL/min. COLUMN: Atlantis dC₁₈ (250×4.6) mm, 5 μm. Rt (min): 0.975; Area %—60.81.

### Compound 523

### 3-(4-(((1r,4r)-4-(aminomethyl)-4-hydroxycyclohexyl)(2-cyclopropylethyl)amino)-1-oxoisoindolin-2-yl)piperidine-2,6-dione

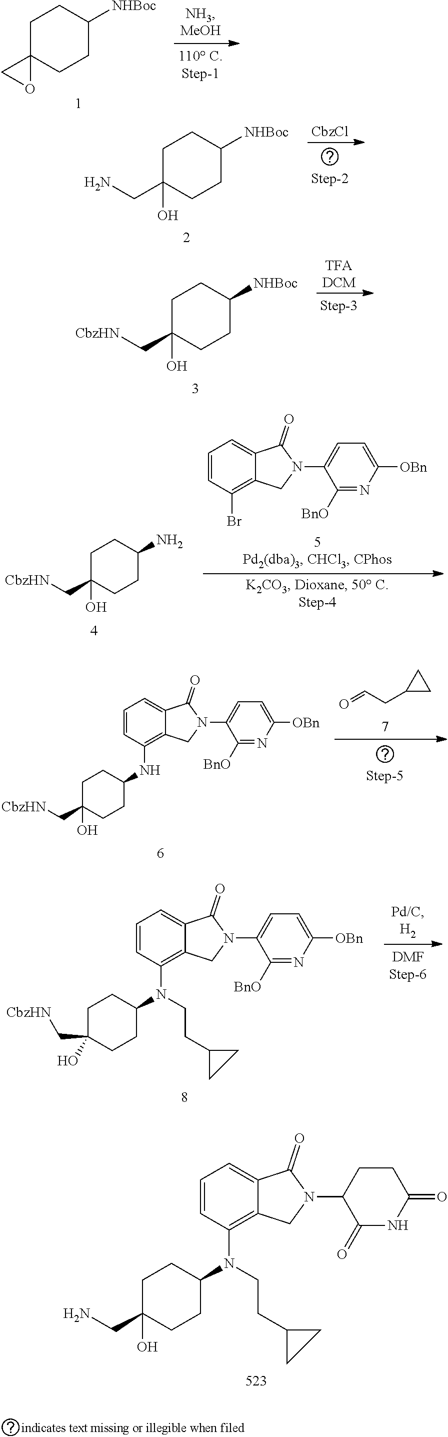

### Step-1:tert-butyl (4-(aminomethyl)-4-hydroxycyclohexyl)carbamate

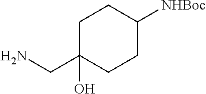 [SMILES: CC(C)(C)OC(=O)NC1CCC(O)(CN)CC1]

NH₃(g) in MeOH (25.00 mL, 880.760 mmol, 40.04 equiv) was added to a solution of tert-butyl (1-oxaspiro[2.5]octan-6-yl)carbamate 1 (5 g, 21.997 mmol, 1 equiv) in MeOH (15 mL) at room temperature. Then the mixture was sealed, heated to 110° C. and stirred for 2 hours. The mixture was allowed to cool down to room temperature. The solvent was removed in vacuum and the residue was triturated with CH₂Cl₂ (20 mL). The suspension was stirred for 30 min and filtered to give desired product (part I). The filtrate was concentrated, triturated with ethyl ether (20 mL) and filtered. The filter cake was washed with ethyl ether (2×10 mL) to afford desired product (part II). Two parts were combined to afford tert-butyl (4-(aminomethyl)-4-hydroxycyclohexyl)carbamate 2 (3.2 g, 59.54%) as a white solid.

LCMS: no mass signal.

### Step-2:tert-butyl((1r,4r)-4-((((benzyloxy)carbonyl)amino)methyl)-4-hydroxycyclohexyl)carbamate

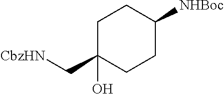 [SMILES: CC(C)(C)OC(=O)N[C@H]1CC[C@@](O)(CNC(=O)OCc2ccccc2)CC1]

A solution of tert-butyl N-[4-(aminomethyl)-4-hydroxycyclohexyl]carbamate (3.1 g, 12.687 mmol, 1 equiv) in THF (45 mL) was treated with NaHCO₃ (2.13 g, 25.374 mmol, 2 equiv) in H₂O (15 mL, 832.639 mmol, 65.63 equiv) for 10 min at room temperature under nitrogen atmosphere followed by the addition of benzyl chloroformate (3.25 g, 19.030 mmol, 1.5 equiv) dropwise at room temperature. The resulting mixture was stirred overnight at room temperature under nitrogen atmosphere, diluted with water (200 mL) and extracted with EtOAc (3 x100 mL). The combined organic layers were washed with brine (2×100 mL), dried over anhydrous Na₂SO₄, filtered and concentrated under reduced pressure. The residue was purified by reverse flash chromatography with the following conditions: column, C18 silica gel; mobile phase, MeCN in Water (0.1% FA), 0% to 100% gradient in 20 min; detector, UV 254 nm. This resulted in tert-butyl((1r,4r)-4-((((benzyloxy)carbonyl)amino)methyl)-4-hydroxycyclohexyl)carbamate 3 (2.8 g, 58.31%) as off-white solid.

LCMS: 379.1 (M+H). Method: Mobile Phase A: Mobile Phase A: Water/0.1% FA. Mobile Phase B: Acetonitrile/0.1% FA. Flow rate: 1.5 mL/min. COLUMN: HPH-C18 (50×3.0) mm, 2.7μ. Rt (min): 0.757; Area % —100.0.

### Step-3: benzyl (((1r,4r)-4-amino-1-hydroxycyclohexyl)methyl)carbamate

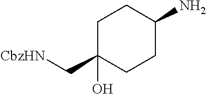 [SMILES: N[C@H]1CC[C@@](O)(CNC(=O)OCc2ccccc2)CC1]

To a stirred solution of tert-butyl((1r,4r)-4-((((benzyloxy)carbonyl)amino)methyl)-4-hydroxycyclohexyl)carbamate 3 (1 g, 2.642 mmol, 1 equiv) in DCM (20 mL) was added TFA (2.00 mL, 26.922 mmol, 10.19 equiv) dropwise at 0° C. under nitrogen atmosphere. The resulting mixture was stirred for 3 h at room temperature under nitrogen atmosphere, and concentrated under reduced pressure. The residue was purified by reverse flash chromatography with the following conditions: column, C18 silica gel; mobile phase, MeCN in Water (0.1% NH₃·H₂O), 0% to 100% gradient in 20 min; detector, UV 254 nm. This resulted in benzyl (((1r,4r)-4-amino-1-hydroxycyclohexyl)methyl)carbamate 4 (690 mg, 93.82%) as off-white solid.

LCMS: 279.1 (M+H). Method: Mobile Phase A: Water/0.1% FA. Mobile Phase B: Acetonitrile/0.1% FA. Flow rate: 1.5 mL/min. COLUMN: XB-C18 (30×3.0) mm, 1.7μ. Rt (min): 0.458; Area %—100.0.

### Step-4: benzyl (((1r,4r)-4-((2-(2,6-bis(benzyloxy)pyridin-3-yl)-1-oxoisoindolin-4-yl)amino)-1-hydroxycyclohexyl)methyl)carbamate

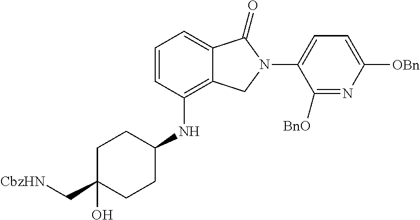 [SMILES: O=C(NC[C@]1(O)CC[C@H](Nc2cccc3c2CN(c2ccc(OCc4ccccc4)nc2OCc2ccccc2)C3=O)CC1)OCc1ccccc1]

To a stirred mixture of benzyl (((1r,4r)-4-amino-1-hydroxycyclohexyl)methyl)carbamate 4 (500 mg, 1.796 mmol, 1 equiv) and 2-[2,6-bis(benzyloxy)pyridin-3-yl]-4-bromo-3H-isoindol-1-one 5 (900.62 mg, 1.796 mmol, 1 equiv) in dioxane (10 mL), was add Pd₂(dba)₃·CHCl₃ (185.93 mg, 0.180 mmol, 0.1 equiv), CPhos (156.86 mg, 0.359 mmol, 0.2 equiv) and K₂CO₃ (744.77 mg, 5.388 mmol, 3 equiv). The resulting mixture was stirred overnight at 80° C. under nitrogen atmosphere, and filtered. The filter cake was washed with 1,4-dioxane (3×5 mL). The filtrate was concentrated under reduced pressure. The residue was purified by reverse flash chromatography with the following conditions: column, C18 silica gel; mobile phase, MeCN in Water (0.1% FA), 0% to 100% gradient in 20 min; detector, UV 254 nm. This resulted in benzyl (((1r,4r)-4-((2-(2,6-bis(benzyloxy)pyridin-3-yl)-1-oxoisoindolin-4-yl)amino)-1-hydroxycyclohexyl)methyl)carbamate 6 (400 mg, 31.87%) as yellow solid.

LCMS: 699.1 (M+H). Method: Mobile Phase A: Water/0.1% FA. Mobile Phase B: Acetonitrile/0.1% FA. Flow rate: 1.5 mL/min. COLUMN: XB-C18 (30×3.0) mm, 1.7μ. Rt (min): 1.187; Area %—100.0.

### Step-5:benzyl (((1r,4r)-4-((2-(2,6-bis(benzyloxy)pyridin-3-yl)-1-oxoisoindolin-4-yl)(2-cyclopropylethyl)amino)-1-hydroxycyclohexyl)methyl)carbamate

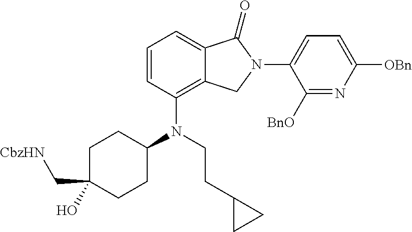 [SMILES: O=C(NC[C@]1(O)CC[C@H](N(CCC2CC2)c2cccc3c2CN(c2ccc(OCc4ccccc4)nc2OCc2ccccc2)C3=O)CC1)OCc1ccccc1]

A solution of benzyl (((1r,4r)-4-((2-(2,6-bis(benzyloxy)pyridin-3-yl)-1-oxoisoindolin-4-yl)amino)-1-hydroxycyclohexyl)methyl)carbamate 6 (400 mg, 0.572 mmol, 1 equiv), 2-cyclopropylacetaldehyde 7 (240.74 mg, 2.860 mmol, 5 equiv), TFA (195.80 mg, 1.716 mmol, 3 equiv) in DCM (10 mL) and DMF (2.4 mL) was stirred for 2 h at room temperature under nitrogen atmosphere followed by the addition of STAB (606.57 mg, 2.860 mmol, 5 equiv) dropwise at 0° C. The resulting mixture was stirred overnight at room temperature under nitrogen atmosphere, concentrated under vacuum, and purified by reverse flash chromatography with the following conditions: column, C18 silica gel; mobile phase, MeCN in Water (0.1% FA), 0% to 100% gradient in 10 min; detector, UV 254 nm. This resulted in benzyl (((1r,4r)-4-((2-(2,6-bis(benzyloxy)pyridin-3-yl)-1-oxoisoindolin-4-yl)(2-cyclopropylethyl)amino)-1-hydroxycyclohexyl)methyl)carbamate 8 (270 mg, 61.50%) as off-white solid.

LCMS: 767.1.1 (M+H). Method: Mobile Phase A: Water/0.1% FA. Mobile Phase B: Acetonitrile/0.1% FA. Flow rate: 1.5 mL/min. COLUMN: XB-C18 (30×3.0) mm, 1.7μ. Rt (min): 1.198; Area %—100.0.

### Step-6:3-(4-(((1r,4r)-4-(aminomethyl)-4-hydroxycyclohexyl)(2-cyclopropylethyl)amino)-1-oxoisoindolin-2-yl)piperidine-2,6-dione

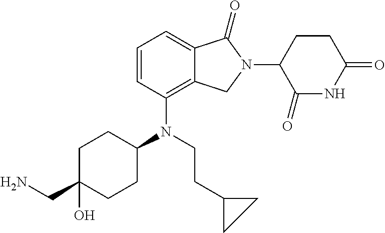 [SMILES: NC[C@]1(O)CC[C@H](N(CCC2CC2)c2cccc3c2CN(C2CCC(=O)NC2=O)C3=O)CC1]

To a solution of benzyl (((1r,4r)-4-((2-(2,6-bis(benzyloxy)pyridin-3-yl)-1-oxoisoindolin-4-yl)(2-cyclopropylethyl)amino)-1-hydroxycyclohexyl)methyl)carbamate 8 (200 mg, 0.261 mmol, 1 equiv) in 4 mL DMF was added Pd/C (100 mg) in a pressure tank. The mixture was hydrogenated at room temperature under 5 psi of hydrogen pressure for overnight, filtered through a Celite pad and concentrated under reduced pressure. The residue was purified by prep-HPLC with the following conditions: Column: Sunfire prep C18 column, 30×150 mm, 5 m; Mobile Phase A: Water (0.1% FA), Mobile Phase B: ACN; Flow rate: 60 mL/min; Gradient: 18% B to 40% B in 7 min, 40% B; Wave Length: 254/220 nm; RT1 (min): 3.17. This resulted in 3-(4-(((1r,4r)-4-(aminomethyl)-4-hydroxycyclohexyl)(2-cyclopropylethyl)amino)-1-oxoisoindolin-2-yl)piperidine-2,6-dione (39.4 mg, 33.24%) as off-white solid.

¹H-NMR (400 MHz, DMSO-d₆): δ 8.46 (s, 1H), 7.47 (t, J=7.7 Hz, 1H), 7.32 (dd, J=12.2, 7.6 Hz, 2H), 5.15 (dd, J=13.2, 5.1 Hz, 1H), 4.47-4.26 (m, 2H), 3.29 (hept, J=7.0 Hz, 2H), 3.13 (td, J=12.3, 11.9, 5.9 Hz, 1H), 2.96 (ddd, J=17.8, 13.5, 5.4 Hz, 1H), 2.71-2.58 (m, 3H), 2.53 (dd, J=13.0, 4.5 Hz, 1H), 2.07 (tt, J=7.2, 4.2 Hz, 1H), 1.91 (td, J=12.3, 6.5 Hz, 2H), 1.77-1.52 (m, 4H), 1.46-1.16 (m, 4H), 0.72 (qq, J=7.3, 4.9, 3.7 Hz, 1H), 0.40 (dt, J=8.6, 2.7 Hz, 2H). LCMS: 441.25 (M+H). Method: Mobile Phase A: Water/5 mM NH₄HCO₃. Mobile Phase B: Acetonitrile. Flow rate: 1.5 mL/min. COLUMN: Kinetex EVO C18 (50x 3.0) mm, 2.7μ. Rt (min): 1.153; Area % —94.66.

### Compound 530

### 3-(4-(((1r,4r)-4-(aminomethyl)-4-hydroxycyclohexyl)(4,4,4-trifluorobutyl)amino)-1-oxoisoindolin-2-yl)piperidine-2,6-dione

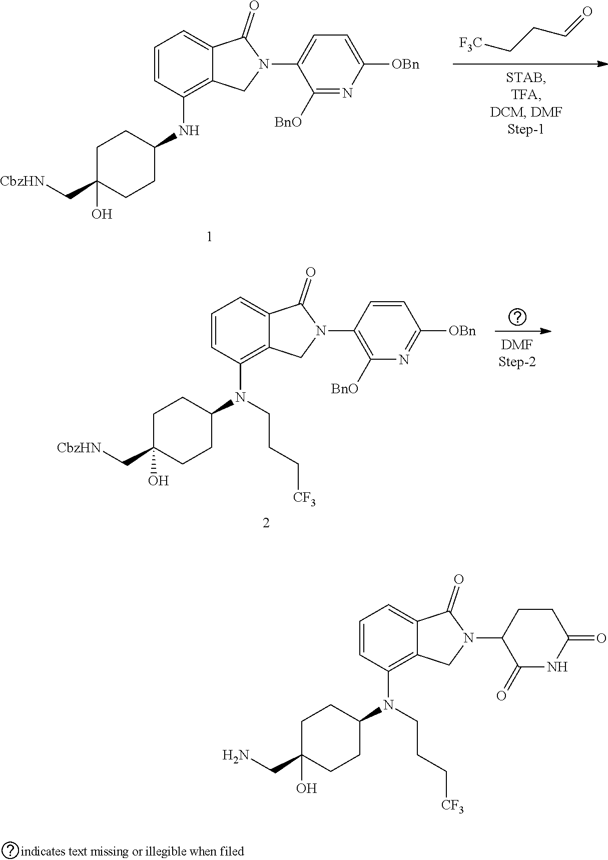 [SMILES: O=C(NC[C@]1(O)CC[C@H](Nc2cccc3c2CN(c2ccc(OCc4ccccc4)nc2OCc2ccccc2)C3=O)CC1)OCc1ccccc1 ; O=CCCC(F)(F)F ; O=C(NC[C@]1(O)CC[C@H](N(CCCC(F)(F)F)c2cccc3c2CN(c2ccc(OCc4ccccc4)nc2OCc2ccccc2)C3=O)CC1)OCc1ccccc1 ; NC[C@]1(O)CC[C@H](N(CCCC(F)(F)F)c2cccc3c2CN(C2CCC(=O)NC2=O)C3=O)CC1]

### Step-1:benzyl (((1r,4r)-4-((2-(2,6-bis(benzyloxy)pyridin-3-yl)-1-oxoisoindolin-4-yl)(4,4,4-trifluorobutyl)amino)-1-hydroxycyclohexyl)methyl)carbamate

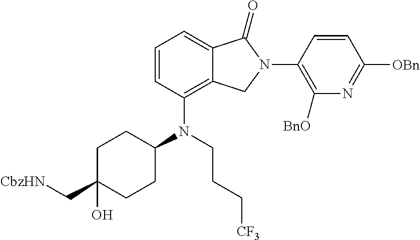 [SMILES: O=C(NC[C@]1(O)CC[C@H](N(CCCC(F)(F)F)c2cccc3c2CN(c2ccc(OCc4ccccc4)nc2OCc2ccccc2)C3=O)CC1)OCc1ccccc1]

A solution of benzyl (((1r,4r)-4-((2-(2,6-bis(benzyloxy)pyridin-3-yl)-1-oxoisoindolin-4-yl)amino)-1-hydroxycyclohexyl)methyl)carbamate 1 (200 mg, 0.286 mmol, 1 equiv), 4,4,4-trifluorobutanal (180.42 mg, 1.430 mmol, 5 equiv) and TFA (97.90 mg, 0.858 mmol, 3 equiv) in DCM (5 mL) and DMF (1.2 mL) was stirred for 2 h at room temperature under nitrogen atmosphere followed by the addition of STAB (303.28 mg, 1.430 mmol, 5 equiv) dropwise at 0° C. The resulting mixture was further stirred overnight at room temperature under nitrogen atmosphere, concentrated under vacuum, and purified by reverse flash chromatography with the following conditions: column, C18 silica gel; mobile phase, MeCN in Water (0.1% FA), 0% to 100% gradient in 20 min; detector, UV 254 nm. This resulted in benzyl (((1r,4r)-4-((2-(2,6-bis(benzyloxy)pyridin-3-yl)-1-oxoisoindolin-4-yl)(4,4,4-trifluorobutyl)amino)-1-hydroxycyclohexyl)methyl)carbamate 2 (150 mg, 64.79%) as off-white solid.

LCMS: 809.1 (M+H). Method: Mobile Phase A: Water/0.100 FA. Mobile Phase B: Acetonitrile/0.1% FA. Flow rate: 1.5 mL/min. COLUMN: XB-C18 (30 x3.0) mm, 1.7μ. Rt (min): 1.273; Area %—100.0.

### Step-2:3-(4-(((1r,4r)-4-(aminomethyl)-4-hydroxycyclohexyl)(4,4,4-trifluorobutyl)amino)-1-oxoisoindolin-2-yl)piperidine-2,6-dione

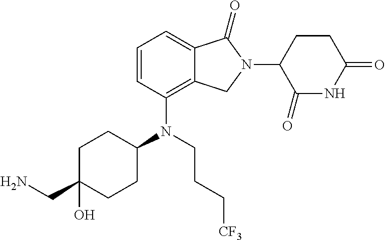 [SMILES: NC[C@]1(O)CC[C@H](N(CCCC(F)(F)F)c2cccc3c2CN(C2CCC(=O)NC2=O)C3=O)CC1]

To a solution of benzyl (((1r,4r)-4-((2-(2,6-bis(benzyloxy)pyridin-3-yl)-1-oxoisoindolin-4-yl)(4,4,4-trifluorobutyl)amino)-1-hydroxycyclohexyl)methyl)carbamate 2 (120 mg, 0.148 mmol, 1 equiv) in 3 mL DMF was added Pd/C (100 mg) under nitrogen atmosphere in a 25 mL round-bottom flask. The mixture was hydrogenated at room temperature for overnight under hydrogen atmosphere using a hydrogen balloon, filtered through a Celite pad and concentrated under reduced pressure. The residue was purified by reverse flash chromatography with the following conditions: column, C18 silica gel; mobile phase, MeCN in Water (0.1% FA), 0% to 100% gradient in 20 min; detector, UV 254 nm. This resulted in 3-(4-(((1r,4r)-4-(aminomethyl)-4-hydroxycyclohexyl)(4,4,4-trifluorobutyl)amino)-1-oxoisoindolin-2-yl)piperidine-2,6-dione (18.6 mg, 25.25%) as off-white solid.

¹H-NMR (400 MHz, DMSO-d₆): δ 8.36 (s, 1H), 7.44 (t, J=7.6 Hz, 1H), 7.34 (dd, J=7.6, 2.4 Hz, 2H), 5.09 (dd, J=13.1, 5.1 Hz, 1H), 4.42-4.15 (m, 3H), 3.31-3.11 (m, 2H), 3.08-2.80 (m, 2H), 2.68-2.52 (m, 2H), 2.46 (d, J=4.6 Hz, 1H), 2.23 (tq, J=10.9, 6.4 Hz, 2H), 2.00 (dd, J=12.3, 6.5 Hz, 1H), 1.79 (s, 2H), 1.69-1.38 (m, 6H), 1.25 (dd, J=17.4, 8.6 Hz, 2H). LCMS: 441.25 (M+H). Method: Mobile Phase A: Water/0.1% FA. Mobile Phase B: Acetonitrile/0.1% FA. Flow rate: 1.5 mL/min. COLUMN: XB-C18 (30×3.0) mm, 1.7μ. Rt (min): 1.070; Area %—96.67.

### Compound 532

The compound having the following structure is prepared in analogy from the preparation procedure described for the above-mentioned compounds.

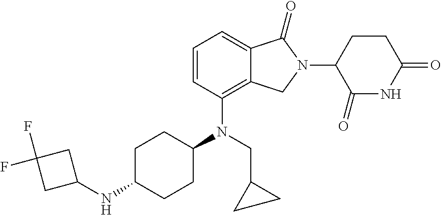 [SMILES: O=C1CCC(N2Cc3c(cccc3N(CC3CC3)[C@H]3CC[C@H](NC4CC(F)(F)C4)CC3)C2=O)C(=O)N1]

### Compound 533

### 3-(4-((cyclopropylmethyl)((1r,4r)-4-((3,3,3-trifluoropropyl)amino)cyclohexyl)amino)-1-oxoisoindolin-2-yl)piperidine-2,6-dione

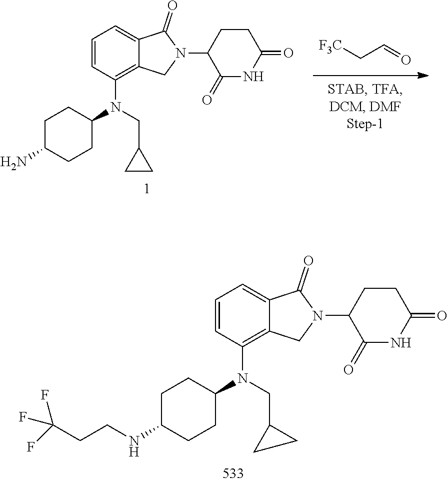 [SMILES: N[C@H]1CC[C@H](N(CC2CC2)c2cccc3c2CN(C2CCC(=O)NC2=O)C3=O)CC1 ; O=CCC(F)(F)F ; O=C1CCC(N2Cc3c(cccc3N(CC3CC3)[C@H]3CC[C@H](NCCC(F)(F)F)CC3)C2=O)C(=O)N1]

### Step-1:3-(4-((cyclopropylmethyl)((1r,4r)-4-((3,3,3-trifluoropropyl)amino)cyclohexyl)amino)-1-oxoisoindolin-2-yl)piperidine-2,6-dione

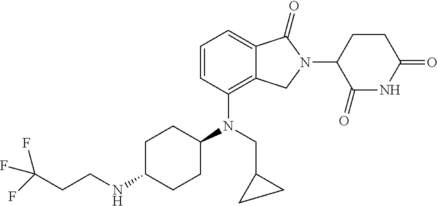 [SMILES: O=C1CCC(N2Cc3c(cccc3N(CC3CC3)[C@H]3CC[C@H](NCCC(F)(F)F)CC3)C2=O)C(=O)N1]

A solution of 3-(4-(((1r,4r)-4-aminocyclohexyl)(cyclopropylmethyl)amino)-1-oxoisoindolin-2-yl)piperidine-2,6-dione 1 (150 mg, 0.365 mmol, 1 equiv) and 3,3,3-trifluoropropanal (204.71 mg, 1.825 mmol, 5 equiv) in DCM (2.75 mL, 43.259 mmol) and DMF (0.7 mL, 9.045 mmol) was treated with TFA (124.99 mg, 1.095 mmol, 3 equiv) for 2 h at 0 degrees C. under nitrogen atmosphere followed by the addition of STAB (387.21 mg, 1.825 mmol, 5 equiv) dropwise at 0 degrees C. The resulting mixture was stirred for additional 16 h at room temperature, concentrated under vacuum, and purified by reverse flash chromatography with the following conditions: column, C18 silica gel; mobile phase, MeCN in Water (0.1% FA), 0% to 100% gradient in 10 min; detector, UV 254 nm. This resulted in 3-(4-((cyclopropylmethyl)((1r,4r)-4-((3,3,3-trifluoropropyl)amino)cyclohexyl)amino)-1-oxoisoindolin-2-yl)piperidine-2,6-dione (8.6 mg, 4.65%) as off-white solid.

¹H-NMR (400 MHz, Methanol-d₄): δ 7.55-7.43 (m, 3H), 5.14 (dd, J=13.3, 5.2 Hz, 1H), 4.48 (s, 2H), 3.14-2.84 (m, 6H), 2.83-2.74 (m, 1H), 2.71-2.51 (m, 2H), 2.51-2.39 (m, 2H), 2.24-2.14 (m, 1H), 2.09-1.85 (m, 4H), 1.53-1.38 (m, 3H), 1.31-1.21 (m, 2H), 0.73 (d, J=5.4 Hz, 1H), 0.32 (dt, J=8.1, 2.8 Hz, 2H). LCMS: 557.2 (M+H). Method: Mobile Phase A: Water/0.1% FA. Mobile Phase B: Acetonitrile/0.1% FA. Flow rate: 1.5 mL/min. COLUMN: XB-C18 (30×3.0) mm, 1.7μ. Rt (min): 1.010; Area %—94.49.

### Compound 534

### 3-(4-((cyclopropylmethyl)((1r,4r)-4-(oxetan-3-ylamino)cyclohexyl)amino)-1-oxoisoindolin-2-yl)piperidine-2,6-dione

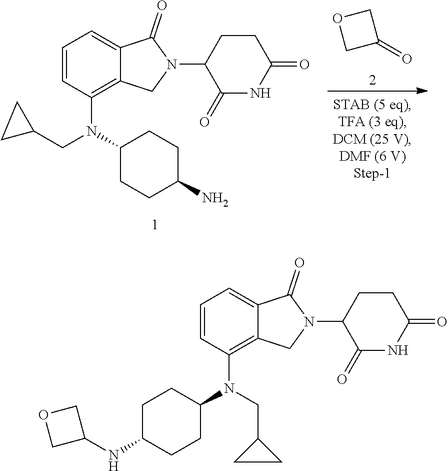

### Step-1:3-(4-((cyclopropylmethyl)((1r,4r)-4-(oxetan-3-ylamino)cyclohexyl)amino)-1-oxoisoindolin-2-yl)piperidine-2,6-dione

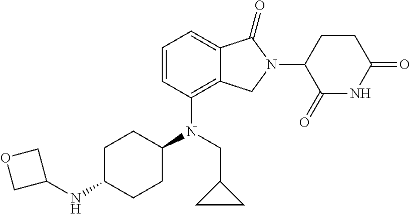 [SMILES: O=C1CCC(N2Cc3c(cccc3N(CC3CC3)[C@H]3CC[C@H](NC4COC4)CC3)C2=O)C(=O)N1]

To a stirred solution of 3-{4-[(cyclopropylmethyl)[(1r,4r)-4-aminocyclohexyl]amino]-1-oxo-3H-isoindol-2-yl}piperidine-2,6-dione 1 (Compound 235, 100 mg, 0.244 mmol, 1 equiv) and 3-oxetanone 2 (87.77 mg, 1.220 mmol, 5 equiv) in DCM (3.2 mL, 50.338 mmol, 206.65 equiv) and DMF (0.8 mL, 10.337 mmol, 42.44 equiv) was added TFA (83.33 mg, 0.732 mmol, 3 equiv) dropwise at room temperature under nitrogen atmosphere. The resulting mixture was stirred for 2 h at room temperature under nitrogen atmosphere. To the above mixture was added STAB (258.14 mg, 1.220 mmol, 5 equiv) in portions over 2 min at 0° C. The resulting mixture was stirred for additional overnight at room temperature. The resulting mixture was extracted with EtOAc (3 x100 mL). The combined organic layers were washed with brine (3×50 mL), dried over anhydrous Na₂SO₄. filtered and concentrated. The crude product (100 mg) was purified by Prep-HPLC with the following conditions (Column: YMC-Actus Triart C18, 30*150 mm, 5 μm; Mobile Phase A: Water (0.1% FA), Mobile Phase B: ACN; Flow rate: 60 mL/min; Gradient: 8% B to 26% B in 7 min, 26% B; Wave Length: 254/220 nm; RT1 (min): 5.55; Number Of Runs: 0) to afford 3-{4-[(cyclopropylmethyl)[(1r,4r)-4-(oxetan-3-ylamino)cyclohexyl]amino]-1-oxo-3H-isoindol-2-yl}piperidine-2,6-dione (28 mg, 24.64%) as white solid.

¹H-NMR (400 MHz, DMSO-d₆): δ 10.94 (s, 1H), 8.13 (d, J=6.2 Hz, 1H), 7.40 (t, J=7.6 Hz, 1H), 7.36 (s, 2H), 5.08 (dd, J=13.2, 5.2 Hz, 1H), 4.59 (t, J=6.7 Hz, 2H), 4.46-4.18 (m, 4H), 4.00 (dd, J=13.8, 7.1 Hz, 1H), 3.12-2.78 (m, 4H), 2.57 (dt, J=17.4, 2.8 Hz, 2H), 2.43 (d, J=4.4 Hz, 1H), 1.99 (dd, J=8.8, 3.6 Hz, 1H), 1.73 (d, J=11.8 Hz, 4H), 1.32 (s, 2H), 1.09 (s, 2H), 0.66 (d, J=5.3 Hz, 1H), 0.34-0.18 (m, 2H), 0.08-−0.09 (m, 2H).LCMS:467.2 (M+H). Method: Mobile Phase A: 0.05% TFA in H₂O. Mobile Phase B: Acetonitrile. Flow rate: 1.8 mL/min. COLUMN: Astinces Express C18 (30×3.0) mm, 2.6μ. Rt (min): 0.799; Area %—99.25.

### Compound 535

### 3-(4-(((1R,3R,4R)-4-amino-3-fluorocyclohexyl)(cyclopropylmethyl)amino)-1-oxoisoindolin-2-yl)piperidine-2,6-dione; formic acid

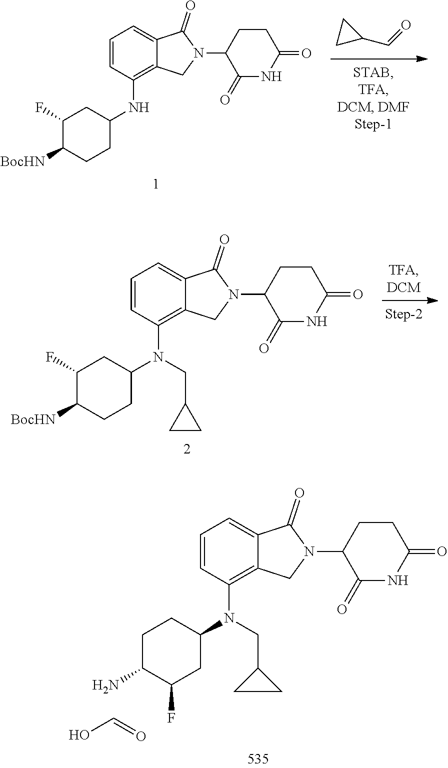

### Step-1: tert-butyl ((1R,2R,4R)-4-((cyclopropylmethyl)(2-(2,6-dioxopiperidin-3-yl)-1-oxoisoindolin-4-yl)amino)-2-fluorocyclohexyl)carbamate

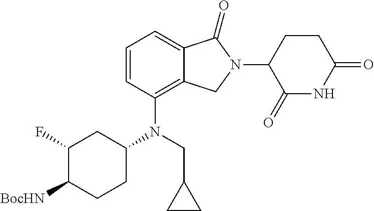 [SMILES: CC(C)(C)OC(=O)N[C@@H]1CC[C@@H](N(CC2CC2)c2cccc3c2CN(C2CCC(=O)NC2=O)C3=O)C[C@H]1F]

A solution of tert-butyl ((1R,2R,4R)-4-((2-(2,6-dioxopiperidin-3-yl)-1-oxoisoindolin-4-yl)amino)-2-fluorocyclohexyl)carbamate 1 (150 mg, 0.316 mmol, 1 equiv), cyclopropanecarbaldehyde (110.78 mg, 1.580 mmol, 5 equiv) and TFA (108.13 mg, 0.948 mmol, 3 equiv) in DCM (3.75 mL) and DMF (0.9 mL) was stirred for 2 h at room temperature under nitrogen atmosphere followed by the addition of STAB (334.97 mg, 1.580 mmol, 5 equiv) dropwise at 0° C. The resulting mixture was stirred for overnight at room temperature under nitrogen atmosphere. The reaction was quenched with water at room temperature. The resulting mixture was extracted with CH₂Cl₂ (3×20 mL). The combined organic layers were washed with brine (2×20 mL), dried over anhydrous Na₂SO₄, filtered, and concentrated under reduced pressure. The residue was purified by reverse flash chromatography with the following conditions (column, C18 silica gel; mobile phase, MeCN in Water (0.1% FA), 0% to 100% gradient in 20 min; detector, UV 254 nm) to afford tert-butyl ((1R,2R,4R)-4-((cyclopropylmethyl)(2-(2,6-dioxopiperidin-3-yl)-1-oxoisoindolin-4-yl)amino)-2-fluorocyclohexyl)carbamate 2 (53 mg, 31.72%) as off-white solid.

LCMS: 529.1 (M+H). Method: Mobile Phase A: Mobile Phase A: Water/0.1% FA. Mobile Phase B: Acetonitrile/0.1% FA. Flow rate: 1.5 mL/min. COLUMN: HPH-C18 (50×3.0) mm, 2.7μ. Rt (min): 0.931; Area % —100.0.

### Step-2:3-(4-(((1R,3R,4R)-4-amino-3-fluorocyclohexyl)(cyclopropylmethyl)amino)-1-oxoisoindolin-2-yl)piperidine-2,6-dione; formic acid

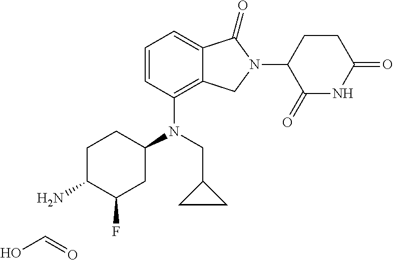

To a stirred solution of tert-butyl ((1R,2R,4R)-4-((cyclopropylmethyl)(2-(2,6-dioxopiperidin-3-yl)-1-oxoisoindolin-4-yl)amino)-2-fluorocyclohexyl)carbamate 2 (43 mg, 0.079 mmol, 1 equiv) in DCM (3 mL, 47.192 mmol, 580.16 equiv) was added TFA (0.6 mL, 8.078 mmol, 99.31 equiv) dropwise at 0° C. under nitrogen atmosphere. The resulting mixture was stirred for 3 h at room temperature under nitrogen atmosphere, concentrated under vacuum and purified by reverse flash chromatography with the following conditions: column, C18 silica gel; mobile phase, MeCN in Water (0.1% FA), 0% to 100% gradient in 10 min; detector, UV 254 nm. This resulted in 3-(4-(((1R,3R,4R)-4-amino-3-fluorocyclohexyl)(cyclopropylmethyl)amino)-1-oxoisoindolin-2-yl)piperidine-2,6-dione; formic acid (17.9 mg, 51.05%) as a yellow solid.

¹H-NMR (400 MHz, DMSO-d₆): δ 10.93 (s, 1H), 8.18 (d, J=5.0 Hz, 3H), 7.45-7.30 (m, 3H), 5.07 (dd, J=13.2, 5.1 Hz, 1H), 4.67-4.15 (m, 4H), 3.14 (d, J=12.1 Hz, 2H), 3.02 (ddd, J=14.3, 6.3, 2.6 Hz, 1H), 2.96-2.78 (m, 2H), 2.59-2.46 (m, 2H), 2.20 (s, 1H), 2.02-1.81 (m, 2H), 1.75-1.62 (m, 2H), 1.46 (tt, J=14.0, 7.4 Hz, 1H), 1.24 (d, J=40.1 Hz, 1H), 0.64 (q, J=6.5 Hz, 1H), 0.25 (dq, J=6.8, 3.5 Hz, 2H), −0.05 (s, 2H). LCMS: 429.25 (M+H). Method: Mobile Phase A: Water/5 mM NH₄HCO₃. Mobile Phase B: Acetonitrile. Flow rate: 1.5 mL/min. COLUMN: Kinetex EVO C18 (50×3.0) mm, 2.7μ. Rt (min): 1.287; Area %—95.27.

### Compound 536

### 3-(4-(((1r,4r)-4-(aminomethyl)-4-hydroxycyclohexyl)(cyclopropylmethyl)amino)-1-oxoisoindolin-2-yl)piperidine-2,6-dione

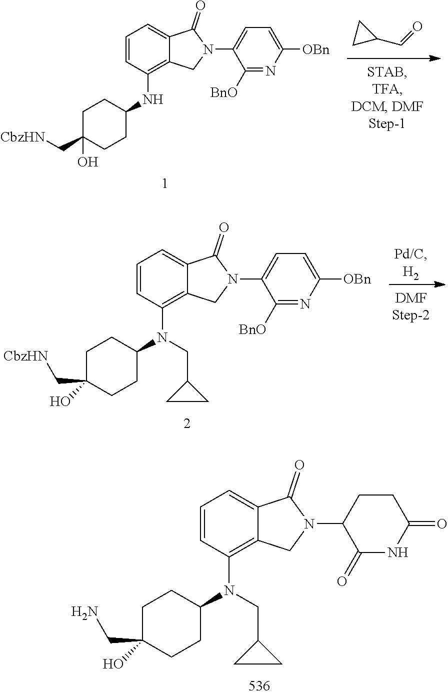 [SMILES: O=C(NC[C@]1(O)CC[C@H](Nc2cccc3c2CN(c2ccc(OCc4ccccc4)nc2OCc2ccccc2)C3=O)CC1)OCc1ccccc1 ; O=CC1CC1 ; O=C(NC[C@]1(O)CC[C@H](N(CC2CC2)c2cccc3c2CN(c2ccc(OCc4ccccc4)nc2OCc2ccccc2)C3=O)CC1)OCc1ccccc1 ; NC[C@]1(O)CC[C@H](N(CC2CC2)c2cccc3c2CN(C2CCC(=O)NC2=O)C3=O)CC1]

### Step-1: benzyl (((1r,4r)-4-((2-(2,6-bis(benzyloxy)pyridin-3-yl)-1-oxoisoindolin-4-yl)(cyclopropylmethyl)amino)-1-hydroxycyclohexyl)methyl)carbamate

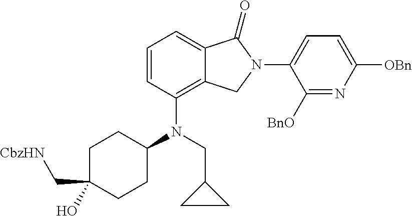 [SMILES: O=C(NC[C@]1(O)CC[C@H](N(CC2CC2)c2cccc3c2CN(c2ccc(OCc4ccccc4)nc2OCc2ccccc2)C3=O)CC1)OCc1ccccc1]

A solution of benzyl (((1r,4r)-4-((2-(2,6-bis(benzyloxy)pyridin-3-yl)-1-oxoisoindolin-4-yl)amino)-1-hydroxycyclohexyl)methyl)carbamate 1 (400 mg, 0.572 mmol, 1 equiv), cyclopropanecarbaldehyde (200.60 mg, 2.860 mmol, 5 equiv), TFA (195.80 mg, 1.716 mmol, 3 equiv) in DCM (10 mL) and DMF (2.4 mL) was stirred for 2 h at room temperature under nitrogen atmosphere followed by the addition of STAB (606.57 mg, 2.860 mmol, 5 equiv) dropwise at 0° C. The resulting mixture was stirred overnight at room temperature under nitrogen atmosphere. The resulting mixture was concentrated under reduced pressure. The residue was purified by reverse flash chromatography with the following conditions: column, C18 silica gel; mobile phase, MeCN in Water (0.1% FA), 0% to 100% gradient in 20 min; detector, UV 254 nm. This resulted in benzyl (((1r,4r)-4-((2-(2,6-bis(benzyloxy)pyridin-3-yl)-1-oxoisoindolin-4-yl)(cyclopropylmethyl)amino)-1-hydroxycyclohexyl)methyl)carbamate 2 (237 mg, 54.99%) as a yellow solid.

LCMS: 753.1 (M+H). Method: Mobile Phase A: Water/0.1% FA. Mobile Phase B: Acetonitrile/0.1% FA. Flow rate: 1.5 mL/min. COLUMN: XB-C18 (30×3.0) mm, 1.7μ. Rt (min): 1.242; Area %—100.0.

### Step-2: 3-(4-(((1r,4r)-4-(aminomethyl)-4-hydroxycyclohexyl)(cyclopropylmethyl)amino)-1-oxoisoindolin-2-yl)piperidine-2,6-dione

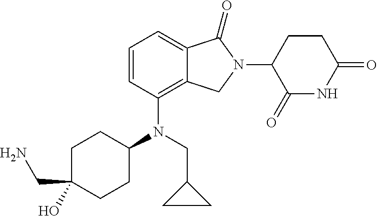 [SMILES: NC[C@]1(O)CC[C@H](N(CC2CC2)c2cccc3c2CN(C2CCC(=O)NC2=O)C3=O)CC1]

To a solution of benzyl (((1r,4r)-4-((2-(2,6-bis(benzyloxy)pyridin-3-yl)-1-oxoisoindolin-4-yl)(cyclopropylmethyl)amino)-1-hydroxycyclohexyl)methyl)carbamate 2 (170 mg, 0.226 mmol, 1 equiv) in 3 mL DMF was added Pd/C (100 mg) under nitrogen atmosphere in a 25 mL round-bottom flask. The mixture was hydrogenated at room temperature for 3 h under hydrogen atmosphere using a hydrogen balloon, filtered through a Celite pad and concentrated under reduced pressure. The residue was purified by reverse flash chromatography with the following conditions: column, C18 silica gel; mobile phase, MeCN in Water (0.1% FA), 0% to 100% gradient in 20 min; detector, UV 254 nm. This resulted in 3-(4-(((1r,4r)-4-(aminomethyl)-4-hydroxycyclohexyl)(cyclopropylmethyl)amino)-1-oxoisoindolin-2-yl)piperidine-2,6-dione (27.3 mg, 27.45%) as off-white solid.

¹H-NMR (400 MHz, Methanol-d₄): δ 8.57 (s, 1H), 7.49 (h, J=3.8, 3.3 Hz, 3H), 5.14 (dd, J=13.3, 5.2 Hz, 1H), 4.49 (s, 2H), 3.06 (dtd, J=19.6, 13.7, 13.2, 5.5 Hz, 3H), 2.91 (ddd, J=18.4, 13.5, 5.3 Hz, 1H), 2.80 (s, 3H), 2.54 (qd, J=13.2, 4.7 Hz, 1H), 2.24-2.13 (m, 1H), 1.88-1.63 (m, 6H), 1.40 (td, J=12.3, 7.4 Hz, 2H), 0.75 (dh, J=11.6, 6.2 Hz, 1H), 0.38-0.29 (m, 2H).

LCMS: 441.25 (M+H). Method: Mobile Phase A: Water/5 mM NH₄HCO₃. Mobile Phase B: Acetonitrile. Flow rate: 1.5 mL/min. COLUMN: Kinetex EVO C18 (50×3.0) mm, 2.7μ. Rt (min): 1.007; Area %—97.92.

### Compound 539 and 539A

### 3-{4-[(2-cyclopropylethyl)[(1s,4s)-4-[(2,2-difluoroethyl)amino]cyclohexyl]amino]-1-oxo-3H-isoindol-2-yl}piperidine-2,6-dione and 3-{4-[(2-cyclopropylethyl)[(1r,4r)-4-[(2,2-difluoroethyl)amino]cyclohexyl]amino]-1-oxo-3H-isoindol-2-yl}piperidine-2,6-dione

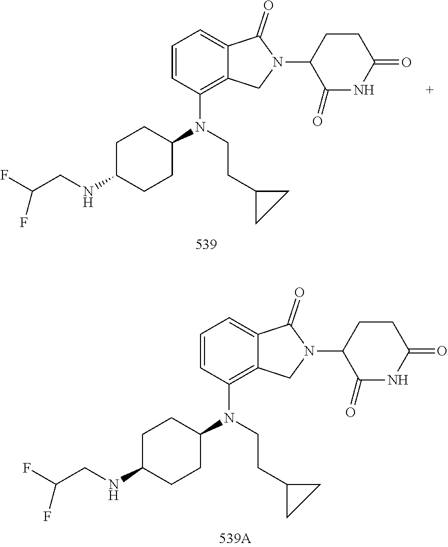 [SMILES: O=C1CCC(N2Cc3c(cccc3N(CCC3CC3)[C@H]3CC[C@H](NCC(F)F)CC3)C2=O)C(=O)N1 ; O=C1CCC(N2Cc3c(cccc3N(CCC3CC3)[C@H]3CC[C@@H](NCC(F)F)CC3)C2=O)C(=O)N1]

The compounds were prepared by the general procedure described in the preparation of Compounds 515 and 515A using the appropriate amine.

Compound 539: LCMS: 489.26 (M+H). ¹H NMR (400 MHz, DMSO-d₆): 11.07 (s, 1H), 9.15 (s, 2H), 7.50 (s, 1H), 7.44 (d, J=7.6 Hz, 2H), 6.51 (t, J=3.9 Hz, 1H), 5.18 (d, J=5.2 Hz, 1H), 4.51 (s, 1H), 4.37 (d, J=17.3 Hz, 1H), 3.65 (s, 2H), 3.43 (s, 1H), 3.26 (t, J=6.4 Hz, 3H), 3.01-2.96 (m, 1H), 2.69 (d, J=10.4 Hz, 1H), 2.52 (d, J=4.6 Hz, 1H), 2.13-2.10 (m, 1H), 1.93 (s, 4H), 1.79 (s, 2H), 1.60 (s, 2H), 1.26 (s, 2H), 0.66 (d, J=7.0 Hz, 1H), 0.42-0.40 (m, 2H).

Compound 539A: LCMS: 489.26 (M+H). ¹H NMR (400 MHz, DMSO-d₆): 10.98 (s, 1H), 9.07 (s, 2H), 7.44 (d, J=7.7 Hz, 1H), 7.32-7.26 (m, 2H), 6.39 (t, J=2.6 Hz, 1H), 5.16-5.11 (m, 1H), 4.34 (d, J=17.2 Hz, 1H), 4.23 (d, J=17.2 Hz, 1H), 3.53 (s, 1H), 3.21 (d, J=7.3 Hz, 2H), 3.09 (s, 2H), 2.91 (dd, J=10.8, 6.5 Hz, 1H), 2.61 (d, J=3.9 Hz, 1H), 2.45 (s, 1H), 2.12-2.07 (m, 2H), 2.02-1.96 (m, 1H), 1.85 (s, 2H), 1.57 (d, J=13.2 Hz, 2H), 1.40 (d, J=13.1 Hz, 2H), 1.14 (dd, J=7.0, 2.4 Hz, 2H), 0.65-0.60 (m, 1H), 0.32 (q, J=3.9, 3.2 Hz, 2H), −0.06 (dd, J=5.0, 1.5 Hz, 2H).

### Compound 540

### 3-{4-[(2-cyclopropylethyl)[(1r,4r)-4-[(1,3-difluoropropan-2-yl)amino]cyclohexyl]amino]-1-oxo-3H-isoindol-2-yl}piperidine-2,6-dione

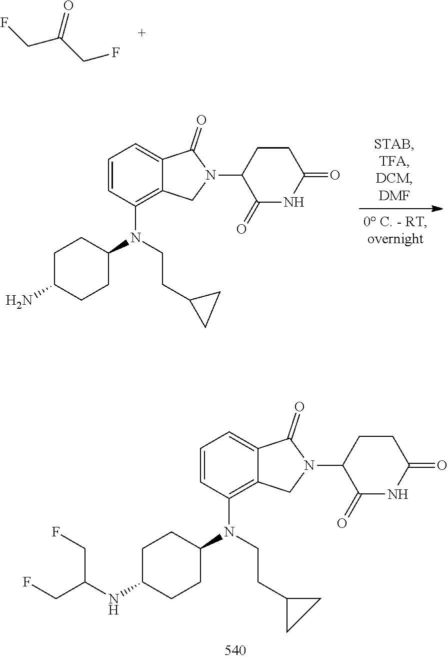 [SMILES: O=C(CF)CF ; N[C@H]1CC[C@H](N(CCC2CC2)c2cccc3c2CN(C2CCC(=O)NC2=O)C3=O)CC1 ; O=C1CCC(N2Cc3c(cccc3N(CCC3CC3)[C@H]3CC[C@H](NC(CF)CF)CC3)C2=O)C(=O)N1]

A solution of 1,3-difluoropropan-2-one (150 mg, 1.59 mmol, 5 equiv) and 3-{4-[(2-cyclopropylethyl)[(1r,4r)-4-aminocyclohexyl]amino]-1-oxo-3H-isoindol-2-yl}piperidine-2,6-dione (135 mg, 0.32 mmol, 1 equiv) in DCM (3.75 mL) and DMF (0.9 mL) was stirred for 10 min at 0° C. under nitrogen atmosphere. To the above mixture was added TFA (0.07 ml, 0.957 mmol, 3 equiv) dropwise over 10 min at 0° C. The resulting mixture was stirred for 2 h at room temperature. To the above mixture was added STAB (338 mg, 1.59 mmol, 5 equiv) in portions over 30 min at 0° C. The resulting mixture was stirred for additional overnight at room temperature. The resulting mixture was concentrated under reduced pressure. The crude product was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 18% B to 37% B in 7 min; Wave Length: 254/220 nm; RT1 (min): 4.28) to afford 3-{4-[(2-cyclopropylethyl)[(1r,4r)-4-[(1,3-difluoropropan-2-yl)amino]cyclohexyl]amino]-1-oxo-3H-isoindol-2-yl}piperidine-2,6-dione (64 mg, 39%) as an off-white solid.

LC-MS: (ES, m/z): [M+1]=503.25; ¹H NMR (400 MHz, DMSO-d₆) δ 10.97 (s, 1H), 7.41 (t, J=7.6 Hz, 1H), 7.26 (dd, J=17.8, 7.7 Hz, 2H), 5.10 (dd, J=13.2, 5.2 Hz, 1H), 4.67-4.17 (m, 6H), 3.26-3.01 (m, 4H), 2.90 (ddd, J=17.8, 13.4, 5.3 Hz, 1H), 2.59 (d, J=18.9 Hz, 2H), 2.46 (d, J=9.2 Hz, 2H), 2.06-1.86 (m, 3H), 1.76 (s, 2H), 1.56 (d, J=9.4 Hz, 2H), 1.27-1.02 (m, 4H), 0.68-0.57 (m, 1H), 0.39-0.24 (m, 2H), −0.06 (dd, J=5.0, 1.5 Hz, 2H).

### Compound 541 and 541A

### 3-(4-((2-cyclopropylethyl)((1r,4r)-4-((2-methoxy-2-methylpropyl)amino)cyclohexyl)amino)-1-oxoisoindolin-2-yl)piperidine-2,6-dione and 3-(4-((2-cyclopropylethyl)((1s,4s)-4-((2-methoxy-2-methylpropyl)amino)cyclohexyl)amino)-1-oxoisoindolin-2-yl)piperidine-2,6-dione

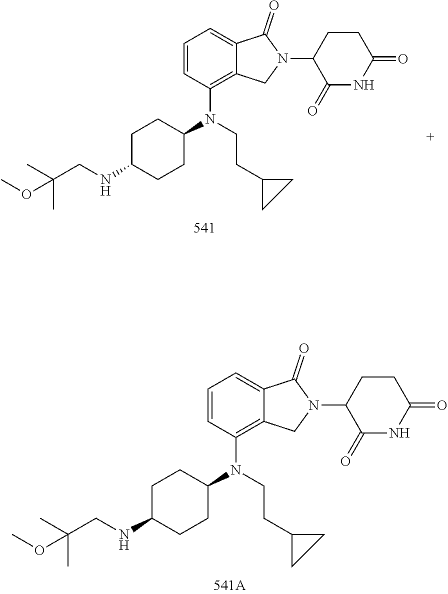 [SMILES: COC(C)(C)CN[C@H]1CC[C@H](N(CCC2CC2)c2cccc3c2CN(C2CCC(=O)NC2=O)C3=O)CC1 ; COC(C)(C)CN[C@H]1CC[C@@H](N(CCC2CC2)c2cccc3c2CN(C2CCC(=O)NC2=O)C3=O)CC1]

The compounds were prepared by the general procedure described in the preparation of Compounds 515 and 515A using the appropriate amine.

Compound 541: LCMS: 511.4 (M+H). ¹H NMR (400 MHz, DMSO-d6): δ 11.02 (s, 1H), 7.47 (t, J=7.6 Hz, 1H), 7.31 (dd, J=19.5, 7.6 Hz, 2H), 5.15 (dd, J=13.1, 5.1 Hz, 1H), 4.47-4.27 (m, 2H), 3.26 (dt, J=6.8, 4.1 Hz, 2H), 3.14 (s, 4H), 2.96 (ddd, J=18.1, 13.7, 5.4 Hz, 1H), 2.65 (t, J=7.8 Hz, 3H), 2.53 (s, 1H), 2.15-1.93 (m, 3H), 1.81 (s, 2H), 1.59 (s, 2H), 1.16 (s, 10H), 0.70 (h, J=6.4, 5.5 Hz, 1H), 0.44-0.33 (m, 2H), 0.04-−0.09 (m, 2H).

Compound 541A: LCMS: 511.4 (M+H). ¹H NMR (400 MHz, DMSO-d6): δ 11.03 (s, 1H), 7.47 (t, J=7.6 Hz, 1H), 7.33 (dd, J=14.7, 7.6 Hz, 2H), 5.17 (dd, J=13.2, 5.1 Hz, 1H), 4.55-4.29 (m, 2H), 3.28 (p, J=7.9, 6.8 Hz, 3H), 3.18 (d, J=1.2 Hz, 3H), 2.98 (ddd, J=18.2, 13.6, 5.5 Hz, 1H), 2.77 (s, 1H), 2.67 (d, J=18.5 Hz, 2H), 2.52 (s, 1H), 2.15-2.03 (m, 1H), 1.94 (t, J=11.5 Hz, 2H), 1.86-1.73 (m, 2H), 1.51 (t, J=7.2 Hz, 4H), 1.22 (s, 8H), 0.70 (h, J=7.0, 5.6 Hz, 1H), 0.47-0.35 (m, 2H), 0.05-−0.06 (m, 2H).

### Compound 542

### 3-{4-[(2-cyclopropylethyl)[(1r,4r)-4-[(2-methoxyethyl)amino]cyclohexyl]amino]-1-oxo-3H-isoindol-2-yl}piperidine-2,6-dione

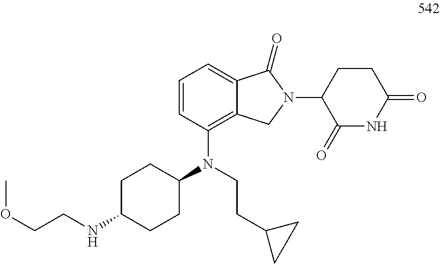 [SMILES: COCCN[C@H]1CC[C@H](N(CCC2CC2)c2cccc3c2CN(C2CCC(=O)NC2=O)C3=O)CC1]

The compound was prepared by the same procedure described in the preparation of Compound 540 above using the appropriate aldehyde or ketone.

LC-MS: (ES, m/z): [M+1]=483.25. ¹H-NMR (400 MHz, Methanol-d₄): δ 8.67 (s, 1H), 7.59-7.49 (m, 2H), 7.49-7.41 (m, 1H), 5.21 (dd, J=13.3, 5.1 Hz, 1H), 4.50 (s, 2H), 3.72-3.65 (m, 2H), 3.47 (s, 3H), 3.33 (td, J=7.1, 3.5 Hz, 3H), 3.28-3.05 (m, 4H), 2.98 (ddd, J=18.4, 13.4, 5.3 Hz, 1H), 2.85 (ddd, J=17.6, 4.7, 2.3 Hz, 1H), 2.61 (qd, J=13.1, 4.6 Hz, 1H), 2.24 (dtd, J=10.6, 5.3, 2.7 Hz, 3H), 2.08 (d, J=13.4 Hz, 2H), 1.76-1.65 (m, 2H), 1.58-1.45 (m, 2H), 1.36-1.18 (m, 2H), 0.78-0.66 (m, 1H), 0.44 (dt, J=8.0, 2.8 Hz, 2H).

### Compound 543

### 4-{[(1r,4r)-4-[(2-cyclopropylethyl)[2-(2,6-dioxopiperidin-3-yl)-1-oxo-3H-isoindol-4-yl]amino]cyclohexyl]amino}butanenitrile

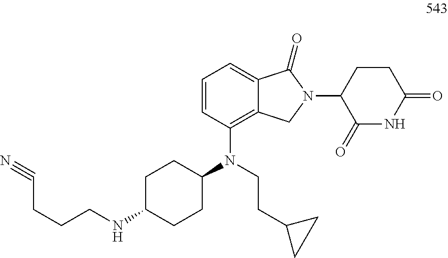 [SMILES: N#CCCCN[C@H]1CC[C@H](N(CCC2CC2)c2cccc3c2CN(C2CCC(=O)NC2=O)C3=O)CC1]

The compound was prepared by the same procedure described in the preparation of Compound 540 above using the appropriate aldehyde or ketone.

LC-MS: (ES, m/z): [M+1]=492.30. ¹H-NMR (400 MHz, Methanol-d₄): δ 7.61-7.52 (m, 2H), 7.48 (dd, J=7.0, 2.1 Hz, 1H), 5.20 (dd, J=13.3, 5.2 Hz, 1H), 4.51 (s, 2H), 3.36-3.30 (m, 1H), 3.29-3.11 (m, 4H), 2.97 (ddd, J=18.3, 13.5, 5.4 Hz, 1H), 2.85 (ddd, J=17.6, 4.7, 2.4 Hz, 1H), 2.71-2.54 (m, 3H), 2.25 (ddd, J=9.7, 5.4, 2.7 Hz, 3H), 2.08 (h, J=7.2, 6.6 Hz, 4H), 1.73 (q, J=13.5, 12.8 Hz, 2H), 1.53 (t, J=12.5 Hz, 2H), 1.35-1.18 (m, J=6.9 Hz, 2H), 0.78-0.65 (m, 1H), 0.48-0.40 (m, 2H).

### Compound 546

### 3-{4-[(2-cyclopropylethyl)[(1r,4r)-4-{[2-(azetidin-1-yl)-2-oxoethyl]amino}cyclohexyl]amino]-1-oxo-3H-isoindol-2-yl}piperidine-2,6 dione

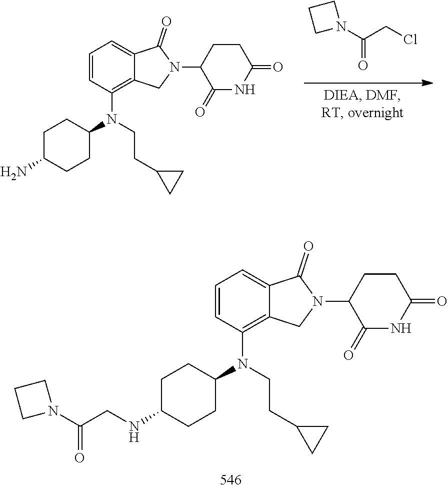 [SMILES: N[C@H]1CC[C@H](N(CCC2CC2)c2cccc3c2CN(C2CCC(=O)NC2=O)C3=O)CC1 ; O=C(CCl)N1CCC1 ; O=C1CCC(N2Cc3c(cccc3N(CCC3CC3)[C@H]3CC[C@H](NCC(=O)N4CCC4)CC3)C2=O)C(=O)N1]

A solution of 3-{4-[(2-cyclopropylethyl)[(1r,4r)-4-aminocyclohexyl]amino]-1-oxo-3H-isoindol-2-yl}piperidine-2,6-dione (200 mg, 0.47 mmol, 1 equiv), 1-(azetidin-1-yl)-2-chloroethanone (139 mg, 1.04 mmol, 2.2 equiv) and DIEA (182 mg, 1.32 mmol, 2.8 equiv) in DMF (4.0 mL) was stirred for overnight at room temperature under nitrogen atmosphere. The resulting mixture was diluted with water and extracted with EtOAc (3×200 mL). The combined organic layers were washed with water (2×100 mL), dried over anhydrous Na₂SO₄. After filtration, the filtrate was concentrated under reduced pressure. The crude product was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 7% B to 27% B in 8 min; Wave Length: 254/220 nm; RT1 (min): 7.13/8.13) to afford 3-{4-[(2-cyclopropylethyl)[(1r,4r)-4-{[2-(azetidin-1-yl)-2-oxoethyl]amino}cyclohexyl]amino]-1-oxo-3H-isoindol-2-yl}piperidine-2,6-dione (23 mg, 9%) as a white solid.

LC-MS: (ES, m/z): [M+1]=522.4 ¹H NMR (400 MHz, Methanol-d₄) δ 8.59 (s, 1H), 7.53 (d, J=4.4 Hz, 2H), 7.44 (p, J=3.8 Hz, 1H), 5.21 (dd, J=13.3, 5.1 Hz, 1H), 4.51 (s, 2H), 4.30 (t, J=7.7 Hz, 2H), 4.14 (t, J=7.9 Hz, 2H), 3.78 (s, 2H), 3.36 (s, 1H), 3.33 (t, J=7.3 Hz, 2H), 3.20 (tt, J=12.3, 3.5 Hz, 1H), 3.11-2.99 (m, 1H), 3.00-2.90 (m, 1H), 2.85 (ddd, J=17.6, 4.8, 2.4 Hz, 1H), 2.62 (qd, J=13.2, 4.7 Hz, 1H), 2.43 (ddd, J=18.2, 9.1, 6.0 Hz, 3H), 2.30-2.13 (m, 3H), 2.06 (d, J=11.3 Hz, 2H), 1.69 (q, J=12.9 Hz, 2H), 1.52 (q, J=13.6, 13.0 Hz, 2H), 1.27 (qd, J=6.9, 3.9 Hz, 2H), 0.72 (ddt, J=12.5, 7.6, 3.7 Hz, 1H), 0.49-0.36 (m, 2H), 0.04-0.01 (m, 2H).

### Compound 547

### 3-{4-[(2-cyclopropylethyl)[(1r,4r)-4-[(4,4,4-trifluorobutan-2-yl)amino]cyclohexyl]amino]-1-oxo-3H-isoindol-2-yl}piperidine-2,6-dione

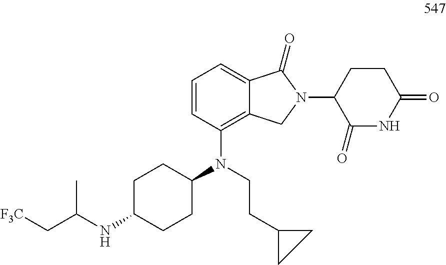 [SMILES: CC(CC(F)(F)F)N[C@H]1CC[C@H](N(CCC2CC2)c2cccc3c2CN(C2CCC(=O)NC2=O)C3=O)CC1]

The compound was prepared by the same procedure described in the preparation of Compound 540 above using the appropriate aldehyde or ketone.

LC-MS: (ES, m/z): [M+1]=535.35; ¹H NMR (400 MHz, DMSO-d₆) δ 10.96 (s, 1H), 7.40 (t, J=7.7 Hz, 1H), 7.24 (dd, J=19.5, 7.7 Hz, 2H), 5.09 (dd, J=13.1, 5.1 Hz, 1H), 4.38-4.19 (m, 2H), 3.23-3.17 (m, 2H), 3.12-3.03 (m, 1H), 3.02-2.84 (m, 2H), 2.65-2.52 (m, 2H), 2.48-2.38 (m, 2H), 2.37-2.28 (m, 1H), 2.13 (dqd, J=14.8, 11.8, 7.2 Hz, 1H), 2.00 (ddd, J=10.4, 6.1, 3.8 Hz, 1H), 1.83 (t, J=13.5 Hz, 2H), 1.72 (d, J=12.2 Hz, 2H), 1.62-1.45 (m, 2H), 1.14 (q, J=7.3 Hz, 2H), 1.06 (d, J=6.3 Hz, 5H), 0.64 (ddt, J=12.6, 7.9, 3.8 Hz, 1H), 0.33 (dt, J=8.1, 2.8 Hz, 2H), −0.07 (d, J=4.8 Hz, 2H).

### Compound 548 and 548A

### 3-(4-((2-cyclopropylethyl)((1r,4r)-4-(((R)-1,1,1-trifluoropropan-2-yl)amino)cyclohexyl)amino)-1-oxoisoindolin-2-yl)piperidine-2,6-dione and 3-(4-((2-cyclopropylethyl)((1s,4s)-4-(((R)-1,1,1-trifluoropropan-2-yl)amino)cyclohexyl)amino)-1-oxoisoindolin-2-yl)piperidine-2,6-dione

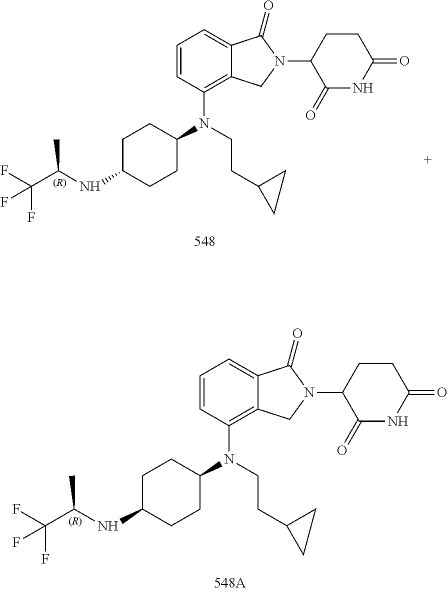

The compounds were prepared by the general procedure described in the preparation of Compounds 515 and 515A using the appropriate amine.

Compound 548: LCMS: 521.3 (M+H). ¹H NMR (400 MHz, DMSO-d6): δ 11.02 (s, 1H), 7.46 (t, J=7.7 Hz, 1H), 7.30 (dd, J=21.0, 7.6 Hz, 2H), 5.15 (dd, J=13.1, 5.1 Hz, 1H), 4.45-4.27 (m, 2H), 3.39-3.31 (m, 2H), 3.30-3.21 (m, 2H), 3.14 (tt, J=11.8, 3.6 Hz, 1H), 2.96 (ddd, J=17.8, 13.5, 5.4 Hz, 1H), 2.72-2.59 (m, 2H), 2.53-2.45 (m, 1H), 2.14-1.87 (m, 3H), 1.79 (d, J=14.5 Hz, 2H), 1.68-1.49 (m, 2H), 1.29-1.03 (m, 7H), 0.69 (qq, J=7.2, 4.9, 3.7 Hz, 1H), 0.39 (dt, J=8.4, 2.8 Hz, 2H), −0.01 (q, J=4.8 Hz, 2H).

Compound 548A: LCMS: 521.3 (M+H). ¹H NMR (400 MHz, DMSO-d6): δ 11.02 (s, 1H), 7.46 (t, J=7.7 Hz, 1H), 7.31 (dd, J=15.2, 7.6 Hz, 2H), 5.15 (dd, J=13.1, 5.1 Hz, 1H), 4.53-4.26 (m, 2H), 3.35-3.11 (m, 5H), 3.05-2.80 (m, 2H), 2.70-2.60 (m, 2H), 2.13-1.84 (m, 3H), 1.75 (t, J=16.4 Hz, 2H), 1.59-1.38 (m, 4H), 1.24 (t, J=6.0 Hz, 5H), 0.77-0.62 (m, 1H), 0.46-0.34 (m, 2H), 0.04-−0.06 (m, 2H).

### Compound 550

### 3-(4-{[(3S,3aR,6S,6aR)-6-amino-hexahydrofuro[3,2-b]furan-3-yl](2-cyclopropylethyl)amino}-1-oxo-3H-isoindol-2-yl)piperidine-2,6-dione

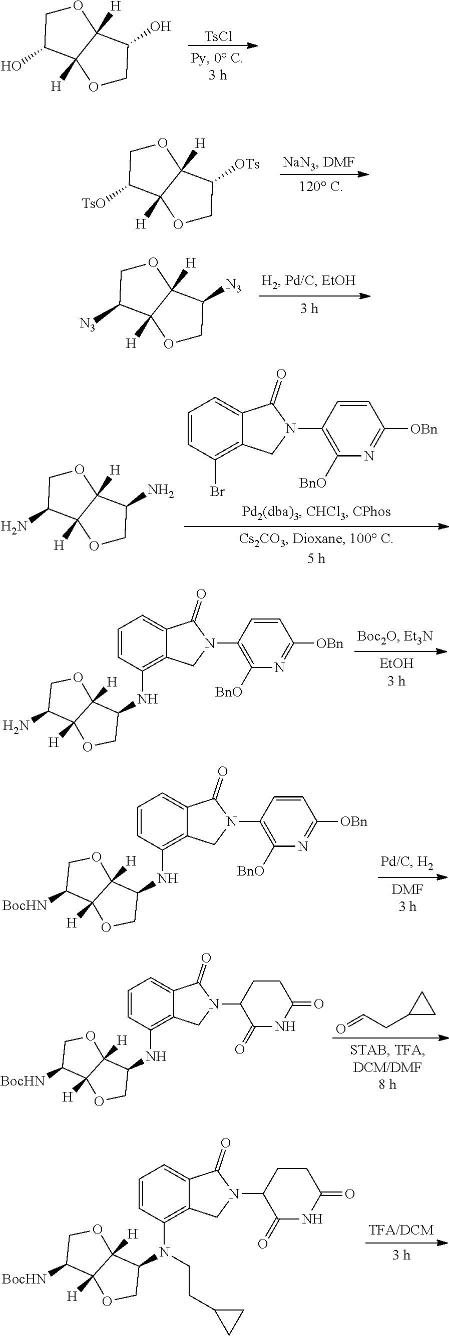

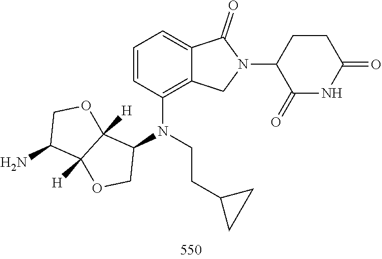 [SMILES: N[C@H]1CO[C@H]2[C@@H]1OC[C@@H]2N(CCC1CC1)c1cccc2c1CN(C1CCC(=O)NC1=O)C2=O]

### Step-1: (3R,3aS,6R,6aS)-6-[(4-methylbenzenesulfonyl)oxy]-hexahydrofuro[3,2-b]furan-3-yl 4-methylbenzenesulfonate

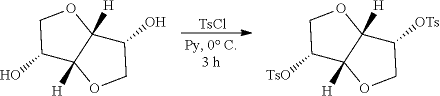 [SMILES: O[C@@H]1CO[C@H]2[C@@H]1OC[C@H]2O ; "TsCl" ; Cc1ccc(S(=O)(=O)O[C@@H]2CO[C@H]3[C@@H]2OC[C@H]3OS(=O)(=O)c2ccc(C)cc2)cc1]

Into a 250 mL round-bottom flask were added mannitol, 1,4:3,6-dianhydro (5 g, 34.2 mmol, 1 equiv) and Pyridine (30 mL) at room temperature. The resulting mixture was stirred for 0.3 h at room temperature. P-toluenesulfonyl chloride (19.5 g, 102 mmol, 3 equiv) was added at 0° C. The resulting mixture was stirred for 3 h at room temperature. The residue was diluted with H₂O (300 mL). The resulting mixture was extracted with EA (3×100 mL). The combined organic layers were washed with H₂O (3×50 mL), dried over anhydrous Na₂SO₄. After filtration, the filtrate was concentrated under reduced pressure. This resulted in (3R,3aS,6R,6aS)-6-[(4-methylbenzenesulfonyl)oxy]-hexahydrofuro[3,2-b]furan-3-yl 4-methylbenzenesulfonate (15 g, crude) as a yellow oil.

### Step-2: (3S,3aR,6S,6aR)-3,6-diazido-hexahydrofuro[3,2-b]furan

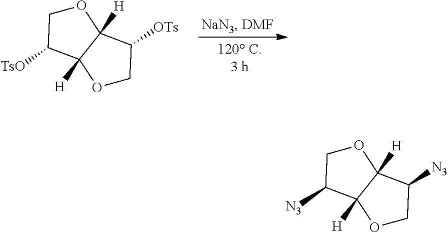

Into a 40 mL sealed tube were added (3R,3aS,6R,6aS)-6-[(4-methylbenzenesulfonyl)oxy]-hexahydrofuro[3,2-b]furan-3-yl 4-methylbenzenesulfonate (5 g, 11.0 mmol, 1 equiv), NaN₃ (2.15 g, 33.0 mmol, 3 equiv) and DMF (20 mL) at room temperature. The resulting mixture was stirred for 3 h at 120° C. The reaction was cooled down to RT and diluted with water (50 mL). The resulting mixture was extracted with EA (3×100 mL). The combined organic layers were washed with H₂O (3×50 mL), dried over anhydrous Na₂SO₄. After filtration, the filtrate was concentrated under reduced pressure. This resulted in (3S,3aR,6S,6aR)-3,6-diazido-hexahydrofuro[3,2-b]furan (1.9 g, crude) as a yellow liquid.

### Step-3: in (3S,3aR,6S,6aR)-hexahydrofuro[3,2-b]furan-3,6-diamine

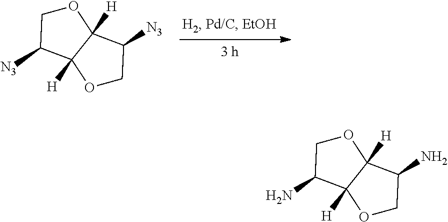 [SMILES: [N-]=[N+]=N[C@H]1CO[C@H]2[C@@H]1OC[C@@H]2N=[N+]=[N-] ; N[C@H]1CO[C@H]2[C@@H]1OC[C@@H]2N]

Into a 100 mL round-bottom flask were added (3S,3aR,6S,6aR)-3,6-diazido-hexahydrofuro[3,2-b]furan 3 (2 g, 10.2 mmol, 1 equiv), Palladium 10% on Carbon (wetted with ca. 55% Water, 2.17 g, 20.4 mmol, 2 equiv) and EtOH (10 mL) at room temperature. The resulting mixture was stirred for 3 h at room temperature under hydrogen atmosphere. The resulting mixture was filtered, the filter cake was washed with THF (3×5 mL). The filtrate was concentrated under reduced pressure. The residue was purified by reversed-phase flash chromatography with the following conditions: column, C18 330 silica gel; mobile phase, ACN in Water (0.1% FA); detector, UV 254 nm. This resulted in (3S,3aR,6S,6aR)-hexahydrofuro[3,2-b]furan-3,6-diamine (1.3 g, 88%) as a white solid.

### Step-4: 4-{[(3S,3aR,6S,6aR)-6-amino-hexahydrofuro[3,2-b]furan-3-yl]amino}-2-[2,6-bis(benzyloxy)pyridin-3-yl]-3H-isoindol-1-one

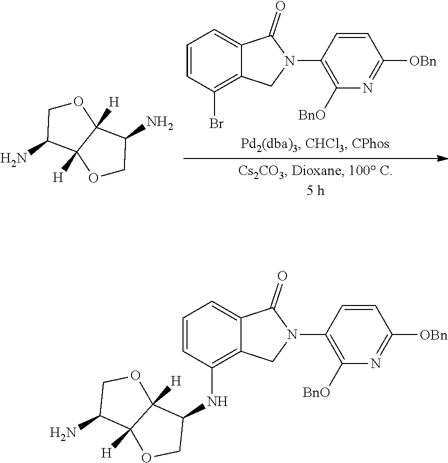 [SMILES: N[C@H]1CO[C@H]2[C@@H]1OC[C@@H]2N ; O=C1c2cccc(Br)c2CN1c1ccc(OCc2ccccc2)nc1OCc1ccccc1 ; N[C@H]1CO[C@H]2[C@@H]1OC[C@@H]2Nc1cccc2c1CN(c1ccc(OCc3ccccc3)nc1OCc1ccccc1)C2=O]

Into a 100 mL round-bottom flask were added (3S,3aR,6S,6aR)-hexahydrofuro[3,2-b]furan-3,6-diamine (1 g, 6.93 mmol, 3 equiv), 4-amino-2-[2,6-bis(benzyloxy)pyridin-3-yl]-3H-isoindol-1-one (1.01 g, 2.3 mmol, 1 equiv), Pd₂(dba)₃·CHCl₃ (478 mg, 0.46 mmol, 0.2 equiv), CPhos (201 mg, 0.46 mmol, 0.2 equiv), Cs₂CO₃ (2.26 g, 6.93 mmol, 3 equiv) and dioxane (50 mL) at 100° C. under nitrogen atmosphere. The resulting mixture was stirred for 5 h at 100° C. The mixture was allowed to cool down to room temperature. The residue was diluted with H₂O (300 mL). The resulting mixture was extracted with EA (3×100 mL). The combined organic layers were washed with H₂O (3×50 mL), dried over anhydrous Na₂SO₄. After filtration, the filtrate was concentrated under reduced pressure. The residue was purified by reversed-phase flash chromatography with the following conditions: column, Cis 120 silica gel; mobile phase, ACN in Water (0.1% FA); detector, UV 254 nm. This resulted in 4-{[(3S,3aR,6S,6aR)-6-amino-hexahydrofuro[3,2-b]furan-3-yl]amino}-2-[2,6-bis(benzyloxy)pyridin-3-yl]-3H-isoindol-1-one (1.0 g, 76%) as a white solid.

### Step-5: tert-butyl N-[(3S,3aR,6S,6aR)-6-({2-[2,6-bis(benzyloxy)pyridin-3-yl]-1-oxo-3H-isoindol-4-yl}amino)-hexahydrofuro[3,2-b]furan-3-yl]carbamate

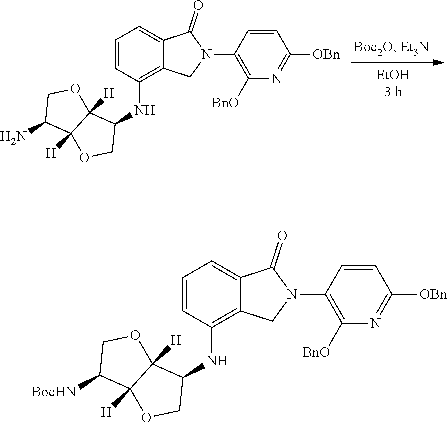 [SMILES: N[C@H]1CO[C@H]2[C@@H]1OC[C@@H]2Nc1cccc2c1CN(c1ccc(OCc3ccccc3)nc1OCc1ccccc1)C2=O ; CC(C)(C)OC(=O)N[C@H]1CO[C@H]2[C@@H]1OC[C@@H]2Nc1cccc2c1CN(c1ccc(OCc3ccccc3)nc1OCc1ccccc1)C2=O]

Into a 50 mL round-bottom flask were added 4-{[(3S,3aR,6S,6aR)-6-amino-hexahydrofuro[3,2-b]furan-3-yl]amino}-2-[2,6-bis(benzyloxy)pyridin-3-yl]-3H-isoindol-1-one (430 mg, 0.762 mmol, 1 equiv), di-tert-butyl dicarbonate (332 mg, 1.52 mmol, 2 equiv), Et₃N (231 mg, 2.28 mmol, 3 equiv) and ethyl alcohol (20 mL) at room temperature. The resulting mixture was stirred for 3 h at room temperature. The resulting mixture was concentrated under vacuum. The residue was purified by reversed-phase flash chromatography with the following conditions: column, Cis 120 silica gel; mobile phase, ACN in Water (0.1% FA); detector, UV 254 nm. This resulted in tert-butyl N-[(3S,3aR,6S,6aR)-6-({2-[2,6-bis(benzyloxy)pyridin-3-yl]-1-oxo-3H-isoindol-4-yl}amino)-hexahydrofuro[3,2-b]furan-3-yl]carbamate (420 mg, 83%) as a white solid.

### Step-6: tert-butyl N-[(3S,3aR,6S,6aR)-6-{[2-(2,6-dioxopiperidin-3-yl)-1-oxo-3H-isoindol-4-yl]amino}-hexahydrofuro[3,2-b]furan-3-yl]carbamate

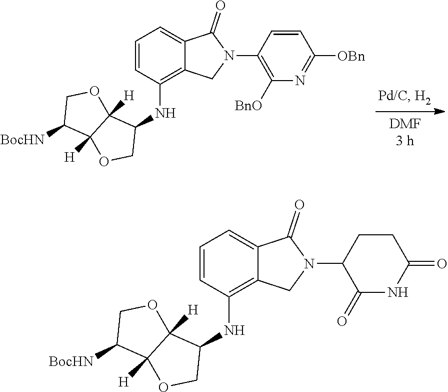 [SMILES: CC(C)(C)OC(=O)N[C@H]1CO[C@H]2[C@@H]1OC[C@@H]2Nc1cccc2c1CN(c1ccc(OCc3ccccc3)nc1OCc1ccccc1)C2=O ; CC(C)(C)OC(=O)N[C@H]1CO[C@H]2[C@@H]1OC[C@@H]2Nc1cccc2c1CN(C1CCC(=O)NC1=O)C2=O]

Into a 25 mL round-bottom flask were added tert-butyl N-[(3S,3aR,6S,6aR)-6-({2-[2,6-bis(benzyloxy)pyridin-3-yl]-1-oxo-3H-isoindol-4-yl}amino)-hexahydrofuro[3,2-b]furan-3-yl]carbamate (420 mg, 0.63 mmol, 1 equiv), Palladium 10% on Carbon (wetted with ca. 55% Water, 336 mg, 3.16 mmol, 5 equiv) and dimethylformamide (10 mL) at room temperature. The resulting mixture was stirred for 3 h at room temperature under hydrogen atmosphere. The resulting mixture was filtered, the filter cake was washed with THF (3×5 mL). The residue was diluted with H₂O (30 mL). The resulting mixture was extracted with EA (3×10 mL). The combined organic layers were washed with H₂O (3×50 mL), dried over anhydrous Na₂SO₄. After filtration, the filtrate was concentrated under reduced pressure. The residue was purified by reversed-phase flash chromatography with the following conditions: column, Cis 120 silica gel; mobile phase, ACN in Water (0.1% FA); detector, UV 254 nm. This resulted in tert-butyl N-[(3S,3aR,6S,6aR)-6-{[2-(2,6-dioxopiperidin-3-yl)-1-oxo-3H-isoindol-4-yl]amino}-hexahydrofuro[3,2-b]furan-3-yl]carbamate (280 mg, 91%) as a white solid.

### Step-7: tert-butyl N-[(3S,3aR,6S,6aR)-6-[(2-cyclopropylethyl)[2-(2,6-dioxopiperidin-3-yl)-1-oxo-3H-isoindol-4-yl]amino]-hexahydrofuro[3,2-b]furan-3-yl]carbamate

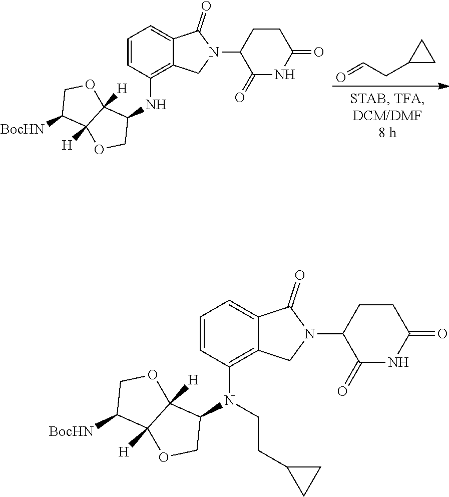

Into a 25 mL round-bottom flask were added tert-butyl N-[(3S,3aR,6S,6aR)-6-{[2-(2,6-dioxopiperidin-3-yl)-1-oxo-3H-isoindol-4-yl]amino}-hexahydrofuro[3,2-b]furan-3-yl]carbamate (130 mg, 0.267 mmol, 1 equiv), 2-cyclopropylacetaldehyde (224 mg, 2.67 mmol, 10 equiv), TFA (91 mg, 0.80 mmol, 3 equiv) and DMF (0.6 mL), DCM (2.5 mL) at room temperature. The resulting mixture was stirred for 8 h at room temperature. STAB (283 mg, 1.3 mmol, 5 equiv) was added at 0° C. The resulting mixture was stirred for additional 3 h at room temperature. The resulting mixture was concentrated under vacuum. The residue was diluted with H₂O (300 mL). The resulting mixture was extracted with EA (3×100 mL). The combined organic layers were washed with H₂O (3×50 mL), dried over anhydrous Na₂SO₄. After filtration, the filtrate was concentrated under reduced pressure. The residue was purified by reversed-phase flash chromatography with the following conditions: column, C18 120 silica gel; mobile phase, ACN in Water (0.1% FA); detector, UV 254 nm. This resulted in tert-butyl N-[(3S,3aR,6S,6aR)-6-[(2-cyclopropylethyl)[2-(2,6-dioxopiperidin-3-yl)-1-oxo-3H-isoindol-4-yl]amino]-hexahydrofuro[3,2-b]furan-3-yl]carbamate (110 mg, 74%) as a white solid.

### Step-8: 3-(4-{[(3S,3aR,6S,6aR)-6-amino-hexahydrofuro[3,2-b]furan-3-yl](2-cyclopropylethyl)amino}-1-oxo-3H-isoindol-2-yl)piperidine-2,6-dione

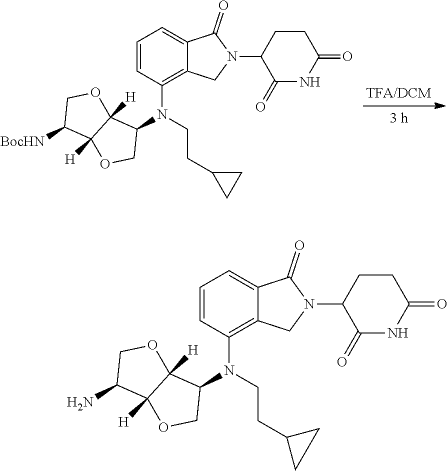 [SMILES: CC(C)(C)OC(=O)N[C@H]1CO[C@H]2[C@@H]1OC[C@@H]2N(CCC1CC1)c1cccc2c1CN(C1CCC(=O)NC1=O)C2=O ; N[C@H]1CO[C@H]2[C@@H]1OC[C@@H]2N(CCC1CC1)c1cccc2c1CN(C1CCC(=O)NC1=O)C2=O]

Into a 25 mL round-bottom flask were added tert-butyl N-[(3S,3aR,6S,6aR)-6-[(2-cyclopropylethyl)[2-(2,6-dioxopiperidin-3-yl)-1-oxo-3H-isoindol-4-yl]amino]-hexahydrofuro[3,2-b]furan-3-yl]carbamate (80 mg, 0.14 mmol, 1 equiv) and DCM (5 mL) at room temperature. TFA (0.5 mL, 6.7 mmol, 46 equiv) at was added dropwise at 0° C. The resulting mixture was stirred for 3 h at room temperature. The resulting mixture was concentrated under vacuum. The residue was purified by Prep-HPLC with the following conditions: Column: XBridge Prep Phenyl OBD Column 19*250 mm, 5 m; Mobile Phase A: Water (0.05% TFA), Mobile Phase B: ACN; Flow rate: 60 mL/min mL/min; Gradient: 57% B to 66% B in 8 min; Wave Length: 254 nm/220 nm; RT1 (min): 8.23. This resulted in 3-(4-{[(3S,3aR,6S,6aR)-6-amino-hexahydrofuro[3,2-b]furan-3-yl](2-cyclopropylethyl)amino}-1-oxo-3H-isoindol-2-yl)piperidine-2,6-dione (50 mg, 75%) as a white solid.

LCMS: 455.2 (M+H). ¹H NMR (400 MHz, DMSO-d6) δ 11.05 (s, 1H), 8.25 (d, J=5.1 Hz, 3H), 7.55 (dd, J=8.7, 6.3 Hz, 1H), 7.49 (dd, J=6.3, 3.2 Hz, 2H), 5.18 (dt, J=12.5, 5.8 Hz, 1H), 4.70 (dd, J=4.9, 2.0 Hz, 1H), 4.66-4.58 (m, 1H), 4.56-4.26 (m, 2H), 4.09 (dd, J=9.4, 6.3 Hz, 1H), 4.05-3.97 (m, 2H), 3.85 (dd, J=10.6, 2.1 Hz, 1H), 3.79 (d, J=4.9 Hz, 1H), 3.79-3.70 (m, 1H), 3.26 (tt, J=16.6, 8.5 Hz, 2H), 2.98 (ddd, J=17.9, 10.1, 5.0 Hz, 1H), 2.66 (d, J=17.8 Hz, 1H), 2.50 (td, J=12.7, 4.6 Hz, 1H), 2.13-2.03 (m, 1H), 1.22 (tdd, J=21.6, 10.8, 4.8 Hz, 2H), 0.66 (td, J=8.1, 4.7 Hz, 1H), 0.40 (dd, J=8.7, 4.5 Hz, 2H).

### Compounds 554 and 554A

### 3-(4-(((1r,4r)-4-((3,3-difluorocyclobutyl)amino)cyclohexyl)(2-(1-(trifluoromethyl)cyclopropyl)ethyl)amino)-1-oxoisoindolin-2-yl)piperidine-2,6-dione formate and 3-(4-(((1s,4s)-4-((3,3-difluorocyclobutyl)amino)cyclohexyl)(2-(1-(trifluoromethyl)cyclopropyl)ethyl)amino)-1-oxoisoindolin-2-yl)piperidine-2,6-dione

### Step-1: [1-(trifluoromethyl)cyclopropyl]methanol

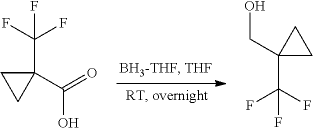 [SMILES: O=C(O)C1(C(F)(F)F)CC1 ; OCC1(C(F)(F)F)CC1]

A solution of 1-(trifluoromethyl)cyclopropane-1-carboxylic acid (10.0 g, 64.8 mmol, 1 equiv) in THF (250 mL) was added BH₃-THF (162 mL, 162 mmol, 2.5 equiv) dropwise at −10° C. under nitrogen atmosphere. The resulting mixture was stirred for overnight at room temperature under nitrogen atmosphere. The reaction was quenched with K₂CO₃ (aq.) at 0° C. The resulting mixture was extracted with EtOEt (3×250 mL). The combined organic layers were washed with brine (3×150 mL), dried over anhydrous Na₂SO₄. After filtration, the filtrate was concentrated under reduced pressure. The crude product, [1-(trifluoromethyl)cyclopropyl]methanol, was used in the next step directly without further purification.

### Step-2: [1-(trifluoromethyl)cyclopropyl]methyl 4-methylbenzenesulfonate

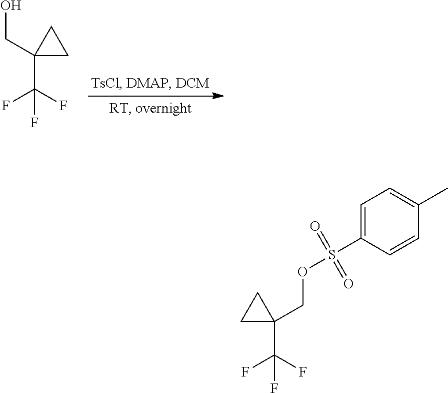 [SMILES: OCC1(C(F)(F)F)CC1 ; Cc1ccc(S(=O)(=O)OCC2(C(F)(F)F)CC2)cc1]

To a stirred solution of [1-(trifluoromethyl)cyclopropyl]methanol (10.0 g, 71.3 mmol, 1 equiv) and DMAP (4.4 g, 35.6 mmol, 0.5 equiv) in DCM (390 mL) were added TEA (28.9 g, 285 mmol, 4 equiv) and P-toluenesulfonyl chloride (34 g, 178 mmol, 2.5 equiv) in portions at 0° C. under nitrogen atmosphere. The resulting mixture was stirred for overnight at room temperature under nitrogen atmosphere. The resulting mixture was diluted with water (500 mL). The resulting mixture was extracted with CH₂Cl₂ (3×100 mL). The combined organic layers were washed with brine (3×100 mL), dried over anhydrous Na₂SO₄. After filtration, the filtrate was concentrated under reduced pressure. The residue was purified by silica gel column chromatography, eluted with PE/EA (5:1) to afford [1-(trifluoromethyl)cyclopropyl]methyl 4-methylbenzenesulfonate (11 g, 52%) as a yellow liquid.

### Step-3: 2-[1-(trifluoromethyl)cyclopropyl]acetonitrile

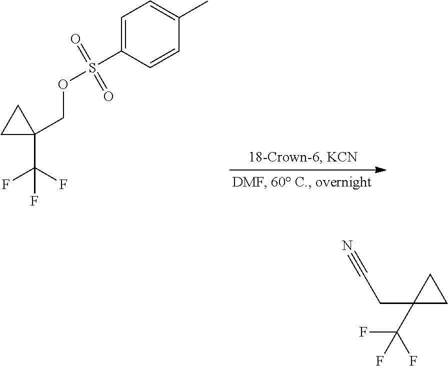 [SMILES: Cc1ccc(S(=O)(=O)OCC2(C(F)(F)F)CC2)cc1 ; N#CCC1(C(F)(F)F)CC1]

To a stirred solution of [1-(trifluoromethyl)cyclopropyl]methyl 4-methylbenzenesulfonate (11.0 g, 37.4 mmol, 1 equiv) and 18-crown-6 (14.8 g, 56 mmol, 1.5 equiv) in DMF (110 mL) was added KCN (3.7 g, 56.1 mmol, 1.5 equiv) at room temperature under nitrogen atmosphere. The resulting mixture was stirred for overnight at 60° C. under nitrogen atmosphere. The resulting mixture was diluted with water (500 mL). The resulting mixture was extracted with EtOAc (3×200 mL). The combined organic layers were washed with brine (3×200 mL), dried over anhydrous Na₂SO₄. After filtration, the filtrate was concentrated under reduced pressure. The crude product, 2-[1-(trifluoromethyl)cyclopropyl]acetonitrile, was used in the next step directly without further purification.

### Step-4: [1-(trifluoromethyl)cyclopropyl]acetic acid

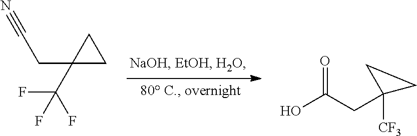 [SMILES: N#CCC1(C(F)(F)F)CC1 ; O=C(O)CC1(C(F)(F)F)CC1]

A solution of 2-[1-(trifluoromethyl)cyclopropyl]acetonitrile (5.2 g, 34.8 mmol, 1 equiv) in EtOH (157 mL) and H₂O (52 mL) was added NaOH (27.9 g, 697 mmol, 20 equiv) at room temperature under nitrogen atmosphere. The resulting mixture was stirred for overnight at 80° C. under nitrogen atmosphere. The resulting mixture was diluted with water (500 mL). The resulting mixture was acidified to pH 2 with 1M HCl (aq.). The resulting mixture was extracted with EtOAc (3×500 mL). The combined organic layers were washed with brine (3×100 mL), dried over anhydrous Na₂SO₄. After filtration, the filtrate was concentrated under reduced pressure. The crude product, [1-(trifluoromethyl)cyclopropyl]acetic acid, was used in the next step directly without further purification.

### Step-5: 2-[1-(trifluoromethyl)cyclopropyl]ethanol

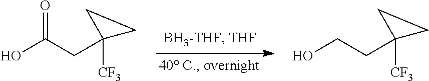 [SMILES: O=C(O)CC1(C(F)(F)F)CC1 ; OCCC1(C(F)(F)F)CC1]

A solution of [1-(trifluoromethyl)cyclopropyl]acetic acid (5.2 g, 30.9 mmol, 1 equiv) in THF (78 mL) was added BH₃-THF (61.8 mL, 61.8 mmol, 2.0 equiv) dropwise at 0° C. under nitrogen atmosphere. The resulting mixture was stirred for overnight at 40° C. under nitrogen atmosphere. The reaction was quenched with sat. NH₄Cl (aq.) at 0° C. The resulting mixture was extracted with CH₂Cl₂ (3×200 mL). The combined organic layers were washed with brine (3×100 mL), dried over anhydrous Na₂SO₄. After filtration, the filtrate was concentrated under reduced pressure. The crude product, 2-[1-(trifluoromethyl)cyclopropyl]ethanol, was used in the next step directly without further purification.

### Step-6: 3-(4-{1,4-dioxaspiro[4.5]decan-8-ylamino}-1-oxo-3H-isoindol-2-yl)piperidine-2,6-dione

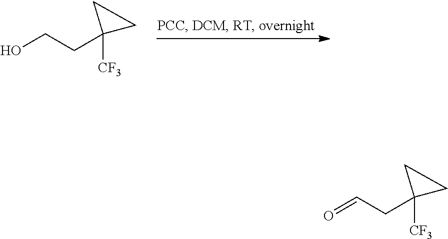 [SMILES: OCCC1(C(F)(F)F)CC1 ; O=CCC1(C(F)(F)F)CC1]

A solution of 2-[1-(trifluoromethyl)cyclopropyl]ethanol (2.0 g, 12.9 mmol, 1 equiv) and PCC (4.2 g, 19.4 mmol, 1.5 equiv) in DCM (40 mL) was stirred for overnight at room temperature under nitrogen atmosphere. The resulting mixture was filtered, the filter cake was washed with CH₂Cl₂ (3×10 mL). The filtrate was concentrated under reduced pressure. The crude product, 3-(4-{1,4-dioxaspiro[4.5]decan-8-ylamino}-1-oxo-3H-isoindol-2-yl)piperidine-2,6-dione, was used in the next step directly without further purification.

### Step-7: 3-(4-{1,4-dioxaspiro[4.5]decan-8-yl({2-[1-(trifluoromethyl)cyclopropyl]ethyl})amino}-1-oxo-3H-isoindol-2-yl)piperidine-2,6-dione

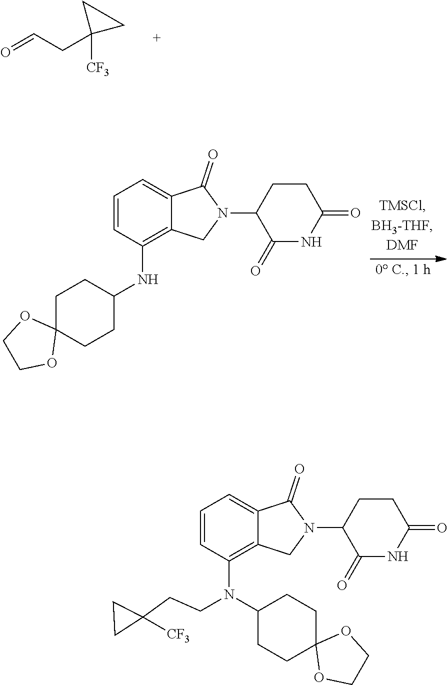 [SMILES: O=CCC1(C(F)(F)F)CC1 ; O=C1CCC(N2Cc3c(NC4CCC5(CC4)OCCO5)cccc3C2=O)C(=O)N1 ; O=C1CCC(N2Cc3c(cccc3N(CCC3(C(F)(F)F)CC3)C3CCC4(CC3)OCCO4)C2=O)C(=O)N1]

To a stirred solution of 3-(4-{1,4-dioxaspiro[4.5]decan-8-ylamino}-1-oxo-3H-isoindol-2-yl)piperidine-2,6-dione (400 mg, 1.0 mmol, 1 equiv) and 2-[1-(trifluoromethyl)cyclopropyl]acetaldehyde (1.5 g, 10.0 mmol, 10 equiv) in DMF (8.00 mL) was added TMSCl (1.28 mL, 10.0 mmol, 10 equiv) dropwise at 0° C. under nitrogen atmosphere. The resulting mixture was stirred for 1 h at 0° C. under nitrogen atmosphere. To the above mixture was added BH₃-THF (3.00 mL, 3.0 mmol, 3 equiv) dropwise over 2 min at 0° C. The resulting mixture was stirred for additional 1 h at 0° C. The reaction was quenched by the addition of sat. NH₄Cl (aq.) (100 mL) at 0° C. The resulting mixture was extracted with EtOAc (3×200 mL). The combined organic layers were washed with brine (3×200 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 0% to 100% gradient in 10 min; detector, UV 254 nm to afford 3-(4-{1,4-dioxaspiro[4.5]decan-8-yl({2-[1-(trifluoromethyl)cyclopropyl]ethyl})amino}-1-oxo-3H-isoindol-2-yl)piperidine-2,6-dione (300 mg, 56%) as a white solid.

### Step-8: 3-{1-oxo-4-[(4-oxocyclohexyl)({2-[1-(trifluoromethyl)cyclopropyl]ethyl})amino]-3H-isoindol-2-yl}piperidine-2,6-dione

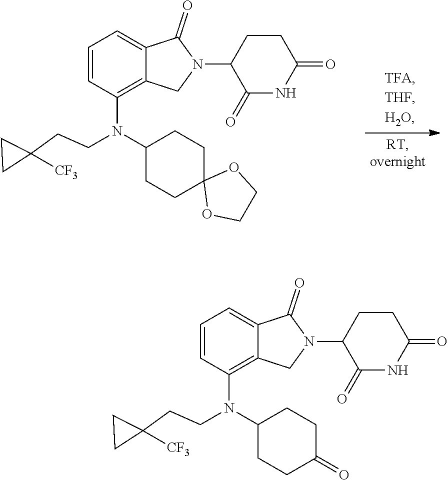 [SMILES: O=C1CCC(N2Cc3c(cccc3N(CCC3(C(F)(F)F)CC3)C3CCC4(CC3)OCCO4)C2=O)C(=O)N1 ; O=C1CCC(N(CCC2(C(F)(F)F)CC2)c2cccc3c2CN(C2CCC(=O)NC2=O)C3=O)CC1]

To a stirred solution of 3-(4-{1,4-dioxaspiro[4.5]decan-8-yl({2-[1-(trifluoromethyl)cyclopropyl]ethyl})amino}-1-oxo-3H-isoindol-2-yl)piperidine-2,6-dione (300 mg, 0.56 mmol, 1 equiv) in THF (3.0 mL) and H₂O (3.0 mL) was added TFA (3.00 mL) dropwise at room temperature under nitrogen atmosphere. The resulting mixture was stirred for overnight at room temperature under nitrogen atmosphere. The resulting mixture was diluted with water (100 mL). The resulting mixture was extracted with EtOAc (3×200 mL). The combined organic layers were washed with brine (3×200 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 0% to 100% gradient in 10 min; detector, UV 254 nm to afford 3-{1-oxo-4-[(4-oxocyclohexyl)({2-[1-(trifluoromethyl)cyclopropyl]ethyl})amino]-3H-isoindol-2-yl}piperidine-2,6-dione (160 mg, 58%) as a white solid.

### Step-9: 3-(4-(((1r,4r)-4-((3,3-difluorocyclobutyl)amino)cyclohexyl)(2-(1-(trifluoromethyl)cyclopropyl)ethyl)amino)-1-oxoisoindolin-2-yl)piperidine-2,6-dione formate and 3-(4-(((1s,4s)-4-((3,3-difluorocyclobutyl)amino)cyclohexyl)(2-(1-(trifluoromethyl)cyclopropyl)ethyl)amino)-1-oxoisoindolin-2-yl)piperidine-2,6-dione

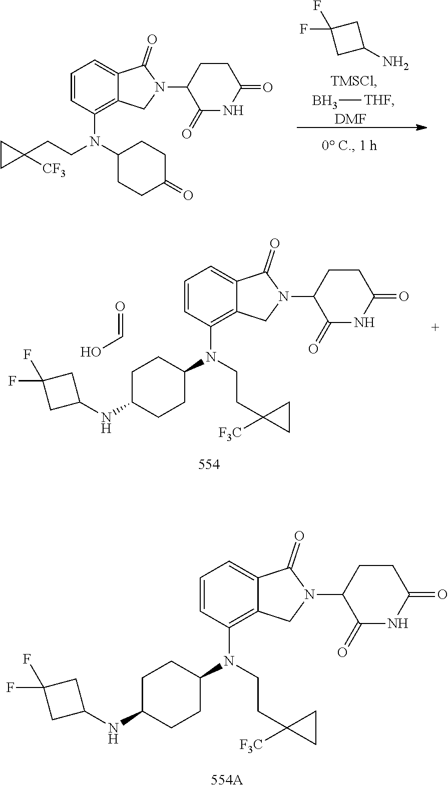

To a stirred solution of 3-{1-oxo-4-[(4-oxocyclohexyl)({2-[1-(trifluoromethyl)cyclopropyl]ethyl})amino]-3H-isoindol-2-yl}piperidine-2,6-dione (190 mg, 0.38 mmol, 1 equiv) and 3,3-difluorocyclobutan-1-amine (207 mg, 1.93 mmol, 5 equiv) in DMF (4 mL) was added TMSCl (0.49 mL, 3.87 mmol, 10 equiv) dropwise at 0° C. under nitrogen atmosphere. The resulting mixture was stirred for 1 h at 0° C. under nitrogen atmosphere. To the above mixture was added BH₃-THF (1.16 mL, 1.16 mmol, 3 equiv) dropwise over 1 min at 0° C. The resulting mixture was stirred for additional 1 h at 0° C. The reaction was quenched by the addition of sat. NH₄Cl (aq.) (100 mL) at 0° C. The resulting mixture was extracted with EtOAc (3×200 mL). The combined organic layers were washed with brine (3×200 mL), dried over anhydrous Na₂SO₄. After filtration, the filtrate was concentrated under reduced pressure. The crude product (200 mg) was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 10% B to 30% B in 10 min; Wave Length: 254/220 nm; RT1 (min): 10.03/12.0) to afford 3-(4-(((1r,4r)-4-((3,3-difluorocyclobutyl)amino)cyclohexyl)(2-(1-(trifluoromethyl)cyclopropyl)ethyl)amino)-1-oxoisoindolin-2-yl)piperidine-2,6-dione formate (148 mg, 61%) as an off-white solid and 3-(4-(((1s,4s)-4-((3,3-difluorocyclobutyl)amino)cyclohexyl)(2-(1-(trifluoromethyl)cyclopropyl)ethyl)amino)-1-oxoisoindolin-2-yl)piperidine-2,6-dione (8.2 mg, 3.6%) as an off-white solid.

Compound 554: LCMS: 583.3 (M+H). ¹H NMR (400 MHz, DMSO-d6): δ 10.98 (s, 1H), 8.18 (s, 1H), 7.44 (t, J=7.6 Hz, 1H), 7.34 (d, J=7.4 Hz, 1H), 7.27 (t, J=6.4 Hz, 1H), 5.10 (dd, J=13.2, 5.1 Hz, 1H), 4.39-4.29 (m, 1H), 4.23 (d, J=17.2 Hz, 1H), 3.32-3.16 (m, 3H), 3.01 (s, 1H), 2.96-2.84 (m, 1H), 2.75 (tdt, J=11.5, 7.6, 3.7 Hz, 2H), 2.60 (ddd, J=17.2, 4.4, 2.1 Hz, 1H), 2.48-2.30 (m, 3H), 2.07-1.94 (m, 1H), 1.87 (d, J=12.1 Hz, 2H), 1.82-1.63 (m, 3H), 1.62-1.36 (m, 5H), 1.09 (d, J=14.2 Hz, 2H), 0.85 (t, J=3.2 Hz, 2H), 0.69 (d, J=7.7 Hz, 2H).

Compound 554A:LCMS: 583.3 (M+H). ¹H NMR (400 MHz, DMSO-d6): δ 10.98 (s, 1H), 7.44 (q, J=6.6, 5.5 Hz, 1H), 7.36 (d, J=7.3 Hz, 1H), 7.31 (d, J=7.9 Hz, 1H), 6.08 (d, J=10.0 Hz, 1H), 5.10 (dd, J=13.1, 5.2 Hz, 1H), 4.43-4.05 (m, 2H), 3.19 (q, J=13.5, 10.7 Hz, 3H), 3.02-2.84 (m, 2H), 2.68 (dddd, J=39.9, 19.5, 9.8, 4.2 Hz, 6H), 2.02 (dd, J=11.4, 5.8 Hz, 1H), 1.79 (d, J=10.7 Hz, 4H), 1.65 (d, J=12.3 Hz, 1H), 1.49 (pd, J=14.7, 14.0, 7.6 Hz, 5H), 1.24 (s, 1H), 1.10 (s, 3H), 0.91-0.79 (m, 2H), 0.70 (s, 2H).

### Compound 557

### 3-(1-oxo-4-{[(1r,4r)-4-[(3,3-difluorocyclobutyl)amino]cyclohexyl]({spiro[3.3]heptan-2-ylmethyl})amino}-3H-isoindol-2-yl)piperidine-2,6-dione

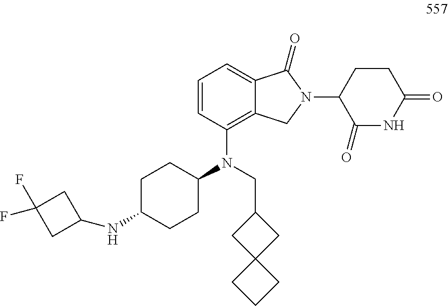 [SMILES: O=C1CCC(N2Cc3c(cccc3N(CC3CC4(CCC4)C3)[C@H]3CC[C@H](NC4CC(F)(F)C4)CC3)C2=O)C(=O)N1]

The compound was prepared by the same procedure described as preparation of Compounds 554 and 554A above.

LCMS: 555.31 (M+H). ¹H NMR (400 MHz, DMSO-d₆): 10.98 (s, 1H), 8.99 (d, J=6.0 Hz, 2H), 7.41 (d, J=7.6 Hz, 1H), 7.36 (s, 1H), 7.30 (s, 1H), 5.14-5.09 (m, 1H), 4.29 (s, 1H), 3.81-3.78 (m, 1H), 3.05-2.93 (m, 9H), 2.62 (d, J=3.7 Hz, 1H), 2.45 (d, J=4.4 Hz, 1H), 2.03-1.98 (m, 4H), 1.84-1.80 (m, 8H), 1.72-1.69 (m, 2H), 1.55-1.50 (m, 4H), 1.35-1.30 (m, 2H).

### Compound 558-2

### 3-[1-oxo-4-({[(1r,3r)-3-aminocyclobutyl]methyl}({spiro[3.3]heptan-2-ylmethyl})amino)-3H-isoindol-2-yl]piperidine-2,6-dione

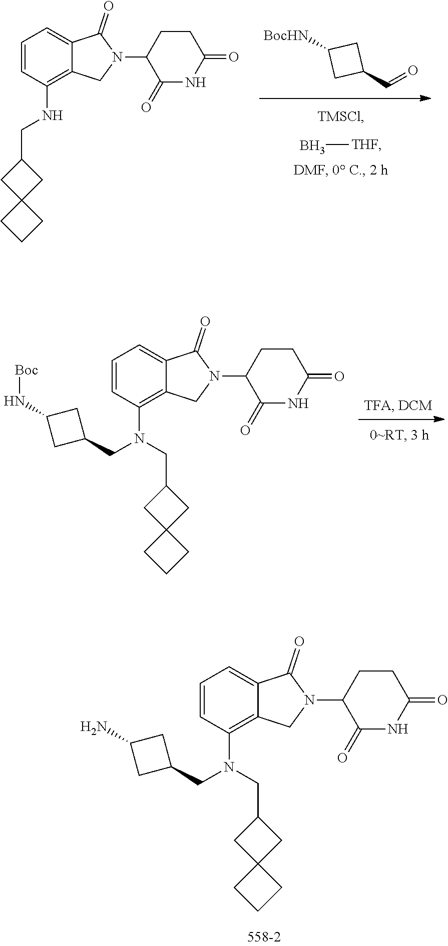

### Step-1: tert-butyl N-[(1r,3r)-3-({[2-(2,6-dioxopiperidin-3-yl)-1-oxo-3H-isoindol-4-yl]({spiro[3.3]heptan-2-ylmethyl})amino}methyl)cyclobutyl]carbamate

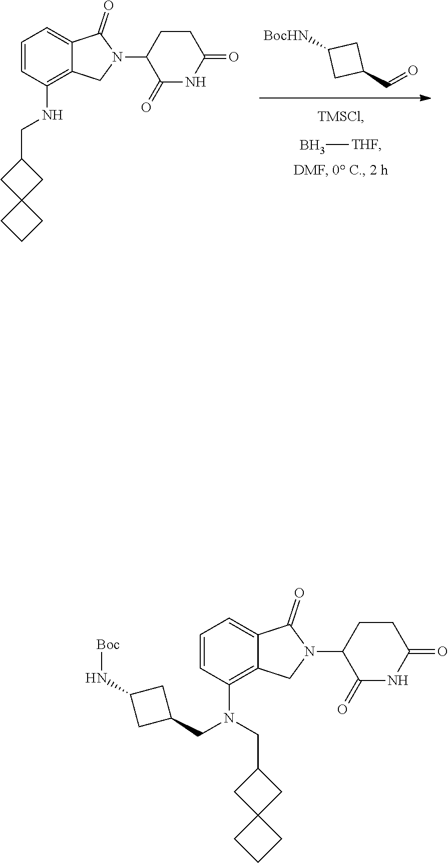

A solution of 3-[1-oxo-4-({spiro[3.3]heptan-2-ylmethyl}amino)-3H-isoindol-2-yl]piperidine-2,6-dione (100 mg, 0.27 mmol, 1 equiv) and tert-butyl N-[(1r,3r)-3-formylcyclobutyl]carbamate (81 mg, 0.41 mmol, 1.5 equiv) in DMF (2 mL) was treated with TMSCl (295 mg, 2.7 mmol, 10 equiv) for 1 h at 0° C. under nitrogen atmosphere followed by the addition of BH₃-THF (0.82 ml, 0.816 mmol, 3 equiv) dropwise at 0 degrees C. The resulting mixture was stirred for 1 h at 0° C. under nitrogen atmosphere. The reaction was quenched with MeOH at 0° C. The residue was purified by reversed-phase flash chromatography with the following conditions: column, C18 silica gel; mobile phase, MeCN in Water (0.1% FA), 0% to 100% gradient in 20 min; detector, UV 254 nm. This resulted in tert-butyl N-[(1r,3r)-3-({[2-(2,6-dioxopiperidin-3-yl)-1-oxo-3H-isoindol-4-yl]({spiro[3.3]heptan-2-ylmethyl})amino}methyl)cyclobutyl]carbamate (130 mg, 87%) as an off-white solid.

### Step-2: 3-[1-oxo-4-({[(1r,3r)-3-aminocyclobutyl]methyl}({spiro[3.3]heptan-2-ylmethyl})amino)-3H-isoindol-2-yl]piperidine-2,6-dione

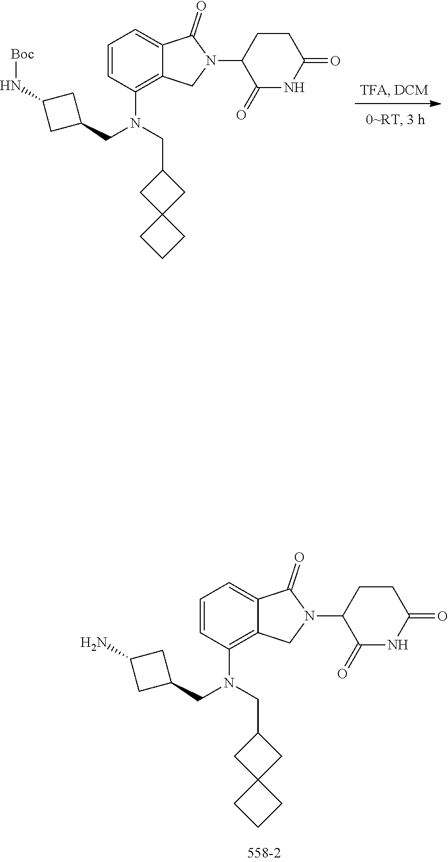 [SMILES: CC(C)(C)OC(=O)N[C@H]1C[C@H](CN(CC2CC3(CCC3)C2)c2cccc3c2CN(C2CCC(=O)NC2=O)C3=O)C1 ; N[C@H]1C[C@H](CN(CC2CC3(CCC3)C2)c2cccc3c2CN(C2CCC(=O)NC2=O)C3=O)C1]

To a stirred solution of tert-butyl N-[(1r,3r)-3-({[2-(2,6-dioxopiperidin-3-yl)-1-oxo-3H-isoindol-4-yl]({spiro[3.3]heptan-2-ylmethyl})amino}methyl)cyclobutyl]carbamate (140 mg, 0.25 mmol, 1 equiv) in DCM (3 mL) was added TFA (0.6 mL, 8.1 mmol, 31 equiv) dropwise at 0° C. under nitrogen atmosphere. The resulting mixture was stirred for 3 h at room temperature under nitrogen atmosphere. The resulting mixture was concentrated under reduced pressure. The residue was purified by reversed-phase flash chromatography with the following conditions: column, C18 silica gel; mobile phase, MeCN in Water (0.1% FA), 0% to 100% gradient in 20 min; detector, UV 254 nm. This resulted in 3-[1-oxo-4-({[(1r,3r)-3-aminocyclobutyl]methyl}({spiro[3.3]heptan-2-ylmethyl})amino)-3H-isoindol-2-yl]piperidine-2,6-dione (84 mg, 73%) as a yellow solid.

LC-MS: (ES, m/z): [M+1]=451.25. ¹H NMR (400 MHz, DMSO-d₆): δ 7.39 (t, J=7.7 Hz, 1H), 7.27 (dd, J=7.2, 3.7 Hz, 1H), 7.16 (d, J=7.9 Hz, 1H), 5.10 (dd, J=13.2, 5.2 Hz, 1H), 4.40-4.19 (m, 2H), 3.24-3.01 (m, 4H), 2.91 (ddd, J=17.3, 13.5, 5.3 Hz, 1H), 2.60 (ddd, J=17.1, 4.5, 2.2 Hz, 1H), 2.46 (dd, J=13.2, 4.5 Hz, 1H), 2.24 (p, J=7.7 Hz, 1H), 2.15 (d, J=12.8 Hz, 2H), 2.04-1.85 (m, 6H), 1.79 (qd, J=8.2, 7.7, 1.7 Hz, 3H), 1.70 (tt, J=10.1, 6.0 Hz, 3H), 1.65-1.47 (m, 5H).

### Compound 561

### 3-{4-[(2-cyclopropylethyl)[(1r,4r)-4-{[2-(4,4-difluoropiperidin-1-yl)-2-oxoethyl]amino}cyclohexyl]amino]-1-oxo-3H-isoindol-2-yl}piperidine-2,6-dione

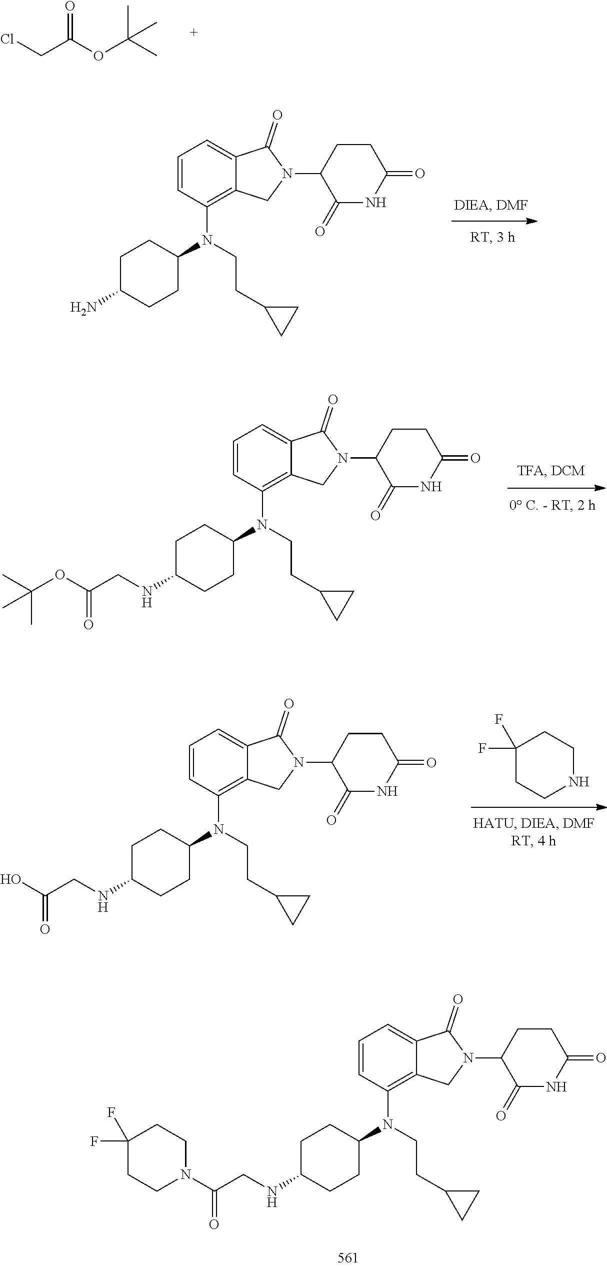

### Step-1: tert-butyl 2-{[(1r,4r)-4-[(2-cyclopropylethyl)[2-(2,6-dioxopiperidin-3-yl)-1-oxo-3H-isoindol-4-yl]amino]cyclohexyl]amino}acetate

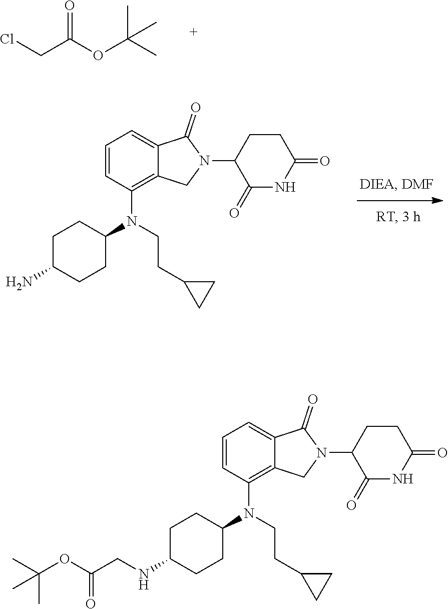

A solution of tert-butyl 2-chloroacetate (20 mg, 0.13 mmol, 0.7 equiv), 3-{4-[(2-cyclopropylethyl)[(1r,4r)-4-aminocyclohexyl]amino]-1-oxo-3H-isoindol-2-yl}piperidine-2,6-dione (80 mg, 0.188 mmol, 1.0 equiv) and DIEA (73 mg, 0.56 mmol, 3 equiv) in DMF (2.00 mL) was stirred for 3 h at room temperature under nitrogen atmosphere. The resulting mixture was diluted with water (50 mL). The resulting mixture was extracted with EtOAc (3×50 mL). The combined organic layers were washed with water (2×50 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in tert-butyl 2-{[(1r,4r)-4-[(2-cyclopropylethyl)[2-(2,6-dioxopiperidin-3-yl)-1-oxo-3H-isoindol-4-yl]amino]cyclohexyl]amino}acetate (36 mg, 35%) as a yellow oil.

### Step-2: {[(1r,4r)-4-[(2-cyclopropylethyl)[2-(2,6-dioxopiperidin-3-yl)-1-oxo-3H-isoindol-4-yl]amino]cyclohexyl]amino}acetic acid

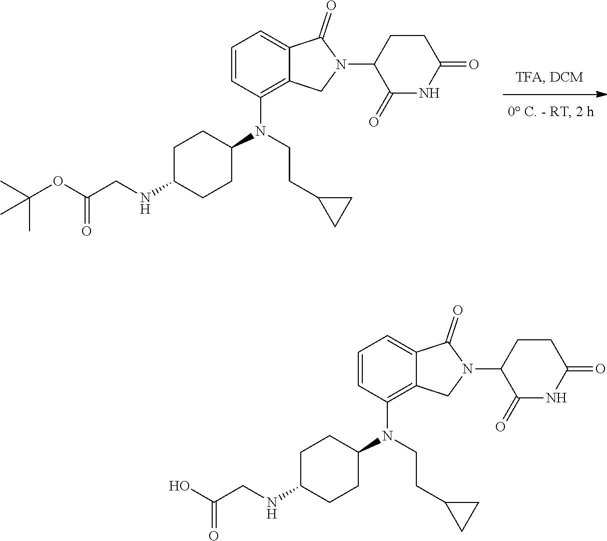 [SMILES: CC(C)(C)OC(=O)CN[C@H]1CC[C@H](N(CCC2CC2)c2cccc3c2CN(C2CCC(=O)NC2=O)C3=O)CC1 ; O=C(O)CN[C@H]1CC[C@H](N(CCC2CC2)c2cccc3c2CN(C2CCC(=O)NC2=O)C3=O)CC1]

A solution of tert-butyl 2-{[(1r,4r)-4-[(2-cyclopropylethyl)[2-(2,6-dioxopiperidin-3-yl)-1-oxo-3H-isoindol-4-yl]amino]cyclohexyl]amino}acetate (36 mg, 0.067 mmol, 1 equiv) in DCM (0.2 mL) was stirred for 10 min at 0° C. under nitrogen atmosphere. To the above mixture was added TFA (0.2 mL, 4.85 mmol, 72 equiv) dropwise at 0° C. The resulting mixture was stirred for additional 2 h at room temperature. The resulting mixture was concentrated under reduced pressure. The resulting oil was dried by lyophilization. The crude product, {[(1r,4r)-4-[(2-cyclopropylethyl)[2-(2,6-dioxopiperidin-3-yl)-1-oxo-3H-isoindol-4-yl]amino]cyclohexyl]amino}acetic acid, (50 mg) was used in the next step directly without further purification.

### Step-3: 3-{4-[(2-cyclopropylethyl)[(1r,4r)-4-{[2-(4,4-difluoropiperidin-1-yl)-2-oxoethyl]amino}cyclohexyl]amino]-1-oxo-3H-isoindol-2-yl}piperidine-2,6-dione

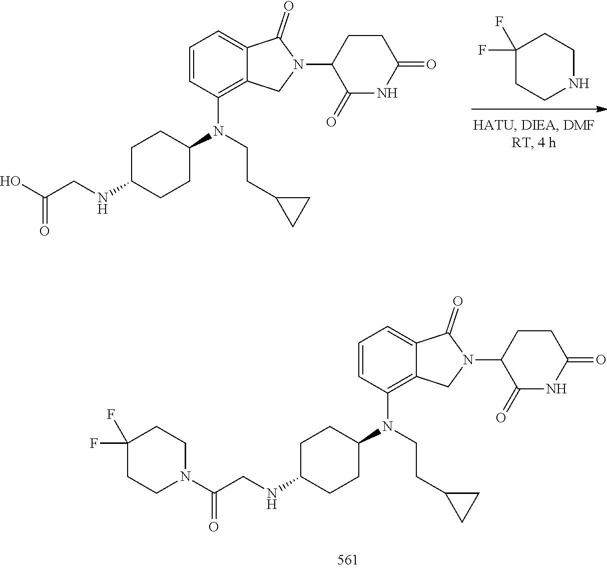 [SMILES: O=C(O)CN[C@H]1CC[C@H](N(CCC2CC2)c2cccc3c2CN(C2CCC(=O)NC2=O)C3=O)CC1 ; FC1(F)CCNCC1 ; O=C1CCC(N2Cc3c(cccc3N(CCC3CC3)[C@H]3CC[C@H](NCC(=O)N4CCC(F)(F)CC4)CC3)C2=O)C(=O)N1]

A solution of {[(1r,4r)-4-[(2-cyclopropylethyl)[2-(2,6-dioxopiperidin-3-yl)-1-oxo-3H-isoindol-4-yl]amino]cyclohexyl]amino}acetic acid (50 mg, 0.10 mmol, 1 equiv), 4,4-difluoropiperidine (51 mg, 0.42 mmol, 4 equiv), HATU (60 mg, 0.15 mmol, 1.50 equiv) and DIEA (67 mg, 0.52 mmol, 5 equiv) in DMF (1 mL) was stirred for 4 h at room temperature under nitrogen atmosphere. The resulting mixture was diluted with water (50 mL). The resulting mixture was extracted with EtOAc (4×50 mL). The combined organic layers were washed with water (2×50 mL), dried over anhydrous Na₂SO₄. After filtration, the filtrate was concentrated under reduced pressure. The crude product was purified by Prep-HPLC with the following conditions (Column: Sunfire prep C18 column, 30*150 mm, 5 μm; Mobile Phase A: Water (0.1% FA), Mobile Phase B: ACN; Flow rate: 60 mL/min; Gradient: 8% B to 25% B in 9 min; Wave Length: 254/220 nm; RT1 (min): 10.75) to afford 3-{4-[(2-cyclopropylethyl)[(1r,4r)-4-{[2-(4,4-difluoropiperidin-1-yl)-2-oxoethyl]amino}cyclohexyl]amino]-1-oxo-3H-isoindol-2-yl}piperidine-2,6-dione (7.8 mg, 12%) as a white solid.

LC-MS: (ES, m/z): [M+1]=586.3; ¹H NMR (400 MHz, DMSO-d₆) δ 11.04 (s, 1H), 7.47 (t, J=7.6 Hz, 1H), 7.32 (dd, J=17.8, 7.7 Hz, 2H), 5.16 (dd, J=13.2, 5.1 Hz, 1H), 4.41 (d, J=17.2 Hz, 1H), 4.31 (d, J=17.1 Hz, 1H), 3.64 (d, J=9.3 Hz, 4H), 3.29-3.20 (m, 3H), 3.15 (s, 2H), 3.02-2.91 (m, 1H), 2.68 (t, J=3.4 Hz, 1H), 2.63 (t, J=6.4 Hz, 1H), 2.51 (t, J=9.2 Hz, 1H), 2.23-1.88 (m, 7H), 1.88-1.75 (m, 2H), 1.57 (t, J=9.5 Hz, 2H), 1.30 (s, 2H), 1.22 (dq, J=14.2, 8.6, 7.2 Hz, 4H), 0.69 (h, J=6.5 Hz, 1H), 0.44-0.32 (m, 2H).

### Compound 562

### N,N-diethyl-2-{[(1r,4r)-4-[(2-cyclopropylethyl)[2-(2,6-dioxopiperidin-3-yl)-1-oxo-3H-isoindol-4-yl]amino]cyclohexyl]amino}acetamide

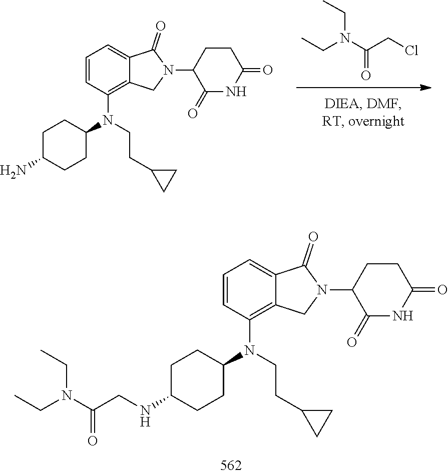

A solution of 3-{4-[(2-cyclopropylethyl)[(1r,4r)-4-aminocyclohexyl]amino]-1-oxo-3H-isoindol-2-yl}piperidine-2,6-dione (120 mg, 0.283 mmol, 1 equiv), 2-chloro-N,N-diethylacetamide (253 mg, 1.69 mmol, 6 equiv) and DIEA (110 mg, 0.849 mmol, 3 equiv) in DMF (3.6 mL) was stirred for overnight at room temperature under nitrogen atmosphere. The resulting mixture was diluted with water (100 mL). The resulting mixture was extracted with EtOAc (3×100 mL). The combined organic layers were washed with water (4×100 mL), dried over anhydrous Na₂SO₄. After filtration, the filtrate was concentrated under reduced pressure. The crude product was purified by Prep-HPLC with the following conditions (Column: Xselect CSH C18 OBD Column 30*150 mm 5 μm, n; Mobile Phase A: Water (0.1% FA), Mobile Phase B: MeOH—HPLC; Flow rate: 60 mL/min; Gradient: 28% B to 41% B in 9 min; Wave Length: 254/220 nm; RT1 (min): 8.25) to afford N,N-diethyl-2-{[(1r,4r)-4-[(2-cyclopropylethyl)[2-(2,6-dioxopiperidin-3-yl)-1-oxo-3H-isoindol-4-yl]amino]cyclohexyl]amino}acetamide (7.8 mg, 5%) as a white solid.

LC-MS: (ES, m/z): [M+1]=538.40; ¹H NMR (400 MHz, Methanol-d₄) δ 8.54 (s, 1H), 7.59-7.49 (m, 2H), 7.45 (p, J=3.9 Hz, 1H), 5.21 (dd, J=13.3, 5.2 Hz, 1H), 4.50 (s, 2H), 4.04 (d, J=2.7 Hz, 2H), 3.50 (q, J=7.1 Hz, 2H), 3.42 (dd, J=7.1, 1.9 Hz, 2H), 3.33 (td, J=7.2, 3.0 Hz, 2H), 3.20 (t, J=10.5, 3.5 Hz, 1H), 3.15-3.04 (m, 1H), 3.03-2.90 (m, 1H), 2.85 (ddd, J=17.6, 4.7, 2.4 Hz, 1H), 2.62 (qd, J=13.3, 4.8 Hz, 1H), 2.25 (ddt, J=12.9, 5.5, 2.7 Hz, 3H), 2.07 (ddd, J=13.7, 6.5, 3.3 Hz, 2H), 1.70 (qt, J=12.7, 3.2 Hz, 2H), 1.54 (q, J=12.7 Hz, 2H), 1.41-1.36 (m, 1H), 1.29 (t, J=7.0 Hz, 6H), 1.20 (q, J=7.0 Hz, 4H), 0.72 (dqd, J=14.9, 7.3, 4.9 Hz, 1H), 0.49-0.38 (m, 2H), −0.06 (s, 2H).

### Compound 573

### 3-{4-[(2-cyclopropylethyl)[(1s,4s)-4-(4H-1,2,4-triazol-3-yl)cyclohexyl]amino]-1-oxo-3H-isoindol-2-yl}piperidine-2,6-dione; formate

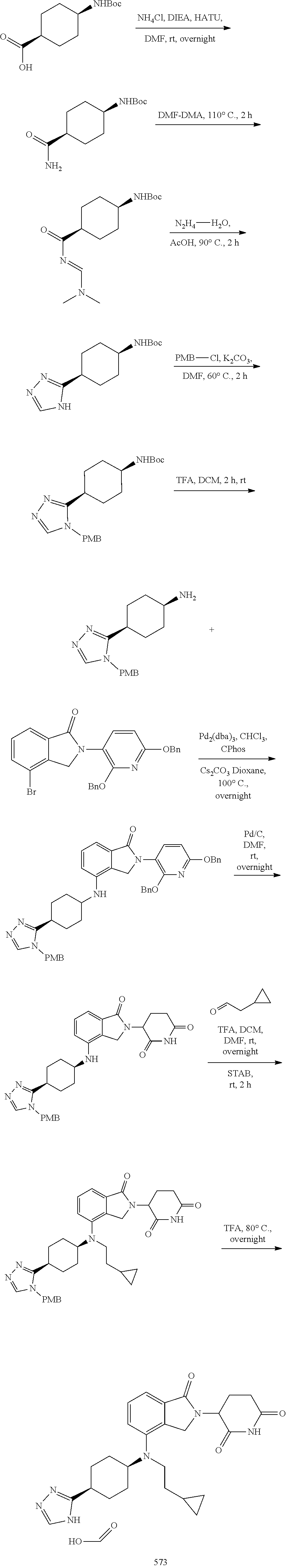

### Step-1: tert-butyl N-[(1s,4s)-4-carbamoylcyclohexyl]carbamate

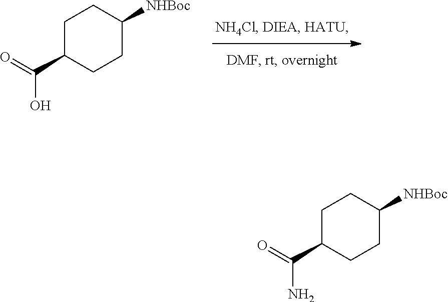 [SMILES: CC(C)(C)OC(=O)N[C@H]1CC[C@@H](C(=O)O)CC1 ; CC(C)(C)OC(=O)N[C@H]1CC[C@@H](C(N)=O)CC1]

To a stirred solution of (1s,4s)-4-[(tert-butoxycarbonyl)amino]cyclohexane-1-carboxylic acid (3.8 g, 15.6 mmol, 1 equiv) and NH₄Cl (2.5 g, 46.8 mmol, 3 equiv) in DMF (38 mL) were added DIEA (3.0 g, 23.4 mmol, 1.5 equiv) and HATU (17.8 g, 46.8 mmol, 3 equiv) in portions at room temperature under nitrogen atmosphere. The resulting mixture was stirred for overnight at room temperature under nitrogen atmosphere. The resulting mixture was diluted with water (200 mL) and extracted with EtOAc (3×200 mL). The combined organic layers were washed with brine (3×200 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% NH₃·H₂O), 0% to 100% gradient in 10 min; detector, UV 254 nm to afford tert-butyl N-[(1s,4s)-4-carbamoylcyclohexyl]carbamate (2.8 g, 74%) as an off-white solid.

### Step-2: N-[(1s,4s)-4-{[(1Z)-(dimethylamino)methylidene]carbamoyl}cyclohexyl]carbamate

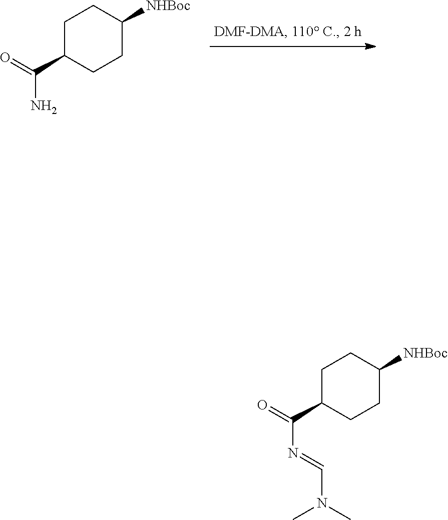 [SMILES: CC(C)(C)OC(=O)N[C@H]1CC[C@@H](C(N)=O)CC1 ; CN(C)/C=N/C(=O)[C@H]1CC[C@@H](NC(=O)OC(C)(C)C)CC1]

A solution of tert-butyl N-[(1s,4s)-4-carbamoylcyclohexyl]carbamate (2.8 g, 11.5 mmol, 1 equiv) in DMF-DMA (60 mL) was stirred for 2 h at 110° C. under nitrogen atmosphere. The resulting mixture was diluted with water (300 mL) and extracted with EtOAc (3×200 mL). The combined organic layers were washed with brine (3×300 mL), dried over anhydrous Na₂SO₄. After filtration, the filtrate was concentrated under reduced pressure. The crude product, N-[(1s,4s)-4-{[(1Z)-(dimethylamino)methylidene]carbamoyl}cyclohexyl]carbamate, was used in the next step directly without further purification.

### Step-3: tert-butyl N-[(1s,4s)-4-(4H-1,2,4-triazol-3-yl)cyclohexyl]carbamate

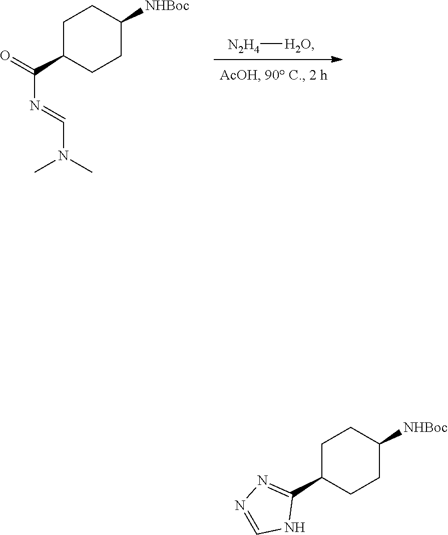

A solution of tert-butyl N-[(1s,4s)-4-{[(1Z)-(dimethylamino)methylidene]carbamoyl}cyclohexyl]carbamate (2.8 g, 9.4 mmol, 1 equiv) and hydrazine hydrate (85%) (6 mL, 123 mmol, 13 equiv) in AcOH (60 mL) was stirred for 2 h at 90° C. under nitrogen atmosphere. The resulting mixture was diluted with water (200 mL) and extracted with EtOAc (3×200 mL). The combined organic layers were washed with NaHCO₃ (aq.) (3×500 mL), dried over anhydrous Na₂SO₄. After filtration, the filtrate was concentrated under reduced pressure. The crude product, tert-butyl N-[(1s,4s)-4-(4H-1,2,4-triazol-3-yl)cyclohexyl]carbamate, was used in the next step directly without further purification.

### Step-4: tert-butyl N-[(1s,4s)-4-{4-[(4-methoxyphenyl)methyl]-1,2,4-triazol-3-yl}cyclohexyl]carbamate

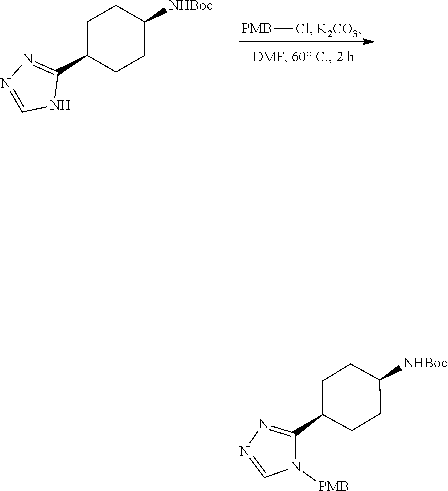

To a stirred solution of tert-butyl N-[(1s,4s)-4-(4H-1,2,4-triazol-3-yl)cyclohexyl]carbamate (3.0 g, 11.3 mmol, 1 equiv) and K₂CO₃ (4.7 g, 33.8 mmol, 3 equiv) in DMF (60 mL) was added 4-methoxybenzyl chloride (3.5 g, 22.5 mmol, 2 equiv) at room temperature. The resulting mixture was stirred for 2 h at 60° C. The resulting mixture was diluted with water (200 mL) and extracted with EtOAc (3×300 mL). The combined organic layers were washed with brine (3×300 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 0% to 100% gradient in 10 min; detector, UV 254 nm to afford tert-butyl N-[(1s,4s)-4-{4-[(4-methoxyphenyl)methyl]-1,2,4-triazol-3-yl}cyclohexyl]carbamate (2.7 g, 62%) as an off-white solid.

### Step-5: (1s,4s)-4-{4-[(4-methoxyphenyl)methyl]-1,2,4-triazol-3-yl}cyclohexan-1-amine

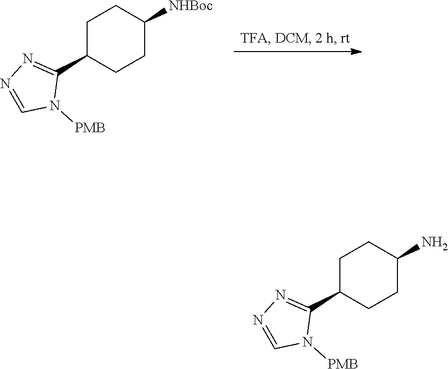

To a solution of tert-butyl N-[(1s,4s)-4-{4-[(4-methoxyphenyl)methyl]-1,2,4-triazol-3-yl}cyclohexyl]carbamate (2.7 g, 6.8 mmol, 1 equiv) in DCM (40 mL) was added TFA (13 mL, 175 mmol, 25 equiv) dropwise at 0° C. The resulting mixture was stirred for 2 h at room temperature. The resulting mixture was concentrated under reduced pressure. The residue was purified by reversed-phase flash chromatography with the following conditions: column, C18 silica gel; mobile phase, MeCN in Water (0.1% NH₃·H₂O), 0% to 100% gradient in 10 min; detector, UV 254 nm to afford (1s,4s)-4-{4-[(4-methoxyphenyl)methyl]-1,2,4-triazol-3-yl}cyclohexan-1-amine (600 mg, 31%) as a light yellow solid.

### Step-6:2-[2,6-bis(benzyloxy)pyridin-3-yl]-4-{[(1s,4s)-4-{4-[(4-methoxyphenyl)methyl]-1,2,4-triazol-3-yl}cyclohexyl]amino}-3H-isoindol-1-one

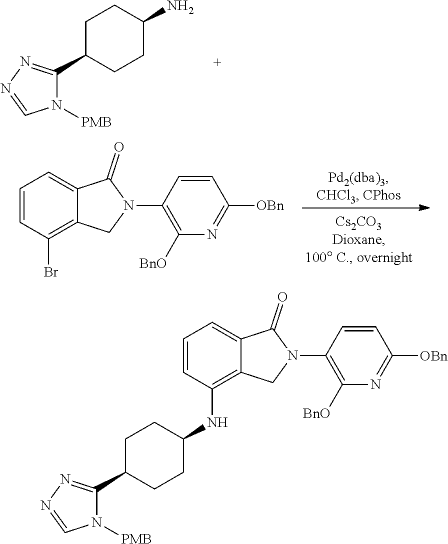

To a stirred solution of 2-[2,6-bis(benzyloxy)pyridin-3-yl]-4-bromo-3H-isoindol-1-one (450 mg, 0.89 mmol, 1 equiv) and (1s,4s)-4-{4-[(4-methoxyphenyl)methyl]-1,2,4-triazol-3-yl}cyclohexan-1-amine (514 mg, 1.79 mmol, 2 equiv) in dioxane (9 mL) were added Cs₂CO₃ (877 mg, 2.69 mmol, 3 equiv), CPhos (78 mg, 0.18 mmol, 0.2 equiv) and tris((1E,4E)-1,5-diphenylpenta-1,4-dien-3-one) trichloromethane dipalladium (92.9 mg, 0.09 mmol, 0.10 equiv) at room temperature under nitrogen atmosphere. The resulting mixture was stirred for overnight at 100° C. under nitrogen atmosphere. The resulting mixture was diluted with water (100 mL). The resulting mixture was extracted with EtOAc (3×100 mL). The combined organic layers were washed with brine (3×300 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 0% to 100% gradient in 10 min; detector, UV 254 nm to afford 2-[2,6-bis(benzyloxy)pyridin-3-yl]-4-{[(1s,4s)-4-{4-[(4-methoxyphenyl)methyl]-1,2,4-triazol-3-yl}cyclohexyl]amino}-3H-isoindol-1-one (390 mg, 61%) as a light yellow solid.

### Step-7:3-(1-oxo-4-{[(1s,4s)-4-{4-[(4-methoxyphenyl)methyl]-1,2,4-triazol-3-yl}cyclohexyl]amino}-3H-isoindol-2-yl)piperidine-2,6-dione

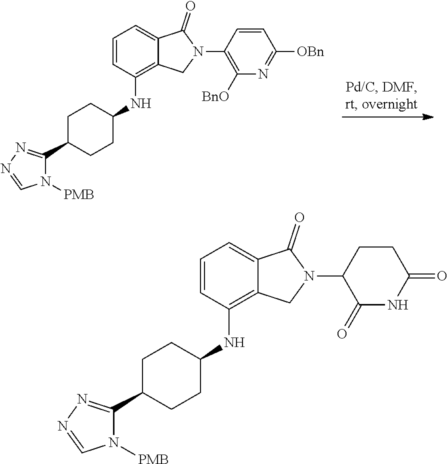

To a stirred solution of 2-[2,6-bis(benzyloxy)pyridin-3-yl]-4-{[(1s,4s)-4-{4-[(4-methoxyphenyl)methyl]-1,2,4-triazol-3-yl}cyclohexyl]amino}-3H-isoindol-1-one (380 mg, 0.53 mmol, 1 equiv) in DMF (8 mL) was added palladium 10% on carbon (wetted with ca. 55% water) (600 mg, 0.564 mmol, 1 equiv, 10%) at room temperature under nitrogen atmosphere. The resulting mixture was stirred for overnight at room temperature under hydrogen atmosphere. The resulting mixture was filtered, the filter cake was washed with DCM (3×5 mL). The filtrate was concentrated under reduced pressure. The residue was purified by reversed-phase flash chromatography with the following conditions: column, C18 silica gel; mobile phase, MeCN in Water (0.1% FA), 0% to 100% gradient in 10 min; detector, UV 254 nm to afford 3-(1-oxo-4-{[(1s,4s)-4-{4-[(4-methoxyphenyl)methyl]-1,2,4-triazol-3-yl}cyclohexyl]amino}-3H-isoindol-2-yl)piperidine-2,6-dione (270 mg, 95%) as a light pink solid.

### Step-8: 3-{4-[(2-cyclopropylethyl)[(1s,4s)-4-{4-[(4-methoxyphenyl)methyl]-1,2,4-triazol-3-yl}cyclohexyl]amino]-1-oxo-3H-isoindol-2-yl}piperidine-2,6-dione

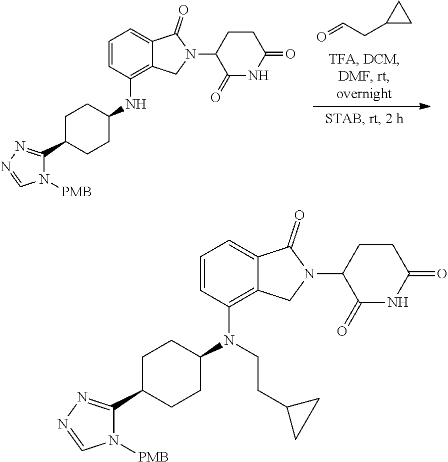

To a stirred solution of 3-(1-oxo-4-{[(1s,4s)-4-{4-[(4-methoxyphenyl)methyl]-1,2,4-triazol-3-yl}cyclohexyl]amino}-3H-isoindol-2-yl)piperidine-2,6-dione (250 mg, 0.47 mmol, 1 equiv) and 2-cyclopropylacetaldehyde (795 mg, 9.46 mmol, 20 equiv) in DCM (6.25 mL) and DMF (1.50 mL) was added TFA (162 mg, 1.4 mmol, 3 equiv) at room temperature under nitrogen atmosphere. The resulting mixture was stirred for overnight at room temperature under nitrogen atmosphere. To the above mixture was added STAB (501 mg, 2.4 mmol, 5 equiv) in portions over 2 min at 0° C. The resulting mixture was stirred for additional 2 h at room temperature. The resulting mixture was diluted with water (50 mL) and extracted with EtOAc (3×30 mL). The combined organic layers were washed with brine (3×100 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 0% to 100% gradient in 10 min; detector, UV 254 nm to afford 3-{4-[(2-cyclopropylethyl)[(1s,4s)-4-{4-[(4-methoxyphenyl)methyl]-1,2,4-triazol-3-yl}cyclohexyl]amino]-1-oxo-3H-isoindol-2-yl}piperidine-2,6-dione (200 mg, 71%) as a light yellow oil.

### Step-9: 3-{4-[(2-cyclopropylethyl)[(1s,4s)-4-(4H-1,2,4-triazol-3-yl)cyclohexyl]amino]-1-oxo-3H-isoindol-2-yl}piperidine-2,6-dione; formic acid

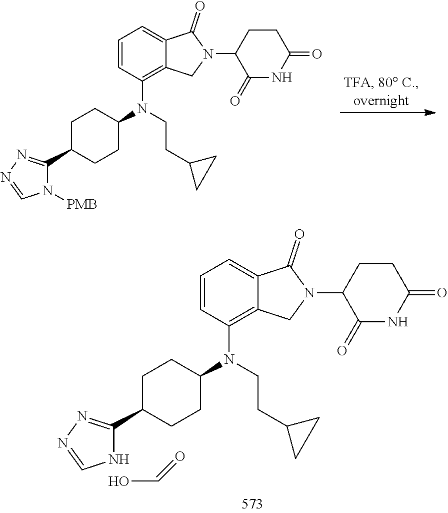

A solution of 3-{4-[(2-cyclopropylethyl)[(1s,4s)-4-{4-[(4-methoxyphenyl)methyl]-1,2,4-triazol-3-yl}cyclohexyl]amino]-1-oxo-3H-isoindol-2-yl}piperidine-2,6-dione (190 mg, 0.32 mmol, 1 equiv) in TFA (3.8 mL, 51 mmol, 160 equiv) was stirred for overnight at 80° C. The resulting mixture was concentrated under reduced pressure. The crude product (200 mg) was purified by Prep-HPLC with the following conditions (Column: XSelect CSH Fluoro Phenyl 30*150 mm, 5 m; Mobile Phase A: Water (0.1% FA), Mobile Phase B: 20 mm NaOH+10% ACN; Flow rate: 60 mL/min mL/min; Gradient: isocratic 2% B t20% B in 10 min; Wave Length: 254 nm/220 nm; RT1 (min): 12.78) to afford 3-{4-[(2-cyclopropylethyl)[(1s,4s)-4-(4H-1,2,4-triazol-3-yl)cyclohexyl]amino]-1-oxo-3H-isoindol-2-yl}piperidine-2,6-dione; formic acid (27.7 mg, 17%) as an off-white solid.

LCMS: 477.2 (M+H). ¹H NMR (400 MHz, DMSO-d₆): δ 13.73 (s, 1H), 11.08 (s, 1H), 8.35 (s, 1H), 7.51 (t, J=7.6 Hz, 1H), 7.37 (dd, J=20.1, 7.6 Hz, 2H), 5.21 (dd, J=13.2, 5.1 Hz, 1H), 4.53-4.32 (m, 2H), 3.36 (s, 1H), 3.20 (h, J=6.8 Hz, 2H), 3.12 (s, 1H), 3.04-2.95 (m, 1H), 2.76-2.63 (m, 2H), 2.34-2.18 (m, 2H), 2.12 (tt, J=6.8, 4.3 Hz, 1H), 1.83 (d, J=9.5 Hz, 4H), 1.70 (s, 2H), 1.35 (s, 1H), 1.23 (q, J=7.1 Hz, 2H), 0.68 (td, J=10.1, 8.0, 5.5 Hz, 1H), 0.48-0.36 (m, 2H), −0.01 (q, J=4.8 Hz, 2H).

### Compound 574

### 3-{4-[(2-cyclopropylethyl)[(1r,4r)-4-(4H-1,2,4-triazol-3-yl)cyclohexyl]amino]-1-oxo-3H-isoindol-2-yl}piperidine-2,6-dione; formate

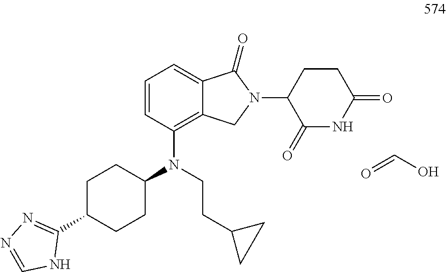 [SMILES: O=C1CCC(N2Cc3c(cccc3N(CCC3CC3)[C@H]3CC[C@H](c4nnc[nH]4)CC3)C2=O)C(=O)N1 ; O=CO]

The compound was prepared by the same procedure described as preparation of Compound 573 above.

LC-MS: (ES, m/z): [M+1]=477; ¹H NMR (400 MHz, DMSO-d6) δ 13.63 (s, 1H), 11.01 (s, 1H), 8.43-7.78 (m, 1H), 7.46 (t, J=7.7 Hz, 1H), 7.32 (t, J=8.2 Hz, 2H), 5.14 (dd, J=13.1, 5.1 Hz, 1H), 4.42 (d, J=17.2 Hz, 1H), 4.32 (d, J=17.2 Hz, 1H), 3.35-3.26 (m, 3H), 3.26-3.16 (m, 1H), 2.94 (ddd, J=16.7, 13.4, 5.3 Hz, 1H), 2.76 (s, 1H), 2.69-2.55 (m, 2H), 2.11-2.00 (m, 3H), 1.90 (d, J=11.3 Hz, 2H), 1.79-1.65 (m, 2H), 1.63-1.51 (m, 2H), 1.32-1.17 (m, 2H), 0.76-0.64 (m, 1H), 0.43-0.34 (m, 2H), −0.06 (s, 2H).

### Compound 575

### 3-{4-[(2-cyclopropylethyl)[(1s,4s)-4-(pyridin-2-ylamino)cyclohexyl]amino]-1-oxo-3H-isoindol-2-yl}piperidine-2,6-dione

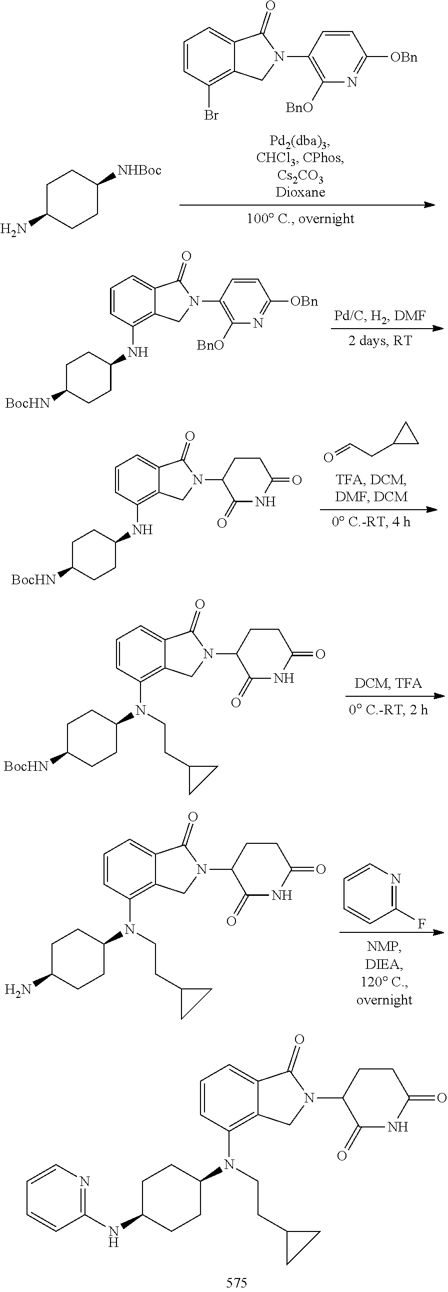

### Step-1: tert-butyl N-[(1s,4s)-4-({2-[2,6-bis(benzyloxy)pyridin-3-yl]-1-oxo-3H-isoindol-4-yl}amino)cyclohexyl]carbamate

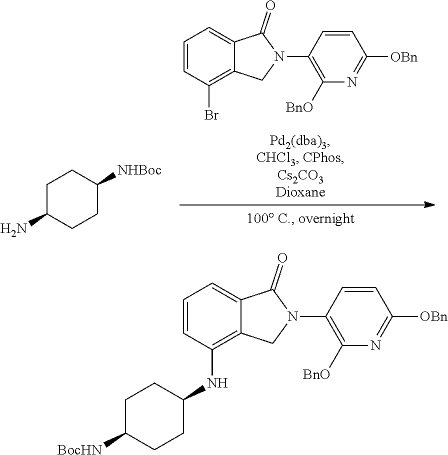 [SMILES: O=C1c2cccc(Br)c2CN1c1ccc(OCc2ccccc2)nc1OCc1ccccc1 ; CC(C)(C)OC(=O)N[C@H]1CC[C@@H](N)CC1 ; CC(C)(C)OC(=O)N[C@H]1CC[C@@H](Nc2cccc3c2CN(c2ccc(OCc4ccccc4)nc2OCc2ccccc2)C3=O)CC1]

To a stirred solution of tert-butyl N-[(1s,4s)-4-aminocyclohexyl]carbamate (1 g, 4.7 mmol, 1 equiv) and 2-[2,6-bis(benzyloxy)pyridin-3-yl]-4-bromo-3H-isoindol-1-one (2.8 g, 5.59 mmol, 1.2 equiv) in Dioxane (20 mL) was added Cs₂CO₃ (4.56 g, 13.9 mmol, 3 equiv) at room temperature. The resulting mixture was stirred for 10 min at room temperature under nitrogen atmosphere. To the above mixture was added Pd₂(dba)₃·CHCl₃ (0.48 g, 0.46 mmol, 0.1 equiv) and CPhos (0.41 g, 0.93 mmol, 0.2 equiv) at room temperature. The resulting mixture was stirred for additional overnight at 100° C. The resulting mixture was diluted with water (100 mL) and extracted with EtOAc (3×100 mL). The combined organic layers were washed with brine (2×50 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 0% to 100% gradient in 20 min; detector, UV 254 nm to afford tert-butyl N-[(1s,4s)-4-({2-[2,6-bis(benzyloxy)pyridin-3-yl]-1-oxo-3H-isoindol-4-yl}amino)cyclohexyl]carbamate (2.5 g, 84%) as an off-white solid.

### Step-2: tert-butyl N-[(1s,4s)-4-{[2-(2,6-dioxopiperidin-3-yl)-1-oxo-3H-isoindol-4-yl]amino}cyclohexyl]carbamate

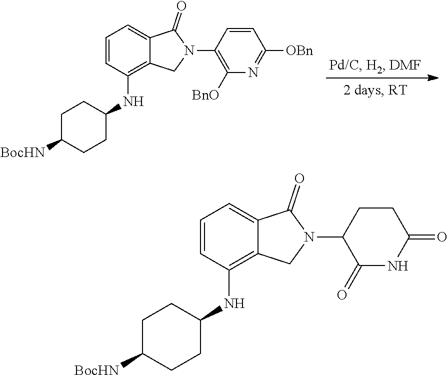 [SMILES: CC(C)(C)OC(=O)N[C@H]1CC[C@@H](Nc2cccc3c2CN(c2ccc(OCc4ccccc4)nc2OCc2ccccc2)C3=O)CC1 ; CC(C)(C)OC(=O)N[C@H]1CC[C@@H](Nc2cccc3c2CN(C2CCC(=O)NC2=O)C3=O)CC1]

A solution of tert-butyl N-[(1s,4s)-4-({2-[2,6-bis(benzyloxy)pyridin-3-yl]-1-oxo-3H-isoindol-4-yl}amino)cyclohexyl]carbamate (1.5 g, 2.36 mmol, 1 equiv) and Palladium 10% on Carbon (wetted with ca. 55% Water) (1.5 g, 14.1 mmol, 5.9 equiv) in DMF (30 mL) was stirred for 2 days at room temperature under hydrogen atmosphere. The resulting mixture was filtered, the filter cake was washed with THF (2×10 mL). The filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm to afford tert-butyl N-[(1s,4s)-4-{[2-(2,6-dioxopiperidin-3-yl)-1-oxo-3H-isoindol-4-yl]amino}cyclohexyl]carbamate (800 mg, 74%) as an off-white solid.

### Step-3: tert-butyl N-[(1s,4s)-4-[(2-cyclopropylethyl)[2-(2,6-dioxopiperidin-3-yl)-1-oxo-3H-isoindol-4-yl]amino]cyclohexyl]carbamate

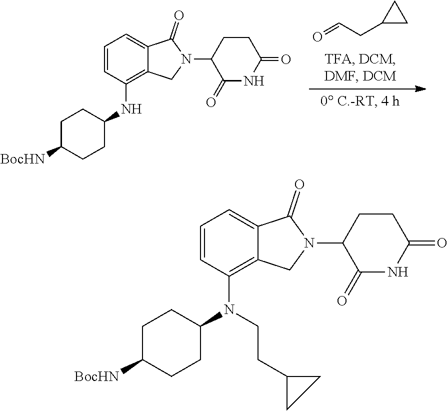

To a stirred solution of tert-butyl N-[(1s,4s)-4-{[2-(2,6-dioxopiperidin-3-yl)-1-oxo-3H-isoindol-4-yl]amino}cyclohexyl]carbamate (800 mg, 1.75 mmol, 1 equiv) and 2-cyclopropylacetaldehyde (2.95 g, 35.0 mmol, 20 equiv) in DCM (20 mL) and DMF (4.8 mL) was added TFA (600 mg, 5.25 mmol, 3 equiv) dropwise at room temperature. The resulting mixture was stirred for 2 h at room temperature under nitrogen atmosphere. To the above mixture was added STAB (1.86 g, 8.76 mmol, 5 equiv) in portions over 10 min at 0° C. The resulting mixture was stirred for additional 1.5 h at room temperature. The reaction was quenched with water (50 mL) at 0° C. The resulting mixture was extracted with EtOAc (3×200 mL). The combined organic layers were washed with brine (3×100 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 0% to 100% gradient in 15 min; detector, UV 254 nm to afford tert-butyl N-[(1s,4s)-4-[(2-cyclopropylethyl)[2-(2,6-dioxopiperidin-3-yl)-1-oxo-3H-isoindol-4-yl]amino]cyclohexyl]carbamate (180 mg, 19%) as an off-white solid.

### Step-4:3-{4-[(2-cyclopropylethyl)[(1s,4s)-4-aminocyclohexyl]amino]-1-oxo-3H-isoindol-2-yl}piperidine-2,6-dione

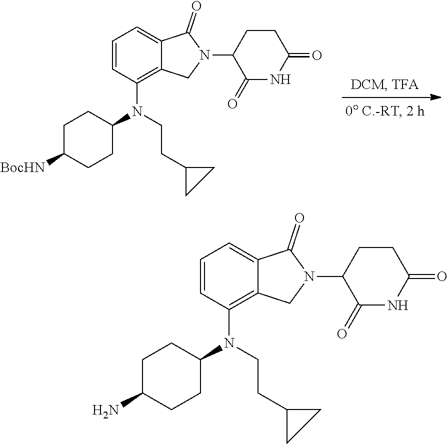 [SMILES: CC(C)(C)OC(=O)N[C@H]1CC[C@@H](N(CCC2CC2)c2cccc3c2CN(C2CCC(=O)NC2=O)C3=O)CC1 ; N[C@H]1CC[C@@H](N(CCC2CC2)c2cccc3c2CN(C2CCC(=O)NC2=O)C3=O)CC1]

A solution of tert-butyl N-[(1s,4s)-4-[(2-cyclopropylethyl)[2-(2,6-dioxopiperidin-3-yl)-1-oxo-3H-isoindol-4-yl]amino]cyclohexyl]carbamate (180 mg, 0.34 mmol, 1 equiv) in DCM (2.7 mL) was treated with TFA (0.9 mL, 12.1 mmol, 35 equiv) for 2 min at 0° C. The resulting mixture was stirred for 2 h at room temperature. The resulting mixture was concentrated under reduced pressure. This resulted in 3-{4-[(2-cyclopropylethyl)[(1s,4s)-4-aminocyclohexyl]amino]-1-oxo-3H-isoindol-2-yl}piperidine-2,6-dione (170 mg, crude) as a yellow solid. The crude product was used in the next step directly without further purification.

### Step-5: 3-{4-[(2-cyclopropylethyl)[(1s,4s)-4-(pyridin-2-ylamino)cyclohexyl]amino]-1-oxo-3H-isoindol-2-yl}piperidine-2,6-dione

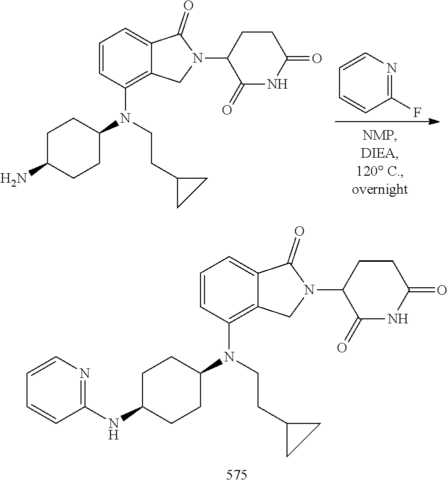

A mixture of 3-{4-[(2-cyclopropylethyl)[(1s,4s)-4-aminocyclohexyl]amino]-1-oxo-3H-isoindol-2-yl}piperidine-2,6-dione (150 mg, 0.353 mmol, 1 equiv) and DIEA (137 mg, 1.06 mmol, 3 equiv) in NMP (2.0 mL) was stirred for overnight at 120° C. under nitrogen atmosphere. The mixture was allowed to cool down to room temperature. The resulting mixture was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 6% B to 23% B in 9 min; Wave Length: 254/220 nm; RT1 (min): 9.87) to afford 3-{4-[(2-cyclopropylethyl)[(1s,4s)-4-(pyridin-2-ylamino)cyclohexyl]amino]-1-oxo-3H-isoindol-2-yl}piperidine-2,6-dione (15 mg, 8%) as a white solid.

LC-MS: (ES, m/z): [M+1]=502.3; ¹H NMR (400 MHz, DMSO-d₆) δ 11.02 (s, 1H), 7.98 (dt, J=5.2, 2.5 Hz, 1H), 7.46 (t, J=7.6 Hz, 1H), 7.41-7.28 (m, 3H), 6.63 (d, J=8.4 Hz, 1H), 6.55-6.42 (m, 2H), 5.15 (dd, J=13.1, 5.1 Hz, 1H), 4.47-4.29 (m, 2H), 4.06-3.95 (m, 1H), 3.33 (dd, J=8.9, 6.1 Hz, 2H), 3.20 (d, J=11.5 Hz, 1H), 3.00-2.88 (m, 1H), 2.69-2.59 (m, 1H), 2.11-2.01 (m, 1H), 1.88 (d, J=13.0 Hz, 4H), 1.59 (q, J=6.5, 5.5 Hz, 4H), 1.31-1.17 (m, 3H), 0.69 (qt, J=6.9, 3.6 Hz, 1H), 0.43-0.32 (m, 2H).

### Compound 576

### 3-{4-[(2-cyclopropylethyl)[(1r,4r)-4-(pyridin-2-ylamino)cyclohexyl]amino]-1-oxo-3H-isoindol-2-yl}piperidine-2,6-dione

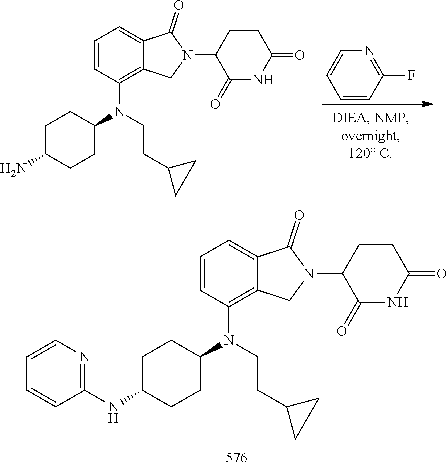

A mixture of 3-{4-[(2-cyclopropylethyl)[(1r,4r)-4-aminocyclohexyl]amino]-1-oxo-3H-isoindol-2-yl}piperidine-2,6-dione (200 mg, 0.47 mmol, 1 equiv), 2-fluoropyridine (914 mg, 9.4 mmol, 20 equiv) and DIEA (182 mg, 1.4 mmol, 3 equiv) in NMP (4 mL) was stirred for overnight at 120° C. under nitrogen atmosphere. The mixture was allowed to cool down to room temperature. The crude product was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 7% B to 24% B in 9 min; Wave Length: 254/220 nm; RT1 (min): 9.33) to afford 3-{4-[(2-cyclopropylethyl)[(1r,4r)-4-(pyridin-2-ylamino)cyclohexyl]amino]-1-oxo-3H-isoindol-2-yl}piperidine-2,6-dione (21 mg, 8%) as a white solid.

LC-MS: (ES, m/z): [M+1]=502.30; ¹H NMR (400 MHz, DMSO-d₆) δ 11.01 (s, 1H), 7.97 (dd, J=5.4, 2.0 Hz, 1H), 7.46 (t, J=7.7 Hz, 1H), 7.39-7.24 (m, 3H), 6.50-6.41 (m, 2H), 6.31 (d, J=7.6 Hz, 1H), 5.15 (dd, J=13.1, 5.1 Hz, 1H), 4.40 (d, J=17.2 Hz, 1H), 4.31 (d, J=17.1 Hz, 1H), 3.68 (dt, J=7.6, 3.9 Hz, 1H), 3.29 (t, J=7.2 Hz, 2H), 3.21-3.12 (m, 1H), 2.95 (ddd, J=17.1, 13.6, 5.5 Hz, 1H), 2.69-2.58 (m, 1H), 2.05 (ddd, J=12.8, 5.4, 2.8 Hz, 3H), 1.88-1.77 (m, 2H), 1.69 (qt, J=12.9, 3.9 Hz, 2H), 1.34-1.14 (m, 4H), 0.70 (qq, J=7.2, 4.8, 3.7 Hz, 1H), 0.42-0.33 (m, 2H).

### Compound 580

### 3-{4-[(2-cyclopropylethyl)[(1r,4r)-4-[(2,2,2-trifluoroethyl)amino]cyclohexyl]amino]-1-oxo-3H-isoindol-2-yl}piperidine-2,6-dione

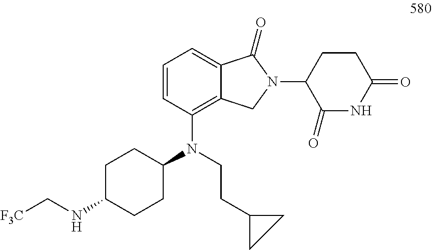 [SMILES: O=C1CCC(N2Cc3c(cccc3N(CCC3CC3)[C@H]3CC[C@H](NCC(F)(F)F)CC3)C2=O)C(=O)N1]

The compound was prepared by the general procedure described in the preparation of Compounds 515 and 515A using the appropriate amine.

LC-MS: (ES, m/z): [M+1]=507; ¹H NMR (300 MHz, DMSO-d₆) δ 10.96 (s, 1H), 7.40 (t, J=7.6 Hz, 1H), 7.24 (dd, J=14.6, 7.6 Hz, 2H), 5.09 (dd, J=13.3, 5.1 Hz, 1H), 4.34 (d, J=17.2 Hz, 1H), 4.24 (d, J=17.1 Hz, 1H), 3.19 (dd, J=11.8, 8.6 Hz, 6H), 3.08 (s, 1H), 2.99-2.81 (m, 1H), 2.59 (d, J=17.3 Hz, 1H), 2.48-2.29 (m, 2H), 2.01 (d, J=5.9 Hz, 1H), 1.90 (d, J=12.2 Hz, 2H), 1.74 (d, J=12.2 Hz, 2H), 1.50 (d, J=13.3 Hz, 2H), 1.11 (dd, J=14.4, 7.3 Hz, 3H), 1.04 (s, 1H), 0.64 (s, 1H), 0.39-0.27 (m, 2H), −0.07 (dd, J=4.8, 1.6 Hz, 2H).

### Compound 582

### 3-{4-[(2-cyclopropylethyl)[(1r,4r)-4-({[1-(trifluoromethyl)cyclopropyl]methyl}amino)cyclohexyl]amino]-1-oxo-3H-isoindol-2-yl}piperidine-2,6-dione

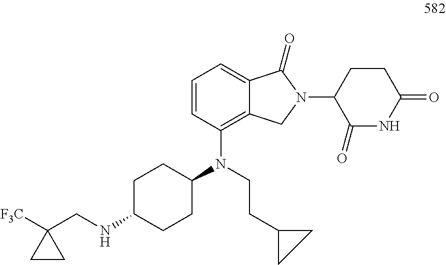 [SMILES: O=C1CCC(N2Cc3c(cccc3N(CCC3CC3)[C@H]3CC[C@H](NCC4(C(F)(F)F)CC4)CC3)C2=O)C(=O)N1]

The compound was prepared by the general procedure described in the preparation of Compounds 515 and 515A using the appropriate amine.

LCMS: 547.3 (M+H). ¹H NMR (400 MHz, D₂O): δ 8.11 (dd, J=7.2, 1.1 Hz, 1H), 8.01-7.88 (m, 2H), 5.29 (dd, J=13.3, 5.3 Hz, 1H), 4.82-4.67 (m, 2H), 3.92 (q, J=12.8, 10.3 Hz, 3H), 3.42 (s, 2H), 3.25 (t, J=11.7 Hz, 1H), 3.12-2.89 (m, 2H), 2.63 (qd, J=12.9, 5.3 Hz, 1H), 2.45-2.24 (m, 5H), 1.68 (dq, J=25.6, 12.5, 12.1 Hz, 4H), 1.44-1.21 (m, 4H), 1.06 (d, J=6.1 Hz, 2H), 0.65 (qd, J=7.3, 3.9 Hz, 1H), 0.54-0.38 (m, 2H), −0.01 (q, J=4.8 Hz, 2H).

### Compound 583

### 3-{4-[(2-cyclopropylethyl)[(1r,4r)-4-[(1,1-difluoropropan-2-yl)amino]cyclohexyl]amino]-1-oxo-3H-isoindol-2-yl}piperidine-2,6-dione

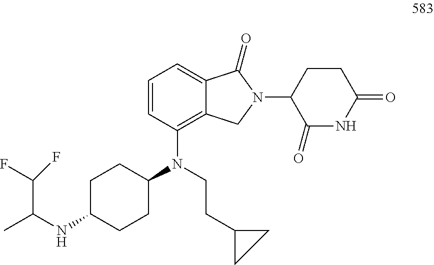 [SMILES: CC(N[C@H]1CC[C@H](N(CCC2CC2)c2cccc3c2CN(C2CCC(=O)NC2=O)C3=O)CC1)C(F)F]

The compound was prepared by the same procedure described in the preparation of Compound 540 above using the appropriate aldehyde or ketone.

LC-MS: (ES, m/z): [M+1]=503.35; ¹H-NMR (400 MHz, DMSO-d₆) δ 10.97 (s, 1H), 8.97 (d, J=29.6 Hz, 2H), 7.43 (t, J=7.7 Hz, 1H), 7.30 (dd, J=14.8, 7.6 Hz, 2H), 6.34 (td, J=54.0, 2.5 Hz, 1H), 5.13 (dd, J=13.2, 5.1 Hz, 1H), 4.36-4.20 (m, 2H), 3.87 (s, 1H), 3.21 (t, J=7.3 Hz, 3H), 3.09 (dd, J=13.4, 9.8 Hz, 1H), 2.92 (ddd, J=17.1, 13.5, 5.4 Hz, 1H), 2.66-2.54 (m, 1H), 2.11 (d, J=11.5 Hz, 2H), 2.04-1.94 (m, 1H), 1.84 (d, J=12.1 Hz, 2H), 1.69-1.55 (m, 2H), 1.43 (dq, J=36.8, 12.2 Hz, 2H), 1.29 (d, J=6.7 Hz, 3H), 1.20-1.06 (m, 2H), 0.64 (qd, J=7.8, 7.1, 2.2 Hz, 1H), 0.39-0.27 (m, 2H), −0.06 (dd, J=5.3, 3.8 Hz, 2H).

### Compound 603

### 3-{4-[(2-cyclopropylethyl)[(1s,3s)-3-(4H-1,2,4-triazol-3-yl)cyclobutyl]amino]-1-oxo-3H-isoindol-2-yl}piperidine-2,6-dione; formate

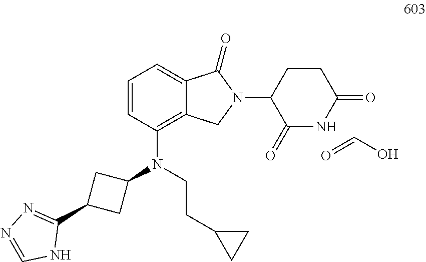

The compound was prepared by the same procedure described as preparation of Compound 573 above.

LC-MS: (ES, m/z): [M+1]=449; ¹H NMR (400 MHz, DMSO-d6) δ 13.70 (s, 1H), 11.04 (s, 1H), 8.23 (s, 1H), 7.46 (t, J=7.7 Hz, 1H), 7.33 (d, J=7.3 Hz, 1H), 7.14 (d, J=7.9 Hz, 1H), 5.18 (dd, J=13.2, 5.1 Hz, 1H), 4.46 (d, J=17.1 Hz, 1H), 4.35 (d, J=17.1 Hz, 1H), 4.09 (h, J=7.6, 7.1 Hz, 1H), 3.32 (d, J=14.8 Hz, 3H), 2.97 (ddd, J=17.0, 13.5, 5.3 Hz, 1H), 2.65 (dt, J=17.5, 3.5 Hz, 3H), 2.55-2.49 (m, 1H), 2.25-2.12 (m, 2H), 2.12-2.01 (m, 1H), 1.25 (ddt, J=23.4, 13.8, 6.8 Hz, 2H), 0.61 (dtd, J=12.1, 7.7, 7.2, 4.9 Hz, 1H), 0.45-0.32 (m, 2H), 0.06 (s, 2H).

### Compound 604

### 3-{4-[(2-cyclopropylethyl)[(1s,3s)-3-(4H-1,2,4-triazol-3-yl)cyclobutyl]amino]-1-oxo-3H-isoindol-2-yl}piperidine-2,6-dione; formate

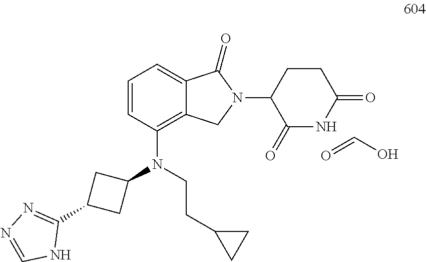

The compound was prepared by the same procedure described as preparation of Compound 573 above.

LC-MS: (ES, m/z): [M+1]=449; ¹H NMR (400 MHz, DMSO-d6) δ 13.76 (s, 1H), 11.06 (s, 1H), 8.62-7.84 (m, 1H), 7.47 (t, J=7.7 Hz, 1H), 7.36 (d, J=7.3 Hz, 1H), 7.07 (d, J=7.8 Hz, 1H), 5.20 (dd, J=13.3, 5.1 Hz, 1H), 4.52-4.33 (m, 3H), 3.32 (d, J=15.0 Hz, 2H), 2.99 (ddd, J=17.2, 13.6, 5.4 Hz, 1H), 2.73-2.59 (m, 1H), 2.55 (d, J=4.5 Hz, 1H), 2.54-2.47 (m, 2H), 2.43-2.33 (m, 2H), 2.17-2.04 (m, 1H), 1.33-1.12 (m, 2H), 0.61 (ddt, J=10.0, 7.4, 3.6 Hz, 1H), 0.45-0.32 (m, 2H), 0.07 (s, 2H).

### Compound 611

### 1-({[(1r,4r)-4-[(2-cyclopropylethyl)[2-(2,6-dioxopiperidin-3-yl)-1-oxo-3H-isoindol-4-yl]amino]cyclohexyl]amino}methyl)cyclopropane-1-carbonitrile

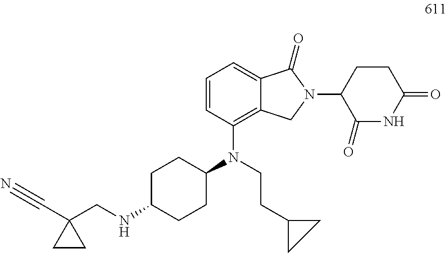 [SMILES: N#CC1(CN[C@H]2CC[C@H](N(CCC3CC3)c3cccc4c3CN(C3CCC(=O)NC3=O)C4=O)CC2)CC1]

The compound was prepared by the general procedure described in the preparation of Compounds 515 and 515A using the appropriate amine.

LCMS: 504.35 (M+H). ¹H NMR (400 MHz, DMSO-d₆): δ 11.03 (s, 1H), 8.93 (d, J=8.1 Hz, 2H), 7.49 (t, J=7.6 Hz, 1H), 7.40-7.34 (m, 2H), 5.21-5.17 (m, 1H), 4.31 (s, 1H), 3.27 (d, J=8.0 Hz, 4H), 3.11 (s, 2H), 3.03-2.95 (m, 1H), 2.83 (d, J=4.9 Hz, 1H), 2.66 (d, J=17.9 Hz, 2H), 2.15 (d, J=11.2 Hz, 2H), 1.91 (s, 2H), 1.62 (d, J=12.4 Hz, 2H), 1.48 (d, J=2.9 Hz, 4H), 1.32-1.27 (m, 3H), 1.20 (dd, J=7.1, 2.7 Hz, 2H), 0.70 (dt, J=7.5, 3.1 Hz, 1H), 0.40-0.37 (m, 2H).

### Compounds 612 and 612A

### 3-{4-[(2-cyclopropylethyl)[(1r,4r)-4-{[(1-fluorocyclopropyl)methyl]amino}cyclohexyl]amino]-1-oxo-3H-isoindol-2-yl}piperidine-2,6-dione and 3-{4-[(2-cyclopropylethyl)[(1s,4s)-4-{[(1-fluorocyclopropyl)methyl]amino}cyclohexyl]amino]-1-oxo-3H-isoindol-2-yl}piperidine-2,6-dione

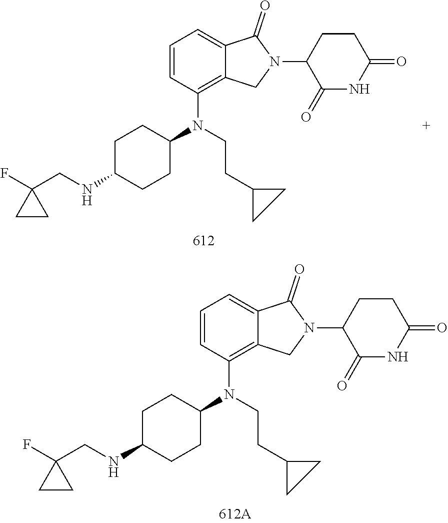 [SMILES: O=C1CCC(N2Cc3c(cccc3N(CCC3CC3)[C@H]3CC[C@H](NCC4(F)CC4)CC3)C2=O)C(=O)N1 ; O=C1CCC(N2Cc3c(cccc3N(CCC3CC3)[C@H]3CC[C@@H](NCC4(F)CC4)CC3)C2=O)C(=O)N1]

The compounds were prepared by the general procedure described in the preparation of Compounds 515 and 515A using the appropriate amine.

Compound 612: LCMS: 497.3 (M+H). ¹H NMR (400 MHz, DMSO-d6) δ 11.03 (s, 1H), 8.98 (d, J=7.6 Hz, 2H), 7.49 (t, J=7.7 Hz, 1H), 7.36 (dd, J=15.1, 7.6 Hz, 2H), 5.18 (dd, J=13.2, 5.2 Hz, 1H), 4.40 (d, J=17.2 Hz, 1H), 4.30 (d, J=17.2 Hz, 1H), 3.53 (t, J=5.9 Hz, 1H), 3.47 (t, J=5.9 Hz, 1H), 3.27 (t, J=7.3 Hz, 2H), 3.15 (s, 2H), 2.98 (ddd, J=17.2, 13.7, 5.4 Hz, 1H), 2.64 (dd, J=16.4, 12.0 Hz, 1H), 2.52 (dd, J=13.2, 4.4 Hz, OH), 2.20 (s, 1H), 2.15 (d, J=15.5 Hz, 1H), 2.11-2.02 (m, 1H), 1.91 (s, 2H), 1.64 (d, J=13.7 Hz, 2H), 1.48 (d, J=11.9 Hz, 2H), 1.27 (d, J=22.0 Hz, 1H), 1.20 (q, J=6.9, 6.0 Hz, 3H), 0.95 (td, J=8.5, 6.4 Hz, 2H), 0.70 (p, J=5.6 Hz, 1H), 0.44-0.33 (m, 2H).

Compound 612A: LCMS: 497.3 (M+H). ¹H NMR (400 MHz, DMSO-d6) δ 11.06 (s, 1H), 8.96 (s, 2H), 7.52 (t, J=7.6 Hz, 1H), 7.43 (dd, J=13.8, 7.5 Hz, 2H), 5.18 (dd, J=13.3, 5.2 Hz, 1H), 4.48 (d, J=17.4 Hz, 1H), 4.37 (d, J=17.3 Hz, 1H), 3.58 (d, J=5.7 Hz, 1H), 3.53 (t, J=5.5 Hz, 1H), 3.44 (s, 1H), 3.33 (s, 1H), 3.27 (q, J=6.6 Hz, 2H), 2.99 (ddd, J=18.0, 13.7, 5.3 Hz, 1H), 2.68 (dd, J=16.9, 3.9 Hz, 1H), 2.61-2.46 (m, 1H), 2.16-2.06 (m, 2H), 1.95 (dd, J=13.2, 7.4 Hz, 4H), 1.79 (s, 2H), 1.60 (d, J=12.1 Hz, 2H), 1.33-1.18 (m, 4H), 1.00 (dd, J=8.8, 6.7 Hz, 2H), 0.70-0.62 (m, 1H), 0.45-0.36 (m, 2H).

### Compounds 613 and 613A

### 3-{4-[(2-cyclopropylethyl)[(1r,4r)-4-{[(1-fluorocyclobutyl)methyl]amino}cyclohexyl]amino]-1-oxo-3H-isoindol-2-yl}piperidine-2,6-dione and 3-{4-[(2-cyclopropylethyl)[(1s,4s)-4-{[(1-fluorocyclobutyl)methyl]amino}cyclohexyl]amino]-1-oxo-3H-isoindol-2-yl}piperidine-2,6-dione

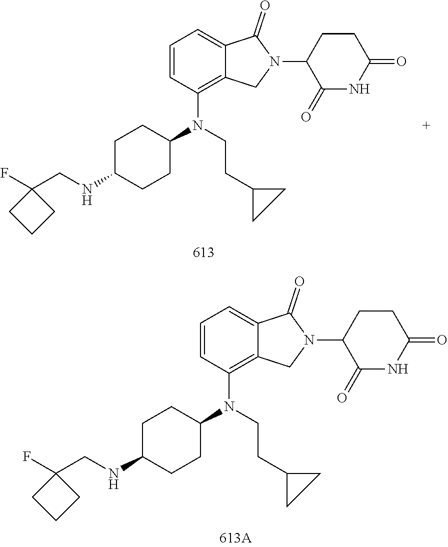 [SMILES: O=C1CCC(N2Cc3c(cccc3N(CCC3CC3)[C@H]3CC[C@H](NCC4(F)CCC4)CC3)C2=O)C(=O)N1 ; O=C1CCC(N2Cc3c(cccc3N(CCC3CC3)[C@H]3CC[C@@H](NCC4(F)CCC4)CC3)C2=O)C(=O)N1]

The compounds were prepared by the general procedure described in the preparation of Compounds 515 and 515A using the appropriate amine.

Compound 613: LCMS: 511.3 (M+H). ¹H NMR (400 MHz, DMSO-d6) δ 11.04 (s, 1H), 8.75 (s, 2H), 7.49 (t, J=7.7 Hz, 1H), 7.37 (dd, J=15.2, 7.6 Hz, 2H), 5.18 (dd, J=13.2, 5.1 Hz, 1H), 4.41 (d, J=17.3 Hz, 1H), 4.30 (d, J=17.2 Hz, 1H), 3.46 (t, J=5.9 Hz, 1H), 3.40 (t, J=6.0 Hz, 1H), 3.27 (t, J=7.3 Hz, 2H), 3.15 (ddt, J=13.2, 9.3, 4.3 Hz, 2H), 2.98 (ddd, J=17.9, 13.5, 5.4 Hz, 1H), 2.70-2.59 (m, 1H), 2.52 (dd, J=13.1, 4.4 Hz, OH), 2.45-2.26 (m, 4H), 2.19 (d, J=11.5 Hz, 2H), 2.06 (ddd, J=11.8, 6.8, 4.5 Hz, 1H), 1.96-1.77 (m, 3H), 1.71-1.58 (m, 3H), 1.50 (dt, J=17.5, 8.9 Hz, 2H), 1.20 (dt, J=9.1, 6.4 Hz, 2H), 0.69 (ddt, J=10.0, 7.3, 3.7 Hz, 1H), 0.44-0.33 (m, 2H).

Compound 613A: LCMS: 511.2 (M+H). ¹H NMR (400 MHz, DMSO-d6) δ 11.06 (s, 1H), 8.96 (s, 2H), 7.52 (t, J=7.6 Hz, 1H), 7.43 (dd, J=13.8, 7.5 Hz, 2H), 5.18 (dd, J=13.3, 5.2 Hz, 1H), 4.48 (d, J=17.4 Hz, 1H), 4.37 (d, J=17.3 Hz, 1H), 3.58 (d, J=5.7 Hz, 1H), 3.53 (t, J=5.5 Hz, 1H), 3.44 (s, 1H), 3.33 (s, 1H), 3.27 (q, J=6.6 Hz, 2H), 2.99 (ddd, J=18.0, 13.7, 5.3 Hz, 1H), 2.68 (dd, J=16.9, 3.9 Hz, 1H), 2.61-2.46 (m, 1H), 2.16-2.06 (m, 2H), 1.95 (dd, J=13.2, 7.4 Hz, 4H), 1.79 (s, 2H), 1.60 (d, J=12.1 Hz, 2H), 1.33-1.18 (m, 4H), 1.00 (dd, J=8.8, 6.7 Hz, 2H), 0.70-0.62 (m, 1H), 0.45-0.36 (m, 2H).

### Compounds 614 and 614A

### 3-{4-[(2-cyclopropylethyl)[(1r,4r)-4-({[1-(trifluoromethyl)cyclobutyl]methyl}amino)cyclohexyl]amino]-1-oxo-3H-isoindol-2-yl}piperidine-2,6-dione; trifluoro acetate and 3-{4-[(2-cyclopropylethyl)[(1s,4s)-4-({[1-(trifluoromethyl)cyclobutyl]methyl}amino)cyclohexyl]amino]-1-oxo-3H-isoindol-2-yl}piperidine-2,6-dione; trifluoro acetate

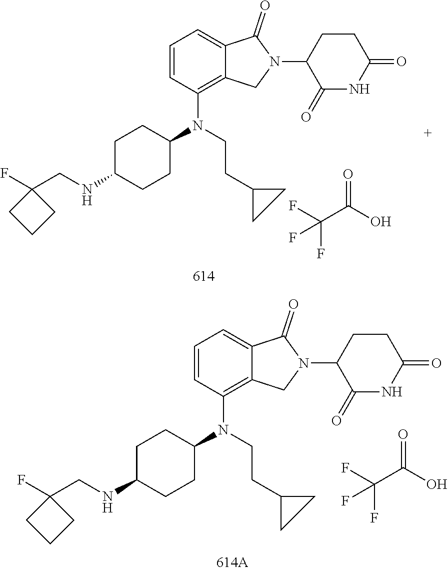

The compounds were prepared by the general procedure described in the preparation of Compounds 515 and 515A using the appropriate amine.

Compound 614: LCMS: 561.3 (M+H). ¹H NMR (400 MHz, DMSO-d6): δ 11.02 (s, 1H), 8.61 (s, 2H), 7.49 (t, J=7.6 Hz, 1H), 7.36 (dd, J=13.9, 7.6 Hz, 2H), 5.18 (dd, J=13.2, 5.1 Hz, 1H), 4.40 (d, J=17.2 Hz, 1H), 4.30 (d, J=17.2 Hz, 1H), 3.40 (t, J=6.0 Hz, 2H), 3.27 (t, J=7.1 Hz, 2H), 3.16 (s, 2H), 2.97 (ddd, J=17.8, 13.5, 5.4 Hz, 1H), 2.67 (d, J=3.5 Hz, 1H), 2.65-2.59 (m, 1H), 2.30 (ddd, J=12.8, 9.9, 6.5 Hz, 2H), 2.25-2.14 (m, 4H), 2.13-2.00 (m, 2H), 1.99-1.86 (m, 3H), 1.72-1.47 (m, 4H), 1.27-1.12 (m, 2H), 0.69 (td, J=7.6, 3.9 Hz, 1H), 0.39 (dt, J=8.4, 2.8 Hz, 2H), −0.01 (q, J=4.8 Hz, 2H).

Compound 614A: LCMS: 561.3 (M+H). ¹H NMR (400 MHz, DMSO-d6): δ 11.08 (s, 1H), 8.61 (s, 2H), 7.61-7.37 (m, 3H), 5.21 (dd, J=13.3, 5.2 Hz, 2H), 4.57-4.34 (m, 2H), 3.62-3.39 (m, 3H), 3.25 (hept, J=6.8 Hz, 3H), 3.01 (ddd, J=17.2, 13.6, 5.4 Hz, 1H), 2.68 (dd, J=17.2, 3.7 Hz, 1H), 2.52 (dd, J=13.2, 4.5 Hz, 1H), 2.34 (ddd, J=12.9, 9.7, 6.6 Hz, 2H), 2.28-2.17 (m, 2H), 2.17-2.05 (m, 2H), 2.05-1.79 (m, 7H), 1.59 (d, J=11.9 Hz, 2H), 1.34-1.17 (m, 2H), 0.64 (ddt, J=10.2, 7.3, 3.6 Hz, 1H), 0.47-0.34 (m, 2H), −0.00 (td, J=5.0, 3.3 Hz, 2H).

### Compounds 615 and 615A

### 3-{4-[(2-cyclopropylethyl)[(1r,4r)-4-{[(1-methanesulfonylcyclopropyl)methyl]amino}cyclohexyl]amino]-1-oxo-3H-isoindol-2-yl}piperidine-2,6-dione; trifluoroacetate and 3-{4-[(2-cyclopropylethyl)[(1s,4s)-4-{[(1-methanesulfonylcyclopropyl)methyl]amino}cyclohexyl]amino]-1-oxo-3H-isoindol-2-yl}piperidine-2,6-dione; formate

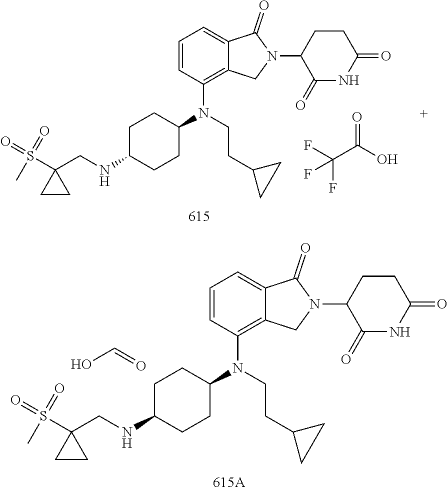

The compounds were prepared by the general procedure described in the preparation of Compounds 515 and 515A using the appropriate amine.

Compound 615: LCMS: 557.3 (M+H). ¹H NMR (400 MHz, DMSO-d6): δ 11.03 (s, 1H), 8.55 (s, 2H), 7.49 (t, J=7.6 Hz, 1H), 7.36 (dd, J=15.6, 7.6 Hz, 2H), 5.18 (dd, J=13.2, 5.1 Hz, 1H), 4.47-4.23 (m, 2H), 3.57 (t, J=5.6 Hz, 2H), 3.27 (d, J=7.4 Hz, 2H), 3.24 (s, 3H), 3.12 (d, J=13.2 Hz, 2H), 2.98 (ddd, J=17.8, 13.5, 5.4 Hz, 1H), 2.70-2.60 (m, 1H), 2.21-2.10 (m, 2H), 2.06 (dd, J=9.4, 4.0 Hz, 1H), 1.90 (s, 2H), 1.72-1.42 (m, 7H), 1.37-1.27 (m, 3H), 1.25-1.13 (m, 2H), 0.69 (ddt, J=9.8, 7.1, 3.4 Hz, 1H), 0.43-0.33 (m, 2H), −0.00 (dd, J=4.9, 1.5 Hz, 2H).

Compound 615A: LCMS: 557.4 (M+H). ¹H NMR (400 MHz, DMSO-d6): δ 11.03 (s, 1H), 8.29 (s, 1H), 7.46 (td, J=7.7, 2.5 Hz, 1H), 7.31 (ddd, J=16.4, 7.7, 2.5 Hz, 2H), 5.16 (ddd, J=13.2, 5.3, 2.4 Hz, 1H), 4.49-4.26 (m, 2H), 3.37-3.25 (m, 2H), 3.23 (d, J=2.5 Hz, 4H), 3.03-2.87 (m, 3H), 2.73-2.59 (m, 2H), 2.55-2.47 (m, 1H), 2.08 (dh, J=13.9, 4.1, 3.3 Hz, 1H), 2.00-1.70 (m, 4H), 1.59-1.40 (m, 4H), 1.34-1.17 (m, 4H), 1.07 (q, J=4.4 Hz, 2H), 0.69 (ddd, J=12.4, 8.6, 4.8 Hz, 1H), 0.40 (qd, J=6.0, 3.3 Hz, 2H), −0.00 (dt, J=4.9, 2.5 Hz, 2H).

### Compounds 616 and 616A

### 3-{4-[(2-cyclopropylethyl)[(1r,4r)-4-{[(3-fluorooxetan-3-yl)methyl]amino}cyclohexyl]amino]-1-oxo-3H-isoindol-2-yl}piperidine-2,6-dione; trifluoro acetate and 3-{4-[(2-cyclopropylethyl)[(1s,4s)-4-{[(3-fluorooxetan-3-yl)methyl]amino}cyclohexyl]amino]-1-oxo-3H-isoindol-2-yl}piperidine-2,6-dione; trifluoroacetate

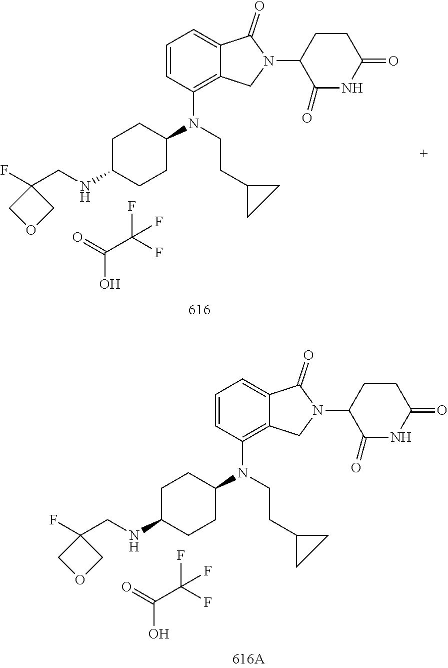

The compounds were prepared by the general procedure described in the preparation of Compounds 515 and 515A using the appropriate amine.

Compound 616: LC-MS: (ES, m/z): [M+1]=513.2; ¹H NMR (400 MHz, DMSO-d6) δ 11.04 (s, 1H), 8.95 (s, 2H), 7.50 (t, J=7.6 Hz, 1H), 7.37 (dd, J=15.6, 7.6 Hz, 2H), 5.18 (dd, J=13.2, 5.1 Hz, 1H), 4.80-4.66 (m, 4H), 4.48-4.21 (m, 2H), 3.66 (d, J=4.9 Hz, 2H), 3.27 (t, J=7.4 Hz, 2H), 3.16 (q, J=11.6, 8.0 Hz, 2H), 2.97 (ddd, J=17.9, 13.5, 5.4 Hz, 1H), 2.72-2.58 (m, 2H), 2.53 (dd, J=13.2, 4.5 Hz, 1H), 2.17 (d, J=11.5 Hz, 2H), 2.07 (dt, J=11.9, 5.7 Hz, 1H), 1.92 (dd, J=12.3, 7.4 Hz, 2H), 1.69-1.57 (m, 2H), 1.50 (t, J=13.0 Hz, 2H), 1.20 (q, J=7.3 Hz, 2H), 0.69 (tq, J=12.2, 7.0, 6.3 Hz, 1H), 0.44-0.35 (m, 2H), −0.00 (d, J=43.5 Hz, 2H). ¹⁹F NMR (377 MHz, DMSO-d6) δ −70.74-−76.29 (m).

Compound 616A: LC-MS: (ES, m/z): [M+1]=513.2; ¹H NMR (400 MHz, DMSO-d6) δ 11.07 (s, 1H), 8.97 (d, J=7.7 Hz, 2H), 7.52 (t, J=7.5 Hz, 1H), 7.45 (dd, J=14.2, 7.5 Hz, 2H), 5.19 (dd, J=13.3, 5.1 Hz, 1H), 4.76 (dt, J=22.1, 6.8 Hz, 4H), 4.49 (d, J=17.4 Hz, 1H), 4.38 (d, J=17.3 Hz, 1H), 3.79 (d, J=5.2 Hz, 1H), 3.72 (d, J=5.3 Hz, 1H), 3.47 (dq, J=6.7, 3.4 Hz, 1H), 3.34-3.19 (m, J=6.9 Hz, 3H), 3.00 (ddd, J=18.1, 13.5, 5.4 Hz, 1H), 2.73-2.64 (m, 1H), 2.52 (td, J=13.1, 4.4 Hz, 2H), 2.11 (dt, J=11.9, 5.8 Hz, 1H), 1.93 (td, J=11.5, 5.6 Hz, 4H), 1.79 (tt, J=9.5, 4.1 Hz, 2H), 1.59 (dq, J=15.9, 5.9 Hz, 2H), 1.26 (dp, J=14.4, 7.3, 6.8 Hz, 2H), 0.66 (hept, J=6.7 Hz, 1H), 0.40 (d, J=7.8 Hz, 2H). ¹⁹F NMR (377 MHz, DMSO-d6) δ −71.42-−76.82 (m).

### Compound 619

### 3-(4-((2-cyclopropylethyl)(4-(((1-(trifluoromethyl)cyclopropyl)methyl)amino)cyclohexyl)amino)-5-fluoro-1-oxoisoindolin-2-yl)piperidine-2,6-dione

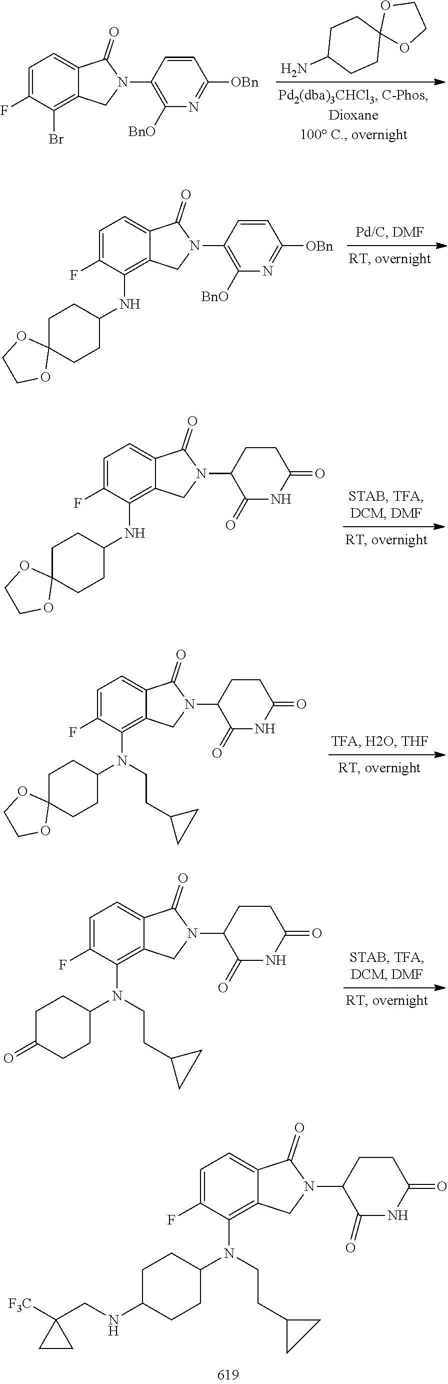

### Step-1:2-[2,6-bis(benzyloxy)pyridin-3-yl]-4-(1,4-dioxaspiro[4.5]decan-8-ylamino-5-fluoro-3H-isoindol-1-one

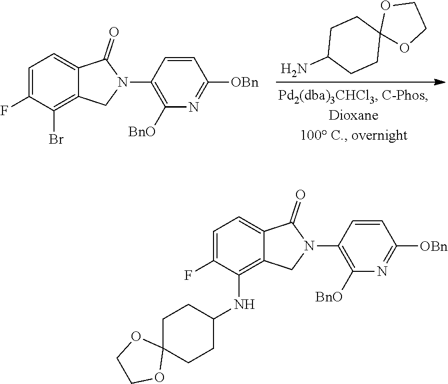 [SMILES: O=C1c2ccc(F)c(Br)c2CN1c1ccc(OCc2ccccc2)nc1OCc1ccccc1 ; NC1CCC2(CC1)OCCO2 ; O=C1c2ccc(F)c(NC3CCC4(CC3)OCCO4)c2CN1c1ccc(OCc2ccccc2)nc1OCc1ccccc1]

A solution of 2-[2,6-bis(benzyloxy)pyridin-3-yl]-4-bromo-5-fluoro-3H-isoindol-1-one (1 g, 1.9 mmol, 1 equiv) in dioxane (10 mL) was treated with 1,4-dioxaspiro[4.5]decan-8-amine (605 mg, 3.85 mmol, 2 equiv) and Cs₂CO₃ (2509 mg, 7.7 mmol, 4 equiv) for 5 min at room temperature under nitrogen atmosphere. To the above mixture was added CPhos (168 mg, 0.38 mmol, 0.2 equiv) and Pd₂(dba)₃CHCl₃ (199 mg, 0.19 mmol, 0.1 equiv) at room temperature. The resulting mixture was stirred for additional overnight at 100° C. The resulting mixture was diluted with water (50 mL). The resulting mixture was extracted with EtOAc (3×50 mL). The combined organic layers were washed with brine (3×100 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 2-[2,6-bis(benzyloxy)pyridin-3-yl]-4-(1,4-dioxaspiro[4.5]decan-8-ylamino-5-fluoro-3H-isoindol-1-one (700 mg, 61%) as a yellow solid.

### Step-2: 3-(4-(1,4-dioxaspiro[4.5]decan-8-ylamino-5-fluoro-1-oxo-3H-isoindol-2-yl)piperidine-2,6-dione

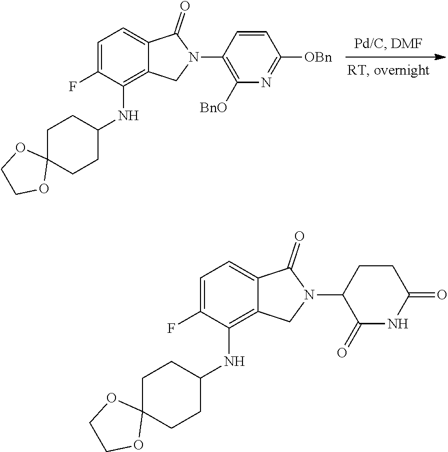 [SMILES: O=C1c2ccc(F)c(NC3CCC4(CC3)OCCO4)c2CN1c1ccc(OCc2ccccc2)nc1OCc1ccccc1 ; O=C1CCC(N2Cc3c(ccc(F)c3NC3CCC4(CC3)OCCO4)C2=O)C(=O)N1]

To a solution of 2-[2,6-bis(benzyloxy)pyridin-3-yl]-4-(1,4-dioxaspiro[4.5]decan-8-ylamino-5-fluoro-3H-isoindol-1-one (550 mg, 0.92 mmol, 1 equiv) in DMF (5 mL) was added Palladium 10% on Carbon (wetted with ca. 55% Water) (550 mg, 0.52 mmol, 0.56 equiv, 10%) at room temperature. The resulting mixture was stirred overnight at room temperature under hydrogen atmosphere. The resulting mixture was filtered, the filter cake was washed with EtOAc (5×10 mL). The filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 3-(4-(1,4-dioxaspiro[4.5]decan-8-ylamino-5-fluoro-1-oxo-3H-isoindol-2-yl)piperidine-2,6-dione (170 mg, 44%) as a purple solid.

### Step-3: 3-(4-[(2-cyclopropylethyl)(1,4-dioxaspiro[4.5]decan-8-yl)amino]-5-fluoro-1-oxo-3H-isoindol-2-ylpiperidine-2,6-dione

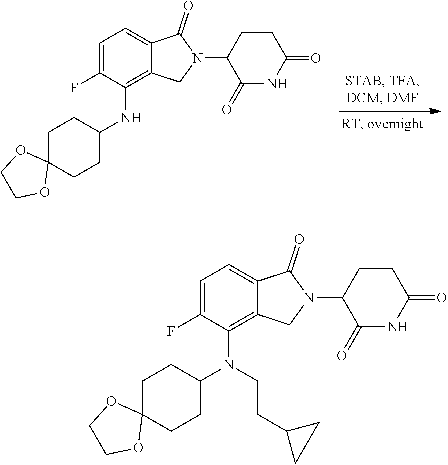 [SMILES: O=C1CCC(N2Cc3c(ccc(F)c3NC3CCC4(CC3)OCCO4)C2=O)C(=O)N1 ; O=C1CCC(N2Cc3c(ccc(F)c3N(CCC3CC3)C3CCC4(CC3)OCCO4)C2=O)C(=O)N1]

A solution of 3-(4-(1,4-dioxaspiro[4.5]decan-8-ylamino-5-fluoro-1-oxo-3H-isoindol-2-yl)piperidine-2,6-dione (160 mg, 0.38 mmol, 1 equiv) in DCM (4 mL) and DMF (1 ml) was treated with 2-cyclopropylacetaldehyde (644 mg, 7.6 mmol, 20 equiv) under nitrogen atmosphere. To the above mixture was added TFA (131 mg, 1.15 mmol, 3 equiv) dropwise at room temperature. The resulting mixture was stirred for additional overnight at room temperature. To the above mixture was added STAB (406 mg, 1.9 mmol, 5 equiv) in portions at 0° C. The resulting mixture was diluted with water (20 mL). The resulting mixture was extracted with EtOAc (3×20 mL). The combined organic layers were washed with water (3×100 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 3-(4-[(2-cyclopropylethyl)(1,4-dioxaspiro[4.5]decan-8-yl)amino]-5-fluoro-1-oxo-3H-isoindol-2-ylpiperidine-2,6-dione) (150 mg, 81%) as a white solid.

### Step-4: 3-(4-[(2-cyclopropylethyl)(4-oxocyclohexyl)amino]-5-fluoro-1-oxo-3H-isoindol-2-ylpiperidine-2,6-dione

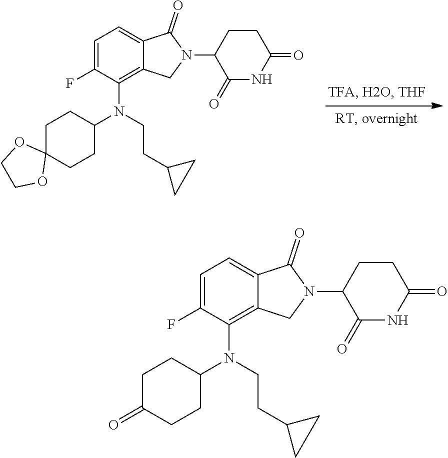 [SMILES: O=C1CCC(N2Cc3c(ccc(F)c3N(CCC3CC3)C3CCC4(CC3)OCCO4)C2=O)C(=O)N1 ; O=C1CCC(N(CCC2CC2)c2c(F)ccc3c2CN(C2CCC(=O)NC2=O)C3=O)CC1]

To a solution of 3-(4-[(2-cyclopropylethyl)(1,4-dioxaspiro[4.5]decan-8-yl)amino]-5-fluoro-1-oxo-3H-isoindol-2-ylpiperidine-2,6-dione (133 mg, 0.274 mmol, 1 equiv) in THF (1.3 mL) and H₂O (1.3 mL) was added TFA (0.13 mL, 1.79 mmol, 6.5 equiv) dropwise at room temperature. The resulting mixture was stirred for additional overnight at room temperature. The resulting mixture was concentrated under reduced pressure. The residue was dissolved in water (25 mL). The resulting mixture was extracted with EtOAc (3×20 mL). The combined organic layers were washed with brine (3×50 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 3-(4-[(2-cyclopropylethyl)(4-oxocyclohexyl)amino]-5-fluoro-1-oxo-3H-isoindol-2-ylpiperidine-2,6-dione (85 mg, 70%) as a yellow solid.

### Step-5: 3-(4-((2-cyclopropylethyl)(4-(((1-(trifluoromethyl)cyclopropyl)methyl)amino)cyclohexyl)amino)-5-fluoro-1-oxoisoindolin-2-yl)piperidine-2,6-dione

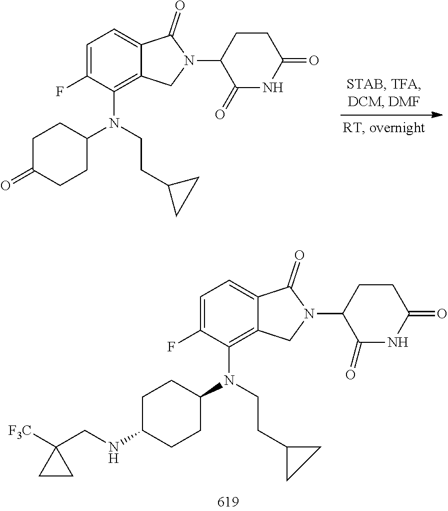 [SMILES: O=C1CCC(N(CCC2CC2)c2c(F)ccc3c2CN(C2CCC(=O)NC2=O)C3=O)CC1 ; O=C1CCC(N2Cc3c(ccc(F)c3N(CCC3CC3)[C@H]3CC[C@H](NCC4(C(F)(F)F)CC4)CC3)C2=O)C(=O)N1]

A solution of 3-(4-[(2-cyclopropylethyl)(4-oxocyclohexyl)amino]-5-fluoro-1-oxo-3H-isoindol-2-ylpiperidine-2,6-dione (65 mg, 0.147 mmol, 1 equiv) in DCM (1.6 mL) and DMF (0.4 mL) was treated with 1-[1-(trifluoromethyl)cyclopropyl]methanamine (102 mg, 0.73 mmol, 5 equiv) for 10 min under nitrogen atmosphere. To the above mixture was added TFA (50 mg, 0.44 mmol, 3 equiv) dropwise at room temperature. The resulting mixture was stirred for additional overnight at room temperature. To the above mixture was added STAB (156 mg, 0.74 mmol, 5 equiv) in portions at 0° C. The resulting mixture was diluted with water (20 mL). The resulting mixture was extracted with EtOAc (3×20 mL). The combined organic layers were washed with water (3×50 mL), dried over anhydrous Na₂SO₄. After filtration, the filtrate was concentrated under reduced pressure. The residue was purified by Prep-HPLC with the following conditions: Column: Sunfire prep C18 column, 30*150 mm, 5 m; Mobile Phase A: Water (0.1% FA), Mobile Phase B: 20 mm NaOH+10% ACN; Flow rate: 60 mL/min mL/min; Gradient: isocratic 5% B to 25% B in 10 min; Wave Length: 254 nm/220 nm; RT1 (min): 12.55. This resulted in 3-(4-((2-cyclopropylethyl)(4-(((1-(trifluoromethyl)cyclopropyl)methyl)amino)cyclohexyl)amino)-5-fluoro-1-oxoisoindolin-2-yl)piperidine-2,6-dione (35 mg, 42%) as a white solid.

LCMS: 565.35 (M+H); ¹H NMR (400 MHz, DMSO-d₆) δ 10.97 (s, 1H), 7.48 (ddd, J=7.3, 4.3, 2.9 Hz, 1H), 7.35-7.27 (m, 1H), 5.12-5.05 (m, 1H), 4.31 (qd, J=17.6, 6.6 Hz, 2H), 3.14 (t, J=7.3 Hz, 2H), 3.07-2.81 (m, 2H), 2.73 (d, J=14.6 Hz, 2H), 2.58 (d, J=16.9 Hz, 1H), 2.45 (dd, J=13.1, 4.4 Hz, 1H), 2.37-2.19 (m, 1H), 2.07-1.93 (m, 1H), 1.89-1.77 (m, 2H), 1.72 (d, J=17.1 Hz, 2H), 1.60 (s, 1H), 1.39 (dd, J=29.6, 15.4 Hz, 3H), 1.11 (dq, J=12.7, 6.7, 5.4 Hz, 2H), 0.98 (d, J=12.7 Hz, 1H), 0.88-0.68 (m, 4H), 0.61 (dt, J=12.7, 6.8 Hz, 1H), 0.31 (ddt, J=8.4, 5.5, 2.8 Hz, 2H), −0.09 (tt, J=5.5, 2.8 Hz, 2H).

### Compounds 627 and 627A

### 3-{4-[(2-cyclopropylethyl)[(1r,4r)-4-({[1-(trifluoromethyl)cyclopropyl]methyl}amino)cyclohexyl]amino]-7-fluoro-1-oxo-3H-isoindol-2-yl}piperidine-2,6-dione and 3-{4-[(2-cyclopropylethyl)[(1s,4s)-4-({[1-(trifluoromethyl)cyclopropyl]methyl}amino)cyclohexyl]amino]-7-fluoro-1-oxo-3H-isoindol-2-yl}piperidine-2,6-dione

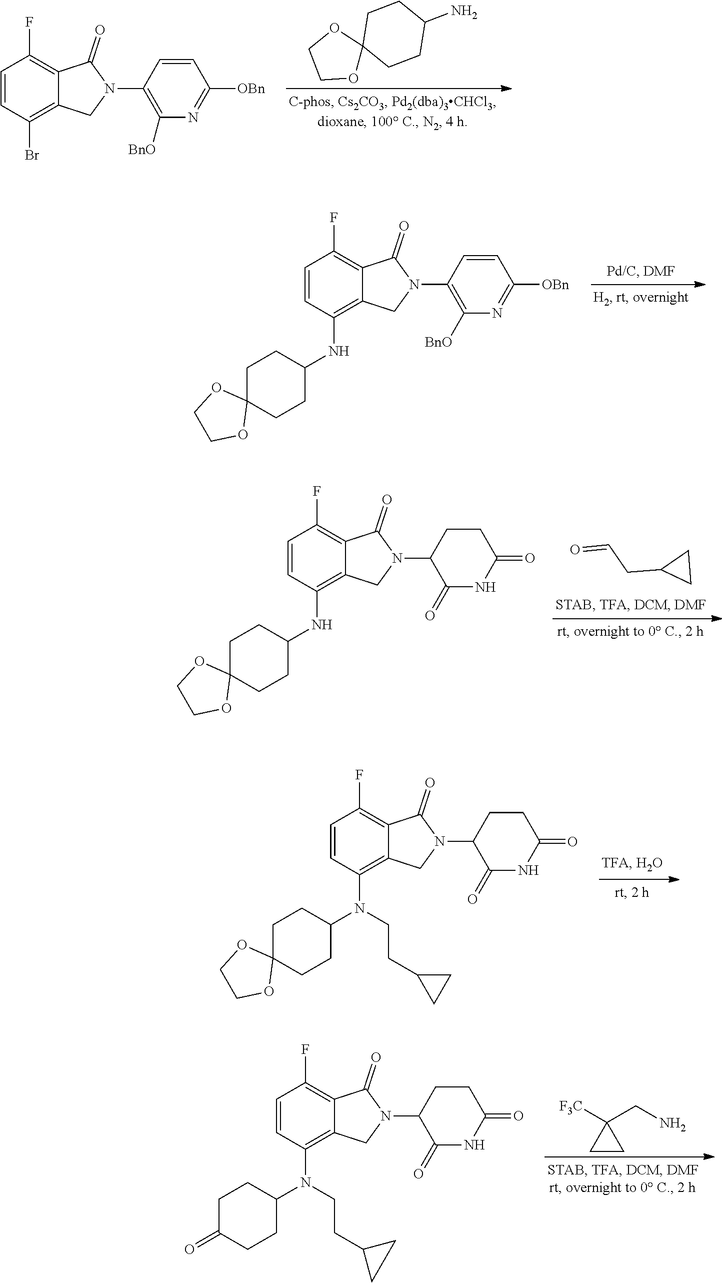

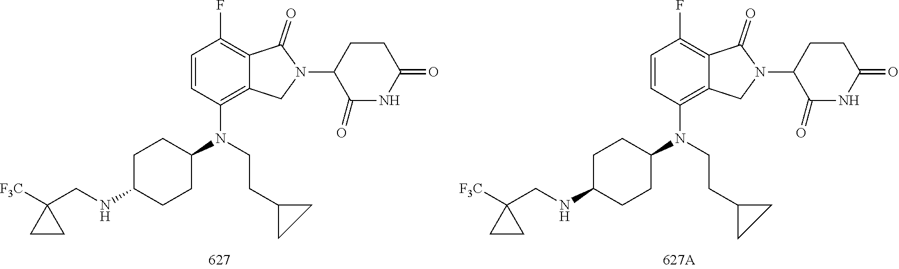 [SMILES: O=C1CCC(N2Cc3c(N(CCC4CC4)[C@H]4CC[C@H](NCC5(C(F)(F)F)CC5)CC4)ccc(F)c3C2=O)C(=O)N1 ; O=C1CCC(N2Cc3c(N(CCC4CC4)[C@H]4CC[C@@H](NCC5(C(F)(F)F)CC5)CC4)ccc(F)c3C2=O)C(=O)N1]

### Step-1:2-[2,6-bis(benzyloxy)pyridin-3-yl]-4-{1,4-dioxaspiro[4.5]decan-8-ylamino}-7-fluoro-3H-isoindol-1-one

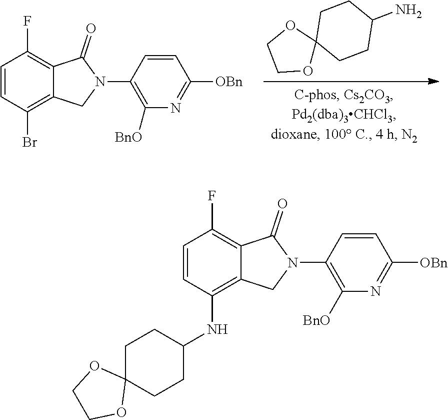 [SMILES: O=C1c2c(F)ccc(Br)c2CN1c1ccc(OCc2ccccc2)nc1OCc1ccccc1 ; NC1CCC2(CC1)OCCO2 ; O=C1c2c(F)ccc(NC3CCC4(CC3)OCCO4)c2CN1c1ccc(OCc2ccccc2)nc1OCc1ccccc1]

To a stirred mixture of 2-[2,6-bis(benzyloxy)pyridin-3-yl]-4-bromo-7-fluoro-3H-isoindol-1-one (1140 mg, 2.19 mmol, 1 equiv) and 1,4-dioxaspiro[4.5]decan-8-amine (517 mg, 3.29 mmol, 1.5 equiv) in 1,4-dioxane were added Cs₂CO₃ (2860 mg, 8.8 mmol, 4 equiv) and CPhos (192 mg, 0.44 mmol, 0.2 equiv) at room temperature under nitrogen atmosphere. To the above mixture was added Pd₂(dba)₃·CHCl₃ (227 mg, 0.22 mmol, 0.1 equiv). The resulting mixture was stirred for additional overnight at 100° C. The mixture was allowed to cool down to room temperature. The resulting mixture was diluted with water (20 mL). The resulting mixture was extracted with EtOAc (3×10 mL). The combined organic layers were washed with water (2×12 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 2-[2,6-bis(benzyloxy)pyridin-3-yl]-4-{1,4-dioxaspiro[4.5]decan-8-ylamino}-7-fluoro-3H-isoindol-1-one (377 mg, 28%) as a brown solid.

### Step-2: 3-(4-{1,4-dioxaspiro[4.5]decan-8-ylamino}-7-fluoro-1-oxo-3H-isoindol-2-yl)piperidine-2,6-dione

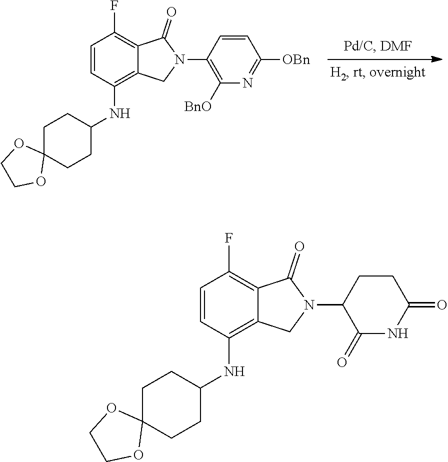 [SMILES: O=C1c2c(F)ccc(NC3CCC4(CC3)OCCO4)c2CN1c1ccc(OCc2ccccc2)nc1OCc1ccccc1 ; O=C1CCC(N2Cc3c(NC4CCC5(CC4)OCCO5)ccc(F)c3C2=O)C(=O)N1]

A mixture of 2-[2,6-bis(benzyloxy)pyridin-3-yl]-4-{1,4-dioxaspiro[4.5]decan-8-ylamino}-7-fluoro-3H-isoindol-1-one (377 mg, 0.63 mmol, 1 equiv) and Palladium 10% on Carbon (wetted with ca. 55% Water) (377 mg, 3.54 mmol, 5.6 equiv) in DMF (5 ml) was stirred for overnight at room temperature under hydrogen atmosphere. The resulting mixture was filtered, the filter cake was washed with THF (3×5 mL). The filtrate was concentrated under reduced pressure. The residue was purified by reversed-phase flash chromatography with the following conditions: column, C18 silica gel; mobile phase, MeCN in Water (0.1% FA), 10% to 100% gradient in 20 min; detector, UV 254 nm. This resulted in 3-(4-{1,4-dioxaspiro[4.5]decan-8-ylamino}-7-fluoro-1-oxo-3H-isoindol-2-yl)piperidine-2,6-dione (160 mg, 56%) as a light yellow solid.

### Step-3: 3-{4-[(2-cyclopropylethyl)(1,4-dioxaspiro[4.5]decan-8-yl)amino]-7-fluoro-1-oxo-3H-isoindol-2-yl}piperidine-2,6-dione

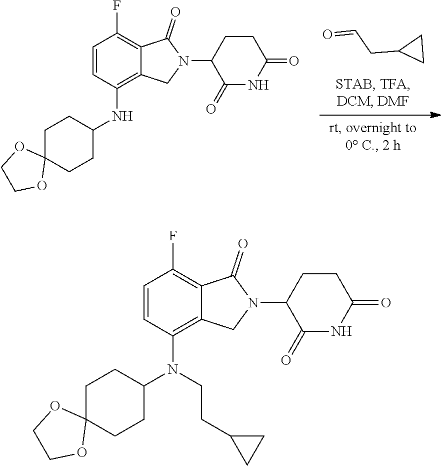 [SMILES: O=C1CCC(N2Cc3c(NC4CCC5(CC4)OCCO5)ccc(F)c3C2=O)C(=O)N1 ; O=CCC1CC1 ; O=C1CCC(N2Cc3c(N(CCC4CC4)C4CCC5(CC4)OCCO5)ccc(F)c3C2=O)C(=O)N1]

To a stirred mixture of 3-(4-{1,4-dioxaspiro[4.5]decan-8-ylamino}-7-fluoro-1-oxo-3H-isoindol-2-yl)piperidine-2,6-dione (145 mg, 0.347 mmol, 1 equiv) and 2-cyclopropylacetaldehyde (584 mg, 6.9 mmol, 20 equiv) in DCM (3.6 ml) and DMF (0.9 ml) was added TFA (118 mg, 1.04 mmol, 3 equiv) dropwise at room temperature under nitrogen atmosphere. The resulting mixture was stirred for additional overnight at room temperature. To the above mixture was added STAB (368 mg, 1.73 mmol, 5 equiv) at 0° C. The resulting mixture was stirred for additional 2 h at room temperature. The resulting mixture was diluted with water (10 mL). The resulting mixture was extracted with EtOAc (1×10 mL). The combined organic layers were washed with water (3×15 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 3-{4-[(2-cyclopropylethyl)(1,4-dioxaspiro[4.5]decan-8-yl)amino]-7-fluoro-1-oxo-3H-isoindol-2-yl}piperidine-2,6-dione (100 mg, 58%) as a light yellow solid.

### Step-4:3-{4-[(2-cyclopropylethyl)(4-oxocyclohexyl)amino]-7-fluoro-1-oxo-3H-isoindol-2-yl}piperidine-2,6-dione

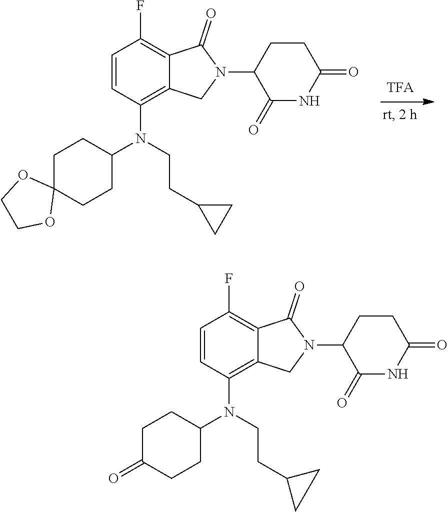

A solution of 3-{4-[(2-cyclopropylethyl)(1,4-dioxaspiro[4.5]decan-8-yl)amino]-7-fluoro-1-oxo-3H-isoindol-2-yl}piperidine-2,6-dione (100 mg, 0.21 mmol, 1 equiv) and TFA (1 mL, 13.4 mmol, 65 equiv) in THF (1 ml) and H₂O (1 ml) was stirred for 3 h at room temperature under nitrogen atmosphere. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 3-{4-[(2-cyclopropylethyl)(4-oxocyclohexyl)amino]-7-fluoro-1-oxo-3H-isoindol-2-yl}piperidine-2,6-dione (86 mg, 86%) as a yellow green solid.

### Step-5: 3-{4-[(2-cyclopropylethyl)[(1r,4r)-4-({[1-(trifluoromethyl)cyclopropyl]methyl}amino)cyclohexyl]amino]-7-fluoro-1-oxo-3H-isoindol-2-yl}piperidine-2,6-dione and 3-{4-[(2-cyclopropylethyl)[(1s,4s)-4-({[1-(trifluoromethyl)cyclopropyl]methyl}amino)cyclohexyl]amino]-7-fluoro-1-oxo-3H-isoindol-2-yl}piperidine-2,6-dione

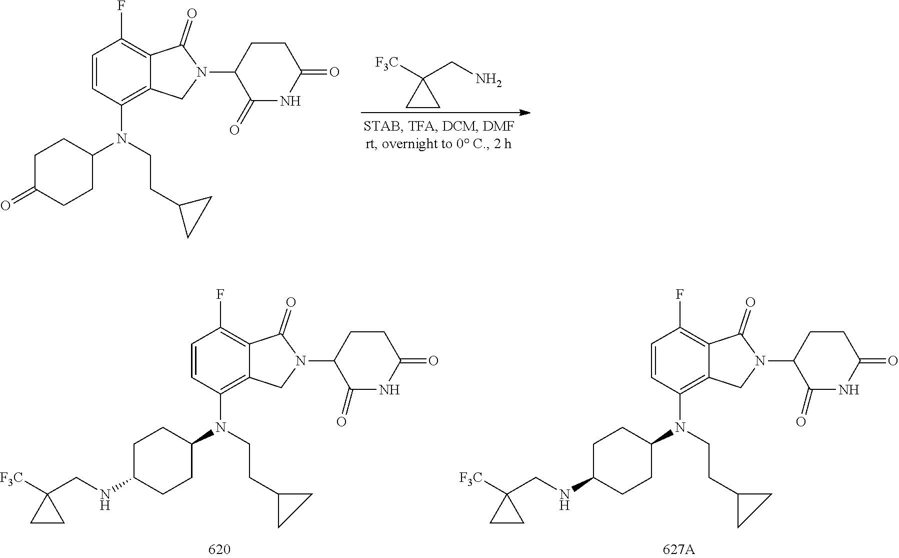 [SMILES: O=C1CCC(N(CCC2CC2)c2ccc(F)c3c2CN(C2CCC(=O)NC2=O)C3=O)CC1 ; NCC1(C(F)(F)F)CC1 ; O=C1CCC(N2Cc3c(N(CCC4CC4)[C@H]4CC[C@H](NCC5(C(F)(F)F)CC5)CC4)ccc(F)c3C2=O)C(=O)N1 ; O=C1CCC(N2Cc3c(N(CCC4CC4)[C@H]4CC[C@@H](NCC5(C(F)(F)F)CC5)CC4)ccc(F)c3C2=O)C(=O)N1]

To a stirred mixture of 3-{4-[(2-cyclopropylethyl)(4-oxocyclohexyl)amino]-7-fluoro-1-oxo-3H-isoindol-2-yl}piperidine-2,6-dione (70 mg, 0.16 mmol, 1 equiv) and 1-[1-(trifluoromethyl)cyclopropyl]methanamine (110 mg, 0.79 mmol, 5 equiv) in DCM and DMF was added TFA (54 mg, 0.48 mmol, 3 equiv) dropwise at 0° C. under nitrogen atmosphere. The mixture was stirred for additional overnight at room temperature. To the above mixture was added STAB (168 mg, 0.79 mmol, 5 equiv) at 0° C. The resulting mixture was stirred for additional 2 h at room temperature. The resulting mixture was diluted with water (10 mL). The resulting mixture was extracted with EtOAc (3×5 mL). The combined organic layers were washed with water (3×6 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 3-{4-[(2-cyclopropylethyl)[(1r,4r)-4-({[1-(trifluoromethyl)cyclopropyl]methyl}amino)cyclohexyl]amino]-7-fluoro-1-oxo-3H-isoindol-2-yl}piperidine-2,6-dione (29.3 mg, 32%) as an off-white solid and 3-{4-[(2-cyclopropylethyl)[(1s,4s)-4-({[1-(trifluoromethyl)cyclopropyl]methyl}amino)cyclohexyl]amino]-7-fluoro-1-oxo-3H-isoindol-2-yl}piperidine-2,6-dione (29 mg, 31%) as an off-white solid.

Compound 627: LCMS: 565.3 (M+H). ¹H NMR (400 MHz, DMSO-d6): δ 10.98 (s, 1H), 7.38 (dd, J=8.8, 3.9 Hz, 1H), 7.23 (d, J=8.9 Hz, 1H), 5.08 (dd, J=13.2, 5.1 Hz, 2H), 4.31 (d, J=17.9 Hz, 2H), 4.22 (d, J=17.8 Hz, 2H), 3.24 (t, J=6.0 Hz, 3H), 3.00 (s, 1H), 2.90 (dq, J=14.1, 7.2, 6.1 Hz, 3H), 2.58 (dd, J=19.0, 4.7 Hz, 2H), 2.06 (d, J=10.4 Hz, 3H), 1.85 (s, 2H), 1.42 (q, J=14.9, 14.4 Hz, 6H), 1.24 (s, 1H), 0.61 (d, J=5.7 Hz, 1H), 0.32 (dt, J=8.5, 2.8 Hz, 3H), −0.08 (q, J=4.8 Hz, 3H).

Compound 627A: LCMS: 565.3 (M+H). ¹H NMR (400 MHz, DMSO-d6): δ 11.09 (s, 1H), 7.51 (dd, J=8.8, 4.0 Hz, 1H), 7.31 (t, J=8.7 Hz, 1H), 5.17 (dd, J=13.3, 5.2 Hz, 1H), 4.49 (d, J=17.8 Hz, 1H), 4.38 (d, J=17.8 Hz, 1H), 3.35 (d, J=11.8 Hz, 3H), 3.18 (dq, J=19.7, 6.8, 6.0 Hz, 3H), 3.00 (ddd, J=18.0, 13.5, 5.4 Hz, 1H), 2.68 (dd, J=17.3, 3.7 Hz, 1H), 2.58-2.43 (m, 4H), 2.15-2.07 (m, 1H), 1.89-1.84 (m, 2H), 1.78 (s, 2H), 1.54 (s, 2H), 1.25-1.10 (m, 5H), 0.69-0.61 (m, 1H), 0.40 (dt, J=8.5, 2.7 Hz, 2H), 0.07 (s, 1H).

### Compounds 628 and 628A

### 3-(1-oxo-4-{[(1r,4r)-4-({[1-(trifluoromethyl)cyclopropyl]methyl}amino)cyclohexyl](5,5,5-trifluoropentyl)amino}-3H-isoindol-2-yl)piperidine-2,6-dione; trifluoroacetate and 3-(1-oxo-4-{[(1s,4s)-4-({[1-(trifluoromethyl)cyclopropyl]methyl}amino)cyclohexyl](5,5,5-trifluoropentyl)amino}-3H-isoindol-2-yl)piperidine-2,6-dione; trifluoroacetate

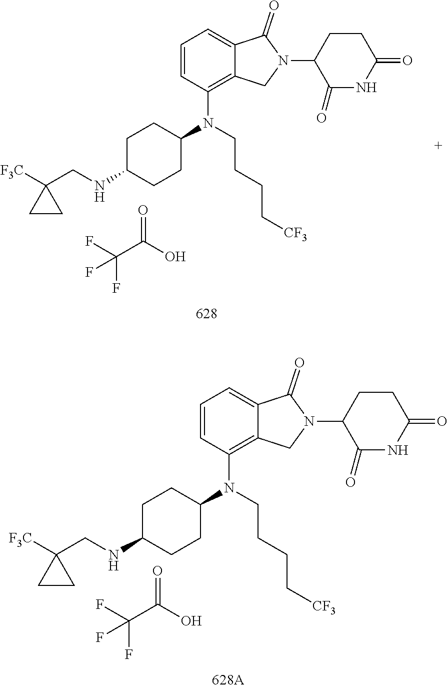

The compounds were prepared by the same procedure described as preparation of Compounds 554 and 554A above.

Compound 628: LC-MS: (ES, m/z): [M+1]=603.35; ¹H NMR (400 MHz, DMSO-d6) δ 10.97 (s, 1H), 8.62 (s, 2H), 7.44 (t, J=7.6 Hz, 1H), 7.33 (t, J=8.2 Hz, 2H), 5.13 (dd, J=13.2, 5.1 Hz, 1H), 4.32 (d, J=17.3 Hz, 1H), 4.22 (d, J=17.2 Hz, 1H), 3.25 (d, J=11.9 Hz, 2H), 3.13 (t, J=7.0 Hz, 2H), 3.06-3.01 (m, 1H), 3.01-2.86 (m, 2H), 2.62 (d, J=3.7 Hz, 1H), 2.60-2.52 (m, 1H), 2.49-2.41 (m, 1H), 2.18 (tdd, J=11.5, 9.7, 5.4 Hz, 2H), 2.08 (d, J=11.3 Hz, 2H), 1.98 (ddq, J=10.6, 5.2, 2.9, 2.0 Hz, 1H), 1.83 (d, J=12.0 Hz, 2H), 1.61-1.47 (m, 2H), 1.43 (td, J=11.7, 10.1, 5.5 Hz, 3H), 1.36-1.22 (m, 2H), 1.13 (q, J=6.2, 4.7 Hz, 2H), 1.07 (d, J=5.0 Hz, 2H). ¹⁹F NMR (377 MHz, DMSO-d6) δ −64.83, −67.77, −73.99.

Compound 628A: LC-MS: (ES, m/z): [M+1]=603.35; ¹H NMR (400 MHz, DMSO-d6) δ 11.00 (s, 1H), 8.60 (s, 2H), 7.51-7.43 (m, 1H), 7.40 (ddd, J=9.2, 7.5, 1.3 Hz, 2H), 5.12 (dd, J=13.2, 5.2 Hz, 1H), 4.42 (d, J=17.3 Hz, 1H), 4.30 (d, J=17.2 Hz, 1H), 3.29 (t, J=5.5 Hz, 2H), 3.10 (td, J=15.1, 7.8 Hz, 3H), 2.94 (ddd, J=17.2, 13.6, 5.4 Hz, 1H), 2.70-2.54 (m, 1H), 2.43 (td, J=13.3, 4.6 Hz, 3H), 2.26-2.11 (m, 2H), 2.03 (ddd, J=13.9, 6.1, 3.8 Hz, 1H), 1.82 (s, 2H), 1.73 (dd, J=26.2, 9.8 Hz, 3H), 1.48 (d, J=9.9 Hz, 2H), 1.37 (dh, J=12.7, 5.8 Hz, 4H), 1.20-1.11 (m, 2H), 1.10 (d, J=7.6 Hz, 2H). ¹⁹F NMR (377 MHz, DMSO-d₆) δ −64.83, −67.71,-74.44.

### Compounds 634 and 635

### 3-{4-[(2-cyclopropylethyl)[(1r,4r)-4-amino-4-methylcyclohexyl]amino]-1-oxo-3H-isoindol-2-yl}piperidine-2,6-dione and 3-{4-[(2-cyclopropylethyl)[(1s,4s)-4-amino-4-methylcyclohexyl]amino]-1-oxo-3H-isoindol-2-yl}piperidine-2,6-dione

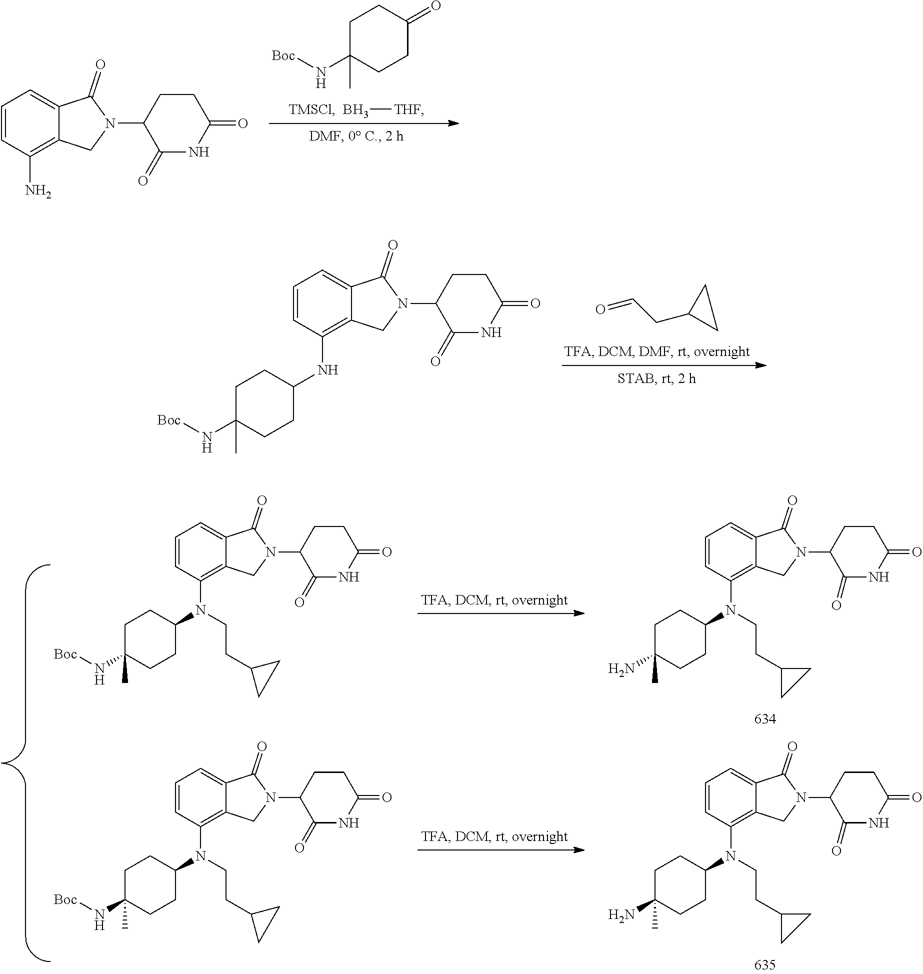

### Step-1: tert-butyl N-(4-{[2-(2,6-dioxopiperidin-3-yl)-1-oxo-3H-isoindol-4-yl]amino}-1-methylcyclohexyl)carbaminate

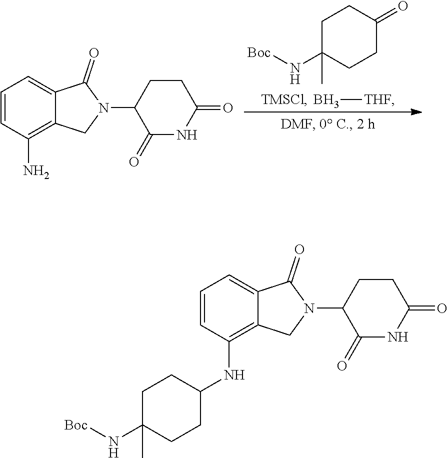

A solution of lenalidomide (500 mg, 1.92 mmol, 1 equiv) and tert-butyl N-(1-methyl-4-oxocyclohexyl)carbamate (657 mg, 2.89 mmol, 1.5 equiv) in DMF (10 mL) was treated with TMSCl (2.1 g, 19.3 mmol, 10 equiv) for 1 h at 0° C. under nitrogen atmosphere followed by the addition of BH₃-THF (9.6 mL, 9.6 mmol, 5 equiv) dropwise at 0° C. The resulting mixture was stirred for additional 1 h at room temperature. The reaction was quenched by the addition of water (3 mL) at room temperature. The resulting mixture was diluted with water at room temperature and extracted with EtOAc (2×50 mL). The combined organic layers were washed with brine (2×100 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in tert-butyl N-(4-{[2-(2,6-dioxopiperidin-3-yl)-1-oxo-3H-isoindol-4-yl]amino}-1-methylcyclohexyl)carbamate (900 mg, 99%) as a white solid.

### Step-2: tert-butyl N-[(1r,4r)-4-[(2-cyclopropylethyl)[2-(2,6-dioxopiperidin-3-yl)-1-oxo-3H-isoindol-4-yl]amino]-1-methylcyclohexyl]carbamate and tert-butyl N-[(1s,4s)-4-[(2-cyclopropylethyl)[2-(2,6-dioxopiperidin-3-yl)-1-oxo-3H-isoindol-4-yl]amino]-1-methylcyclohexyl]carbamate

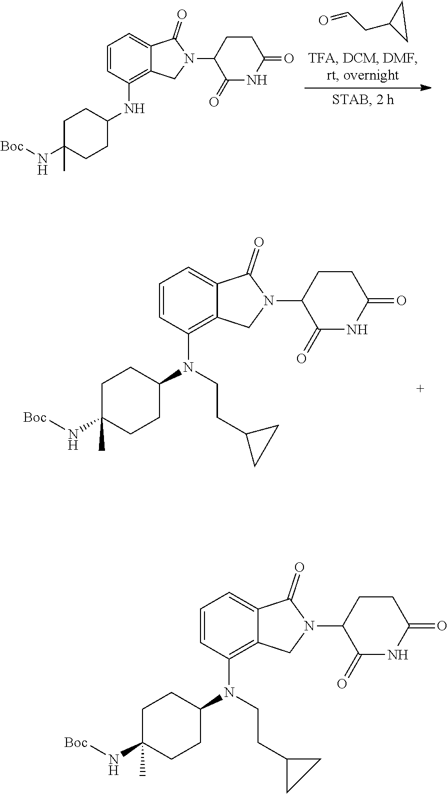 [SMILES: CC1(NC(=O)OC(C)(C)C)CCC(Nc2cccc3c2CN(C2CCC(=O)NC2=O)C3=O)CC1 ; O=CCC1CC1 ; CC(C)(C)OC(=O)N[C@]1(C)CC[C@@H](N(CCC2CC2)c2cccc3c2CN(C2CCC(=O)NC2=O)C3=O)CC1 ; CC(C)(C)OC(=O)N[C@]1(C)CC[C@H](N(CCC2CC2)c2cccc3c2CN(C2CCC(=O)NC2=O)C3=O)CC1]

A solution of tert-butyl N-(4-{[2-(2,6-dioxopiperidin-3-yl)-1-oxo-3H-isoindol-4-yl]amino}-1-methylcyclohexyl)carbamate (500 mg, 1.06 mmol, 1 equiv) and 2-cyclopropylacetaldehyde (1787 mg, 21.2 mmol, 20 equiv) in DCM (12.5 mL) and DMF (3 mL) was treated with TFA (363 mg, 3.18 mmol, 3 equiv) for overnight at room temperature under nitrogen atmosphere followed by the addition of STAB (1125 mg, 5.3 mmol, 5 equiv) in portions at 0° C. The resulting mixture was stirred for an additional 2 h at room temperature. The resulting mixture was diluted with water at room temperature and extracted with EtOAc (2×20 mL). The combined organic layers were washed with brine (2×50 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in tert-butyl N-[(1r,4r)-4-[(2-cyclopropylethyl)[2-(2,6-dioxopiperidin-3-yl)-1-oxo-3H-isoindol-4-yl]amino]-1-methylcyclohexyl]carbamate (250 mg, 44%) as an off-white solid and tert-butyl N-[(1s,4s)-4-[(2-cyclopropylethyl)[2-(2,6-dioxopiperidin-3-yl)-1-oxo-3H-isoindol-4-yl]amino]-1-methylcyclohexyl]carbamate (200.0 mg, 35%) as an off-white solid.

### Step-3: 3-{4-[(2-cyclopropylethyl)[(1r,4r)-4-amino-4-methylcyclohexyl]amino]-1-oxo-3H-isoindol-2-yl}piperidine-2,6-dione

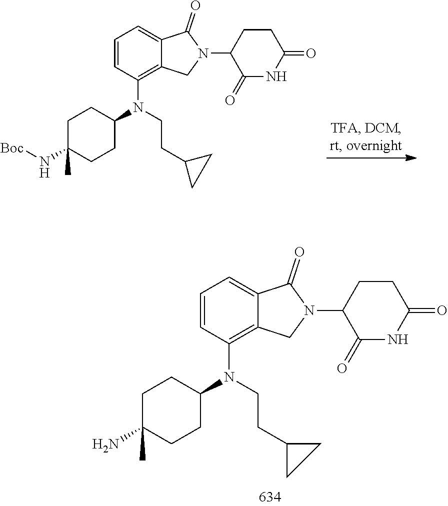 [SMILES: CC(C)(C)OC(=O)N[C@]1(C)CC[C@@H](N(CCC2CC2)c2cccc3c2CN(C2CCC(=O)NC2=O)C3=O)CC1 ; C[C@]1(N)CC[C@H](N(CCC2CC2)c2cccc3c2CN(C2CCC(=O)NC2=O)C3=O)CC1]

A solution of tert-butyl N-[(1r,4r)-4-[(2-cyclopropylethyl)[2-(2,6-dioxopiperidin-3-yl)-1-oxo-3H-isoindol-4-yl]amino]-1-methylcyclohexyl]carbamate (250 mg, 0.74 mmol, 1 equiv) in DCM (5 mL) was added TFA (1 mL) dropwise at 0° C. The resulting mixture was stirred for overnight at room temperature under. The resulting mixture was concentrated under reduced pressure. The residue was purified by reversed-phase flash chromatography with the following conditions: column, C18 silica gel; mobile phase, MeCN in Water (0.1% FA), 0% to 100% gradient in 10 min; detector, UV 254 nm. This resulted in 3-{4-[(2-cyclopropylethyl)[(1r,4r)-4-amino-4-methylcyclohexyl]amino]-1-oxo-3H-isoindol-2-yl}piperidine-2,6-dione (84.6 mg, 25%) as a yellow green solid.

Compound 634: LCMS: 439.20 (M+H). ¹H NMR (400 MHz, DMSO-d₆): δ 8.51 (s, 1H), 7.45 (t, J=7.7 Hz, 1H), 7.29 (dd, J=12.7, 7.7 Hz, 2H), 5.13 (dd, J=13.2, 5.1 Hz, 1H), 4.40 (d, J=17.2 Hz, 1H), 4.30 (d, J=17.1 Hz, 1H), 3.36 (dd, J=8.4, 6.1 Hz, 2H), 3.20-3.10 (m, 1H), 2.95 (ddd, J=17.1, 13.6, 5.4 Hz, 1H), 2.67-2.62 (m, 1H), 2.52-2.43 (m, 1H), 2.06 (dtd, J=12.8, 5.4, 2.3 Hz, 1H), 1.92 (t, J=12.1 Hz, 2H), 1.85-1.76 (m, 2H), 1.65-1.45 (m, 4H), 1.20 (d, J=13.9 Hz, 5H), 0.68 (tq, J=9.9, 3.5, 2.2 Hz, 1H), 0.43-0.30 (m, 2H).

### Step-4: 3-{4-[(2-cyclopropylethyl)[(1s,4s)-4-amino-4-methylcyclohexyl]amino]-1-oxo-3H-isoindol-2-yl}piperidine-2,6-dione

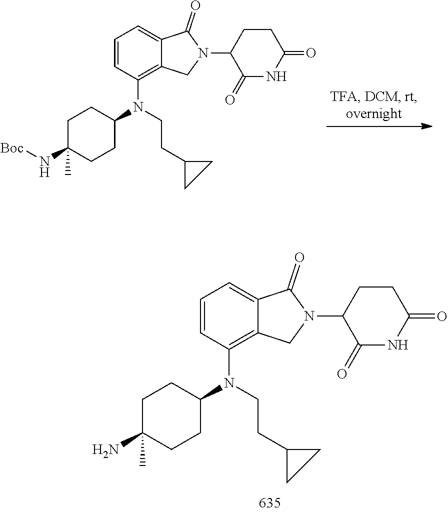 [SMILES: CC(C)(C)OC(=O)N[C@]1(C)CC[C@H](N(CCC2CC2)c2cccc3c2CN(C2CCC(=O)NC2=O)C3=O)CC1 ; C[C@]1(N)CC[C@@H](N(CCC2CC2)c2cccc3c2CN(C2CCC(=O)NC2=O)C3=O)CC1]

A solution of tert-butyl N-[(1s,4s)-4-[(2-cyclopropylethyl)[2-(2,6-dioxopiperidin-3-yl)-1-oxo-3H-isoindol-4-yl]amino]-1-methylcyclohexyl]carbamate (200 mg, 0.37 mmol, 1 equiv) (1 mL) in DCM (5 mL) was added TFA (1 mL) dropwise at 0° C. The resulting mixture was stirred for overnight at room temperature. The resulting mixture was concentrated under reduced pressure. The residue was purified by reversed-phase flash chromatography with the following conditions: column, C18 silica gel; mobile phase, MeCN in Water (0.1% FA), 0% to 100% gradient in 10 min; detector, UV 254 nm. This resulted in 3-{4-[(2-cyclopropylethyl)[(1s,4s)-4-amino-4-methylcyclohexyl]amino]-1-oxo-3H-isoindol-2-yl}piperidine-2,6-dione (55.7 mg, 34%) as a white solid.

Compound 635: LCMS: 439.20 (M+H). ¹H NMR (400 MHz, DMSO-d₆): δ 8.51 (s, 1H), 7.49 (t, J=7.6 Hz, 1H), 7.35 (dd, J=18.0, 7.6 Hz, 2H), 5.18 (dd, J=13.1, 5.1 Hz, 1H), 4.41 (d, J=17.2 Hz, 1H), 4.31 (d, J=17.2 Hz, 1H), 3.29 (t, J=7.2 Hz, 2H), 3.05-2.91 (m, 1H), 2.75-2.58 (m, 2H), 2.07 (ddd, J=13.2, 5.9, 3.6 Hz, 1H), 1.86-1.56 (m, 8H), 1.31 (s, 3H), 1.27-1.17 (m, 2H), 0.71 (ddt, J=10.1, 7.1, 3.9 Hz, 1H), 0.45-0.32 (m, 2H), 0.07 (s, 2H).

### Compounds 636 and 636A

### 3-(1-oxo-4-{[(1r,4r)-4-[(3,3,3-trifluoropropyl)amino]cyclohexyl](5,5,5-trifluoropentyl)amino}-3H-isoindol-2-yl)piperidine-2,6-dione; trifluoroacetate and 3-(1-oxo-4-{[(1s,4s)-4-[(3,3,3-trifluoropropyl)amino]cyclohexyl](5,5,5-trifluoropentyl)amino}-3H-isoindol-2-yl)piperidine-2,6-dione; trifluoroacetate

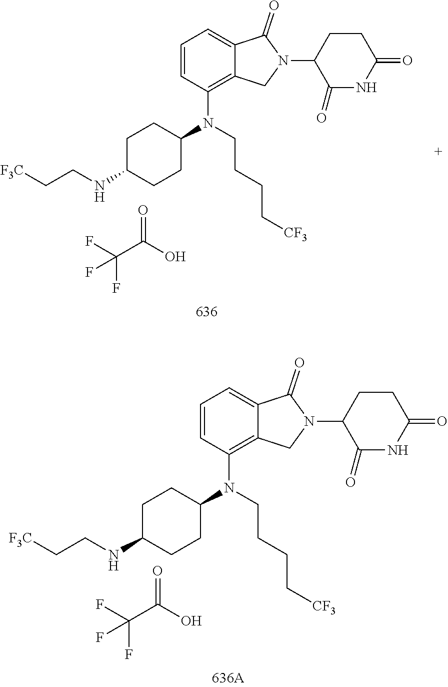

The compounds were prepared by the same procedure described as preparation of Compounds 554 and 554A above.

Compound 636: LC-MS: (ES, m/z): [M+1]=577.30; ¹H NMR (400 MHz, DMSO-d6) δ 10.97 (s, 1H), 8.71 (d, J=7.9 Hz, 2H), 7.44 (t, J=7.6 Hz, 1H), 7.34 (t, J=7.3 Hz, 2H), 5.12 (dd, J=13.2, 5.1 Hz, 1H), 4.33 (d, J=17.3 Hz, 1H), 4.23 (d, J=17.2 Hz, 1H), 3.23-2.99 (m, 6H), 2.92 (ddd, J=17.3, 13.6, 5.4 Hz, 1H), 2.76-2.52 (m, 4H), 2.48-2.40 (m, 1H), 2.26-2.12 (m, 2H), 2.11-1.94 (m, 3H), 1.88-1.78 (m, 2H), 1.55 (s, 1H), 1.50-1.37 (m, 3H), 1.37-1.35 (m, 1H), 1.31 (t, J=7.4 Hz, 2H). ¹⁹F NMR (377 MHz, DMSO-d6) δ −64.04, −64.83, −74.19.

Compound 636A: LC-MS: (ES, m/z): [M+1]=577.30; ¹H NMR (400 MHz, DMSO-d6) δ 10.98 (s, 1H), 8.70 (s, 2H), 7.45 (t, J=7.6 Hz, 1H), 7.36 (t, J=7.1 Hz, 2H), 5.11 (dd, J=13.2, 5.1 Hz, 1H), 4.39 (d, J=17.2 Hz, 1H), 4.28 (d, J=17.2 Hz, 1H), 3.38-3.05 (m, 6H), 2.92 (ddd, J=17.1, 13.6, 5.4 Hz, 1H), 2.75 (dq, J=17.0, 10.5 Hz, 2H), 2.60 (dd, J=17.3, 3.6 Hz, 1H), 2.44 (dt, J=13.3, 6.5 Hz, 3H), 2.20 (dd, J=7.9, 3.8 Hz, 1H), 2.17-2.11 (m, 1H), 2.02 (dtd, J=11.9, 6.8, 6.0, 3.3 Hz, 1H), 1.85 (s, 1H), 1.76 (dd, J=18.9, 6.5 Hz, 2H), 1.69 (dd, J=10.6, 3.5 Hz, 2H), 1.55 (s, 1H), 1.38 (dddd, J=23.4, 14.7, 8.9, 5.5 Hz, 4H). ¹⁹F NMR (377 MHz, DMSO-d6) δ −64.07, −64.86, −74.16.

### Compounds 639 and 639A

### 3-{4-[(2-cyclopropylethyl)[(1r,4r)-4-[(3,3,3-trifluoro-2,2-dimethylpropyl)amino]cyclohexyl]amino]-1-oxo-3H-isoindol-2-yl}piperidine-2,6-dione; bis(trifluoroacetic acid) and 3-{4-[(2-cyclopropylethyl)[(1s,4s)-4-[(3,3,3-trifluoro-2,2-dimethylpropyl)amino]cyclohexyl]amino]-1-oxo-3H-isoindol-2-yl}piperidine-2,6-dione; tris(trifluoroacetic acid)

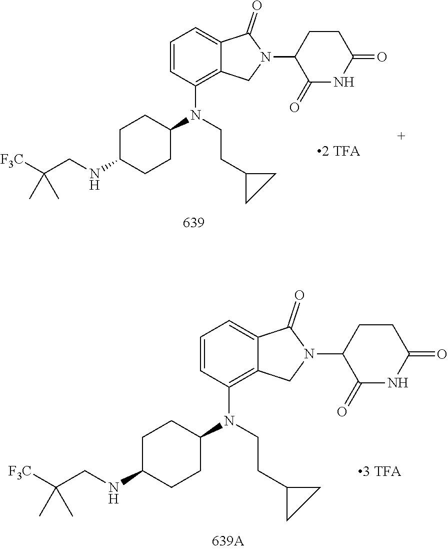 [SMILES: CC(C)(CN[C@H]1CC[C@H](N(CCC2CC2)c2cccc3c2CN(C2CCC(=O)NC2=O)C3=O)CC1)C(F)(F)F ; CC(C)(CN[C@H]1CC[C@@H](N(CCC2CC2)c2cccc3c2CN(C2CCC(=O)NC2=O)C3=O)CC1)C(F)(F)F]

The compounds were prepared by the general procedure described in the preparation of Compounds 515 and 515A using the appropriate amine.

Compound 639: LCMS: 549.2 (M+H). Method: Mobile Phase A: Water/5 mM NH4HCO3. Mobile Phase B: Acetonitrile. Flow rate: 1.5 mL/min. COLUMN: HPH-C18 (50×3.0) mm, 4μ. Rt (min): 1.269; Area %—99.75. ¹H NMR (400 MHz, DMSO-d6): δ 10.97 (s, 1H), 8.47 (s, 2H), 7.44 (t, J=7.6 Hz, 1H), 7.31 (dd, J=14.7, 7.6 Hz, 2H), 5.19-5.06 (m, 1H), 4.29 (d, J=24.5 Hz, 2H), 3.27-3.03 (m, 6H), 3.00-2.86 (m, 1H), 2.62 (d, J=3.4 Hz, 1H), 2.60-2.54 (m, 1H), 2.48 (s, 1H), 2.18-2.07 (m, 2H), 2.00 (ddd, J=7.2, 5.7, 3.0 Hz, 1H), 1.86 (s, 2H), 1.55 (q, J=15.4 Hz, 4H), 1.23 (s, 6H), 1.15 (qd, J=6.9, 2.3 Hz, 2H), 0.71-0.59 (m, 1H), 0.38-0.29 (m, 2H), −0.02-−0.11 (m, 2H). ¹⁹F NMR (377 MHz, DMSO-d6): δ −74.60 (d, J=8.1 Hz,2), −76.67 (s,1).

Compound 639A: LCMS: 549.2 (M+H). Method: Mobile Phase A: Water/5 mM NH4HCO3. Mobile Phase B: Acetonitrile. Flow rate: 1.5 mL/min. COLUMN: HPH-C18 (50×3.0) mm, 4μ. Rt (min): 1.539; Area %—98.70. ¹H NMR (400 MHz, DMSO-d6): δ 10.99 (s, 1H), 8.36 (d, J=75.7 Hz, 2H), 7.49-7.36 (m, 3H), 5.13 (dd, J=13.2, 5.1 Hz, 1H), 4.48-4.39 (m, 1H), 4.30 (d, J=17.2 Hz, 1H), 3.48 (s, 1H), 3.13 (d, J=7.0 Hz, 5H), 3.02-2.85 (m, 1H), 2.62 (t, J=18.3 Hz, 1H), 2.38 (d, J=39.1 Hz, 1H), 2.10-1.99 (m, 1H), 1.81 (d, J=38.6 Hz, 6H), 1.50 (d, J=12.7 Hz, 2H), 1.25 (s, 6H), 1.19 (s, 2H), 0.56 (q, J=6.9 Hz, 1H), 0.33 (dq, J=5.0, 3.4 Hz, 2H), −0.09 (t, J=4.5 Hz, 2H). ¹⁹F NMR (377 MHz, DMSO-d6): δ −74.50 (d, J=113.7 Hz,3), −76.60 (d, J=64.7 Hz,1).

### Compounds 642 and 642A

### 3-(4-[(2-cyclopropylethyl)[(1r,4r)-4-[(4,4,4-trifluorobutyl)amino]cyclohexyl]amino]-1-oxo-3H-isoindol-2-ylpiperidine-2,6-dione; bis(trifluoroacetic acid) and 3-(4-[(2-cyclopropylethyl)[(1s,4s)-4-[(4,4,4-trifluorobutyl)amino]cyclohexyl]amino]-1-oxo-3H-isoindol-2-ylpiperidine-2,6-dione; bis(trifluoroacetic acid)

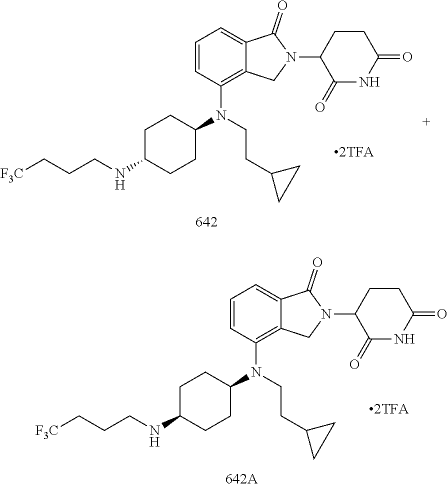 [SMILES: O=C1CCC(N2Cc3c(cccc3N(CCC3CC3)[C@H]3CC[C@H](NCCCC(F)(F)F)CC3)C2=O)C(=O)N1 ; O=C1CCC(N2Cc3c(cccc3N(CCC3CC3)[C@H]3CC[C@@H](NCCCC(F)(F)F)CC3)C2=O)C(=O)N1]

The compounds were prepared by the general procedure described in the preparation of Compounds 515 and 515A using the appropriate amine.

Compound 642: LCMS: 535.50 (M+H). Method: Mobile Phase A: Water/5 mM NH4HCO3. Mobile Phase B: Acetonitrile. Flow rate: 1.5000 mL/min COLUMN: Poroshell HPH-C18 (50×3.0) mm 2.7 μm. Rt (min): 1.405; Area %—99.211. ¹H NMR (400 MHz, DMSO-d6): PH-PNTX-C1-0642-0: δ 10.96 (s, 1H), 8.38 (s, 1H), 7.43 (t, J=7.7 Hz, 1H), 7.30 (dd, J=14.8, 7.6 Hz, 2H), 5.12 (dd, J=13.2, 5.1 Hz, 1H), 4.34 (d, J=17.2 Hz, 2H), 4.23 (d, J=17.2 Hz, 2H), 3.20 (t, J=7.3 Hz, 2H), 3.08 (d, J=12.0 Hz, 2H), 2.98 (s, 2H), 2.90 (dd, J=13.0, 4.7 Hz, 1H), 2.60 (d, J=16.9 Hz, 1H), 2.39 (dt, J=11.4, 7.9 Hz, 3H), 2.02 (dd, J=22.9, 9.5 Hz, 3H), 1.86-1.74 (m, 4H), 1.59 (d, J=12.9 Hz, 2H), 1.35 (d, J=12.7 Hz, 2H), 1.15 (q, J=7.5 Hz, 2H), 0.65 (d, J=7.1 Hz, 2H), 0.38-0.29 (m, 2H), −0.06 (d, J=4.8 Hz, 2H).

Compound 642A: LCMS: 535.55 (M+H). Method: Mobile Phase A: Water/5 mM NH4HCO3. Mobile Phase B: Acetonitrile: Flow rate: 1.5000 mL/min COLUMN: Poroshell HPH-C18(50×3.0) mm 2.7 um. Rt (min): 1.462; Area %—99.716. ¹H NMR (400 MHz, DMSO-d₆): PH-PNTX-C1-0642-0A: ¹H NMR (400 MHz, DMSO-d6) δ 10.98 (s, 1H), 8.33 (s, 2H), 7.43 (d, J=7.6 Hz, 1H), 7.32 (d, J=7.9 Hz, 2H), 5.11 (d, J=8.1 Hz, 1H), 4.28 (s, 1H), 3.30 (s, 1H), 3.21 (s, 1H), 3.02 (s, 2H), 2.93-2.89 (m, 1H), 2.58 (s, 1H), 2.54 (s, 2H), 2.42-2.37 (m, 4H), 2.02 (d, J=5.6 Hz, 1H), 1.83 (d, J=10.3 Hz, 6H), 1.70 (s, 2H), 1.56 (s, 2H), 1.16 (s, 2H), 0.60 (d, J=6.6 Hz, 1H), 0.34 (d, J=3.2 Hz, 2H), −0.06-−0.07 (m, 2H). 19F NMR (377 MHz, DMSO-d6): PH-PNTX-C1-0642-0A: 6-64.99 (d, J=4.4 Hz, 1), −74.73 (s,2).

### Compounds 643 and 643A

### 3-{4-[(2-cyclopropylethyl)[(1r,4r)-4-[(3,3-difluoropropyl)amino]cyclohexyl]amino]-1-oxo-3H-isoindol-2-yl}piperidine-2,6-dione; trifluoroacetic acid and 3-{4-[(2-cyclopropylethyl)[(1s,4s)-4-[(3,3-difluoropropyl)amino]cyclohexyl]amino]-1-oxo-3H-isoindol-2-yl}piperidine-2,6-dione; bis(trifluoroacetic acid)

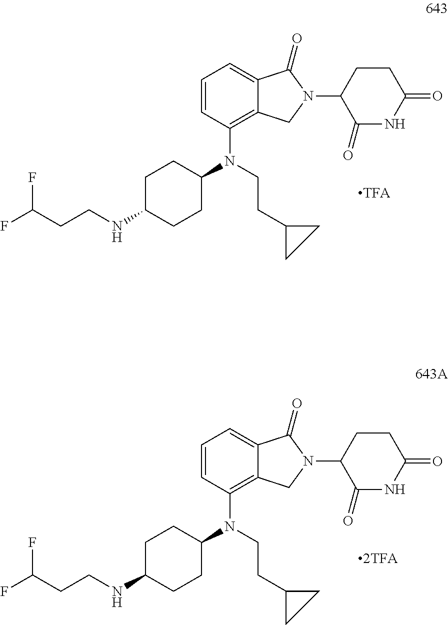 [SMILES: O=C1CCC(N2Cc3c(cccc3N(CCC3CC3)[C@H]3CC[C@H](NCCC(F)F)CC3)C2=O)C(=O)N1 ; O=C1CCC(N2Cc3c(cccc3N(CCC3CC3)[C@H]3CC[C@@H](NCCC(F)F)CC3)C2=O)C(=O)N1]

The compounds were prepared by the general procedure described in the preparation of Compounds 515 and 515A using the appropriate amine.

Compound 643: LCMS: 503.15 (M+H). Method: Mobile Phase A: Water/5 mM NH₄HCO₃. Mobile Phase B: Acetonitrile. Flow rate: 1.5 mL/min. COLUMN: EVO C18 (50×3.0) mm, 2.6μ. Rt (min): 1.475; Area %—96.67. ¹H NMR (400 MHz, DMSO-d6): δ 11.02 (s, 1H), 8.58 (s, 2H), 7.48 (t, J=7.7 Hz, 1H), 7.35 (dd, J=14.7, 7.6 Hz, 2H), 6.34 (dt, J=55.9, 4.2 Hz, 1H), 5.18 (dd, J=13.2, 5.1 Hz, 1H), 4.40 (d, J=17.2 Hz, 1H), 4.29 (d, J=17.1 Hz, 1H), 3.25 (t, J=7.4 Hz, 2H), 3.14 (d, J=12.8 Hz, 4H), 2.98 (ddd, J=17.8, 13.6, 5.4 Hz, 1H), 2.70-2.61 (m, 1H), 2.53-2.48 (m, 1H), 2.40-2.00 (m, 6H), 1.90 (s, 1H), 1.62 (t, J=13.6 Hz, 2H), 1.42 (d, J=14.1 Hz, 2H), 1.29 (s, 1H), 1.20 (q, J=7.3 Hz, 2H), 0.70 (dq, J=13.1, 6.3, 5.6 Hz, 1H), 0.44-0.35 (m, 2H), 0.06 (s, 2H). ¹⁹F NMR (377 MHz, DMSO-d6): δ −74.02 (s,1), −116.45 (s,1)

Compound 643A: LCMS: 503.20 (M+H). Method: Mobile Phase A: Water/5 mM NH₄HCO₃. Mobile Phase B: Acetonitrile. Flow rate: 1.5 mL/min. COLUMN: EVO C18 (50×3.0) mm, 2.6μ. Rt (min): 1.542; Area % —98.79. ¹H NMR (400 MHz, DMSO-d6): δ 10.98 (s, 1H), 8.49 (s, 2H), 7.44 (t, J=7.6 Hz, 1H), 7.34 (dd, J=14.3, 7.6 Hz, 2H), 6.35-6.08 (m, 1H), 5.11 (dd, J=13.3, 5.1 Hz, 1H), 4.39 (d, J=17.2 Hz, 2H), 4.28 (d, J=17.2 Hz, 2H), 3.35-3.17 (m, 4H), 2.92 (ddd, J=17.9, 13.4, 5.3 Hz, 1H), 2.65-2.56 (m, 1H), 2.43 (dd, J=13.1, 4.5 Hz, 1H), 2.32-2.22 (m, 2H), 2.07-1.99 (m, 1H), 1.76 (d, J=52.6 Hz, 6H), 1.56 (s, 2H), 1.24 (s, 1H), 1.17 (q, J=7.2 Hz, 2H), 0.64-0.56 (m, 1H), 0.38-0.29 (m, 2H), −0.07 (q, J=4.7 Hz, 2H). ¹⁹F NMR (377 MHz, DMSO-d6): δ −74.09 (s,2), −116.46 (s,1).

### Compounds 644 and 644A

### 3-(4-[(2-cyclopropylethyl)[(1r,4r)-4-[(2,2,3,3,3-pentafluoropropyl)amino]cyclohexyl]amino]-1-oxo-3H-isoindol-2-ylpiperidine-2,6-dione and 3-(4-[(2-cyclopropylethyl)[(1s,4s)-4-[(2,2,3,3,3-pentafluoropropyl)amino]cyclohexyl]amino]-1-oxo-3H-isoindol-2-ylpiperidine-2,6-dione

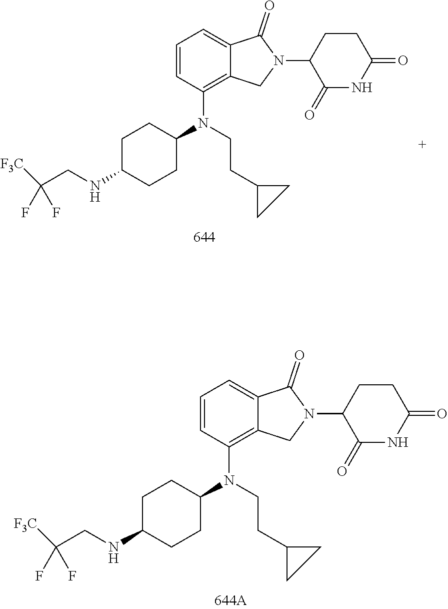 [SMILES: O=C1CCC(N2Cc3c(cccc3N(CCC3CC3)[C@H]3CC[C@H](NCC(F)(F)C(F)(F)F)CC3)C2=O)C(=O)N1 ; O=C1CCC(N2Cc3c(cccc3N(CCC3CC3)[C@H]3CC[C@@H](NCC(F)(F)C(F)(F)F)CC3)C2=O)C(=O)N1]

The compounds were prepared by the general procedure described in the preparation of Compounds 515 and 515A using the appropriate amine.

Compound 644: LCMS: 557.35 (M+H). Method: Mobile Phase A: Water/5 mM NH4HCO3, Mobile Phase B: Acetonitrile. Flow rate: 1.5000 mL/min. COLUMN: Poroshell HPH-C18 (50 x3.0) mm 2.7μ. Rt (min): 1.460; Area % —98.741. ¹H NMR (400 MHz, DMSO-d₆): PH-PNTX-C1-0644-0: ¹H NMR (400 MHz, DMSO-d6) δ 10.96 (s, 1H), 7.43 (t, J=7.6 Hz, 1H), 7.30 (dd, J=15.3, 7.6 Hz, 2H), 5.12 (dd, J=13.2, 5.1 Hz, 1H), 4.36-4.21 (m, 4H), 3.20 (t, J=7.2 Hz, 2H), 3.08 (s, 2H), 2.95-2.87 (m, 1H), 2.62 (s, 1H), 2.12 (s, 2H), 2.04-1.96 (m, 1H), 1.85 (s, 2H), 1.55 (d, J=12.9 Hz, 2H), 1.39 (s, 2H), 1.14 (dt, J=9.0, 4.5 Hz, 2H), 0.63 (td, J=7.3, 3.8 Hz, 1H), 0.38-0.29 (m, 2H), −0.07 (dt, J=5.6, 2.8 Hz, 2H).

Compound 644A: LCMS: 557.35 (M+H). Method: Mobile Phase A: Water/5 mM NH₄HCO₃, Mobile Phase B: Acetonitrile. Flow rate: 1.5000 mL/min. COLUMN: Poroshell HPH-C18 (50 x3.0) mm 2.7μ. Rt (min): 1.504; Area % —99.650. ¹H NMR (400 MHz, DMSO-d₆): PH-PNTX-C1-0644-0A: ¹H NMR (400 MHz, DMSO-d6) δ 10.98 (s, 1H), 7.43 (t, J=7.6 Hz, 1H), 7.33 (d, J=15.8 Hz, 2H), 5.10 (dd, J=13.2, 5.1 Hz, 1H), 4.44-4.24 (m, 3H), 3.34-3.16 (m, 4H), 2.96-2.87 (m, 1H), 2.60 (d, J=17.5 Hz, 2H), 2.45 (d, J=4.4 Hz, 1H), 2.06-1.99 (m, 1H), 1.92-1.75 (m, 4H), 1.63 (s, 2H), 1.47 (s, 2H), 1.17 (d, J=7.5 Hz, 2H), 0.60 (d, J=5.7 Hz, 1H), 0.38-0.29 (m, 2H), −0.07 (td, J=5.3, 3.8 Hz, 2H).

### Compounds 645 and 645A

### 3-{4-[(2-cyclopropylethyl)[(1r,4r)-4-({2-[1-(trifluoromethyl)cyclopropyl]ethyl}amino)cyclohexyl]amino]-1-oxo-3H-isoindol-2-yl}piperidine-2,6-dione trifluoroacetic acid and 3-{4-[(2-cyclopropylethyl)[(1s,4s)-4-({2-[1-(trifluoromethyl)cyclopropyl]ethyl}amino)cyclohexyl]amino]-1-oxo-3H-isoindol-2-yl}piperidine-2,6-dione trifluoroacetic acid

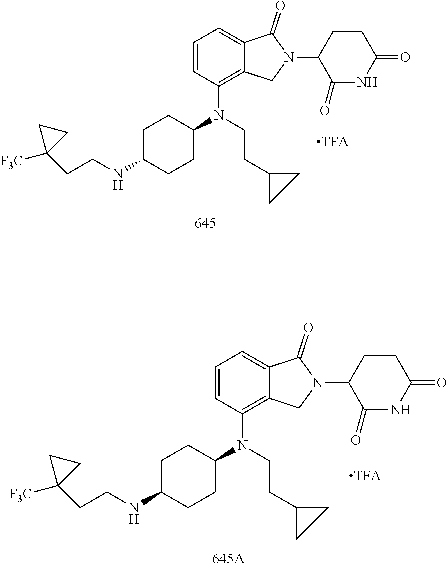 [SMILES: O=C1CCC(N2Cc3c(cccc3N(CCC3CC3)[C@H]3CC[C@H](NCCC4(C(F)(F)F)CC4)CC3)C2=O)C(=O)N1 ; O=C1CCC(N2Cc3c(cccc3N(CCC3CC3)[C@H]3CC[C@@H](NCCC4(C(F)(F)F)CC4)CC3)C2=O)C(=O)N1]

The compounds were prepared by the general procedure described in the preparation of Compounds 515 and 515A using the appropriate amine.

Compound 645: LC-MS: (ES, m/z): [M+1]=561.35 ¹H NMR (400 MHz, DMSO-d₆) δ 10.96 (s, 1H), 8.40 (s, 2H), 7.43 (t, J=7.7 Hz, 1H), 7.29 (dd, J=15.3, 7.6 Hz, 2H), 5.12 (dd, J=13.2, 5.1 Hz, 1H), 4.23 (d, J=17.1 Hz, 2H), 3.24-3.14 (m, 2H), 3.14-3.04 (m, 5H), 2.92 (ddd, J=17.3, 13.5, 5.3 Hz, 1H), 2.64-2.52 (m, 3H), 2.09-1.94 (m, 3H), 1.87 (dd, J=10.4, 6.7 Hz, 4H), 1.57 (d, J=13.6 Hz, 2H), 1.42-1.34 (m, 2H), 1.14 (q, J=7.2 Hz, 2H), 1.00-0.86 (m, 2H), 0.84 (d, J=5.9 Hz, 2H), 0.63 (pd, J=7.3, 3.9 Hz, 1H), 0.38-0.27 (m, 2H), −0.06 (dd, J=5.0, 1.5 Hz, 2H). ¹⁹F NMR (377 MHz, DMSO-d6) δ −68.38 (s,1), −74.29 (s,1).

Compound 645A: LC-MS: (ES, m/z): [M+1]=561.35 ¹H NMR (400 MHz, DMSO-d₆) δ 10.98 (s, 1H), 8.34 (s, 2H), 7.44 (t, J=7.6 Hz, 1H), 7.38-7.28 (m, 2H), 5.11 (dd, J=13.2, 5.1 Hz, 1H), 4.39 (d, J=17.3 Hz, 2H), 3.20 (dp, J=13.9, 7.4, 6.9 Hz, 4H), 3.06-2.98 (m, 2H), 2.92 (ddd, J=17.3, 13.6, 5.4 Hz, 1H), 2.65-2.56 (m, 1H), 2.49-2.38 (m, 1H), 2.07-1.91 (m, 1H), 1.83 (s, 2H), 1.83-1.75 (m, 4H), 1.68 (d, J=12.5 Hz, 2H), 1.55 (s, 2H), 1.01-0.90 (m, 2H), 0.86 (p, J=4.6, 3.9 Hz, 2H), 0.60 (tt, J=8.4, 3.9 Hz, 1H), 0.33 (dt, J=8.5, 2.7 Hz, 2H), −0.07 (h, J=3.5 Hz, 2H). ¹⁹F NMR (377 MHz, DMSO-d₆) δ −68.42 (s,1), −74.20 (s,1).

### Compounds 646 and 646A

### 3-{4-[(2-cyclopropylethyl)[(1r,4r)-4-{[3-(trifluoromethyl)bicyclo[1.1.1]pentan-1-yl]amino}cyclohexyl]amino]-1-oxo-3H-isoindol-2-yl}piperidine-2,6-dione; trifluoroacetic acid and 3-{4-[(2-cyclopropylethyl)[(1s,4s)-4-{[3-(trifluoromethyl)bicyclo[1.1.1]pentan-1-yl]amino}cyclohexyl]amino]-1-oxo-3H-isoindol-2-yl}piperidine-2,6-dione; trifluoroacetic acid

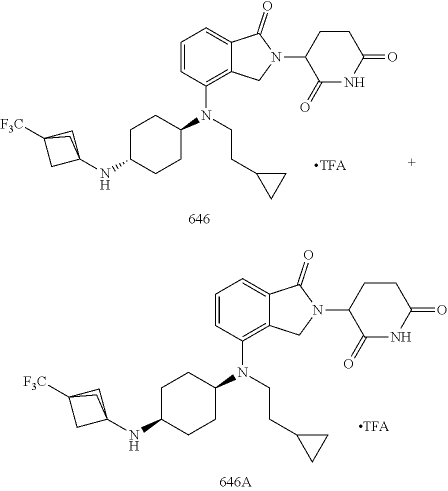 [SMILES: O=C1CCC(N2Cc3c(cccc3N(CCC3CC3)[C@H]3CC[C@H](NC45CC(C(F)(F)F)(C4)C5)CC3)C2=O)C(=O)N1 ; O=C1CCC(N2Cc3c(cccc3N(CCC3CC3)[C@H]3CC[C@@H](NC45CC(C(F)(F)F)(C4)C5)CC3)C2=O)C(=O)N1]

The compounds were prepared by the general procedure described in the preparation of Compounds 515 and 515A using the appropriate amine.

Compound 646: LCMS: 559.30 (M+H). Method: Mobile Phase A: Water/5 mM NH₄HCO₃. Mobile Phase B: Acetonitrile. Flow rate: 1.5 mL/min. COLUMN: EVO C18 (50×3.0) mm, 2.6μ. Rt (min): 1.579; Area %—98.97. ¹H NMR (400 MHz, DMSO-d6): δ 11.02 (s, 1H), 9.69 (s, 2H), 7.48 (t, J=7.6 Hz, 1H), 7.35 (dd, J=14.9, 7.6 Hz, 2H), 5.17 (dd, J=13.2, 5.1 Hz, 1H), 4.40 (d, J=17.2 Hz, 1H), 4.29 (d, J=17.1 Hz, 1H), 3.29-3.11 (m, 4H), 2.97 (ddd, J=17.0, 13.5, 5.4 Hz, 1H), 2.69-2.58 (m, 1H), 2.54-2.49 (m, 1H), 2.43 (s, 6H), 2.15-2.11 (m, 1H), 2.10 (s, 1H), 2.08-2.01 (m, 1H), 1.87 (s, 2H), 1.69 (d, J=11.0 Hz, 2H), 1.53-1.37 (m, 2H), 1.29 (s, 1H), 1.20 (q, J=7.3 Hz, 2H), 0.68 (tq, J=7.8, 3.2, 2.3 Hz, 1H), 0.44-0.33 (m, 2H), 0.05 (d, J=1.1 Hz, 2H). ¹⁹F NMR (377 MHz, DMSO-d6): δ −69.58 (s,1), −74.28 (s,1).

Compound 646A: LCMS: 559.30 (M+H). Method: Mobile Phase A: Water/5 mM NH₄HCO₃. Mobile Phase B: Acetonitrile. Flow rate: 1.5 mL/min. COLUMN: EVO C18 (50×3.0) mm, 2.6μ. Rt (min): 1.630; Area % —99.61. ¹H NMR (400 MHz, DMSO-d6): δ 10.98 (s, 1H), 9.58 (s, 2H), 7.44 (t, J=7.6 Hz, 1H), 7.33 (dd, J=13.7, 7.6 Hz, 2H), 5.11 (dd, J=13.2, 5.1 Hz, 1H), 4.39 (d, J=17.2 Hz, 1H), 4.27 (d, J=17.2 Hz, 1H), 3.32 (s, 2H), 3.20 (hept, J=7.1 Hz, 2H), 2.92 (ddd, J=16.9, 13.5, 5.4 Hz, 1H), 2.65-2.56 (m, 1H), 2.49-2.44 (m, 1H), 2.39 (s, 6H), 2.07-1.98 (m, 1H), 1.88-1.65 (m, 6H), 1.59 (s, 2H), 1.24 (s, 1H), 1.17 (q, J=7.3 Hz, 2H), 0.64-0.57 (m, 1H), 0.34 (dt, J=8.0, 2.9 Hz, 2H), −0.07 (q, J=4.7 Hz, 2H). ¹⁹F NMR (377 MHz, DMSO-d6): δ −69.50 (s,1), −74.23 (s,1).

### Compounds 647 and 647A

### 3-{4-[(2-cyclopropylethyl)[(1r,4r)-4-{[2-(trifluoromethoxy)ethyl]amino}cyclohexyl]amino]-1-oxo-3H-isoindol-2-yl}piperidine-2,6-dione bis(trifluoroacetic acid and 3-{4-[(2-cyclopropylethyl)[(1s,4s)-4-{[2-(trifluoromethoxy)ethyl]amino}cyclohexyl]amino]-1-oxo-3H-isoindol-2-yl}piperidine-2,6-dione bis(trifluoroacetic acid

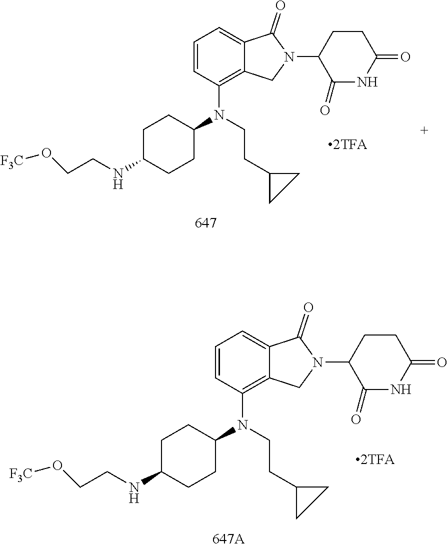 [SMILES: O=C1CCC(N2Cc3c(cccc3N(CCC3CC3)[C@H]3CC[C@H](NCCOC(F)(F)F)CC3)C2=O)C(=O)N1 ; O=C1CCC(N2Cc3c(cccc3N(CCC3CC3)[C@H]3CC[C@@H](NCCOC(F)(F)F)CC3)C2=O)C(=O)N1]

The compounds were prepared by the general procedure described in the preparation of Compounds 515 and 515A using the appropriate amine.

Compound 647: LC-MS: (ES, m/z): [M+1]=537.30. ¹H NMR (400 MHz, DMSO-d₆) δ 10.97 (s, 1H), 8.81 (q, J=6.4, 5.8 Hz, 2H), 7.43 (t, J=7.7 Hz, 1H), 7.30 (dd, J=14.6, 7.6 Hz, 2H), 5.12 (dd, J=13.2, 5.1 Hz, 1H), 4.34 (dd, J=10.7, 5.8 Hz, 4H), 3.32 (p, J=5.3 Hz, 2H), 3.20 (t, J=7.3 Hz, 2H), 3.15-3.04 (m, 2H), 2.92 (ddd, J=17.2, 13.6, 5.4 Hz, 1H), 2.65-2.51 (m, 1H), 2.46 (dd, J=13.2, 4.5 Hz, 1H), 2.12-2.05 (m, 2H), 2.00 (dtd, J=12.7, 5.2, 2.1 Hz, 1H), 1.90-1.81 (m, 2H), 1.65-1.50 (m, 2H), 1.41 (qt, J=13.1, 3.6 Hz, 2H), 1.14 (qd, J=7.2, 4.3 Hz, 2H), 0.63 (pd, J=7.4, 3.7 Hz, 1H), 0.38-0.27 (m, 2H), −0.06 (dd, J=4.9, 1.5 Hz, 2H). ¹⁹F NMR (377 MHz, DMSO-d₆) δ −59.32 (s,1), −74.44 (s,2)

Compound 647A: LC-MS: (ES, m/z): [M+1]=537.30 ¹H NMR (400 MHz, DMSO-d₆) δ 10.98 (s, 1H), 8.73 (s, 2H), 7.35 (dd, J=14.1, 7.5 Hz, 3H), 5.11 (dd, J=13.2, 5.1 Hz, 1H), 4.44-4.35 (m, 4H), 3.36 (q, J=10.0, 7.8 Hz, 3H), 3.19 (d, J=6.7 Hz, 3H), 2.92 (ddd, J=17.2, 13.6, 5.4 Hz, 1H), 2.60 (dt, J=17.2, 3.3 Hz, 1H), 2.51-2.39 (m, 1H), 2.08-1.97 (m, 1H), 1.89-1.77 (m, 4H), 1.75-1.66 (m, 2H), 1.54 (q, J=8.0 Hz, 2H), 1.26-1.10 (m, 2H), 0.59 (dtd, J=14.4, 7.8, 7.0, 3.6 Hz, 1H), 0.33 (dt, J=8.4, 2.8 Hz, 2H), −0.07 (h, J=3.6 Hz, 2H). ¹⁹F NMR (377 MHz, DMSO-d₆) δ −59.26 (s,1), −74.47 (s,2).

### Compounds 648 and 648A

### 3-(1-oxo-4-{propyl[(1r,4r)-4-[(3,3,3-trifluoropropyl)amino]cyclohexyl]amino}-3H-isoindol-2-yl)piperidine-2,6-dione trifluoroacetic acid and 3-(1-oxo-4-{propyl[(1s,4s)-4-[(3,3,3-trifluoropropyl)amino]cyclohexyl]amino}-3H-isoindol-2-yl)piperidine-2,6-dione trifluoroacetic acid

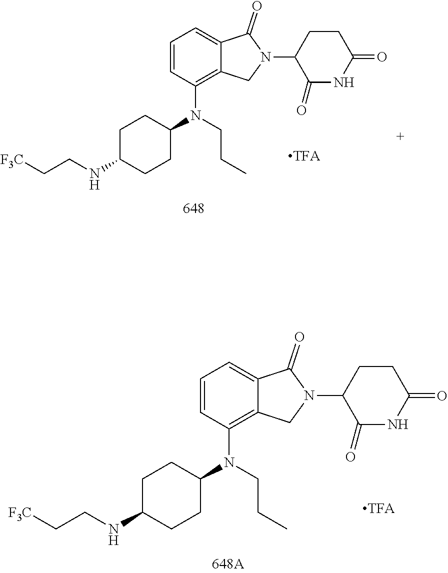 [SMILES: CCCN(c1cccc2c1CN(C1CCC(=O)NC1=O)C2=O)[C@H]1CC[C@H](NCCC(F)(F)F)CC1 ; CCCN(c1cccc2c1CN(C1CCC(=O)NC1=O)C2=O)[C@H]1CC[C@@H](NCCC(F)(F)F)CC1]

The compound was prepared by the same procedure described as preparation of Compounds 554 and 554A above.

Compound 648: LC-MS: (ES, m/z): [M+1]=495.30. ¹H NMR (400 MHz, DMSO-d₆) δ 10.97 (s, 1H), 8.73 (q, J=5.7 Hz, 2H), 7.43 (t, J=7.6 Hz, 1H), 7.31 (dd, J=13.0, 7.6 Hz, 2H), 5.12 (dd, J=13.2, 5.1 Hz, 1H), 4.34 (d, J=17.2 Hz, 1H), 4.24 (d, J=17.2 Hz, 1H), 3.18 (dq, J=11.9, 7.1 Hz, 2H), 3.07 (hept, J=6.7 Hz, 4H), 2.92 (ddd, J=17.8, 13.4, 5.4 Hz, 1H), 2.68 (td, J=11.3, 10.4, 4.5 Hz, 4H), 2.56 (dd, J=11.8, 4.7 Hz, 3H), 2.11-1.95 (m, 2H), 1.62-1.46 (m, 2H), 1.38 (dtd, J=16.5, 12.2, 6.4 Hz, 2H), 1.25 (h, J=7.0 Hz, 2H), 0.79 (t, J=7.3 Hz, 3H). ¹⁹F NMR (376 MHz, DMSO-d₆) δ −64.06 (s,1), −74.36 (s,1).

Compound 648A: LC-MS: (ES, m/z): [M+1]=495.30. ¹H NMR (400 MHz, DMSO-d₆) δ 10.99 (s, 1H), 8.63 (s, 2H), 7.44 (t, J=7.6 Hz, 1H), 7.35 (dd, J=10.9, 7.6 Hz, 2H), 5.11 (dd, J=13.2, 5.1 Hz, 1H), 4.40 (d, J=17.2 Hz, 1H), 4.28 (d, J=17.2 Hz, 1H), 3.36-3.23 (m, 4H), 3.20-3.07 (m, 2H), 3.04 (d, J=6.8 Hz, 1H), 2.82-2.67 (m, 2H), 2.60 (dt, J=17.0, 2.9 Hz, 1H), 2.43 (dd, J=13.2, 4.4 Hz, 1H), 2.10-1.97 (m, 1H), 1.73 (ddt, J=21.6, 12.4, 8.3 Hz, 6H), 1.55 (q, J=10.8, 9.1 Hz, 2H), 1.27 (dt, J=13.0, 6.5 Hz, 2H), 0.78 (t, J=7.3 Hz, 3H). ¹⁹F NMR (377 MHz, DMSO-d₆) δ −64.04 (s,1), −74.30 (s,1).

### Compounds 649 and 649A

### 3-{4-[(cyclobutylmethyl)[(1r,4r)-4-[(3,3,3-trifluoropropyl)amino]cyclohexyl]amino]-1-oxo-3H-isoindol-2-yl}piperidine-2,6-dione trifluoroacetic acid and 3-{4-[(cyclobutylmethyl)[(1s,4s)-4-[(3,3,3-trifluoropropyl)amino]cyclohexyl]amino]-1-oxo-3H-isoindol-2-yl}piperidine-2,6-dione trifluoroacetic acid

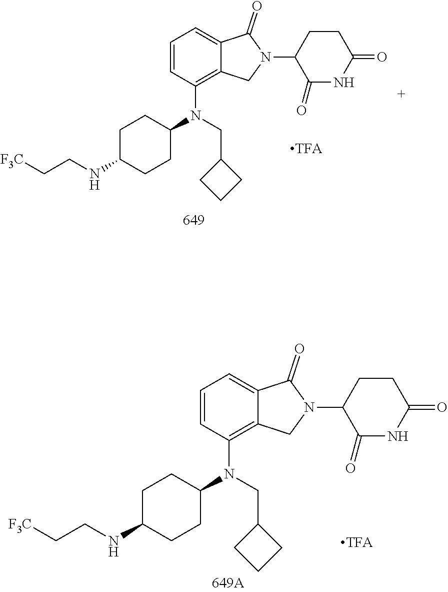 [SMILES: O=C1CCC(N2Cc3c(cccc3N(CC3CCC3)[C@H]3CC[C@H](NCCC(F)(F)F)CC3)C2=O)C(=O)N1 ; O=C1CCC(N2Cc3c(cccc3N(CC3CCC3)[C@H]3CC[C@@H](NCCC(F)(F)F)CC3)C2=O)C(=O)N1]

The compound was prepared by the same procedure described as preparation of Compounds 554 and 554A above.

Compound 649: LC-MS: (ES, m/z): [M+1]=521.35. ¹H NMR (400 MHz, DMSO-d₆) δ 10.97 (s, 1H), 8.69-8.63 (m, 2H), 7.43 (t, J=7.6 Hz, 1H), 7.39-7.30 (m, 2H), 5.12 (dd, J=13.2, 5.1 Hz, 1H), 4.29 (d, J=17.3 Hz, 1H), 4.19 (d, J=17.3 Hz, 1H), 3.13 (dtd, J=20.8, 14.9, 13.6, 7.5 Hz, 5H), 2.93 (dtd, J=17.2, 13.6, 12.5, 6.6 Hz, 2H), 2.76-2.55 (m, 3H), 2.20-1.95 (m, 4H), 1.85-1.76 (m, 6H), 1.53 (qt, J=18.7, 9.0 Hz, 4H), 1.35 (d, J=6.8 Hz, 2H). ¹⁹F NMR (377 MHz, DMSO-d₆) δ −64.03 (s,1), −74.23 (s,1).

Compound 649A: LC-MS: (ES, m/z): [M+1]=521.30. ¹H NMR (400 MHz, DMSO-d₆) δ 10.98 (s, 1H), 8.65 (s, 2H), 7.37 (ddd, J=10.4, 3H), 5.11 (dd, J=13.3, 5.1 Hz, 1H), 4.35 (d, J=17.3 Hz, 1H), 4.25 (d, J=17.2 Hz, 1H), 3.30-3.21 (m, 4H), 3.21-3.05 (m, 2H), 2.92 (ddd, J=18.1, 13.5, 5.4 Hz, 1H), 2.74 (q, J=10.4, 8.9 Hz, 2H), 2.65-2.56 (m, 1H), 2.49-2.39 (m, 1H), 2.20 (q, J=7.6 Hz, 1H), 2.07-1.98 (m, 1H), 1.87-1.64 (m, 10H), 1.60-1.47 (m, 4H). ¹⁹F NMR (377 MHz, DMSO-d₆) δ −64.02 (s,1), −74.19 (s,1).

### Compounds 650 and 650A

### 3-(4-{[(3,3-difluorocyclobutyl)methyl][(1r,4r)-4-[(3,3,3-trifluoropropyl)amino]cyclohexyl]amino}-1-oxo-3H-isoindol-2-yl)piperidine-2,6-dione and 3-(4-{[(3,3-difluorocyclobutyl)methyl][(1s,4s)-4-[(3,3,3-trifluoropropyl)amino]cyclohexyl]amino}-1-oxo-3H-isoindol-2-yl)piperidine-2,6-dione

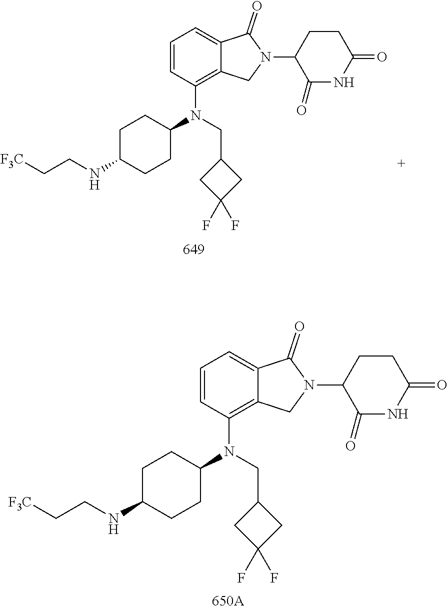 [SMILES: O=C1CCC(N2Cc3c(cccc3N(CC3CC(F)(F)C3)[C@H]3CC[C@H](NCCC(F)(F)F)CC3)C2=O)C(=O)N1 ; O=C1CCC(N2Cc3c(cccc3N(CC3CC(F)(F)C3)[C@H]3CC[C@@H](NCCC(F)(F)F)CC3)C2=O)C(=O)N1]

The compound was prepared by the same procedure described as preparation of Compounds 554 and 554A above.

Compound 650: LCMS: 557.15 (M+H). Method: Mobile Phase A: Water/5 mM NH₄HCO₃. Mobile Phase B: Acetonitrile. Flow rate: 1.5 mL/min. COLUMN: HPH-C18 (50×3.0) mm, 4.0μ. Rt (min): 1.784; Area %—99.46. ¹H NMR (400 MHz, DMSO-d6): δ 10.97 (s, 1H), 7.44 (dd, J=8.6, 6.5 Hz, 1H), 7.37 (d, J=7.6 Hz, 2H), 5.09 (dd, J=13.1, 5.1 Hz, 1H), 4.31 (d, J=17.3 Hz, 1H), 4.21 (d, J=17.3 Hz, 1H), 3.24-3.15 (m, 3H), 2.99-2.83 (m, 2H), 2.74 (t, J=7.5 Hz, 2H), 2.60 (ddd, J=17.2, 4.5, 2.2 Hz, 1H), 2.39 (dddd, J=30.8, 15.9, 8.2, 4.5 Hz, 5H), 2.19 (td, J=14.4, 5.9 Hz, 2H), 2.05-1.93 (m, 2H), 1.88 (d, J=12.4 Hz, 2H), 1.78-1.70 (m, 2H), 1.46 (qt, J=12.4, 3.5 Hz, 2H), 1.24 (s, 1H), 1.05 (dd, J=14.3, 10.0 Hz, 2H).

Compound 650A: LCMS: 557.15 (M+H). Method: Mobile Phase A: Water/5 mM NH₄HCO₃. Mobile Phase B: Acetonitrile. Flow rate: 1.5 mL/min. COLUMN: HPH-C18 (50×3.0) mm, 4.0μ. Rt (min): 1.838; Area %—97.67. ¹H NMR (400 MHz, DMSO-d6): δ 10.97 (s, 1H), 7.48-7.40 (m, 1H), 7.37 (dd, J=7.4, 2.3 Hz, 2H), 5.08 (dd, J=13.2, 5.2 Hz, 1H), 4.32 (d, J=17.3 Hz, 1H), 4.23 (d, J=17.3 Hz, 1H), 3.22 (t, J=6.3 Hz, 3H), 3.01-2.83 (m, 2H), 2.77-2.68 (m, 2H), 2.65-2.51 (m, 2H), 2.49-2.34 (m, 4H), 2.22-2.06 (m, 2H), 2.02 (dtd, J=11.9, 6.8, 6.0, 3.4 Hz, 2H), 1.79 (d, J=13.3 Hz, 1H), 1.71 (t, J=12.5 Hz, 3H), 1.51-1.34 (m, 4H), 1.24 (s, 1H).

### Compounds 651 and 651A

### 3-(1-oxo-4-{[(1r,4r)-4-[(3,3,3-trifluoropropyl)amino]cyclohexyl]({spiro[3.3]heptan-2-ylmethyl})amino}-3H-isoindol-2-yl)piperidine-2,6-dione; trifluoroacetic acid and 3-(1-oxo-4-{[(1s,4s)-4-[(3,3,3-trifluoropropyl)amino]cyclohexyl]({spiro[3.3]heptan-2-ylmethyl})amino}-3H-isoindol-2-yl)piperidine-2,6-dione; bis(trifluoroacetic acid)

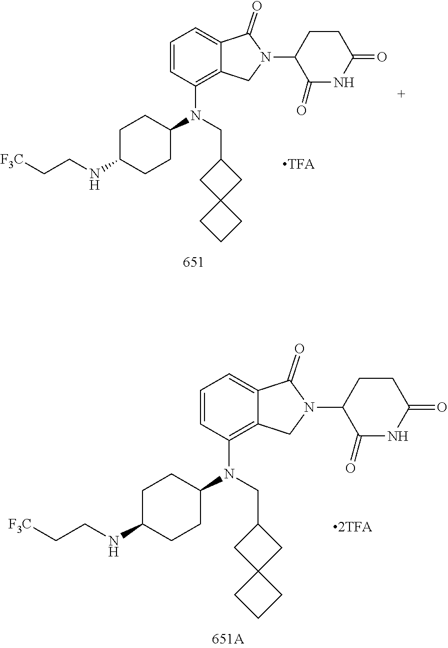 [SMILES: O=C1CCC(N2Cc3c(cccc3N(CC3CC4(CCC4)C3)[C@H]3CC[C@H](NCCC(F)(F)F)CC3)C2=O)C(=O)N1 ; O=C1CCC(N2Cc3c(cccc3N(CC3CC4(CCC4)C3)[C@H]3CC[C@@H](NCCC(F)(F)F)CC3)C2=O)C(=O)N1]

The compound was prepared by the same procedure described as preparation of Compounds 554 and 554A above.

Compound 651: LCMS: 561.45 (M+H). Method: Mobile Phase A: Water/0.04% NH₃H₂O. Mobile Phase B: Acetonitrile. Flow rate: 1.5 mL/min. COLUMN: EVO-C18 (50×3.0) mm, 2.6μ. Rt (min): 1.432; Area %—97.82. ¹H NMR (400 MHz, DMSO-d6): δ 10.97 (s, 1H), 8.75 (q, J=6.5, 6.0 Hz, 2H), 7.43 (t, J=7.6 Hz, 1H), 7.34 (dd, J=15.9, 7.6 Hz, 2H), 5.12 (dd, J=13.2, 5.1 Hz, 1H), 4.29 (d, J=17.3 Hz, 1H), 4.19 (d, J=17.3 Hz, 1H), 3.18 (q, J=6.9 Hz, 2H), 2.99 (dddd, J=53.2, 17.9, 13.4, 5.8 Hz, 5H), 2.76-2.69 (m, 1H), 2.67 (dd, J=10.0, 6.1 Hz, 1H), 2.64-2.55 (m, 1H), 2.50-2.44 (m, 2H), 2.08 (d, J=3.3 Hz, 1H), 2.05 (s, 1H), 2.03-1.93 (m, 2H), 1.93-1.79 (m, 8H), 1.76-1.63 (m, 2H), 1.52 (dtd, J=20.9, 10.6, 8.8, 4.9 Hz, 4H), 1.42-1.28 (m, 2H). ¹⁹F NMR (377 MHz, DMSO-d6): δ −64.07 (s,1), −74.11 (s,1).

Compound 651A: LCMS: 561.40 (M+H). Method: Mobile Phase A: Water/0.04% NH₃H₂O. Mobile Phase B: Acetonitrile. Flow rate: 1.5 mL/min. COLUMN: EVO-C18 (50×3.0) mm, 2.6μ. Rt (min): 1.471; Area % —99.52. ¹H NMR (400 MHz, DMSO-d6): δ 10.99 (s, 1H), 8.78 (t, J=6.4 Hz, 2H), 7.48-7.33 (m, 3H), 5.14-5.10 (m, 1H), 4.36 (d, J=17.3 Hz, 1H), 4.25 (d, J=17.3 Hz, 1H), 3.24 (dtd, J=16.5, 11.6, 11.5, 7.4 Hz, 4H), 3.10 (pd, J=15.7, 13.6, 5.1 Hz, 2H), 2.92 (dt, J=18.2, 7.0 Hz, 1H), 2.83-2.66 (m, 2H), 2.61 (dt, J=17.1, 3.5 Hz, 1H), 2.50-2.40 (m, 2H), 2.10-1.95 (m, 2H), 1.95-1.56 (m, 13H), 1.56-1.43 (m, 3H). ¹⁹F NMR (377 MHz, DMSO-d6): δ −64.10 (s,1), −74.55 (s,2).

### Compounds 652 and 652A

### 3-(4-{[(3,3-dimethylcyclobutyl)methyl][(1r,4r)-4-[(3,3,3-trifluoropropyl)amino]cyclohexyl]amino}-1-oxo-3H-isoindol-2-yl)piperidine-2,6-dione; bis(trifluoroacetic acid) and 3-(4-{[(3,3-dimethylcyclobutyl)methyl][(1s,4s)-4-[(3,3,3-trifluoropropyl)amino]cyclohexyl]amino}-1-oxo-3H-isoindol-2-yl)piperidine-2,6-dione; bis(trifluoroacetic acid)

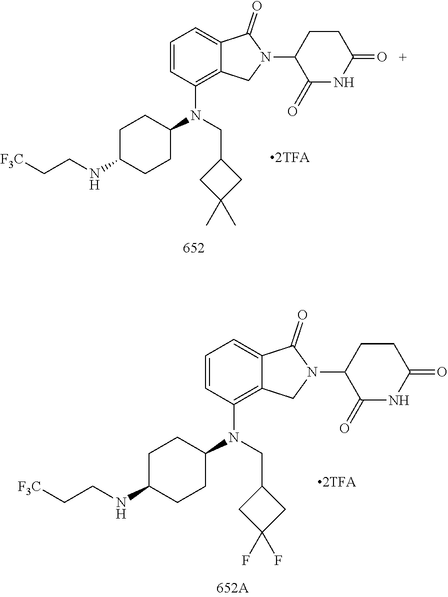 [SMILES: CC1(C)CC(CN(c2cccc3c2CN(C2CCC(=O)NC2=O)C3=O)[C@H]2CC[C@H](NCCC(F)(F)F)CC2)C1 ; O=C1CCC(N2Cc3c(cccc3N(CC3CC(F)(F)C3)[C@H]3CC[C@@H](NCCC(F)(F)F)CC3)C2=O)C(=O)N1]

The compound was prepared by the same procedure described as preparation of Compounds 554 and 554A above.

Compound 652: LCMS: 549.3 (M+H). Method: Mobile Phase A: Water/0.04% NH₃H₂O. Mobile Phase B: Acetonitrile. Flow rate: 1.5 mL/min. COLUMN: EVO-C18 (50×3.0) mm, 2.6μ. Rt (min): 1.531; Area %—99.31. ¹H NMR (400 MHz, DMSO-d6): δ 10.97 (s, 1H), 8.84 (d, J=7.7 Hz, 2H), 7.48-7.27 (m, 3H), 5.12 (dd, J=13.2, 5.1 Hz, 1H), 4.35-4.14 (m, 2H), 3.12 (ddt, J=24.6, 13.4, 7.1 Hz, 5H), 3.02-2.85 (m, 2H), 2.76-2.53 (m, 3H), 2.49-2.43 (m, 1H), 2.16-1.94 (m, 4H), 1.84 (d, J=12.1 Hz, 2H), 1.68-1.43 (m, 4H), 1.35 (t, J=9.5 Hz, 4H), 0.99 (d, J=8.5 Hz, 6H). ¹⁹F NMR (376 MHz, DMSO-d6): δ −64.12 (s,1), −74.64 (t, J=4.3 Hz,2).

Compound 652A: LCMS: 549.3 (M+H). Method: Mobile Phase A: Water/0.04% NH₃H₂O. Mobile Phase B: Acetonitrile. Flow rate: 1.5 mL/min. COLUMN: EVO-C18 (50×3.0) mm, 2.6μ. Rt (min): 1.609; Area %—98.90. ¹H NMR (400 MHz, DMSO-d6): δ 10.98 (s, 1H), 8.79 (s, 2H), 7.50-7.32 (m, 3H), 5.11 (dd, J=13.2, 5.1 Hz, 1H), 4.36 (d, J=17.4 Hz, 1H), 4.31 (s, 1H), 3.34-3.17 (m, 4H), 3.10 (td, J=13.6, 12.2, 7.2 Hz, 2H), 2.92 (ddd, J=17.9, 13.5, 5.4 Hz, 1H), 2.82-2.66 (m, 2H), 2.65-2.54 (m, 1H), 2.44 (dd, J=13.2, 4.5 Hz, 1H), 2.15 (hept, J=8.1 Hz, 1H), 2.03 (ddd, J=11.5, 9.2, 6.2 Hz, 1H), 1.95-1.74 (m, 4H), 1.68 (q, J=8.2 Hz, 2H), 1.57 (qd, J=11.4, 9.7, 5.7 Hz, 4H), 1.30 (ddd, J=12.3, 8.4, 4.4 Hz, 2H), 0.97 (d, J=9.1 Hz, 6H). ¹⁹F NMR (376 MHz, DMSO-d6): δ −64.12 (s,1), −74.59-−75.05 (m, 2).

### Compounds 653 and 653A

### 3-(4-((cyclopropylmethyl)((1r,4r)-4-(((1-(trifluoromethyl)cyclopropyl)methyl)amino)cyclohexyl)amino)-1-oxoisoindolin-2-yl)piperidine-2,6-dione formate and 3-(4-((cyclopropylmethyl)((1s,4s)-4-(((1-(trifluoromethyl)cyclopropyl)methyl)amino)cyclohexyl)amino)-1-oxoisoindolin-2-yl)piperidine-2,6-dione bis(2,2,2-trifluoroacetate)

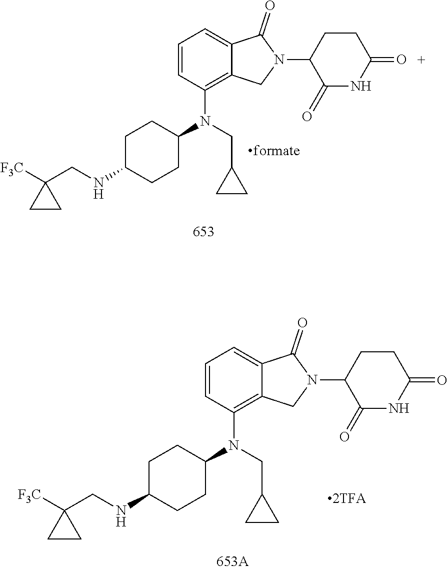 [SMILES: O=C1CCC(N2Cc3c(cccc3N(CC3CC3)[C@H]3CC[C@H](NCC4(C(F)(F)F)CC4)CC3)C2=O)C(=O)N1 ; O=C1CCC(N2Cc3c(cccc3N(CC3CC3)[C@H]3CC[C@@H](NCC4(C(F)(F)F)CC4)CC3)C2=O)C(=O)N1]

The compound was prepared by the same procedure described as preparation of Compounds 554 and 554A above.

Compound 653: LCMS: 533.25 (M+H) Method: Mobile phase A: Water/0.1% FA, Mobile phase B: Acetonitrile/0.1% FA. Flow rate: 1.5000 mL/min Column Name: HPH (50×3.0) mm, 4.0 μm. Rt (min): 0.849; Area %—99.530. ¹H NMR (400 MHz, DMSO-d₆): ¹H NMR (400 MHz, DMSO-d6) δ 10.96 (s, 1H), 8.16 (s, 1H), 7.42 (t, J=7.6 Hz, 1H), 7.34 (dd, J=13.9, 7.5 Hz, 2H), 5.09 (dd, J=13.1, 5.1 Hz, 1H), 4.32 (q, J=17.2 Hz, 2H), 3.07-2.86 (m, 4H), 2.76 (s, 2H), 2.64-2.54 (m, 2H), 2.25 (td, J=10.7, 5.3 Hz, 1H), 2.01 (ddd, J=11.8, 6.2, 3.9 Hz, 1H), 1.89-1.79 (m, 2H), 1.78-1.70 (m, 2H), 1.36 (d, J=12.5 Hz, 2H), 1.02 (q, J=12.7 Hz, 2H), 0.87-0.77 (m, 4H), 0.69 (td, J=7.2, 3.4 Hz, 1H), 0.29 (dq, J=8.5, 3.2 Hz, 2H).

Compound 653A: LCMS: 533.20 (M+H) Method: Mobile phase A: Water/0.1% FA, Mobile phase B: Acetonitrile/0.1% FA. Flow rate: 1.5000 mL/min Column Name: HPH (50×3.0) mm, 4.0 μm. Rt (min): 0.970; Area %—98.783. ¹H NMR (400 MHz, DMSO-d₆): ¹H NMR (400 MHz, DMSO-d6) δ 11.00 (s, 1H), 8.56 (s, 2H), 7.53-7.44 (m, 3H), 5.14 (dd, J=13.3, 5.1 Hz, 1H), 4.47-4.31 (m, 2H), 3.55 (s, 1H), 3.26 (t, J=5.9 Hz, 2H), 3.12 (d, J=9.3 Hz, 1H), 2.99-2.88 (m, 3H), 2.66-2.57 (m, 1H), 2.40 (td, J=13.2, 4.5 Hz, 1H), 2.10-2.01 (m, 1H), 1.87 (d, J=14.0 Hz, 2H), 1.70 (d, J=6.1 Hz, 4H), 1.48 (s, 2H), 1.18-1.05 (m, 4H), 0.76-0.67 (m, 1H), 0.29 (dt, J=8.0, 1.7 Hz, 2H), −0.06-−0.20 (m, 2H). ¹⁹F NMR (377 MHz, DMSO-d6): δ −67.64 (d, J=5.5 Hz,1), −74.55 (d, J=24.9 Hz,2).

### Compounds 654 and 654A

### 3-(4-((cyclobutylmethyl)((1r,4r)-4-(((1-(trifluoromethyl)cyclopropyl)methyl)amino)cyclohexyl)amino)-1-oxoisoindolin-2-yl)piperidine-2,6-dione and 3-(4-((cyclobutylmethyl)((1s,4s)-4-(((1-(trifluoromethyl)cyclopropyl)methyl)amino)cyclohexyl)amino)-1-oxoisoindolin-2-yl)piperidine-2,6-dione bis(2,2,2-trifluoroacetate)

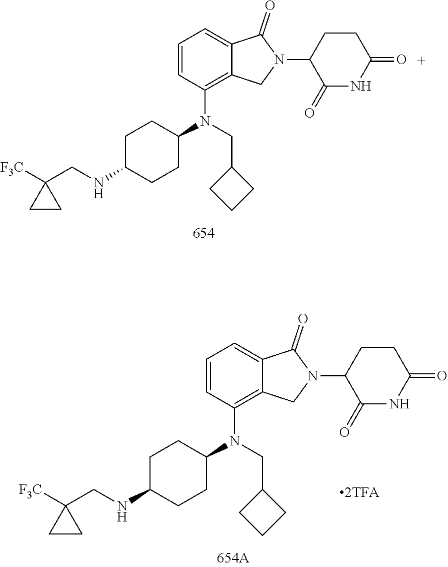 [SMILES: O=C1CCC(N2Cc3c(cccc3N(CC3CCC3)[C@H]3CC[C@H](NCC4(C(F)(F)F)CC4)CC3)C2=O)C(=O)N1 ; O=C1CCC(N2Cc3c(cccc3N(CC3CCC3)[C@H]3CC[C@@H](NCC4(C(F)(F)F)CC4)CC3)C2=O)C(=O)N1]

The compound was prepared by the same procedure described as preparation of Compounds 554 and 554A above.

Compound 654: LCMS: 547.25 (M+H) Method: Mobile phase A:Water/0.1% FA, Mobile phase B: Acetonitrile/0.1% FA. Flow rate: 1.8000 mL/min Column Name: HPH (50×3.0) mm, 4 μm. Rt (min): 1.032; Area %—98.565. ¹H NMR (400 MHz, DMSO-d₆): ¹H NMR (400 MHz, DMSO-d6) δ 10.95 (s, 1H), 7.40 (t, J=7.6 Hz, 1H), 7.28 (dd, J=17.6, 7.6 Hz, 2H), 5.08 (dd, J=13.2, 5.2 Hz, 1H), 4.24 (d, J=17.8 Hz, 2H), 3.10 (p, J=6.2 Hz, 2H), 2.96-2.84 (m, 2H), 2.77 (s, 2H), 2.61 (s, 1H), 2.28 (s, 1H), 2.13 (q, J=7.5 Hz, 1H), 2.04-1.96 (m, 1H), 1.86 (d, J=12.4 Hz, 2H), 1.81-1.66 (m, 6H), 1.56 (q, J=8.4, 7.9 Hz, 2H), 1.45 (t, J=11.4 Hz, 2H), 1.02 (d, J=13.6 Hz, 2H), 0.80 (dd, J=5.8, 4.0 Hz, 4H).

Compound 654A: LCMS: 547.20 (M+H) Method: Mobile phase A: Water/0.1% FA, Mobile phase B: Acetonitrile/0.1% FA. Flow rate: 1.8000 mL/min Column Name: HPH (50×3.0) mm, 4 μm. Rt (min): 1.043; Area %—99.400. ¹H NMR (400 MHz, DMSO-d₆): ¹H NMR (400 MHz, DMSO-d6) δ 11.00 (s, 1H), 8.55 (s, 2H), 7.49-7.37 (m, 3H), 5.13 (dd, J=13.2, 5.1 Hz, 1H), 4.34 (d, J=26.2 Hz, 2H), 3.35-3.24 (m, 3H), 3.14-3.04 (m, 3H), 2.94 (ddd, J=17.2, 13.6, 5.4 Hz, 1H), 2.66-2.57 (m, 1H), 2.42 (td, J=13.2, 4.6 Hz, 1H), 2.24 (h, J=7.5 Hz, 1H), 2.04 (ddq, J=10.7, 5.6, 3.4, 2.8 Hz, 1H), 1.86-1.63 (m, 10H), 1.58-1.43 (m, 4H), 1.20-1.11 (m, 2H), 1.09 (s, 2H). ¹⁹F NMR (377 MHz, DMSO-d6): δ −67.65 (s,1), −74.40 (s,2).

### Compounds 655 and 655A

### 3-(4-{[(3,3-difluorocyclobutyl)methyl][(1r,4r)-4-({[1-(trifluoromethyl)cyclopropyl]methyl}amino)cyclohexyl]amino}-1-oxo-3H-isoindol-2-yl)piperidine-2,6-dione and 3-(4-{[(3,3-difluorocyclobutyl)methyl][(1s,4s)-4-({[1-(trifluoromethyl)cyclopropyl]methyl}amino)cyclohexyl]amino}-1-oxo-3H-isoindol-2-yl)piperidine-2,6-dione

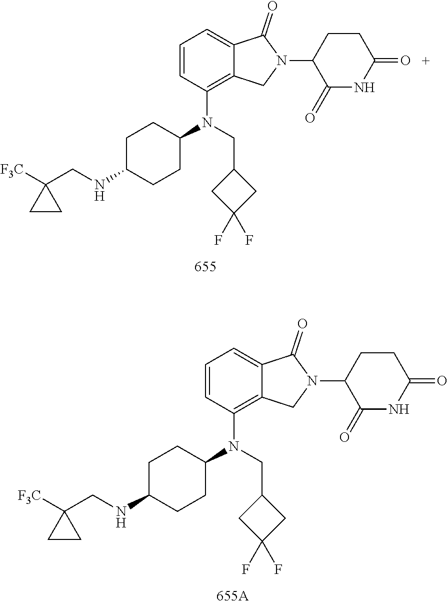 [SMILES: O=C1CCC(N2Cc3c(cccc3N(CC3CC(F)(F)C3)[C@H]3CC[C@H](NCC4(C(F)(F)F)CC4)CC3)C2=O)C(=O)N1 ; O=C1CCC(N2Cc3c(cccc3N(CC3CC(F)(F)C3)[C@H]3CC[C@@H](NCC4(C(F)(F)F)CC4)CC3)C2=O)C(=O)N1]

The compound was prepared by the same procedure described as preparation of Compounds 554 and 554A above.

Compound 655: LC-MS: (ES, m/z): [M+1]=583.30. 1H NMR (400 MHz, DMSO-d6) δ 10.96 (s, 1H), 7.48-7.39 (m, 1H), 7.36 (d, J=6.9 Hz, 2H), 5.08 (dd, J=13.2, 5.1 Hz, 1H), 4.31 (d, J=17.4 Hz, 2H), 3.19 (dd, J=7.8, 3.2 Hz, 2H), 2.92 (t, J=6.6 Hz, 2H), 2.90-2.83 (m, 2H), 2.74 (d, J=13.9 Hz, 1H), 2.65-2.52 (m, 2H), 2.43 (ddd, J=13.3, 10.9, 6.4 Hz, 3H), 2.25-2.21 (m, 2H), 2.15 (td, J=14.3, 13.8, 5.3 Hz, 2H), 2.06-1.92 (m, 2H), 1.86 (d, J=12.3 Hz, 3H), 0.89-0.80 (m, 2H), 0.80-0.73 (m, 4H).

Compound 655A: LC-MS: (ES, m/z): [M+1]=583.35. ¹H NMR (400 MHz, DMSO-d₆) δ 10.97 (s, 1H), 7.47-7.39 (m, 3H), 5.08 (dd, J=13.2, 5.1 Hz, 1H), 4.32 (d, J=17.4 Hz, 2H), 3.22 (dd, J=7.5, 4.3 Hz, 2H), 3.03-2.83 (m, 2H), 2.72 (s, 2H), 2.65-2.54 (m, 2H), 2.54-2.46 (m, 2H), 2.46-2.39 (m, 2H), 2.22-2.05 (m, 2H), 2.01 (qd, J=7.7, 6.7, 3.5 Hz, 5H), 1.81 (dd, J=12.1, 3.5 Hz, 4H), 0.89-0.80 (m, 4H).

### Compounds 656 and 656A

### 3-(1-oxo-4-{[(1r,4r)-4-({[1-(trifluoromethyl)cyclopropyl]methyl}amino)cyclohexyl]({spiro[3.3]heptan-2-ylmethyl})amino}-3H-isoindol-2-yl)piperidine-2,6-dione; trifluoroacetic acid and 3-(1-oxo-4-{[(1s,4s)-4-({[1-(trifluoromethyl)cyclopropyl]methyl}amino)cyclohexyl]({spiro[3.3]heptan-2-ylmethyl})amino}-3H-isoindol-2-yl)piperidine-2,6-dione; trifluoroacetic acid

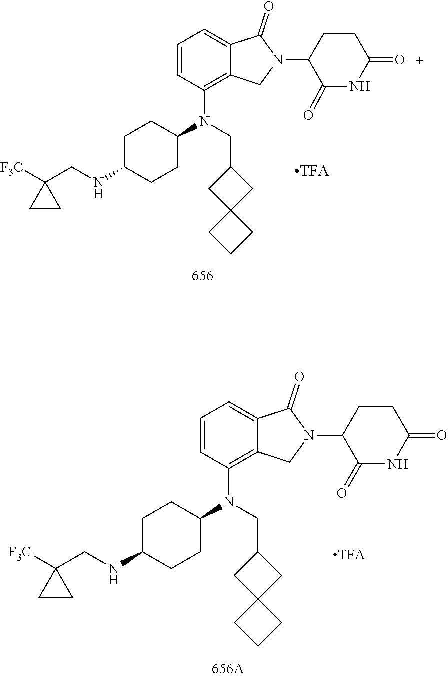 [SMILES: O=C1CCC(N2Cc3c(cccc3N(CC3CC4(CCC4)C3)[C@H]3CC[C@H](NCC4(C(F)(F)F)CC4)CC3)C2=O)C(=O)N1 ; O=C1CCC(N2Cc3c(cccc3N(CC3CC4(CCC4)C3)[C@H]3CC[C@@H](NCC4(C(F)(F)F)CC4)CC3)C2=O)C(=O)N1]

The compound was prepared by the same procedure described as preparation of Compounds 554 and 554A above.

Compound 656: LCMS:587.45 (M+H). Method: Mobile Phase A: Water/0.04% NH₃H₂O. Mobile Phase B: Acetonitrile. Flow rate: 1.5 mL/min. COLUMN: EVO-C18 (50×3.0) mm, 2.6μ. Rt (min): 1.650; Area %—95.89. ¹H NMR (400 MHz, DMSO-d6): δ 10.97 (s, 1H), 8.64 (d, J=7.5 Hz, 2H), 7.43 (t, J=7.6 Hz, 1H), 7.33 (dd, J=17.7, 7.5 Hz, 2H), 5.12 (dd, J=13.2, 5.1 Hz, 1H), 4.28 (d, J=17.3 Hz, 1H), 4.19 (d, J=17.3 Hz, 1H), 3.24 (t, J=5.8 Hz, 2H), 3.05 (dt, J=10.5, 5.2 Hz, 2H), 3.02-2.98 (m, 1H), 2.91 (dq, J=13.6, 6.9, 5.8 Hz, 2H), 2.66-2.53 (m, 1H), 2.50-2.42 (m, 2H), 2.11-2.04 (m, 2H), 2.04-1.91 (m, 2H), 1.83 (td, J=7.7, 4.8 Hz, 7H), 1.75-1.63 (m, 2H), 1.53 (ddd, J=11.1, 7.6, 2.6 Hz, 2H), 1.49-1.43 (m, 2H), 1.43-1.32 (m, 2H), 1.24 (s, 1H), 1.12 (d, J=4.9 Hz, 2H), 1.06 (d, J=4.7 Hz, 2H). ¹⁹F NMR (377 MHz, DMSO-d6): δ −67.81 (s,1), −74.03 (s,1).

Compound 656A: LCMS:587.45 (M+H). Method: Mobile Phase A: Water/0.04% NH₃H₂O. Mobile Phase B: Acetonitrile. Flow rate: 1.5 mL/min. COLUMN: EVO-C18 (50×3.0) mm, 2.6μ. Rt (min): 1.731; Area %—99.74. ¹H NMR (400 MHz, DMSO-d6): δ 11.00 (s, 1H), 8.72 (s, 2H), 7.60-7.28 (m, 3H), 5.13 (dd, J=13.2, 5.2 Hz, 1H), 4.39 (d, J=17.4 Hz, 1H), 4.29 (d, J=17.3 Hz, 1H), 3.31 (dt, J=11.1, 4.0 Hz, 3H), 3.14 (s, 1H), 3.09-3.01 (m, 2H), 2.94 (ddd, J=17.2, 13.6, 5.4 Hz, 1H), 2.67-2.52 (m, 1H), 2.45 (td, J=13.2, 4.5 Hz, 2H), 2.15-1.98 (m, 2H), 1.85 (q, J=6.1, 4.6 Hz, 5H), 1.81-1.75 (m, 3H), 1.75-1.61 (m, 5H), 1.52-1.37 (m, 4H), 1.14 (d, J=4.9 Hz, 2H), 1.13-1.05 (m, 2H). ¹⁹F NMR (377 MHz, DMSO-d₆): δ −67.79 (s,1), −74.52 (s,1).

### Compounds 657 and 657A

### 3-(4-{[(3,3-dimethylcyclobutyl)methyl][(1r,4r)-4-({[1-(trifluoromethyl)cyclopropyl]methyl}amino)cyclohexyl]amino}-1-oxo-3H-isoindol-2-yl)piperidine-2,6-dione; bis(trifluoroacetic acid) and 3-(4-{[(3,3-dimethylcyclobutyl)methyl][(1s,4s)-4-({[1-(trifluoromethyl)cyclopropyl]methyl}amino)cyclohexyl]amino}-1-oxo-3H-isoindol-2-yl)piperidine-2,6-dione; bis(trifluoroacetic acid)

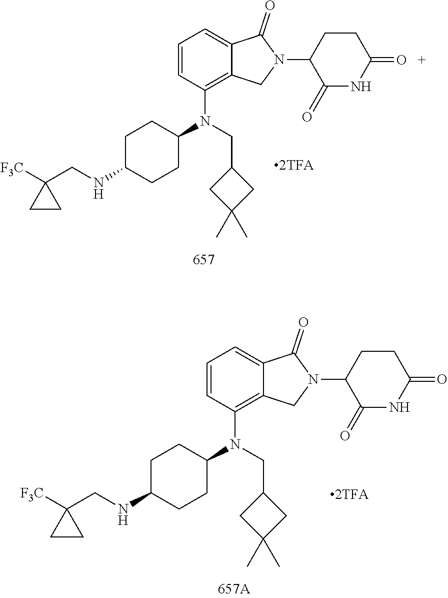 [SMILES: CC1(C)CC(CN(c2cccc3c2CN(C2CCC(=O)NC2=O)C3=O)[C@H]2CC[C@H](NCC3(C(F)(F)F)CC3)CC2)C1 ; CC1(C)CC(CN(c2cccc3c2CN(C2CCC(=O)NC2=O)C3=O)[C@H]2CC[C@@H](NCC3(C(F)(F)F)CC3)CC2)C1]

The compound was prepared by the same procedure described as preparation of Compounds 554 and 554A above.

Compound 657: LCMS: 575.2 (M+H). Method: Mobile Phase A: Water/5 mM NH₄HCO₃. Mobile Phase B: Acetonitrile. Flow rate: 1.5 mL/min. COLUMN: HPH-C18 (50×3.0) mm, 4.0μ. Rt (min): 1.666; Area %—92.29. ¹H NMR (400 MHz, DMSO-d6): δ 10.97 (s, 1H), 8.69 (s, 2H), 7.51-7.22 (m, 3H), 5.12 (dd, J=13.2, 5.1 Hz, 1H), 4.24 (q, J=17.3 Hz, 2H), 3.25 (t, J=5.9 Hz, 2H), 3.11 (t, J=6.7 Hz, 2H), 3.05-2.82 (m, 3H), 2.65-2.54 (m, 1H), 2.46 (d, J=8.4 Hz, 1H), 2.15-1.94 (m, 4H), 1.83 (d, J=11.6 Hz, 2H), 1.59 (q, J=9.1 Hz, 2H), 1.40 (dt, J=37.2, 8.2 Hz, 6H), 1.12 (d, J=4.9 Hz, 2H), 1.07 (d, J=6.3 Hz, 2H), 0.99 (d, J=8.7 Hz, 6H). ¹⁹F NMR (376 MHz, DMSO-d6): δ −67.86 (s,1), −74.57 (s,2).

Compound 657A: LCMS: 575.2 (M+H). Method: Mobile Phase A: Water/5 mM NH₄HCO₃. Mobile Phase B: Acetonitrile. Flow rate: 1.5 mL/min. COLUMN: HPH-C18 (50×3.0) mm, 4.0μ. Rt (min): 1.742; Area %—99.01. ¹H NMR (400 MHz, DMSO-d6): δ 11.00 (s, 1H), 8.68 (s, 2H), 7.51-7.35 (m, 3H), 5.13 (dd, J=13.3, 5.1 Hz, 1H), 4.46-4.24 (m, 2H), 3.31 (dt, J=11.1, 5.3 Hz, 3H), 3.09 (ddd, J=21.0, 16.1, 10.9 Hz, 3H), 2.94 (ddd, J=17.1, 13.6, 5.4 Hz, 1H), 2.66-2.56 (m, 1H), 2.49-2.38 (m, 1H), 2.20 (p, J=7.9 Hz, 1H), 2.09-1.99 (m, 1H), 1.92-1.73 (m, 4H), 1.69 (dt, J=11.2, 5.5 Hz, 2H), 1.57 (td, J=8.0, 4.0 Hz, 2H), 1.52-1.40 (m, 2H), 1.25 (td, J=12.3, 11.8, 8.4 Hz, 3H), 1.14 (d, J=4.8 Hz, 2H), 1.09 (s, 2H), 0.99 (s, 3H), 0.94 (s, 3H). ¹⁹F NMR (376 MHz, DMSO-d6): δ −67.57-−67.96 (m, 1), −74.56-−75.07 (m, 2).

### Compounds 658 and 658A

### 3-{4-[(2-cyclopropylethyl)[(1r,4r)-4-({[1-(trifluoromethyl)cyclopropyl]methyl}amino)cyclohexyl]amino]-6-fluoro-1-oxo-3H-isoindol-2-yl}piperidine-2,6-dione and 3-{4-[(2-cyclopropylethyl)[(1s,4s)-4-({[1-(trifluoromethyl)cyclopropyl]methyl}amino)cyclohexyl]amino]-6-fluoro-1-oxo-3H-isoindol-2-yl}piperidine-2,6-dione

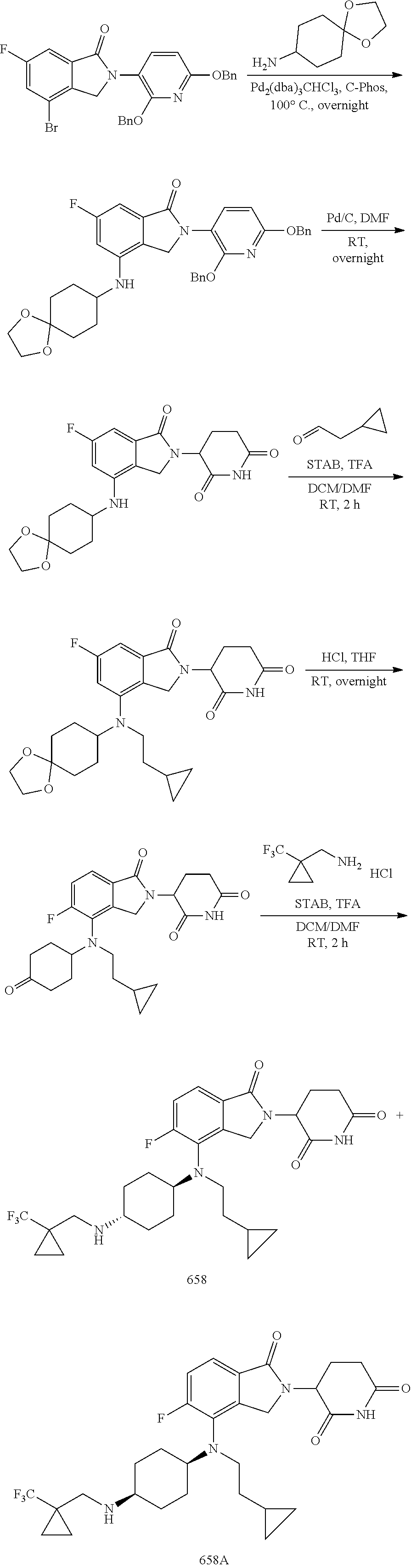

Step-1: Synthesis of 2-[2,6-bis(benzyloxy)pyridin-3-yl]-4-{1,4-dioxaspiro[4.5]decan-8-ylamnino}-6-fluoro-3H-isoindol-1-one

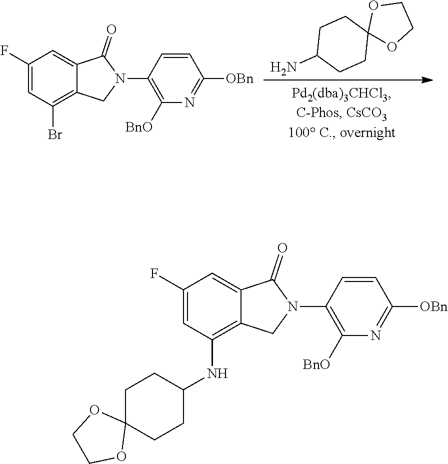 [SMILES: O=C1c2cc(F)cc(Br)c2CN1c1ccc(OCc2ccccc2)nc1OCc1ccccc1 ; NC1CCC2(CC1)OCCO2 ; O=C1c2cc(F)cc(NC3CCC4(CC3)OCCO4)c2CN1c1ccc(OCc2ccccc2)nc1OCc1ccccc1]

A solution of 2-[2,6-bis(benzyloxy)pyridin-3-yl]-4-bromno-6-fluoro-3H-isoindol-1-one (1.8 g, 3.466 mmol, 1 equiv) in dioxane (40 mL) was treated with 1,4-dioxaspiro[4.5]decan-8-amine (817 mg, 5.2 mmol, 1.5 equiv) and Cs₂CO₃ (4.5 g, 13.9 mmol, 4 equiv) for 5 min at room temperature under nitrogen atmosphere. To the above mixture was added CPhos (303 mg, 0.69 mmol, 0.2 equiv) and Pd₂(dba)₃CHCl₃ (359 mg, 0.35 mmol, 0.1 equiv) at room temperature. The resulting mixture was stirred for additional overnight at 100° C. The resulting mixture was diluted with water (200 mL). The resulting mixture was extracted with EtOAc (3×100 mL). The combined organic layers were washed with brine (3×50 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 2-[2,6-bis(benzyloxy)pyridin-3-yl]-4-{1,4-dioxaspiro[4.5]decan-8-ylamino}-6-fluoro-3H-isoindol-1-one (990 mg, 48%) as a yellow oil. LC/MS: mass calcd. for C₃₅H₃₄FN₃O₅: 595.25 found: 596.30[mass+H]. RT: 1.46 min.

### Step-2: Synthesis of 3-(4-{1,4-dioxaspiro[4.5]decan-8-ylamino}-6-fluoro-1-oxo-3H-isoindol-2-yl)piperidine-2,6-dione

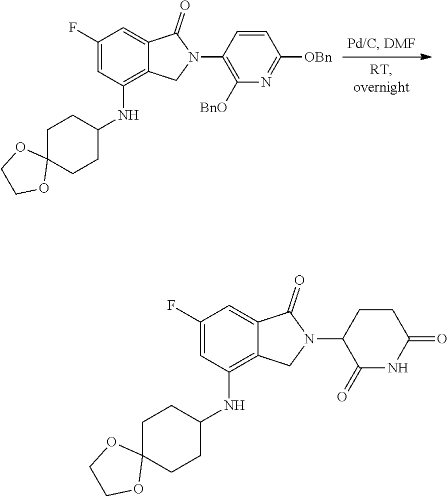 [SMILES: O=C1c2cc(F)cc(NC3CCC4(CC3)OCCO4)c2CN1c1ccc(OCc2ccccc2)nc1OCc1ccccc1 ; O=C1CCC(N2Cc3c(NC4CCC5(CC4)OCCO5)cc(F)cc3C2=O)C(=O)N1]

To a stirred solution of 2-[2,6-bis(benzyloxy)pyridin-3-yl]-4-{1,4-dioxaspiro[4.5]decan-8-ylamino}-6-fluoro-3H-isoindol-1-one (1.25 g, 2.1 mmol, 1 equiv) in DMF (10 mL) was added Palladium 10% on Carbon (wetted with ca. 55% Water) (1.25 g, 1.2 mmol, 0.56 equiv, 10%) at room temperature under nitrogen atmosphere. The resulting mixture was stirred for overnight at room temperature under hydrogen atmosphere. The resulting mixture was filtered, the filter cake was washed with THF (5×10 mL). The filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 3-(4-{1,4-dioxaspiro[4.5]decan-8-ylamino}-6-fluoro-1-oxo-3H-isoindol-2-yl)piperidine-2,6-dione (280 mg, 31.96%) as an off-white solid.

LC/MS: mass calcd. for C₂₁H₂₄FN₃O₅: 417.17 found: 418.05[mass+H]. RT: 0.93 min.

### Step-3: Synthesis of 3-{4-[(2-cyclopropylethyl)(1,4-dioxaspiro[4.5]decan-8-yl)amino]-6-fluoro-1-oxo-3H-isoindol-2-yl}piperidine-2,6-dione

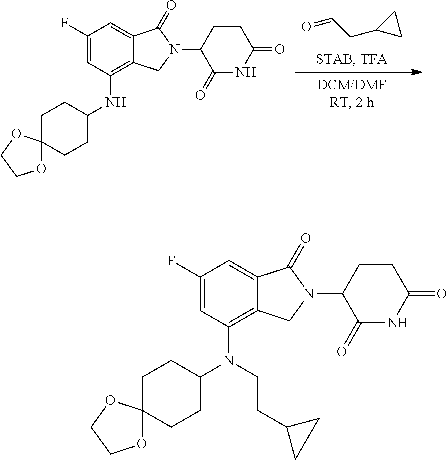

A solution of 3-(4-{1,4-dioxaspiro[4.5]decan-8-ylamino}-6-fluoro-1-oxo-3H-isoindol-2-yl)piperidine-2,6-dione (750 mg, 1.8 mmol, 1 equiv), 2-cyclopropylacetaldehyde (4.53 g, 53.9 mmol, 30 equiv) in DCM (18 mL) and DMF (4.5 mL) was treated with TFA (0.67 mL, 8.98 mmol, 5 equiv) for overnight at room temperature under nitrogen atmosphere followed by the addition of STAB (1.9 g, 8.98 mmol, 5 equiv) in portions at 0° C. The resulting mixture was stirred for additional 2 h at room temperature. The resulting mixture was diluted with water (100 mL) and extracted with EtOAc (3×50 mL). The combined organic layers were washed with brine (100 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 3-{4-[(2-cyclopropylethyl)(1,4-dioxaspiro[4.5]decan-8-yl)amino]-6-fluoro-1-oxo-3H-isoindol-2-yl}piperidine-2,6-dione (100 mg, 12%) as an off-white solid.

LC/MS: mass calcd. for C₂₆H₃₂FN₃O₅: 485.2 found: 486.2[mass+H]. RT: 1.07 min.

### Step-4: Synthesis of 3-{4-[(2-cyclopropylethyl)(4-oxocyclohexyl)amino]-6-fluoro-1-oxo-3H-isoindol-2-yl}piperidine-2,6-dione

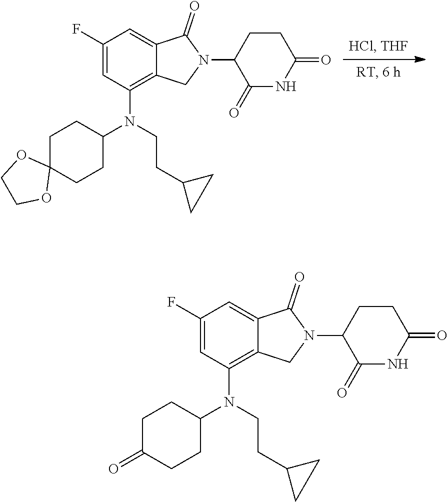 [SMILES: O=C1CCC(N2Cc3c(cc(F)cc3N(CCC3CC3)C3CCC4(CC3)OCCO4)C2=O)C(=O)N1 ; O=C1CCC(N(CCC2CC2)c2cc(F)cc3c2CN(C2CCC(=O)NC2=O)C3=O)CC1]

A solution of 3-{4-[(2-cyclopropylethyl)(1,4-dioxaspiro[4.5]decan-8-yl)amino]-6-fluoro-1-oxo-3H-isoindol-2-yl}piperidine-2,6-dione (200 mg, 0.41 mmol, 1 equiv) and HCl (6 mL, 2 M) in THF (3 mL) was stirred for 6 h at room temperature under nitrogen atmosphere. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 3-{4-[(2-cyclopropylethyl)(4-oxocyclohexyl)amino]-6-fluoro-1-oxo-3H-isoindol-2-yl}piperidine-2,6-dione (100 mg, 55%) as an off-white solid.

LC/MS: mass calcd. for C₂₄H₂₈FN₃O₄:441.2 found: 442.2[mass+H]. RT: 0.79 min.

### Step-5: Synthesis of 3-{4-[(2-cyclopropylethyl)[(1r,4r)-4-({[1-(trifluoromethyl)cyclopropyl]methyl}amino)cyclohexyl]amino]-6-fluoro-1-oxo-3H-isoindol-2-yl}piperidine-2,6-dione and 3-{4-[(2-cyclopropylethyl)[(1s,4s)-4-({[1-(trifluoromethyl)cyclopropyl]methyl}amino)cyclohexyl]amino]-6-fluoro-1-oxo-3H-isoindol-2-yl}piperidine-2,6-dione

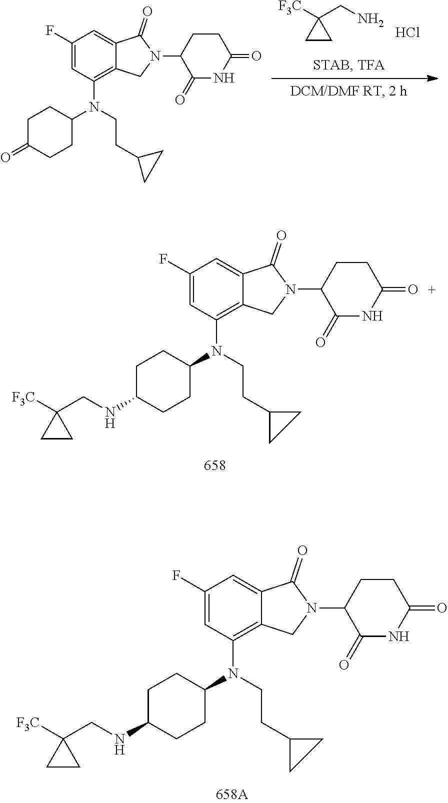 [SMILES: O=C1CCC(N(CCC2CC2)c2cc(F)cc3c2CN(C2CCC(=O)NC2=O)C3=O)CC1 ; NCC1(C(F)(F)F)CC1 ; Cl ; O=C1CCC(N2Cc3c(cc(F)cc3N(CCC3CC3)[C@H]3CC[C@H](NCC4(C(F)(F)F)CC4)CC3)C2=O)C(=O)N1 ; O=C1CCC(N2Cc3c(cc(F)cc3N(CCC3CC3)[C@H]3CC[C@@H](NCC4(C(F)(F)F)CC4)CC3)C2=O)C(=O)N1]

A solution of 3-{4-[(2-cyclopropylethyl)(4-oxocyclohexyl)amino]-6-fluoro-1-oxo-3H-isoindol-2-yl}piperidine-2,6-dione (100 mg, 0.226 mmol, 1 equiv), 1-[1-(trifluoromethyl)cyclopropyl]methanamine (63 mg, 0.45 mmol, 2 equiv) in DCM (0.3 mL), DMF (0.08 mL) was treated with TFA (129 mg, 1.13 mmol, 5 equiv) overnight at room temperature under nitrogen atmosphere followed by the addition of STAB (240 mg, 1.13 mmol, 5 equiv) in portions at 0° C. The resulting mixture was stirred for additional 2 h at room temperature. The resulting mixture was diluted with water and extracted with EtOAc (3×20 mL). The combined organic layers were washed with brine (2×100 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 3-{4-[(2-cyclopropylethyl)[(1r,4r)-4-({[1-(trifluoromethyl)cyclopropyl]methyl}amino)cyclohexyl]amino]-6-fluoro-1-oxo-3H-isoindol-2-yl}piperidine-2,6-dione (18.9 mg, 14.5%) (658) as an off-white solid and 3-{4-[(2-cyclopropylethyl)[(1s,4s)-4-({[1-(trifluoromethyl)cyclopropyl]methyl}amino)cyclohexyl]amino]-6-fluoro-1-oxo-3H-isoindol-2-yl}piperidine-2,6-dione (5.1 mg, 3.90%) (658A) as an off-white solid.

Compound 658: LCMS: 565.25 (M+H). Method: Mobile Phase A: Water/0.1% FA. Mobile Phase B: Acetonitrile/0.1% FA.Flow rate: 1.5 mL/min. COLUMN: Ascentis Express HALO-C18 (30×3.0) mm, 2μ. Rt (min): 1.085; Area %—99.684. ¹H NMR (400 MHz, DMSO-d6): δ 10.96 (s, 1H), 6.98-6.90 (m, 2H), 5.07 (dd, J=13.2, 5.1 Hz, 1H), 4.45-3.96 (m, 3H), 3.21 (d, J=7.9 Hz, 3H), 2.89 (ddd, J=18.0, 13.3, 5.4 Hz, 1H), 2.77 (s, 2H), 2.58 (dd, J=20.2, 6.0 Hz, 1H), 2.29 (s, 1H), 2.05-1.97 (m, 1H), 1.87 (d, J=12.2 Hz, 2H), 1.70 (s, 2H), 1.54 (s, 3H), 1.22-1.15 (m, 2H), 1.12-1.03 (m, 2H), 0.86-0.75 (m, 4H), 0.64 (d, J=5.5 Hz, 1H), 0.39-0.30 (m, 2H), −0.06 (d, J=4.1 Hz, 2H).

Compound 658A: LCMS: 565.25 (M+H). Method: Mobile Phase A: Water/0.1% FA. Mobile Phase B: Acetonitrile/0.1% FA. Flow rate: 1.5 mL/min. COLUMN: Ascentis Express HALO-C18 (30×3.0) mm, 2μ. Rt (min): 1.103; Area %—99.669. ¹H NMR (400 MHz, DMSO-d6):δ 10.96 (s, 1H), 6.92 (dd, J=9.4, 6.9 Hz, 2H), 5.08 (dd, J=13.2, 5.1 Hz, 1H), 4.41-4.18 (m, 2H), 3.25 (d, J=7.5 Hz, 2H), 2.89 (t, J=4.7 Hz, 1H), 2.72 (s, 2H), 2.63 (d, J=10.6 Hz, 2H), 2.05-1.97 (m, 1H), 1.89 (d, J=12.6 Hz, 2H), 1.72 (d, J=13.0 Hz, 2H), 1.59-1.38 (m, 5H), 1.22 (dd, J=9.7, 3.5 Hz, 3H), 0.85 (d, J=6.3 Hz, 4H), 0.64 (s, 1H), 0.40-0.31 (m, 2H).

### Compound 659

### 3-[(2-cyclopropylethyl)[2-(2,6-dioxopiperidin-3-yl)-1-oxo-3H-isoindol-4-yl]amino]bicyclo[1.1.1]pentane-1-carboxamide

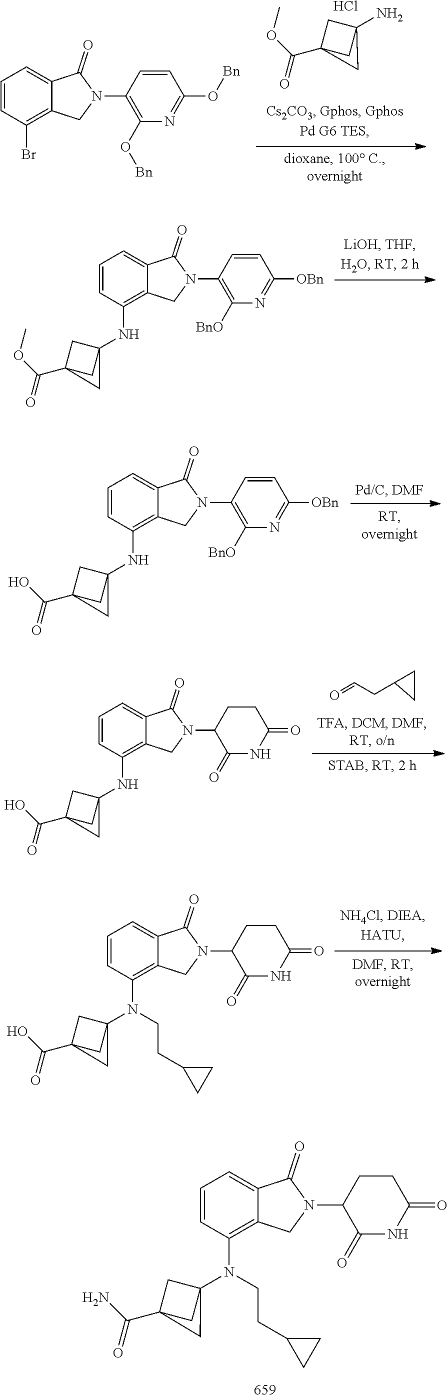

Step-1: Synthesis of methyl 3-({2-[2,6-bis(benzyloxy)pyridin-3-yl]-1-oxo-3H-isoindol-4-yl}amino)bicyclo[1.1.1]pentane-1-carboxylate

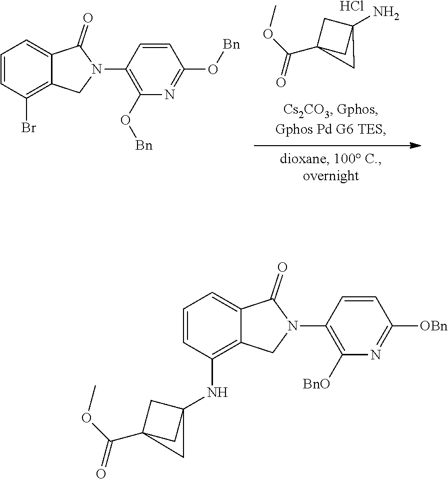

To a stirred solution of 2-[2,6-bis(benzyloxy)pyridin-3-yl]-4-bromo-3H-isoindol-1-one (1.0 g, 1.99 mmol, 1 equiv) and methyl 3-aminobicyclo[1.1.1]pentane-1-carboxylate hydrochloride (531 mg, 2.99 mmol, 1.5 equiv) in dioxane (20 mL) were added Cs₂CO₃ (1.9 g, 5.98 mmol, 3 equiv), Gphos (161 mg, 0.29 mmol, 0.15 equiv) and Gphos Pd G6 TES (188 mg, 0.19 mmol, 0.1 equiv) at room temperature under nitrogen atmosphere. The resulting mixture was stirred for overnight at 100° C. under nitrogen atmosphere. The resulting mixture was diluted with water (150 mL). The resulting mixture was extracted with EtOAc (3×300 mL). The combined organic layers were washed with brine (3×300 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 50% to 80% gradient in 8 min; detector, UV 254 nm to afford methyl 3-({2-[2,6-bis(benzyloxy)pyridin-3-yl]-1-oxo-3H-isoindol-4-yl}amino)bicyclo[1.1.1]pentane-1-carboxylate (360 mg, 32%) as a light yellow oil.

LC/MS: mass calcd. for C₃₄H₃₁N₃O₅: 561.3 found: 562.3[mass+H]. RT: 1.22 min.

### Step-2: Synthesis of 3-({2-[2,6-bis(benzyloxy)pyridin-3-yl]-1-oxo-3H-isoindol-4-yl}amino)bicyclo[1.1.1]pentane-1-carboxylic acid

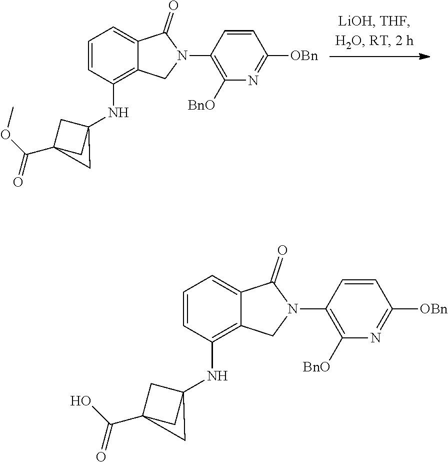 [SMILES: COC(=O)C12CC(Nc3cccc4c3CN(c3ccc(OCc5ccccc5)nc3OCc3ccccc3)C4=O)(C1)C2 ; O=C1c2cccc(NC34CC(C(=O)O)(C3)C4)c2CN1c1ccc(OCc2ccccc2)nc1OCc1ccccc1]

To a stirred solution of methyl 3-({2-[2,6-bis(benzyloxy)pyridin-3-yl]-1-oxo-3H-isoindol-4-yl}amino)bicyclo[1.1.1]pentane-1-carboxylate (360 mg, 0.64 mmol, 1 equiv) in THF (5 mL) and H₂O (1.75 mL) was added LiOH (77 mg, 3.2 mmol, 5 equiv) at room temperature. The resulting mixture was stirred for 2 h at room temperature. The mixture was acidified to pH 5 with 2M HCl (aq.). The resulting mixture was extracted with EtOAc (3×50 mL). The combined organic layers were washed with brine (3×150 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 20% to 50% gradient in 8 min; detector, UV 254 nm to afford 3-({2-[2,6-bis(benzyloxy)pyridin-3-yl]-1-oxo-3H-isoindol-4-yl}amino)bicyclo[1.1.1]pentane-1-carboxylic acid (250 mg, 71%) as a yellow solid.

LC/MS: mass calcd. for C₃₃H₂₉N₃O₅: 547.2 found: 548.2[mass+H]. RT: 1.12 min.

### Step-3: Synthesis of 3-{[2-(2,6-dioxopiperidin-3-yl)-1-oxo-3H-isoindol-4-yl]amino}bicyclo[1.1.1]pentane-1-carboxylic acid

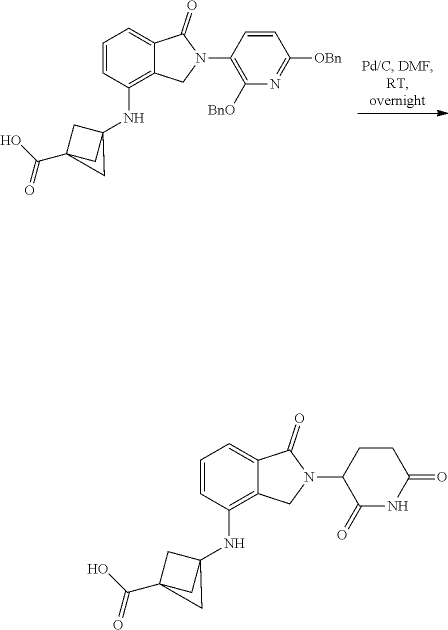 [SMILES: O=C1c2cccc(NC34CC(C(=O)O)(C3)C4)c2CN1c1ccc(OCc2ccccc2)nc1OCc1ccccc1 ; O=C1CCC(N2Cc3c(NC45CC(C(=O)O)(C4)C5)cccc3C2=O)C(=O)N1]

To a stirred solution of 3-({2-[2,6-bis(benzyloxy)pyridin-3-yl]-1-oxo-3H-isoindol-4-yl}amino)bicyclo[1.1.1]pentane-1-carboxylic acid (380 mg, 0.69 mmol, 1 equiv) in DMF (7 mL) was added Palladium 10% on Carbon (wetted with ca. 55% Water) (560 mg, 0.52 mmol, 0.76 equiv) at room temperature under nitrogen atmosphere. The resulting mixture was stirred for overnight at room temperature under hydrogen atmosphere. The resulting mixture was filtered, the filter cake was washed with THF (3×3 mL). The filtrate was concentrated under reduced pressure. The residue was purified by reversed-phase flash chromatography with the following conditions: column, C18 silica gel; mobile phase, MeCN in Water (0.1% FA), 10% to 40% gradient in 10 min; detector, UV 254 nm to afford 3-{[2-(2,6-dioxopiperidin-3-yl)-1-oxo-3H-isoindol-4-yl]amino}bicyclo[1.1.1]pentane-1-carboxylic acid (220 mg, 86%) as a pink solid.

LC/MS: mass calcd. for C₁₉H₁₉N₃O₅: 369.1 found: 370.1[mass+H]. RT: 0.62 min.

### Step-4: Synthesis of 3-[(2-cyclopropylethyl)[2-(2,6-dioxopiperidin-3-yl)-1-oxo-3H-isoindol-4-yl]amino]bicyclo[1.1.1]pentane-1-carboxylic acid

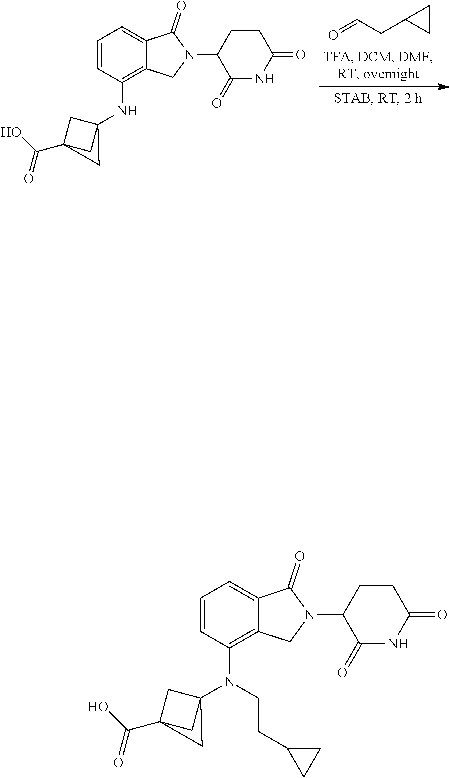

To a stirred solution of 3-{[2-(2,6-dioxopiperidin-3-yl)-1-oxo-3H-isoindol-4-yl]amino}bicyclo[1.1.1]pentane-1-carboxylic acid (200 mg, 0.54 mmol, 1 equiv) and 2-cyclopropylacetaldehyde (911 mg, 10.8 mmol, 20 equiv) in DCM (5 mL) and DMF (1.2 mL) was added TFA (185 mg, 1.6 mmol, 3 equiv) at room temperature. The resulting mixture was stirred for overnight at room temperature under nitrogen atmosphere. To the above mixture was added STAB (574 mg, 2.7 mmol, 5 equiv) in portions over 3 min at 0° C. The resulting mixture was stirred for additional 2 h at room temperature. The resulting mixture was diluted with water (100 mL). The resulting mixture was extracted with EtOAc (3×100 mL). The combined organic layers were washed with brine (3×200 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 20% to 40% gradient in 5 min; detector, UV 254 nm to afford 3-[(2-cyclopropylethyl)[2-(2,6-dioxopiperidin-3-yl)-1-oxo-3H-isoindol-4-yl]amino]bicyclo[1.1.1]pentane-1-carboxylic acid (220 mg, 93%) as a light yellow solid.

LC/MS: mass calcd. for C₂₄H₂₇N₃O₅: 437.1 found: 438.1[mass+H]. RT: 0.84 min.

### Step-5: Synthesis of 3-[(2-cyclopropylethyl)[2-(2,6-dioxopiperidin-3-yl)-1-oxo-3H-isoindol-4-yl]amino]bicyclo[1.1.1]pentane-1-carboxamide

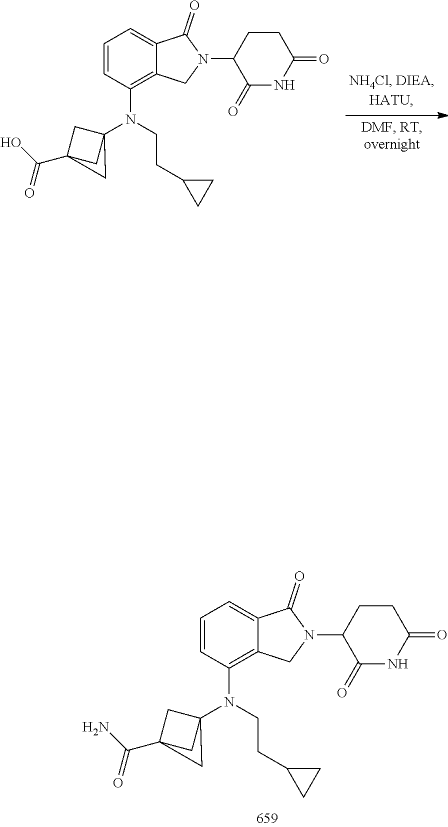 [SMILES: O=C1CCC(N2Cc3c(cccc3N(CCC3CC3)C34CC(C(=O)O)(C3)C4)C2=O)C(=O)N1 ; NC(=O)C12CC(N(CCC3CC3)c3cccc4c3CN(C3CCC(=O)NC3=O)C4=O)(C1)C2]

To a stirred solution of 3-[(2-cyclopropylethyl)[2-(2,6-dioxopiperidin-3-yl)-1-oxo-3H-isoindol-4-yl]amino]bicyclo[1.1.1]pentane-1-carboxylic acid (110 mg, 0.25 mmol, 1 equiv) and NH₄Cl (27 mg, 0.5 mmol, 2 equiv) in DMF (2 mL) were added DIEA (97 mg, 0.75 mmol, 3 equiv) and HATU (143 mg, 0.38 mmol, 1.5 equiv) at room temperature. The resulting mixture was stirred for overnight at room temperature. The resulting mixture was diluted with water (50 mL). The resulting mixture was extracted with EtOAc (3×100 mL). The combined organic layers were washed with brine (3×100 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 0% to 50% gradient in 8 min; detector, UV 254 nm to afford 3-[(2-cyclopropylethyl)[2-(2,6-dioxopiperidin-3-yl)-1-oxo-3H-isoindol-4-yl]amino]bicyclo[1.1.1]pentane-1-carboxamide (65 mg, 59%) as an off-white solid.

Compound 659: LCMS: 437.2 (M+H). Method: Mobile Phase A: Water/0.05% TFA. Mobile Phase B: Acetonitrile/0.05% TFA. Flow rate: 1.5 mL/min. COLUMN: EC-C18 (30×3.0) mm, 1.9μ. Rt (min): 1.739; Area %—98.47. ¹H NMR (400 MHz, DMSO-d6): δ 11.02 (d, J=2.9 Hz, 1H), 7.60-7.41 (m, 3H), 7.25 (s, 1H), 6.96 (s, 1H), 5.17 (dt, J=13.4, 4.1 Hz, 1H), 4.36 (qd, J=17.4, 3.0 Hz, 2H), 3.23 (td, J=7.5, 2.8 Hz, 2H), 3.06-2.88 (m, 1H), 2.69-2.59 (m, 1H), 2.53 (d, J=4.2 Hz, 1H), 2.03 (d, J=3.0 Hz, 7H), 1.25 (tdd, J=13.8, 8.9, 4.5 Hz, 2H), 0.76-0.60 (m, 1H), 0.39 (ddd, J=7.8, 5.5, 3.7 Hz, 2H), 0.08-−0.06 (m, 2H).

### Compound 660

### 3-(4-{[3-(aminomethyl)bicyclo[1.1.1]pentan-1-yl](2-cyclopropylethyl)amino}-1-oxo-3H-isoindol-2-yl)piperidine-2,6-dione

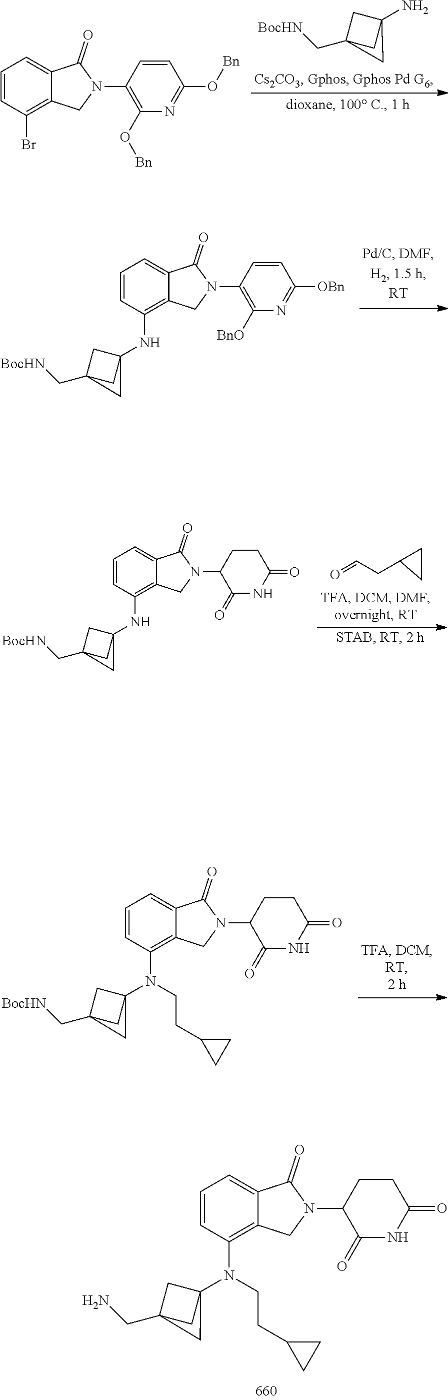

### Step-1: Synthesis of tert-butyl N-{[3-({2-[2,6-bis(benzyloxy)pyridin-3-yl]-1-oxo-3H-isoindol-4-yl}amino)bicyclo[1.1.1]pentan-1-yl]methyl}carbamate

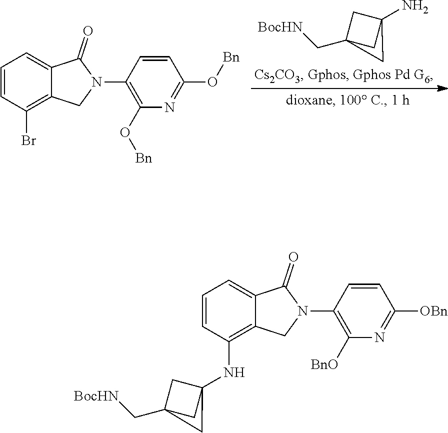 [SMILES: O=C1c2cccc(Br)c2CN1c1ccc(OCc2ccccc2)nc1OCc1ccccc1 ; CC(C)(C)OC(=O)NCC12CC(N)(C1)C2 ; CC(C)(C)OC(=O)NCC12CC(Nc3cccc4c3CN(c3ccc(OCc5ccccc5)nc3OCc3ccccc3)C4=O)(C1)C2]

To a stirred solution of 2-[2,6-bis(benzyloxy)pyridin-3-yl]-4-bromo-3H-isoindol-1-one (1.1 g, 2.19 mmol, 1 equiv) and tert-butyl N-({3-aminobicyclo[1.1.1]pentan-1-yl}methyl)carbamate (0.5 g, 2.19 mmol, 1 equiv) in dioxane (22 mL) were added Cs₂CO₃ (2.1 g, 6.58 mmol, 3 equiv), Gphos (240 mg, 0.439 mmol, 0.2 equiv) and GPhos Pd G6 (210 mg, 0.219 mmol, 0.1 equiv) at room temperature under nitrogen atmosphere. The resulting mixture was stirred for 1 h at 100° C. under nitrogen atmosphere. The resulting mixture was diluted with water (200 mL). The resulting mixture was extracted with EtOAc (3×200 mL). The combined organic layers were washed with brine (3×200 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 20% to 60% gradient in 10 min; detector, UV 254 nm to afford tert-butyl N-{[3-({2-[2,6-bis(benzyloxy)pyridin-3-yl]-1-oxo-3H-isoindol-4-yl}amino)bicyclo[1.1.1]pentan-1-yl]methyl}carbamate (0.7 g, 50%) as a light yellow solid.

LC/MS: mass calcd. for C₃₈H₄₀N₄O₅: 632.3 found: 633.3[mass+H]. RT: 1.22 min.

### Step-2: Synthesis of tert-butyl N-[(3-{[2-(2,6-dioxopiperidin-3-yl)-1-oxo-3H-isoindol-4-yl]amino}bicyclo[1.1.1]pentan-1-yl)methyl]carbamate

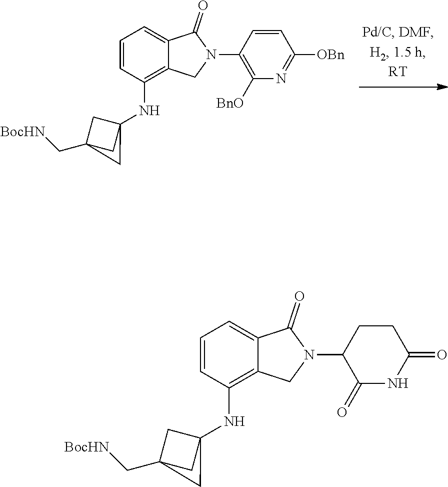 [SMILES: CC(C)(C)OC(=O)NCC12CC(Nc3cccc4c3CN(c3ccc(OCc5ccccc5)nc3OCc3ccccc3)C4=O)(C1)C2 ; CC(C)(C)OC(=O)NCC12CC(Nc3cccc4c3CN(C3CCC(=O)NC3=O)C4=O)(C1)C2]

To a stirred solution of tert-butyl N-{[3-({2-[2,6-bis(benzyloxy)pyridin-3-yl]-1-oxo-3H-isoindol-4-yl}amino)bicyclo[1.1.1]pentan-1-yl]methyl}carbamate (680 mg, 1.07 mmol, 1 equiv) in DMF (15 mL) was added Palladium 10% on Carbon (wetted with ca. 55% Water) (1.0 g, 0.94 mmol, 0.87 equiv) at room temperature under nitrogen atmosphere. The resulting mixture was stirred for 1.5 h at room temperature under hydrogen atmosphere. The resulting mixture was filtered, the filter cake was washed with CH₂Cl₂ (3×5 mL). The filtrate was concentrated under reduced pressure. The residue was purified by reversed-phase flash chromatography with the following conditions: column, C18 silica gel; mobile phase, MeCN in Water (0.1% FA), 30% to 50% gradient in 10 min; detector, UV 254 nm to afford tert-butyl N-[(3-{[2-(2,6-dioxopiperidin-3-yl)-1-oxo-3H-isoindol-4-yl]amino}bicyclo[1.1.1]pentan-1-yl)methyl]carbamate (450 mg, 92%) as a pink solid.

LC/MS: mass calcd. for C₂₄H₃₀N₄O₅: 453.4 found: 455.4[mass+H]. RT: 0.86 min.

### Step-3: Synthesis of tert-butyl N-({3-[(2-cyclopropylethyl)[2-(2,6-dioxopiperidin-3-yl)-1-oxo-3H-isoindol-4-yl]amino]bicyclo[1.1.1]pentan-1-yl}methyl)carbamate

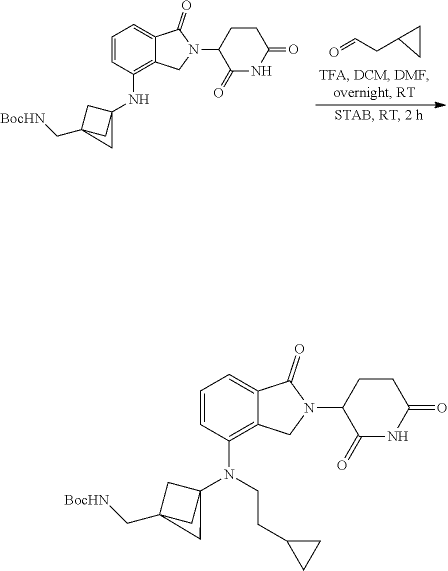

To a stirred solution of tert-butyl N-[(3-{[2-(2,6-dioxopiperidin-3-yl)-1-oxo-3H-isoindol-4-yl]amino}bicyclo[1.1.1]pentan-1-yl)methyl]carbamate (470 mg, 1.03 mmol, 1 equiv) and 2-cyclopropylacetaldehyde (1.7 g, 20.7 mmol, 20 equiv) in DCM (12 mL) and DMF (3 mL) was added TFA (354 mg, 3.1 mmol, 3 equiv) at room temperature under nitrogen atmosphere. The resulting mixture was stirred for overnight at room temperature under nitrogen atmosphere. To the above mixture was added STAB (1.1 g, 5.2 mmol, 5 equiv) in portions over 5 min at 0° C. The resulting mixture was stirred for additional 2 h at room temperature. The resulting mixture was diluted with water (100 mL). The resulting mixture was extracted with EtOAc (3×200 mL). The combined organic layers were washed with brine (3×200 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 50% to 70% gradient in 5 min; detector, UV 254 nm to afford tert-butyl N-({3-[(2-cyclopropylethyl)[2-(2,6-dioxopiperidin-3-yl)-1-oxo-3H-isoindol-4-yl]amino]bicyclo[1.1.1]pentan-1-yl}methyl)carbamate (450 mg, 83%) as an off-white solid.

LC/MS: mass calcd. for C₂₉H₃₈N₄O₅: 522.3 found: 523.3[mass+H]. RT: 0.99 min.

### Step-4: 3-(4-{[3-(aminomethyl)bicyclo[1.1.1]pentan-1-yl](2-cyclopropylethyl)amino}-1-oxo-3H-isoindol-2-yl)piperidine-2,6-dione

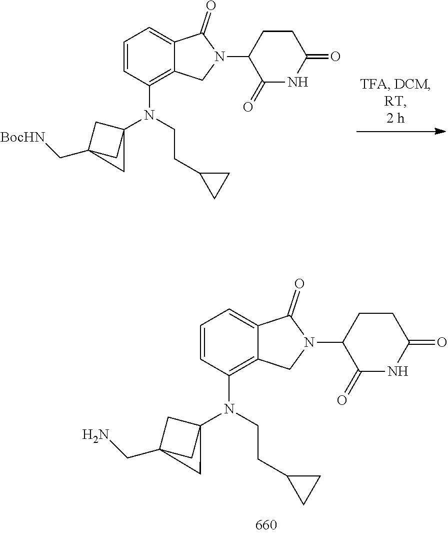 [SMILES: CC(C)(C)OC(=O)NCC12CC(N(CCC3CC3)c3cccc4c3CN(C3CCC(=O)NC3=O)C4=O)(C1)C2 ; NCC12CC(N(CCC3CC3)c3cccc4c3CN(C3CCC(=O)NC3=O)C4=O)(C1)C2]

To a stirred solution of tert-butyl N-({3-[(2-cyclopropylethyl)[2-(2,6-dioxopiperidin-3-yl)-1-oxo-3H-isoindol-4-yl]amino]bicyclo[1.1.1]pentan-1-yl}methyl)carbamate (440 mg, 0.84 mmol, 1 equiv) in DCM (6.6 mL) was added TFA (2.2 mL) at room temperature. The resulting mixture was stirred for 2 h at room temperature. The resulting mixture was concentrated under reduced pressure at 0° C. The residue was purified by reversed-phase flash chromatography with the following conditions: column, C18 silica gel; mobile phase, MeCN in Water (0.1% FA), 0% to 20% gradient in 10 min; detector, UV 254 nm to afford 3-(4-{[3-(aminomethyl)bicyclo[1.1.1]pentan-1-yl](2-cyclopropylethyl)amino}-1-oxo-3H-isoindol-2-yl)piperidine-2,6-dione (220 mg, 62%) as an off-white solid.

Compound 660: LCMS: 423.2 (M+H). Method: Mobile Phase A: Water/0.05% TFA. Mobile Phase B: Acetonitriler/0.05% TFA. Flow rate: 1.5 mL/min. COLUMN: ShimNex HE C18-AQ (30×3.0) mm, 3.0μ. Rt (min): 1.391; Area %—98.97. ¹H NMR (300 MHz, DMSO-d6): δ 8.43 (s, 1H), 7.59-7.50 (m, 2H), 7.45 (dd, J=6.5, 2.5 Hz, 1H), 5.18 (dd, J=13.1, 5.1 Hz, 1H), 4.47-4.24 (m, 2H), 3.22 (t, J=7.4 Hz, 2H), 3.08-2.88 (m, 3H), 2.75-2.60 (m, 1H), 2.51 (d, J=4.3 Hz, 1H), 2.14-1.97 (m, 1H), 1.83 (s, 6H), 1.23 (hept, J=6.7 Hz, 2H), 0.76-0.55 (m, 1H), 0.47-0.28 (m, 2H), 0.14-−0.08 (m, 2H).

### Compound 662

### 3-(4-[(2-cyclopropylethyl)[(1r,4r)-4-[(1,3-oxazol-2-ylmethyl)amino]cyclohexyl]amino]-1-oxo-3H-isoindol-2-ylpiperidine-2,6-dione

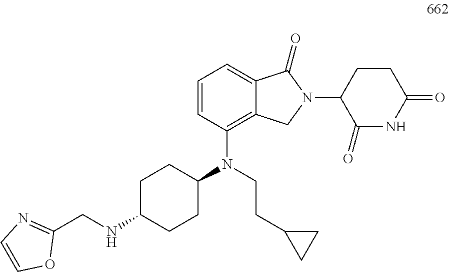 [SMILES: O=C1CCC(N2Cc3c(cccc3N(CCC3CC3)[C@H]3CC[C@H](NCc4ncco4)CC3)C2=O)C(=O)N1]

The compound was prepared by the same procedure described in the preparation of Compound 540 above using the appropriate aldehyde or ketone.

LC-MS: (ES, m/z): [M+1]=506.25. Method: Mobile phase A:Water/0.1% FA, Mobile phase B:Acetonitrile/0.1% FA. Flow rate: 1.5000 mL/min Column Name: HALO-C18 (30×2.0) mm, 3.0 μm. Rt (min): 4.96; Area %—98.641. ¹H NMR (400 MHz, DMSO-d₆): δ 10.96 (s, 1H), 8.19 (s, 1H), 7.42 (t, J=7.7 Hz, 1H), 7.28 (dd, J=16.7, 8.0 Hz, 3H), 5.11 (dd, J=13.2, 5.1 Hz, 1H), 4.37-4.19 (m, 4H), 3.19 (t, J=7.4 Hz, 2H), 3.09 (d, J=11.7 Hz, 1H), 2.97-2.84 (m, 2H), 2.59 (d, J=16.9 Hz, 1H), 2.00 (d, J=14.9 Hz, 3H), 1.80 (s, 2H), 1.53 (d, J=13.0 Hz, 2H), 1.30 (s, 2H), 1.13 (q, J=7.4 Hz, 2H), 0.63 (d, J=8.2 Hz, 1H), 0.32 (dd, J=8.0, 4.3 Hz, 2H), −0.08 (t, J=4.8 Hz, 2H).

### Compound 663

### 3-{4-[(2-cyclopropylethyl)[(1r,4r)-4-[(1,3-thiazol-2-ylmethyl)amino]cyclohexyl]amino]-1-oxo-3H-isoindol-2-yl}piperidine-2,6-dione

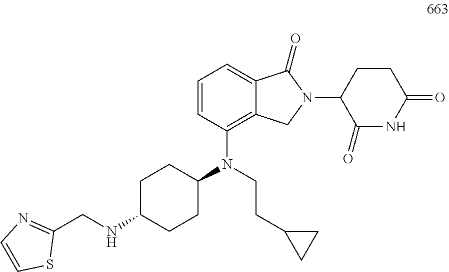 [SMILES: O=C1CCC(N2Cc3c(cccc3N(CCC3CC3)[C@H]3CC[C@H](NCc4nccs4)CC3)C2=O)C(=O)N1]

The compound was prepared by the same procedure described in the preparation of Compound 540 above using the appropriate aldehyde or ketone.

LC-MS: (ES, m/z): [M+1]=522.25. Method: Mobile Phase A: Water/0.1% FA, Mobile Phase B: Acetonitrile/0.1% FA. Flow rate:1.5000 mL/min. COLUMN: HALO-C18 (30×3.0) mm, 2μ, Rt (min):4.56; Area %—97.3397. ¹H NMR (400 MHz, DMSO-d6) δ 11.03 (s, 1H), 7.85 (d, J=3.3 Hz, 1H), 7.75 (d, J=3.3 Hz, 1H), 7.48 (t, J=7.6 Hz, 1H), 7.33 (dd, J=18.8, 7.6 Hz, 2H), 5.17 (dd, J=13.1, 5.1 Hz, 1H), 4.41 (d, J=17.2 Hz, 1H), 4.32 (d, J=10.4 Hz, 3H), 3.26 (t, J=7.3 Hz, 2H), 3.16 (td, J=7.9, 3.8 Hz, 1H), 3.01-2.94 (m, 1H), 2.76 (t, J=11.6 Hz, 1H), 2.65 (ddt, J=18.9, 12.4, 6.0 Hz, 2H), 2.56-2.50 (m, 1H), 2.08 (dq, J=11.5, 5.8, 5.1 Hz, 3H), 1.90-1.82 (m, 2H), 1.60 (dh, J=16.9, 4.3 Hz, 2H), 1.39-1.28 (m, 2H), 1.20 (q, J=7.2 Hz, 2H), 0.69 (pd, J=7.3, 3.6 Hz, 1H), 0.40 (dd, J=8.8, 4.4 Hz, 2H).

### Compound 664

### 3-{4-[(2-cyclopropylethyl)[(1r,4r)-4-({[5-(trifluoromethyl)pyridin-2-yl]methyl}amino)cyclohexyl]amino]-1-oxo-3H-isoindol-2-yl}piperidine-2,6-dione formic acid

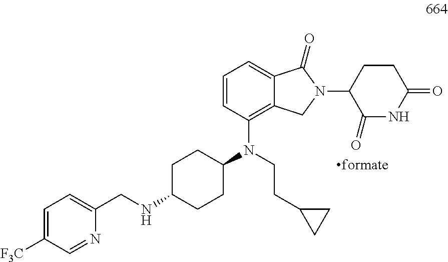 [SMILES: O=C1CCC(N2Cc3c(cccc3N(CCC3CC3)[C@H]3CC[C@H](NCc4ccc(C(F)(F)F)cn4)CC3)C2=O)C(=O)N1]

The compound was prepared by the same procedure described in the preparation of Compound 540 above using the appropriate aldehyde or ketone.

LCMS: 584.25 (M+H). Method: Mobile Phase A: Water/0.1% FA. Mobile Phase B: Acetonitriler/0.1% FA. Flow rate:1.5 mL/min. COLUMN: HALO-C18 (30×3.0) mm, 2.0μ. Rt (min): 0.977; Area %—99.887. ¹H NMR (400 MHz, DMSO-d₆): δ 11.04 (s, 1H), 8.97 (s, 1H), 8.26 (d, J=9.0 Hz, 2H), 7.79 (d, J=8.3 Hz, 1H), 7.47 (t, J=7.7 Hz, 1H), 7.37-7.29 (m, 2H), 5.17 (dd, J=13.5, 5.0 Hz, 1H), 4.44-4.32 (m, 2H), 4.09 (s, 3H), 3.27 (s, 2H), 3.16 (s, 1H), 3.01-2.93 (m, 1H), 2.66 (d, J=18.0 Hz, 2H), 2.05 (s, 3H), 1.82 (s, 2H), 1.58 (s, 2H), 1.23 (t, J=15.8 Hz, 5H), 0.70 (s, 1H), 0.39 (d, J=7.9 Hz, 2H).

### Compound 665

### 3-{4-[(2-cyclopropylethyl)[(1r,4r)-4-[(pyridin-2-ylmethyl)amino]cyclohexyl]amino]-1-oxo-3H-isoindol-2-yl}piperidine-2,6-dione; formic acid

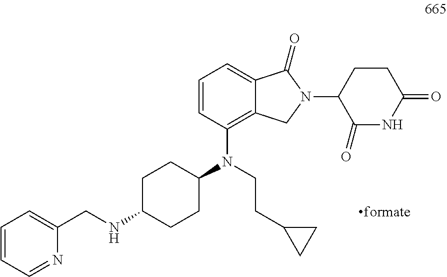 [SMILES: O=C1CCC(N2Cc3c(cccc3N(CCC3CC3)[C@H]3CC[C@H](NCc4ccccn4)CC3)C2=O)C(=O)N1]

The compound was prepared by the same procedure described in the preparation of Compound 540 above using the appropriate aldehyde or ketone.

LC-MS: (ES, m/z): [M+1]=516.35. ¹H NMR: ¹H NMR (400 MHz, DMSO-d6) δ 10.96 (s, 1H), 8.49 (d, J=4.8 Hz, 1H), 8.25 (s, 1H), 7.75 (td, J=7.5, 1.8 Hz, 1H), 7.52-7.38 (m, 2H), 7.32-7.20 (m, 3H), 5.09 (dd, J=13.2, 5.1 Hz, 1H), 4.39-4.19 (m, 2H), 3.87 (s, 2H), 3.19 (t, J=7.3 Hz, 2H), 3.13-3.05 (m, 1H), 2.90 (ddd, J=17.7, 13.5, 5.4 Hz, 1H), 2.66-2.55 (m, 1H), 2.45 (d, J=4.3 Hz, 1H), 2.07-1.91 (m, 3H), 1.75 (d, J=11.4 Hz, 2H), 1.50 (d, J=13.3 Hz, 2H), 1.28-1.07 (m, 4H), 0.62 (tq, J=12.1, 6.7, 6.1 Hz, 1H), 0.38-0.25 (m, 2H), 0.02-−0.12 (m, 2H).

### Compound 666

### 3-{4-[(2-cyclopropylethyl)[(1r,4r)-4-[(oxolan-2-ylmethyl)amino]cyclohexyl]amino]-1-oxo-3H-isoindol-2-yl}piperidine-2,6-dione

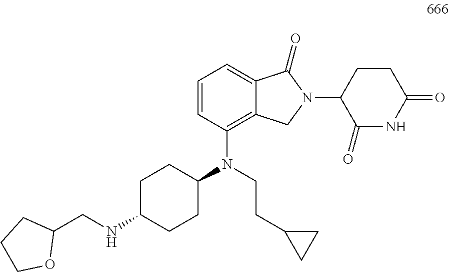 [SMILES: O=C1CCC(N2Cc3c(cccc3N(CCC3CC3)[C@H]3CC[C@H](NCC4CCCO4)CC3)C2=O)C(=O)N1]

The compound was prepared by the same procedure described in the preparation of Compound 540 above using the appropriate aldehyde or ketone.

LCMS: 509.30 (M+H). Method: Mobile Phase A: Water/0.05% TFA. Mobile Phase B: Acetonitrile/0.05% TFA. Flow rate: 1.5 mL/min. COLUMN: EC-C18 (30×3.0) mm, 1.9μ. Rt (min): 1.052; Area %—96.12. ¹H NMR (400 MHz, DMSO-d6): δ 11.02 (s, 1H), 7.46 (t, J=7.7 Hz, 1H), 7.30 (dd, J=19.6, 7.7 Hz, 2H), 5.15 (dd, J=13.2, 5.1 Hz, 1H), 4.46-4.25 (m, 2H), 3.87 (p, J=6.4 Hz, 1H), 3.77 (q, J=7.0 Hz, 1H), 3.64 (q, J=7.2 Hz, 1H), 3.29-3.22 (m, 2H), 3.13 (d, J=12.3 Hz, 1H), 2.96 (ddd, J=17.7, 13.6, 5.4 Hz, 1H), 2.76-2.59 (m, 3H), 2.40 (s, 2H), 2.13-2.00 (m, 1H), 1.94 (q, J=13.1, 9.4 Hz, 3H), 1.87-1.75 (m, 4H), 1.65-1.49 (m, 3H), 1.30 (s, 1H), 1.24-1.03 (m, 4H), 0.69 (q, J=6.5 Hz, 1H), 0.46-0.35 (m, 2H), 0.06 (s, 2H).

### Compound 668

### 3-{4-[(2-cyclopropylethyl)[(1r,4r)-4-(dimethylamino)cyclohexyl]amino]-1-oxo-3H-isoindol-2-yl}piperidine-2,6-dione

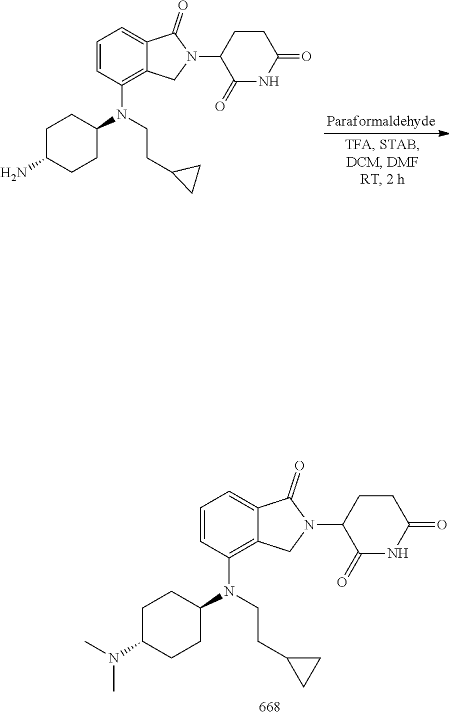 [SMILES: N[C@H]1CC[C@H](N(CCC2CC2)c2cccc3c2CN(C2CCC(=O)NC2=O)C3=O)CC1 ; CN(C)[C@H]1CC[C@H](N(CCC2CC2)c2cccc3c2CN(C2CCC(=O)NC2=O)C3=O)CC1]

A solution of 3-{4-[(2-cyclopropylethyl)[(1r,4r)-4-aminocyclohexyl]amino]-1-oxo-3H-isoindol-2-yl}piperidine-2,6-dione (150 mg, 0.35 mmol, 1 equiv), Paraformaldehyde (47 mg, 1.06 mmol, 3 equiv) in DCM (4 mL) and DMF (1 mL) was treated with Trifluoroacetic acid (173 mg, 1.76 mmol, 5 equiv) for overnight at room temperature under nitrogen atmosphere followed by the addition of STAB (374 mg, 1.76 mmol, 5 equiv) at 0° C. The resulting mixture was stirred for additional 2 h at room temperature. The reaction was diluted with water and extracted with EtOAc (3×20 mL). The combined organic layers were washed with brine (2×3 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 30% to 70% gradient in 10 min; detector, UV 254 nm. This resulted in 3-{4-[(2-cyclopropylethyl)[(1r,4r)-4-(dimethylamino)cyclohexyl]amino]-1-oxo-3H-isoindol-2-yl}piperidine-2,6-dione (24 mg, 15%) as an off-white solid.

LCMS: 453.30 (M+H). Method: Mobile Phase A: Water/0.1% FA. Mobile Phase B: Acetonitriler/0.1% FA. Flow rate:1.5 mL/min. COLUMN: HALO-C18 (30×3.0) mm, 2.0μ. Rt (min):0.873; Area %—99.689. ¹H NMR (400 MHz, DMSO-d6):δ 11.02 (s, 1H), 8.30 (s, 1H), 7.47 (t, J=7.7 Hz, 1H), 7.33 (dd, J=15.8, 7.6 Hz, 2H), 5.15 (dd, J=13.1, 5.1 Hz, 1H), 4.41 (d, J=17.2 Hz, 1H), 4.31 (d, J=17.1 Hz, 1H), 3.25 (p, J=6.6 Hz, 2H), 3.14 (s, 1H), 2.96 (ddd, J=18.4, 13.9, 5.3 Hz, 1H), 2.70-2.61 (m, 1H), 2.50 (d, J=11.7 Hz, 1H), 2.37 (s, 6H), 2.12-2.04 (m, 1H), 1.97-1.85 (m, 4H), 1.59 (h, J=10.9 Hz, 2H), 1.36 (td, J=20.7, 18.9, 9.1 Hz, 2H), 1.20 (q, J=7.0 Hz, 2H), 0.74-0.66 (m, 1H), 0.44-0.35 (m, 2H).

### Compounds 669 and 669A

### 3-(4-[(2-cyclopropylethyl)[(1r,4r)-4-(pyrrolidin-1-yl)cyclohexyl]amino]-1-oxo-3H-isoindol-2-ylpiperidine-2,6-dione and 3-(4-[(2-cyclopropylethyl)[(1s,4s)-4-(pyrrolidin-1-yl)cyclohexyl]amino]-1-oxo-3H-isoindol-2-ylpiperidine-2,6-dione

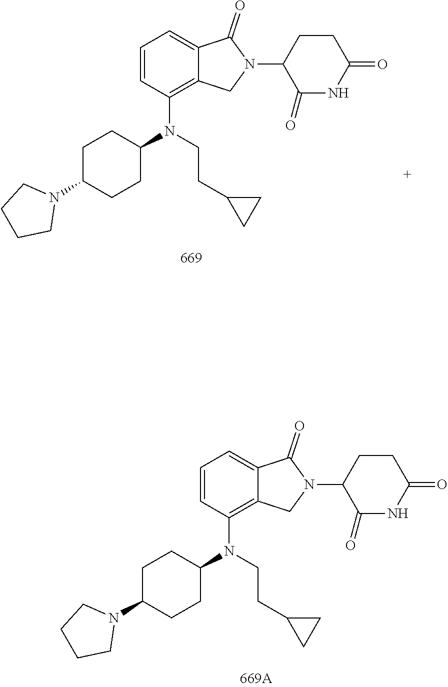 [SMILES: O=C1CCC(N2Cc3c(cccc3N(CCC3CC3)[C@H]3CC[C@H](N4CCCC4)CC3)C2=O)C(=O)N1 ; O=C1CCC(N2Cc3c(cccc3N(CCC3CC3)[C@H]3CC[C@@H](N4CCCC4)CC3)C2=O)C(=O)N1]

The compounds were prepared by the general procedure described in the preparation of Compounds 515 and 515A using the appropriate amine.

Compound 669: LC-MS: (ES, m/z): [M+1]=479.20 (M+H). ¹H NMR (400 MHz, DMSO-d₆): δ 10.97 (s, 1H), 7.42 (t, J=7.6 Hz, 1H), 7.30-7.22 (m, 2H), 5.11 (dd, J=13.2, 5.1 Hz, 1H), 4.35 (d, J=17.1 Hz, 1H), 4.24 (d, J=17.1 Hz, 1H), 3.20 (t, J=5.5 Hz, 2H), 3.09 (d, J=11.0 Hz, 1H), 2.92 (td, J=13.2, 12.2, 6.7 Hz, 5H), 2.61 (d, J=3.6 Hz, 1H), 2.03 (s, 3H), 1.79 (d, J=9.1 Hz, 6H), 1.52 (d, J=11.0 Hz, 2H), 1.37 (s, 2H), 1.13 (t, J=7.1 Hz, 2H), 0.64 (t, J=7.4 Hz, 1H), 0.34 (dd, J=9.0, 4.4 Hz, 2H), −0.07 (d, J=4.8 Hz, 2H).

Compound 669A: LC-MS: (ES, m/z): [M+1]=479.35 (M+H). ¹H NMR (400 MHz, DMSO-d₆): δ 10.98 (s, 1H), 7.43 (t, J=7.6 Hz, 1H), 7.32 (dd, J=15.7, 7.6 Hz, 2H), 5.10 (dd, J=13.2, 5.2 Hz, 1H), 4.39 (d, J=17.2 Hz, 1H), 4.28 (d, J=17.2 Hz, 1H), 3.19 (q, J=6.4 Hz, 2H), 2.91 (ddd, J=18.2, 13.6, 5.4 Hz, 4H), 2.62 (d, J=3.8 Hz, 1H), 2.58 (s, 1H), 2.07-1.98 (m, 1H), 1.85 (q, J=11.3, 9.2 Hz, 8H), 1.56 (d, J=44.9 Hz, 4H), 1.17 (q, J=7.6 Hz, 2H), 0.61 (d, J=7.9 Hz, 1H), 0.38-0.29 (m, 2H), −0.07 (q, J=4.8 Hz, 2H).

### Compounds 670 and 670A

### 3-(4-[(2-cyclopropylethyl)[(1r,4r)-4-(3,3-difluoropyrrolidin-1-yl)cyclohexyl]amino]-1-oxo-3H-isoindol-2-ylpiperidine-2,6-dione and 3-(4-[(2-cyclopropylethyl)[(1s,4s)-4-(3,3-difluoropyrrolidin-1-yl)cyclohexyl]amino]-1-oxo-3H-isoindol-2-ylpiperidine-2,6-dione

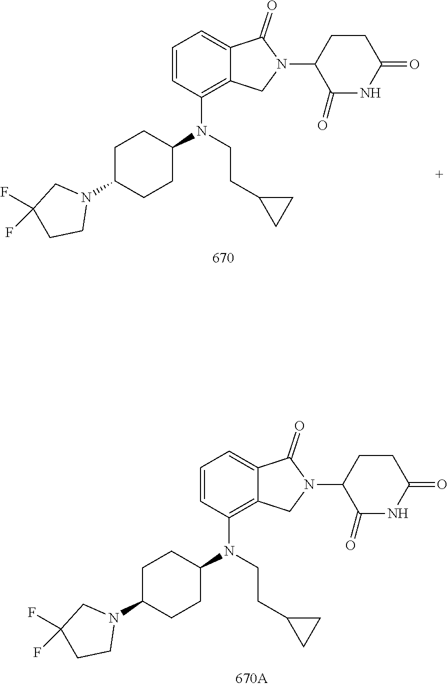 [SMILES: O=C1CCC(N2Cc3c(cccc3N(CCC3CC3)[C@H]3CC[C@H](N4CCC(F)(F)C4)CC3)C2=O)C(=O)N1 ; O=C1CCC(N2Cc3c(cccc3N(CCC3CC3)[C@H]3CC[C@@H](N4CCC(F)(F)C4)CC3)C2=O)C(=O)N1]

The compounds were prepared by the general procedure described in the preparation of Compounds 515 and 515A using the appropriate amine.

Compound 670: LC-MS: (ES, m/z): [M+1]=515.25 (M+H). ¹H NMR (400 MHz, DMSO-d₆): δ 10.96 (s, 1H), 7.40 (t, J=7.6 Hz, 1H), 7.24 (dd, J=18.7, 7.7 Hz, 2H), 5.09 (dd, J=13.1, 5.1 Hz, 1H), 4.40-4.23 (m, 2H), 3.19 (t, J=5.6 Hz, 2H), 3.09 (d, J=12.0 Hz, 1H), 2.89 (t, J=13.6 Hz, 3H), 2.70 (t, J=7.0 Hz, 2H), 2.59 (d, J=17.6 Hz, 1H), 2.20 (dt, J=14.6, 7.1 Hz, 2H), 2.10-1.99 (m, 2H), 1.91 (d, J=12.0 Hz, 2H), 1.75 (s, 2H), 1.56-1.46 (m, 2H), 1.26-1.12 (m, 5H), 0.63 (q, J=6.7 Hz, 1H), 0.33 (d, J=7.8 Hz, 2H), −0.07 (d, J=4.9 Hz, 2H).

Compound 670A: LC-MS: (ES, m/z): [M+1]=515.30 (M+H). ¹H NMR (400 MHz, DMSO-d₆): δ 10.95 (s, 1H), 7.40 (t, J=7.7 Hz, 1H), 7.26 (dd, J=13.2, 7.7 Hz, 2H), 5.11-5.06 (m, 1H), 4.31 (d, J=21.0 Hz, 2H), 3.19 (d, J=5.2 Hz, 3H), 2.86 (d, J=13.5 Hz, 3H), 2.70-2.57 (m, 3H), 2.29 (d, J=8.0 Hz, 2H), 2.02 (s, 1H), 1.79 (d, J=11.8 Hz, 4H), 1.44 (s, 4H), 1.24 (s, 1H), 1.16 (t, J=7.2 Hz, 2H), 0.62 (s, 1H), 0.33 (d, J=7.9 Hz, 2H), −0.07 (d, J=4.9 Hz, 2H).

### Compounds 671 and 671A

### 3-{4-[(2-cyclopropylethyl)[(1r,4r)-4-(3-fluoropyrrolidin-1-yl)cyclohexyl]amino]-1-oxo-3H-isoindol-2-yl}piperidine-2,6-dione and 3-{4-[(2-cyclopropylethyl)[(1s,4s)-4-(3-fluoropyrrolidin-1-yl)cyclohexyl]amino]-1-oxo-3H-isoindol-2-yl}piperidine-2,6-dione

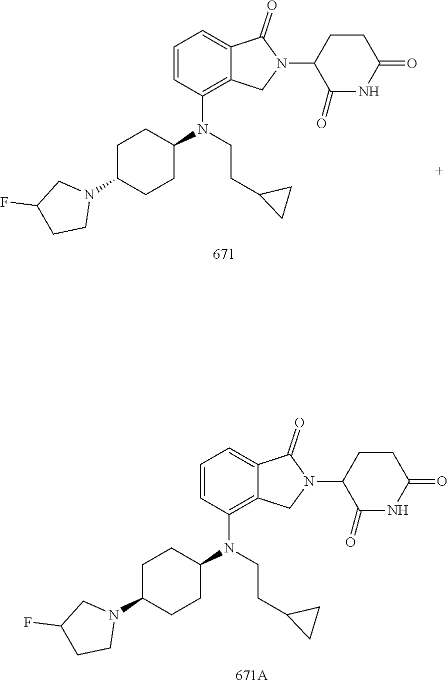 [SMILES: O=C1CCC(N2Cc3c(cccc3N(CCC3CC3)[C@H]3CC[C@H](N4CCC(F)C4)CC3)C2=O)C(=O)N1 ; O=C1CCC(N2Cc3c(cccc3N(CCC3CC3)[C@H]3CC[C@@H](N4CCC(F)C4)CC3)C2=O)C(=O)N1]

The compounds were prepared by the general procedure described in the preparation of Compounds 515 and 515A using the appropriate amine.

Compound 671: LCMS: 497.40 (M+H). ¹H NMR (400 MHz, DMSO-d₆): δ 10.96 (s, 1H), 7.41 (d, J=7.7 Hz, 1H), 7.24 (dd, J=18.7, 7.6 Hz, 2H), 5.27-5.07 (m, 2H), 4.35-4.22 (m, 2H), 3.22-3.18 (m, 2H), 3.10 (s, 1H), 2.96-2.74 (m, 4H), 2.61 (t, J=12.9 Hz, 2H), 2.35-2.29 (m, 1H), 2.06-1.91 (m, 5H), 1.74 (d, J=11.0 Hz, 3H), 1.52 (s, 2H), 1.24 (s, 2H), 1.15 (d, J=7.1 Hz, 2H), 0.63 (d, J=5.7 Hz, 1H), 0.37-0.28 (m, 2H), −0.07 (dt, J=5.7, 2.9 Hz, 2H).

Compound 671A: LCMS: 497.40 (M+H). ¹H NMR (400 MHz, DMSO-d₆): δ 11.03 (s, 1H), 7.47 (t, J=7.6 Hz, 1H), 7.33 (dd, J=12.9, 7.6 Hz, 2H), 5.36-5.11 (m, 2H), 4.50-4.31 (m, 2H), 3.27 (p, J=7.1, 5.2 Hz, 3H), 3.02-2.82 (m, 4H), 2.70-2.62 (m, 2H), 2.39 (q, J=7.6 Hz, 1H), 2.20 (s, 2H), 2.16-2.02 (m, 2H), 1.89 (d, J=11.6 Hz, 4H), 1.51 (s, 4H), 1.29 (d, J=17.5 Hz, 1H), 1.24 (d, J=7.4 Hz, 2H), 0.68 (q, J=6.7, 6.3 Hz, 1H), 0.41 (dd, J=8.4, 4.6 Hz, 2H).

### Compounds 672 and 672A

### 3-{4-[(2-cyclopropylethyl)[(1r,4r)-4-[3-(trifluoromethyl)pyrrolidin-1-yl]cyclohexyl]amino]-1-oxo-3H-isoindol-2-yl}piperidine-2,6-dione; trifluoroacetic acid and 3-{4-[(2-cyclopropylethyl)[(1s,4s)-4-[3-(trifluoromethyl)pyrrolidin-1-yl]cyclohexyl]amino]-1-oxo-3H-isoindol-2-yl}piperidine-2,6-dione; trifluoroacetic acid

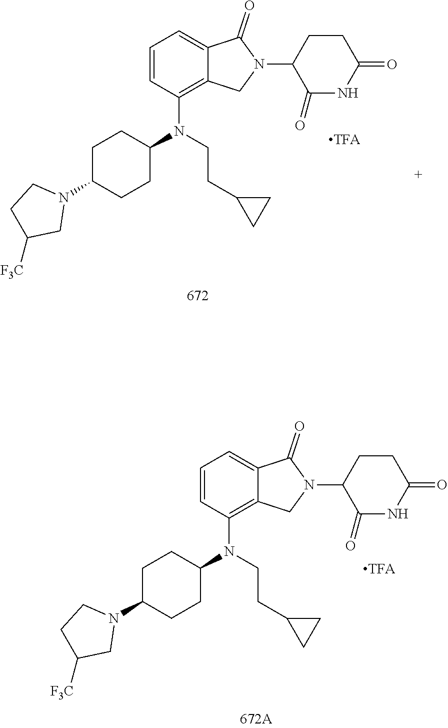 [SMILES: O=C1CCC(N2Cc3c(cccc3N(CCC3CC3)[C@H]3CC[C@H](N4CCC(C(F)(F)F)C4)CC3)C2=O)C(=O)N1 ; O=C1CCC(N2Cc3c(cccc3N(CCC3CC3)[C@H]3CC[C@@H](N4CCC(C(F)(F)F)C4)CC3)C2=O)C(=O)N1]

The compounds were prepared by the general procedure described in the preparation of Compounds 515 and 515A using the appropriate amine.

Compound 672: LCMS: 547.25 (M+H). Method: Mobile Phase A: Water/5 mM NH₄HCO₃. Mobile Phase B: Acetonitrile. Flow rate: 1.5 mL/min. COLUMN: kinetex EVO C18 (50×3.0) mm, 2.6μ. Rt (min): 1.663; Area %—97.96. ¹H NMR (400 MHz, DMSO-d6): δ 11.03 (s, 1H), 10.45 (s, 1H), 7.49 (t, J=7.6 Hz, 1H), 7.37 (dd, J=13.0, 7.7 Hz, 2H), 5.17 (dd, J=13.2, 5.1 Hz, 1H), 4.41 (d, J=17.2 Hz, 1H), 4.30 (d, J=17.2 Hz, 1H), 3.59 (s, 1H), 3.33-3.26 (m, 2H), 3.21 (dt, J=28.7, 8.6 Hz, 4H), 2.98 (ddd, J=17.9, 13.7, 5.4 Hz, 1H), 2.66 (dt, J=17.2, 3.4 Hz, 1H), 2.61-2.47 (m, 5H), 2.20 (t, J=11.0 Hz, 3H), 2.07 (tq, J=6.9, 4.7, 3.3 Hz, 1H), 1.93 (t, J=11.4 Hz, 2H), 1.61 (dd, J=13.1, 9.2 Hz, 2H), 1.55 (s, 1H), 1.20 (q, J=7.1 Hz, 2H), 0.69 (ddt, J=10.0, 7.3, 3.6 Hz, 1H), 0.44-0.33 (m, 2H), 0.06 (s, 2H). ¹⁹F NMR (377 MHz, DMSO-d6): δ −66.63 (s,1), −74.31 (s,1).

Compound 672A: LCMS: 547.25 (M+H). Method: Mobile Phase A: Water/5 mM NH4HCO3. Mobile Phase B: Acetonitrile. Flow rate: 1.5 mL/min. COLUMN: kinetex EVO C18 (50×3.0) mm, 2.6μ. Rt (min): 1.845; Area %—98.62. ¹H NMR (400 MHz, DMSO-d6): δ 11.07 (s, 1H), 10.56-10.07 (m, 1H), 7.57-7.39 (m, 3H), 5.19 (dd, J=13.2, 5.1 Hz, 1H), 4.49 (d, J=17.6 Hz, 1H), 4.38 (d, J=17.3 Hz, 1H), 4.10 (s, 1H), 3.86 (s, 1H), 3.45 (s, 1H), 3.26 (tq, J=16.5, 9.5, 8.0 Hz, 4H), 3.00 (ddd, J=18.0, 13.5, 5.4 Hz, 1H), 2.73-2.64 (m, 1H), 2.58-2.47 (m, 3H), 2.38-2.22 (m, 1H), 2.11 (dt, J=11.6, 5.5 Hz, 2H), 1.95 (s, 4H), 1.82 (d, J=11.9 Hz, 2H), 1.62 (d, J=12.1 Hz, 2H), 1.28 (dd, J=19.4, 12.0 Hz, 3H), 0.66 (q, J=6.9 Hz, 1H), 0.41 (dd, J=8.5, 4.4 Hz, 2H), 0.08 (s, 2H). ¹⁹F NMR (377 MHz, DMSO-d₆): δ −69.62 (s,1), −74.11 (s,1).

### Compounds 673 and 673A

### 3-(4-[(2-cyclopropylethyl)[(1r,4r)-4-[3-(trifluoromethyl)azetidin-1-yl]cyclohexyl]amino]-1-oxo-3H-isoindol-2-ylpiperidine-2,6-dione; trifluoroacetic acid and 3-(4-[(2-cyclopropylethyl)[(1s,4s)-4-[3-(trifluoromethyl)azetidin-1-yl]cyclohexyl]amino]-1-oxo-3H-isoindol-2-ylpiperidine-2,6-dione; trifluoroacetic acid

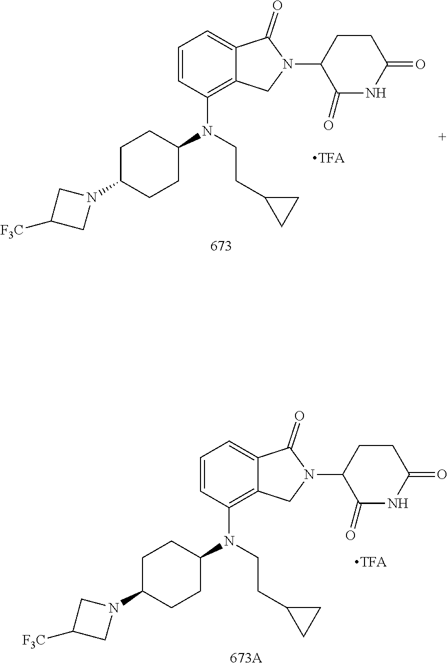 [SMILES: O=C1CCC(N2Cc3c(cccc3N(CCC3CC3)[C@H]3CC[C@H](N4CC(C(F)(F)F)C4)CC3)C2=O)C(=O)N1 ; O=C1CCC(N2Cc3c(cccc3N(CCC3CC3)[C@H]3CC[C@@H](N4CC(C(F)(F)F)C4)CC3)C2=O)C(=O)N1]

The compounds were prepared by the general procedure described in the preparation of Compounds 515 and 515A using the appropriate amine.

Compound 673: LC-MS: (ES, m/z): [M+1]=533.20. Method: Mobile phase A:Water/5 mM NH₄HCO₃, Mobile phase B:Acetonitrile. Flow rate: 1.2000 mL/min Column Name: EVO C18 (50×3.0) mm, 2.6 μm. Rt (min): 1.865; Area %—96.278. ¹H NMR (400 MHz, DMSO-d₆): δ 10.97 (s, 1H), 10.53 (d, J=72.0 Hz, 1H), 7.43 (t, J=7.6 Hz, 1H), 7.30 (dd, J=12.5, 7.6 Hz, 2H), 5.14-5.09 (m, 1H), 4.36 (s, 1H), 4.32-4.20 (m, 5H), 3.95-3.86 (m, 1H), 3.19 (q, J=6.2 Hz, 2H), 3.11 (d, J=12.9 Hz, 2H), 2.93 (td, J=13.0, 12.5, 6.8 Hz, 1H), 2.59 (dd, J=17.1, 3.6 Hz, 1H), 2.48-2.41 (m, 1H), 2.04-1.94 (m, 3H), 1.86 (s, 2H), 1.52 (s, 2H), 1.24-1.11 (m, 4H), 0.68-0.59 (m, 1H), 0.38-0.29 (m, 2H), −0.07 (dd, J=5.5, 4.2 Hz, 2H). ¹⁹F NMR (376 MHz, DMSO-d₆) δ −70.56 (s,1), −74.29 (s,2).

Compound 673A: LC-MS: (ES, m/z): [M+1]=533.30 (M+H). Method: Mobile phase A:Water/5 mM NH₄HCO₃ Mobile phase B:Acetonitrile. Flow rate: 1.2000 mL/min Column Name: EVO C18 (50×3.0) mm, 2.6 μm. Rt (min): 2.020; Area %—97.465. ¹H NMR (400 MHz, DMSO-d₆): δ 10.98 (d, J=2.9 Hz, 1H), 7.48-7.25 (m, 3H), 5.10 (dd, J=13.2, 5.1 Hz, 1H), 4.44-4.36 (m, 3H), 4.26 (d, J=17.5 Hz, 2H), 3.92 (q, J=8.6 Hz, 1H), 3.19 (dq, J=13.7, 6.7 Hz, 4H), 2.92 (ddd, J=17.9, 13.6, 5.4 Hz, 1H), 2.64-2.55 (m, 1H), 2.46 (d, J=4.5 Hz, 1H), 2.06-1.97 (m, 1H), 1.81-1.60 (m, 6H), 1.51 (s, 2H), 1.15 (q, J=7.2 Hz, 2H), 0.59 (d, J=10.2 Hz, 1H), 0.32 (dt, J=8.5, 2.5 Hz, 2H), −0.08 (q, J=4.7 Hz, 2H). ¹⁹F NMR (376 MHz, DMSO-d₆) δ −70.68 (s,1), −74.12 (s,1).

### Compounds 674 and 674A

### 3-{4-[(2-cyclopropylethyl)[(1r,4r)-4-(morpholin-4-yl)cyclohexyl]amino]-1-oxo-3H-isoindol-2-yl}piperidine-2,6-dione and 3-{4-[(2-cyclopropylethyl)[(1s,4s)-4-(morpholin-4-yl)cyclohexyl]amino]-1-oxo-3H-isoindol-2-yl}piperidine-2,6-dione

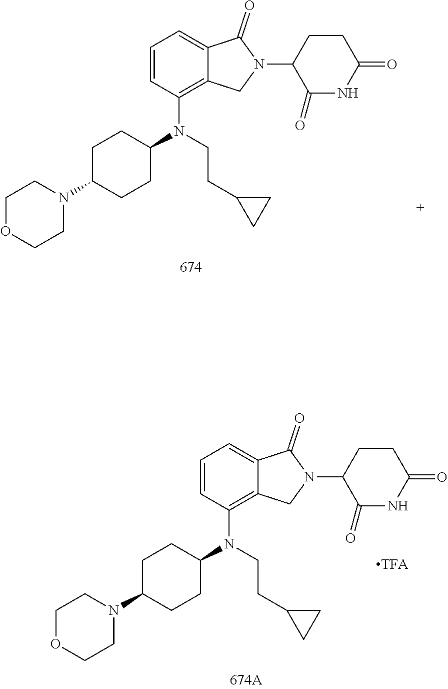 [SMILES: O=C1CCC(N2Cc3c(cccc3N(CCC3CC3)[C@H]3CC[C@H](N4CCOCC4)CC3)C2=O)C(=O)N1 ; O=C1CCC(N2Cc3c(cccc3N(CCC3CC3)[C@H]3CC[C@@H](N4CCOCC4)CC3)C2=O)C(=O)N1]

The compounds were prepared by the general procedure described in the preparation of Compounds 515 and 515A using the appropriate amine.

Compound 674: LCMS: 495.30 (M+H). Method: Mobile Phase A: Water/0.05% TFA. Mobile Phase B: Acetonitrile/0.05% TFA. Flow rate:1.5 mL/min. COLUMN: EC-C18 (30×3.0) mm, 1.9μ. Rt (min): 1.092; Area %—95.222. ¹H NMR (400 MHz, DMSO-d₆):δ 11.03 (s, 1H), 7.46 (t, J=7.7 Hz, 1H), 7.31 (dd, J=17.7, 7.7 Hz, 2H), 5.15 (dd, J=13.2, 5.2 Hz, 1H), 4.45-4.23 (m, 2H), 3.60 (t, J=4.5 Hz, 4H), 3.26 (td, J=6.9, 3.3 Hz, 2H), 3.12 (s, 1H), 2.96 (ddd, J=18.0, 13.5, 5.3 Hz, 1H), 2.70-2.61 (m, 1H), 2.50 (t, J=4.6 Hz, 4H), 2.23 (t, J=11.6 Hz, 1H), 2.08 (dd, J=12.3, 5.8 Hz, 1H), 1.88 (t, J=15.5 Hz, 4H), 1.58 (t, J=11.0 Hz, 2H), 1.29 (q, J=11.7, 10.4 Hz, 2H), 1.20 (q, J=7.0 Hz, 2H), 0.69 (h, J=6.5 Hz, 1H), 0.44-0.33 (m, 2H).

Compound 674A: LCMS: 495.25 (M+H). Method: Mobile Phase A: Water/0.05% TFA. Mobile Phase B: Acetonitrile/0.05% TFA. Flow rate:1.5 mL/min. COLUMN: EC-C18 (30×3.0) mm, 1.9μ. Rt (min): 1.290; Area %—98.268. ¹H NMR (400 MHz, DMSO-d₆): δ 11.08 (s, 1H), 7.53 (d, J=2.1 Hz, 3H), 5.22 (d, J=8.1 Hz, 1H), 4.47 (s, 1H), 4.40 (s, 1H), 4.07 (s, 2H), 3.80 (s, 2H), 3.51 (s, 2H), 3.34-3.32 (m, 1H), 3.18 (d, J=8.4 Hz, 4H), 3.01 (t, J=4.6 Hz, 1H), 2.71 (d, J=4.0 Hz, 1H), 2.51 (dd, J=13.2, 4.5 Hz, 1H), 2.13-2.10 (m, 1H), 2.02-1.98 (m, 2H), 1.89 (d, J=10.8 Hz, 1H), 1.78 (d, J=5.1 Hz, 3H), 1.59-1.55 (m, 2H), 1.30-1.26 (m, 2H), 0.64 (s, 1H), 0.40 (d, J=3.9 Hz, 2H). ¹⁹F NMR (400 MHz, DMSO-d₆): δ 74.043 (s, 1).

### Compounds 675 and 675

### 3-{4-[(2-cyclopropylethyl)[(1r,4r)-4-{[(1S,2S)-2-(trifluoromethyl)cyclobutyl]amino}cyclohexyl]amino]-1-oxo-3H-isoindol-2-yl}piperidine-2,6-dione; bis(trifluoroacetic acid) and 3-{4-[(2-cyclopropylethyl)[(1s,4s)-4-{[(1S,2S)-2-(trifluoromethyl)cyclobutyl]amino}cyclohexyl]amino]-1-oxo-3H-isoindol-2-yl}piperidine-2,6-dione; bis(trifluoroacetic acid)

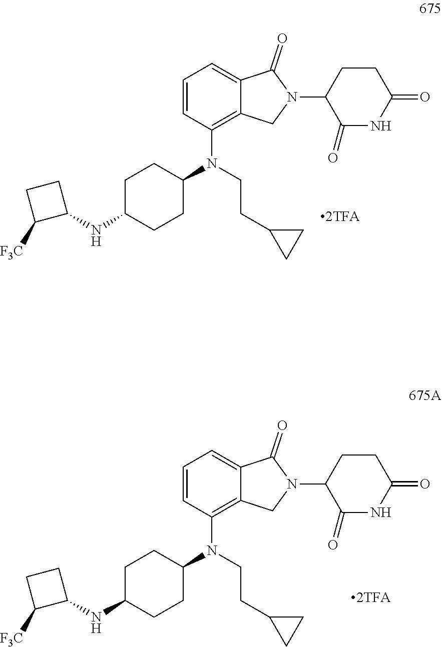 [SMILES: O=C1CCC(N2Cc3c(cccc3N(CCC3CC3)[C@H]3CC[C@H](N[C@H]4CC[C@@H]4C(F)(F)F)CC3)C2=O)C(=O)N1 ; O=C1CCC(N2Cc3c(cccc3N(CCC3CC3)[C@H]3CC[C@@H](N[C@H]4CC[C@@H]4C(F)(F)F)CC3)C2=O)C(=O)N1]

The compounds were prepared by the general procedure described in the preparation of Compounds 515 and 515A using the appropriate amine.

Compound 675: LCMS: 547.30 (M+H). Method: Mobile Phase A: Water/0.05% TFA. Mobile Phase B: Acetonitrile/0.05% TFA. Flow rate: 1.5 mL/min. COLUMN: EC-C18 (50×3.0) mm, 1.9μ. Rt (min): 1.290; Area %—99.561. ¹H NMR (400 MHz, DMSO-d₆): δ 11.02 (s, 1H), 9.15 (d, J=21.0 Hz, 2H), 7.49 (t, J=7.6 Hz, 1H), 7.36 (dd, J=15.6, 7.6 Hz, 2H), 5.18 (dd, J=13.2, 5.1 Hz, 1H), 4.40 (d, J=17.2 Hz, 2H), 4.29 (d, J=17.2 Hz, 2H), 4.06 (d, J=6.9 Hz, 1H), 3.55 (p, J=8.7 Hz, 1H), 3.26 (t, J=7.3 Hz, 2H), 3.18-3.11 (m, 1H), 3.07-2.91 (m, 2H), 2.67 (d, J=3.7 Hz, 1H), 2.52 (dd, J=13.1, 4.4 Hz, 1H), 2.31 (q, J=9.4 Hz, 1H), 2.21 (q, J=8.1, 6.1 Hz, 2H), 2.15-2.00 (m, 3H), 1.93 (d, J=17.7 Hz, 3H), 1.63 (s, 2H), 1.43 (d, J=15.3 Hz, 2H), 1.20 (q, J=7.7 Hz, 2H), 0.75-0.65 (m, 1H), 0.44-0.35 (m, 2H). ¹⁹F NMR (400 MHz, DMSO-d₆): δ 70.186 (s, 1). 74.512 (s, 2).

Compound 675A: LCMS: 547.30 (M+H). Method: Mobile Phase A: Water/0.05% TFA. Mobile Phase B: Acetonitrile/0.05% TFA. Flow rate: 1.5 mL/min. COLUMN: EC-C18 (50×3.0) mm, 1.9μ. Rt (min): 1.379; Area %—98.452. ¹H NMR (400 MHz, DMSO-d₆): δ 11.05 (s, 1H), 9.12 (s, 2H), 7.51 (t, J=7.6 Hz, 1H), 7.41 (dd, J=16.0, 7.6 Hz, 2H), 5.18 (dd, J=13.2, 5.2 Hz, 1H), 4.46-4.36 (m, 3H), 4.16 (s, 1H), 3.63 (p, J=8.7 Hz, 1H), 3.31-3.21 (m, 3H), 2.99 (ddd, J=18.0, 13.5, 5.5 Hz, 1H), 2.67 (d, J=17.3 Hz, 1H), 2.53-2.49 (m, 1H), 2.37-2.22 (m, 3H), 2.09 (dt, J=7.0, 3.5 Hz, 1H), 1.99 (d, J=8.9 Hz, 1H), 1.88 (s, 5H), 1.67 (d, J=42.9 Hz, 3H), 1.24 (q, J=7.2 Hz, 2H), 0.70-0.63 (m, 1H), 0.45-0.36 (m, 2H). ¹⁹F NMR (400 MHz, DMSO-d₆): δ 70.046 (s, 1). 74.306 (s, 2).

### Trifluoroacetate Salt of Compound 678

### 3-(4-((2-cyclopropylethyl)(4-(((1S,2S)-2-(trifluoromethyl)cyclopropyl)amino)cyclohexyl)amino)-1-oxoisoindolin-2-yl)piperidine-2,6-dione Trifluoroacetate

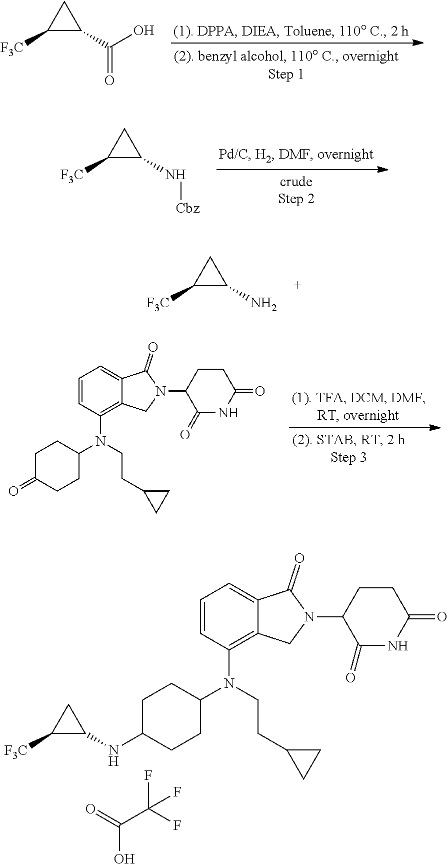

### Step-1: benzyl ((1S,2S)-2-(trifluoromethyl)cyclopropyl)carbamate

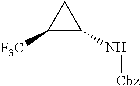 [SMILES: O=C(N[C@H]1C[C@@H]1C(F)(F)F)OCc1ccccc1]

To a stirred solution of (1S,2S)-2-(trifluoromethyl)cyclopropane-1-carboxylic acid (150.0 mg, 0.973 mmol, 1 equiv) and DPPA (321.5 mg, 1.168 mmol, 1.2 equiv) in Toluene (3 mL) was added DIEA (251.6 mg, 1.946 mmol, 2 equiv) at room temperature under nitrogen atmosphere. The resulting mixture was stirred for 2 h at 110° C. under nitrogen atmosphere. To the above mixture was added benzyl alcohol (210.5 mg, 1.946 mmol, 2 equiv) at 50° C. The resulting mixture was stirred for additional overnight at 110° C. The resulting mixture was concentrated under reduced pressure. The residue was purified by reversed-phase flash chromatography with the following conditions: column, C18 silica gel; mobile phase, MeCN in Water (0.1% FA), 40% to 60% gradient in 10 min; detector, UV 254 nm to afford benzyl N-[(1S,2S)-2-(trifluoromethyl)cyclopropyl]carbamate (77.0 mg, 30.51%) as a yellow green oil.

LCMS: 660 [M+H]⁺

### Step-2: (1S,2S)-2-(trifluoromethyl)cyclopropan-1-amine

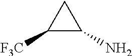

A solution of benzyl N-[(1S,2S)-2-(trifluoromethyl)cyclopropyl]carbamate (177.0 mg, 0.683 mmol, 1 equiv) and Palladium 10% on Carbon (wetted with ca. 55% Water) (174.4 mg, 0.164 mmol, 0.24 equiv) in DMF (3.5 mL) was stirred for overnight at room temperature under hydrogen atmosphere. The resulting mixture was filtered, the filter cake was washed with DMF (1×1 mL). The crude product (dissolved in DMF, 4.5 mL) was used in the next step directly without further purification.

LCMS: 126 [M+H]⁺

### Step-3: 3-(4-((2-cyclopropylethyl)(4-(((1S,2S)-2-(trifluoromethyl)cyclopropyl)amino)cyclohexyl)amino)-1-oxoisoindolin-2-yl)piperidine-2,6-dione trifluoroacetate

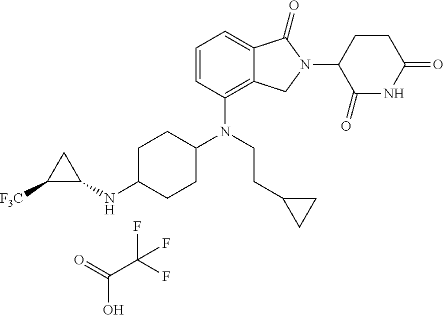

To a stirred solution of (1S,2S)-2-(trifluoromethyl)cyclopropan-1-amine (crude dissolved in DMF, 3.0 mL) and 3-{4-[(2-cyclopropylethyl)(4-oxocyclohexyl)amino]-1-oxo-3H-isoindol-2-yl}piperidine-2,6-dione (80.0 mg, 0.189 mmol, 1 equiv) in DCM (2 mL) was added TFA (64.6 mg, 0.567 mmol, 3 equiv) dropwise at room temperature under nitrogen atmosphere. The resulting mixture was stirred overnight at room temperature under nitrogen atmosphere. To the above mixture was added STAB (200.2 mg, 0.945 mmol, 5 equiv) in portions over 5 min at 0° C. The resulting mixture was stirred for an additional 2 h at room temperature. The resulting mixture was diluted with water (20 mL) and extracted with EtOAc (3×20 mL). The combined organic layers were washed with brine (2×50 mL), dried over anhydrous Na₂SO₄. After filtration, the filtrate was concentrated under reduced pressure. The crude product (20.0 mg) was purified by Prep-HPLC with the following conditions (Column: Sunfire Prep C18 column, 30*150 mm, 5 m; Mobile Phase A: Water (0.05% TFA), Mobile Phase B: ACN; Flow rate: 60 mL/min; Gradient: 10% B to 30% B in 10 min; Wave Length: 254 nm/220 nm; RT1 (min): 11.82/13.53) to afford 3-{4-[(2-cyclopropylethyl)(4-{[(1S,2S)-2-(trifluoromethyl)cyclopropyl]amino}cyclohexyl)amino]-1-oxo-3H-isoindol-2-yl}piperidine-2,6-dione; trifluoroacetic acid (2.9 mg, 1.98% yield) as an off-white solid.

LCMS: 533.3 [M+H]⁺ Method: Mobile Phase A: Water/0.05% TFA. Mobile Phase B: Acetonitrile/0.05% TFA. Flow rate: 1.5 mL/min. COLUMN: SPD-M40 (30×3.0) mm, 1.9μ. Rt (min): 1.360&1.459; Area %—55.33&42.75

¹H-NMR (400 MHz, DMSO-d₆): δ 10.98 (d, J=5.8 Hz, 1H), 9.06 (s, 2H), 7.43 (td, J=7.6, 3.3 Hz, 1H), 7.32 (td, J=13.6, 7.7 Hz, 2H), 5.12 (dt, J=13.4, 5.0 Hz, 1H), 4.44-4.19 (m, 2H), 3.15 (d, J=45.5 Hz, 3H), 2.92 (td, J=17.0, 15.4, 5.4 Hz, 2H), 2.60 (d, J=17.3 Hz, 2H), 2.28 (s, 2H), 2.06 (d, J=43.2 Hz, 2H), 1.81 (d, J=44.7 Hz, 3H), 1.57 (s, 2H), 1.49-1.32 (m, 2H), 1.20 (d, J=32.1 Hz, 3H), 0.85 (d, J=6.9 Hz, 1H), 0.63 (d, J=7.2 Hz, 1H), 0.34 (dt, J=7.9, 2.9 Hz, 2H), −0.06 (d, J=4.8 Hz, 3H).

¹⁹F NMR (377 MHz, DMSO-d₆): δ −64.65 (s,1), −73.67 (s,1).

### Compound 679

### 3-(4-((2-cyclopropylethyl)(4-oxa-1-azaspiro[5.5]undecan-9-yl)amino)-1-oxoisoindolin-2-yl)piperidine-2,6-dione

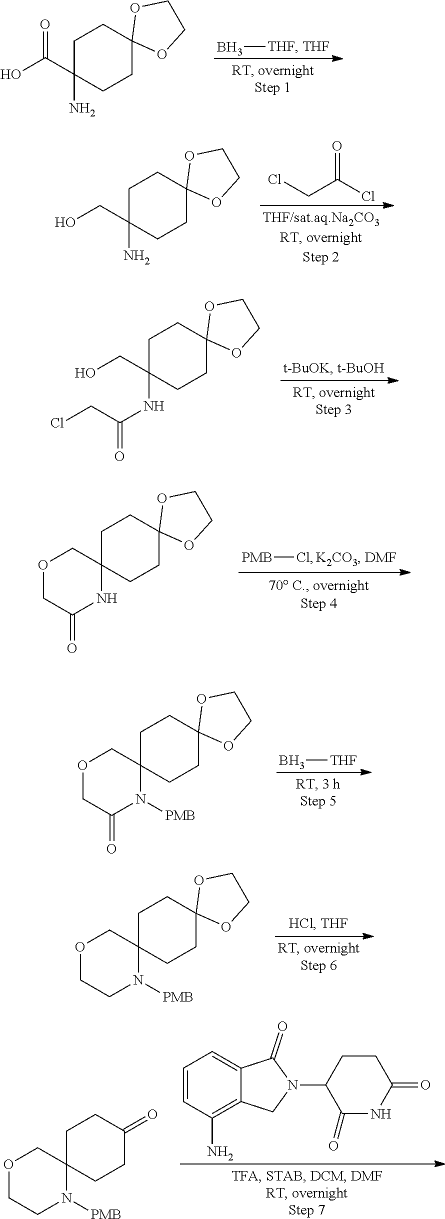

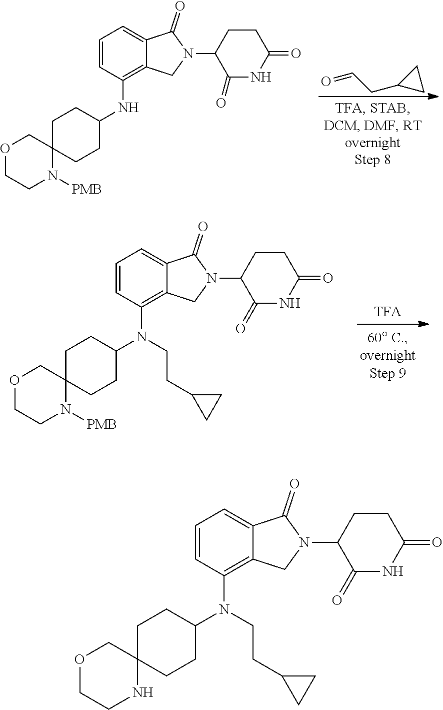

### Step-1: (8-amino-1,4-dioxaspiro[4.5]decan-8-yl)methanol

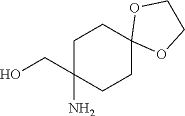 [SMILES: NC1(CO)CCC2(CC1)OCCO2]

To a stirred solution of 8-amino-1,4-dioxaspiro[4.5]decane-8-carboxylic acid (5 g, 24.848 mmol, 1 equiv) in THF (100 mL) was added BH₃-THF (124.2 mL, 124.240 mmol, 5 equiv) dropwise at 0° C. under nitrogen atmosphere. The resulting mixture was stirred for additional overnight at room temperature. The reaction was quenched with MeOH at 0° C. The resulting mixture was diluted with water (100 mL). The resulting mixture was extracted with CH₂Cl₂ (3×50 mL). The combined organic layers were washed with brine (2×100 mL), dried over anhydrous Na₂SO₄. After filtration, the filtrate was concentrated under reduced pressure. The crude product was used in the next step directly without further purification.

LCMS: 188.1 [M+H]⁺

### Step-2: 2-chloro-N-(8-(hydroxymethyl)-1,4-dioxaspiro[4.5]decan-8-yl)acetamide

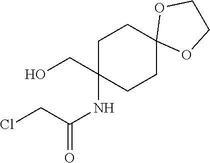 [SMILES: O=C(CCl)NC1(CO)CCC2(CC1)OCCO2]

To a stirred solution of {8-amino-1,4-dioxaspiro[4.5]decan-8-yl}methanol (4.8 g, crude) in THF (48.00 mL) and saturated aqueous Na₂CO₃ (48.00 mL) were added chloroacetyl chloride (14.4 g, 128.180 mmol, 5 equiv) dropwise at 0° C. under nitrogen atmosphere. The resulting mixture was stirred for additional overnight at room temperature. The reaction was quenched by the addition of Water/Ice (5 mL) at 0° C. The resulting mixture was concentrated under reduced pressure. The resulting mixture was diluted with water (100 mL). The resulting mixture was extracted with CH₂Cl₂ (5×100 mL). The combined organic layers were washed with brine (2×200 mL), dried over anhydrous Na₂SO₄. After filtration, the filtrate was concentrated under reduced pressure. The crude product was used in the next step directly without further purification.

LCMS: 264 [M+H]⁺

### Step-3: 1,4,12-trioxa-9-azadispiro[4.2.5⁸.2⁵]pentadecan-10-one

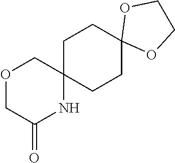 [SMILES: O=C1COCC2(CCC3(CC2)OCCO3)N1]

A solution of 2-chloro-N-[8-(hydroxymethyl)-1,4-dioxaspiro[4.5]decan-8-yl]acetamide (5 g, 18.960 mmol, 1 equiv) and t-BuOK (3.1 g, 28.440 mmol, 1.5 equiv) in t-BuOH (272.73 mL) was stirred for overnight at room temperature under nitrogen atmosphere. The resulting mixture was diluted with water (300 mL). The resulting mixture was extracted with EtOAc (4×100 mL). The combined organic layers were washed with brine (2×300 mL), dried over anhydrous Na₂SO₄. After filtration, the filtrate was concentrated under reduced pressure. The crude product was used in the next step directly without further purification.

LCMS: 228 [M+H]⁺

### Step-4: 9-(4-methoxybenzyl)-1,4,12-trioxa-9-azadispiro[4.2.5⁸.2⁵]pentadecan-10-one

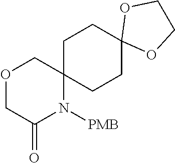

A solution of 1,4,12-trioxa-9-azadispiro[4.2.5⁸.2⁵]pentadecan-10-one (3 g, 13.201 mmol, 1 equiv), PMBCl (4.1 g, 26.402 mmol, 2 equiv) and K₂CO₃ (5.4 g, 39.603 mmol, 3 equiv) in DMF (75.00 mL) was stirred for overnight at 70° C. under nitrogen atmosphere. The mixture was allowed to cool down to room temperature. The resulting mixture was diluted with water (200 mL) and extracted with CH₂Cl₂ (3×150 mL). The combined organic layers were washed with brine (3×200 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 80% gradient in 50 min; detector, UV 254 nm. This resulted in 9-(4-methoxybenzyl)-1,4,12-trioxa-9-azadispiro[4.2.5⁸.2⁵]pentadecan-10-one (200 mg, 4.36%) as a brown oil.

LCMS: 348 [M+H]⁺

### Step-5: 9-(4-methoxybenzyl)-1,4,12-trioxa-9-azadispiro[4.2.5⁸.2⁵]pentadecane

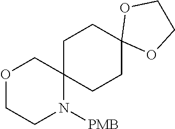

To a stirred solution of 9-(4-methoxybenzyl)-1,4,12-trioxa-9-azadispiro[4.2.5⁸.2⁵]pentadecan-10-one (200 mg, 0.576 mmol, 1 equiv) in THF (2 mL) was added BH₃-THF (1.7 mL, 1.728 mmol, 3 equiv) dropwise at 0° C. under nitrogen atmosphere. The resulting mixture was stirred for an additional 3 h at room temperature. The reaction was quenched by the addition of Water (1 mL) at room temperature. The resulting mixture was concentrated under reduced pressure. The residue was purified by reversed-phase flash chromatography with the following conditions: column, C18 silica gel; mobile phase, MeCN in Water (0.1% FA), 10% to 100% gradient in 15 min; detector, UV 254 nm. This resulted in 9-(4-methoxybenzyl)-1,4,12-trioxa-9-azadispiro[4.2.5⁸.2⁵]pentadecane (70 mg, 37%) as a light brown oil.

LCMS: 334.1 [M+H]⁺

### Step-6: 1-(4-methoxybenzyl)-4-oxa-1-azaspiro[5.5]undecan-9-one

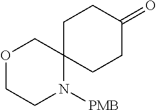

A solution of 9-(4-methoxybenzyl)-1,4,12-trioxa-9-azadispiro[4.2.5⁸.2⁵]pentadecane (100 mg, 0.300 mmol, 1 equiv) in HCl(aq) (2 mL, 2M) and THF (1 mL) was stirred for overnight at room temperature under nitrogen atmosphere. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 1-[(4-methoxyphenyl)methyl]-4-oxa-1-azaspiro[5.5]undecan-9-one (60 mg, 69%) as a light brown oil.

LCMS: 290.1 [M+H]⁺

### Step-7: 3-(4-((1-(4-methoxybenzyl)-4-oxa-1-azaspiro[5.5]undecan-9-yl)amino)-1-oxoisoindolin-2-yl)piperidine-2,6-dione

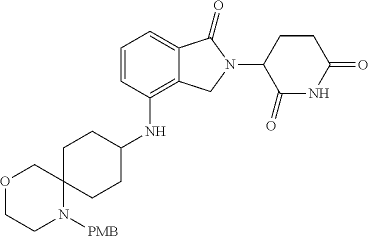

A solution of 1-[(4-methoxyphenyl)methyl]-4-oxa-1-azaspiro[5.5]undecan-9-one (50 mg, 0.173 mmol, 1.00 equiv) in DCM (1 mL) and DMF (0.3 mL) was treated with TFA (0.04 mL, 0.519 mmol, 3 equiv) for overnight at room temperature under nitrogen atmosphere followed by the addition of STAB (183.1 mg, 0.865 mmol, 5 equiv) in portions at 0° C. The resulting mixture was stirred for additional 2 h at room temperature. The resulting mixture was concentrated under reduced pressure. The resulting mixture was filtered, the filter cake was washed with CH₂Cl₂ (3×5 mL). The filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 3-(4-((1-(4-methoxybenzyl)-4-oxa-1-azaspiro[5.5]undecan-9-yl)amino)-1-oxoisoindolin-2-yl)piperidine-2,6-dione (50 mg, 54%) as a light brown oil.

LCMS: 533.3 [M+H]⁺

### Step-8: 3-(4-((2-cyclopropylethyl)(1-(4-methoxybenzyl)-4-oxa-1-azaspiro[5.5]undecan-9-yl)amino)-1-oxoisoindolin-2-yl)piperidine-2,6-dione

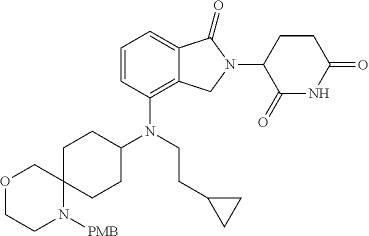

A solution of 3-(4-((1-(4-methoxybenzyl)-4-oxa-1-azaspiro[5.5]undecan-9-yl)amino)-1-oxoisoindolin-2-yl)piperidine-2,6-dione (50 mg, 0.094 mmol, 1 equiv) in DCM (1.5 mL) and DMF (0.3 mL) was treated with TFA (0.02 mL, 0.282 mmol, 3 equiv) for overnight at room temperature under nitrogen atmosphere followed by the addition of STAB (99.4 mg, 0.470 mmol, 5 equiv) dropwise at 0° C. The resulting mixture was stirred for additional 2 h at room temperature. The resulting mixture was diluted with water (10 mL). The resulting mixture was extracted with EtOAc (3×10 mL). The combined organic layers were washed with brine (2×20 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 20% to 70% gradient in 10 min; detector, UV 254 nm. This resulted in 3-(4-((2-cyclopropylethyl)(1-(4-methoxybenzyl)-4-oxa-1-azaspiro[5.5]undecan-9-yl)amino)-1-oxoisoindolin-2-yl)piperidine-2,6-dione (30 mg, 53%) as an off-white solid.

LCMS: 601.4 [M+H]⁺

### Step-9: 3-(4-((2-cyclopropylethyl)(4-oxa-1-azaspiro[5.5]undecan-9-yl)amino)-1-oxoisoindolin-2-yl)piperidine-2,6-dione

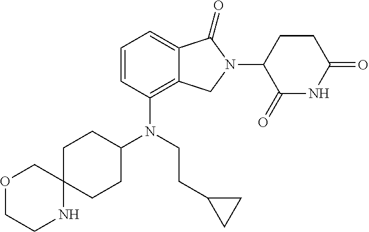 [SMILES: O=C1CCC(N2Cc3c(cccc3N(CCC3CC3)C3CCC4(CC3)COCCN4)C2=O)C(=O)N1]

A solution of 3-(4-((2-cyclopropylethyl)(1-(4-methoxybenzyl)-4-oxa-1-azaspiro[5.5]undecan-9-yl)amino)-1-oxoisoindolin-2-yl)piperidine-2,6-dione (30 mg, 0.050 mmol, 1 equiv) in TFA (0.5 mL) was stirred for overnight at 60° C. under nitrogen atmosphere. The resulting mixture was concentrated under reduced pressure. The crude product (20 mg) was purified by Prep-HPLC with the following conditions (Column: Sunfire Prep C18 column, 30*150 mm, 5 m; Mobile Phase A: Water (0.1% FA), Mobile Phase B: ACN; Flow rate: 60 mL/min mL/min; Gradient: 2% B to 20% B in 10 min; Wave Length: 254 nm/220 nm; RT1 (min): 11.78/12.58) to afford 3-(4-((2-cyclopropylethyl)(4-oxa-1-azaspiro[5.5]undecan-9-yl)amino)-1-oxoisoindolin-2-yl)piperidine-2,6-dione (1.8 mg, 7% yield) as an off-white oil.

LCMS: 481.2 (M+H). Method: Mobile Phase A: Water/0.05% TFA. Mobile Phase B: Acetonitrile/0.05% TFA. Flow rate:1.5 mL/min. COLUMN: Ascentis Express (30×3.0) mm, 2.7μ. Rt (min):0938; Area %—98.968.

¹H NMR (400 MHz, DMSO-d₆): δ 10.97 (s, 1H), 7.41 (t, J=7.6 Hz, 1H), 7.26 (dd, J=19.5, 7.6 Hz, 2H), 5.09 (dd, J=13.1, 5.2 Hz, 1H), 4.36-4.23 (m, 2H), 3.50 (d, J=4.5 Hz, 3H), 3.20 (s, 2H), 2.90 (s, 1H), 2.75 (t, J=4.9 Hz, 2H), 2.61 (s, 1H), 2.12 (s, 1H), 2.01-1.92 (m, 3H), 1.61 (s, 2H), 1.51 (s, 3H), 1.34 (s, 2H), 1.24 (s, 3H), 1.17-1.11 (m, 3H), 0.85 (t, J=6.4 Hz, 1H), 0.36-0.27 (m, 2H), −0.08 (dd, J=5.6, 4.1 Hz, 2H).

### Compounds 680 and 680A

### 3-(4-[(2-cyclopropylethyl)[(1r,4r)-4-[(2-methoxyethyl)(methyl)amino]cyclohexyl]amino]-1-oxo-3H-isoindol-2-ylpiperidine-2,6-dione; trifluoroacetic acid and 3-(4-[(2-cyclopropylethyl)[(1s,4s)-4-[(2-methoxyethyl)(methyl)amino]cyclohexyl]amino]-1-oxo-3H-isoindol-2-ylpiperidine-2,6-dione; trifluoroacetic acid

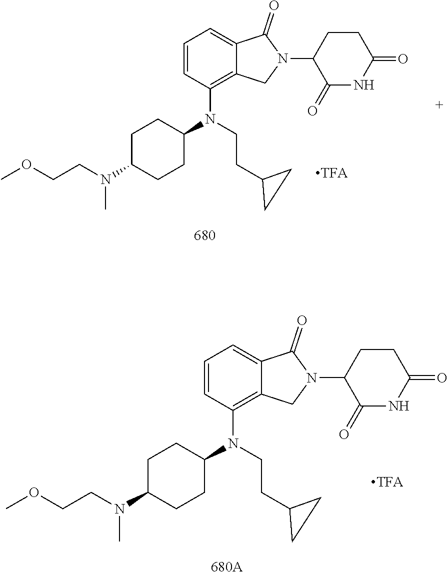 [SMILES: COCCN(C)[C@H]1CC[C@H](N(CCC2CC2)c2cccc3c2CN(C2CCC(=O)NC2=O)C3=O)CC1 ; COCCN(C)[C@H]1CC[C@@H](N(CCC2CC2)c2cccc3c2CN(C2CCC(=O)NC2=O)C3=O)CC1]

The compounds were prepared by the general procedure described in the preparation of Compounds 515 and 515A using the appropriate amine.

Compound 680: LC-MS: (ES, m/z)=497.20 (M+H). Method: Mobile phase A:Water/5 mM NH4HCO3 Mobile phase B:Acetonitrile. Flow rate: 1.2000 mL/min Column Name: EVO C18 (50×3.0) mm, 2.6 μm. Rt (min): 1.597; Area %—95.184. ¹H NMR (400 MHz, DMSO-d₆): ¹H NMR (400 MHz, DMSO-d₆) δ 11.02 (s, 1H), 9.27 (s, 1H), 7.48 (t, J=7.6 Hz, 1H), 7.35 (dd, J=15.4, 7.6 Hz, 2H), 5.16 (dd, J=13.3, 5.1 Hz, 1H), 4.42 (d, J=17.2 Hz, 1H), 4.30 (d, J=17.1 Hz, 1H), 3.70 (t, J=5.1 Hz, 2H), 3.34-3.13 (m, 6H), 2.98 (td, J=13.0, 6.9 Hz, 1H), 2.76 (d, J=4.8 Hz, 3H), 2.66 (d, J=17.5 Hz, 1H), 2.51 (d, J=9.1 Hz, 1H), 2.14-1.85 (m, 6H), 1.72-1.55 (m, 4H), 1.20 (q, J=7.0 Hz, 2H), 0.69 (s, 1H), 0.40 (dd, J=8.5, 4.6 Hz, 2H). ¹⁹F NMR (376 MHz, DMSO-d₆) δ −73.69 (s,1).

Compound 680A: LC-MS: (ES, m/z)=497.35 (M+H). Method: Mobile phase A: Water/5 mM NH₄HCO₃ Mobile phase B:Acetonitrile. Flow rate: 1.2000 mL/min Column Name: EVO C18 (50×3.0) mm, 2.6 μm. Rt (min): 3.74; Area %—99.318. ¹H NMR (400 MHz, DMSO-d₆): δ 11.08 (s, 1H), 9.41 (s, 1H), 7.60-7.46 (m, 3H), 5.23 (ddd, J=13.3, 5.2, 3.1 Hz, 1H), 4.51-4.37 (m, 2H), 3.75 (t, J=5.1 Hz, 2H), 3.62 (s, 1H), 3.32-3.14 (m, 3H), 3.02 (ddd, J=18.0, 13.5, 5.3 Hz, 1H), 2.82 (t, J=4.2 Hz, 3H), 2.73-2.62 (m, 1H), 2.18-2.07 (m, 1H), 1.99 (d, J=13.7 Hz, 2H), 1.87-1.69 (m, 4H), 1.59 (d, J=12.9 Hz, 2H), 1.30 (h, J=6.9, 6.0 Hz, 2H), 0.62 (dt, J=7.8, 4.7 Hz, 1H), 0.41 (dt, J=8.2, 2.8 Hz, 2H), 0.06 (s, 2H). ¹⁹F NMR (376 MHz, DMSO-d₆) δ −74.3 (s,1).

### Compounds 681 and 681A

### 3-{4-[(2-cyclopropylethyl)[(1r,4r)-4-[methyl(4,4,4-trifluorobutyl)amino]cyclohexyl]amino]-1-oxo-3H-isoindol-2-yl}piperidine-2,6-dione and 3-{4-[(2-cyclopropylethyl)[(1s,4s)-4-[methyl(4,4,4-trifluorobutyl)amino]cyclohexyl]amino]-1-oxo-3H-isoindol-2-yl}piperidine-2,6-dione

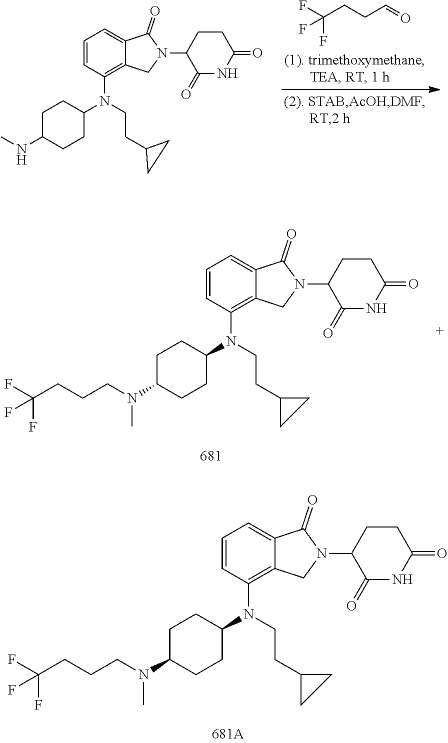 [SMILES: CNC1CCC(N(CCC2CC2)c2cccc3c2CN(C2CCC(=O)NC2=O)C3=O)CC1 ; O=CCCC(F)(F)F ; CN(CCCC(F)(F)F)[C@H]1CC[C@H](N(CCC2CC2)c2cccc3c2CN(C2CCC(=O)NC2=O)C3=O)CC1 ; CN(CCCC(F)(F)F)[C@H]1CC[C@@H](N(CCC2CC2)c2cccc3c2CN(C2CCC(=O)NC2=O)C3=O)CC1]

To a stirred solution of 3-{4-[(2-cyclopropylethyl)[4-(methylamino)cyclohexyl]amino]-1-oxo-3H-isoindol-2-yl}piperidine-2,6-dione (150 mg, 0.342 mmol, 1 equiv) and 4,4,4-trifluorobutanal (431 mg, 3.42 mmol, 10 equiv) in trimethyl orthoformate (3 mL) was added TEA (52 mg, 0.51 mmol, 1.5 equiv) at room temperature under nitrogen atmosphere. The resulting mixture was stirred for 1 h at room temperature under nitrogen atmosphere. To the above mixture was added AcOH (103 mg, 1.7 mmol, 5 equiv) and STAB (362 mg, 1.7 mmol, 5 equiv) in DMF (1.5 mL) dropwise over 5 min at 0° C. The resulting mixture was stirred for additional 2 h at room temperature. The resulting mixture was diluted with water (50 mL) extracted with EtOAc (3×25 mL). The combined organic layers were washed with brine (2×50 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 30% to 40% gradient in 10 min; detector, UV 254 nm to afford 180 mg crude. The crude product (180 mg) was purified by Prep-HPLC with the following conditions (Column: Xselect CSH F-Phenyl OBD column 30*250 mm, 5 μm; Mobile Phase A: Water (0.10% FA), Mobile Phase B: ACN; Flow rate: 60 mL/min; Gradient: 2% B to 19% B in 10 min; Wave Length: 254 nm/220 nm; RT1 (min): 11.62/13.33) to afford 3-{4-[(2-cyclopropylethyl)[(1r,4r)-4-[methyl(4,4,4-trifluorobutyl)amino]cyclohexyl]amino]-1-oxo-3H-isoindol-2-yl}piperidine-2,6-dione (681) (31 mg, purity: 98%, yield: 16%) as an off-white solid and 3-{4-[(2-cyclopropylethyl)[(1s,4s)-4-[methyl(4,4,4-trifluorobutyl)amino]cyclohexyl]amino]-1-oxo-3H-isoindol-2-yl}piperidine-2,6-dione (681A) (33 mg, purity: 98%, yield: 17%) as an off-white solid.

Compound 681: LCMS: 549.40 (M+H). Method: Mobile Phase A: Water/0.04% NH₃H₂O. Mobile Phase B: Acetonitrile. Flow rate: 1.5 mL/min. COLUMN: kinetex EVO C18 (50×3.0) mm, 2.6μ. Rt (min): 1.830; Area %—98.22. ¹H NMR (400 MHz, DMSO-d₆): δ 11.04 (s, 1H), 7.48 (t, J=7.7 Hz, 1H), 7.34 (dd, J=16.0, 7.6 Hz, 2H), 5.15 (dd, J=13.1, 5.1 Hz, 1H), 4.41 (d, J=17.2 Hz, 1H), 4.31 (d, J=17.2 Hz, 1H), 3.25 (hept, J=7.0 Hz, 4H), 3.14 (tt, J=11.6, 3.3 Hz, 1H), 2.97 (ddd, J=17.2, 13.5, 5.4 Hz, 1H), 2.79 (q, J=11.8, 7.5 Hz, 3H), 2.71-2.60 (m, 1H), 2.44 (s, 2H), 2.33 (dddd, J=20.1, 16.1, 9.7, 5.4 Hz, 2H), 2.13-2.02 (m, 1H), 1.98-1.85 (m, 3H), 1.77 (q, J=7.8 Hz, 2H), 1.65 (h, J=11.2 Hz, 2H), 1.50 (d, J=11.8 Hz, 1H), 1.47-1.41 (m, 1H), 1.20 (q, J=7.0 Hz, 2H), 0.69 (pd, J=7.2, 3.6 Hz, 1H), 0.44-0.33 (m, 2H), 0.06 (s, 2H).

Compound 681A: LCMS: 549.40 (M+H). Method: Mobile Phase A: Water/0.04% NH₃H₂O. Mobile Phase B: Acetonitrile. Flow rate: 1.5 mL/min. COLUMN: kinetex EVO C18 (50×3.0) mm, 2.6μ. Rt (min): 1.974; Area %—98.46. ¹H NMR (400 MHz, DMSO-d6): δ 11.05 (s, 1H), 7.50 (t, J=7.6 Hz, 1H), 7.39 (dd, J=17.6, 7.6 Hz, 2H), 5.18 (dd, J=13.2, 5.1 Hz, 1H), 4.45 (d, J=17.2 Hz, 1H), 4.35 (d, J=17.2 Hz, 1H), 3.43 (dt, J=8.0, 4.2 Hz, 2H), 3.24 (p, J=6.5 Hz, 3H), 2.98 (ddd, J=18.0, 13.6, 5.4 Hz, 1H), 2.71-2.62 (m, 3H), 2.62-2.48 (m, 5H), 2.09 (ddd, J=10.6, 6.2, 3.9 Hz, 1H), 1.96-1.80 (m, 4H), 1.75 (p, J=7.6 Hz, 2H), 1.49 (q, J=12.2 Hz, 4H), 1.34-1.19 (m, 2H), 0.66 (dq, J=12.6, 5.8, 5.3 Hz, 1H), 0.40 (dd, J=8.6, 4.2 Hz, 2H), 0.06 (d, J=8.7 Hz, 2H).

### Compounds 682 and 682A

### 3-(4-[(2-cyclopropylethyl)[(1r,4r)-4-[methyl(3,3,3-trifluoropropyl)amino]cyclohexyl]amino]-1-oxo-3H-isoindol-2-ylpiperidine-2,6-dione; trifluoroacetic acid and 3-(4-[(2-cyclopropylethyl)[(1s,4s)-4-[methyl(3,3,3-trifluoropropyl)amino]cyclohexyl]amino]-1-oxo-3H-isoindol-2-ylpiperidine-2,6-dione; trifluoroacetic acid

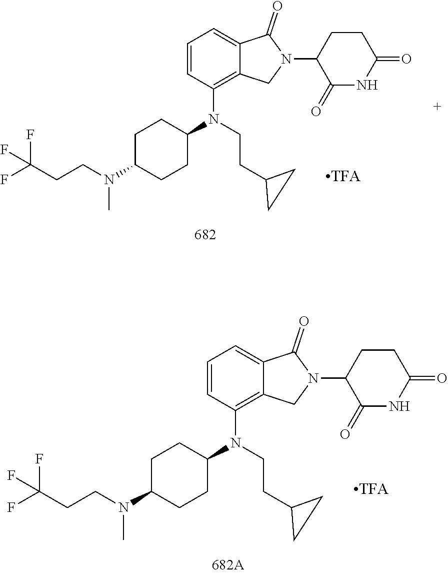 [SMILES: CN(CCC(F)(F)F)[C@H]1CC[C@H](N(CCC2CC2)c2cccc3c2CN(C2CCC(=O)NC2=O)C3=O)CC1 ; CN(CCC(F)(F)F)[C@H]1CC[C@@H](N(CCC2CC2)c2cccc3c2CN(C2CCC(=O)NC2=O)C3=O)CC1]

The compounds were prepared by the same procedure described in the preparation of Compounds 681 and 681A above using the appropriate aldehyde.

Compound 682: LC-MS: (ES, m/z): [M+1]=535.15 (M+H). Method: Mobile phase A:Water/5 mM NH₄HCO₃ Mobile phase B:Acetonitrile. Flow rate: 1.2000 mL/min Column Name: EVO C18 (50×3.0) mm, 2.6 μm. Rt (min): 1.761; Area %—95.164. ¹H NMR (400 MHz, DMSO-d₆): δ 11.02 (s, 1H), 9.82 (s, 1H), 7.48 (t, J=7.7 Hz, 1H), 7.36 (dd, J=13.6, 7.6 Hz, 2H), 5.15 (dd, J=13.3, 5.0 Hz, 1H), 4.42 (d, J=17.1 Hz, 1H), 4.33 (s, 1H), 3.24 (dp, J=19.1, 6.3, 5.9 Hz, 3H), 3.05-2.85 (m, 3H), 2.82-2.76 (m, 3H), 2.67 (t, J=12.1 Hz, 1H), 2.12-1.89 (m, 5H), 1.67 (d, J=11.9 Hz, 4H), 1.20 (q, J 7.0 Hz, 2H), 0.70 (t, J=7.0 Hz, 1H), 0.40 (dt, J=8.1, 2.7 Hz, 2H. ¹⁹F NMR (376 MHz, DMSO-d₆) δ −63.79 (s,1), −74.12 (s,1).

Compound 682A: LC-MS: (ES, m/z): [M+1]=515.30 (M+H). Method: Mobile phase A:Water/5 mM NH₄HCO₃ Mobile phase B:Acetonitrile. Flow rate: 1.2000 mL/min Column Name: EVO C18 (50×3.0) mm, 2.6 μm. Rt (min): 1.889; Area %—95.098. ¹H NMR (400 MHz, DMSO-d₆): δ 10.98 (d, J=3.9 Hz, 1H), 9.66 (s, 1H), 7.51-7.34 (m, 3H), 5.14 (dd, J=13.2, 5.2 Hz, 1H), 4.37 (dd, J=21.6, 16.5 Hz, 2H), 3.54 (s, 1H), 3.11 (s, 2H), 2.97-2.85 (m, 2H), 2.76 (d, J=4.3 Hz, 3H), 2.64-2.55 (m, 1H), 2.43 (dd, J=13.2, 4.4 Hz, 1H), 2.04 (q, J=6.8, 5.9 Hz, 1H), 1.93-1.73 (m, 3H), 1.65 (s, 3H), 1.50 (s, 2H), 1.21 (dd, J=13.8, 6.7 Hz, 2H), 0.59-0.51 (m, 1H), 0.33 (dd, J=8.0, 1.7 Hz, 2H), −0.08 (d, J=4.9 Hz, 2H). ¹⁹F NMR (376 MHz, DMSO-d₆) δ −63.75 (s,1), −74.12 (s,1).

### Trifluoroacetate Salt Forms of Compound 683 and 683A

### 3-(4-((2-cyclopropylethyl)((1r,4r)-4-((3,3-difluoropropyl)(methyl)amino)cyclohexyl)amino)-1-oxoisoindolin-2-yl)piperidine-2,6-dione tris(2,2,2-trifluoroacetate) (683) and 3-(4-((2-cyclopropylethyl)((1s,4s)-4-((3,3-difluoropropyl)(methyl)amino)cyclohexyl)amino)-1-oxoisoindolin-2-yl)piperidine-2,6-dione bis(2,2,2-trifluoroacetate) (683A)

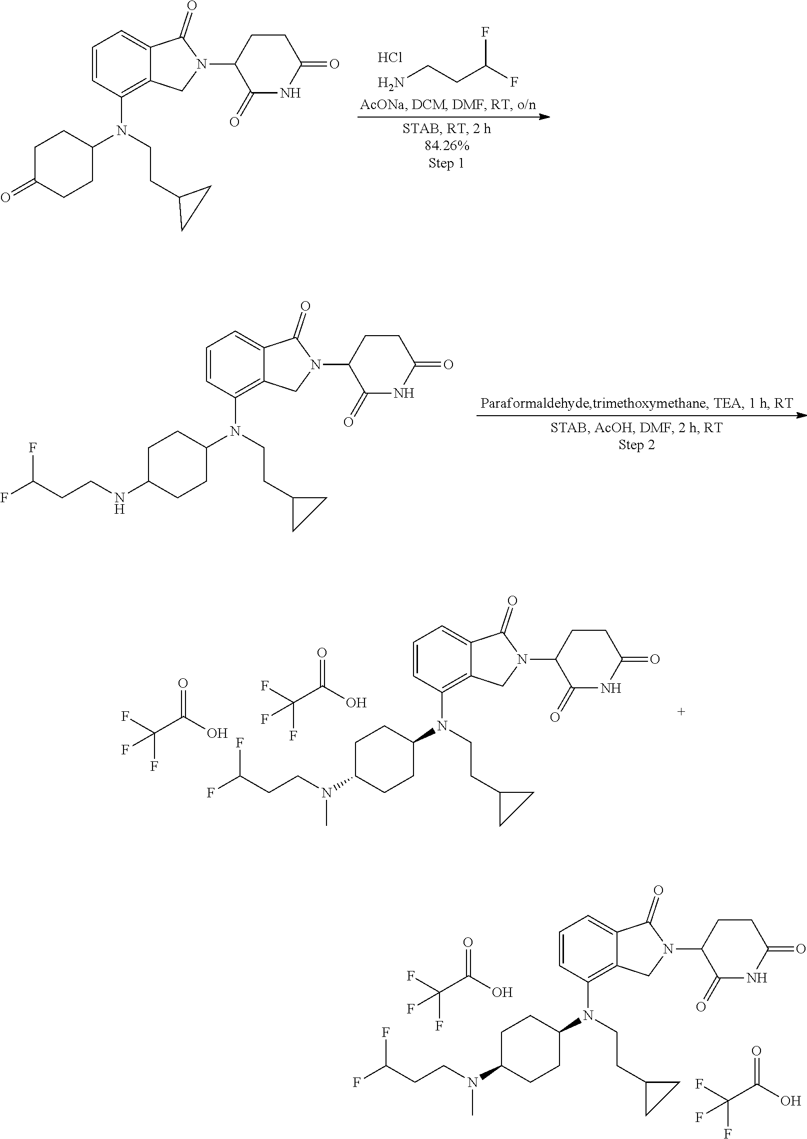

### Step-1: 3-(4-((2-cyclopropylethyl)(4-((3,3-difluoropropyl)amino)cyclohexyl)amino)-1-oxoisoindolin-2-yl)piperidine-2,6-dione

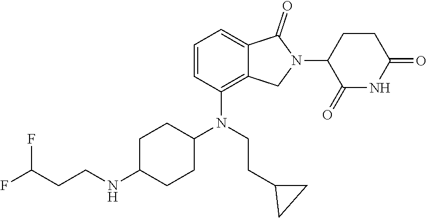 [SMILES: O=C1CCC(N2Cc3c(cccc3N(CCC3CC3)C3CCC(NCCC(F)F)CC3)C2=O)C(=O)N1]

To a stirred solution of 3-(4-((2-cyclopropylethyl)(4-oxocyclohexyl)amino)-1-oxoisoindolin-2-yl)piperidine-2,6-dione (500.0 mg, 1.181 mmol, 1 equiv) and 3,3-difluoropropan-1-amine hydrochloride (465.9 mg, 3.543 mmol, 3 equiv) in DCM (12.5 mL) and DMF (3 mL) was added AcONa (484.2 mg, 5.905 mmol, 5 equiv) at room temperature under nitrogen atmosphere. The resulting mixture was stirred overnight at room temperature under nitrogen atmosphere. To the above mixture was added STAB (1.3 g, 5.905 mmol, 5 equiv) in portions over 5 min at 0° C. The resulting mixture was stirred for an additional 2 h at room temperature. The resulting mixture was diluted with water (100 mL). The resulting mixture was extracted with EtOAc (3×200 mL). The combined organic layers were washed with brine (3×100 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.10% FA), 10% to 20% gradient in 5 min; detector, UV 254 nm to afford 3-(4-((2-cyclopropylethyl)(4-((3,3-difluoropropyl)amino)cyclohexyl)amino)-1-oxoisoindolin-2-yl)piperidine-2,6-dione (500.0 mg, 84%) as a light pink solid.

LCMS: 503.2 [M+H]⁺

### Step-2: 3-(4-((2-cyclopropylethyl)((1r,4r)-4-((3,3-difluoropropyl)(methyl)amino)cyclohexyl)amino)-1-oxoisoindolin-2-yl)piperidine-2,6-dione tris(2,2,2-trifluoroacetate) and 3-(4-((2-cyclopropylethyl)((1s,4s)-4-((3,3-difluoropropyl)(methyl)amino)cyclohexyl)amino)-1-oxoisoindolin-2-yl)piperidine-2,6-dione bis(2,2,2-trifluoroacetate)

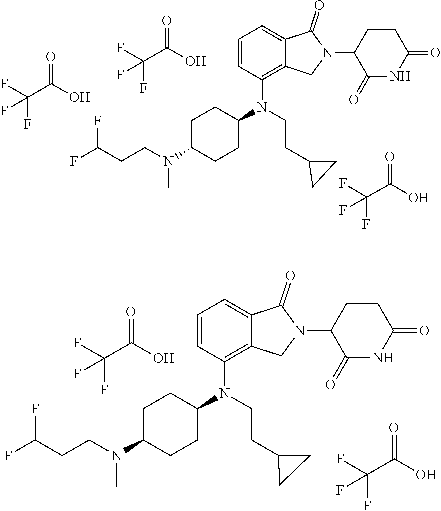

To a stirred solution of 3-{4-[(2-cyclopropylethyl)({4-[(3,3-difluoropropyl)amino]cyclohexyl})amino]-1-oxo-3H-isoindol-2-yl}piperidine-2,6-dione (200.0 mg, 0.398 mmol, 1 equiv) and Paraformalclehyde (87.6 mg, 1.990 mmol, 5 equiv) in trimethoxymethane (2 mL) was added TEA (60.4 mg, 0.597 mmol, 1.5 equiv) at room temperature under nitrogen atmosphere. The resulting mixture was stirred for 1 h at room temperature under nitrogen atmosphere. To the above mixture was added AcOH (119.4 mg, 1.990 mmol, 5 equiv) and STAB (421.6 mg, 1.990 mmol, 5 equiv) in DMF (4 mL) dropwise over 2 min at −10° C. The resulting mixture was stirred for an additional 2 h at room temperature. The resulting mixture was diluted with water (50 mL). The resulting mixture was extracted with CH₂Cl₂ (3×50 mL). The combined organic layers were washed with brine (3×100 mL), dried over anhydrous Na₂SO₄. After filtration, the filtrate was concentrated under reduced pressure. The crude product (150.0 mg) was purified by Prep-HPLC with the following conditions (Column: XBridge Prep Phenyl OBD Column 19*250 mm, 5 m; Mobile Phase A: Water (0.05% TFA), Mobile Phase B: ACN; Flow rate: 60 mL/min; Gradient: 2% B to 10% B in 10 min; Wave Length: 254 nm/220 nm; RT1 (min): 5.23) to afford 3-(4-((2-cyclopropylethyl)((1r,4r)-4-((3,3-difluoropropyl)(methyl)amino)cyclohexyl)amino)-1-oxoisoindolin-2-yl)piperidine-2,6-dione tris(2,2,2-trifluoroacetate) (27.5 mg, 11%) as an off-white solid and 3-(4-((2-cyclopropylethyl)((1s,4s)-4-((3,3-difluoropropyl)(methyl)amino)cyclohexyl)amino)-1-oxoisoindolin-2-yl)piperidine-2,6-dione tris(2,2,2-trifluoroacetate) (50.0 mg, crude) as an off-white solid. The crude product (50.0 mg) was purified again via Prep-HPLC with the following conditions (Column: XBridge Prep Phenyl OBD Column 19*250 mm, 5 m; Mobile Phase A: Water (0.05% TFA), Mobile Phase B: ACN; Flow rate: 60 mL/min; Gradient: 2% B to 10% B in 15 min; Wave Length: 254 nm/220 nm; RT1 (min): 5.23) to afford 3-(4-((2-cyclopropylethyl)((1s,4s)-4-((3,3-difluoropropyl)(methyl)amino)cyclohexyl)amino)-1-oxoisoindolin-2-yl)piperidine-2,6-dione bis(2,2,2-trifluoroacetate) (22.6 mg, 9%) as an off-white solid.

### 3-(4-((2-cyclopropylethyl)((1r,4r)-4-((3,3-difluoropropyl)(methyl)amino)cyclohexyl)amino)-1-oxoisoindolin-2-yl)piperidine-2,6-dione tris(2,2,2-trifluoroacetate)

LCMS: 517.3 (M+H). Method: Mobile Phase A: Water/0.05% TFA. Mobile Phase B: Acetonitrile/0.05% TFA. Flow rate: 1.5 mL/min. COLUMN: EC-C18 (30×3.0) mm, 1.9μ. Rt (min): 1.259; Area %—99.61.

¹H NMR (400 MHz, DMSO-d₆): δ 11.05 (s, 1H), 9.81 (s, 1H), 7.59-7.22 (m, 3H), 6.27 (tt, J=56.0, 4.2 Hz, 1H), 5.16 (dd, J=13.2, 5.3 Hz, 1H), 4.54-4.20 (m, 2H), 3.26 (ddt, J=31.8, 11.5, 6.3 Hz, 6H), 2.97 (ddd, J=18.0, 13.5, 5.4 Hz, 1H), 2.77 (d, J=4.5 Hz, 3H), 2.71-2.61 (m, 1H), 2.53 (d, J=5.4 Hz, 1H), 2.38 (th, J=14.2, 4.5 Hz, 2H), 2.16-1.83 (m, 5H), 1.67 (dp, J=27.1, 12.9 Hz, 4H), 1.19 (q, J=7.0 Hz, 2H), 0.70 (p, J=6.4 Hz, 1H), 0.39 (d, J=7.9 Hz, 2H), 0.08-−0.11 (m, 2H).

¹⁹F NMR (377 MHz, DMSO-d6): δ −74.7 (s,5), −116.2 (s,1).

### 3-(4-((2-cyclopropylethyl)((1s,4s)-4-((3,3-difluoropropyl)(methyl)amino)cyclohexyl)amino)-1-oxoisoindolin-2-yl)piperidine-2,6-dione bis(2,2,2-trifluoroacetate)

LCMS: 517.3 (M+H). Method: Mobile Phase A: Water/0.05% TFA. Mobile Phase B: Acetonitrile/0.05% TFA. Flow rate: 1.5 mL/min. COLUMN: EC-C18 (30×3.0) mm, 1.9μ. Rt (min): 1.444; Area %—99.70.

¹H NMR (400 MHz, DMSO-d₆): δ 11.09 (d, J=3.1 Hz, 1H), 9.80 (s, 1H), 7.63-7.24 (m, 3H), 6.29 (tt, J=55.6, 4.3 Hz, 1H), 5.32-5.04 (m, 1H), 4.57-4.29 (m, 2H), 3.61 (s, 1H), 3.40 (t, J=11.9 Hz, 2H), 3.21 (th, J=19.8, 6.1 Hz, 3H), 3.02 (ddd, J=18.0, 13.5, 5.4 Hz, 1H), 2.84 (t, J=4.5 Hz, 3H), 2.74-2.62 (m, 1H), 2.55-2.24 (m, 3H), 2.21-2.06 (m, 1H), 1.97 (s, 2H), 1.89-1.66 (m, 4H), 1.65-1.45 (m, 2H), 1.27 (td, J=13.9, 6.6 Hz, 2H), 0.62 (dp, J=12.7, 5.9 Hz, 1H), 0.48-0.31 (m, 2H), 0.11-−0.12 (m, 2H).

¹⁹F NMR (377 MHz, DMSO-d6): δ −74.4 (s,3.5), −116.1 (s,1).

### Compound 703

### 3-{4-[(2-cyclopropylethyl)(3-{[(3,3,3-trifluoropropyl)amino]methyl}bicyclo[1.1.1]pentan-1-yl)amino]-1-oxo-3H-isoindol-2-yl}piperidine-2,6-dione

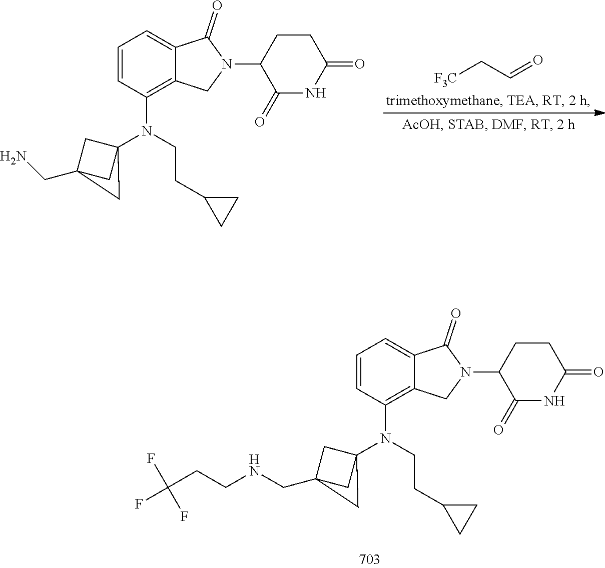 [SMILES: NCC12CC(N(CCC3CC3)c3cccc4c3CN(C3CCC(=O)NC3=O)C4=O)(C1)C2 ; O=CCC(F)(F)F ; O=C1CCC(N2Cc3c(cccc3N(CCC3CC3)C34CC(CNCCC(F)(F)F)(C3)C4)C2=O)C(=O)N1]

To a stirred solution of 3-(4-{[3-(aminomethyl)bicyclo[1.1.1]pentan-1-yl](2-cyclopropylethyl)amino}-1-oxo-3H-isoindol-2-yl)piperidine-2,6-dione (150 mg, 0.35 mmol, 1 equiv) and 3,3,3-trifluoropropanal (79 mg, 0.71 mmol, 2 equiv) in trimethoxymethane (3 mL) was added TEA (54 mg, 0.53 mmol, 1.5 equiv) at room temperature under nitrogen atmosphere. The resulting mixture was stirred for 2 h at room temperature under nitrogen atmosphere. To the above mixture were added STAB (376 mg, 1.7 mmol, 5 equiv) and AcOH (106 mg, 1.7 mmol, 5 equiv) in DMF (1.5 mL) dropwise over 3 min at 0° C. The resulting mixture was stirred for additional 2 h at room temperature. The resulting mixture was diluted with water (50 mL). The resulting mixture was extracted with EtOAc (3×50 mL). The combined organic layers were washed with brine (3×100 mL), dried over anhydrous Na₂SO₄. After filtration, the filtrate was concentrated under reduced pressure. The crude product (100 mg) was purified by Prep-HPLC with the following conditions Column: (Xselect CSH C18 OBD Column 30*150 mm 5 μm, n; Mobile Phase A: Water (0.1% FA), Mobile Phase B: ACN; Flow rate: 60 mL/min; Gradient: 5% B to 25% B in 8 min; Wave Length: 254 nm/220 nm; RT1 (min): 9.5) to afford 3-{4-[(2-cyclopropylethyl)(3-{[(3,3,3-trifluoropropyl)amino]methyl}bicyclo[1.1.1]pentan-1-yl)amino]-1-oxo-3H-isoindol-2-yl}piperidine-2,6-dione (14 mg, 8%) as an off-white solid.

Compound 703: LCMS: 519.3 (M+H). Method: Mobile Phase A: Water/0.05% TFA. Mobile Phase B: Acetonitrile/0.05% TFA. Flow rate: 1.5 mL/min. COLUMN: EC-C18 (30×3.0) mm, 1.9μ. Rt (min): 1.282; Area %—97.13. ¹H NMR (300 MHz, DMSO-d6): δ 11.04 (s, 1H), 7.59-7.40 (m, 3H), 5.17 (dd, J=13.1, 5.1 Hz, 1H), 4.46-4.27 (m, 2H), 3.23 (t, J=7.4 Hz, 2H), 2.97 (ddd, J=17.4, 13.3, 5.2 Hz, 1H), 2.84-2.65 (m, 4H), 2.61 (d, J=4.5 Hz, 1H), 2.53-2.31 (m, 3H), 2.13-1.97 (m, 1H), 1.76 (s, 6H), 1.38-1.12 (m, 3H), 0.79-0.57 (m, 1H), 0.45-0.32 (m, 2H), 0.07-−0.06 (m, 2H).

### Compound 708 and 708A

### 3-(4-((2-cyclopropylethyl)((1s,4s)-4-(((3,3,3-trifluoropropyl)amino)methyl)cyclohexyl)amino)-1-oxoisoindolin-2-yl)piperidine-2,6-dione (708A) and 3-(4-((2-cyclopropylethyl)((1r,4r)-4-(((3,3,3-trifluoropropyl)amino)methyl)cyclohexyl)amino)-1-oxoisoindolin-2-yl)piperidine-2,6-dione (708)

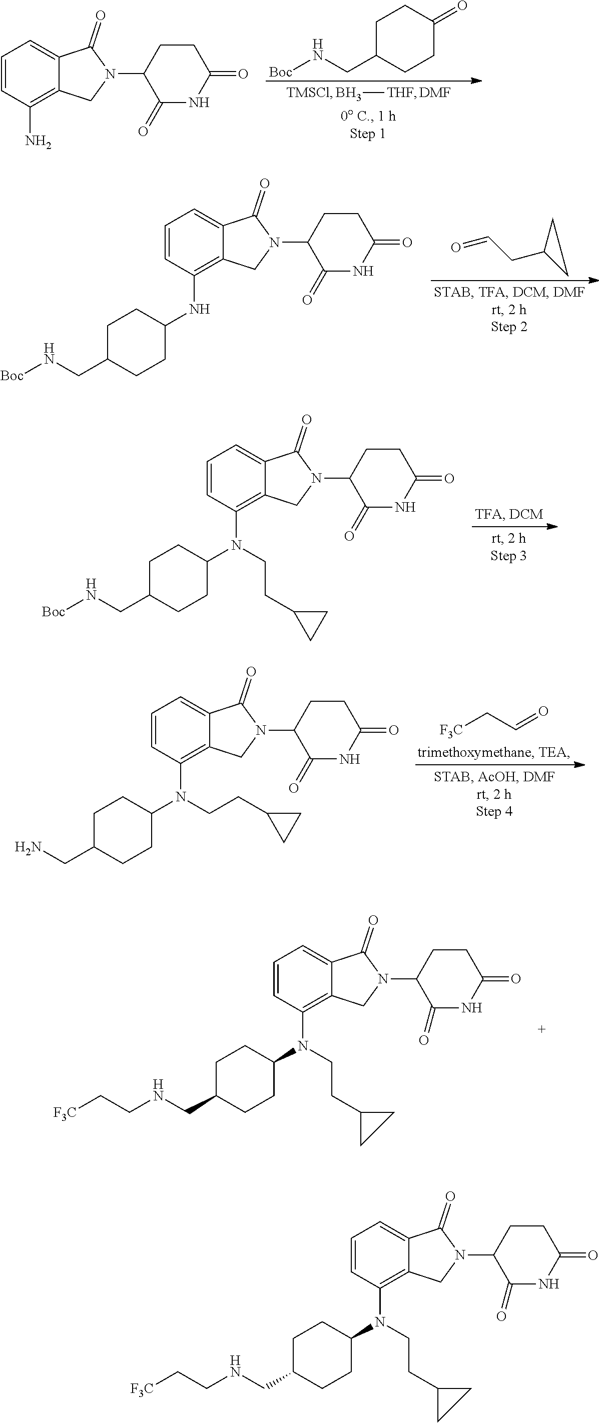

### Step-1: tert-butyl ((4-((2-(2,6-dioxopiperidin-3-yl)-1-oxoisoindolin-4-yl)amino)cyclohexyl)methyl)carbamate

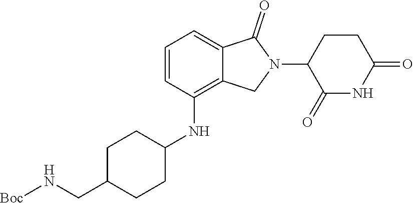 [SMILES: CC(C)(C)OC(=O)NCC1CCC(Nc2cccc3c2CN(C2CCC(=O)NC2=O)C3=O)CC1]

A solution of lenalidomide (1.37 g, 5.279 mmol, 1.2 equiv) and tert-butyl N-[(4-oxocyclohexyl)methyl]carbamate (1 g, 4.399 mmol, 1.00 equiv) in DMF (15 mL) was treated with TMSCl (4.78 g, 43.990 mmol, 10 equiv) for 1 h at 0° C. under nitrogen atmosphere followed by the addition of BH₃-THF (22 mL, 22.9 mmol, 5 equiv) dropwise at 0° C. The resulting mixture was stirred for 1 h at 0° C. under nitrogen atmosphere. The reaction was quenched with Water/Ice at 0° C. The resulting mixture was extracted with EtOAc (3×50 mL). The combined organic layers were washed with brine (3×100 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 30% to 60% gradient in 15 min; detector, UV 254 nm. This resulted in tert-butyl ((4-((2-(2,6-dioxopiperidin-3-yl)-1-oxoisoindolin-4-yl)amino)cyclohexyl)methyl)carbamate (2 g, 97%) as a brown solid.

LCMS: 415.2 [M+H]⁺

### Step-2: tert-butyl ((4-((2-cyclopropylethyl)(2-(2,6-dioxopiperidin-3-yl)-1-oxoisoindolin-4-yl)amino)cyclohexyl)methyl)carbamate

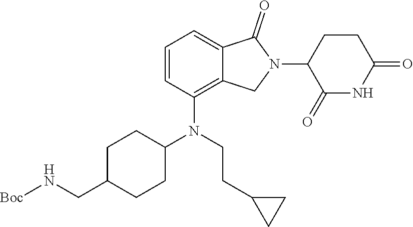 [SMILES: CC(C)(C)OC(=O)NCC1CCC(N(CCC2CC2)c2cccc3c2CN(C2CCC(=O)NC2=O)C3=O)CC1]

A solution of tert-butyl ((4-((2-(2,6-dioxopiperidin-3-yl)-1-oxoisoindolin-4-yl)amino)cyclohexyl)methyl)carbamate (1.5 g, 3.188 mmol, 1 equiv) and 2-cyclopropylacetaldehyde (5.36 g, 63.760 mmol, 20 equiv) in DCM/DMF (40 ml/9.6 ml) was treated with TFA (1.09 g, 9.564 mmol, 3 equiv) for overnight at room temperature under nitrogen atmosphere followed by the addition of STAB (3.38 g, 15.940 mmol, 5 equiv) in portions at 0° C. The resulting mixture was stirred for 2 h at room temperature under nitrogen atmosphere. The resulting mixture was filtered, the filter cake was washed with DCM (3×5 mL). The filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 220 nm. This resulted in tert-butyl ((4-((2-cyclopropylethyl)(2-(2,6-dioxopiperidin-3-yl)-1-oxoisoindolin-4-yl)amino)cyclohexyl)methyl)carbamate (1.3 g, 76%) as a yellow oil.

LCMS: 539.35 [M+H]⁺

### Step-3: 3-(4-((4-(aminomethyl)cyclohexyl)(2-cyclopropylethyl)amino)-1-oxoisoindolin-2-yl)piperidine-2,6-dione

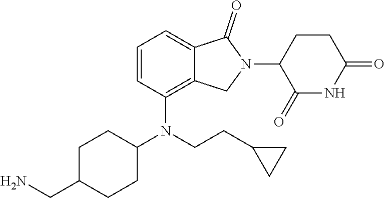 [SMILES: NCC1CCC(N(CCC2CC2)c2cccc3c2CN(C2CCC(=O)NC2=O)C3=O)CC1]

To a stirred mixture of tert-butyl ((4-((2-cyclopropylethyl)(2-(2,6-dioxopiperidin-3-yl)-1-oxoisoindolin-4-yl)amino)cyclohexyl)methyl)carbamate (1.3 g, 2.413 mmol, 1 equiv) in DCM (12.5 ml) was added TFA (2.5 mL) dropwise at 0° C. under air atmosphere. The resulting mixture was stirred for 2 h at room temperature under the air atmosphere. The resulting mixture was concentrated under vacuum. This resulted in 3-(4-((4-(aminomethyl)cyclohexyl)(2-cyclopropylethyl)amino)-1-oxoisoindolin-2-yl)piperidine-2,6-dione (1 g, 94%) as a brown oil.

LCMS: 439.4 [M+H]⁺

### Step-4:3-(4-((2-cyclopropylethyl)((1s,4s)-4-(((3,3,3-trifluoropropyl)amino)methyl)cyclohexyl)amino)-1-oxoisoindolin-2-yl)piperidine-2,6-dione and 3-(4-((2-cyclopropylethyl)((1r,4r)-4-(((3,3,3-trifluoropropyl)amino)methyl)cyclohexyl)amino)-1-oxoisoindolin-2-yl)piperidine-2,6-dione

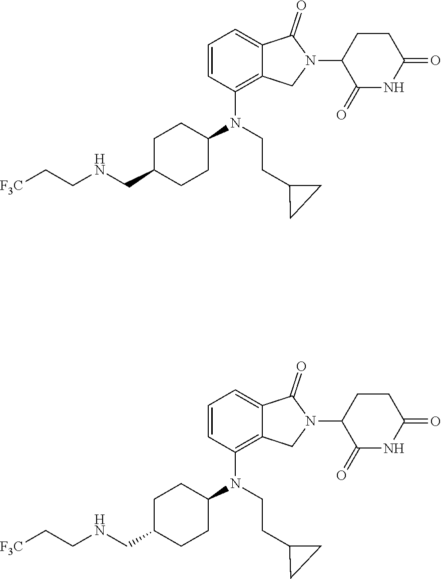 [SMILES: O=C1CCC(N2Cc3c(cccc3N(CCC3CC3)[C@H]3CC[C@@H](CNCCC(F)(F)F)CC3)C2=O)C(=O)N1 ; O=C1CCC(N2Cc3c(cccc3N(CCC3CC3)[C@H]3CC[C@H](CNCCC(F)(F)F)CC3)C2=O)C(=O)N1]

A solution of 3-(4-((4-(aminomethyl)cyclohexyl)(2-cyclopropylethyl)amino)-1-oxoisoindolin-2-yl)piperidine-2,6-dione (1 g, 2.280 mmol, 1 equiv) and 3,3,3-trifluoropropanal (255.5 mg, 2.280 mmol, 1 equiv) in DCM/DMF (25 ml/6 ml) was treated with TFA (780.0 mg, 6.840 mmol, 3 equiv) for 2 h at room temperature under nitrogen atmosphere followed by the addition of STAB (2.42 g, 11.400 mmol, 5 equiv) in portions at 0° C. The resulting mixture was stirred for 2 h at room temperature under nitrogen atmosphere. The resulting mixture was filtered, the filter cake was washed with DCM (3×5 mL). The filtrate was concentrated under reduced pressure. The crude product was purified by Prep-HPLC with the following conditions (Column: XBridge Prep Phenyl OBD Column 19*250 mm, 5 m; Mobile Phase A: Water (0.1% FA), Mobile Phase B: ACN; Flow rate: 60 mL/min mL/min; Gradient: 17% B to 32% B in 10 min; Wave Length: 254 nm/220 nm; RT1 (min): 7+8.44) to afford 3-(4-((2-cyclopropylethyl)((1s,4s)-4-(((3,3,3-trifluoropropyl)amino)methyl)cyclohexyl)amino)-1-oxoisoindolin-2-yl)piperidine-2,6-dione (10.3 mg, 0.80%) and 3-(4-((2-cyclopropylethyl)((1r,4r)-4-(((3,3,3-trifluoropropyl)amino)methyl)cyclohexyl)amino)-1-oxoisoindolin-2-yl)piperidine-2,6-dione (5.9 mg, 0.48%) both all off-white solid.

### 3-(4-((2-cyclopropylethyl)((1r,4r)-4-(((3,3,3-trifluoropropyl)amino)methyl)cyclohexyl)amino)-1-oxoisoindolin-2-yl)piperidine-2,6-dione

LCMS: (ES, m/z): [M+1]=535.25

¹H NMR (400 MHz, DMSO-d₆) δ 10.96 (s, 1H), 7.41 (t, J=7.6 Hz, 1H), 7.33-7.22 (m, 2H), 5.09 (dd, J=13.1, 5.1 Hz, 1H), 4.30 (q, J=17.2 Hz, 2H), 3.26-3.11 (m, 3H), 2.90 (ddd, J=17.0, 13.5, 5.4 Hz, 1H), 2.73 (dd, J=8.1, 6.8 Hz, 2H), 2.64-2.55 (m, 2H), 2.39 (tdd, J=11.8, 7.2, 3.9 Hz, 2H), 2.06-1.95 (m, 1H), 1.61 (s, 4H), 1.54-1.36 (m, 4H), 1.25-1.04 (m, 3H), 0.61 (q, J=8.6, 6.9 Hz, 1H), 0.40-0.26 (m, 2H), −0.04-−0.12 (m, 2H).

Method: Mobile Phase A: Water/0.1% FA, Mobile Phase B: Acetonitrile/0.1% FA. Flow rate:1.5000 mL/min. COLUMN: EC-C18 (30×3.0) mm, 1.9μ, Rt (min): 1.287; Area %—99.870.

### 3-(4-((2-cyclopropylethyl)((1s,4s)-4-(((3,3,3-trifluoropropyl)amino)methyl)cyclohexyl)amino)-1-oxoisoindolin-2-yl)piperidine-2,6-dione

LCMS: (ES, m/z): [M+1]=533.30

¹H NMR (400 MHz, DMSO-d₆) δ 10.96 (s, 1H), 7.40 (t, J=7.6 Hz, 1H), 7.24 (dd, J=16.8, 7.6 Hz, 2H), 5.09 (dd, J=13.1, 5.1 Hz, 1H), 4.42-4.16 (m, 2H), 3.21 (dd, J=8.7, 6.0 Hz, 2H), 3.11-3.00 (m, 1H), 2.89 (ddd, J=17.8, 13.4, 5.4 Hz, 1H), 2.68 (t, J=7.4 Hz, 2H), 2.57 (t, J=3.1 Hz, 1H), 2.43-2.30 (m, 4H), 2.08-1.96 (m, 1H), 1.78 (t, J=13.4 Hz, 4H), 1.49 (s, 2H), 1.31 (s, 1H), 1.14 (q, J=7.1 Hz, 2H), 1.03-0.82 (m, 2H), 0.63 (q, J=6.4, 6.0 Hz, 1H), 0.37-0.26 (m, 2H), −0.03-−0.11 (m, 2H).

Method: Mobile Phase A: Water/0.1% FA, Mobile Phase B: Acetonitrile/0.1% FA. Flow rate:1.5000 mL/min. COLUMN:EC-C18 (30×3.0) mm, 1.9μ, Rt (min): 1.228; Area %—99.600.

### Trifluoroacetate Salt Forms of Compound 711 and 711A

### 3-(4-((2-cyclopropylethyl)((1r,4r)-4-(1,1-difluoro-5-azaspiro[2.4]heptan-5-yl)cyclohexyl)amino)-1-oxoisoindolin-2-yl)piperidine-2,6-dione 2,2,2-trifluoroacetate (711) and 3-(4-((2-cyclopropylethyl)((1s,4s)-4-(1,1-difluoro-5-azaspiro[2.4]heptan-5-yl)cyclohexyl)amino)-1-oxoisoindolin-2-yl)piperidine-2,6-dione 2,2,2-trifluoroacetate (711A)

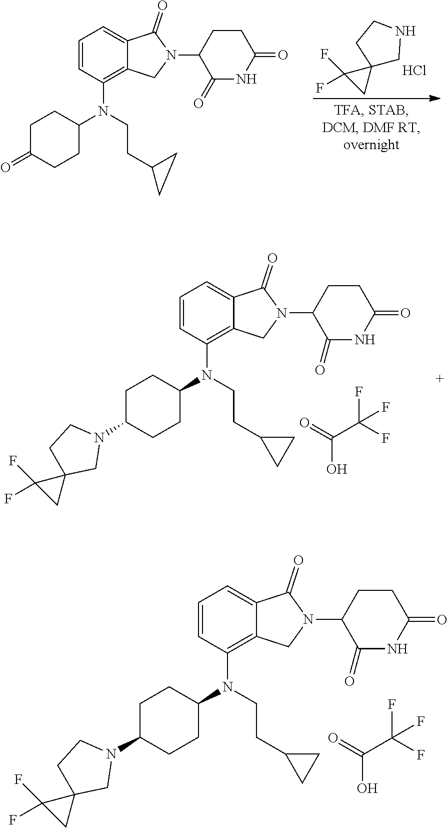

A solution of 3-{4-[(2-cyclopropylethyl)(4-oxocyclohexyl)amino]-1-oxo-3H-isoindol-2-yl}piperidine-2,6-dione (150 mg, 0.354 mmol, 1.00 equiv) in DCM (3 mL)/DMF (1 mL) was treated with TFA (201.9 mg, 1.770 mmol, 5 equiv) for overnight at room temperature under nitrogen atmosphere followed by the addition of STAB (375.3 mg, 1.770 mmol, 5 equiv) dropwise at 0° C. The resulting mixture was stirred for additional 2 h at room temperature. The resulting mixture was diluted with water and extracted with CH₂Cl₂ (3×20 mL). The combined organic layers were washed with brine (2×25 mL), dried over anhydrous Na₂SO₄. The crude product (140 mg) was purified by Prep-HPLC with the following conditions (Column: Xselect CSH C18 OBD Column 30*150 mm 5 μm, n; Mobile Phase A: Water (0.05% TFA), Mobile Phase B: ACN; Flow rate: 60 mL/min mL/min; Gradient: 6% B to 26% B in 8 min; Wave Length: 254 nm/220 nm; RT1 (min): 9.57/11.50) to afford 3-(4-((2-cyclopropylethyl)((1r,4r)-4-(1,1-difluoro-5-azaspiro[2.4]heptan-5-yl)cyclohexyl)amino)-1-oxoisoindolin-2-yl)piperidine-2,6-dione 2,2,2-trifluoroacetate (56 mg, 29% yield) as an off-white solid and 3-(4-((2-cyclopropylethyl)((1s,4s)-4-(1,1-difluoro-5-azaspiro[2.4]heptan-5-yl)cyclohexyl)amino)-1-oxoisoindolin-2-yl)piperidine-2,6-dione 2,2,2-trifluoroacetate (42 mg, 21% yield) as an off-white solid.

### 3-(4-((2-cyclopropylethyl)((1r,4r)-4-(1,1-difluoro-5-azaspiro[2.4]heptan-5-yl)cyclohexyl)amino)-1-oxoisoindolin-2-yl)piperidine-2,6-dione 2,2,2-trifluoroacetate

LCMS: 541.50 (M+H). Method: Mobile Phase A: Water/0.04% NH₃H₂O. Mobile Phase B: Acetonitrile. Flow rate:1.5 mL/min. COLUMN: EVO-C18 (50×3.0) mm, 2.6μ. Rt (min): 1.889; Area %—99.218.

¹H NMR (400 MHz, DMSO-d₆):δ 10.97 (s, 1H), 10.23 (d, J=68.8 Hz, 1H), 7.43 (t, J=7.7 Hz, 1H), 7.31 (dd, J=13.0, 7.6 Hz, 2H), 5.12 (dd, J=13.2, 5.1 Hz, 1H), 4.39-4.23 (m, 2H), 3.72 (s, 1H), 3.59 (s, 1H), 3.48-3.37 (m, 2H), 3.17 (dt, J=23.1, 8.4 Hz, 4H), 2.92 (ddd, J=17.2, 13.5, 5.4 Hz, 1H), 2.64-2.55 (m, 1H), 2.47 (d, J=4.4 Hz, 1H), 2.18-1.69 (m, 9H), 1.52 (dt, J=22.5, 11.8 Hz, 4H), 1.14 (q, J=7.1 Hz, 2H), 0.71-0.57 (m, 1H), 0.38-0.29 (m, 2H), −0.06 (dd, J=5.0, 1.5 Hz, 2H).

¹⁹F NMR (400 MHz, DMSO-d₆): δ −74.274 (s, 26), −136.717˜136.984 (m, 14).

### 3-(4-((2-cyclopropylethyl)((1s,4s)-4-(1,1-difluoro-5-azaspiro[2.4]heptan-5-yl)cyclohexyl)amino)-1-oxoisoindolin-2-yl)piperidine-2,6-dione 2,2,2-trifluoroacetate

LCMS: 541.45 (M+H). Method: Mobile Phase A: Water/0.04% NH₃H₂O. Mobile Phase B: Acetonitrile. Flow rate:1.5 mL/min. COLUMN: EVO-C18 (50×3.0) mm, 2.6μ. Rt (min):1.922; Area %—98.270.

¹H NMR (400 MHz, DMSO-d₆):δ 10.99 (s, 1H), 10.06 (d, J=77.7 Hz, 1H), 7.45 (t, J=7.6 Hz, 1H), 7.41-7.30 (m, 2H), 5.16-5.06 (m, 1H), 4.38 (s, 1H), 4.31 (s, 1H), 3.85 (s, 1H), 3.72 (s, 1H), 3.64-3.56 (m, 1H), 3.32 (d, J=29.7 Hz, 4H), 3.18 (p, J=7.7, 7.2 Hz, 2H), 2.95-2.88 (m, 1H), 2.62 (s, 1H), 2.43 (d, J=9.0 Hz, 1H), 2.26 (d, J=13.4 Hz, 1H), 2.13 (s, 1H), 2.07-1.99 (m, 1H), 1.92-1.71 (m, 8H), 1.55 (s, 2H), 1.30-1.22 (m, 1H), 1.17 (q, J=7.4 Hz, 2H), 0.63-0.54 (m, 1H), 0.34 (dt, J=8.0, 2.8 Hz, 2H), −0.08 (q, J=4.6 Hz, 2H).

¹⁹F NMR (400 MHz, DMSO-d₆): δ −74.201 (s, 23), −134.807˜136.196 (m, 11).

### Trifluoroacetate Salt Forms of Compound 712 and 712A

### 3-(4-((2-cyclopropylethyl)((1S,4r)-4-((S)-3-(trifluoromethyl)pyrrolidin-1-yl)cyclohexyl)amino)-1-oxoisoindolin-2-yl)piperidine-2,6-dione 2,2,2-trifluoroacetate (712) and 3-(4-((2-cyclopropylethyl)((1R,4s)-4-((S)-3-(trifluoromethyl)pyrrolidin-1-yl)cyclohexyl)amino)-1-oxoisoindolin-2-yl)piperidine-2,6-dione 2,2,2-trifluoroacetate (712A)

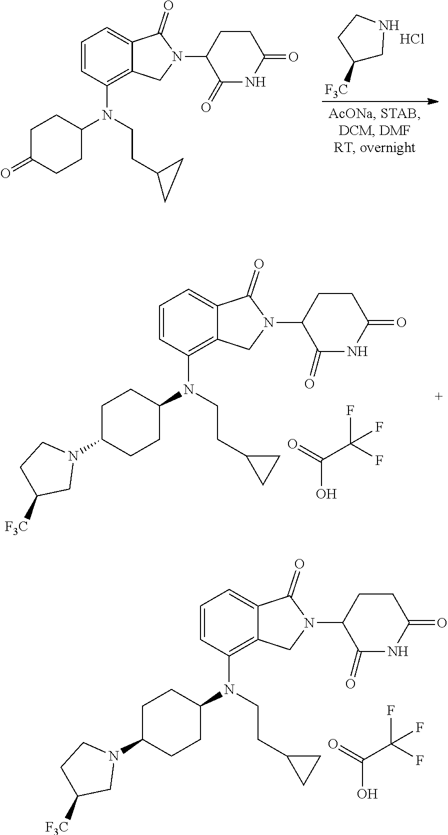

A solution of 3-{4-[(2-cyclopropylethyl)(4-oxocyclohexyl)amino]-1-oxo-3H-isoindol-2-yl}piperidine-2,6-dione (145 mg, 0.342 mmol, 1 equiv) in DCM (4 mL)/DMF (1 mL) was treated with AcONa (140.4 mg, 1.710 mmol, 5 equiv) for overnight at room temperature under nitrogen atmosphere followed by the addition of STAB (362.8 mg, 1.710 mmol, 5 equiv) dropwise at 0° C. The resulting mixture was stirred for an additional 2 h at room temperature. The resulting mixture was diluted with water and extracted with CH₂Cl₂ (3×15 mL). The combined organic layers were washed with brine (2×30 mL), dried over anhydrous Na₂SO₄. The crude product (120 mg) was purified by Prep-HPLC with the following conditions (Column: Sunfire Prep C18 column, 30*150 mm, 5 m; Mobile Phase A: Water (0.05% TFA), Mobile Phase B: ACN; Flow rate: 60 mL/min mL/min; Gradient: 10% B to 28% B in 10 min; Wave Length: 254 nm/220 nm; RT1 (min): 11.23/12.95) to afford 3-(4-((2-cyclopropylethyl)((1S,4r)-4-((S)-3-(trifluoromethyl)pyrrolidin-1-yl)cyclohexyl)amino)-1-oxoisoindolin-2-yl)piperidine-2,6-dione 2,2,2-trifluoroacetate (34.4 mg, 18% yield) as an off-white solid and to afford 3-(4-((2-cyclopropylethyl)((1R,4s)-4-((S)-3-(trifluoromethyl)pyrrolidin-1-yl)cyclohexyl)amino)-1-oxoisoindolin-2-yl)piperidine-2,6-dione 2,2,2-trifluoroacetate (30 mg, 16% yield) as an off-white solid.

### 3-(4-((2-cyclopropylethyl)((1S,4r)-4-((S)-3-(trifluoromethyl)pyrrolidin-1-yl)cyclohexyl)amino)-1-oxoisoindolin-2-yl)piperidine-2,6-dione 2,2,2-trifluoroacetate

LCMS: 547.20 (M+H). Method: Mobile Phase A: Water/5 mM NH4HCO3. Mobile Phase B: Acetonitrile. Flow rate:1.5 mL/min. COLUMN: Poroshell HPH-C18 (50x 3.0) mm, 4μ. Rt (min): 1.671; Area %—99.531.

¹H NMR (400 MHz, DMSO-d₆): δ 10.98 (s, 1H), 10.05 (d, J=43.6 Hz, 1H), 7.49-7.30 (m, 3H), 5.11 (dd, J=13.2, 5.1 Hz, 1H), 4.42-4.29 (m, 2H), 4.02 (s, 1H), 3.77 (s, 3H), 3.37 (s, 2H), 3.17 (t, J=7.1 Hz, 4H), 2.99-2.85 (m, 1H), 2.60 (d, J=17.3 Hz, 1H), 2.45-2.39 (m, 1H), 2.31-2.17 (m, 1H), 2.07-1.99 (m, 1H), 1.95-1.73 (m, 6H), 1.53 (s, 2H), 1.17 (d, J=7.4 Hz, 2H), 0.59 (s, 1H), 0.33 (dt, J=8.0, 2.7 Hz, 2H), −0.08 (t, J=4.6 Hz, 2H).

¹⁹F NMR (400 MHz, DMSO-d₆): δ −74.014 (s, 2), −69.234˜−69.629 (m, 2).

### 3-(4-((2-cyclopropylethyl)((1R,4s)-4-((S)-3-(trifluoromethyl)pyrrolidin-1-yl)cyclohexyl)amino)-1-oxoisoindolin-2-yl)piperidine-2,6-dione 2,2,2-trifluoroacetate

LCMS: 547.25 (M+H). Method: Mobile Phase A: Water/5 mM NH4HCO3. Mobile Phase B: Acetonitrile. Flow rate:1.5 mL/min. COLUMN: Poroshell HPH-C18 (50×3.0) mm, 4μ. Rt (min):1.901; Area %—99.712.

¹H NMR (400 MHz, DMSO-d₆): δ 10.96 (s, 1H), 10.24 (d, J=20.8 Hz, 1H), 7.37 (dd, J=38.7, 7.5 Hz, 3H), 5.12 (dd, J=13.2, 5.1 Hz, 1H), 4.35 (d, J=17.0 Hz, 2H), 3.39 (s, 1H), 3.20 (s, 5H), 2.92 (t, J=14.0 Hz, 1H), 2.62 (s, 1H), 2.14 (s, 3H), 2.00 (s, 1H), 1.87 (s, 2H), 1.51 (s, 4H), 1.18-1.13 (m, 2H), 0.64 (s, 1H), 0.33 (q, J=5.2 Hz, 2H), −0.06 (d, J=4.8 Hz, 2H).

¹⁹F NMR (400 MHz, DMSO-d₆): δ −74.251 (s, 1), −69.619˜−69.673 (m, 1).

### Trifluoroacetate Salt Forms of Compound 713 and 713A

### 3-(4-((2-cyclopropylethyl)((1R,4r)-4-((R)-3-(trifluoromethyl)pyrrolidin-1-yl)cyclohexyl)amino)-1-oxoisoindolin-2-yl)piperidine-2,6-dione bis(2,2,2-trifluoroacetate) (713) and 3-(4-((2-cyclopropylethyl)((1S,4s)-4-((R)-3-(trifluoromethyl)pyrrolidin-1-yl)cyclohexyl)amino)-1-oxoisoindolin-2-yl)piperidine-2,6-dione bis(2,2,2-trifluoroacetate) (713A)

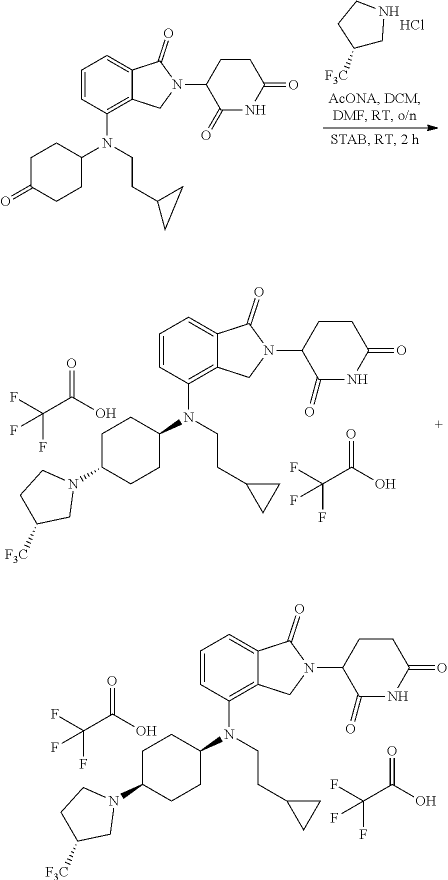

To a stirred solution of 3-{4-[(2-cyclopropylethyl)(4-oxocyclohexyl)amino]-1-oxo-3H-isoindol-2-yl}piperidine-2,6-dione (150.0 mg, 0.354 mmol, 1 equiv) and (3R)-3-(trifluoromethyl)pyrrolidine hydrochloride (93.3 mg, 0.531 mmol, 1.5 equiv) in DCM (3.75 mL) and DMF (0.9 mL) was added AcONa (145.3 mg, 1.770 mmol, 5 equiv) at room temperature under nitrogen atmosphere. The resulting mixture was stirred for overnight at room temperature under nitrogen atmosphere. To the above mixture was added STAB (375.3 mg, 1.770 mmol, 5 equiv) in portions over 3 min at 0° C. The resulting mixture was stirred for additional 2 h at room temperature. The resulting mixture was diluted with water (30 mL). The resulting mixture was extracted with EtOAc (3×20 mL). The combined organic layers were washed with brine (3×50 mL), dried over anhydrous Na₂SO₄. After filtration, the filtrate was concentrated under reduced pressure. The crude product (140.0 mg) was purified by Prep-HPLC with the following conditions (Column: XBridge Prep Phenyl OBD Column 19*250 mm, 5 m; Mobile Phase A: Water (0.05% TFA), Mobile Phase B: ACN; Flow rate: 60 mL/min; Gradient: 20% B to 35% B in 10 min; Wave Length: 254 nm/220 nm; RT1 (min): 8.45+11.24) to afford 3-(4-((2-cyclopropylethyl)((1R,4r)-4-((R)-3-(trifluoromethyl)pyrrolidin-1-yl)cyclohexyl)amino)-1-oxoisoindolin-2-yl)piperidine-2,6-dione bis(2,2,2-trifluoroacetate) (64 mg, 27%) as an off-white solid and 3-(4-((2-cyclopropylethyl)((1S,4s)-4-((R)-3-(trifluoromethyl)pyrrolidin-1-yl)cyclohexyl)amino)-1-oxoisoindolin-2-yl)piperidine-2,6-dione bis(2,2,2-trifluoroacetate) (50 mg, 21%) as an off-white solid.

### 3-(4-((2-cyclopropylethyl)((1R,4r)-4-((R)-3-(trifluoromethyl)pyrrolidin-1-yl)cyclohexyl)amino)-1-oxoisoindolin-2-yl)piperidine-2,6-dione bis(2,2,2-trifluoroacetate)

LCMS: 547.3 (M+H). Method: Mobile Phase A: Water/0.05% TFA. Mobile Phase B: Acetonitrile/0.05% TFA. Flow rate: 1.5 mL/min. COLUMN: EC-C18 (30×3.0) mm, 1.9μ. Rt (min): 1.186; Area %—97.07.

¹H NMR (400 MHz, DMSO-d₆): δ 10.96 (s, 1H), 10.42 (s, 1H), 7.43 (q, J=6.7, 5.7 Hz, 1H), 7.32 (dd, J=12.8, 7.5 Hz, 2H), 5.12 (dd, J=13.2, 5.2 Hz, 1H), 4.45-4.16 (m, 2H), 3.96-3.33 (m, 3H), 3.30-3.02 (m, 5H), 2.92 (ddd, J=17.9, 13.5, 5.4 Hz, 1H), 2.65-2.53 (m, 1H), 2.48-2.30 (m, 1H), 2.14 (d, J=17.0 Hz, 3H), 2.05-1.96 (m, 1H), 1.86 (d, J=11.4 Hz, 2H), 1.53 (d, J=16.9 Hz, 4H), 1.15 (q, J=7.1 Hz, 2H), 0.63 (h, J=6.6, 5.9 Hz, 1H), 0.33 (dt, J=8.6, 2.7 Hz, 2H), −0.07 (t, J=4.9 Hz, 2H).

¹⁹F NMR (377 MHz, DMSO-d₆): δ −69.6 (s,1), −74.4 (s,2).

### 3-(4-((2-cyclopropylethyl)((1S,4s)-4-((R)-3-(trifluoromethyl)pyrrolidin-1-yl)cyclohexyl)amino)-1-oxoisoindolin-2-yl)piperidine-2,6-dione bis(2,2,2-trifluoroacetate)

LCMS: 547.3 (M+H). Method: Mobile Phase A: Water/0.05% TFA. Mobile Phase B: Acetonitrile/0.05% TFA. Flow rate: 1.5 mL/min. COLUMN: EC-C18 (30×3.0) mm, 1.9μ. Rt (min): 1.308; Area %—99.42.

¹H NMR (400 MHz, DMSO-d₆): δ 10.97 (s, 1H), 10.40 (s, 1H), 7.44 (t, J=7.6 Hz, 1H), 7.32 (dd, J=12.6, 7.6 Hz, 2H), 5.12 (dd, J=13.2, 5.1 Hz, 1H), 4.45-4.14 (m, 2H), 3.90 (s, 1H), 3.78-3.30 (m, 4H), 3.19 (td, J=21.0, 18.6, 12.1 Hz, 6H), 2.92 (ddd, J=17.9, 13.6, 5.4 Hz, 1H), 2.67-2.55 (m, 1H), 2.48-2.29 (m, 1H), 2.24-2.06 (m, 3H), 2.00 (t, J=6.0, 3.7 Hz, 1H), 1.86 (d, J=12.8 Hz, 3H), 1.54 (dd, J=21.9, 11.3 Hz, 4H), 1.14 (q, J=7.1 Hz, 2H), 0.64 (ddd, J=12.6, 7.8, 4.9 Hz, 1H), 0.33 (dt, J=8.5, 2.8 Hz, 2H), −0.06 (q, J=4.9 Hz, 2H).

¹⁹F NMR (377 MHz, DMSO-d₆): δ −69.6 (s,1), −74.4 (s,2).

### Compound 714

### 3-(4-((2-cyclopropylethyl)((1-methylpiperidin-4-yl)methyl)amino)-1-oxoisoindolin-2-yl)piperidine-2,6-dione

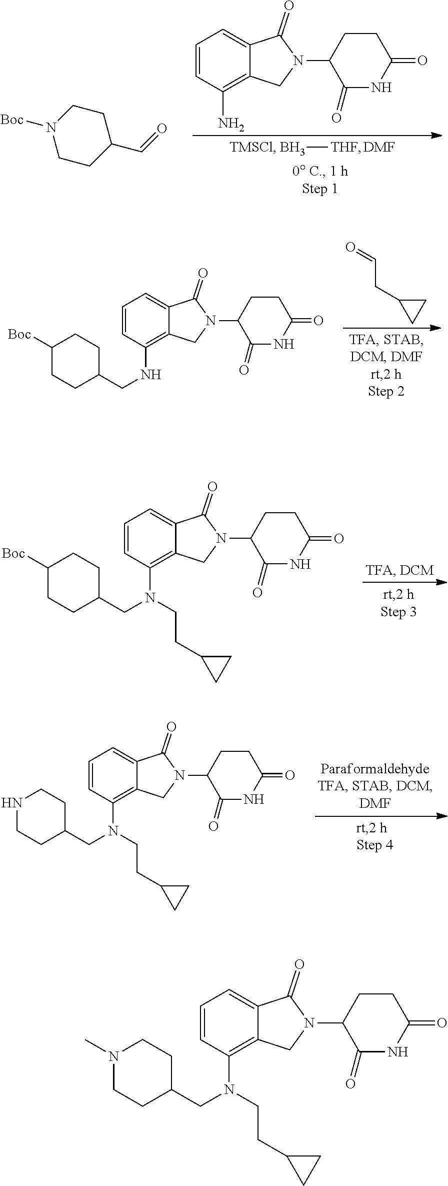

### Step-1: tert-butyl 4-(((2-(2,6-dioxopiperidin-3-yl)-1-oxoisoindolin-4-yl)amino)methyl)piperidine-1-carboxylate

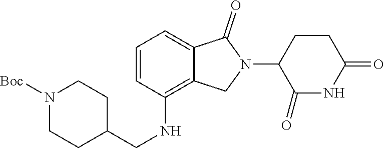 [SMILES: CC(C)(C)OC(=O)N1CCC(CNc2cccc3c2CN(C2CCC(=O)NC2=O)C3=O)CC1]

A solution of tert-butyl 4-formylpiperidine-1-carboxylate (1 g, 4.689 mmol, 1 equiv) and lenalidomide (1.46 g, 5.627 mmol, 1.2 equiv) in DMF (20 mL) was treated with TMSCl (5.09 g, 46.890 mmol, 10 equiv) for 1 h at 0° C. under nitrogen atmosphere followed by the addition of BH₃-THF (23.5 mL, 23 mmol, 5 equiv) dropwise at 0° C. The resulting mixture was stirred for 1 h at 0° C. under nitrogen atmosphere. The reaction was quenched with Water/Ice at 0° C. The resulting mixture was extracted with EtOAc (3×50 mL). The combined organic layers were washed with brine (3×100 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 40% to 70% gradient in 20 min; detector, UV 254 nm. This resulted in tert-butyl 4-(((2-(2,6-dioxopiperidin-3-yl)-1-oxoisoindolin-4-yl)amino)methyl)piperidine-1-carboxylate (1.36 g, 64%) as an off-white solid.

LCMS: 357.2 [M-Boc+H]⁺

### Step-2: tert-butyl 4-(((2-cyclopropylethyl)(2-(2,6-dioxopiperidin-3-yl)-1-oxoisoindolin-4-yl)amino)methyl)piperidine-1-carboxylate

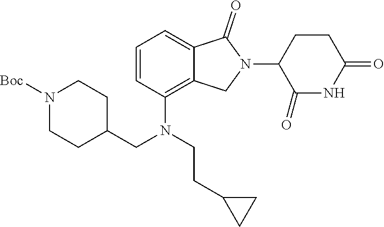 [SMILES: CC(C)(C)OC(=O)N1CCC(CN(CCC2CC2)c2cccc3c2CN(C2CCC(=O)NC2=O)C3=O)CC1]

A solution of tert-butyl 4-(((2-(2,6-dioxopiperidin-3-yl)-1-oxoisoindolin-4-yl)amino)methyl)piperidine-1-carboxylate (1.3 g, 2.847 mmol, 1 equiv) and 2-cyclopropylacetaldehyde (4.79 g, 56.940 mmol, 20 equiv) in DCM/DMF (33.25 mL/7.98 mL) was treated with TFA (974.0 mg, 8.541 mmol, 3 equiv) for overnight at room temperature under nitrogen atmosphere followed by the addition of STAB (3.02 g, 14.235 mmol, 5 equiv) in portions at 0° C. The resulting mixture was stirred for 2 h at room temperature under nitrogen atmosphere. The resulting mixture was filtered, the filter cake was washed with DCM (3×5 mL). The filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 220 nm. This resulted in tert-butyl 4-(((2-cyclopropylethyl)(2-(2,6-dioxopiperidin-3-yl)-1-oxoisoindolin-4-yl)amino)methyl)piperidine-1-carboxylate (1.3 g, 87%) as a yellow solid.

LCMS: 469.25 [M-tBu+H]⁺

### Step-3: 3-(4-((2-cyclopropylethyl)(piperidin-4-ylmethyl)amino)-1-oxoisoindolin-2-yl)piperidine-2,6-dione

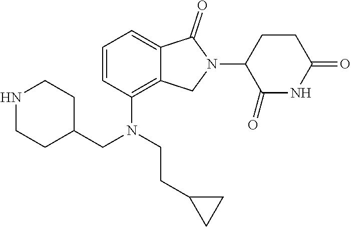 [SMILES: O=C1CCC(N2Cc3c(cccc3N(CCC3CC3)CC3CCNCC3)C2=O)C(=O)N1]

To a stirred mixture of tert-butyl 4-(((2-cyclopropylethyl)(2-(2,6-dioxopiperidin-3-yl)-1-oxoisoindolin-4-yl)amino)methyl)piperidine-1-carboxylate (1.3 g, 2.478 mmol, 1 equiv) in DCM (12.5 ml) was added TFA (2.5 mL) dropwise at 0° C. under air atmosphere. The resulting mixture was stirred for 2 h at room temperature under the air atmosphere. The resulting mixture was concentrated under vacuum. This resulted in 3-(4-((2-cyclopropylethyl)(piperidin-4-ylmethyl)amino)-1-oxoisoindolin-2-yl)piperidine-2,6-dione (2 g, crude) as a brown solid.

LCMS: 425.25 [M+H]⁺

### Step-4:3-(4-((2-cyclopropylethyl)((1-methylpiperidin-4-yl)methyl)amino)-1-oxoisoindolin-2-yl)piperidine-2,6-dione

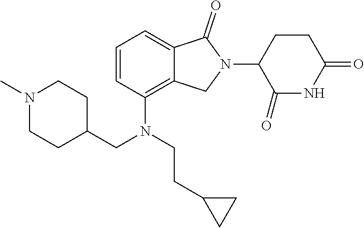 [SMILES: CN1CCC(CN(CCC2CC2)c2cccc3c2CN(C2CCC(=O)NC2=O)C3=O)CC1]

A solution of 3-(4-((2-cyclopropylethyl)(piperidin-4-ylmethyl)amino)-1-oxoisoindolin-2-yl)piperidine-2,6-dione (300 mg, 0.707 mmol, 1 equiv) and Paraformalclehyde (155.7 mg, 3.535 mmol, 5 equiv) in DCM/DMF (7.5 mL/1.8 mL) was treated with TFA (241.7 mg, 2.121 mmol, 3 equiv) for 2 h at room temperature under nitrogen atmosphere followed by the addition of STAB (748.8 mg, 3.535 mmol, 5 equiv) in portions at 0° C. The resulting mixture was filtered, the filter cake was washed with DCM (3×5 mL). The filtrate was concentrated under reduced pressure. The crude product was purified by Prep-HPLC with the following conditions (Column: XBridge Shield RP18 OBD Column 30*150 mm, 5 m; Mobile Phase A: Water (10 mmol/L NH4HCO3), Mobile Phase B: ACN; Flow rate: 60 mL/min mL/min; Gradient: 14% B to 34% B in 10 min; Wave Length: 254 nm/220 nm; RT1 (min): 11.05) to afford 3-(4-((2-cyclopropylethyl)((1-methylpiperidin-4-yl)methyl)amino)-1-oxoisoindolin-2-yl)piperidine-2,6-dione (8.3 mg, 2.64% yield) as an off-white solid.

LCMS: (ES, m/z)=439. 25 [M+H]⁺

¹H NMR (400 MHz, DMSO-d₆) δ 10.83 (s, 1H), 7.38 (d, J=7.7 Hz, 1H), 7.23-7.14 (m, 1H), 7.14-7.05 (m, 1H), 5.11 (dd, J=13.2, 5.1 Hz, 1H), 4.42-4.28 (m, 2H), 3.30-3.23 (m, 2H), 3.12-3.06 (m, 2H), 2.90 (ddd, J=17.1, 13.6, 5.4 Hz, 1H), 2.69 (d, J=10.9 Hz, 2H), 2.59 (ddd, J=17.2, 4.4, 2.2 Hz, 1H), 2.48-2.43 (m, 1H), 2.09 (s, 2H), 2.01 (ddq, J=10.5, 5.2, 2.7, 2.2 Hz, 1H), 1.71 (t, J=11.4 Hz, 2H), 1.61 (d, J=12.6 Hz, 2H), 1.40 (dt, J=10.9, 3.7 Hz, 1H), 1.32 (qd, J=7.1, 3.2 Hz, 2H), 1.23-1.06 (m, 2H), 0.68-0.56 (m, 1H), 0.40-0.28 (m, 2H).

Method: Mobile Phase A: Water/0.1% FA, Mobile Phase B: Acetonitrile/0.1% FA. Flow rate:1.5000 mL/min. COLUMN: HALO-C18 (30×3.0) mm, 2μ, Rt (min): 0.757; Area %—99.881.

### Compound 715

### 3-(4-((2-cyclopropylethyl)((1-(3,3,3-trifluoropropyl)piperidin-4-yl)methyl)amino)-1-oxoisoindolin-2-yl)piperidine-2,6-dione

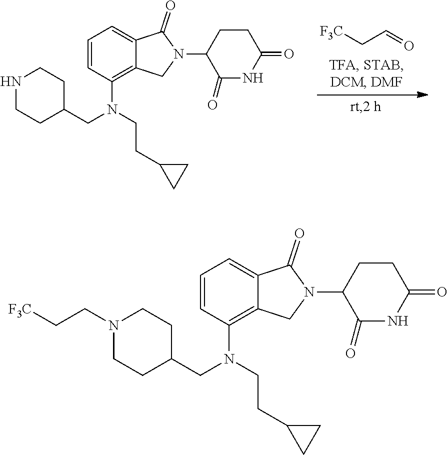 [SMILES: O=C1CCC(N2Cc3c(cccc3N(CCC3CC3)CC3CCNCC3)C2=O)C(=O)N1 ; O=CCC(F)(F)F ; O=C1CCC(N2Cc3c(cccc3N(CCC3CC3)CC3CCN(CCC(F)(F)F)CC3)C2=O)C(=O)N1]

A solution of 3-{4-[(2-cyclopropylethyl)(piperidin-4-ylmethyl)amino]-1-oxo-3H-isoindol-2-yl}piperidine-2,6-dione (300 mg, 0.707 mmol, 1 equiv) and 3,3,3-trifluoropropanal (95.0 mg, 0.848 mmol, 1.2 equiv) in DCM/DMF (7.5 mL/1.8 mL) was treated with TFA (241.7 mg, 2.121 mmol, 3 equiv) for 2 h at room temperature under nitrogen atmosphere followed by the addition of STAB (748.8 mg, 3.535 mmol, 5 equiv) in portions at 0° C. The resulting mixture was stirred for 2 h at room temperature under nitrogen atmosphere. The resulting mixture was filtered, the filter cake was washed with DCM (2×5 mL). The filtrate was concentrated under reduced pressure. The crude product was purified by Prep-HPLC with the following conditions (Column: Sunfire prep C18 column, 30*150 mm, 5 m; Mobile Phase A: Water (0.1% FA), Mobile Phase B: ACN; Flow rate: 60 mL/min mL/min; Gradient: 2% B to 22% B in 10 min; Wave Length: 254 nm/220 nm; RT1 (min): 12.13) to afford 3-(4-((2-cyclopropylethyl)((1-(3,3,3-trifluoropropyl)piperidin-4-yl)methyl)amino)-1-oxoisoindolin-2-yl)piperidine-2,6-dione (51.6 mg, 14%) as an off-white solid.

LCMS: (ES, m/z)=521.30 [M+H]⁺

¹H NMR (400 MHz, DMSO-d₆) δ 10.98 (s, 1H), 7.37 (t, J=7.7 Hz, 1H), 7.18 (d, J=7.3 Hz, 1H), 7.10 (d, J=8.1 Hz, 1H), 5.11 (dd, J=13.2, 5.1 Hz, 1H), 4.46-4.25 (m, 2H), 3.09 (dt, J=10.5, 5.2 Hz, 2H), 2.98-2.78 (m, 3H), 2.60 (ddd, J=17.1, 4.5, 2.2 Hz, 1H), 2.48-2.33 (m, 4H), 2.01 (dtd, J=12.7, 5.2, 2.3 Hz, 1H), 1.83 (t, J=11.4 Hz, 2H), 1.63 (d, J=12.6 Hz, 2H), 1.47 (dt, J=15.3, 3.9 Hz, 1H), 1.32 (qd, J=7.1, 3.0 Hz, 2H), 1.13 (qd, J=12.2, 3.8 Hz, 2H), 0.63 (ddt, J=10.1, 7.5, 3.6 Hz, 1H), 0.40-0.30 (m, 2H)

Method: Mobile Phase A: Water/5 mM NH₄HCO₃, Mobile Phase B: Acetonitrile. Flow rate:1.5000 mL/min. COLUMN: HPH-C18 (50×3.0) mm, 2.7μ, Rt (min): 1.446; Area %—99.762.

### Trifluoroacetate Salt Forms of Compound 716 and 716A

### 3-(4-((2-cyclopropylethyl)((1r,4r)-4-(4,4-difluoropiperidin-1-yl)cyclohexyl)amino)-1-oxoisoindolin-2-yl)piperidine-2,6-dione 2,2,2-trifluoroacetate (716) and 3-(4-((2-cyclopropylethyl)((1s,4s)-4-(4,4-difluoropiperidin-1-yl)cyclohexyl)amino)-1-oxoisoindolin-2-yl)piperidine-2,6-dione tris(2,2,2-trifluoroacetate) (716A)

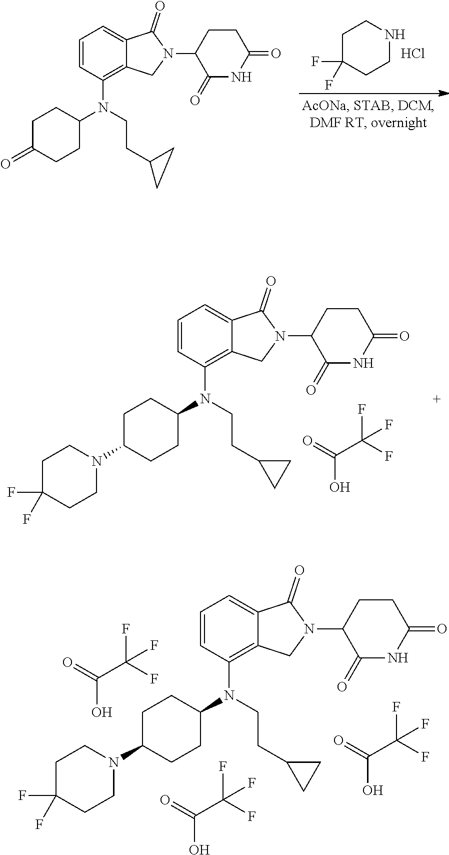

A solution of 3-{4-[(2-cyclopropylethyl)(4-oxocyclohexyl)amino]-1-oxo-3H-isoindol-2-yl}piperidine-2,6-dione (150 mg, 0.354 mmol, 1 equiv), 4,4-difluoropiperidine (214.5 mg, 1.770 mmol, 5 equiv) in DCM (4 mL) and DMF (1 mL) was treated with AcONa (87.1 mg, 1.062 mmol, 3 equiv) for overnight at room temperature under nitrogen atmosphere followed by the addition of STAB (375.3 mg, 1.770 mmol, 5 equiv) dropwise at 0° C. The resulting mixture was stirred for an additional 2 h at room temperature. The resulting mixture was diluted with water and extracted with EtOAc (3×20 mL). The combined organic layers were washed with brine (2×50 mL), dried over anhydrous Na₂SO₄. The crude product (100 mg) was purified by Prep-HPLC with the following conditions (Column: Xselect CSH F-Phenyl OBD column 30*250 mm, 5 μm; Mobile Phase A: Water (0.05% TFA), Mobile Phase B: ACN; Flow rate: 60 mL/min mL/min; Gradient: 2% B to 16% B in 10 min; Wave Length: 254 nm/220 nm; RT1 (min): 12.08/18.55) to afford 3-(4-((2-cyclopropylethyl)((1r,4r)-4-(4,4-difluoropiperidin-1-yl)cyclohexyl)amino)-1-oxoisoindolin-2-yl)piperidine-2,6-dione 2,2,2-trifluoroacetate (23.5 mg, 13% yield) as an off-white solid and 3-(4-((2-cyclopropylethyl)((1s,4s)-4-(4,4-difluoropiperidin-1-yl)cyclohexyl)amino)-1-oxoisoindolin-2-yl)piperidine-2,6-dione (56.6 mg, crude) as an off-white solid. The crude product (56.6 mg) was purified again via Prep-HPLC with the following conditions (Column: Xselect CSH F-Phenyl OBD column 30*250 mm, 5 μm; Mobile Phase A: Water (0.05% TFA), Mobile Phase B: ACN; Flow rate: 60 mL/min; Gradient: 2% B to 16% B in 15 min; Wave Length: 254 nm/220 nm; RT1 (min): 12.08/18.55) to 3-(4-((2-cyclopropylethyl)((1s,4s)-4-(4,4-difluoropiperidin-1-yl)cyclohexyl)amino)-1-oxoisoindolin-2-yl)piperidine-2,6-dione tris(2,2,2-trifluoroacetate) (24.6 mg, 13% yield) as an off-white solid.

### 3-(4-((2-cyclopropylethyl)((1r,4r)-4-(4,4-difluoropiperidin-1-yl)cyclohexyl)amino)-1-oxoisoindolin-2-yl)piperidine-2,6-dione 2,2,2-trifluoroacetate

LCMS: 529.30 (M+H). Method: Mobile Phase A: Water/0.1% FA. Mobile Phase B: Acetonitrile/0.07% FA. Flow rate:1.5 mL/min. COLUMN: HALO-C18 (30×3.0) mm, 2.0μ. Rt (min): 0.840; Area %—99.740.

¹H NMR (400 MHz, DMSO-d₆): δ 10.97 (s, 1H), 9.73 (s, 1H), 7.43 (t, J=7.7 Hz, 1H), 7.31 (dd, J=13.7, 7.7 Hz, 2H), 5.11 (dd, J=13.2, 5.1 Hz, 1H), 4.42-4.21 (m, 3H), 3.52 (s, 2H), 3.34 (s, 1H), 3.18 (p, J=7.0, 6.6 Hz, 5H), 2.92 (ddd, J=18.0, 13.4, 5.4 Hz, 1H), 2.60 (d, J=17.5 Hz, 1H), 2.49-2.22 (m, 5H), 2.03 (d, J=31.9 Hz, 3H), 1.89 (d, J=14.9 Hz, 2H), 1.59 (h, J=11.3, 10.2 Hz, 4H), 1.14 (q, J=7.0 Hz, 2H), 0.63 (td, J=7.3, 3.9 Hz, 1H), 0.38-0.29 (m, 2H), −0.03-−0.10 (m, 2H).

¹⁹F NMR (400 MHz, DMSO-d₆): δ −74.233 (s, 3), −94.383˜−95.013 (m, 1), −101.079˜−101.711 (m, 1).

### 3-(4-((2-cyclopropylethyl)((1s,4s)-4-(4,4-difluoropiperidin-1-yl)cyclohexyl)amino)-1-oxoisoindolin-2-yl)piperidine-2,6-dione tris(2,2,2-trifluoroacetate)

LCMS: 529.30 (M+H). Method: Mobile Phase A: Water/0.1% FA. Mobile Phase B: Acetonitrile/0.07% FA. Flow rate:1.5 mL/min. COLUMN: HALO-C18 (30×3.0) mm, 2.0μ. Rt (min):0.882; Area %—99.896.

¹H NMR (400 MHz, DMSO-d₆): δ 11.02 (s, 1H), 9.52 (s, 1H), 7.51-7.35 (m, 3H), 5.16 (dd, J=13.2, 5.2 Hz, 1H), 4.41 (d, J=17.6 Hz, 1H), 4.29 (d, J=17.5 Hz, 1H), 3.55 (d, J=22.5 Hz, 3H), 3.40 (s, 1H), 3.23-3.08 (m, 4H), 2.91 (d, J=18.1 Hz, 1H), 2.73 (s, 1H), 2.63 (s, 1H), 2.45-2.37 (m, 3H), 2.26 (d, J=15.5 Hz, 2H), 2.10-1.94 (m, 2H), 1.83 (d, J=12.9 Hz, 2H), 1.68 (d, J=28.8 Hz, 3H), 1.50 (t, J=12.5 Hz, 2H), 1.22 (q, J=7.5, 6.8 Hz, 3H), 0.55 (d, J=5.2 Hz, 1H), 0.37-0.28 (m, 2H), −0.08 (td, J=5.3, 3.8 Hz, 2H).

¹⁹F NMR (400 MHz, DMSO-d₆): δ −74.752 (s, 10), −94.673˜−95.303 (m, 1), −101.012˜−101.637 (m, 1).

### II. CK1α Degrading Effects of the Compounds

a. AML Cell Viability

### Cell Culture

MV4-11 cells were obtained from ATCC and grown in IMDM supplemented with 10% FBS. The cells were maintained at 37° C. under 5% CO₂.

### Cell Viability Assay

The efficacy of the present disclosure to reduce viability of acute myeloid leukemia (AML) cells was assessed using RealTime-Glo Cell Viability Assay (Promega) according to the manufacturer's protocol. Briefly, Compounds of the present disclosure or reference compounds (lenalidomide, cytarabine, or azacytidine) was added to 1,500 MV-4-11 cells in a white 384-well plate (8-point dose response, top concentration 31.6 M, half-log serial dilution, final DMSO concentration 0.1%) along with the RealTime Glo reagent (final concentration of 1×). The luminescence was read twice daily for 3 days. The luminescence was normalized to that of DMSO-treated samples. IC₅₀ values were obtained using three-parameter non-linear regressions using Prism (GraphPad).

The efficacy of additional compounds of the present disclosure in reducing AML cell viability was determined by treating MV-4-11 cells at various concentrations (final DMSO concentration of 0.10%). Cell viability was measured using the RealTime-Glo reagent after 44 hrs of treatment. The results are summarized in TABLEs below. The compounds are classified based on IC₅₀ values as follows.
A: IC₅₀ is less than or equal to 1×10⁻⁷;
B: IC₅₀ is greater than 1×10⁻⁷ & less than or equal to 1×10⁻⁶;
C: IC₅₀ is greater than 1×10⁻⁶;
NC: not calculated (no activity detected, <25% viability reduction at 1 uM)
   b. CK1α Degradation

### Cell Culture

MM.1S and NCCIT cells were obtained from ATCC and grown in RPMI-1640 supplemented with 10% FBS. MV-4-11 cells were obtained from ATCC and grown in IMDM supplemented with 10% FBS. The cells were maintained at 37° C. under 5% CO₂.

### Jess Assay

Compounds of the present disclosure was added to cells (final DMSO concentration of 0.1%) and incubated for 16 hours. CK1α levels were determined in MV-4-11 cells using Jess (Protein Simple). Briefly, whole cell lysates were prepared in lysis buffer (1×RIPA buffer [Thermo, 89900], 2 mM MgCl₂ [Sigma, M1028], 0.05% Benzonase [Sigma, E1014], 1× Pierce protease inhibitor tablets EDTA-free [Thermo, A32965]). 6-20 μg of the lysates were analyzed using Jess with antibodies against CK1α (Abcam, ab108296) and β-actin (R&D Systems, MAB8929). Protein levels were normalized to β-actin levels. DC₅₀ values were obtained using four-parameter non-linear regressions using Prism (GraphPad).

The efficacy of CK1α degradation of a part of compounds of the present disclosure was determined by treating MV-4-11 cells at 1 M (final DMSO concentration of 0.1%) for 16 hours. CK1α levels were determined using Jess and normalized to β-actin levels. The results are summarized in TABLE 1. The compounds are classified based on amount of CK1α degradation (% reduction of CK1α compared to DMSO) as follows.
A: Degradation is greater than or equal to 75%;
B: Degradation is less than 7500 & greater than or equal to 50%;
C: Degradation is less than 5000;
ND: not determined.

TABLE 1

| | CK1α | |
|---|---|---|
| | degradation | Cell viability |
| Compound No. | classification | classification |
| 4 | A | A |
| 46 | A | A |
| 47 | A | A |
| 48 | A | A |
| 49 | C | NC |
| 50 | A | A |
| 51 | B | B |
| 52 | A | B |
| 53 | C | B |
| 55 | A | B |
| 56 | B | B |
| 57 | A | A |
| 60 | A | A |
| 63 | A | A |
| 66 | B | B |
| 67 | A | A |
| 68 | A | A |
| 69 | B | B |
| 80 | A | A |
| 82 | B | A |
| 87 | A | A |
| 88 | A | A |
| 89 | A | A |
| 90 | A | A |
| 115 | B | NC |
| 116 | A | A |
| 247 | ND | B |
| 251 | A | A |
| 323 | A | A |
| 324 | A | A |
| 327 | ND | A |
| 328 | ND | A |
| 332 | ND | A |
| 333 | A | A |
| 334 | ND | NC |
| 340 | ND | B |
| 341 | ND | B |
| 344 | ND | B |
| 345 | ND | B |
| 346 | ND | B |
| 347 | ND | B |
| 349 | ND | C |
| 352 | ND | A |
| 353 | ND | B |
| 354 | ND | NC |
| 355 | ND | A |
| 356 | ND | A |
| 358 | A | A |
| 359 | ND | A |
| 360 | ND | A |
| 361 | ND | B |
| 362 | ND | B |

### Flow Cytometry

Compounds of the present disclosure were added to cells (final DMSO concentration of 0.5%) and incubated for 16 hours. CK1α level was determined in MV4-11 cells using flow cytometry (Luminex, Guava EasyCyte Plus). Briefly, cells were prepared with sequential 10% formalin and 70% methanol fixation. Staining was performed with an CK1α antibody (AbCam, ab206652) and PE-labelled goat anti-rabbit IgG (Invitrogen, P-2771MP). GuavaSoft Incyte software was used to analyse the flow cytometry data. Protein levels relative to DMSO are calculated for CK1α.

The efficacy of CK1α degradation of other part of compounds of the present disclosure was determined by treating MV4-11 cells at 1 M (Final DMSO concentration of 0.5%) for 16 hours. Protein levels relative to DMSO are calculated for CK1α. The results are summarized in TABLE 2. The compounds are classified based on the amount of CK1α degradation (% reduction of CK1α compared to DMSO) as follows.
A: Degradation is greater than or equal to 75%;
B: Degradation is less than 75% & greater than or equal to 50%;
C: Degradation is less than 50%;
ND: not determined.

TABLE 2

| | CK1α | |
|---|---|---|
| | degradation | Cell viability |
| Compound No. | classification | classification |
| 91 | B | A |
| 92 | B | A |
| 96 | B | B |
| 97 | C | NC |
| 102 | B | B |
| 103 | A | B |
| 104 | A | B |
| 105 | A | B |
| 106 | B | B |
| 107 | A | B |
| 108 | A | A |
| 109 | A | B |
| 123 | A | A |
| 124 | A | A |
| 125 | A | B |
| 126 | A | A |
| 143 | A | B |
| 148 | A | B |
| 149 | B | C |
| 150 | B | C |
| 151 | B | C |
| 181 | B | B |

The efficacy of CK1α degradation of other part of compounds of the present disclosure was determined by treating MV4-11 cells at 316 nM (Final DMSO concentration of 0.5%) for 16 hours. Protein levels relative to DMSO are calculated for CK1α. The results are summarized in TABLE 3. The compounds are classified based on the amount of CK1α degradation (% reduction of CK1α compared to DMSO) as follows.
A: Degradation is greater than or equal to 75%;
B: Degradation is less than 75% & greater than or equal to 50%;
C: Degradation is less than 50%;
ND: not determined.

TABLE 3

| | CK1α | |
|---|---|---|
| | degradation | Cell viability |
| Compound No. | classification | classification |
| 93 | A | A |
| 94 | A | A |
| 95 | C | B |
| 166 | A | B |
| 167 | A | B |
| 168 | A | B |
| 169 | A | A |
| 223 | A | A |
| 235 | A | A |
| 236 | A | B |
| 237 | A | A |
| 238 | A | A |
| 239 | C | NC |
| 240 | C | B |
| 241 | C | NC |
| 242 | A | A |
| 243 | C | NC |
| 244 | C | NC |
| 245 | C | NC |
| 246 | C | NC |
| 255 | B | B |
| 267 | A | A |
| 268 | A | A |
| 273 | B | NC |
| 305 | A | A |
| 306 | A | B |
| 307 | A | A |
| 308 | A | A |
| 309 | A | A |
| 310 | B | A |
| 311 | A | A |
| 312 | A | A |
| 313 | A | A |
| 325 | A | A |
| 325A | A | A |
| 326 | A | A |
| 326A | A | A |
| 329 | B | B |
| 330 | A | A |
| 331 | ND | NC |
| 512 | B | B |
| 513 | A | B |
| 514 | B | B |
| 515 | B | NC |
| 516 | A | A |
| 517 | A | A |
| 518 | A | B |
| 519 | B | B |
| 521 | A | A |
| 522 | A | A |
| 523 | B | B |
| 530 | C | C |
| 532 | A | A |
| 533 | A | A |
| 534 | A | A |
| 535 | B | A |
| 536 | C | B |
| 515A | B | B |
| 539 | B | B |
| 539A | A | A |
| 540 | B | B |
| 541 | A | A |
| 541A | B | B |
| 542 | A | A |
| 543 | A | A |
| 546 | A | A |
| 547 | A | A |
| 548 | C | B |
| 548A | B | B |

### Western Blot

Compounds of the present disclosure was added to cells (final DMSO concentration of 0.1%) and incubated for 4 hours. CK1α levels were determined in MV-4-11 cells using Western blot analysis. Whole cell lysates were prepared in lysis buffer (1×RIPA buffer [Thermo, 89900], 2 mM MgCl₂ [Sigma, M1028], 0.05% Benzonase [Sigma, E1014], 1× Pierce protease inhibitor tablets EDTA-free [Thermo, A32965]). 6-20 g of the lysates were analyzed using Western blot with antibodies against CK1α (Abcam, ab108296) and β-actin (R&D Systems, MAB8929).

The efficacy of CK1α degradation of other part of compounds of the present disclosure was determined by treating MV4-11 cells at 1 μM (Final DMSO concentration of 0.1%) for 4 hours. Protein levels relative to DMSO are calculated for CK1α. The results are summarized in TABLE 4. The compounds are classified based on the amount of CK1α degradation (% reduction of CK1α compared to DMSO) as follows.
A: Degradation is greater than or equal to 75%;
B: Degradation is less than 75% & greater than or equal to 50%;
C: Degradation is less than 500%;
ND: not determined.

TABLE 4

| | CK1α | |
|---|---|---|
| | degradation | Cell viability |
| Compound No. | classification | classification |
| 550 | ND | B |
| 554 | ND | B |
| 554A | ND | B |
| 557 | ND | B |
| 558-2 | ND | A |
| 561 | ND | A |
| 562 | ND | B |
| 573 | ND | A |
| 574 | ND | B |
| 575 | ND | B |
| 576 | ND | B |
| 580 | ND | C |
| 582 | ND | A |
| 583 | ND | B |
| 603 | A | A |
| 604 | A | A |
| 611 | ND | A |
| 612 | A | A |
| 612A | ND | B |
| 613 | A | A |
| 613A | ND | B |
| 614 | ND | B |
| 614A | ND | NC |
| 615 | A | A |
| 616 | A | A |
| 616A | ND | B |
| 619 | ND | B |
| 627 | ND | C |
| 627A | ND | C |
| 628 | ND | B |
| 628A | ND | C |
| 634 | B | B |
| 635 | A | A |
| 636 | A | B |
| 636A | ND | C |

### CK1α HiBiT Assay

CK1a_HiBiT_Jurkat cells were culture in RPMI 1640 medium (Gibco, 11875093) with 10% FBS under standard tissue culture conditions. Briefly, Compounds of the present disclosure was added to 30,000 cells in a white 96-well plate (9-point dose response, top concentration 31.6 M, half-log serial dilution, final DMSO concentration 0.5%) and incubated for 16 hours. CK1a level was determined in along with the Nano-Glo HiBiT Lytic Detection system [Promega N3050] with final concentration of 1 x. The luminescence was normalized to that of DMSO-treated samples. DC₅₀ values were obtained using three-parameter non-linear regressions using Prism (GraphPad). The results are summarized in TABLE 5. The compounds are classified based on DC₅₀ values as follows.
A: DC₅₀ is less than or equal to 1×10⁻⁷;
B: DC₅₀ is greater than 1×10⁻⁷ & less than or equal to 1×10⁻⁶;
C: DC₅₀ is greater than 1×10⁻⁶;
NA: not calculated (no activity detected, <25% viability reduction at 1 uM)

TABLE 5

| | CK1α | |
|---|---|---|
| | degradation | Cell viability |
| Compound No. | classification | classification |
| 641 | ND | C |
| 642 | ND | A |
| 643 | ND | A |
| 644 | ND | C |
| 645 | ND | A |
| 646 | ND | B |
| 647 | ND | A |
| 648 | ND | A |
| 648A | ND | NA |
| 649 | ND | B |
| 649A | ND | NA |
| 653 | ND | B |
| 653A | ND | NA |
| 654 | ND | B |
| 654A | ND | NA |
| 642A | A | C |
| 650 | A | B |
| 650A | NA | NA |
| 651 | A | C |
| 651A | C | C |
| 652 | A | C |
| 652A | C | C |
| 655 | B | C |
| 655A | NA | NA |
| 656 | B | B |
| 656A | NA | NA |
| 657 | B | C |
| 657A | NA | NA |
| 643 | A | A |
| 658 | A | B |
| 658A | B | C |
| 659 | A | B |
| 668 | A | A |
| 662 | A | B |
| 663 | A | B |
| 664 | A | B |
| 665 | A | B |
| 646 | A | B |
| 666 | A | A |
| 669 | A | B |
| 670 | A | B |
| 669A | B | NA |
| 670A | B | NA |
| 671 | A | B |
| 671A | B | NA |
| 672 | A | B |
| 672A | B | NA |
| 673 | A | C |
| 673A | B | NA |
| 674 | A | A |
| 674A | B | NA |
| 675 | A | B |
| 675A | B | NA |
| 660 | A | B |
| 680 | A | B |
| 680A | B | NA |
| 681 | A | A |
| 681A | B | NA |
| 682 | A | A |
| 682A | B | NA |
| 703 | A | B |
| 678 | B | B |
| 679 | ND | B |
| 683 | A | A |
| 683A | ND | B |
| 708 | ND | B |
| 708A | ND | A |
| 711 | ND | B |
| 711A | ND | C |
| 712 | ND | B |
| 712A | ND | C |
| 713 | ND | B |
| 713A | ND | C |
| 714 | ND | C |
| 715 | ND | NA |
| 716 | A | B |
| 716A | ND | C |

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

1. A compound of Formula (I), 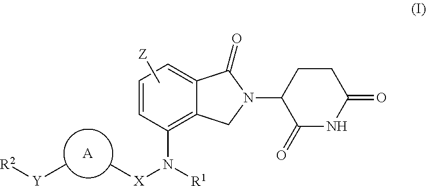
wherein
R¹ is C₁-C₈ alkyl, C₃-C₈ alkenyl, C₃-C₈ alkynyl, C₃-C₈ cycloalkyl or C₅-C₁₀ bicycloalkyl each optionally substituted with one or more halogen, OH, O—C₁-C₃ alkyl, NH₂, C₁-C₃ haloalkyl, optionally substituted C₆-C₁₀ aryl, C₁-C₃ alkyl, optionally substituted C₃-C₈ cycloalkyl, optionally substituted C₅-C₁₀ bicycloalkyl or optionally substituted C₃-C₆ heterocycloalkyl;
X is a bond or —(CH₂)ₙ— optionally substituted with one or more halogen, OH, O—C₁-C₃ alkyl, C₁-C₃ haloalkyl, C₃-C₆ cycloalkyl or C₁-C₆ alkyl, wherein the C₁-C₆ alkyl group may be taken together with the atom to which it is attached to form a C₃-C₆ spiro alkyl ring, and wherein the —(CH₂)ₙ— group may contain 0-1 double bond or triple bond;
n is an integer from 1 to 6;
A is a bond, C₄-C₈ cycloalkyl, C₅-C₁₀ bicycloalkyl, C₅-C₁₀ heterobicycloalkyl or C₃-C₈ heterocycloalkyl each optionally substituted with one of more halogen, OH, O—C₁-C₃ alkyl, CN, C₁-C₃ haloalkyl, C₁-C₃ alkyl or C₃-C₅ cycloalkyl;
Y is a bond or a —(CH₂)ₘ—;
m is an integer of 1 or 2;
Z is H or halogen;
R² is —NR¹²R⁵, —NHC(O)R⁶, —NHC(O)NR⁸(R⁹), —C(O)NHR⁵, five or six-membered heteroaryl, or C₃-C₈ heterocycloalkyl or C₅-C₁₀ heterobicycloalkyl ring optionally substituted with one of more halogen, OH, O—C₁-C₃ alkyl, CN, C₁-C₃ haloalkyl, C₁-C₃ alkyl or C₃-C₅ cycloalkyl;
R⁵ is H, C₁-C₅ alkyl, C₃-C₇ cycloalkyl, five or six-membered heteroaryl, C₅-C₁₀ bicycloalkyl, (C₃-C₆ cycloalkyl)-C₁-C₃ alkyl, (C₃-C₆ heterocycloalkyl)-C₁-C₃ alkyl, (C₆-C₁₀ aryl)-C₁-C₃ alkyl, (C₁-C₅-heteroaryl)-C₁-C₃ alkyl or C₃-C₇ heterocycloalkyl each optionally substituted with one or more halogen, CN, OH, O—(C₁-C₃ haloalkyl), O—(C₁-C₃ alkyl), C₁-C₃ haloalkyl, CH₃SO₂—, optionally substituted C₃-C₆ cycloalkyl, optionally substituted C₃-C₇ heterocycloalkyl or C(O)NR⁸(R⁹);
R⁶ is C₁-C₆ alkyl, C₃-C₆ cycloalkyl or C₃-C₆ heterocycloalkyl each optionally substituted with one or more halogen, C₁-C₃ haloalkyl, optionally substituted C₃-C₆ cycloalkyl, or NR⁸(R⁹);
R⁸ and R⁹ are each independently H, substituted C₁-C₃ alkyl or taken together with the nitrogen to which they are attached to form a 4-6 membered heterocyclic ring optionally substituted with one or more halogen or C₁-C₃ haloalkyl;
R¹² is H, C₁-C₅ alkyl, or taken together with R⁵ and the nitrogen to which it is attached to form (i) C₃-C₈ cycloalkyl or heterocycloalkyl ring or (ii) C₅-C₁₀ bicycloalkyl or heterobicycloalkyl ring, each optionally substituted with one or more halogen, C₁-C₃ haloalkyl, OH, O—(C₁-C₃ alkyl), O—(C₁-C₃ haloalkyl) or CN.
each stereocenter in the compound of Formula (I) is independently the R-enantiomer, the S-enantiomer or a mixture of R- and S-enantiomers; and
each double bond in the compound of Formula (I) is independently cis or trans,
or a pharmaceutically acceptable salt, enantiomer, stereoisomer or prodrug thereof.

2. The compound of claim 1, wherein the compound is represented by Formula (I-a) and
X is a bond or —(CH₂)ₙ—, 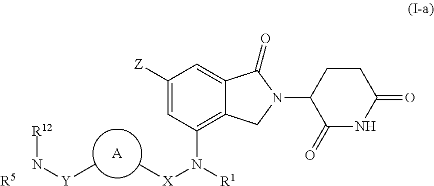
or a pharmaceutically acceptable salt, enantiomer, stereoisomer or prodrug thereof.

3. The compound of claim 2, wherein
R¹ is C₁-C₈ alkyl or C₃-C₈ cycloalkyl each optionally substituted with one or more halogen, OH, NH₂, C₁-C₃ alkyl, C₁-C₃ haloalkyl, O—(C₁-C₃ alkyl), optionally substituted C₆ aryl, optionally substituted C₃-C₈ cycloalkyl, optionally substituted C₃-C₆ heterocycloalkyl or optionally substituted C₆-C₈ bicycloalkyl,
or a pharmaceutically acceptable salt, enantiomer, stereoisomer or prodrug thereof.

4. The compound of claim 2, wherein
A is C₄-C₆ cycloalkyl, C₄-C₆ heterocycloalkyl or C₅-C₇ bicycloalkyl each optionally substituted with one or more halogen, OH, C₁-C₃ alkyl or C₁-C₃ haloalkyl,
or a pharmaceutically acceptable salt, enantiomer, stereoisomer or prodrug thereof.

5. The compound of claim 2, wherein
Y is a bond or —CH₂—,
or a pharmaceutically acceptable salt, enantiomer, stereoisomer or prodrug thereof.

6. The compound of claim 2, wherein
R⁵ is H, C₁-C₅ alkyl, C₃-C₆ cycloalkyl, 6-membered heteroaryl, C₅ bicycloalkyl, (C₃-C₄ cycloalkyl)-C₁-C₂ alkyl, (C₃-C₄ heterocycloalkyl)-C₁-C₂ alkyl, (C₆ aryl)-C₁-C₂ alkyl, (C₅ heteroaryl)-C₁-C₂ alkyl or C₃-C₄ heterocycloalkyl each optionally substituted with one or more halogen, CN, OH, O—(C₁-C₃ haloalkyl), O—(C₁-C₃ alkyl), C₁-C₃ haloalkyl, CH₃SO₂— or C(O)NR⁸(R⁹);
or a pharmaceutically acceptable salt, enantiomer, stereoisomer or prodrug thereof.

7. The compound of claim 2, wherein
R¹² is H, C₁-C₅ alkyl, or taken together with R⁵ and the nitrogen to which it is attached to form a C₃-C₈ heterocycloalkyl ring optionally substituted with one or more halogen or C₁-C₃ haloalkyl.
or a pharmaceutically acceptable salt, enantiomer, stereoisomer or prodrug thereof.

8. The compound of claim 1, wherein the compound is represented by Formula (I-b) and
X is a bond or —(CH₂)ₙ—, 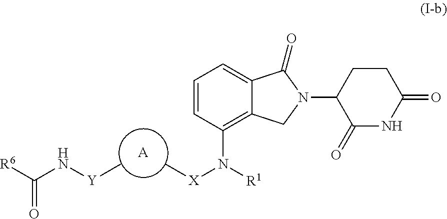
or a pharmaceutically acceptable salt, enantiomer, stereoisomer or prodrug thereof.

9. The compound of claim 8, wherein
R¹ is C₃-C₆ alkyl optionally substituted with one or more halogen, OH, O—C₁-C₃ alkyl, NH₂, C₁-C₃ haloalkyl,
or a pharmaceutically acceptable salt, enantiomer, stereoisomer or prodrug thereof.

10. The compound of claim 8, wherein
A is bond or C₄-C₆ cycloalkyl optionally substituted with one or more halogen, OH, O—C₁-C₃ alkyl, NH₂, C₁-C₃ haloalkyl,
or a pharmaceutically acceptable salt, enantiomer, stereoisomer or prodrug thereof.

11. The compound of claim 8, wherein
Y is a bond,
or a pharmaceutically acceptable salt, enantiomer, stereoisomer or prodrug thereof.

12. The compound of claim 8, wherein
R⁶ is C₁-C₆ alkyl, optionally substituted with NR⁸(R⁹),
or a pharmaceutically acceptable salt, enantiomer, stereoisomer or prodrug thereof.

13. The compound of claim 1, wherein the compound is represented by Formula (I-c) and
X is a bond or —(CH₂)ₙ—, 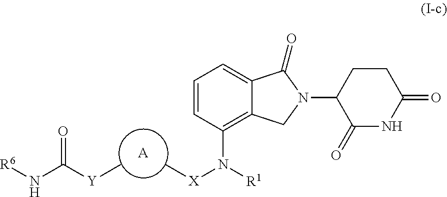
or a pharmaceutically acceptable salt, enantiomer, stereoisomer or prodrug thereof.

14. The compound of claim 13, wherein
R¹ is C₁-C₆ alkyl optionally substituted with optionally substituted C₃-C₈ cycloalkyl or optionally substituted C₃-C₆ heterocycloalkyl,
or a pharmaceutically acceptable salt, enantiomer, stereoisomer or prodrug thereof.

15. The compound of claim 13, wherein
A is bond, C₄-C₆ cycloalkyl, C₄-C₆ heterocycloalkyl or C₅-C₇ bicycloalkyl each optionally substituted with one or more halogen, OH, O—C₁-C₃ alkyl, NH₂, C₁-C₃ haloalkyl,
or a pharmaceutically acceptable salt, enantiomer, stereoisomer or prodrug thereof.

16. The compound of claim 13, wherein
Y is a bond,
or a pharmaceutically acceptable salt, enantiomer, stereoisomer or prodrug thereof.

17. The compound of claim 13, wherein
R⁵ is H or C₁-C₃ alkyl,
or a pharmaceutically acceptable salt, enantiomer, stereoisomer or prodrug thereof.

18. The compound of claim 1, wherein
each X and Y is a bond;
A is C₄-C₆ cycloalkyl;
R¹ is C₁-C₃ alkyl optionally substituted with optionally substituted C₃-C₆ cycloalkyl; and
R² is 5-membered heteroaryl,
or a pharmaceutically acceptable salt, enantiomer, stereoisomer or prodrug thereof.

19. A compound of Formula (I-d), 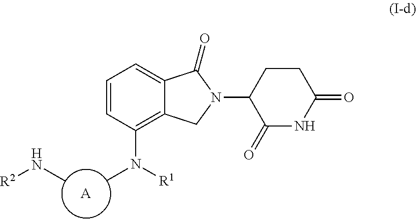
wherein
R¹ is C₁-C₃ alkyl optionally substituted with optionally substituted C₃-C₆ cycloalkyl;
A is optionally substituted C₄-C₆ cycloalkyl;
R² is C₁-C₄ alkyl or (C₃-C₄ cycloalkyl)-C₁-C₂ alkyl, each optionally substituted with one or more halogen, CN, OH, O—(C₁-C₃ haloalkyl), O—(C₁-C₃ alkyl), C₁-C₃ haloalkyl or CH₃SO₂—;
each stereocenter in the compound of Formula (I-d) is independently the R-enantiomer, the S-enantiomer or a mixture of R- and S-enantiomers; and
or a pharmaceutically acceptable salt, enantiomer, stereoisomer or prodrug thereof.

20. The compound of claim 19, wherein
R² is C₁-C₃ alkyl optionally substituted with one or more halogen, O—(C₁-C₃ haloalkyl), O—(C₁-C₃ alkyl) or C₁-C₃ haloalkyl;
or a pharmaceutically acceptable salt, enantiomer, stereoisomer or prodrug thereof.

21. (canceled)

22. (canceled)

23. (canceled)

24. A pharmaceutical composition comprising:
the compound of claim 1, or the pharmaceutically acceptable salt, enantiomer, stereoisomer or prodrug thereof, and
optionally a pharmaceutically acceptable excipient or carrier.

25. (canceled)

26. (canceled)

27. (canceled)

28. (canceled)

29. (canceled)

30. (canceled)

31. (canceled)

32. A method of degrading or reducing casein kinase 1 alpha (CK1α) in a subject in need thereof, the method comprising administering to the subject an effective amount of the pharmaceutical composition of claim 24.

33. A method of inhibiting casein kinase 1 alpha (CK1α) activity in a subject in need thereof, the method comprising administering to the subject an effective amount of the pharmaceutical composition of claim 24.

34. A method of preventing or treating blood cancer in a subject in need thereof, the method comprising administering to the subject an effective amount of the pharmaceutical composition of claim 24.

35. The method of claim 34, wherein the blood cancer is leukemia, lymphoma, or myeloma.

36. The method of claim 35, wherein the leukemia is selected from the group consisting of acute myeloid leukemia (AML), acute lymphoblastic leukemia or acute lymphocytic leukemia (ALL), T-cell acute lymphoblastic leukemia (T-ALL), B-cell acute lymphoblastic leukemia (B-ALL), chronic myeloid leukemia (CML), chronic lymphocytic leukemia (CLL), hairy cell leukemia (HCL), T-cell prolymphocytic leukemia (T-PLL), Large granular lymphocytic leukemia, adult T-cell leukemia, chronic eosinophilic leukemia (CEL) and myelodysplastic syndrome (MDS).

37. The method of claim 35, wherein the lymphoma is selected from the group consisting of Hodgkin's lymphoma, diffuse large B-cell lymphoma (DLBCL), follicular lymphoma, small lymphocytic lymphoma (SLL), mantle cell lymphoma (MCL), marginal zone lymphomas, Burkitt lymphoma, lymphoplasmacytic lymphoma, primary central nervous system (CNS) lymphoma and peripheral T-cell lymphoma.

38. The method of claim 35, the myeloma is selected from the group consisting of multiple myeloma, light chain myeloma, non-secretory myeloma, solitary plasmacytoma, extramedullary plasmacytoma, monoclonal gammopathy of undetermined significance (MGUS), smoldering multiple myeloma (SMM), immunoglobulin D (IgD) myeloma and immunoglobulin E (IgE) myeloma.

39. A compound of:
TABLE A

| Com- | | |
|---|---|---|
| pounds | | |
| No. | Structure | Name |
| 2 | 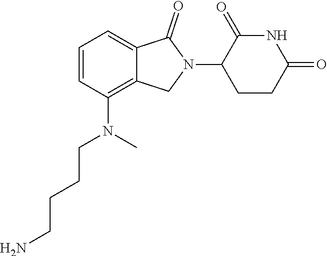 [SMILES: CN(CCCCN)c1cccc2c1CN(C1CCC(=O)NC1=O)C2=O] | 3-(4-((4- aminobutyl)(methyl)amino)-1- oxoisoindolin-2-yl)piperidine-2,6- dione |
| 3 | 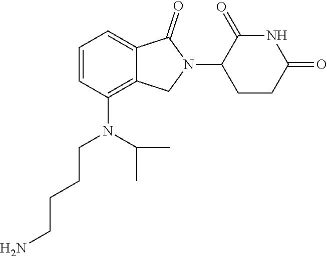 [SMILES: CC(C)N(CCCCN)c1cccc2c1CN(C1CCC(=O)NC1=O)C2=O] | 3-(4-((4- aminobutyl)(isopropyl)amino)-1- oxoisoindolin-2-yl)piperidine-2,6- dione |
| 4 | 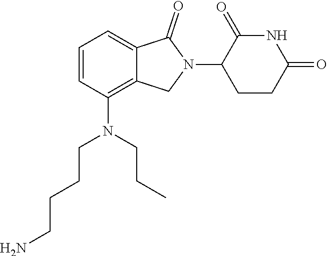 [SMILES: CCCN(CCCCN)c1cccc2c1CN(C1CCC(=O)NC1=O)C2=O] | 3-(4-((4- aminobutyl)(propyl)amino)-1- oxoisoindolin-2-yl)piperidine-2,6- dione |
| 46 | 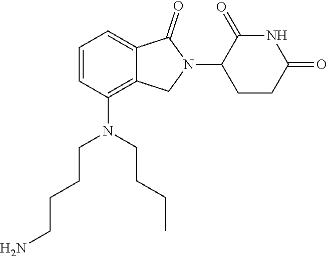 [SMILES: CCCCN(CCCCN)c1cccc2c1CN(C1CCC(=O)NC1=O)C2=O] | 3-(4-((4-aminobutyl)(butyl)amino)- 1-oxoisoindolin-2-yl)piperidine- 2,6-dione |
| 47 | 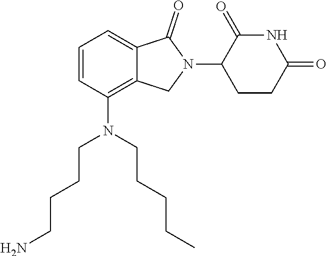 [SMILES: CCCCCN(CCCCN)c1cccc2c1CN(C1CCC(=O)NC1=O)C2=O] | 3-(4-((4- aminobutyl)(pentyl)amino)-1- oxoisoindolin-2-yl)piperidine-2,6- dione |
| 48 | 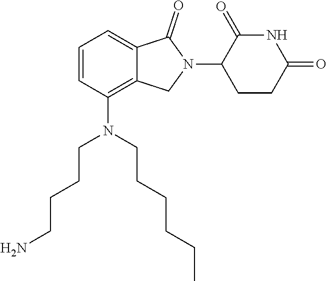 [SMILES: CCCCCCN(CCCCN)c1cccc2c1CN(C1CCC(=O)NC1=O)C2=O] | 3-(4-((4-aminobutyl)(hexyl)amino)- 1-oxoisoindolin-2-yl)piperidine- 2,6-dione |
| 49 | 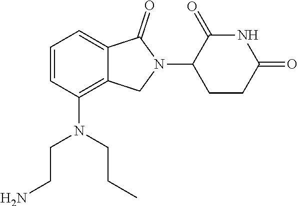 [SMILES: CCCN(CCN)c1cccc2c1CN(C1CCC(=O)NC1=O)C2=O] | 3-(4-((2- aminoethyl)(propyl)amino)-1- oxoisoindolin-2-yl)piperidine-2,6- dione |
| 50 | 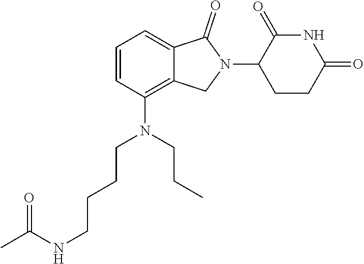 [SMILES: CCCN(CCCCNC(C)=O)c1cccc2c1CN(C1CCC(=O)NC1=O)C2=O] | N-(4-((2-(2,6-dioxopiperidin-3-yl)- 1-oxoisoindolin-4- yl)(propyl)amino)butyl)acetamide |
| 51 | 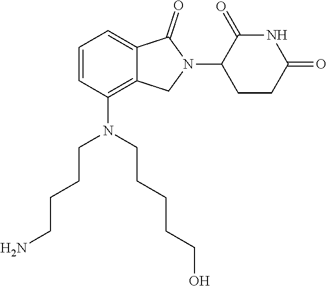 [SMILES: NCCCCN(CCCCCO)c1cccc2c1CN(C1CCC(=O)NC1=O)C2=O] | 3-(4-((4-aminobutyl)(5- hydroxypentyl)amino)-1- oxoisoindolin-2-yl)piperidine-2,6- dione |
| 52 | 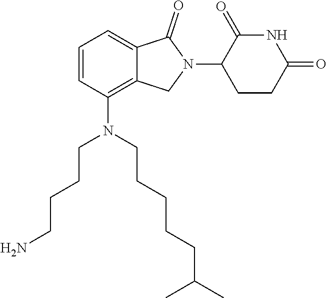 [SMILES: CC(C)CCCCCN(CCCCN)c1cccc2c1CN(C1CCC(=O)NC1=O)C2=O] | 3-(4-((4-aminobutyl)(6- methylheptyl)amino)-1- oxoisoindolin-2-yl)piperidine-2,6- dione |
| 53 | 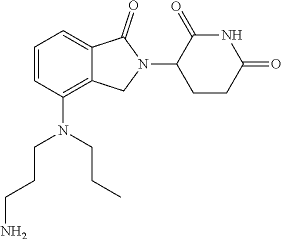 [SMILES: CCCN(CCCN)c1cccc2c1CN(C1CCC(=O)NC1=O)C2=O] | 3-(4-((3- aminopropyl)(propyl)amino)-1- oxoisoindolin-2-yl)piperidine-2,6- dione |
| 55 | 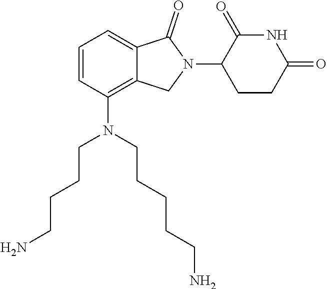 [SMILES: NCCCCCN(CCCCN)c1cccc2c1CN(C1CCC(=O)NC1=O)C2=O] | 3-(4-((4-aminobutyl)(5- aminopentyl)amino)-1- oxoisoindolin-2-yl)piperidine-2,6- dione |
| 56 | 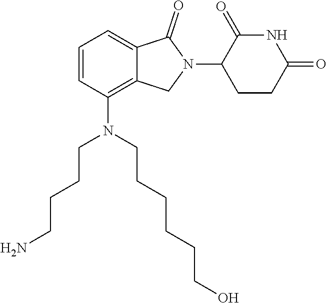 [SMILES: NCCCCN(CCCCCCO)c1cccc2c1CN(C1CCC(=O)NC1=O)C2=O] | 3-(4-((4-aminobutyl)(6- hydroxyhexyl)amino)-1- oxoisoindolin-2-yl)piperidine-2,6- dione |
| 57 | 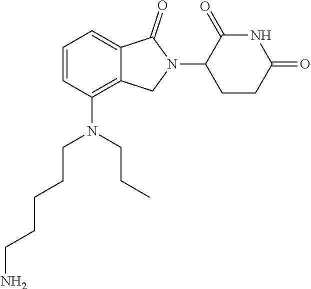 [SMILES: CCCN(CCCCCN)c1cccc2c1CN(C1CCC(=O)NC1=O)C2=O] | 3-(4-((5- aminopentyl)(propyl)amino)-1- oxoisoindolin-2-yl)piperidine-2,6- dione |
| 60 | 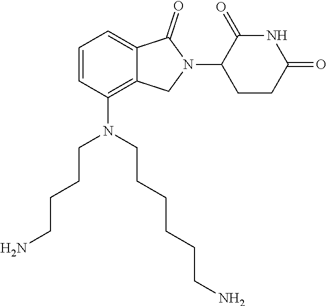 [SMILES: NCCCCCCN(CCCCN)c1cccc2c1CN(C1CCC(=O)NC1=O)C2=O] | 3-(4-((4-aminobutyl)(6- aminohexyl)amino)-1- oxoisoindolin-2-yl)piperidine-2,6- dione |
| 63 | 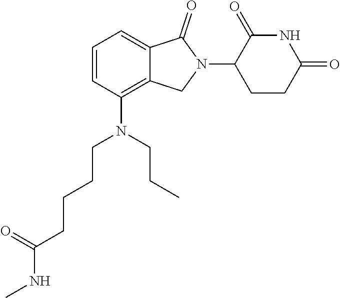 [SMILES: CCCN(CCCCC(=O)NC)c1cccc2c1CN(C1CCC(=O)NC1=O)C2=O] | 5-((2-(2,6-dioxopiperidin-3-yl)-1- oxoisoindolin-4-yl)(propyl)amino)- N-methylpentanamide |
| 66 | 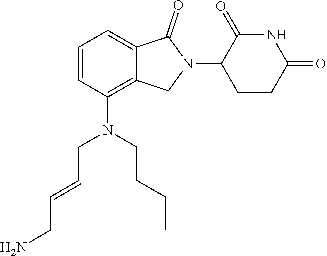 [SMILES: CCCCN(C/C=C/CN)c1cccc2c1CN(C1CCC(=O)NC1=O)C2=O] | (E)-3-(4-((4-aminobut-2-en-1- yl)(butyl)amino)-1-oxoisoindolin-2- yl)piperidine-2,6-dione |
| 67 | 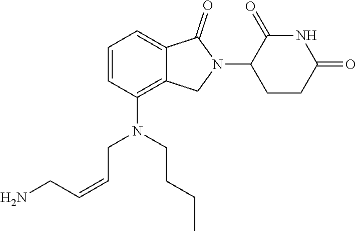 [SMILES: CCCCN(C/C=C\CN)c1cccc2c1CN(C1CCC(=O)NC1=O)C2=O] | (Z)-3-(4-((4-aminobut-2-en-1- yl)(butyl)amino)-1-oxoisoindolin-2- yl)piperidine-2,6-dione |
| 68 | 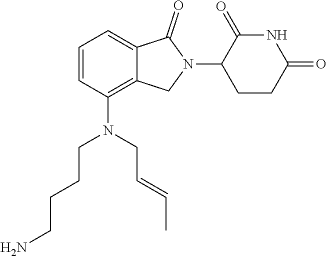 [SMILES: C/C=C/CN(CCCCN)c1cccc2c1CN(C1CCC(=O)NC1=O)C2=O] | (E)-3-(4-((4-aminobutyl)(but-2-en- 1-yl)amino)-1-oxoisoindolin-2- yl)piperidine-2,6-dione |
| 69 | 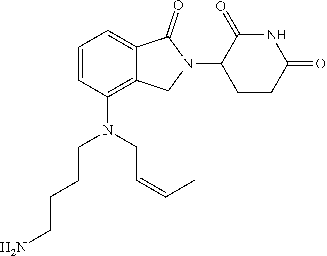 [SMILES: C/C=C\CN(CCCCN)c1cccc2c1CN(C1CCC(=O)NC1=O)C2=O] | (Z)-3-(4-((4-aminobutyl)(but-2-en- 1-yl)amino)-1-oxoisoindolin-2- yl)piperidine-2,6-dione |
| 80 | 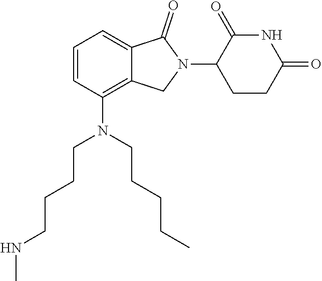 [SMILES: CCCCCN(CCCCNC)c1cccc2c1CN(C1CCC(=O)NC1=O)C2=O] | 3-(4-((4- (methylamino)butyl)(pentyl)amino)- 1-oxoisoindolin-2-yl)piperidine- 2,6-dione |
| 81 | 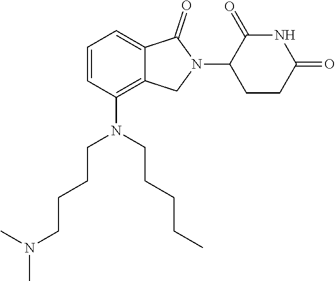 [SMILES: CCCCCN(CCCCN(C)C)c1cccc2c1CN(C1CCC(=O)NC1=O)C2=O] | 3-(4-((4- (dimethylamino)butyl)(pentyl)amino)- 1-oxoisoindolin-2-yl)piperidine- 2,6-dione |
| 82 | 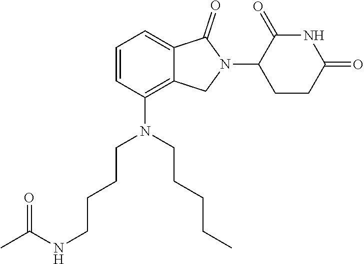 [SMILES: CCCCCN(CCCCNC(C)=O)c1cccc2c1CN(C1CCC(=O)NC1=O)C2=O] | N-(4-((2-(2,6-dioxopiperidin-3-yl)- 1-oxoisoindolin-4- yl)(pentyl)amino)butyl)acetamide |
| 87 | 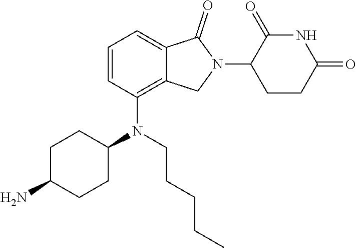 [SMILES: CCCCCN(c1cccc2c1CN(C1CCC(=O)NC1=O)C2=O)[C@H]1CC[C@@H](N)CC1] | 3-(4-(((1s,4s)-4- aminocyclohexyl)(pentyl)amino)-1- oxoisoindolin-2-yl)piperidine-2,6- dione |
| 88 | 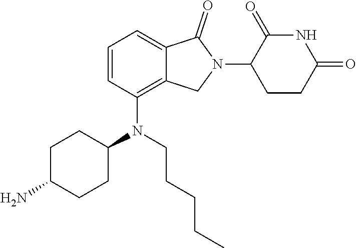 [SMILES: CCCCCN(c1cccc2c1CN(C1CCC(=O)NC1=O)C2=O)[C@H]1CC[C@H](N)CC1] | 3-(4-(((1r,4r)-4- aminocyclohexyl)(pentyl)amino)-1- oxoisoindolin-2-yl)piperidine-2,6- dione |
| 89 | 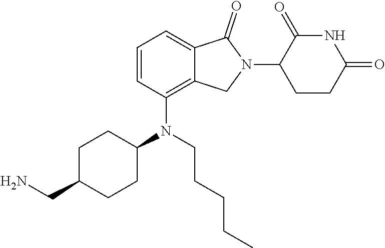 [SMILES: CCCCCN(c1cccc2c1CN(C1CCC(=O)NC1=O)C2=O)[C@H]1CC[C@@H](CN)CC1] | 3-(4-(((1s,4s)-4- (aminomethyl)cyclohexyl)(pentyl) amino)-1-oxoisoindolin-2- yl)piperidine-2,6-dione |
| 90 | 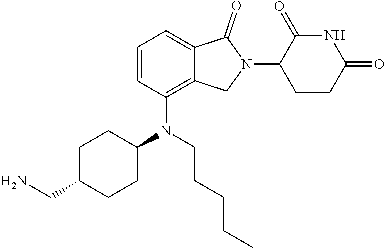 [SMILES: CCCCCN(c1cccc2c1CN(C1CCC(=O)NC1=O)C2=O)[C@H]1CC[C@H](CN)CC1] | 3-(4-(((1r,4r)-4- (aminomethyl)cyclohexyl)(pentyl) amino)-1-oxoisoindolin-2- yl)piperidine-2,6-dione |
| 91 | 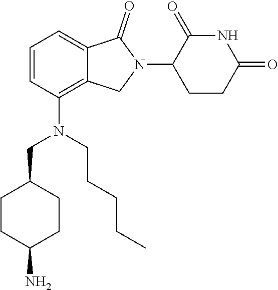 [SMILES: CCCCCN(C[C@H]1CC[C@@H](N)CC1)c1cccc2c1CN(C1CCC(=O)NC1=O)C2=O] | 3-(4-((((1s,4s)-4- aminocyclohexyl)methyl)(pentyl) amino)-1-oxoisoindolin-2- yl)piperidine-2,6-dione |
| 92 | 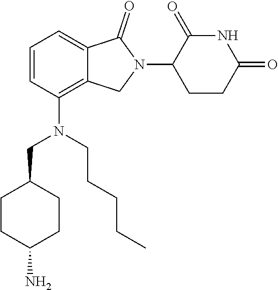 [SMILES: CCCCCN(C[C@H]1CC[C@H](N)CC1)c1cccc2c1CN(C1CCC(=O)NC1=O)C2=O] | 3-(4-((((1r,4r)-4- aminocyclohexyl)methyl)(pentyl) amino)-1-oxoisoindolin-2- yl)piperidine-2,6-dione |
| 93 | 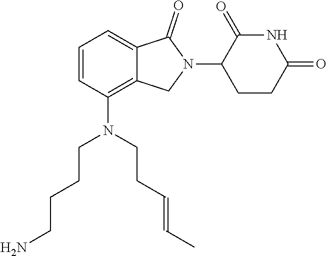 [SMILES: C/C=C/CCN(CCCCN)c1cccc2c1CN(C1CCC(=O)NC1=O)C2=O] | (E)-3-(4-((4-aminobutyl)(pent-3-en- 1-yl)amino)-1-oxoisoindolin-2- yl)piperidine-2,6-dione |
| 94 | 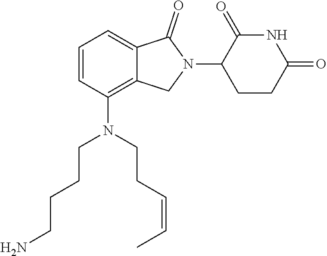 [SMILES: C/C=C\CCN(CCCCN)c1cccc2c1CN(C1CCC(=O)NC1=O)C2=O] | (Z)-3-(4-((4-aminobutyl)(pent-3-en- 1-yl)amino)-1-oxoisoindolin-2- yl)piperidine-2,6-dione |
| 95 | 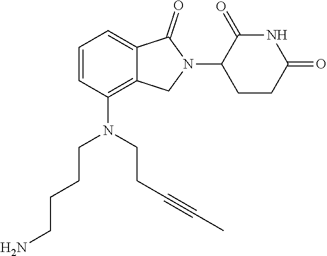 [SMILES: CC#CCCN(CCCCN)c1cccc2c1CN(C1CCC(=O)NC1=O)C2=O] | 3-(4-((4-aminobutyl)(pent-3-yn-1- yl)amino)-1-oxoisoindolin-2- yl)piperidine-2,6-dione |
| 96 | 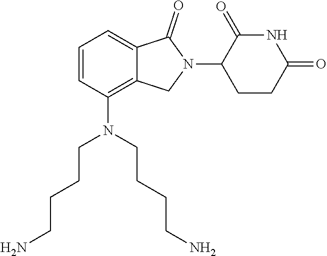 [SMILES: NCCCCN(CCCCN)c1cccc2c1CN(C1CCC(=O)NC1=O)C2=O] | 3-(4-(bis(4-aminobutyl)amino)-1- oxoisoindolin-2-yl)piperidine-2,6- dione |
| 97 | 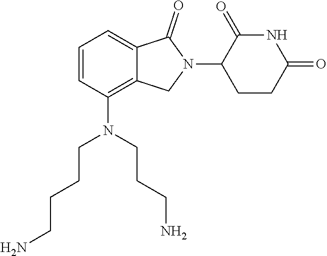 [SMILES: NCCCCN(CCCN)c1cccc2c1CN(C1CCC(=O)NC1=O)C2=O] | 3-(4-((4-aminobutyl)(3- aminopropyl)amino)-1- oxoisoindolin-2-yl)piperidine-2,6- dione |
| 102 | 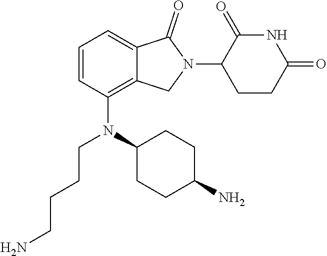 [SMILES: NCCCCN(c1cccc2c1CN(C1CCC(=O)NC1=O)C2=O)[C@H]1CC[C@@H](N)CC1] | 3-(4-((4-aminobutyl)((1s,4s)-4- aminocyclohexyl)amino)-1- oxoisoindolin-2-yl)piperidine-2,6- dione |
| 103 | 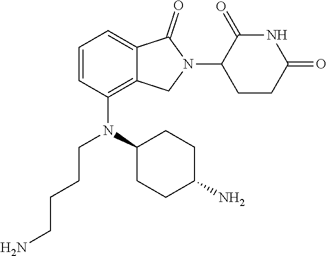 [SMILES: NCCCCN(c1cccc2c1CN(C1CCC(=O)NC1=O)C2=O)[C@H]1CC[C@H](N)CC1] | 3-(4-((4-aminobutyl)((1r,4r)-4- aminocyclohexyl)amino)-1- oxoisoindolin-2-yl)piperidine-2,6- dione |
| 104 | 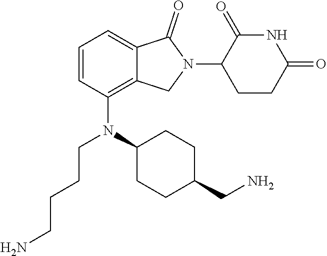 [SMILES: NCCCCN(c1cccc2c1CN(C1CCC(=O)NC1=O)C2=O)[C@H]1CC[C@@H](CN)CC1] | 3-(4-((4-aminobutyl)((1s,4s)-4- (aminomethyl)cyclohexyl)amino)- 1-oxoisoindolin-2-yl)piperidine- 2,6-dione |
| 105 | 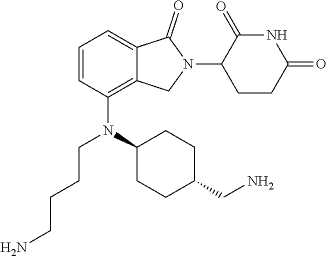 [SMILES: NCCCCN(c1cccc2c1CN(C1CCC(=O)NC1=O)C2=O)[C@H]1CC[C@H](CN)CC1] | 3-(4-((4-aminobutyl)((1r,4r)-4- (aminomethyl)cyclohexyl)amino)- 1-oxoisoindolin-2-yl)piperidine- 2,6-dione |
| 106 | 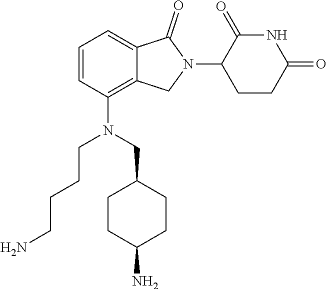 [SMILES: NCCCCN(C[C@H]1CC[C@@H](N)CC1)c1cccc2c1CN(C1CCC(=O)NC1=O)C2=O] | 3-(4-((4-aminobutyl)(((1s,4s)-4- aminocyclohexyl)methyl)amino)-1- oxoisoindolin-2-yl)piperidine-2,6- dione |
| 107 | 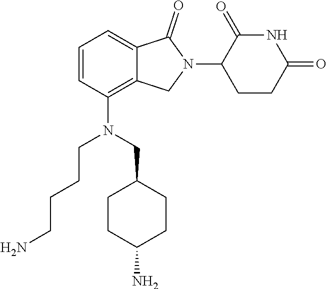 [SMILES: NCCCCN(C[C@H]1CC[C@H](N)CC1)c1cccc2c1CN(C1CCC(=O)NC1=O)C2=O] | 3-(4-((4-aminobutyl)(((1r,4r)-4- aminocyclohexyl)methyl)amino)-1- oxoisoindolin-2-yl)piperidine-2,6- dione |
| 108 | 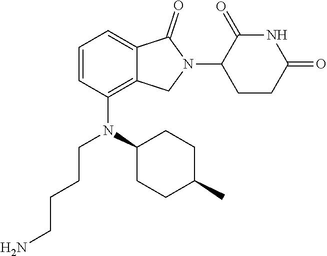 [SMILES: C[C@H]1CC[C@@H](N(CCCCN)c2cccc3c2CN(C2CCC(=O)NC2=O)C3=O)CC1] | 3-(4-((4-aminobutyl)((1s,4s)-4- methylcyclohexyl)amino)-1- oxoisoindolin-2-yl)piperidine-2,6- dione |
| 109 | 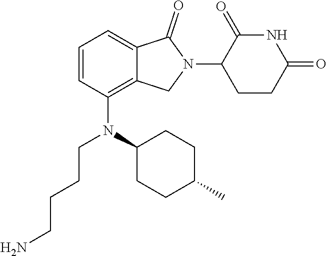 [SMILES: C[C@H]1CC[C@H](N(CCCCN)c2cccc3c2CN(C2CCC(=O)NC2=O)C3=O)CC1] | 3-(4-((4-aminobutyl)((1r,4r)-4- methylcyclohexyl)amino)-1- oxoisoindolin-2-yl)piperidine-2,6- dione |
| 115 | 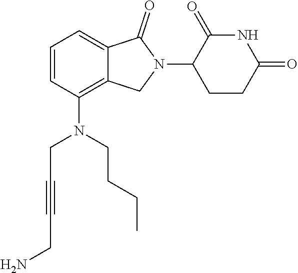 [SMILES: CCCCN(CC#CCN)c1cccc2c1CN(C1CCC(=O)NC1=O)C2=O] | 3-(4-((4-aminobut-2-yn-1- yl)(butyl)amino)-1-oxoisoindolin-2- yl)piperidine-2,6-dione |
| 116 | 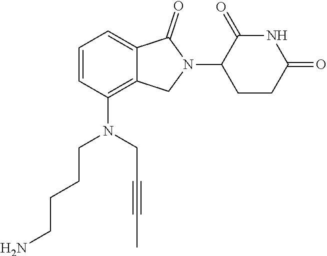 [SMILES: CC#CCN(CCCCN)c1cccc2c1CN(C1CCC(=O)NC1=O)C2=O] | 3-(4-((4-aminobutyl)(but-2-yn-1- yl)amino)-1-oxoisoindolin-2- yl)piperidine-2,6-dione |
| 123 | 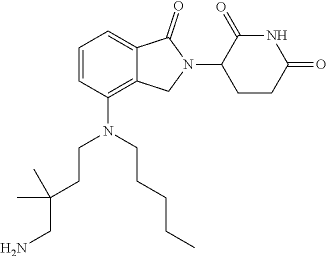 [SMILES: CCCCCN(CCC(C)(C)CN)c1cccc2c1CN(C1CCC(=O)NC1=O)C2=O] | 3-(4-((4-amino-3,3- dimethylbutyl)(pentyl)amino)-1- oxoisoindolin-2-yl)piperidine-2,6- dione |
| 124 | 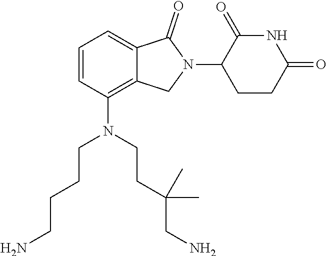 [SMILES: CC(C)(CN)CCN(CCCCN)c1cccc2c1CN(C1CCC(=O)NC1=O)C2=O] | 3-(4-((4-amino-3,3- dimethylbutyl)(4- aminobutyl)amino)-1- oxoisoindolin-2-yl)piperidine-2,6- dione |
| 125 | 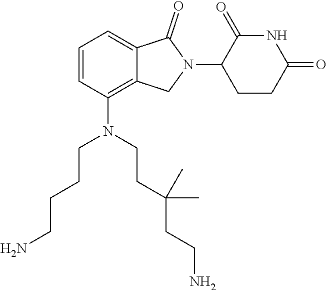 [SMILES: CC(C)(CCN)CCN(CCCCN)c1cccc2c1CN(C1CCC(=O)NC1=O)C2=O] | 3-(4-((5-amino-3,3- dimethylpentyl)(4- aminobutyl)amino)-1- oxoisoindolin-2-yl)piperidine-2,6- dione |
| 126 | 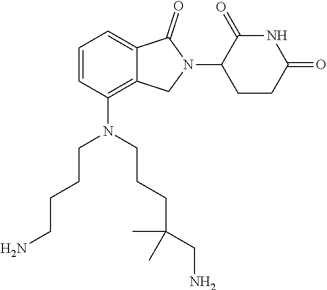 [SMILES: CC(C)(CN)CCCN(CCCCN)c1cccc2c1CN(C1CCC(=O)NC1=O)C2=O] | 3-(4-((5-amino-4,4- dimethylpentyl)(4- aminobutyl)amino)-1- oxoisoindolin-2-yl)piperidine-2,6- dione |
| 143 | 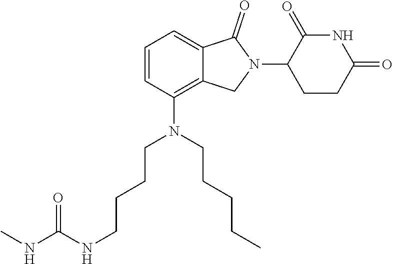 [SMILES: CCCCCN(CCCCNC(=O)NC)c1cccc2c1CN(C1CCC(=O)NC1=O)C2=O] | 1-(4-((2-(2,6-dioxopiperidin-3-yl)- 1-oxoisoindolin-4- yl)(pentyl)amino)butyl)-3- methylurea |
| 148 | 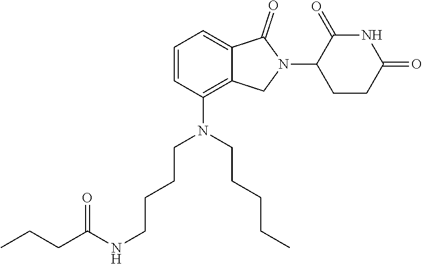 [SMILES: CCCCCN(CCCCNC(=O)CCC)c1cccc2c1CN(C1CCC(=O)NC1=O)C2=O] | N-(4-((2-(2,6-dioxopiperidin-3-yl)- 1-oxoisoindolin-4- yl)(pentyl)amino)butyl)butyramide |
| 149 | 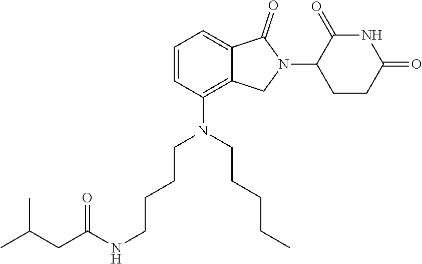 [SMILES: CCCCCN(CCCCNC(=O)CC(C)C)c1cccc2c1CN(C1CCC(=O)NC1=O)C2=O] | N-(4-((2-(2,6-dioxopiperidin-3-yl)- 1-oxoisoindolin-4- yl)(pentyl)amino)butyl)-3- methylbutanamide |
| 150 | 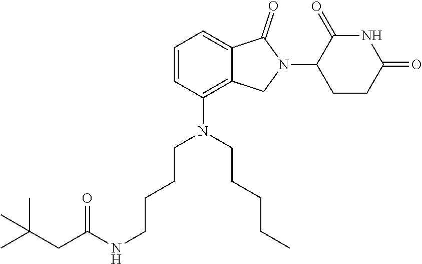 [SMILES: CCCCCN(CCCCNC(=O)CC(C)(C)C)c1cccc2c1CN(C1CCC(=O)NC1=O)C2=O] | N-(4-((2-(2,6-dioxopiperidin-3-yl)- 1-oxoisoindolin-4- yl)(pentyl)amino)butyl)-3,3- dimethylbutanamide |
| 151 | 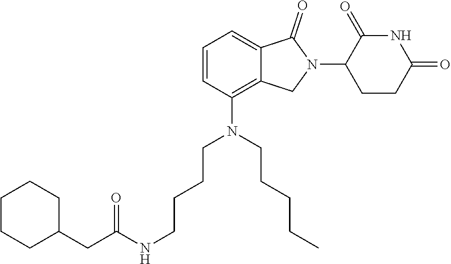 [SMILES: CCCCCN(CCCCNC(=O)CC1CCCCC1)c1cccc2c1CN(C1CCC(=O)NC1=O)C2=O] | 2-cyclohexyl-N-(4-((2-(2,6- dioxopiperidin-3-yl)-1- oxoisoindolin-4- yl)(pentyl)amino)butyl)acetamide |
| 163 | 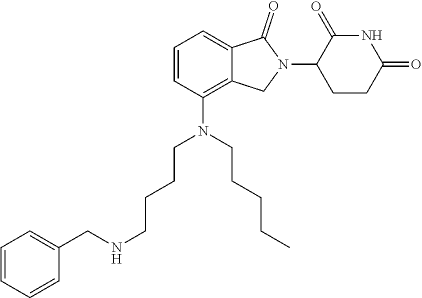 [SMILES: CCCCCN(CCCCNCc1ccccc1)c1cccc2c1CN(C1CCC(=O)NC1=O)C2=O] | 3-(4-((4- (benzylamino)butyl)(pentyl)amino)- 1-oxoisoindolin-2-yl)piperidine- 2,6-dione |
| 164 | 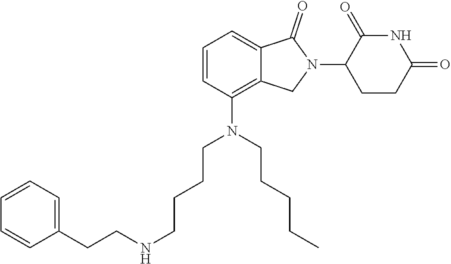 [SMILES: CCCCCN(CCCCNCCc1ccccc1)c1cccc2c1CN(C1CCC(=O)NC1=O)C2=O] | 3-(1-oxo-4-(pentyl(4- (phenethylamino)butyl)amino) isoindolin-2-yl)piperidine-2,6-dione |
| 165 | 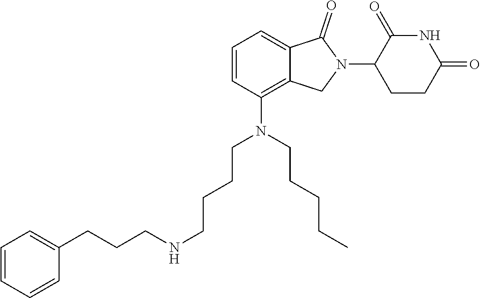 [SMILES: CCCCCN(CCCCNCCCc1ccccc1)c1cccc2c1CN(C1CCC(=O)NC1=O)C2=O] | 3-(1-oxo-4-(pentyl(4-((3- phenylpropyl)amino)butyl)amino) isoindolin-2-yl)piperidine-2,6-dione |
| 166 | 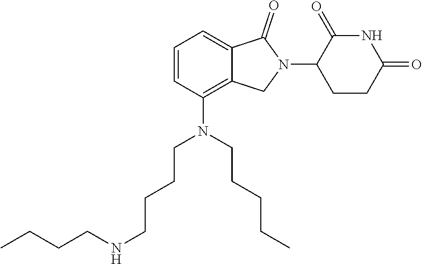 [SMILES: CCCCCN(CCCCNCCCC)c1cccc2c1CN(C1CCC(=O)NC1=O)C2=O] | 3-(4-((4- (butylamino)butyl)(pentyl)amino)- 1-oxoisoindolin-2-yl)piperidine- 2,6-dione |
| 167 | 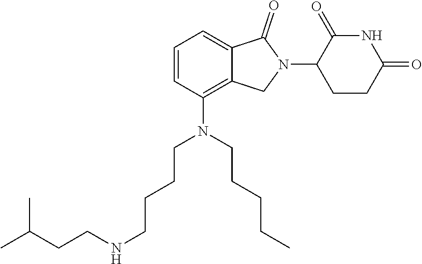 [SMILES: CCCCCN(CCCCNCCC(C)C)c1cccc2c1CN(C1CCC(=O)NC1=O)C2=O] | 3-(4-((4-(isopentylamino) butyl)(pentyl)amino)- 1-oxoisoindolin-2-yl)piperidine- 2,6-dione |
| 168 | 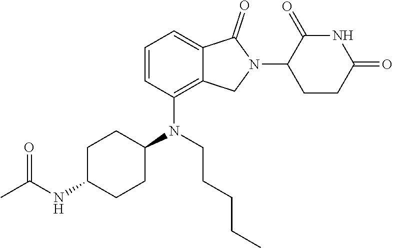 [SMILES: CCCCCN(c1cccc2c1CN(C1CCC(=O)NC1=O)C2=O)[C@H]1CC[C@H](NC(C)=O)CC1] | N-((1r,4r)-4-((2-(2,6- dioxopiperidin-3-yl)-1- oxoisoindolin-4- yl)(pentyl)amino)cyclohexyl) acetamide |
| 169 | 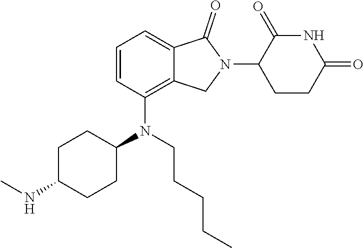 [SMILES: CCCCCN(c1cccc2c1CN(C1CCC(=O)NC1=O)C2=O)[C@H]1CC[C@H](NC)CC1] | 3-(4-(((1r,4r)-4- (methylamino)cyclohexyl)(pentyl) amino)-1-oxoisoindolin-2- yl)piperidine-2,6-dione |
| 181 | 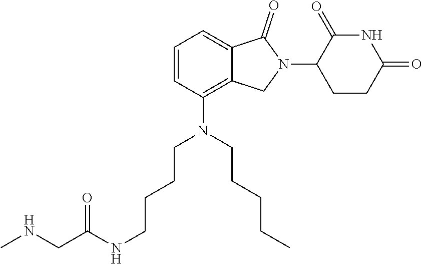 [SMILES: CCCCCN(CCCCNC(=O)CNC)c1cccc2c1CN(C1CCC(=O)NC1=O)C2=O] | N-(4-((2-(2,6-dioxopiperidin-3-yl)- 1-oxoisoindolin-4- yl)(pentyl)amino)butyl)-2- (methylamino)acetamide |
| 223 | 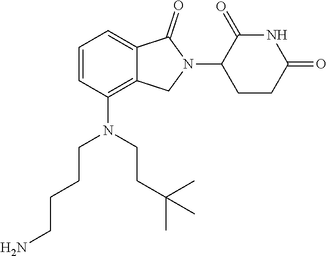 [SMILES: CC(C)(C)CCN(CCCCN)c1cccc2c1CN(C1CCC(=O)NC1=O)C2=O] | 3-(4-((4-aminobutyl)(3,3- dimethylbutyl)amino)-1- oxoisoindolin-2-yl)piperidine-2,6- dione |
| 235 | 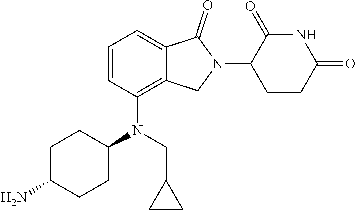 [SMILES: N[C@H]1CC[C@H](N(CC2CC2)c2cccc3c2CN(C2CCC(=O)NC2=O)C3=O)CC1] | 3-(4-(((1r,4r)-4-amino- cyclohexyl)(cyclopropylmethyl) amino)-1-oxoisoindolin-2- yl)piperidine-2,6-dione |
| 236 | 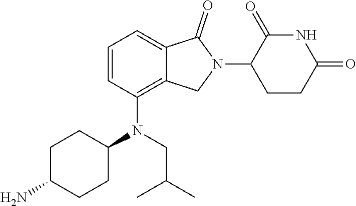 [SMILES: CC(C)CN(c1cccc2c1CN(C1CCC(=O)NC1=O)C2=O)[C@H]1CC[C@H](N)CC1] | 3-(4-(((1r,4r)-4- aminocyclohexyl)(isobutyl)amino)- 1-oxoisoindolin-2-yl)piperidine- 2,6-dione |
| 237 | 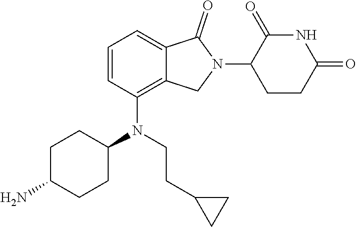 [SMILES: N[C@H]1CC[C@H](N(CCC2CC2)c2cccc3c2CN(C2CCC(=O)NC2=O)C3=O)CC1] | 3-(4-(((1r,4r)-4- aminocyclohexyl)(2- cyclopropylethyl)amino)-1- oxoisoindolin-2-yl)piperidine-2,6- dione |
| 238 | 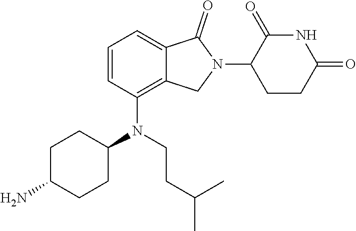 [SMILES: CC(C)CCN(c1cccc2c1CN(C1CCC(=O)NC1=O)C2=O)[C@H]1CC[C@H](N)CC1] | 3-(4-(((1r,4r)-4- aminocyclohexyl)(isopentyl)amino)- 1-oxoisoindolin-2-yl)piperidine- 2,6-dione |
| 239 | 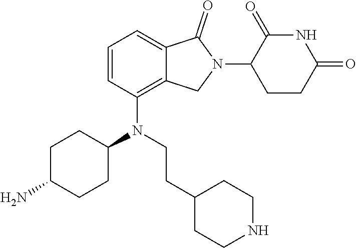 [SMILES: N[C@H]1CC[C@H](N(CCC2CCNCC2)c2cccc3c2CN(C2CCC(=O)NC2=O)C3=O)CC1] | 3-(4-(((1r,4r)-4- aminocyclohexyl)(2-(piperidin-4- yl)ethyl)amino)-1-oxoisoindolin-2- yl)piperidine-2,6-dione |
| 240 | 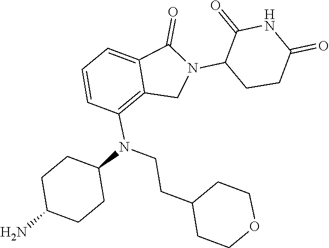 [SMILES: N[C@H]1CC[C@H](N(CCC2CCOCC2)c2cccc3c2CN(C2CCC(=O)NC2=O)C3=O)CC1] | 3-(4-(((1r,4r)-4- aminocyclohexyl)(2-(tetrahydro- 2H-pyran-4-yl)ethyl)amino)-1- oxoisoindolin-2-yl)piperidine-2,6- dione |
| 241 | 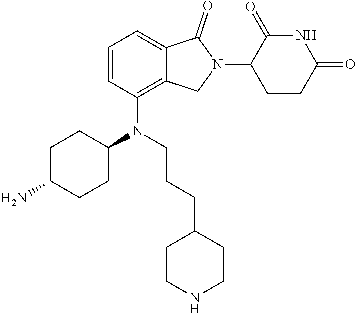 [SMILES: N[C@H]1CC[C@H](N(CCCC2CCNCC2)c2cccc3c2CN(C2CCC(=O)NC2=O)C3=O)CC1] | 3-(4-(((1r,4r)-4- aminocyclohexyl)(3-(piperidin-4- yl)propyl)amino)-1-oxoisoindolin- 2-yl)piperidine-2,6-dione |
| 242 | 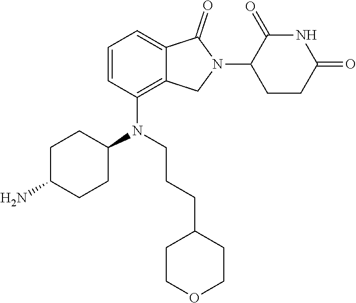 [SMILES: N[C@H]1CC[C@H](N(CCCC2CCOCC2)c2cccc3c2CN(C2CCC(=O)NC2=O)C3=O)CC1] | 3-(4-(((1r,4r)-4- aminocyclohexyl)(3-(tetrahydro- 2H-pyran-4-yl)propyl)amino)-1- oxoisoindolin-2-yl)piperidine-2,6- dione |
| 243 | 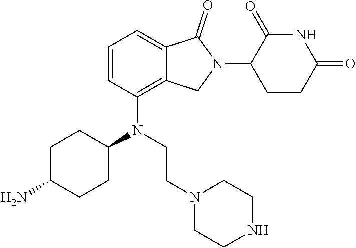 [SMILES: N[C@H]1CC[C@H](N(CCN2CCNCC2)c2cccc3c2CN(C2CCC(=O)NC2=O)C3=O)CC1] | 3-(4-(((1r,4r)-4- aminocyclohexyl)(2-(piperazin-1- yl)ethyl)amino)-1-oxoisoindolin-2- yl)piperidine-2,6-dione |
| 244 | 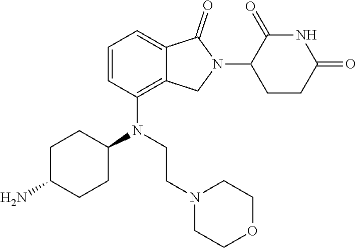 [SMILES: N[C@H]1CC[C@H](N(CCN2CCOCC2)c2cccc3c2CN(C2CCC(=O)NC2=O)C3=O)CC1] | 3-(4-(((1r,4r)-4- aminocyclohexyl)(2- morpholinoethyl)amino)-1- oxoisoindolin-2-yl)piperidine-2,6- dione |
| 245 | 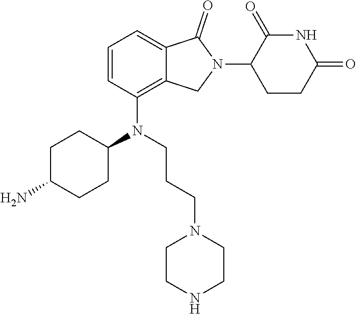 [SMILES: N[C@H]1CC[C@H](N(CCCN2CCNCC2)c2cccc3c2CN(C2CCC(=O)NC2=O)C3=O)CC1] | 3-(4-(((1r,4r)-4- aminocyclohexyl)(3-(piperazin-1- yl)propyl)amino)-1-oxoisoindolin- 2-yl)piperidine-2,6-dione |
| 246 | 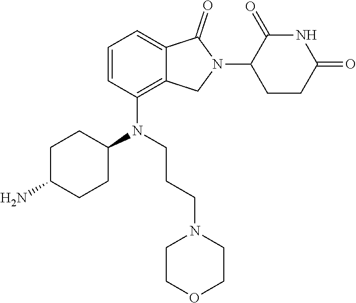 [SMILES: N[C@H]1CC[C@H](N(CCCN2CCOCC2)c2cccc3c2CN(C2CCC(=O)NC2=O)C3=O)CC1] | 3-(4-(((1r,4r)-4- aminocyclohexyl)(3- morpholinopropyl)amino)-1- oxoisoindolin-2-yl)piperidine-2,6- dione |
| 247 | 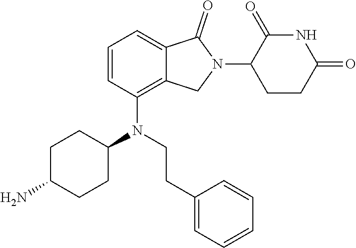 [SMILES: N[C@H]1CC[C@H](N(CCc2ccccc2)c2cccc3c2CN(C2CCC(=O)NC2=O)C3=O)CC1] | 3-(4-(((1r,4r)-4-amino- cyclohexyl)(phenethyl)amino)- 1-oxoisoindolin-2-yl)piperidine- 2,6-dione |
| 251 | 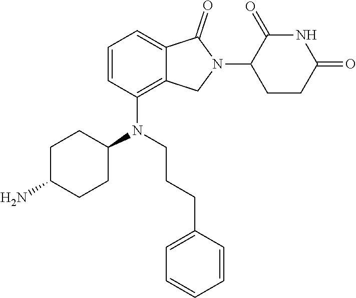 [SMILES: N[C@H]1CC[C@H](N(CCCc2ccccc2)c2cccc3c2CN(C2CCC(=O)NC2=O)C3=O)CC1] | 3-(4-(((1r,4r)-4- aminocyclohexyl)(3- phenylpropyl)amino)-1- oxoisoindolin-2-yl)piperidine-2,6- dione |
| 255 | 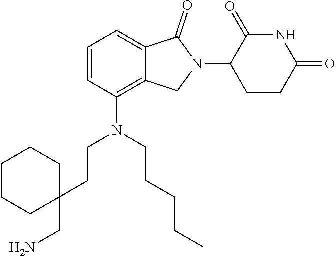 [SMILES: CCCCCN(CCC1(CN)CCCCC1)c1cccc2c1CN(C1CCC(=O)NC1=O)C2=O] | 3-(4-((2-(1- (aminomethyl)cyclohexyl)ethyl) (pentyl)amino)-1-oxoisoindolin-2- yl)piperidine-2,6-dione |
| 267 | 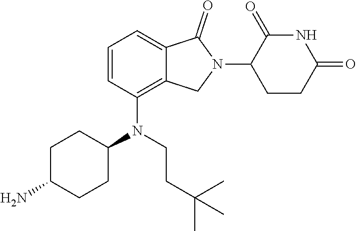 [SMILES: CC(C)(C)CCN(c1cccc2c1CN(C1CCC(=O)NC1=O)C2=O)[C@H]1CC[C@H](N)CC1] | 3-(4-(((1r,4r)-4- aminocyclohexyl)(3,3- dimethylbutyl)amino)-1- oxoisoindolin-2-yl)piperidine-2,6- dione |
| 268 | 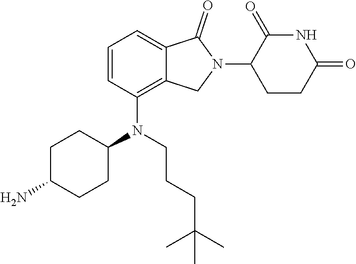 [SMILES: CC(C)(C)CCCN(c1cccc2c1CN(C1CCC(=O)NC1=O)C2=O)[C@H]1CC[C@H](N)CC1] | 3-(4-(((1r,4r)-4- aminocyclohexyl)(4,4- dimethylpentyl)amino)-1- oxoisoindolin-2-yl)piperidine-2,6- dione |
| 273 | 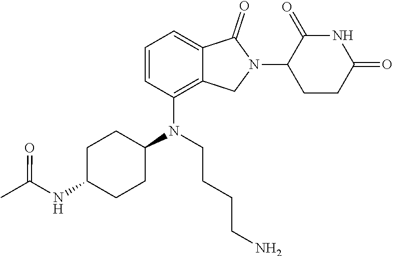 [SMILES: CC(=O)N[C@H]1CC[C@H](N(CCCCN)c2cccc3c2CN(C2CCC(=O)NC2=O)C3=O)CC1] | N-((1r,4r)-4-((4-aminobutyl)(2- (2,6-dioxopiperidin-3-yl)-1- oxoisoindolin-4- yl)amino)cyclohexyl)acetamide |
| 305 | 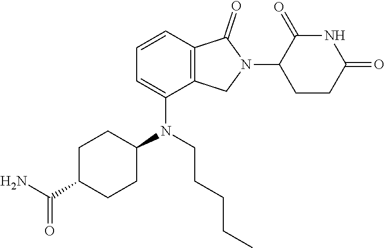 [SMILES: CCCCCN(c1cccc2c1CN(C1CCC(=O)NC1=O)C2=O)[C@H]1CC[C@H](C(N)=O)CC1] | (1r,4r)-4-((2-(2,6-dioxopiperidin-3- yl)-1-oxoisoindolin-4- yl)(pentyl)amino)cyclohexane- carboxamide |
| 306 | 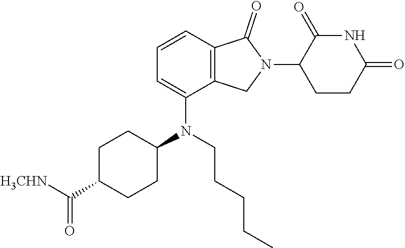 | (1r,4r)-4-((2-(2,6-dioxopiperidin-3- yl)-1-oxoisoindolin-4- yl)(pentyl)amino)-N- methylcyclohexanecarboxamide |
| 307 | 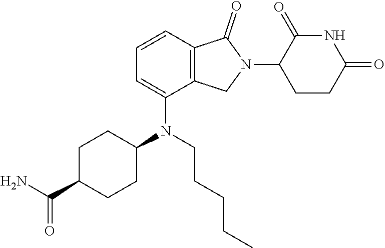 [SMILES: CCCCCN(c1cccc2c1CN(C1CCC(=O)NC1=O)C2=O)[C@H]1CC[C@@H](C(N)=O)CC1] | (1s,4s)-4-((2-(2,6-dioxopiperidin-3- yl)-1-oxoisoindolin-4- yl)(pentyl)amino)cyclohexane- carboxamide |
| 308 | 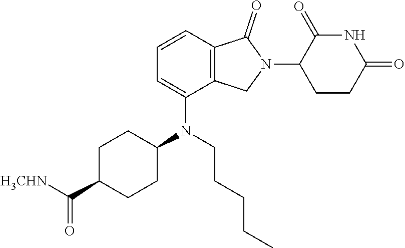 | (1s,4s)-4-((2-(2,6-dioxopiperidin-3- yl)-1-oxoisoindolin-4- yl)(pentyl)amino)-N- methylcyclohexanecarboxamide |
| 309 | 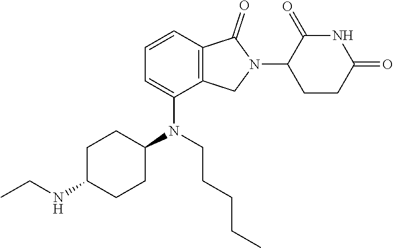 [SMILES: CCCCCN(c1cccc2c1CN(C1CCC(=O)NC1=O)C2=O)[C@H]1CC[C@H](NCC)CC1] | 3-(4-(((1r,4r)-4- (ethylamino)cyclohexyl)(pentyl) amino)-1-oxoisoindolin-2- yl)piperidine-2,6-dione |
| 310 | 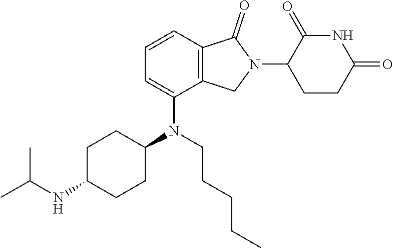 [SMILES: CCCCCN(c1cccc2c1CN(C1CCC(=O)NC1=O)C2=O)[C@H]1CC[C@H](NC(C)C)CC1] | 3-(4-(((1r,4r)-4- (isopropylamino)cyclohexyl) (pentyl)amino)-1-oxoisoindolin-2- yl)piperidine-2,6-dione |
| 311 | 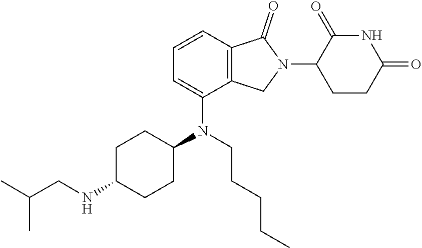 [SMILES: CCCCCN(c1cccc2c1CN(C1CCC(=O)NC1=O)C2=O)[C@H]1CC[C@H](NCC(C)C)CC1] | 3-(4-(((1r,4r)-4- (isobutylamino)cyclohexyl)(pentyl) amino)-1-oxoisoindolin-2- yl)piperidine-2,6-dione |
| 312 | 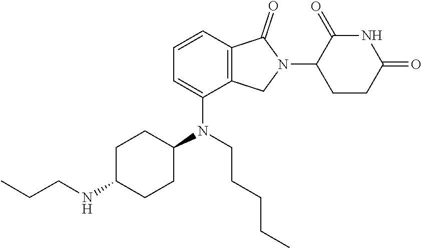 [SMILES: CCCCCN(c1cccc2c1CN(C1CCC(=O)NC1=O)C2=O)[C@H]1CC[C@H](NCCC)CC1] | 3-(1-oxo-4-(pentyl((1r,4r)-4- (propylamino)cyclohexyl)amino) isoindolin-2-yl)piperidine-2,6-dione |
| 313 | 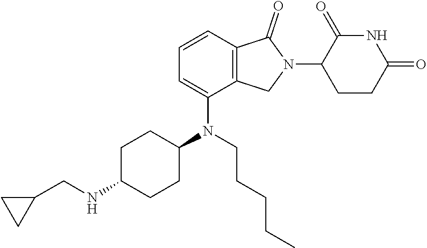 [SMILES: CCCCCN(c1cccc2c1CN(C1CCC(=O)NC1=O)C2=O)[C@H]1CC[C@H](NCC2CC2)CC1] | 3-(4-(((1r,4r)-4- ((cyclopropylmethyl)amino) cyclohexyl)(pentyl)amino)-1- oxoisoindolin-2-yl)piperidine-2,6- dione |
| 314 | 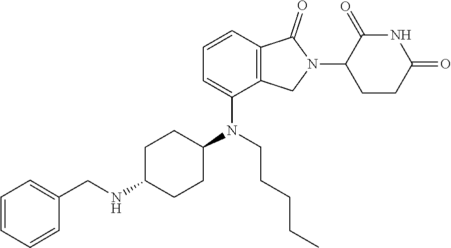 [SMILES: CCCCCN(c1cccc2c1CN(C1CCC(=O)NC1=O)C2=O)[C@H]1CC[C@H](NCc2ccccc2)CC1] | 3-(4-(((1r,4r)-4- (benzylamino)cyclohexyl)(pentyl) amino)-1-oxoisoindolin-2- yl)piperidine-2,6-dione |
| 323 | 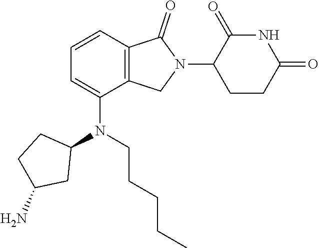 [SMILES: CCCCCN(c1cccc2c1CN(C1CCC(=O)NC1=O)C2=O)[C@@H]1CC[C@@H](N)C1] | 3-(4-(((1R,3R)-3- aminocyclopentyl)(pentyl)amino)- 1-oxoisoindolin-2-yl)piperidine- 2,6-dione |
| 324 | 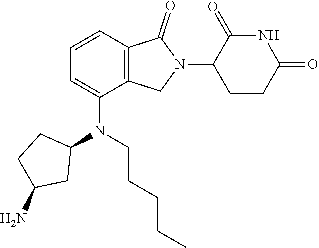 [SMILES: CCCCCN(c1cccc2c1CN(C1CCC(=O)NC1=O)C2=O)[C@@H]1CC[C@H](N)C1] | 3-(4-(((1R,3S)-3- aminocyclopentyl)(pentyl)amino)- 1-oxoisoindolin-2-yl)piperidine- 2,6-dione |
| 325 | 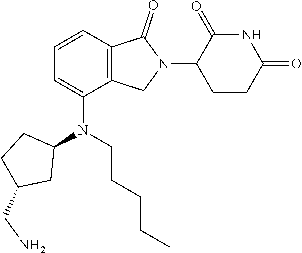 [SMILES: CCCCCN(c1cccc2c1CN(C1CCC(=O)NC1=O)C2=O)[C@@H]1CC[C@@H](CN)C1] | 3-(4-(((1R,3R)-3- (aminomethyl)cyclopentyl)(pentyl) amino)-1-oxoisoindolin-2- yl)piperidine-2,6-dione |
| 325A | 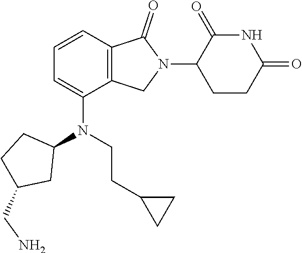 [SMILES: NC[C@@H]1CC[C@@H](N(CCC2CC2)c2cccc3c2CN(C2CCC(=O)NC2=O)C3=O)C1] | 3-(4-(((1R,3R)-3- (aminomethyl)cyclopentyl)(2- cyclopropylethyl)amino)-1- oxoisoindolin-2-yl)piperidine-2,6- dione |
| 326 | 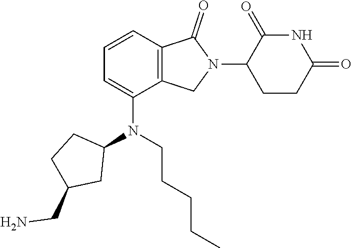 [SMILES: CCCCCN(c1cccc2c1CN(C1CCC(=O)NC1=O)C2=O)[C@@H]1CC[C@H](CN)C1] | 3-(4-(((1R,3S)-3- (aminomethyl)cyclopentyl)(pentyl) amino)-1-oxoisoindolin-2- yl)piperidine-2,6-dione |
| 326A | 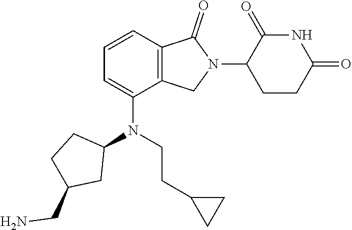 [SMILES: NC[C@H]1CC[C@@H](N(CCC2CC2)c2cccc3c2CN(C2CCC(=O)NC2=O)C3=O)C1] | 3-(4-(((1R,3S)-3- (aminomethyl)cyclopentyl)(2- cyclopropylethyl)amino)-1- oxoisoindolin-2-yl)piperidine-2,6- dione |
| 327 | 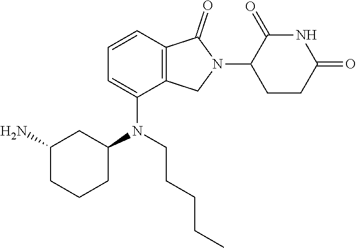 [SMILES: CCCCCN(c1cccc2c1CN(C1CCC(=O)NC1=O)C2=O)[C@H]1CCC[C@H](N)C1] | 3-(4-(((1S,3S)-3- aminocyclohexyl)(pentyl)amino)-1- oxoisoindolin-2-yl)piperidine-2,6- dione |
| 328 | 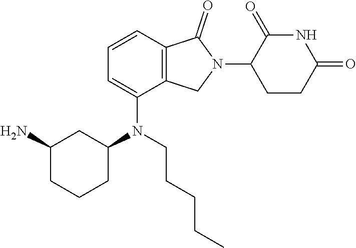 [SMILES: CCCCCN(c1cccc2c1CN(C1CCC(=O)NC1=O)C2=O)[C@H]1CCC[C@@H](N)C1] | 3-(4-(((1S,3R)-3- aminocyclohexyl)(pentyl)amino)-1- oxoisoindolin-2-yl)piperidine-2,6- dione |
| 329 | 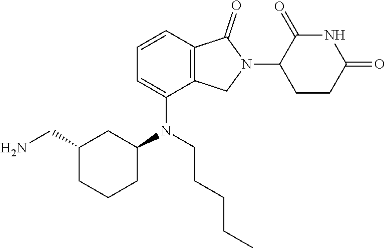 [SMILES: CCCCCN(c1cccc2c1CN(C1CCC(=O)NC1=O)C2=O)[C@H]1CCC[C@H](CN)C1] | 3-(4-(((1S,3S)-3- (aminomethyl)cyclohexyl)(pentyl) amino)-1-oxoisoindolin-2- yl)piperidine-2,6-dione |
| 330 | 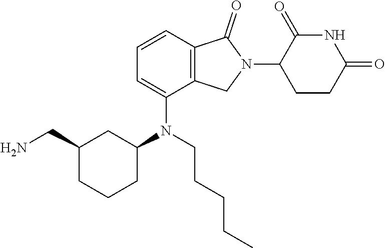 [SMILES: CCCCCN(c1cccc2c1CN(C1CCC(=O)NC1=O)C2=O)[C@H]1CCC[C@@H](CN)C1] | 3-(4-(((1S,3R)-3- (aminomethyl)cyclohexyl)(pentyl) amino)-1-oxoisoindolin-2- yl)piperidine-2,6-dione |
| 331 | 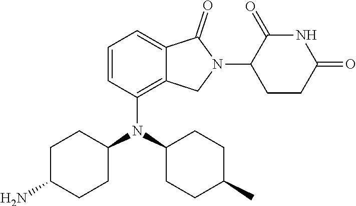 [SMILES: C[C@H]1CC[C@@H](N(c2cccc3c2CN(C2CCC(=O)NC2=O)C3=O)[C@H]2CC[C@H](N)CC2)CC1] | 3-(4-(((1r,4R)-4- aminocyclohexyl)((1s,4S)-4- methylcyclohexyl)amino)-1- oxoisoindolin-2-yl)piperidine-2,6- dione |
| 332 | 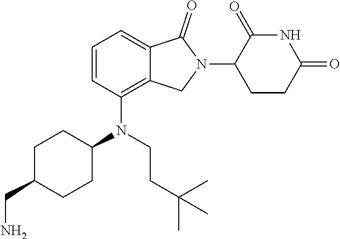 [SMILES: CC(C)(C)CCN(c1cccc2c1CN(C1CCC(=O)NC1=O)C2=O)[C@H]1CC[C@@H](CN)CC1] | 3-(4-(((1s,4s)-4- (aminomethyl)cyclohexyl)(3,3- dimethylbutyl)amino)-1- oxoisoindolin-2-yl)piperidine-2,6- dione |
| 333 | 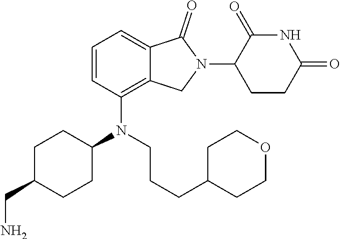 [SMILES: NC[C@H]1CC[C@@H](N(CCCC2CCOCC2)c2cccc3c2CN(C2CCC(=O)NC2=O)C3=O)CC1] | 3-(4-(((1s,4s)-4- (aminomethyl)cyclohexyl)(3- (tetrahydro-2H-pyran-4- yl)propyl)amino)-1-oxoisoindolin- 2-yl)piperidine-2,6-dione |
| 334 | 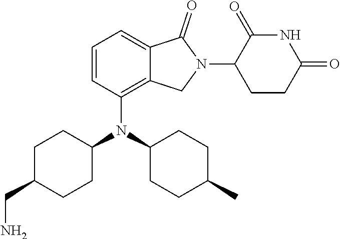 [SMILES: C[C@H]1CC[C@@H](N(c2cccc3c2CN(C2CCC(=O)NC2=O)C3=O)[C@H]2CC[C@@H](CN)CC2)CC1] | 3-(4-(((1s,4S)-4- (aminomethyl)cyclohexyl)((1s,4S)- 4-methylcyclohexyl)amino)-1- oxoisoindolin-2-yl)piperidine-2,6- dione |
| 340 | 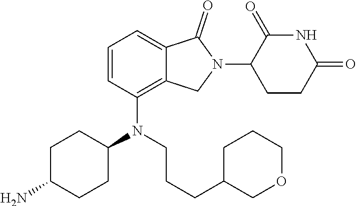 [SMILES: N[C@H]1CC[C@H](N(CCCC2CCCOC2)c2cccc3c2CN(C2CCC(=O)NC2=O)C3=O)CC1] | 3-(4-(((1r,4r)-4- aminocyclohexyl)(3-(tetrahydro- 2H-pyran-3-yl)propyl)amino)-1- oxoisoindolin-2-yl)piperidine-2,6- dione |
| 341 | 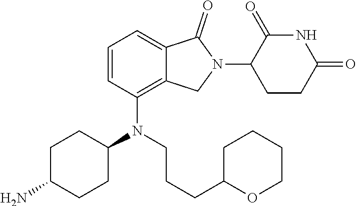 [SMILES: N[C@H]1CC[C@H](N(CCCC2CCCCO2)c2cccc3c2CN(C2CCC(=O)NC2=O)C3=O)CC1] | 3-(4-(((1r,4r)-4- aminocyclohexyl)(3-(tetrahydro- 2H-pyran-2-yl)propyl)amino)-1- oxoisoindolin-2-yl)piperidine-2,6- dione |
| 344 | 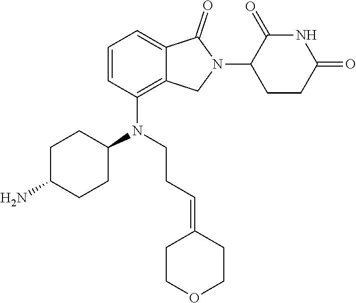 [SMILES: N[C@H]1CC[C@H](N(CCC=C2CCOCC2)c2cccc3c2CN(C2CCC(=O)NC2=O)C3=O)CC1] | 3-(4-(((1r,4r)-4- aminocyclohexyl)(3-(dihydro-2H- pyran-4(3H)- ylidene)propyl)amino)-1- oxoisoindolin-2-yl)piperidine-2,6- dione |
| 345 | 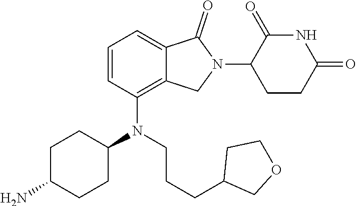 [SMILES: N[C@H]1CC[C@H](N(CCCC2CCOC2)c2cccc3c2CN(C2CCC(=O)NC2=O)C3=O)CC1] | 3-(4-(((1r,4r)-4- aminocyclohexyl)(3- (tetrahydrofuran-3- yl)propyl)amino)-1-oxoisoindolin- 2-yl)piperidine-2,6-dione |
| 346 | 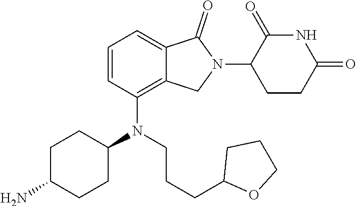 [SMILES: N[C@H]1CC[C@H](N(CCCC2CCCO2)c2cccc3c2CN(C2CCC(=O)NC2=O)C3=O)CC1] | 3-(4-(((1r,4r)-4- aminocyclohexyl)(3- (tetrahydrofuran-2- yl)propyl)amino)-1-oxoisoindolin- 2-yl)piperidine-2,6-dione |
| 347 | 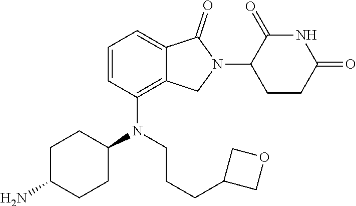 [SMILES: N[C@H]1CC[C@H](N(CCCC2COC2)c2cccc3c2CN(C2CCC(=O)NC2=O)C3=O)CC1] | 3-(4-(((1r,4r)-4- aminocyclohexyl)(3-(oxetan-3- yl)propyl)amino)-1-oxoisoindolin- 2-yl)piperidine-2,6-dione |
| 349 | 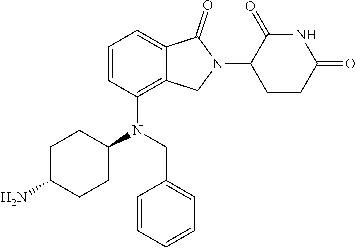 [SMILES: N[C@H]1CC[C@H](N(Cc2ccccc2)c2cccc3c2CN(C2CCC(=O)NC2=O)C3=O)CC1] | 3-(4-(((1r,4r)-4- aminocyclohexyl)(benzyl)amino)- 1-oxoisoindolin-2-yl)piperidine- 2,6-dione |
| 352 | 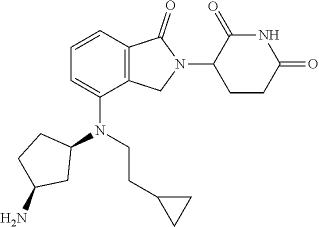 [SMILES: N[C@H]1CC[C@@H](N(CCC2CC2)c2cccc3c2CN(C2CCC(=O)NC2=O)C3=O)C1] | 3-(4-(((1R,3S)-3- aminocyclopentyl)(2- cyclopropylethyl)amino)-1- oxoisoindolin-2-yl)piperidine-2,6- dione |
| 353 | 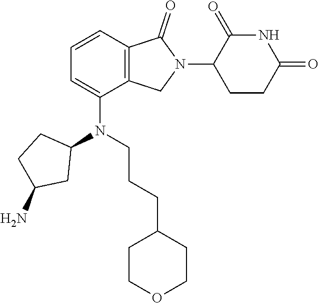 [SMILES: N[C@H]1CC[C@@H](N(CCCC2CCOCC2)c2cccc3c2CN(C2CCC(=O)NC2=O)C3=O)C1] | 3-(4-(((1R,3S)-3- aminocyclopentyl)(3-(tetrahydro- 2H-pyran-4-yl)propyl)amino)-1- oxoisoindolin-2-yl)piperidine-2,6- dione |
| 354 | 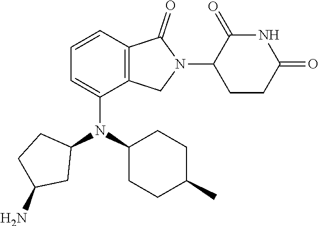 [SMILES: C[C@H]1CC[C@@H](N(c2cccc3c2CN(C2CCC(=O)NC2=O)C3=O)[C@@H]2CC[C@H](N)C2)CC1] | 3-(4-(((1R,3S)-3- aminocyclopentyl)((1s,4S)-4- methylcyclohexyl)amino)-1- oxoisoindolin-2-yl)piperidine-2,6- dione |
| 355 | 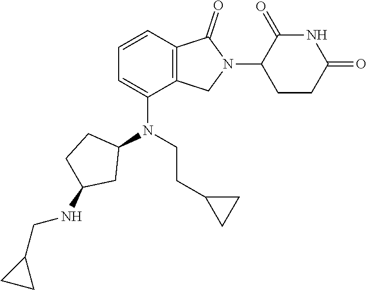 [SMILES: O=C1CCC(N2Cc3c(cccc3N(CCC3CC3)[C@@H]3CC[C@H](NCC4CC4)C3)C2=O)C(=O)N1] | 3-(4-((2-cyclopropylethyl)((1R,3S)- 3-((cyclopropylmethyl) amino)cyclopentyl) amino)-1-oxoisoindolin-2- yl)piperidine-2,6-dione |
| 356 | 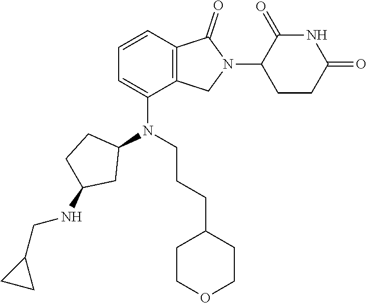 [SMILES: O=C1CCC(N2Cc3c(cccc3N(CCCC3CCOCC3)[C@@H]3CC[C@H](NCC4CC4)C3)C2=O)C(=O)N1] | 3-(4-(((1R,3S)-3-((cyclo- propylmethyl)amino)cyclopentyl) (3-(tetrahydro-2H-pyran-4- yl)propyl)amino)-1-oxoisoindolin- 2-yl)piperidine-2,6-dione |
| 358 | 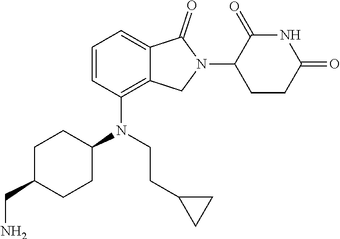 [SMILES: NC[C@H]1CC[C@@H](N(CCC2CC2)c2cccc3c2CN(C2CCC(=O)NC2=O)C3=O)CC1] | 3-(4-(((1s,4s)-4- (aminomethyl)cyclohexyl)(2- cyclopropylethyl)amino)-1- oxoisoindolin-2-yl)piperidine-2,6- dione |
| 359 | 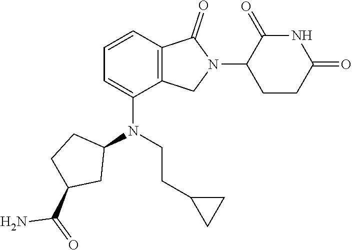 [SMILES: NC(=O)[C@H]1CC[C@@H](N(CCC2CC2)c2cccc3c2CN(C2CCC(=O)NC2=O)C3=O)C1] | (1S,3R)-3-((2-cyclopropylethyl)(2- (2,6-dioxopiperidin-3-yl)-1- oxoisoindolin-4- yl)amino)cyclopentanecarboxamide |
| 360 | 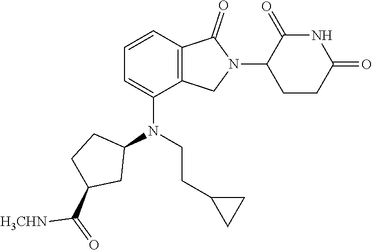 | (1S,3R)-3-((2-cyclopropylethyl)(2- (2,6-dioxopiperidin-3-yl)-1- oxoisoindolin-4-yl)amino)-N- methylcyclopentanecarboxamide |
| 361 | 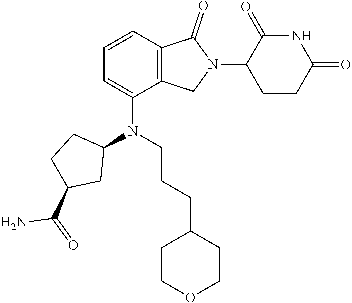 [SMILES: NC(=O)[C@H]1CC[C@@H](N(CCCC2CCOCC2)c2cccc3c2CN(C2CCC(=O)NC2=O)C3=O)C1] | (1S,3R)-3-((2-(2,6-dioxopiperidin- 3-yl)-1-oxoisoindolin-4-yl)(3- (tetrahydro-2H-pyran-4- yl)propyl)amino)cyclopentane- carboxamide |
| 362 | 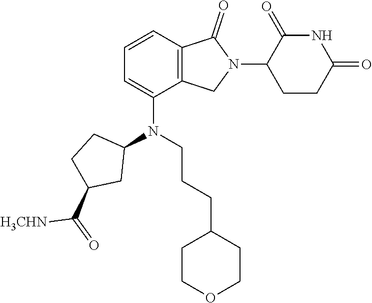 | (1S,3R)-3-((2-(2,6-dioxopiperidin- 3-yl)-1-oxoisoindolin-4-yl)(3- (tetrahydro-2H-pyran-4- yl)propyl)amino)-N- methylcyclopentanecarboxamide |
| 512 | 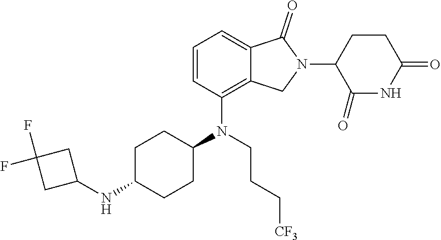 [SMILES: O=C1CCC(N2Cc3c(cccc3N(CCCC(F)(F)F)[C@H]3CC[C@H](NC4CC(F)(F)C4)CC3)C2=O)C(=O)N1] | 3-(4-(((1r,4r)-4-((3,3- difluorocyclobutyl)amino)cyclo- hexyl)(4,4,4-trifluorobutyl)amino)-1- oxoisoindolin-2-yl)piperidine-2,6- dione |
| 513 | 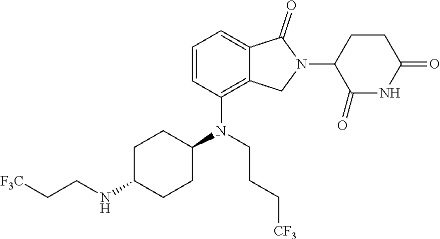 [SMILES: O=C1CCC(N2Cc3c(cccc3N(CCCC(F)(F)F)[C@H]3CC[C@H](NCCC(F)(F)F)CC3)C2=O)C(=O)N1] | 3-(1-oxo-4-((4,4,4- trifluorobutyl)((1r,4r)-4-((3,3,3- trifluoropropyl)amino)cyclohexyl) amino)isoindolin-2-yl)piperidine- 2,6-dione |
| 514 | 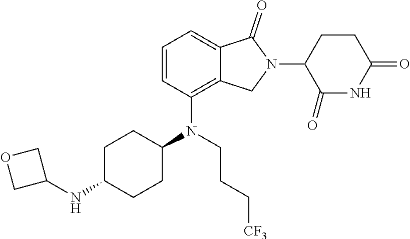 [SMILES: O=C1CCC(N2Cc3c(cccc3N(CCCC(F)(F)F)[C@H]3CC[C@H](NC4COC4)CC3)C2=O)C(=O)N1] | 3-(4-(((1r,4r)-4-(oxetan-3- ylamino)cyclohexyl)(4,4,4- trifluorobutyl)amino)-1- oxoisoindolin-2-yl)piperidine-2,6- dione |
| 515 | 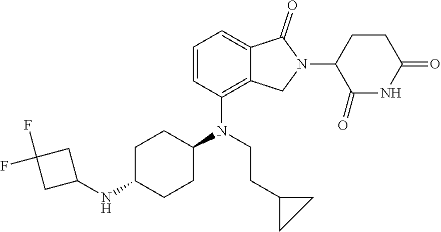 [SMILES: O=C1CCC(N2Cc3c(cccc3N(CCC3CC3)[C@H]3CC[C@H](NC4CC(F)(F)C4)CC3)C2=O)C(=O)N1] | 3-(4-((2-cyclopropylethyl)((1r,4r)- 4-((3,3-difluoro- cyclobutyl)amino)cyclohexyl) amino)-1-oxoisoindolin-2- yl)piperidine-2,6-dione |
| 515A | 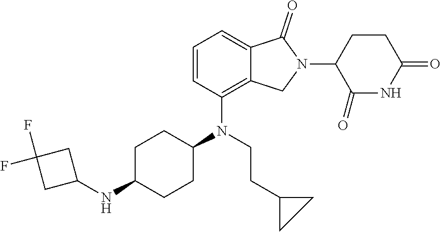 [SMILES: O=C1CCC(N2Cc3c(cccc3N(CCC3CC3)[C@H]3CC[C@@H](NC4CC(F)(F)C4)CC3)C2=O)C(=O)N1] | 3-(4-((2-cyclopropylethyl)((1s,4s)- 4-((3,3-difluoro- cyclobutyl)amino)cyclohexyl) amino)-1-oxoisoindolin-2- yl)piperidine-2,6-dione |
| 516 | 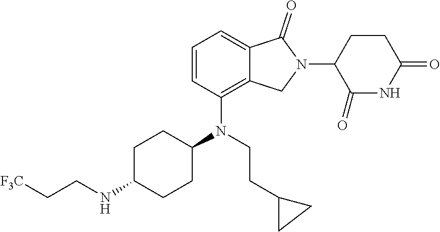 [SMILES: O=C1CCC(N2Cc3c(cccc3N(CCC3CC3)[C@H]3CC[C@H](NCCC(F)(F)F)CC3)C2=O)C(=O)N1] | 3-(4-((2-cyclopropylethyl)((1r,4r)- 4-((3,3,3- trifluoropropyl)amino)cyclohexyl) amino)-1-oxoisoindolin-2- yl)piperidine-2,6-dione |
| 517 | 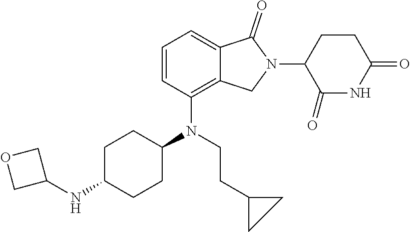 [SMILES: O=C1CCC(N2Cc3c(cccc3N(CCC3CC3)[C@H]3CC[C@H](NC4COC4)CC3)C2=O)C(=O)N1] | 3-(4-((2-cyclopropylethyl)((1r,4r)- 4-(oxetan-3- ylamino)cyclohexyl)amino)-1- oxoisoindolin-2-yl)piperidine-2,6- dione |
| 518 | 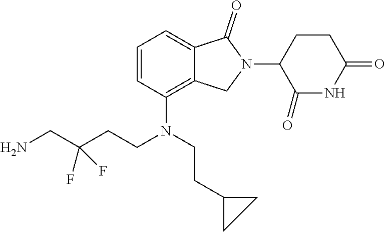 [SMILES: NCC(F)(F)CCN(CCC1CC1)c1cccc2c1CN(C1CCC(=O)NC1=O)C2=O] | 3-(4-((4-amino-3,3- difluorobutyl)(2- cyclopropylethyl)amino)-1- oxoisoindolin-2-yl)piperidine-2,6- dione |
| 519 | 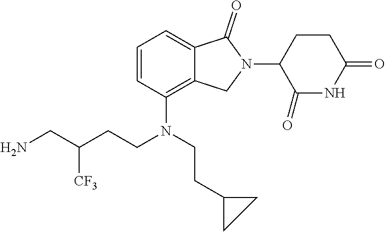 [SMILES: NCC(CCN(CCC1CC1)c1cccc2c1CN(C1CCC(=O)NC1=O)C2=O)C(F)(F)F] | 3-(4-((3-(aminomethyl)-4,4,4- trifluorobutyl)(2- cyclopropylethyl)amino)-1- oxoisoindolin-2-yl)piperidine-2,6- dione |
| 521 | 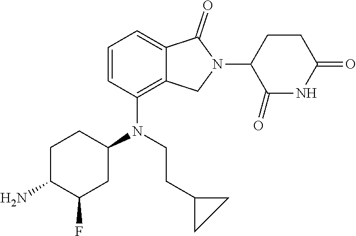 [SMILES: N[C@@H]1CC[C@@H](N(CCC2CC2)c2cccc3c2CN(C2CCC(=O)NC2=O)C3=O)C[C@H]1F] | 3-(4-(((1R,3R,4R)-4-amino-3- fluorocyclohexyl)(2- cyclopropylethyl)amino)-1- oxoisoindolin-2-yl)piperidine-2,6- dione |
| 522 | 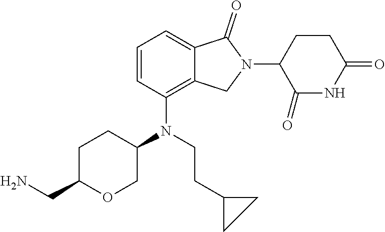 [SMILES: NC[C@H]1CC[C@@H](N(CCC2CC2)c2cccc3c2CN(C2CCC(=O)NC2=O)C3=O)CO1] | 3-(4-(((3R,6R)-6- (aminomethyl)tetrahydro-2H- pyran-3-yl)(2- cyclopropylethyl)amino)-1- oxoisoindolin-2-yl)piperidine-2,6- dione |
| 523 | 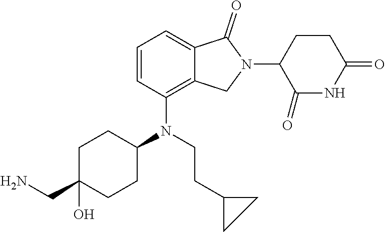 [SMILES: NC[C@]1(O)CC[C@H](N(CCC2CC2)c2cccc3c2CN(C2CCC(=O)NC2=O)C3=O)CC1] | 3-(4-(((1r,4r)-4-(aminomethyl)-4- hydroxycyclohexyl)(2- cyclopropylethyl)amino)-1- oxoisoindolin-2-yl)piperidine-2,6- dione |
| 530 | 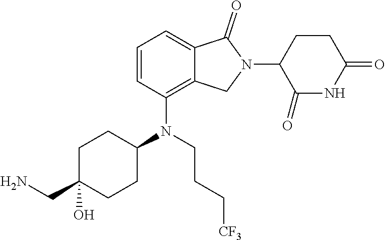 [SMILES: NC[C@]1(O)CC[C@H](N(CCCC(F)(F)F)c2cccc3c2CN(C2CCC(=O)NC2=O)C3=O)CC1] | 3-(4-(((1r,4r)-4-(aminomethyl)-4- hydroxycyclohexyl)(4,4,4- trifluorobutyl)amino)-1- oxoisoindolin-2-yl)piperidine-2,6- dione |
| 532 | 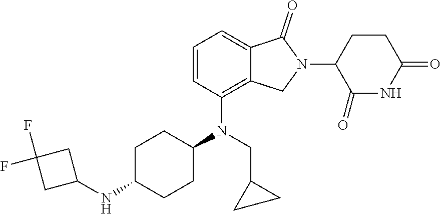 [SMILES: O=C1CCC(N2Cc3c(cccc3N(CC3CC3)[C@H]3CC[C@H](NC4CC(F)(F)C4)CC3)C2=O)C(=O)N1] | 3-(4-((cyclopropylmethyl)((1r,4r)- 4-((3,3-difluoro- cyclobutyl)amino)cyclohexyl) amino)-1-oxoisoindolin-2- yl)piperidine-2,6-dione |
| 533 | 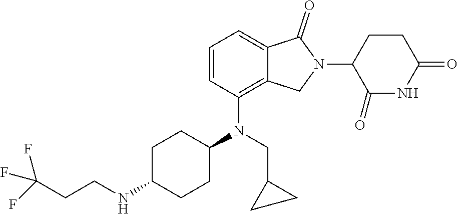 [SMILES: O=C1CCC(N2Cc3c(cccc3N(CC3CC3)[C@H]3CC[C@H](NCCC(F)(F)F)CC3)C2=O)C(=O)N1] | 3-(4-((cyclopropylmethyl)((1r,4r)- 4-((3,3,3- trifluoropropyl)amino)cyclohexyl) amino)-1-oxoisoindolin-2- yl)piperidine-2,6-dione |
| 534 | 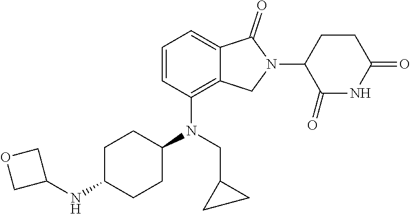 [SMILES: O=C1CCC(N2Cc3c(cccc3N(CC3CC3)[C@H]3CC[C@H](NC4COC4)CC3)C2=O)C(=O)N1] | 3-(4-((cyclopropylmethyl)((1r,4r)- 4-(oxetan-3- ylamino)cyclohexyl)amino)-1- oxoisoindolin-2-yl)piperidine-2,6- dione |
| 535 | 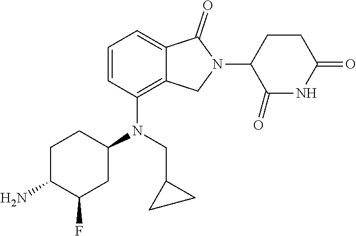 [SMILES: N[C@@H]1CC[C@@H](N(CC2CC2)c2cccc3c2CN(C2CCC(=O)NC2=O)C3=O)C[C@H]1F] | 3-(4-(((1R,3R,4R)-4-amino-3- fluorocyclohexyl)(cyclopropylmethyl) amino)-1-oxoisoindolin-2- yl)piperidine-2,6-dione |
| 536 | 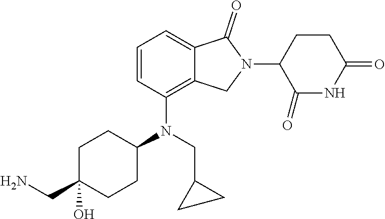 [SMILES: NC[C@]1(O)CC[C@H](N(CC2CC2)c2cccc3c2CN(C2CCC(=O)NC2=O)C3=O)CC1] | 3-(4-(((1r,4r)-4-(aminomethyl)-4- hydroxycyclohexyl)(cyclopropyl- methyl)amino)-1-oxoisoindolin-2- yl)piperidine-2,6-dione |
| 539 | 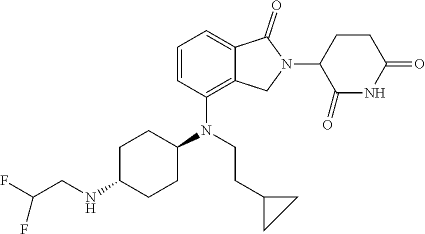 [SMILES: O=C1CCC(N2Cc3c(cccc3N(CCC3CC3)[C@H]3CC[C@H](NCC(F)F)CC3)C2=O)C(=O)N1] | 3-(4-((2-cyclopropylethyl)((1r,4r)- 4-((2,2- difluoroethyl)amino)cyclohexyl) amino)-1-oxoisoindolin-2- yl)piperidine-2,6-dione |
| 539A | 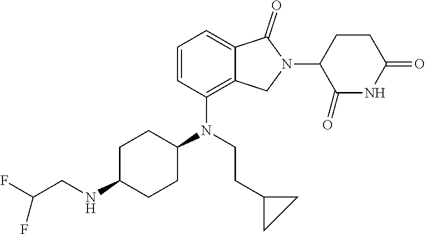 [SMILES: O=C1CCC(N2Cc3c(cccc3N(CCC3CC3)[C@H]3CC[C@@H](NCC(F)F)CC3)C2=O)C(=O)N1] | 3-(4-((2-cyclopropylethyl)((1s,4s)- 4-((2,2- difluoroethyl)amino)cyclohexyl) amino)-1-oxoisoindolin-2- yl)piperidine-2,6-dione |
| 540 | 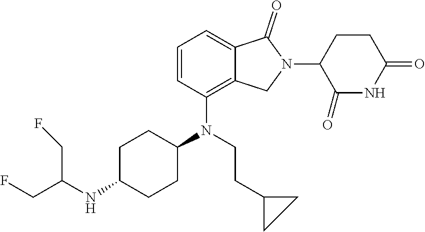 [SMILES: O=C1CCC(N2Cc3c(cccc3N(CCC3CC3)[C@H]3CC[C@H](NC(CF)CF)CC3)C2=O)C(=O)N1] | 3-(4-((2-cyclopropylethyl)((1r,4r)- 4-((1,3-difluoropropan-2- yl)amino)cyclohexyl)amino)-1- oxoisoindolin-2-yl)piperidine-2,6- dione |
| 541 | 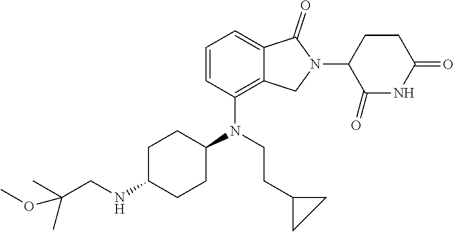 [SMILES: COC(C)(C)CN[C@H]1CC[C@H](N(CCC2CC2)c2cccc3c2CN(C2CCC(=O)NC2=O)C3=O)CC1] | 3-(4-((2-cyclopropylethyl)((1r,4r)- 4-((2-methoxy-2- methylpropyl)amino)cyclohexyl) amino)-1-oxoisoindolin-2- yl)piperidine-2,6-dione |
| 541A | 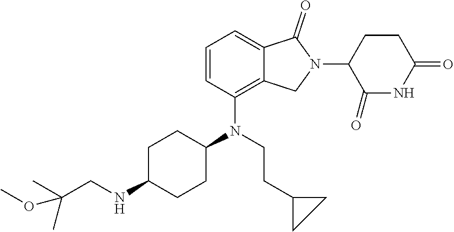 [SMILES: COC(C)(C)CN[C@H]1CC[C@@H](N(CCC2CC2)c2cccc3c2CN(C2CCC(=O)NC2=O)C3=O)CC1] | 3-(4-((2-cyclopropylethyl)((1s,4s)- 4-((2-methoxy-2- methylpropyl)amino)cyclohexyl) amino)-1-oxoisoindolin-2- yl)piperidine-2,6-dione |
| 542 | 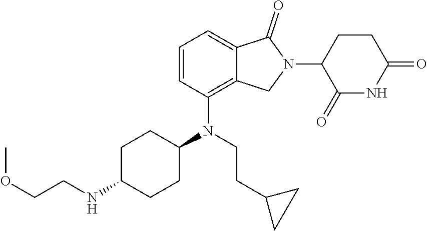 [SMILES: COCCN[C@H]1CC[C@H](N(CCC2CC2)c2cccc3c2CN(C2CCC(=O)NC2=O)C3=O)CC1] | 3-(4-((2-cyclopropylethyl)((1r,4r)- 4-((2- methoxyethyl)amino)cyclohexyl) amino)-1-oxoisoindolin-2- yl)piperidine-2,6-dione |
| 543 | 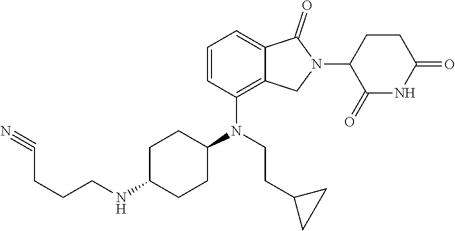 [SMILES: N#CCCCN[C@H]1CC[C@H](N(CCC2CC2)c2cccc3c2CN(C2CCC(=O)NC2=O)C3=O)CC1] | 4-(((1r,4r)-4-((2- cyclopropylethyl)(2-(2,6- dioxopiperidin-3-yl)-1- oxoisoindolin-4- yl)amino)cyclohexyl)amino)butane nitrile |
| 546 | 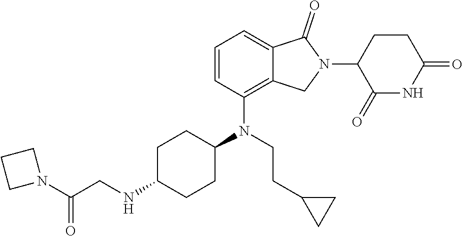 [SMILES: O=C1CCC(N2Cc3c(cccc3N(CCC3CC3)[C@H]3CC[C@H](NCC(=O)N4CCC4)CC3)C2=O)C(=O)N1] | 3-(4-(((1r,4r)-4-((2-(azetidin-1-yl)- 2-oxoethyl)amino)cyclohexyl)(2- cyclopropylethyl)amino)-1- oxoisoindolin-2-yl)piperidine-2,6- dione |
| 547 | 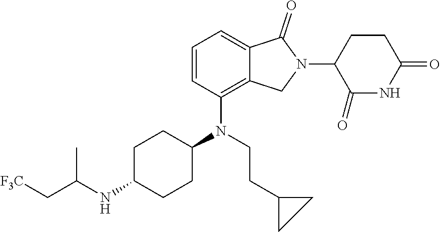 [SMILES: CC(CC(F)(F)F)N[C@H]1CC[C@H](N(CCC2CC2)c2cccc3c2CN(C2CCC(=O)NC2=O)C3=O)CC1] | 3-(4-((2-cyclopropylethyl)((1r,4r)- 4-((4,4,4-trifluorobutan-2- yl)amino)cyclohexyl)amino)-1- oxoisoindolin-2-yl)piperidine-2,6- dione |
| 548 | 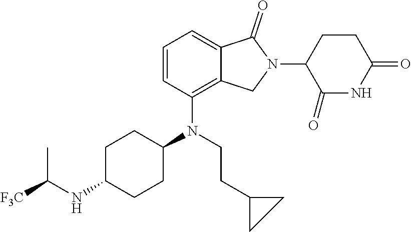 [SMILES: C[C@@H](N[C@H]1CC[C@H](N(CCC2CC2)c2cccc3c2CN(C2CCC(=O)NC2=O)C3=O)CC1)C(F)(F)F] | 3-(4-((2-cyclopropylethyl)((1R,4r)- 4-(((R)-1,1,1-trifluoropropan-2- yl)amino)cyclohexyl)amino)-1- oxoisoindolin-2-yl)piperidine-2,6- dione |
| 548A | 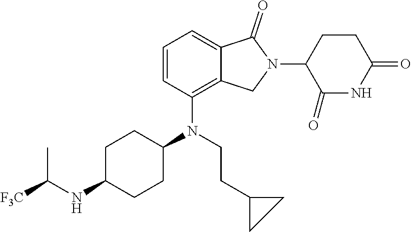 [SMILES: C[C@@H](N[C@H]1CC[C@@H](N(CCC2CC2)c2cccc3c2CN(C2CCC(=O)NC2=O)C3=O)CC1)C(F)(F)F] | 3-(4-((2-cyclopropylethyl)((1S,4s)- 4-(((R)-1,1,1-trifluoropropan-2- yl)amino)cyclohexyl)amino)-1- oxoisoindolin-2-yl)piperidine-2,6- dione |
| 549 | 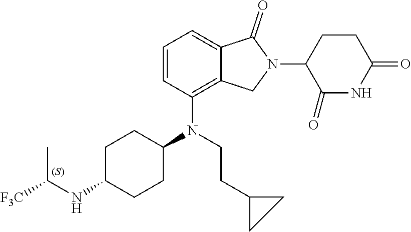 | 3-(4-((2-cyclopropylethyl)((1S,4r)- 4-(((S)-1,1,1-trifluoropropan-2- yl)amino)cyclohexyl)amino)-1- oxoisoindolin-2-yl)piperidine-2,6- dione |
| 550 | 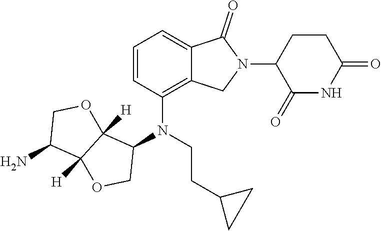 [SMILES: N[C@H]1CO[C@H]2[C@@H]1OC[C@@H]2N(CCC1CC1)c1cccc2c1CN(C1CCC(=O)NC1=O)C2=O] | 3-(4-(((3S,3aR,6S,6aR)-6- aminohexahydrofuro[3,2-b]furan-3- yl)(2-cyclopropylethyl)amino)-1- oxoisoindolin-2-yl)piperidine-2,6- dione |
| 552 | 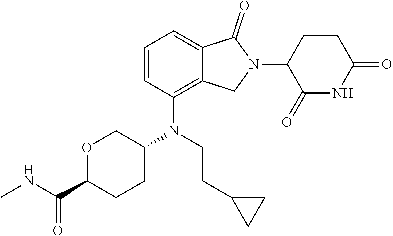 [SMILES: CNC(=O)[C@@H]1CC[C@@H](N(CCC2CC2)c2cccc3c2CN(C2CCC(=O)NC2=O)C3=O)CO1] | (2S,5R)-5-((2-cyclopropylethyl)(2- (2,6-dioxopiperidin-3-yl)-1- oxoisoindolin-4-yl)amino)-N- methyltetrahydro-2H-pyran-2- carboxamide |
| 554 | 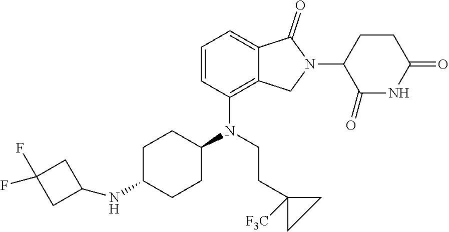 [SMILES: O=C1CCC(N2Cc3c(cccc3N(CCC3(C(F)(F)F)CC3)[C@H]3CC[C@H](NC4CC(F)(F)C4)CC3)C2=O)C(=O)N1] | 3-(4-(((1r,4r)-4-((3,3- difluorocyclobutyl)amino) cyclohexyl)(2-(1- (trifluoromethyl)cyclopropyl)ethyl) amino)-1-oxoisoindolin-2- yl)piperidine-2,6-dione |
| 554A | 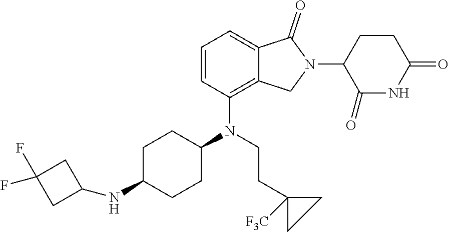 [SMILES: O=C1CCC(N2Cc3c(cccc3N(CCC3(C(F)(F)F)CC3)[C@H]3CC[C@@H](NC4CC(F)(F)C4)CC3)C2=O)C(=O)N1] | 3-(4-(((1s,4s)-4-((3,3- difluorocyclobutyl)amino) cyclohexyl)(2-(1- (trifluoromethyl)cyclopropyl)ethyl) amino)-1-oxoisoindolin-2- yl)piperidine-2,6-dione |
| 557 | 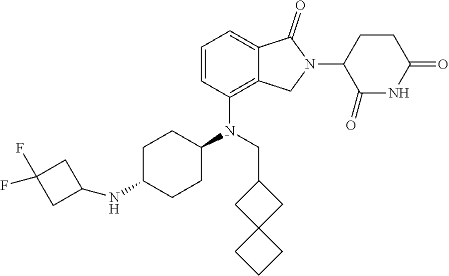 [SMILES: O=C1CCC(N2Cc3c(cccc3N(CC3CC4(CCC4)C3)[C@H]3CC[C@H](NC4CC(F)(F)C4)CC3)C2=O)C(=O)N1] | 3-(4-(((1r,4r)-4-((3,3- difluorocyclobutyl)amino) cyclohexyl)(spiro[3.3]heptan-2- ylmethyl)amino)-1-oxoisoindolin- 2-yl)piperidine-2,6-dione |
| 558-2 | 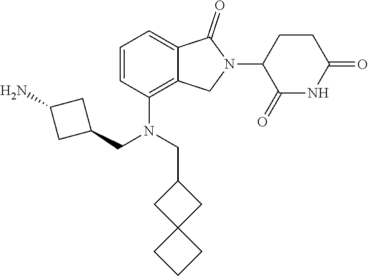 [SMILES: N[C@H]1C[C@H](CN(CC2CC3(CCC3)C2)c2cccc3c2CN(C2CCC(=O)NC2=O)C3=O)C1] | 3-(4-((((1r,3r)-3- aminocyclobutyl)methyl)(spiro[3.3] heptan-2-ylmethyl)amino)-1- oxoisoindolin-2-yl)piperidine-2,6- dione |
| 561 | 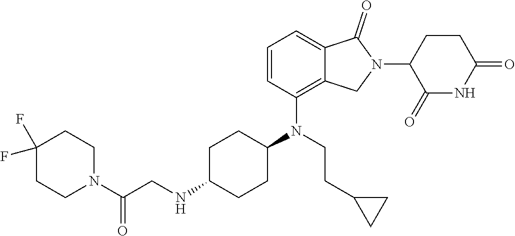 [SMILES: O=C1CCC(N2Cc3c(cccc3N(CCC3CC3)[C@H]3CC[C@H](NCC(=O)N4CCC(F)(F)CC4)CC3)C2=O)C(=O)N1] | 3-(4-((2-cyclopropylethyl)((1r,4r)- 4-((2-(4,4-difluoropiperidin-1-yl)-2- oxoethyl)amino)cyclohexyl)amino)- 1-oxoisoindolin-2-yl)piperidine- 2,6-dione |
| 562 | 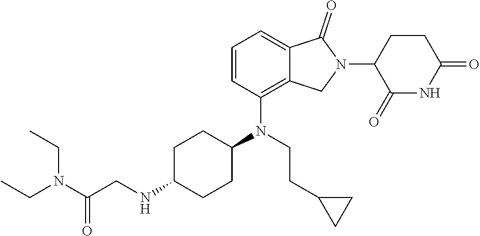 [SMILES: CCN(CC)C(=O)CN[C@H]1CC[C@H](N(CCC2CC2)c2cccc3c2CN(C2CCC(=O)NC2=O)C3=O)CC1] | 2-(((1r,4r)-4-((2- cyclopropylethyl)(2-(2,6- dioxopiperidin-3-yl)-1- oxoisoindolin-4- yl)amino)cyclohexyl)amino)-N,N- diethylacetamide |
| 573 | 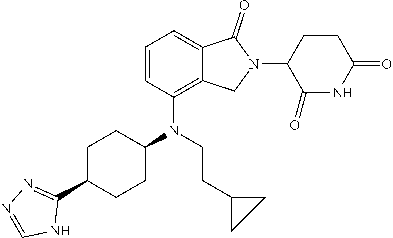 [SMILES: O=C1CCC(N2Cc3c(cccc3N(CCC3CC3)[C@H]3CC[C@@H](c4nnc[nH]4)CC3)C2=O)C(=O)N1] | 3-(4-(((1s,4s)-4-(4H-1,2,4-triazol-3- yl)cyclohexyl)(2- cyclopropylethyl)amino)-1- oxoisoindolin-2-yl)piperidine-2,6- dione |
| 574 | 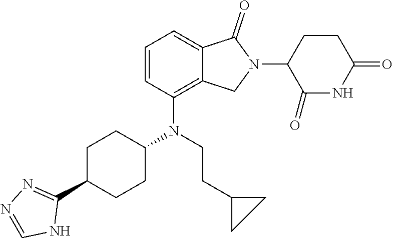 [SMILES: O=C1CCC(N2Cc3c(cccc3N(CCC3CC3)[C@H]3CC[C@H](c4nnc[nH]4)CC3)C2=O)C(=O)N1] | 3-(4-(((1r,4r)-4-(4H-1,2,4-triazol-3- yl)cyclohexyl)(2- cyclopropylethyl)amino)-1- oxoisoindolin-2-yl)piperidine-2,6- dione |
| 575 | 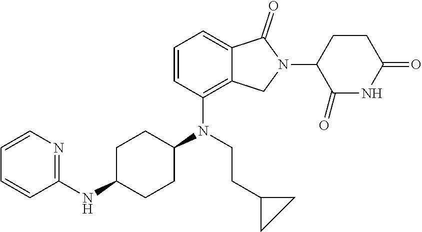 [SMILES: O=C1CCC(N2Cc3c(cccc3N(CCC3CC3)[C@H]3CC[C@@H](Nc4ccccn4)CC3)C2=O)C(=O)N1] | 3-(4-((2-cyclopropylethyl)((1s,4s)- 4-(pyridin-2- ylamino)cyclohexyl)amino)-1- oxoisoindolin-2-yl)piperidine-2,6- dione |
| 576 | 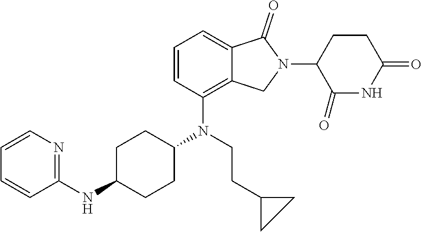 [SMILES: O=C1CCC(N2Cc3c(cccc3N(CCC3CC3)[C@H]3CC[C@H](Nc4ccccn4)CC3)C2=O)C(=O)N1] | 3-(4-((2-cyclopropylethyl)((1r,4r)- 4-(pyridin-2- ylamino)cyclohexyl)amino)-1- oxoisoindolin-2-yl)piperidine-2,6- dione |
| 577 | 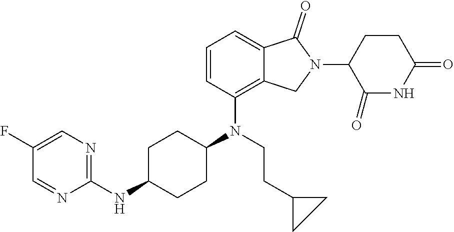 [SMILES: O=C1CCC(N2Cc3c(cccc3N(CCC3CC3)[C@H]3CC[C@@H](Nc4ncc(F)cn4)CC3)C2=O)C(=O)N1] | 3-(4-((2-cyclopropylethyl)((1s,4s)- 4-((5-fluoropyrimidin-2- yl)amino)cyclohexyl)amino)-1- oxoisoindolin-2-yl)piperidine-2,6- dione |
| 578 | 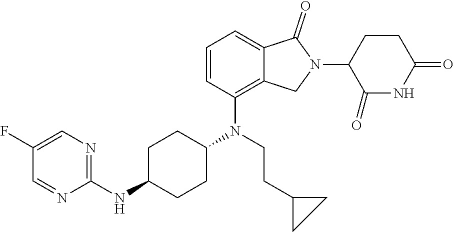 [SMILES: O=C1CCC(N2Cc3c(cccc3N(CCC3CC3)[C@H]3CC[C@H](Nc4ncc(F)cn4)CC3)C2=O)C(=O)N1] | 3-(4-((2-cyclopropylethyl)((1r,4r)- 4-((5-fluoropyrimidin-2- yl)amino)cyclohexyl)amino)-1- oxoisoindolin-2-yl)piperidine-2,6- dione |
| 580 | 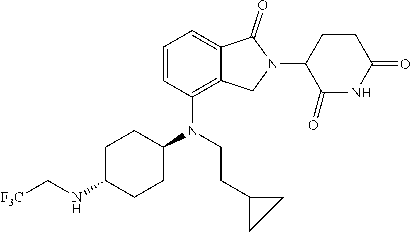 [SMILES: O=C1CCC(N2Cc3c(cccc3N(CCC3CC3)[C@H]3CC[C@H](NCC(F)(F)F)CC3)C2=O)C(=O)N1] | 3-(4-((2-cyclopropylethyl)((1r,4r)- 4-((2,2,2- trifluoroethyl)amino)cyclohexyl) amino)-1-oxoisoindolin-2- yl)piperidine-2,6-dione |
| 581 | 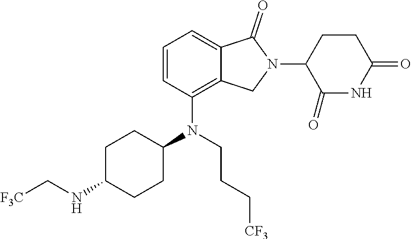 [SMILES: O=C1CCC(N2Cc3c(cccc3N(CCCC(F)(F)F)[C@H]3CC[C@H](NCC(F)(F)F)CC3)C2=O)C(=O)N1] | 3-(1-oxo-4-((4,4,4- trifluorobutyl)((1r,4r)-4-((2,2,2- trifluoroethyl)amino)cyclohexyl) amino)isoindolin-2-yl)piperidine-2,6- dione |
| 582 | 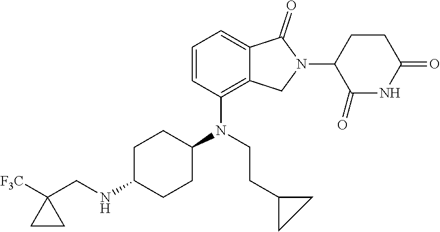 [SMILES: O=C1CCC(N2Cc3c(cccc3N(CCC3CC3)[C@H]3CC[C@H](NCC4(C(F)(F)F)CC4)CC3)C2=O)C(=O)N1] | 3-(4-((2-cyclopropylethyl)((1r,4r)- 4-(((1- (trifluoromethyl)cyclopropyl)methyl) amino)cyclohexyl)amino)-1- oxoisoindolin-2-yl)piperidine-2,6- dione |
| 583 | 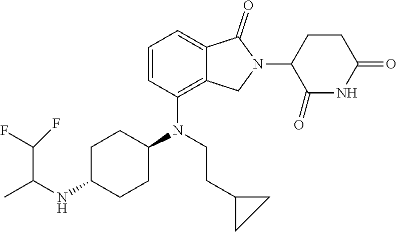 [SMILES: CC(N[C@H]1CC[C@H](N(CCC2CC2)c2cccc3c2CN(C2CCC(=O)NC2=O)C3=O)CC1)C(F)F] | 3-(4-((2-cyclopropylethyl)((1r,4r)- 4-((1,1-difluoropropan-2- yl)amino)cyclohexyl)amino)-1- oxoisoindolin-2-yl)piperidine-2,6- dione |
| 603 | 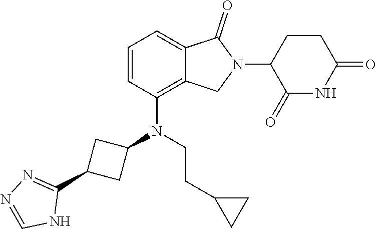 [SMILES: O=C1CCC(N2Cc3c(cccc3N(CCC3CC3)[C@H]3C[C@@H](c4nnc[nH]4)C3)C2=O)C(=O)N1] | 3-(4-(((1s,3s)-3-(4H-1,2,4-triazol-3- yl)cyclobutyl)(2- cyclopropylethyl)amino)-1- oxoisoindolin-2-yl)piperidine-2,6- dione |
| 604 | 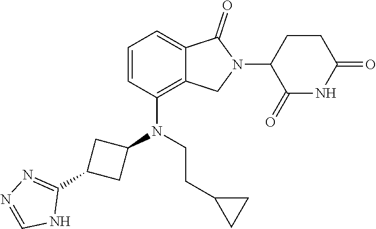 [SMILES: O=C1CCC(N2Cc3c(cccc3N(CCC3CC3)[C@H]3C[C@H](c4nnc[nH]4)C3)C2=O)C(=O)N1] | 3-(4-(((1r,3r)-3-(4H-1,2,4-triazol-3- yl)cyclobutyl)(2- cyclopropylethyl)amino)-1- oxoisoindolin-2-yl)piperidine-2,6- dione |
| 611 | 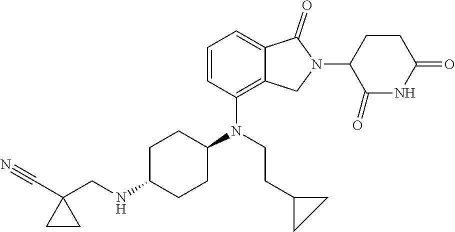 [SMILES: N#CC1(CN[C@H]2CC[C@H](N(CCC3CC3)c3cccc4c3CN(C3CCC(=O)NC3=O)C4=O)CC2)CC1] | 1-((((1r,4r)-4-((2- cyclopropylethyl)(2-(2,6- dioxopiperidin-3-yl)-1- oxoisoindolin-4- yl)amino)cyclohexyl)amino)methyl) cyclopropanecarbonitrile |
| 612 | 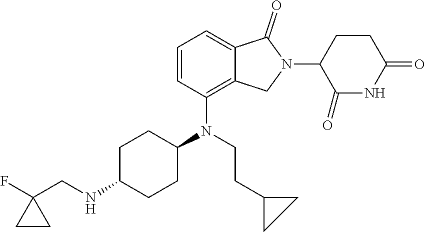 [SMILES: O=C1CCC(N2Cc3c(cccc3N(CCC3CC3)[C@H]3CC[C@H](NCC4(F)CC4)CC3)C2=O)C(=O)N1] | 3-(4-((2-cyclopropylethyl)((1r,4r)- 4-(((1- fluorocyclopropyl)methyl)amino) cyclohexyl)amino)-1-oxoisoindolin-2- yl)piperidine-2,6-dione |
| 612A | 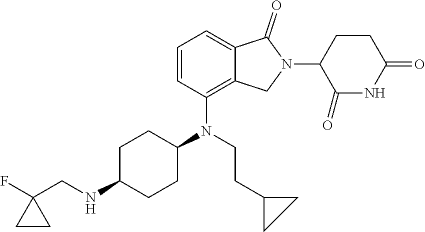 [SMILES: O=C1CCC(N2Cc3c(cccc3N(CCC3CC3)[C@H]3CC[C@@H](NCC4(F)CC4)CC3)C2=O)C(=O)N1] | 3-(4-((2-cyclopropylethyl)((1s,4s)- 4-(((1- fluorocyclopropyl)methyl)amino) cyclohexyl)amino)-1-oxoisoindolin-2- yl)piperidine-2,6-dione |
| 613 | 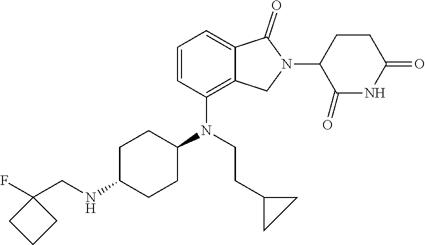 [SMILES: O=C1CCC(N2Cc3c(cccc3N(CCC3CC3)[C@H]3CC[C@H](NCC4(F)CCC4)CC3)C2=O)C(=O)N1] | 3-(4-((2-cyclopropylethyl)((1r,4r)- 4-(((1- fluorocyclobutyl)methyl)amino) cyclohexyl)amino)-1-oxoisoindolin-2- yl)piperidine-2,6-dione |
| 613A | 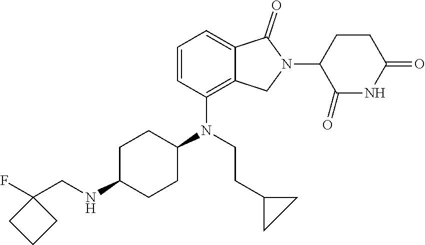 [SMILES: O=C1CCC(N2Cc3c(cccc3N(CCC3CC3)[C@H]3CC[C@@H](NCC4(F)CCC4)CC3)C2=O)C(=O)N1] | 3-(4-((2-cyclopropylethyl)((1s,4s)- 4-(((1- fluorocyclobutyl)methyl)amino) cyclohexyl)amino)-1-oxoisoindolin-2- yl)piperidine-2,6-dione |
| 614 | 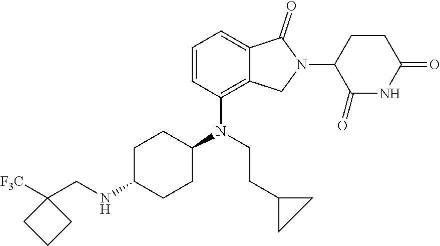 [SMILES: O=C1CCC(N2Cc3c(cccc3N(CCC3CC3)[C@H]3CC[C@H](NCC4(C(F)(F)F)CCC4)CC3)C2=O)C(=O)N1] | 3-(4-((2-cyclopropylethyl)((1r,4r)- 4-(((1- (trifluoromethyl)cyclobutyl)methyl) amino)cyclohexyl)amino)-1- oxoisoindolin-2-yl)piperidine-2,6- dione |
| 614A | 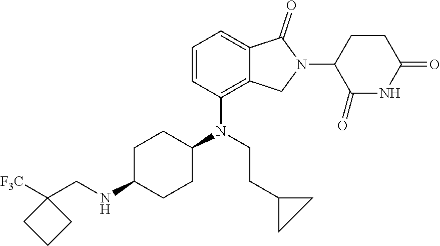 [SMILES: O=C1CCC(N2Cc3c(cccc3N(CCC3CC3)[C@H]3CC[C@@H](NCC4(C(F)(F)F)CCC4)CC3)C2=O)C(=O)N1] | 3-(4-((2-cyclopropylethyl)((1s,4s)- 4-(((1- (trifluoromethyl)cyclobutyl)methyl) amino)cyclohexyl)amino)-1- oxoisoindolin-2-yl)piperidine-2,6- dione |
| 615 | 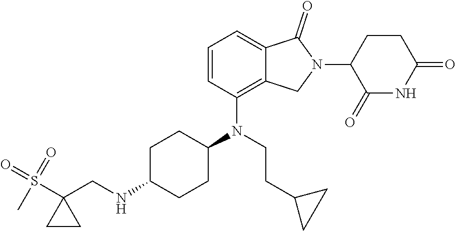 [SMILES: CS(=O)(=O)C1(CN[C@H]2CC[C@H](N(CCC3CC3)c3cccc4c3CN(C3CCC(=O)NC3=O)C4=O)CC2)CC1] | 3-(4-((2-cyclopropylethyl)((1r,4r)- 4-(((1- (methylsulfonyl)cyclopropyl)methyl) amino)cyclohexyl)amino)-1- oxoisoindolin-2-yl)piperidine-2,6- dione |
| 615A | 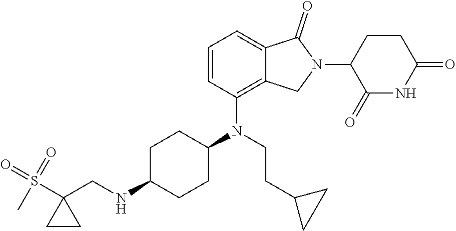 [SMILES: CS(=O)(=O)C1(CN[C@H]2CC[C@@H](N(CCC3CC3)c3cccc4c3CN(C3CCC(=O)NC3=O)C4=O)CC2)CC1] | 3-(4-((2-cyclopropylethyl)((1s,4s)- 4-(((1- (methylsulfonyl)cyclopropyl)methyl) amino)cyclohexyl)amino)-1- oxoisoindolin-2-yl)piperidine-2,6- dione |
| 616 | 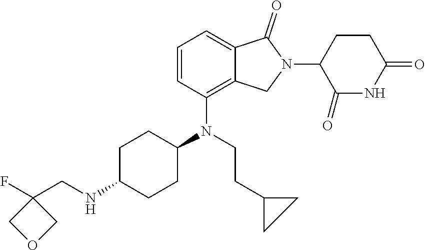 [SMILES: O=C1CCC(N2Cc3c(cccc3N(CCC3CC3)[C@H]3CC[C@H](NCC4(F)COC4)CC3)C2=O)C(=O)N1] | 3-(4-((2-cyclopropylethyl)((1r,4r)- 4-(((3-fluorooxetan-3- yl)methyl)amino)cyclohexyl)amino)- 1-oxoisoindolin-2-yl)piperidine- 2,6-dione |
| 616A | 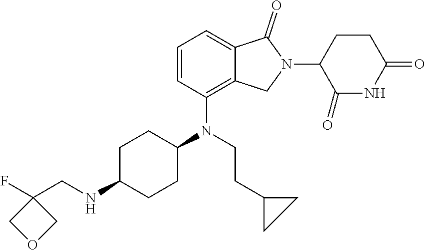 [SMILES: O=C1CCC(N2Cc3c(cccc3N(CCC3CC3)[C@H]3CC[C@@H](NCC4(F)COC4)CC3)C2=O)C(=O)N1] | 3-(4-((2-cyclopropylethyl)((1s,4s)- 4-(((3-fluorooxetan-3- yl)methyl)amino)cyclohexyl)amino)- 1-oxoisoindolin-2-yl)piperidine- 2,6-dione |
| 619 | 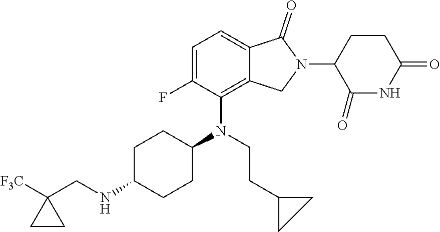 [SMILES: O=C1CCC(N2Cc3c(ccc(F)c3N(CCC3CC3)[C@H]3CC[C@H](NCC4(C(F)(F)F)CC4)CC3)C2=O)C(=O)N1] | 3-(4-((2-cyclopropylethyl)((1r,4r)- 4-(((1- (trifluoromethyl)cyclopropyl)methyl) amino)cyclohexyl)amino)-5- fluoro-1-oxoisoindolin-2- yl)piperidine-2,6-dione |
| 627 | 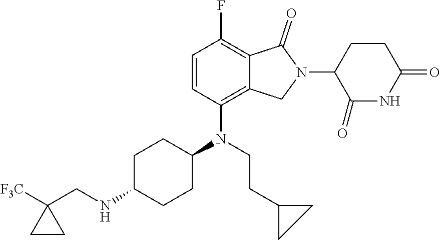 [SMILES: O=C1CCC(N2Cc3c(N(CCC4CC4)[C@H]4CC[C@H](NCC5(C(F)(F)F)CC5)CC4)ccc(F)c3C2=O)C(=O)N1] | 3-(4-((2-cyclopropylethyl)((1r,4r)- 4-(((1- (trifluoromethyl)cyclopropyl)methyl) amino)cyclohexyl)amino)-7- fluoro-1-oxoisoindolin-2- yl)piperidine-2,6-dione |
| 627A | 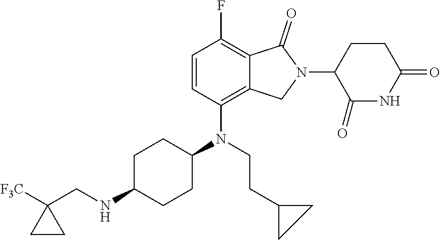 [SMILES: O=C1CCC(N2Cc3c(N(CCC4CC4)[C@H]4CC[C@@H](NCC5(C(F)(F)F)CC5)CC4)ccc(F)c3C2=O)C(=O)N1] | 3-(4-((2-cyclopropylethyl)((1s,4s)- 4-(((1- (trifluoromethyl)cyclopropyl)methyl) amino)cyclohexyl)amino)-7- fluoro-1-oxoisoindolin-2- yl)piperidine-2,6-dione |
| 628 | 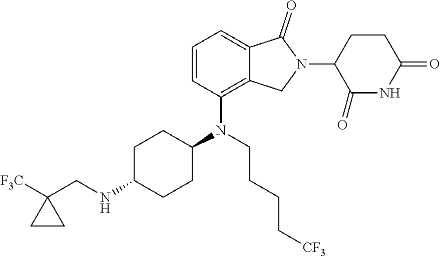 [SMILES: O=C1CCC(N2Cc3c(cccc3N(CCCCC(F)(F)F)[C@H]3CC[C@H](NCC4(C(F)(F)F)CC4)CC3)C2=O)C(=O)N1] | 3-(1-oxo-4-(((1r,4r)-4-(((1- (trifluoromethyl)cyclopropyl)methyl) amino)cyclohexyl)(5,5,5- trifluoropentyl)amino)isoindolin-2- yl)piperidine-2,6-dione |
| 628A | 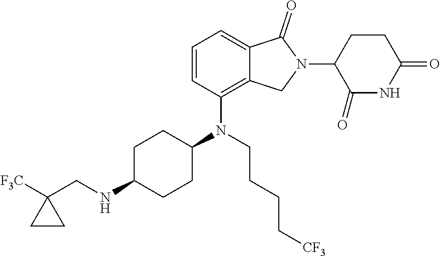 [SMILES: O=C1CCC(N2Cc3c(cccc3N(CCCCC(F)(F)F)[C@H]3CC[C@@H](NCC4(C(F)(F)F)CC4)CC3)C2=O)C(=O)N1] | 3-(1-oxo-4-(((1s,4s)-4-(((1- (trifluoromethyl)cyclopropyl)methyl) amino)cyclohexyl)(5,5,5- trifluoropentyl)amino)isoindolin-2- yl)piperidine-2,6-dione |
| 629 | 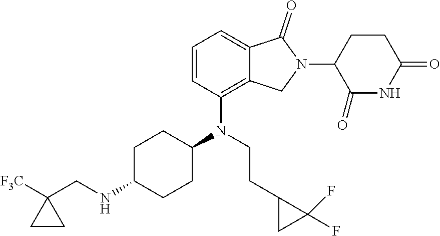 [SMILES: O=C1CCC(N2Cc3c(cccc3N(CCC3CC3(F)F)[C@H]3CC[C@H](NCC4(C(F)(F)F)CC4)CC3)C2=O)C(=O)N1] | 3-(4-((2-(2,2- difluorocyclopropyl)ethyl)((1r,4r)- 4-(((1- (trifluoromethyl)cyclopropyl)methyl) amino)cyclohexyl)amino)-1- oxoisoindolin-2-yl)piperidine-2,6- dione |
| 629A | 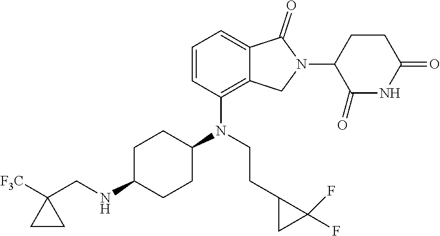 [SMILES: O=C1CCC(N2Cc3c(cccc3N(CCC3CC3(F)F)[C@H]3CC[C@@H](NCC4(C(F)(F)F)CC4)CC3)C2=O)C(=O)N1] | 3-(4-((2-(2,2- difluorocyclopropyl)ethyl)((1s,4s)- 4-(((1- (trifluoromethyl)cyclopropyl)methyl) amino)cyclohexyl)amino)-1- oxoisoindolin-2-yl)piperidine-2,6- dione |
| 630 | 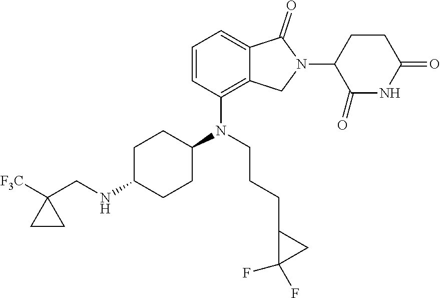 [SMILES: O=C1CCC(N2Cc3c(cccc3N(CCCC3CC3(F)F)[C@H]3CC[C@H](NCC4(C(F)(F)F)CC4)CC3)C2=O)C(=O)N1] | 3-(4-((3-(2,2- difluorocyclopropyl)propyl)((1r,4r)- 4-(((1- (trifluoromethyl)cyclopropyl)methyl) amino)cyclohexyl)amino)-1- oxoisoindolin-2-yl)piperidine-2,6- dione |
| 630A | 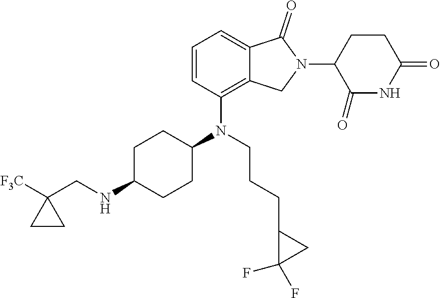 [SMILES: O=C1CCC(N2Cc3c(cccc3N(CCCC3CC3(F)F)[C@H]3CC[C@@H](NCC4(C(F)(F)F)CC4)CC3)C2=O)C(=O)N1] | 3-(4-((3-(2,2- difluorocyclopropyl)propyl)((1s,4s)- 4-(((1- (trifluoromethyl)cyclopropyl)methyl) amino)cyclohexyl)amino)-1- oxoisoindolin-2-yl)piperidine-2,6- dione |
| 634 | 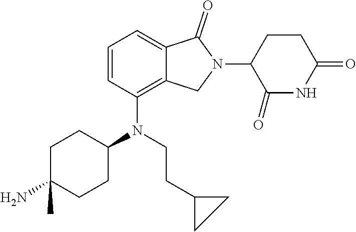 [SMILES: C[C@]1(N)CC[C@H](N(CCC2CC2)c2cccc3c2CN(C2CCC(=O)NC2=O)C3=O)CC1] | 3-(4-(((1r,4r)-4-amino-4- methylcyclohexyl)(2- cyclopropylethyl)amino)-1- oxoisoindolin-2-yl)piperidine-2,6- dione |
| 635 | 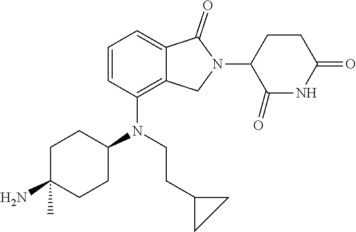 [SMILES: C[C@]1(N)CC[C@@H](N(CCC2CC2)c2cccc3c2CN(C2CCC(=O)NC2=O)C3=O)CC1] | 3-(4-(((1s,4s)-4-amino-4- methylcyclohexyl)(2- cyclopropylethyl)amino)-1- oxoisoindolin-2-yl)piperidine-2,6- dione |
| 636 | 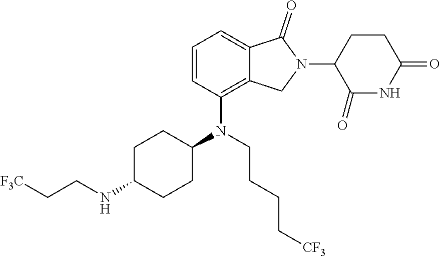 [SMILES: O=C1CCC(N2Cc3c(cccc3N(CCCCC(F)(F)F)[C@H]3CC[C@H](NCCC(F)(F)F)CC3)C2=O)C(=O)N1] | 3-(1-oxo-4-((5,5,5- trifluoropentyl)((1r,4r)-4-((3,3,3- trifluoropropyl)amino)cyclohexyl) amino)isoindolin-2-yl)piperidine- 2,6-dione |
| 636A | 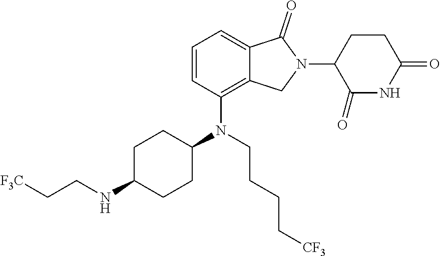 [SMILES: O=C1CCC(N2Cc3c(cccc3N(CCCCC(F)(F)F)[C@H]3CC[C@@H](NCCC(F)(F)F)CC3)C2=O)C(=O)N1] | 3-(1-oxo-4-((5,5,5- trifluoropentyl)((1s,4s)-4-((3,3,3- trifluoropropyl)amino)cyclohexyl) amino)isoindolin-2-yl)piperidine- 2,6-dione |
| 637 | 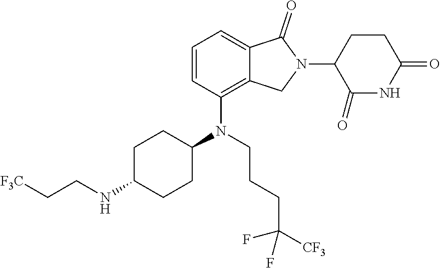 [SMILES: O=C1CCC(N2Cc3c(cccc3N(CCCC(F)(F)C(F)(F)F)[C@H]3CC[C@H](NCCC(F)(F)F)CC3)C2=O)C(=O)N1] | 3-(1-oxo-4-((4,4,5,5,5- pentafluoropentyl)((1r,4r)-4-((3,3,3- trifluoropropyl)amino)cyclohexyl) amino)isoindolin-2-yl)piperidine- 2,6-dione |
| 637A | 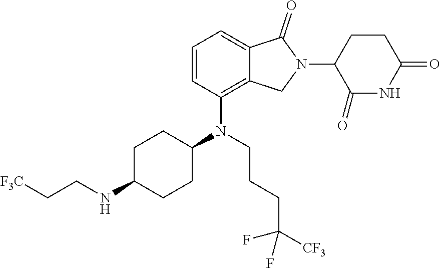 [SMILES: O=C1CCC(N2Cc3c(cccc3N(CCCC(F)(F)C(F)(F)F)[C@H]3CC[C@@H](NCCC(F)(F)F)CC3)C2=O)C(=O)N1] | 3-(1-oxo-4-((4,4,5,5,5- pentafluoropentyl)((1s,4s)-4- ((3,3,3- trifluoropropyl)amino)cyclohexyl) amino)isoindolin-2-yl)piperidine- 2,6-dione |
| 639 | 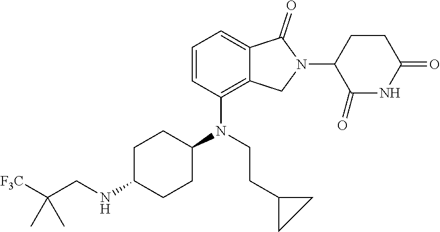 [SMILES: CC(C)(CN[C@H]1CC[C@H](N(CCC2CC2)c2cccc3c2CN(C2CCC(=O)NC2=O)C3=O)CC1)C(F)(F)F] | 3-(4-((2-cyclopropylethyl)((1r,4r)- 4-((3,3,3-trifluoro-2,2- dimethylpropyl)amino)cyclohexyl) amino)-1-oxoisoindolin-2- yl)piperidine-2,6-dione |
| 639A | 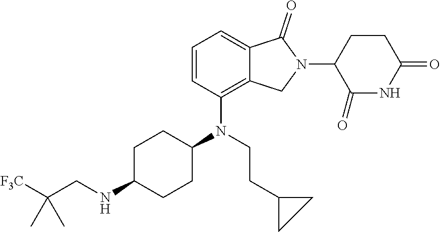 [SMILES: CC(C)(CN[C@H]1CC[C@@H](N(CCC2CC2)c2cccc3c2CN(C2CCC(=O)NC2=O)C3=O)CC1)C(F)(F)F] | 3-(4-((2-cyclopropylethyl)((1s,4s)- 4-((3,3,3-trifluoro-2,2- dimethylpropyl)amino)cyclohexyl) amino)-1-oxoisoindolin-2- yl)piperidine-2,6-dione |
| 642 | 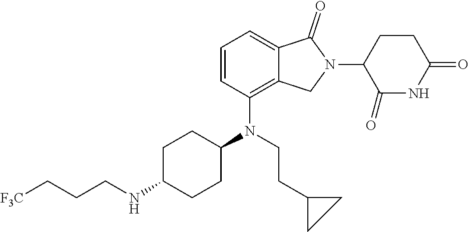 [SMILES: O=C1CCC(N2Cc3c(cccc3N(CCC3CC3)[C@H]3CC[C@H](NCCCC(F)(F)F)CC3)C2=O)C(=O)N1] | 3-(4-((2-cyclopropylethyl)((1r,4r)- 4-((4,4,4- trifluorobutyl)amino)cyclohexyl) amino)-1-oxoisoindolin-2- yl)piperidine-2,6-dione |
| 642A | 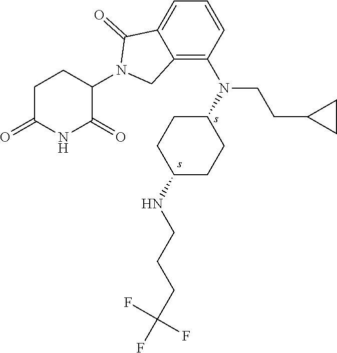 [SMILES: O=C1CCC(N2Cc3c(cccc3N(CCC3CC3)[C@H]3CC[C@@H](NCCCC(F)(F)F)CC3)C2=O)C(=O)N1] | 3-(4-((2-cyclopropylethyl)((1s,4s)- 4-((4,4,4- trifluorobutyl)amino)cyclohexyl) amino)-1-oxoisoindolin-2- yl)piperidine-2,6-dione |
| 643 | 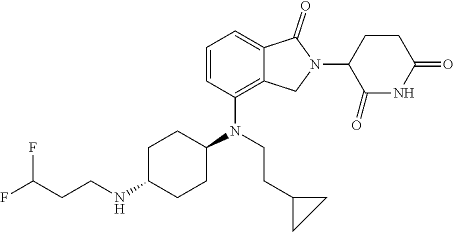 [SMILES: O=C1CCC(N2Cc3c(cccc3N(CCC3CC3)[C@H]3CC[C@H](NCCC(F)F)CC3)C2=O)C(=O)N1] | 3-(4-((2-cyclopropylethyl)((1r,4r)- 4-((3,3- difluoropropyl)amino)cyclohexyl) amino)-1-oxoisoindolin-2- yl)piperidine-2,6-dione |
| 643A | 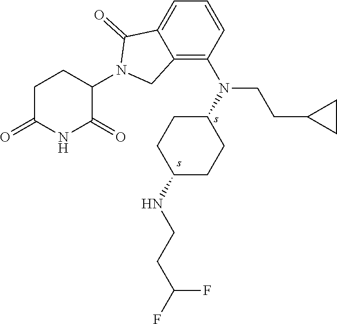 [SMILES: O=C1CCC(N2Cc3c(cccc3N(CCC3CC3)[C@H]3CC[C@@H](NCCC(F)F)CC3)C2=O)C(=O)N1] | 3-(4-((2-cyclopropylethyl)((1s,4s)- 4-((3,3- difluoropropyl)amino)cyclohexyl) amino)-1-oxoisoindolin-2- yl)piperidine-2,6-dione |
| 644 | 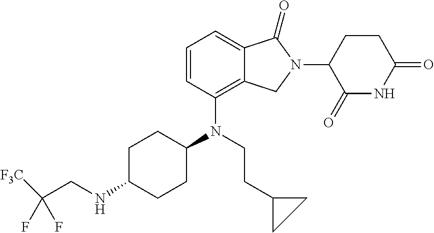 [SMILES: O=C1CCC(N2Cc3c(cccc3N(CCC3CC3)[C@H]3CC[C@H](NCC(F)(F)C(F)(F)F)CC3)C2=O)C(=O)N1] | 3-(4-((2-cyclopropylethyl)((1r,4r)- 4-((2,2,3,3,3-penta- fluoropropyl)amino)cyclohexyl) amino)-1-oxoisoindolin-2- yl)piperidine-2,6-dione |
| 644A | 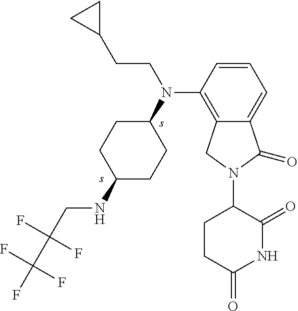 | 3-(4-((2-cyclopropylethyl)((1s,4s)- 4-((2,2,3,3,3-penta- fluoropropyl)amino)cyclohexyl) amino)-1-oxoisoindolin-2- yl)piperidine-2,6-dione |
| 645 | 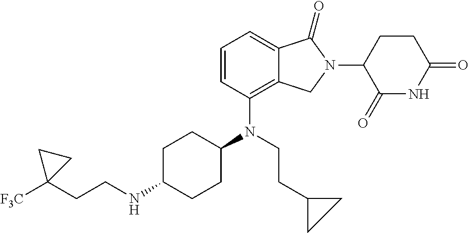 [SMILES: O=C1CCC(N2Cc3c(cccc3N(CCC3CC3)[C@H]3CC[C@H](NCCC4(C(F)(F)F)CC4)CC3)C2=O)C(=O)N1] | 3-(4-((2-cyclopropylethyl)((1r,4r)- 4-((2-(1- (trifluoromethyl)cyclopropyl)ethyl) amino)cyclohexyl)amino)-1- oxoisoindolin-2-yl)piperidine-2,6- dione |
| 645A | 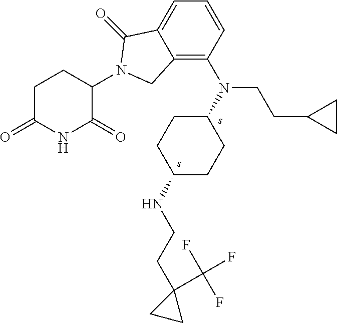 | 3-(4-((2-cyclopropylethyl)((1s,4s)- 4-((2-(1- (trifluoromethyl)cyclopropyl)ethyl) amino)cyclohexyl)amino)-1- oxoisoindolin-2-yl)piperidine-2,6- dione |
| 646 | 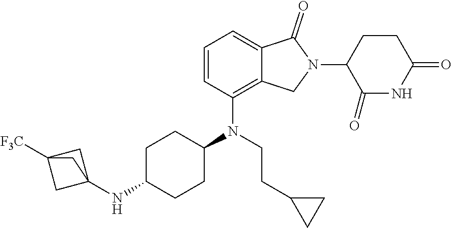 [SMILES: O=C1CCC(N2Cc3c(cccc3N(CCC3CC3)[C@H]3CC[C@H](NC45CC(C(F)(F)F)(C4)C5)CC3)C2=O)C(=O)N1] | 3-(4-((2-cyclopropylethyl)((1r,4r)- 4-((3-(trifluoro- methyl)bicyclo[1.1.1]pentan- 1-yl)amino)cyclohexyl)amino)- 1-oxoisoindolin-2-yl)piperidine- 2,6-dione |
| 646A | 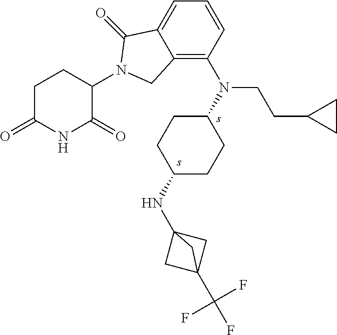 | 3-(4-((2-cyclopropylethyl)((1s,4s)- 4-((3-(trifluoro- methyl)bicyclo[1.1.1]pentan- 1-yl)amino)cyclohexyl)amino)- 1-oxoisoindolin-2-yl)piperidine- 2,6-dione |
| 647 | 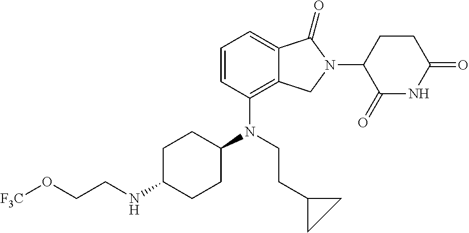 [SMILES: O=C1CCC(N2Cc3c(cccc3N(CCC3CC3)[C@H]3CC[C@H](NCCOC(F)(F)F)CC3)C2=O)C(=O)N1] | 3-(4-((2-cyclopropylethyl)((1r,4r)- 4-((2-(trifluoro- methoxy)ethyl)amino)cyclohexyl) amino)-1-oxoisoindolin-2- yl)piperidine-2,6-dione |
| 647A | 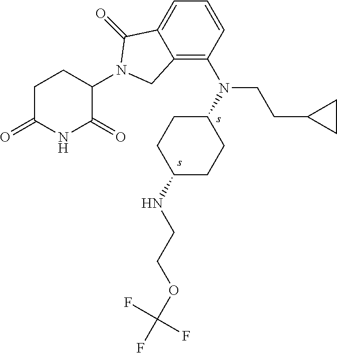 | 3-(4-((2-cycloproplylethyl)((1s,4s)- 4-((2- (trifluoromethoxy)ethyl)amino) cyclohexyl)amino)-1-oxoisoindolin-2- yl)piperidine-2,6-dione |
| 648 | 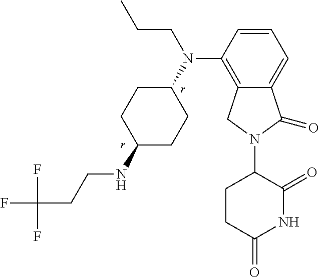 | 3-(1-oxo-4-(propyl((1r,4r)-4- ((3,3,3- trifluoropropyl)amino)cyclohexyl) amino)isoindolin-2-yl)piperidine- 2,6-dione |
| 648A | 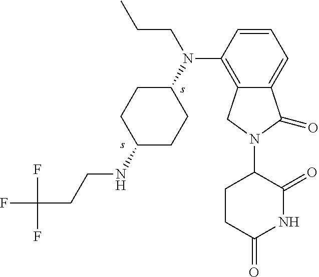 | 3-(1-oxo-4-(propyl((1s,4s)-4- ((3,3,3- trifluoropropyl)amino)cyclohexyl) amino)isoindolin-2-yl)piperidine- 2,6-dione |
| 649 | 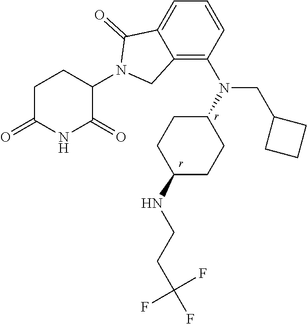 [SMILES: O=C1CCC(N2Cc3c(cccc3N(CC3CCC3)[C@H]3CC[C@H](NCCC(F)(F)F)CC3)C2=O)C(=O)N1] | 3-(4-((cyclobutylmethyl)((1r,4r)-4- ((3,3,3- trifluoropropyl)amino)cyclohexyl) amino)-1-oxoisoindolin-2- yl)piperidine-2,6-dione |
| 649A | 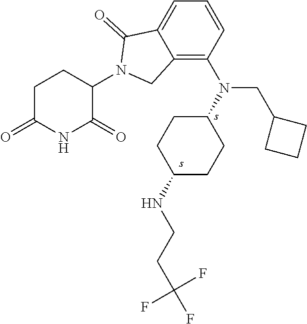 | 3-(4-((cyclobutylmethyl)((1s,4s)-4- ((3,3,3- trifluoropropyl)amino)cyclohexyl) amino)-1-oxoisoindolin-2- yl)piperidine-2,6-dione |
| 650 | 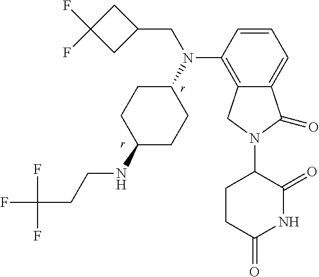 | 3-(4-(((3,3- difluorocyclobutyl)methyl)((1r,4r)- 4-((3,3,3- trifluoropropyl)amino)cyclohexyl) amino)-1-oxoisoindolin-2- yl)piperidine-2,6-dione |
| 650A | 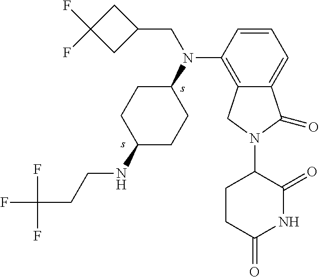 | 3-(4-(((3,3- difluorocyclobutyl)methyl)((1s,4s)- 4-((3,3,3- trifluoropropyl)amino)cyclohexyl) amino)-1-oxoisoindolin-2- yl)piperidine-2,6-dione |
| 651 | 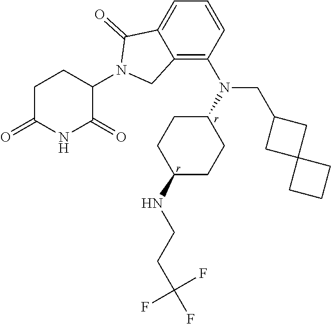 [SMILES: O=C1CCC(N2Cc3c(cccc3N(CC3CC4(CCC4)C3)[C@H]3CC[C@H](NCCC(F)(F)F)CC3)C2=O)C(=O)N1] | 3-(1-oxo-4-((spiro[3.3]heptan-2- ylmethyl)((1r,4r)-4-((3,3,3- trifluoropropyl)amino)cyclohexyl) amino)isoindolin-2-yl)piperidine- 2,6-dione |
| 651A | 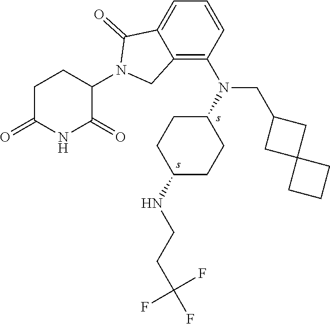 [SMILES: O=C1CCC(N2Cc3c(cccc3N(CC3CC4(CCC4)C3)[C@H]3CC[C@@H](NCCC(F)(F)F)CC3)C2=O)C(=O)N1] | 3-(1-oxo-4-((spiro[3.3]heptan-2- ylmethyl)((1s,4s)-4-((3,3,3- trifluoropropyl)amino)cyclohexyl) amino)isoindolin-2-yl)piperidine- 2,6-dione |
| 652 | 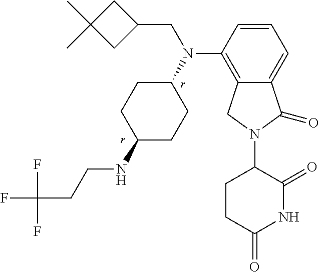 | 3-(4-(((3,3- dimethylcyclobutyl)methyl)((1r,4r)- 4-((3,3,3- trifluoropropyl)amino)cyclohexyl) amino)-1-oxoisoindolin-2- yl)piperidine-2,6-dione |
| 652A | 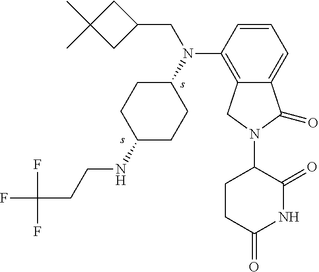 | 3-(4-(((3,3- dimethylcyclobutyl)methyl)((1s,4s)- 4-((3,3,3- trifluoropropyl)amino)cyclohexyl) amino)-1-oxoisoindolin-2- yl)piperidine-2,6-dione |
| 653 | 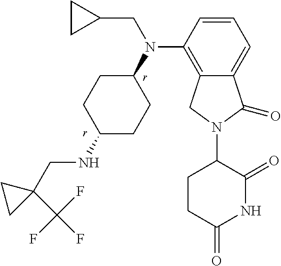 | 3-(4-((cyclopropylmethyl)((1r,4r)- 4-(((1- (trifluoromethyl)cyclopropyl)methyl) amino)cyclohexyl)amino)-1- oxoisoindolin-2-yl)piperidine-2,6- dione |
| 653A | 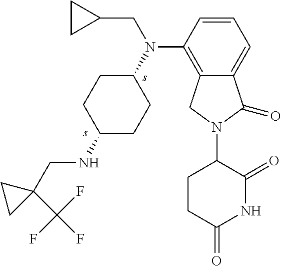 | 3-(4-((cyclopropylmethyl)((1s,4s)- 4-(((1- (trifluoromethyl)cyclopropyl)methyl) amino)cyclohexyl)amino)-1- oxoisoindolin-2-yl)piperidine-2,6- dione |
| 654 | 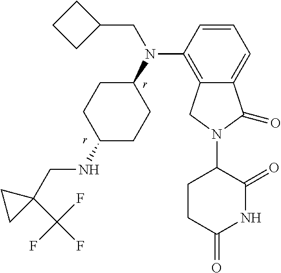 | 3-(4-((cyclobutylmethyl)((1r,4r)-4- (((1- (trifluoromethyl)cyclopropyl)methyl) amino)cyclohexyl)amino)-1- oxoisoindolin-2-yl)piperidine-2,6- dione |
| 654A | 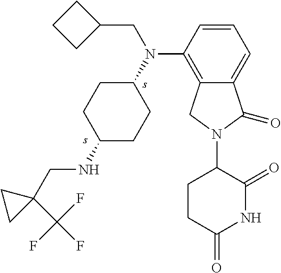 | 3-(4-((cyclobutylmethyl)((1s,4s)-4- (((1- (trifluoromethyl)cyclopropyl)methyl) amino)cyclohexyl)amino)-1- oxoisoindolin-2-yl)piperidine-2,6- dione |
| 655 | 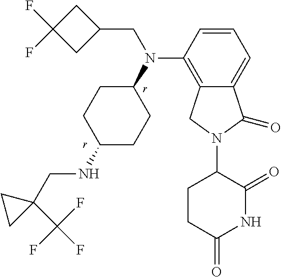 | 3-(4-(((3,3- difluorocyclobutyl)methyl)((1r,4r)- 4-(((1- (trifluoromethyl)cyclopropyl)methyl) amino)cyclohexyl)amino)-1- oxoisoindolin-2-yl)piperidine-2,6- dione |
| 655A | 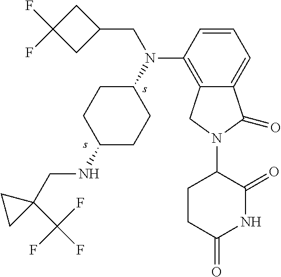 | 3-(4-(((3,3- difluorocyclobutyl)methyl)((1s,4s)- 4-(((1- (trifluoromethyl)cyclopropyl)methyl) amino)cyclohexyl)amino)-1- oxoisoindolin-2-yl)piperidine-2,6- dione |
| 656 | 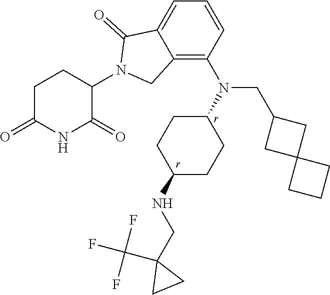 [SMILES: O=C1CCC(N2Cc3c(cccc3N(CC3CC4(CCC4)C3)[C@H]3CC[C@H](NCC4(C(F)(F)F)CC4)CC3)C2=O)C(=O)N1] | 3-(1-oxo-4-((spiro[3.3]heptan-2- ylmethyl)((1r,4r)-4-(((1- (trifluoromethyl)cyclopropyl)methyl) amino)cyclohexyl)amino)isoindolin- 2-yl)piperidine-2,6-dione |
| 656A | 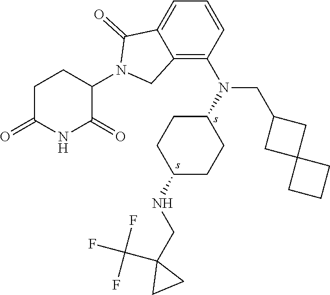 [SMILES: O=C1CCC(N2Cc3c(cccc3N(CC3CC4(CCC4)C3)[C@H]3CC[C@@H](NCC4(C(F)(F)F)CC4)CC3)C2=O)C(=O)N1] | 3-(1-oxo-4-((spiro[3.3]heptan-2- ylmethyl)((1s,4s)-4-(((1- (trifluoromethyl)cyclopropyl)methyl) amino)cyclohexyl)amino)isoindolin- 2-yl)piperidine-2,6-dione |
| 657 | 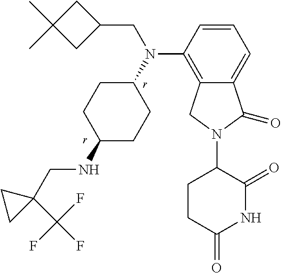 | 3-(4-(((3,3- dimethylcyclobutyl)methyl)((1r,4r)- 4-(((1- (trifluoromethyl)cyclopropyl)methyl) amino)cyclohexyl)amino)-1- oxoisoindolin-2-yl)piperidine-2,6- dione |
| 657A | 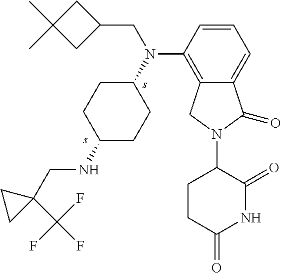 | 3-(4-(((3,3- dimethylcyclobutyl)methyl)((1s,4s)- 4-(((1- (trifluoromethyl)cyclopropyl)methyl) amino)cyclohexyl)amino)-1- oxoisoindolin-2-yl)piperidine-2,6- dione |
| 658 | 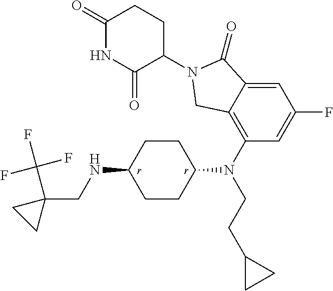 [SMILES: O=C1CCC(N2Cc3c(cc(F)cc3N(CCC3CC3)[C@H]3CC[C@H](NCC4(C(F)(F)F)CC4)CC3)C2=O)C(=O)N1] | 3-(4-((2-cyclopropylethyl)((1r,4r)- 4-(((1- (trifluoromethyl)cyclopropyl)methyl) amino)cyclohexyl)amino)-6- fluoro-1-oxoisoindolin-2- yl)piperidine-2,6-dione |
| 658A | 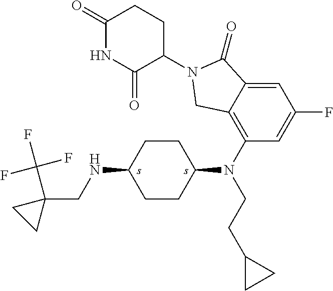 [SMILES: O=C1CCC(N2Cc3c(cc(F)cc3N(CCC3CC3)[C@H]3CC[C@@H](NCC4(C(F)(F)F)CC4)CC3)C2=O)C(=O)N1] | 3-(4-((2-cyclopropylethyl)((1s,4s)- 4-(((1- (trifluoromethyl)cyclopropyl)methyl) amino)cyclohexyl)amino)-6- fluoro-1-oxoisoindolin-2- yl)piperidine-2,6-dione |
| 659 | 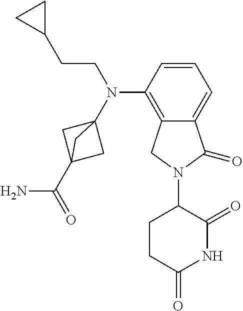 [SMILES: NC(=O)C12CC(N(CCC3CC3)c3cccc4c3CN(C3CCC(=O)NC3=O)C4=O)(C1)C2] | 3-((2-cyclopropylethyl)(2-(2,6- dioxopiperidin-3-yl)-1- oxoisoindolin-4- yl)amino)bicyclo[1.1.1]pentane-1- carboxamide |
| 660 | 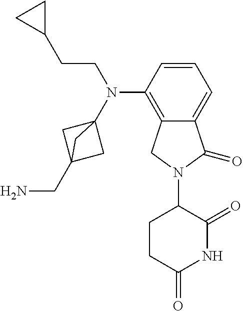 [SMILES: NCC12CC(N(CCC3CC3)c3cccc4c3CN(C3CCC(=O)NC3=O)C4=O)(C1)C2] | 3-(4-((3- (aminomethyl)bicyclo[1.1.1]pentan- 1-yl)(2-cyclopropylethyl)amino)- 1-oxoisoindolin-2-yl)piperidine- 2,6-dione |
| 662 | 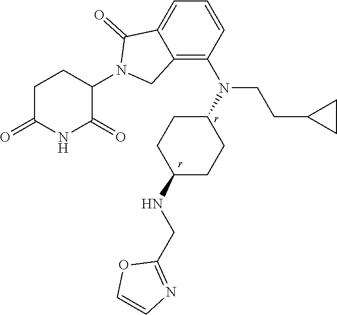 [SMILES: O=C1CCC(N2Cc3c(cccc3N(CCC3CC3)[C@H]3CC[C@H](NCc4ncco4)CC3)C2=O)C(=O)N1] | 3-(4-((2-cyclopropylethyl)((1r,4r)- 4-((oxazol-2- ylmethyl)amino)cyclohexyl)amino)- 1-oxoisoindolin-2-yl)piperidine- 2,6-dione |
| 663 | 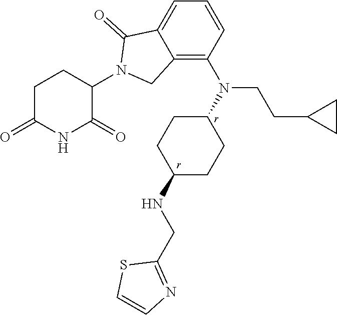 [SMILES: O=C1CCC(N2Cc3c(cccc3N(CCC3CC3)[C@H]3CC[C@H](NCc4nccs4)CC3)C2=O)C(=O)N1] | 3-(4-((2-cyclopropylethyl)((1r,4r)- 4-((thiazol-2- ylmethyl)amino)cyclohexyl)amino)- 1-oxoisoindolin-2-yl)piperidine- 2,6-dione |
| 664 | 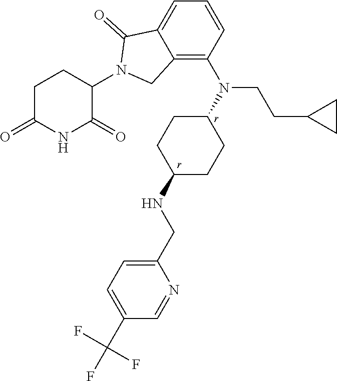 [SMILES: O=C1CCC(N2Cc3c(cccc3N(CCC3CC3)[C@H]3CC[C@H](NCc4ccc(C(F)(F)F)cn4)CC3)C2=O)C(=O)N1] | 3-(4-((2-cyclopropylethyl)((1r,4r)- 4-(((5-(trifluoromethyl)pyridin-2- yl)methyl)amino)cyclohexyl)amino)- 1-oxoisoindolin-2-yl)piperidine- 2,6-dione |
| 665 | 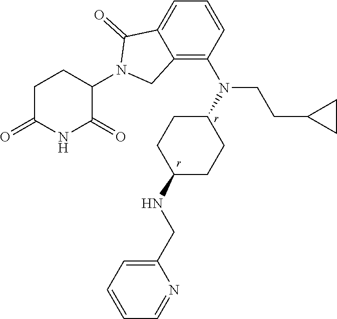 [SMILES: O=C1CCC(N2Cc3c(cccc3N(CCC3CC3)[C@H]3CC[C@H](NCc4ccccn4)CC3)C2=O)C(=O)N1] | 3-(4-((2-cyclopropylethyl)((1r,4r)- 4-((pyridin-2- ylmethyl)amino)cyclohexyl)amino)- 1-oxoisoindolin-2-yl)piperidine- 2,6-dione |
| 666 | 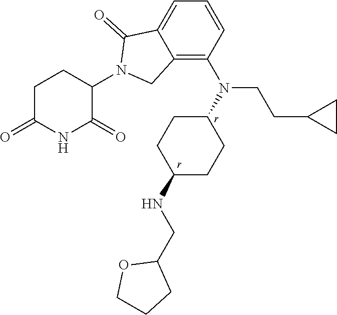 [SMILES: O=C1CCC(N2Cc3c(cccc3N(CCC3CC3)[C@H]3CC[C@H](NCC4CCCO4)CC3)C2=O)C(=O)N1] | 3-(4-((2-cyclopropylethyl)((1r,4r)- 4-(((tetrahydrofuran-2- yl)methyl)amino)cyclohexyl)amino)- 1-oxoisoindolin-2-yl)piperidine- 2,6-dione |
| 668 | 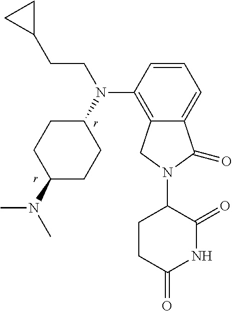 | 3-(4-((2-cyclopropylethyl)((1r,4r)- 4- (dimethylamino)cyclohexyl)amino)- 1-oxoisoindolin-2-yl)piperidine- 2,6-dione |
| 669 | 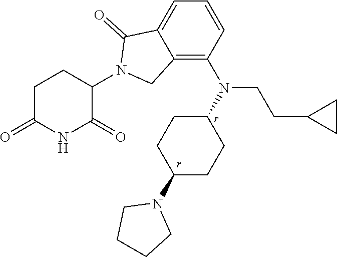 [SMILES: O=C1CCC(N2Cc3c(cccc3N(CCC3CC3)[C@H]3CC[C@H](N4CCCC4)CC3)C2=O)C(=O)N1] | 3-(4-((2-cyclopropylethyl)((1r,4r)- 4-(pyrrolidin-1- yl)cyclohexyl)amino)-1- oxoisoindolin-2-yl)piperidine-2,6- dione |
| 669A | 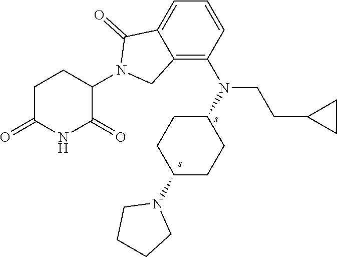 [SMILES: O=C1CCC(N2Cc3c(cccc3N(CCC3CC3)[C@H]3CC[C@@H](N4CCCC4)CC3)C2=O)C(=O)N1] | 3-(4-((2-cyclopropylethyl)((1s,4s)- 4-(pyrrolidin-1- yl)cyclohexyl)amino)-1- oxoisoindolin-2-yl)piperidine-2,6- dione |
| 670 | 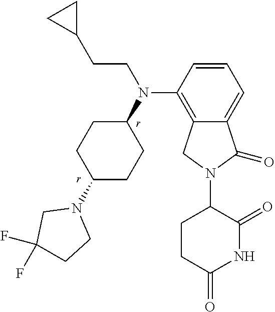 | 3-(4-((2-cyclopropylethyl)((1r,4r)- 4-(3,3-difluoropyrrolidin-1- yl)cyclohexyl)amino)-1- oxoisoindolin-2-yl)piperidine-2,6- dione |
| 670A | 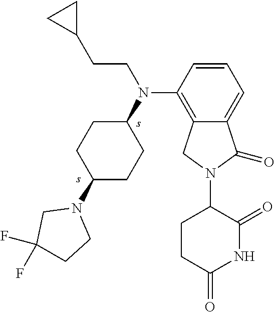 | 3-(4-((2-cyclopropylethyl)((1s,4s)- 4-(3,3-difluoropyrrolidin-1- yl)cyclohexyl)amino)-1- oxoisoindolin-2-yl)piperidine-2,6- dione |
| 671 | 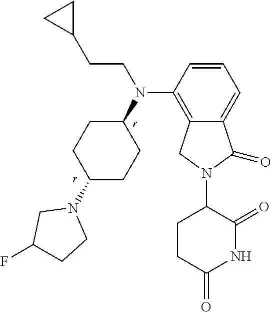 | 3-(4-((2-cyclopropylethyl)((1r,4r)- 4-(3-fluoropyrrolidin-1- yl)cyclohexyl)amino)-1- oxoisoindolin-2-yl)piperidine-2,6- dione |
| 671A | 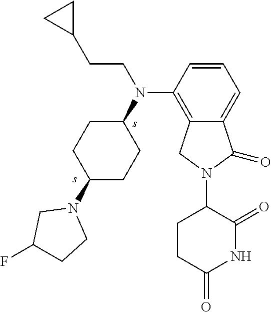 | 3-(4-((2-cyclopropylethyl)((1s,4s)- 4-(3-fluoropyrrolidin-1- yl)cyclohexyl)amino)-1- oxoisoindolin-2-yl)piperidine-2,6- dione |
| 672 | 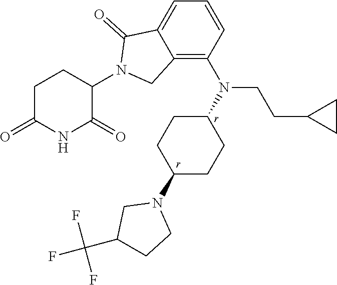 [SMILES: O=C1CCC(N2Cc3c(cccc3N(CCC3CC3)[C@H]3CC[C@H](N4CCC(C(F)(F)F)C4)CC3)C2=O)C(=O)N1] | 3-(4-((2-cyclopropylethyl)((1r,4r)- 4-(3-(trifluoromethyl)pyrrolidin-1- yl)cyclohexyl)amino)-1- oxoisoindolin-2-yl)piperidine-2,6- dione |
| 672A | 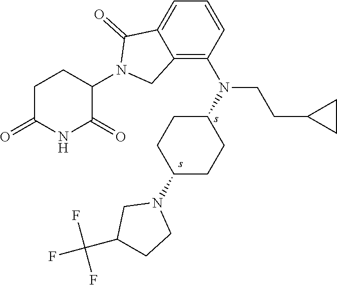 [SMILES: O=C1CCC(N2Cc3c(cccc3N(CCC3CC3)[C@H]3CC[C@@H](N4CCC(C(F)(F)F)C4)CC3)C2=O)C(=O)N1] | 3-(4-((2-cyclopropylethyl)((1s,4s)- 4-(3-(trifluoromethyl)pyrrolidin-1- yl)cyclohexyl)amino)-1- oxoisoindolin-2-yl)piperidine-2,6- dione |
| 673 | 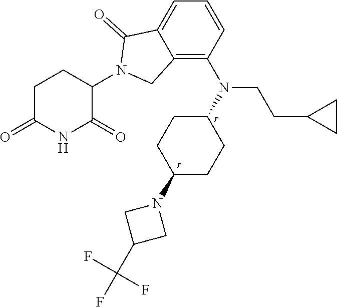 [SMILES: O=C1CCC(N2Cc3c(cccc3N(CCC3CC3)[C@H]3CC[C@H](N4CC(C(F)(F)F)C4)CC3)C2=O)C(=O)N1] | 3-(4-((2-cyclopropylethyl)((1r,4r)- 4-(3-(trifluoromethyl)azetidin-1- yl)cyclohexyl)amino)-1- oxoisoindolin-2-yl)piperidine-2,6- dione |
| 673A | 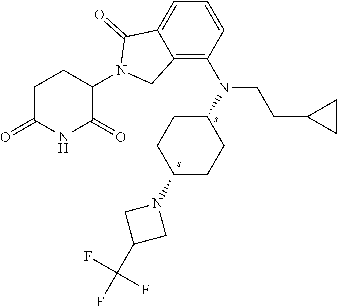 [SMILES: O=C1CCC(N2Cc3c(cccc3N(CCC3CC3)[C@H]3CC[C@@H](N4CC(C(F)(F)F)C4)CC3)C2=O)C(=O)N1] | 3-(4-((2-cyclopropylethyl)((1s,4s)- 4-(3-(trifluoromethyl)azetidin-1- yl)cyclohexyl)amino)-1- oxoisoindolin-2-yl)piperidine-2,6- dione |
| 674 | 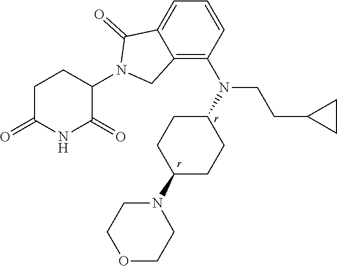 [SMILES: O=C1CCC(N2Cc3c(cccc3N(CCC3CC3)[C@H]3CC[C@H](N4CCOCC4)CC3)C2=O)C(=O)N1] | 3-(4-((2-cyclopropylethyl)((1r,4r)- 4-morpholinocyclohexyl)amino)-1- oxoisoindolin-2-yl)piperidine-2,6- dione |
| 674A | 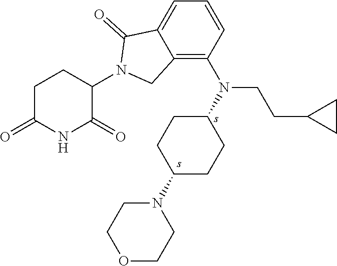 [SMILES: O=C1CCC(N2Cc3c(cccc3N(CCC3CC3)[C@H]3CC[C@@H](N4CCOCC4)CC3)C2=O)C(=O)N1] | 3-(4-((2-cyclopropylethyl)((1s,4s)- 4-morpholinocyclohexyl)amino)-1- oxoisoindolin-2-yl)piperidine-2,6- dione |
| 675 | 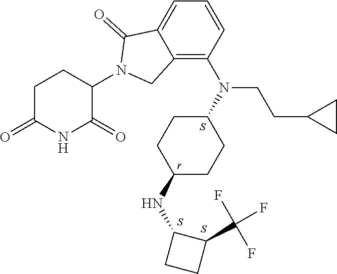 | 3-(4-((2-cyclopropylethyl)((1S,4r)- 4-(((1S,2S)-2- (trifluoromethyl)cyclobutyl)amino) cyclohexyl)amino)-1- oxoisoindolin-2-yl)piperidine-2,6- dione |
| 675A | 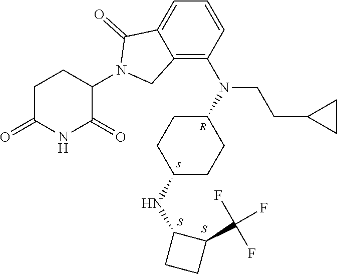 | 3-(4-((2-cyclopropylethyl)((1R,4s)- 4-(((1S,2S)-2- (trifluoromethyl)cyclobutyl)amino) cyclohexyl)amino)-1- oxoisoindolin-2-yl)piperidine-2,6- dione |
| 678 | 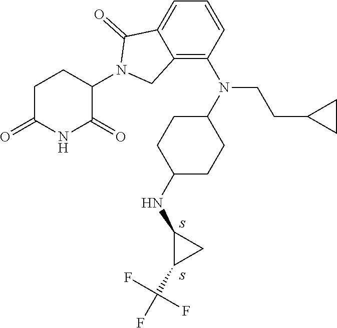 | 3-(4-((2-cyclopropylethyl)(4- (((1S,2S)-2- (trifluoromethyl)cyclopropyl)amino) cyclohexyl)amino)-1- oxoisoindolin-2-yl)piperidine-2,6- dione |
| 679 | 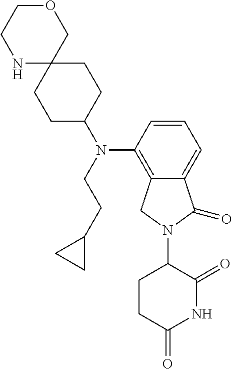 [SMILES: O=C1CCC(N2Cc3c(cccc3N(CCC3CC3)C3CCC4(CC3)COCCN4)C2=O)C(=O)N1] | 3-(4-((2-cyclopropylethyl)(4-oxa-1- azaspiro[5.5]undecan-9-yl)amino)- 1-oxoisoindolin-2-yl)piperidine- 2,6-dione |
| 680 | 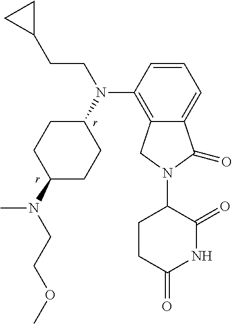 [SMILES: COCCN(C)[C@H]1CC[C@H](N(CCC2CC2)c2cccc3c2CN(C2CCC(=O)NC2=O)C3=O)CC1] | 3-(4-((2-cyclopropylethyl)((1r,4r)- 4-((2-methoxyethyl) (methyl)amino)cyclohexyl) amino)-1-oxoisoindolin-2- yl)piperidine-2,6-dione |
| 680A | 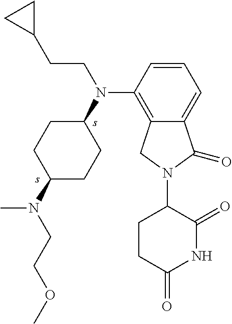 [SMILES: COCCN(C)[C@H]1CC[C@@H](N(CCC2CC2)c2cccc3c2CN(C2CCC(=O)NC2=O)C3=O)CC1] | 3-(4-((2-cyclopropylethyl)((1s,4s)- 4-((2-methoxyethyl) (methyl)amino)cyclohexyl) amino)-1-oxoisoindolin-2- yl)piperidine-2,6-dione |
| 681 | 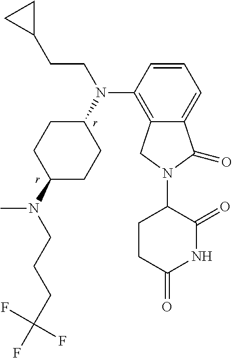 [SMILES: CN(CCCC(F)(F)F)[C@H]1CC[C@H](N(CCC2CC2)c2cccc3c2CN(C2CCC(=O)NC2=O)C3=O)CC1] | 3-(4-((2-cyclopropylethyl)((1r,4r)- 4-(methyl(4,4,4- trifluorobutyl)amino)cyclohexyl) amino)-1-oxoisoindolin-2- yl)piperidine-2,6-dione |
| 681A | 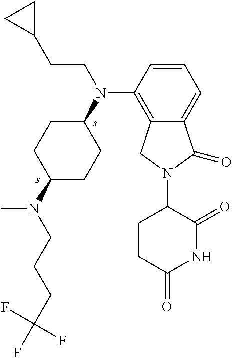 [SMILES: CN(CCCC(F)(F)F)[C@H]1CC[C@@H](N(CCC2CC2)c2cccc3c2CN(C2CCC(=O)NC2=O)C3=O)CC1] | 3-(4-((2-cyclopropylethyl)((1s,4s)- 4-(methyl(4,4,4- trifluorobutyl)amino)cyclohexyl) amino)-1-oxoisoindolin-2- yl)piperidine-2,6-dione |
| 682 | 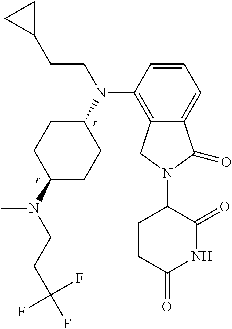 [SMILES: CN(CCC(F)(F)F)[C@H]1CC[C@H](N(CCC2CC2)c2cccc3c2CN(C2CCC(=O)NC2=O)C3=O)CC1] | 3-(4-((2-cyclopropylethyl)((1r,4r)- 4-(methyl(3,3,3- trifluoropropyl)amino)cyclohexyl) amino)-1-oxoisoindolin-2- yl)piperidine-2,6-dione |
| 682A | 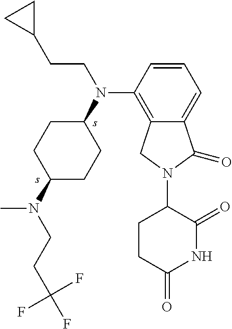 [SMILES: CN(CCC(F)(F)F)[C@H]1CC[C@@H](N(CCC2CC2)c2cccc3c2CN(C2CCC(=O)NC2=O)C3=O)CC1] | 3-(4-((2-cyclopropylethyl)((1s,4s)- 4-(methyl(3,3,3- trifluoropropyl)amino)cyclohexyl) amino)-1-oxoisoindolin-2- yl)piperidine-2,6-dione |
| 683 | 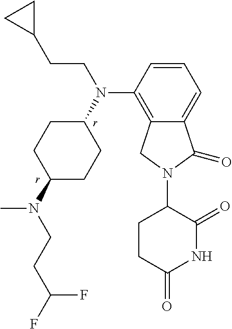 [SMILES: CN(CCC(F)F)[C@H]1CC[C@H](N(CCC2CC2)c2cccc3c2CN(C2CCC(=O)NC2=O)C3=O)CC1] | 3-(4-((2-cyclopropylethyl)((1r,4r)- 4-((3,3-difluoro- propyl)(methyl)amino)cyclohexyl) amino)-1-oxoisoindolin-2- yl)piperidine-2,6-dione |
| 683A | 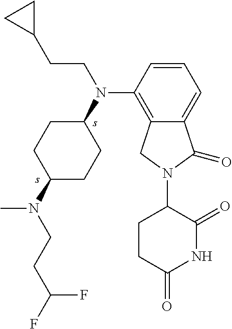 [SMILES: CN(CCC(F)F)[C@H]1CC[C@@H](N(CCC2CC2)c2cccc3c2CN(C2CCC(=O)NC2=O)C3=O)CC1] | 3-(4-((2-cyclopropylethyl)((1s,4s)- 4-((3,3-difluoropropyl) (methyl)amino)cyclohexyl) amino)-1-oxoisoindolin-2- yl)piperidine-2,6-dione |
| 703 | 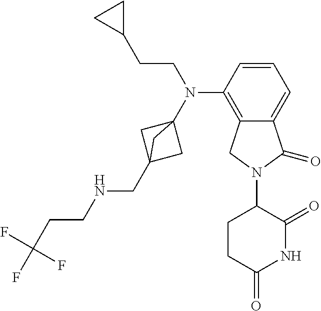 [SMILES: O=C1CCC(N2Cc3c(cccc3N(CCC3CC3)C34CC(CNCCC(F)(F)F)(C3)C4)C2=O)C(=O)N1] | 3-(4-((2-cyclopropylethyl)(3- (((3,3,3-trifluoropropyl) amino)methyl)bicyclo [1.1.1]pentan-1-yl)amino)-1- oxoisoindolin-2-yl)piperidine-2,6- dione |
| 708 | 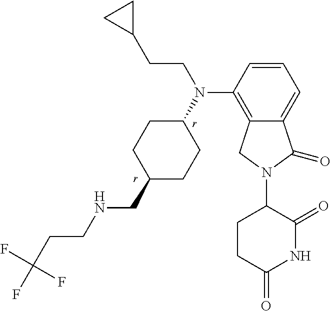 [SMILES: O=C1CCC(N2Cc3c(cccc3N(CCC3CC3)[C@H]3CC[C@H](CNCCC(F)(F)F)CC3)C2=O)C(=O)N1] | 3-(4-((2-cyclopropylethyl)((1r,4r)- 4-(((3,3,3-trifluoropropyl) amino)methyl)cyclohexyl) amino)-1-oxoisoindolin-2- yl)piperidine-2,6-dione |
| 708A | 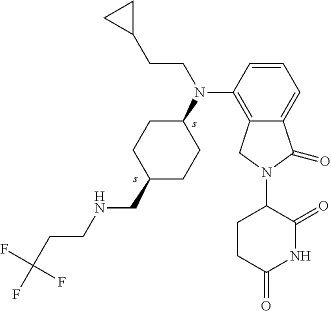 [SMILES: O=C1CCC(N2Cc3c(cccc3N(CCC3CC3)[C@H]3CC[C@@H](CNCCC(F)(F)F)CC3)C2=O)C(=O)N1] | 3-(4-((2-cyclopropylethyl)((1s,4s)- 4-(((3,3,3-trifluoropropyl) amino)methyl)cyclohexyl) amino)-1-oxoisoindolin-2- yl)piperidine-2,6-dione |
| 711 | 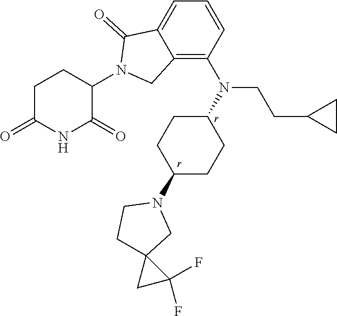 [SMILES: O=C1CCC(N2Cc3c(cccc3N(CCC3CC3)[C@H]3CC[C@H](N4CCC5(C4)CC5(F)F)CC3)C2=O)C(=O)N1] | 3-(4-((2-cyclopropylethyl)((1r,4r)- 4-(1,1-difluoro-5- azaspiro[2.4]heptan-5- yl)cyclohexyl)amino)-1- oxoisoindolin-2-yl)piperidine-2,6- dione |
| 711A | 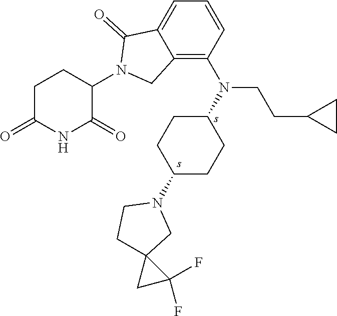 [SMILES: O=C1CCC(N2Cc3c(cccc3N(CCC3CC3)[C@H]3CC[C@@H](N4CCC5(C4)CC5(F)F)CC3)C2=O)C(=O)N1] | 3-(4-((2-cyclopropylethyl)((1s,4s)- 4-(1,1-difluoro-5- azaspiro[2.4]heptan-5- yl)cyclohexyl)amino)-1- oxoisoindolin-2-yl)piperidine-2,6- dione |
| 712 | 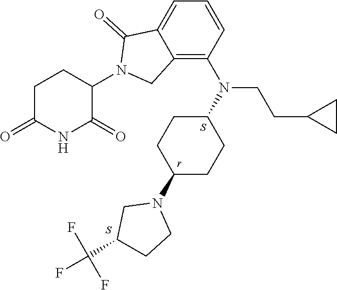 | 3-(4-((2-cyclopropylethyl)((1S,4r)- 4-((S)-3- (trifluoromethyl)pyrrolidin-1- yl)cyclohexyl)amino)-1- oxoisoindolin-2-yl)piperidine-2,6- dione |
| 712A | 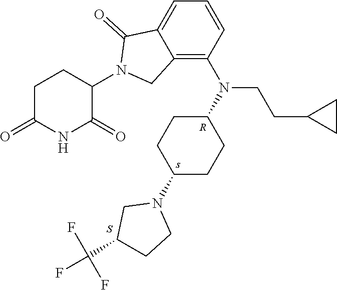 | 3-(4-((2-cyclopropylethyl)((1R,4s)- 4-((S)-3- (trifluoromethyl)pyrrolidin-1- yl)cyclohexyl)amino)-1- oxoisoindolin-2-yl)piperidine-2,6- dione |
| 713 | 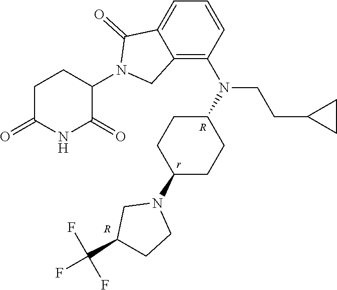 | 3-(4-((2-cyclopropylethyl)((1R,4r)- 4-((R)-3- (trifluoromethyl)pyrrolidin-1- yl)cyclohexyl)amino)-1- oxoisoindolin-2-yl)piperidine-2,6- dione |
| 713A | 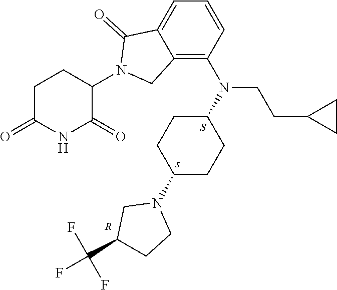 | 3-(4-((2-cyclopropylethyl)((1S,4s)- 4-((R)-3- (trifluoromethyl)pyrrolidin-1- yl)cyclohexyl)amino)-1- oxoisoindolin-2-yl)piperidine-2,6- dione |
| 714 | 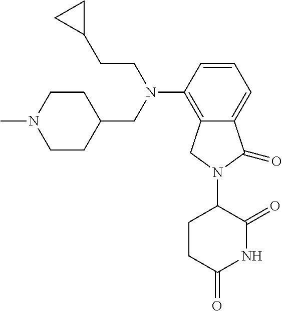 [SMILES: CN1CCC(CN(CCC2CC2)c2cccc3c2CN(C2CCC(=O)NC2=O)C3=O)CC1] | 3-(4-((2-cyclopropylethyl)((1- methylpiperidin-4- yl)methyl)amino)-1-oxoisoindolin- 2-yl)piperidine-2,6-dione |
| 715 | 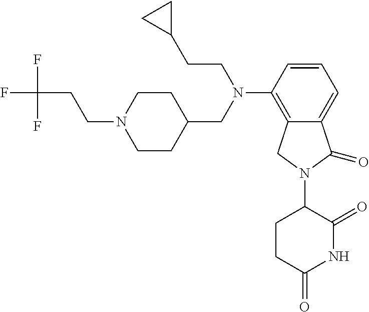 [SMILES: O=C1CCC(N2Cc3c(cccc3N(CCC3CC3)CC3CCN(CCC(F)(F)F)CC3)C2=O)C(=O)N1] | 3-(4-((2-cyclopropylethyl)((1- (3,3,3-trifluoropropyl)piperidin-4- yl)methyl)amino)-1-oxoisoindolin- 2-yl)piperidine-2,6-dione |
| 716 | 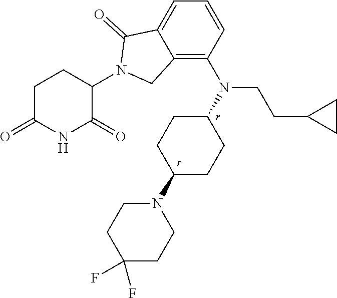 | 3-(4-((2-cyclopropylethyl)((1r,4r)- 4-(4,4-difluoropiperidin-1- yl)cyclohexyl)amino)-1- oxoisoindolin-2-yl)piperidine-2,6- dione |
| 716A | 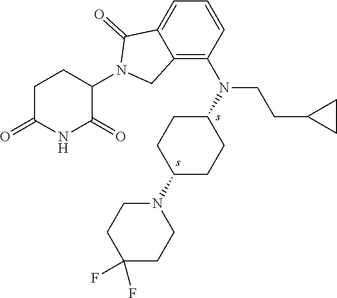 | 3-(4-((2-cyclopropylethyl)((1s,4s)- 4-(4,4-difluoropiperidin-1- yl)cyclohexyl)amino)-1- oxoisoindolin-2-yl)piperidine-2,6- dione |

or a pharmaceutically acceptable salt, enantiomer, stereoisomer or prodrug thereof.

40. A pharmaceutical composition comprising:
the compound of claim 39, or
the pharmaceutically acceptable salt, enantiomer, stereoisomer or prodrug thereof, and
optionally a pharmaceutically acceptable excipient or carrier.

41. (canceled)

42. (canceled)

43. (canceled)

44. (canceled)

45. (canceled)

46. (canceled)

47. (canceled)

48. A method of degrading or reducing casein kinase 1 alpha (CK1α) in a subject in need thereof, the method comprising administering to the subject an effective amount of a composition of comprising the compound according to claim 39.

49. A method of inhibiting casein kinase 1 alpha (CK1α) activity in a subject in need thereof, the method comprising administering to the subject an effective amount of a composition of comprising the compound according to claim 39.

50. A method of preventing or treating blood cancer in a subject in need thereof, the method comprising administering to the subject an effective amount of a composition of comprising the compound according to claim 39.

51. A pharmaceutical composition comprising:
the compound of claim 19, or the pharmaceutically acceptable salt, enantiomer, stereoisomer or prodrug thereof, and
optionally a pharmaceutically acceptable excipient or carrier.

52. A method of degrading or reducing casein kinase 1 alpha (CK1α) in a subject in need thereof, the method comprising administering to the subject an effective amount of the pharmaceutical composition of claim 51.

53. A method of inhibiting casein kinase 1 alpha (CK1α) activity in a subject in need thereof, the method comprising administering to the subject an effective amount of the pharmaceutical composition of claim 51.

54. A method of preventing or treating blood cancer in a subject in need thereof, the method comprising administering to the subject an effective amount of the pharmaceutical composition of claim 51.

55. The method of claim 54, wherein the blood cancer is leukemia, lymphoma, or myeloma.

56. The method of claim 55, wherein the leukemia is selected from the group consisting of acute myeloid leukemia (AML), acute lymphoblastic leukemia or acute lymphocytic leukemia (ALL), T-cell acute lymphoblastic leukemia (T-ALL), B-cell acute lymphoblastic leukemia (B-ALL), chronic myeloid leukemia (CML), chronic lymphocytic leukemia (CLL), hairy cell leukemia (HCL), T-cell prolymphocytic leukemia (T-PLL), Large granular lymphocytic leukemia, adult T-cell leukemia, chronic eosinophilic leukemia (CEL) and myelodysplastic syndrome (MDS).

57. The method of claim 55, wherein the lymphoma is selected from the group consisting of Hodgkin's lymphoma, diffuse large B-cell lymphoma (DLBCL), follicular lymphoma, small lymphocytic lymphoma (SLL), mantle cell lymphoma (MCL), marginal zone lymphomas, Burkitt lymphoma, lymphoplasmacytic lymphoma, primary central nervous system (CNS) lymphoma and peripheral T-cell lymphoma.

58. The method of claim 55, the myeloma is selected from the group consisting of multiple myeloma, light chain myeloma, non-secretory myeloma, solitary plasmacytoma, extramedullary plasmacytoma, monoclonal gammopathy of undetermined significance (MGUS), smoldering multiple myeloma (SMM), immunoglobulin D (IgD) myeloma and immunoglobulin E (IgE) myeloma.
